(19) United States
(12) Patent Application Publication
(10) Pub. No.: US 2023/0159453 A1
(43) Pub. Date: May 25, 2023

(54) INHIBITORS OF NOROVIRUS AND CORONAVIRUS REPLICATION

(71) Applicant: COCRYSTAL PHARMA, INC., Bothell, WA (US)

(72) Inventor: Irina C. Jacobson, Sammamish, WA (US)

(21) Appl. No.: 17/912,749

(22) PCT Filed: Mar. 17, 2021
(86) PCT No.: PCT/US21/22656

§ 371 Date: Sep. 19, 2022

Related U.S. Application Data
(60) Provisional application No. 63/027,495, filed on May 20, 2020.
(60) Provisional application No. 63/008,183, filed on Apr. 10, 2020.

(51) Int. Cl. C07D 207/38; C07D 471/10; C07D 401/12; C07F 9/40; C07D 211/76; C07C 237/22; C07D 267/14; C07D 241/04; C07D 413/12; C07D 403/12; C07D 403/06; C07D 491/107; C07D 209/54

(52) U.S. Cl. CPC C07D 207/38; C07D 471/10; C07D 401/12; C07F 9/4006; C07D 211/76; C07C 237/22; C07D 267/14; C07D 241/04; C07D 413/12; C07D 403/12; C07D 403/06; C07D 491/107; C07D 209/54

(57) ABSTRACT

Compounds of Formula (I) and methods of inhibiting the replication of viruses in a biological sample or patient, of reducing the amount of viruses in a biological sample or patient, and of treating a vims infection in a patient, comprising administering to said biological sample or patient an effective amount of a compound represented by Formula (I), a compound of Table A or B or a pharmaceutically acceptable salt thereof.

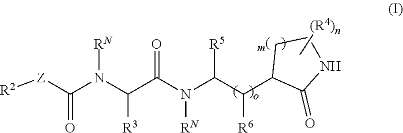

### FIELD OF THE DISCLOSURE

This disclosure relates generally to inhibitors of norovirus and coronavirus replication, and methods of treating or preventing norovirus and coronavirus infections by administering the inhibitors to a patient in need of treatment thereof.

### BACKGROUND

Noroviruses are important enteric pathogens involved in non-bacterial gastroenteritis outbreaks worldwide. Noroviruses mainly occur from person to person via the fecal-oral route but also through contaminated food or water. Indirect contamination is also possible owing to the persistence of the virus in the environment. Human noroviruses belong to the genus Norovirus, family Caliciviridae and are non-enveloped viruses with a positive-sense, single-stranded RNA genome. Norovirus strains are classified into seven groups. Viruses belonging to groups GI, GII, and GIV infect humans, while groups GII, GIII, GIV, GV, GVI and GVII NoVs have been described in animals.

Coronaviruses are a family of common viruses that cause a range of illnesses in humans from the common cold to severe acute respiratory syndrome (SARS). Coronaviruses can also cause a number of diseases in animals. Coronaviruses are enveloped, positive-stranded RNA viruses whose name derives from their characteristic crown-like appearance in electron micrographs. Coronaviruses are classified as a family within the Nidovirales order, viruses that replicate using a nested set of mRNAs. The coronavirus subfamily is further classified into four genera: alpha, beta, gamma, and delta coronaviruses. The human coronaviruses (HCoVs) are in two of these genera: alpha coronaviruses (including HCoV-229E and HCoV-NL63) and beta coronaviruses (including HCoV-HKU1, HCoV-OC43, Middle East respiratory syndrome coronavirus (MERS-CoV), the severe acute respiratory syndrome coronavirus (SARS-CoV), and SARS-CoV-2).

In 2012, a novel coronavirus emerged in Saudi Arabia and became known as Middle East Respiratory Syndrome coronavirus (MERS-CoV). About half of reported cases of MERS-CoV infection have resulted in death and a majority of reported cases have occurred in older to middle age men. Only a small number of reported cases involved subjects with mild respiratory illness. Human to human transmission of MERS-CoV has been found to be possible, but very limited. Another novel coronavirus emerged in Wuhan, China in late 2019. This virus is known as SARS-CoV-2, 2019-nCoV, or Wuhan coronavirus, and it the cause of a worldwide pandemic in late 2019 and 2020.

Given the widespread transmission and potential health effects of these viruses, there is a need for drugs for treating norovirus and coronavirus infections.

### SUMMARY

The present disclosure generally relates to methods of treating norovirus and coronavirus infections, to methods of inhibiting the replication of noroviruses and coronaviruses, to methods of reducing the amount of noroviruses and coronaviruses, and to compounds and compositions that can be employed for such methods.

The disclosure provides compounds of Formula (I), and pharmaceutically acceptable salts thereof:

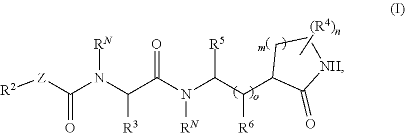

wherein
Z is O, NR¹, or a bond; each R^{N} is independently H or C₁₋₆alkyl; R¹ is C₅₋₈carbocyclyl optionally substituted with C₁₋₆alkylene-C₆₋₁₀aryl, or 5- to 8-membered N-heterocycle, wherein the ring nitrogen is optionally substituted with COO—C₁₋₆alkyl; R² is C₁₋₆alkyl, C₁₋₆alkylene-C₅₋₈carbocyclyl, 4-10 membered heterocyclyl having 1-3 ring heteroatoms selected from N, O, and S, C₁₋₆alkylene-C₆₋₁₀aryl, or C₀₋₆alkylene-5-10 membered heteroaryl having 1-3 ring heteroatoms selected from N, O, and S, wherein C₁₋₆alkylene is optionally substituted with 1-3 R⁷, and the carbocyclyl, heterocyclyl, aryl, and heteroaryl is optionally substituted with 1-2 substituents independently selected from halo, C₁₋₆alkoxy, C₁₋₆alkyl, C₁₋₆haloalkyl, C₁₋₆alkylene-C₆₋₁₀aryl, O—C₁₋₆alkylene-C₆₋₁₀aryl, and CO₂C₁₋₆alkyl; R³ is C₁₋₆alkyl, C₂₋₆ alkenyl, C₂₋₆alkynyl, C₁₋₆alkylene-C₅₋₈carbocyclyl, C₀₋₆alkylene-C₆₋₁₀aryl optionally substituted with 1-2 halo, or an amino acid side chain; each R⁴ is independently halo, OH, CN, C₁₋₆alkyl, C₁₋₆haloalkyl, C₁₋₆alkyl-OH, C₂₋₆alkenyl, C₂₋₆alkynyl, C₁₋₆alkoxy, C₃₋₆alkyloxyalkyl, oxo (═O), NR^{A}SO₂R^{B}, SO₂NR^{A}R^{B}, COOR^{A}, C₀₋₄alkylene-C₆₋₁₀aryl, C₀₋₄alkylene-(5-12 membered heteroaryl having 1-3 ring heteroatoms selected from N, O, and S), or C₀₋₄alkylene-(4-12 membered heterocycle having 1-3 ring heteroatoms selected from N, O, and S); and the aryl, heteroaryl, and heterocycle is optionally substituted with 1-2 substituents independently selected from halo, C₁₋₆alkyl, and COO—C₁₋₆alkyl, or two R⁴ with the carbon or carbons to which they are attached combine to form a spiro or fused 3-12 membered carbocyclic or heterocylic ring having 1-3 ring heteroatoms selected from N, O, and S, which is optionally substituted with 1-2 substituents independently selected from halo, C₁₋₆alkyl, C₁₋₆alkylene-O—C₁₋₆alkyl, C(O)—C₁₋₆alkyl, SO₂—C₁₋₆alkyl, C(O)—C₁₋₆alkyl, and COO—C₁₋₆alkyl; R⁵ is C₁₋₆alkylene-OH, C₁₋₆alkylene-OH substituted with PO(OCH₂CH₂)₂, C₁₋₆alkylene-OH substituted with SO₃H, —[C(O)]₁₋₂-(4-8 membered heterocycle having 1-3 ring heteroatoms selected from N, O, and S), —[C(O)]₁₋₂—NR^{N}R^{N}, C(O)—Y—H, or —[C(O)]₁₋₂—NR^{N}—Y—X-A, wherein A is H, C₃₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl; Y is a bond, C₁₋₆alkylene, C₁₋₆alkylene-O—C₁₋₆alkylene, or C₁₋₆alkenylene, wherein C₁₋₆alkylene and C₁₋₆alkenylene are optionally substituted with 1-3 substituents independently selected from halo, OH, NR^{N}R^{N}, and C₁₋₆alkoxy; X is a bond, NR^{N}R^{N}, C(O), SO₂, or OC(O); each R⁶ is independently H, C₁₋₆alkylene-OH, C₁₋₆alkylene-OH substituted with PO(OCH₂CH₂)₂, C₁₋₆alkylene-OH substituted with SO₃H, CHO, or C(O)-(4-8 membered heterocycle having 1-3 ring heteroatoms selected from N, O, and S); each R⁷ is independently halo, C₁₋₆haloalkyl, C₂₋₆alkenyl, C₃₋₅carbocyclyl, or C₀₋₆alkylene-C₆₋₁₀aryl, and C₆₋₁₀aryl is optionally substituted with 1-2 halo, or two R⁷ with the carbon or carbons to which they are attached combine to form a spiro or fused C₃₋₆carbocyclyl ring; R^{A} and R^{B} are each independently H, C₁₋₆alkyl, C₁₋₆haloalkyl, C₁₋₆alkoxy, C₂₋₆alkenyl, C₂₋₆alkynyl, C₃₋₆cycloalkyl, C₀₋₆alkylene-C₆₋₁₀aryl, C₀₋₆alkylene-5-8 membered heteroaryl having 1-3 ring heteroatoms selected from N, O, and S; n is 0-3; m is 0-5; and o is 0-5. In some embodiments, Z is 0 or NR¹; each R^{N} is independently H or C₁₋₆alkyl; R¹ is C₅₋₈carbocyclyl optionally substituted with C₁₋₆alkylene-C₆₋₁₀aryl, or 5- to 8-membered N-heterocycle, wherein the ring nitrogen is optionally substituted with COO—C₁₋₆alkyl; R² is C₁₋₆alkyl, C₁₋₆alkylene-C₅₋₈carbocyclyl, C₁₋₆alkylene-C₆₋₁₀aryl, or C₁₋₆alkylene-5-10 membered heteroaryl having 1-3 ring heteroatoms selected from N, O, and S, wherein C₁₋₆alkylene is optionally substituted with 1-2 substituents independently selected from C₁₋₆haloalkyl and C₂₋₆alkenyl, and the carbocyclyl, aryl, and heteroaryl is optionally substituted with 1-2 substituents independently selected from halo, C₁₋₆alkoxy, C₁₋₆alkyl, C₁₋₆haloalkyl, and CO₂C₁₋₆alkyl; R³ is C₁₋₆alkyl, C₂₋₆ alkenyl, C₂₋₆ alkynyl, C₁₋₆alkylene-C₅₋₈carbocyclyl, C₀₋₆alkylene-C₆₋₁₀aryl, or an amino acid side chain; each R⁴ is independently halo, OH, CN, C₁₋₆alkyl, C₁₋₆haloalkyl, C₁₋₆alkyl-OH, C₂₋₆alkenyl, C₂₋₆alkynyl, C₁₋₆alkoxy, C₃₋₆alkyloxyalkyl, oxo (═O), NR^{A}SO₂R^{B}, SO₂NR^{A}R^{B}, COOR^{A}, C₀₋₄alkylene-C₆₋₁₀aryl, C₀₋₄alkylene-(5-12 membered heteroaryl having 1-3 ring heteroatoms selected from N, O, and S), or C₀₋₄alkylene-(4-12 membered heterocycle having 1-3 ring heteroatoms selected from N, O, and S); and the aryl, heteroaryl, and heterocycle is optionally substituted with 1-2 substituents independently selected from halo, C₁₋₆alkyl, and COO—C₁₋₆alkyl, or two R⁴ with the carbon or carbons to which they are attached combine to form a spiro or fused 5-12 membered carbocyclic or heterocylic ring having 1-3 ring heteroatoms selected from N, O, and S, which is optionally substituted with 1-2 substituents independently selected from halo, C₁₋₆alkyl, C(O)—C₁₋₆alkyl, and COO—C₁₋₆alkyl; R⁵ is C₁₋₆alkylene-OH, C₁₋₆alkylene-OH substituted with PO(OCH₂CH₂)₂, C₁₋₆alkylene-OH substituted with SO₃H, CHO, C(O)-(4-8 membered heterocycle having 1-3 ring heteroatoms selected from N, O, and S), CONR^{N}R^{N}, or C(O)—C(O)NR^{N}—Y—X-A, wherein A is C₅₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl; Y is C₁₋₆alkylene optionally substituted with 1-3 substituents independently selected from halo, OH, NR^{N}R^{N}, and C₁₋₆alkoxy; X is null, NR^{N}R^{N}, C(O), SO₂, or OC(O); each R⁶ is independently H, C₁₋₆alkylene-OH, C₁₋₆alkylene-OH substituted with PO(OCH₂CH₂)₂, C₁₋₆alkylene-OH substituted with SO₃H, CHO, or C(O)-(4-8 membered heterocycle having 1-3 ring heteroatoms selected from N, O, and S); R^{A} and R^{B} are each independently H, C₁₋₆alkyl, C₁₋₆haloalkyl, C₁₋₆alkoxy, C₂₋₆alkenyl, C₂₋₆alkynyl, C₃₋₆cycloalkyl, C₀₋₆alkylene-C₆₋₁₀aryl, C₀₋₆alkylene-5-8 membered heteroaryl having 1-3 ring heteroatoms selected from N, O, and S; n is 0-3; m is 0-5; and o is 0-5.

Further provided are methods of administering to a biological sample or patient a safe and effective amount of a compound as disclosed herein, e.g., as represented by Formula I or a compound of Table A, B, or C.

Also provided herein are methods of reducing the amount of virus in a biological sample or in a patient by administering to said biological sample or patient an effective amount of a compound as disclosed herein, e.g., as represented by Formula I or a compound of Table A, B, or C.

Further provided are methods of treating or preventing a viral infection in a patient, comprising administering to said patient an effective amount of a compound as disclosed herein, e.g., as represented by Formula I or a compound of Table A, B, or C.

Also provided are pharmaceutical compositions comprising a compound as disclosed herein, e.g., as represented by Formula I or a compound of Table A, B, or C, or a pharmaceutically acceptable salt thereof, and a pharmaceutically acceptable excipient, carrier, adjuvant or vehicle.

Also provided are uses of a compound described herein for inhibiting or reducing the replication of virus in a biological sample or patient, for reducing the amount of virus in a biological sample or patient, or for treating a viral infection in a patient.

Further provided herein are uses of a compound described herein for the manufacture of a medicament for treating a viral infection in a patient, for reducing the amount of virus in a biological sample or in a patient, or for inhibiting the replication of virus in a biological sample or patient.

### DETAILED DESCRIPTION

Provided herein are compounds, and their use in treating or preventing a viral infection (e.g., a norovirus or coronavirus infection). Also provided are uses of the compounds described herein, or pharmaceutically acceptable salts thereof, or pharmaceutically acceptable compositions comprising such a compound or a pharmaceutically acceptable salt thereof, for inhibiting the replication of viruses in a biological sample or in a patient, for reducing the amount of viruses (reducing viral titer) in a biological sample or in a patient, and for treating a viral infection in a patient.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

Unless otherwise indicated, all tautomeric forms of the compounds of the disclosure are within the scope of the disclosure.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this disclosure. Such compounds are useful, for example, as analytical tools or probes in biological assays. Such compounds, especially deuterium analogs, can also be therapeutically useful.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

### Compounds

Provided herein are compounds of Formula (I), and pharmaceutically acceptable salts thereof:

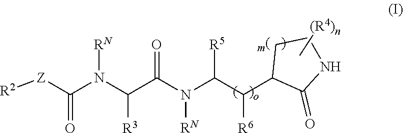

wherein

Z is O, NR¹, or a bond;

each R^{N} is independently H or C₁₋₆alkyl;

R¹ is C₅₋₈carbocyclyl optionally substituted with C₁₋₆alkylene-C₆₋₁₀aryl, or 5- to 8-membered N-heterocycle, wherein the ring nitrogen is optionally substituted with COO—C₁₋₆alkyl;

R² is C₁₋₆alkyl, C₁₋₆alkylene-C₅₋₈carbocyclyl, 4-10 membered heterocyclyl having 1-3 ring heteroatoms selected from N, O, and S, C₁₋₆alkylene-C₆₋₁₀aryl, or C₀₋₆alkylene-5-10 membered heteroaryl having 1-3 ring heteroatoms selected from N, O, and S, wherein C₁₋₆alkylene is optionally substituted with 1-3 R⁷, and the carbocyclyl, heterocyclyl, aryl, and heteroaryl is optionally substituted with 1-2 substituents independently selected from halo, C₁₋₆alkoxy, C₁₋₆alkyl, C₁₋₆haloalkyl, C₁₋₆alkylene-C₆₋₁₀aryl, 0-C₁₋₆alkylene-C₆₋₁₀aryl, and CO₂C₁₋₆alkyl;

R³ is C₁₋₆alkyl, C₂₋₆alkenyl, C₂₋₆alkynyl, C₁₋₆alkylene-C₅₋₈carbocyclyl, C₀₋₆alkylene-C₆₋₁₀aryl optionally substituted with 1-2 halo, or an amino acid side chain;

each R⁴ is independently halo, OH, CN, C₁₋₆alkyl, C₁₋₆haloalkyl, C₁₋₆alkyl-OH, C₂₋₆alkenyl, C₂₋₆alkynyl, C₁₋₆alkoxy, C₃₋₆alkyloxyalkyl, oxo (═O), NR^{A}SO₂R³, SO₂NR^{A}R^{B}, COOR^{A}, C₀₋₄alkylene-C₆₋₁₀aryl, C₀₋₄alkylene-(5-12 membered heteroaryl having 1-3 ring heteroatoms selected from N, O, and S), or C₀₋₄alkylene-(4-12 membered heterocycle having 1-3 ring heteroatoms selected from N, O, and S); and the aryl, heteroaryl, and heterocycle is optionally substituted with 1-2 substituents independently selected from halo, C₁₋₆alkyl, and COO—C₁₋₆alkyl, or

two R⁴ with the carbon or carbons to which they are attached combine to form a spiro or fused 3-12 membered carbocyclic or heterocylic ring having 1-3 ring heteroatoms selected from N, O, and S, which is optionally substituted with 1-2 substituents independently selected from halo, C₁₋₆alkyl, C₁₋₆alkylene-O—C₁₋₆alkyl, C(O)—C₁₋₆alkyl, SO₂—C₁₋₆alkyl, C(O)—C₁₋₆alkyl, and COO—C₁₋₆alkyl;

R⁵ is C₁₋₆alkylene-OH, C₁₋₆alkylene-OH substituted with PO(OCH₂CH₂)₂, C₁₋₆alkylene-OH substituted with SO₃H, —[C(O)]₁₋₂-(4-8 membered heterocycle having 1-3 ring heteroatoms selected from N, O, and S), —[C(O)]₁₋₂—NR^{N}R^{N}, C(O)—Y—H, or —[C(O)]₁₋₂—NR^{N}—Y—X-A, wherein A is H, C₃₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl;

Y is a bond, C₁₋₆alkylene, C₁₋₆alkylene-O—C₁₋₆alkylene, or C₁₋₆alkenylene, wherein C₁₋₆alkylene and C₁₋₆alkenylene are optionally substituted with 1-3 substituents independently selected from halo, OH, NR^{N}R^{N}, and C₁₋₆alkoxy;

X is a bond, NR^{N}R^{N}, C(O), SO₂, or OC(O);

each R⁶ is independently H, C₁₋₆alkylene-OH, C₁₋₆alkylene-OH substituted with PO(OCH₂CH₂)₂, C₁₋₆alkylene-OH substituted with SO₃H, CHO, or C(O)-(4-8 membered heterocycle having 1-3 ring heteroatoms selected from N, O, and S);

each R⁷ is independently halo, C₁₋₆haloalkyl, C₂₋₆alkenyl, C₃₋₅carbocyclyl, or C₀₋₆alkylene-C₆₋₁₀aryl, and C₆₋₁₀aryl is optionally substituted with 1-2 halo, or

two R⁷ with the carbon or carbons to which they are attached combine to form a spiro or fused C₃₋₆carbocyclyl ring;

R^{A} and R^{B} are each independently H, C₁₋₆alkyl, C₁₋₆haloalkyl, C₁₋₆alkoxy, C₂₋₆alkenyl, C₂₋₆alkynyl, C₃₋₆cycloalkyl, C₀₋₆alkylene-C₆₋₁₀aryl, C₀₋₆alkylene-5-8 membered heteroaryl having 1-3 ring heteroatoms selected from N, O, and S;

n is 0-3; m is 0-5; and o is 0-5.

In some embodiments, Z is 0 or NR¹;

each R^{N} is independently H or C₁₋₆alkyl;

R¹ is C₅₋₈carbocyclyl optionally substituted with C₁₋₆alkylene-C₆₋₁₀aryl, or 5- to 8-membered N-heterocycle, wherein the ring nitrogen is optionally substituted with COO—C₁₋₆alkyl;

R² is C₁₋₆alkyl, C₁₋₆alkylene-C₅₋₈carbocyclyl, C₁₋₆alkylene-C₆₋₁₀aryl, or C₁₋₆alkylene-5-10 membered heteroaryl having 1-3 ring heteroatoms selected from N, O, and S, wherein C₁₋₆alkylene is optionally substituted with 1-2 substituents independently selected from C₁₋₆haloalkyl and C₂₋₆alkenyl, and the carbocyclyl, aryl, and heteroaryl is optionally substituted with 1-2 substituents independently selected from halo, C₁₋₆alkoxy, C₁₋₆alkyl, C₁₋₆haloalkyl, and CO₂C₁₋₆alkyl;

R³ is C₁₋₆alkyl, C₂₋₆alkenyl, C₂₋₆alkynyl, C₁₋₆alkylene-C₅₋₈carbocyclyl, C₀₋₆alkylene-C₆₋₁₀aryl, or an amino acid side chain;

each R⁴ is independently halo, OH, CN, C₁₋₆alkyl, C₁₋₆haloalkyl, C₁₋₆alkyl-OH, C₂₋₆alkenyl, C₂₋₆alkynyl, C₁₋₆alkoxy, C₃₋₆alkyloxyalkyl, oxo (═O), NR^{A}SO₂R⁵, SO₂NR^{A}R^{B}, COOR^{A}, C₀₋₄alkylene-C₆₋₁₀aryl, C₀₋₄alkylene-(5-8 membered heteroaryl having 1-3 ring heteroatoms selected from N, O, and S), or C₀₋₄alkylene-(4-8 membered heterocycle having 1-3 ring heteroatoms selected from N, O, and S); and the aryl, heteroaryl, and heterocycle is optionally substituted with 1-2 substituents independently selected from halo, C₁₋₆alkyl, and COO—C₁₋₆alkyl, or

two R⁴ with the carbon or carbons to which they are attached combine to form a spiro or fused 5-12 membered carbocyclic or heterocylic ring having 1-3 ring heteroatoms selected from N, O, and S, which is optionally substituted with 1-2 substituents independently selected from halo, C₁₋₆alkyl, C(O)—C₁₋₆alkyl, and COO—C₁₋₆alkyl;

R⁵ is C₁₋₆alkylene-OH, C₁₋₆alkylene-OH substituted with PO(OCH₂CH₂)₂, C₁₋₆alkylene-OH substituted with SO₃H, CHO, C(O)-(4-8 membered heterocycle having 1-3 ring heteroatoms selected from N, O, and S), CONR^{N}R^{N}, or C(O)—C(O)NR^{N}—Y—X-A, wherein A is C₅₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl;

Y is C₁₋₆alkylene optionally substituted with 1-3 substituents independently selected from halo, OH, NR^{N}R^{N}, and C₁₋₆alkoxy;

X is null, NR^{N}R^{N}, C(O), SO₂, or OC(O);

each R⁶ is independently H, C₁₋₆alkylene-OH, C₁₋₆alkylene-OH substituted with PO(OCH₂CH₂)₂, C₁₋₆alkylene-OH substituted with SO₃H, CHO, or C(O)-(4-8 membered heterocycle having 1-3 ring heteroatoms selected from N, O, and S);

R^{A} and R^{B} are each independently H, C₁₋₆alkyl, C₁₋₆haloalkyl, C₁₋₆alkoxy, C₂₋₆alkenyl, C₂₋₆alkynyl, C₃₋₆cycloalkyl, C₀₋₆alkylene-C₆₋₁₀aryl, C₀₋₆alkylene-5-8 membered heteroaryl having 1-3 ring heteroatoms selected from N, O, and S;

n is 0-3; m is 0-5; and o is 0-5.

As used herein, the term “alkyl” or “alkylene” means a saturated straight or branched chain hydrocarbon. The term Cₙ means the alkyl group has “n” carbon atoms. For example, C₄alkyl refers to an alkyl group that has 4 carbon atoms. C₁₋₆alkyl refers to an alkyl group having a number of carbon atoms encompassing the entire range (i.e., 1 to 8 carbon atoms), as well as all subgroups (e.g., 1-6, 2-6, 1-5, 2-6, 1-4, 2-5, 1, 2, 3, 4, 5, and 6carbon atoms). Specific examples include, but are not limited to, methyl, ethyl, isopropyl, n-propyl, sec-butyl, and t-butyl.

As used herein, the terms “halogen” and “halo” mean F, Cl, Br, or I.

The term “carbocycle” (or “carbocyclyl”) refers to a non-aromatic monocyclic, fused, bridged or spiro ring system whose ring atoms are carbon and which can be saturated or have one or more units of unsaturation. The carbocycle can have five to eight ring carbon atoms. In some embodiments, the number of carbon atoms is 5 to 6. In some embodiments, the number of carbon atoms is 6. “Fused” bicyclic ring systems comprise two rings which share two adjoining ring atoms. Bridged bicyclic group comprise two rings which share three or four adjacent ring atoms. Spiro bicyclic ring systems share one ring atom. Cycloalkyl groups can include cycloalkenyl groups. Specific examples include, but are not limited to, cyclohexyl, cyclopentyl, cyclopropyl, and cyclobutyl. A carbocycle ring is unsubstituted or substituted as described herein.

The term “heterocycle” as used herein refers to a non-aromatic monocyclic, fused, spiro or bridged ring system which can be saturated or contain one or more units of unsaturation, having five to eight ring atoms in which one or more (e.g., one to three, or one, two, or three) ring atoms is a heteroatom selected from, N, S, and O. An “N-heterocyle” indicates that at least one of the ring heteroatoms is a nitrogen atom. In some embodiments, the heterocycle comprises 5-6 ring members. In some embodiments, the heterocycle comprises 5 ring members. In some embodiments, the heterocycle comprises 6 ring members. In some embodiments, the heterocycle is piperidinyl. Examples of heterocycles include, but are not limited to, quinuclidinyl, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydrothiophenyl, morpholino (including, for example, 3-morpholino, 4-morpholino), 2-thiomorpholino, 3-thiomorpholino, 4-thiomorpholino, 1-pyrrolidinyl, 2-pyrrolidinyl, 3-pyrrolidinyl, pyrrolidin-2-one, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolanyl, benzodithianyl, 3-(1-alkyl)benzimidazol-2-onyl, and 1,3-dihydro-imidazol-2-onyl. A heterocycle ring is unsubstituted or substituted as described herein.

The term “aryl” refers to aromatic ring groups have only carbon ring atoms (typically six to ten) and include monocyclic aromatic rings such as phenyl and fused polycyclic aromatic ring systems in which two or more carbocyclic aromatic rings are fused to one another. In some embodiments, aryl is phenyl. An aryl ring is unsubstituted or substituted as described herein.

The terms “heteroaryl” refers to a heterocycle that is aromatic, having five to eight members (e.g., 5 to 6 members), including monocyclic heteroaromatic rings and polycyclic aromatic rings in which a monocyclic aromatic ring is fused to one or more other aromatic ring. Heteroaryl groups have one or more ring (e.g., 1 to 4, 1 to 3, 1, 2, 3, or 4) heteroatoms selected from N, O, and S. Also included within the scope of the term “heteroaryl”, as it is used herein, is a group in which an aromatic ring is “fused” to one or more non-aromatic rings (carbocyclic or heterocyclic), where the radical or point of attachment is on the aromatic ring. Examples of heteroaryl groups include pyridyl, pyrazinyl, pyrimidinyl, pyridazinyl, imidazolyl, pyrrolyl, pyrazolyl, triazolyl, tetrazolyl, oxazolyl, isoxazolyl, oxadiazolyl, thiazolyl, isothiazolyl or thiadiazolyl including, for example, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, and 1,3,5-triazinyl. A heteroaryl ring is unsubstituted or substituted as described herein.

The term “amino acid side chain”, as used herein, refers to a side chain of an amino acid, e.g., methyl for alanine, isopropyl for valine, isobutyl for leucine, secbutyl for isoleucine. Contemplated amino acids for side chains include alanine, arginine, asparagine, aspartic acid, glutamine, glutamic acid, histidine, isoleucine, leucine, lysine, methionine, phenylalanine, serine, threonine, tryptophan, tyrosine, and valine.

As described herein, compounds of the disclosure may optionally be substituted with one or more substituents, such as illustrated generally, or as exemplified by particular classes, subclasses, and species of the disclosure. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position.

In some cases, at least one R^{N} is H. In some cases, at least one R^{N} is C₁₋₆alkyl. In some cases, each R^{N} is H. In some cases, at least one R^{N} is C₁₋₆alkyl, e.g., methyl. In some cases, each R^{N} is C₁₋₆alkyl, e.g., methyl.

In some cases, Z is O or NR¹. In some cases, Z is O or a bond. In some cases, Z is O. In some cases, Z is a bond. In various cases, Z is NR¹. In some cases, R¹ is C₅₋₈carbocyclyl optionally substituted with C₁₋₆alkylene-C₆₋₁₀aryl. In some cases, R¹ is unsubstituted C₅₋₈carbocyclyl. In some cases, R¹ is unsubstituted C₅₋₆carbocyclyl. In some cases, R¹ is cyclopentyl. In some cases, R¹ is cyclohexyl. In some cases, R¹ is C₅₋₈carbocyclyl substituted with C₁₋₆alkylene-C₆₋₁₀aryl. In some cases, R¹ is C₅₋₆carbocyclyl substituted with C₁₋₆alkylene-C₆₋₁₀aryl. In some cases, R¹ is C₅carbocyclyl substituted with C₁₋₆alkylene-C₆₋₁₀aryl. In some cases, R¹ is C₆carbocyclyl substituted with C₁₋₆alkylene-C₆₋₁₀aryl. In some cases, R¹ is C₅carbocyclyl substituted with C₁alkylene-C₆₋₁₀aryl. In some cases, R¹ is C₆carbocyclyl substituted with C₁alkylene-C₆₋₁₀aryl. In some cases, R¹ is C₅₋₆carbocyclyl substituted with benzyl. In some cases, R¹ is C₅carbocyclyl substituted with benzyl. In some cases, R¹ is C₆carbocyclyl substituted with benzyl.

In some cases, R¹ is 5- to 8-membered N-heterocycle, wherein the ring nitrogen is substituted with COO—C₁₋₆alkyl. In some cases, R¹ is 5- to 6-membered N-heterocycle, wherein the ring nitrogen is substituted with COO—C₁₋₆alkyl. In some cases, R¹ is 6-membered N-heterocycle, wherein the ring nitrogen is substituted with COO—C₁₋₆alkyl. In some cases, R¹ is 6-membered N-heterocycle, wherein the N-heterocycle ring nitrogen is substituted with COO-t-butyl.

In some cases, R² is C₁₋₆alkyl, C₁₋₆alkylene-C₅₋₈carbocyclyl, 4-10 membered heterocyclyl having 1-3 ring heteroatoms selected from N, O, and S, C₁₋₆alkylene-C₆₋₁₀aryl, 5-10 membered heteroaryl having 1-3 ring heteroatoms selected from N, O, and S, or C₁₋₆alkylene-5-10 membered heteroaryl having 1-3 ring heteroatoms selected from N, O, and S, wherein C₁₋₆alkylene is substituted with 1-3 R⁷. In some cases, R² is C₁₋₆alkyl. In some cases, R² is methyl. In some cases, R² is C₁₋₆alkylene-C₆₋₁₀aryl. In some cases, R² is C₁₋₆alkylene-C₆aryl. In some cases, R² is benzyl. In some cases, R² is C₁₋₆alkylene-C₅₋₈carbocyclyl. In some cases, R² is 4-10 membered heterocyclyl having 1-3 ring heteroatoms selected from N, O, and S. In some cases, R² is C₁₋₆alkylene-5-10 membered heteroaryl having 1-3 ring heteroatoms selected from N, O, and S. In some cases, the C₁₋₆alkylene of R² is substituted with 1-3 R⁷. In some cases, the C₁₋₆alkylene of R² is substituted with 1 R⁷. In some cases, the C₁₋₆alkylene of R² is substituted with 2 R⁷. In some cases, the C₁₋₆alkylene of R² is substituted with 3 R⁷. In some cases, the C₁₋₆alkylene of R² is unsubstituted. In some cases, the carbocyclyl, heterocyclyl, aryl, or heteroaryl of R² is substituted with 1-2 substituents independently selected from halo, C₁₋₆alkoxy, C₁₋₆alkyl, C₁₋₆haloalkyl, C₁₋₆alkylene-C₆₋₁₀aryl, O—C₁₋₆alkylene-C₆₋₁₀aryl, and CO₂C₁₋₆alkyl. In some cases, the carbocyclyl, heterocyclyl, aryl, or heteroaryl of R² is substituted with 1-2 substituents independently selected from halo, C₁₋₆alkoxy, C₁₋₆alkyl, C₁₋₆haloalkyl, and CO₂C₁₋₆alkyl. In some cases, the carbocyclyl, heterocyclyl, aryl, or heteroaryl of R² is unsubstituted.

In some cases, R³ is C₁₋₆alkyl. In some cases, R³ is C₄alkyl. In some cases, R² is

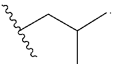 [SMILES: *CC(C)C]

In some cases, R³ is C₁₋₆alkylene-C₅₋₈carbocyclyl. In some cases, R³ is C₁₋₆alkylene-C₆carbocyclyl. In some cases, R³ is

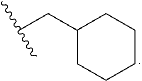 [SMILES: *CC1CCCCC1]

In some cases, R³ is C₂₋₆alkenyl or C₂₋₆alkynyl. In some cases, R³ is C₀₋₆alkylene-C₆₋₁₀aryl. In some cases, R³ is an amino acid side chain. In various cases, the amino acid side chain is methyl, isopropyl, isobutyl, sec-butyl, CH₂CH₂SCH₃, CH₂-indolyl, benzyl, CH₂OH, CH(OH)CH₃, CH₂SH, CH₂-(4-OH-phenyl), CH₂C(O)NH₂, CH₂CH₂C(O)NH₂, CH₂COOH, CH₂CH₂COOH, CH₂CH₂CH₂CH₂NH₂, CH₂CH₂CH₂NHC(NH)NH₂, or imidazolyl.

In the compounds disclosed herein, m is 0-5. In various cases, m is 0. In some cases, m is 1. In some cases, m is 2. In some cases, m is 3-5.

In some cases, n is 0. In some cases, n is 1, 2, or 3, or is 1 or 2. In some cases, each R⁴ is independently C₁₋₆alkyl, oxo (═O), C₀₋₄alkylene-C₆₋₁₀aryl, C₀₋₄alkylene-(5-12 membered heteroaryl having 1-3 ring heteroatoms selected from N, O, and S), or C₀₋₄alkylene-(4-12 membered heterocycle having 1-3 ring heteroatoms selected from N, O, and S); and the aryl, heteroaryl, and heterocycle is optionally substituted with 1-2 substitutents independently selected from halo, C₁₋₆alkyl, and COO—C₁₋₆alkyl. In some cases, at least one R⁴ is halo, OH, CN, C₁₋₆haloalkyl, C₁₋₆alkyl-OH, C₂₋₆alkenyl, C₂₋₆alkynyl, C₁₋₆alkoxy, C₃₋₆alkyloxyalkyl, NR^{A}SO₂R^{B}, SO₂NR^{A}R^{B}, or COOR^{A}. In some cases, two R⁴ on combine to form a spiro or fused 5-8 membered carbocycle or heterocylic ring having 1-3 ring heteroatoms selected from N, O, and S, which is optionally substituted with 1-2 substitutents independently selected from halo, C₁₋₆alkyl, C₁₋₆alkylene-O—C₁₋₆alkyl, C(O)—C₁₋₆alkyl, SO₂—C₁₋₆alkyl, C(O)—C₁₋₆alkyl, and COO—C₁₋₆alkyl. In some cases, two R⁴ on combine to form a spiro or fused 5-8 membered carbocycle or heterocylic ring having 1-3 ring heteroatoms selected from N, O, and S, which is optionally substituted with 1-2 substitutents independently selected from halo, C₁₋₆alkyl, C(O)—C₁₋₆alkyl, and COO—C₁₋₆alkyl. In some embodiments, n is 1 and R⁴ is C₁₋₆alkyl, oxo (═O), C₀₋₄alkylene-C₆₋₁₀aryl, C₀₋₄alkylene-(5-12 membered heteroaryl having 1-3 ring heteroatoms selected from N, O, and S), or C₀₋₄alkylene-(4-12 membered heterocycle having 1-3 ring heteroatoms selected from N, O, and S); and the aryl, heteroaryl, and heterocycle is optionally substituted with 1-2 substitutents independently selected from halo, C₁₋₆alkyl, and COO—C₁₋₆alkyl. In some cases, n is 2 and each R⁴ is independently C₁₋₆alkyl, oxo (═O), C₀₋₄alkylene-C₆₋₁₀aryl, C₀₋₄alkylene-(5-8 membered heteroaryl having 1-3 ring heteroatoms selected from N, O, and S), or C₀₋₄alkylene-(4-8 membered heterocycle having 1-3 ring heteroatoms selected from N, O, and S); and the aryl, heteroaryl, and heterocycle is optionally substituted with 1-2 substitutents independently selected from halo, C₁₋₆alkyl, and COO—C₁₋₆alkyl. In some cases, n is 2 and the two R⁴ with the carbon or carbons to which they are attached combine to form a spiro or fused 5-12 membered carbocycle or heterocylic ring having 1-3 ring heteroatoms selected from N, O, and S, which is optionally substituted with 1-2 substitutents independently selected from halo, C₁₋₆alkyl, C(O)—C₁₋₆alkyl, and COO—C₁₋₆alkyl. In some cases, the two R⁴ with the carbon to which they are attached form a spiro 5-12 membered carbocycle or heterocylic ring having 1-3 ring heteroatoms selected from N, O, and S, which is optionally substituted with 1-2 substitutents independently selected from halo, C₁₋₆alkyl, C(O)—C₁₋₆alkyl, and COO—C₁₋₆alkyl. In some cases, the two R⁴ with the carbons to which they are attached form a fused 5-12 membered carbocycle or heterocylic ring having 1-3 ring heteroatoms selected from N, O, and S, which is optionally substituted with 1-2 substitutents independently selected from halo, C₁₋₆alkyl, C(O)—C₁₋₆alkyl, and COO—C₁₋₆alkyl.

In the compounds disclosed herein, o is 0-5. In some cases, o is 0. In some cases, o is 1 or 2. In various embodiments, each R⁶ is H. In some case, at least one R⁶ is C₁₋₆alkylene-OH, C₁₋₆alkylene-OH substituted with PO(OCH₂CH₂)₂, C₁₋₆alkylene-OH substituted with SO₃H, CHO, or C(O)-(4-8 membered heterocycle having 1-3 ring heteroatoms selected from N, O, and S).

In some cases, R⁵ is C₁₋₆alkylene-OH, C₁₋₆alkylene-OH substituted with PO(OCH₂CH₂)₂, C₁₋₆alkylene-OH substituted with SO₃H, —[C(O)]₁₋₂-(4-8 membered heterocycle having 1-3 ring heteroatoms selected from N, O, and S), —[C(O)]₁₋₂—NR^{N}R^{N}, C(O)—Y—H, or C(O)—C(O)NR^{N}—Y—X-A, wherein A is H, C₃₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl. In some cases, R⁵ is C₁₋₆alkylene-OH, C₁₋₆alkylene-OH substituted with PO(OCH₂CH₂)₂, C₁₋₆alkylene-OH substituted with SO₃H, CHO, C(O)-(4-8 membered heterocycle having 1-3 ring heteroatoms selected from N, O, and S), or CONR^{N}R^{N}. In some cases, R⁵ is C₁₋₆alkylene-OH. In some cases, R⁵ is C₁₋₆alkylene-OH substituted with PO(OCH₂CH₂)₂. In some cases, R⁵ is C₁₋₆alkylene-OH substituted with SO₃H. In some cases, R⁵ is C₁alkylene-OH substituted with SO₃H. In some cases, R⁵ is C(O)—Y—H. In some cases, R⁵ is CHO. In some cases, R⁵ is C(O)-(4-8 membered heterocycle having 1-3 ring heteroatoms selected from N, O, and S). In some cases, R⁵ is C(O)—C(O)(4-8 membered heterocycle having 1-3 ring heteroatoms selected from N, O, and S). In some cases, R⁵ is CONR^{N}R^{N}. In some cases, R⁵ is C(O)—C(O)NR^{N}R^{N}.

In some cases, R⁵ is C(O)—C(O)NR^{N}—Y—X-A, wherein A is H, C₃₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl; Y is C₁₋₆alkylene optionally substituted with 1-3 substituents independently selected from halo, OH, NR^{N}R^{N}, and C₁₋₆alkoxy; and X is null, NR^{N}R^{N}, C(O), SO₂, or OC(O). In some cases, A is C₅₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl; Y is C₁₋₆alkylene optionally substituted with 1-3 substituents independently selected from halo, OH, NR^{N}R^{N}, and C₁₋₆alkoxy; and X is null, NR^{N}R^{N}, C(O), SO₂, or OC(O). In various cases, R^{N} is H. In various cases, Y is C₁₋₆alkylene. In some cases, Y is a bond, C₁₋₆alkylene, or C₁₋₆alkenylene, wherein C₁₋₆alkylene and C₁₋₆alkenylene are optionally substituted with 1-3 substituents independently selected from halo, OH, NR^{N}R^{N}, and C₁₋₆alkoxy. In some cases, Y is C₁₋₆alkylene substituted with 1-3 (or 1) substituent(s) independently selected from halo, OH, NR^{N}R^{N}, and C₁₋₆alkoxy. In some cases, X is a bond. In some cases, X is NR^{N}R^{N}, C(O), SO₂, or OC(O). In various cases, A is C₅₋₈carbocyclyl or C₆₋₁₀aryl, and optionally substituted with 1-2 substituents independently selected from halo, C₁₋₆alkyl, and COO—C₁₋₆alkyl. In some cases, A is 4-12-membered heterocycle having 1-3 ring heteroatoms selected from N, O, and S, or 5-8-membered heteroaryl having 1-3 ring heteroatoms selected from N, O, and S, optionally substituted with 1-2 substituents independently selected from halo, C₁₋₆alkyl, and COO—C₁₋₆alkyl. In some cases, A comprises pyridyl (e.g., 2-pyridyl).

In some cases, each R⁷ is independently halo, C₁₋₆haloalkyl, C₂₋₆ alkenyl, C₃₋₅ carbocyclyl, or C₀₋₆alkylene-C₆₋₁₀aryl, and C₆₋₁₀aryl is optionally substituted with 1-2 halo. In some cases, at least one R⁷ is halo. In some cases, at least one R⁷ is C₁₋₆haloalkyl. In some cases, at least one R⁷ is C₂₋₆alkenyl. In some cases, at least one R⁷ is C₃₋₅carbocyclyl. In some cases, at least one R⁷ is C₀₋₆alkylene-C₆₋₁₀aryl, and C₆₋₁₀aryl is optionally substituted with 1-2 halo. In some cases, at least one R⁷ is C₀₋₆alkylene-C₆₋₁₀aryl, and C₆₋₁₀aryl is substituted with 1-2 halo. In some cases, at least one R⁷ is C₀₋₆alkylene-C₆₋₁₀aryl, and C₆₋₁₀aryl is unsubstituted. In some cases, at least one R⁷ is phenyl optionally substituted with 1-2 halo. In some cases, at least one R⁷ is phenyl optionally substituted with 1 halo. In some cases, at least one R⁷ is chlorophenyl. In some cases, at least one R⁷ is phenyl. In some cases, two R⁷ with the carbon or carbons to which they are attached combine to form a spiro or fused C₃₋₆carbocyclyl ring. In some cases, two R⁷ with the carbon to which they are attached combine to form a spiro C₃₋₆carbocyclyl ring. In some cases, two R⁷ with the carbons to which they are attached combine to form a fused C₃₋₆carbocyclyl ring.

In some cases, R^{A} and R^{B} are each independently H, C₁₋₆alkyl, C₁₋₆haloalkyl, C₁₋₆alkoxy, C₂₋₆alkenyl, C₂₋₆alkynyl, C₃₋₆ cycloalkyl, C₀₋₆alkylene-C₆₋₁₀ aryl, C₀₋₆alkylene-5-8 membered heteroaryl having 1-3 ring heteroatoms selected from N, O, and S. In some cases, at least one of R^{A} and R^{B} is H. In some cases, each of R^{A} and R^{B} is H. In some cases, at least one of R^{A} and R^{B} is C₁₋₆alkyl. In some cases, R^{A} is C₁₋₆alkyl. In some cases, R^{A} is methyl. In some cases, R^{B} is C₁₋₆alkyl. In some cases, R^{B} is methyl.

Specific compounds contemplated include compounds in the following Tables. Compounds showing particular stereocenters indicate at least a relative stereoisomerism. Compounds having a chiral center without indication of a particular stereoisomerism indicate a mixture of stereocenters at that chiral center.

The compound can be a compound as listed in Table A, or a pharmaceutically acceptable salt thereof.

TABLE A

| | Structure |
|---|---|
| A1 | 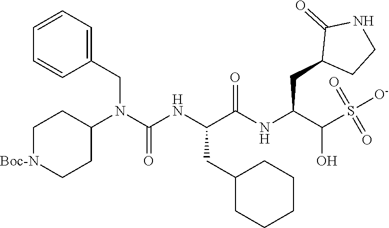 |
| A2 | 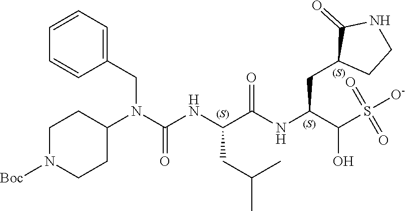 |
| A3 | 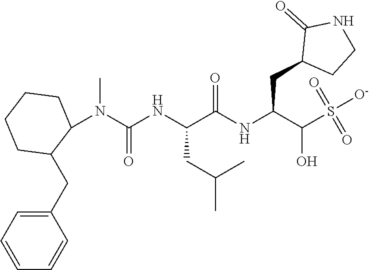 [SMILES: CC(C)C[C@H](NC(=O)N(C)C1CCCCC1Cc1ccccc1)C(=O)N[C@@H](C[C@@H]1CCNC1=O)C(O)S(=O)(=O)[O-]] |
| A4 | 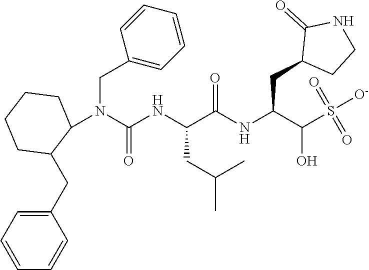 [SMILES: CC(C)C[C@H](NC(=O)N(Cc1ccccc1)C1CCCCC1Cc1ccccc1)C(=O)N[C@@H](C[C@@H]1CCNC1=O)C(O)S(=O)(=O)[O-]] |
| A5 | 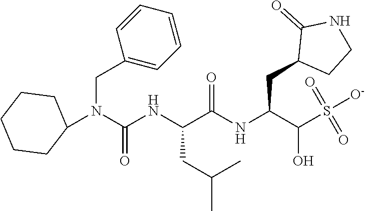 [SMILES: CC(C)C[C@H](NC(=O)N(Cc1ccccc1)C1CCCCC1)C(=O)N[C@@H](C[C@@H]1CCNC1=O)C(O)S(=O)(=O)[O-]] |
| A6 | 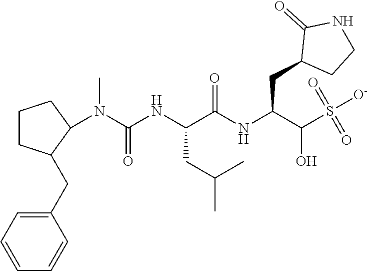 [SMILES: CC(C)C[C@H](NC(=O)N(C)C1CCCC1Cc1ccccc1)C(=O)N[C@@H](C[C@@H]1CCNC1=O)C(O)S(=O)(=O)[O-]] |
| A7 | 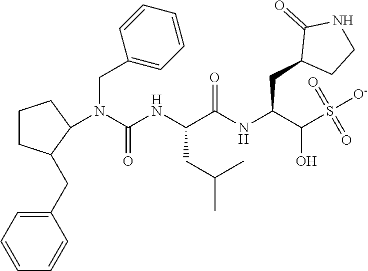 [SMILES: CC(C)C[C@H](NC(=O)N(Cc1ccccc1)C1CCCC1Cc1ccccc1)C(=O)N[C@@H](C[C@@H]1CCNC1=O)C(O)S(=O)(=O)[O-]] |
| A8 | 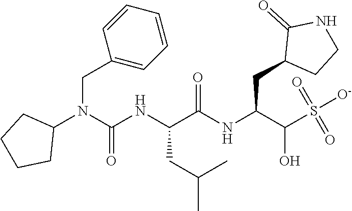 [SMILES: CC(C)C[C@H](NC(=O)N(Cc1ccccc1)C1CCCC1)C(=O)N[C@@H](C[C@@H]1CCNC1=O)C(O)S(=O)(=O)[O-]] |
| A9 | 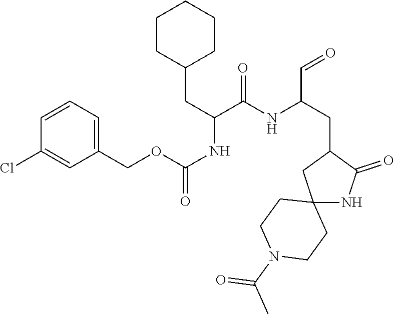 [SMILES: CC(=O)N1CCC2(CC1)CC(CC(C=O)NC(=O)C(CC1CCCCC1)NC(=O)OCc1cccc(Cl)c1)C(=O)N2] |
| A10 | 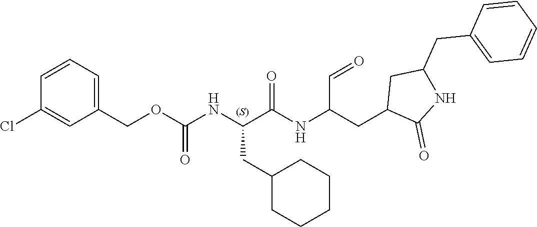 |

The compound can be a compound as listed in Table B, or a pharmaceutically acceptable salt thereof.

TABLE B

| | Structure |
|---|---|
| B1 | 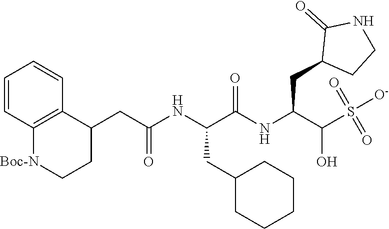 |

The compound can be a compound as listed in Table C, or a pharmaceutically acceptable salt thereof.

TABLE C

| | Structure |
|---|---|
| C1 | 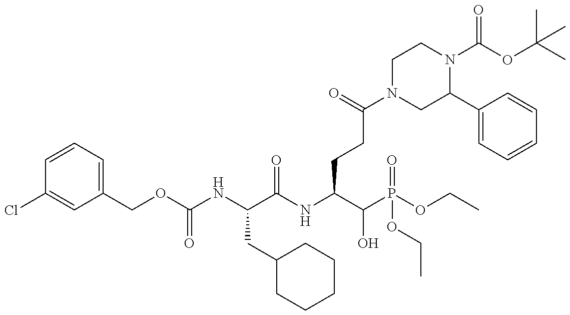 [SMILES: CCOP(=O)(OCC)C(O)[C@H](CCC(=O)N1CCN(C(=O)OC(C)(C)C)C(c2ccccc2)C1)NC(=O)[C@H](CC1CCCCC1)NC(=O)OCc1cccc(Cl)c1] |
| C2 | 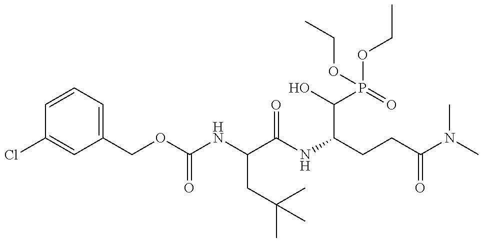 [SMILES: CCOP(=O)(OCC)C(O)[C@H](CCC(=O)N(C)C)NC(=O)C(CC(C)(C)C)NC(=O)OCc1cccc(Cl)c1] |
| C3 | 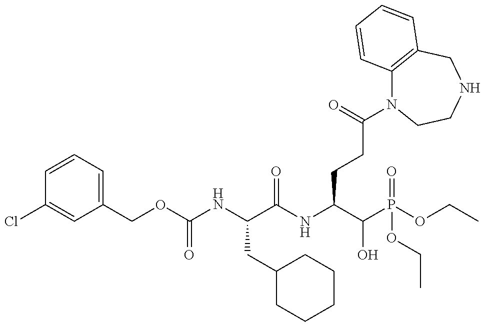 [SMILES: CCOP(=O)(OCC)C(O)[C@H](CCC(=O)N1CCNCc2ccccc21)NC(=O)[C@H](CC1CCCCC1)NC(=O)OCc1cccc(Cl)c1] |
| C4 | 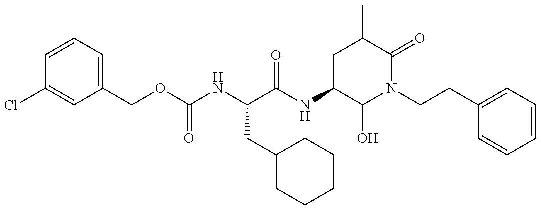 [SMILES: CC1C[C@H](NC(=O)[C@H](CC2CCCCC2)NC(=O)OCc2cccc(Cl)c2)C(O)N(CCc2ccccc2)C1=O] |
| C5 | 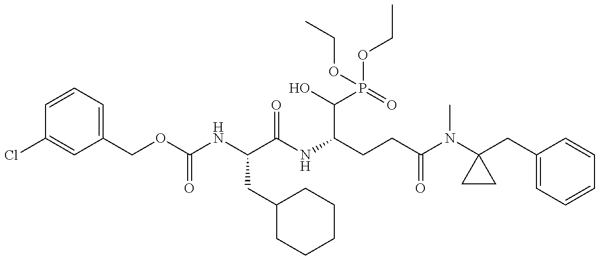 [SMILES: CCOP(=O)(OCC)C(O)[C@H](CCC(=O)N(C)C1(Cc2ccccc2)CC1)NC(=O)[C@H](CC1CCCCC1)NC(=O)OCc1cccc(Cl)c1] |
| C6 | 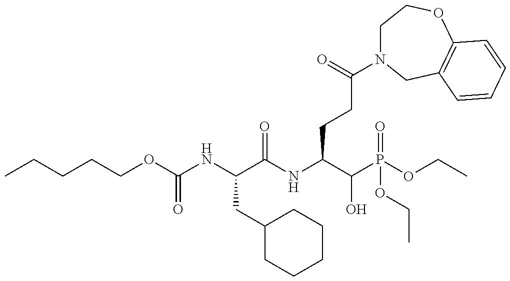 [SMILES: CCCCCOC(=O)N[C@@H](CC1CCCCC1)C(=O)N[C@@H](CCC(=O)N1CCOc2ccccc2C1)C(O)P(=O)(OCC)OCC] |
| C7 | 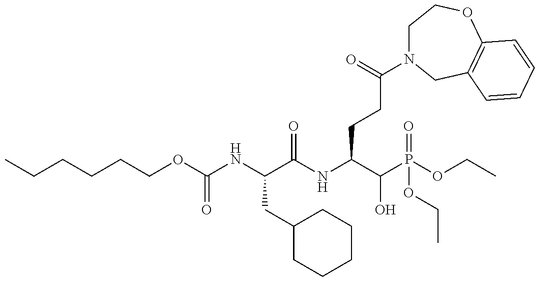 [SMILES: CCCCCCOC(=O)N[C@@H](CC1CCCCC1)C(=O)N[C@@H](CCC(=O)N1CCOc2ccccc2C1)C(O)P(=O)(OCC)OCC] |
| C8 | 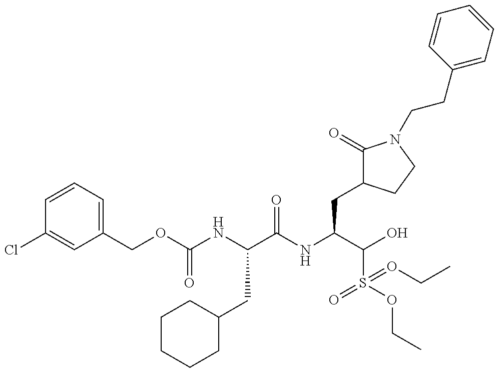 |
| C9 | 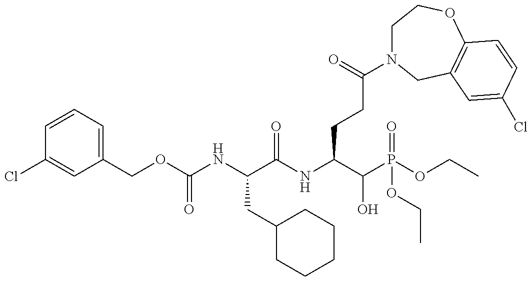 [SMILES: CCOP(=O)(OCC)C(O)[C@H](CCC(=O)N1CCOc2ccc(Cl)cc2C1)NC(=O)[C@H](CC1CCCCC1)NC(=O)OCc1cccc(Cl)c1] |
| C10 | 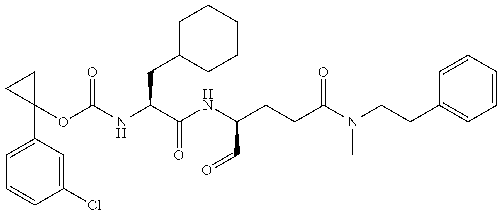 [SMILES: CN(CCc1ccccc1)C(=O)CC[C@@H](C=O)NC(=O)[C@H](CC1CCCCC1)NC(=O)OC1(c2cccc(Cl)c2)CC1] |
| C11 | 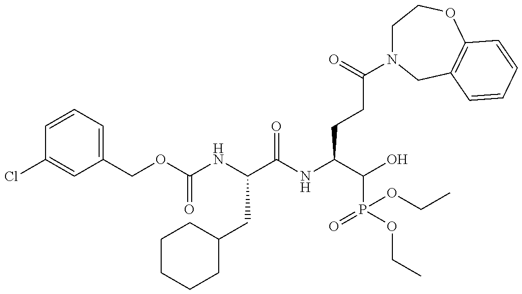 [SMILES: CCOP(=O)(OCC)C(O)[C@H](CCC(=O)N1CCOc2ccccc2C1)NC(=O)[C@H](CC1CCCCC1)NC(=O)OCc1cccc(Cl)c1] |
| C12 | 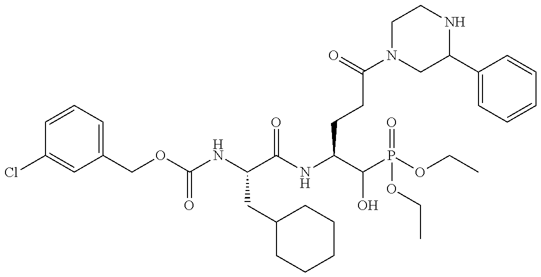 [SMILES: CCOP(=O)(OCC)C(O)[C@H](CCC(=O)N1CCNC(c2ccccc2)C1)NC(=O)[C@H](CC1CCCCC1)NC(=O)OCc1cccc(Cl)c1] |
| C13 | 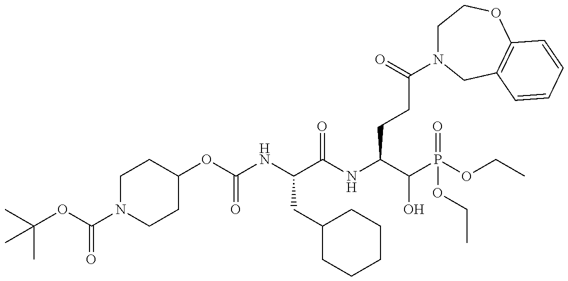 [SMILES: CCOP(=O)(OCC)C(O)[C@H](CCC(=O)N1CCOc2ccccc2C1)NC(=O)[C@H](CC1CCCCC1)NC(=O)OC1CCN(C(=O)OC(C)(C)C)CC1] |
| C14 | 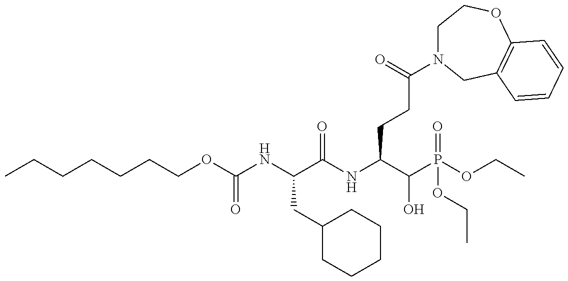 [SMILES: CCCCCCCOC(=O)N[C@@H](CC1CCCCC1)C(=O)N[C@@H](CCC(=O)N1CCOc2ccccc2C1)C(O)P(=O)(OCC)OCC] |
| C15 | 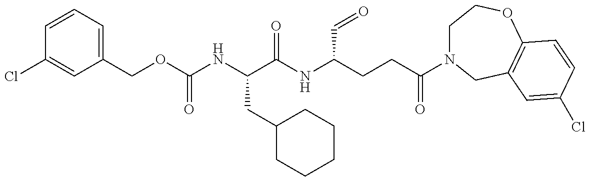 [SMILES: O=C[C@H](CCC(=O)N1CCOc2ccc(Cl)cc2C1)NC(=O)[C@H](CC1CCCCC1)NC(=O)OCc1cccc(Cl)c1] |
| C16 | 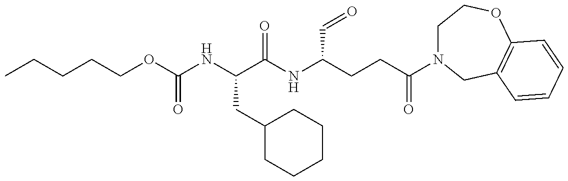 [SMILES: CCCCCOC(=O)N[C@@H](CC1CCCCC1)C(=O)N[C@H](C=O)CCC(=O)N1CCOc2ccccc2C1] |
| C17 | 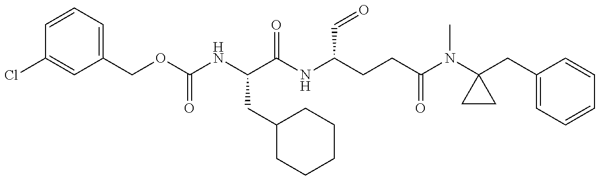 [SMILES: CN(C(=O)CC[C@@H](C=O)NC(=O)[C@H](CC1CCCCC1)NC(=O)OCc1cccc(Cl)c1)C1(Cc2ccccc2)CC1] |
| C18 | 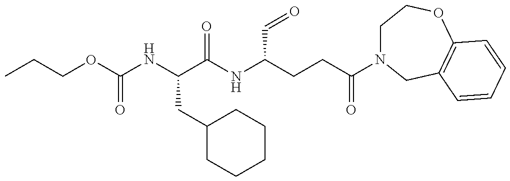 [SMILES: CCCOC(=O)N[C@@H](CC1CCCCC1)C(=O)N[C@H](C=O)CCC(=O)N1CCOc2ccccc2C1] |
| C19 | 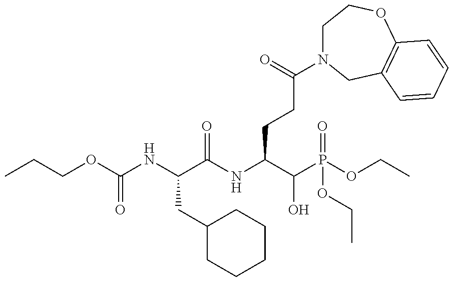 [SMILES: CCCOC(=O)N[C@@H](CC1CCCCC1)C(=O)N[C@@H](CCC(=O)N1CCOc2ccccc2C1)C(O)P(=O)(OCC)OCC] |
| C20 | 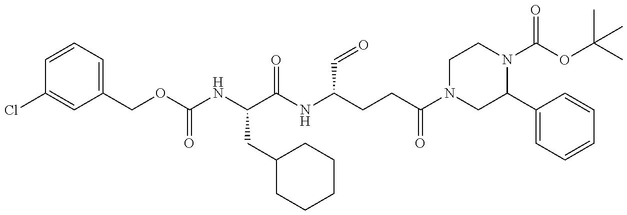 [SMILES: CC(C)(C)OC(=O)N1CCN(C(=O)CC[C@@H](C=O)NC(=O)[C@H](CC2CCCCC2)NC(=O)OCc2cccc(Cl)c2)CC1c1ccccc1] |
| C21 | 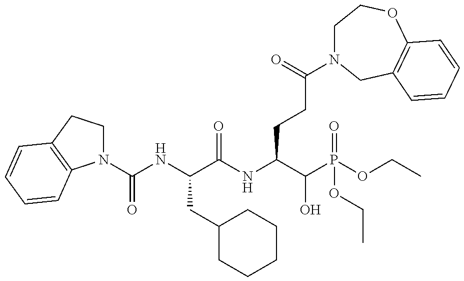 [SMILES: CCOP(=O)(OCC)C(O)[C@H](CCC(=O)N1CCOc2ccccc2C1)NC(=O)[C@H](CC1CCCCC1)NC(=O)N1CCc2ccccc21] |
| C22 | 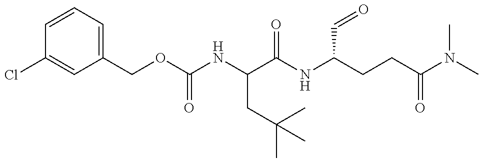 [SMILES: CN(C)C(=O)CC[C@@H](C=O)NC(=O)C(CC(C)(C)C)NC(=O)OCc1cccc(Cl)c1] |
| C23 | 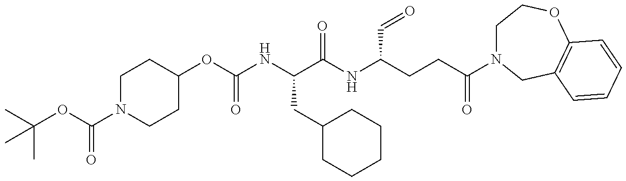 [SMILES: CC(C)(C)OC(=O)N1CCC(OC(=O)N[C@@H](CC2CCCCC2)C(=O)N[C@H](C=O)CCC(=O)N2CCOc3ccccc3C2)CC1] |
| C24 | 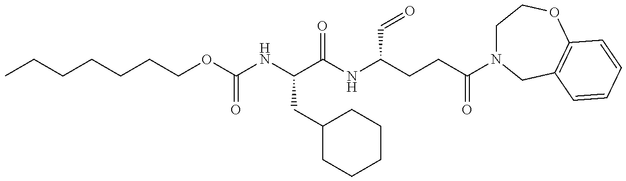 [SMILES: CCCCCCCOC(=O)N[C@@H](CC1CCCCC1)C(=O)N[C@H](C=O)CCC(=O)N1CCOc2ccccc2C1] |
| C25 | 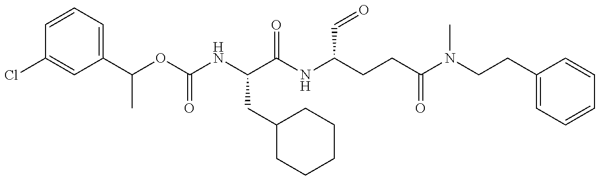 [SMILES: CC(OC(=O)N[C@@H](CC1CCCCC1)C(=O)N[C@H](C=O)CCC(=O)N(C)CCc1ccccc1)c1cccc(Cl)c1] |
| C26 | 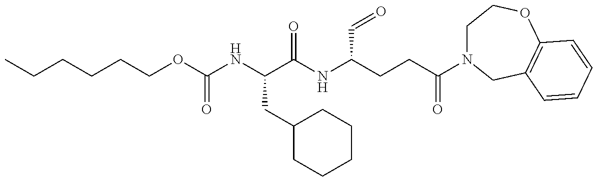 [SMILES: CCCCCCOC(=O)N[C@@H](CC1CCCCC1)C(=O)N[C@H](C=O)CCC(=O)N1CCOc2ccccc2C1] |
| C27 | 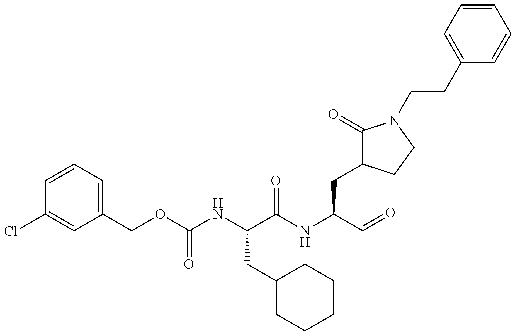 [SMILES: O=C[C@H](CC1CCN(CCc2ccccc2)C1=O)NC(=O)[C@H](CC1CCCCC1)NC(=O)OCc1cccc(Cl)c1] |
| C28 | 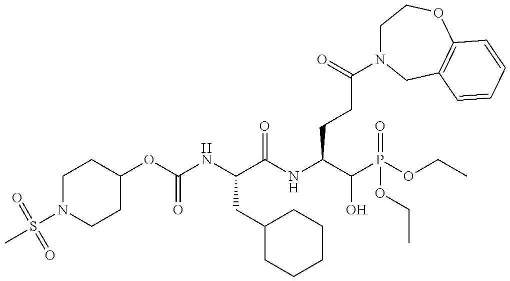 [SMILES: CCOP(=O)(OCC)C(O)[C@H](CCC(=O)N1CCOc2ccccc2C1)NC(=O)[C@H](CC1CCCCC1)NC(=O)OC1CCN(S(C)(=O)=O)CC1] |
| C29 | 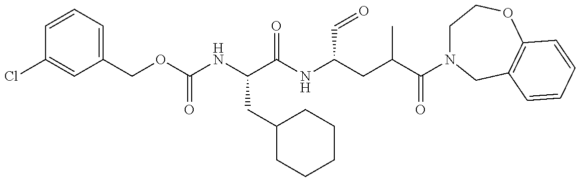 [SMILES: CC(C[C@@H](C=O)NC(=O)[C@H](CC1CCCCC1)NC(=O)OCc1cccc(Cl)c1)C(=O)N1CCOc2ccccc2C1] |
| C30 | 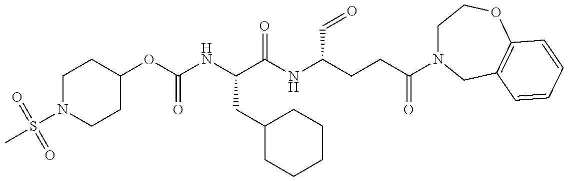 [SMILES: CS(=O)(=O)N1CCC(OC(=O)N[C@@H](CC2CCCCC2)C(=O)N[C@H](C=O)CCC(=O)N2CCOc3ccccc3C2)CC1] |
| C31 | 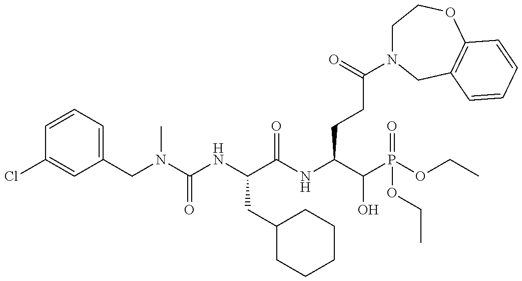 [SMILES: CCOP(=O)(OCC)C(O)[C@H](CCC(=O)N1CCOc2ccccc2C1)NC(=O)[C@H](CC1CCCCC1)NC(=O)N(C)Cc1cccc(Cl)c1] |
| C32 | 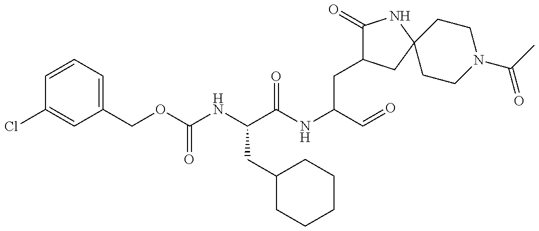 [SMILES: CC(=O)N1CCC2(CC1)CC(CC(C=O)NC(=O)[C@H](CC1CCCCC1)NC(=O)OCc1cccc(Cl)c1)C(=O)N2] |
| C33 | 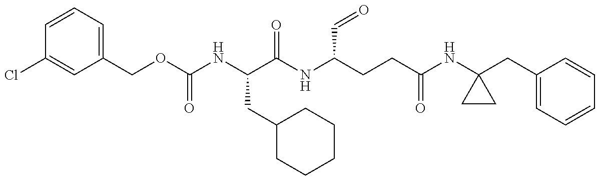 [SMILES: O=C[C@H](CCC(=O)NC1(Cc2ccccc2)CC1)NC(=O)[C@H](CC1CCCCC1)NC(=O)OCc1cccc(Cl)c1] |
| C34 | 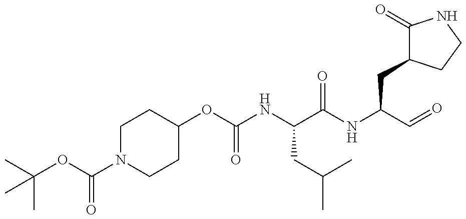 [SMILES: CC(C)C[C@H](NC(=O)OC1CCN(C(=O)OC(C)(C)C)CC1)C(=O)N[C@H](C=O)C[C@@H]1CCNC1=O] |
| C35 | 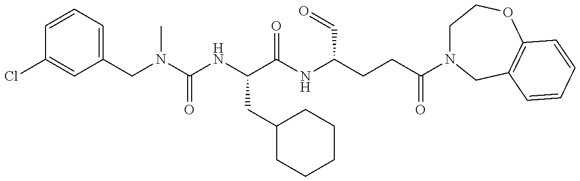 [SMILES: CN(Cc1cccc(Cl)c1)C(=O)N[C@@H](CC1CCCCC1)C(=O)N[C@H](C=O)CCC(=O)N1CCOc2ccccc2C1] |
| C36 | 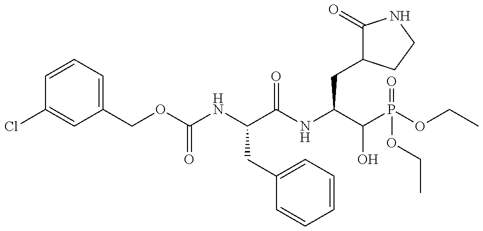 [SMILES: CCOP(=O)(OCC)C(O)[C@H](CC1CCNC1=O)NC(=O)[C@H](Cc1ccccc1)NC(=O)OCc1cccc(Cl)c1] |
| C37 | 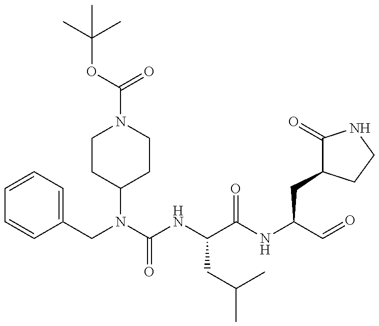 [SMILES: CC(C)C[C@H](NC(=O)N(Cc1ccccc1)C1CCN(C(=O)OC(C)(C)C)CC1)C(=O)N[C@H](C=O)C[C@@H]1CCNC1=O] |
| C38 | 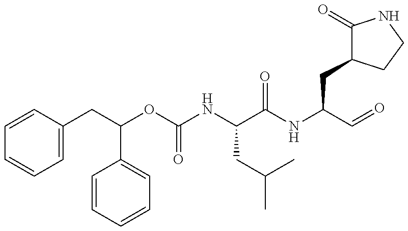 [SMILES: CC(C)C[C@H](NC(=O)OC(Cc1ccccc1)c1ccccc1)C(=O)N[C@H](C=O)C[C@@H]1CCNC1=O] |
| C39 | 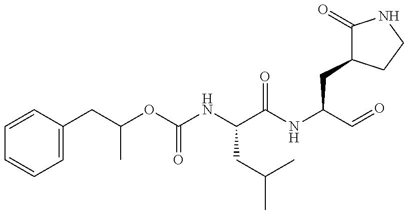 [SMILES: CC(C)C[C@H](NC(=O)OC(C)Cc1ccccc1)C(=O)N[C@H](C=O)C[C@@H]1CCNC1=O] |
| C40 | 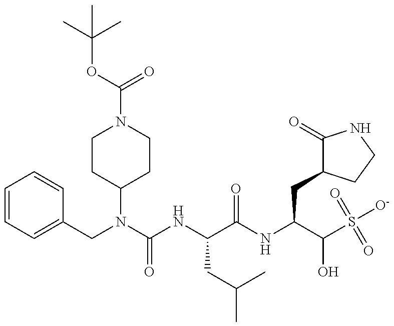 [SMILES: CC(C)C[C@H](NC(=O)N(Cc1ccccc1)C1CCN(C(=O)OC(C)(C)C)CC1)C(=O)N[C@@H](C[C@@H]1CCNC1=O)C(O)S(=O)(=O)[O-]] |
| C41 | 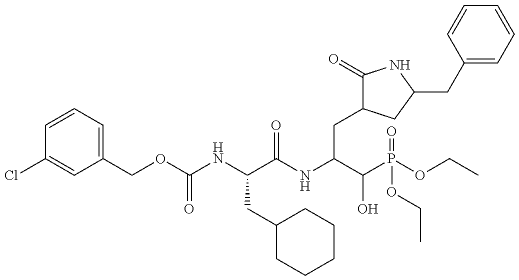 [SMILES: CCOP(=O)(OCC)C(O)C(CC1CC(Cc2ccccc2)NC1=O)NC(=O)[C@H](CC1CCCCC1)NC(=O)OCc1cccc(Cl)c1] |
| C42 | 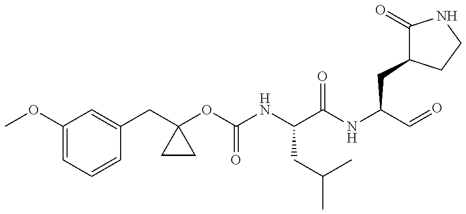 [SMILES: COc1cccc(CC2(OC(=O)N[C@@H](CC(C)C)C(=O)N[C@H](C=O)C[C@@H]3CCNC3=O)CC2)c1] |
| C43 | 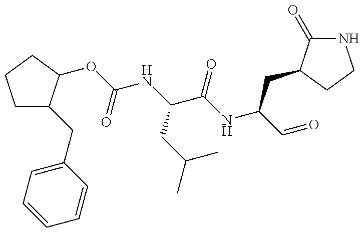 [SMILES: CC(C)C[C@H](NC(=O)OC1CCCC1Cc1ccccc1)C(=O)N[C@H](C=O)C[C@@H]1CCNC1=O] |
| C44 | 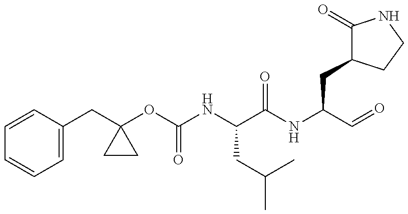 [SMILES: CC(C)C[C@H](NC(=O)OC1(Cc2ccccc2)CC1)C(=O)N[C@H](C=O)C[C@@H]1CCNC1=O] |
| C45 | 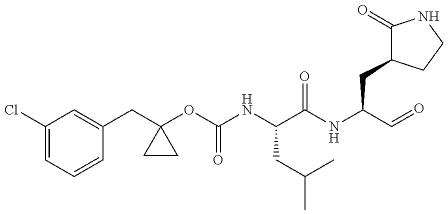 [SMILES: CC(C)C[C@H](NC(=O)OC1(Cc2cccc(Cl)c2)CC1)C(=O)N[C@H](C=O)C[C@@H]1CCNC1=O] |
| C46 | 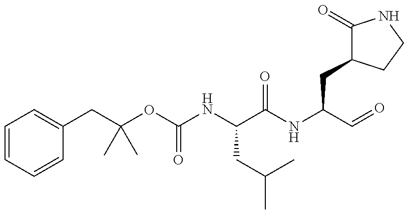 [SMILES: CC(C)C[C@H](NC(=O)OC(C)(C)Cc1ccccc1)C(=O)N[C@H](C=O)C[C@@H]1CCNC1=O] |
| C47 | 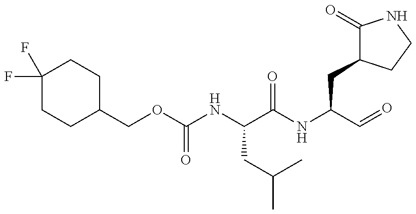 [SMILES: CC(C)C[C@H](NC(=O)OCC1CCC(F)(F)CC1)C(=O)N[C@H](C=O)C[C@@H]1CCNC1=O] |
| C48 | 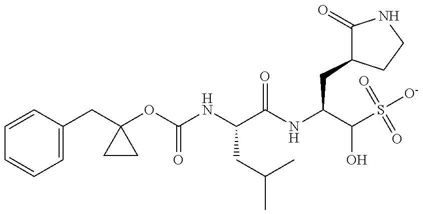 [SMILES: CC(C)C[C@H](NC(=O)OC1(Cc2ccccc2)CC1)C(=O)N[C@@H](C[C@@H]1CCNC1=O)C(O)S(=O)(=O)[O-]] |
| C49 | 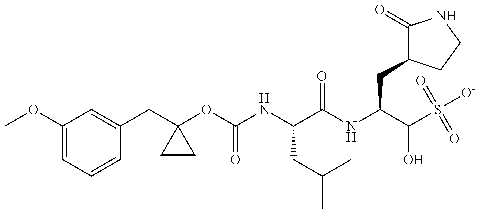 [SMILES: COc1cccc(CC2(OC(=O)N[C@@H](CC(C)C)C(=O)N[C@@H](C[C@@H]3CCNC3=O)C(O)S(=O)(=O)[O-])CC2)c1] |
| C50 | 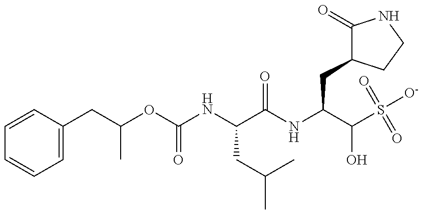 [SMILES: CC(C)C[C@H](NC(=O)OC(C)Cc1ccccc1)C(=O)N[C@@H](C[C@@H]1CCNC1=O)C(O)S(=O)(=O)[O-]] |
| C51 | 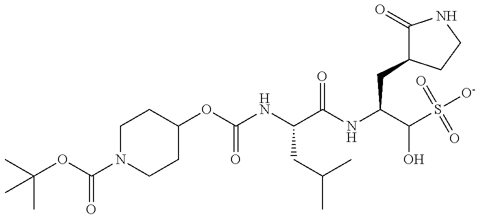 [SMILES: CC(C)C[C@H](NC(=O)OC1CCN(C(=O)OC(C)(C)C)CC1)C(=O)N[C@@H](C[C@@H]1CCNC1=O)C(O)S(=O)(=O)[O-]] |
| C52 | 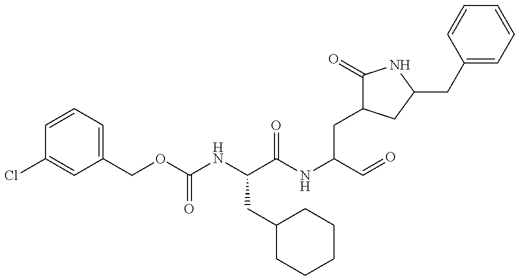 [SMILES: O=CC(CC1CC(Cc2ccccc2)NC1=O)NC(=O)[C@H](CC1CCCCC1)NC(=O)OCc1cccc(Cl)c1] |
| C53 | 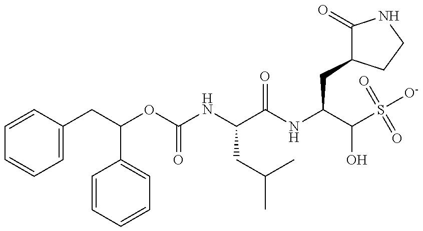 [SMILES: CC(C)C[C@H](NC(=O)OC(Cc1ccccc1)c1ccccc1)C(=O)N[C@@H](C[C@@H]1CCNC1=O)C(O)S(=O)(=O)[O-]] |
| C54 | 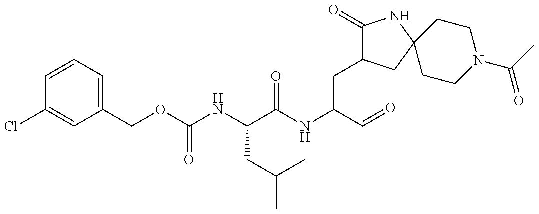 [SMILES: CC(=O)N1CCC2(CC1)CC(CC(C=O)NC(=O)[C@H](CC(C)C)NC(=O)OCc1cccc(Cl)c1)C(=O)N2] |
| C55 | 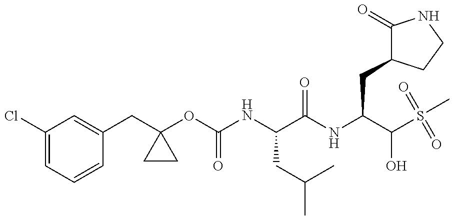 [SMILES: CC(C)C[C@H](NC(=O)OC1(Cc2cccc(Cl)c2)CC1)C(=O)N[C@@H](C[C@@H]1CCNC1=O)C(O)S(C)(=O)=O] |
| C56 | 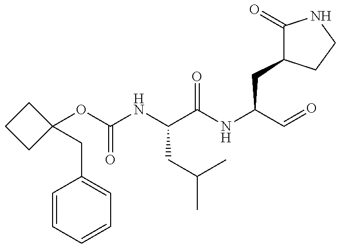 [SMILES: CC(C)C[C@H](NC(=O)OC1(Cc2ccccc2)CCC1)C(=O)N[C@H](C=O)C[C@@H]1CCNC1=O] |
| C57 | 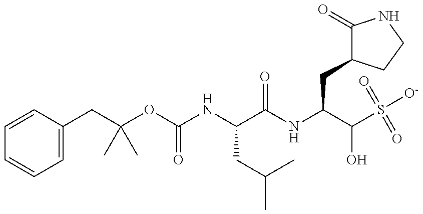 [SMILES: CC(C)C[C@H](NC(=O)OC(C)(C)Cc1ccccc1)C(=O)N[C@@H](C[C@@H]1CCNC1=O)C(O)S(=O)(=O)[O-]] |
| C58 | 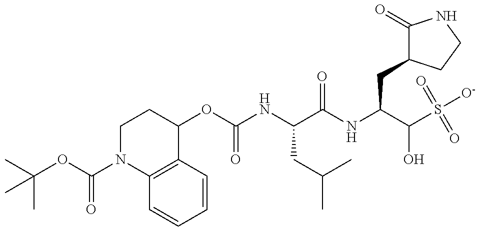 [SMILES: CC(C)C[C@H](NC(=O)OC1CCN(C(=O)OC(C)(C)C)c2ccccc21)C(=O)N[C@@H](C[C@@H]1CCNC1=O)C(O)S(=O)(=O)[O-]] |
| C59 | 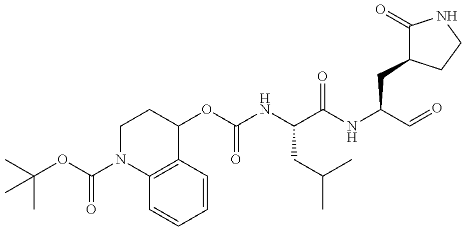 [SMILES: CC(C)C[C@H](NC(=O)OC1CCN(C(=O)OC(C)(C)C)c2ccccc21)C(=O)N[C@H](C=O)C[C@@H]1CCNC1=O] |
| C60 | 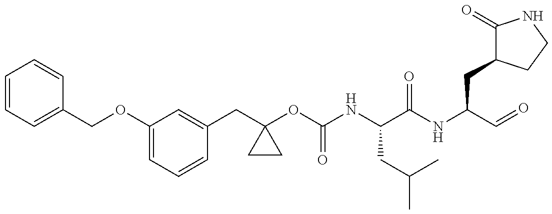 [SMILES: CC(C)C[C@H](NC(=O)OC1(Cc2cccc(OCc3ccccc3)c2)CC1)C(=O)N[C@H](C=O)C[C@@H]1CCNC1=O] |
| C61 | 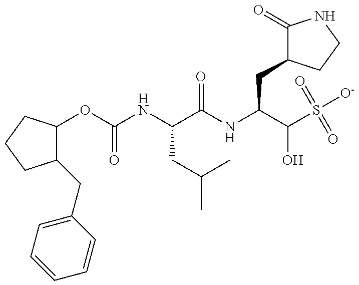 [SMILES: CC(C)C[C@H](NC(=O)OC1CCCC1Cc1ccccc1)C(=O)N[C@@H](C[C@@H]1CCNC1=O)C(O)S(=O)(=O)[O-]] |
| C62 | 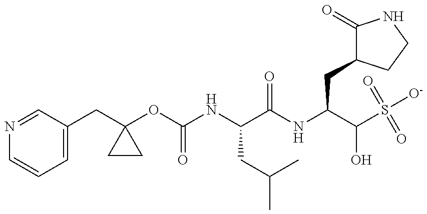 [SMILES: CC(C)C[C@H](NC(=O)OC1(Cc2cccnc2)CC1)C(=O)N[C@@H](C[C@@H]1CCNC1=O)C(O)S(=O)(=O)[O-]] |
| C63 | 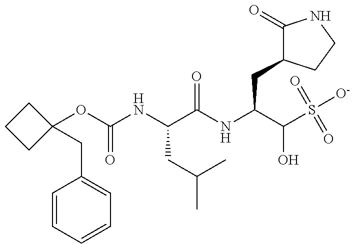 [SMILES: CC(C)C[C@H](NC(=O)OC1(Cc2ccccc2)CCC1)C(=O)N[C@@H](C[C@@H]1CCNC1=O)C(O)S(=O)(=O)[O-]] |
| C64 | 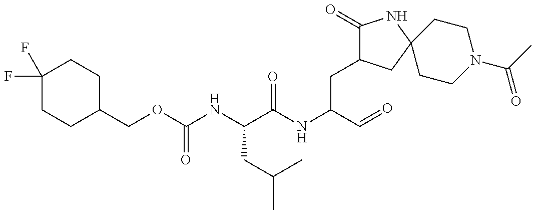 [SMILES: CC(=O)N1CCC2(CC1)CC(CC(C=O)NC(=O)[C@H](CC(C)C)NC(=O)OCC1CCC(F)(F)CC1)C(=O)N2] |
| C65 | 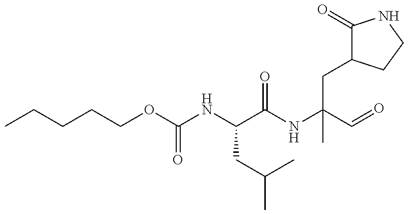 [SMILES: CCCCCOC(=O)N[C@@H](CC(C)C)C(=O)NC(C)(C=O)CC1CCNC1=O] |
| C66 | 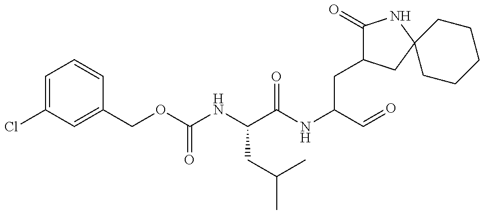 [SMILES: CC(C)C[C@H](NC(=O)OCc1cccc(Cl)c1)C(=O)NC(C=O)CC1CC2(CCCCC2)NC1=O] |
| C67 | 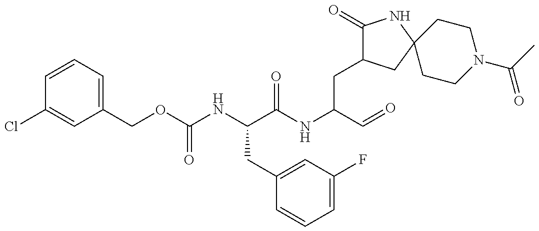 [SMILES: CC(=O)N1CCC2(CC1)CC(CC(C=O)NC(=O)[C@H](Cc1cccc(F)c1)NC(=O)OCc1cccc(Cl)c1)C(=O)N2] |
| C68 | 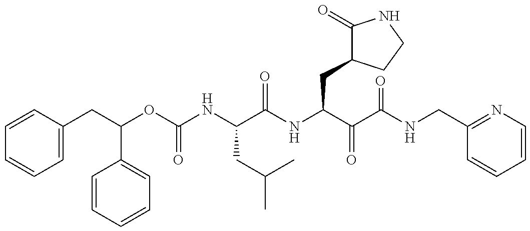 [SMILES: CC(C)C[C@H](NC(=O)OC(Cc1ccccc1)c1ccccc1)C(=O)N[C@@H](C[C@@H]1CCNC1=O)C(=O)C(=O)NCc1ccccn1] |
| C69 | 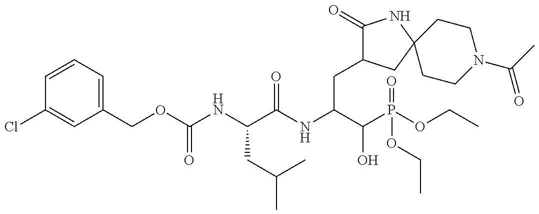 [SMILES: CCOP(=O)(OCC)C(O)C(CC1CC2(CCN(C(C)=O)CC2)NC1=O)NC(=O)[C@H](CC(C)C)NC(=O)OCc1cccc(Cl)c1] |
| C70 | 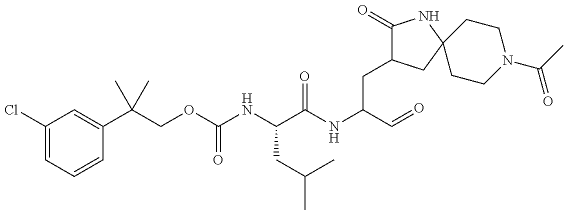 [SMILES: CC(=O)N1CCC2(CC1)CC(CC(C=O)NC(=O)[C@H](CC(C)C)NC(=O)OCC(C)(C)c1cccc(Cl)c1)C(=O)N2] |
| C71 | 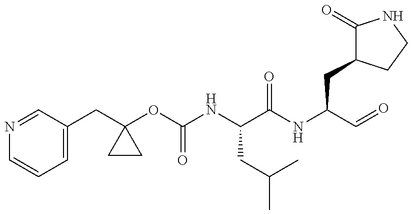 [SMILES: CC(C)C[C@H](NC(=O)OC1(Cc2cccnc2)CC1)C(=O)N[C@H](C=O)C[C@@H]1CCNC1=O] |
| C72 | 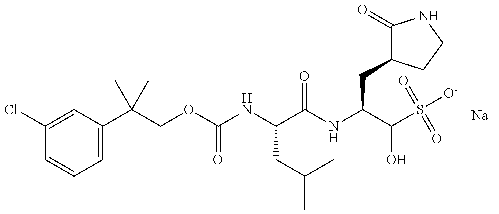 [SMILES: CC(C)C[C@H](NC(=O)OCC(C)(C)c1cccc(Cl)c1)C(=O)N[C@@H](C[C@@H]1CCNC1=O)C(O)S(=O)(=O)[O-] ; [Na+]] |
| C73 | 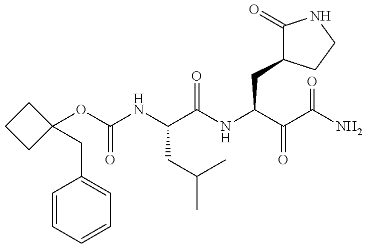 [SMILES: CC(C)C[C@H](NC(=O)OC1(Cc2ccccc2)CCC1)C(=O)N[C@@H](C[C@@H]1CCNC1=O)C(=O)C(N)=O] |
| C74 | 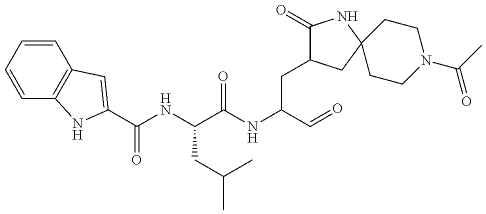 [SMILES: CC(=O)N1CCC2(CC1)CC(CC(C=O)NC(=O)[C@H](CC(C)C)NC(=O)c1cc3ccccc3[nH]1)C(=O)N2] |
| C75 | 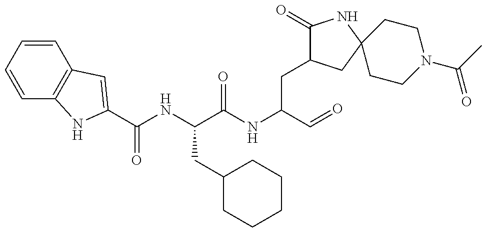 [SMILES: CC(=O)N1CCC2(CC1)CC(CC(C=O)NC(=O)[C@H](CC1CCCCC1)NC(=O)c1cc3ccccc3[nH]1)C(=O)N2] |
| C76 | 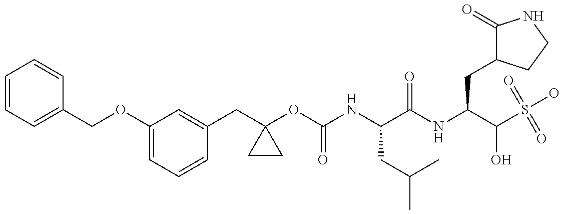 [SMILES: CC(C)C[C@H](NC(=O)OC1(Cc2cccc(OCc3ccccc3)c2)CC1)C(=O)N[C@@H](CC1CCNC1=O)C(O)S(=O)(=O)O] |
| C77 | 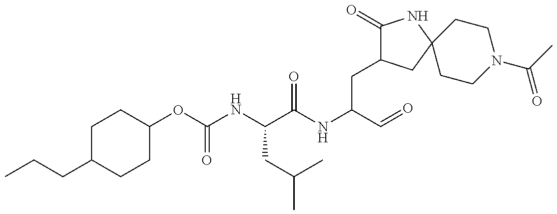 [SMILES: CCCC1CCC(OC(=O)N[C@@H](CC(C)C)C(=O)NC(C=O)CC2CC3(CCN(C(C)=O)CC3)NC2=O)CC1] |
| C78 | 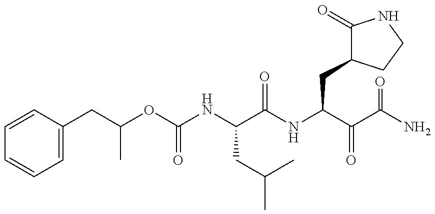 [SMILES: CC(C)C[C@H](NC(=O)OC(C)Cc1ccccc1)C(=O)N[C@@H](C[C@@H]1CCNC1=O)C(=O)C(N)=O] |
| C79 | 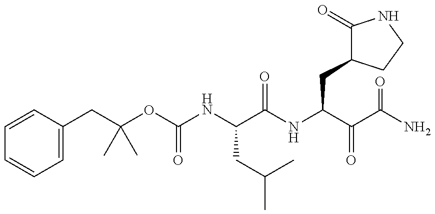 [SMILES: CC(C)C[C@H](NC(=O)OC(C)(C)Cc1ccccc1)C(=O)N[C@@H](C[C@@H]1CCNC1=O)C(=O)C(N)=O] |
| C80 | 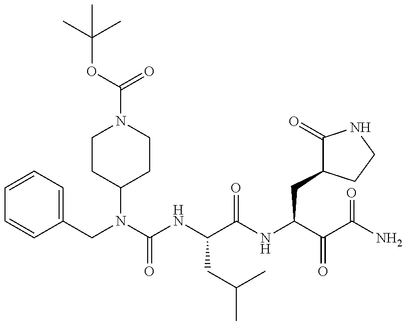 [SMILES: CC(C)C[C@H](NC(=O)N(Cc1ccccc1)C1CCN(C(=O)OC(C)(C)C)CC1)C(=O)N[C@@H](C[C@@H]1CCNC1=O)C(=O)C(N)=O] |
| C81 | 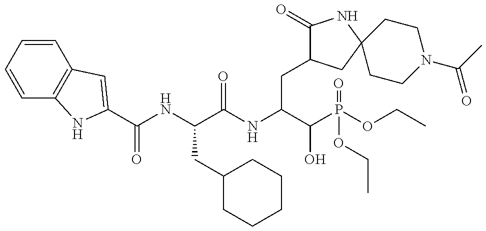 [SMILES: CCOP(=O)(OCC)C(O)C(CC1CC2(CCN(C(C)=O)CC2)NC1=O)NC(=O)[C@H](CC1CCCCC1)NC(=O)c1cc2ccccc2[nH]1] |
| C82 | 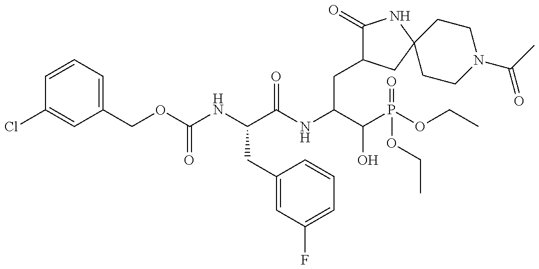 [SMILES: CCOP(=O)(OCC)C(O)C(CC1CC2(CCN(C(C)=O)CC2)NC1=O)NC(=O)[C@H](Cc1cccc(F)c1)NC(=O)OCc1cccc(Cl)c1] |
| C83 | 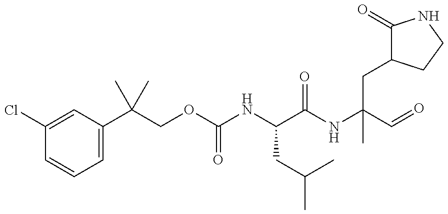 [SMILES: CC(C)C[C@H](NC(=O)OCC(C)(C)c1cccc(Cl)c1)C(=O)NC(C)(C=O)CC1CCNC1=O] |
| C84 | 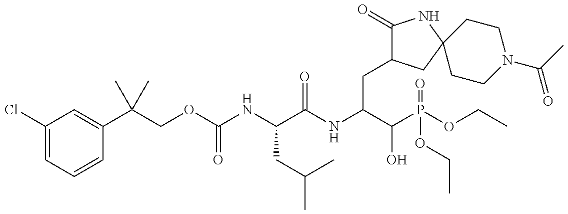 [SMILES: CCOP(=O)(OCC)C(O)C(CC1CC2(CCN(C(C)=O)CC2)NC1=O)NC(=O)[C@H](CC(C)C)NC(=O)OCC(C)(C)c1cccc(Cl)c1] |
| C85 | 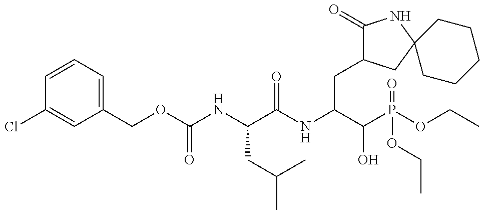 [SMILES: CCOP(=O)(OCC)C(O)C(CC1CC2(CCCCC2)NC1=O)NC(=O)[C@H](CC(C)C)NC(=O)OCc1cccc(Cl)c1] |
| C86 | 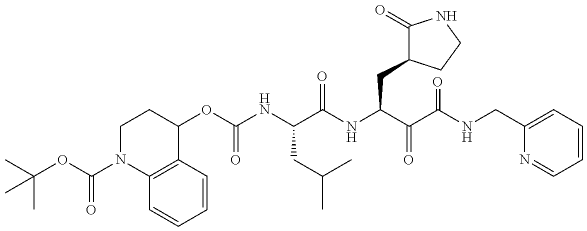 [SMILES: CC(C)C[C@H](NC(=O)OC1CCN(C(=O)OC(C)(C)C)c2ccccc21)C(=O)N[C@@H](C[C@@H]1CCNC1=O)C(=O)C(=O)NCc1ccccn1] |
| C87 | 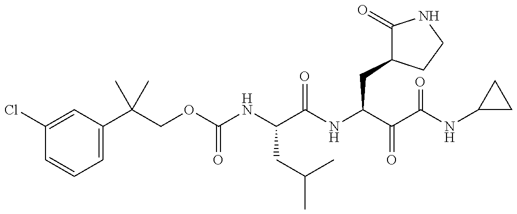 [SMILES: CC(C)C[C@H](NC(=O)OCC(C)(C)c1cccc(Cl)c1)C(=O)N[C@@H](C[C@@H]1CCNC1=O)C(=O)C(=O)NC1CC1] |
| C88 | 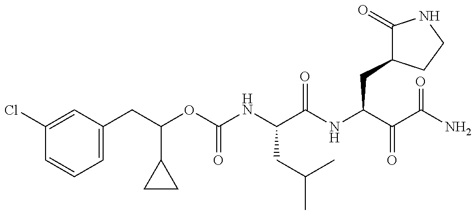 [SMILES: CC(C)C[C@H](NC(=O)OC(Cc1cccc(Cl)c1)C1CC1)C(=O)N[C@@H](C[C@@H]1CCNC1=O)C(=O)C(N)=O] |
| C89 | 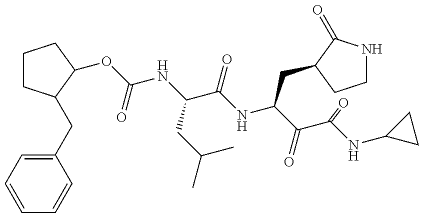 [SMILES: CC(C)C[C@H](NC(=O)OC1CCCC1Cc1ccccc1)C(=O)N[C@@H](C[C@@H]1CCNC1=O)C(=O)C(=O)NC1CC1] |
| C90 | 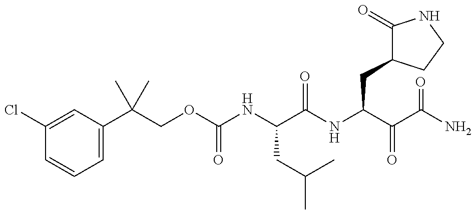 [SMILES: CC(C)C[C@H](NC(=O)OCC(C)(C)c1cccc(Cl)c1)C(=O)N[C@@H](C[C@@H]1CCNC1=O)C(=O)C(N)=O] |
| C91 | 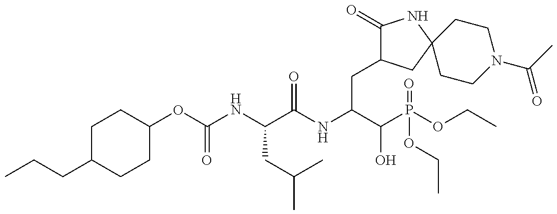 [SMILES: CCCC1CCC(OC(=O)N[C@@H](CC(C)C)C(=O)NC(CC2CC3(CCN(C(C)=O)CC3)NC2=O)C(O)P(=O)(OCC)OCC)CC1] |
| C92 | 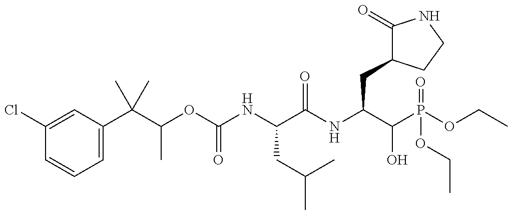 [SMILES: CCOP(=O)(OCC)C(O)[C@H](C[C@@H]1CCNC1=O)NC(=O)[C@H](CC(C)C)NC(=O)OC(C)C(C)(C)c1cccc(Cl)c1] |
| C93 | 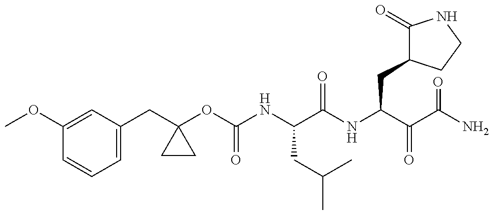 [SMILES: COc1cccc(CC2(OC(=O)N[C@@H](CC(C)C)C(=O)N[C@@H](C[C@@H]3CCNC3=O)C(=O)C(N)=O)CC2)c1] |
| C94 | 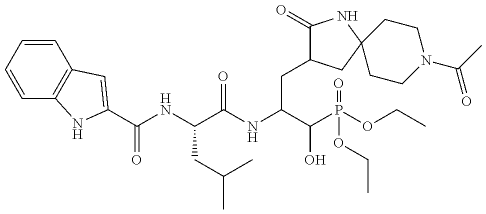 [SMILES: CCOP(=O)(OCC)C(O)C(CC1CC2(CCN(C(C)=O)CC2)NC1=O)NC(=O)[C@H](CC(C)C)NC(=O)c1cc2ccccc2[nH]1] |
| C95 | 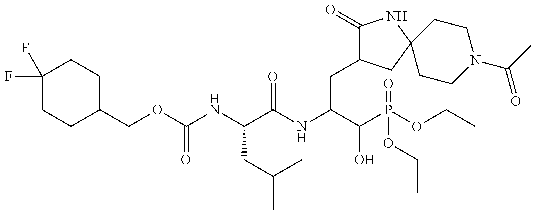 [SMILES: CCOP(=O)(OCC)C(O)C(CC1CC2(CCN(C(C)=O)CC2)NC1=O)NC(=O)[C@H](CC(C)C)NC(=O)OCC1CCC(F)(F)CC1] |
| C96 | 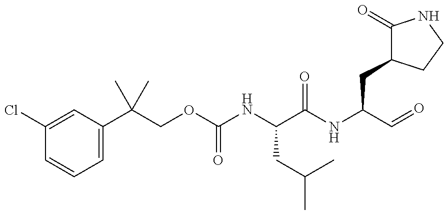 [SMILES: CC(C)C[C@H](NC(=O)OCC(C)(C)c1cccc(Cl)c1)C(=O)N[C@H](C=O)C[C@@H]1CCNC1=O] |
| C97 | 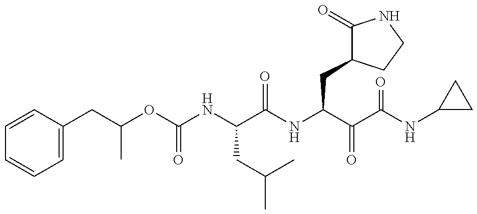 [SMILES: CC(C)C[C@H](NC(=O)OC(C)Cc1ccccc1)C(=O)N[C@@H](C[C@@H]1CCNC1=O)C(=O)C(=O)NC1CC1] |
| C98 | 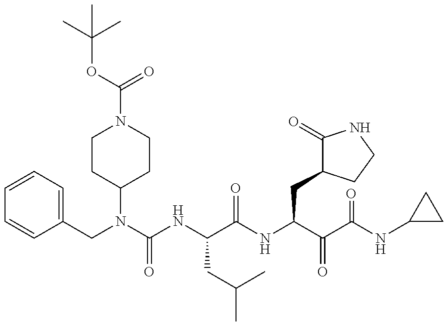 [SMILES: CC(C)C[C@H](NC(=O)N(Cc1ccccc1)C1CCN(C(=O)OC(C)(C)C)CC1)C(=O)N[C@@H](C[C@@H]1CCNC1=O)C(=O)C(=O)NC1CC1] |
| C99 | 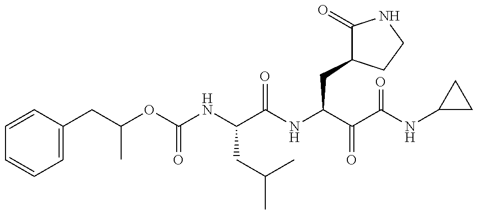 [SMILES: CC(C)C[C@H](NC(=O)OC(C)Cc1ccccc1)C(=O)N[C@@H](C[C@@H]1CCNC1=O)C(=O)C(=O)NC1CC1] |
| C100 | 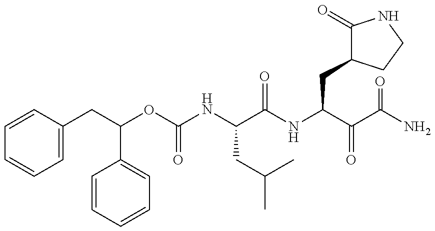 [SMILES: CC(C)C[C@H](NC(=O)OC(Cc1ccccc1)c1ccccc1)C(=O)N[C@@H](C[C@@H]1CCNC1=O)C(=O)C(N)=O] |
| C101 | 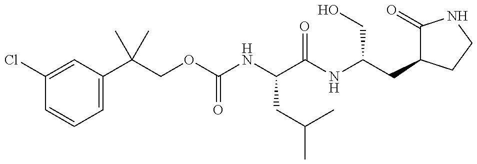 [SMILES: CC(C)C[C@H](NC(=O)OCC(C)(C)c1cccc(Cl)c1)C(=O)N[C@H](CO)C[C@@H]1CCNC1=O] |
| C102 | 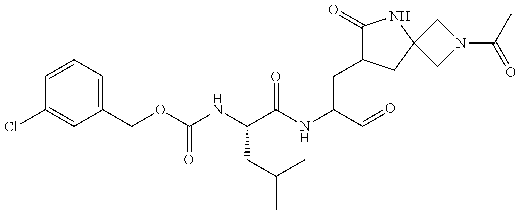 [SMILES: CC(=O)N1CC2(CC(CC(C=O)NC(=O)[C@H](CC(C)C)NC(=O)OCc3cccc(Cl)c3)C(=O)N2)C1] |
| C103 | 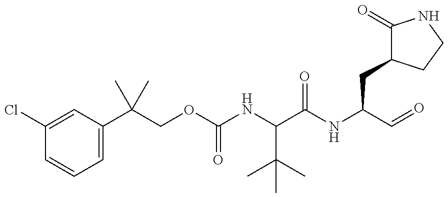 [SMILES: CC(C)(COC(=O)NC(C(=O)N[C@H](C=O)C[C@@H]1CCNC1=O)C(C)(C)C)c1cccc(Cl)c1] |
| C104 | 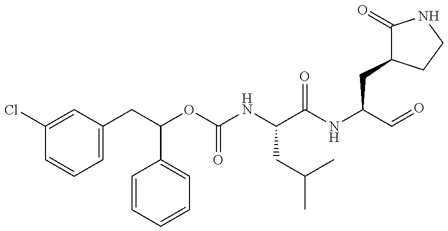 [SMILES: CC(C)C[C@H](NC(=O)OC(Cc1cccc(Cl)c1)c1ccccc1)C(=O)N[C@H](C=O)C[C@@H]1CCNC1=O] |
| C105 | 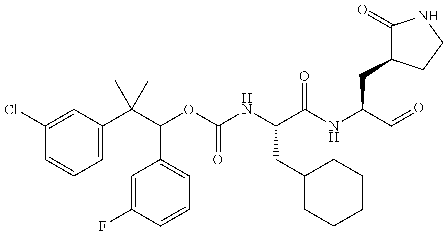 [SMILES: CC(C)(c1cccc(Cl)c1)C(OC(=O)N[C@@H](CC1CCCCC1)C(=O)N[C@H](C=O)C[C@@H]1CCNC1=O)c1cccc(F)c1] |
| C106 | 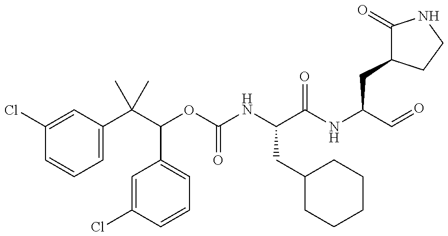 [SMILES: CC(C)(c1cccc(Cl)c1)C(OC(=O)N[C@@H](CC1CCCCC1)C(=O)N[C@H](C=O)C[C@@H]1CCNC1=O)c1cccc(Cl)c1] |
| C107 | 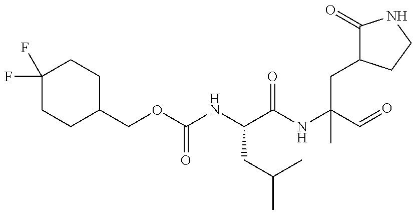 [SMILES: CC(C)C[C@H](NC(=O)OCC1CCC(F)(F)CC1)C(=O)NC(C)(C=O)CC1CCNC1=O] |
| C108 | 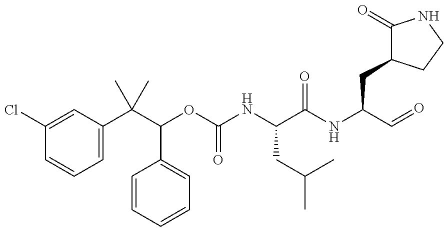 [SMILES: CC(C)C[C@H](NC(=O)OC(c1ccccc1)C(C)(C)c1cccc(Cl)c1)C(=O)N[C@H](C=O)C[C@@H]1CCNC1=O] |
| C109 | 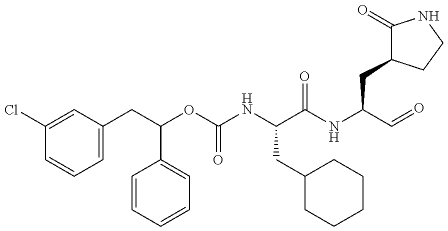 [SMILES: O=C[C@H](C[C@@H]1CCNC1=O)NC(=O)[C@H](CC1CCCCC1)NC(=O)OC(Cc1cccc(Cl)c1)c1ccccc1] |
| C110 | 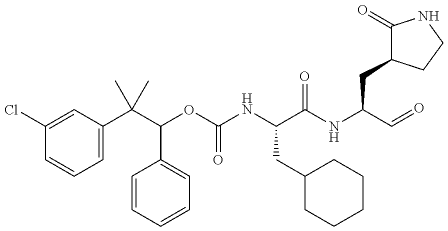 [SMILES: CC(C)(c1cccc(Cl)c1)C(OC(=O)N[C@@H](CC1CCCCC1)C(=O)N[C@H](C=O)C[C@@H]1CCNC1=O)c1ccccc1] |
| C111 | 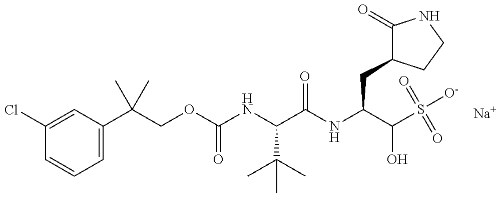 [SMILES: CC(C)(COC(=O)N[C@H](C(=O)N[C@@H](C[C@@H]1CCNC1=O)C(O)S(=O)(=O)[O-])C(C)(C)C)c1cccc(Cl)c1 ; [Na+]] |
| C112 | 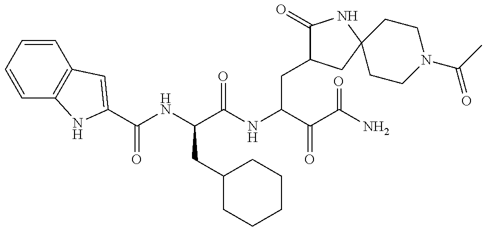 [SMILES: CC(=O)N1CCC2(CC1)CC(CC(NC(=O)[C@@H](CC1CCCCC1)NC(=O)c1cc3ccccc3[nH]1)C(=O)C(N)=O)C(=O)N2] |
| C113 | 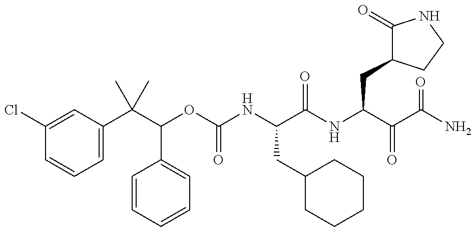 [SMILES: CC(C)(c1cccc(Cl)c1)C(OC(=O)N[C@@H](CC1CCCCC1)C(=O)N[C@@H](C[C@@H]1CCNC1=O)C(=O)C(N)=O)c1ccccc1] |
| C114 | 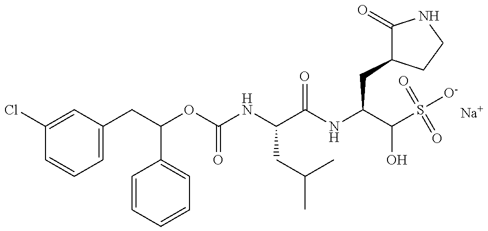 [SMILES: CC(C)C[C@H](NC(=O)OC(Cc1cccc(Cl)c1)c1ccccc1)C(=O)N[C@@H](C[C@@H]1CCNC1=O)C(O)S(=O)(=O)[O-] ; [Na+]] |
| C115 | 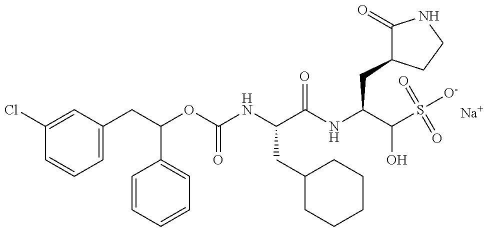 [SMILES: O=C(N[C@@H](CC1CCCCC1)C(=O)N[C@@H](C[C@@H]1CCNC1=O)C(O)S(=O)(=O)[O-])OC(Cc1cccc(Cl)c1)c1ccccc1 ; [Na+]] |
| C116 | 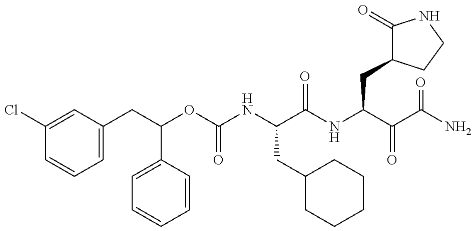 [SMILES: NC(=O)C(=O)[C@H](C[C@@H]1CCNC1=O)NC(=O)[C@H](CC1CCCCC1)NC(=O)OC(Cc1cccc(Cl)c1)c1ccccc1] |
| C117 | 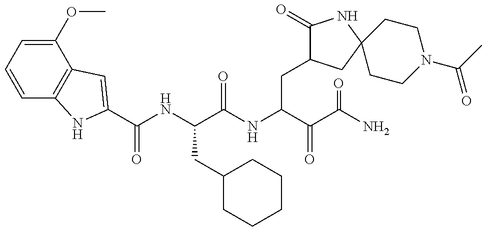 [SMILES: COc1cccc2[nH]c(C(=O)N[C@@H](CC3CCCCC3)C(=O)NC(CC3CC4(CCN(C(C)=O)CC4)NC3=O)C(=O)C(N)=O)cc12] |
| C118 | 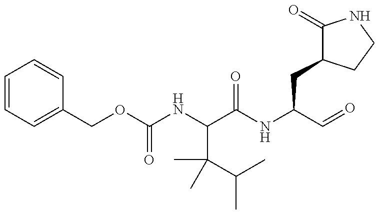 [SMILES: CC(C)C(C)(C)C(NC(=O)OCc1ccccc1)C(=O)N[C@H](C=O)C[C@@H]1CCNC1=O] |
| C119 | 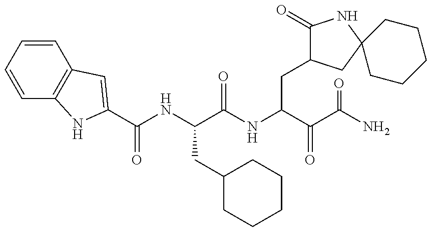 [SMILES: NC(=O)C(=O)C(CC1CC2(CCCCC2)NC1=O)NC(=O)[C@H](CC1CCCCC1)NC(=O)c1cc2ccccc2[nH]1] |
| C120 | 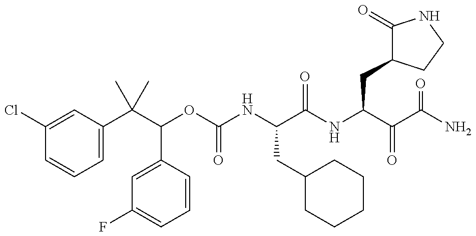 [SMILES: CC(C)(c1cccc(Cl)c1)C(OC(=O)N[C@@H](CC1CCCCC1)C(=O)N[C@@H](C[C@@H]1CCNC1=O)C(=O)C(N)=O)c1cccc(F)c1] |
| C121 | 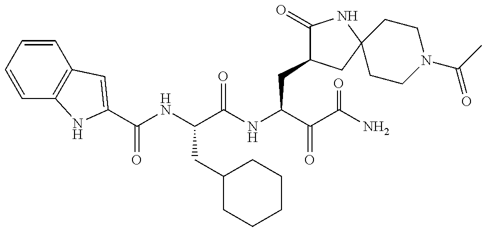 [SMILES: CC(=O)N1CCC2(CC1)C[C@@H](C[C@H](NC(=O)[C@H](CC1CCCCC1)NC(=O)c1cc3ccccc3[nH]1)C(=O)C(N)=O)C(=O)N2] |
| C122 | 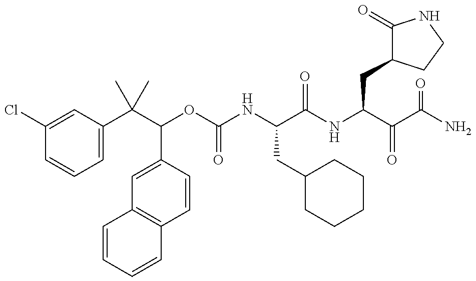 [SMILES: CC(C)(c1cccc(Cl)c1)C(OC(=O)N[C@@H](CC1CCCCC1)C(=O)N[C@@H](C[C@@H]1CCNC1=O)C(=O)C(N)=O)c1ccc2ccccc2c1] |
| C123 | 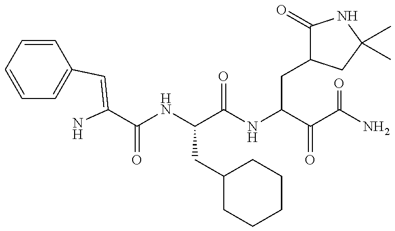 [SMILES: CC1(C)CC(CC(NC(=O)[C@H](CC2CCCCC2)NC(=O)/C(N)=C/c2ccccc2)C(=O)C(N)=O)C(=O)N1] |
| C124 | 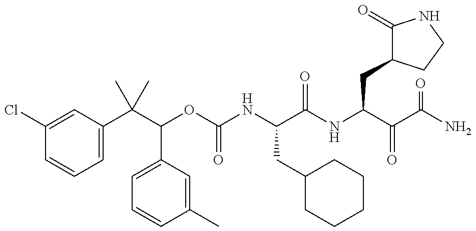 [SMILES: Cc1cccc(C(OC(=O)N[C@@H](CC2CCCCC2)C(=O)N[C@@H](C[C@@H]2CCNC2=O)C(=O)C(N)=O)C(C)(C)c2cccc(Cl)c2)c1] |
| C125 | 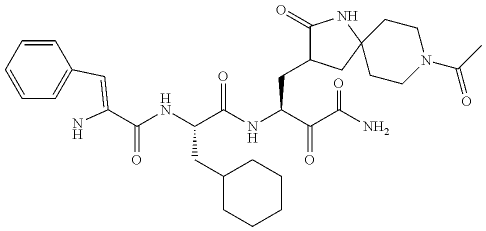 [SMILES: CC(=O)N1CCC2(CC1)CC(C[C@H](NC(=O)[C@H](CC1CCCCC1)NC(=O)/C(N)=C/c1ccccc1)C(=O)C(N)=O)C(=O)N2] |
| C126 | 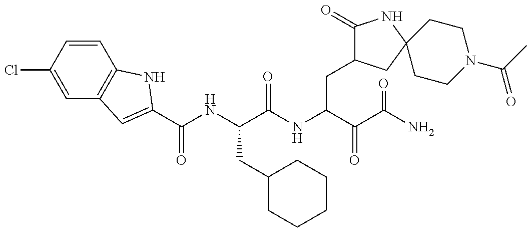 [SMILES: CC(=O)N1CCC2(CC1)CC(CC(NC(=O)[C@H](CC1CCCCC1)NC(=O)c1cc3cc(Cl)ccc3[nH]1)C(=O)C(N)=O)C(=O)N2] |
| C127 | 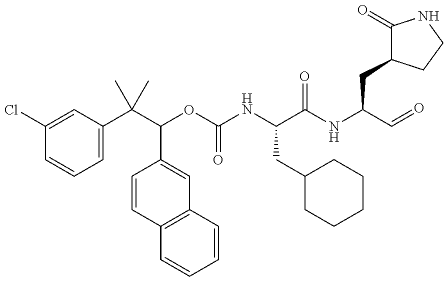 [SMILES: CC(C)(c1cccc(Cl)c1)C(OC(=O)N[C@@H](CC1CCCCC1)C(=O)N[C@H](C=O)C[C@@H]1CCNC1=O)c1ccc2ccccc2c1] |
| C128 | 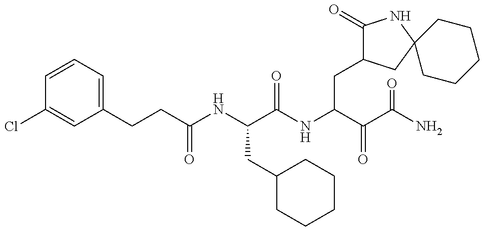 [SMILES: NC(=O)C(=O)C(CC1CC2(CCCCC2)NC1=O)NC(=O)[C@H](CC1CCCCC1)NC(=O)CCc1cccc(Cl)c1] |
| C129 | 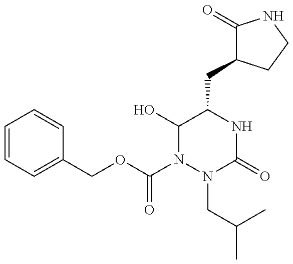 [SMILES: CC(C)CN1C(=O)N[C@@H](C[C@@H]2CCNC2=O)C(O)N1C(=O)OCc1ccccc1] |
| C130 | 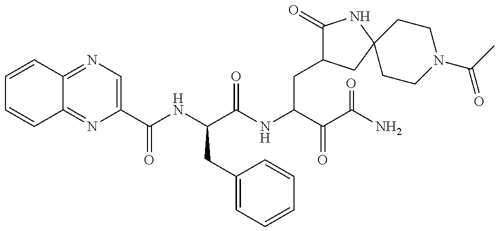 [SMILES: CC(=O)N1CCC2(CC1)CC(CC(NC(=O)[C@@H](Cc1ccccc1)NC(=O)c1cnc3ccccc3n1)C(=O)C(N)=O)C(=O)N2] |
| C131 | 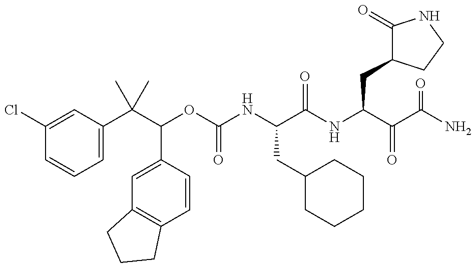 [SMILES: CC(C)(c1cccc(Cl)c1)C(OC(=O)N[C@@H](CC1CCCCC1)C(=O)N[C@@H](C[C@@H]1CCNC1=O)C(=O)C(N)=O)c1ccc2c(c1)CCC2] |
| C132 | 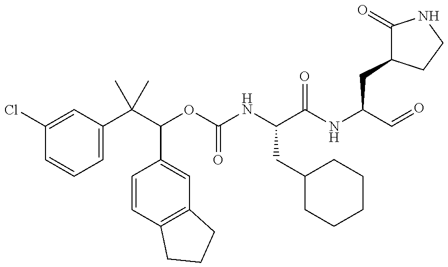 [SMILES: CC(C)(c1cccc(Cl)c1)C(OC(=O)N[C@@H](CC1CCCCC1)C(=O)N[C@H](C=O)C[C@@H]1CCNC1=O)c1ccc2c(c1)CCC2] |
| C133 | 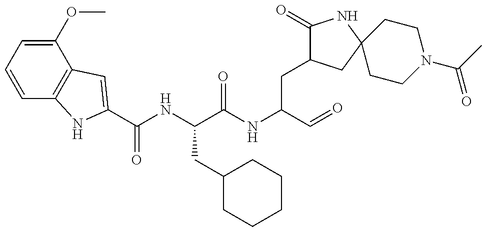 [SMILES: COc1cccc2[nH]c(C(=O)N[C@@H](CC3CCCCC3)C(=O)NC(C=O)CC3CC4(CCN(C(C)=O)CC4)NC3=O)cc12] |
| C134 | 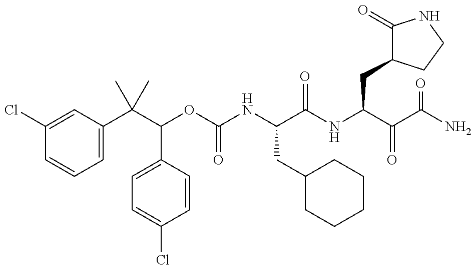 [SMILES: CC(C)(c1cccc(Cl)c1)C(OC(=O)N[C@@H](CC1CCCCC1)C(=O)N[C@@H](C[C@@H]1CCNC1=O)C(=O)C(N)=O)c1ccc(Cl)cc1] |
| C135 | 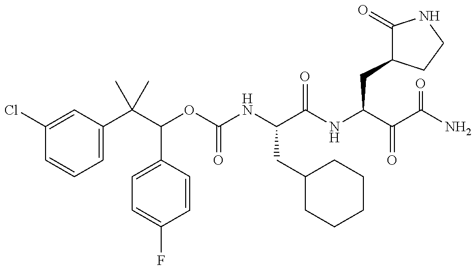 [SMILES: CC(C)(c1cccc(Cl)c1)C(OC(=O)N[C@@H](CC1CCCCC1)C(=O)N[C@@H](C[C@@H]1CCNC1=O)C(=O)C(N)=O)c1ccc(F)cc1] |
| C136 | 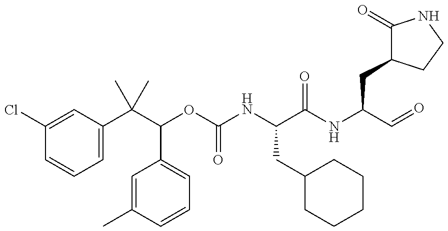 [SMILES: Cc1cccc(C(OC(=O)N[C@@H](CC2CCCCC2)C(=O)N[C@H](C=O)C[C@@H]2CCNC2=O)C(C)(C)c2cccc(Cl)c2)c1] |
| C137 | 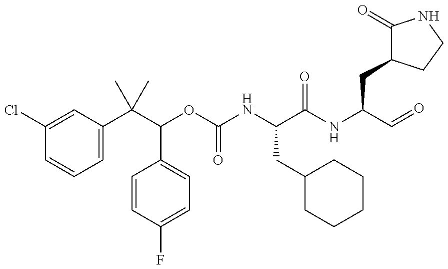 [SMILES: CC(C)(c1cccc(Cl)c1)C(OC(=O)N[C@@H](CC1CCCCC1)C(=O)N[C@H](C=O)C[C@@H]1CCNC1=O)c1ccc(F)cc1] |
| C138 | 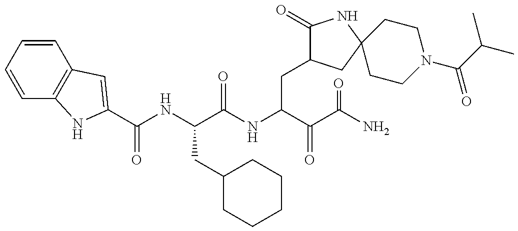 [SMILES: CC(C)C(=O)N1CCC2(CC1)CC(CC(NC(=O)[C@H](CC1CCCCC1)NC(=O)c1cc3ccccc3[nH]1)C(=O)C(N)=O)C(=O)N2] |
| C139 | 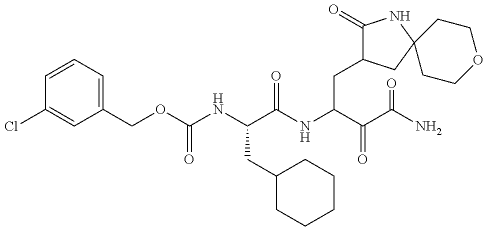 [SMILES: NC(=O)C(=O)C(CC1CC2(CCOCC2)NC1=O)NC(=O)[C@H](CC1CCCCC1)NC(=O)OCc1cccc(Cl)c1] |
| C140 | 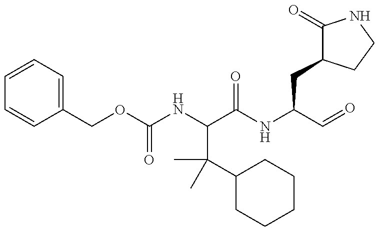 [SMILES: CC(C)(C1CCCCC1)C(NC(=O)OCc1ccccc1)C(=O)N[C@H](C=O)C[C@@H]1CCNC1=O] |
| C141 | 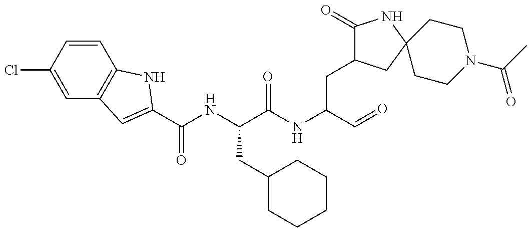 [SMILES: CC(=O)N1CCC2(CC1)CC(CC(C=O)NC(=O)[C@H](CC1CCCCC1)NC(=O)c1cc3cc(Cl)ccc3[nH]1)C(=O)N2] |
| C142 | 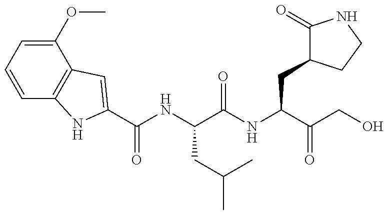 [SMILES: COc1cccc2[nH]c(C(=O)N[C@@H](CC(C)C)C(=O)N[C@@H](C[C@@H]3CCNC3=O)C(=O)CO)cc12] |
| C143 | 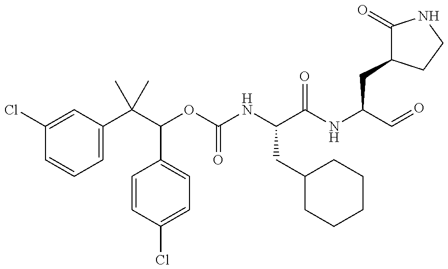 [SMILES: CC(C)(c1cccc(Cl)c1)C(OC(=O)N[C@@H](CC1CCCCC1)C(=O)N[C@H](C=O)C[C@@H]1CCNC1=O)c1ccc(Cl)cc1] |
| C144 | 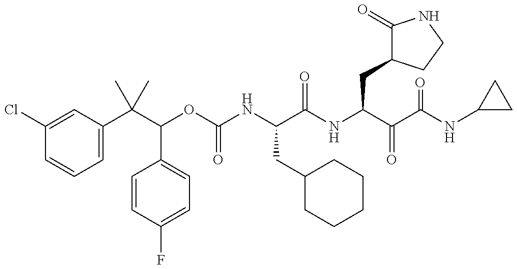 [SMILES: CC(C)(c1cccc(Cl)c1)C(OC(=O)N[C@@H](CC1CCCCC1)C(=O)N[C@@H](C[C@@H]1CCNC1=O)C(=O)C(=O)NC1CC1)c1ccc(F)cc1] |
| C145 | 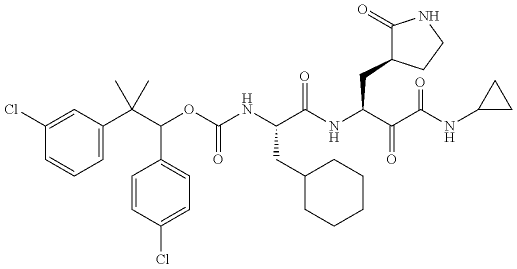 [SMILES: CC(C)(c1cccc(Cl)c1)C(OC(=O)N[C@@H](CC1CCCCC1)C(=O)N[C@@H](C[C@@H]1CCNC1=O)C(=O)C(=O)NC1CC1)c1ccc(Cl)cc1] |
| C146 | 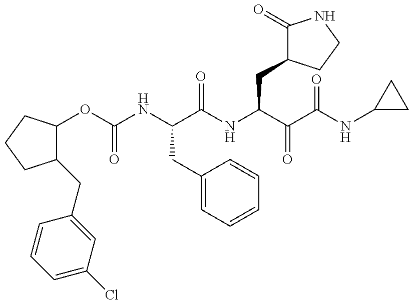 [SMILES: O=C(N[C@@H](Cc1ccccc1)C(=O)N[C@@H](C[C@@H]1CCNC1=O)C(=O)C(=O)NC1CC1)OC1CCCC1Cc1cccc(Cl)c1] |
| C147 | 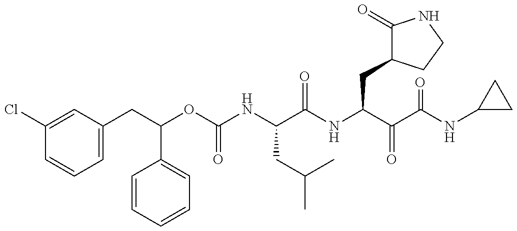 [SMILES: CC(C)C[C@H](NC(=O)OC(Cc1cccc(Cl)c1)c1ccccc1)C(=O)N[C@@H](C[C@@H]1CCNC1=O)C(=O)C(=O)NC1CC1] |
| C148 | 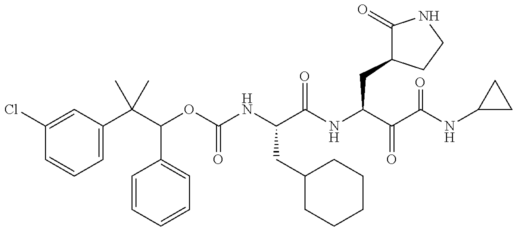 [SMILES: CC(C)(c1cccc(Cl)c1)C(OC(=O)N[C@@H](CC1CCCCC1)C(=O)N[C@@H](C[C@@H]1CCNC1=O)C(=O)C(=O)NC1CC1)c1ccccc1] |
| C149 | 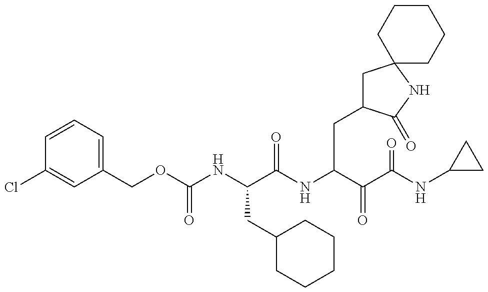 [SMILES: O=C(N[C@@H](CC1CCCCC1)C(=O)NC(CC1CC2(CCCCC2)NC1=O)C(=O)C(=O)NC1CC1)OCc1cccc(Cl)c1] |
| C150 | 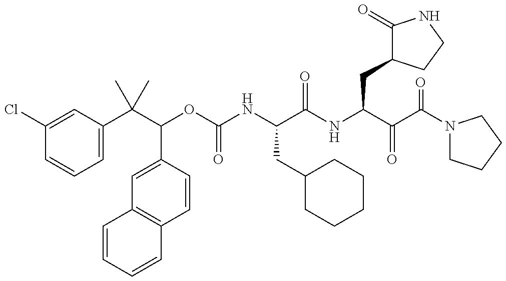 [SMILES: CC(C)(c1cccc(Cl)c1)C(OC(=O)N[C@@H](CC1CCCCC1)C(=O)N[C@@H](C[C@@H]1CCNC1=O)C(=O)C(=O)N1CCCC1)c1ccc2ccccc2c1] |
| C151 | 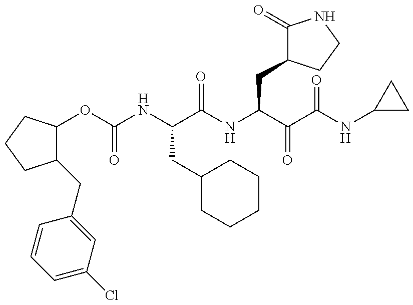 [SMILES: O=C(N[C@@H](CC1CCCCC1)C(=O)N[C@@H](C[C@@H]1CCNC1=O)C(=O)C(=O)NC1CC1)OC1CCCC1Cc1cccc(Cl)c1] |
| C152 | 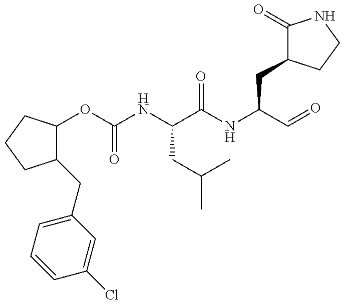 [SMILES: CC(C)C[C@H](NC(=O)OC1CCCC1Cc1cccc(Cl)c1)C(=O)N[C@H](C=O)C[C@@H]1CCNC1=O] |
| C153 | 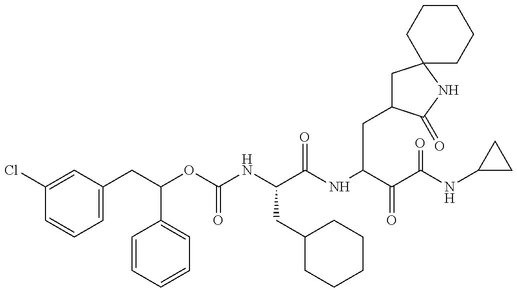 [SMILES: O=C(N[C@@H](CC1CCCCC1)C(=O)NC(CC1CC2(CCCCC2)NC1=O)C(=O)C(=O)NC1CC1)OC(Cc1cccc(Cl)c1)c1ccccc1] |
| C154 | 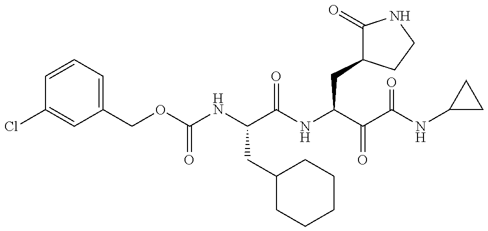 [SMILES: O=C(N[C@@H](CC1CCCCC1)C(=O)N[C@@H](C[C@@H]1CCNC1=O)C(=O)C(=O)NC1CC1)OCc1cccc(Cl)c1] |
| C155 | 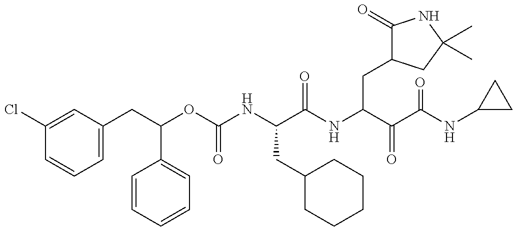 [SMILES: CC1(C)CC(CC(NC(=O)[C@H](CC2CCCCC2)NC(=O)OC(Cc2cccc(Cl)c2)c2ccccc2)C(=O)C(=O)NC2CC2)C(=O)N1] |
| C156 | 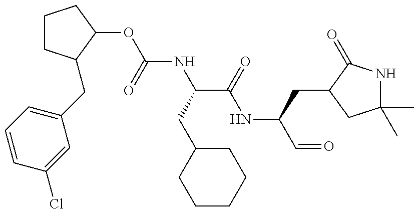 [SMILES: CC1(C)CC(C[C@@H](C=O)NC(=O)[C@H](CC2CCCCC2)NC(=O)OC2CCCC2Cc2cccc(Cl)c2)C(=O)N1] |
| C157 | 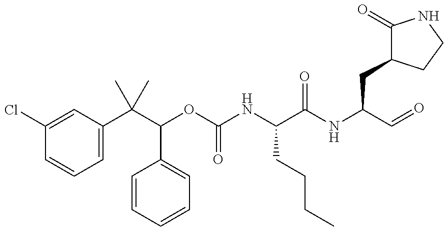 [SMILES: CCCC[C@H](NC(=O)OC(c1ccccc1)C(C)(C)c1cccc(Cl)c1)C(=O)N[C@H](C=O)C[C@@H]1CCNC1=O] |
| C158 | 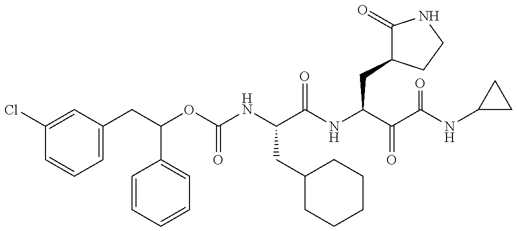 [SMILES: O=C(N[C@@H](CC1CCCCC1)C(=O)N[C@@H](C[C@@H]1CCNC1=O)C(=O)C(=O)NC1CC1)OC(Cc1cccc(Cl)c1)c1ccccc1] |
| C159 | 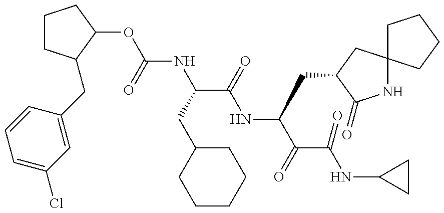 [SMILES: O=C(N[C@@H](CC1CCCCC1)C(=O)N[C@@H](C[C@@H]1CC2(CCCC2)NC1=O)C(=O)C(=O)NC1CC1)OC1CCCC1Cc1cccc(Cl)c1] |
| C160 | 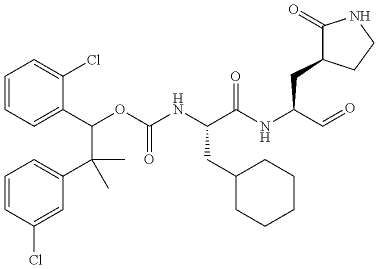 [SMILES: CC(C)(c1cccc(Cl)c1)C(OC(=O)N[C@@H](CC1CCCCC1)C(=O)N[C@H](C=O)C[C@@H]1CCNC1=O)c1ccccc1Cl] |
| C161 | 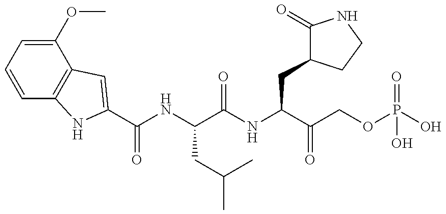 [SMILES: COc1cccc2[nH]c(C(=O)N[C@@H](CC(C)C)C(=O)N[C@@H](C[C@@H]3CCNC3=O)C(=O)COP(=O)(O)O)cc12] |
| C162 | 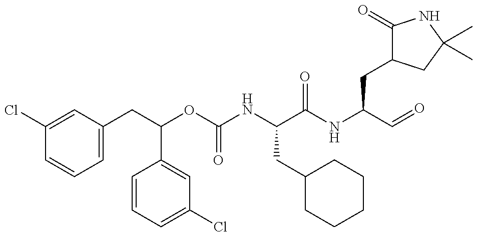 [SMILES: CC1(C)CC(C[C@@H](C=O)NC(=O)[C@H](CC2CCCCC2)NC(=O)OC(Cc2cccc(Cl)c2)c2cccc(Cl)c2)C(=O)N1] |
| C163 | 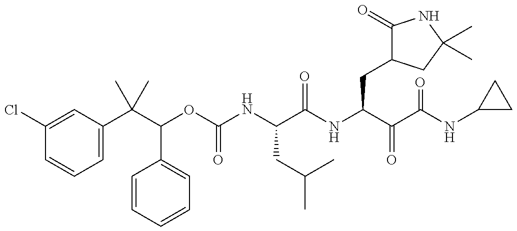 [SMILES: CC(C)C[C@H](NC(=O)OC(c1ccccc1)C(C)(C)c1cccc(Cl)c1)C(=O)N[C@@H](CC1CC(C)(C)NC1=O)C(=O)C(=O)NC1CC1] |
| C164 | 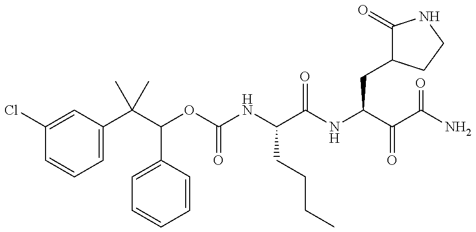 [SMILES: CCCC[C@H](NC(=O)OC(c1ccccc1)C(C)(C)c1cccc(Cl)c1)C(=O)N[C@@H](CC1CCNC1=O)C(=O)C(N)=O] |
| C165 | 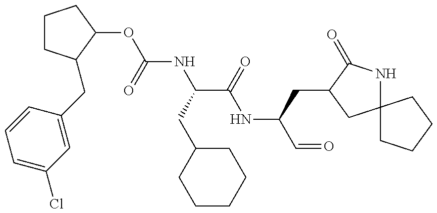 [SMILES: O=C[C@H](CC1CC2(CCCC2)NC1=O)NC(=O)[C@H](CC1CCCCC1)NC(=O)OC1CCCC1Cc1cccc(Cl)c1] |
| C166 | 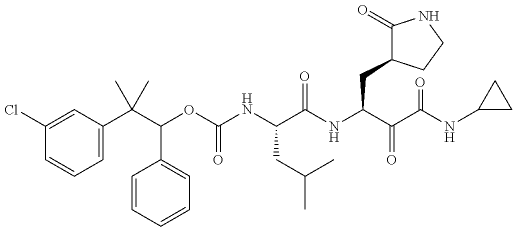 [SMILES: CC(C)C[C@H](NC(=O)OC(c1ccccc1)C(C)(C)c1cccc(Cl)c1)C(=O)N[C@@H](C[C@@H]1CCNC1=O)C(=O)C(=O)NC1CC1] |
| C167 | 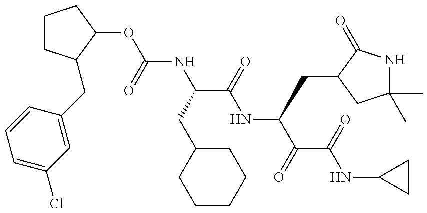 [SMILES: CC1(C)CC(C[C@H](NC(=O)[C@H](CC2CCCCC2)NC(=O)OC2CCCC2Cc2cccc(Cl)c2)C(=O)C(=O)NC2CC2)C(=O)N1] |
| C168 | 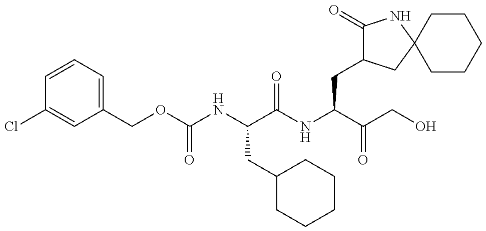 [SMILES: O=C(N[C@@H](CC1CCCCC1)C(=O)N[C@@H](CC1CC2(CCCCC2)NC1=O)C(=O)CO)OCc1cccc(Cl)c1] |
| C169 | 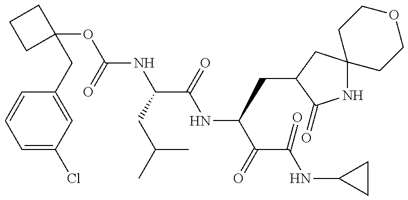 [SMILES: CC(C)C[C@H](NC(=O)OC1(Cc2cccc(Cl)c2)CCC1)C(=O)N[C@@H](CC1CC2(CCOCC2)NC1=O)C(=O)C(=O)NC1CC1] |
| C170 | 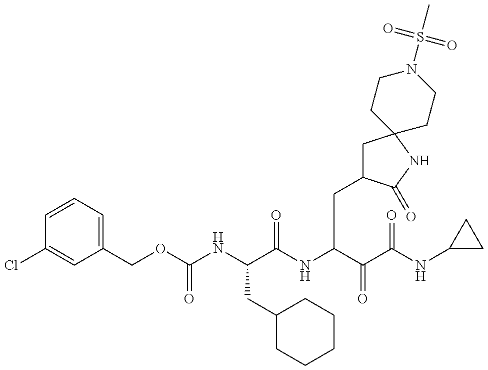 [SMILES: CS(=O)(=O)N1CCC2(CC1)CC(CC(NC(=O)[C@H](CC1CCCCC1)NC(=O)OCc1cccc(Cl)c1)C(=O)C(=O)NC1CC1)C(=O)N2] |
| C171 | 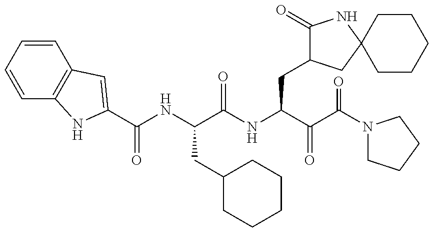 [SMILES: O=C(N[C@@H](CC1CCCCC1)C(=O)N[C@@H](CC1CC2(CCCCC2)NC1=O)C(=O)C(=O)N1CCCC1)c1cc2ccccc2[nH]1] |
| C172 | 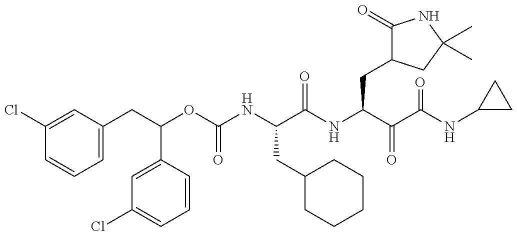 [SMILES: CC1(C)CC(C[C@H](NC(=O)[C@H](CC2CCCCC2)NC(=O)OC(Cc2cccc(Cl)c2)c2cccc(Cl)c2)C(=O)C(=O)NC2CC2)C(=O)N1] |
| C173 | 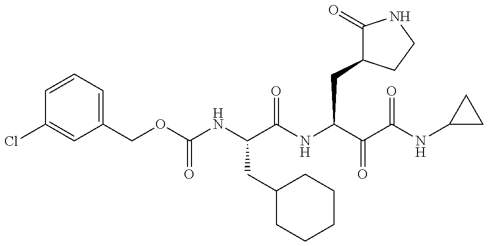 [SMILES: O=C(N[C@@H](CC1CCCCC1)C(=O)N[C@@H](C[C@@H]1CCNC1=O)C(=O)C(=O)NC1CC1)OCc1cccc(Cl)c1] |
| C174 | 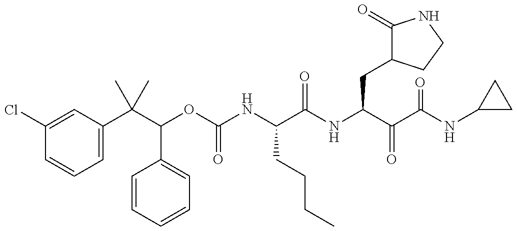 [SMILES: CCCC[C@H](NC(=O)OC(c1ccccc1)C(C)(C)c1cccc(Cl)c1)C(=O)N[C@@H](CC1CCNC1=O)C(=O)C(=O)NC1CC1] |
| C175 | 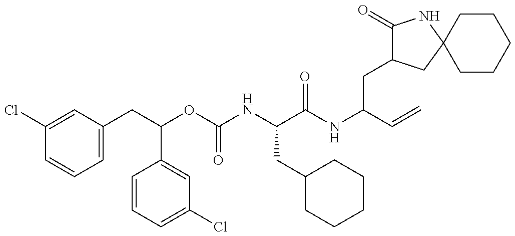 [SMILES: C=CC(CC1CC2(CCCCC2)NC1=O)NC(=O)[C@H](CC1CCCCC1)NC(=O)OC(Cc1cccc(Cl)c1)c1cccc(Cl)c1] |
| C176 | 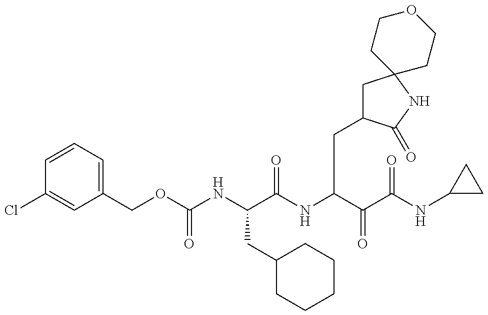 [SMILES: O=C(N[C@@H](CC1CCCCC1)C(=O)NC(CC1CC2(CCOCC2)NC1=O)C(=O)C(=O)NC1CC1)OCc1cccc(Cl)c1] |
| C177 | 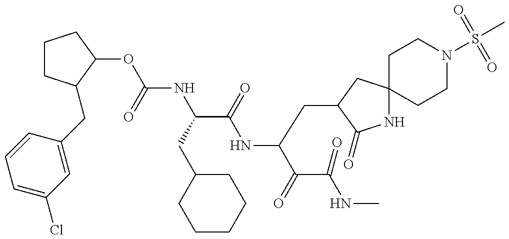 [SMILES: CNC(=O)C(=O)C(CC1CC2(CCN(S(C)(=O)=O)CC2)NC1=O)NC(=O)[C@H](CC1CCCCC1)NC(=O)OC1CCCC1Cc1cccc(Cl)c1] |
| C178 | 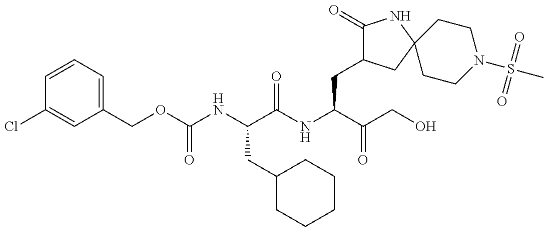 [SMILES: CS(=O)(=O)N1CCC2(CC1)CC(C[C@H](NC(=O)[C@H](CC1CCCCC1)NC(=O)OCc1cccc(Cl)c1)C(=O)CO)C(=O)N2] |
| C179 | 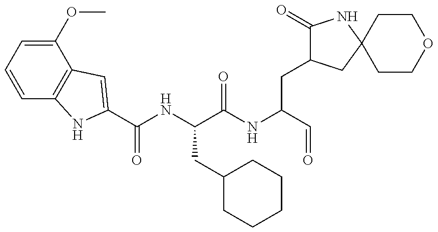 [SMILES: COc1cccc2[nH]c(C(=O)N[C@@H](CC3CCCCC3)C(=O)NC(C=O)CC3CC4(CCOCC4)NC3=O)cc12] |
| C180 | 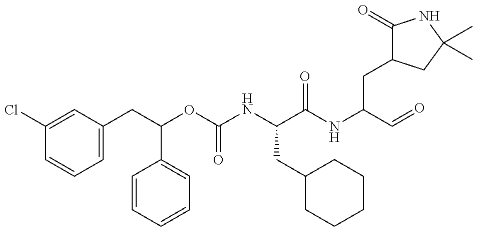 [SMILES: CC1(C)CC(CC(C=O)NC(=O)[C@H](CC2CCCCC2)NC(=O)OC(Cc2cccc(Cl)c2)c2ccccc2)C(=O)N1] |
| C181 | 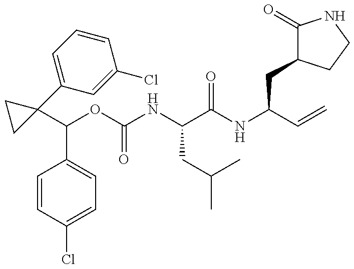 [SMILES: C=C[C@H](C[C@@H]1CCNC1=O)NC(=O)[C@H](CC(C)C)NC(=O)OC(c1ccc(Cl)cc1)C1(c2cccc(Cl)c2)CC1] |
| C182 | 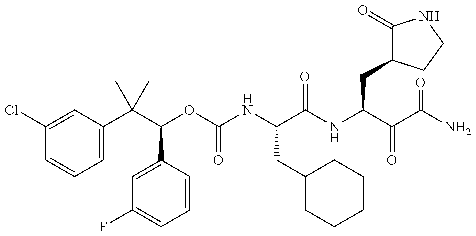 [SMILES: CC(C)(c1cccc(Cl)c1)[C@@H](OC(=O)N[C@@H](CC1CCCCC1)C(=O)N[C@@H](C[C@@H]1CCNC1=O)C(=O)C(N)=O)c1cccc(F)c1] |
| C183 | 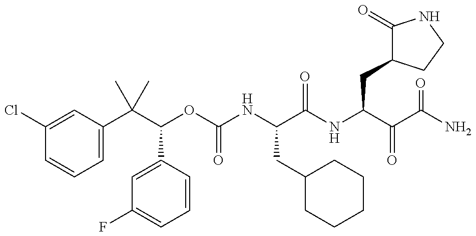 [SMILES: CC(C)(c1cccc(Cl)c1)[C@H](OC(=O)N[C@@H](CC1CCCCC1)C(=O)N[C@@H](C[C@@H]1CCNC1=O)C(=O)C(N)=O)c1cccc(F)c1] |
| C184 | 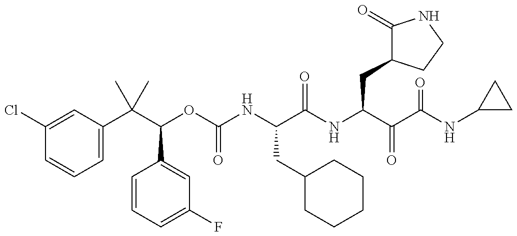 [SMILES: CC(C)(c1cccc(Cl)c1)[C@@H](OC(=O)N[C@@H](CC1CCCCC1)C(=O)N[C@@H](C[C@@H]1CCNC1=O)C(=O)C(=O)NC1CC1)c1cccc(F)c1] |
| C185 | 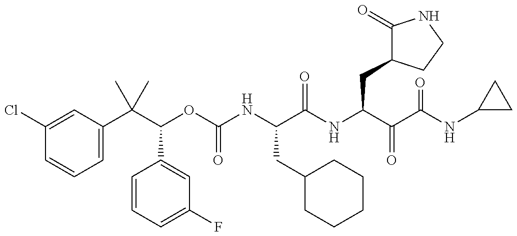 [SMILES: CC(C)(c1cccc(Cl)c1)[C@H](OC(=O)N[C@@H](CC1CCCCC1)C(=O)N[C@@H](C[C@@H]1CCNC1=O)C(=O)C(=O)NC1CC1)c1cccc(F)c1] |
| C186 | 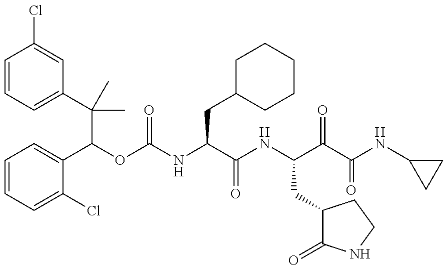 [SMILES: CC(C)(c1cccc(Cl)c1)C(OC(=O)N[C@@H](CC1CCCCC1)C(=O)N[C@@H](C[C@@H]1CCNC1=O)C(=O)C(=O)NC1CC1)c1ccccc1Cl] |
| C187 | 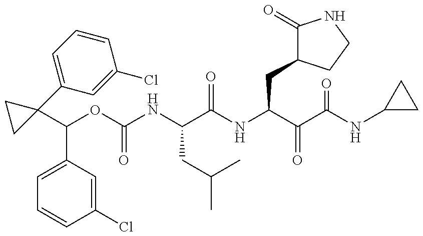 [SMILES: CC(C)C[C@H](NC(=O)OC(c1cccc(Cl)c1)C1(c2cccc(Cl)c2)CC1)C(=O)N[C@@H](C[C@@H]1CCNC1=O)C(=O)C(=O)NC1CC1] |
| C188 | 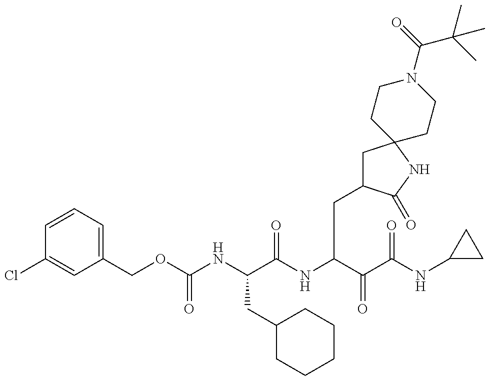 [SMILES: CC(C)(C)C(=O)N1CCC2(CC1)CC(CC(NC(=O)[C@H](CC1CCCCC1)NC(=O)OCc1cccc(Cl)c1)C(=O)C(=O)NC1CC1)C(=O)N2] |
| C189 | 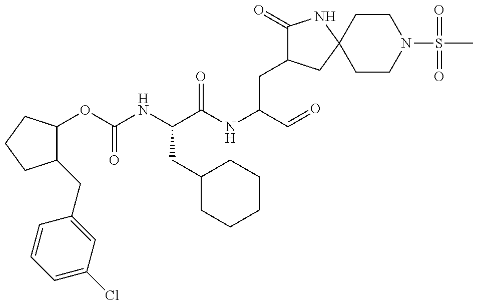 [SMILES: CS(=O)(=O)N1CCC2(CC1)CC(CC(C=O)NC(=O)[C@H](CC1CCCCC1)NC(=O)OC1CCCC1Cc1cccc(Cl)c1)C(=O)N2] |
| C190 | 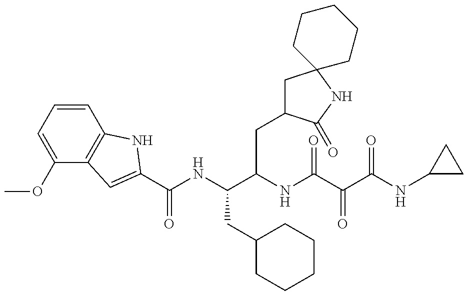 [SMILES: COc1cccc2[nH]c(C(=O)N[C@@H](CC3CCCCC3)C(CC3CC4(CCCCC4)NC3=O)NC(=O)C(=O)C(=O)NC3CC3)cc12] |
| C191 | 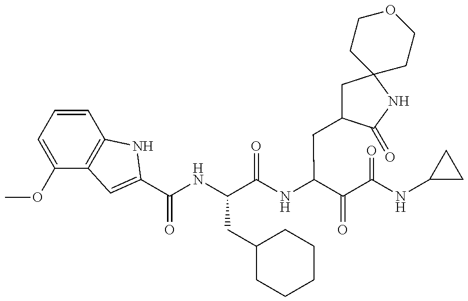 [SMILES: COc1cccc2[nH]c(C(=O)N[C@@H](CC3CCCCC3)C(=O)NC(CC3CC4(CCOCC4)NC3=O)C(=O)C(=O)NC3CC3)cc12] |
| C192 | 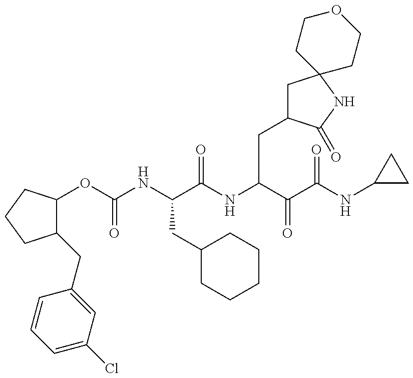 [SMILES: O=C(N[C@@H](CC1CCCCC1)C(=O)NC(CC1CC2(CCOCC2)NC1=O)C(=O)C(=O)NC1CC1)OC1CCCC1Cc1cccc(Cl)c1] |
| C193 | 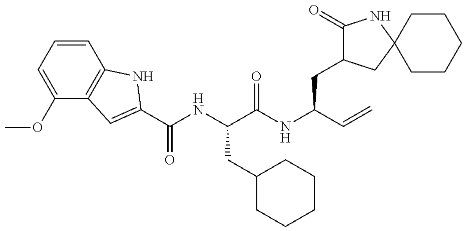 [SMILES: C=C[C@H](CC1CC2(CCCCC2)NC1=O)NC(=O)[C@H](CC1CCCCC1)NC(=O)c1cc2c(OC)cccc2[nH]1] |
| C194 | 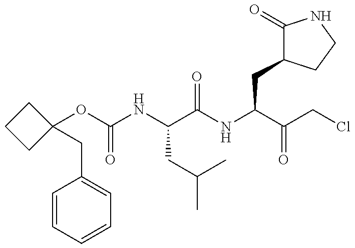 [SMILES: CC(C)C[C@H](NC(=O)OC1(Cc2ccccc2)CCC1)C(=O)N[C@@H](C[C@@H]1CCNC1=O)C(=O)CCl] |
| C195 | 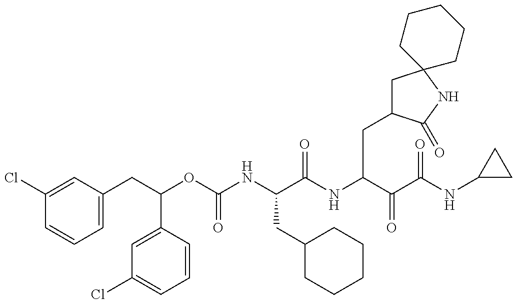 [SMILES: O=C(N[C@@H](CC1CCCCC1)C(=O)NC(CC1CC2(CCCCC2)NC1=O)C(=O)C(=O)NC1CC1)OC(Cc1cccc(Cl)c1)c1cccc(Cl)c1] |
| C196 | 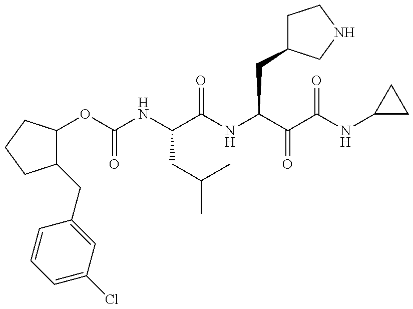 [SMILES: CC(C)C[C@H](NC(=O)OC1CCCC1Cc1cccc(Cl)c1)C(=O)N[C@@H](C[C@H]1CCNC1)C(=O)C(=O)NC1CC1] |
| C197 | 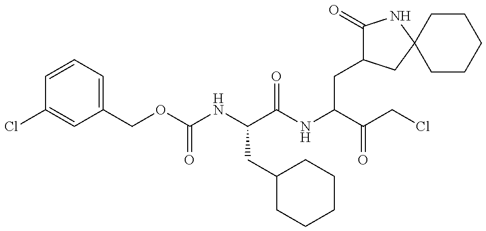 [SMILES: O=C(N[C@@H](CC1CCCCC1)C(=O)NC(CC1CC2(CCCCC2)NC1=O)C(=O)CCl)OCc1cccc(Cl)c1] |
| C198 | 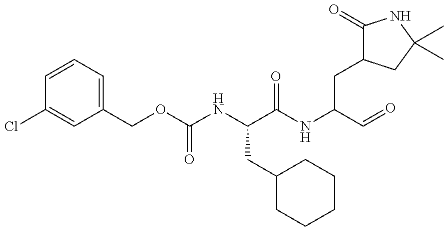 [SMILES: CC1(C)CC(CC(C=O)NC(=O)[C@H](CC2CCCCC2)NC(=O)OCc2cccc(Cl)c2)C(=O)N1] |
| C199 | 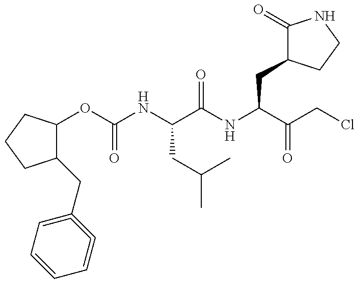 |
| C200 | 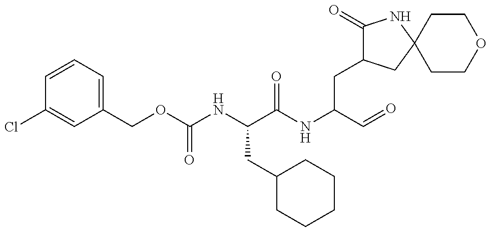 [SMILES: O=CC(CC1CC2(CCOCC2)NC1=O)NC(=O)[C@H](CC1CCCCC1)NC(=O)OCc1cccc(Cl)c1] |
| C201 | 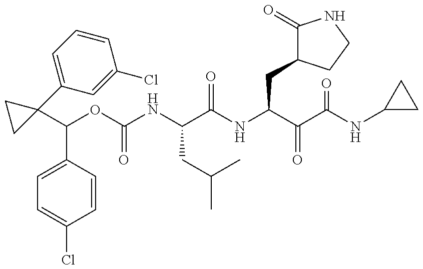 [SMILES: CC(C)C[C@H](NC(=O)OC(c1ccc(Cl)cc1)C1(c2cccc(Cl)c2)CC1)C(=O)N[C@@H](C[C@@H]1CCNC1=O)C(=O)C(=O)NC1CC1] |
| C202 | 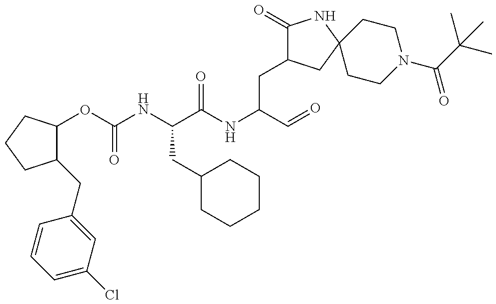 [SMILES: CC(C)(C)C(=O)N1CCC2(CC1)CC(CC(C=O)NC(=O)[C@H](CC1CCCCC1)NC(=O)OC1CCCC1Cc1cccc(Cl)c1)C(=O)N2] |
| C203 | 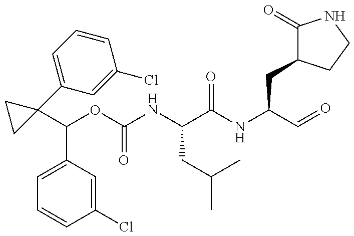 [SMILES: CC(C)C[C@H](NC(=O)OC(c1cccc(Cl)c1)C1(c2cccc(Cl)c2)CC1)C(=O)N[C@H](C=O)C[C@@H]1CCNC1=O] |
| C204 | 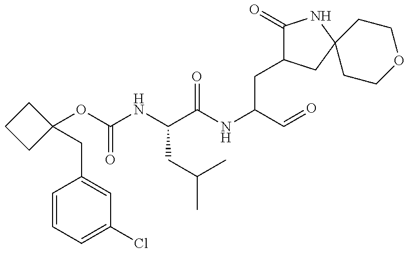 [SMILES: CC(C)C[C@H](NC(=O)OC1(Cc2cccc(Cl)c2)CCC1)C(=O)NC(C=O)CC1CC2(CCOCC2)NC1=O] |
| C205 | 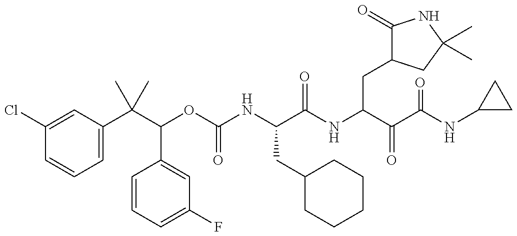 [SMILES: CC1(C)CC(CC(NC(=O)[C@H](CC2CCCCC2)NC(=O)OC(c2cccc(F)c2)C(C)(C)c2cccc(Cl)c2)C(=O)C(=O)NC2CC2)C(=O)N1] |
| C206 | 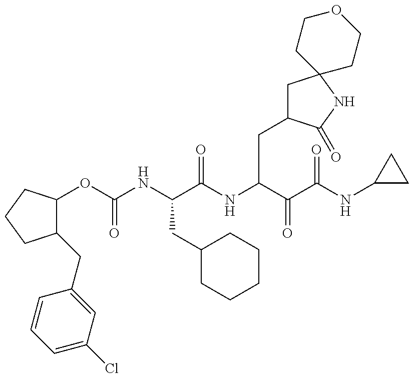 [SMILES: O=C(N[C@@H](CC1CCCCC1)C(=O)NC(CC1CC2(CCOCC2)NC1=O)C(=O)C(=O)NC1CC1)OC1CCCC1Cc1cccc(Cl)c1] |
| C207 | 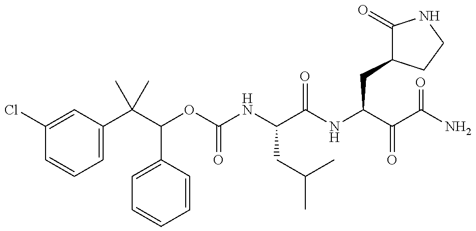 [SMILES: CC(C)C[C@H](NC(=O)OC(c1ccccc1)C(C)(C)c1cccc(Cl)c1)C(=O)N[C@@H](C[C@@H]1CCNC1=O)C(=O)C(N)=O] |
| C208 | 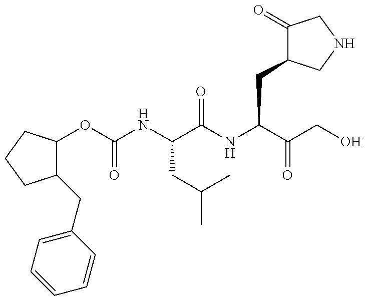 [SMILES: CC(C)C[C@H](NC(=O)OC1CCCC1Cc1ccccc1)C(=O)N[C@@H](C[C@@H]1CNCC1=O)C(=O)CO] |
| C209 | 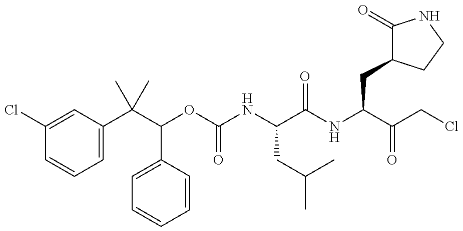 [SMILES: CC(C)C[C@H](NC(=O)OC(c1ccccc1)C(C)(C)c1cccc(Cl)c1)C(=O)N[C@@H](C[C@@H]1CCNC1=O)C(=O)CCl] |
| C210 | 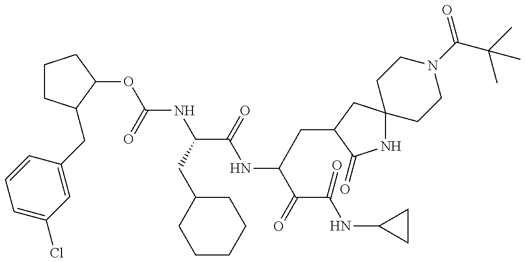 [SMILES: CC(C)(C)C(=O)N1CCC2(CC1)CC(CC(NC(=O)[C@H](CC1CCCCC1)NC(=O)OC1CCCC1Cc1cccc(Cl)c1)C(=O)C(=O)NC1CC1)C(=O)N2] |
| C211 | 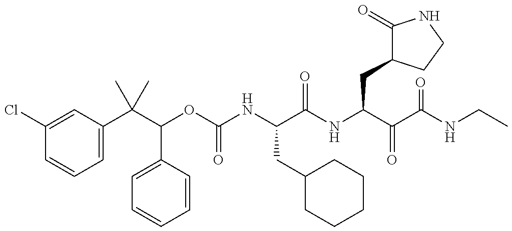 [SMILES: CCNC(=O)C(=O)[C@H](C[C@@H]1CCNC1=O)NC(=O)[C@H](CC1CCCCC1)NC(=O)OC(c1ccccc1)C(C)(C)c1cccc(Cl)c1] |
| C212 | 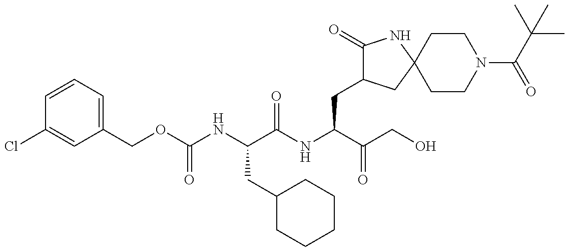 [SMILES: CC(C)(C)C(=O)N1CCC2(CC1)CC(C[C@H](NC(=O)[C@H](CC1CCCCC1)NC(=O)OCc1cccc(Cl)c1)C(=O)CO)C(=O)N2] |
| C213 | 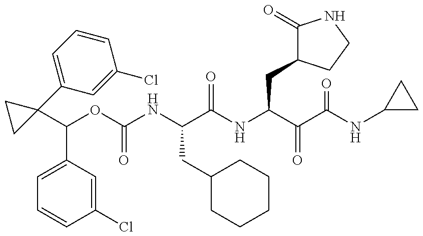 [SMILES: O=C(N[C@@H](CC1CCCCC1)C(=O)N[C@@H](C[C@@H]1CCNC1=O)C(=O)C(=O)NC1CC1)OC(c1cccc(Cl)c1)C1(c2cccc(Cl)c2)CC1] |
| C214 | 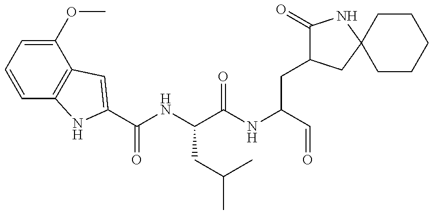 [SMILES: COc1cccc2[nH]c(C(=O)N[C@@H](CC(C)C)C(=O)NC(C=O)CC3CC4(CCCCC4)NC3=O)cc12] |
| C215 | 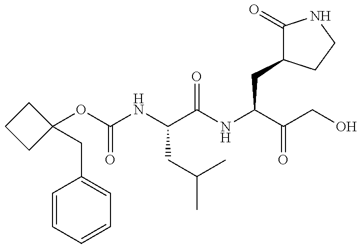 [SMILES: CC(C)C[C@H](NC(=O)OC1(Cc2ccccc2)CCC1)C(=O)N[C@@H](C[C@@H]1CCNC1=O)C(=O)CO] |
| C216 | 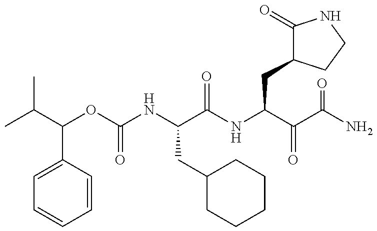 [SMILES: CC(C)C(OC(=O)N[C@@H](CC1CCCCC1)C(=O)N[C@@H](C[C@@H]1CCNC1=O)C(=O)C(N)=O)c1ccccc1] |
| C217 | 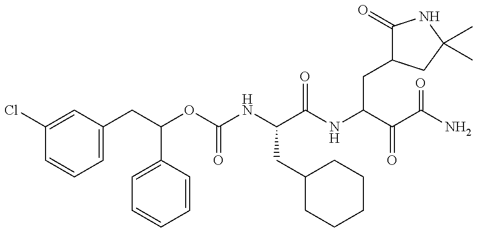 [SMILES: CC1(C)CC(CC(NC(=O)[C@H](CC2CCCCC2)NC(=O)OC(Cc2cccc(Cl)c2)c2ccccc2)C(=O)C(N)=O)C(=O)N1] |
| C218 | 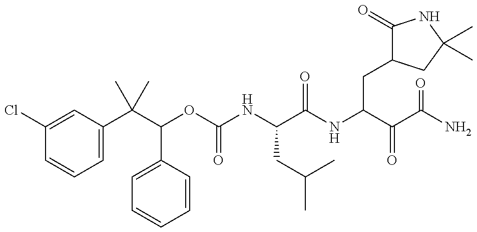 [SMILES: CC(C)C[C@H](NC(=O)OC(c1ccccc1)C(C)(C)c1cccc(Cl)c1)C(=O)NC(CC1CC(C)(C)NC1=O)C(=O)C(N)=O] |
| C219 | 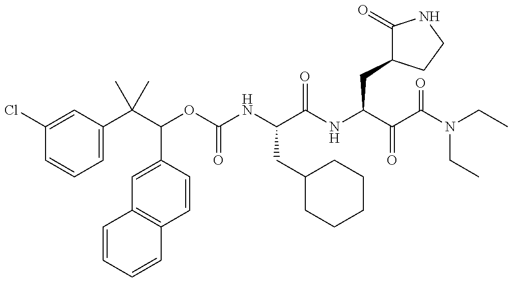 [SMILES: CCN(CC)C(=O)C(=O)[C@H](C[C@@H]1CCNC1=O)NC(=O)[C@H](CC1CCCCC1)NC(=O)OC(c1ccc2ccccc2c1)C(C)(C)c1cccc(Cl)c1] |
| C220 | 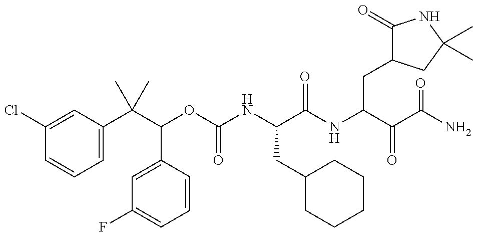 [SMILES: CC1(C)CC(CC(NC(=O)[C@H](CC2CCCCC2)NC(=O)OC(c2cccc(F)c2)C(C)(C)c2cccc(Cl)c2)C(=O)C(N)=O)C(=O)N1] |
| C221 | 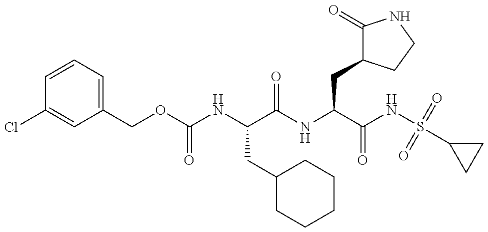 [SMILES: O=C(N[C@@H](CC1CCCCC1)C(=O)N[C@@H](C[C@@H]1CCNC1=O)C(=O)NS(=O)(=O)C1CC1)OCc1cccc(Cl)c1] |
| C222 | 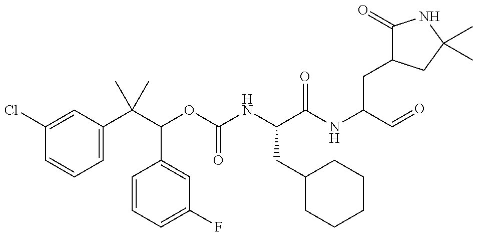 [SMILES: CC1(C)CC(CC(C=O)NC(=O)[C@H](CC2CCCCC2)NC(=O)OC(c2cccc(F)c2)C(C)(C)c2cccc(Cl)c2)C(=O)N1] |
| C223 | 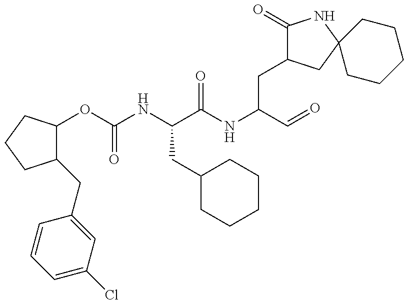 [SMILES: O=CC(CC1CC2(CCCCC2)NC1=O)NC(=O)[C@H](CC1CCCCC1)NC(=O)OC1CCCC1Cc1cccc(Cl)c1] |
| C224 | 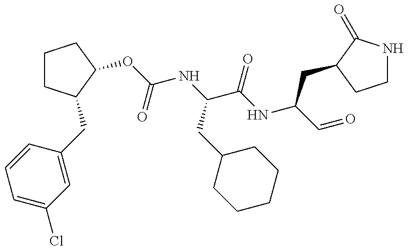 [SMILES: O=C[C@H](C[C@@H]1CCNC1=O)NC(=O)[C@H](CC1CCCCC1)NC(=O)O[C@H]1CCC[C@H]1Cc1cccc(Cl)c1] |
| C225 | 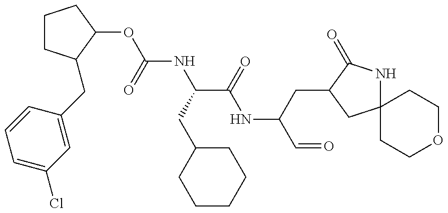 [SMILES: O=CC(CC1CC2(CCOCC2)NC1=O)NC(=O)[C@H](CC1CCCCC1)NC(=O)OC1CCCC1Cc1cccc(Cl)c1] |
| C226 | 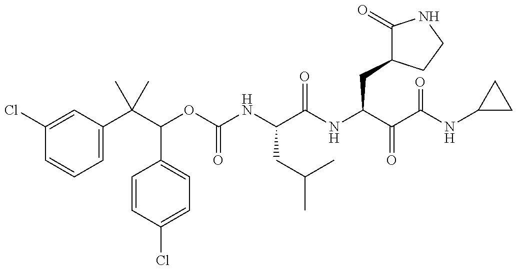 [SMILES: CC(C)C[C@H](NC(=O)OC(c1ccc(Cl)cc1)C(C)(C)c1cccc(Cl)c1)C(=O)N[C@@H](C[C@@H]1CCNC1=O)C(=O)C(=O)NC1CC1] |
| C227 | 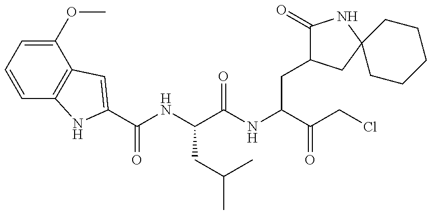 [SMILES: COc1cccc2[nH]c(C(=O)N[C@@H](CC(C)C)C(=O)NC(CC3CC4(CCCCC4)NC3=O)C(=O)CCl)cc12] |
| C228 | 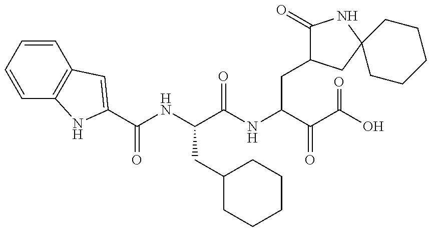 [SMILES: O=C(O)C(=O)C(CC1CC2(CCCCC2)NC1=O)NC(=O)[C@H](CC1CCCCC1)NC(=O)c1cc2ccccc2[nH]1] |
| C229 | 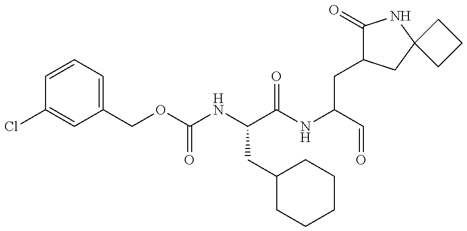 [SMILES: O=CC(CC1CC2(CCC2)NC1=O)NC(=O)[C@H](CC1CCCCC1)NC(=O)OCc1cccc(Cl)c1] |
| C230 | 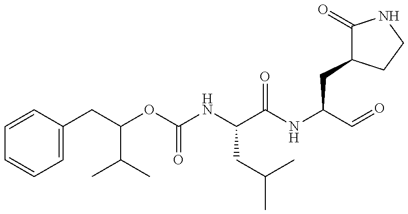 [SMILES: CC(C)C[C@H](NC(=O)OC(Cc1ccccc1)C(C)C)C(=O)N[C@H](C=O)C[C@@H]1CCNC1=O] |
| C231 | 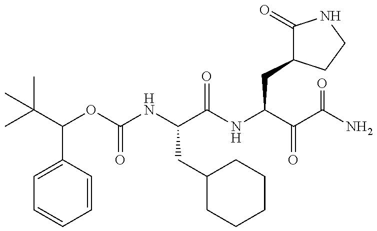 [SMILES: CC(C)(C)C(OC(=O)N[C@@H](CC1CCCCC1)C(=O)N[C@@H](C[C@@H]1CCNC1=O)C(=O)C(N)=O)c1ccccc1] |
| C232 | 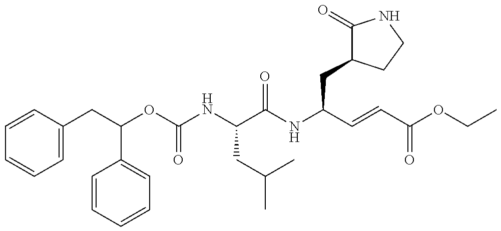 [SMILES: CCOC(=O)/C=C/[C@H](C[C@@H]1CCNC1=O)NC(=O)[C@H](CC(C)C)NC(=O)OC(Cc1ccccc1)c1ccccc1] |
| C233 | 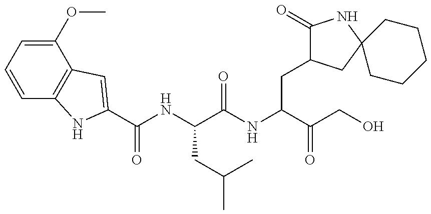 [SMILES: COc1cccc2[nH]c(C(=O)N[C@@H](CC(C)C)C(=O)NC(CC3CC4(CCCCC4)NC3=O)C(=O)CO)cc12] |
| C234 | 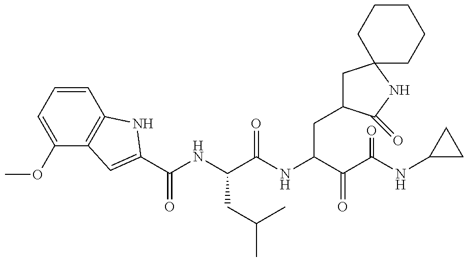 [SMILES: COc1cccc2[nH]c(C(=O)N[C@@H](CC(C)C)C(=O)NC(CC3CC4(CCCCC4)NC3=O)C(=O)C(=O)NC3CC3)cc12] |
| C235 | 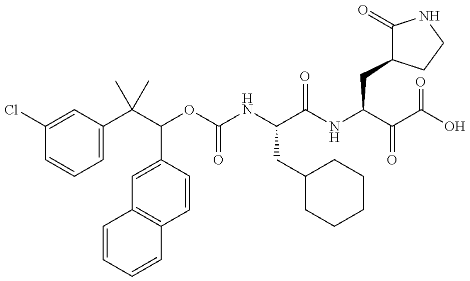 [SMILES: CC(C)(c1cccc(Cl)c1)C(OC(=O)N[C@@H](CC1CCCCC1)C(=O)N[C@@H](C[C@@H]1CCNC1=O)C(=O)C(=O)O)c1ccc2ccccc2c1] |
| C236 | 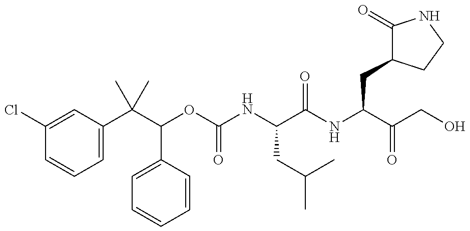 [SMILES: CC(C)C[C@H](NC(=O)OC(c1ccccc1)C(C)(C)c1cccc(Cl)c1)C(=O)N[C@@H](C[C@@H]1CCNC1=O)C(=O)CO] |
| C237 | 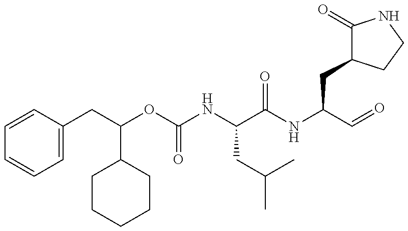 [SMILES: CC(C)C[C@H](NC(=O)OC(Cc1ccccc1)C1CCCCC1)C(=O)N[C@H](C=O)C[C@@H]1CCNC1=O] |
| C238 | 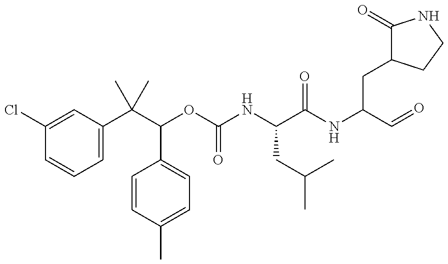 [SMILES: Cc1ccc(C(OC(=O)N[C@@H](CC(C)C)C(=O)NC(C=O)CC2CCNC2=O)C(C)(C)c2cccc(Cl)c2)cc1] |
| C239 | 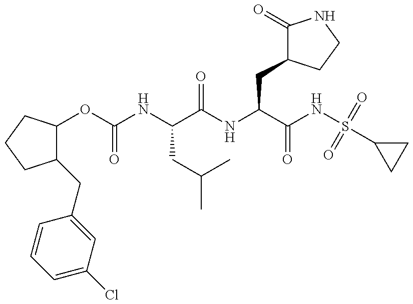 [SMILES: CC(C)C[C@H](NC(=O)OC1CCCC1Cc1cccc(Cl)c1)C(=O)N[C@@H](C[C@@H]1CCNC1=O)C(=O)NS(=O)(=O)C1CC1] |
| C240 | 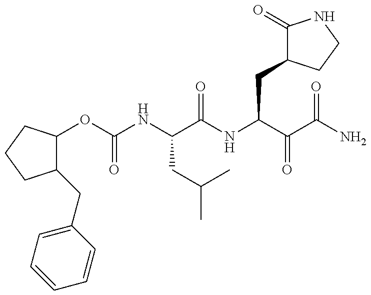 [SMILES: CC(C)C[C@H](NC(=O)OC1CCCC1Cc1ccccc1)C(=O)N[C@@H](C[C@@H]1CCNC1=O)C(=O)C(N)=O] |
| C241 | 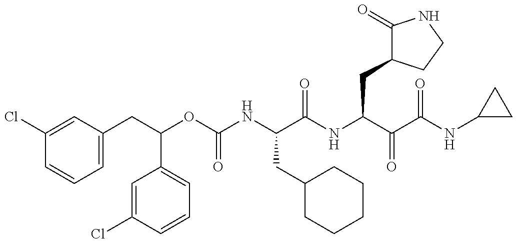 [SMILES: O=C(N[C@@H](CC1CCCCC1)C(=O)N[C@@H](C[C@@H]1CCNC1=O)C(=O)C(=O)NC1CC1)OC(Cc1cccc(Cl)c1)c1cccc(Cl)c1] |
| C242 | 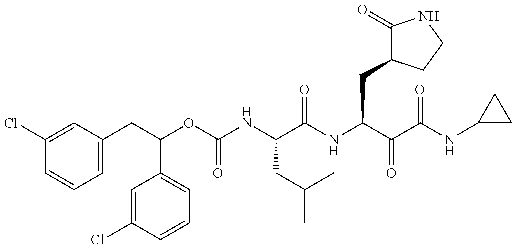 [SMILES: CC(C)C[C@H](NC(=O)OC(Cc1cccc(Cl)c1)c1cccc(Cl)c1)C(=O)N[C@@H](C[C@@H]1CCNC1=O)C(=O)C(=O)NC1CC1] |
| C243 | 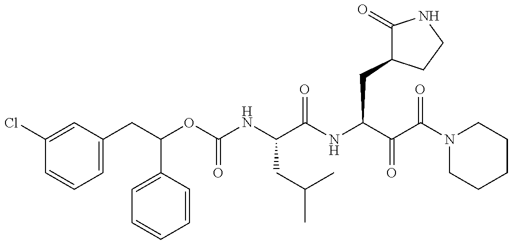 [SMILES: CC(C)C[C@H](NC(=O)OC(Cc1cccc(Cl)c1)c1ccccc1)C(=O)N[C@@H](C[C@@H]1CCNC1=O)C(=O)C(=O)N1CCCCC1] |
| C244 | 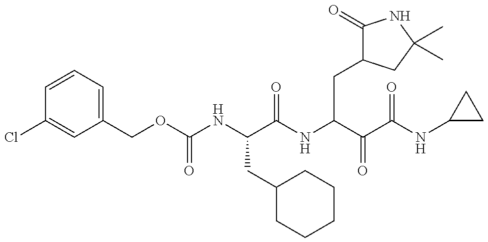 [SMILES: CC1(C)CC(CC(NC(=O)[C@H](CC2CCCCC2)NC(=O)OCc2cccc(Cl)c2)C(=O)C(=O)NC2CC2)C(=O)N1] |
| C245 | 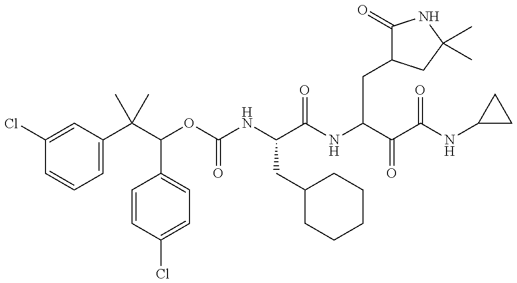 [SMILES: CC1(C)CC(CC(NC(=O)[C@H](CC2CCCCC2)NC(=O)OC(c2ccc(Cl)cc2)C(C)(C)c2cccc(Cl)c2)C(=O)C(=O)NC2CC2)C(=O)N1] |
| C246 | 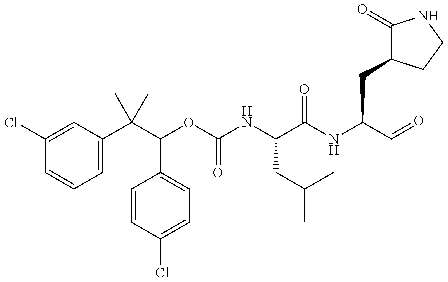 [SMILES: CC(C)C[C@H](NC(=O)OC(c1ccc(Cl)cc1)C(C)(C)c1cccc(Cl)c1)C(=O)N[C@H](C=O)C[C@@H]1CCNC1=O] |
| C247 | 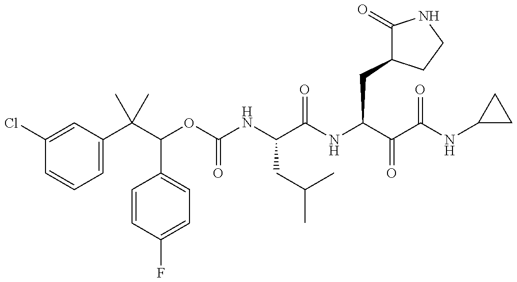 [SMILES: CC(C)C[C@H](NC(=O)OC(c1ccc(F)cc1)C(C)(C)c1cccc(Cl)c1)C(=O)N[C@@H](C[C@@H]1CCNC1=O)C(=O)C(=O)NC1CC1] |
| C248 | 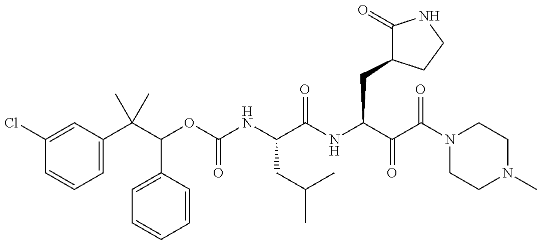 [SMILES: CC(C)C[C@H](NC(=O)OC(c1ccccc1)C(C)(C)c1cccc(Cl)c1)C(=O)N[C@@H](C[C@@H]1CCNC1=O)C(=O)C(=O)N1CCN(C)CC1] |
| C249 | 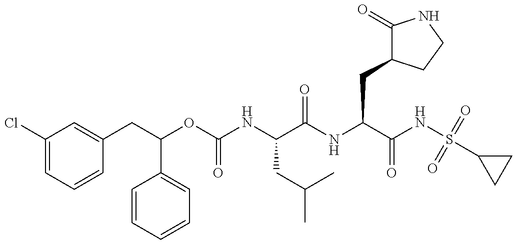 [SMILES: CC(C)C[C@H](NC(=O)OC(Cc1cccc(Cl)c1)c1ccccc1)C(=O)N[C@@H](C[C@@H]1CCNC1=O)C(=O)NS(=O)(=O)C1CC1] |
| C250 | 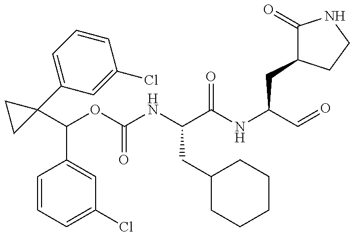 [SMILES: O=C[C@H](C[C@@H]1CCNC1=O)NC(=O)[C@H](CC1CCCCC1)NC(=O)OC(c1cccc(Cl)c1)C1(c2cccc(Cl)c2)CC1] |
| C251 | 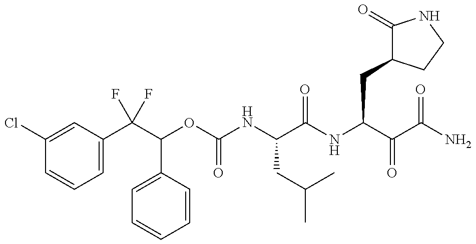 [SMILES: CC(C)C[C@H](NC(=O)OC(c1ccccc1)C(F)(F)c1cccc(Cl)c1)C(=O)N[C@@H](C[C@@H]1CCNC1=O)C(=O)C(N)=O] |
| C252 | 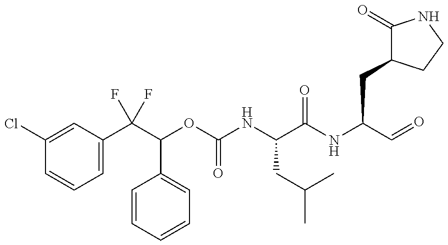 [SMILES: CC(C)C[C@H](NC(=O)OC(c1ccccc1)C(F)(F)c1cccc(Cl)c1)C(=O)N[C@H](C=O)C[C@@H]1CCNC1=O] |
| C253 | 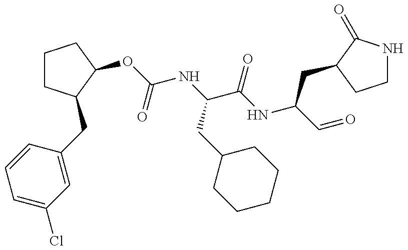 [SMILES: O=C[C@H](C[C@@H]1CCNC1=O)NC(=O)[C@H](CC1CCCCC1)NC(=O)O[C@@H]1CCC[C@@H]1Cc1cccc(Cl)c1] |
| C254 | 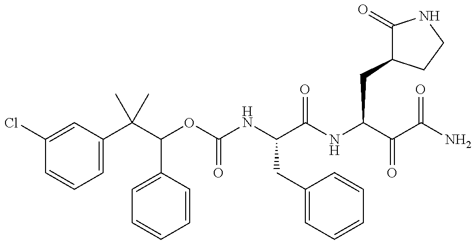 [SMILES: CC(C)(c1cccc(Cl)c1)C(OC(=O)N[C@@H](Cc1ccccc1)C(=O)N[C@@H](C[C@@H]1CCNC1=O)C(=O)C(N)=O)c1ccccc1] |
| C255 | 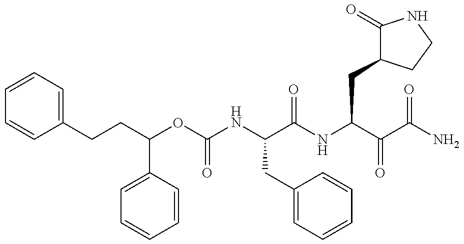 [SMILES: NC(=O)C(=O)[C@H](C[C@@H]1CCNC1=O)NC(=O)[C@H](Cc1ccccc1)NC(=O)OC(CCc1ccccc1)c1ccccc1] |
| C256 | 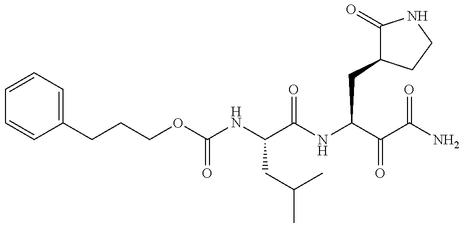 [SMILES: CC(C)C[C@H](NC(=O)OCCCc1ccccc1)C(=O)N[C@@H](C[C@@H]1CCNC1=O)C(=O)C(N)=O] |
| C257 | 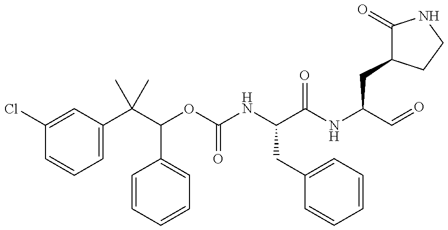 [SMILES: CC(C)(c1cccc(Cl)c1)C(OC(=O)N[C@@H](Cc1ccccc1)C(=O)N[C@H](C=O)C[C@@H]1CCNC1=O)c1ccccc1] |
| C258 | 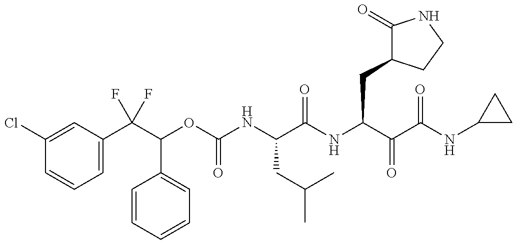 [SMILES: CC(C)C[C@H](NC(=O)OC(c1ccccc1)C(F)(F)c1cccc(Cl)c1)C(=O)N[C@@H](C[C@@H]1CCNC1=O)C(=O)C(=O)NC1CC1] |
| C259 | 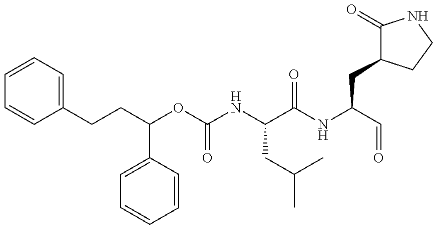 [SMILES: CC(C)C[C@H](NC(=O)OC(CCc1ccccc1)c1ccccc1)C(=O)N[C@H](C=O)C[C@@H]1CCNC1=O] |
| C260 | 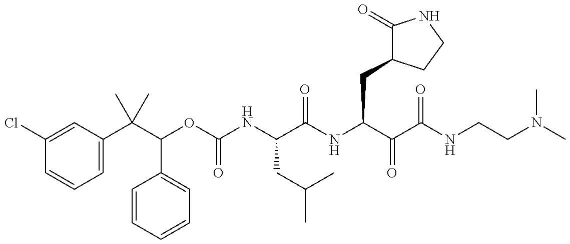 [SMILES: CC(C)C[C@H](NC(=O)OC(c1ccccc1)C(C)(C)c1cccc(Cl)c1)C(=O)N[C@@H](C[C@@H]1CCNC1=O)C(=O)C(=O)NCCN(C)C] |
| C261 | 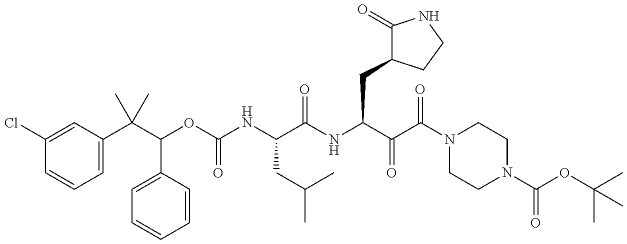 [SMILES: CC(C)C[C@H](NC(=O)OC(c1ccccc1)C(C)(C)c1cccc(Cl)c1)C(=O)N[C@@H](C[C@@H]1CCNC1=O)C(=O)C(=O)N1CCN(C(=O)OC(C)(C)C)CC1] |
| C262 | 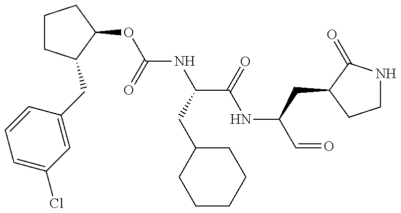 [SMILES: O=C[C@H](C[C@@H]1CCNC1=O)NC(=O)[C@H](CC1CCCCC1)NC(=O)O[C@@H]1CCC[C@H]1Cc1cccc(Cl)c1] |
| C263 | 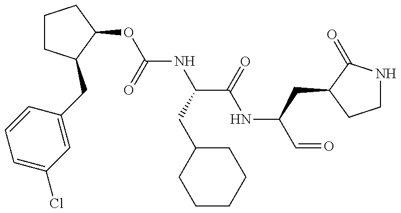 [SMILES: O=C[C@H](C[C@@H]1CCNC1=O)NC(=O)[C@H](CC1CCCCC1)NC(=O)O[C@@H]1CCC[C@@H]1Cc1cccc(Cl)c1] |
| C264 | 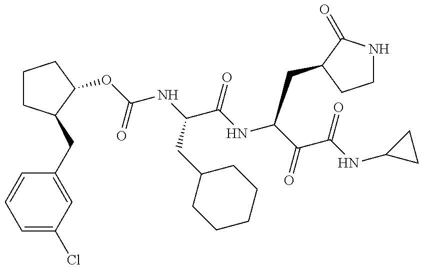 [SMILES: O=C(N[C@@H](CC1CCCCC1)C(=O)N[C@@H](C[C@@H]1CCNC1=O)C(=O)C(=O)NC1CC1)O[C@H]1CCC[C@@H]1Cc1cccc(Cl)c1] |
| C265 | 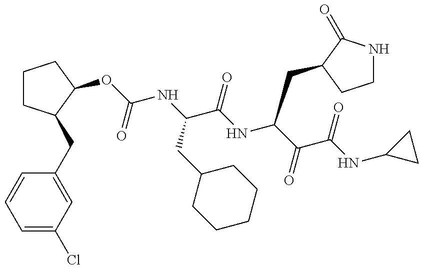 [SMILES: O=C(N[C@@H](CC1CCCCC1)C(=O)N[C@@H](C[C@@H]1CCNC1=O)C(=O)C(=O)NC1CC1)O[C@@H]1CCC[C@@H]1Cc1cccc(Cl)c1] |
| C266 | 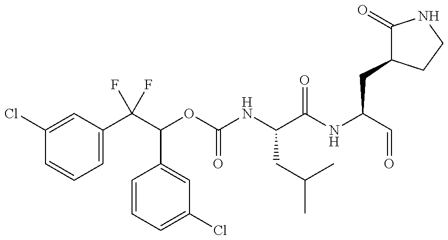 [SMILES: CC(C)C[C@H](NC(=O)OC(c1cccc(Cl)c1)C(F)(F)c1cccc(Cl)c1)C(=O)N[C@H](C=O)C[C@@H]1CCNC1=O] |
| C267 | 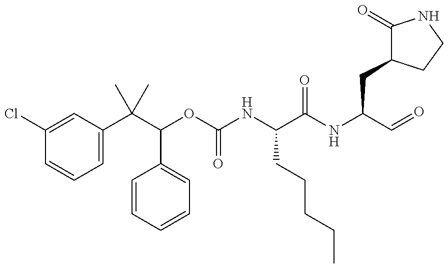 [SMILES: CCCCC[C@H](NC(=O)OC(c1ccccc1)C(C)(C)c1cccc(Cl)c1)C(=O)N[C@H](C=O)C[C@@H]1CCNC1=O] |
| C268 | 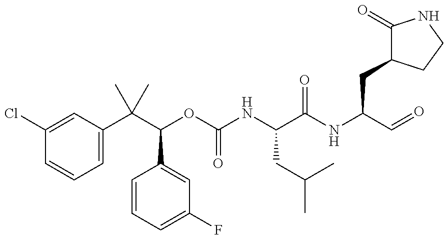 [SMILES: CC(C)C[C@H](NC(=O)O[C@@H](c1cccc(F)c1)C(C)(C)c1cccc(Cl)c1)C(=O)N[C@H](C=O)C[C@@H]1CCNC1=O] |
| C269 | 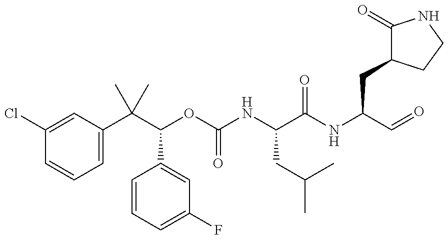 [SMILES: CC(C)C[C@H](NC(=O)O[C@H](c1cccc(F)c1)C(C)(C)c1cccc(Cl)c1)C(=O)N[C@H](C=O)C[C@@H]1CCNC1=O] |
| C270 | 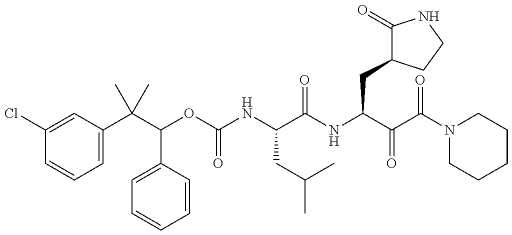 [SMILES: CC(C)C[C@H](NC(=O)OC(c1ccccc1)C(C)(C)c1cccc(Cl)c1)C(=O)N[C@@H](C[C@@H]1CCNC1=O)C(=O)C(=O)N1CCCCC1] |
| C271 | 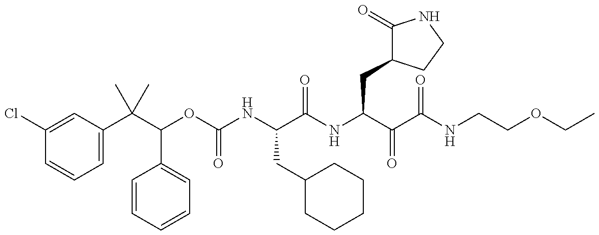 [SMILES: CCOCCNC(=O)C(=O)[C@H](C[C@@H]1CCNC1=O)NC(=O)[C@H](CC1CCCCC1)NC(=O)OC(c1ccccc1)C(C)(C)c1cccc(Cl)c1] |
| C272 | 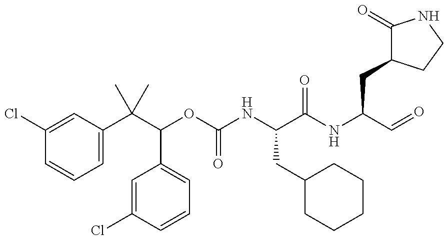 [SMILES: CC(C)(c1cccc(Cl)c1)C(OC(=O)N[C@@H](CC1CCCCC1)C(=O)N[C@H](C=O)C[C@@H]1CCNC1=O)c1cccc(Cl)c1] |
| C273 | 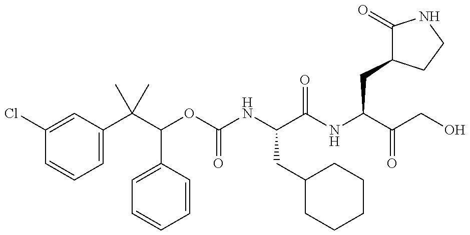 [SMILES: CC(C)(c1cccc(Cl)c1)C(OC(=O)N[C@@H](CC1CCCCC1)C(=O)N[C@@H](C[C@@H]1CCNC1=O)C(=O)CO)c1ccccc1] |
| C274 | 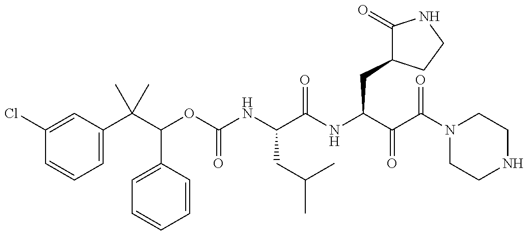 [SMILES: CC(C)C[C@H](NC(=O)OC(c1ccccc1)C(C)(C)c1cccc(Cl)c1)C(=O)N[C@@H](C[C@@H]1CCNC1=O)C(=O)C(=O)N1CCNCC1] |
| C275 | 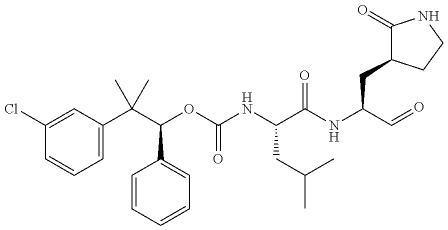 [SMILES: CC(C)C[C@H](NC(=O)O[C@@H](c1ccccc1)C(C)(C)c1cccc(Cl)c1)C(=O)N[C@H](C=O)C[C@@H]1CCNC1=O] |
| C276 | 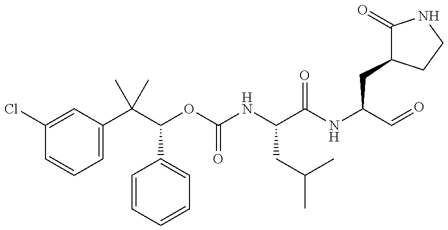 [SMILES: CC(C)C[C@H](NC(=O)O[C@H](c1ccccc1)C(C)(C)c1cccc(Cl)c1)C(=O)N[C@H](C=O)C[C@@H]1CCNC1=O] |
| C277 | 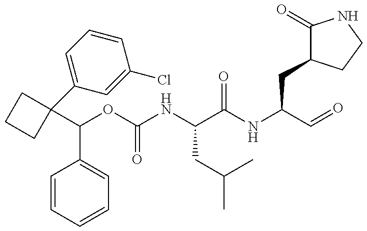 [SMILES: CC(C)C[C@H](NC(=O)OC(c1ccccc1)C1(c2cccc(Cl)c2)CCC1)C(=O)N[C@H](C=O)C[C@@H]1CCNC1=O] |
| C278 | 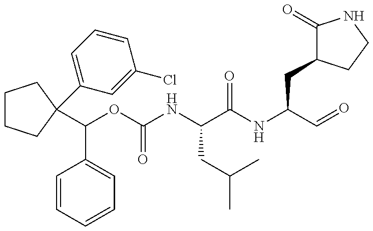 [SMILES: CC(C)C[C@H](NC(=O)OC(c1ccccc1)C1(c2cccc(Cl)c2)CCCC1)C(=O)N[C@H](C=O)C[C@@H]1CCNC1=O] |
| C279 | 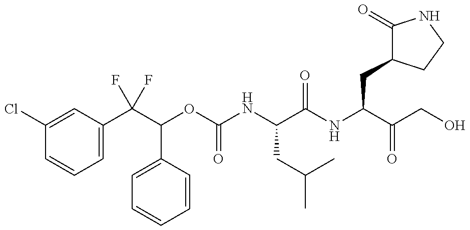 [SMILES: CC(C)C[C@H](NC(=O)OC(c1ccccc1)C(F)(F)c1cccc(Cl)c1)C(=O)N[C@@H](C[C@@H]1CCNC1=O)C(=O)CO] |
| C280 | 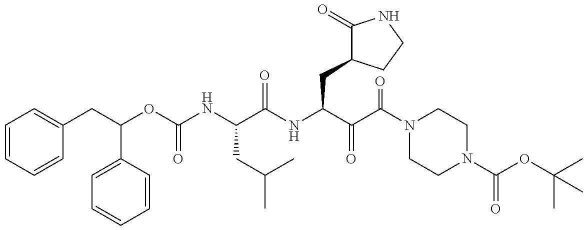 [SMILES: CC(C)C[C@H](NC(=O)OC(Cc1ccccc1)c1ccccc1)C(=O)N[C@@H](C[C@@H]1CCNC1=O)C(=O)C(=O)N1CCN(C(=O)OC(C)(C)C)CC1] |
| C281 | 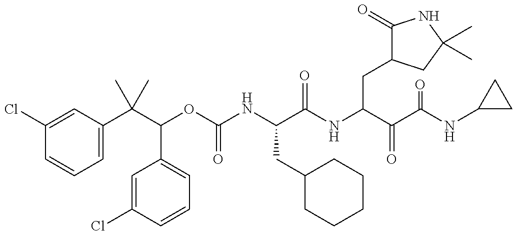 [SMILES: CC1(C)CC(CC(NC(=O)[C@H](CC2CCCCC2)NC(=O)OC(c2cccc(Cl)c2)C(C)(C)c2cccc(Cl)c2)C(=O)C(=O)NC2CC2)C(=O)N1] |
| C282 | 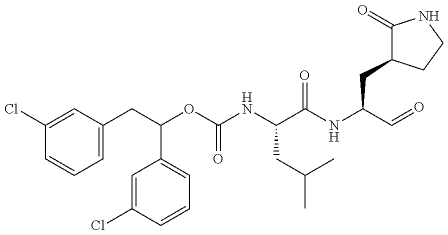 [SMILES: CC(C)C[C@H](NC(=O)OC(Cc1cccc(Cl)c1)c1cccc(Cl)c1)C(=O)N[C@H](C=O)C[C@@H]1CCNC1=O] |
| C283 | 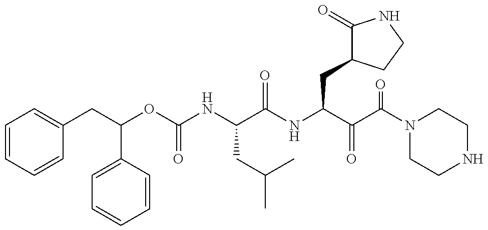 [SMILES: CC(C)C[C@H](NC(=O)OC(Cc1ccccc1)c1ccccc1)C(=O)N[C@@H](C[C@@H]1CCNC1=O)C(=O)C(=O)N1CCNCC1] |
| C284 | 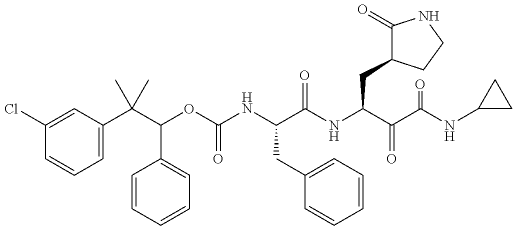 [SMILES: CC(C)(c1cccc(Cl)c1)C(OC(=O)N[C@@H](Cc1ccccc1)C(=O)N[C@@H](C[C@@H]1CCNC1=O)C(=O)C(=O)NC1CC1)c1ccccc1] |
| C285 | 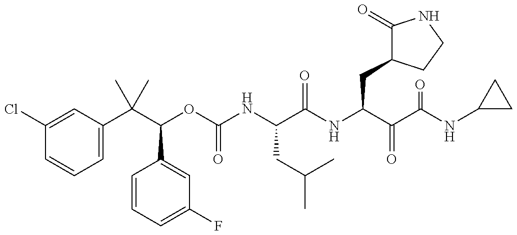 [SMILES: CC(C)C[C@H](NC(=O)O[C@@H](c1cccc(F)c1)C(C)(C)c1cccc(Cl)c1)C(=O)N[C@@H](C[C@@H]1CCNC1=O)C(=O)C(=O)NC1CC1] |
| C286 | 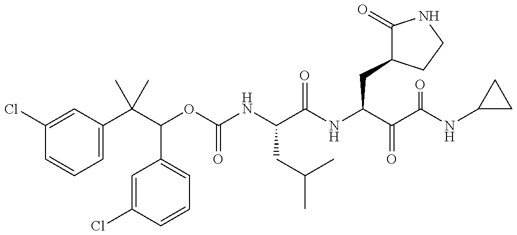 [SMILES: CC(C)C[C@H](NC(=O)OC(c1cccc(Cl)c1)C(C)(C)c1cccc(Cl)c1)C(=O)N[C@@H](C[C@@H]1CCNC1=O)C(=O)C(=O)NC1CC1] |
| C287 | 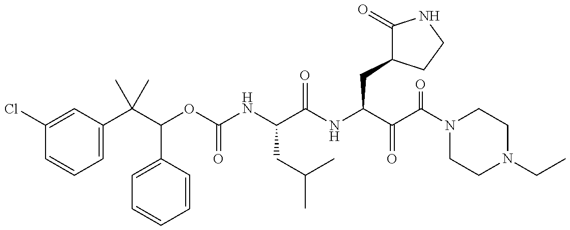 [SMILES: CCN1CCN(C(=O)C(=O)[C@H](C[C@@H]2CCNC2=O)NC(=O)[C@H](CC(C)C)NC(=O)OC(c2ccccc2)C(C)(C)c2cccc(Cl)c2)CC1] |
| C288 | 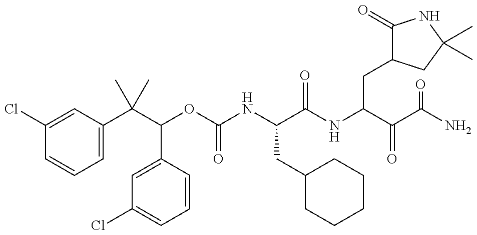 [SMILES: CC1(C)CC(CC(NC(=O)[C@H](CC2CCCCC2)NC(=O)OC(c2cccc(Cl)c2)C(C)(C)c2cccc(Cl)c2)C(=O)C(N)=O)C(=O)N1] |
| C289 | 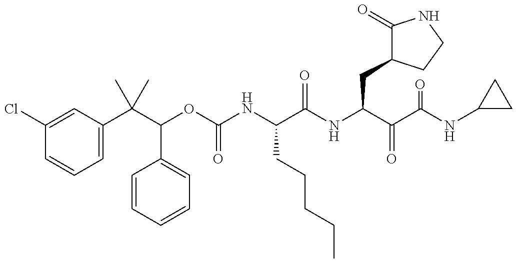 [SMILES: CCCCC[C@H](NC(=O)OC(c1ccccc1)C(C)(C)c1cccc(Cl)c1)C(=O)N[C@@H](C[C@@H]1CCNC1=O)C(=O)C(=O)NC1CC1] |
| C290 | 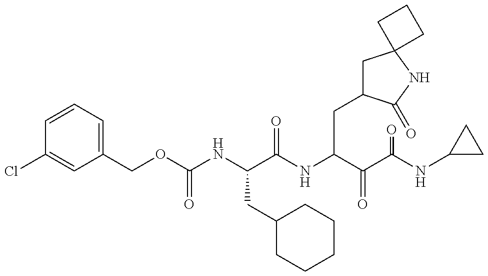 [SMILES: O=C(N[C@@H](CC1CCCCC1)C(=O)NC(CC1CC2(CCC2)NC1=O)C(=O)C(=O)NC1CC1)OCc1cccc(Cl)c1] |
| C291 | 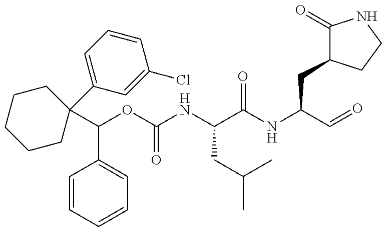 [SMILES: CC(C)C[C@H](NC(=O)OC(c1ccccc1)C1(c2cccc(Cl)c2)CCCCC1)C(=O)N[C@H](C=O)C[C@@H]1CCNC1=O] |
| C292 | 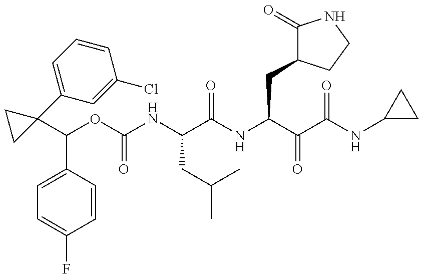 [SMILES: CC(C)C[C@H](NC(=O)OC(c1ccc(F)cc1)C1(c2cccc(Cl)c2)CC1)C(=O)N[C@@H](C[C@@H]1CCNC1=O)C(=O)C(=O)NC1CC1] |
| C293 | 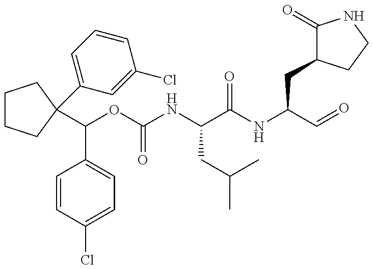 [SMILES: CC(C)C[C@H](NC(=O)OC(c1ccc(Cl)cc1)C1(c2cccc(Cl)c2)CCCC1)C(=O)N[C@H](C=O)C[C@@H]1CCNC1=O] |
| C294 | 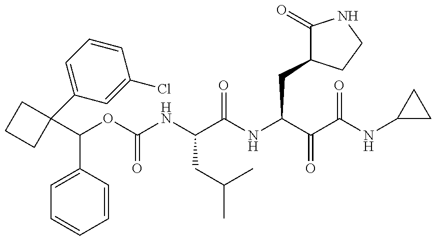 [SMILES: CC(C)C[C@H](NC(=O)OC(c1ccccc1)C1(c2cccc(Cl)c2)CCC1)C(=O)N[C@@H](C[C@@H]1CCNC1=O)C(=O)C(=O)NC1CC1] |
| C295 | 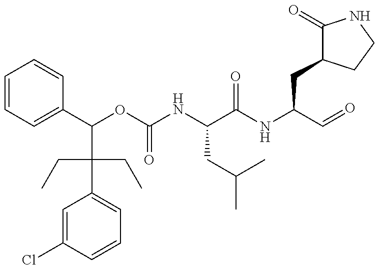 [SMILES: CCC(CC)(c1cccc(Cl)c1)C(OC(=O)N[C@@H](CC(C)C)C(=O)N[C@H](C=O)C[C@@H]1CCNC1=O)c1ccccc1] |
| C296 | 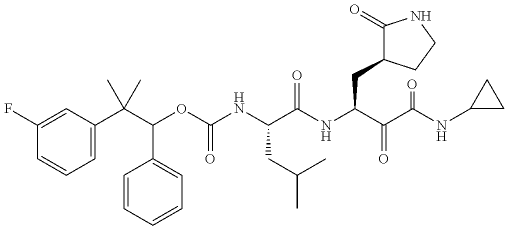 [SMILES: CC(C)C[C@H](NC(=O)OC(c1ccccc1)C(C)(C)c1cccc(F)c1)C(=O)N[C@@H](C[C@@H]1CCNC1=O)C(=O)C(=O)NC1CC1] |
| C297 | 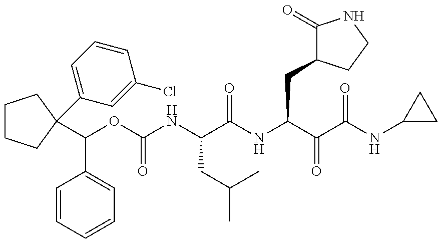 [SMILES: CC(C)C[C@H](NC(=O)OC(c1ccccc1)C1(c2cccc(Cl)c2)CCCC1)C(=O)N[C@@H](C[C@@H]1CCNC1=O)C(=O)C(=O)NC1CC1] |
| C298 | 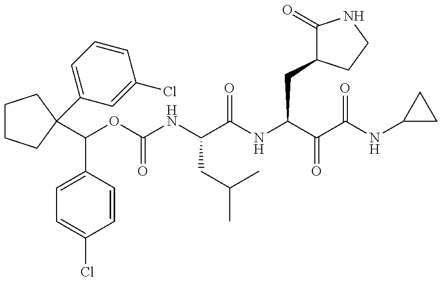 [SMILES: CC(C)C[C@H](NC(=O)OC(c1ccc(Cl)cc1)C1(c2cccc(Cl)c2)CCCC1)C(=O)N[C@@H](C[C@@H]1CCNC1=O)C(=O)C(=O)NC1CC1] |
| C299 | 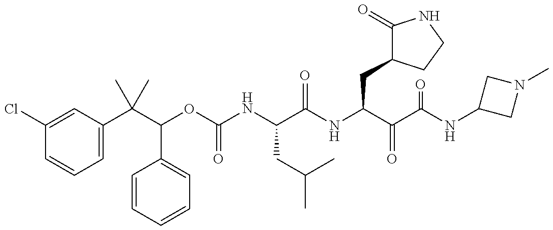 [SMILES: CC(C)C[C@H](NC(=O)OC(c1ccccc1)C(C)(C)c1cccc(Cl)c1)C(=O)N[C@@H](C[C@@H]1CCNC1=O)C(=O)C(=O)NC1CN(C)C1] |
| C300 | 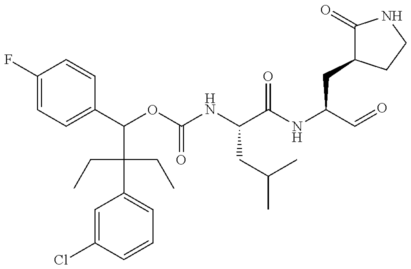 [SMILES: CCC(CC)(c1cccc(Cl)c1)C(OC(=O)N[C@@H](CC(C)C)C(=O)N[C@H](C=O)C[C@@H]1CCNC1=O)c1ccc(F)cc1] |
| C301 | 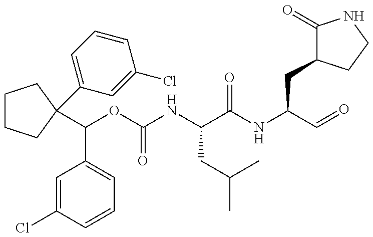 [SMILES: CC(C)C[C@H](NC(=O)OC(c1cccc(Cl)c1)C1(c2cccc(Cl)c2)CCCC1)C(=O)N[C@H](C=O)C[C@@H]1CCNC1=O] |
| C302 | 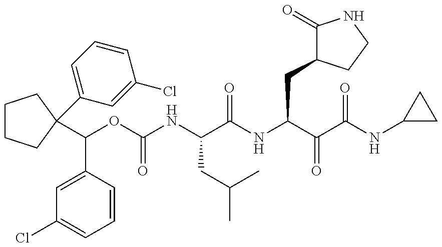 [SMILES: CC(C)C[C@H](NC(=O)OC(c1cccc(Cl)c1)C1(c2cccc(Cl)c2)CCCC1)C(=O)N[C@@H](C[C@@H]1CCNC1=O)C(=O)C(=O)NC1CC1] |
| C303 | 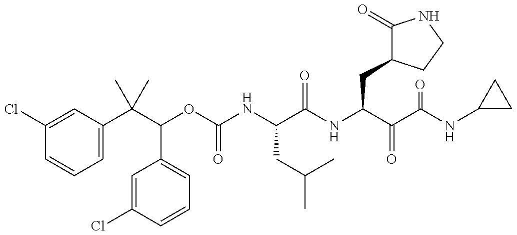 [SMILES: CC(C)C[C@H](NC(=O)OC(c1cccc(Cl)c1)C(C)(C)c1cccc(Cl)c1)C(=O)N[C@@H](C[C@@H]1CCNC1=O)C(=O)C(=O)NC1CC1] |
| C304 | 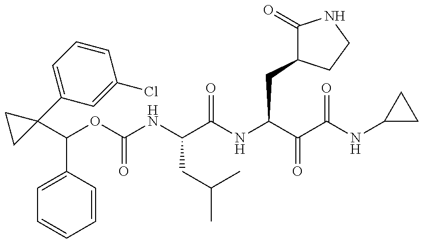 [SMILES: CC(C)C[C@H](NC(=O)OC(c1ccccc1)C1(c2cccc(Cl)c2)CC1)C(=O)N[C@@H](C[C@@H]1CCNC1=O)C(=O)C(=O)NC1CC1] |
| C305 | 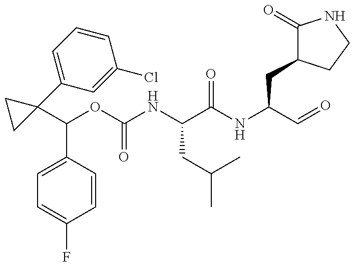 [SMILES: CC(C)C[C@H](NC(=O)OC(c1ccc(F)cc1)C1(c2cccc(Cl)c2)CC1)C(=O)N[C@H](C=O)C[C@@H]1CCNC1=O] |
| C306 | 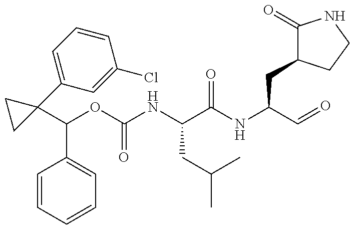 [SMILES: CC(C)C[C@H](NC(=O)OC(c1ccccc1)C1(c2cccc(Cl)c2)CC1)C(=O)N[C@H](C=O)C[C@@H]1CCNC1=O] |
| C307 | 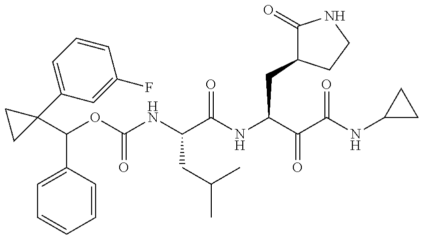 [SMILES: CC(C)C[C@H](NC(=O)OC(c1ccccc1)C1(c2cccc(F)c2)CC1)C(=O)N[C@@H](C[C@@H]1CCNC1=O)C(=O)C(=O)NC1CC1] |
| C308 | 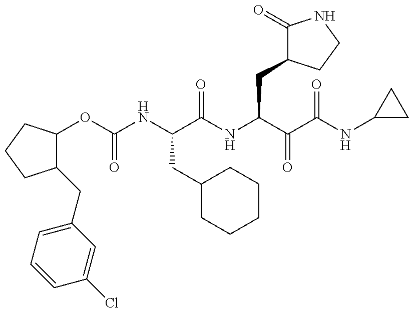 [SMILES: O=C(N[C@@H](CC1CCCCC1)C(=O)N[C@@H](C[C@@H]1CCNC1=O)C(=O)C(=O)NC1CC1)OC1CCCC1Cc1cccc(Cl)c1] |
| C309 | 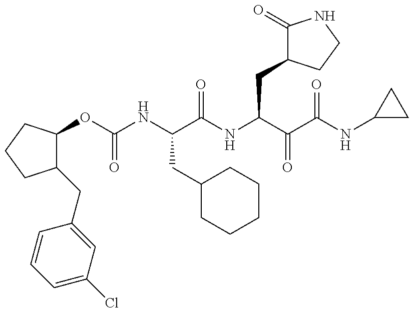 [SMILES: O=C(N[C@@H](CC1CCCCC1)C(=O)N[C@@H](C[C@@H]1CCNC1=O)C(=O)C(=O)NC1CC1)O[C@@H]1CCCC1Cc1cccc(Cl)c1] |
| C310 | 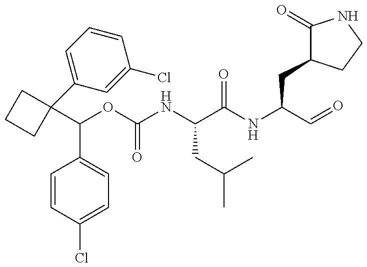 [SMILES: CC(C)C[C@H](NC(=O)OC(c1ccc(Cl)cc1)C1(c2cccc(Cl)c2)CCC1)C(=O)N[C@H](C=O)C[C@@H]1CCNC1=O] |
| C311 | 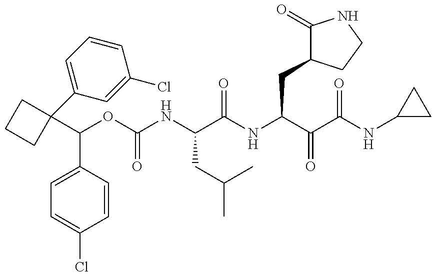 [SMILES: CC(C)C[C@H](NC(=O)OC(c1ccc(Cl)cc1)C1(c2cccc(Cl)c2)CCC1)C(=O)N[C@@H](C[C@@H]1CCNC1=O)C(=O)C(=O)NC1CC1] |

The compounds disclosed herein can be useful as inhibitors of norovirus or coronavirus replication in biological samples or in a patient. These compounds can also be useful in reducing the amount of noroviruses or coronaviruses (viral titer) in a biological sample or in a patient. They can also be useful for therapeutic and prophylactic treatment of infections caused by the noroviruses or coronaviruses in a biological sample or in a patient.

### Pharmaceutically Acceptable Salts

The compounds described herein can exist in free form, or, where appropriate, as salts. Those salts that are pharmaceutically acceptable are of particular interest since they are useful in administering the compounds described below for medical purposes. Salts that are not pharmaceutically acceptable are useful in manufacturing processes, for isolation and purification purposes, and in some instances, for use in separating stereoisomeric forms of the compounds of the disclosure or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Pharmaceutical Compositions

The compounds described herein can be formulated into pharmaceutical compositions that further comprise a pharmaceutically acceptable carrier, diluent, adjuvant or vehicle. In embodiments, the present disclosure relates to a pharmaceutical composition comprising a compound described above or salt thereof, and a pharmaceutically acceptable carrier, diluent, adjuvant or vehicle. In embodiments, the pharmaceutical composition comprises a safe and effective amount of a compound as disclosed herein or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

An “effective amount” includes a “therapeutically effective amount” and a “prophylactically effective amount”. The term “therapeutically effective amount” refers to an amount effective in treating and/or ameliorating a norovirus or coronavirus virus infection in a patient. The term “prophylactically effective amount” refers to an amount effective in preventing and/or substantially lessening the chances or the size of norovirus or coronavirus virus infection outbreak.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this disclosure. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Formulations for Pulmonary Delivery

In some embodiments, the pharmaceutical compositions disclosed herein are adapted to be administered to the lower respiratory tract (e.g., the lungs) directly through the airways by inhalation. Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose or starch. Inhalable dry powder compositions may be presented in capsules and cartridges of gelatin or a like material, or blisters of laminated aluminum foil for use in an inhaler or insufflators. Each capsule or cartridge may generally contain e.g., from about 10 mg to about 100 g of each active compound. Alternatively, the composition may be presented without excipients.

The inhalable compositions may be packaged for unit dose or multi-dose delivery. For example, the compositions can be packaged for multi-dose delivery in a manner analogous to that described in GB 2242134, U.S. Pat. Nos. 6,632,666, 5,860,419, 5,873,360, and 5,590,645 (all illustrating the “Diskus” device), or GB2i78965, GB2129691, GB2169265, U.S. Pat. Nos. 4,778,054, 4,811,731 and 5,035,237 (which illustrate the “Diskhaler” device), or EP 69715 (“Turbuhaler” device), or GB 2064336 and U.S. Pat. No. 4,353,656 (“Rotahaler” device).

Spray compositions for topical delivery to the lung by inhalation may be formulated as aqueous solutions or suspensions or as aerosols delivered from pressurized packs, such as a metered dose inhaler (MDI), with the use of a suitable liquefied propellant, including hydrofluoroalkanes such as dichlorodifluoromethane, trichlorofluoromethane, dichlorotetrafluoroethane, and especially 1,1,1,2-tetrafluoroethane, 1,1,1,2,3,3,3-heptafluoro-n-propane and mixtures thereof. Aerosol compositions suitable for inhalation can be presented either as suspensions or as solutions.

Medicaments for administration by inhalation typically have a controlled particle size. The optimum particle size for inhalation into the bronchial system is usually about 1 to about 10 μm, and in some embodiments, from about 2 to about 5 μm. Particles having a size above about 20 μm are generally too large when inhaled to reach the small airways. To achieve these particle sizes the particles of the active ingredient may be subjected to a size reducing process such as micronization. The desired size fraction may be separated out by air classification or sieving. Preferably, the particles will be crystalline.

Intranasal sprays may be formulated with aqueous or non-aqueous vehicles with the addition of agents such as thickening agents, buffer salts or acid or alkali to adjust the pH, isotonic adjusting agents or anti-oxidants.

Solutions for inhalation by nebulization may be formulated with an aqueous vehicle with the addition of agents such as acid or alkali, buffer salts, isotonic adjusting agents or antimicrobial agents. They may be sterilized by filtration or heating in an autoclave, or presented as a non-sterile product. Nebulizers supply the aerosol as a mist created from an aqueous formulation.

In some embodiments, the pharmaceutical compositions disclosed herein can be formulated with supplementary active ingredients.

In some embodiments, the pharmaceutical composition disclosed herein is administered from a dry powder inhaler. In other embodiments, the pharmaceutical composition disclosed herein is administered by an aerosol dispensing device, optionally in conjunction with an inhalation chamber such as the “Volumatic”® inhalation chamber.

The carrier can be a solvent or dispersion medium containing, for example, water, ethanol, polyol (for example, glycerol, propylene glycol and liquid polyethylene glycol, and the like), suitable mixtures thereof, and/or vegetable oils. The proper fluidity can be maintained, for example, by the use of a coating such as, for example, lecithin, by the maintenance of the required particle size in the case of dispersion and by the use of surfactants. Preventing the action of microorganisms in the compositions disclosed herein is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, a pharmaceutical composition can be within a matrix which controls the release of the composition. In some embodiments, the matrix can comprise lipid, polyvinyl alcohol, polyvinyl acetate, polycaprolactone, poly(glycolic)acid, poly(lactic)acid, polycaprolactone, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and combinations thereof and other polymers such as those disclosed, for example, in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety. In these embodiments, the matrix sustainedly releases the drug.

Pharmaceutically acceptable carriers and/or diluents may also include any solvents, dispersion media, coatings, antibacterials and/or antifungals, isotonic and absorption delaying agents and the like. The use of such media and agents for pharmaceutically active substances is well known in the art. Except insofar as any conventional medium or agent is incompatible with the active ingredient, use thereof in the pharmaceutical compositions is contemplated.

The pharmaceutical compositions can be formulated for administration in accordance with conventional techniques. See, e.g., Remington, The Science and Practice of Pharmacy (20th Ed. 2000). For example, the intranasal pharmaceutical compositions of the present disclosure can be formulated as an aerosol (this term includes both liquid and dry powder aerosols). Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles (e.g., lyophilized, freeze dried, etc.) can likewise be produced with any solid particulate medicament aerosol generator, by techniques known in the pharmaceutical art. As another example, the pharmaceutical compositions can be formulated as an on-demand dissolvable form, which provides a lyophilized portion of the pharmaceutical composition and a dissolving solution portion of the pharmaceutical composition.

In some embodiments, the pharmaceutical composition is in the form of an aqueous suspension, which can be prepared from solutions or suspensions. With respect to solutions or suspensions, dosage forms can be comprised of micelles of lipophilic substances, liposomes (phospholipid vesicles/membranes) and/or a fatty acid (e.g., palmitic acid). In particular embodiments, the pharmaceutical composition is a solution or suspension that is capable of dissolving in the fluid secreted by mucous membranes of the epithelium of the tissue to which it is administered, applied and/or delivered, which can advantageously enhance absorption.

The pharmaceutical composition can be an aqueous solution, a nonaqueous solution or a combination of an aqueous and nonaqueous solution. Suitable aqueous solutions include, but are not limited to, aqueous gels, aqueous suspensions, aqueous microsphere suspensions, aqueous microsphere dispersions, aqueous liposomal dispersions, aqueous micelles of liposomes, aqueous microemulsions, and any combination of the foregoing, or any other aqueous solution that can dissolve in the fluid secreted by the mucosal membranes of the nasal cavity. Exemplary nonaqueous solutions include, but are not limited to, nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing, or any other nonaqueous solution that can dissolve or mix in the fluid secreted by mucosal membranes.

Examples of powder formulations include, without limitation, simple powder mixtures, micronized powders, freeze dried powder, lyophilized powder, powder microspheres, coated powder microspheres, liposomal dispersions, and any combination of the foregoing. Powder microspheres can be formed from various polysaccharides and celluloses, which include without limitation starch, methylcellulose, xanthan gum, carboxymethylcellulose, hydroxypropyl cellulose, carbomer, alginate polyvinyl alcohol, acacia, chitosans, and any combination thereof.

In particular embodiments, the composition is one that is at least partially, or even substantially (e.g., at least 80%, 90%, 95% or more) soluble in the fluids that are secreted by mucosa so as to facilitate absorption. Alternatively or additionally, the composition can be formulated with a carrier and/or other substances that foster dissolution of the agent within secretions, including without limitation fatty acids (e.g., palmitic acid), gangliosides (e.g., GM-1), phospholipids (e.g., phosphatidylserine), and emulsifiers (e.g., polysorbate 80).

Those skilled in the art will appreciate that for intranasal administration or delivery, because the volume of the pharmaceutical composition administered is generally small, nasal secretions may alter the pH of the administered dose since the range of pH in the nasal cavity can be as wide as 5 to 8. Such alterations can affect the concentration of un-ionized drug available for absorption. Accordingly, in representative embodiments, the pharmaceutical composition further comprises a buffer to maintain or regulate pH in situ. Typical buffers include, but are not limited to, ascorbate, acetate, citrate, prolamine, carbonate, and phosphate buffers.

In embodiments, the pH of the pharmaceutical composition is selected so that the internal environment of the mucosal tissue after administration is on the acidic to neutral side, which (1) can provide the active compound in an un-ionized form for absorption, (2) prevents growth of pathogenic bacteria, which is more likely to occur in an alkaline environment, and (3) reduces the likelihood of irritation of the mucosa.

For liquid and powder sprays or aerosols, the pharmaceutical composition can be formulated to have any suitable and desired particle or droplet size. In illustrative embodiments, the majority and/or the mean size of the particles or droplets range from equal to or greater than about 1, 2.5, 5, 10, 15 or 20 microns and/or equal to or less than about 25, 30, 40, 45, 50, 60, 75, 100, 125, 150, 175, 200, 225, 250, 275, 300, 325, 350, 375, 400, or 425 microns (including all combinations of the foregoing). Representative examples of suitable ranges for the majority and/or mean particle or droplet size include, without limitation, from about 5 to 100 microns, from about 10 to 60 microns, from about 175 to 325 microns, and from about 220 to 300 microns which facilitate the deposition of a safe and effective amount of the active compound, for example, in the nasal cavity (e.g., in the upper third of the nasal cavity, the superior meatus, the olfactory region and/or the sinus region to target the olfactory neural pathway). In general, particles or droplets smaller than about 5 microns will be deposited in the trachea or even the lung, whereas particles or droplets that are about 50 microns or larger generally do not reach the nasal cavity and are deposited in the anterior nose.

International patent publication WO 2005/023335 (Kurve Technology, Inc.) describes particles and droplets having a diameter size suitable for the practice of representative embodiments of pharmaceutical compositions disclosed herein. In particular embodiments, the particles or droplets have a mean diameter of about 5 to 30 microns, about 10 to 20 microns, about 10 to 17 microns, about 10 to 15 microns, about 12 to 17 microns, about 10 to 15 microns or about 10 to 12 microns. The particles can “substantially” have a mean diameter or size as described herein, i.e., at least about 50%, 60%, 70%, 80%, 90% or 95 or more of the particles are of the indicated diameter or size range.

The pharmaceutical composition can be delivered as a nebulized or atomized liquid having a droplet size as described above.

According to particular embodiments of this disclosure that comprise methods of intranasal delivery, it can be desirable to prolong the residence time of the pharmaceutical composition in the nasal cavity (e.g., in the upper third of the nasal cavity, the superior meatus, the olfactory region and/or in the sinus region), for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbomer, polyvinyl alcohol, alginate, acacia, chitosans and/or cellulose (e.g., methyl or propyl; hydroxyl or carboxy; carboxymethyl or hydroxylpropyl), which are agents that enhance residence time in the nasal cavity. As a further approach, increasing the viscosity of the formulation can also provide a means of prolonging contact of the agent with the nasal epithelium. The pharmaceutical composition can be formulated as a nasal emulsion, ointment or gel, which offers advantages for local application because of their viscosity.

Moist and highly vascularized membranes can facilitate rapid absorption; consequently, the pharmaceutical composition can optionally comprise a humectant, particularly in the case of a gel-based composition so as to assure adequate intranasal moisture content. Examples of suitable humectants include but are not limited to glycerin or glycerol, mineral oil, vegetable oil, membrane conditioners, soothing agents, and/or sugar alcohols (e.g., xylitol, sorbitol; and/or mannitol). The concentration of the humectant in the pharmaceutical composition will vary depending upon the agent selected and the formulation.

The pharmaceutical composition can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of about 2% by weight or less.

The pharmaceutical compositions described herein can optionally contain an odorant, e.g., as described in EP 0 504 263 B1, to provide a sensation of odor, to aid in inhalation of the composition so as to promote delivery to the olfactory region and/or to trigger transport by the olfactory neurons.

As another option, the composition can comprise a flavoring agent, e.g., to enhance the taste and/or acceptability of the composition to the subject.

### Porous Particles for Pulmonary Administration

In some embodiments, the particles are porous, so that they have an appropriate density to avoid deposition in the back of the throat when administered via an inhaler. The combination of relatively large particle size and relatively low density avoids phagocytosis in the lungs, provides appropriately targeted delivery, avoids systemic delivery of the components, and provides a high concentration of the components in the lung.

Representative methods for preparing such particles, and for delivering such particles, are described, for example, in U.S. Pat. No. 7,384,649, entitled, “Particulate compositions for pulmonary delivery,” U.S. Pat. No. 7,182,961, entitled “Particulate compositions for pulmonary delivery,” U.S. Pat. No. 7,146,978, entitled, “Inhalation device and method,” U.S. Pat. No. 7,048,908, entitled “Particles for inhalation having sustained release properties,” U.S. Pat. No. 6,956,021, entitled “Stable spray-dried protein formulations,” U.S. Pat. No. 6,766,799, entitled “Inhalation device,” and U.S. Pat. No. 6,732,732, entitled “Inhalation device and method.”

Additional patents disclosing such particles include U.S. Pat. No. 7,279,182, entitled “Formulation for spray-drying large porous particles,” U.S. Pat. No. 7,252,840, entitled “Use of simple amino acids to form porous particles,” U.S. Pat. No. 7,032,593, entitled “Inhalation device and method,” U.S. Pat. No. 7,008,644, entitled “Method and apparatus for producing dry particles,” U.S. Pat. No. 6,848,197, entitled “Control of process humidity to produce large, porous particles,” and U.S. Pat. No. 6,749,835, entitled “Formulation for spray-drying large porous particles.”

U.S. Pat. No. 7,678,364, entitled “Particles for inhalation having sustained release properties,” discloses methods for delivering particles to the pulmonary system comprising: administering to the respiratory tract of a patient in need of treatment, prophylaxis or diagnosis a safe and effective amount of a dry powder comprising: a) a multivalent metal cation which is complexed with a therapeutic, prophylactic or diagnostic agent; b) a pharmaceutically acceptable carrier; and c) a multivalent metal cation-containing component wherein the dry powder is spray-dried and has a total amount of multivalent metal cation which is about 10% w/w or more of the total weight of the agent, a tap density of about 0.4 g/cm³ or less, a median geometric diameter of from about 5 micrometers to about 30 micrometers and an aerodynamic diameter of from about 1 to about 5 microns.

The amount of the compounds described herein, or salts thereof, present in the particles can range from about 0.1 weight % to about 95 weight %, though in some cases, can even be as high as 100%. For example, from about 1 to about 50%, such as from about 5 to about 30%. Particles in which the compound is distributed throughout a particle can be preferred.

In some embodiments, the particles include a surfactant other than the phospholipids described above. As used herein, the term “surfactant” refers to any agent which preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to particles, they tend to present moieties to the external environment that do not attract similarly-coated particles, thus reducing particle agglomeration. Surfactants may also promote absorption of a therapeutic or diagnostic agent and increase bioavailability of the agent.

Suitable surfactants which can be employed in fabricating the particles disclosed herein include but are not limited to hexadecanol; fatty alcohols such as polyethylene glycol (PEG); polyoxyethylene-9-lauryl ether; a surface active fatty acid, such as palmitic acid or oleic acid; glycocholate; surfactin; a poloxamer; a sorbitan fatty acid ester such as sorbitan trioleate (Span 85); Tween® 80 and tyloxapol.

The surfactant can be present in the particles in an amount ranging from about 0 to about 5 weight %. Preferably, it can be present in the particles in an amount ranging from about 0.1 to about 1.0 weight %.

Particles that have a tap density less than about 0.4 g/cm³, median diameters of at least about 5 μm, and an aerodynamic diameter of from about 1 μm to about 5 μm, or from about 1 μm to about 3 μm, are more capable of escaping inertial and gravitational deposition in the oropharyngeal region, and are targeted to the airways or the deep lung. The use of larger, more porous particles is advantageous since they are able to aerosolize more efficiently than smaller, denser aerosol particles such as those currently used for inhalation therapies.

### Liposomal Delivery

The compositions described herein are advantageously delivered to the lungs, so as to provide the compounds at the site of an actual or potential norovirus or coronavirus infection. This can be accomplished by pulmonary delivery via metered-dose inhalers or other pulmonary delivery devices, and also by lodging particles in the capillary beds surrounding the alveoli in the lungs.

Nanocarriers, such as liposomes, including small unilamellar vesicles, show several advantages over other conventional approaches for delivering drugs to the lungs, including prolonged drug release and cell-specific targeted drug delivery. Nano-sized drug carriers can also be advantageous for delivering poorly water soluble drugs, and certain of the compounds described herein are poorly water-soluble. Additional advantages include their ability to provide controlled release, protection from metabolism and degradation, decreased drug toxicity and targeting capabilities.

The liposomes (preferably unilamellar vesicles) have a size less than 200 nm as measured by dynamic light scattering, and preferably characterized by being comprised of chemically pure synthetic phospholipids, most preferably having aliphatic side chains of a length of at least 16 carbons, and containing one or more of the compounds described herein, or a pharmaceutically acceptable salt thereof, sufficient to preferentially deliver (i.e., target) a quantity of the compounds thereof to the capillary beds surrounding the alveoli. Vesicle diameter can be measured, for example, by dynamic light scattering using a helium-neon 100 mW NEC gas laser and a Malvern K7027 correlator, ideally with at least two or three measurements made for each for each size determination.

The expression “chemically pure phospholipids” is meant to define phospholipids which are essentially free of deleterious detergent moieties and impurities which cause aggregation of small unilamellar vesicles (SUVs) formed therefrom, and which are more than 97% pure. Preferably, the liposomes have a diameter predominantly of from about 50 to about 160 nm, are essentially neutral in charge, and incorporate phospholipids having a side chain length of from 16 to 18 carbon atoms. More preferably, the liposomes are prepared from distearoyl phosphatidylcholine (DSPC) and include cholesterol (most preferably in an amount of from 10 to 50% of total lipid) as a vesicle stabilizer.

It can also be advantageous that the liposomes have a melting point above body temperature (i.e., above 37° C.). For this reason, it can be advantageous to use pure phospholipids, preferably ones that are saturated, and have a carbon chain length of at least 16 carbons, preferably between 16 and 18 carbons. Distearoylphosphatidyl choline (DSPC) is a preferred phospholipid.

Cholesterol helps to stabilize the liposomes, and is preferably added in a sufficient amount to provide liposome stability. Most preferably, the liposomes further comprise a pegylated phospholipid, such as DSPEPEG. The method involves introducing into a patient's bloodstream an amount of liposomes, of a size of less than 200 nm (preferably unilamellar vesicles) and preferably characterized by being comprised of chemically pure synthetic phospholipids, most preferably having aliphatic side chains of a length of at least 16 carbons, and containing the compounds described herein, or a pharmaceutically acceptable salt or prodrug thereof, sufficient to preferentially deliver (i.e., target) a quantity of the compounds to the capillary beds in the lungs, surrounding the alveoli.

The compounds described herein can be combined with other anti-norovirus or anti-coronavirus agents. Such additional agents can also be present in the liposomes, can be present in different liposomes, or can be co-administered via a different route.

The liposomes include one or more of the compounds described herein, or a pharmaceutically acceptable salt thereof, and can optionally include other anti-norovirus or anti-coronavirus agents. The liposomes can be prepared by dissolving the phospholipid and cholesterol in an appropriate organic solvent, such as chloroform, and evaporating the solvent to form a lipid film. If an ionophore is employed to load the compounds described herein into the liposomes, the ionophore may be added to the lipid solution before evaporation. The dried lipid film is then rehydrated in an appropriate aqueous phase, such as phosphate-buffered saline or other physiologically appropriate solution. Water-soluble drugs or therapeutic agents may be contained in the hydrating solution, although if remote loading is desired a loading agent such as a chelating agent described above may be added to the hydrating solution to be encapsulated within the inner aqueous space of the liposome.

Upon the addition of the hydrating solution, liposomes of varying size spontaneously form and encapsulate a portion of the aqueous phase. Thereafter, the liposomes and suspending aqueous solution are subjected to a shear force such as extrusion, sonication, or processing through a homogenizer according to the method described in U.S. Pat. No. 4,753,788; to produce vesicles within the specified size.

The liposomes can then be processed to remove undesirable compounds from the suspending solution, for example, un-encapsulated drug, which may be accomplished through processes such as gel chromatography or ultrafiltration.

The use of liposomes in dry powder aerosols for targeted lung delivery is described, for example, in Willis et al., Lung, June 2012, 190(3):251-262. One advantage is that the phospholipids used to prepare the liposomes are similar to endogenous lung surfactant.

### Routes of Administration and Dosages

The compounds and pharmaceutically acceptable compositions described above can be administered to humans and other animals orally, rectally, parenterally, intracisternally, intravaginally, intraperitoneally, topically (as by powders, ointments, or drops), bucally, as an oral or nasal spray, to the pulmonary system, such as by using an inhaler, such as a metered dose inhaler (MDI), or the like, depending on the severity of the infection being treated. In some embodiments, the compound or composition disclosed herein is administered orally, via inhalation, or intravenously.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topical application also includes the use of transdermal patches.

For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

### Methods of Treatment

Provided herein are uses of a compound described herein as a therapeutic agent. The compounds described herein or pharmaceutically acceptable salts thereof can be used to reduce viral titer in a biological sample (e.g. an infected cell culture) or in humans (e.g. lung viral titer in a patient). The compounds described herein or pharmaceutically acceptable salts thereof can be used in methods of treating viral infections. Non-limiting examples of viral infections which can be treated with the compounds described herein or their pharmaceutically acceptable salts include coronavirus infections, calicivirus infections, and picornavirus infections.

Non-limiting examples of calicivirus infections include norovirus mediated conditions and norovirus infection. The terms “norovirus mediated condition”, “norovirus infection”, and “norovirus”, as used herein, are used interchangeably to mean the disease caused by an infection with a norovirus.

Noroviruses are infectious viruses that cause gastroenteritis in mammals. Noroviruses are RNA viruses of the family Caliciviridae, which comprises seven genogroups: GI, GII, GIII, GIV, GV, GVI, and GVII. Genogroup II, the most prevalent human genogroup, presently contains 19 genotypes. Genogroups I, II and IV infect humans, whereas genogroup III infects bovine species, and genogroup V has recently been isolated in mice. The two groups most associated with gastroenteritis in humans are genogroup I (GI), which includes Norwalk virus, Desert Shield virus and Southampton virus; and genogroup II (GII), which includes Bristol virus, Lordsdale virus, Toronto virus, Mexico virus, Hawaii virus and Snow Mountain virus.

In some embodiments, the compounds used herein are for treatment of noroviruses which are associated with gastroenteritis. In some embodiments, noroviruses are associated with Norwalk virus. In some embodiments, noroviruses are associated with HuNV GGII.4.

In some embodiments, the compounds disclosed herein can be used in the treatment of norovirus, wherein the compound binds to free virus, or inhibits a norovirus protease. In some cases, the compound can target both (free virus and protease).

In humans, common symptoms of norovirus are nausea, vomiting, watery diarrhea, abdominal pain, and in some cases, loss of taste. Norovirus can establish a long term infection in people who are immunocompromised. In severe cases, persistent infections can lead to norovirus-associated enteropathy, intestinal villous atrophy, and malabsorption. Norovirus-associated gastroenteritis is also called “winter vomiting bug”.

A person usually develops symptoms of gastroenteritis 12 to 48 hours after being exposed to norovirus. General lethargy, weakness, muscle aches, headaches, and low-grade fevers may occur.

The terms “coronavirus mediated condition” and “coronavirus infection” as used herein, are used interchangeably to mean the disease caused by an infection with a coronavirus. Non-limiting examples of coronaviruses include severe acute respiratory syndrome-related coronavirus (SARS), Middle East respiratory syndrome-related coronavirus (MERS), and SARS-CoV-2 virus (also known as 2019-nCoV, or Wuhan coronavirus). Non-limiting examples of coronavirus mediated conditions or coronavirus infections include SARS, MERS, and COVID-19.

Coronaviruses are a family of viruses that cause diseases in mammals and birds. Coronaviruses are in the subfamily Orthocoronavirinae in the family Coronaviridae, in the order Nidovirales. There are four main genera of coronaviruses, known as alpha, beta, gamma, and delta. Coronaviruses that affect humans include Human coronavirus 229E (HCoV-229E), Human coronavirus OC43 (HCoV-OC43), Severe acute respiratory syndrome-related coronavirus (SARS-CoV), Human coronavirus NL63 (HCoV-NL63, New Haven coronavirus), Human coronavirus HKU1, Middle East respiratory syndrome-related coronavirus (MERS-CoV, previously known as novel coronavirus 2012 and HCoV-EMC), and SARS-CoV-2 (also known as 2019-nCoV and Wuhan coronavirus).

In humans, coronaviruses cause respiratory infections, including the common cold, which are typically mild, though rarer forms such as SARS, MERS and SARS-CoV-2 (the cause of the 2019-20 COVID-19 outbreak) can be lethal. Symptoms vary in other species: in chickens, they cause an upper respiratory disease, while in cows and pigs coronaviruses cause diarrhea. There are no vaccines or antiviral drugs to prevent or treat human coronavirus infections. The coronaviruses HCoV-229E, -NL63, -OC43, and -HKU1 continually circulate in the human population and cause respiratory infections in adults and children worldwide

In some embodiments, the compounds used herein are for treatment of alphacoronaviruses or betacoronaviruses. In some cases, the compounds used herein are for treatment of alphacoronaviruses. Non-limiting examples of alphacoronaviruses include HCoV-229E and HCoV-NL63. In some embodiments, the compounds used herein are for treatment of betacoronaviruses. Non-limiting examples of betacoronaviruses are HCoV-HKU1, HCoV-OC43, Middle East respiratory syndrome coronavirus (MERS-CoV), the severe acute respiratory syndrome coronavirus (SARS-CoV), and SARS-CoV-2. In some embodiments, the compounds used herein are for treatment of coronaviruses which are associated with SARS, MERS, and COVID-19. In some embodiments, coronaviruses are associated with SARS. In some embodiments, coronaviruses are associated with MERS. In some embodiments, coronaviruses are associated with COVID-19.

In some embodiments, the compounds disclosed herein can be used in the treatment of coronavirus, wherein the compound binds to free virus, or inhibits a coronavirus protease. In some cases, the compound can target both (free virus and protease).

In humans, common symptoms of coronavirus are fever, cough, shortness of breath, and myalgia.

Non-limiting examples of picornavirus infections include rhinovirus mediated conditions and rhinovirus infections. The terms “rhinovirus mediated condition” and “rhinovirus infection” as used herein, are used interchangeably to mean the disease caused by an infection with a rhinovirus.

Picornaviruses infect both humans and animals, can cause severe paralysis (paralytic poliomyelitis), aseptic meningitis, hepatitis, pleurodynia, myocarditis, skin rashes, and colds; although asymptomatic infection is common. Several medically important genera are members of this family, such as enterovirus (including poliovirus (PV), rhinoviruses, and human enteroviruses (e.g. coxsackie viruses)); hepatovirus which includes hepatitis A virus (HAV); and aphthoviruses which include the foot- and mouth disease virus (FMDV). Rhinoviruses are recognized as the principle cause of the common cold in humans, and comprise three different species: A, B, and C. Transmission is primarily by the aerosol route and the virus replicates in the nose.

In some embodiments, the compounds disclosed herein can be used in the treatment of picornavirus infection. In some embodiments, the compounds disclosed herein can be used in the treatment of rhinovirus infection. In some embodiments, the compounds disclosed herein can be used in the treatment of rhinovirus infection wherein the compound binds to free virus, or inhibits a rhinovirus protease. In some cases, the compound can target both (free virus and protease).

The terms, “disease”, “disorder”, and “condition” may be used interchangeably here to refer to norovirus or coronavirus virus mediated medical or pathological condition.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

The term “biological sample”, as used herein, includes, without limitation, cell cultures or extracts thereof; biopsied material obtained from a mammal or extracts thereof; blood, saliva, urine, feces, semen, tears, or other body fluids or extracts thereof.

As used herein, “multiplicity of infection” or “MOI” is the ratio of infectious agents (e.g. phage or virus) to infection targets (e.g. cell). For example, when referring to a group of cells inoculated with infectious virus particles, the multiplicity of infection or MOI is the ratio defined by the number of infectious virus particles deposited in a well divided by the number of target cells present in that well.

As used herein the terms “inhibition of the replication of noroviruses” and “inhibition of the replication of coronaviruses” includes both the reduction in the amount of virus replication (e.g. the reduction by at least 10%) and the complete arrest of virus replication (i.e., 100% reduction in the amount of virus replication). In some embodiments, the replication of norovirus or coronavirus viruses are inhibited by at least 50%, at least 65%, at least 75%, at least 85%, at least 90%, or at least 95%.

Norovirus or coronavirus virus replication can be measured by any suitable method known in the art. For example, norovirus or coronavirus viral titer in a biological sample (e.g. an infected cell culture) or in humans (e.g. lung viral titer in a patient) can be measured. More specifically, for cell based assays, in each case cells are cultured in vitro, virus is added to the culture in the presence or absence of a test agent, and after a suitable length of time a virus-dependent endpoint is evaluated. Such assays are known in the art. A first type of cell assay that can be used in the disclosure depends on death of the infected target cells, a process called cytopathic effect (CPE), where virus infection causes exhaustion of the cell resources and eventual lysis of the cell. In the first type of cell assay, a low fraction of cells in the wells of a microtiter plate are infected (typically 1/10 to 1/1000), the virus is allowed to go through several rounds of replication over 48-72 hours, then the amount of cell death is measured using a decrease in cellular ATP content compared to uninfected controls. A second type of cell assay that can be employed in the disclosure depends on the multiplication of virus-specific RNA molecules in the infected cells, with RNA levels being directly measured using the branched-chain DNA hybridization method (bDNA). In the second type of cell assay, a low number of cells are initially infected in wells of a microtiter plate, the virus is allowed to replicate in the infected cells and spread to additional rounds of cells, then the cells are lysed and viral RNA content is measured. This assay is stopped early, usually after 18-36 hours, while all the target cells are still viable. Viral RNA is quantitated by hybridization to specific oligonucleotide probes fixed to wells of an assay plate, then amplification of the signal by hybridization with additional probes linked to a reporter enzyme.

As used herein a “viral titer (or titer)” is a measure of virus concentration. Titer testing can employ serial dilution to obtain approximate quantitative information from an analytical procedure that inherently only evaluates as positive or negative. The titer corresponds to the highest dilution factor that still yields a positive reading; for example, positive readings in the first 8 serial twofold dilutions translate into a titer of 1:256. To determine the titer, several dilutions will be prepared, such as 10⁻¹, 10⁻², 10⁻³, 10⁻⁸.

As used herein, the terms “treat”, “treatment” and “treating” refer to both therapeutic and prophylactic treatments. For example, therapeutic treatments includes the reduction or mitigation of the progression, severity and/or duration of norovirus or coronavirus mediated conditions, or the amelioration of one or more symptoms (specifically, one or more discernible symptoms) of norovirus or coronavirus mediated conditions, resulting from the administration of one or more therapies (e.g., one or more therapeutic agents such as a compound or composition of the disclosure). In specific embodiments, the therapeutic treatment includes the amelioration of at least one measurable physical parameter of a norovirus or coronavirus mediated condition. In other embodiments the therapeutic treatment includes the inhibition of the progression of a norovirus or coronavirus mediated condition, either physically by, e.g., stabilization of a discernible symptom, physiologically by, e.g., stabilization of a physical parameter, or both. In other embodiments the therapeutic treatment includes the reduction or stabilization of norovirus or coronavirus mediated infections. Antiviral drugs can be used in the community setting to treat people who already have norovirus or coronavirus to reduce the severity of symptoms and reduce the number of days that they are sick.

The term “chemotherapy” refers to the use of medications, e.g. small molecule drugs (rather than “vaccines”) for treating a disorder or disease.

The terms “prophylaxis” or “prophylactic use” and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease. The term “chemoprophylaxis” refers to the use of medications, e.g. small molecule drugs (rather than “vaccines”) for the prevention of a disorder or disease.

As used herein, prophylactic use includes the use in situations in which an outbreak has been detected, to prevent contagion or spread of the infection in places where a lot of people that are at high risk of serious norovirus or coronavirus complications live in close contact with each other (e.g. in a hospital ward, daycare center, prison, nursing home, etc.). It also includes the use among populations who require protection from the norovirus or coronavirus but who either do not get protection after vaccination (e.g. due to weak immune system), or when the vaccine is unavailable to them, or when they cannot get the vaccine because of side effects. It also includes use during the two weeks following vaccination, since during that time the vaccine is still ineffective. Prophylactic use may also include treating a person who is not ill with the norovirus or coronavirus or not considered at high risk for complications, in order to reduce the chances of getting infected with norovirus or coronavirus and passing it on to a high-risk person in close contact with him (for instance, healthcare workers, nursing home workers, etc.).

In some embodiments, the methods of the disclosure are a preventative or “prophylactic” measure to a patient, specifically a human, having a predisposition to complications resulting from infection by a norovirus or coronavirus virus. Prophylactic use includes use in situations in which an “index case” or an “outbreak” has been confirmed, in order to prevent the spread of infection in the rest of the community or population group.

In embodiments, the methods of the disclosure are applied as a “prophylactic” measure to members of a community or population group, specifically humans, in order to prevent the spread of infection.

As used herein, an “effective amount” refers to an amount sufficient to elicit the desired biological response. In the present disclosure the desired biological response is to inhibit the replication of norovirus or coronavirus, to reduce the amount of norovirus or coronavirus or to reduce or ameliorate the severity, duration, progression, or onset of a norovirus or coronavirus virus infection, prevent the advancement of a norovirus or coronavirus infection, prevent the recurrence, development, onset or progression of a symptom associated with a norovirus or coronavirus infection, or enhance or improve the prophylactic or therapeutic effect(s) of another therapy used against norovirus or coronavirus infections. The precise amount of compound administered to a subject will depend on the mode of administration, the type and severity of the infection and on the characteristics of the subject, such as general health, age, sex, body weight and tolerance to drugs. The skilled artisan will be able to determine appropriate dosages depending on these and other factors. When co-administered with other anti-viral agents, e.g., when co-administered with an anti-norovirus or coronavirus medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, a safe and effective amount should be assumed. For example, compounds described herein can be administered to a subject in a dosage range from between approximately 0.01 to 100 mg/kg body weight/day for therapeutic or prophylactic treatment.

Generally, dosage regimens can be selected in accordance with a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe the effective amount of the compounds described herein required to treat, to prevent, inhibit (fully or partially) or arrest the progress of the disease.

Dosages of the compounds for uses described herein can range from between about 0.01 to about 100 mg/kg body weight/day, about 0.01 to about 50 mg/kg body weight/day, about 0.1 to about 50 mg/kg body weight/day, or about 1 to about 25 mg/kg body weight/day. It is understood that the total amount per day can be administered in a single dose or can be administered in multiple dosing, such as twice a day (e.g., every 12 hours), three times a day (e.g., every 8 hours), or four times a day (e.g., every 6 hours).

For therapeutic treatment, the compounds described herein can be administered to a patient within, for example, 48 hours (or within 40 hours, or less than 2 days, or less than 1.5 days, or within 24 hours) of onset of symptoms (e.g., nasal congestion, sore throat, cough, aches, fatigue, headaches, and chills/sweats). The therapeutic treatment can last for any suitable duration, for example, for 5 days, 7 days, 10 days, 14 days, etc. For prophylactic treatment during a community outbreak, the compounds described herein can be administered to a patient within, for example, 2 days of onset of symptoms in the index case, and can be continued for any suitable duration, for example, for 7 days, 10 days, 14 days, 20 days, 28 days, 35 days, 42 days, etc.

### Combination Therapy

The compounds described herein can be used in combination therapy, i.e., in conjunction with other anti-norovirus or anti-coronavirus compounds, or in conjunction with a vaccine. Combination therapy can be particularly advantageous where a patient might be exposed to more than one form of the norovirus or coronavirus virus.

A safe and effective amount can be achieved in the method or pharmaceutical composition of the disclosure employing a compound of Formula I, Table A, Table B, or Table C, or a pharmaceutically acceptable salt thereof alone or in combination with an additional suitable therapeutic agent, for example, an antiviral agent or a vaccine. When “combination therapy” is employed, a safe and effective amount can be achieved using a first amount of a compound of Formula I, Table A, Table B, or Table C, or a pharmaceutically acceptable salt thereof, and a second amount of an additional suitable therapeutic agent (e.g. an antiviral agent or vaccine).

In embodiments, the compound of Formula I, Table A, Table B, or Table C, or a pharmaceutically acceptable salt, and the additional therapeutic agent, are each administered in a safe and effective amount (i.e., each in an amount which would be therapeutically effective if administered alone). In other embodiments, the compound of Formula I, Table A, Table B, or Table C, or a pharmaceutically acceptable salt thereof, and the additional therapeutic agent, are each administered in an amount which alone does not provide a therapeutic effect (a sub-therapeutic dose). In yet other embodiments, the compound of Formula I, Table A, Table B, or Table C, or a pharmaceutically acceptable salt thereof can be administered in a safe and effective amount, while the additional therapeutic agent is administered in a sub-therapeutic dose. In still other embodiments, the compound of Formula I, Table A, Table B, or Table C, a pharmaceutically acceptable salt thereof can be administered in a sub-therapeutic dose, while the additional therapeutic agent, for example, a suitable antiviral therapeutic agent is administered in a safe and effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order.

In embodiments, the present disclosure is directed to methods of combination therapy for inhibiting the virus's replication in biological samples or patients, or for treating or preventing norovirus or coronavirus infections in patients using the compounds or pharmaceutical compositions described herein, e.g., a compound of Formula I, Table A, Table B, or Table C, or a pharmaceutically acceptable salt thereof. Accordingly, pharmaceutical compositions also include those comprising a compound as disclosed herein (e.g., an inhibitor of virus replication) in combination with an anti-viral compound exhibiting anti-Norovirus or coronavirus virus activity.

Methods of use of the compounds and compositions disclosed herein also include combination of chemotherapy with a compound or composition of Formula I, Table A, Table B, or Table C, or a pharmaceutically acceptable salt thereof or with a combination of a compound or composition of this disclosure with another anti-viral agent.

When co-administration involves the separate administration of the first amount of Formula I, Table A, Table B, or Table C, or a pharmaceutically acceptable salt thereof and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of Formula I, Table A, Table B, or Table C, or a pharmaceutically acceptable salt thereof and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound of the disclosure) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-viral agent) to a subject.

It is understood that the method of co-administration of a first amount of a compound of Formula I, Table A, Table B, or Table C, or a pharmaceutically acceptable salt thereof and a second amount of an additional therapeutic agent can result in an enhanced or synergistic therapeutic effect, wherein the combined effect is greater than the additive effect that would result from separate administration of the first amount of the compound of Formula I, Table A, Table B, or Table C, or a pharmaceutically acceptable salt thereof and the second amount of the additional therapeutic agent.

As used herein, the term “synergistic” refers to a combination of a compound disclosed herein and another therapy (e.g., a prophylactic or therapeutic agent), which is more effective than presumed additive effects of the therapies. A synergistic effect of a combination of therapies (e.g., a combination of prophylactic or therapeutic agents) can permit the use of lower dosages of one or more of the therapies and/or less frequent administration of said therapies to a subject. The ability to utilize lower dosages of a therapy (e.g., a prophylactic or therapeutic agent) and/or to administer said therapy less frequently can reduce the toxicity associated with the administration of said therapy to a subject without reducing the efficacy of said therapy in the prevention, management or treatment of a disorder. In addition, a synergistic effect can result in improved efficacy of agents in the prevention, management or treatment of a disorder. Finally, a synergistic effect of a combination of therapies (e.g., a combination of prophylactic or therapeutic agents) may avoid or reduce adverse or unwanted side effects associated with the use of either therapy alone.

When the combination therapy using compounds as disclosed herein is in combination with a virus vaccine, both therapeutic agents can be administered so that the period of time between each administration can be longer (e.g. days, weeks or months).

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S, and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

### Chiral Separations

The compounds described herein can have asymmetric centers and occur as racemates, racemic mixtures, individual diastereomers or enantiomers, with all isomeric forms being included in the present disclosure. Compounds of the present disclosure having a chiral center can exist in and be isolated in optically active and racemic forms. Some compounds can exhibit polymorphism. The present disclosure encompasses racemic, optically-active, polymorphic, or stereoisomeric forms, or mixtures thereof, of a compound of the disclosure, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective compound, then derivatize the compound to form the compounds described herein, or purify the compound themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.

i) physical separation of crystals: a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization: a technique whereby the individual enantiomers are separately crystallized from a solution of the racemate, possible only if the latter is a conglomerate in the solid state;

iii) enzymatic resolutions: a technique whereby partial or complete separation of a racemate by virtue of differing rates of reaction for the enantiomers with an enzyme;

iv) enzymatic asymmetric synthesis: a synthetic technique whereby at least one step of the synthesis uses an enzymatic reaction to obtain an enantiomerically pure or enriched synthetic precursor of the desired enantiomer;

v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;

vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors: a synthetic technique whereby the desired enantiomer is obtained from non-chiral starting materials and where the stereochemical integrity is not or is only minimally compromised over the course of the synthesis;

x) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;

xi) chiral gas chromatography: a technique whereby the racemate is volatilized and enantiomers are separated by virtue of their differing interactions in the gaseous mobile phase with a column containing a fixed non-racemic chiral adsorbent phase;

xii) extraction with chiral solvents: a technique whereby the enantiomers are separated by virtue of preferential dissolution of one enantiomer into a particular chiral solvent;

xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

The present disclosure will be better understood with reference to the following non-limiting examples.

### Compound Synthesis

### Example 1: Synthesis of Compounds C12, C20, and C1

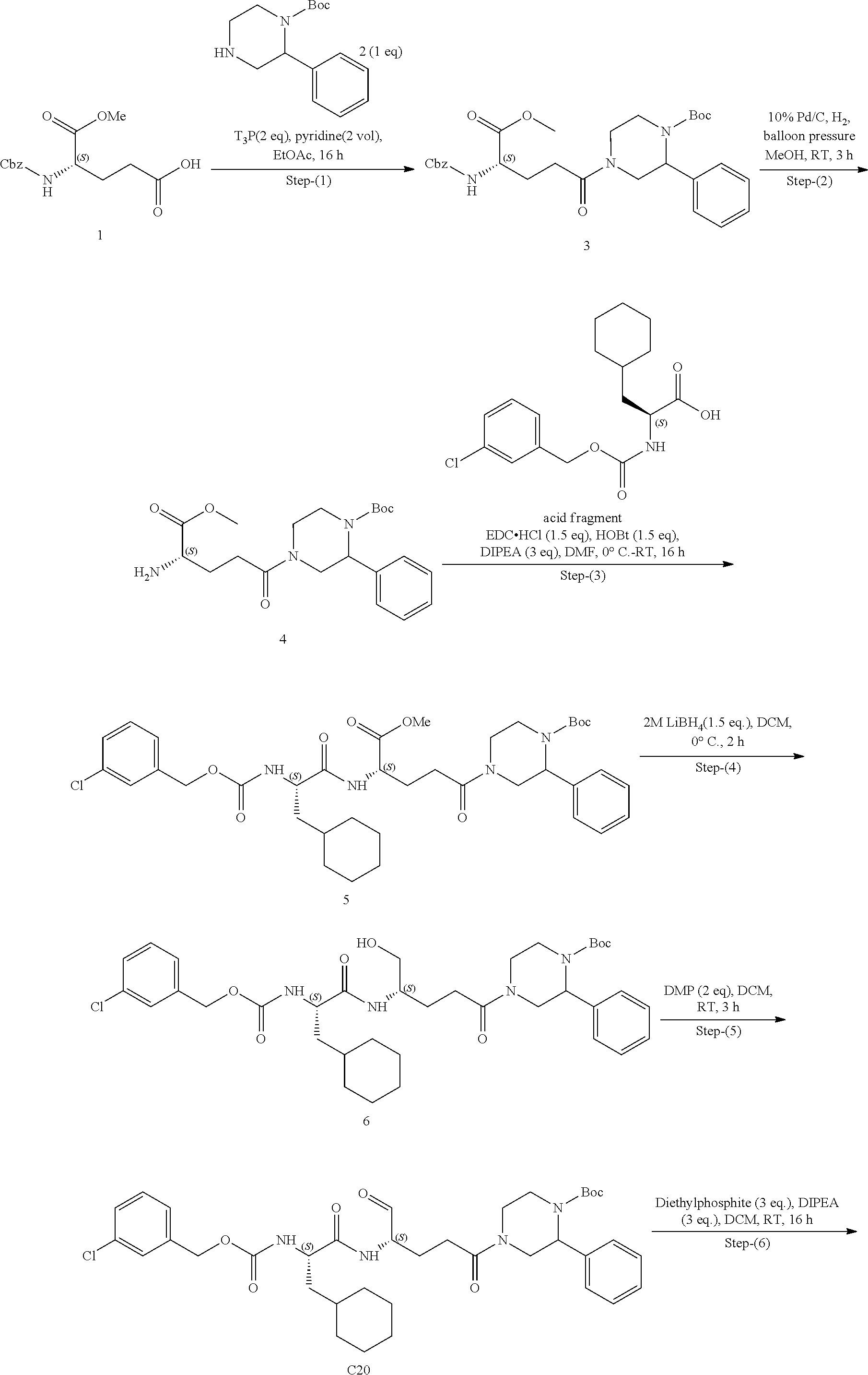

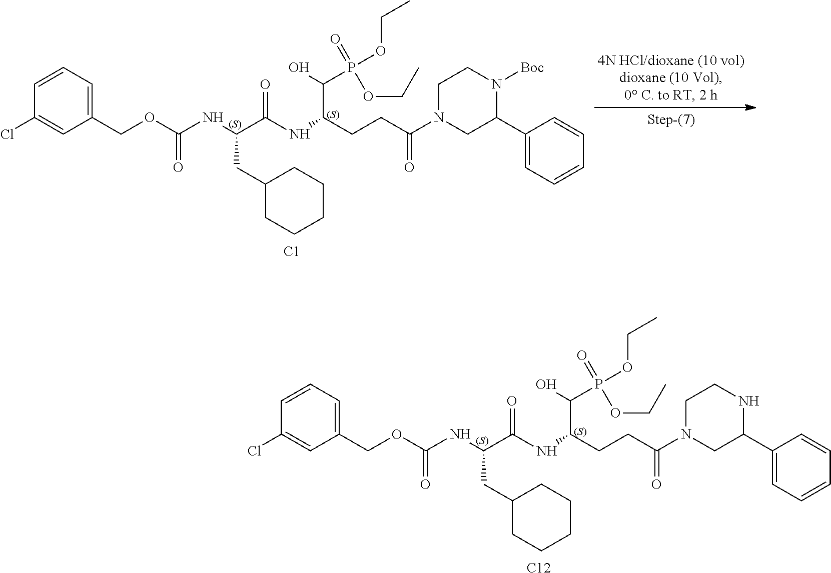

### tert-butyl 4-((S)-4-(((benzyloxy)carbonyl)amino)-5-methoxy-5-oxopentanoyl)-2-phenylpiperazine-1-carboxylate (3)

A mixture of (S)-4-(((benzyloxy)carbonyl)amino)-5-methoxy-5-oxopentanoic acid (1) (1 g, 3.389 mmol), tert-butyl 2-phenylpiperazine-1-carboxylate (2) (888 mg, 3.389 mmol), and pyridine (2 mL, 1 vol) in EtOAc (40 mL) at 0° C. was treated with T₃P (4.31 mL, 50 wt % in EtOAc, 16.129 mmol). The resulting mixture was stirred at RT for 16 h. The progress of the reaction was monitored by TLC and LCMS. After 16 h, reaction mixture was quenched with 1N HCl (20 mL) and added water (50 mL), extracted with ethyl acetate (2×50 mL), the combined organic layer was dried over sodium sulfate, filtered and evaporated under reduced pressure. The crude residue was purified by silica gel column by eluting with 50% ethyl acetate in pet ether to afford Tert-butyl 4-((S)-4-(((benzyloxy)carbonyl)amino)-5-methoxy-5-oxopentanoyl)-2-phenylpiperazine-1-carboxylate (3). TLC system: 50% Ethyl acetate in pet ether, R_{f}: 0.3 LCMS (ESI): m/z 540.40 (M+H)⁺

### tert-butyl 4-((S)-4-amino-5-methoxy-5-oxopentanoyl)-2-phenylpiperazine-1-carboxylate (4)

To a stirred solution of tert-butyl 4-((S)-4-(((benzyloxy)carbonyl)amino)-5-methoxy-5-oxopentanoyl)-2-phenylpiperazine-1-carboxylate (3) (1 g, 1.855 mmol) in MeOH (40 mL) was added 10% Pd/C (500 mg, 50% by wet) at RT and the reaction mixture was stirred at RT for 3 h under H₂ atmosphere (balloon pressure). The progress of the reaction was monitored by TLC and LCMS. After 3 h, the reaction mixture was filtered through celite, washed with MeOH (2×10 mL) and evaporated under reduced pressure to get tert-butyl 4-((S)-4-amino-5-methoxy-5-oxopentanoyl)-2-phenylpiperazine-1-carboxylate (4). TLC system: 5% Methanol in DCM R_{f}: 0.2 LCMS (ESI): m/z 406.35 (M+H)⁺

### tert-butyl 4-((S)-4-((S)-2-((((3-chlorobenzyl)oxy)carbonyl)amino)-3-cyclohexylpropanamido)-5-methoxy-5-oxopentanoyl)-2-phenylpiperazine-1-carboxylate (5)

At 0° C., to a stirred solution of (S)-2-((((3-chlorobenzyl)oxy)carbonyl)amino)-3-cyclohexylpropanoic acid (acid fragment) (1 g, 2.949 mmol) in DMF (20 mL) was added EDC.HCl (845 mg, 4.424 mmol), HOBT (597 mg, 4.424 mmol), DIPEA (1.5 mL, 8.849 mmol) and tert-butyl 4-((S)-4-amino-5-methoxy-5-oxopentanoyl)-2-phenylpiperazine-1-carboxylate (4) (1.38 g, 3.539 mmol) and stirred at room temperature for 16 h. The progress of the reaction was monitored by TLC and LCMS. After 16 h, reaction mixture was quenched with ice water (30 mL), extracted with ethyl acetate (2×30 mL), the combined organic layer was dried over sodium sulfate and evaporated under reduced pressure. The crude residue was purified by silica gel column by eluting with 40% ethyl acetate in pet ether to afford tert-butyl 4-((S)-4-((S)-2-((((3-chlorobenzyl)oxy)carbonyl)amino)-3-cyclohexylpropanamido)-5-methoxy-5-oxopentanoyl)-2-phenylpiperazine-1-carboxylate (5). TLC system: 5% Methanol in DCM R_{f}: 0.4 LCMS (ESI): m/z 727.67 (M+H)⁺

### tert-butyl 4-((S)-4-((S)-2-((((3-chlorobenzyl)oxy)carbonyl)amino)-3-cyclohexylpropanamido)-5-hydroxypentanoyl)-2-phenylpiperazine-1-carboxylate (6)

To a stirred solution of tert-butyl 4-((S)-4-((S)-2-((((3-chlorobenzyl)oxy)carbonyl)amino)-3-cyclohexylpropanamido)-5-methoxy-5-oxopentanoyl)-2-phenylpiperazine-1-carboxylate (5) (900 mg, 1.241 mmol) in DCM (10 mL) was added 2M LiBH₄ in THF (1.24 mL, 2.48 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford the crude tert-butyl 4-((S)-4-((S)-2-((((3-chlorobenzyl)oxy)carbonyl)amino)-3-cyclohexylpropanamido)-5-hydroxypentanoyl)-2-phenylpiperazine-1-carboxylate (6). TLC system: 5% MeOH in DCM R_{f} 0.3 LCMS (ESI): m/z 699.2 (M+H)⁺

### tert-butyl 4-((S)-4-((S)-2-((((3-chlorobenzyl)oxy)carbonyl)amino)-3-cyclohexylpropanamido)-5-oxopentanoyl)-2-phenylpiperazine-1-carboxylate (C20)

To a stirred solution of Tert-butyl 4-((S)-4-((S)-2-((((3-chlorobenzyl)oxy)carbonyl)amino)-3-cyclohexylpropanamido)-5-hydroxypentanoyl)-2-phenylpiperazine-1-carboxylate (6) (150 mg, 0.2148 mmol) in DCM (10 mL) was added Dess-Martin periodinane (273 mg, 0.6446 mmol) at 0° C. and stirred at RT for 3 h. The progress of the reaction was monitored by TLC and LCMS. Reaction mixture was diluted with DCM (50 mL) and washed with sat. NaHCO₃ solution (3×20 mL) followed by sat. Hypo solution (3×20 mL). Organic layer was dried over anhydrous Na₂SO₄, filtered and concentrated to get crude compound Tert-butyl 4-((S)-4-((S)-2-((((3-chlorobenzyl)oxy)carbonyl)amino)-3-cyclohexylpropanamido)-5-oxopentanoyl)-2-phenylpiperazine-1-carboxylate (C20). TLC system: 10% Methanol in DCM R_{f}: 0.3 LCMS (ESI): m/z 697.27 (M+H)⁺

### tert-butyl 4-((4S)-4-((S)-2-((((3-chlorobenzyl)oxy)carbonyl)amino)-3-cyclohexylpropanamido)-5-(diethoxyphosphoryl)-5-hydroxypentanoyl)-2-phenylpiperazine-1-carboxylate (C1)

To a stirred solution of tert-butyl 4-((S)-4-((S)-2-((((3-chlorobenzyl)oxy)carbonyl)amino)-3-cyclohexylpropanamido)-5-oxopentanoyl)-2-phenylpiperazine-1-carboxylate (C20) (200 mg crude, 0.2873 mmol) in DCM (10 mL) was added DIPEA (0.16 mL, 0.8620 mmol) followed by diethylphosphate (0.12 mL, 0.8620 mmol) and the reaction mixture stirred at RT for 16 h. The progress of the reaction was monitored by TLC and LCMS. After 16 h, the reaction mixture quenched with ammonium chloride (15 mL) and extracted with DCM (2×15 mL). Combined organic layer was dried over anhydrous Na₂SO₄, and evaporated to afford crude residue. It was purified prep HPLC to afford tert-butyl 4-((4S)-4-((S)-2-((((3-chlorobenzyl)oxy)carbonyl)amino)-3-cyclohexylpropanamido)-5-(diethoxyphosphoryl)-5-hydroxypentanoyl)-2-phenylpiperazine-1-carboxylate (C1). TLC system: 5% MeOH in DCM R_{f}: 0.4 LCMS (ESI): m/z 835.58 (M+H)⁺

### 3-Chlorobenzyl ((2S)-3-cyclohexyl-1-(((2S)-1-(diethoxyphosphoryl)-1-hydroxy-5-oxo-5-(3-phenylpiperazin-1-yl)pentan-2-yl)amino)-1-oxopropan-2-yl)carbamate (C12)

To a stirred solution of tert-butyl 4-((4S)-4-((S)-2-((((3-chlorobenzyl)oxy)carbonyl)amino)-3-cyclohexylpropanamido)-5-(diethoxyphosphoryl)-5-hydroxypentanoyl)-2-phenylpiperazine-1-carboxylate (C1) (220 mg, 0.2637 mmol) in 1,4-dioxane (2 mL) was added 4N HCl in dioxane (2 mL) with drop wise at 0° C. and the reaction mixture was stirred at RT for 2 h. The progress of the reaction was monitored by TLC and LCMS. After consumption of starting material, the reaction mixture was evaporated under reduced pressure to obtained crude residue. It was purified prep HPLC to afford 3-chlorobenzyl ((2S)-3-cyclohexyl-1-(((2S)-1-(diethoxyphosphoryl)-1-hydroxy-5-oxo-5-(3-phenylpiperazin-1-yl)pentan-2-yl)amino)-1-oxopropan-2-yl)carbamate (C12). TLC system: 10% MeOH in DCM R_{f}: 0.3 LCMS (ESI): m/z 735.53 (M+H)⁺

### Example 2: Synthesis of Compounds C22 and C2

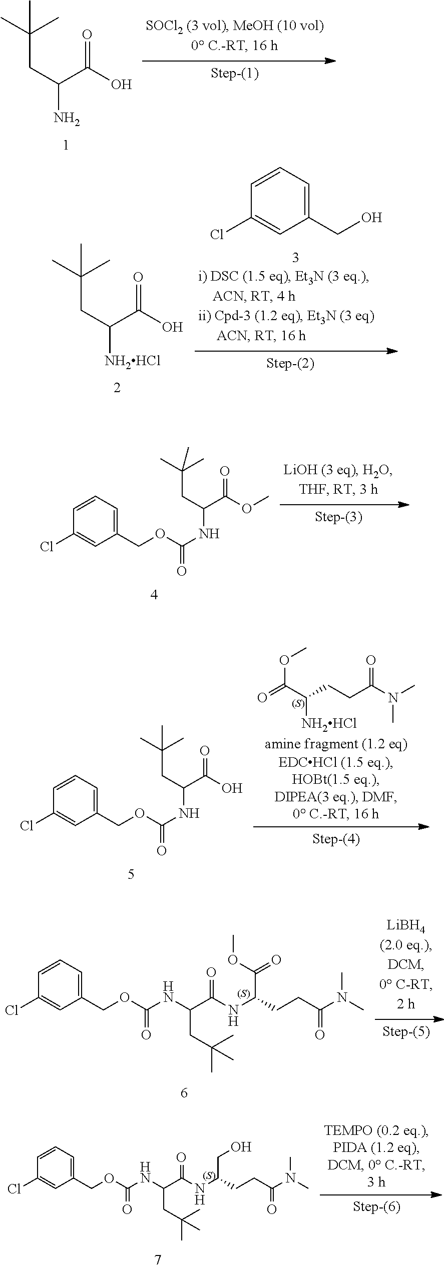

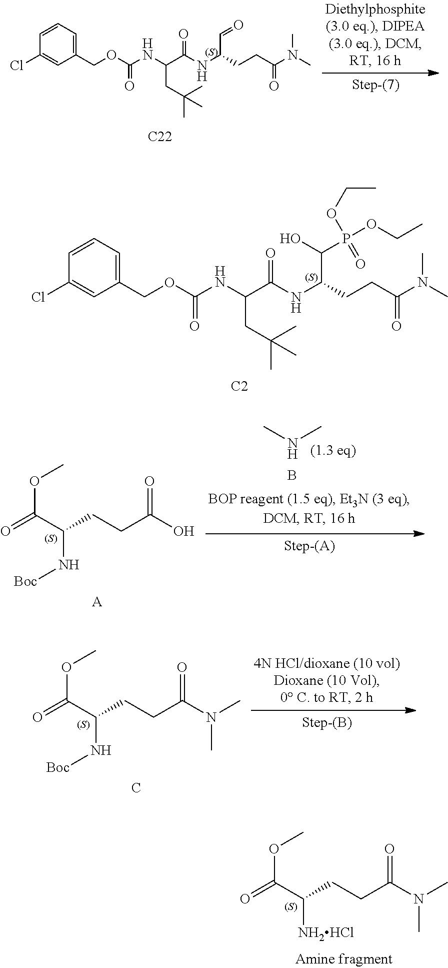

### Methyl N2-(tert-butoxycarbonyl)-N5,N5-dimethyl-L-glutaminate (C)

To a stirred solution of (S)-4-((tert-butoxycarbonyl)amino)-5-methoxy-5-oxopentanoic acid (A) (3 g, 11.4942 mmol) in DCM (30 mL) added TEA (3.2 mL, 22.988 mmol) and were added BOP reagent (7.62 g, 17.241 mmol) and 2M dimethylamine in THF (7.4 mL, 14.942 mmol) (B) at 0° C. and then reaction mixture was stirred at RT for 16 h. The progress of the reaction was monitored by TLC and LCMS. After 16 h, the reaction mixture was diluted with ice water (50 mL), extracted with DCM (2×50 mL). The combined organic layer was dried over sodium sulfate, filtered and evaporated under reduced pressure. The crude residue was purified by combi-flash compound eluted at 40% methanol in DCM to afford methyl N2-(tert-butoxycarbonyl)-N5,N5-dimethyl-L-glutaminate (C). TLC system: 5% MeOH/DCM R_{f}: 0.3 LCMS (ESI): m/z 289.30 [M+H]⁺

### Methyl N5,N5-dimethyl-L-glutaminate hydrochloride (Amine fragment)

To a stirred solution of methyl N2-(tert-butoxycarbonyl)-N5-(3-chlorophenethyl)-N5-methyl-L-glutaminate (C) (1.5 g, 5.208 mmol) in 1,4-dioxane (20 mL) was added 4N HCl in dioxane (15 mL) with drop wise at 0° C. and the reaction mixture was stirred at RT for 2 h. The progress of the reaction was monitored by TLC and LCMS. After consumption of starting material, the reaction mixture was evaporated under reduced pressure to obtained crude compound, the resulting crude triturated with diethyl ether to afford methyl N5,N5-dimethyl-L-glutaminate hydrochloride (amine fragment). TLC system: 5% MeOH/DCM R_{f}: 0.1 LCMS (ESI): m/z 189.17 [M+H]⁺

### methyl 2-amino-4,4-dimethylpentanoate hydrochloride (2)

To a stirred solution of 2-amino-4,4-dimethylpentanoic acid (1) (4 g, 27.586 mmol) in MeOH (40 mL) at RT was added SOCl₂ (12 mL, 3 vol) drop wise at 0° C. and stirred at room temperature for 16 h. The progress of the reaction was monitored by TLC. After consumption of starting material, reaction mixture was evaporated under reduced pressure to obtain crude residue as solid. It was triturated with pet ether and solid was filtered then dried under vacuum to afford methyl 2-amino-4,4-dimethylpentanoate hydrochloride (2). TLC system: 5% MeOH/DCM R_{f}: 0.3

### Methyl 2-((((3-chlorobenzyl)oxy)carbonyl)amino)-4,4-dimethylpentanoate (4)

To a stirred solution of (3-chlorophenyl)methanol (3) (2 g, 14.084 mmol) in ACN (20 mL) was added N,N′ disuccinimidyl carbonate (5.4 g, 21.126 mmol), followed by triethylamine (6 mL, 42.25 mmol) at room temperature and stirred for 16 h. The progress of the reaction was monitored by TLC. The reaction mass was used directly in the subsequent reaction.

In another RB flask, methyl 2-amino-4,4-dimethylpentanoate hydrochloride (2) (3.27 g, 16.901 mmol) was taken in ACN (20 mL), and treated with triethylamine (6 mL, 42.252 mmol). The resulting reaction mixture was stirred for 5 min, then added above prepared reaction mass drop-wise and the reaction mixture stirred at room temperature for 16 h. After 16 h, the reaction mixture was quenched with ice water (15 mL) and extracted with ethyl acetate (2×15 mL), combined organic layers were washed with brine solution (20 mL), dried over sodium sulfate and evaporated under reduced pressure. The crude residue was purified by normal phase chromatography to afford methyl 2-((((3-chlorobenzyl)oxy)carbonyl)amino)-4,4-dimethylpentanoate (4). TLC system: 20% Ethyl acetate in Pet ether Rf: 0.6 LCMS (ESI): m/z: 328.41[M+H]⁻

### 2-((((3-chlorobenzyl)oxy)carbonyl)amino)-4,4-dimethylpentanoic acid (5)

To a stirred solution of methyl 2-((((3-chlorobenzyl)oxy)carbonyl)amino)-4,4-dimethylpentanoate (4) (1.5 g, 4.587 mmol) in THF (20 mL) and water (10 mL), was added lithium hydroxide (330 mg, 13.761 mmol) at RT and stirred at room temperature for 3 h. The progress of the reaction was monitored by TLC and LCMS. After 3 h, the reaction mixture completely distilled under reduced pressure, crude compound acidified with 2N HCL solution up to pH˜4, and extracted with ethyl acetate (2×10 mL), dried over sodium sulfate, concentrated under reduced pressure to afford crude compound 2-((((3-chlorobenzyl)oxy)carbonyl)amino)-4,4-dimethylpentanoic acid (5). TLC system: 20% Ethyl acetate in Pet ether Rf: 0.2 LCMS (ESI): m/z 620.1 (M+H)⁺

### Methyl N2-(2-((((3-chlorobenzyl)oxy)carbonyl)amino)-4,4-dimethylpentanoyl)-N5,N5-dimethyl-L-glutaminate (6)

To a stirred solution of 2-((((3-chlorobenzyl)oxy)carbonyl)amino)-4,4-dimethylpentanoic acid (5) (1.6 g, 5.1118 mmol) DMF (20 mL) added EDC.HCl (1.46 g, 7.667 mmol), HOBT (1.03 g, 7.667 mmol), DIPEA (1.96 mL, 10.6508 mmol) and methyl N5-(3-chlorophenethyl)-N5-methyl-L-glutaminate hydrogen chloride (amine fragment) (2.7 mL, 15.335 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×30 mL). The combined organic layer was dried over sodium sulfate, filtered and evaporated under reduced pressure. The crude residue was purified by combi-flash, compound eluted at 80% Ethyl acetate in pet ether to afford methyl N2-(2-((((3-chlorobenzyl)oxy)carbonyl)amino)-4,4-dimethylpentanoyl)-N5,N5-dimethyl-L-glutaminate (6). TLC system: 5% Methanol in DCM Rf: 0.3 LCMS (ESI): m/z 484.90 [M+H]⁺

### 3-Chlorobenzyl (1-(((S)-5-(dimethylamino)-1-hydroxy-5-oxopentan-2-yl)amino)-4,4-dimethyl-1-oxopentan-2-yl)carbamate (7)

To a stirred solution of methyl N2-(2-((((3-chlorobenzyl)oxy)carbonyl)amino)-4,4-dimethylpentanoyl)-N5,N5-dimethyl-L-glutaminate (6) (600 mg, 1.2422 mmol) in DCM (10 mL) at 0° C. was added 2M LiBH₄ in THF (1.3 mL, 2.484 mmol) and reaction mixture stirred at RT for 2 h. The progress of the reaction was monitored by TLC and LCMS. Then the reaction mixture was quenched with water (30 mL) and extracted with DCM (2×20 mL). Combined organic layer was washed with brine solution, dried over Na₂SO₄ and concentrated to get crude residue. It was purified by silica gel column chromatography to afford 3-chlorobenzyl (1-(((S)-5-(dimethylamino)-1-hydroxy-5-oxopentan-2-yl)amino)-4,4-dimethyl-1-oxopentan-2-yl)carbamate (7). TLC system: 10% Methanol in DCM Rf: 0.1LCMS (ESI): m/z 456.47 (M+H)⁺

### 3-Chlorobenzyl (1-(((S)-5-(dimethylamino)-1,5-dioxopentan-2-yl)amino)-4,4-dimethyl-1-oxopentan-2-yl)carbamate (C22)

To a stirred solution of 3-chlorobenzyl (1-(((S)-5-(dimethylamino)-1-hydroxy-5-oxopentan-2-yl)amino)-4,4-dimethyl-1-oxopentan-2-yl)carbamate (7) (150 mg, 0.329 mmol) in DCM (5 mL) at 0° C. was added PIDA (127 mg, 0.395 mmol) and followed by added TEMPO (10 mg, 0.065 mmol) at 0° C. and stirred at RT for 3 h. The progress of the reaction was monitored by TLC and LCMS. Reaction mixture was diluted with DCM (10 mL) and washed with sat. Hypo solution (3×20 mL) followed by saturated NaHCO₃ solution (3×20 mL). Organic layer was dried over anhydrous Na₂SO₄, filtered and concentrated to get crude residue. It was purified by prep HPLC to afford 3-chlorobenzyl (1-(((S)-5-(dimethylamino)-1,5-dioxopentan-2-yl)amino)-4,4-dimethyl-1-oxopentan-2-yl)carbamate (C22). TLC system: 10% MeOH in DCM Rf: 0.4 LCMS (ESI): m/z 454.25 (M+H)⁺

### 3-Chlorobenzyl (1-(((2S)-1-(diethoxyphosphoryl)-5-(dimethylamino)-1-hydroxy-5-oxopentan-2-yl)amino)-4,4-dimethyl-1-oxopentan-2-yl)carbamate (C2)

To a stirred solution of 3-chlorobenzyl (1-(((S)-5-(dimethylamino)-1,5-dioxopentan-2-yl)amino)-4,4-dimethyl-1-oxopentan-2-yl)carbamate (C22) (200 mg, 0.4415 mmol) in DCM (10 mL) added DIPEA (0.2 mL, 1.324 mmol) followed by added diethyl phosphite (0.2 mL, 1.324 mmol) and the reaction mixture stirred at RT for 16 h. The progress of the reaction was monitored by TLC and LCMS. After 16 h, the reaction mixture quenched with ammonium chloride (15 mL) and extracted with DCM (2×20 mL). Combined organic layer was dried over anhydrous Na₂SO₄, and evaporated to afford crude residue. It was purified prep HPLC to afford 3-chlorobenzyl (1-(((2S)-1-(diethoxyphosphoryl)-5-(dimethylamino)-1-hydroxy-5-oxopentan-2-yl)amino)-4,4-dimethyl-1-oxopentan-2-yl)carbamate (C2). TLC system: 5% Methanol in DCM Rf: 0.3 LCMS (ESI): m/z 592.51 (M+H)⁺

### Example 3: Synthesis of Compounds C16 and C6

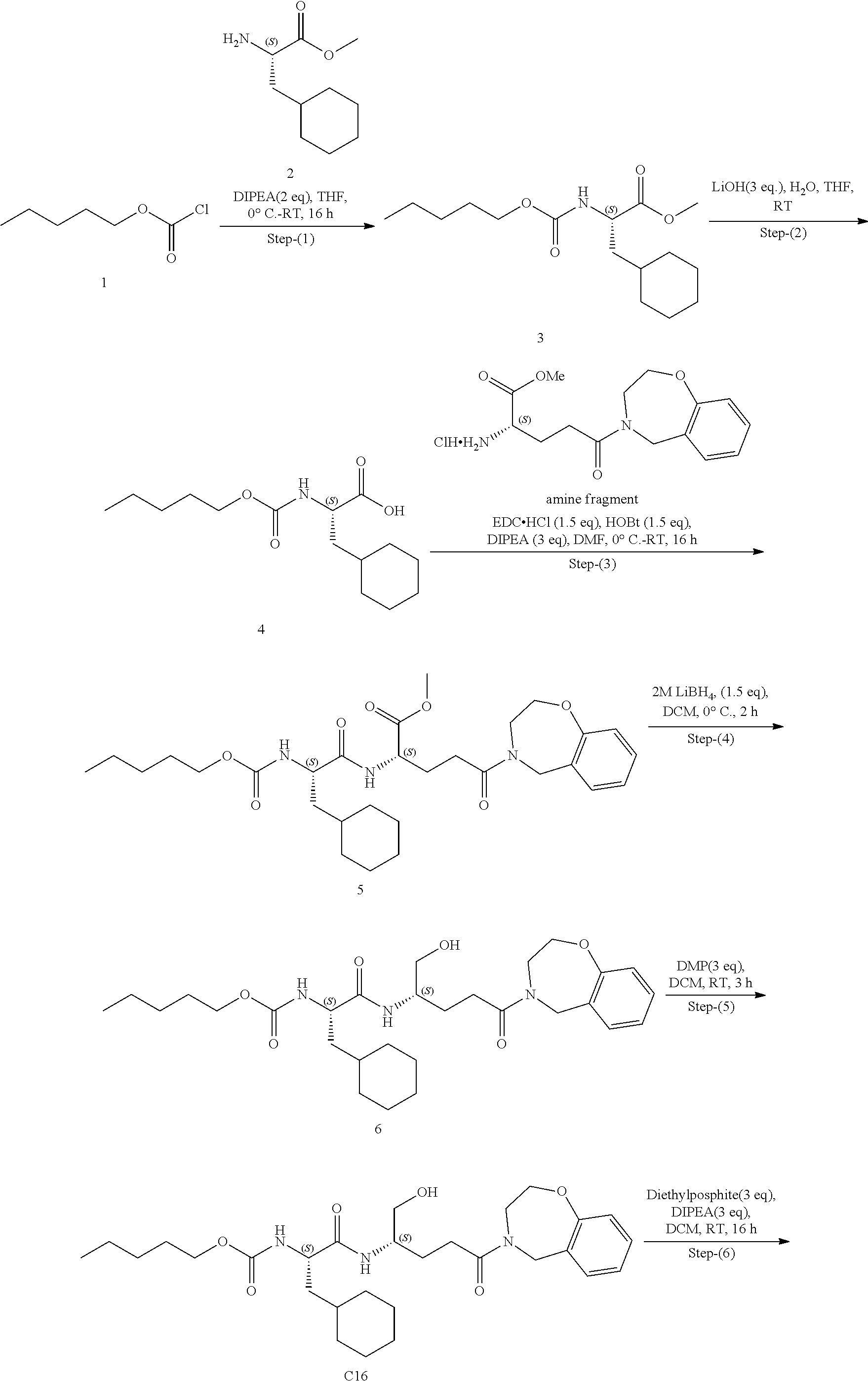

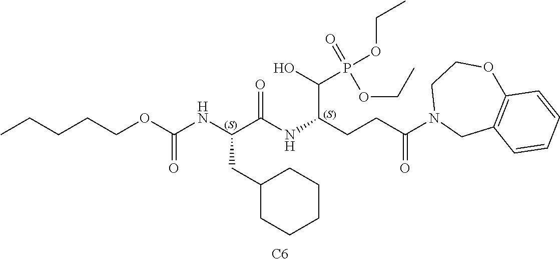

### Methyl (S)-3-cyclohexyl-2-(((pentyloxy)carbonyl)amino)propanoate (3)

To a stirred solution of (S)-2-amino-3-cyclohexylpropanoate hydrochloride (2) (3 g, 13.531 mmol), in THF (20 mL) and DIPEA (7 mL, 40.59 mmol) at 0° C. was added pentyl carbonochloridate (1) (2.34 mL, 16.2 mmol). The resulting mixture was stirred at RT for 2 h. The progress of the reaction was monitored by TLC and LCMS. After 2 h, reaction mixture was quenched with water (50 mL) and extracted with ethyl acetate (2×80 mL), the combined organic layer was dried over sodium sulfate, filtered and evaporated under reduced pressure. The crude residue was purified by silica gel column by eluting with 50% ethyl acetate in pet ether to afford methyl (S)-3-cyclohexyl-2-(((pentyloxy)carbonyl)amino)propanoate. TLC system: 30% Ethyl acetate in pet ether R_{f}: 0.55 LCMS (ESI): m/z 330.2 (M+NH)⁺

### (S)-3-Cyclohexyl-2-(((pentyloxy)carbonyl)amino)propanoic acid (4)

To a stirred solution of methyl (S)-3-cyclohexyl-2-(((pentyloxy)carbonyl)amino)propanoate (3) (2.5 g, 8.3 mmol) in THF (20 mL) and water (5 mL), was added lithium hydroxide (600 mg, 25 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH˜4, and extracted with dichloromethane (2×30 mL), dried over sodium sulfate, concentrated under reduced pressure to afford (S)-3-Cyclohexyl-2-(((pentyloxy)carbonyl)amino)propanoic acid (4). TLC system: 5% Methanol in DCM R_{f}: 0.2

### Methyl (S)-2-((S)-3-cyclohexyl-2-(((pentyloxy)carbonyl)amino)propanamido)-5-(2,3-dihydrobenzo[f][1,4]oxazepin-4(5H)-yl)-5-oxopentanoate (5)

To a stirred solution of (S)-3-cyclohexyl-2-(((pentyloxy)carbonyl)amino)propanoic acid (4) (1 g, 3.5 mmol) in DMF (20 mL) at 0° C. was added EDC.HCl (1 g, 5.2 mmol), HOBT (700 mg, 5.23 mmol), DIPEA (1.7 mL, 10.46 mmol) and methyl (S)-2-amino-5-(2,3-dihydrobenzo[f][1,4]oxazepin-4(5H)-yl)-5-oxopentanoate hydrochloride (Amine fragment) (1.38 g, 4.2 mmol) simultaneously and stirred at room temperature for 16 h. The progress of the reaction was monitored by TLC and LCMS. After 16 h, reaction mixture was quenched with ice water (30 mL), extracted with ethyl acetate (2×60 mL), the combined organic layer was dried over sodium sulfate and evaporated under reduced pressure. The crude residue was purified by silica gel column by eluting with 40% ethyl acetate in pet ether to afford methyl (S)-2-((S)-3-cyclohexyl-2-(((pentyloxy)carbonyl)amino)propanamido)-5-(2,3-dihydrobenzo[f][1,4]oxazepin-4(5H)-yl)-5-oxopentanoate (5). TLC system: 5% Methanol in DCM R_{f}: 0.6 LCMS (ESI): m/z 560.63 (M+H)⁺

### Pentyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6)

To a stirred solution of methyl (S)-2-((S)-3-cyclohexyl-2-(((pentyloxy)carbonyl)amino)propanamido)-5-(2,3-dihydrobenzo[f][1,4]oxazepin-4(5H)-yl)-5-oxopentanoate (5) (900 mg, 1.6 mmol) in DCM (10 mL) was added 2M LiBH₄ in THF (1.2 mL, 1.53 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford the crude pentyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6). TLC system: 5% MeOH in DCM R_{f} 0.3 LCMS (ESI): m/z 532.5 (M+H)⁺

### Pentyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (C16)

To a stirred solution of pentyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6) (200 mg, 0.376 mmol) in DCM (5 mL) was added Dess-Martin periodinane (479 mg, 1.13 mmol) at 0° C. and stirred at RT for 3 h. The progress of the reaction was monitored by TLC and LCMS. Reaction mixture was diluted with DCM (50 mL) and washed with sat. NaHCO₃ solution (3×20 mL) followed by sat. Hypo solution (3×20 mL). Organic layer was dried over anhydrous Na₂SO₄, filtered and concentrated to get crude compound pentyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (C16). TLC system: 5% Methanol in DCM R_{f}: 0.4 LCMS (ESI): m/z 530.56 (M+H)⁺

### Pentyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (C6)

To a stirred solution of pentyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (C16) (248 mg, 0.47 mmol) in DCM (5 mL) was added DIPEA (0.24 mL, 1.41 mmol) followed by diethylphosphate (0.19 mL, 1.41 mmol) and the reaction mixture stirred at RT for 16 h. The progress of the reaction was monitored by TLC and LCMS. After 16 h, the reaction mixture quenched with ammonium chloride (15 mL) and extracted with DCM (2×15 mL). Combined organic layer was dried over anhydrous Na₂SO₄, and evaporated to afford crude residue. It was purified by prep HPLC to Pentyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (C6). TLC system: 5% MeOH in DCM R_{f}: 0.45LCMS (ESI): m/z 668.68 (M+H)⁺

### Example 4: Synthesis of Compounds C26 and C7

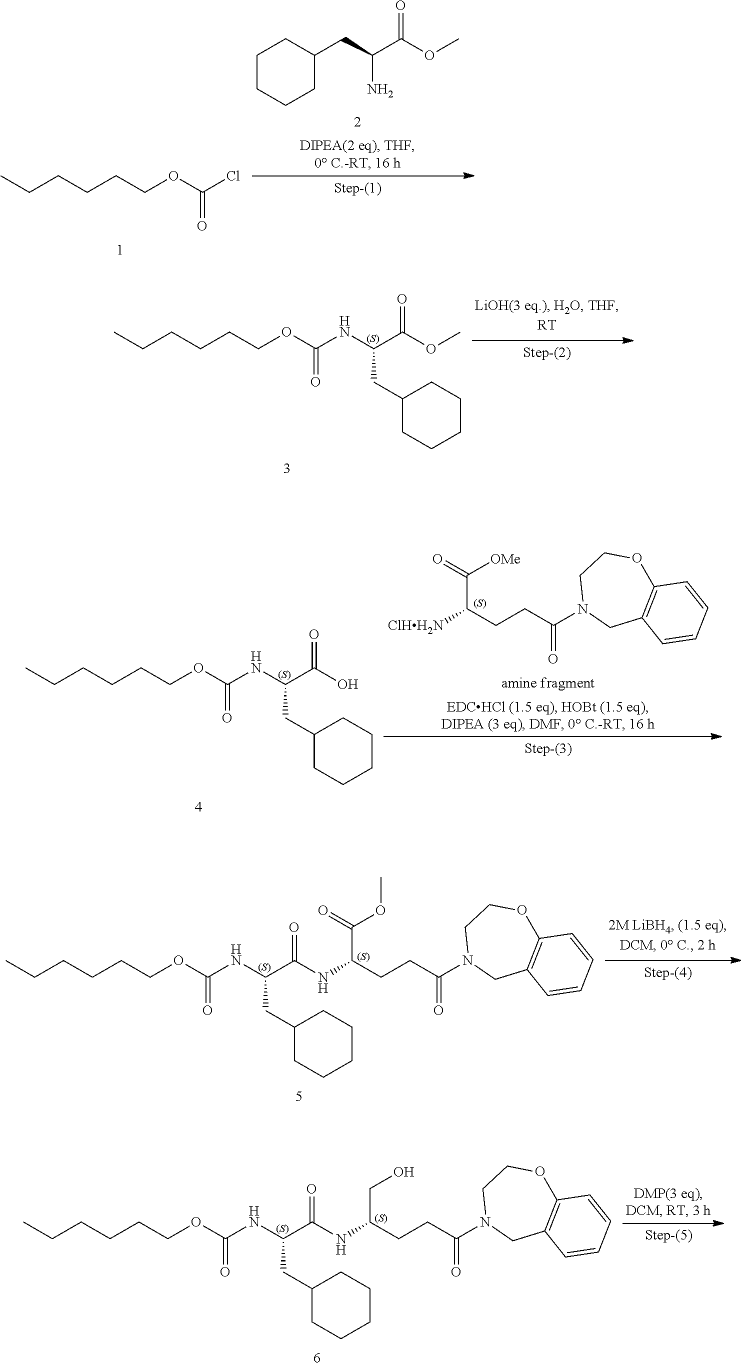

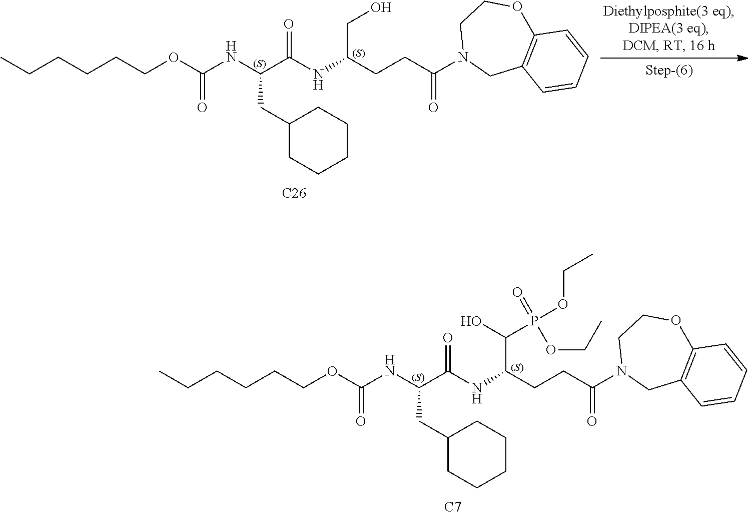

### Methyl (S)-3-cyclohexyl-2-(((hexyloxy)carbonyl)amino)propanoate (3)

To a stirred solution of (S)-2-amino-3-cyclohexylpropanoate hydrochloride (2) (4.8 g, 2.1 mmol), in THF (20 mL) and DIPEA (9.7 mL, 5.4 mmol) at 0° C. was added hexyl carbonochloridate (1) (3 g, 1.8 mmol). The resulting mixture was stirred at RT for 2 h. The progress of the reaction was monitored by TLC and LCMS. After 2 h, reaction mixture was quenched with water (50 mL) and extracted with ethyl acetate (2×80 mL), the combined organic layer was dried over sodium sulfate, filtered and evaporated under reduced pressure. The crude residue was purified by silica gel column by eluting with 50% ethyl acetate in pet ether to afford methyl (S)-3-cyclohexyl-2-(((hexyloxy)carbonyl)amino)propanoate (3). TLC system: 5% MeOH in DCM R_{f}: 0.55 LCMS (ESI): m/z 314.42 (M+H)⁺

### (S)-3-Cyclohexyl-2-(((hexyloxy)carbonyl)amino)propanoic acid (4)

To a stirred solution of methyl (S)-3-cyclohexyl-2-(((hexyloxy)carbonyl)amino)propanoate (3) (2 g, 6.36 mmol) in THF (20 mL) and water (5 mL), was added lithium hydroxide (450 mg, 19 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH˜4, and extracted with dichloromethane (2×30 mL), dried over sodium sulfate, concentrated under reduced pressure to afford (S)-3-Cyclohexyl-2-(((hexyloxy)carbonyl)amino)propanoic acid (4). TLC system: 5% Methanol in DCM R_{f}: 0.2 LCMS (ESI): m/z 300.2 (M+H)⁺

### tert-Butyl 1-((S)-4-((S)-2-((((3-chlorobenzyl)oxy)carbonyl)amino)-3-cyclohexylpropanamido)-5-methoxy-5-oxopentanoyl)-1,2,3,5-tetrahydro-4H-benzo[e][1,4]diazepine-4-carboxylate (5)

At 0° C., to a stirred solution of (S)-3-cyclohexyl-2-(((hexyloxy)carbonyl)amino)propanoic acid (1.2 g, 4 mmol) in DMF (20 mL) was added EDC.HCl (1.14 g, 6 mmol), HOBT (834 mg, 6 mmol), DIPEA (2 mL, 12 mmol) and methyl (S)-2-amino-5-(2,3-dihydrobenzo[f][1,4]oxazepin-4(5H)-yl)-5-oxopentanoate hydrochloride (Amine fragment) (1.57 g, 4.8 mmol) and stirred at room temperature for 16 h. The progress of the reaction was monitored by TLC and LCMS. After 16 h, reaction mixture was quenched with ice water (500 mL), extracted with ethyl acetate (2×50 mL), the combined organic layer was dried over sodium sulfate and evaporated under reduced pressure. The crude residue was purified by silica gel column by eluting with 40% ethyl acetate in pet ether to afford tert-Butyl 1-((S)-4-((S)-2-((((3-chlorobenzyl)oxy)carbonyl)amino)-3-cyclohexylpropanamido)-5-methoxy-5-oxopentanoyl)-1,2,3,5-tetrahydro-4H-benzo[e][1,4]diazepine-4-carboxylate (5). TLC system: 5% Methanol in DCM R_{f}: 0.4 LCMS (ESI): m/z 574.53 (M+H)⁺

### Hexyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6)

To a stirred solution of methyl (S)-2-((S)-3-cyclohexyl-2-(((hexyloxy)carbonyl)amino)propanamido)-5-(2,3-dihydrobenzo[f][1,4]oxazepin-4(5H)-yl)-5-oxopentanoate (5) (960 mg, 1.67 mmol) in DCM (10 mL) was added 2M LiBH₄ in THF (1.25 mL, 1.5 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford the crude hexyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6). TLC system: 5% MeOH in DCM R_{f} 0.3 LCMS (ESI): m/z 546.51 (M+H)⁺

### Hexyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (C26)

To a stirred solution of hexyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6) (250 mg, 0.45 mmol) in DCM (5 mL) was added Dess-Martin periodinane (583 mg, 1.37 mmol) at 0° C. and stirred at RT for 3 h. The progress of the reaction was monitored by TLC and LCMS. Reaction mixture was diluted with DCM (50 mL) and washed with sat. NaHCO₃ solution (3×20 mL) followed by sat. Hypo solution (3×20 mL). Organic layer was dried over anhydrous Na₂SO₄, filtered and concentrated to get crude hexyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (C26). TLC system: 5% Methanol in DCM R_{f}: 0.5 LCMS (ESI): m/z 544.55 (M+H)⁺

### Hexyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (C7)

To a stirred solution of hexyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (248 mg, 0.45 mmol) in DCM (5 mL) was added DIPEA (0.23 mL, 1.37 mmol) followed by diethylphosphate (0.18 mL, 1.37 mmol) and the reaction mixture stirred at RT for 16 h. The progress of the reaction was monitored by TLC and LCMS. After 16 h, the reaction mixture quenched with ammonium chloride (15 mL) and extracted with DCM (2×15 mL). Combined organic layer was dried over anhydrous Na₂SO₄, and evaporated to afford crude residue. It was purified prep HPLC to afford Hexyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (C7). TLC system: 5% MeOH in DCM R_{f}: 0.5 LCMS (ESI): m/z 682.6 (M+H)⁺

### Example 5: Synthesis of Compound C15 and C9

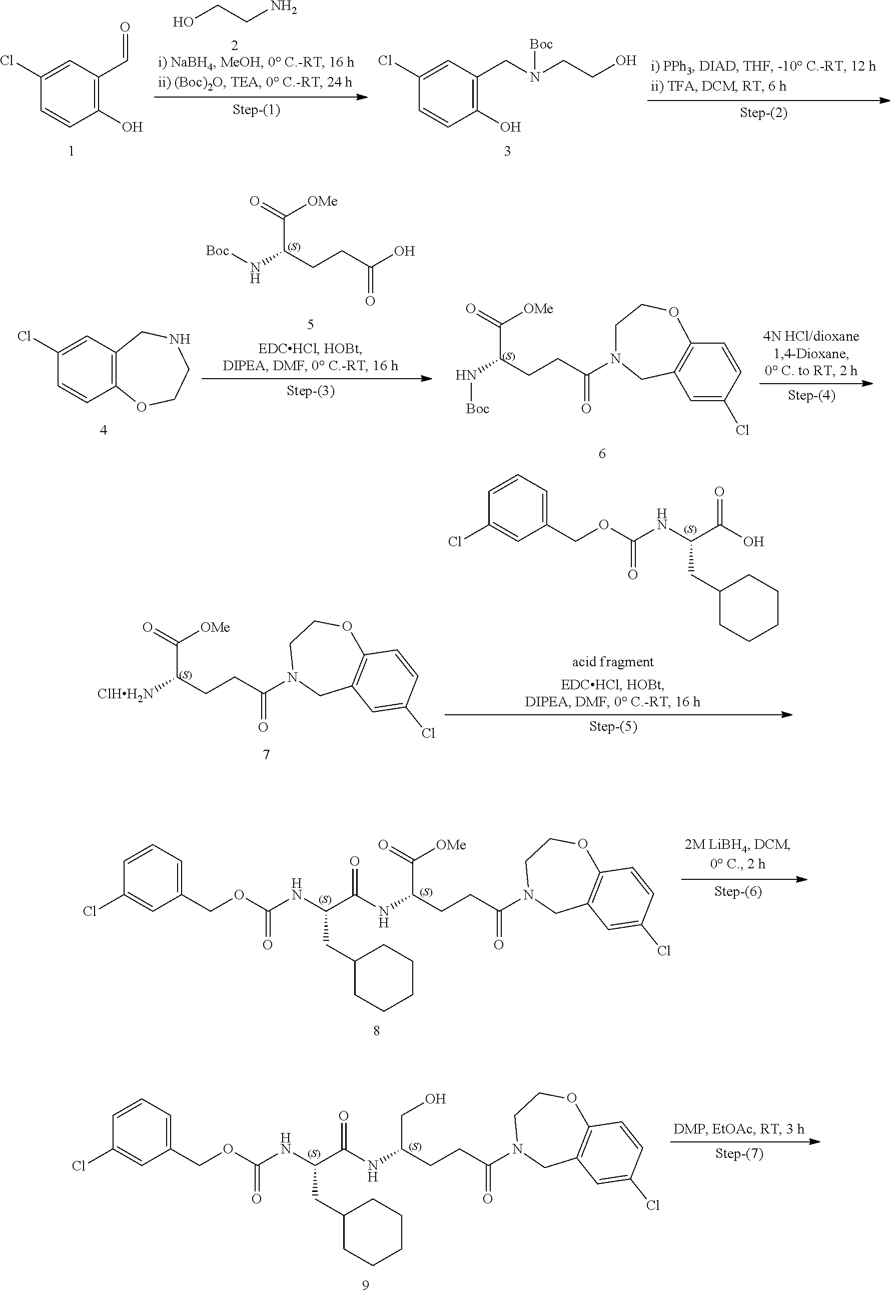

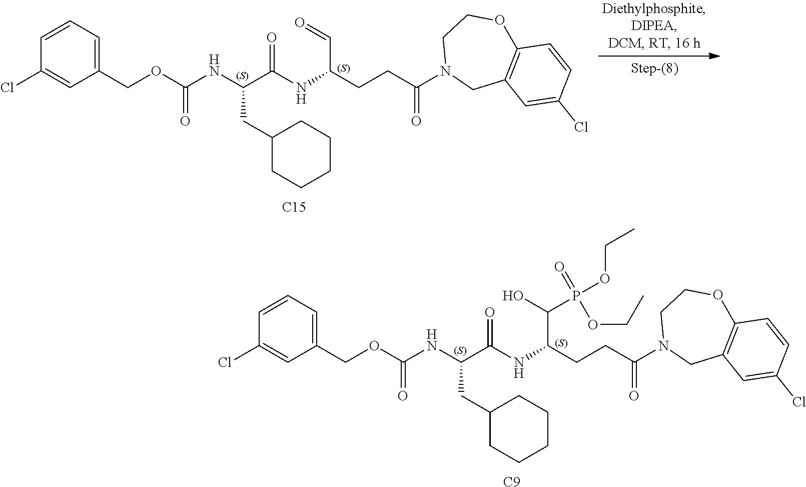

### Tert-butyl (5-chloro-2-hydroxybenzyl) (2-hydroxyethyl) carbamate (3)

To a stirred solution of 5-chloro-2-hydroxybenzaldehyde (1) (5 g, 32.05 mmol) in methanol (50 mL) was added 2-aminoethan-1-ol (2) (1.95 mL, 32.05 mmol) at RT and stirred for 6 h then NaBH₄ (605 mg, 16.02 mmol) was added at 0° C. and stirred for 6 h. Reaction mixture was cooled to 0° C. and added triethylamine (2.8 mL, 19.93 mmol), (Boc)₂O (3.98 g, 18.27 mmol) and allowed to RT for 16 h. The progress of the reaction was monitored by TLC and LCMS. The reaction mixture was evaporated under reduced pressure and acidified up to pH˜4 with 2N HCl, solids were filtered and washed with water (100 mL), dried under vacuum to afford tert-butyl (5-chloro-2-hydroxybenzyl)(2-hydroxyethyl)carbamate (3). TLC system: 50% Ethyl acetate in Pet ether Rf: 0.4 LCMS (ESI): m/z 300.35 [M−H]

### 7-Chloro-2, 3, 4, 5-tetrahydrobenzo[f][1, 4]oxazepine (4)

To a stirred solution of DIAD (3.25 g, 16.12 mmol), triphenyl phosphine (4.22 g, 13.12 mmol) in THF (50 mL) was added slowly tert-butyl (5-chloro-2-hydroxybenzyl) (2-hydroxyethyl) carbamate (3) (5 g, 16.61 mmol) in THF (20 mL) at −10° C. and allowed to RT for 16 h. The progress of the reaction was monitored by TLC. The reaction mixture was evaporated under reduced pressure and dissolved in dichloromethane (50 mL) and added TFA (17 mL) at 0° C. and stirred at RT for 5 h. The progress of the reaction was monitored by TLC, Reaction mixture was basified pH˜12 with 10% NaOH and extracted with DCM (2×50 mL), dried over sodium sulfate, concentrated under reduced pressure to afford 7-chloro-2,3,4,5-tetrahydrobenzo[f][1,4] oxazepine (4). TLC system: 100% Ethyl acetate Rf: 0.25 LCMS (ESI): m/z=184.33 [M+H]⁺

### Methyl (S)-2-((tert-butoxycarbonyl) amino)-5-(7-chloro-2,3-dihydrobenzo[f][1,4] oxazepin-4(5H)-yl)-5-oxopentanoate (5)

To a stirred solution of methyl (S)-4-((tert-butoxycarbonyl)amino)-5-methoxy-5-oxopentanoic acid (5) (1.5 g, 57.47 mmol) in DMF (20 mL), added EDC.HCl (1.64 g, 86.20 mmol), HOBT (1.16 g, 86.20 mmol), DIPEA (3.17 mL, 172.3 mmol) and 7-chloro-2,3,4,5-tetrahydrobenzo[f][1,4]oxazepine (4) (2.1 g, 68.96 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×50 mL). The combined organic layer was dried over sodium sulfate, filtered and evaporated under reduced pressure. The crude residue was purified by combi-flash, compound eluted at 50% Ethyl acetate in pet ether to afford methyl (S)-2-((tert-butoxycarbonyl)amino)-5-(7-chloro-2,3-dihydrobenzo[f][1,4]oxazepin-4(5H)-yl)-5-oxopentanoate (6). TLC system: 70% Ethyl acetate in Pet ether R_{f}: 0.5 LCMS (ESI): m/z 427.36 [M+H]⁺

### Methyl (S)-2-amino-5-(7-chloro-2,3-dihydrobenzo[f][1, 4] oxazepin-4(5H)-yl)-5-oxopentanoate hydrochloride (7)

To a stirred solution of methyl (S)-2-((tert-butoxycarbonyl)amino)-5-(7-chloro-2,3-dihydrobenzo[f][1,4]oxazepin-4(5H)-yl)-5-oxopentanoate (6) (2 g, 4.69 mmol) in 1,4-dioxane (20 mL) was added 4N HCl in dioxane (20 mL) with drop wise at 0° C. and the reaction mixture was stirred at RT for 2 h. The progress of the reaction was monitored by TLC and LCMS. After consumption of starting material, the reaction mixture was evaporated under reduced pressure to obtained crude compound, the resulting crude triturated with diethyl ether to afford methyl (S)-2-amino-5-(7-chloro-2,3-dihydrobenzo[f][1,4]oxazepin-4(5H)-yl)-5-oxopentanoate hydrochloride (7). TLC system: 5% Methanol in DCM Rf: 0.1 LCMS (ESI): m/z 327.25 [M+H]⁺

### Methyl (S)-5-(7-chloro-2,3-dihydrobenzo[f][1,4]oxazepin-4(5H)-yl)-2-((S)-2-((((3-chlorobenzyl)oxy)carbonyl)amino)-3-cyclohexylpropanamido)-5-oxopentanoate (7)

To a stirred solution of (S)-2-((((3-chlorobenzyl)oxy)carbonyl)amino)-3-cyclohexylpropanoic acid (acid fragment) (0.7 g, 2.064 mmol) DMF (10 mL) added EDC.HCl (0.59 g, 3.097 mmol), HOBT (0.418 g, 3.097 mmol), DIPEA (0.5 mL, 6.19 mmol) and methyl (S)-2-amino-5-(7-chloro-2,3-dihydrobenzo[f][1,4]oxazepin-4(5H)-yl)-5-oxopentanoate hydrochloride (7) (0.896 g, 2.477 mmol) at 0° C. simultaneously and stirred at room temperature for 16 h. The progress of the reaction was monitored by TLC and LCMS. After 16 h, reaction mixture was diluted with ice water (25 mL), extracted with ethyl acetate (2×30 mL). The combined organic layer was dried over sodium sulfate, filtered and evaporated under reduced pressure. The crude residue was purified by combi-flash, compound eluted at 45% ethyl acetate in pet ether to afford methyl (S)-5-(7-chloro-2,3-dihydrobenzo[f][1,4]oxazepin-4(5H)-yl)-2-((S)-2-((((3-chlorobenzyl)oxy)carbonyl)amino)-3-cyclohexylpropanamido)-5-oxopentanoate (8). TLC system: 50% Ethyl acetate in Pet ether Rf: 0.5 LCMS (ESI): m/z 647.22 [M+H]⁺

### 3-Chlorobenzyl ((S)-1-(((S)-5-(7-chloro-2,3-dihydrobenzo[f][1,4]oxazepin-4(5H)-yl)-1-hydroxy-5-oxopentan-2-yl)amino)-3-cyclohexyl-1-oxopropan-2-yl)carbamate (8)

To a stirred solution of methyl (S)-5-(7-chloro-2,3-dihydrobenzo[f][1,4]oxazepin-4(5H)-yl)-2-((S)-2-((((3-chlorobenzyl)oxy)carbonyl)amino)-3-cyclohexylpropanamido)-5-oxopentanoate (8) (0.3 g, 0.463 mmol) in DCM (5 mL) was added 2M LiBH₄ in THF (0.7 mL, 0.46 mmol) at 0° C. and the reaction mixture stirred for 2 h at 0° C. The progress of the reaction was monitored by TLC and LCMS. Then reaction mixture was quenched with Aq. NH₄Cl (10 mL) and extracted with ethyl acetate (2×15 mL). Organic layer was washed with brine solution (20 mL), dried over Na₂SO₄ and concentrated to get compound to afford 3-chlorobenzyl ((S)-1-(((S)-5-(7-chloro-2,3-dihydrobenzo[f][1,4]oxazepin-4(5H)-yl)-1-hydroxy-5-oxopentan-2-yl)amino)-3-cyclohexyl-1-oxopropan-2-yl)carbamate (9). TLC system: 5% Methanol in DCM Rf: 0.3 LCMS (ESI): m/z 620.26 (M+H)⁺

### 3-Chlorobenzyl ((S)-1-(((S)-5-(7-chloro-2,3-dihydrobenzo[f][1,4]oxazepin-4(5H)-yl)-1,5-dioxopentan-2-yl)amino)-3-cyclohexyl-1-oxopropan-2-yl)carbamate (C15)

To a stirred solution of 3-chlorobenzyl ((S)-1-(((S)-5-(7-chloro-2,3-dihydrobenzo[f][1,4]oxazepin-4(5H)-yl)-1-hydroxy-5-oxopentan-2-yl)amino)-3-cyclohexyl-1-oxopropan-2-yl)carbamate (9) (200 mg, 0.33 mmol) was dissolved in ethyl acetate (10 mL) was added Dess-Martin periodinane (426 mg, 1.005 mmol) at 0° C. and stirred at RT for 3 h. Reaction mixture was diluted with ethyl acetate (20 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by combi-flash chromatography by eluting 3% methanol in dichloromethane to afford 3-chlorobenzyl ((S)-1-(((S)-5-(7-chloro-2,3-dihydrobenzo[f][1,4]oxazepin-4(5H)-yl)-1,5-dioxopentan-2-yl)amino)-3-cyclohexyl-1-oxopropan-2-yl)carbamate (C15). TLC system: 5% Methanol in DCM Rf: 0.5 LCMS (ESI): m/z 618.33 (M+H)⁺

### 3-Chlorobenzyl ((2S)-1-(((2S)-5-(7-chloro-2,3-dihydrobenzo[f][1,4]oxazepin-4(5H)-yl)-1-(diethoxyphosphoryl)-1-hydroxy-5-oxopentan-2-yl)amino)-3-cyclohexyl-1-oxopropan-2-yl)carbamate (C9)

To a stirred solution of 3-chlorobenzyl ((S)-1-(((S)-5-(7-chloro-2,3-dihydrobenzo[f][1,4]oxazepin-4 (5H)-yl)-1,5-dioxopentan-2-yl) amino)-3-cyclohexyl-1-oxopropan-2-yl) carbamate (C15) (250 mg crude, 0.405 mmol) in DCM (10 mL) added DIPEA (0.2 mL, 1.215 mmol) followed by diethyl phosphite (0.12 mL, 1.215 mmol) and the reaction mixture stirred at RT for 16 h. The progress of the reaction was monitored by TLC and LCMS. After 16 h, the reaction mixture quenched with sat. ammonium chloride (20 mL) and extracted with DCM (2×20 mL). Combined organic layer was dried over anhydrous Na₂SO₄, and evaporated to afford crude residue. It was purified by prep HPLC purification to afford 3-chlorobenzyl ((2S)-1-(((2S)-5-(7-chloro-2,3-dihydrobenzo[f][1,4]oxazepin-4 (5H)-yl)-1-(diethoxyphosphoryl)-1-hydroxy-5-oxopentan-2-yl)amino)-3-cyclohexyl-1-oxopropan-2-yl)carbamate (C9). TLC system: 5% Methanol in DCM R_{f}: 0.3 LCMS (ESI): m/z 756.10 [M+H]⁺

### Example 6: Synthesis of Compounds C29 and C11

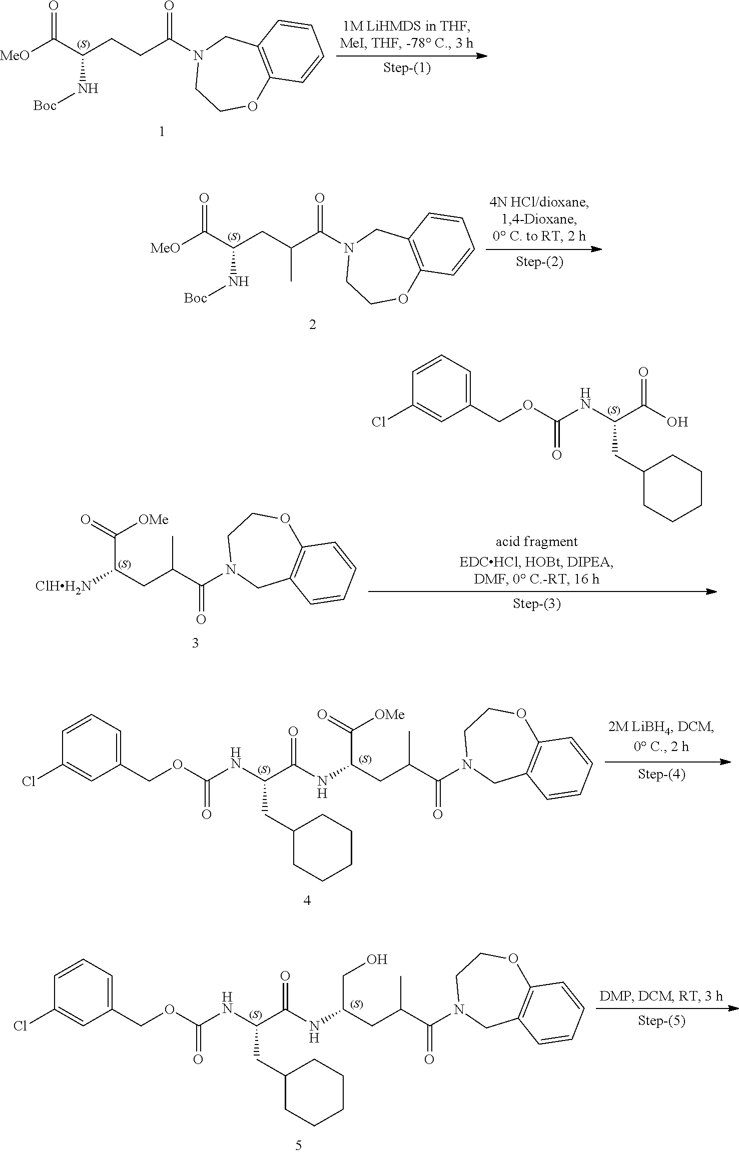

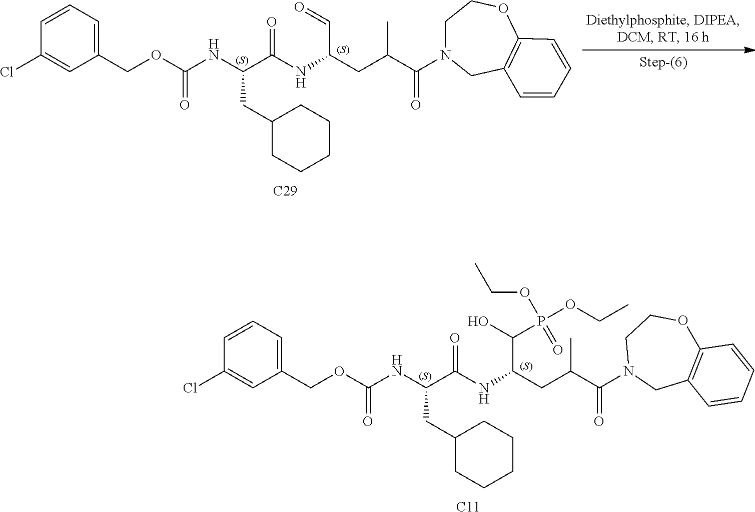

### Methyl (2S)-2-((tert-butoxycarbonyl) amino)-5-(2,3-dihydrobenzo[f][1, 4] oxazepin-4(5H)-yl)-4-methyl-5-oxopentanoate (2)

To a stirred solution of methyl (S)-2-((tert-butoxycarbonyl)amino)-5-(2,3-dihydrobenzo[f] [1,4]oxazepin-4(5H)-yl)-5-oxopentanoate (1) (2.0 g, 5.08 mmol) in THF (30 mL) added 1M LHMDS (10.7 mL, 10.01 mmol) at −78° C. and stirred for 1 h then added methyl iodide (1.2 mL, 20.32 mmol) in THF and stirred at −78° C. for 2 h. Reaction mixture was quenched with sat. Ammonium chloride solution and extracted with ethyl acetate (2×40 mL), dried over sodium sulfate and evaporated under reduced pressure. The crude residue was purified by NP, compound eluted at 30% ethyl acetate in pet ether to afford methyl (2S)-2-((tert-butoxycarbonyl) amino)-5-(2,3-dihydrobenzo[f][1,4]oxazepin-4(5H)-yl)-4-methyl-5-oxopentanoate (2). TLC system: 50% Ethyl acetate in hexane Rf: 0.4 LCMS (ESI): m/z 407.41 [M+H]⁺

### Methyl (2S)-2-amino-5-(2,3-dihydrobenzo[f][1, 4] oxazepin-4(5H)-yl)-4-methyl-5-oxopentanoate hydrochloride (3)

To a stirred solution of methyl (2S)-2-((tert-butoxycarbonyl)amino)-5-(2,3-dihydrobenzo[f] [1,4]oxazepin-4(5H)-yl)-4-methyl-5-oxopentanoate (4) (2.2 g, 7.18 mmol) in 1,4-dioxane (20 mL) was added 4N HCl in dioxane (20 mL) with drop wise at 0° C. Reaction mixture was stirred at RT for 2 h. The progress of the reaction was monitored by TLC. After consumption of starting material, the reaction mixture was evaporated under reduced pressure to obtained crude compound, the resulting crude triturated with diethyl ether to afford methyl (2S)-2-amino-5-(2,3-dihydrobenzo[f][1,4]oxazepin-4(5H)-yl)-4-methyl-5-oxopentanoate hydrochloride (3). TLC system: 10% methanol in DCM Rf: 0.1 LCMS (ESI): m/z 307.36 [M+H]⁺

### Methyl (2S)-2-((S)-2-((((3-chlorobenzyl) oxy) carbonyl) amino)-3-cyclohexylpropanamido)-5-(2,3-dihydrobenzo[f][1,4]oxazepin-4(5H)-yl)-4-methyl-5-oxopentanoate (4)

To a stirred solution of (S)-2-((((3-chlorobenzyl)oxy)carbonyl)amino)-3-cyclohexylpropanoic acid (acid fragment) (1.0 g, 2.98 mmol) DMF (15 mL) added EDC.HCl (0.84 g, 4.47 mmol), HOBt (0.59 g, 4.47 mmol), DIPEA (1.6 mL, 8.94 mmol) and methyl (2S)-2-amino-5-(2,3-dihydrobenzo[f][1,4]oxazepin-4(5H)-yl)-4-methyl-5-oxopentanoate hydrochloride (3) (1 g, 3.27 mmol) at 0° C. simultaneously and stirred at room temperature for 16 h. The progress of the reaction was monitored by TLC and LCMS. The reaction mixture was diluted with ice water (40 mL), extracted with ethyl acetate (2×40 mL), dried over sodium sulfate and evaporated under reduced pressure. The crude residue was purified by grace NP, compound eluted at 2% methanol in dichloromethane to afford methyl (2S)-2-((S)-2-((((3-chlorobenzyl)oxy)carbonyl)amino)-3-cyclohexylpropanamido)-5-(2,3-dihydrobenzo[f][1,4]oxazepin-4(5H)-yl)-4-methyl-5-oxopentanoate (4). TLC system: 5% Methanol in dichloromethane Rf: 0.3 LCMS (ESI): m/z 628.59 [M+H]⁺

### 3-Chlorobenzyl ((2S)-3-cyclohexyl-1-(((2S)-5-(2,3-dihydrobenzo[f][1,4]oxazepin-4(5H)-yl)-1-hydroxy-4-methyl-5-oxopentan-2-yl)amino)-1-oxopropan-2-yl)carbamate (5)

To a stirred solution of methyl (2S)-2-((S)-2-((((3-chlorobenzyl)oxy)carbonyl)amino)-3-cyclohexylpropanamido)-5-(2,3-dihydrobenzo[f][1,4]oxazepin-4(5H)-yl)-4-methyl-5-oxopentanoate (4) (1.2 g, 1.91 mmol) in DCM (15 mL) was added 2M LiBH₄ in THF (1.4 mL, 2.86 mmol) at 0° C. and the reaction mixture stirred for 2 h at 0° C. The progress of the reaction was monitored by TLC and LCMS. Reaction mixture was quenched with sat. ammonium chloride solution (30 mL) and extracted with DCM (2×30 mL). Organic layer was washed with brine solution (30 mL), dried over Na₂SO₄ and concentrated to afford 3-chlorobenzyl ((2S)-3-cyclohexyl-1-(((2S)-5-(2,3-dihydrobenzo[f][1,4]oxazepin-4(5H)-yl)-1-hydroxy-4-methyl-5-oxopentan-2-yl)amino)-1-oxopropan-2-yl)carbamate (5). TLC system: 5% Methanol in dichloromethane Rf: 0.2 LCMS (ESI): m/z 600.56 [M+H]⁺

### 3-Chlorobenzyl ((2S)-3-cyclohexyl-1-(((2S)-5-(2,3-dihydrobenzo[f][1,4]oxazepin-4(5H)-yl)-4-methyl-1,5-dioxopentan-2-yl)amino)-1-oxopropan-2-yl)carbamate (C29)

To a stirred solution of 3-chlorobenzyl ((2S)-3-cyclohexyl-1-(((2S)-5-(2,3-dihydrobenzo[f] [1,4]oxazepin-4(5H)-yl)-1-hydroxy-4-methyl-5-oxopentan-2-yl)amino)-1-oxopropan-2-yl)carbamate (5) (150 mg, 0.25 mmol) was dissolved in dichloromethane (5 mL) was added Dess-Martin periodinane (318 mg, 0.751 mmol) at 0° C. and stirred at RT for 3 h. Reaction mixture was diluted with DCM (10 mL) followed by sat. Hypo solution (3×15 mL), followed by sat. NaHCO₃ solution (3×15 mL). Organic layer was dried over anhydrous Na₂SO₄, filtered and concentrated to get crude compound. The crude compound was purified by prep HPLC to afford 3-chlorobenzyl ((2S)-3-cyclohexyl-1-(((2S)-5-(2,3-dihydrobenzo[f][1,4]oxazepin-4(5H)-yl)-4-methyl-1,5-dioxopentan-2-yl)amino)-1-oxopropan-2-yl) carbamate (C29). TLC system: 5% Methanol in dichloromethane Rf: 0.5 LCMS (ESI): m/z 598.28 (M+H)⁺

### 3-Chlorobenzyl ((2S)-3-cyclohexyl-1-(((2S)-1-(diethoxyphosphoryl)-5-(2,3-dihydrobenzo[f] [1, 4] oxazepin-4 (5H)-yl)-1-hydroxy-4-methyl-5-oxopentan-2-yl) amino)-1-oxopropan-2-yl) carbamate (C11)

To a stirred solution of 3-chlorobenzyl ((2S)-3-cyclohexyl-1-(((2S)-5-(2,3-dihydrobenzo[f][1,4]oxazepin-4(5H)-yl)-4-methyl-1,5-dioxopentan-2-yl)amino)-1-oxopropan-2-yl)carbamate (C29) (250 mg crude, 0.41 mmol) in DCM (10 mL) added DIPEA (0.22 mL, 1.23 mmol) followed by added diethyl phosphite (0.17 mL, 1.23 mmol) and the reaction mixture stirred at RT for 16 h. The progress of the reaction was monitored by TLC and LCMS. After 16 h, the reaction mixture quenched with ammonium chloride (15 mL) and extracted with DCM (2×20 mL). Combined organic layer was dried over anhydrous Na₂SO₄, and evaporated to afford crude residue. It was purified prep HPLC to afford 3-chlorobenzyl ((2S)-3-cyclohexyl-1-(((2S)-1-(diethoxyphosphoryl)-5-(2,3-dihydrobenzo[f][1,4]oxazepin-4(5H)-yl)-1-hydroxy-4-methyl-5-oxopentan-2-yl)amino)-1-oxopropan-2-yl)carbamate (C11). TLC system: 5% Methanol in dichloromethane Rf: 0.4 LCMS (ESI): m/z 736.54 (M+H)⁺

### Example 7: Synthesis of Compound C23 and C13

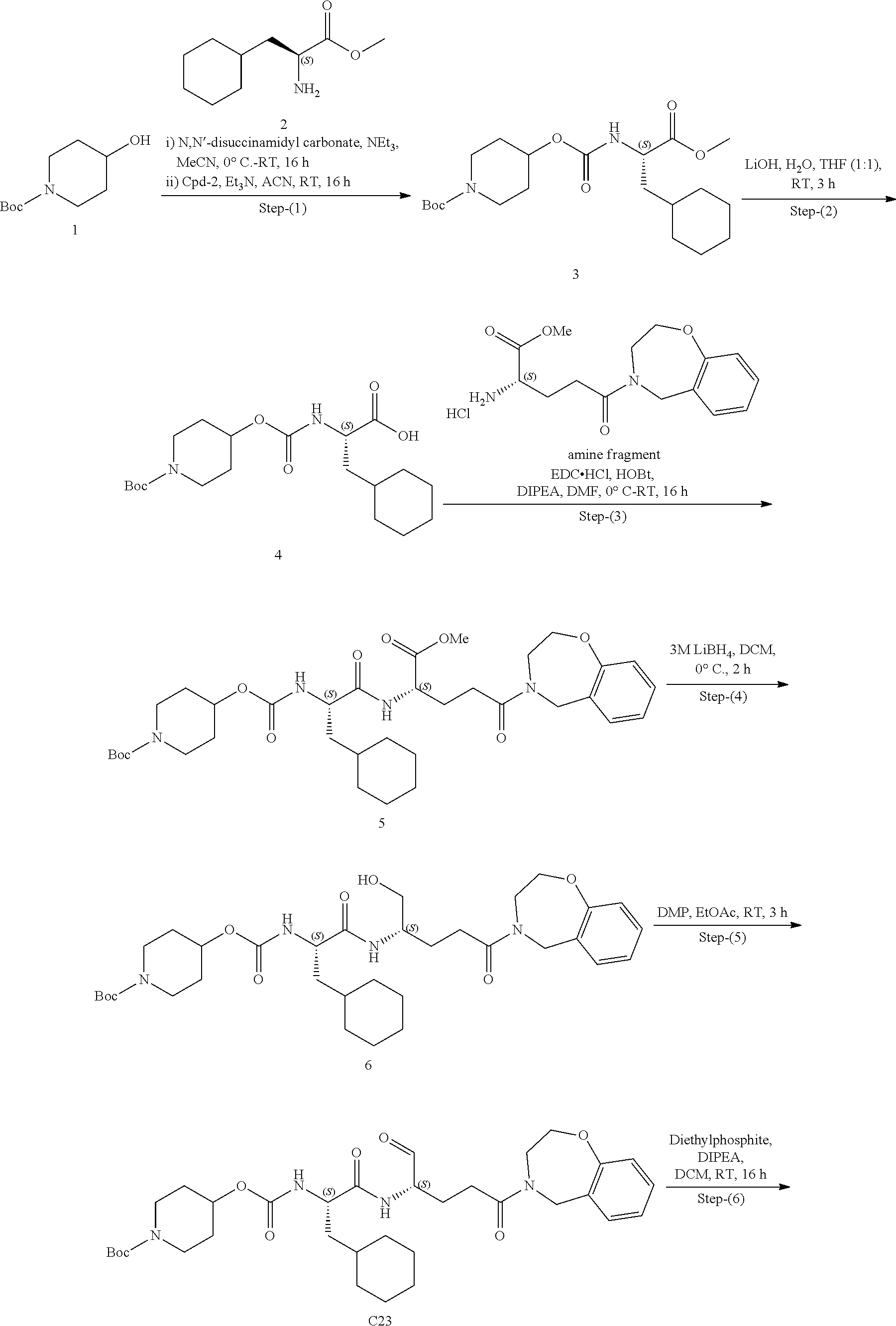

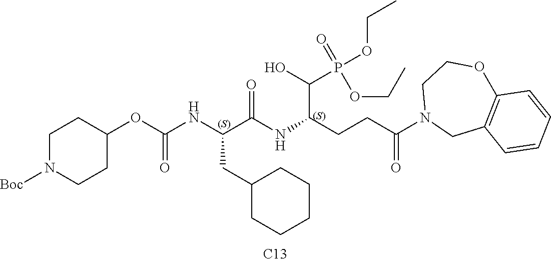

### Tert-butyl (S)-4-(((3-cyclohexyl-1-methoxy-1-oxopropan-2-yl) carbamoyl) oxy) piperidine-1-carboxylate (3)

To a stirred solution of tert-butyl 4-hydroxypiperidine-1-carboxylate (1) (300 mg, 1.49 mmol) in ACN (5 mL) was added N,N′ disuccinimidyl carbonate (572 mg, 2.23 mmol), followed by triethylamine (0.62 mL, 4.47 mmol) at room temperature and stirred for 16 h. The progress of the reaction was monitored by TLC. The reaction mass was used directly in the subsequent reaction.

In another RB flask, methyl (S)-2-amino-3-cyclohexylpropanoate (2) (250 mg, 1.12 mmol) was taken in ACN (5 mL), and treated with triethylamine (0.3 mL, 2.25 mmol). The resulting reaction mixture was stirred for 5 min, then added above prepared reaction mass drop-wise and the reaction mixture stirred at room temperature for 16 h. After 16 h, the reaction mixture was quenched with ice water (15 mL) and extracted with ethyl acetate (2×15 mL), combined organic layers were washed with brine solution (20 mL), dried over sodium sulfate and evaporated under reduced pressure. The crude residue was purified by normal phase chromatography to afford tert-butyl (S)-4-(((3-cyclohexyl-1-methoxy-1-oxopropan-2-yl) carbamoyl) oxy) piperidine-1-carboxylate (3). TLC system: 50% Ethyl acetate in Pet ether Rf: 0.3 LCMS (ESI): m/z 435.2[M+Na]⁻

### (S)-2-((((1-(tert-butoxycarbonyl) piperidin-4-yl) oxy) carbonyl) amino)-3-cyclohexylpropanoic acid (4)

To a stirred solution of tert-butyl (S)-4-(((3-cyclohexyl-1-methoxy-1-oxopropan-2-yl) carbamoyl) oxy) piperidine-1-carboxylate (3) (0.35 g, 0.84 mmol) in THF (10 mL), water (10 mL) was added lithium hydroxide (106 mg, 2.54 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (S)-2-((((1-(tert-butoxycarbonyl) piperidin-4-yl) oxy) carbonyl) amino)-3-cyclohexylpropanoic acid (4). TLC system: 20% Ethyl acetate in Pet ether Rf: 0.1 LCMS (ESI): m/z=421.39 [M+Na]⁺

### Tert-butyl 4-((((S)-3-cyclohexyl-1-(((S)-5-(2,3-dihydrobenzo[f][1, 4] oxazepin-4(5H)-yl)-1-methoxy-1,5-dioxopentan-2-yl) amino)-1-oxopropan-2-yl)carbamoyl)oxy)piperidine-1-carboxylate (5)

To a stirred solution of (S)-2-((((1-(tert-butoxycarbonyl)piperidin-4-yl)oxy)carbonyl)amino)-3-cyclohexylpropanoic acid (4) (1.5 g, 3.64 mmol) in DMF (20 mL) added EDC.HCl (1.04 g, 5.46 mmol), HOBT (0.73 g, 5.46 mmol), DIPEA (1.9 mL, 10.92 mmol) and methyl (S)-2-amino-5-(2,3-dihydrobenzo[f][1,4]oxazepin-4(5H)-yl)-5-oxopentanoatehydrochloride (amine fragment) (1.27 g, 4.36 mmol) at 0° C. simultaneously and stirred at room temperature for 16 h. The progress of the reaction was monitored by TLC and LCMS. After 16 h, reaction mixture was quenched with ice water (50 mL), obtained solids were filtered and washed with excess water then dried under vacuum to afford tert-butyl 4-((((S)-3-cyclohexyl-1-(((S)-5-(2,3-dihydrobenzo[f][1,4]oxazepin-4(5H)-yl)-1-methoxy-1,5-dioxopentan-2-yl)amino)-1-oxopropan-2-yl)carbamoyl)oxy)piperidine-1-carboxylate (5). TLC system: 5% Methanol in DCM R_{f}: 0.3 LCMS (ESI): m/z 673.46 [M+H]⁺

### Tert-butyl 4-((((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamoyl)oxy)piperidine-1-carboxylate (6)

To a stirred solution of tert-butyl 4-((((S)-3-cyclohexyl-1-(((S)-5-(2,3-dihydrobenzo[f][1,4]oxazepin-4(5H)-yl)-1-methoxy-1,5-dioxopentan-2-yl)amino)-1-oxopropan-2-yl) carbamoyl) oxy) piperidine-1-carboxylate (5) (1.5 g, 2.23 mmol) in DCM (15 mL) was added 2M LiBH₄ in THF (2.2 mL, 4.46 mmol) at 0° C. and the reaction mixture stirred for 2 h at 0° C. The progress of the reaction was monitored by TLC and LCMS. Then reaction mixture was quenched with sat. NH₄Cl solution (30 mL) and extracted with ethyl acetate (2×30 mL). Organic layer was washed with brine solution (30 mL), dried over Na₂SO₄ and concentrated to get crude compound. It was triturated with diethyl ether to afford tert-butyl 4-((((S)-3-cyclohexyl-1-(((S)-5-(2,3-dihydrobenzo[f][1,4]oxazepin-4(5H)-yl)-1-hydroxy-5-oxopentan-2-yl) amino)-1-oxopropan-2-yl) carbamoyl) oxy)piperidine-1-carboxylate (6). TLC system: 5% Methanol in DCM R_{f}: 0.2 LCMS (ESI): m/z 645.67 [M+H]⁺

### Tert-butyl 4-((((S)-3-cyclohexyl-1-(((S)-5-(2,3-dihydrobenzo[f][1, 4] oxazepin-4(5H)-yl)-1,5-dioxopentan-2-yl) amino)-1-oxopropan-2-yl)carbamoyl)oxy)piperidine-1-carboxylate (C23)

To a stirred solution of tert-butyl 4-((((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamoyl)oxy)piperidine-1-carboxylate (6) (200 mg, 0.31 mmol) was dissolved in ethyl acetate (5 mL) was added Dess-Martin periodinane (395 mg, 0.93 mmol) at 0° C. and stirred at RT for 3 h. Reaction mixture was diluted with ethyl acetate (10 mL) and washed with sat. Hypo solution (3×20 mL), sat. NaHCO₃ solution (3×20 mL). Organic layer was dried over anhydrous Na₂SO₄, filtered and concentrated to afford crude product, this residue was purified by normal phase chromatography by eluting 3% methanol in dichloromethane to afford tert-butyl 4-((((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamoyl)oxy)piperidine-1-carboxylate (C23). TLC system: 5% Methanol in DCM Rf: 0.4 LCMS (ESI): m/z 643.68 (M+H)⁺

### Tert-butyl 4-((((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamoyl)oxy)piperidine-1-carboxylate (C13)

To a stirred solution of tert-butyl 4-((((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamoyl)oxy)piperidine-1-carboxylate (C23) (200 mg crude, 0.31 mmol) in DCM (10 mL) added DIPEA (0.16 mL, 0.93 mmol) followed by added diethyl phosphite (0.13 mL, 0.93 mmol) and the reaction mixture stirred at RT for 16 h. The progress of the reaction was monitored by TLC and LCMS. After 16 h, the reaction mixture quenched with Sat. ammonium chloride (20 mL) and extracted with DCM (2×20 mL). Combined organic layer was dried over anhydrous Na₂SO₄, and evaporated to afford crude residue. It was purified by prep HPLC to afford tert-butyl 4-((((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamoyl)oxy)piperidine-1-carboxylate (C13). TLC system: 5% Methanol in DCM R_{f}: 0.4 LCMS (ESI): m/z 781.71 [M+H]⁺

### Example 8: Synthesis of Compounds C24 and C14

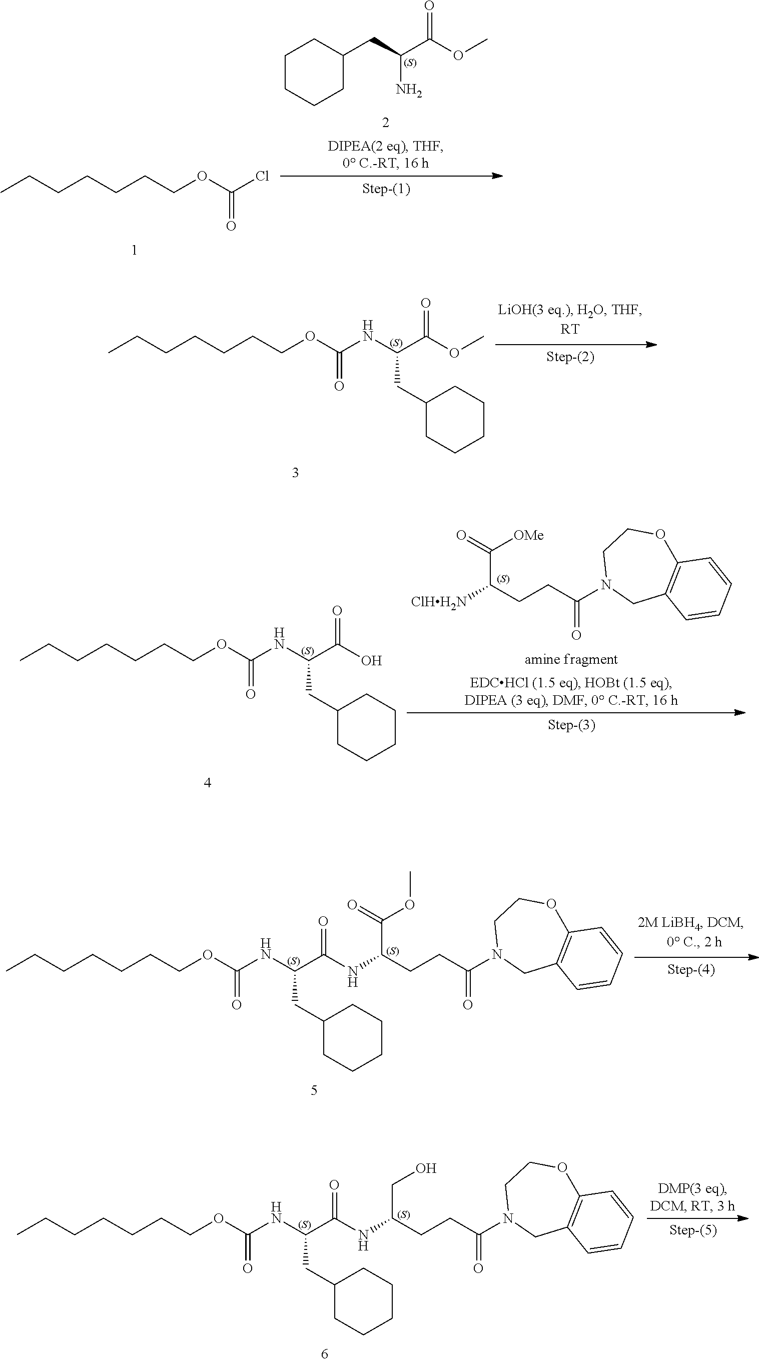

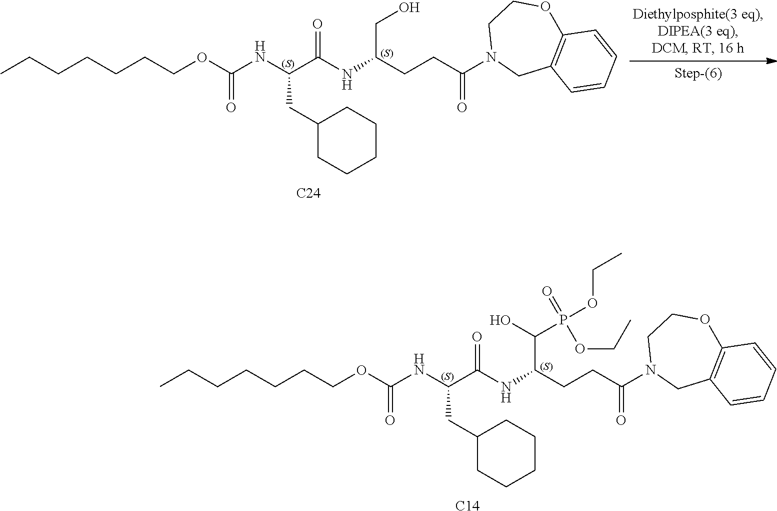

### Methyl (S)-3-cyclohexyl-2-(((heptyloxy)carbonyl)amino)propanoate (3)

To a stirred solution of (S)-2-amino-3-cyclohexylpropanoate hydrochloride (2) (2.97 g, 13.48 mmol), in THF (20 mL) and DIPEA (5.8 mL, 33.70 mmol) at 0° C. was added heptyl carbonochloridate (1) (2 g, 11.235 mmol). The resulting mixture was stirred at RT for 2 h. The progress of the reaction was monitored by TLC and LCMS. After 2 h, reaction mixture was quenched with water (50 mL) and extracted with ethyl acetate (2×80 mL), the combined organic layer was dried over sodium sulfate, filtered and evaporated under reduced pressure. The crude residue was purified by silica gel column by eluting with 50% ethyl acetate in pet ether to afford methyl (S)-3-cyclohexyl-2-(((heptyloxy)carbonyl)amino)propanoate (3). TLC system: 20% EtOAc in Pet Ether R_{f}: 0.55 LCMS (ESI): m/z 328.49 (M+H)⁺

### (S)-3-cyclohexyl-2-(((heptyloxy)carbonyl)amino)propanoic acid (4)

To a stirred solution of methyl (S)-3-cyclohexyl-2-(((heptyloxy)carbonyl)amino)propanoate (3) (1.2 g, 3.66 mmol) in THF (12 mL) and water (6 mL), was added lithium hydroxide (264 mg, 11.009 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH˜4, and extracted with dichloromethane (2×30 mL), dried over sodium sulfate, concentrated under reduced pressure to afford (S)-3-cyclohexyl-2-(((heptyloxy)carbonyl)amino)propanoic acid (4). TLC system: 5% Methanol in DCM R_{f}: 0.2 LCMS (ESI): m/z 314.2 (M+H)⁺

### Methyl (S)-2-((S)-3-cyclohexyl-2-(((heptyloxy)carbonyl)amino)propanamido)-5-(2,3-dihydrobenzo[f][1,4]oxazepin-4(5H)-yl)-5-oxopentanoate (5)

At 0° C., to a stirred solution of (S)-3-cyclohexyl-2-(((heptyloxy)carbonyl)amino)propanoic acid (1 g, 3.18 mmol) in DMF (20 mL) was added EDC.HCl (0.91 g, 4.7 mmol), HOBT (660 mg, 4.7 mmol), DIPEA (1.2 mL, 9.5 mmol) and methyl (S)-2-amino-5-(2,3-dihydrobenzo[f][1,4]oxazepin-4(5H)-yl)-5-oxopentanoate hydrochloride (Amine fragment) (1.26 g, 3.8 mmol) and stirred at room temperature for 16 h. The progress of the reaction was monitored by TLC and LCMS. After 16 h, reaction mixture was quenched with ice water (500 mL), extracted with ethyl acetate (2×50 mL), the combined organic layer was dried over sodium sulfate and evaporated under reduced pressure. The crude residue was purified by silica gel column by eluting with 40% ethyl acetate in pet ether to afford methyl (S)-2-((S)-3-cyclohexyl-2-(((heptyloxy)carbonyl)amino)propanamido)-5-(2,3-dihydrobenzo[f][1,4]oxazepin-4(5H)-yl)-5-oxopentanoate (5). TLC system: 5% Methanol in DCM R_{f}: 0.4 LCMS (ESI): m/z 588.68 (M+H)⁺

### Heptyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6)

To a stirred solution of methyl (S)-2-((S)-3-cyclohexyl-2-(((heptyloxy)carbonyl)amino)propanamido)-5-(2,3-dihydrobenzo[f][1,4]oxazepin-4(5H)-yl)-5-oxopentanoate (5) (600 mg, 1.01 mmol) in DCM (12 mL) was added 2M LiBH₄ in THF (0.76 mL, 1.52 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford the crude hexyl heptyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6). TLC system: 5% MeOH in DCM R_{f} 0.3 LCMS (ESI): m/z 560.3 (M+H)⁺

### Heptyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C24)

To a stirred solution of heptyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6) (180 mg, 0.32 mmol) in DCM (5 mL) was added Dess-Martin periodinane (410 mg, 0.96 mmol) at 0° C. and stirred at RT for 3 h. The progress of the reaction was monitored by TLC and LCMS. Reaction mixture was diluted with DCM (50 mL) and washed with sat. NaHCO₃ solution (3×20 mL) followed by sat. Hypo solution (3×20 mL). Organic layer was dried over anhydrous Na₂SO₄, filtered and concentrated to get crude heptyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C24). TLC system: 5% Methanol in DCM R_{f}: 0.5 LCMS (ESI): m/z 558.52 (M+H)⁺

### Heptyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (Compound C14)

To a stirred solution of hexyl heptyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (249 mg, 0.446 mmol) in DCM (5 mL) was added DIPEA (0.177 mL, 1.33 mmol) followed by diethylphosphate (0.18 mL, 1.33 mmol) and the reaction mixture stirred at RT for 16 h. The progress of the reaction was monitored by TLC and LCMS. After 16 h, the reaction mixture quenched with ammonium chloride (15 mL) and extracted with DCM (2×15 mL). Combined organic layer was dried over anhydrous Na₂SO₄, and evaporated to afford crude residue. It was purified prep HPLC to afford heptyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (Compound C14). TLC system: 5% MeOH in DCM R_{f}: 0.5 LCMS (ESI): m/z 696.70 (M+H)⁺

### Example 9: Synthesis of Compound C17 and C5

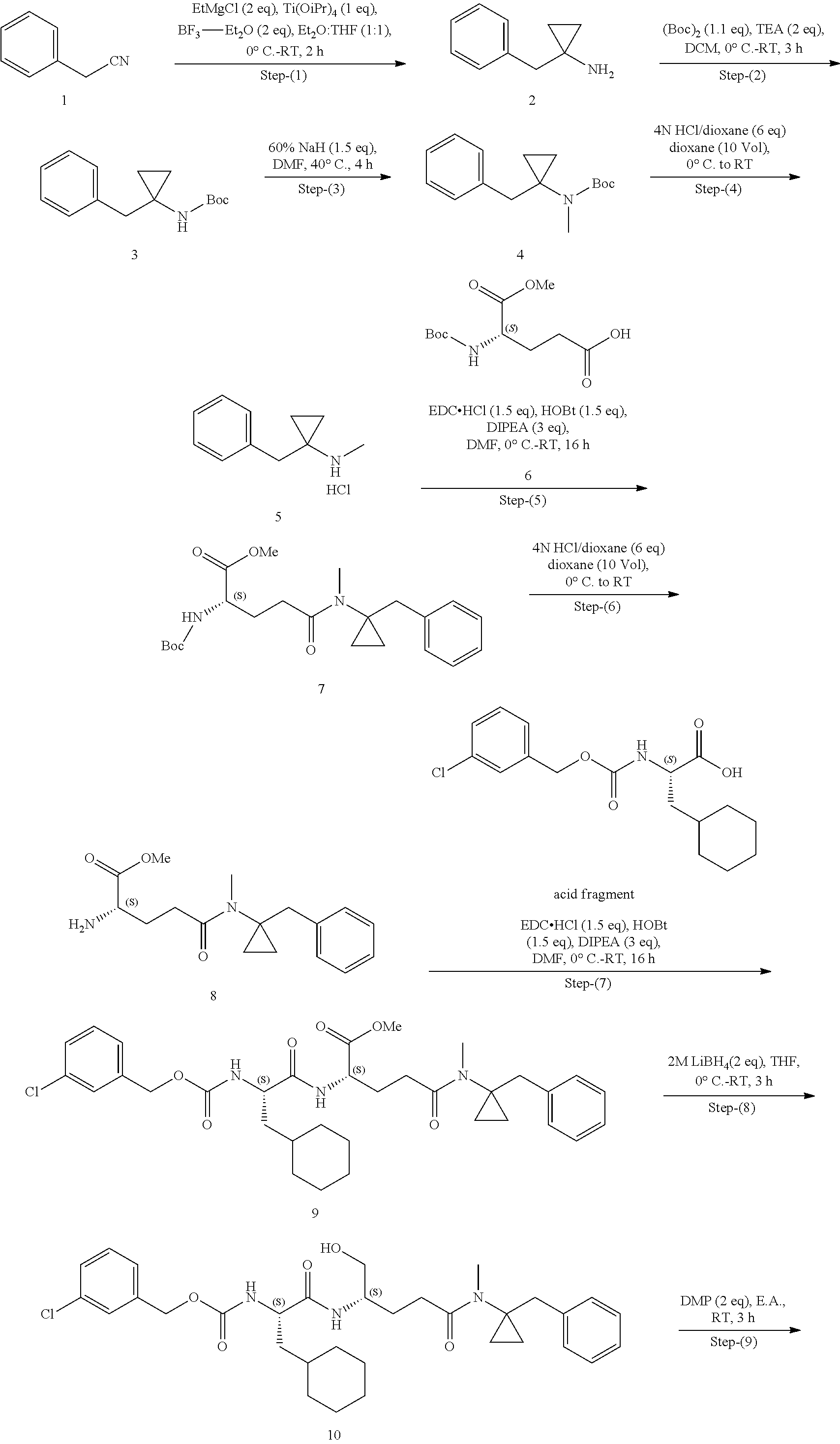

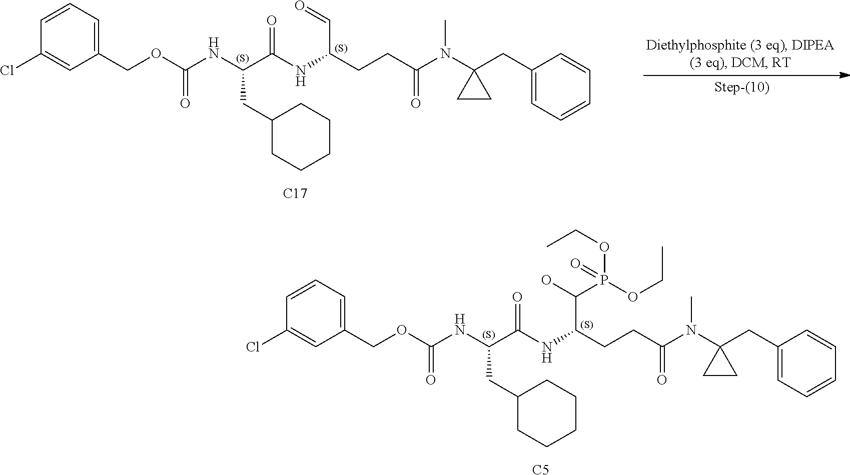

### 1-benzylcyclopropan-1-amine (2)

To a stirred solution of 2-phenylacetonitrile (1) (2 g, 17.094 mmol) in Et₂O:THF (1:1) (20 mL) were added Titanium iso propoxide (5.14 g, 18.119 mmol) and followed by added 2M Ethyl magnesium chloride in THF (17 mL, 34.188 mmol) slowly drop wise for 10 min at 0° C. Then the reaction mixture stirred at RT for 1 h and then added BF₃-Et₂O (4.8 mL, 34.188 mmol) slowly at 0° C. for 15 min (exothermic occurred) and stirred at room temperature for 1 h. The progress of the reaction was monitored by TLC and LCMS. After 1 h, the reaction mixture was poured in 10% NaOH solution (100 mL) white precipitate formed. The reaction mixture filtered through celite bed and washed with ethyl acetate (50 mL) and filtrate washed with brine solution (100 ml) and combined organic layer was dried over sodium sulfate, filtered and evaporated under reduced pressure. The crude residue was purified by combi-flash compound eluted at 70% Ethyl acetate in pet ether to afford 1-benzylcyclopropan-1-amine (2). TLC system: 80% Ethyl acetate in Pet ether Rf: 0.3 LCMS (ESI): m/z 148.11[M+H]+

### Tert-butyl (1-benzylcyclopropyl) carbamate (3)

To a stirred solution of 1-benzylcyclopropan-1-amine (2) (4.2 g, 28.5714 mmol) in DCM (50 mL) were added TEA (4.1 mL, 57.142 mmol) and followed by added Boc anhydride (6.8 mL, 31.428 mmol) slowly drop wise for 10 min at 0° C. Then the reaction mixture stirred at RT for 3 h. The progress of the reaction was monitored by TLC and LCMS. After 3 h, the reaction mixture was diluted with ice water (50 mL), extracted with DCM (2×50 mL). The combined organic layer was dried over sodium sulfate, filtered and evaporated under reduced pressure. The crude residue was purified by combi-flash compound eluted at 5% Ethyl acetate in pet ether to afford tert-butyl (1-benzylcyclopropyl) carbamate (3). TLC system: 30% Ethyl acetate in Pet ether Rf: 0.8 LCMS (ESI): m/z 148.11 [M−Boc]⁺

### Tert-butyl (1-benzylcyclopropyl)(methyl)carbamate (4)

To a stirred solution of tert-butyl (1-benzylcyclopropyl) carbamate (3) (2×2.5 g, 10.121 mmol) in DMF (25 mL) in sealed vessel added 60% NaH (607 mg, 15.182 mmol) at 0° C. and stirred for 15 min after added methyl iodide (2.6 mL, 40.485 mmol) slowly drop wise for 10 min at 0° C. Then the reaction mixture heated to 40° C. for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure to afford tert-butyl (1-benzylcyclopropyl)(methyl)carbamate (4). TLC system: 5% Ethyl acetate in Pet ether Rf: 0.6 LCMS (ESI): m/z 162.11 [M−Boc]⁺

### 1-benzyl-N-methylcyclopropan-1-amine hydrochloride (5)

To a stirred solution of tert-butyl (1-benzylcyclopropyl)(methyl)carbamate (4) (1.3 g, 4.9808 mmol in 1,4-dioxane (10 mL) was added 4N HCl in dioxane (20 mL) with drop wise at 0° C. and the reaction mixture was stirred at RT for 3 h. The progress of the reaction was monitored by TLC and LCMS. After consumption of starting material, the reaction mixture was evaporated under reduced pressure to obtained crude compound, the resulting crude triturated with diethyl ether to afford 1-benzyl-N-methylcyclopropan-1-aminehydrochloride (5). TLC system: 5% Methanol in DCM Rf: 0.2 LCMS (ESI): m/z 162.32 [M+H]⁺

### Methyl N5-(1-benzylcyclopropyl)-N2-(tert-butoxycarbonyl)-N5-methyl-L-glutaminate (7)

To a stirred solution of (S)-4-((tert-butoxycarbonyl)amino)-5-methoxy-5-oxopentanoic acid (6) (1 g, 3.831 mmol) in DMF (10 mL) were added EDC.HCl (1.1 g, 5.747 mmol), HOBT (775 mg, 5.747 mmol), DIPEA (2.11 mL, 11.494 mmol) and 1-benzyl-N-methylcyclopropan-1-aminehydrochloride (5) (678 mg, 4.214 mmol) at 0° C. simultaneously and stirred at room temperature for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 40% Ethyl acetate in pet ether to afford methyl N5-(1-benzylcyclopropyl)-N2-(tert-butoxycarbonyl)-N5-methyl-L-glutaminate (7). TLC system: 50% Ethyl acetate in Pet ether Rf: 0.5 LCMS (ESI): m/z 305.19 [M−Boc]⁺

### Methyl N5-(1-benzylcyclopropyl)-N5-methyl-L-glutaminate hydrochloride (8)

To a stirred solution of methyl N5-(1-benzylcyclopropyl)-N2-(tert-butoxycarbonyl)-N5-methyl-L-glutaminate (7) (1.0 g, 2.475 mmol) in 1,4-dioxane (10 mL) was added 4N HCl in dioxane (20 mL) with drop wise at 0° C. and the reaction mixture was stirred at RT for 3 h. The progress of the reaction was monitored by TLC and LCMS. After consumption of starting material, the reaction mixture was evaporated under reduced pressure to obtained crude compound, the resulting crude triturated with diethyl ether to afford methyl N5-(1-benzylcyclopropyl)-N5-methyl-L-glutaminate hydrochloride (8). TLC system: 5% Methanol in DCM Rf: 0.2 LCMS (ESI): m/z 305.26 [M+H]⁺

### Methyl N5-(1-benzylcyclopropyl)-N2-((S)-2-((((3-chlorobenzyl)oxy)carbonyl)amino)-3-cyclohexylpropanoyl)-N5-methyl-L-glutaminate (9)

To a stirred solution of (S)-2-((((3-chlorobenzyl)oxy)carbonyl)amino)-3-cyclohexylpropanoic acid (acid fragment) (1.0 g, 2.949 mmol) DMF (10 mL) added EDC.HCl (845 mg, 4.424 mmol), HOBT (597 mg, 5.899 mmol), DIPEA (1.63 mL, 8.849 mmol) and methyl N5-(1-benzylcyclopropyl)-N5-methyl-L-glutaminate hydrochloride (8) (986 mg, 3.244 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×50 mL). The combined organic layer was dried over sodium sulfate, filtered and evaporated under reduced pressure. The crude residue was purified by combi-flash, compound eluted at 60% Ethyl acetate in pet ether to afford methyl N5-(1-benzylcyclopropyl)-N2-((S)-2-((((3-chlorobenzyl)oxy)carbonyl)amino)-3-cyclohexylpropanoyl)-N5-methyl-L-glutaminate (9). TLC system: 50% Ethyl acetate in Pet ether Rf: 0.5 LCMS (ESI): m/z 626.58 [M+H]⁺

### 3-Chlorobenzyl ((S)-1-(((S)-5-((1-benzylcyclopropyl)(methyl)amino)-1-hydroxy-5-oxopentan-2-yl)amino)-3-cyclohexyl-1-oxopropan-2-yl)carbamate (10)

To a stirred solution of methyl N5-(1-benzylcyclopropyl)-N2-((S)-2-((((3-chlorobenzyl)oxy)carbonyl)amino)-3-cyclohexylpropanoyl)-N5-methyl-L-glutaminate (9) (1 g, 1.6 mmol) in THF (10 mL) was added 2M LiBH₄ in THF (1.6 mL, 3.2 mmol) at 0° C. and the reaction mixture stirred for 2 h at 0° C. The progress of the reaction was monitored by TLC and LCMS. Then reaction mixture was quenched with water (10 mL) and extracted with ethyl acetate (2×30 mL). Organic layer was washed with brine solution (10 mL), dried over Na₂SO₄ and concentrated to get crude compound. It was purified combi-flash, compound eluted at 80% Ethyl acetate in pet ether to afford 3-chlorobenzyl ((S)-1-(((S)-5-((1-benzylcyclopropyl)(methyl)amino)-1-hydroxy-5-oxopentan-2-yl)amino)-3-cyclohexyl-1-oxopropan-2-yl)carbamate (10). TLC system: 100% Ethyl acetate Rf: 0.2 LCMS (ESI): m/z 598.98 (M+H)⁺

### 3-Chlorobenzyl ((S)-1-(((S)-5-((1-benzylcyclopropyl)(methyl)amino)-1,5-dioxopentan-2-yl)amino)-3-cyclohexyl-1-oxopropan-2-yl)carbamate (Compound C17)

To a stirred solution of 3-chlorobenzyl ((S)-1-(((S)-5-((1-benzylcyclopropyl)(methyl)amino)-1-hydroxy-5-oxopentan-2-yl)amino)-3-cyclohexyl-1-oxopropan-2-yl)carbamate (10) (150 mg, 0.2508 mmol) was dissolved in ethyl acetate (10 mL) was added Dess-Martin periodinane (320 mg, 0.752 mmol) at 0° C. and stirred at RT for 3 h. Reaction mixture was diluted with ethyl acetate (10 mL) and washed with sat. Hypo solution (3×10 mL), sat. NaHCO₃ solution (3×20 mL). Organic layer was dried over anhydrous Na₂SO₄, filtered and concentrated to get crude compound. It was purified combi-flash, compound eluted at 20% MeOH in DCM to afford 3-chlorobenzyl ((S)-1-(((S)-5-((1-benzylcyclopropyl)(methyl)amino)-1,5-dioxopentan-2-yl)amino)-3-cyclohexyl-1-oxopropan-2-yl)carbamate (Compound C17). TLC system: 80% Ethyl acetate in pet ether Rf: 0.4 LCMS (ESI): m/z 596.44 (M+H)⁺

### 3-Chlorobenzyl ((2S)-1-(((2S)-5-((1-benzylcyclopropyl)(methyl)amino)-1-(diethoxyphosphoryl)-1-hydroxy-5-oxopentan-2-yl)amino)-3-cyclohexyl-1-oxopropan-2-yl)carbamate (Compound C5)

To a stirred solution of 3-chlorobenzyl ((S)-1-(((S)-5-((1-benzylcyclopropyl)(methyl)amino)-1,5-dioxopentan-2-yl)amino)-3-cyclohexyl-1-oxopropan-2-yl)carbamate (Compound C17) (200 mg, 0.033 mmol) in DCM (10 mL) added DIPEA (0.2 mL, 1.032 mmol) followed by added diethyl phosphite (0.14 mL, 1.032 mmol) and the reaction mixture stirred at RT for 16 h. The progress of the reaction was monitored by TLC and LCMS. After 16 h, the reaction mixture quenched with ammonium chloride (15 mL) and extracted with DCM (2×20 mL). Combined organic layer was dried over anhydrous Na₂SO₄, and evaporated to afford crude residue. It was purified by prep HPLC to afford 3-chlorobenzyl ((2S)-1-(((2S)-5-((1-benzylcyclopropyl)(methyl)amino)-1-(diethoxyphosphoryl)-1-hydroxy-5-oxopentan-2-yl)amino)-3-cyclohexyl-1-oxopropan-2-yl)carbamate (Compound C5). TLC system: 100% Ethyl acetate Rf: 0.3 LCMS (ESI): m/z 734.51 (M+H)⁺

### Example 10: Synthesis of Compounds C18 and C19

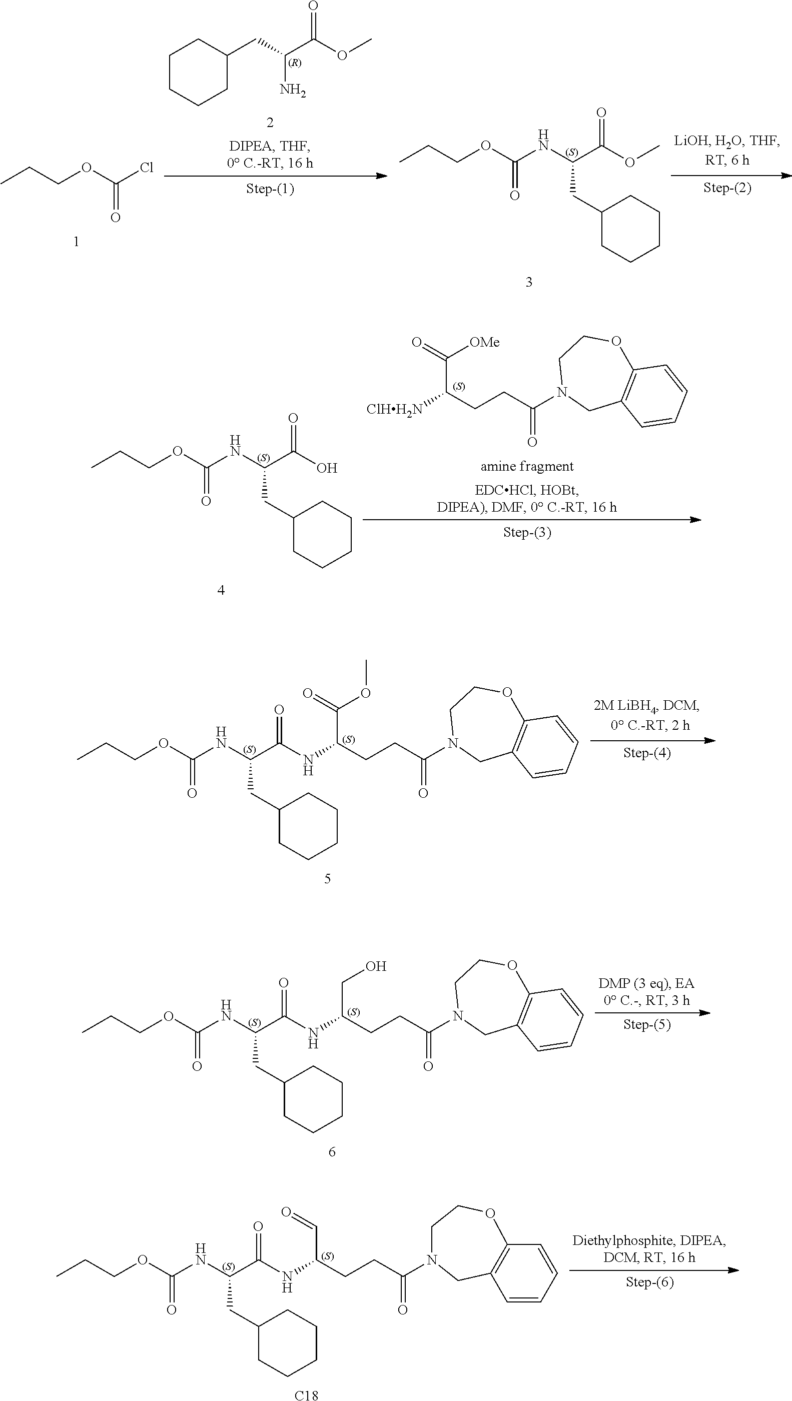

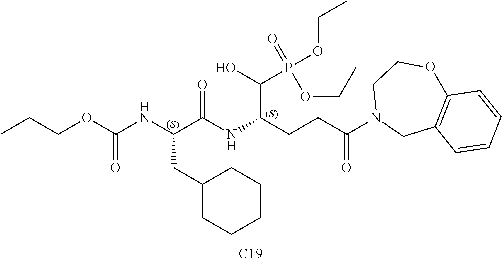

### Methyl (S)-3-cyclohexyl-2-((propoxycarbonyl)amino)propanoate (3)

To a stirred solution of (S)-2-amino-3-cyclohexylpropanoate hydrochloride (2) (4 g, 22.13 mmol) in THF (20 mL) and DIPEA (8.7 mL, 49.18 mmol) at 0° C. was added propyl carbonochloridate (1) (3 g, 24.59 mmol). The resulting mixture was stirred at RT for 2 h. The progress of the reaction was monitored by TLC and LCMS. After 2 h, reaction mixture was quenched with water (50 mL) and extracted with ethyl acetate (2×80 mL), the combined organic layer was dried over sodium sulfate, filtered and evaporated under reduced pressure. The crude residue was purified by silica gel column by eluting with 20% ethyl acetate in pet ether to afford methyl (S)-3-cyclohexyl-2-((propoxycarbonyl)amino)propanoat (3). TLC system: 50% Ethyl acetate in pet ether R_{f}: 0.55 LCMS (ESI): m/z 272.2 (M+H)⁺

### (S)-3-cyclohexyl-2-((propoxycarbonyl)amino)propanoic acid (4)

To a stirred solution of methyl (S)-3-cyclohexyl-2-((propoxycarbonyl)amino)propanoat (3) (3 g, 11.07 mmol) in THF (20 mL) and water (5 mL), was added lithium hydroxide (1.06 g, 44.28 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH˜4, and extracted with dichloromethane (2×30 mL), dried over sodium sulfate, concentrated under reduced pressure to afford (S)-3-cyclohexyl-2-((propoxycarbonyl)amino)propanoic acid (4). TLC system: 5% Methanol in DCM R_{f}: 0.2 LCMS (ESI): m/z 256.26 (M−H)⁺

### Methyl (S)-2-((S)-3-cyclohexyl-2-((propoxycarbonyl)amino)propanamido)-5-(2,3-dihydrobenzo[f][1,4]oxazepin-4(5H)-yl)-5-oxopentanoate (5)

To a stirred solution of (S)-3-cyclohexyl-2-((propoxycarbonyl)amino)propanoic acid (4) (0.6 g, 2.33 mmol) in DMF (10 mL) at 0° C. was added EDC.HCl (0.66 g, 3.501 mmol), HOBT (0.47 g, 3.5 mmol), DIPEA (1.2 mL, 6.99 mmol) and methyl (S)-2-amino-5-(2,3-dihydrobenzo[f][1,4]oxazepin-4(5H)-yl)-5-oxopentanoate hydrochloride (Amine fragment) (0.8 g, 2.33 mmol) simultaneously and stirred at room temperature for 16 h. The progress of the reaction was monitored by TLC and LCMS. After 16 h, reaction mixture was quenched with ice water (30 mL), extracted with ethyl acetate (2×60 mL), the combined organic layer was dried over sodium sulfate and evaporated under reduced pressure. The crude residue was purified by silica gel column by eluting with 40% ethyl acetate in pet ether to afford ethyl (S)-2-((S)-3-cyclohexyl-2-((propoxycarbonyl)amino)propanamido)-5-(2,3-dihydrobenzo[f][1,4]oxazepin-4(5H)-yl)-5-oxopentanoate (5). TLC system: 5% Methanol in DCM R_{f}: 0.6 LCMS (ESI): m/z 532.61 (M+H)⁺

### Propyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6)

To a stirred solution of ethyl (S)-2-((S)-3-cyclohexyl-2-((propoxycarbonyl)amino)propanamido)-5-(2,3-dihydrobenzo[f][1,4]oxazepin-4(5H)-yl)-5-oxopentanoate (5) (400 mg, 0.753 mmol) in DCM (10 mL) was added 2M LiBH₄ in THF (0.7 mL, 1.506 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford the propyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6). TLC system: 5% MeOH in DCM R_{f} 0.3 LCMS (ESI): m/z 504.64.5 (M+H)⁺

### Propyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C18)

To a stirred solution of propyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6) (230 mg, 0.45 mmol) in EA (5 mL) was added Dess-Martin periodinane (581 mg, 1.37 mmol) at 0° C. and stirred at RT for 3 h. The progress of the reaction was monitored by TLC and LCMS. Reaction mixture was diluted with DCM (50 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford propyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C18). TLC system: 5% Methanol in DCM R_{f}: 0.4 LCMS (ESI): m/z 502.46 (M+H)⁺

### Propyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (Compound C19)

To a stirred solution of propyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C18) (Crude) (200 mg, 0.199 mmol) in DCM (2 mL) was added DIPEA (0.2 mL, 0.59 mmol) followed by diethylphosphate (0.2 mL, 0.59 mmol) and the reaction mixture stirred at RT for 16 h. The progress of the reaction was monitored by TLC and LCMS. After 16 h, the reaction mixture quenched with ammonium chloride (15 mL) and extracted with DCM (2×15 mL). Combined organic layer was dried over anhydrous Na₂SO₄, and evaporated to afford crude residue. It was purified by prep HPLC to afford propyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (Compound C19). TLC system: 5% MeOH in DCM R_{f}: 0.45 LCMS (ESI): m/z 640.59 (M+H)⁺

### Example 11: Synthesis of Compound C21

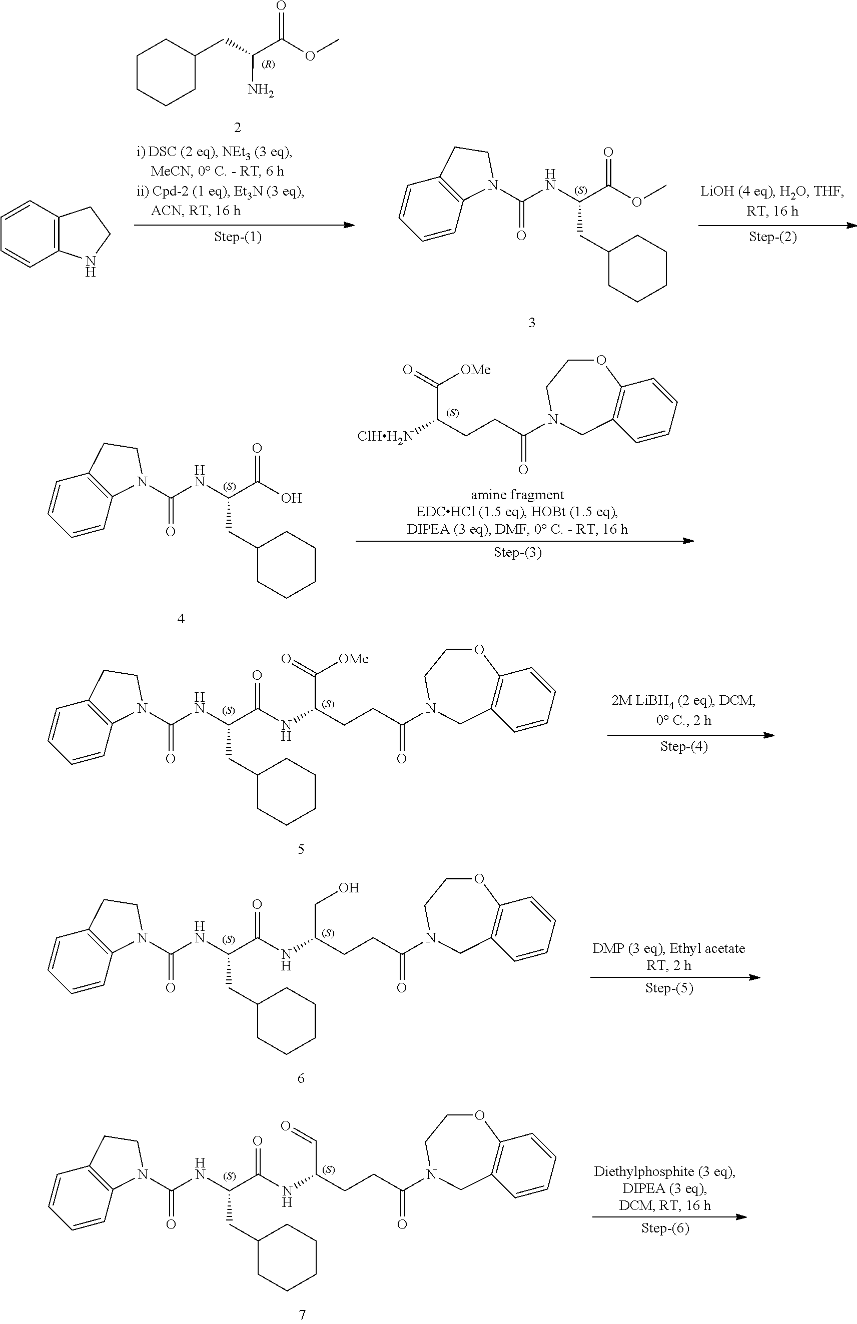

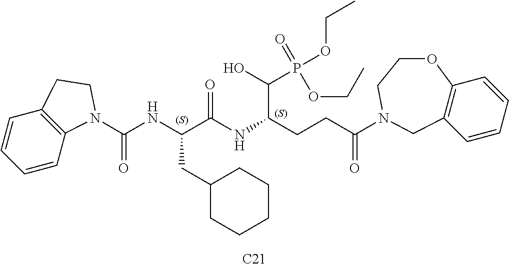

### Methyl (S)-3-cyclohexyl-2-(indoline-1-carboxamido)propanoate (3)

To a stirred solution of indoline (1) (3 g, 25.21 mmol) in ACN (30 mL) was added N,N′ disuccinimidyl carbonate (12.9 g, 50.42 mmol), followed by triethylamine (4.7 mL, 0.327 mmol) at room temperature and stirred for 3 h. The progress of the reaction was monitored by TLC. The reaction mass was used directly in the subsequent reaction.

In another RB flask, methyl (S)-2-amino-3-cyclohexylpropanoate (2) (4 g, 21.73 mmol) was taken in ACN (20 mL), and treated with triethylamine (9.1 mL, 65.21 mmol). The resulting reaction mixture was stirred for 5 min, then added above prepared reaction mass drop-wise and the reaction mixture stirred at room temperature for 16 h. After 16 h, the reaction mixture was quenched with ice water (100 mL) and extracted with ethyl acetate (2×100 mL), combined organic layers were washed with brine solution (50 mL), dried over sodium sulfate and evaporated under reduced pressure. The crude residue was purified by normal phase chromatography to afford methyl (S)-3-cyclohexyl-2-(indoline-1-carboxamido)propanoate (3). TLC system: 50% EtOAc/Pet ether R_{f}: 0.45 LCMS (ESI): m/z 331.34 [M+H]⁺

### (S)-3-cyclohexyl-2-(indoline-1-carboxamido)propanoic acid (4)

To a stirred solution of methyl (S)-3-cyclohexyl-2-(indoline-1-carboxamido)propanoate (3) (2 g, 6.06 mmol) in THF (20 mL) and water (10 mL), was added lithium hydroxide (436 mg, 18.18 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH˜4, and extracted with dichloromethane (2×30 mL), dried over sodium sulfate, concentrated under reduced pressure to afford crude (S)-3-cyclohexyl-2-(indoline-1-carboxamido)propanoic acid (4). TLC system: 5% MeOH/DCM R_{f}: 0.1 LCMS (ESI): m/z 317.49 [M+H]⁺

### Methyl (S)-2-((S)-3-cyclohexyl-2-(indoline-1-carboxamido)propanamido)-5-(2,3-dihydrobenzo[f][1,4]oxazepin-4(5H)-yl)-5-oxopentanoate (5)

At 0° C., to a stirred solution of (S)-3-cyclohexyl-2-(indoline-1-carboxamido)propanoic acid (4) (600 mg, 1.89 mmol) in DMF (20 mL) was added EDC.HCl (543 mg, 2.84 mmol), HOBT (384 mg, 2.84 mmol), DIPEA (1.1 mL, 5.67 mmol) and the reaction mass was stirred for 15 min. After 15 min, added methyl N⁵-methyl-N⁵-phenethyl-L-glutaminate hydrochloride (Amine fragment) (622 mg, 1.89 mmol) and stirred at room temperature for 16 h. The progress of the reaction was monitored by TLC and LCMS. After 16 h, the reaction mixture was quenched with ice water (150 mL) and extracted with ethyl acetate (2×80 mL). Combined organic layers were washed with brine solution (80 mL), the organic layer was dried over sodium sulfate and evaporated under reduced pressure to get crude. Crude residue was purified by normal phase chromatography with eluted 60% EtOAc in Pet ether to afford methyl (S)-2-((S)-3-cyclohexyl-2-(indoline-1-carboxamido)propanamido)-5-(2,3-dihydrobenzo[f][1,4]oxazepin-4(5H)-yl)-5-oxopentanoate (5). TLC system: 5% MeOH/DCM R_{f}: 0.6 LCMS (ESI): m/z 591.17 [M+H]⁺

### N—((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)indoline-1-carboxamide (6)

At 0° C., to a stirred solution of N—((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)indoline-1-carboxamide (5) (100 mg, 0.16 mmol) in DCM (5 mL) was added 2M LiBH₄ in THF (0.14 mL, 0.34 mmol) and the reaction mixture was stirred at RT for 2 h. The progress of the reaction was monitored by TLC and LCMS. The reaction mixture was quenched with water (20 mL) and extracted with DCM (2×20 mL). The combined organic layer was washed with brine solution (20 mL) and the organic layer was dried over Na₂SO₄ and concentrated to get crude compound. Crude compound was purified by normal phase chromatography to afford N—((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)indoline-1-carboxamide (6). TLC system: 5% MeOH/DCM R_{f}: 0.45 LCMS (ESI): m/z 563.48 (M+H)⁺

### N—((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)indoline-1-carboxamide (7)

To a stirred solution of N—((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)indoline-1-carboxamide (6) (150 mg, 0.266 mmol) in ethyl acetate (5 mL) was added Dess-Martin periodinane (334 mg, 0.8 mmol) at 0° C. and stirred at RT for 3 h. The progress of the reaction was monitored by TLC and LCMS. Reaction mixture was diluted with ethyl acetate (50 mL) and washed with sat. NaHCO₃ solution (3×20 mL) followed by sat. Hypo solution (3×20 mL). Organic layer was dried over anhydrous Na₂SO₄, filtered and concentrated to get N—((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)indoline-1-carboxamide (7). TLC system: 5% Methanol in DCM R_{f}: 0.5 LCMS (ESI): m/z 561.45 (M+H)⁺

### Diethyl ((2S)-2-((S)-3-cyclohexyl-2-(indoline-1-carboxamido)propanamido)-5-(2,3-dihydrobenzo[f][1,4]oxazepin-4(5H)-yl)-1-hydroxy-5-oxopentyl)phosphonate (Compound C21)

To a stirred solution of get N—((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)indoline-1-carboxamide (7) (150 mg, 0.26 mmol) was dissolved in DCM (2 mL) was added DIPEA (0.14 mL, 0.8 mmol) and diethyl phosphite (0.11 mL, 0.8 mmol) at 0° C. and stirred at RT for 16 h. Reaction mixture was quenched with ice water (10 mL) extracted with DCM (3×20 mL). Organic layer was dried over anhydrous Na₂SO₄, filtered concentrated to give crude compound. The crude compound was purified by prep HPLC to afford pure diethyl ((2S)-2-((S)-3-cyclohexyl-2-(indoline-1-carboxamido)propanamido)-5-(2,3-dihydrobenzo[f][1,4]oxazepin-4(5H)-yl)-1-hydroxy-5-oxopentyl)phosphonate (Compound C21). TLC system: 80% EtOAc in pet ether Rf: 0.3 LCMS (ESI): m/z 699.58 (M+H)⁺

### Example 13: Synthesis of Compounds C27 and C8

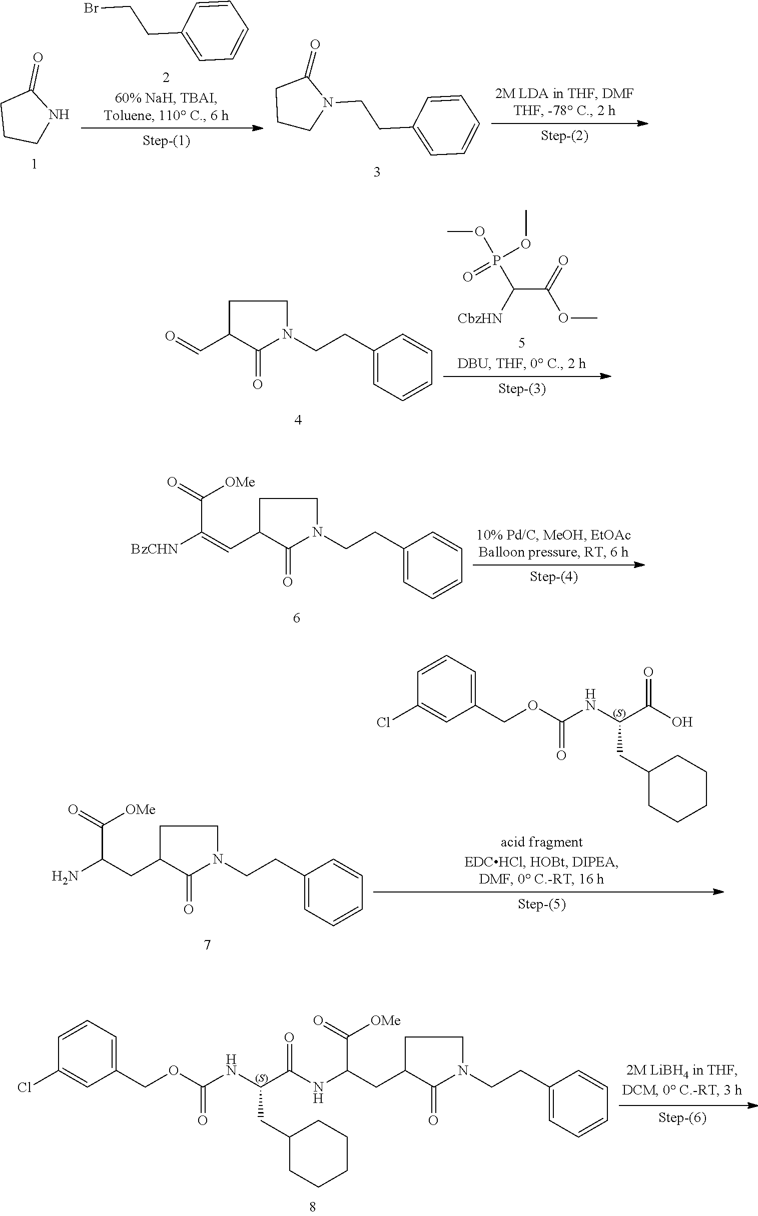

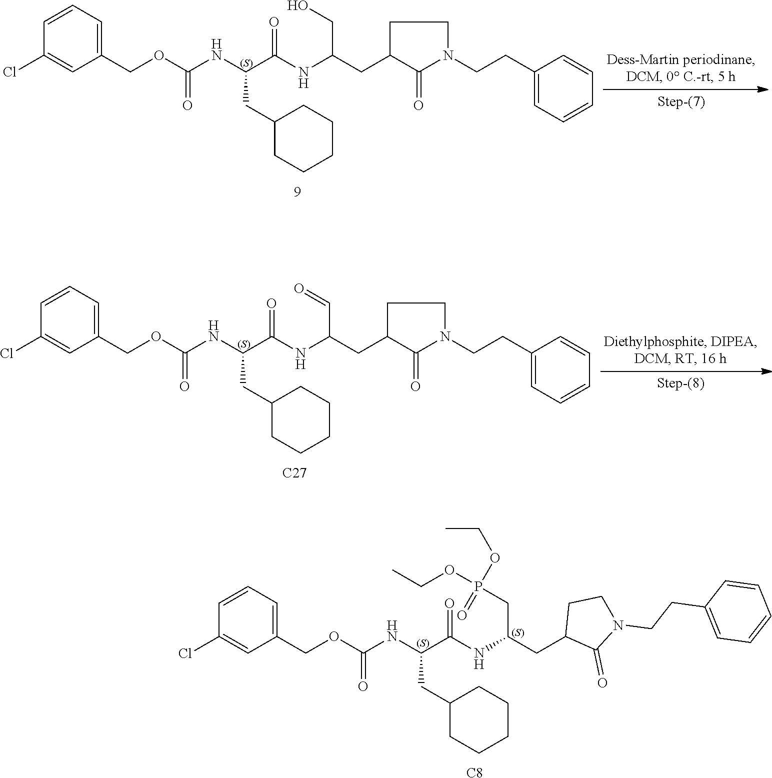

### 1-Phenethylpyrrolidin-2-one (3)

To a stirred solution of pyrrolidin-2-one (1) (10 g, 117.64 mmol) in toluene (150 mL) was added 60% NaH (7.0 g, 176.47 mmol), TBAI (8.68 g, 23.52 mmol) followed by (2-bromoethyl)benzene (2) (21.64 mL, 152.94 mmol) and refluxed for 6 h. Reaction mixture was quenched with ice water (150 mL), extracted with ethyl acetate (2×150 mL), dried over sodium sulfate and evaporated under reduced pressure. The crude residue was purified by silica gel column by eluting with 70% ethyl acetate in hexane to afford 1-phenethylpyrrolidin-2-one (3). TLC system: 80% Ethyl acetate in Hexane Rf: 0.2 LCMS (ESI): m/z 190.29 [M+H]⁺

### 2-Oxo-1-phenethylpyrrolidine-3-carbaldehyde (4)

To a stirred solution of 1-phenethylpyrrolidin-2-one (3) (4.0 g, 21.141 mmol) in THF (60 mL) was added 2M LDA in THF (16 mL, 31.71 mmol) with drop wise at −78° C. Reaction mixture was stirred at −78° C. for 1 h then DMF (2.3 mL, 31.712 mmol) in THF (10 mL) was added and stirred for 2 h at same temperature. The progress of the reaction was monitored by TLC. After consumption of starting material, the reaction mixture was quenched with sat. NH₄Cl solution, extracted with ethyl acetate (2×50 mL), dried over sodium sulfate and evaporated under reduced pressure to afford 2-oxo-1-phenethylpyrrolidine-3-carbaldehyde (4) which was used for next step without any purification. TLC system: 80% Ethyl acetate in Hexane Rf: 0.4 LCMS (ESI): m/z 218.20 [M+H]⁺

### Methyl (E)-2-(((benzyloxy) carbonyl) amino)-3-(2-oxo-1-phenethylpyrrolidin-3-yl) acrylate (6)

To a stirred solution of 2-oxo-1-phenethylpyrrolidine-3-carbaldehyde (4) (4.5 g, crude) in THF (60 mL) was added methyl 2-(((benzyloxy)carbonyl)amino)-2-(dimethoxyphosphoryl) acetate (8.2 g, 24.86 mmol) followed by DBU 4.72 g, 31.07 mmol) at 0° C. and stirred for 2 h. Reaction mixture was diluted with ice water (50 mL), extracted with ethyl acetate (2×40 mL), dried over sodium sulfate and evaporated under reduced pressure. The crude residue was purified by silica gel column by eluting with 25% ethyl acetate in hexane to afford methyl (E)-2-(((benzyloxy)carbonyl)amino)-3-(2-oxo-1-phenethylpyrrolidin-3-yl)acrylate (6). TLC system: 50% Ethyl acetate in Hexane Rf: 0.5 LCMS (ESI): m/z 218.20 [M+H]⁺

### Methyl 2-amino-3-(2-oxo-1-phenethylpyrrolidin-3-yl) propanoate (7)

To a stirred solution of methyl (E)-2-(((benzyloxy) carbonyl) amino)-3-(2-oxo-1-phenethylpyrrolidin-3-yl) acrylate (6) (2.2 g, 5.213 mmol) in methanol (15 mL), ethyl acetate (15 mL) was added 10% Pd/C (500 mg) and stirred for 6 h under H₂ balloon pressure (15 Psi). The progress of the reaction was monitored by TLC and LCMS. After 6 h, the reaction mixture was filtered through celite bed and washed with ethyl acetate (30 mL), filtrate was concentrated under reduced pressure to afford methyl 2-amino-3-(2-oxo-1-phenethylpyrrolidin-3-yl)propanoate (7). TLC system: 50% Ethyl acetate in Hexane Rf: 0.4 LCMS (ESI): m/z 291.28 [M+H]⁺

### Methyl 2-((S)-2-((((3-chlorobenzyl) oxy) carbonyl) amino)-3-cyclohexylpropanamido)-3-(2-oxo-1-phenethylpyrrolidin-3-yl) propanoate (8)

To a stirred solution of (S)-2-((((3-chlorobenzyl)oxy)carbonyl)amino)-3-cyclohexyl propanoic acid (acid fragment) (1.0 g, 2.948 mmol) in DMF (15 mL) was added EDC.HCl (0.84 g, 4.42 mmol), HOBT (0.59 g, 4.42 mmol), DIPEA (1.14 mL, 8.84 mmol) and methyl 2-amino-3-(2-oxo-1-phenethylpyrrolidin-3-yl)propanoate (7) (0.5 g, 1.74 mmol) at 0° C. simultaneously and stirred at room temperature for 16 h. Reaction mixture was diluted with ice water (50 mL), extracted with ethyl acetate (2×40 mL), dried over sodium sulfate and evaporated under reduced pressure. The crude residue was purified by silica gel column by eluting with 2% methanol in dichloromethane to afford methyl 2-((S)-2-((((3-chlorobenzyl)oxy)carbonyl)amino)-3-cyclohexylpropanamido)-3-(2-oxo-1-phenethylpyrrolidin-3-yl)propanoate (8). TLC system: 5% Methanol in dichloromethane Rf: 0.4 LCMS (ESI): m/z 612.47 (M+H)⁺

### 3-Chlorobenzyl ((2S)-3-cyclohexyl-1-((1-hydroxy-3-(2-oxo-1-phenethylpyrrolidin-3-yl) propan-2-yl) amino)-1-oxopropan-2-yl) carbamate (9)

To a stirred solution of methyl 2-((S)-2-((((3-chlorobenzyl)oxy)carbonyl)amino)-3-cyclohexylpropanamido)-3-(2-oxo-1-phenethylpyrrolidin-3-yl)propanoate (8) (400 mg, 0.65 mmol) in DCM (4 mL) was added 2M LiBH₄ in THF (0.65 mL, 1.30 mmol) at 0° C. and the reaction mixture stirred at same temperature for 2 h. The progress of the reaction was monitored by TLC and LCMS. Then reaction mixture was quenched with sat. Ammonium chloride solution (20 mL) and extracted with ethyl acetate (2×20 mL). Organic layer was washed with brine solution (20 mL), dried over Na₂SO₄ and concentrated to afford crude. The crude residue was purified by silica gel column by eluting with 2% methanol in dichloromethane to afford 3-chlorobenzyl ((2S)-3-cyclohexyl-1-((1-hydroxy-3-(2-oxo-1-phenethylpyrrolidin-3-yl) propan-2-yl) amino)-1-oxopropan-2-yl) carbamate (9). TLC system: 5% Methanol in dichloromethane Rf: 0.2 LCMS (ESI): m/z 584.45 (M+H)⁺

### 3-Chlorobenzyl ((2S)-3-cyclohexyl-1-oxo-1-((1-oxo-3-(2-oxo-1-phenethylpyrrolidin-3-yl) propan-2-yl) amino) propan-2-yl) carbamate (Compound C27)

To a stirred solution of 3-chlorobenzyl ((2S)-3-cyclohexyl-1-((1-hydroxy-3-(2-oxo-1-phenethylpyrrolidin-3-yl) propan-2-yl) amino)-1-oxopropan-2-yl) carbamate (9) (130 mg, 0.22 mmol) in dichloromethane (5 mL) was added Dess-Martin periodinane (188 mg, 0.44 mmol) at 0° C. and stirred at RT for 5 h. The progress of the reaction was monitored by TLC and LCMS. Reaction mixture was diluted with dichloromethane (15 mL) and washed with sat. Hypo solution (3×20 mL), sat. NaHCO₃ solution (3×20 mL). Organic layer was dried over anhydrous Na₂SO₄, filtered and concentrated to get crude product, this crude was purified by prep HPLC to afford 3-chlorobenzyl ((2S)-3-cyclohexyl-1-oxo-1-((1-oxo-3-(2-oxo-1-phenethylpyrrolidin-3-yl) propan-2-yl) amino) propan-2-yl) carbamate (Compound C27). TLC system: 10% Methanol in DCM Rf: 0.3 LCMS (ESI): m/z 582.29 (M+H)⁺

### 3-Chlorobenzyl ((2S)-3-cyclohexyl-1-((1-(diethoxyphosphaneyl)-1-hydroxy-3-(2-oxo-1-phenethylpyrrolidin-3-yl) propan-2-yl) amino)-1-oxopropan-2-yl) carbamate (Compound C8)

To a stirred solution of crude 3-chlorobenzyl ((2S)-3-cyclohexyl-1-oxo-1-((1-oxo-3-(2-oxo-1-phenethylpyrrolidin-3-yl) propan-2-yl) amino) propan-2-yl) carbamate (Compound C27) (220 mg, 0.378 mmol) in DCM (5 mL) was added DIPEA (0.2 mL, 1.13 mmol) and diethylphosphate (0.2 mL, 1.13 mmol) at 0° C. and stirred at RT for 16 h. The progress of the reaction was monitored by TLC and LCMS. Reaction mixture was quenched with ice water (15 mL) extracted with DCM (3×15 mL). Organic layer was dried over anhydrous Na₂SO₄, filtered and purified by prep HPLC afforded 3-chlorobenzyl ((2S)-3-cyclohexyl-1-(((2S)-1-(diethoxyphosphoryl)-1-hydroxy-3-(2-oxo-1-phenethylpyrrolidin-3-yl)propan-2-yl)amino)-1-oxopropan-2-yl) carbamate (Compound C8). TLC system: 10% Methanol in DCM Rf: 0.5 LCMS (ESI): m/z 720.58 (M+H)⁺

### Example 14: Synthesis of Compounds C30 and C28

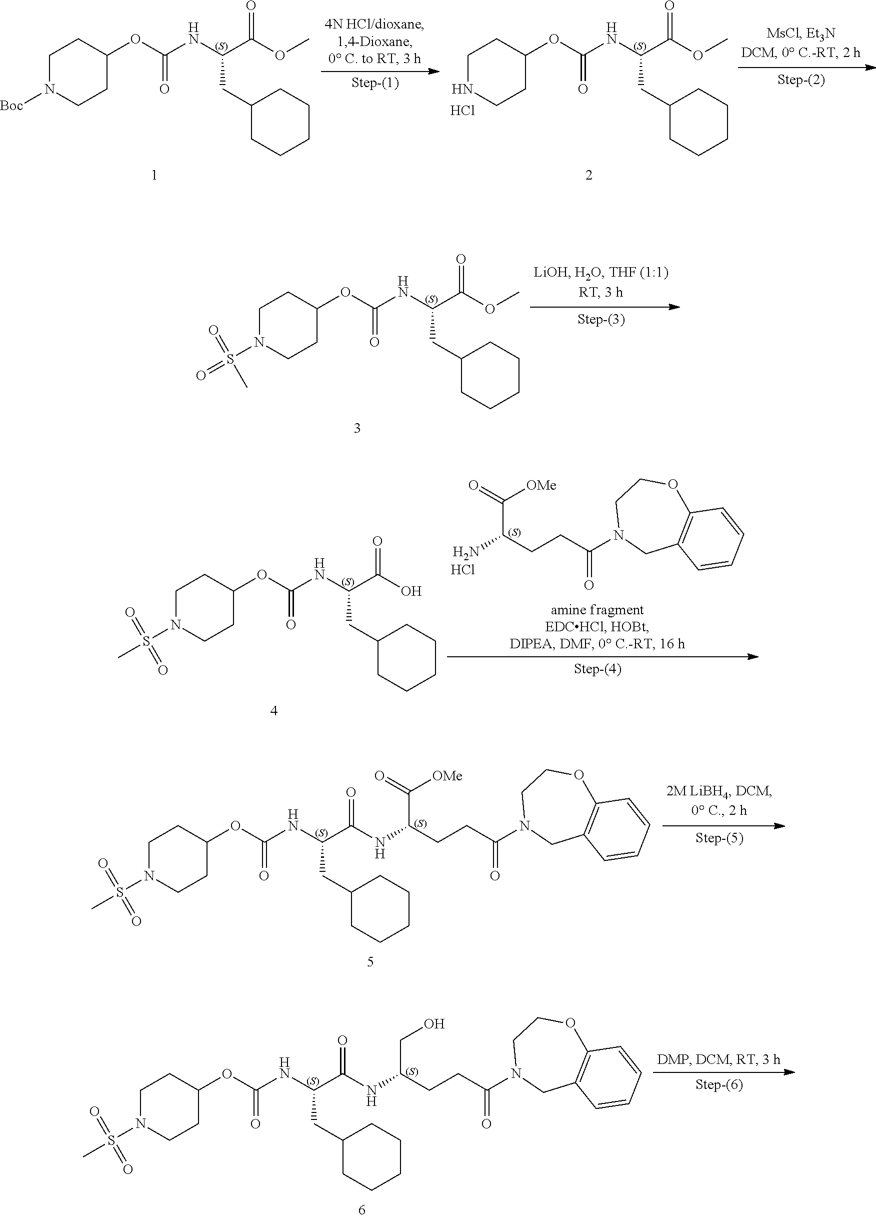

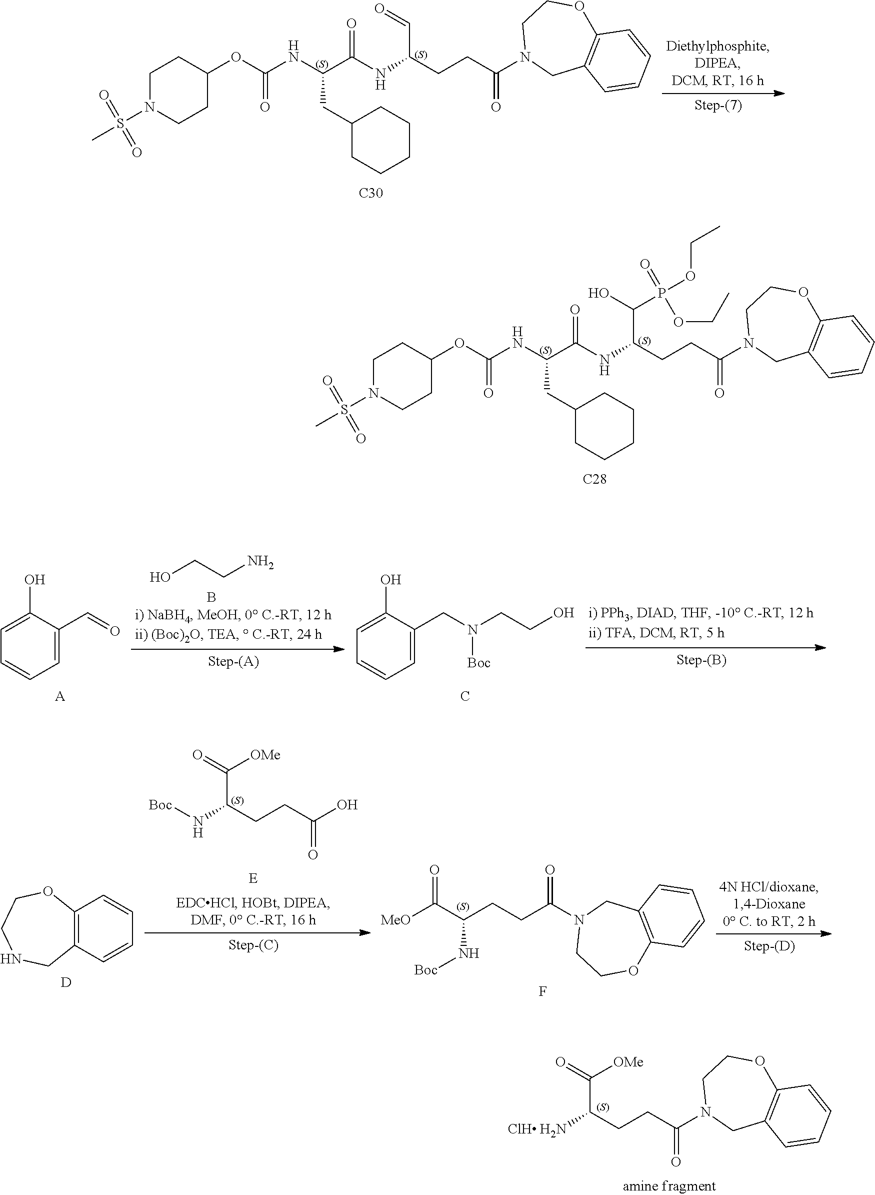

### Tert-butyl (2-hydroxybenzyl) (2-hydroxyethyl) carbamate (C)

To a stirred solution of 2-hydroxybenzaldehyde (A) (25 g, 204.91 mmol) in methanol (50 mL) was added 2-aminoethan-1-ol (12.49 mL, 204.91 mmol) at RT and stirred for 6 h then NaBH₄ (3.89 g, 102.45 mmol) was added at 0° C. and stirred for 6 h. Reaction mixture was cooled to 0° C. and added triethylamine (33.2 mL, 245.89 mmol), (Boc)₂O (49.13 g, 225.40 mmol) and allowed to RT for 24 h. The progress of the reaction was monitored by TLC and LCMS. The reaction mixture was evaporated under reduced pressure and acidified up to pH˜4 with 2N HCl, solids were filtered and washed with water (200 mL), dried under vacuum to afforded Tert-butyl (2-hydroxybenzyl)(2-hydroxyethyl)carbamate (C) TLC system: 30% Ethyl acetate in Pet ether Rf: 0.2 LCMS (ESI): m/z=290.27 [M+Na]⁺

### 2,3,4,5-Tetrahydrobenzo[f][1, 4]oxazepine (C)

To a stirred solution of DIAD (36.72 mL, 187.26 mmol), triphenyl phosphine (49.1 g, 187.26 mmol) in THF (250 mL) was added slowly Tert-butyl (2-hydroxybenzyl) (2-hydroxyethyl) carbamate (C) (25 g, 93.63 mmol) in THF (100 mL) at −10° C. and allowed to RT for 16 h. The progress of the reaction was monitored by TLC. The reaction mixture was evaporated under reduced pressure and dissolved in dichloromethane (200 mL) and added TFA (175 mL) at 0° C. and stirred at RT for 5 h. The progress of the reaction was monitored by TLC, Reaction mixture was evaporated under reduced pressure to afford crude, this crude was diluted with water and washed with diethyl ether (2×100 mL) then aq layer was basified pH˜12 with 10% NaOH and extracted with DCM (2×150 mL), dried over sodium sulfate, concentrated under reduced pressure to afford 2,3,4,5-tetrahydrobenzo[f][1,4]oxazepine (D). TLC system: 80% Ethyl acetate in Pet ether Rf: 0.1 LCMS (ESI): m/z=150.12 [M+H]⁺

### Methyl (S)-2-((tert-butoxycarbonyl) amino)-5-(2,3-dihydrobenzo[f][1, 4]oxazepin-4(5H)-yl)-5-oxopentanoate (F)

To a stirred solution of (S)-4-((tert-butoxycarbonyl)amino)-5-methoxy-5-oxopentanoic acid (E) (10.0 g, 38.31 mmol) in DMF (100 mL) added EDC.HCl (10.9 g, 57.47 mmol), HOBT (7.7 g, 57.47 mmol), DIPEA (20.0 mL, 114.93 mmol) and 2,3,4,5-tetrahydrobenzo[f] [1,4]oxazepine (D) (6.2 g, 42.14 mmol) at 0° C. simultaneously and stirred at room temperature for 16 h. The progress of the reaction was monitored by TLC and LCMS. After 16 h, reaction mixture was quenched with ice water (200 mL) and extracted with ethyl acetate (2×100 mL), dried over Na₂SO₄ and concentrated to get crude compound, this crude was purified by normal phase chromatography with eluted 40% ethyl acetate in hexane to afford methyl (S)-2-((tert-butoxycarbonyl)amino)-5-(2,3-dihydrobenzo[f][1,4]oxazepin-4(5H)-yl)-5-oxopentanoate (F). TLC system: 50% Ethyl acetate in Pet ether Rf: 0.3 LCMS (ESI): m/z=393.38 [M+H]⁺

### Methyl (S)-2-amino-5-(2,3-dihydrobenzo[f][1, 4] oxazepin-4(5H)-yl)-5-oxopentanoate hydrochloride (amine fragment)

To a stirred solution of methyl (S)-2-((tert-butoxycarbonyl)amino)-5-(2,3-dihydrobenzo[f] [1,4]oxazepin-4(5H)-yl)-5-oxopentanoate (F) (5.0 g, 12.75 mmol) in 1,4-dioxane (20 mL) was added 4N HCl in dioxane (20 mL) with drop wise at 0° C. Reaction mixture was stirred at RT for 2 h. The progress of the reaction was monitored by TLC. After consumption of starting material, the reaction mixture was evaporated under reduced pressure to obtained crude compound, the resulting crude triturated with diethyl ether to afford pure methyl(S)-2-amino-5-(2,3-dihydrobenzo[f][1,4]oxazepin-4(5H)-yl)-5-oxopentanoate hydrochloride (amine fragment). TLC system: 5% Methanol in dichloromethane Rf: 0.1 LCMS (ESI): m/z=293.1 [M+H]⁺

### Methyl (S)-3-cyclohexyl-2-(((piperidin-4-yloxy) carbonyl) amino) propanoate hydrogen chloride (2)

To a stirred solution of tert-butyl (S)-4-(((3-cyclohexyl-1-methoxy-1-oxopropan-2-yl) carbamoyl) oxy) piperidine-1-carboxylate (1) (4 g, 9.70 mmol) in 1,4-dioxane (40 mL) was added 4N HCl in dioxane (40 mL) with drop wise at 0° C. Reaction mixture was stirred at RT for 3 h. The progress of the reaction was monitored by TLC. After consumption of starting material, the reaction mixture was evaporated under reduced pressure to obtained crude compound, the resulting crude triturated with diethyl ether to afford methyl (S)-3-cyclohexyl-2-(((piperidin-4-yloxy)carbonyl) amino)propanoate hydrochloride (2). TLC system: 50% Ethyl acetate in Pet ether Rf: 0.1 LCMS (ESI): m/z 313.33[M+H]⁺

### Methyl (S)-3-cyclohexyl-2-((((1-(methylsulfonyl) piperidin-4-yl) oxy) carbonyl) amino) propanoate (3)

To a stirred solution of methyl (S)-3-cyclohexyl-2-(((piperidin-4-yloxy)carbonyl)amino)propanoate (2) (3.3 g, 10.57 mmol) in DCM (40 mL) was added triethylamine (4.5 mL) followed by mesylchloride (1 mL, 12.69 mmol) at 0° C. and stirred for 2 h. The progress of the reaction was monitored by TLC and LCMS. Reaction mixture was diluted with DCM and washed with water (2×30 mL), dried over sodium sulfate, concentrated under reduced pressure to afford methyl (S)-3-cyclohexyl-2-((((1-(methylsulfonyl)piperidin-4-yl)oxy)carbonyl)amino)propanoate (3). TLC system: 70% Ethyl acetate in Pet ether Rf: 0.3 LCMS (ESI): m/z=391.2 [M+H]⁺

### (S)-3-cyclohexyl-2-((((1-(methylsulfonyl) piperidin-4-yl) oxy) carbonyl) amino) propanoic acid (4)

To a stirred solution of methyl (S)-3-cyclohexyl-2-((((1-(methylsulfonyl)piperidin-4-yl)oxy)carbonyl)amino)propanoate (3) (2.0 g, 5.12 mmol) in THF (15 mL), water (15 mL) was added lithium hydroxide (644 mg, 15.38 mmol) at 0° C. and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×30 mL), dried over sodium sulfate, concentrated under reduced pressure to afford (S)-3-cyclohexyl-2-((((1-(methylsulfonyl) piperidin-4-yl)oxy)carbonyl)amino)propanoic acid (4). TLC system: 5% Methanol in DCM R_{f}: 0.2 LCMS (ESI): m/z 377.52 [M+H]⁺

### Methyl (S)-2-((S)-3-cyclohexyl-2-((((1-(methylsulfonyl) piperidin-4-yl) oxy) carbonyl) amino) propanamido)-5-(2,3-dihydrobenzo[f] [1, 4] oxazepin-4(5H)-yl)-5-oxopentanoate (5)

To a stirred solution of (S)-3-cyclohexyl-2-((((1-(methylsulfonyl)piperidin-4-yl)oxy)carbonyl)amino)propanoic acid (4) (1.0 g, 2.65 mmol) DMF (15 mL) added EDC.HCl (764 mg, 3.98 mmol), HOBT (538 mg, 3.98 mmol), DIPEA (1.4 mL, 7.97 mmol) and methyl (S)-2-amino-5-(2,3-dihydrobenzo[f][1,4]oxazepin-4(5H)-yl)-5-oxopentanoatehydrochloride (amine fragment) (0.93 g, 3.19 mmol) at 0° C. simultaneously and stirred at room temperature for 16 h. The progress of the reaction was monitored by TLC and LCMS. After 16 h, reaction mixture was quenched with ice water (50 mL), obtained solids were filtered and washed with excess water then dried under vacuum to afford methyl (S)-2-((S)-3-cyclohexyl-2-((((1-(methylsulfonyl)piperidin-4-yl)oxy)carbonyl)amino)propanamido)-5-(2,3-dihydrobenzo[f] [1,4]oxazepin-4(5H)-yl)-5-oxopentanoate (5). TLC system: 5% Methanol in DCM R_{f}: 0.4 LCMS (ESI): m/z 651.25 [M+H]⁺

### 1-(Methylsulfonyl) piperidin-4-yl ((S)-3-cyclohexyl-1-(((S)-5-(2,3-dihydrobenzo[f] [1,4]oxazepin-4(5H)-yl)-1-hydroxy-5-oxopentan-2-yl)amino)-1-oxopropan-2-yl)carbamate (6)

To a stirred solution of methyl (S)-2-((S)-3-cyclohexyl-2-((((1-(methylsulfonyl)piperidin-4-yl)oxy)carbonyl)amino)propanamido)-5-(2,3-dihydrobenzo[f] [1,4]oxazepin-4(5H)-yl)-5-oxopentanoate (5) (0.5 g, 0.76 mmol) in DCM (15 mL) was added 2M LiBH₄ in THF (0.76 mL, 1.53 mmol) at 0° C. and the reaction mixture stirred for 2 h at 0° C. The progress of the reaction was monitored by TLC and LCMS. Then reaction mixture was quenched with sat. NH₄Cl solution (30 mL) and extracted with ethyl acetate (2×30 mL). Organic layer was washed with brine solution (30 mL), dried over Na₂SO₄ and concentrated to get crude compound. It was triturated with diethyl ether to afford 1-(methylsulfonyl)piperidin-4-yl ((S)-3-cyclohexyl-1-(((S)-5-(2,3-dihydrobenzo[f] [1,4]oxazepin-4(5H)-yl)-1-hydroxy-5-oxopentan-2-yl)amino)-1-oxopropan-2-yl)carbamate (6). TLC system: 5% Methanol in DCM Rf: 0.2 LCMS (ESI): m/z 623.40 (M+H)⁺

### 1-(Methylsulfonyl) piperidin-4-yl((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C30)

To a stirred solution of 1-(methylsulfonyl) piperidin-4-yl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl) carbamate (6) (220 mg, 0.35 mmol) was dissolved in dichloromethane (5 mL) was added Dess-Martin periodinane (449 mg, 1.06 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude product, this residue was purified by prep HPLC to afford 1-(methylsulfonyl)piperidin-4-yl ((S)-3-cyclohexyl-1-(((S)-5-(2,3-dihydrobenzo[f] [1,4]oxazepin-4(5H)-yl)-1,5-dioxopentan-2-yl)amino)-1-oxopropan-2-yl) carbamate Compound C30). TLC system: 5% Methanol in DCM R_{f}: 0.4 LCMS (ESI): m/z 621.44 [M+H]⁺

### 1-(Methylsulfonyl) piperidin-4-yl((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (Compound C28)

To a stirred solution of 1-(methylsulfonyl)piperidin-4-yl((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C30) (220 mg crude, 0.35 mmol) in DCM (10 mL) added DIPEA (0.19 mL, 1.06 mmol) followed by added diethyl phosphite (0.15 mL, 1.06 mmol) and the reaction mixture stirred at RT for 16 h. The progress of the reaction was monitored by TLC and LCMS. After 16 h, the reaction mixture quenched with Sat. ammonium chloride (20 mL) and extracted with DCM (2×20 mL). Combined organic layer was dried over anhydrous Na₂SO₄, and evaporated to afford crude residue. It was purified by prep HPLC to afford 1-(methylsulfonyl)piperidin-4-yl((2S)-3-cyclohexyl-1-(((2S)-1-(diethoxyphosphoryl)-5-(2,3-dihydrobenzo[f] [1,4]oxazepin-4(5H)-yl)-1-hydroxy-5-oxopentan-2-yl)amino)-1-oxopropan-2-yl)carbamate (Compound C28). TLC system: 5% Methanol in DCM R_{f}: 0.4 LCMS (ESI): m/z 759.50 [M+H]⁺

### Example 23: Synthesis of Compound C10

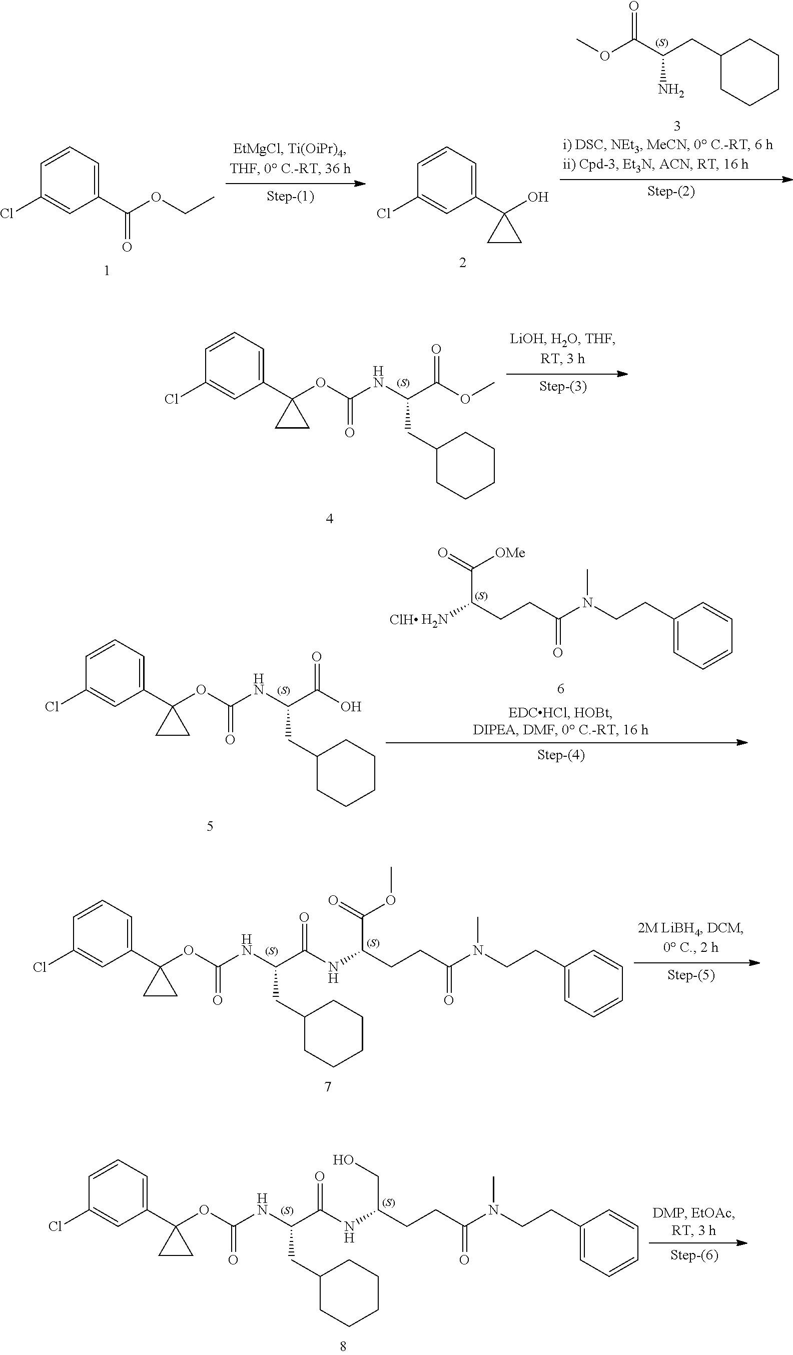

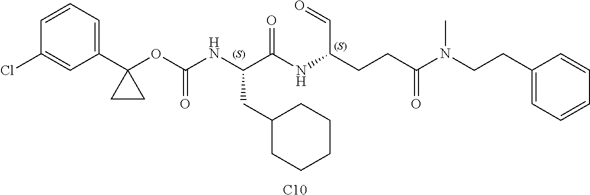

### 1-(3-Chlorophenyl) cyclopropan-1-ol (2)

To a stirred solution of 2-phenylacetonitrile (1) (5 g, 29.41 mmol) in THF (60 mL) were added Titanium isopropoxide (11.69 g, 41.17 mmol) and followed by added 2M Ethyl magnesium chloride in THF (37 mL, 73.52 mmol) slowly drop wise for 30 min at 0° C. Then the reaction mixture stirred at RT for 36 h. The progress of the reaction was monitored by TLC and LCMS. The reaction mixture was quenched with sat ammonium chloride solution (50 mL), extracted with ethyl acetate (3×40 mL), washed with brine solution (100 mL), dried over sodium sulfate, filtered and evaporated under reduced pressure. The crude residue was purified by combi-flash compound eluted at 15% Ethyl acetate in pet ether to afford 1-(3-chlorophenyl) cyclopropan-1-ol (2). TLC system: 20% Ethyl acetate in Pet ether Rf: 0.3 LCMS (ESI): m/z 151.18 [M−OH]⁺

### Methyl (S)-2-(((1-(3-chlorophenyl) cyclopropoxy) carbonyl) amino)-3-cyclohexylpropanoate (4)

To a stirred solution of 1-(3-chlorophenyl)cyclopropan-1-ol (2) (1.4 g, 8.33 mmol) in ACN (20 mL) was added N,N′ disuccinimidyl carbonate (3.19 g, 12.49 mmol), followed by triethylamine (2.8 mL, 24.99 mmol) at room temperature and stirred for 6 h. The progress of the reaction was monitored by TLC. The reaction mass was used directly in the subsequent reaction.

In another RB flask, methyl (S)-2-amino-3-cyclohexylpropanoate (3) (2.70 g, 12.82 mmol) was taken in ACN (20 mL), and treated with triethylamine (3.5 mL, 24.27 mmol). The resulting reaction mixture was stirred for 5 min, then added above prepared reaction mass drop-wise and the reaction mixture stirred at room temperature for 16 h. After 16 h, the reaction mixture was quenched with ice water (100 mL) and extracted with ethyl acetate (2×50 mL), combined organic layers were washed with brine solution (50 mL), dried over sodium sulfate and evaporated under reduced pressure. The crude residue was purified by normal phase chromatography to afford methyl (S)-2-(((1-(3-chlorophenyl) cyclopropoxy) carbonyl) amino)-3-cyclohexylpropanoate (4). TLC system: 20% Ethyl acetate in Pet ether Rf: 0.6 LCMS (ESI): m/z=380.44 [M+H]⁺

### (S)-2-(((1-(3-chlorophenyl) cyclopropoxy) carbonyl) amino)-3-cyclohexylpropanoic acid (5)

To a stirred solution of methyl (S)-2-(((1-(3-chlorophenyl)cyclo propoxy) carbonyl)amino)-3-cyclohexyl propanoate (4) (1.3 g, 3.43 mmol) in THF (20 mL), water (10 mL) was added lithium hydroxide (246 mg, 10.29 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH˜3 and extracted with ethyl acetate (2×30 mL), dried over sodium sulfate, concentrated under reduced pressure to afford crude (S)-2-(((1-(3-chlorophenyl) cyclopropoxy) carbonyl) amino)-3-cyclohexylpropanoic acid (5). TLC system: 5% Methanol in DCM R_{f}: 0.1 LCMS (ESI): m/z 366.43 [M+H]⁺

### Methyl N2-((S)-2-(((1-(3-chlorophenyl) cyclopropoxy) carbonyl) amino)-3-cyclohexylpropanoyl)-N5-methyl-N5-phenethyl-L-glutaminate (7)

To a stirred solution of (S)-2-(((1-(3-chlorophenyl) cyclopropoxy) carbonyl)amino)-3-cyclohexylpropanoic acid (5) (1.0 g, 2.73 mmol) DMF (10 mL) added EDC.HCl (0.78 g, 4.10 mmol), HOBT (0.55 g, 4.10 mmol), DIPEA (1.5 mL, 8.21 mmol) and methyl N5-methyl-N5-phenethyl-L-glutaminate hydrochloride (6) (1.03 g, 3.28 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×50 mL). The combined organic layer was dried over sodium sulfate, filtered and evaporated under reduced pressure. The crude residue was purified by combi-flash, compound eluted at 60% Ethyl acetate in pet ether to afford methyl N2-((S)-2-(((1-(3-chlorophenyl)cyclopropoxy)carbonyl)amino)-3-cyclohexyl propanoyl)-N5-methyl-N5-phenethyl-L-glutaminate (7). TLC system: 80% Ethyl acetate in Pet ether Rf: 0.5 LCMS (ESI): m/z 627.47 [M+H]⁺

### 1-(3-Chlorophenyl) cyclopropyl ((S)-3-cyclohexyl-1-(((S)-1-hydroxy-5-(methyl (phenethyl) amino)-5-oxopentan-2-yl) amino)-1-oxopropan-2-yl) carbamate (8)

To a stirred solution of methyl N2-((S)-2-(((1-(3-chlorophenyl) cyclopropoxy)carbonyl)amino)-3-cyclohexylpropanoyl)-N5-methyl-N5-phenethyl-L-glutaminate (7) (1 g, 1.60 mmol) in THF (10 mL) was added 2M LiBH₄ in THF (2.4 mL, 4.80 mmol) at 0° C. and the reaction mixture stirred for 2 h at 0° C. The progress of the reaction was monitored by TLC and LCMS. Then reaction mixture was quenched with water (10 mL) and extracted with ethyl acetate (2×30 mL). Organic layer was washed with brine solution (20 mL), dried over Na₂SO₄ and concentrated to get crude compound. It was triturated with diethyl ether to afford 1-(3-chlorophenyl) cyclopropyl ((S)-3-cyclohexyl-1-(((S)-1-hydroxy-5-(methyl (phenethyl) amino)-5-oxopentan-2-yl) amino)-1-oxopropan-2-yl) carbamate (8). TLC system: 100% Ethyl acetate Rf: 0.2 LCMS (ESI): m/z 598.60 (M+H)⁺

### 1-(3-Chlorophenyl) cyclopropyl ((S)-3-cyclohexyl-1-(((S)-5-(methyl (phenethyl) amino)-1,5-dioxopentan-2-yl) amino)-1-oxopropan-2-yl) carbamate (Compound 010)

To a stirred solution of 1-(3-chlorophenyl) cyclopropyl ((S)-3-cyclohexyl-1-(((S)-1-hydroxy-5-(methyl(phenethyl)amino)-5-oxopentan-2-yl)amino)-1-oxopropan-2-yl) carbamate (8) (150 mg, 0.25 mmol) was dissolved in ethyl acetate (10 mL) was added Dess-Martin periodinane (319 mg, 0.75 mmol) at 0° C. and stirred at RT for 3 h. Reaction mixture was diluted with ethyl acetate (10 mL) and washed with sat. Hypo solution (3×10 mL), sat. NaHCO₃ solution (3×20 mL). Organic layer was dried over anhydrous Na₂SO₄, filtered and concentrated to afford crude product, this residue was purified by normal phase chromatography by eluting 3% methanol in dichloromethane to afford 1-(3-chlorophenyl)cyclopropyl((S)-3-cyclohexyl-1-(((S)-5-methyl(phenethyl)amino)-1,5-dioxopentan-2-yl) amino)-1-oxopropan-2-yl) carbamate (Compound C10). TLC system: 5% Methanol in DCM Rf: 0.4 LCMS (ESI): m/z 596.43 (M+H)⁺

### Example 27: Synthesis of Compound C4

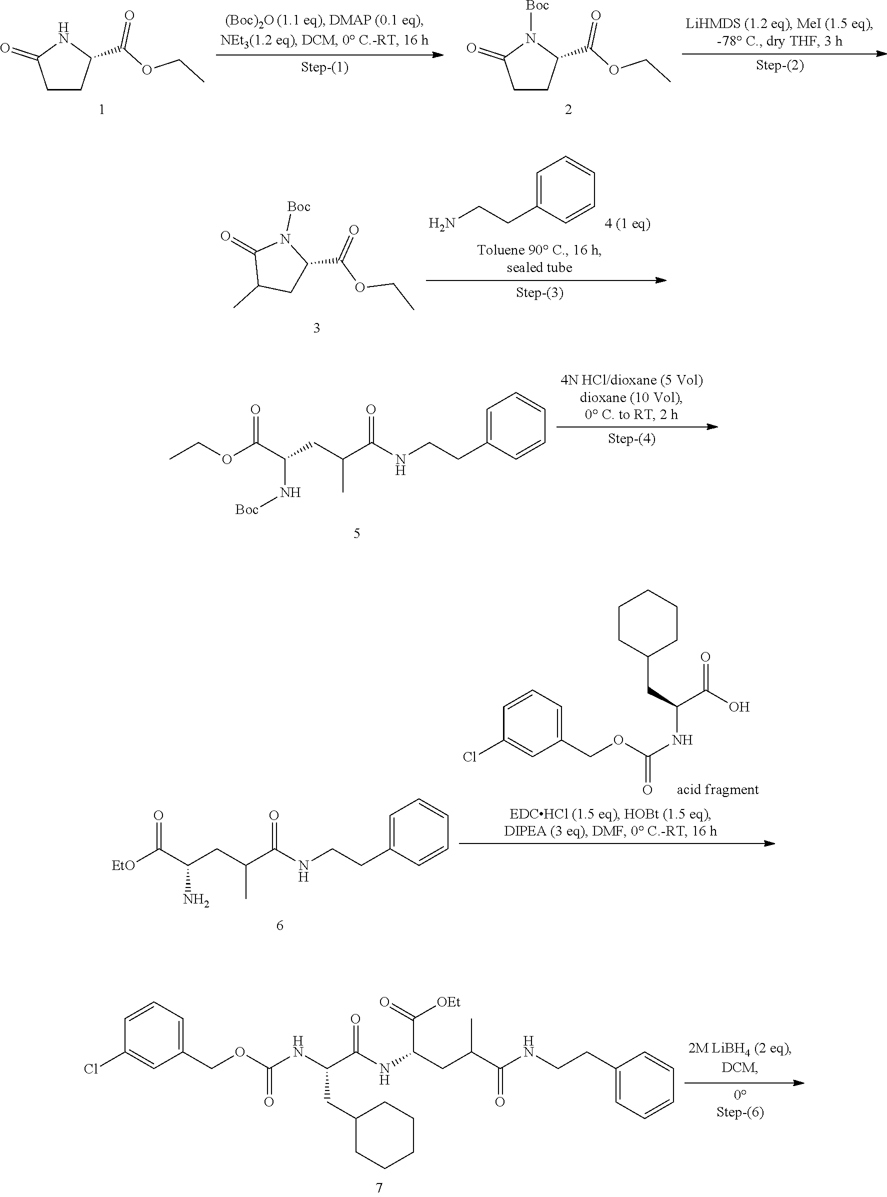

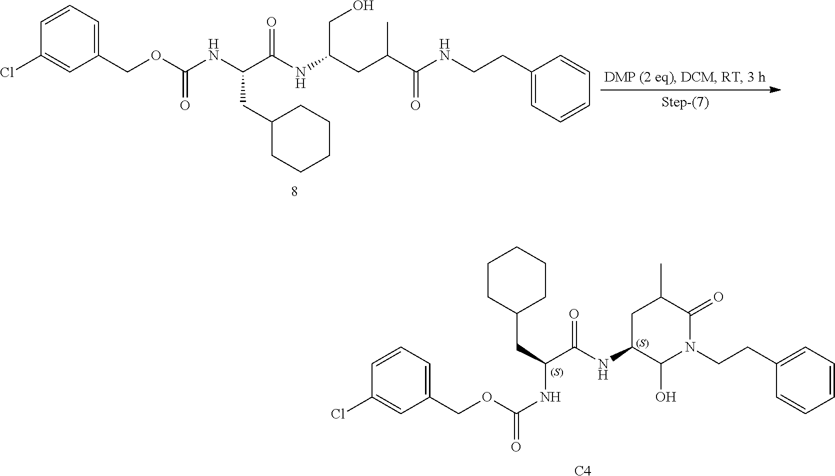

### 1-(tert-butyl) 2-ethyl (S)-5-oxopyrrolidine-1,2-dicarboxylate (1)

To a stirred solution of ethyl (S)-5-oxopyrrolidine-2-carboxylate (1) (20.0 g, 127.38 mmol) in DCM (200 mL) was added Triethylamine (22.02 mL, 152.86 mmol), Boc anhydride (30.54 mL, 140.12 mmol) and DMAP (1.5 g, 12.73 mmol) at 0° C. and reaction mixture was stirred at room temperature for 16 h. The progress of the reaction was monitored by TLC, the reaction mixture was quenched with ice water (500 mL) and extracted with Dichloromethane (3×400 mL) dried over sodium sulfate and concentrated under reduced pressure. The crude residue was purified by grace NP, compound eluted with 30% ethyl acetate and pet ether to afford 1-(tert-butyl) 2-ethyl (S)-5-oxopyrrolidine-1,2-dicarboxylate (2) (29.0 g, 112.71 mmol, 88% yield) as an off-white solid. TLC system: 30% Ethyl acetate in pet ether Rf: 0.3 LCMS (ESI): m/z 258.23 (M+H)⁺

### 1-(tert-butyl) 2-ethyl (2S)-4-methyl-5-oxopyrrolidine-1,2-dicarboxylate (2)

To a stirred solution of 1-(tert-butyl) 2-ethyl (S)-5-oxopyrrolidine-1,2-dicarboxylate (2) (6.0 g, 23.34 mmol) in dry THF (600 mL) was added 1M LiHMDS (28 mL, 28.01 mmol) at −78° C. and stirred for 3 h. The progress of the reaction was monitored by TLC, the reaction mixture was quenched saturated ammonium chloride solution (100 mL) and extracted with Ethyl acetate (2×200 mL), the organic layer was dried over sodium sulfate and concentrated under reduced pressure. The crude residue was purified by grace NP, compound eluted with 20% ethyl acetate and pet ether to afford 1-(tert-butyl) 2-ethy(2S)-4-methyl-5-oxopyrrolidine-1,2-dicarboxylate (3) (1.4 g, 5.16 mmol, 22% yield) as a clear gummy liquid. TLC system: 20% Ethyl acetate in pet ether Rf: 0.4 LCMS (ESI): m/z 272.28 (M+H)⁺

### Ethyl (2S)-2-((tert-butoxycarbonyl)amino)-4-methyl-5-oxo-5-(phenethylamino)pentanoate (5)

To a stirred solution of 1-(tert-butyl) 2-ethy(2S)-4-methyl-5-oxopyrrolidine-1,2-dicarboxylate (3) (1.4 g, 5.16 mmol) in toluene (50 mL) was added 2-phenylethylamine (4) (625 mg, 5.16 mmol), and heated in sealed tube at 90° C. for 6 h. The progress of the reaction was monitored by TLC, reaction mixture was concentrated. The crude residue was purified by grace NP, compound eluted y eluting with 50% ethyl acetate and pet ether to afford Ethyl (2S)-2-((tert-butoxycarbonyl)amino)-4-methyl-5-oxo-5-(phenethylamino)pentanoate (5) (1.0 g, 2.549 mmol, 49% yield) as clear gummy liquid. TLC system: 50% Ethyl acetate in pet ether Rf: 0.5 LCMS (ESI): m/z 393.4 (M+H)⁺

### Ethyl (2S)-2-amino-4-methyl-5-oxo-5-(phenethylamino)pentanoate (6)

To a stirred solution of Ethyl (2S)-2-((tert-butoxycarbonyl)amino)-4-methyl-5-oxo-5-(phenethylamino)pentanoate (5) (1.0 g, 2.98 mmol) in 1,4-dioxane (10 mL) was added 4M HCl in dioxane (10 mL) with drop wise at 0° C. Reaction mixture was stirred at RT for 2 h. The progress of the reaction was monitored by TLC. After consumption of starting material, the reaction mixture was evaporated under reduced pressure to obtain crude compound, the resulting crude triturated with diethyl ether to afford ethyl (2S)-2-amino-4-methyl-5-oxo-5-(phenethylamino)pentanoate (6) (0.720 g, 2.462 mmol, 96% yield) as an off white solid TLC system: 70% Ethyl acetate in pet ether R_{f}: 0.9 LCMS (ESI): m/z 293.28 (M+H)⁺

### Ethyl (2S)-2-((S)-2-((((3-chlorobenzyl)oxy)carbonyl)amino)-3-cyclohexylpropanamido)-4-methyl-5-oxo-5-(phenethylamino)pentanoate (7)

To a stirred solution of (S)-2-((((3-chlorobenzyl)oxy)carbonyl)amino)-3-cyclohexylpropanoic acid (acid fragment) (0.830 g, 2.448 mmol) in DMF (10 mL), added EDC.HCl (0.71 g, 3.672 mmol), HOBt (0.49 g, 3.67 mmol), DIPEA (1.35 mL, 7.345 mmol) and ethyl (2S)-2-amino-4-methyl-5-oxo-5-(phenethylamino)pentanoate (6) (0.714 g, 2.448 mmol) at 0° C. and reaction mixture was stirred at room temperature for 16 h. The progress of the reaction was monitored by TLC and LCMS. After 16 h, the reaction mixture was diluted with ice water (50 mL) and extracted with ethyl acetate (2×50 mL). Organic layer was dried over sodium sulfate and evaporated under reduced pressure. The crude residue was purified by grace NP, compound eluted at 50% methanol in dichloromethane to afford ethyl (2S)-2-((S)-2-((((3-chlorobenzyl)oxy)carbonyl)amino)-3-cyclohexylpropanamido)-4-methyl-5-oxo-5-(phenethylamino)pentanoate (7) (1.3 g, 2.116 mmol, 86% yield) as an off white solid. TLC system: 50% Ethyl acetate in pet ether R_{f}: 0.4 LCMS (ESI): m/z 614.47 (M+H)⁺

### 3-chlorobenzyl ((2S)-3-cyclohexyl-1-(((2S)-1-hydroxy-4-methyl-5-oxo-5-(phenethylamino)pentan-2-yl)amino)-1-oxopropan-2-yl)carbamate (8)

To a stirred solution of ethyl (2S)-2-((S)-2-((((3-chlorobenzyl)oxy)carbonyl)amino)-3-cyclohexylpropanamido)-4-methyl-5-oxo-5-(phenethylamino)pentanoate (7) (0.250 g, 0.407 mmol) in DCM (20 mL) was added 2M LiBH₄ in THF (0.407 mL, 0.814 mmol) at 0° C., reaction mixture stirred for 2 h at 0° C. The progress of the reaction was monitored by TLC and LCMS. Then the reaction mixture was quenched with saturated NH₄Cl solution (20 mL) and extracted with DCM (2×20 mL). Organic layer was washed with brine solution (20 mL), dried over Na₂SO₄ and concentrated to get crude to afford 3-chlorobenzyl ((2S)-3-cyclohexyl-1-(((2S)-1-hydroxy-4-methyl-5-oxo-5-(phenethylamino)pentan-2-yl)amino)-1-oxopropan-2-yl)carbamate (8) (0.200 g, 0.349 mmol, 86% yield) as off white solid. TLC system: 100% Ethyl acetate R_{f}: 0.6 LCMS (ESI): m/z 572.49 (M+H)⁺

### 3-Chlorobenzyl((2S)-3-cyclohexyl-1-(((3S)-2-hydroxy-5-methyl-6-oxo-1-(phenethylpiperidin-3-yl)amino)-1-oxopropan-2-yl)carbamate (Compound C4)

To a stirred solution of 3-chlorobenzyl ((2S)-3-cyclohexyl-1-(((2S)-1-hydroxy-4-methyl-5-oxo-5-(phenethylamino)pentan-2-yl)amino)-1-oxopropan-2-yl)carbamate (8) (200 mg, 0.349 mmol) in DCM (10 mL) was added Dess-Martin periodinane (296.6 mg, 0.699 mmol) at 0° C. and stirred at RT for 3 h. The progress of the reaction was monitored by TLC and LCMS. Reaction mixture was diluted with DCM (15 mL) and washed with sat. Hypo solution (3×20 mL), sat. NaHCO₃ solution (3×20 mL). Organic layer was dried over anhydrous Na₂SO₄, filtered and concentrated to get crude compound. The crude residue was purified by prep. HPLC to afford 3-chlorobenzyl((2S)-3-cyclohexyl-1-(((3S)-2-hydroxy-5-methyl-6-oxo-1-(phenethylpiperidin-3-yl)amino)-1-oxopropan-2-yl)carbamate (Compound C4) (40 mg, 0.0877 mmol, 25% yield) as an off white solid. TLC system: 100% Ethyl acetate R_{f}: 0.5 LCMS (ESI): m/z 552.89 (M−OH)⁺

### Example 28: Synthesis of Compounds C17 and C5

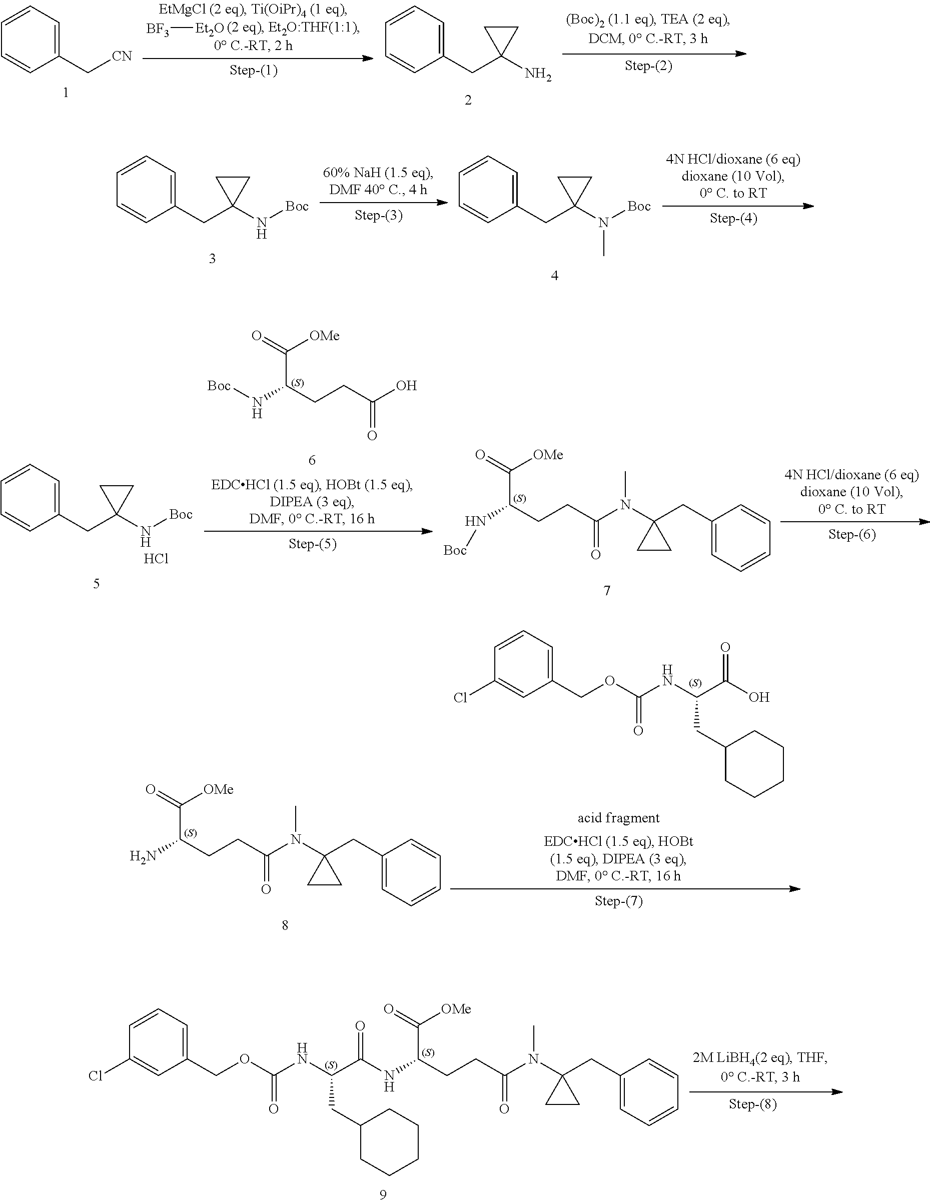

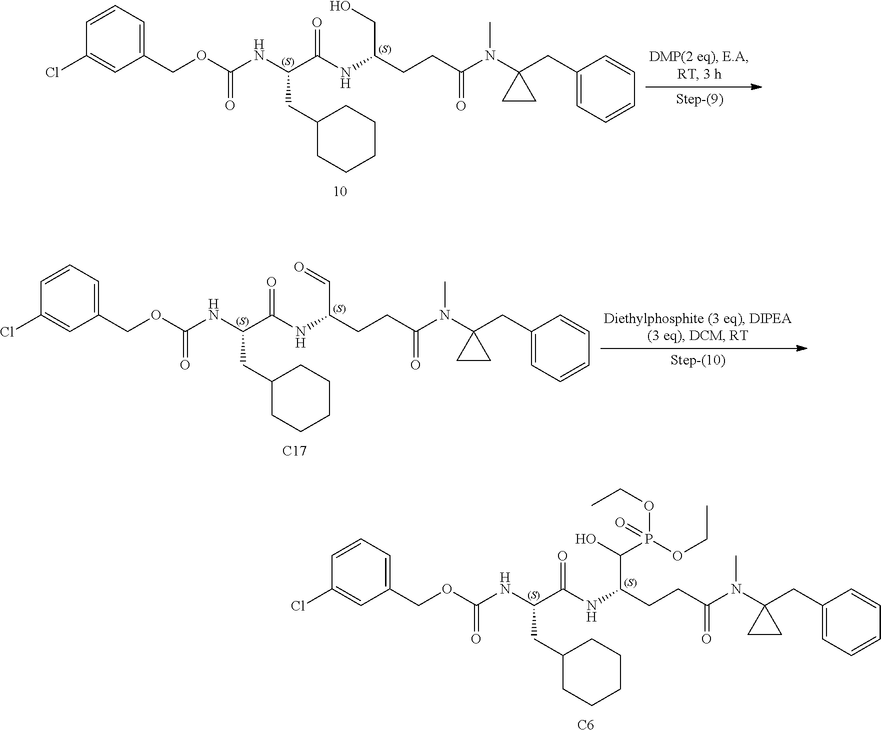

### 1-benzylcyclopropan-1-amine (2)

To a stirred solution of 2-phenylacetonitrile (1) (2 g, 17.094 mmol) in Et₂O:THF (1:1) (20 mL) were added Titanium iso propoxide (5.14 g, 18.119 mmol) and followed by added 2M Ethyl magnesium chloride in THF (17 mL, 34.188 mmol) slowly drop wise for 10 min at 0° C. Then the reaction mixture stirred at RT for 1 h and then added BF₃-Et₂O (4.8 mL, 34.188 mmol) slowly at 0° C. for 15 min (exothermic occurred) and stirred at room temperature for 1 h. The progress of the reaction was monitored by TLC and LCMS. After 1 h, the reaction mixture was poured in 10% NaOH solution (100 mL) white precipitate formed. The reaction mixture filtered through celite bed and washed with ethyl acetate (50 mL) and filtrate washed with brine solution (100 ml) and combined organic layer was dried over sodium sulfate, filtered and evaporated under reduced pressure. The crude residue was purified by combi-flash compound eluted at 70% Ethyl acetate in pet ether to afford 1-benzylcyclopropan-1-amine (2). TLC system: 80% Ethyl acetate in Pet ether Rf: 0.3 LCMS (ESI): m/z 148.11[M+H]⁺

### Tert-butyl (1-benzylcyclopropyl) carbamate (3)

To a stirred solution of 1-benzylcyclopropan-1-amine (2) (4.2 g, 28.5714 mmol) in DCM (50 mL) were added TEA (4.1 mL, 57.142 mmol) and followed by added Boc anhydride (6.8 mL, 31.428 mmol) slowly drop wise for 10 min at 0° C. Then the reaction mixture stirred at RT for 3 h. The progress of the reaction was monitored by TLC and LCMS. After 3 h, the reaction mixture was diluted with ice water (50 mL), extracted with DCM (2×50 mL). The combined organic layer was dried over sodium sulfate, filtered and evaporated under reduced pressure. The crude residue was purified by combi-flash compound eluted at 5% Ethyl acetate in pet ether to afford tert-butyl (1-benzylcyclopropyl)carbamate (3). TLC system: 30% Ethyl acetate in Pet ether Rf: 0.8 LCMS (ESI): m/z 148.11 [M−Boc]⁺

### Tert-butyl (1-benzylcyclopropyl)(methyl)carbamate (4)

To a stirred solution of tert-butyl (1-benzylcyclopropyl) carbamate (3) (2×2.5 g, 10.121 mmol) in DMF (25 mL) in sealed vessel added 60% NaH (607 mg, 15.182 mmol) at 0° C. and stirred for 15 min after added methyl iodide (2.6 mL, 40.485 mmol) slowly drop wise for 10 min at 0° C. Then the reaction mixture heated to 40° C. for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure to afford tert-butyl (1-benzylcyclopropyl)(methyl)carbamate (4). TLC system: 5% Ethyl acetate in Pet ether Rf: 0.6 LCMS (ESI): m/z 162.11 [M−Boc]⁺

### 1-benzyl-N-methylcyclopropan-1-amine hydrochloride (5)

To a stirred solution of tert-butyl (1-benzylcyclopropyl)(methyl)carbamate (4) (1.3 g, 4.9808 mmol in 1,4-dioxane (10 mL) was added 4N HCl in dioxane (20 mL) with drop wise at 0° C. and the reaction mixture was stirred at RT for 3 h. The progress of the reaction was monitored by TLC and LCMS. After consumption of starting material, the reaction mixture was evaporated under reduced pressure to obtained crude compound, the resulting crude triturated with diethyl ether to afford 1-benzyl-N-methylcyclopropan-1-aminehydrochloride (5). TLC system: 5% Methanol in DCM Rf: 0.2 LCMS (ESI): m/z 162.32 [M+H]⁺

### Methyl N5-(1-benzylcyclopropyl)-N2-(tert-butoxycarbonyl)-N5-methyl-L-glutaminate (7)

To a stirred solution of (S)-4-((tert-butoxycarbonyl)amino)-5-methoxy-5-oxopentanoic acid (6) (1 g, 3.831 mmol) in DMF (10 mL) were added EDC.HCl (1.1 g, 5.747 mmol), HOBT (775 mg, 5.747 mmol), DIPEA (2.11 mL, 11.494 mmol) and 1-benzyl-N-methylcyclopropan-1-aminehydrochloride (5) (678 mg, 4.214 mmol) at 0° C. simultaneously and stirred at room temperature for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 40% Ethyl acetate in pet ether to afford methyl N5-(1-benzylcyclopropyl)-N2-(tert-butoxycarbonyl)-N5-methyl-L-glutaminate (7). TLC system: 50% Ethyl acetate in Pet ether Rf: 0.5 LCMS (ESI): m/z 305.19 [M−Boc]⁺

### Methyl N5-(1-benzylcyclopropyl)-N5-methyl-L-glutaminate hydrochloride (8)

To a stirred solution of methyl N5-(1-benzylcyclopropyl)-N2-(tert-butoxycarbonyl)-N5-methyl-L-glutaminate (7) (1.0 g, 2.475 mmol) in 1,4-dioxane (10 mL) was added 4N HCl in dioxane (20 mL) with drop wise at 0° C. and the reaction mixture was stirred at RT for 3 h. The progress of the reaction was monitored by TLC and LCMS. After consumption of starting material, the reaction mixture was evaporated under reduced pressure to obtained crude compound, the resulting crude triturated with diethyl ether to afford methyl N5-(1-benzylcyclopropyl)-N5-methyl-L-glutaminate hydrochloride (8). TLC system: 5% Methanol in DCM Rf: 0.2 LCMS (ESI): m/z 305.26 [M+H]⁺

### Methyl N5-(1-benzylcyclopropyl)-N2-((S)-2-((((3-chlorobenzyl)oxy)carbonyl)amino)-3-cyclohexylpropanoyl)-N5-methyl-L-glutaminate (9)

To a stirred solution of (S)-2-((((3-chlorobenzyl)oxy)carbonyl)amino)-3-cyclohexylpropanoic acid (acid fragment) (1.0 g, 2.949 mmol) DMF (10 mL) added EDC.HCl (845 mg, 4.424 mmol), HOBT (597 mg, 5.899 mmol), DIPEA (1.63 mL, 8.849 mmol) and methyl N5-(1-benzylcyclopropyl)-N5-methyl-L-glutaminate hydrochloride (8) (986 mg, 3.244 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×50 mL). The combined organic layer was dried over sodium sulfate, filtered and evaporated under reduced pressure. The crude residue was purified by combi-flash, compound eluted at 60% Ethyl acetate in pet ether to afford methyl N5-(1-benzylcyclopropyl)-N2-((S)-2-((((3-chlorobenzyl)oxy)carbonyl)amino)-3-cyclohexylpropanoyl)-N5-methyl-L-glutaminate (9). TLC system: 50% Ethyl acetate in Pet ether Rf: 0.5 LCMS (ESI): m/z 626.58 [M+H]⁺

### 3-Chlorobenzyl ((S)-1-(((S)-5-((1-benzylcyclopropyl)(methyl)amino)-1-hydroxy-5-oxopentan-2-yl)amino)-3-cyclohexyl-1-oxopropan-2-yl)carbamate (10)

To a stirred solution of methyl N5-(1-benzylcyclopropyl)-N2-((S)-2-((((3-chlorobenzyl)oxy)carbonyl)amino)-3-cyclohexylpropanoyl)-N5-methyl-L-glutaminate (9) (1 g, 1.6 mmol) in THF (10 mL) was added 2M LiBH₄ in THF (1.6 mL, 3.2 mmol) at 0° C. and the reaction mixture stirred for 2 h at 0° C. The progress of the reaction was monitored by TLC and LCMS. Then reaction mixture was quenched with water (10 mL) and extracted with ethyl acetate (2×30 mL). Organic layer was washed with brine solution (10 mL), dried over Na₂SO₄ and concentrated to get crude compound. It was purified combi-flash, compound eluted at 80% Ethyl acetate in pet ether to afford 3-chlorobenzyl ((S)-1-(((S)-5-((1-benzylcyclopropyl)(methyl)amino)-1-hydroxy-5-oxopentan-2-yl)amino)-3-cyclohexyl-1-oxopropan-2-yl)carbamate (10). TLC system: 100% Ethyl acetate Rf: 0.2 LCMS (ESI): m/z 598.98 (M+H)⁺

### 3-Chlorobenzyl ((S)-1-(((S)-5-((1-benzylcyclopropyl)(methyl)amino)-1,5-dioxopentan-2-yl)amino)-3-cyclohexyl-1-oxopropan-2-yl)carbamate (Compound C17)

To a stirred solution of 3-chlorobenzyl ((S)-1-(((S)-5-((1-benzylcyclopropyl)(methyl)amino)-1-hydroxy-5-oxopentan-2-yl)amino)-3-cyclohexyl-1-oxopropan-2-yl)carbamate (10) (150 mg, 0.2508 mmol) was dissolved in ethyl acetate (10 mL) was added Dess-Martin periodinane (320 mg, 0.752 mmol) at 0° C. and stirred at RT for 3 h. Reaction mixture was diluted with ethyl acetate (10 mL) and washed with sat. Hypo solution (3×10 mL), sat. NaHCO₃ solution (3×20 mL). Organic layer was dried over anhydrous Na₂SO₄, filtered and concentrated to get crude compound. It was purified combi-flash, compound eluted at 20% MeOH in DCM to afford 3-chlorobenzyl ((S)-1-(((S)-5-((1-benzylcyclopropyl)(methyl)amino)-1,5-dioxopentan-2-yl)amino)-3-cyclohexyl-1-oxopropan-2-yl)carbamate (Compound C17). TLC system: 80% Ethyl acetate in pet ether Rf: 0.4 LCMS (ESI): m/z 598.98 (M+H)⁺

### 3-Chlorobenzyl ((2S)-1-(((2S)-5-((1-benzylcyclopropyl)(methyl)amino)-1-(diethoxyphosphoryl)-1-hydroxy-5-oxopentan-2-yl)amino)-3-cyclohexyl-1-oxopropan-2-yl)carbamate (Compound C5)

To a stirred solution of 3-chlorobenzyl ((S)-1-(((S)-5-((1-benzylcyclopropyl)(methyl)amino)-1,5-dioxopentan-2-yl)amino)-3-cyclohexyl-1-oxopropan-2-yl)carbamate (Compound C17) (200 mg, 0.033 mmol) in DCM (10 mL) added DIPEA (0.2 mL, 1.032 mmol) followed by added diethyl phosphite (0.14 mL, 1.032 mmol) and the reaction mixture stirred at RT for 16 h. The progress of the reaction was monitored by TLC and LCMS. After 16 h, the reaction mixture quenched with ammonium chloride (15 mL) and extracted with DCM (2×20 mL). Combined organic layer was dried over anhydrous Na₂SO₄, and evaporated to afford crude residue. It was purified by prep HPLC to afford 3-chlorobenzyl ((2S)-1-(((2S)-5-((1-benzylcyclopropyl)(methyl)amino)-1-(diethoxyphosphoryl)-1-hydroxy-5-oxopentan-2-yl)amino)-3-cyclohexyl-1-oxopropan-2-yl)carbamate (Compound C5). TLC system: 100% Ethyl acetate Rf: 0.3 LCMS (ESI): m/z 734.51 (M+H)⁺

### Example 29: Synthesis of Compounds C16 and C6

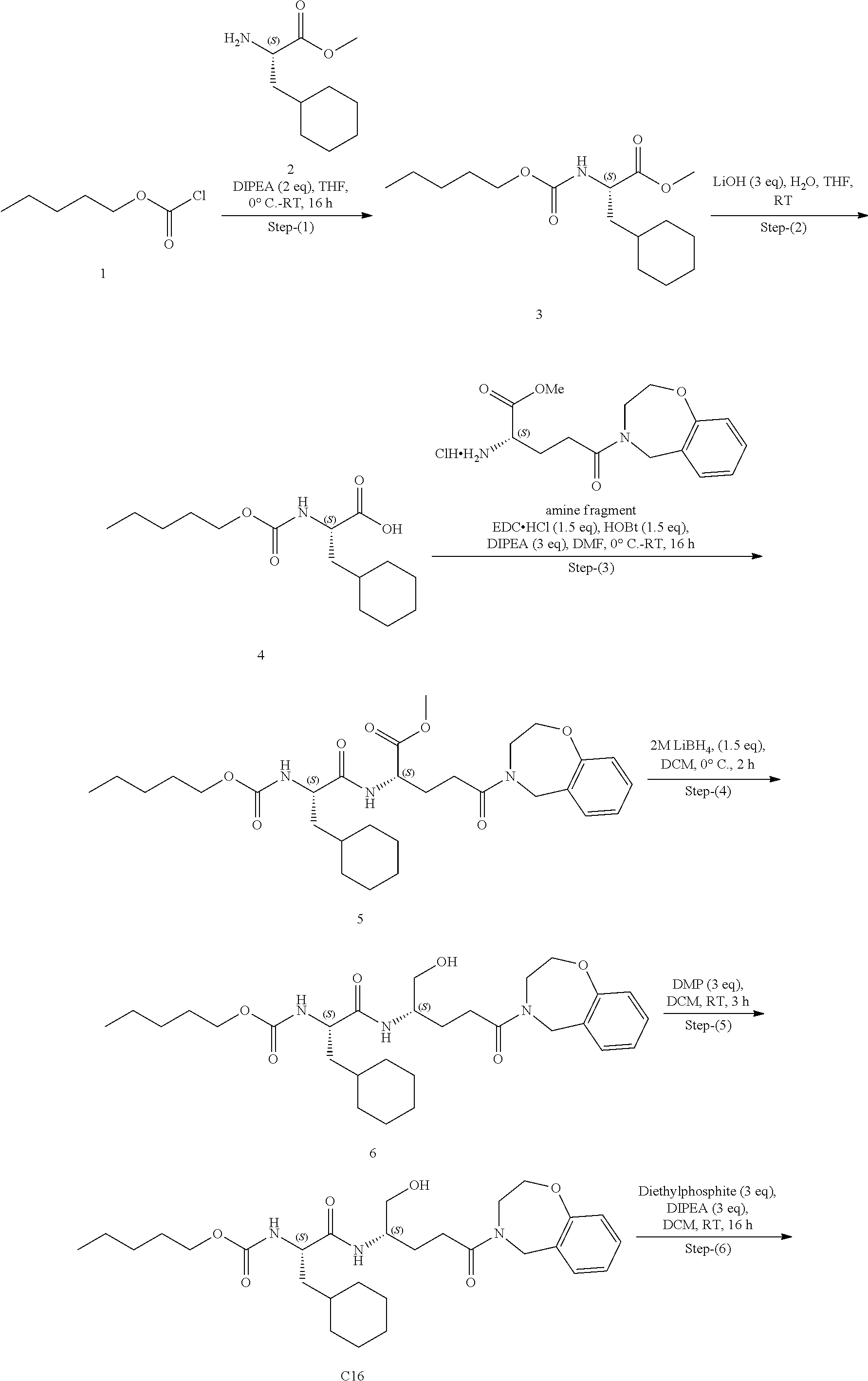

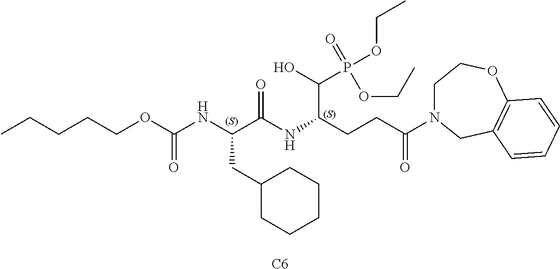

### Methyl (S)-3-cyclohexyl-2-(((pentyloxy)carbonyl)amino)propanoate (3)

To a stirred solution of (S)-2-amino-3-cyclohexylpropanoate hydrochloride (2) (3 g, 13.531 mmol), in THF (20 mL) and DIPEA (7 mL, 40.59 mmol) at 0° C. was added pentyl carbonochloridate (1) (2.34 mL, 16.2 mmol). The resulting mixture was stirred at RT for 2 h. The progress of the reaction was monitored by TLC and LCMS. After 2 h, reaction mixture was quenched with water (50 mL) and extracted with ethyl acetate (2×80 mL), the combined organic layer was dried over sodium sulfate, filtered and evaporated under reduced pressure. The crude residue was purified by silica gel column by eluting with 50% ethyl acetate in pet ether to afford methyl (S)-3-cyclohexyl-2-(((pentyloxy)carbonyl)amino)propanoate (3). TLC system: 30% Ethyl acetate in pet ether R_{f}: 0.55 LCMS (ESI): m/z 330.2 (M+NH)⁺

### (S)-3-Cyclohexyl-2-(((pentyloxy)carbonyl)amino)propanoic acid (4)

To a stirred solution of methyl (S)-3-cyclohexyl-2-(((pentyloxy)carbonyl)amino)propanoate (3) (2.5 g, 8.3 mmol) in THF (20 mL) and water (5 mL), was added lithium hydroxide (600 mg, 25 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH˜4, and extracted with dichloromethane (2×30 mL), dried over sodium sulfate, concentrated under reduced pressure to afford (S)-3-Cyclohexyl-2-(((pentyloxy)carbonyl)amino)propanoic acid (4). TLC system: 5% Methanol in DCM R_{f}: 0.2

### Methyl (S)-2-((S)-3-cyclohexyl-2-(((pentyloxy)carbonyl)amino)propanamido)-5-(2,3-dihydrobenzo[f][1,4]oxazepin-4(5H)-yl)-5-oxopentanoate (5)

To a stirred solution of (S)-3-cyclohexyl-2-(((pentyloxy)carbonyl)amino)propanoic acid (4) (1 g, 3.5 mmol) in DMF (20 mL) at 0° C. was added EDC.HCl (1 g, 5.2 mmol), HOBT (700 mg, 5.23 mmol), DIPEA (1.7 mL, 10.46 mmol) and methyl (S)-2-amino-5-(2,3-dihydrobenzo[f][1,4]oxazepin-4(5H)-yl)-5-oxopentanoate hydrochloride (Amine fragment) (1.38 g, 4.2 mmol) simultaneously and stirred at room temperature for 16 h. The progress of the reaction was monitored by TLC and LCMS. After 16 h, reaction mixture was quenched with ice water (30 mL), extracted with ethyl acetate (2×60 mL), the combined organic layer was dried over sodium sulfate and evaporated under reduced pressure. The crude residue was purified by silica gel column by eluting with 40% ethyl acetate in pet ether to afford methyl (S)-2-((S)-3-cyclohexyl-2-(((pentyloxy)carbonyl)amino)propanamido)-5-(2,3-dihydrobenzo[f][1,4]oxazepin-4(5H)-yl)-5-oxopentanoate (5). TLC system: 5% Methanol in DCM R_{f}: 0.6 LCMS (ESI): m/z 560.63 (M+H)⁺

### Pentyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6)

To a stirred solution of methyl (S)-2-((S)-3-cyclohexyl-2-(((pentyloxy)carbonyl)amino)propanamido)-5-(2,3-dihydrobenzo[f][1,4]oxazepin-4(5H)-yl)-5-oxopentanoate (5) (900 mg, 1.6 mmol) in DCM (10 mL) was added 2M LiBH₄ in THF (1.2 mL, 1.53 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford the crude pentyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6). TLC system: 5% MeOH in DCM R_{f} 0.3 LCMS (ESI): m/z 532.5 (M+H)⁺

### Pentyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C16)

To a stirred solution of pentyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6) (250 mg, 0.45 mmol) in DCM (5 mL) was added Dess-Martin periodinane (583 mg, 1.37 mmol) at 0° C. and stirred at RT for 3 h. The progress of the reaction was monitored by TLC and LCMS. Reaction mixture was diluted with DCM (50 mL) and washed with sat. NaHCO₃ solution (3×20 mL) followed by sat. Hypo solution (3×20 mL). Organic layer was dried over anhydrous Na₂SO₄, filtered and concentrated to get crude compound pentyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C16) which was used directly in the next step. TLC system: 5% Methanol in DCM R_{f}: 0.4 LCMS (ESI): m/z 530.56 (M+H)⁺

### Pentyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (Compound C6)

To a stirred solution of pentyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C16) (248 mg, 0.47 mmol) in DCM (5 mL) was added DIPEA (0.24 mL, 1.41 mmol) followed by diethylphosphate (0.19 mL, 1.41 mmol) and the reaction mixture stirred at RT for 16 h. The progress of the reaction was monitored by TLC and LCMS. After 16 h, the reaction mixture quenched with ammonium chloride (15 mL) and extracted with DCM (2×15 mL). Combined organic layer was dried over anhydrous Na₂SO₄, and evaporated to afford crude residue. It was purified by prep HPLC to Pentyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (Compound C6). TLC system: 5% MeOH in DCM R_{f}: 0.45 LCMS (ESI): m/z 668.68 (M+H)⁺

### Example 30: Synthesis of Compounds C26 and C7

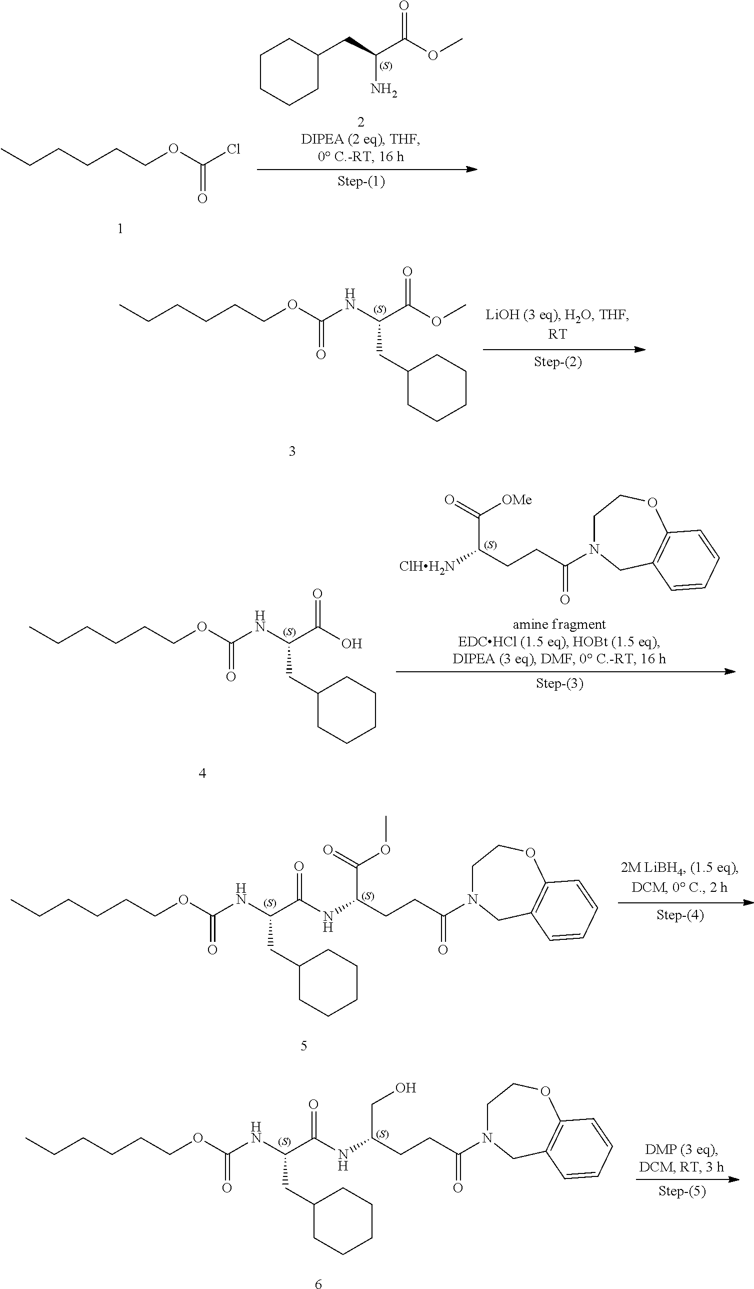

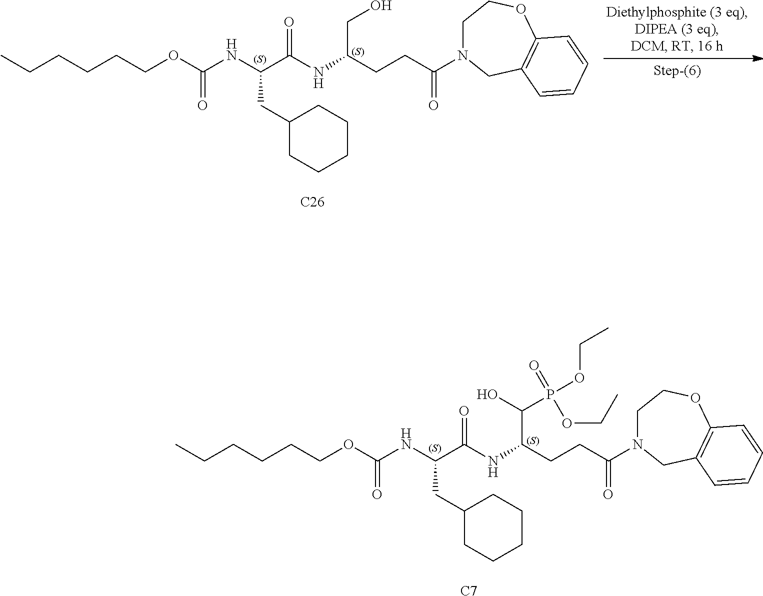

### Methyl (S)-3-cyclohexyl-2-(((hexyloxy)carbonyl)amino)propanoate (3)

To a stirred solution of (S)-2-amino-3-cyclohexylpropanoate hydrochloride (2) (4.8 g, 2.1 mmol), in THF (20 mL) and DIPEA (9.7 mL, 5.4 mmol) at 0° C. was added hexyl carbonochloridate (1) (3 g, 1.8 mmol). The resulting mixture was stirred at RT for 2 h. The progress of the reaction was monitored by TLC and LCMS. After 2 h, reaction mixture was quenched with water (50 mL) and extracted with ethyl acetate (2×80 mL), the combined organic layer was dried over sodium sulfate, filtered and evaporated under reduced pressure. The crude residue was purified by silica gel column by eluting with 50% ethyl acetate in pet ether to afford methyl (S)-3-cyclohexyl-2-(((hexyloxy)carbonyl)amino)propanoate (3). TLC system: 5% MeOH in DCM R_{f}: 0.55 LCMS (ESI): m/z 314.42 (M+H)⁺

### (S)-3-Cyclohexyl-2-(((hexyloxy)carbonyl)amino)propanoic acid (4)

To a stirred solution of methyl (S)-3-cyclohexyl-2-(((hexyloxy)carbonyl)amino)propanoate (3) (2 g, 6.36 mmol) in THF (20 mL) and water (5 mL), was added lithium hydroxide (450 mg, 19 mmol) at RT and stirred at room temperature for 3 h. The progress of the reaction was monitored by TLC and LCMS. After 3 h, the reaction mixture completely distilled under reduced pressure, crude compound acidified with aq. 1N HCl solution up to pH˜4, and extracted with dichloromethane (2×30 mL), dried over sodium sulfate, concentrated under reduced pressure to afford (S)-3-Cyclohexyl-2-(((hexyloxy)carbonyl)amino)propanoic acid (4). TLC system: 5% Methanol in DCM R_{f}: 0.2 LCMS (ESI): m/z 300.2 (M+H)⁺

### tert-Butyl 1-((S)-4-((S)-2-((((3-chlorobenzyl)oxy)carbonyl)amino)-3-cyclohexylpropanamido)-5-methoxy-5-oxopentanoyl)-1,2,3,5-tetrahydro-4H-benzo[e][1,4]diazepine-4-carboxylate (5)

At 0° C., to a stirred solution of (S)-3-cyclohexyl-2-(((hexyloxy)carbonyl)amino)propanoic acid (1.2 g, 4 mmol) in DMF (20 mL) was added EDC.HCl (1.14 g, 6 mmol), HOBT (834 mg, 6 mmol), DIPEA (2 mL, 12 mmol) and methyl (S)-2-amino-5-(2,3-dihydrobenzo[f][1,4]oxazepin-4(5H)-yl)-5-oxopentanoate hydrochloride (Amine fragment) (1.57 g, 4.8 mmol) and stirred at room temperature for 16 h. The progress of the reaction was monitored by TLC and LCMS. After 16 h, reaction mixture was quenched with ice water (500 mL), extracted with ethyl acetate (2×50 mL), the combined organic layer was dried over sodium sulfate and evaporated under reduced pressure. The crude residue was purified by silica gel column by eluting with 40% ethyl acetate in pet ether to afford tert-Butyl 1-((S)-4-((S)-2-((((3-chlorobenzyl)oxy)carbonyl)amino)-3-cyclohexylpropanamido)-5-methoxy-5-oxopentanoyl)-1,2,3,5-tetrahydro-4H-benzo[e][1,4]diazepine-4-carboxylate (5). TLC system: 5% Methanol in DCM R_{f}: 0.4 LCMS (ESI): m/z 574.53 (M+H)⁺

### Hexyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6)

To a stirred solution of methyl (S)-2-((S)-3-cyclohexyl-2-(((hexyloxy)carbonyl)amino)propanamido)-5-(2,3-dihydrobenzo[f][1,4]oxazepin-4(5H)-yl)-5-oxopentanoate (5) (960 mg, 1.67 mmol) in DCM (10 mL) was added 2M LiBH₄ in THF (1.25 mL, 1.5 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford the crude hexyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6). TLC system: 5% MeOH in DCM R_{f} 0.3 LCMS (ESI): m/z 546.51 (M+H)⁺

### Hexyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C26)

To a stirred solution of hexyl ((S)-3-cyclohexyl-1-(((S)-5-(2,3-dihydrobenzo[f][1,4]oxazepin-4(5H)-yl)-1-hydroxy-5-oxopentan-2-yl)amino)-1-oxopropan-2-yl)carbamate (6) (250 mg, 0.45 mmol) in DCM (5 mL) was added Dess-Martin periodinane (583 mg, 1.37 mmol) at 0° C. and stirred at RT for 3 h. The progress of the reaction was monitored by TLC and LCMS. Reaction mixture was diluted with DCM (50 mL) and washed with sat. NaHCO₃ solution (3×20 mL) followed by sat. Hypo solution (3×20 mL). Organic layer was dried over anhydrous Na₂SO₄, filtered and concentrated to get crude hexyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (Compound C26) which was used directly in the next step. TLC system: 5% Methanol in DCM R_{f}: 0.5 LCMS (ESI): m/z 544.55 (M+H)⁺

### Hexyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (Compound C7)

To a stirred solution of hexyl ((S)-3-cyclohexyl-1-(((S)-5-(2,3-dihydrobenzo[f][1,4]oxazepin-4(5H)-yl)-1,5-dioxopentan-2-yl)amino)-1-oxopropan-2-yl)carbamate (248 mg, 0.45 mmol) in DCM (5 mL) was added DIPEA (0.23 mL, 1.37 mmol) followed by diethylphosphate (0.18 mL, 1.37 mmol) and the reaction mixture stirred at RT for 16 h. The progress of the reaction was monitored by TLC and LCMS. After 16 h, the reaction mixture quenched with ammonium chloride (15 mL) and extracted with DCM (2×15 mL). Combined organic layer was dried over anhydrous Na₂SO₄, and evaporated to afford crude residue. It was purified prep HPLC to afford Hexyl ((2S)-3-cyclohexyl-1-(((2S)-1-(diethoxyphosphoryl)-5-(2,3-dihydrobenzo[f][1,4]oxazepin-4(5H)-yl)-1-hydroxy-5-oxopentan-2-yl)amino)-1-oxopropan-2-yl)carbamate (Compound C7). TLC system: 5% MeOH in DCM R_{f}: 0.5 LCMS (ESI): m/z 682.6 (M+H)⁺

### Example 31: Synthesis of Compounds C27 and C8

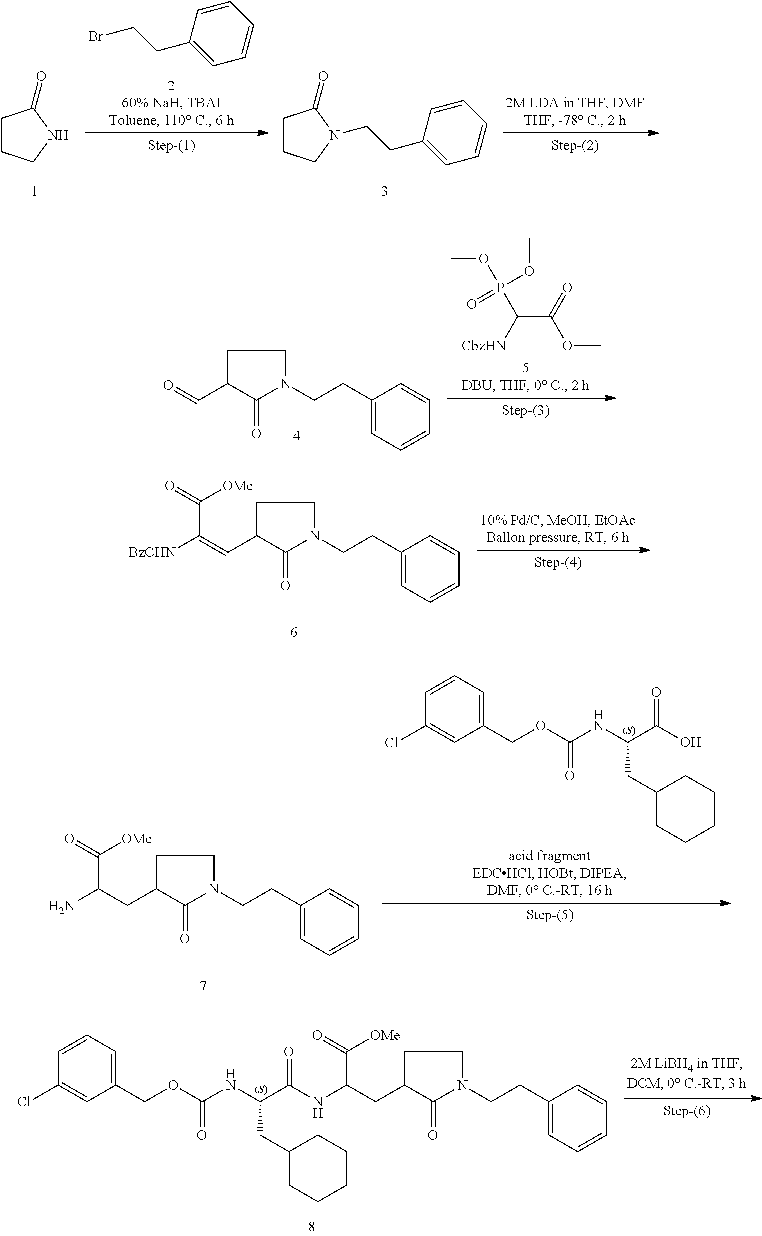

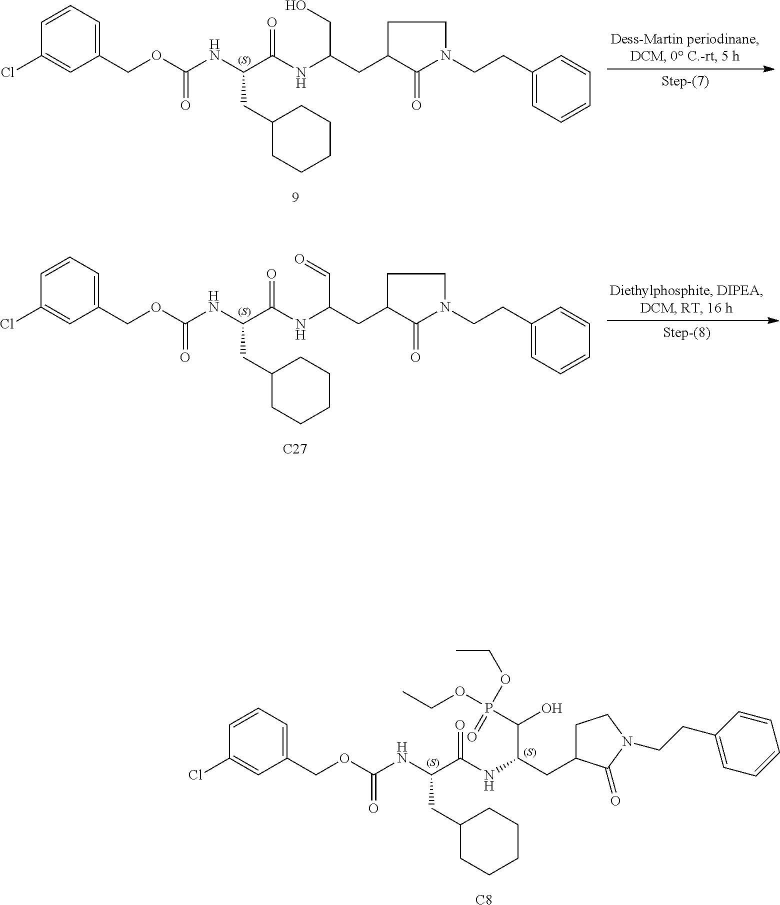

### 1-Phenethylpyrrolidin-2-one (3)

To a stirred solution of pyrrolidin-2-one (1) (10 g, 117.64 mmol) in toluene (150 mL) was added 60% NaH (7.0 g, 176.47 mmol), TBAI (8.68 g, 23.52 mmol) followed by (2-bromoethyl)benzene (2) (21.64 mL, 152.94 mmol) and refluxed for 6 h. Reaction mixture was quenched with ice water (150 mL), extracted with ethyl acetate (2×150 mL), dried over sodium sulfate and evaporated under reduced pressure. The crude residue was purified by silica gel column by eluting with 70% ethyl acetate in hexane to afford 1-phenethylpyrrolidin-2-one (3). TLC system: 80% Ethyl acetate in Hexane Rf: 0.2 LCMS (ESI): m/z 190.29 [M+H]⁺

### 2-Oxo-1-phenethylpyrrolidine-3-carbaldehyde (4)

To a stirred solution of 1-phenethylpyrrolidin-2-one (3) (4.0 g, 21.141 mmol) in THF (60 mL) was added 2M LDA in THF (16 mL, 31.71 mmol) with drop wise at −78° C. Reaction mixture was stirred at −78° C. for 1 h then DMF (2.3 mL, 31.712 mmol) in THF (10 mL) was added and stirred for 2 h at same temperature. The progress of the reaction was monitored by TLC. After consumption of starting material, the reaction mixture was quenched with sat. NH₄Cl solution, extracted with ethyl acetate (2×50 mL), dried over sodium sulfate and evaporated under reduced pressure to afford 2-oxo-1-phenethylpyrrolidine-3-carbaldehyde (4) which was used directly in the next step. TLC system: 80% Ethyl acetate in Hexane Rf: 0.4 LCMS (ESI): m/z 218.20 [M+H]⁺

### Methyl (E)-2-(((benzyloxy) carbonyl) amino)-3-(2-oxo-1-phenethylpyrrolidin-3-yl) acrylate (6)

To a stirred solution of 2-oxo-1-phenethylpyrrolidine-3-carbaldehyde (4) (4.5 g, crude) in THF (60 mL) was added methyl 2-(((benzyloxy)carbonyl)amino)-2-(dimethoxyphosphoryl) acetate (8.2 g, 24.86 mmol) followed by DBU 4.72 g, 31.07 mmol) at 0° C. and stirred for 2 h. Reaction mixture was diluted with ice water (50 mL), extracted with ethyl acetate (2×40 mL), dried over sodium sulfate and evaporated under reduced pressure. The crude residue was purified by silica gel column by eluting with 25% ethyl acetate in hexane to afford methyl (E)-2-(((benzyloxy)carbonyl)amino)-3-(2-oxo-1-phenethylpyrrolidin-3-yl)acrylate (6). TLC system: 50% Ethyl acetate in Hexane Rf: 0.5 LCMS (ESI): m/z 218.20 [M+H]⁺

### Methyl 2-amino-3-(2-oxo-1-phenethylpyrrolidin-3-yl) propanoate (7)

To a stirred solution of methyl (E)-2-(((benzyloxy) carbonyl) amino)-3-(2-oxo-1-phenethylpyrrolidin-3-yl) acrylate (6) (2.2 g, 5.213 mmol) in methanol (15 mL), ethyl acetate (15 mL) was added 10% Pd/C (500 mg) and stirred for 6 h under H₂ balloon pressure (15 Psi). The progress of the reaction was monitored by TLC and LCMS. After 6 h, the reaction mixture was filtered through celite bed and washed with ethyl acetate (30 mL), filtrate was concentrated under reduced pressure to afford methyl 2-amino-3-(2-oxo-1-phenethylpyrrolidin-3-yl)propanoate (7). TLC system: 50% Ethyl acetate in Hexane Rf: 0.4 LCMS (ESI): m/z 291.28 [M+H]⁺

### Methyl 2-((S)-2-((((3-chlorobenzyl) oxy) carbonyl) amino)-3-cyclohexylpropanamido)-3-(2-oxo-1-phenethylpyrrolidin-3-yl) propanoate (8)

To a stirred solution of (S)-2-((((3-chlorobenzyl)oxy)carbonyl)amino)-3-cyclohexyl propanoic acid (acid fragment) (1.0 g, 2.948 mmol) in DMF (15 mL) was added EDC.HCl (0.84 g, 4.42 mmol), HOBT (0.59 g, 4.42 mmol), DIPEA (1.14 mL, 8.84 mmol) and methyl 2-amino-3-(2-oxo-1-phenethylpyrrolidin-3-yl)propanoate (7) (0.5 g, 1.74 mmol) at 0° C. simultaneously and stirred at room temperature for 16 h. Reaction mixture was diluted with ice water (50 mL), extracted with ethyl acetate (2×40 mL), dried over sodium sulfate and evaporated under reduced pressure. The crude residue was purified by silica gel column by eluting with 2% methanol in dichloromethane to afford methyl 2-((S)-2-((((3-chlorobenzyl)oxy)carbonyl)amino)-3-cyclohexylpropanamido)-3-(2-oxo-1-phenethylpyrrolidin-3-yl)propanoate (8). TLC system: 5% Methanol in dichloromethane Rf: 0.4 LCMS (ESI): m/z 612.47 (M+H)⁺

### 3-Chlorobenzyl ((2S)-3-cyclohexyl-1-((1-hydroxy-3-(2-oxo-1-phenethylpyrrolidin-3-yl) propan-2-yl) amino)-1-oxopropan-2-yl) carbamate (9)

To a stirred solution of methyl 2-((S)-2-((((3-chlorobenzyl)oxy)carbonyl)amino)-3-cyclohexylpropanamido)-3-(2-oxo-1-phenethylpyrrolidin-3-yl)propanoate (8) (400 mg, 0.65 mmol) in DCM (4 mL) was added 2M LiBH₄ in THF (0.65 mL, 1.30 mmol) at 0° C. and the reaction mixture stirred at same temperature for 2 h. The progress of the reaction was monitored by TLC and LCMS. Then reaction mixture was quenched with sat. ammonium chloride solution (20 mL) and extracted with ethyl acetate (2×20 mL). Organic layer was washed with brine solution (20 mL), dried over Na₂SO₄ and concentrated to afford crude. The crude residue was purified by silica gel column by eluting with 2% methanol in dichloromethane to afford 3-chlorobenzyl ((2S)-3-cyclohexyl-1-((1-hydroxy-3-(2-oxo-1-phenethylpyrrolidin-3-yl) propan-2-yl) amino)-1-oxopropan-2-yl) carbamate (9). TLC system: 5% Methanol in dichloromethane Rf: 0.2 LCMS (ESI): m/z 584.45 (M+H)⁺

### 3-Chlorobenzyl ((2S)-3-cyclohexyl-1-oxo-1-((1-oxo-3-(2-oxo-1-phenethylpyrrolidin-3-yl) propan-2-yl) amino) propan-2-yl) carbamate (Compound C27)

To a stirred solution of 3-chlorobenzyl ((2S)-3-cyclohexyl-1-((1-hydroxy-3-(2-oxo-1-phenethylpyrrolidin-3-yl) propan-2-yl) amino)-1-oxopropan-2-yl) carbamate (9) (200 mg, 0.34 mmol) in dichloromethane (5 mL) was added Dess-Martin periodinane (436 mg, 1.02 mmol) at 0° C. and stirred at RT for 3 h. The progress of the reaction was monitored by TLC and LCMS. Reaction mixture was diluted with dichloromethane (15 mL) and washed with sat. Hypo solution (3×10 mL), sat. NaHCO₃ solution (3×20 mL). Organic layer was dried over anhydrous Na₂SO₄, filtered and concentrated to get crude 3-chlorobenzyl ((2S)-3-cyclohexyl-1-oxo-1-((1-oxo-3-(2-oxo-1-phenethylpyrrolidin-3-yl) propan-2-yl) amino) propan-2-yl) carbamate (Compound C27) which was used directly in the next step. TLC system: 10% Methanol in DCM Rf: 0.4 LCMS (ESI): m/z 582.52 (M+H)⁺

### 3-Chlorobenzyl ((2S)-3-cyclohexyl-1-((1-(diethoxyphosphaneyl)-1-hydroxy-3-(2-oxo-1-phenethylpyrrolidin-3-yl) propan-2-yl) amino)-1-oxopropan-2-yl) carbamate (Compound C8)

To a stirred solution of crude 3-chlorobenzyl ((2S)-3-cyclohexyl-1-oxo-1-((1-oxo-3-(2-oxo-1-phenethylpyrrolidin-3-yl) propan-2-yl) amino) propan-2-yl) carbamate (Compound C27) (220 mg, 0.378 mmol) in DCM (5 mL) was added DIPEA (0.2 mL, 1.13 mmol) and diethylphosphate (0.2 mL, 1.13 mmol) at 0° C. and stirred at RT for 16 h. The progress of the reaction was monitored by TLC and LCMS. Reaction mixture was quenched with ice water (15 mL) extracted with DCM (3×15 mL). Organic layer was dried over anhydrous Na₂SO₄, filtered and purified by prep HPLC afforded 3-chlorobenzyl ((2S)-3-cyclohexyl-1-(((2S)-1-(diethoxyphosphoryl)-1-hydroxy-3-(2-oxo-1-phenethylpyrrolidin-3-yl)propan-2-yl)amino)-1-oxopropan-2-yl) carbamate (Compound C8). TLC system: 10% Methanol in DCM Rf: 0.5 LCMS (ESI): m/z 720.58 (M+H)⁺

### Example 32: Synthesis of Compound C35 and C31

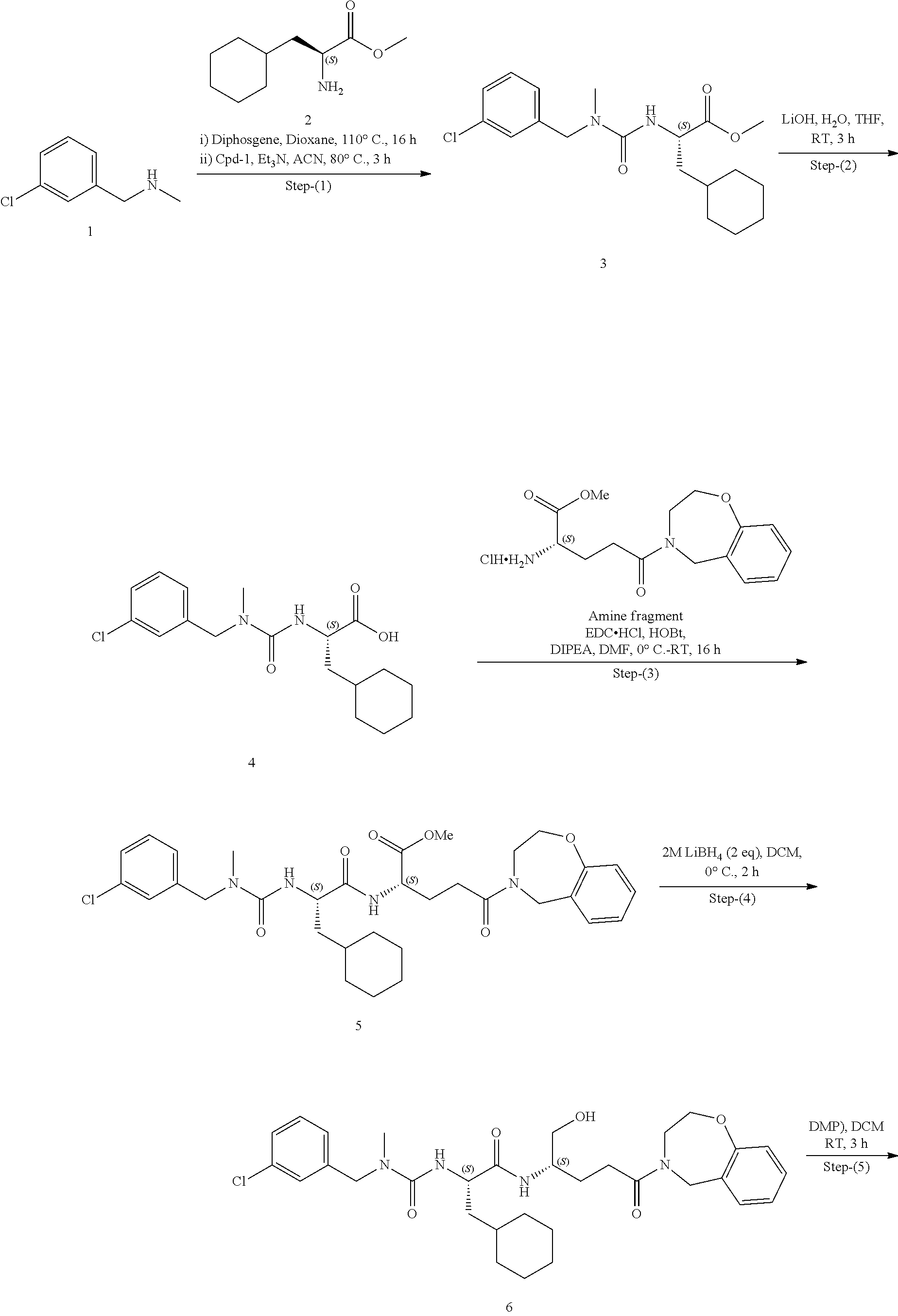

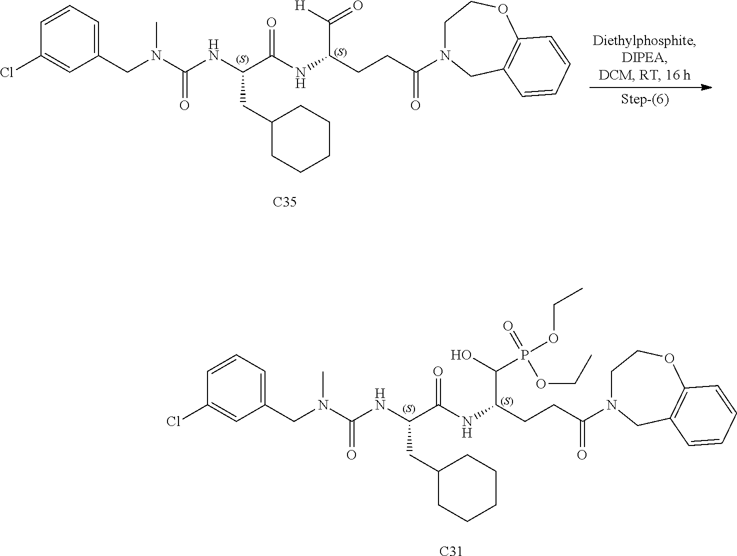

### Methyl (S)-2-(3-(3-chlorobenzyl)-3-methylureido)-3-cyclohexylpropanoate (3)

To a stirred solution of methyl (S)-2-amino-3-cyclohexylpropanoate hydrochloride (2) (500 mg, 2.26 mmol) in 1,4 dioxane (10 mL) added diphosgene (0.4 mL, 3.39 mmol) at RT and heated to reflux for 16 h. The progress of the reaction was monitored by TLC. Reaction mixture was evaporated under reduced pressure to afford crude residue, this residue was used directly in the subsequent reaction.

In another RB flask, 1-(3-chlorophenyl)-N-methylmethanamine (1) (350 mg, 2.26 mmol) was taken in ACN (10 mL), and treated with triethylamine (0.95 mL, 6.78 mmol). The resulting reaction mixture was stirred for 5 min, then added above prepared reaction mass drop-wise and the reaction mixture stirred at RT and heated to 80° C. 3 h, the reaction mixture was quenched with ice water (20 mL) and extracted with ethyl acetate (2×20 mL), combined organic layers were washed with brine solution (20 mL), dried over sodium sulfate and evaporated under reduced pressure. The crude residue was purified by normal phase chromatography to afford methyl (S)-2-(3-(3-chlorobenzyl)-3-methylureido)-3-cyclohexylpropanoate (3). TLC system: 50% Ethyl acetate in hexane Rf: 0.3 LCMS (ESI): m/z 367.32 [M+H]⁺

### (S)-2-(3-(3-Chlorobenzyl)-3-methylureido)-3-cyclohexylpropanoic acid (4)

To a stirred solution of (S)-2-(3-(3-chlorobenzyl)-3-methylureido)-3-cyclohexylpropanoate (3) (600 mg, 1.63 mmol) in THF (4 mL), water (2 mL) was added lithium hydroxide (117 mg, 4.89 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×15 mL), dried over sodium sulfate, concentrated under reduced pressure to afford (S)-2-(3-(3-chlorobenzyl)-3-methylureido)-3-cyclohexylpropanoic acid (4). TLC system: 100% EtOAc Rf: 0.1 LCMS (ESI): m/z 353.47 [M+H]⁺

### Methyl (S)-2-((S)-2-(3-(3-chlorobenzyl)-3-methylureido)-3-cyclohexylpropanamido)-5-(2,3-dihydrobenzo[f][1,4] oxazepin-4(5H)-yl)-5-oxopentanoate (5)

To a stirred solution of (S)-2-(3-(3-chlorobenzyl)-3-methylureido)-3-cyclohexylpropanoic acid (500 mg, 1.42 mmol) DMF (10 mL) added EDC.HCl (406 mg, 2.13 mmol), HOBt (287 mg, 2.13 mmol), DIPEA (0.7 mL, 4.26 mmol) and methyl (S)-2-amino-5-(2,3-dihydrobenzo[f][1,4]oxazepin-4(5H)-yl)-5-oxopentanoate hydrochloride (amine fragment) (559 mg, 1.70 mmol) at 0° C. simultaneously and stirred at room temperature for 16 h. Reaction mixture was diluted with ice water (20 mL), extracted with ethyl acetate (2×20 mL), dried over sodium sulfate and evaporated under reduced pressure. The crude residue was purified by combi-flash NP, compound eluted at 2% methanol in dichloromethane to afford methyl (S)-2-((S)-2-(3-(3-chlorobenzyl)-3-methylureido)-3-cyclohexylpropanamido)-5-(2,3-dihydrobenzo[f][1,4]oxazepin-4(5H)-yl)-5-oxopentanoate (5). TLC system: 5% Methanol in dichloromethane Rf: 0.4 LCMS (ESI): m/z 627.89 [M+H]⁺

### (S)-2-(3-(3-Chlorobenzyl)-3-methylureido)-3-cyclohexyl-N—((S)-5-(2,3-dihydrobenzo[f][1, 4]oxazepin-4(5H)-yl)-1-hydroxy-5-oxopentan-2-yl)propanamide (6)

To a stirred solution of methyl (S)-2-((S)-2-(3-(3-chlorobenzyl)-3-methylureido)-3-cyclohexylpropanamido)-5-(2,3-dihydrobenzo[f][1,4]oxazepin-4(5H)-yl)-5-oxopentanoate (5) (480 mg, 0.95 mmol) in DCM (10 mL) was added 2M LiBH₄ in THF (0.95 mL, 1.91 mmol) at 0° C. and the reaction mixture stirred for 2 h at 0° C. The progress of the reaction was monitored by TLC and LCMS. Reaction mixture was quenched with sat. Ammonium chloride solution (20 mL) and extracted with DCM (2×20 mL). Organic layer was washed with brine solution (20 mL), dried over Na₂SO₄ and concentrated to afford (S)-2-(3-(3-chlorobenzyl)-3-methylureido)-3-cyclohexyl-N—((S)-5-(2,3-dihydrobenzo[f][1,4] oxazepin-4(5H)-yl)-1-hydroxy-5-oxopentan-2-yl) propanamide (6). TLC system: 5% Methanol in dichloromethane Rf: 0.2 LCMS (ESI): m/z 599.53 [M+H]⁺

### (S)-2-(3-(3-Chlorobenzyl)-3-methylureido)-3-cyclohexyl-N—((S)-5-(2,3-dihydrobenzo[f][1,4]oxazepin-4(5H)-yl)-1,5-dioxopentan-2-yl)propanamide (Compound C35)

To a stirred solution of (S)-2-(3-(3-chlorobenzyl)-3-methylureido)-3-cyclohexyl-N—((S)-5-(2,3-dihydrobenzo[f][1,4] oxazepin-4(5H)-yl)-1-hydroxy-5-oxopentan-2-yl) propanamide (6) (200 mg, 0.33 mmol) was dissolved in dichloromethane (5 mL) was added Dess-Martin periodinane (425 mg, 1.00 mmol) at 0° C. and stirred at RT for 3 h. Reaction mixture was diluted with DCM (15 mL) followed by sat. Hypo solution (3×15 mL), followed by sat. NaHCO₃ solution (3×15 mL). Organic layer was dried over anhydrous Na₂SO₄, filtered and concentrated to get crude compound. The crude compound was purified by prep HPLC to afford (S)-2-(3-(3-chlorobenzyl)-3-methylureido)-3-cyclohexyl-N—((S)-5-(2,3-dihydrobenzo[f][1,4]oxazepin-4(5H)-yl)-1,5-dioxopentan-2-yl)propanamide (Compound C35). TLC system: 10% Methanol in dichloromethane Rf: 0.5 LCMS (ESI): m/z 597.44 (M+H)⁺

### Diethyl ((2S)-2-((S)-2-(3-(3-chlorobenzyl)-3-methylureido)-3-cyclohexylpropanamido)-5-(2,3-dihydrobenzo[f][1,4]oxazepin-4(5H)-yl)-1-hydroxy-5-oxopentyl)phosphonate (Compound C31)

To a stirred solution of (S)-2-(3-(3-chlorobenzyl)-3-methylureido)-3-cyclohexyl-N—((S)-5-(2,3-dihydrobenzo[f][1,4]oxazepin-4(5H)-yl)-1,5-dioxopentan-2-yl)propanamide (Compound C35) (110 mg crude, 0.18 mmol) in DCM (4 mL) added DIPEA (0.1 mL, 0.55 mmol) followed by added diethyl phosphite (76 mg, 0.55 mmol) and the reaction mixture stirred at RT for 16 h. The progress of the reaction was monitored by TLC and LCMS. After 16 h, the reaction mixture quenched with ammonium chloride (10 mL) and extracted with DCM (2×15 mL). Combined organic layer was dried over anhydrous Na₂SO₄, and evaporated to afford crude residue. It was purified prep HPLC to afford diethyl ((2S)-2-((S)-2-(3-(3-chlorobenzyl)-3-methylureido)-3-cyclohexylpropanamido)-5-(2,3-dihydrobenzo[f][1,4] oxazepin-4(5H)-yl)-1-hydroxy-5-oxopentyl) phosphonate (Compound C31). TLC system: 10% Methanol in dichloromethane Rf: 0.4 LCMS (ESI): m/z 735.44 (M+H)⁺

### Example 33: Synthesis of Compound C32

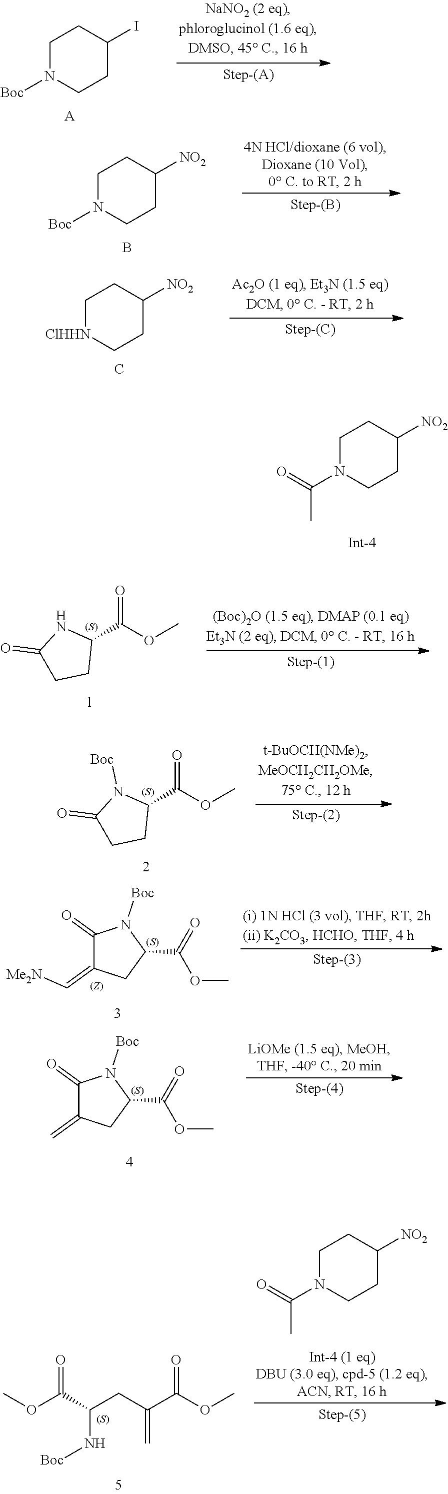

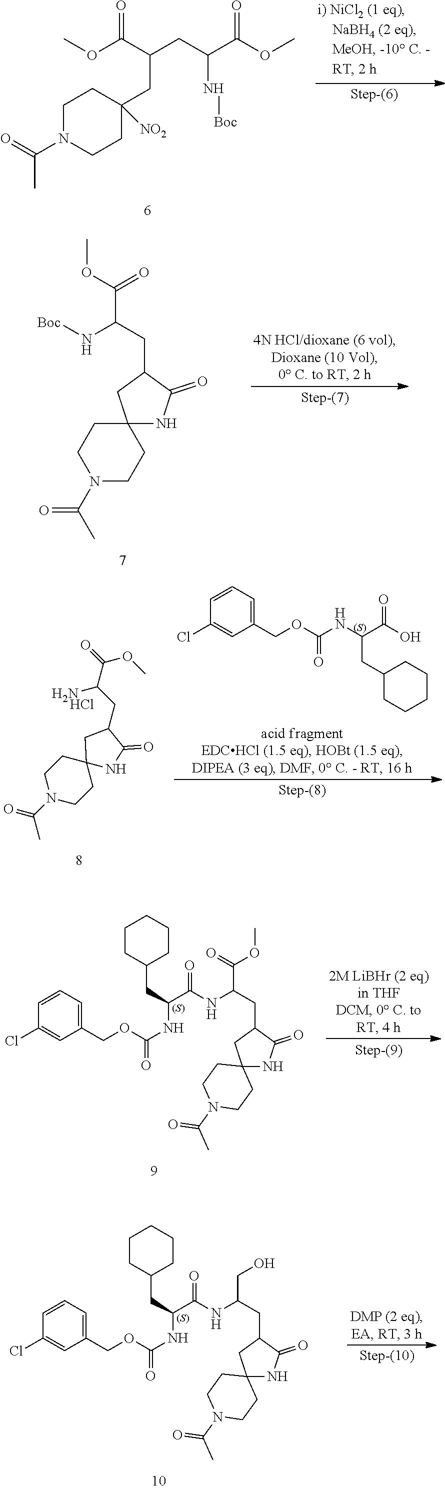

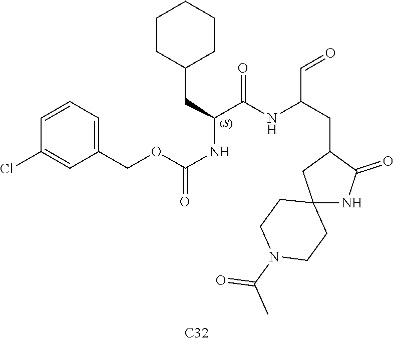

### Tert-butyl 4-nitropiperidine-1-carboxylate (B)

To a stirred solution of tert-butyl 4-iodopiperidine-1-carboxylate (A) (20 g, 64.308 mmol) in DMSO (100 mL) was added phloroglucinol (12.9 g, 102.89 mmol), followed by sodium nitrite (8.8 g, 128.6 mmol) at room temperature and stirred at 45° C. for 16 h. The progress of the reaction was monitored by TLC. The reaction mixture was quenched with water (250 mL) and extracted with diethyl ether (3×100 mL), combined organic layers were washed with water (2×50 mL), brine solution (50 mL), dried over sodium sulfate and evaporated under reduced pressure. The crude residue was purified by normal phase chromatography to afford tert-butyl 4-nitropiperidine-1-carboxylate (B). TLC system: 50% EtOAc/Pet ether R_{f}: 0.45

### 4-Nitropiperidine hydrochloride (C)

To a stirred solution of tert-butyl 4-nitropiperidine-1-carboxylate (B) (3 g, 13.043 mmol) in 1,4-dioxane (5 mL) was added 4N HC (5 mL) at 0° C. and stirred at room temperature for 4 h. The progress of the reaction was monitored by TLC and LCMS. After 4 h, the reaction mixture completely distilled under reduced pressure, crude compound was triturated with diethyl ether (2×10 mL) to afford 4-nitropiperidine hydrochloride (C). TLC system: 5% MeOH/DCM R_{f}: 0.1 LCMS (ESI): m/z 131.10 [M+H]⁺

### 1-(4-Nitropiperidin-1-yl)ethan-1-one (Int-4)

To a stirred solution of 4-nitropiperidine hydrochloride (C) (2.2 g, 13.25 mmol) was dissolved in DCM (20 mL) was added acetic anhydride (1.25 mL, 13.25 mmol) and triethyl amine (2.7 mL, 19.87 mmol) at 0° C. simultaneously and stirred at RT for 2 h. The progress of the reaction was monitored by TLC and LCMS. Reaction mixture was quenched with ice water (20 mL) extracted with DCM (2×20 mL), organic layers were washed with water (2×10 mL), brine solution (10 mL), dried over sodium sulfate and evaporated under reduced pressure. The crude residue was purified by normal phase chromatography to afford the title compound. TLC system: 30% EtOAc in pet ether Rf: 0.3 LCMS (ESI): m/z 173.33 [M+H]⁺

### 1-(tert-butyl) 2-methyl (S)-5-oxopyrrolidine-1,2-dicarboxylate (2)

To a stirred solution of ethyl (S)-5-oxopyrrolidine-2-carboxylate (1) (20.0 g, 127.38 mmol) in DCM (200 mL) was added Triethylamine (22.02 mL, 152.86 mmol), Boc anhydride (30.54 mL, 140.12 mmol) and DMAP (1.5 g, 12.73 mmol) at 0° C. and reaction mixture was stirred at room temperature for 16 h. The progress of the reaction was monitored by TLC, the reaction mixture was quenched with ice water (500 mL) and extracted with Dichloromethane (3×400 mL) dried over sodium sulfate and concentrated under reduced pressure. The crude residue was purified by grace NP, compound eluted with 30% ethyl acetate and pet ether to afford 1-(tert-butyl) 2-ethyl (S)-5-oxopyrrolidine-1,2-dicarboxylate (2). TLC system: 30% Ethyl acetate in pet ether Rf: 0.3 LCMS (ESI): m/z 258.23 (M+H)⁺

### 1-(tert-butyl) 2-methyl (S,Z)-4-((dimethylamino)methylene)-5-oxopyrrolidine-1,2-dicarboxylate (3)

To a stirred solution of 1-(tert-butyl) 2-methyl (S)-5-oxopyrrolidine-1,2-dicarboxylate (2) (10 g, 41.15 mmol) in dimethoxymethane (100 mL) was added Bredereck reagent (13 mL, 61.72 mmol) at 0° C. and stirred at 80° C. for 3 h. The progress of the reaction was monitored by TLC and LCMS. The reaction mixture completely distilled under reduced pressure, crude compound was triturated with diethyl ether (2×20 mL) to afford 1-(tert-butyl) 2-methyl (S,Z)-4-((dimethylamino)methylene)-5-oxopyrrolidine-1,2-dicarboxylate (3). TLC system: 60% EtOAc in pet ether Rf: 0.2

### 1-(tert-butyl) 2-methyl (S)-4-methylene-5-oxopyrrolidine-1,2-dicarboxylate (4)

To a stirred solution of 1-(tert-butyl) 2-methyl (S,Z)-4-((dimethylamino)methylene)-5-oxopyrrolidine-1,2-dicarboxylate (3) (500 mg, 1.666 mmol) in THF (4 mL) was added 1N HCl (1.75 mL) at room temperature and stirred for 3 h. The progress of the reaction was monitored by TLC. Separated the layers and organic layer was used directly in the subsequent reaction.

In another RB flask, to the above organic layer added 33% formaldehyde (3 V) and potassium carbonate (344 mg, 2.49 mmol) at 0° C. and stirred at RT for 4 h. The progress of the reaction was monitored by TLC and LCMS. Separated the layers, aqueous layer extracted with ethyl acetate (2×100 mL), combined organic layers were washed with sat. Sodium bicarbonate (100 mL), brine solution (50 mL), dried over sodium sulfate and evaporated under reduced pressure. The crude residue was purified by normal phase chromatography to afford 1-(tert-butyl) 2-methyl (S)-4-methylene-5-oxopyrrolidine-1,2-dicarboxylate. TLC system: 30% EtOAc/Pet ether R_{f}: 0.2

### Dimethyl (S)-2-((tert-butoxycarbonyl)amino)-4-methylenepentanedioate (5)

To a stirred solution of 1-(tert-butyl) 2-methyl (S)-4-methylene-5-oxopyrrolidine-1,2-dicarboxylate (4) (400 mg, 1.56 mmol) in Dry THF (10 mL) was added lithium methoxide (1M in Methanol) (1.88 mL, 1.88 mmol) at −40° C. and stirred at same for 20 min. The progress of the reaction was monitored by TLC Reaction mixture was quenched with sat ammonium chloride (5 mL), extracted with ethyl acetate (3×20 mL), combined organic layers were washed with brine solution (10 mL), dried over sodium sulfate and evaporated under reduced pressure. The crude residue was purified by normal phase chromatography to afford dimethyl (S)-2-((tert-butoxycarbonyl)amino)-4-methylenepentanedioate (5). TLC system: 30% EtOAc in pet ether Rf: 0.3 LCMS (ESI): m/z 310.3 (M+Na+H)⁺

### Dimethyl 2-((1-acetyl-4-nitropiperidin-4-yl)methyl)-4-((tert-butoxycarbonyl)amino)pentanedioate (6)

To a stirred solution of dimethyl (S)-2-((tert-butoxycarbonyl)amino)-4-methylenepentanedioate (5) (500 mg, 1.74 mmol) in ACN (5 mL) was added 1-(4-nitropiperidin-1-yl)ethan-1-one (Int-4) (300 mg, 1.74 mmol) and DBU (0.55 mL, 3.48 mmol) at 0° C. and stirred at RT for 16 h. The progress of the reaction was monitored by TLC and LCMS. Reaction mixture was evaporated under reduced pressure. The crude residue was purified by normal phase chromatography to afford dimethyl 2-((1-acetyl-4-nitropiperidin-4-yl)methyl)-4-((tert-butoxycarbonyl)amino)pentanedioate (6). TLC system: 5% MeOH/DCM R_{f}: 0.35 LCMS (ESI): m/z 482.4 (M+H)⁺

### Methyl 3-(8-acetyl-2-oxo-1,8-diazaspiro[4.5]decan-3-yl)-2-((tert-butoxycarbonyl)amino)propanoate (7)

To a stirred solution of dimethyl 2-((1-acetyl-4-nitropiperidin-4-yl)methyl)-4-((tert-butoxycarbonyl)amino)pentanedioate (6) (100 mg, 0.217 mmol) in methanol (4 mL) was added nickel chloride (31 mg, 0.23 mmol), followed by sodiumborohydride (42 mg, 1.08 mmol) at −10° C. and stirred at RT for 2 h. The progress of the reaction was monitored by TLC and LCMS. The reaction mixture was quenched with water (5 mL) and extracted with ethyl acetate (3×10 mL), combined organic layers were washed with water (2×10 ml), brine solution (10 mL), dried over sodium sulfate and evaporated under reduced pressure. The crude residue was purified by normal phase chromatography to afford methyl 3-(8-acetyl-2-oxo-1,8-diazaspiro[4.5]decan-3-yl)-2-((tert-butoxycarbonyl)amino)propanoate (7). TLC system: 10% MeOH/DCM R_{f}: 0.2 LCMS (ESI): m/z 420.35 (M+Na+H)⁺

### Methyl 3-(8-acetyl-2-oxo-1,8-diazaspiro[4.5]decan-3-yl)-2-aminopropanoate hydrochloride (8)

To a stirred solution of methyl 3-(8-acetyl-2-oxo-1,8-diazaspiro[4.5]decan-3-yl)-2-((tert-butoxycarbonyl)amino)propanoate (7) (80 mg, 0.2 mmol) in dioxane (2 mL) was added 4M HCl in dioxane (2 mL) at 0° C. and stirred at RT for 2 h. The progress of the reaction was monitored by TLC. Reaction mixture was evaporated under reduced pressure. The crude residue was trituration with n-pentane to afford methyl 3-(8-acetyl-2-oxo-1,8-diazaspiro[4.5]decan-3-yl)-2-aminopropanoate hydrochloride (8). TLC system: 15% MeOH/DCM R_{f}: 0.1

### Methyl 3-(8-acetyl-2-oxo-1,8-diazaspiro[4.5]decan-3-yl)-2-((S)-2-((((3-chlorobenzyl)oxy)carbonyl)amino)-3-cyclohexylpropanamido)propanoate (9)

To a stirred solution of (S)-2-((((3-chlorobenzyl)oxy)carbonyl)amino)-3-cyclohexylpropanoic acid (Acid fragment) (54 mg, 0.16 mmol) in DMF (5 mL) at 0° C. was added EDC.HCl (42 mg, 0.22 mmol), HOBT (30 mg, 0.22 mmol), DIPEA (0.1 mL, 0.44 mmol) and methyl 3-(8-acetyl-2-oxo-1,8-diazaspiro[4.5]decan-3-yl)-2-aminopropanoate hydrochloride (8) (50 mg, 0.14 mmol) simultaneously and stirred at room temperature for 16 h. The progress of the reaction was monitored by TLC and LCMS. After 16 h, reaction mixture was quenched with ice water (20 mL), extracted with ethyl acetate (2×30 mL), the combined organic layer was dried over sodium sulfate and evaporated under reduced pressure. The crude residue was purified by silica gel column by eluting with 50% ethyl acetate in pet ether to afford methyl 3-(8-acetyl-2-oxo-1,8-diazaspiro[4.5]decan-3-yl)-2-((S)-2-((((3-chlorobenzyl)oxy)carbonyl)amino)-3-cyclohexylpropanamido)propanoate (9). TLC system: 5% Methanol in DCM R_{f}: 0.6 LCMS (ESI): m/z 619.57 (M+H)⁺

### 3-Chlorobenzyl ((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)carbamate (10)

To a stirred solution of methyl 3-(8-acetyl-2-oxo-1,8-diazaspiro[4.5]decan-3-yl)-2-((S)-2-((((3-chlorobenzyl)oxy)carbonyl)amino)-3-cyclohexylpropanamido)propanoate (9) (90 mg, 0.14 mmol) in DCM (10 mL) was added 2M LiBH₄ in THF (0.2 mL, 0.24 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford the 3-chlorobenzyl ((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)carbamate (10). TLC system: 5% MeOH in DCM R_{f} 0.3 LCMS (ESI): m/z 591.43 (M+H)⁺

### 3-Chlorobenzyl ((2S)-1-((1-(8-acetyl-2-oxo-1,8-diazaspiro[4.5]decan-3-yl)-3-oxopropan-2-yl)amino)-3-cyclohexyl-1-oxopropan-2-yl)carbamate (Compound C32)

To a stirred solution of 3-chlorobenzyl ((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)carbamate (10) (80 mg, 0.13 mmol) in ethyl acetate (5 mL) was added Dess-Martin periodinane (230 mg, 0.54 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 3-chlorobenzyl ((2S)-1-((1-(8-acetyl-2-oxo-1,8-diazaspiro[4.5]decan-3-yl)-3-oxopropan-2-yl)amino)-3-cyclohexyl-1-oxopropan-2-yl)carbamate (Compound C32). TLC system: 5% Methanol in DCM R_{f}: 0.4 LCMS (ESI): m/z 589.46 (M+H)⁺

### Example 34: Synthesis of Compounds C51 and C34

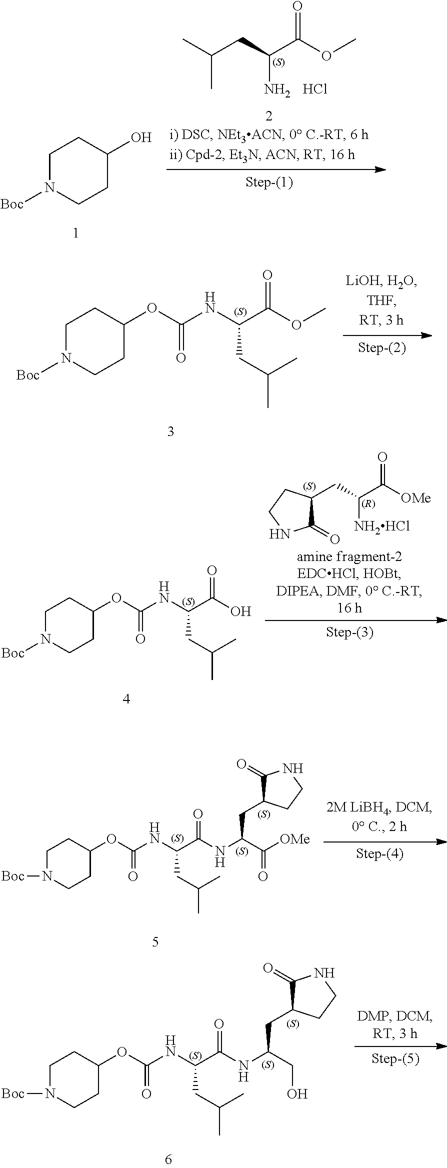

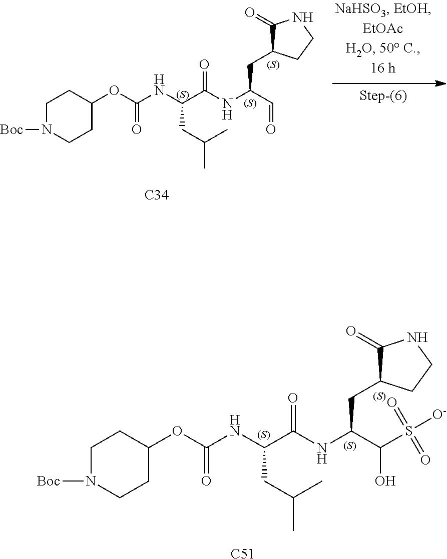

### Tert-butyl (S)-4-(((1-methoxy-4-methyl-1-oxopentan-2-yl) carbamoyl) oxy) piperidine-1-carboxylate (3)

To a stirred solution of tert-butyl 4-hydroxypiperidine-1-carboxylate (1) (10 g, 49.67 mmol) in ACN (80 mL) was added N,N′ disuccinimidyl carbonate (19.7 g, 74.51 mmol), followed by triethylamine (20.9 mL, 149.1 mmol) at 0° C. and stirred room temperature for 16 h. The progress of the reaction was monitored by TLC. The reaction mass was used directly in the subsequent reaction.

In another RB flask, methyl L-leucinate hydrochloride (2) (13.5 g, 74.58 mmol) was taken in ACN (50 mL), and treated with triethylamine (20.9 mL, 149.1 mmol). The resulting reaction mixture was stirred for 5 min, then added above prepared reaction mass drop-wise and the reaction mixture stirred at room temperature for 16 h. After 16 h, the reaction mixture was quenched with ice water (150 mL) and extracted with ethyl acetate (2×150 mL), combined organic layers were washed with brine solution (100 mL), dried over sodium sulfate and evaporated under reduced pressure.

The crude residue was purified by normal phase chromatography to afford Tert-butyl (S)-4-(((1-methoxy-4-methyl-1-oxopentan-2-yl) carbamoyl) oxy) piperidine-1-carboxylate (3). TLC system: 30% Ethyl acetate in Pet ether Rf: 0.5 LCMS (ESI): m/z 395.29 [M+Na]⁻

### (((1-(Teri-butoxycarbonyl) piperidin-4-yl) oxy) carbonyl)-L-leucined (4)

To a stirred solution of tert-butyl (S)-4-(((1-methoxy-4-methyl-1-oxopentan-2-yl)carbamoyl)oxy)piperidine-1-carboxylate (3) (3.5 g, 9.40 mmol) in THF (20 mL), water (5 mL) was added lithium hydroxide (1.18 g, 28.2 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford (((1-(tert-butoxycarbonyl)piperidin-4-yl)oxy)carbonyl)-L-leucine (4). TLC system: 10% Methanol in dichloromethane Rf: 0.1 LCMS (ESI): m/z=381.53 [M+Na]⁺

### Tert-butyl 4-((((S)-1-(((S)-1-methoxy-1-oxo-3-((S)-2-oxopyrrolidin-3-yl) propan-2-yl) amino)-4-methyl-1-oxopentan-2-yl) carbamoyl) oxy) piperidine-1-carboxylate (5)

To a stirred solution of (((1-(tert-butoxycarbonyl)piperidin-4-yl)oxy)carbonyl)-L-leucine (4) (2.0 g, 5.58 mmol) in DMF (20 mL) added EDC.HCl (1.6 g, 8.37 mmol), HOBT (1.13 g, 8.37 mmol), DIPEA (2.8 mL, 16.75 mmol) and methyl (S)-2-amino-3-((S)-2-oxopyrrolidin-3-yl)propanoate hydrochloride (amine fragment-2) (1.2 g, 6.70 mmol) at 0° C. simultaneously and stirred at room temperature for 16 h. The progress of the reaction was monitored by TLC and LCMS. After 16 h, reaction mixture was quenched with ice water (100 mL) and extracted with ethyl acetate (2×50 mL), combined organic layers were washed with brine solution (50 mL), dried over sodium sulfate and evaporated under reduced pressure. The crude residue was purified by normal phase chromatography by eluting 5% methanol in dichloromethane to afford tert-butyl 4-((((S)-1-(((S)-1-methoxy-1-oxo-3-((S)-2-oxopyrrolidin-3-yl)propan-2-yl)amino)-4-methyl-1-oxopentan-2-yl)carbamoyl)oxy)piperidine-1-carboxylate (5). TLC system: 10% Methanol in DCM R_{f}: 0.4 LCMS (ESI): m/z 527.74 [M+H]⁺

### Tert-butyl 4-((((S)-1-(((S)-1-hydroxy-3-((S)-2-oxopyrrolidin-3-yl) propan-2-yl) amino)-4-methyl-1-oxopentan-2-yl) carbamoyl) oxy) piperidine-1-carboxylate (6)

To a stirred solution of tert-butyl 4-((((S)-1-(((S)-1-methoxy-1-oxo-3-((S)-2-oxopyrrolidin-3-yl)propan-2-yl)amino)-4-methyl-1-oxopentan-2-yl)carbamoyl)oxy) piperidine-1-carboxylate (5) (620 mg, 1.17 mmol) in DCM (10 mL) was added 2M LiBH₄ in THF (1.2 mL, 2.35 mmol) at 0° C. and the reaction mixture stirred for 2 h at 0° C. The progress of the reaction was monitored by TLC and LCMS. Then reaction mixture was quenched with sat. NH₄Cl solution (30 mL) and extracted with ethyl acetate (2×30 mL). Organic layer was washed with brine solution (30 mL), dried over Na₂SO₄ and concentrated to get crude compound. It was purified by SFC prep purification to afford pure tert-butyl 4-((((S)-1-(((S)-1-hydroxy-3-((S)-2-oxopyrrolidin-3-yl) propan-2-yl) amino)-4-methyl-1-oxopentan-2-yl) carbamoyl) oxy) piperidine-1-carboxylate (6). TLC system: 10% Methanol in DCM R_{f}: 0.2 LCMS (ESI): m/z 499.70 [M+H]⁺

### Tert-butyl 4-((((S)-4-methyl-1-oxo-1-(((S)-1-oxo-3-((S)-2-oxopyrrolidin-3-yl) propan-2-yl) amino) pentan-2-yl) carbamoyl) oxy) piperidine-1-carboxylate (Compound C34)

To a stirred solution of tert-butyl 4-((((S)-1-(((S)-1-hydroxy-3-((S)-2-oxopyrrolidin-3-yl) propan-2-yl) amino)-4-methyl-1-oxopentan-2-yl) carbamoyl) oxy) piperidine-1-carboxylate (6) (100 mg, 0.20 mmol) was dissolved in dichloromethane (3 mL) was added Dess-Martin periodinane (255 mg, 0.60 mmol) at 0° C. and stirred at RT for 3 h. Reaction mixture was diluted with dichloromethane (10 mL) and washed with sat. Hypo solution (3×15 mL), sat. NaHCO₃ solution (3×15 mL). Organic layer was dried over anhydrous Na₂SO₄, filtered and concentrated to afford crude product, this residue was purified by prep HPLC chromatography to afford Tert-butyl 4-((((S)-4-methyl-1-oxo-1-(((S)-1-oxo-3-((S)-2-oxopyrrolidin-3-yl)propan-2-yl)amino)pentan-2-yl)carbamoyl)oxy)piperidine-1-carboxylate (Compound C34). TLC system: 10% Methanol in DCM Rf: 0.3 LCMS (ESI): m/z 497.44 (M+H)⁺

### (2S)-2-((S)-2-((((1-(tert-butoxycarbonyl) piperidin-4-yl) oxy) carbonyl) amino)-4-methylpentanamido)-1-hydroxy-3-((S)-2-oxopyrrolidin-3-yl) propane-1-sulfonate (Compound C51)

To a stirred solution of Tert-butyl 4-((((S)-4-methyl-1-oxo-1-(((S)-1-oxo-3-((S)-2-oxopyrrolidin-3-yl)propan-2-yl)amino)pentan-2-yl)carbamoyl)oxy)piperidine-1-carboxylate (Compound C34) (90 mg, 0.18 mmol) in ethanol (2 mL), EtOAc (1 mL), water (1 mL) was added NaHSO₃ (38 mg, 0.36 mmol) at RT and heated to 50° C. for 16 h. The progress of the reaction was monitored by TLC and LCMS. After 16 h, the reaction mixture was cooled to RT and filtered through celite pad then washed with ethanol (5 mL). Filtrate was evaporated under reduce pressure to afford crude residue. This residue was triturated with diethyl ether (2×5 mL), EtOAc (2×5 mL), the solvents were decanted, the solid was dried well to afford (2S)-2-((S)-2-((((1-(tert-butoxycarbonyl)piperidin-4-yl)oxy)carbonyl) amino)-4-methylpentanamido)-1-hydroxy-3-((S)-2-oxopyrrolidin-3-yl)propane-1-sulfonate (Compound C51). TLC system: 10% Methanol in DCM R_{f}: 0.1 LCMS (ESI): m/z 577.2 [M−H]− M=Free Base

### Example 35: Synthesis of Compounds C37 and C40

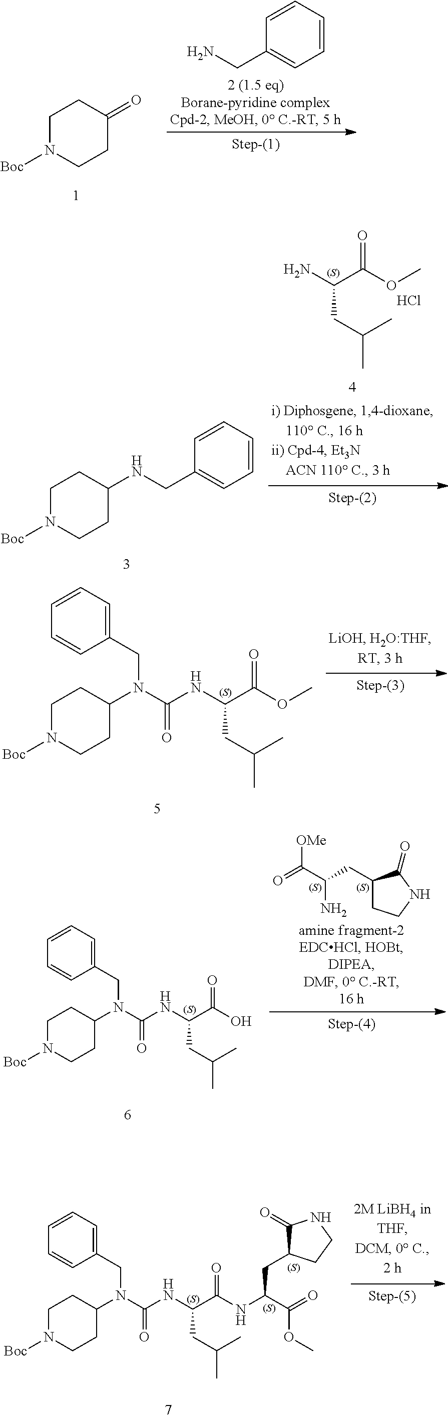

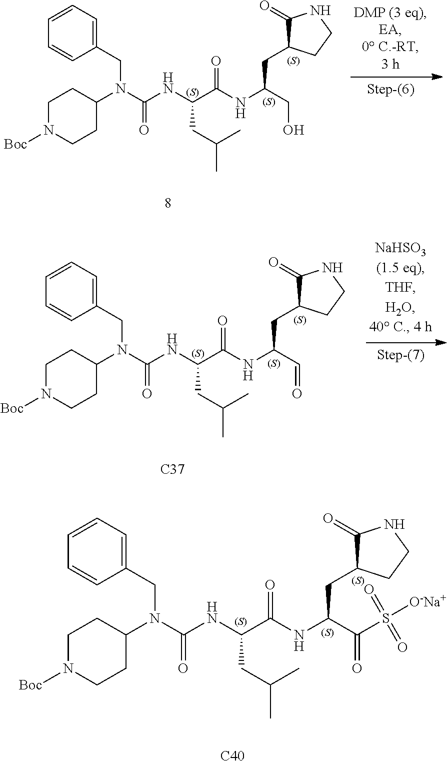

### Tert-butyl 4-(benzylamino)piperidine-1-carboxylate (3)

To a stirred solution of tert-butyl 4-oxopiperidine-1-carboxylate (1) (3 g, 15.0753 mmol) and phenylmethanamine (2) (2.2 mL, 18.0904 mmol) in Methanol (30 mL) added 8M Boran pyridine complex (2.8 mL, 3.39 mmol) at 0° C. and the reaction mixture stirred at RT for 16 h. The progress of the reaction was monitored by TLC. Reaction mixture was evaporated under reduced pressure to afford crude residue quenched with ice water (20 mL) and extracted with ethyl acetate (2×20 mL), combined organic layers were washed with brine solution (20 mL), dried over sodium sulfate and evaporated under reduced pressure. The crude residue was purified by normal phase chromatography to afford tert-butyl 4-(benzylamino)piperidine-1-carboxylate (3). TLC system: 50% Ethyl acetate in hexane Rf: 0.3 LCMS (ESI): m/z 291.52 [M+H]⁺

### Tert-butyl (S)-4-(1-benzyl-3-(1-methoxy-4-methyl-1-oxopentan-2-yl)ureido)piperidine-1-carboxylate (2)

To a stirred solution of methyl L-leucinate hydrochloride (4) (3 g, 13.574 mmol) in 1,4-dioxane (30 mL) added diphosgene (2.4 mL, 20.361 mmol) at RT and heated to reflux for 5 h. The progress of the reaction was monitored by TLC. Reaction mixture was evaporated under reduced pressure to afford crude residue, this residue was used directly in the subsequent reaction.

In another RB flask, tert-butyl 4-(benzylamino)piperidine-1-carboxylate (3) (3 g, 10.344 mmol) was taken in ACN (30 mL), and treated with triethylamine (4.47 mL, 31.034 mmol). The resulting reaction mixture was stirred for 5 min, then added above prepared reaction mass drop-wise and the reaction mixture stirred at RT and heated to 80° C. 16 h, the reaction mixture was quenched with ice water (20 mL) and extracted with ethyl acetate (2×20 mL), combined organic layers were washed with brine solution (20 mL), dried over sodium sulfate and evaporated under reduced pressure. The crude residue was purified by normal phase chromatography to afford tert-butyl (S)-4-(1-benzyl-3-(1-methoxy-4-methyl-1-oxopentan-2-yl)ureido)piperidine-1-carboxylate (5). TLC system: 50% Ethyl acetate in hexane Rf: 0.5 LCMS (ESI): m/z 484.50 [M+Na]⁺

### (benzyl(1-(tert-butoxycarbonyl)piperidin-4-yl)carbamoyl)-L-leucine (6)

To a stirred solution of tert-butyl (S)-4-(1-benzyl-3-(1-methoxy-4-methyl-1-oxopentan-2-yl)ureido)piperidine-1-carboxylate (5) (3.4 g, 7.375 mmol) in THF (30 mL), water (15 mL) was added lithium hydroxide (531 mg, 22.125 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×15 mL), dried over sodium sulfate, concentrated under reduced pressure to afford (benzyl(1-(tert-butoxycarbonyl)piperidin-4-yl)carbamoyl)-L-leucine (6). TLC system: 50% Ethyl acetate in hexane Rf: 0.1 LCMS (ESI): m/z 448.39 [M+H]⁺

### Tert-butyl 4-(1-benzyl-3-((S)-1-(((S)-1-methoxy-1-oxo-3-((S)-2-oxopyrrolidin-3-yl)propan-2-yl)amino)-4-methyl-1-oxopentan-2-yl)ureido)piperidine-1-carboxylate (7)

To a stirred solution of (benzyl(1-(tert-butoxycarbonyl)piperidin-4-yl)carbamoyl)-L-leucine (6) (2.3 g, 5.145 mmol) DMF (30 mL) added EDC.HCl (1.47 g, 7.718 mmol), HOBt (1.04 g, 7.718 mmol), DIPEA (2.8 mL, 15.436 mmol) and methyl (S)-2-amino-5-(2,3-dihydrobenzo[f][1,4]oxazepin-4(5H)-yl)-5-oxopentanoate hydrochloride (amine fragment-2) (1.14 g, 6.171 mmol) at 0° C. simultaneously and stirred at room temperature for 16 h. Reaction mixture was diluted with ice water (20 mL), extracted with ethyl acetate (2×20 mL), dried over sodium sulfate and evaporated under reduced pressure. The crude residue was purified by combi-flash NP, compound eluted at 2% methanol in dichloromethane to afford tert-butyl 4-(1-benzyl-3-((S)-1-(((S)-1-methoxy-1-oxo-3-((S)-2-oxopyrrolidin-3-yl)propan-2-yl)amino)-4-methyl-1-oxopentan-2-yl)ureido)piperidine-1-carboxylate (7). TLC system: 10% Methanol in dichloromethane Rf: 0.4 LCMS (ESI): m/z 616.88 [M+H]⁺

### Tert-butyl 4-(1-benzyl-3-((S)-1-(((S)-1-hydroxy-3-((S)-2-oxopyrrolidin-3-yl)propan-2-yl)amino)-4-methyl-1-oxopentan-2-yl)ureido)piperidine-1-carboxylate (8)

To a stirred solution of tert-butyl 4-(1-benzyl-3-((S)-1-(((S)-1-methoxy-1-oxo-3-((S)-2-oxopyrrolidin-3-yl)propan-2-yl)amino)-4-methyl-1-oxopentan-2-yl)ureido)piperidine-1-carboxylate (7) (1.3 g, 2.524 mmol) in THF (20 mL) was added 2M LiBH₄ in THF (2.5 mL, 5.048 mmol) at 0° C. and the reaction mixture stirred for 2 h at 0° C. The progress of the reaction was monitored by TLC and LCMS. Reaction mixture was quenched with sat. Ammonium chloride solution (20 mL) and extracted with ethyl acetate (2×20 mL). Organic layer was washed with brine solution (30 mL), dried over Na₂SO₄ and concentrated to get crude compound. It was purified by SFC prep purification to afford pure tert-butyl 4-(1-benzyl-3-((S)-1-(((S)-1-hydroxy-3-((S)-2-oxopyrrolidin-3-yl)propan-2-yl)amino)-4-methyl-1-oxopentan-2-yl)ureido)piperidine-1-carboxylate (8). TLC system: 10% Methanol in dichloromethane Rf: 0.3 LCMS (ESI): m/z 588.65 [M+H]⁺

### Tert-butyl 4-(1-benzyl-3-((S)-4-methyl-1-oxo-1-(((S)-1-oxo-3-((S)-2-oxopyrrolidin-3-yl)propan-2-yl)amino)pentan-2-yl)ureido)piperidine-1-carboxylate (Compound C37)

To a stirred solution of tert-butyl 4-(1-benzyl-3-((S)-1-(((S)-1-hydroxy-3-((S)-2-oxopyrrolidin-3-yl)propan-2-yl)amino)-4-methyl-1-oxopentan-2-yl)ureido)piperidine-1-carboxylate (8) (100 mg, 0.17 mmol) was dissolved in dichloromethane (10 mL) was added Dess-Martin periodinane (216 mg, 0.511 mmol) at 0° C. and stirred at RT for 3 h. Reaction mixture was diluted with DCM (15 mL) followed by sat. Hypo solution (3×15 mL), followed by sat. NaHCO₃ solution (3×15 mL). Organic layer was dried over anhydrous Na₂SO₄, filtered and concentrated to get crude compound. The crude compound was purified by prep HPLC to afford Tert-butyl 4-(1-benzyl-3-((S)-4-methyl-1-oxo-1-(((S)-1-oxo-3-((S)-2-oxopyrrolidin-3-yl)propan-2-yl)amino)pentan-2-yl)ureido)piperidine-1-carboxylate (Compound C37). TLC system: 10% Methanol in dichloromethane Rf: 0.5 LCMS (ESI): m/z 597.44 (M+H)⁺

### (2S)-2-((S)-2-(3-benzyl-3-(1-(tert-butoxycarbonyl)piperidin-4-yl)ureido)-4-methylpentanamido)-1-hydroxy-3-((S)-2-oxopyrrolidin-3-yl)propane-1-sulfonate (Compound C41)

To a stirred solution of Tert-butyl 4-(1-benzyl-3-((S)-4-methyl-1-oxo-1-(((S)-1-oxo-3-((S)-2-oxopyrrolidin-3-yl)propan-2-yl)amino)pentan-2-yl)ureido)piperidine-1-carboxylate (Compound C37) (50 mg, 1.103 mmol) in THF/H₂O (2/1) (3 mL) and NaHSO₃ (16 mg, 0.154 mmol) at RT and the RM was stirred at 40° C. for 16 h. The progress of the reaction was monitored by TLC and LCMS. Reaction mixture was filtered through celite pad and washed the celite pad with EtOH (20 mL) to afford crude compound. The crude was triturated with ether and n-pentane to afford sodium (2S)-2-((S)-2-(3-benzyl-3-(1-(tert-butoxycarbonyl)piperidin-4-yl)ureido)-4-methylpentanamido)-1-hydroxy-3-((S)-2-oxopyrrolidin-3-yl)propane-1-sulfonate (Compound C41). TLC system: 10% Methanol in dichloromethane Rf: 0.1 LCMS (ESI): m/z=586.60 [M−Sodium sulfonate]+

### Example 36: Synthesis of Compound C36

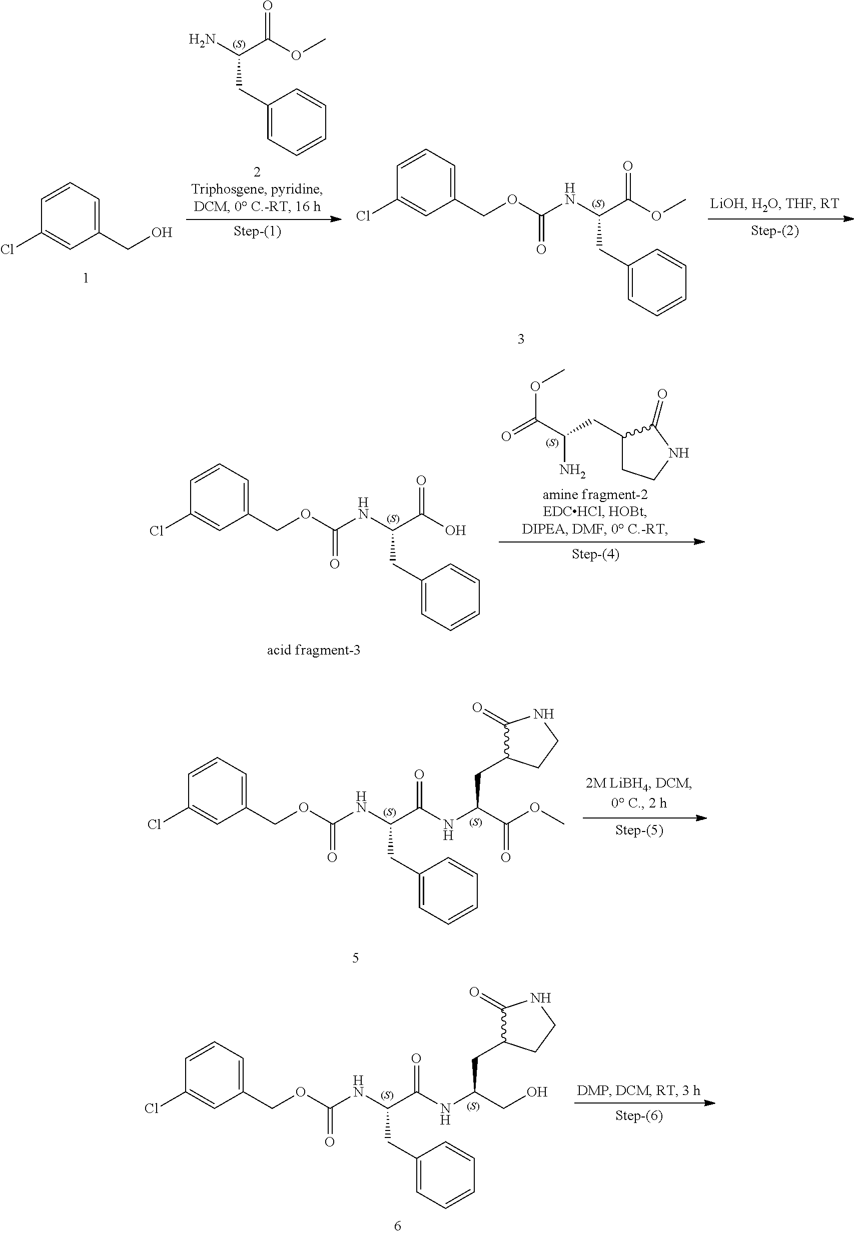

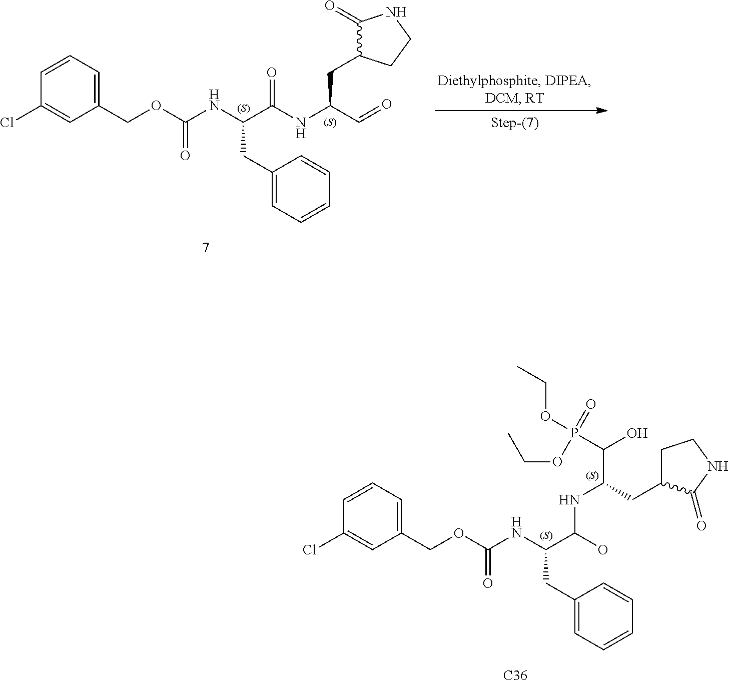

### Methyl (((3-chlorobenzyl)oxy)carbonyl)-L-phenylalaninate (3)

To a stirred solution of (3-chlorophenyl)methanol (1) (4.0 g, 2.8 mmol) in DCM (200 mL) was added pyridine (13.2 mL, 16.8 mmol), Triphosgene (4.14 g, 2.3 mmol) at 0° C. slowly, followed by methyl L-phenylalaninate (2) (6.02 g, 3.36 mmol) and stirred at RT for 16 h. The progress of the reaction was monitored by TLC and LCMS. Reaction mixture was quenched with ice water (150 mL), concentrated the organic layer, again washed with 1N HCl solution, then extracted with DCM (2×150 mL), dried over sodium sulfate and evaporated under reduced pressure. The crude residue was purified by silica gel column by eluting with 50% ethyl acetate in hexane to afford 1-methyl (((3-chlorobenzyl)oxy)carbonyl)-L-phenylalaninate (3). TLC system: 50% Ethyl acetate in Hexane Rf: 0.8 LCMS (ESI): m/z 348.29 [M+H]⁺

### (((3-chlorobenzyl)oxy)carbonyl)-L-phenylalanine (4)

To a stirred solution of 1-methyl (((3-chlorobenzyl)oxy)carbonyl)-L-phenylalaninate (3) (5.6 g, 16.13 mmol) in THF (60 mL), Methanol (20 mL) and DM water (40 mL), LiOH.H₂O (1.35 g, 32.26 mmol) was added. Reaction mixture was stirred at RT for 2 h. The progress of the reaction was monitored by TLC. After consumption of starting material, the reaction mixture was concentrated and acidified with 1N HCl, extracted with ethyl acetate (2×100 mL), dried over sodium sulfate and evaporated under reduced pressure to afford (((3-chlorobenzyl)oxy)carbonyl)-L-phenylalanine (4) which was used directly in the next step. TLC system: 50% Ethyl acetate in Hexane Rf: 0.2 LCMS (ESI): m/z 334.28 [M+H]⁺

### Methyl (2S)-2-((S)-2-((((3-chlorobenzyl)oxy)carbonyl)amino)-3-phenylpropanamido)-3-(2-oxopyrrolidin-3-yl)propanoate (5)

To a stirred solution of (((3-chlorobenzyl)oxy)carbonyl)-L-phenylalanine (4) (1.0 g, 3.003 mmol) (4) in DMF (100 mL) was added EDC.HCl (0.670 g, 3.603 mmol), HOBT (0.859 g, 4.50 mmol), DIPEA (1.65 mL, 9.00 mmol) and methyl (2S)-2-amino-3-(2-oxopyrrolidin-3-yl)propanoate (amine-fragment-2) (0.670 g, 3.60 mmol) at 0° C. simultaneously and stirred at room temperature for 16 h. Reaction mixture was diluted with ice water (100 mL), extracted with ethyl acetate (2×100 mL), dried over sodium sulfate and evaporated under reduced pressure. The crude residue was purified by silica gel column by eluting with 2% methanol in dichloromethane to afford methyl (2S)-2-((S)-2-((((3-chlorobenzyl)oxy)carbonyl)amino)-3-phenylpropanamido)-3-(2-oxopyrrolidin-3-yl)propanoate (5). TLC system: 10% Methanol in dichloromethane Rf: 0.5 LCMS (ESI): m/z 502.43 [M+H]⁺

### 3-Chlorobenzyl ((2S)-1-(((2S)-1-hydroxy-3-(2-oxopyrrolidin-3-yl)propan-2-yl)amino)-1-oxo-3-phenylpropan-2-yl)carbamate (6)

To a stirred solution of methyl (2S)-2-((S)-2-((((3-chlorobenzyl)oxy)carbonyl)amino)-3-phenylpropanamido)-3-(2-oxopyrrolidin-3-yl)propanoate (5) (0.75 g, 1.494 mmol) in Dichloromethane (50 mL), 2M LiBH₄ in THF (1.49 mL, 2.98 mmol) was added at 0° C. and stirred for 2 h The progress of the reaction was monitored by TLC and LCMS. the reaction mixture was quenched with saturated NH₄Cl solution and extracted with Dichloromethane (2×50 mL), filtrate was concentrated under reduced pressure to afford 3-chlorobenzyl ((2S)-1-(((2S)-1-hydroxy-3-(2-oxopyrrolidin-3-yl)propan-2-yl)amino)-1-oxo-3-phenylpropan-2-yl)carbamate (6). TLC system: 10% Methanol in dichloromethane Rf: 0.2 LCMS (ESI): m/z 474.4 [M+H]⁺

### 3-Chlorobenzyl ((2S)-1-oxo-1-(((2S)-1-oxo-3-(2-oxopyrrolidin-3-yl)propan-2-yl)amino)-3-phenylpropan-2-yl)carbamate (7)

To a stirred solution of (3-chlorobenzyl ((2S)-1-(((2S)-1-hydroxy-3-(2-oxopyrrolidin-3-yl)propan-2-yl)amino)-1-oxo-3-phenylpropan-2-yl)carbamate (6) (0.350 g, 0.738 mmol) was dissolved in dichloromethane (25 mL) was added Dess-Martin periodinane (0.626 g, 1.476 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude product, afford to 3-chlorobenzyl ((2S)-1-oxo-1-(((2S)-1-oxo-3-(2-oxopyrrolidin-3-yl)propan-2-yl)amino)-3-phenylpropan-2-yl)carbamate (7). TLC system: 10% Methanol in dichloromethane Rf: 0.3 LCMS (ESI): m/z 472.54 (M+H)⁺

### 3-Chlorobenzyl ((2S)-1-(((2S)-1-(diethoxyphosphoryl)-1-hydroxy-3-(2-oxopyrrolidin-3-yl)propan-2-yl)amino)-3-phenylpropan-2-yl)carbamate (Compound C36)

To a stirred solution of crude 3-chlorobenzyl ((2S)-1-oxo-1-(((2S)-1-oxo-3-(2-oxopyrrolidin-3-yl)propan-2-yl)amino)-3-phenylpropan-2-yl)carbamate (7) (330 mg, 0.7003 mmol) in DCM (20 mL) was added DIPEA (0.2 mL, 2.101 mmol) and diethylphosphate (0.3 mL, 2.101 mmol) at 0° C. and stirred at RT for 16 h. The progress of the reaction was monitored by TLC and LCMS. Reaction mixture was quenched with ice water (15 mL) extracted with DCM (3×15 mL). Organic layer was dried over anhydrous Na₂SO₄, filtered and dried well to afford crude compound. The crude compound was purified by prep HPLC to afford 3-Chlorobenzyl ((2S)-1-(((2S)-1-(diethoxyphosphoryl)-1-hydroxy-3-(2-oxopyrrolidin-3-yl)propan-2-yl)amino)-3-phenylpropan-2-yl)carbamate (Compound C36). TLC system: 10% Methanol in dichloromethane Rf: 0.4 LCMS (ESI): m/z 610.36 (M+H)⁺

### Example 37: Synthesis of Compounds C38 and C53

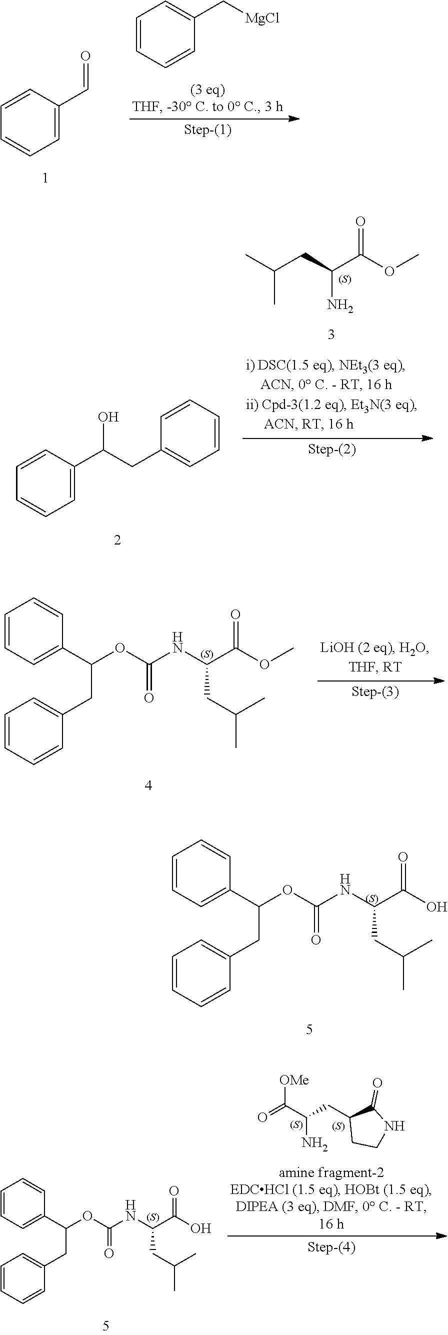

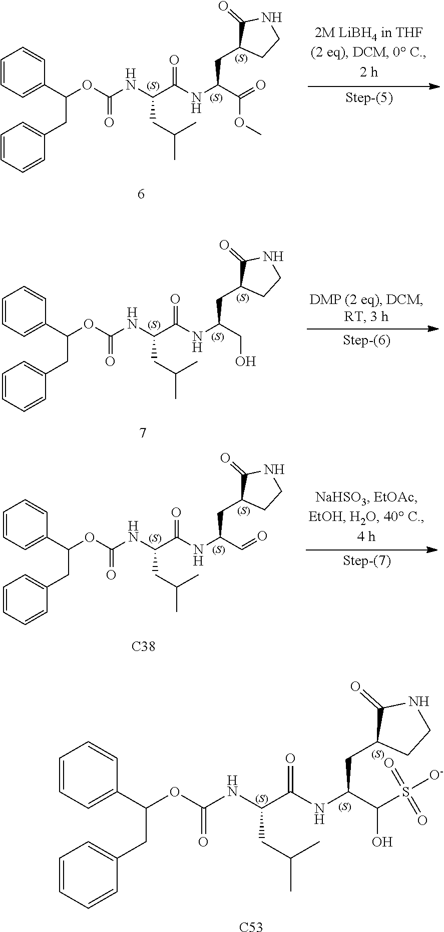

### 1,2-Diphenylethan-1-ol (2)

To a stirred solution of benzyl magnesium chloride (5.0 g, 46.7289 mmol) in THF (50 mL) was cooled to −30° C. then benzaldehyde (1) (70 mL, 140.1869 mmol) dissolved in THF (50 mL) added slowly and stirred at −30° C. to RT for 3 h. The progress of the reaction was monitored by TLC. Reaction mixture was quenched with saturated NH₄Cl solution (150 mL), and then extracted with ethyl acetate (2×100 mL), the organic layer was dried over sodium sulfate and evaporated under reduced pressure. The crude residue was purified by silica gel column by eluting with 20% ethyl acetate in hexane to afford 1,2-diphenylethan-1-ol (2). TLC system: 20% Ethyl acetate in Hexane Rf: 0.5 LCMS (ESI): m/z 183.46 [M−OH]⁺

### (((1-phenylpropan-2-yl)oxy)carbonyl)-L-leucine (4)

To a stirred solution of 1,2-diphenylethan-1-ol (2) (5.0 g, 25.25 mmol) in acetonitrile (250 mL), and then DSC (12.92 g, 50.4 mmol), Triethylamine (10.9 mL, 75.6 mmol) was added, Then the reaction mixture was stirred at RT for 16 h. Then methyl L-leucinate hydrochloride (3) (4.306 g, 29.6 mmol), triethylamine (10.6 mL, 72.0 mmol) was added, the reaction mixture was stirred at RT for 16 h, progress of the reaction was monitored by TLC. After consumption of starting material, the reaction mixture was concentrated and extracted with ethyl acetate (3×100 mL), dried over sodium sulfate and evaporated under reduced pressure, The crude residue was purified by silica gel column by eluting with 20% ethyl acetate in hexane to afford (((1-phenylpropan-2-yl)oxy)carbonyl)-L-leucine (4). TLC system: 20% Ethyl acetate in Hexane Rf: 0.8 LCMS (ESI): m/z 369.19 [M+Na]⁺

### ((1,2-Diphenylethoxy)carbonyl)-L-leucine (5)

To a stirred solution of (((1-phenylpropan-2-yl)oxy)carbonyl)-L-leucine (4) (5.8 g, 15.69 mmol) in THF (60 mL), and water (40 mL), LiOH.H₂O (1.976 g, 47.0 mmol) was added. Reaction mixture was stirred at RT for 2 h. The progress of the reaction was monitored by TLC. After consumption of starting material, the reaction mixture was concentrated and acidified with 1N HCl, extracted with ethyl acetate (3×100 mL), dried over sodium sulfate and evaporated under reduced pressure to afford ((1,2-diphenylethoxy)carbonyl)-L-leucine (5) which was used directly in the next step. TLC system: 50% Ethyl acetate in Hexane Rf: 0.1 LCMS (ESI): m/z 355.17 [M+Na]⁺

### Methyl (2S)-2-((2S)-2-(((1,2-diphenylethoxy)carbonyl)amino)-4-methylpentanamido)-3-((S)-2-oxopyrrolidin-3-yl)propanoate (6)

To a stirred solution of ((1,2-diphenylethoxy)carbonyl)-L-leucine (5) (3.0 g, 8.440 mmol) in DMF (30 mL) was added EDC.HCl (2.292 g, 12.00 mmol), HOBT (1.62 g, 12.00 mmol), DIPEA (4.64 mL, 25.2 mmol) was stirred at 0° C. for 10 minutes and then methyl (2S)-2-amino-3-(2-oxopyrrolidin-3-yl)propanoate (amine-fragment-2) (2.248 g, 10.12 mmol) was added at 0° C. simultaneously and stirred at room temperature for 16 h. Reaction mixture was diluted with ice water (100 mL), extracted with ethyl acetate (2×100 mL), dried over sodium sulfate and evaporated under reduced pressure. The crude residue was purified by silica gel column by eluting with 100% ethyl acetate to afford methyl (2S)-2-((2S)-2-(((1,2-diphenylethoxy)carbonyl)amino)-4-methylpentanamido)-3-((S)-2-oxopyrrolidin-3-yl)propanoate (6). TLC system: 100% Ethyl acetate Rf: 0.2 LCMS (ESI): m/z 524.55 [M+H]⁺

### 1,2-Diphenylethyl ((S)-1-(((S)-1-hydroxy-3-((S)-2-oxopyrrolidin-3-yl)propan-2-yl)amino)-4-methyl-1-oxopentan-2-yl)carbamate (7)

To a stirred solution of methyl (2S)-2-((2S)-2-(((1,2-diphenylethoxy)carbonyl)amino)-4-methylpentanamido)-3-((S)-2-oxopyrrolidin-3-yl)propanoate (6) (0.530 g, 1.013 mmol) in DCM (50 mL), 2M LiBH₄ in THF (1.013 mL, 2.026 mmol) was added at 0° C. and stirred for 2 h. The progress of the reaction was monitored by TLC and LCMS. The reaction mixture was quenched with saturated NH₄Cl solution (10 mL) and extracted with DCM (2×50 mL), filtrate was concentrated under reduced pressure to afford 1,2-diphenylethyl ((S)-1-(((S)-1-hydroxy-3-((S)-2-oxopyrrolidin-3-yl)propan-2-yl)amino)-4-methyl-1-oxopentan-2-yl)carbamate (7) TLC system: 5% Methanol in dichloromethane Rf: 0.4 LCMS (ESI): m/z 496.59 (M+H)⁺

### 1,2-Diphenylethyl ((S)-4-methyl-1-oxo-1-(((S)-1-oxo-3-((S)-2-oxopyrrolidin-3-yl)propan-2-yl)amino)pentan-2-yl)carbamate (Compound C38)

To a stirred solution of 1,2-diphenylethyl ((S)-1-(((S)-1-hydroxy-3-((S)-2-oxopyrrolidin-3-yl)propan-2-yl)amino)-4-methyl-1-oxopentan-2-yl)carbamate (7) (0.300 mg, 0.605 mmol) was dissolved in dichloromethane (20 mL) was added Dess-Martin periodinane (0.513 g, 1.216 mmol) at 0° C. and stirred at RT for 3 h. Reaction mixture was diluted with DCM (20 mL) and organic layer was washed with sat. Hypo solution (3×20 mL), sat. NaHCO₃ solution (3×20 mL). Organic layer was dried over anhydrous Na₂SO₄, filtered and concentrated and purified by PHPLC to afford 1,2-diphenylethyl ((S)-4-methyl-1-oxo-1-(((S)-1-oxo-3-((S)-2-oxopyrrolidin-3-yl)propan-2-yl)amino)pentan-2-yl)carbamate (Compound C38). TLC system: 10% Methanol in dichloromethane Rf: 0.5 LCMS (ESI): m/z 494.44 (M+H)⁺

### (2S)-2-((2S)-2-(((1,2-Diphenylethoxy)carbonyl)amino)-4-methylpentanamido)-1-hydroxy-3-((S)-2-oxopyrrolidin-3-yl)propane-1-sulfonate (Compound C53)

To a stirred solution of 1,2-diphenylethyl ((S)-4-methyl-1-oxo-1-(((S)-1-oxo-3-((S)-2-oxopyrrolidin-3-yl)propan-2-yl)amino)pentan-2-yl)carbamate (Compound C38) (0.100 mg, 0.2027 mmol) was dissolved in ethyl acetate (2.0 mL), ethanol (1.0 mL), water (0.4 mL), and then NaHSO₃ (0.0632 mg, 0.6081 mmol) was added and stirred at 40° C. for 4 h. The progress of the reaction was monitored by TLC and LCMS. The reaction mixture was filtered through celite pad and filtrate was dried over sodium sulphate, concentrated and the crude compound was washed with diethyl ether to afford (2S)-2-((2S)-2-(((1,2-diphenylethoxy)carbonyl)amino)-4-methylpentanamido)-1-hydroxy-3-((S)-2-oxopyrrolidin-3-yl)propane-1-sulfonate (Compound C53). TLC system: 10% Methanol in dichloromethane Rf: 0.2 LCMS (ESI): m/z 574.2 (M+H)⁺

### Example 38: Synthesis of Compounds C39 and C50

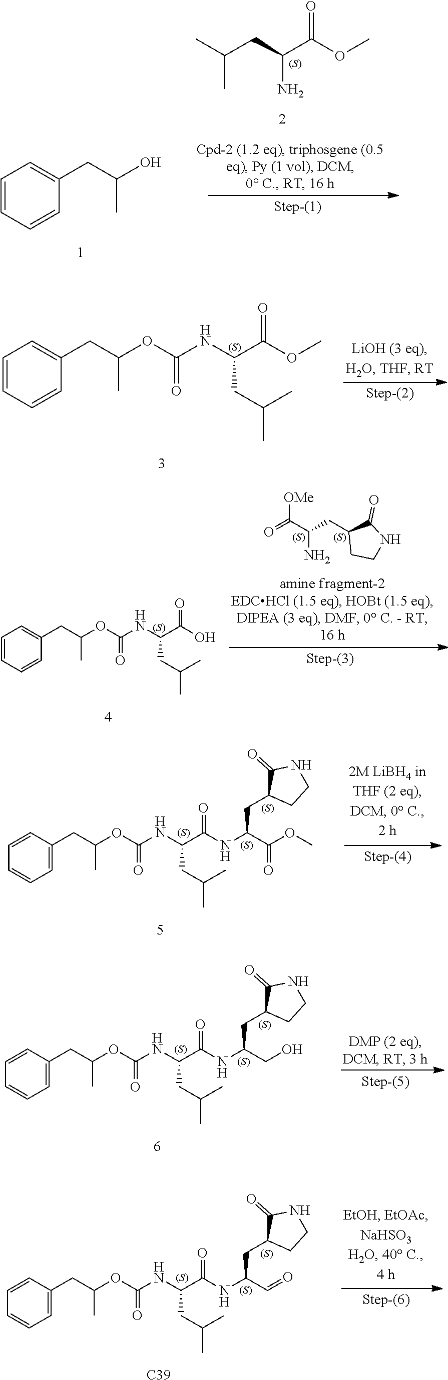

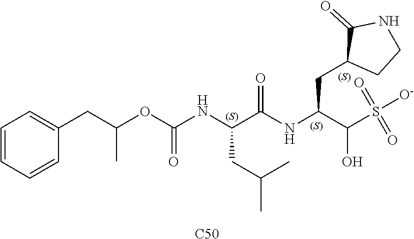

### Methyl (((1-phenylpropan-2-yl)oxy)carbonyl)-L-leucinate (3)

To a stirred solution of 1-phenylpropan-2-ol (1) (7.0 g, 51.39 mmol) in DCM (35 mL) was added pyridine (6.0 mL, 77.09 mmol), Triphosgene (7.62 g, 25.6 mmol) at 0° C. slowly, followed by methyl L-leucinate hydrochloride (2) (8.942 g, 61.6 mmol) and stirred at RT for 16 h. The progress of the reaction was monitored by TLC and LCMS. Reaction mixture was quenched with ice water (150 mL), concentrated the organic layer, again washed with 1N HCl solution, then extracted with DCM (2×150 mL), dried over sodium sulfate and evaporated under reduced pressure. The crude residue was purified by silica gel column by eluting with 20% ethyl acetate in hexane to afford methyl (((1-phenylpropan-2-yl)oxy)carbonyl)-L-leucinate (3). TLC system: 20% Ethyl acetate in Hexane Rf: 0.8 LCMS (ESI): m/z 308.52 [M+H]⁺

### (((1-phenylpropan-2-yl)oxy)carbonyl)-L-leucine (4)

To a stirred solution of methyl (((1-phenylpropan-2-yl)oxy)carbonyl)-L-leucinate (3) (6.7 g, 21.81 mmol) in THF (60 mL), and DM water (40 mL), LiOH.H₂O (2.74 g, 65.43 mmol) was added. Reaction mixture was stirred at RT for 2 h. The progress of the reaction was monitored by TLC and LCMS. After consumption of starting material, the reaction mixture was concentrated and acidified with 1N HCL, extracted with ethyl acetate (3×100 mL), dried over sodium sulfate and evaporated under reduced pressure to afford (((1-phenylpropan-2-yl)oxy)carbonyl)-L-leucine (4) which was used directly in the next step. TLC system: 50% Ethyl acetate in Hexane Rf: 0.1 LCMS (ESI): m/z 294.55 [M+H]⁺

### Methyl (2S)-2-((2S)-4-methyl-2-((((1-phenylpropan-2-yl)oxy)carbonyl)amino)pentanamido)-3-((S)-2-oxopyrrolidin-3-yl)propanoate (5)

To a stirred solution of (((1-phenylpropan-2-yl)oxy)carbonyl)-L-leucine (4) (2.5 g, 8.532 mmol) in DMF (25 mL) was added EDC.HCl (1.518 g, 10.20 mmol), HOBT (1.073 g, 7.95 mmol), DIPEA (4.715 mL, 25.5 mmol) was stirred at 0° C. for 10 minutes and then methyl (2S)-2-amino-3-(2-oxopyrrolidin-3-yl)propanoate (amine-fragment-2) (2.273 g, 10.2 mmol) was added at 0° C. simultaneously and stirred at room temperature for 16 h. Reaction mixture was diluted with ice water (100 mL), extracted with ethyl acetate (2×100 mL), dried over sodium sulfate and evaporated under reduced pressure. The crude residue was purified by silica gel column by eluting with 100% ethyl acetate to afford methyl (2S)-2-((2S)-4-methyl-2-((((1-phenylpropan-2-yl)oxy)carbonyl)amino)pentanamido)-3-((S)-2-oxopyrrolidin-3-yl)propanoate (5). TLC system: 100% Ethyl acetate Rf: 0.2 LCMS (ESI): m/z 462.65 [M+H]⁺

### 1-Phenylpropan-2-yl ((S)-1-(((S)-1-hydroxy-3-((S)-2-oxopyrrolidin-3-yl)propan-2-yl)amino)-4-methyl-1-oxopentan-2-yl)carbamate (6)

To a stirred solution of methyl (2S)-2-((2S)-4-methyl-2-((((1-phenylpropan-2-yl)oxy)carbonyl)amino)pentanamido)-3-((S)-2-oxopyrrolidin-3-yl)propanoate (5) (2.4 g, 5.199 mmol) in Dichloromethane (50 mL), 2M LiBH₄ in THF (5.2 mL, 10.39 mmol) was added at 0° C. and stirred for 2 h. The progress of the reaction was monitored by TLC and LCMS. The reaction mixture was quenched with saturated NH₄Cl (20 mL) solution and extracted with Dichloromethane (2×50 mL), filtrate was concentrated under reduced pressure to afford 1-phenylpropan-2-yl ((S)-1-(((S)-1-hydroxy-3-((S)-2-oxopyrrolidin-3-yl)propan-2-yl)amino)-4-methyl-1-oxopentan-2-yl)carbamate (6). TLC system: 100% Ethyl acetate Rf: 0.1 LCMS (ESI): m/z 434.5 [M+H]⁺

### 1-Phenylpropan-2-yl ((S)-4-methyl-1-oxo-1-(((S)-1-oxo-3-((S)-2-oxopyrrolidin-3-yl)propan-2-yl)amino)pentan-2-yl)carbamate (Compound C39)

To a stirred solution of 1-phenylpropan-2-yl ((S)-1-(((S)-1-hydroxy-3-((S)-2-oxopyrrolidin-3-yl)propan-2-yl)amino)-4-methyl-1-oxopentan-2-yl)carbamate (6) (0.300 g, 0.461 mmol) was dissolved in dichloromethane (20 mL) was added Dess-Martin periodinane (0.390 g mg, 0.923 mmol) at 0° C. and stirred at RT for 3 h. The progress of the reaction was monitored by TLC and LCMS. After 3 h, the reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude product, afford to 1-phenylpropan-2-yl ((S)-4-methyl-1-oxo-1-(((S)-1-oxo-3-((S)-2-oxopyrrolidin-3-yl)propan-2-yl)amino)pentan-2-yl)carbamate (Compound C39). TLC system: 5% Methanol in dichloromethane Rf: 0.8 LCMS (ESI): m/z 432.58 (M+H)⁺

### (2S)-1-Hydroxy-2-((2S)-4-methyl-2-((((1-phenylpropan-2-yl)oxy)carbonyl)amino)pentanamido)-3-((S)-2-oxopyrrolidin-3-yl)propane-1-sulfonate (Compound C50)

To a stirred solution of 1-phenylpropan-2-yl ((S)-4-methyl-1-oxo-1-(((S)-1-oxo-3-((S)-2-oxopyrrolidin-3-yl)propan-2-yl)amino)pentan-2-yl)carbamate (Compound C39) (0.100 g, 0.231 mmol) was dissolved in Ethyl acetate (2.0 mL), Ethanol (1.0 mL), Water (0.4 mL), and then NaHSO₃ (0.0361 mg, 0.347 mmol) was added and stirred at 40° C. for 16 h. The progress of the reaction was monitored by TLC and LCMS. The reaction mixture was filtered through celite pad and filtrate was dried over sodium sulphate, concentrated and the crude compound was washed with diethyl ether to afford (2S)-1-hydroxy-2-((2S)-4-methyl-2-((((1-phenylpropan-2-yl)oxy)carbonyl)amino)pentanamido)-3-((S)-2-oxopyrrolidin-3-yl)propane-1-sulfonate (Compound C50). TLC system: 15% Methanol in dichloromethane Rf: 0.2 LCMS (ESI): m/z 512.12 (M+H)⁺

### Example 39: Synthesis of Compounds C52 and C41

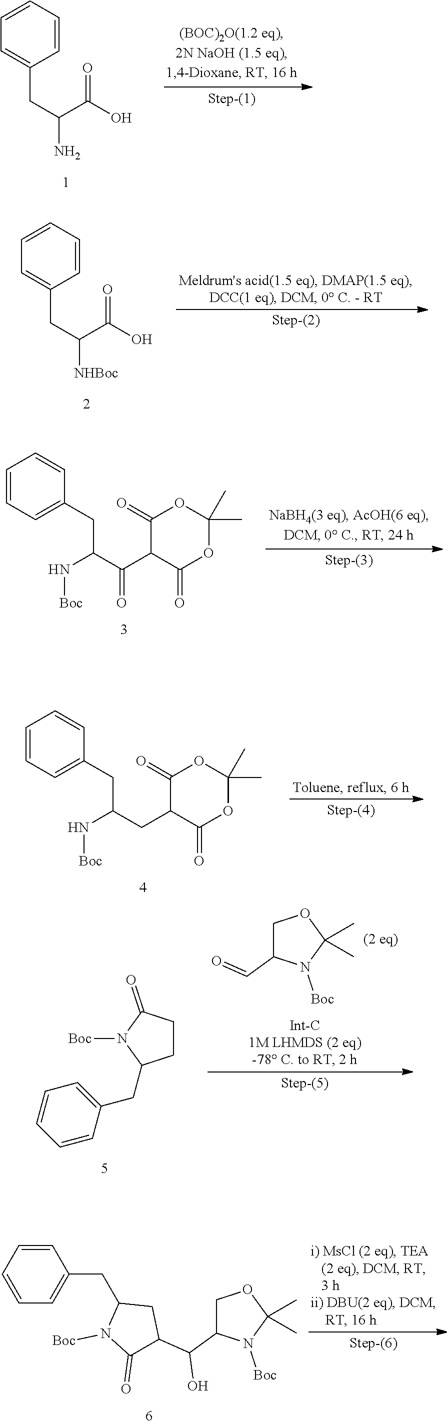

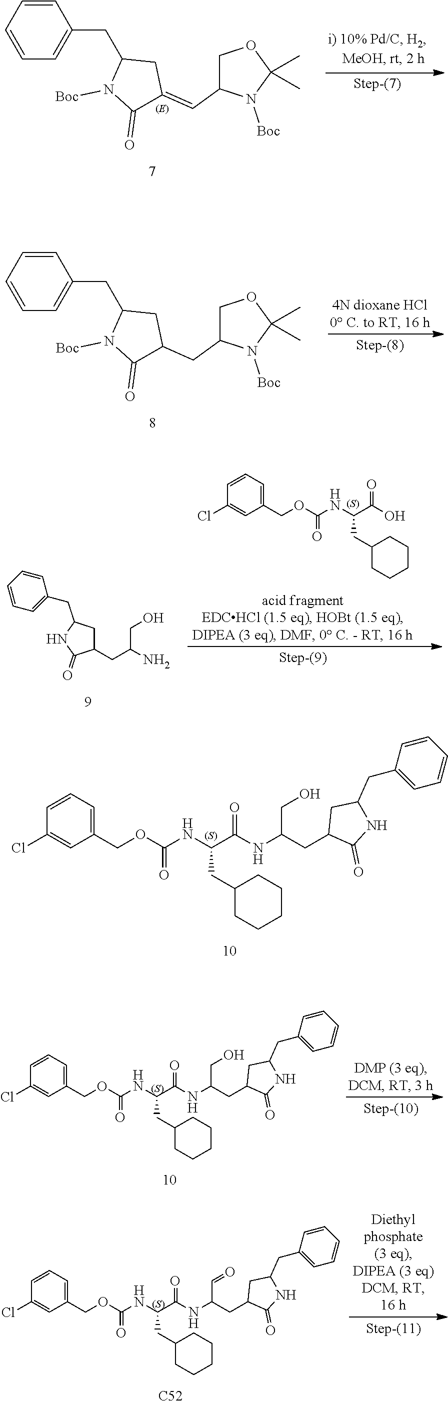

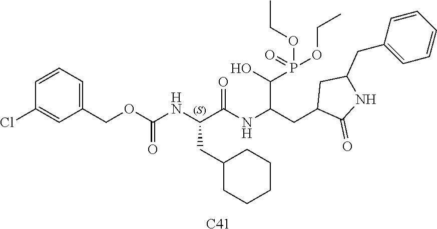

### (Tert-butoxycarbonyl)phenylalanine (2)

To a stirred solution of phenylalanine (1) (10 g, 60.606 mmol) in 1,4-Dioxane (100 mL) was added 2N NaOH solution (36 mL, 72.72 mmol) and followed by added Boc anhydride (14.5 mL, 66.66 mmol) at 0° C. then the reaction mixture allowed to RT for 24 h. The progress of the reaction was monitored by TLC and LCMS. Reaction mixture was evaporated under reduced pressure to afford crude, this crude was diluted with water and acidified pH˜2 with 2N HCl and extracted with DCM (2×100 mL), dried over Na₂SO₄ and concentrated to afforded (tert-butoxycarbonyl)phenylalanine (2) TLC system: 50% Ethyl acetate in Pet ether Rf: 0.7 LCMS (ESI): m/z=288.20[M+Na]⁺

### Tert-butyl (1-(2,2-dimethyl-4,6-dioxo-1,3-dioxan-5-yl)-1-oxo-3-phenylpropan-2-yl)carbamate (3)

To a stirred solution of (tert-butoxycarbonyl) phenylalanine (2) (15 g, 56.60 mmol) in DCM (150 mL) was added DMAP (10.35 g, 84.905 mmol) and followed by added Meldrum's acid (12.2 g, 84.905 mmol) portion wise at 0° C. after stirred for 15 min added DCC in DCM (11.6 g, 56.6037 mmol) slowly at 0° C. and then reaction mixture allowed to RT for 24 h. The progress of the reaction was monitored by TLC and LCMS, reaction mixture quenched with ice water (200 mL) and extracted with DCM (2×100 mL), dried over Na₂SO₄ and concentrated to get crude compound, this crude was purified by normal phase chromatography with eluted 80% ethyl acetate in hexane to afford tert-butyl (1-(2,2-dimethyl-4,6-dioxo-1,3-dioxan-5-yl)-1-oxo-3-phenylpropan-2-yl)carbamate (3) TLC system: 50% Ethyl acetate in Pet ether Rf: 0.2 LCMS (ESI): m/z=390.64[M−H]⁺

### Tert-butyl (1-(2,2-dimethyl-4,6-dioxo-1,3-dioxan-5-yl)-3-phenylpropan-2-yl)carbamate (4)

To a stirred solution of tert-butyl (1-(2,2-dimethyl-4,6-dioxo-1,3-dioxan-5-yl)-1-oxo-3-phenylpropan-2-yl)carbamate (3) (10.0 g, 25.575 mmol) in DCM (100 mL) added AcOH (9.2 mL, 153.452 mmol) and followed by added NaBH₄ (3.9 g, 102.3017 mmol) slowly portion wise for 30 min at 0° C. then reaction mixture stirred at room temperature for 16 h. The progress of the reaction was monitored by TLC and LCMS. After 16 h, reaction mixture was quenched with ice water (200 mL) and extracted with DCM (2×100 mL), dried over Na₂SO₄ and concentrated to get crude compound, this crude was purified by normal phase chromatography with eluted 40% ethyl acetate in hexane to afford tert-butyl (1-(2,2-dimethyl-4,6-dioxo-1,3-dioxan-5-yl)-3-phenylpropan-2-yl)carbamate (4). TLC system: 50% Ethyl acetate in Pet ether Rf: 0.3 LCMS (ESI): m/z=376.39 [M−H]⁺

### Tert-butyl 2-benzyl-5-oxopyrrolidine-1-carboxylate (5)

To a stirred solution of tert-butyl (1-(2,2-dimethyl-4,6-dioxo-1,3-dioxan-5-yl)-3-phenylpropan-2-yl)carbamate (4) (12 g, 31.8302 mmol) in toluene (240 mL) then reaction mixture heated to reflux for 6 h. The progress of the reaction was monitored by TLC. After consumption of starting material, the reaction mixture was evaporated under reduced pressure to obtained crude compound, this crude was purified by normal phase chromatography with eluted 20% ethyl acetate in hexane to afford tert-butyl 2-benzyl-5-oxopyrrolidine-1-carboxylate (5). TLC system: 50% Ethyl acetate in Pet ether Rf: 0.7 LCMS (ESI): m/z=298.26 [M+Na]⁺

### Tert-butyl 4-((5-benzyl-1-(tert-butoxycarbonyl)-2-oxopyrrolidin-3-yl)(hydroxy)methyl)-2,2-dimethyloxazolidine-3-carboxylate (6)

To a stirred solution of tert-butyl 2-benzyl-5-oxopyrrolidine-1-carboxylate (5) (4 g, 14.545 mmol) in THF (40 mL) at −78° C. added 1M LiHMDS in THF (29 mL, 29.0909 mmol) and stirred at same temperature for 30 min., then added Int-C (5 g, 21.818 mmol) and stirred at same temperature for 2 h. The progress of the reaction was monitored by TLC and LCMS. Reaction mixture was quenched with NH₄Cl solution (100 mL) and extracted with EtOAc (3×300 mL), dried over Na₂SO₄ and evaporated under reduced pressure afford crude tert-butyl 4-((5-benzyl-1-(tert-butoxycarbonyl)-2-oxopyrrolidin-3-yl)(hydroxy)methyl)-2,2-dimethyloxazolidine-3-carboxylate (6) (which was used directly in the next step. TLC system: 30% EtOAc in pet ether R_{f}: 0.4 LCMS (ESI): m/z 427.41 (M+Na)⁺

### Tert-butyl (E)-4-((5-benzyl-1-(tert-butoxycarbonyl)-2-oxopyrrolidin-3-ylidene)methyl)-2,2-dimethyloxazolidine-3-carboxylate (7)

To a stirred solution Tert-butyl(4R)-4-((1S)-(1-(tert-butoxycarbonyl)-2-oxopyrrolidin-3-yl) (hydroxy) methyl)-2,2-dimethyloxazolidine-3-carboxylate (6) (6 g crude, 11.9047 mmol) in DCM (60 mL) at 0° C. was added NEt₃ (5.1 mL) and MsCl (4.1 g, 35.714 mmol) slowly at same temperature reaction mixture was stirred at room temperature for 16 h. The progress of the reaction was monitored by TLC and LCMS. Reaction mixture was quenched with NH₄Cl solution (100 mL) and extracted with DCM (3×300 mL), dried over Na₂SO₄ and evaporated under reduced pressure afford tert-butyl 4-((5-benzyl-1-(tert-butoxycarbonyl)-2-oxopyrrolidin-3-yl)((methylsulfonyl)oxy)methyl)-2,2-dimethyloxazolidine-3-carboxylate (6.5 g, crude). This crude compound was dissolved in DCM (60 mL) then added DBU (5 mL, 33.505 mmol) at 0° C. and stirred for 3 h at RT. The progress of the reaction was monitored by TLC and LCMS. Reaction mixture was quenched with ice water (100 mL) extracted with DCM (2×100 mL), dried over anhydrous Na₂SO₄ and evaporated under reduced pressure. The crude residue was purified by normal phase chromatography with eluted 20% ethyl acetate in hexane to afford Tert-butyl (E)-4-((5-benzyl-1-(tert-butoxycarbonyl)-2-oxopyrrolidin-3-ylidene)methyl)-2,2-dimethyloxazolidine-3-carboxylate (7). TLC system: 10% Ethyl acetate in Pet ether Rf: 0.7 LCMS (ESI): m/z=509.64 [M+Na]⁺

### Tert-butyl 4-((5-benzyl-1-(tert-butoxycarbonyl)-2-oxopyrrolidin-3-yl)methyl)-2,2-dimethyloxazolidine-3-carboxylate (8)

To a stirred solution of tert-butyl (E)-4-((5-benzyl-1-(tert-butoxycarbonyl)-2-oxopyrrolidin-3-ylidene)methyl)-2,2-dimethyloxazolidine-3-carboxylate (7) (1.5 g, 3.0864 mmol) in MeOH (20 mL) was added 10% Pd/C (800 mg, 10% by wet) at RT and the reaction mixture was stirred at RT for 2 h under H₂ gas balloon pressure. The progress of the reaction was monitored by TLC and LCMS. After 3 h, reaction mixture was filtered through celite and evaporated under reduced pressure. The crude residue was purified by normal phase chromatography with eluted 10% ethyl acetate in hexane to afford tert-butyl 4-((5-benzyl-1-(tert-butoxycarbonyl)-2-oxopyrrolidin-3-yl)methyl)-2,2-dimethyloxazolidine-3-carboxylate (8). TLC system: 10% EtOAc in pet ether R_{f}: 0.4

### 3-(2-amino-3-hydroxypropyl)-5-benzylpyrrolidin-2-one (9)

To a stirred solution of tert-butyl 4-((5-benzyl-1-(tert-butoxycarbonyl)-2-oxopyrrolidin-3-yl)methyl)-2,2-dimethyloxazolidine-3-carboxylate (8) (1.1 g, 2.254 mmol mmol) in dioxane (10 mL) was added 4M HCl in dioxane (10 mL) at 0° C. The mixture was allowed to RT and stirred for 16 h. After consumption of starting material, the solvent was evaporated to afford crude residue. It was triturated with diethyl ether to afford 3-(2-amino-3-hydroxypropyl)-5-benzylpyrrolidin-2-one (9). TLC system: 10% MeOH in DCM R_{f}: 0.1 LCMS (ESI): m/z 249.43 (M+H)⁺

### 3-Chlorobenzyl ((2S)-1-((1-(5-benzyl-2-oxopyrrolidin-3-yl)-3-hydroxypropan-2-yl)amino)-3-cyclohexyl-1-oxopropan-2-yl)carbamate (10)

To a stirred solution of (S)-2-((((3-chlorobenzyl)oxy)carbonyl)amino)-3-cyclohexylpropanoic acid (amine fragment) (200 mg, 0.589 mmol) DMF (10 mL) added EDC.HCl (170 mg, 0.884 mmol), HOBT (120 mg, 0.884 mmol), DIPEA (0.3 mL, 1.769 mmol) and added 3-(2-amino-3-hydroxypropyl)-5-benzylpyrrolidin-2-one (9) (175 mg, 0.707 mmol) at 0° C. simultaneously and stirred at room temperature for 16 h. The progress of the reaction was monitored by TLC and LCMS. After 16 h, Reaction mixture was quenched with ice water (20 mL) extracted with DCM (2×50 mL), dried over anhydrous Na₂SO₄ and evaporated under reduced pressure. The crude residue was purified by normal phase chromatography with eluted 20% methanol in DCM to afford 3-chlorobenzyl ((2S)-1-((1-(5-benzyl-2-oxopyrrolidin-3-yl)-3-hydroxypropan-2-yl)amino)-3-cyclohexyl-1-oxopropan-2-yl)carbamate (10). TLC system: 10% Methanol in DCM R_{f}: 0.4 LCMS (ESI): m/z 570.70 [M+H]⁺

### 3-Chlorobenzyl ((2S)-1-((1-(5-benzyl-2-oxopyrrolidin-3-yl)-3-oxopropan-2-yl)amino)-3-cyclohexyl-1-oxopropan-2-yl)carbamate (Compound C52)

To a stirred solution of 3-chlorobenzyl ((2S)-1-((1-(5-benzyl-2-oxopyrrolidin-3-yl)-3-hydroxypropan-2-yl)amino)-3-cyclohexyl-1-oxopropan-2-yl)carbamate (10) (150 mg, 0.263 mmol) was dissolved in dichloromethane (10 mL) was added Dess-Martin periodinane (335 mg, 0.7908 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude residue. It was purified by prep HPLC to afford 3-chlorobenzyl ((2S)-1-((1-(5-benzyl-2-oxopyrrolidin-3-yl)-3-oxopropan-2-yl)amino)-3-cyclohexyl-1-oxopropan-2-yl)carbamate (Compound C52). TLC system: 10% Methanol in DCM R_{f}: 0.5 LCMS (ESI): m/z 568.53 [M+H]⁺

### 3-Chlorobenzyl ((2S)-1-((3-(5-benzyl-2-oxopyrrolidin-3-yl)-1-(diethoxyphosphoryl)-1-hydroxypropan-2-yl)amino)-3-cyclohexyl-1-oxopropan-2-yl)carbamate (Compound C41)

To a stirred solution of 3-chlorobenzyl ((2S)-1-((1-(5-benzyl-2-oxopyrrolidin-3-yl)-3-oxopropan-2-yl)amino)-3-cyclohexyl-1-oxopropan-2-yl)carbamate (Compound C52) (150 mg crude, 0.264 mmol) in DCM (10 mL) added DIPEA (0.15 mL, 0.793 mmol) followed by added diethyl phosphite (0.12 mL, 0.793 mmol) and the reaction mixture stirred at RT for 16 h. The progress of the reaction was monitored by TLC and LCMS. After 16 h, the reaction mixture quenched with Sat. ammonium chloride (20 mL) and extracted with DCM (2×20 mL). Combined organic layer was dried over anhydrous Na₂SO₄, and evaporated to afford crude residue. It was purified by prep HPLC to afford 3-chlorobenzyl ((2S)-1-((3-(5-benzyl-2-oxopyrrolidin-3-yl)-1-(diethoxyphosphoryl)-1-hydroxypropan-2-yl)amino)-3-cyclohexyl-1-oxopropan-2-yl)carbamate (Compound C41). TLC system: 5% Methanol in DCM R_{f}: 0.4 LCMS (ESI): m/z 706.74 [M+H]⁺

### Example 40: Synthesis of Compounds C42 and C49

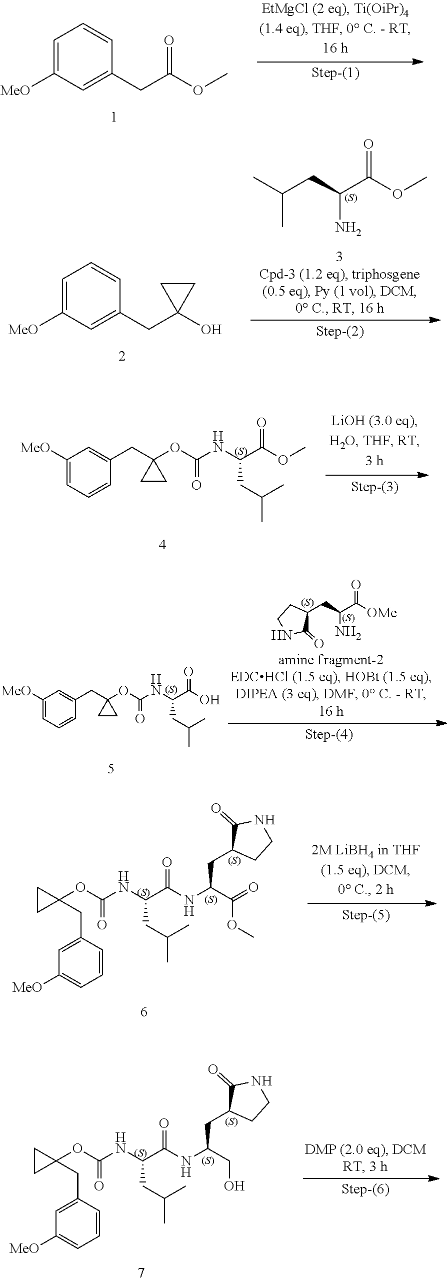

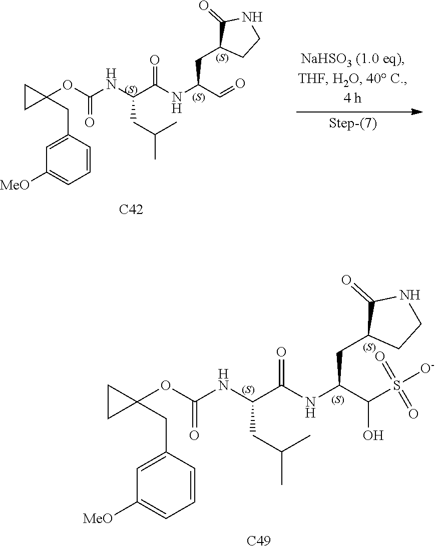

### 1-(3-methoxybenzyl)cyclopropan-1-ol (2)

Solution of methyl 2-(3-methoxyphenyl)acetate (10.0 g, 55.49 mmol) in THF (100 mL) was cooled to 0° C. then Ti(OiPr)₄ (22.06 mL, 77.69 mmol) and ethyl magnesium chloride (138.0 mL, 138.73 mmol) added slowly and stirred at RT for 3 h. The progress of the reaction was monitored by TLC. Reaction mixture was quenched with saturated NH₄Cl solution (150 mL), and filtered through celite pad and then extracted with ethyl acetate (2×100 mL), the combined organic layer was dried over sodium sulfate and evaporated under reduced pressure. The crude residue was purified by silica gel column by eluting with 20% ethyl acetate in hexane to afford 1-(3-methoxybenzyl)cyclopropan-1-ol (2). TLC system: 20% Ethyl acetate in Hexane Rf: 0.4 LCMS ESI): m/z 179.35[M+H]⁺

### Methyl ((1-(3-methoxybenzyl)cyclopropoxy)carbonyl)-L-leucinate (4)

To a stirred solution of 1-(3-methoxybenzyl)cyclopropan-1-ol (2) (5.6 g, 37.00 mmol) in DCM (100 mL) was added pyridine (6.0 mL, 77.09 mmol), Triphosgene (5.47 g, 18.5 mmol) slowly at 0° C., followed by methyl L-leucinate hydrochloride (3) (6.49 g, 44.00 mmol) and stirred at RT for 16 h. The progress of the reaction was monitored by TLC. Reaction mixture was quenched with ice water (150 mL), then added DCM (300 mL) and washed with 1N HCl solution. The organic layer was dried over sodium sulfate and evaporated under reduced pressure. The crude residue was purified by silica gel column by eluting with 20% ethyl acetate in hexane to afford methyl ((1-(3-methoxybenzyl)cyclopropoxy)carbonyl)-L-leucinate (4). TLC system: 20% Ethyl acetate in Pet ether Rf: 0.8 LCMS (ESI): m/z 350.47 [M+H]⁺

### ((1-(3-methoxybenzyl)cyclopropoxy)carbonyl)-L-leucine (5)

To a stirred solution of methyl ((1-(3-methoxybenzyl)cyclopropoxy)carbonyl)-L-leucinate (4) (3.0 g, 8.5 mmol) in THF (60 mL), and water (30 mL), was added LiOH.H₂O (1.08 g, 25.78 mmol). Reaction mixture was stirred at RT for 2 h. The progress of the reaction was monitored by TLC. After consumption of starting material, the excesses of THF was evaporated under reduced pressure. Then the mixture was acidified with 1N HCl, extracted with ethyl acetate (3×100 mL), and the combined organic layers were dried over sodium sulfate and evaporated under reduced pressure to afford ((1-(3-methoxybenzyl)cyclopropoxy)carbonyl)-L-leucine (5) which was used directly in the next step. TLC system: 10% Methanol in dichloromethane Rf: 0.1 LCMS (ESI): m/z 336.40 [M+Na]⁺

### Methyl (S)-2-((S)-2-(((1-(3-methoxybenzyl)cyclopropoxy)carbonyl)amino)-4-methylpentanamido)-3-((S)-2-oxopyrrolidin-3-yl)propanoate (6)

To a stirred solution of ((1-(3-methoxybenzyl)cyclopropoxy)carbonyl)-L-leucine (5) (2.2 g, 6.5 mmol) in DMF (40 mL) was added EDC.HCl (1.86 g, 9.7 mmol), HOBT (1.34 g, 9.7 mmol), DIPEA (3.39 mL, 19.5 mmol) was stirred at 0° C. for 10 minutes and then methyl (2S)-2-amino-3-(2-oxopyrrolidin-3-yl)propanoate (amine-fragment-2) (1.46 g, 7.8 mmol) was added at 0° C. simultaneously and stirred at room temperature for 16 h. Reaction mixture was diluted with ice water (100 mL), extracted with ethyl acetate (2×100 mL), dried over sodium sulfate and evaporated under reduced pressure. The crude residue was purified by silica gel column by eluting with 70% ethyl acetate/pet ether to afford methyl (S)-2-((S)-2-(((1-(3-methoxybenzyl)cyclopropoxy)carbonyl)amino)-4-methylpentanamido)-3-((S)-2-oxopyrrolidin-3-yl)propanoate (6). TLC system: 70% Ethyl acetate in Pet ether Rf: 0.5 LCMS (ESI): m/z 504.60 [M+H]⁺

### 1-(3-Methoxybenzyl)cyclopropyl ((S)-1-(((S)-1-hydroxy-3-((S)-2-oxopyrrolidin-3-yl)propan-2-yl)amino)-4-methyl-1-oxopentan-2-yl)carbamate (7)

To a stirred solution of methyl (S)-2-((S)-2-(((1-(3-methoxybenzyl)cyclopropoxy)carbonyl)amino)-4-methylpentanamido)-3-((S)-2-oxopyrrolidin-3-yl)propanoate (6) (0.530 g, 1.013 mmol) in DCM (20 mL), 2M LiBH₄ in THF (1.013 mL, 2.026 mmol) was added at 0° C. and stirred for 2 h. The progress of the reaction was monitored by TLC and LCMS. The reaction mixture was quenched with saturated NH₄Cl solution (10 mL) and extracted with DCM (2×50 mL), filtrate was concentrated under reduced pressure to afford 1-(3-Methoxybenzyl)cyclopropyl ((S)-1-(((S)-1-hydroxy-3-((S)-2-oxopyrrolidin-3-yl)propan-2-yl)amino)-4-methyl-1-oxopentan-2-yl)carbamate (7) TLC system: 5% Methanol in dichloromethane Rf: 0.4 LCMS (ESI): m/z 476.67 (M+H)⁺

### 1-(3-Methoxybenzyl)cyclopropyl ((S)-4-methyl-1-oxo-1-(((S)-1-oxo-3-((S)-2-oxopyrrolidin-3-yl)propan-2-yl)amino)pentan-2-yl)carbamate (Compound C42)

To a stirred solution of 1,2-diphenylethyl ((S)-1-(((S)-1-hydroxy-3-((S)-2-oxopyrrolidin-3-yl)propan-2-yl)amino)-4-methyl-1-oxopentan-2-yl)carbamate (7) (0.300 mg, 0.605 mmol) was dissolved in dichloromethane (20 mL) was added Dess-Martin periodinane (0.513 g, 1.216 mmol) at 0° C. and stirred at RT for 3 h. Reaction mixture was diluted with DCM (20 mL) and organic layer was washed with sat. Hypo solution (3×20 mL), sat. NaHCO₃ solution (3×20 mL). Organic layer was dried over anhydrous Na₂SO₄, filtered and concentrated and purified by Prep. HPLC to afford 1-(3-methoxybenzyl)cyclopropyl ((S)-4-methyl-1-oxo-1-(((S)-1-oxo-3-((S)-2-oxopyrrolidin-3-yl)propan-2-yl)amino)pentan-2-yl)carbamate (Compound C42). TLC system: 10% Methanol in dichloromethane Rf: 0.5 LCMS (ESI): m/z 474.62 (M+H)⁺

### (2S)-1-Hydroxy-2-((S)-2-(((1-(3-methoxybenzyl)cyclopropoxy)carbonyl)amino)-4-methylpentanamido)-3-((S)-2-oxopyrrolidin-3-yl)propane-1-sulfonate (Compound C49)

To a stirred solution of 1-(3-methoxybenzyl)cyclopropyl ((S)-4-methyl-1-oxo-1-(((S)-1-oxo-3-((S)-2-oxopyrrolidin-3-yl)propan-2-yl)amino)pentan-2-yl)carbamate (Compound C42) (0.100 mg, 0.2027 mmol) was dissolved in ethyl acetate (2.0 mL), ethanol (1.0 mL), water (0.4 mL), and then NaHSO₃ (0.0632 mg, 0.6081 mmol) was added and stirred at 40° C. for 4 h. The progress of the reaction was monitored by TLC and LCMS. The reaction mixture was filtered through celite pad and filtrate was dried over sodium sulphate, concentrated and the crude compound was washed with diethyl ether to afford ((2S)-1-hydroxy-2-((S)-2-(((1-(3-methoxybenzyl)cyclopropoxy)carbonyl)amino)-4-methylpentanamido)-3-((S)-2-oxopyrrolidin-3-yl)propane-1-sulfonate (Compound C49). TLC system: 10% Methanol in dichloromethane Rf: 0.2 LCMS (ESI): m/z 554.1 (M+H)⁺

### Example 41: Synthesis of Compound C43

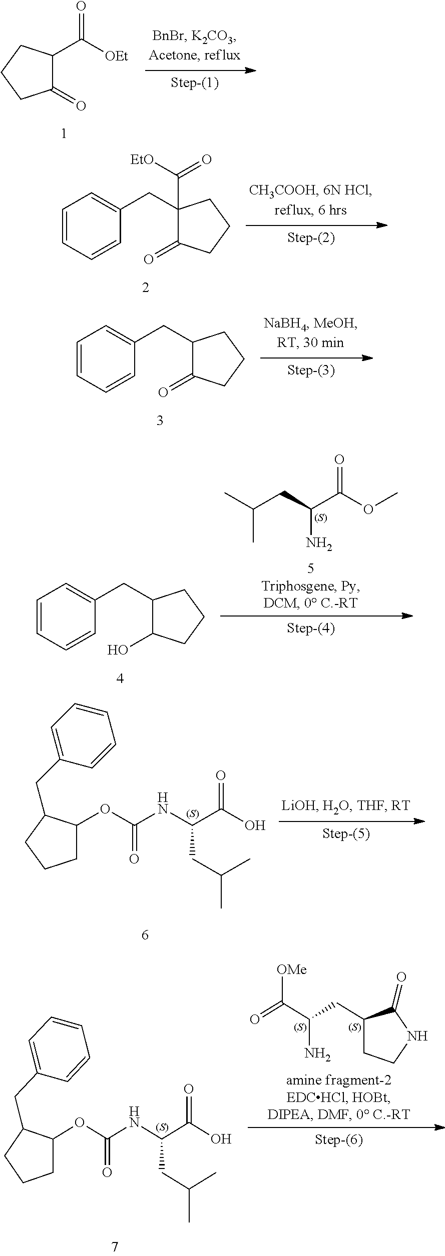

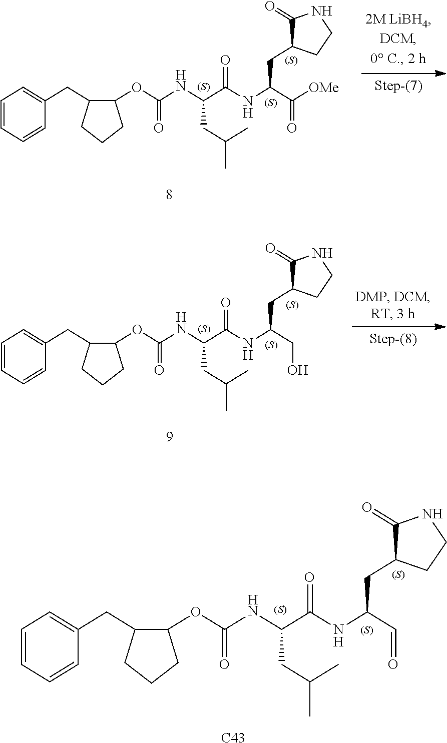

### Ethyl 1-benzyl-2-oxocyclopentane-1-carboxylate (2)

To a stirred solution of K₂CO₃ (17.6 g, 128.205 mmol) in Acetone (100 mL) was added ethyl 2-oxocyclopentane-1-carboxylate (1) (10 g, 64.102 mmol) at RT, followed by benzyl bromide (7.6 mL, 64.102 mmol) at RT and the reaction mixture was refluxed at 70° C. for 16 h. The progress of the reaction was monitored by TLC. Reaction mixture was cooled to RT and quenched with saturated NaHCO₃ (150 mL) then extracted with ethyl acetate (2×100 mL) then washed with the ethyl acetate layer with saturated NaHCO₃ (3×100 mL), dried over sodium sulfate and evaporated under reduced pressure. The crude residue was purified by silica gel column by eluting with 3% ethyl acetate in hexane to afford ethyl 1-benzyl-2-oxocyclopentane-1-carboxylate. TLC system: 10% Ethyl acetate in Hexane Rf: 0.5 LCMS (ESI): m/z=247.26 [M+H]⁺

### 2-benzylcyclopentan-1-one (3)

To a stirred solution of ethyl 1-benzyl-2-oxocyclopentane-1-carboxylate (2) (15 g, 60.975 mmol) in glacial acetic acid (150 mL) was added 6N aq.HCl (75 mL) at RT and the reaction mixture was refluxed at 70° C. for 6 h. The progress of the reaction was monitored by TLC. Reaction mixture was cooled to RT and poured into ice-cold water (100 mL) and extracted with ethyl acetate (3×50 mL), dried over sodium sulfate and evaporated under reduced pressure. The crude residue was purified by silica gel column by eluting with 2% ethyl acetate in hexane to afford 2-benzylcyclopentan-1-one (3). TLC system: 5% Ethyl acetate in Hexane Rf: 0.5 LCMS (ESI): m/z=175.21[M+H]+

### 2-benzylcyclopentan-1-ol (4)

To a stirred solution of 2-benzylcyclopentan-1-one (3) (5 g, 28.735 mmol) in MeOH (50 mL) was added NaBH₄ (1.06 g, 57.471 mmol) at 0° C. Reaction mixture was allowed to stir at RT for 30 min. The progress of the reaction was monitored by TLC. Reaction mixture was distilled and quenched with 1N HCl extracted with ethyl acetate (2×50 mL), dried over sodium sulfate and evaporated under reduced pressure to afford the crude compound 2-benzylcyclopentan-1-ol (4). TLC system: 5% Ethyl acetate in Hexane Rf: 0.2 LCMS (ESI): m/z=171.43 [M+H]⁺

### Methyl (((2-benzylcyclopentyl) oxy)carbonyl)-L-leucinate (6)

To a stirred solution of 2-benzylcyclopentan-1-ol (4) (5 vg, 28.409 mmol) in DCM (50 mL) was added pyridine (2.5 mL, 28.409 mmol), Triphosgene (2.1 g, 14.204 mmol) at 0° C. slowly, followed by methyl L-leucinate hydrochloride (5) (4.9 g, 34.091 mmol) and stirred at RT for 16 h. The progress of the reaction was monitored by TLC. Reaction mixture was quenched with ice water (50 mL), concentrated the organic layer, again washed with 1N aq HCl solution, then extracted with DCM (2×50 mL), dried over sodium sulfate and evaporated under reduced pressure. The crude residue was purified by silica gel column by eluting with 4% ethyl acetate in hexane to afford methyl (((2-benzylcyclopentyl)oxy)carbonyl)-L-leucinate (6). TLC system: 10% Ethyl acetate in Hexane Rf: 0.5 LCMS (ESI): m/z=348.33 [M+H]⁺

### (((2-benzylcyclopentyl) oxy) carbonyl)-L-leucine (7)

To a stirred solution of methyl (((2-benzylcyclopentyl) oxy) carbonyl)-L-leucinate (6) (4.5 g, 12.968 mmol) in THF (40 mL), DM water (20 mL), LiOH.H₂O (933 mg, 38.904 mmol) was added. Reaction mixture was stirred at RT for 2 h. The progress of the reaction was monitored by TLC. After consumption of starting material, the reaction mixture was concentrated and acidified with 1N aq. HCl, extracted with ethyl acetate (2×50 mL), dried over sodium sulfate and evaporated under reduced pressure to afford (((2-benzylcyclopentyl)oxy)carbonyl)-L-leucine (7). This crude was used for next step without any purification. TLC system: 5% Methanol in dichloromethane Rf: 0.2 LCMS (ESI): m/z=334.45 [M+H]⁺

### Methyl (2S)-2-((2S)-2-((((2-benzylcyclopentyl) oxy) carbonyl) amino)-4-methylpentanamido)-3-((S)-2-oxopyrrolidin-3-yl) propanoate (8)

To a stirred solution of (((2-benzylcyclopentyl)oxy)carbonyl)-L-leucine (7) (2 g, 6.006 mmol), in DMF (20 mL) was added EDC.HCl (1.72 g, 9.009 mmol), HOBT (1.21 g, 9.009 mmol), DIPEA (3.13 mL, 18.018 mmol) and methyl (2S)-2-amino-3-(2-oxopyrrolidin-3-yl)propanoate (amine-fragment-2) (1.34 g, 7.207 mmol) at 0° C. simultaneously and stirred at room temperature for 16 h. Reaction mixture was diluted with ice water (100 mL), extracted with ethyl acetate (2×100 mL), dried over sodium sulfate and evaporated under reduced pressure. The crude residue was purified by silica gel column by eluting with 5% methanol in dichloromethane to afford methyl (2S)-2-((2S)-2-((((2-benzylcyclopentyl)oxy)carbonyl)amino)-4-methylpentanamido)-3-((S)-2-oxopyrrolidin-3-yl)propanoate (8). TLC system: 5% Methanol in dichloromethane Rf: 0.3 LCMS (ESI): m/z=502.68 [M+H]⁺

### 2-benzylcyclopentyl ((S)-1-(((S)-1-hydroxy-3-((S)-2-oxopyrrolidin-3-yl) propan-2-yl) amino)-4-methyl-1-oxopentan-2-yl)carbamate (9)

To a stirred solution of methyl (2S)-2-((2S)-2-((((2-benzylcyclopentyl) oxy)carbonyl) amino)-4-methylpentanamido)-3-((S)-2-oxopyrrolidin-3-yl)propanoate (8) (1 g, 1.990 mmol) in Dichloromethane (10 mL), 2M LiBH₄ in THF (2 mL, 3.981 mmol) was added at 0° C. and stirred for 2 h The progress of the reaction was monitored by TLC and LCMS. the reaction mixture was quenched with saturated NH₄Cl solution and extracted with Dichloromethane (2×50 mL), filtrate was concentrated under reduced 2-benzylcyclopentyl ((S)-1-(((S)-1-hydroxy-3-((S)-2-oxo pyrrolidin-3-yl) propan-2-yl)amino)-4-methyl-1-oxopentan-2-yl)carbamate (9). TLC system: 5% Methanol in dichloromethane Rf: 0.4 LCMS (ESI): m/z=474.84 [M+H]⁺

### 2-benzylcyclopentyl ((S)-4-methyl-1-oxo-1-(((S)-1-oxo-3-((S)-2-oxopyrrolidin-3-yl) propan-2-yl) amino) pentan-2-yl) carbamate (Compound C43)

To a stirred solution of 2-benzylcyclopentyl ((S)-1-(((S)-1-hydroxy-3-((S)-2-oxo pyrrolidin-3-yl) propan-2-yl)amino)-4-methyl-1-oxopentan-2-yl) carbamate (9) (0.2 g, 0.422 mmol) was dissolved in dichloromethane (10 mL) was added Dess-Martin periodinane (0.537 g, 1.268 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude compound. The crude compound was purified by Prep. HPLC to afford 2-benzylcyclopentyl ((S)-4-methyl-1-oxo-1-(((S)-1-oxo-3-((S)-2-oxopyrrolidin-3-yl) propan-2-yl) amino) pentan-2-yl) carbamate (Compound C43). TLC system: 10% Methanol in dichloromethane Rf: 0.5 LCMS (ESI): m/z=472.62 (M+H)⁺

### Example 42: Synthesis of Compounds C44 and C48

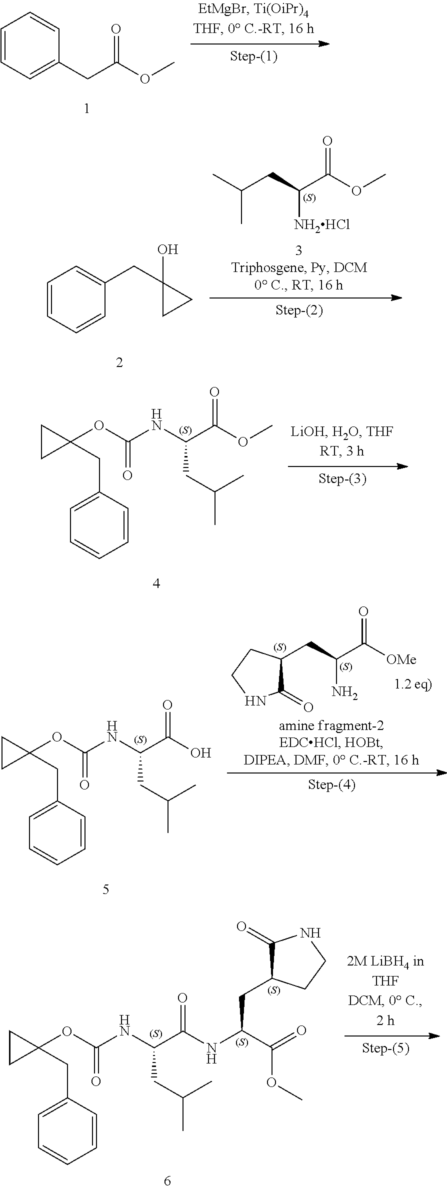

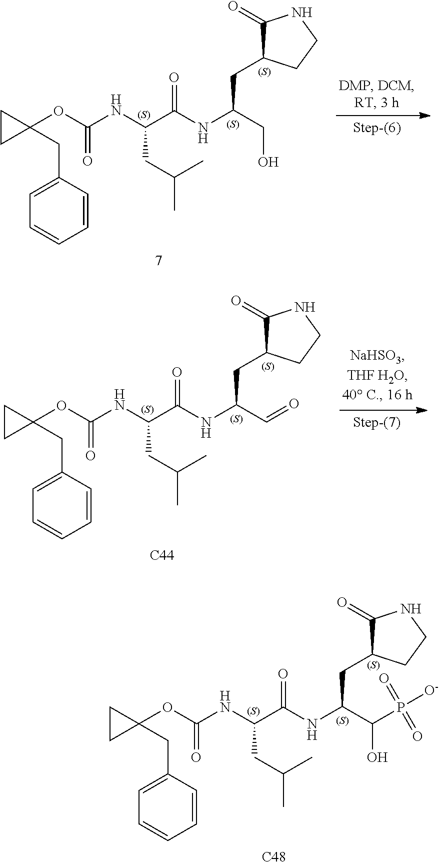

### 1-Benzylcyclopropan-1-ol (2)

To a stirred solution of methyl 2-phenylacetate (1) (5 g, 33.29 mmol) in THF (50 mL) was added titanium isoperoxide (13.2 mL, 46.61 mmol) at 0° C. slowly, followed by ethyl magnesium bromide (66 mL, 66.58 mmol) at 0° C. and the reaction mixture was stirred at RT for 16 h. The progress of the reaction was monitored by TLC. The reaction mixture was quenched with saturated NH₄Cl (150 mL) and filtered through celite pad and washed with ethyl acetate (100 mL), dried over sodium sulfate and evaporated under reduced pressure. The crude residue was purified by silica gel column by eluting with 7% ethyl acetate in hexane to afford 1-benzylcyclopropan-1-ol (2). TLC system: 20% Ethyl acetate in Hexane Rf: 0.2 LCMS (ESI): m/z=149.28 [M+H]^{+s}

### Methyl ((1-benzylcyclopropoxy) carbonyl)-L-leucinate (4)

To a stirred solution of 1-benzylcyclopropan-1-ol (2) (3.0 g, 20.25 mmol) in DCM (30 mL) was added pyridine (3 mL, 39.97 mmol), triphosgene (3 g, 10.12 mmol) at 0° C. slowly, followed by methyl L-leucinate hydrochloride (3) (3.5 g, 24.30 mmol) and stirred at RT for 16 h. The progress of the reaction was monitored by TLC. Reaction mixture was quenched with 1N aq. HCl (50 mL) then extracted with DCM (2×50 mL), the organic layer was dried over sodium sulfate and evaporated under reduced pressure. The crude residue was purified by silica gel column by eluting with 10% ethyl acetate in hexane to afford methyl ((1-benzylcyclopropoxy)carbonyl)-L-leucinate (4). TLC system: 20% Ethyl acetate in hexane Rf: 0.5 LCMS (ESI): m/z=320.52 [M+H]+

### ((1-Benzylcyclopropoxy) carbonyl)-L-leucine (5)

To a stirred solution of methyl ((1-benzylcyclopropoxy) carbonyl)-leucinate (4) (3.0, 9.40 mmol) in THF (15 mL), water (5 mL) was added LiOH.H₂O (677 mg, 28.21 mmol) at 0° C. Reaction mixture was stirred at RT for 3 h. The progress of the reaction was monitored by TLC. After consumption of starting material, the reaction mixture was concentrated and acidified with 1N HCl, extracted with ethyl acetate (2×30 mL), dried over sodium sulfate and evaporated under reduced pressure to afford ((1-benzylcyclopropoxy)carbonyl)-L-leucine (5) which was used directly in the next step. TLC system: 5% Methanol in dichloromethane Rf: 0.2 LCMS (ESI): m/z=306.50 [M+H]⁺

### Methyl (S)-2-((S)-2-(((1-benzylcyclopropoxy) carbonyl) amino)-4-methylpentanamido)-3-((S)-2-oxopyrrolidin-3-yl) propanoate (6)

To a stirred solution of ((1-benzylcyclopropoxy)carbonyl)-L-leucine (5) (2.5 g, 8.19 mmol) in DMF (20 mL) was added EDC.HCl (2.3 g, 12.29 mmol), HOBT (1.65 g, 12.29 mmol), DIPEA (4.2 mL, 24.58 mmol) and methyl (2S)-2-amino-3-(2-oxopyrrolidin-3-yl)propanoate (amine-fragment-2) (1.83 g, 9.83 mmol) at 0° C. simultaneously and stirred at room temperature for 16 h. Reaction mixture was diluted with ice water (50 mL), extracted with ethyl acetate (2×40 mL), the organic layer was dried over sodium sulfate and evaporated under reduced pressure to afford crude compound. The crude residue was purified by silica gel column by eluting with 5% methanol in dichloromethane to afford methyl (S)-2-((S)-2-(((1-benzylcyclopropoxy)carbonyl)amino)-4-methylpentanamido)-3-((S)-2-oxopyrrolidin-3-yl)propanoate (6). TLC system: 10% Methanol in dichloromethane Rf: 0.3 LCMS (ESI): m/z=508.63 [M+H]⁺

### 1-Benzylcyclopropyl ((S)-1-(((S)-1-hydroxy-3-((S)-2-oxopyrrolidin-3-yl) propan-2-yl) amino)-4-methyl-1-oxopentan-2-yl) carbamate (7)

To a stirred solution of methyl (S)-2-((S)-2-(((1-benzylcyclopropoxy)carbonyl)amino)-4-methylpentanamido)-3-((S)-2-oxopyrrolidin-3-yl)propanoate (6) (2.8 g, 5.91 mmol) in dichloromethane (30 mL) was added 2M LiBH₄ in THF (5.9 mL, 11.83 mmol) at 0° C. and stirred for 2 h. The progress of the reaction was monitored by TLC and LCMS. The reaction mixture was quenched with saturated NH₄Cl solution (50 mL) and extracted with DCM (2×50 mL), the organic layer was dried over sodium sulfate and evaporated under reduced pressure to afford 1-benzylcyclopropyl ((S)-1-(((S)-1-hydroxy-3-((S)-2-oxopyrrolidin-3-yl)propan-2-yl)amino)-4-methyl-1-oxopentan-2-yl)carbamate (7). TLC system: 10% Methanol in dichloromethane Rf: 0.2 LCMS (ESI): m/z=446.61 [M+H]⁺

### 1-Benzylcyclopropyl ((S)-4-methyl-1-oxo-1-(((S)-1-oxo-3-((S)-2-oxopyrrolidin-3-yl) propan-2-yl) amino) pentan-2-yl) carbamate (Compound C44)

To a stirred solution of 1-benzylcyclopropyl ((S)-1-(((S)-1-hydroxy-3-((S)-2-oxopyrrolidin-3-yl)propan-2-yl)amino)-4-methyl-1-oxopentan-2-yl)carbamate (7) (0.15 g, 0.33 mmol) was dissolved in dichloromethane (4 mL) was added Dess-Martin periodinane (226 mg, 1.01 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude product. The crude product was purified by Prep. HPLC to afford 1-benzylcyclopropyl ((S)-4-methyl-1-oxo-1-(((S)-1-oxo-3-((S)-2-oxopyrrolidin-3-yl) propan-2-yl) amino) pentan-2-yl) carbamate (Compound C44). TLC system: 10% Methanol in dichloromethane Rf: 0.5 LCMS (ESI): m/z=444.42 (M+H)⁺

### (2S)-2-((S)-2-(((1-Benzylcyclopropoxy) carbonyl) amino)-4-methylpentanamido)-1-hydroxy-3-((S)-2-oxopyrrolidin-3-yl) propane-1-sulfonate (Compound C48)

To a stirred solution of 1-benzylcyclopropyl ((S)-4-methyl-1-oxo-1-(((S)-1-oxo-3-((S)-2-oxopyrrolidin-3-yl)propan-2-yl)amino)pentan-2-yl)carbamate (50 mg, 0.11 mmol) in EtOAc (2 mL) was added EtOH/H₂O (2/1) (3 mL) and NaHSO₃ (35 mg, 0.33 mmol) at RT and the RM was stirred at 50° C. for 16 h. The progress of the reaction was monitored by TLC and LCMS. Reaction mixture was cooled to RT and filtered through celite pad and washed the celite pad with EtOH (10 mL) to afford crude compound. The crude was triturated with ethyl acetate and di ethyl ether to afford (2S)-2-((S)-2-(((1-benzylcyclopropoxy) carbonyl) amino)-4-methylpentanamido)-1-hydroxy-3-((S)-2-oxopyrrolidin-3-yl) propane-1-sulfonate (Compound C48). TLC system: 10% Methanol in dichloromethane Rf: 0.1 LCMS (ESI): m/z=526.2 (M+H)⁺ M=Free Base

### Example 43: Synthesis of Compounds C55 and C45

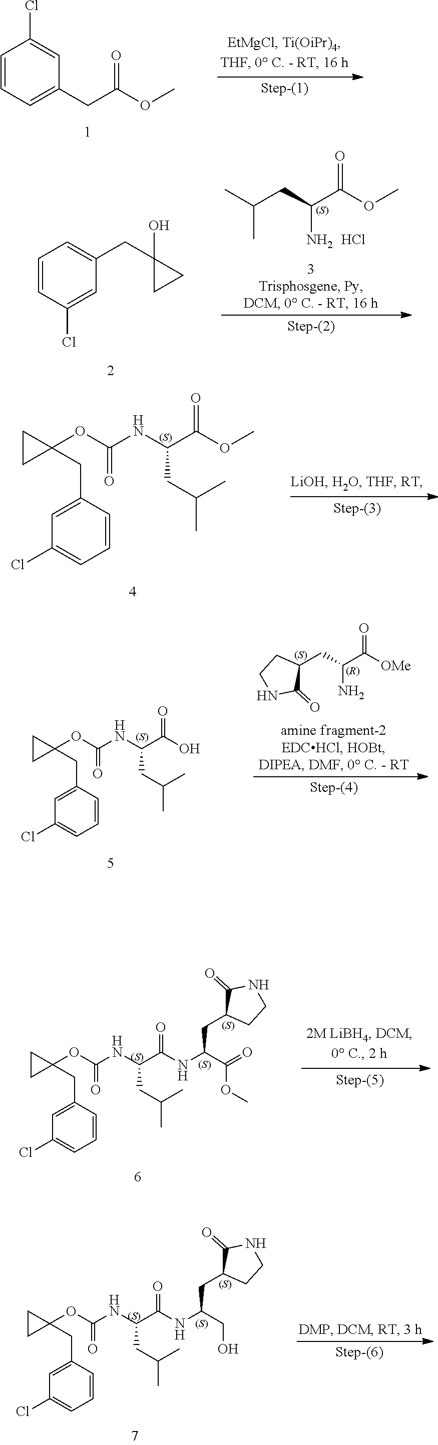

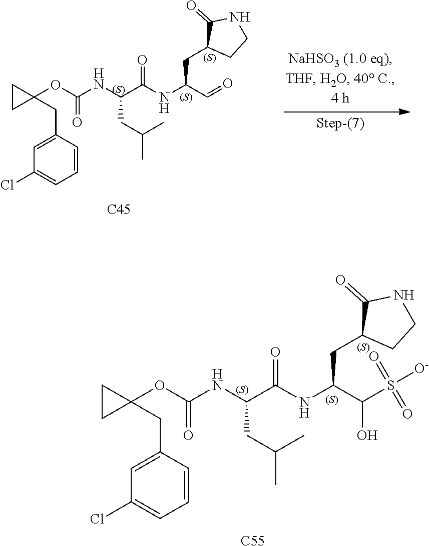

### 1-(3-Chlorobenzyl)cyclopropan-1-ol (2)

To a stirred solution of methyl 2-(3-chlorophenyl) acetate (1) (7 g, 38.043 mmol) in THF (50 mL) was added Titanium isoperoxide (15 mL, 53.260 mmol) at 0° C. slowly, followed by ethyl magnesium bromide (95 mL, 95.108 mmol) at 0° C. and the reaction mixture was stirred at RT for 16 h. The progress of the reaction was monitored by TLC. The reaction mixture was quenched with saturated NH₄Cl (150 mL) and filtered through celite pad and washed with ethyl acetate (200 mL), dried over sodium sulfate and evaporated under reduced pressure. The crude residue was purified by silica gel column by eluting with 5% ethyl acetate in hexane to afford 1-(3-chloro benzyl) cyclopropan-1-ol (2) (4.2 g, 23.076 mmol, 60% yield) as off-white solid. TLC system: 10% Ethyl acetate in Hexane Rf: 0.2 LCMS (ESI): m/z=183.33 [M+H]⁺

### Methyl ((1-(3-chlorobenzyl)cyclopropoxy)carbonyl)-L-leucinate (4)

To a stirred solution of 1-(3-chlorobenzyl)cyclopropan-1-ol (2) (3.7 g, 20.329 mmol) in DCM (50 mL) was added pyridine (1.5 mL, 16.8 mmol), Triphosgene (3 g, 10.164 mmol) at 0° C. slowly, followed by methyl L-leucinate hydrochloride (3) (3.4 g, 24.395 mmol) and stirred at RT for 16 h. The progress of the reaction was monitored by TLC. Reaction mixture was quenched with ice water (50 mL), concentrated the organic layer, again washed with 1N aq. HCl, then extracted with DCM (2×50 mL), the organic layer was dried over sodium sulfate and evaporated under reduced pressure. The crude residue was purified by silica gel column by eluting with 10% ethyl acetate in hexane to afford methyl ((1-(3-chlorobenzyl)cyclopropoxy)carbonyl)-L-leucinate (4) (3.2 g, 9.065 mmol, 44% yield) as yellow oily liquid. TLC system: 20% Ethyl acetate in Hexane Rf: 0.5 LCMS (ESI): m/z=354.26 [M+H]+

### ((1-3-Chlorobenzyl)cyclopropoxy)carbonyl)-L-leucine (5)

To a stirred solution of methyl ((1-(3-chlorobenzyl)cyclopropoxy)carbonyl)-L-leucinate (4) (3.2 g, 9.065 mmol) in THF (40 mL), water (20 mL), LiOH.H₂O (652 mg, 27.195 mmol) was added. Reaction mixture was stirred at RT for 2 h. The progress of the reaction was monitored by TLC. After consumption of starting material, the reaction mixture was concentrated and acidified with 1N HCl, extracted with ethyl acetate (2×50 mL), dried over sodium sulfate and evaporated under reduced pressure to afford ((1-(3-chlorobenzyl)cyclopropoxy)carbonyl)-L-leucine (5) (2.8 g crude, 8.259 mmol, 91% yield) as yellow gummy liquid. This crude was used for next step without any purification. TLC system: 5% Methanol in dichloromethane Rf: 0.5 LCMS (ESI): m/z=340.23 [M+H]⁺

### Methyl (S)-2-((S)-2-(((1-(3-chlorobenzyl)cyclopropoxy)carbonyl)amino)-4-methylpentanamido)-3-((S)-2-oxopyrrolidin-3-yl)propanoate (6)

To a stirred solution of ((1-(3-chlorobenzyl)cyclopropoxy)carbonyl)-L-leucine (5) (2.8 g, 8.2595 mmol) (5) in DMF (50 mL) was added EDC.HCl (2.36 g, 12.389 mmol), HOBT (1.67 g, 12.389 mmol), DIPEA (4.45 mL, 24.778 mmol) and methyl (2S)-2-amino-3-(2-oxopyrrolidin-3-yl)propanoate (amine-fragment-2) (1.68 g, 9.085 mmol) at 0° C. simultaneously and stirred at room temperature for 16 h. Reaction mixture was diluted with ice water (100 mL), extracted with ethyl acetate (2×100 mL), the organic layer was dried over sodium sulfate and evaporated under reduced pressure to afford crude compound. The crude residue was purified by silica gel column by eluting with 5% methanol in dichloromethane to afford methyl (S)-2-((S)-2-(((1-(3-chlorobenzyl) cyclopropoxy) carbonyl) amino)-4-methylpentanamido)-3-((S)-2-oxopyrrolidin-3-yl)propanoate (6) (2.3 g, 4.536 mmol, 54% yield) as yellow gummy liquid. TLC system: 10% Methanol in dichloromethane Rf: 0.3 LCMS (ESI): m/z=508.63 [M+H]⁺

### 1-(3-Chlorobenzyl)cyclopropyl ((S)-1-(((S)-1-hydroxy-3-((S)-2-oxopyrrolidin-3-yl)propan-2-yl)amino)-4-methyl-1-oxopentan-2-yl)carbamate (7)

To a stirred solution of methyl (S)-2-((S)-2-(((1-(3-chlorobenzyl) cyclopropoxy) carbonyl) amino)-4-methylpentanamido)-3-((S)-2-oxopyrrolidin-3-yl)propanoate (6) (2.2 g, 4.339 mmol) in Dichloromethane (20 mL), 2M LiBH₄ in THF (4.3 mL, 8.678 mmol) was added at 0° C. and stirred for 2 h. The progress of the reaction was monitored by TLC and LCMS. The reaction mixture was quenched with saturated NH₄Cl solution (50 mL) and extracted with DCM (2×50 mL), the organic layer was dried over sodium sulfate and evaporated under reduced pressure to afford. 1-(3-chlorobenzyl)cyclopropyl ((S)-1-(((S)-1-hydroxy-3-((S)-2-oxopyrrolidin-3-yl)propan-2-yl)amino)-4-methyl-1-oxopentan-2-yl)carbamate (7) (1.3 g, 2.713 mmol, 62% yield) as an off-white solid. TLC system: 10% Methanol in dichloromethane Rf: 0.4 LCMS (ESI): m/z=480.00 [M+H]⁺

### 1-(3-Chlorobenzyl) cyclopropyl ((S)-4-methyl-1-oxo-1-(((S)-1-oxo-3-((S)-2-oxopyrrolidin-3-yl)propan-2-yl)amino)pentan-2-yl)carbamate (Compound C45)

To a stirred solution of 1-(3-chlorobenzyl)cyclopropyl ((S)-1-(((S)-1-hydroxy-3-((S)-2-oxopyrrolidin-3-yl)propan-2-yl)amino)-4-methyl-1-oxopentan-2-yl) carbamate (7) (0.18 g, 0.375 mmol) was dissolved in dichloromethane (10 mL) was added Dess-Martin periodinane (0.477 g, 1.127 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude product. The crude product was purified by Prep. HPLC to afford 1-(3-chlorobenzyl) cyclopropyl ((S)-4-methyl-1-oxo-1-(((S)-1-oxo-3-((S)-2-oxopyrrolidin-3-yl) propan-2-yl) amino)pentan-2-yl) carbamate (Compound C45) (33 mg, 0.069 mmol, 18% yield) as an off-white solid. TLC system: 10% Methanol in dichloromethane Rf: 0.5 LCMS (ESI): m/z=478.41 (M+H)⁺

### ((2S)-2-((S)-2-(((1-(3-chlorobenzyl)cyclopropoxy)carbonyl)amino)-4-methylpentanamido)-1-hydroxy-3-((S)-2-oxopyrrolidin-3-yl)propane-1-sulfonate (Compound C55)

To a stirred solution of 1-(3-chlorobenzyl) cyclopropyl ((S)-4-methyl-1-oxo-1-(((S)-1-oxo-3-((S)-2-oxopyrrolidin-3-yl) propan-2-yl) amino)pentan-2-yl) carbamate (Compound C45) (0.100 mg, 0.2027 mmol) was dissolved in ethyl acetate (2.0 mL), ethanol (1.0 mL), water (0.4 mL), and then NaHSO₃ (0.0632 mg, 0.6081 mmol) was added and stirred at 40° C. for 4 h. The progress of the reaction was monitored by TLC and LCMS. The reaction mixture was filtered through celite pad and filtrate was dried over sodium sulphate, concentrated and the crude compound was washed with diethyl ether to afford (2S)-2-((S)-2-(((1-(3-chlorobenzyl)cyclopropoxy)carbonyl)amino)-4-methylpentanamido)-1-hydroxy-3-((S)-2-oxopyrrolidin-3-yl)propane-1-sulfonate (Compound C55) (25 mg, 0.047 mmol, 26% yield) as an off-white solid. TLC system: 10% Methanol in dichloromethane Rf: 0.2 LCMS (ESI): m/z 558.1 (M+H)⁺

### Example 44: Synthesis of Compounds C46 and C57

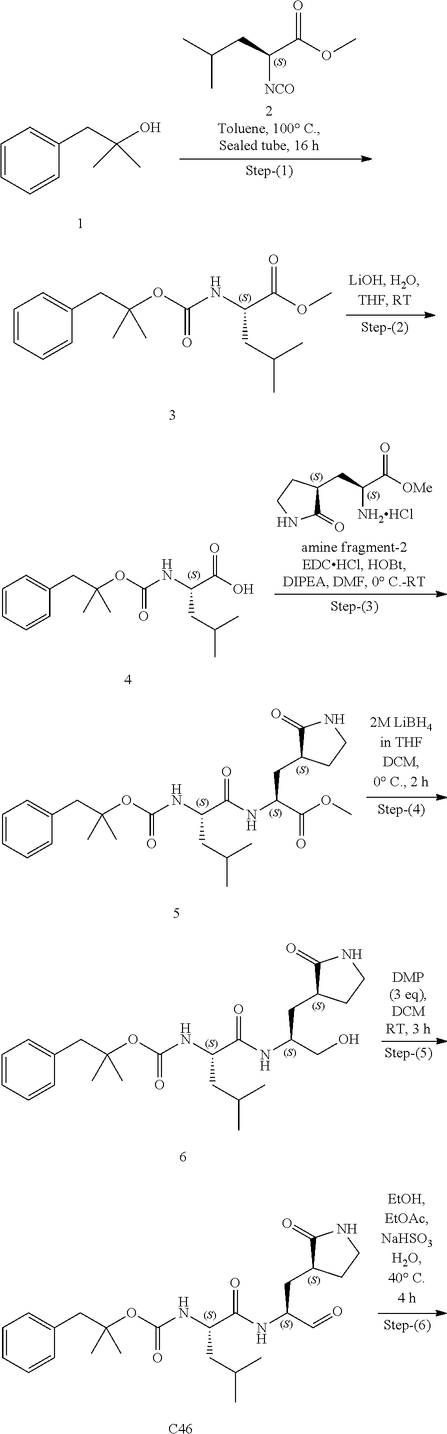

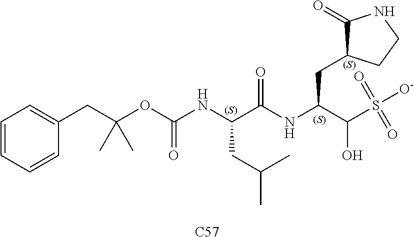

### Methyl (((2-methyl-1-phenylpropan-2-yl) oxy) carbonyl)-L-leucinate (3)

To a stirred solution of 2-methyl-1-phenylpropan-2-ol (1) (2 g, 13.31 mmol) in toluene (20 mL) was added methyl (S)-2-isocyanato-4-methylpentanoate (2.7 g, 15.98 mmol) at RT and heated to 100° C. under sealed tube for 16 h. The progress of the reaction was monitored by TLC. Reaction mixture was evaporated under reduced pressure to afford crude residue, crude residue was purified by normal phase chromatography to afford methyl (((2-methyl-1-phenylpropan-2-yl) oxy) carbonyl)-L-leucinate (3). TLC system: 20% Ethyl acetate in hexane Rf: 0.5 LCMS (ESI): m/z 344.41 [M+Na]⁺

### (((2-Methyl-1-phenylpropan-2-yl) oxy) carbonyl)-L-leucine (4)

To a stirred solution of methyl (((2-methyl-1-phenylpropan-2-yl)oxy)carbonyl)-L-leucinate (3) (3.2 g, 9.96 mmol) in THF (30 mL), water (10 mL) was added lithium hydroxide (717 mg, 29.90 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford (((2-methyl-1-phenylpropan-2-yl)oxy)carbonyl)-L-leucine (4). TLC system: 100% EtOAc Rf: 0.1 LCMS (ESI): m/z 330.50 [M+Na]⁺

### Methyl (S)-2-((S)-4-methyl-2-((((2-methyl-1-phenylpropan-2-yl) oxy) carbonyl) amino) pentanamido)-3-((S)-2-oxopyrrolidin-3-yl) propanoate (5)

To a stirred solution of (((2-methyl-1-phenylpropan-2-yl)oxy)carbonyl)-L-leucine (2 g, 6.51 mmol) DMF (15 mL) added EDC.HCl (1.86 g, 9.77 mmol), HOBt (1.31 g, 9.77 mmol), DIPEA (3.6 mL, 19.54 mmol) and methyl (S)-2-amino-3-((S)-2-oxopyrrolidin-3-yl)propanoate hydrochloride (amine fragment-2) (1.4 g, 7.81 mmol) at 0° C. simultaneously and stirred at room temperature for 16 h. Reaction mixture was diluted with ice water (50 mL), extracted with ethyl acetate (2×40 mL), dried over sodium sulfate and evaporated under reduced pressure. The crude residue was purified by combi-flash NP, compound eluted at 3% methanol in dichloromethane to afford methyl (S)-2-((S)-4-methyl-2-((((2-methyl-1-phenylpropan-2-yl)oxy)carbonyl)amino)pentanamido)-3-((S)-2-oxopyrrolidin-3-yl)propanoate (5). TLC system: 10% Methanol in dichloromethane Rf: 0.4 LCMS (ESI): m/z 476.37 [M+H]⁺

### 2-Methyl-1-phenylpropan-2-yl((S)-1-(((S)-1-hydroxy-3-((S)-2-oxopyrrolidin-3-yl) propan-2-yl) amino)-4-methyl-1-oxopentan-2-yl) carbamate (6)

To a stirred solution of methyl (S)-2-((S)-4-methyl-2-((((2-methyl-1-phenylpropan-2-yl)oxy)carbonyl)amino)pentanamido)-3-((S)-2-oxopyrrolidin-3-yl)propanoate (5) (2 g, 4.21 mmol) in DCM (20 mL) was added 2M LiBH₄ in THF (4.2 mL, 8.42 mmol) at 0° C. and the reaction mixture stirred for 2 h at 0° C. The progress of the reaction was monitored by TLC and LCMS. Reaction mixture was quenched with sat. ammonium chloride solution (40 mL) and extracted with DCM (2×30 mL). Organic layer was washed with brine solution (30 mL), dried over Na₂SO₄ and concentrated to afford 2-methyl-1-phenylpropan-2-yl ((S)-1-(((S)-1-hydroxy-3-((S)-2-oxopyrrolidin-3-yl)propan-2-yl)amino)-4-methyl-1-oxopentan-2-yl)carbamate (6). TLC system: 10% Methanol in dichloromethane Rf: 0.2 LCMS (ESI): m/z 448.49 [M+H]⁺

### 2-Methyl-1-phenylpropan-2-yl ((S)-4-methyl-1-oxo-1-(((S)-1-oxo-3-((S)-2-oxopyrrolidin-3-yl) propan-2-yl) amino) pentan-2-yl) carbamate (Compound C46)

To a stirred solution of 2-methyl-1-phenylpropan-2-yl ((S)-1-(((S)-1-hydroxy-3-((S)-2-oxopyrrolidin-3-yl)propan-2-yl)amino)-4-methyl-1-oxopentan-2-yl)carbamate (6) (200 mg, 0.89 mmol) was dissolved in dichloromethane (5 mL) was added Dess-Martin periodinane (599 mg, 2.67 mmol) at 0° C. and stirred at RT for 3 h. Reaction mixture was diluted with DCM (15 mL) followed by sat. Hypo solution (3×15 mL), followed by sat. NaHCO₃ solution (3×15 mL). Organic layer was dried over anhydrous Na₂SO₄, filtered and concentrated to get crude compound. The crude compound was purified by prep HPLC to afford 2-methyl-1-phenylpropan-2-yl ((S)-4-methyl-1-oxo-1-(((S)-1-oxo-3-((S)-2-oxopyrrolidin-3-yl)propan-2-yl)amino)pentan-2-yl)carbamate (Compound C46). TLC system: 10% Methanol in dichloromethane Rf: 0.5 LCMS (ESI): m/z 446.45 (M+H)⁺

### (2S)-1-hydroxy-2-((S)-4-methyl-2-((((2-methyl-1-phenylpropan-2-yl)oxy)carbonyl)amino)pentanamido)-3-((S)-2-oxopyrrolidin-3-yl)propane-1-sulfonate (Compound C57)

To a stirred solution of 2-methyl-1-phenylpropan-2-yl ((S)-4-methyl-1-oxo-1-(((S)-1-oxo-3-((S)-2-oxopyrrolidin-3-yl)propan-2-yl)amino)pentan-2-yl)carbamate (Compound C46) (0.100 g, 0.231 mmol) was dissolved in Ethyl acetate (2.0 mL), Ethanol (1.0 mL), Water (0.4 mL), and then NaHSO₃ (0.0361 mg, 0.347 mmol) was added and stirred at 40° C. for 16 h. The progress of the reaction was monitored by TLC and LCMS. The reaction mixture was filtered through celite pad and filtrate was dried over sodium sulphate, concentrated and the crude compound was washed with diethyl ether to afford (2S)-1-hydroxy-2-((S)-4-methyl-2-((((2-methyl-1-phenylpropan-2-yl)oxy)carbonyl)amino)pentanamido)-3-((S)-2-oxopyrrolidin-3-yl)propane-1-sulfonate (Compound C57). TLC system: 15% Methanol in dichloromethane Rf: 0.2 LCMS (ESI): m/z 526.12 (M+H)⁺

### Example 45: Synthesis of Compound C47

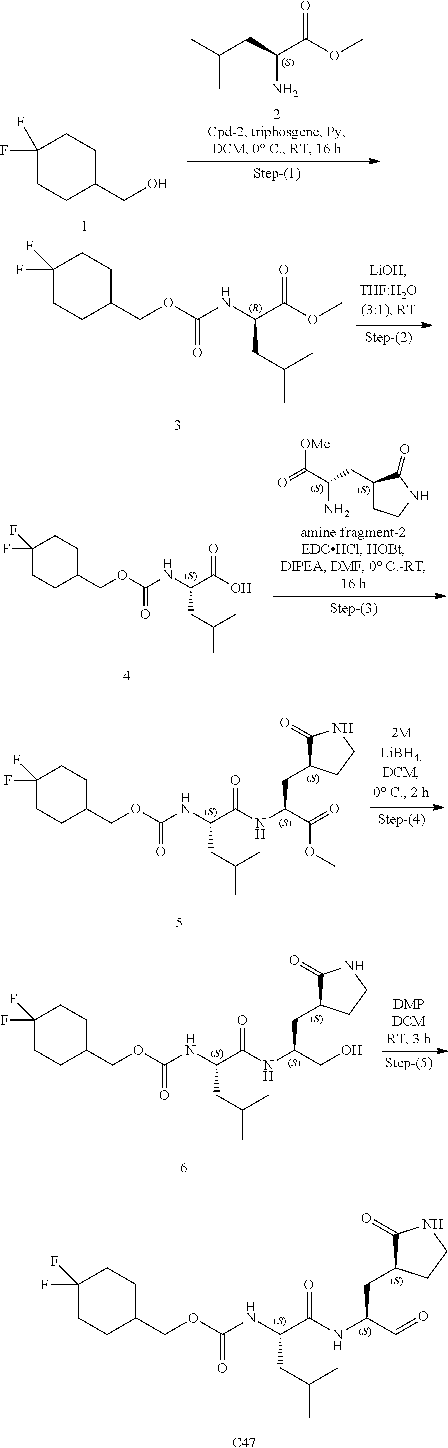

### Methyl (((4,4-difluorocyclohexyl)methoxy)carbonyl)-D-leucinate (3)

To a stirred solution of (4,4-difluorocyclohexyl)methanol (1) (9.5 g, 63.33 mmol) in DCM (100 mL) was added pyridine (10 mL, 63.33 mmol), Triphosgene (9.32 g, 31.66 mmol) at 0° C. slowly, followed by methyl L-leucinate hydrochloride (2) (11.46 g, 63.33 mmol) and stirred at RT for 16 h. The progress of the reaction was monitored by TLC. Reaction mixture was quenched with ice water (150 mL), concentrated the organic layer, again washed with 1N HCL solution, then extracted with DCM (2×150 mL), dried over sodium sulfate and evaporated under reduced pressure. The crude residue was purified by silica gel column by eluting with 20% ethyl acetate in hexane to afford methyl (((4,4-difluorocyclohexyl)methoxy)carbonyl)-D-leucinate (3). TLC system: 20% Ethyl acetate in Pet ether Rf: 0.8 LCMS (ESI): m/z 322.1[M+H]+

### (((4,4-Difluorocyclohexyl)methoxy)carbonyl)-L-leucine (4)

To a stirred solution of methyl (((4,4-difluorocyclohexyl)methoxy)carbonyl)-D-leucinate (3) (13 g, 40.49 mmol) in THF (130 mL), and water (75 mL), LiOH.H₂O (5 g, 121.49 mmol) was added. Reaction mixture was stirred at RT for 2 h. The progress of the reaction was monitored by TLC. After consumption of starting material, the reaction mixture was concentrated and acidified with 1N HCl, extracted with ethyl acetate (4×100 mL), the combined organic layer was dried over sodium sulfate and evaporated under reduced pressure to afford (((4,4-difluorocyclohexyl)methoxy)carbonyl)-L-leucine (4) which was used directly in the next step. TLC system: 10% Methanol in dichloromethane Rf: 0.1 LCMS (ESI): m/z 308.1 [M+H]⁺

### Methyl (S)-2-((S)-2-((((4,4-difluorocyclohexyl)methoxy)carbonyl)amino)-4-methylpentanamido)-3-((S)-2-oxopyrrolidin-3-yl)propanoate (5)

To a stirred solution of (((4,4-difluorocyclohexyl)methoxy)carbonyl)-L-leucine (4) (5 g, 16.28 mmol) in DMF (100 mL) was added EDC.HCl (4.58 g, 24.42 mmol), HOBT (3.33 g, 24.42 mmol), DIPEA (8.36 mL, 48.85 mmol) was stirred at 0° C. for 10 minutes and then methyl (2S)-2-amino-3-(2-oxopyrrolidin-3-yl)propanoate (amine-fragment-2) (3.65 g, 19.54 mmol) was added at 0° C. simultaneously and stirred at room temperature for 16 h. Reaction mixture was diluted with ice water (200 mL), extracted with ethyl acetate (3×100 mL), dried over sodium sulfate and evaporated under reduced pressure. The crude residue was purified by silica gel column by eluting with 70% ethyl acetate/pet ether to afford methyl (S)-2-((S)-2-((((4,4-difluorocyclohexyl)methoxy)carbonyl)amino)-4-methylpentanamido)-3-((S)-2-oxopyrrolidin-3-yl)propanoate (5). TLC system: 70% Ethyl acetate in Pet ether Rf: 0.5 LCMS (ESI): m/z 476.54 [M+H]⁺

### (4,4-Difluorocyclohexyl)methyl ((S)-1-(((S)-1-hydroxy-3-((S)-2-oxopyrrolidin-3-yl)propan-2-yl)amino)-4-methyl-1-oxopentan-2-yl)carbamate (6)

To a stirred solution of methyl (S)-2-((S)-2-((((4,4-difluorocyclohexyl)methoxy)carbonyl)amino)-4-methylpentanamido)-3-((S)-2-oxopyrrolidin-3-yl)propanoate (5) (2.8 g, 5.894 mmol) in DCM (20 mL), 2M LiBH₄ in THF (5.89 mL, 11.789 mmol) was added at 0° C. and stirred for 2 h. The progress of the reaction was monitored by TLC and LCMS. The reaction mixture was quenched with saturated NH₄Cl solution (50 mL) and extracted with DCM (3×50 mL), filtrate was concentrated under reduced pressure to afford 1-(3-Methoxybenzyl)cyclopropyl (4,4-difluorocyclohexyl)methyl ((S)-1-(((S)-1-hydroxy-3-((S)-2-oxopyrrolidin-3-yl)propan-2-yl)amino)-4-methyl-1-oxopentan-2-yl)carbamate (6) TLC system: 5% Methanol in dichloromethane Rf: 0.4 LCMS (ESI): m/z 448.63 (M+H)⁺

### (4,4-Difluorocyclohexyl)methyl ((S)-4-methyl-1-oxo-1-(((S)-1-oxo-3-((S)-2-oxopyrrolidin-3-yl)propan-2-yl)amino)pentan-2-yl)carbamate (Compound C47)

To a stirred solution of (4,4-difluorocyclohexyl)methyl ((S)-1-(((S)-1-hydroxy-3-((S)-2-oxopyrrolidin-3-yl)propan-2-yl)amino)-4-methyl-1-oxopentan-2-yl)carbamate (7) (250 mg, 0.526 mmol) was dissolved in ethyl acetate (20 mL) was added Dess-Martin periodinane (669 mg, 1.57 mmol) at 0° C. for 10 min and stirred at RT for 3 h. Reaction mixture was diluted with ethyl acetate (20 mL) and organic layer was washed with sat. Hypo solution (3×20 mL), sat. NaHCO₃ solution (3×20 mL). Organic layer was dried over anhydrous Na₂SO₄, filtered and concentrated and purified by Prep. HPLC to afford (4,4-difluorocyclohexyl)methyl ((S)-4-methyl-1-oxo-1-(((S)-1-oxo-3-((S)-2-oxopyrrolidin-3-yl)propan-2-yl)amino)pentan-2-yl)carbamate (Compound C47). TLC system: 10% Methanol in dichloromethane Rf: 0.5 LCMS (ESI): m/z 446.55 (M+H)⁺

### Example 46: Synthesis of Compound C33

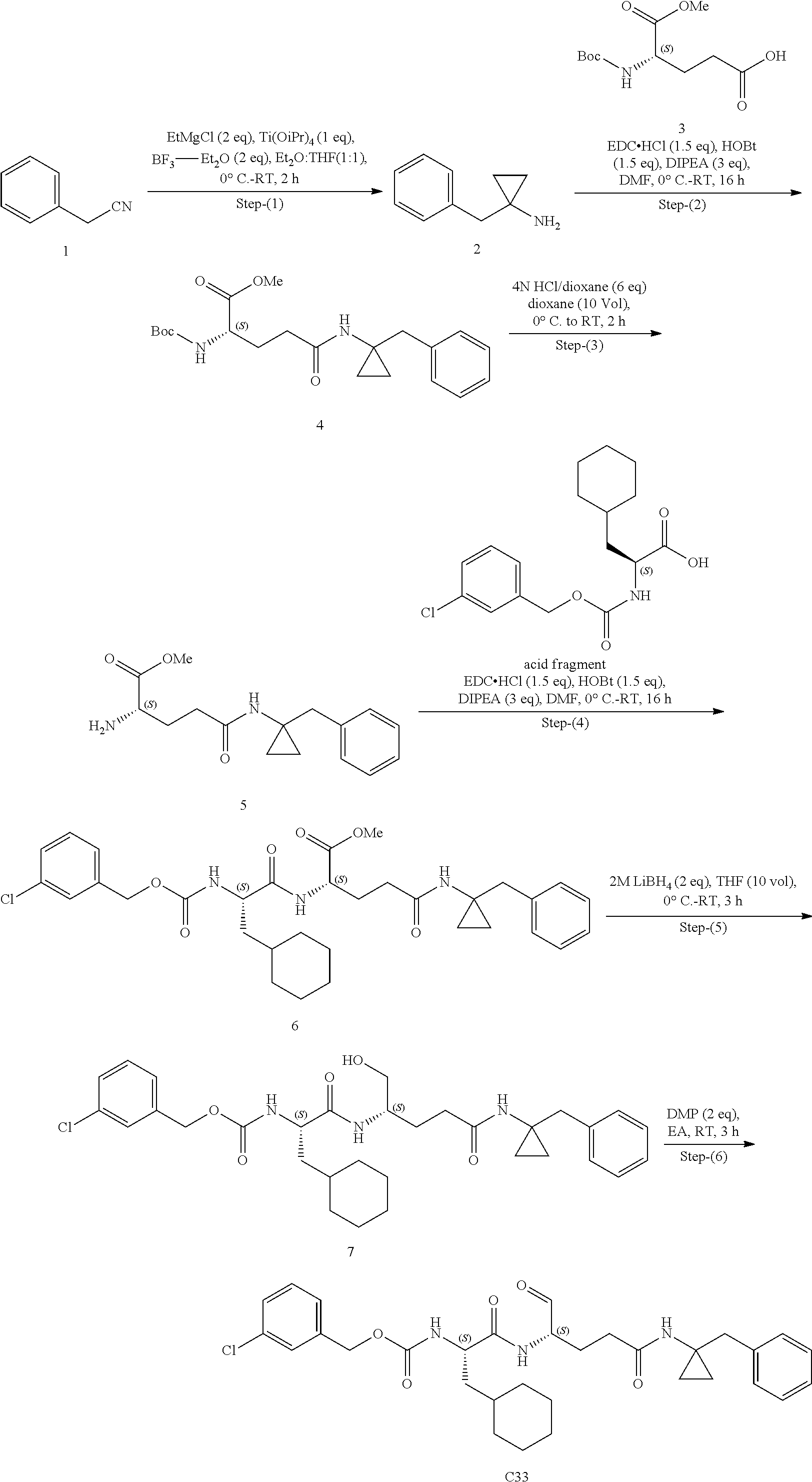

### 1-benzylcyclopropan-1-amine (2)

To a stirred solution of 2-phenylacetonitrile (1) (2 g, 17.094 mmol) in Et₂O:THF (1:1) (20 mL) were added Titanium isopropoxide (5.14 g, 18.119 mmol) and followed by added 2M Ethyl magnesium chloride in THF (17 mL, 34.188 mmol) slowly drop wise for 10 min at 0° C. Then the reaction mixture stirred at RT for 1 h and then added BF₃-Et₂O (4.8 mL, 34.188 mmol) slowly at 0° C. for 15 min (exothermic occurred) and stirred at room temperature for 1 h. The progress of the reaction was monitored by TLC and LCMS. After 1 h, the reaction mixture was poured in 10% NaOH solution (100 mL) white precipitate formed. The reaction mixture filtered through celite bed and washed with ethyl acetate (2×50 mL) and filtrate washed with brine solution (100 ml) and combined organic layer was dried over sodium sulfate, filtered and evaporated under reduced pressure. The crude residue was purified by combi-flash compound eluted at 70% Ethyl acetate in pet ether to afford 1-benzylcyclopropan-1-amine (2). TLC system: 80% Ethyl acetate in Pet ether Rf: 0.3 LCMS (ESI): m/z 148.11 [M+H]⁺

### Methyl N5-(1-benzylcyclopropyl)-N2-(tert-butoxycarbonyl)-L-glutaminate (4)

To a stirred solution of (S)-4-((tert-butoxycarbonyl)amino)-5-methoxy-5-oxopentanoic acid (3) (1 g, 1.915 mmol) in DMF (10 mL) were added EDC.HCl (731 mg, 3.831 mmol), HOBT (517 mg, 3.831 mmol), DIPEA (1 mL, 5.747 mmol) and 1-benzylcyclopropan-1-amine (2) (337 mg, 2.298 mmol) at 0° C. simultaneously and stirred at room temperature for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 40% Ethyl acetate in pet ether to afford methyl N5-(1-benzylcyclopropyl)-N2-(tert-butoxycarbonyl)-L-glutaminate (4). TLC system: 50% Ethyl acetate in Pet ether Rf: 0.5 LCMS (ESI): m/z=413 [M+Na]⁺

### Methyl N5-(1-benzylcyclopropyl)-L-glutaminate hydrochloride (5)

To a stirred solution of methyl N5-(1-benzylcyclopropyl)-N2-(tert-butoxycarbonyl)-L-glutaminate (4) (1.0 g, 2.564 mmol) in 1,4-dioxane (10 mL) was added 4N HCl in dioxane (20 mL) with drop wise at 0° C. and the reaction mixture was stirred at RT for 2 h. The progress of the reaction was monitored by TLC and LCMS. After consumption of starting material, the reaction mixture was evaporated under reduced pressure to obtained crude compound, the resulting crude triturated with diethyl ether to afford methyl N5-(1-benzylcyclopropyl)-L-glutaminate hydrochloride (5). TLC system: 10% Methanol in DCM Rf: 0.2 LCMS (ESI): m/z 291.28 [M+H]⁺

### Methyl N5-(1-benzylcyclopropyl)-N2-((S)-2-((((3-chlorobenzyl)oxy)carbonyl)amino)-3-cyclohexylpropanoyl)-L-glutaminate (6)

To a stirred solution of (S)-2-((((3-chlorobenzyl)oxy)carbonyl)amino)-3-cyclohexylpropanoic acid (acid fragment) (1.0 g, 2.949 mmol) DMF (10 mL) added EDC.HCl (1.12 g, 5.899 mmol), HOBT (796 mg, 5.899 mmol), DIPEA (1.6 mL, 8.849 mmol) and methyl N5-(1-benzylcyclopropyl)-L-glutaminate hydrochloride (5) (855 mg, 3.539 mmol) at 0° C. simultaneously and stirred at room temperature for 16 h. The progress of the reaction was monitored by TLC and LCMS. After 16 h, reaction mixture was diluted with ice water (30 mL), extracted with ethyl acetate (2×50 mL). The combined organic layer was dried over sodium sulfate, filtered and evaporated under reduced pressure. The crude residue was purified by combi-flash, compound eluted at 60% Ethyl acetate in pet ether to afford methyl N5-(1-benzylcyclopropyl)-N2-((S)-2-((((3-chlorobenzyl)oxy)carbonyl)amino)-3-cyclohexylpropanoyl)-L-glutaminate (6). TLC system: 80% Ethyl acetate in Pet ether Rf: 0.5 LCMS (ESI): m/z 612.88 [M+H]⁺

### 3-Chlorobenzyl ((S)-1-(((S)-5-((1-benzylcyclopropyl)amino)-1-hydroxy-5-oxopentan-2-yl)amino)-3-cyclohexyl-1-oxopropan-2-yl)carbamate (7)

To a stirred solution of methyl N5-(1-benzylcyclopropyl)-N2-((S)-2-((((3-chlorobenzyl)oxy)carbonyl)amino)-3-cyclohexylpropanoyl)-L-glutaminate (6) (700 mg, 1.143 mmol) in THF (10 mL) was added 2M LiBH₄ in THF (1.14 mL, 2.287 mmol) at 0° C. and the reaction mixture stirred for 2 h at 0° C. The progress of the reaction was monitored by TLC and LCMS. Then reaction mixture was quenched with water (10 mL) and extracted with ethyl acetate (2×30 mL). Organic layer was washed with brine solution (10 mL), dried over Na₂SO₄ and concentrated to get crude compound. It was purified combi-flash, compound eluted at 80% Ethyl acetate in pet ether to afford 3-chlorobenzyl ((S)-1-(((S)-5-((1-benzylcyclopropyl)amino)-1-hydroxy-5-oxopentan-2-yl)amino)-3-cyclohexyl-1-oxopropan-2-yl)carbamate (7) TLC system: 5% Methanol in DCM Rf: 0.2 LCMS (ESI): m/z 584.53 (M+H)⁺

### 3-Chlorobenzyl ((S)-1-(((S)-5-((1-benzylcyclopropyl)amino)-1,5-dioxopentan-2-yl)amino)-3-cyclohexyl-1-oxopropan-2-yl)carbamate (Compound C33)

To a stirred solution of 3-chlorobenzyl ((S)-1-(((S)-5-((1-benzylcyclopropyl)amino)-1-hydroxy-5-oxopentan-2-yl)amino)-3-cyclohexyl-1-oxopropan-2-yl)carbamate (7) (200 mg, 0.343 mmol) was dissolved in ethyl acetate (10 mL) was added Dess-Martin periodinane (290 mg, 0.686 mmol) at 0° C. and stirred at RT for 3 h. Reaction mixture was diluted with ethyl acetate (10 mL) and washed with sat. Hypo solution (3×10 mL), sat. NaHCO₃ solution (3×20 mL). Organic layer was dried over anhydrous Na₂SO₄, filtered and concentrated to afford crude residue. It was purified by prep HPLC to afford 3-chlorobenzyl ((S)-1-(((S)-5-((1-benzylcyclopropyl) amino)-1,5-dioxopentan-2-yl)amino)-3-cyclohexyl-1-oxopropan-2-yl)carbamate (Compound C33). TLC system: 80% Ethyl acetate in Pet ether Rf: 0.4

### Example 47: Synthesis of Compounds C43 and C61

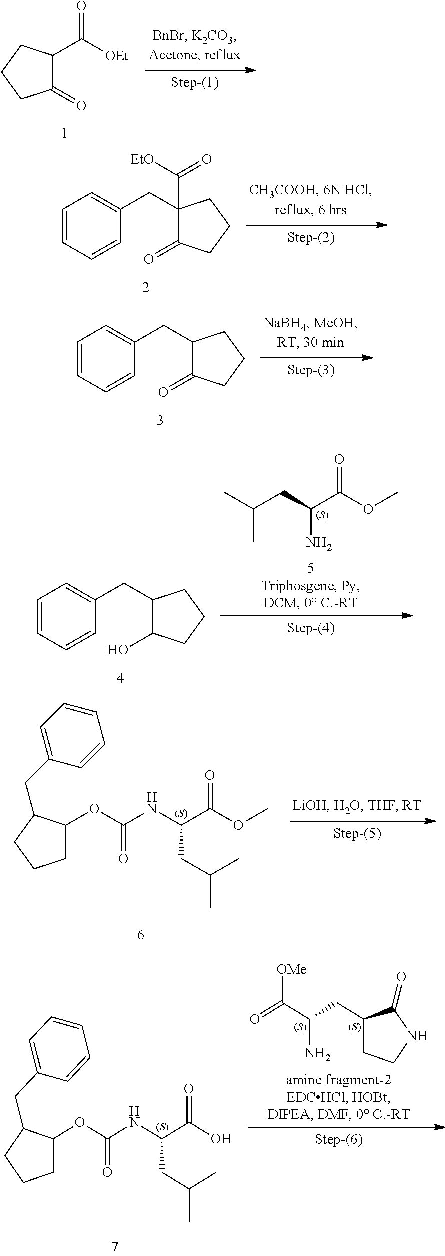

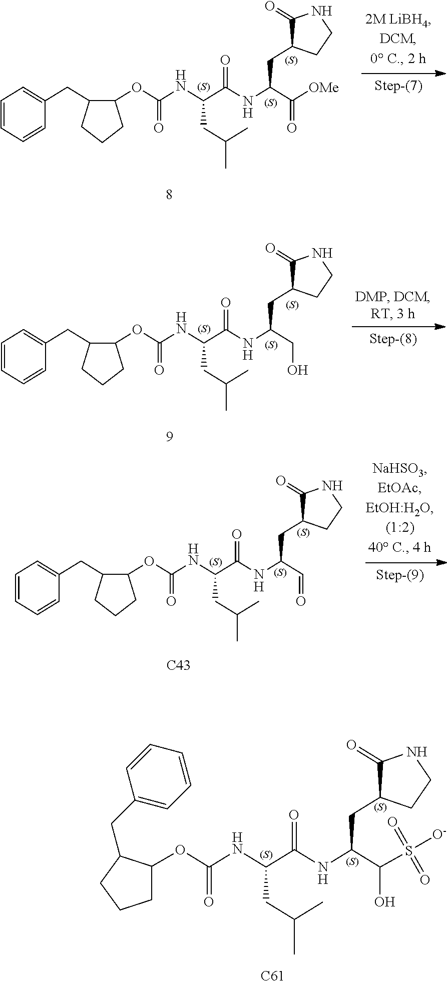

### Ethyl 1-benzyl-2-oxocyclopentane-1-carboxylate (2)

To a stirred solution of K₂CO₃ (17.6 g, 128.205 mmol) in Acetone (100 mL) was added ethyl 2-oxocyclopentane-1-carboxylate (1) (10 g, 64.102 mmol) at RT, followed by benzyl bromide (7.6 mL, 64.102 mmol) at RT and the reaction mixture was refluxed at 70° C. for 16 h. The progress of the reaction was monitored by TLC. Reaction mixture was cooled to RT and quenched with saturated NaHCO₃ (150 mL) then extracted with ethyl acetate (2×100 mL) then washed with the ethyl acetate layer with saturated NaHCO₃ (3×100 mL), dried over sodium sulfate and evaporated under reduced pressure. The crude residue was purified by silica gel column by eluting with 3% ethyl acetate in hexane to afford ethyl 1-benzyl-2-oxocyclopentane-1-carboxylate. TLC system: 10% Ethyl acetate in Hexane Rf: 0.5 LCMS (ESI): m/z=247.26 [M+H]⁺

### 2-benzylcyclopentan-1-one (3)

To a stirred solution of ethyl 1-benzyl-2-oxocyclopentane-1-carboxylate (2) (15 g, 60.975 mmol) in glacial acetic acid (150 mL) was added 6N aq.HCl (75 mL) at RT and the reaction mixture was refluxed at 70° C. for 6 h. The progress of the reaction was monitored by TLC. Reaction mixture was cooled to RT and poured into ice-cold water (100 mL) and extracted with ethyl acetate (3×50 mL), dried over sodium sulfate and evaporated under reduced pressure. The crude residue was purified by silica gel column by eluting with 2% ethyl acetate in hexane to afford 2-benzylcyclopentan-1-one (3). TLC system: 5% Ethyl acetate in Hexane Rf: 0.5 LCMS (ESI): m/z=175.21[M+H]+

### 2-benzylcyclopentan-1-ol (4)

To a stirred solution of 2-benzylcyclopentan-1-one (3) (5 g, 28.735 mmol) in MeOH (50 mL) was added NaBH₄ (1.06 g, 57.471 mmol) at 0° C. Reaction mixture was allowed to stir at RT for 30 min. The progress of the reaction was monitored by TLC. Reaction mixture was distilled and quenched with 1N HCl extracted with ethyl acetate (2×50 mL), dried over sodium sulfate and evaporated under reduced pressure to afford the crude compound 2-benzylcyclopentan-1-ol (4). TLC system: 5% Ethyl acetate in Hexane Rf: 0.2 LCMS (ESI): m/z=171.43 [M+H]+

### Methyl (((2-benzylcyclopentyl) oxy)carbonyl)-L-leucinate (6)

To a stirred solution of 2-benzylcyclopentan-1-ol (4) (5 vg, 28.409 mmol) in DCM (50 mL) was added pyridine (2.5 mL, 28.409 mmol), Triphosgene (2.1 g, 14.204 mmol) at 0° C. slowly, followed by methyl L-leucinate hydrochloride (5) (4.9 g, 34.091 mmol) and stirred at RT for 16 h. The progress of the reaction was monitored by TLC. Reaction mixture was quenched with ice water (50 mL), concentrated the organic layer, again washed with 1N aq HCl solution, then extracted with DCM (2×50 mL), dried over sodium sulfate and evaporated under reduced pressure. The crude residue was purified by silica gel column by eluting with 4% ethyl acetate in hexane to afford methyl (((2-benzylcyclopentyl)oxy)carbonyl)-L-leucinate (6). TLC system: 10% Ethyl acetate in Hexane Rf: 0.5 LCMS (ESI): m/z=348.33 [M+H]+

### (((2-benzylcyclopentyl) oxy) carbonyl)-L-leucine (7)

To a stirred solution of methyl (((2-benzylcyclopentyl) oxy) carbonyl)-L-leucinate (6) (4.5 g, 12.968 mmol) in THF (40 mL), DM water (20 mL), LiOH.H₂O (933 mg, 38.904 mmol) was added. Reaction mixture was stirred at RT for 2 h. The progress of the reaction was monitored by TLC. After consumption of starting material, the reaction mixture was concentrated and acidified with 1N aq. HCl, extracted with ethyl acetate (2×50 mL), dried over sodium sulfate and evaporated under reduced pressure to afford (((2-benzylcyclopentyl)oxy)carbonyl)-L-leucine (7) which was used directly in the next step. TLC system: 5% Methanol in dichloromethane Rf: 0.2 LCMS (ESI): m/z=334.45 [M+H]⁺

### Methyl (2S)-2-((2S)-2-((((2-benzylcyclopentyl) oxy) carbonyl) amino)-4-methylpentanamido)-3-((S)-2-oxopyrrolidin-3-yl) propanoate (8)

To a stirred solution of (((2-benzylcyclopentyl)oxy)carbonyl)-L-leucine (7) (2 g, 6.006 mmol), in DMF (20 mL) was added EDC.HCl (1.72 g, 9.009 mmol), HOBT (1.21 g, 9.009 mmol), DIPEA (3.13 mL, 18.018 mmol) and methyl (2S)-2-amino-3-(2-oxopyrrolidin-3-yl)propanoate (amine-fragment-2) (1.34 g, 7.207 mmol) at 0° C. simultaneously and stirred at room temperature for 16 h. Reaction mixture was diluted with ice water (100 mL), extracted with ethyl acetate (2×100 mL), dried over sodium sulfate and evaporated under reduced pressure. The crude residue was purified by silica gel column by eluting with 5% methanol in dichloromethane to afford methyl (2S)-2-((2S)-2-((((2-benzylcyclopentyl)oxy)carbonyl)amino)-4-methylpentanamido)-3-((S)-2-oxopyrrolidin-3-yl)propanoate (8). TLC system: 5% Methanol in dichloromethane Rf: 0.3 LCMS (ESI): m/z=502.68 [M+H]⁺

### 2-benzylcyclopentyl ((S)-1-(((S)-1-hydroxy-3-((S)-2-oxopyrrolidin-3-yl) propan-2-yl) amino)-4-methyl-1-oxopentan-2-yl)carbamate (9)

To a stirred solution of methyl (2S)-2-((2S)-2-((((2-benzylcyclopentyl) oxy)carbonyl) amino)-4-methylpentanamido)-3-((S)-2-oxopyrrolidin-3-yl)propanoate (8) (1 g, 1.990 mmol) in Dichloromethane (10 mL), 2M LiBH₄ in THF (2 mL, 3.981 mmol) was added at 0° C. and stirred for 2 h The progress of the reaction was monitored by TLC and LCMS. the reaction mixture was quenched with saturated NH₄Cl solution and extracted with Dichloromethane (2×50 mL), filtrate was concentrated under reduced 2-benzylcyclopentyl ((S)-1-(((S)-1-hydroxy-3-((S)-2-oxo pyrrolidin-3-yl) propan-2-yl)amino)-4-methyl-1-oxopentan-2-yl)carbamate (9). TLC system: 5% Methanol in dichloromethane Rf: 0.4 LCMS (ESI): m/z=474.84 [M+H]⁺

### 2-benzylcyclopentyl ((S)-4-methyl-1-oxo-1-(((S)-1-oxo-3-((S)-2-oxopyrrolidin-3-yl) propan-2-yl) amino) pentan-2-yl) carbamate (Compound C43)

To a stirred solution of 2-benzylcyclopentyl ((S)-1-(((S)-1-hydroxy-3-((S)-2-oxo pyrrolidin-3-yl) propan-2-yl)amino)-4-methyl-1-oxopentan-2-yl) carbamate (9) (0.2 g, 0.422 mmol) was dissolved in dichloromethane (10 mL) was added Dess-Martin periodinane (0.537 g, 1.268 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude compound. The crude compound was purified by Prep. HPLC to afford 2-benzylcyclopentyl ((S)-4-methyl-1-oxo-1-(((S)-1-oxo-3-((S)-2-oxopyrrolidin-3-yl) propan-2-yl) amino) pentan-2-yl) carbamate (Compound C43). TLC system: 10% Methanol in dichloromethane Rf: 0.5 LCMS (ESI): m/z=472.62 (M+H)⁺

### (2S)-2-((2S)-2-((((2-benzylcyclopentyl)oxy)carbonyl)amino)-4-methylpentanamido)-1-hydroxy-3-((S)-2-oxopyrrolidin-3-yl)propane-1-sulfonate (Compound C61)

To a stirred solution of 2-benzylcyclopentyl ((S)-4-methyl-1-oxo-1-(((S)-1-oxo-3-((S)-2-oxopyrrolidin-3-yl)propan-2-yl)amino)pentan-2-yl)carbamate (Compound C43) (80 mg, 0.169 mmol) in ethanol (2 mL), EtOAc (5 mL), water (1 mL) was added NaHSO₃ (22 mg, 0.219 mmol) at RT and heated to 50° C. for 16 h. The progress of the reaction was monitored by TLC and LCMS. After 16 h, the reaction mixture was cooled to RT and filtered through celite pad then washed with ethanol (5 mL). Filtrate was evaporated under reduce pressure to afford crude residue. This residue was triturated with diethyl ether (2×5 mL), EtOAc (2×5 mL), the solvents were decanted, the solid was dried well to afford (2S)-2-((2S)-2-((((2-benzylcyclopentyl)oxy)carbonyl) amino)-4-methylpentanamido)-1-hydroxy-3-((S)-2-oxopyrrolidin-3-yl)propane-1-sulfonate (Compound C61). TLC system: 5% Methanol in DCM R_{f}: 0.3 LCMS (ESI): m/z 552.2 [M−H]−

### Example 48: Synthesis of Compounds C54 and C69

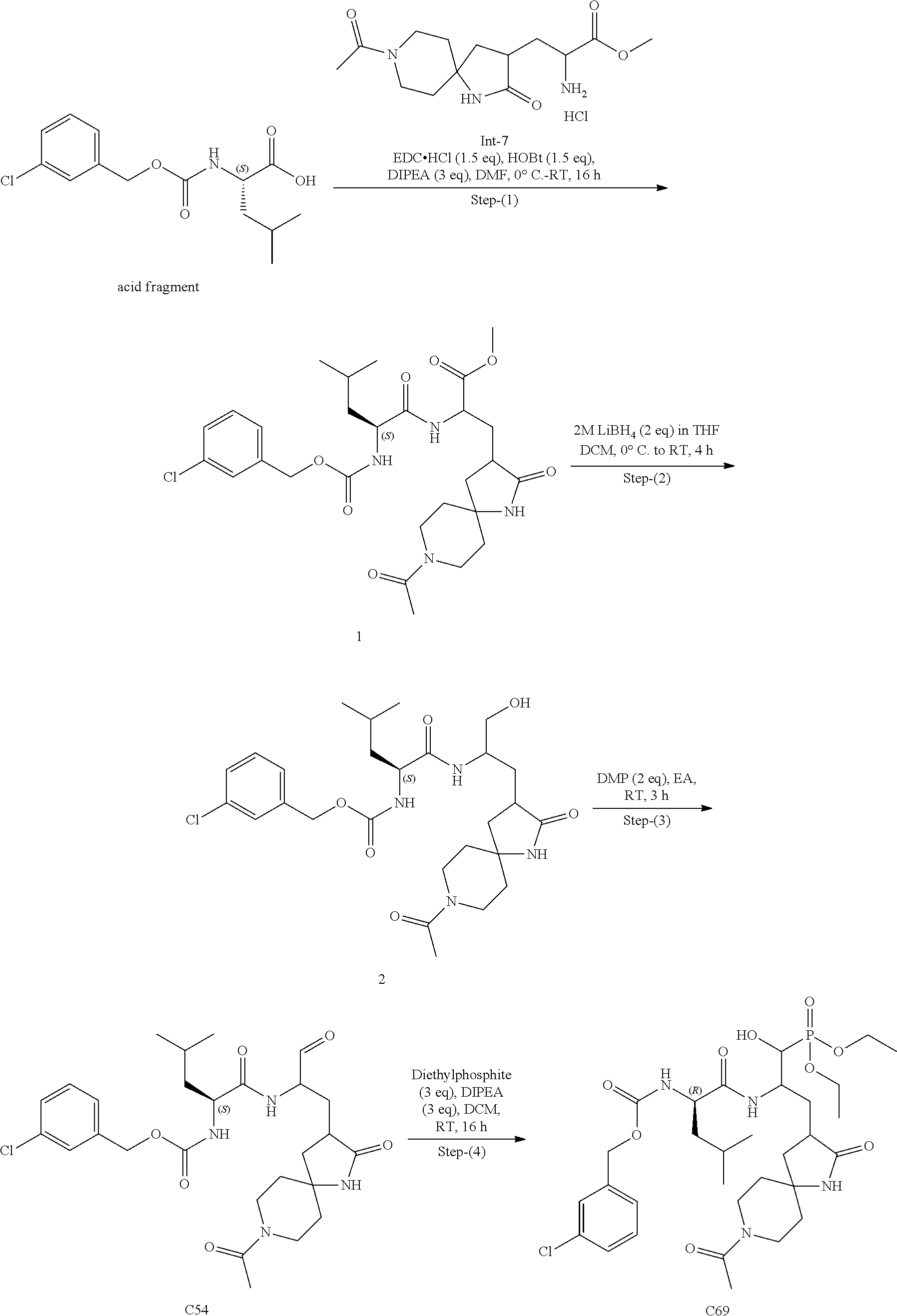

### Methyl 3-(8-acetyl-2-oxo-1,8-diazaspiro[4.5]decan-3-yl)-2-((S)-2-((((3-chlorobenzyl)oxy)carbonyl)amino)-4-methylpentanamido)propanoate (1)

To a stirred solution of (((3-chlorobenzyl)oxy)carbonyl)-L-leucine acid (Acid fragment) (270 mg, 0.903 mmol) in DMF (5 mL) at 0° C. was added EDC.HCl (259 mg, 1.35 mmol), HOBT (183 mg, 1.35 mmol), DIPEA (0.5 mL, 2.7 mmol) and ethyl 3-(8-acetyl-2-oxo-1,8-diazaspiro[4.5]decan-3-yl)-2-aminopropanoate hydrochloride (Int-7) (345 mg, 0.99 mmol) simultaneously and stirred at room temperature for 16 h. The progress of the reaction was monitored by TLC and LCMS. After 16 h, reaction mixture was quenched with ice water (20 mL), extracted with ethyl acetate (2×30 mL), the combined organic layer was dried over sodium sulfate and evaporated under reduced pressure. The crude residue was purified by silica gel column by eluting with 50% ethyl acetate in pet ether to afford methyl 3-(8-acetyl-2-oxo-1,8-diazaspiro[4.5]decan-3-yl)-2-((S)-2-((((3-chlorobenzyl)oxy)carbonyl)amino)-4-methylpentanamido)propanoate (1). TLC system: 5% Methanol in DCM R_{f}: 0.6 LCMS (ESI): m/z 593.59 (M+H)⁺

### 3-Chlorobenzyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2)

To a stirred solution of methyl 3-(8-acetyl-2-oxo-1,8-diazaspiro[4.5]decan-3-yl)-2-((S)-2-((((3-chlorobenzyl)oxy)carbonyl)amino)-4-methylpentanamido)propanoate (1) (280 mg, 0.47 mmol) in DCM (3 mL) was added 2M LiBH₄ in THF (0.4 mL, 0.94 mmol) at 0° C. and the reaction mixture stirred for 2 h at RT. The progress of the reaction was monitored by TLC and LCMS. After 4 h, reaction mixture was quenched with water (20 mL) and extracted with DCM (2×30 mL). Organic layer was washed with brine solution (30 mL), and combined organic layer was dried over Na₂SO₄ and concentrated to afford the

### 3-chlorobenzyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2). TLC system: 5% MeOH in DCM R_{f} 0.3 LCMS (ESI): m/z 551.38 (M+H)⁺

### 3-Chlorobenzyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C54)

To a stirred solution of 3-chlorobenzyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2) (120 mg, 0.26 mmol) in ethyl acetate (5 mL) was added Dess-Martin periodinane (479 mg, 1.13 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 3-chlorobenzyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C54). TLC system: 5% Methanol in DCM R_{f}: 0.35 LCMS (ESI): m/z 549.50 (M+H)⁺

### 3-Chlorobenzyl ((2R)-1-((3-(8-acetyl-2-oxo-1,8-diazaspiro[4.5]decan-3-yl)-1-(diethoxyphosphoryl)-1-hydroxypropan-2-yl)amino)-4-methyl-1-oxopentan-2-yl)carbamate (Compound C69)

To a stirred solution of 3-chlorobenzyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C54) (200 mg, 0.36 mmol) in DCM (2 mL) was added DIPEA (0.2 mL, 1.09 mmol), diethyl phosphite (0.1 mL, 0.72 mmol) at 0° C. and stirred at RT for 16 h. The progress of the reaction was monitored by TLC and LCMS. Quenched with water (20 mL) and extracted with DCM (2×30 mL). Organic layer was washed with brine solution (30 mL), and combined organic layer was dried over Na₂SO₄. Crude was purified by prep HPLC to afford 3-chlorobenzyl ((2R)-1-((3-(8-acetyl-2-oxo-1,8-diazaspiro[4.5]decan-3-yl)-1-(diethoxyphosphoryl)-1-hydroxypropan-2-yl)amino)-4-methyl-1-oxopentan-2-yl)carbamate (Compound C69). TLC system: 5% Methanol in DCM R_{f}: 0.5 LCMS (ESI): m/z 587.2 (M+H)⁺

### Example 49: Synthesis of Compounds C56 and C63

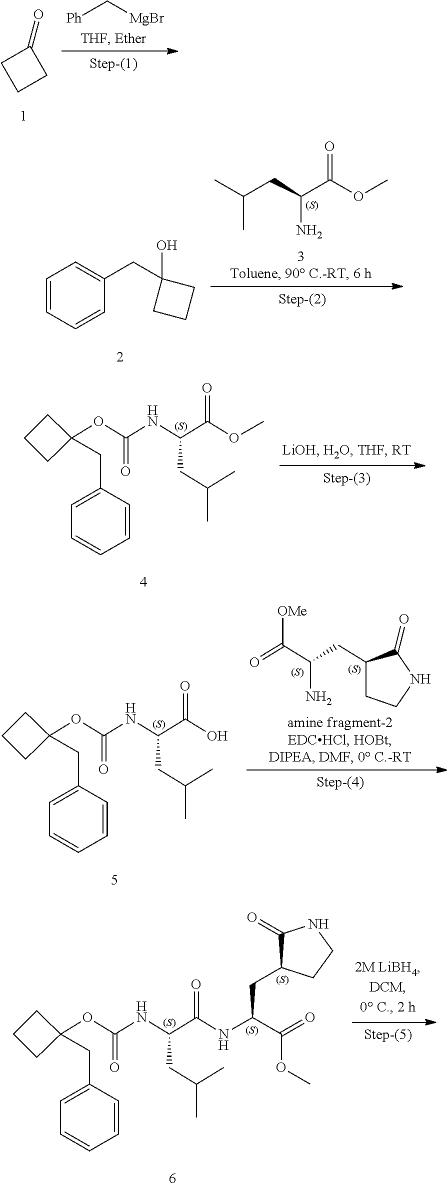

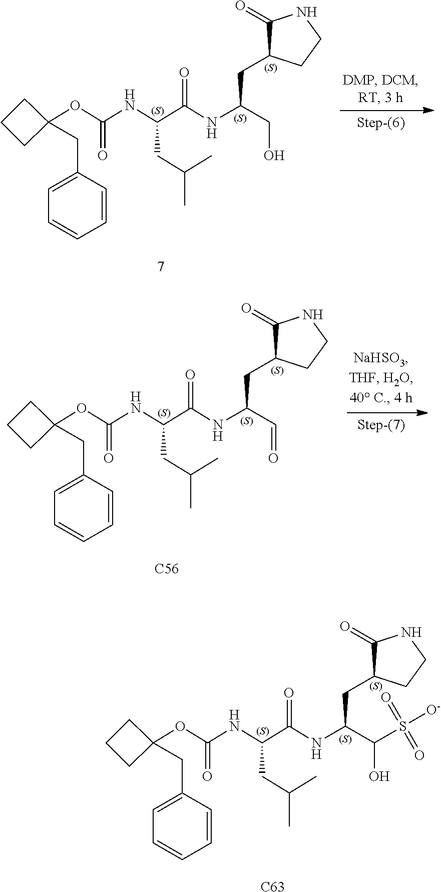

### 1-benzylcyclobutan-1-ol (2)

To a stirred solution of cyclobutanone (1) (5 g, 71.428 mmol) in THF (50 mL) was added phenyl magnesium bromide (71 mL, 142.85 mmol) at 0° C. and the reaction mixture was stirred at RT for 16 h. The progress of the reaction was monitored by TLC. Reaction mixture was quenched with saturated NH₄Cl (150 mL) and filtered through celite pad and washed with ethyl acetate (200 mL), dried over sodium sulfate and evaporated under reduced pressure. The crude residue was purified by silica gel column by eluting with 3% ethyl acetate in hexane to afford 1-benzylcyclobutan-1-ol (2). TLC system: 5% Ethyl acetate in Hexane Rf: 0.4 LCMS (ESI): m/z=163.10 [M+H]⁺

### Methyl ((1-benzylcyclobutoxy)carbonyl)-L-Leucinate (4)

To a stirred solution of 1-benzylcyclobutan-1-ol (2) (3 g, 18.518 mmol) in toluene (30 mL) was added methyl (S)-2-isocyanato-4-methylpentanoate (3) (3.79 g, 22.222 mmol) and stirred at 90° C. for 16 h. The progress of the reaction was monitored by TLC. Reaction mixture was cooled to RT and evaporated under reduced pressure. The crude residue was purified by silica gel column by eluting with 2% ethyl acetate in hexane to afford methyl ((1-benzylcyclobutoxy)carbonyl)-L-leucinate (4). TLC system: 5% Ethyl acetate in Hexane Rf: 0.6 LCMS (ESI): m/z=334.38 [M+H]

### ((1-benzylcyclobutoxy)carbonyl)-L-leucine (5)

To a stirred solution of methyl ((1-benzylcyclobutoxy)carbonyl)-L-leucinate (4) (3.5 g, 10.510 mmol) in THF (40 mL), DM water (20 mL), LiOH.H₂O (756 mg, 31.531 mmol) was added. Reaction mixture was stirred at RT for 2 h. The progress of the reaction was monitored by TLC. After consumption of starting material, the reaction mixture was concentrated and acidified with 1N HCl, extracted with ethyl acetate (2×50 mL), dried over sodium sulfate and evaporated under reduced pressure to afford ((1-benzylcyclobutoxy)carbonyl)-L-leucine (5) which was used directly in the next step. TLC system: 5% Methanol in dichloromethane Rf: 0.5 LCMS (ESI): m/z=320.36 [M+H]⁺

### Methyl (S)-2-((S)-2-(((1-benzylcyclobutoxy)carbonyl)amino)-4-methylpentanamido)-3-((S)-2-oxopyrrolidin-3-yl)propanoate (6)

To a stirred solution of ((1-benzylcyclobutoxy)carbonyl)-L-leucine (5) (3 g, 9.803 mmol) in DMF (30 mL) was added EDC.HCl (2.8 g, 14.705 mmol), HOBT (1.9 g, 14.705 mmol), DIPEA (5.4 mL, 29.411 mmol) and methyl (2S)-2-amino-3-(2-oxopyrrolidin-3-yl)propanoate (amine-fragment-2) (2.1 g, 11.764 mmol) at 0° C. simultaneously and stirred at room temperature for 16 h. Reaction mixture was diluted with ice water (100 mL), extracted with ethyl acetate (2×100 mL), dried over sodium sulfate and evaporated under reduced pressure. The crude residue was purified by silica gel column by eluting with 5% methanol in dichloromethane to afford methyl (S)-2-((S)-2-(((1-benzylcyclobutoxy)carbonyl)amino)-4-methylpentanamido)-3-((S)-2-oxopyrrolidin-3-yl)propanoate (6). TLC system: 10% Methanol in dichloromethane Rf: 0.4 LCMS (ESI): m/z=448.18 [M+H]⁺

### 1-benzylcyclobutyl ((S)-1-(((S)-1-hydroxy-3-((S)-2-oxopyrrolidin-3-yl)propan-2-yl)amino)-4-methyl-1-oxopentan-2-yl)carbamate (7)

To a stirred solution of methyl (S)-2-((S)-2-(((1-benzyl cyclobutoxy) carbonyl) amino)-4-methyl pentanamido)-3-((S)-2-oxopyrrolidin-3-yl) propanoate (6) (400 mg, 0.871 mmol) in Dichloromethane (10 mL), 2M LiBH₄ in THF (1.1 mL, 2.614 mmol) was added at 0° C. and stirred for 2 h The progress of the reaction was monitored by TLC and LCMS. the reaction mixture was quenched with saturated NH₄Cl solution and extracted with Dichloromethane (2×50 mL), filtrate was concentrated under reduced 1-benzylcyclobutyl ((S)-1-(((S)-1-hydroxy-3-((S)-2-oxopyrrolidin-3-yl)propan-2-yl)amino)-4-methyl-1-oxopentan-2-yl)carbamate (7). TLC system: 10% Methanol in dichloromethane Rf: 0.5 LCMS (ESI): m/z=458.52 [M+H]⁺

### 1-Benzylcyclobutyl ((S)-4-methyl-1-oxo-1-(((S)-1-oxo-3-((S)-2-oxopyrrolidin-3-yl)propan-2-yl)amino)pentan-2-yl)carbamate (Compound C56)

To a stirred solution of 1-benzylcyclobutyl ((S)-1-(((S)-1-hydroxy-3-((S)-2-oxopyrrolidin-3-yl)propan-2-yl)amino)-4-methyl-1-oxopentan-2-yl)carbamate (7) (0.2 g, 0.435 mmol) was dissolved in dichloromethane (10 mL) was added Dess-Martin periodinane (0.554 g, 1.307 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude product, afford to 1-benzylcyclobutyl ((S)-4-methyl-1-oxo-1-(((S)-1-oxo-3-((S)-2-oxopyrrolidin-3-yl)propan-2-yl)amino)pentan-2-yl)carbamate (Compound C56). TLC system: 10% Methanol in dichloromethane Rf: 0.5 LCMS (ESI): m/z=458.2 (M+H)⁺

### (2S)-2-((S)-2-(((1-Benzylcyclobutoxy)carbonyl)amino)-4-methylpentanamido)-1-hydroxy-3-((S)-2-oxopyrrolidin-3-yl)propane-1-sulfonate (Compound C63)

To a stirred solution of crude 1-benzylcyclobutyl ((S)-4-methyl-1-oxo-1-(((S)-1-oxo-3-((S)-2-oxopyrrolidin-3-yl)propan-2-yl)amino)pentan-2-yl)carbamate (Compound C56) (180 mg, 0.393 mmol) in EtOAc (5 mL) was added EtOH/_{H2O} (2 mL, 1 mL) and NaHSO3 (61 mg, 0.590 mmol) at RT and the RM was stirred at 40° C. for 16 h. The progress of the reaction was monitored by TLC and LCMS. Reaction mixture was filtered through celite pad and washed the celite pad with EtOH (20 mL) to afforded (2S)-2-((S)-2-(((1-benzylcyclobutoxy)carbonyl)amino)-4-methylpentanamido)-1-hydroxy-3-((S)-2-oxopyrrolidin-3-yl)propane-1-sulfonate (Compound C63). TLC system: 10% Methanol in dichloromethane Rf: 0.4 LCMS (ESI): m/z=538.2 (M+H)⁺

### Example 50: Synthesis of Compounds C58 and C59

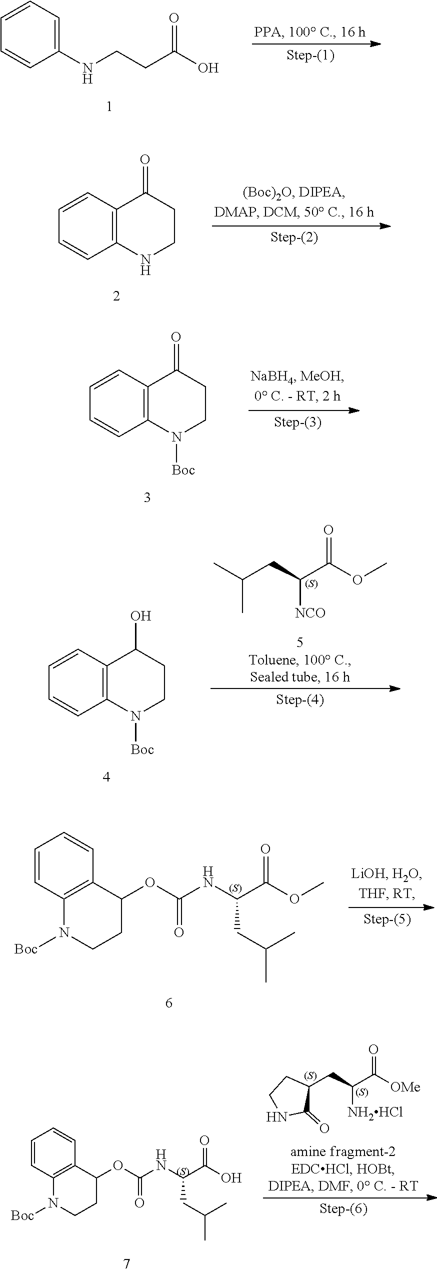

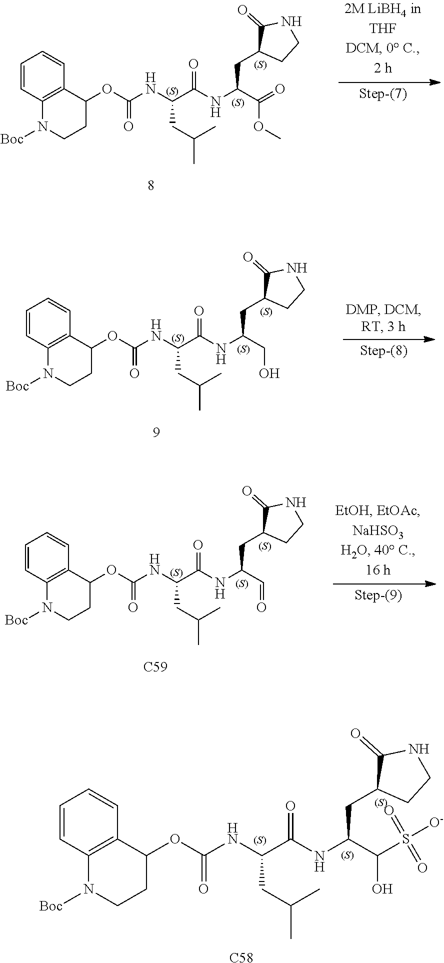

### 2,3-Dihydroquinolin-4(1H)-one (2)

To a stirred solution of 3-(phenylamino) propanoic acid (1) (20 g, 121.21 mmol) in PPA (200 g) was heated to 100° C. for 16 h. The progress of the reaction was monitored by TLC. Reaction mixture was diluted with ice cold water and extracted with EtOAc (3×100 mL). Combined the organic layer and washed with brine (100 mL), dried over sodium sulfate, concentrated under reduced pressure to afford crude 2,3-dihydroquinolin-4(1H)-one (2) which was used directly in the next step. TLC system: 50% Ethyl acetate in hexane Rf: 0.5 LCMS (ESI): m/z 148.07 [M+H]⁺

### Tert-butyl 4-oxo-3,4-dihydroquinoline-1(2H)-carboxylate (3)

To a stirred solution of 2,3-dihydroquinolin-4(1H)-one (2) (5.4 g, 36.73 mmol) in dichloromethane (80 mL) was added DIPEA (7.5 mL, 43.78 mmol), (Boc)₂O (9.5 mL, 43.78 mmol), DMAP (445 mg, 3.64 mmol) at RT and heated to 50° C. for 16 h. The progress of the reaction was monitored by TLC. Reaction mixture was diluted with DCM and washed with water (2×50 mL), dried over sodium sulfate, concentrated under reduced pressure to afford crude residue, crude residue was purified by normal phase chromatography to afford tert-butyl 4-oxo-3,4-dihydroquinoline-1(2H)-carboxylate (3). TLC system: 40% Ethyl acetate in hexane Rf: 0.7 LCMS (ESI): m/z 248.12 [M+H]⁺

### Tert-butyl 4-hydroxy-3,4-dihydroquinoline-1(2H)-carboxylate (4)

To a stirred solution of tert-butyl 4-oxo-3,4-dihydroquinoline-1(2H)-carboxylate (3) (4 g, 16.12 mmol) in methanol (40 mL) was added NaBH₄ (900 mg, 24.19 mmol) at 0° C. and stirred at RT for 2 h. The progress of the reaction was monitored by TLC. Reaction mixture was quenched with sat. NH₄Cl solution and evaporated under reduced pressure to afford crude, this crude was diluted with water and extracted with EtOAc (2×50 mL), dried over sodium sulfate, concentrated under reduced pressure to afford crude residue, crude residue was purified by normal phase chromatography to afford tert-butyl 4-hydroxy-3,4-dihydroquinoline-1(2H)-carboxylate (4). TLC system: 40% Ethyl acetate in hexane Rf: 0.3 LCMS (ESI): m/z 272.30 [M+Na]⁺

### Tert-butyl 4-((((S)-1-methoxy-4-methyl-1-oxopentan-2-yl) carbamoyl) oxy)-3,4-dihydroquinoline-1(21-1)-carboxylate (6)

To a stirred solution of Tert-butyl 4-hydroxy-3,4-dihydroquinoline-1(2H)-carboxylate (4) (2 g, 8.03 mmol) in toluene (20 mL) was added methyl (S)-2-isocyanato-4-methylpentanoate (5) (2.06 g, 12.04 mmol) at RT and heated to 100° C. under sealed tube for 16 h. The progress of the reaction was monitored by TLC. Reaction mixture was evaporated under reduced pressure to afford crude residue, crude residue was purified by normal phase chromatography to afford tert-butyl 4-((((S)-1-methoxy-4-methyl-1-oxopentan-2-yl) carbamoyl) oxy)-3,4-dihydroquinoline-1(2H)-carboxylate (6). TLC system: 30% Ethyl acetate in hexane Rf: 0.6 LCMS (ESI): m/z 443.44 [M+Na]⁺

### (((1-(cert-butoxycarbonyl)-1, 2, 3, 4-tetrahydroquinolin-4-yl) oxy) carbonyl)-L-leucine (7)

To a stirred solution of tert-butyl 4-((((S)-1-methoxy-4-methyl-1-oxopentan-2-yl)carbamoyl)oxy)-3,4-dihydroquinoline-1(2H)-carboxylate (6) (2.4 g, 5.71 mmol) in THF (15 mL), water (5 mL) was added lithium hydroxide (411 mg, 17.14 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford (((1-(tert-butoxycarbonyl)-1,2,3,4-tetrahydroquinolin-4-yl)oxy)carbonyl)-L-leucine (7). TLC system: 100% EtOAc Rf: 0.1 LCMS (ESI): m/z 429.21 [M+Na]⁺

### Tert-butyl 4-((((S)-1-(((S)-1-methoxy-1-oxo-3-((S)-2-oxopyrrolidin-3-yl) propan-2-yl) amino)-4-methyl-1-oxopentan-2-yl) carbamoyl) oxy)-3,4-dihydroquinoline-1(2H)-carboxylate (8)

To a stirred solution of (((1-(tert-butoxycarbonyl)-1,2,3,4-tetrahydroquinolin-4-yl)oxy)carbonyl)-L-leucine (7) (2 g, 4.92 mmol) DMF (20 mL) added EDC.HCl (1.4 g, 7.38 mmol), HOBt (0.9 g, 7.38 mmol), DIPEA (2.5 mL, 14.76 mmol) and methyl (S)-2-amino-3-((S)-2-oxopyrrolidin-3-yl)propanoate hydrochloride (amine fragment-2) (1.1 g, 5.91 mmol) at 0° C. simultaneously and stirred at room temperature for 16 h. Reaction mixture was diluted with ice water (80 mL), extracted with ethyl acetate (2×50 mL), dried over sodium sulfate and evaporated under reduced pressure. The crude residue was purified by combi-flash NP, compound eluted at 3% methanol in dichloromethane to afford tert-butyl 4-((((S)-1-(((S)-1-methoxy-1-oxo-3-((S)-2-oxopyrrolidin-3-yl)propan-2-yl)amino)-4-methyl-1-oxopentan-2-yl)carbamoyl)oxy)-3,4-dihydroquinoline-1(2H)-carboxylate (8). TLC system: 10% Methanol in dichloromethane Rf: 0.4 LCMS (ESI): m/z 575.54 [M+H]⁺

### Tert-butyl 4-((((S)-1-(((S)-1-hydroxy-3-((S)-2-oxopyrrolidin-3-yl) propan-2-yl) amino)-4-methyl-1-oxopentan-2-yl) carbamoyl) oxy)-3,4-dihydroquinoline-1(2H)-carboxylate (9)

To a stirred solution of tert-butyl 4-((((S)-1-(((S)-1-methoxy-1-oxo-3-((S)-2-oxopyrrolidin-3-yl) propan-2-yl)amino)-4-methyl-1-oxopentan-2-yl)carbamoyl)oxy)-3,4-dihydroquinoline-1(2H)-carboxylate (8) (500 mg, 0.87 mmol) in DCM (10 mL) was added 2M LiBH₄ in THF (0.8 mL, 1.74 mmol) at 0° C. and the reaction mixture stirred for 2 h at 0° C. The progress of the reaction was monitored by TLC and LCMS. Reaction mixture was quenched with sat. Ammonium chloride solution (20 mL) and extracted with DCM (2×20 mL). Organic layer was washed with brine solution (30 mL), dried over Na₂SO₄ and concentrated to afford tert-butyl 4-((((S)-1-(((S)-1-hydroxy-3-((S)-2-oxopyrrolidin-3-yl)propan-2-yl)amino)-4-methyl-1-oxopentan-2-yl)carbamoyl)oxy)-3,4-dihydroquinoline-1(2H)-carboxylate (9). TLC system: 10% Methanol in dichloromethane Rf: 0.2 LCMS (ESI): m/z 569.60 [M+Na]⁺

### Tert-butyl 4-((((S)-4-methyl-1-oxo-1-(((S)-1-oxo-3-((S)-2-oxopyrrolidin-3-yl) propan-2-yl)amino)pentan-2-yl)carbamoyl)oxy)-3,4-dihydroquinoline-1(2H)-carboxylate (Compound C59)

To a stirred solution of tert-butyl 4-((((S)-1-(((S)-1-hydroxy-3-((S)-2-oxopyrrolidin-3-yl) propan-2-yl) amino)-4-methyl-1-oxopentan-2-yl)carbamoyl)oxy)-3,4-dihydroquinoline-1(2H)-carboxylate (9) (150 mg, 0.27 mmol) was dissolved in dichloromethane (5 mL) was added Dess-Martin periodinane (349 mg, 0.82 mmol) at 0° C. and stirred at RT for 3 h. Reaction mixture was diluted with DCM (20 mL) followed by sat. Hypo solution (3×20 mL), followed by sat. NaHCO₃ solution (3×20 mL). Organic layer was dried over anhydrous Na₂SO₄, filtered and concentrated to get crude compound. The crude compound was purified by prep HPLC to afford tert-butyl 4-((((S)-4-methyl-1-oxo-1-(((S)-1-oxo-3-((S)-2-oxopyrrolidin-3-yl)propan-2-yl)amino)pentan-2-yl)carbamoyl)oxy)-3,4-dihydroquinoline-1(21-1)-carboxylate (Compound C59). TLC system: 10% Methanol in dichloromethane Rf: 0.5 LCMS (ESI): m/z 545.45 (M+H)⁺

### (2S)-2-((2S)-2-((((1-(tert-butoxycarbonyl)-1, 2, 3, 4-tetrahydroquinolin-4-yl) oxy) carbonyl) amino)-4-methylpentanamido)-1-hydroxy-3-((S)-2-oxopyrrolidin-3-yl) propane-1-sulfonate (Compound C58)

To a stirred solution of tert-butyl 4-((((S)-4-methyl-1-oxo-1-(((S)-1-oxo-3-((S)-2-oxopyrrolidin-3-yl) propan-2-yl)amino)pentan-2-yl) carbamoyl)oxy)-3,4-dihydroquinoline-1((2H)-carboxylate (Compound C59) (0.15 g, 0.27 mmol) was dissolved in ethyl acetate (3.0 mL), ethanol (2.0 mL), water (0.5 mL), and then NaHSO₃ (46 mg, 0.55 mmol) was added and stirred at 40° C. for 16 h. The progress of the reaction was monitored by TLC and LCMS. The reaction mixture was filtered through celite pad and filtrate was dried over sodium sulphate, concentrated and the crude compound was washed with EtOAc and diethyl ether to afford (2S)-2-((2S)-2-((((1-(tert-butoxycarbonyl)-1,2,3,4-tetrahydroquinolin-4-yl)oxy)carbonyl)amino)-4-methylpentanamido)-1-hydroxy-3-((S)-2-oxopyrrolidin-3-yl)propane-1-sulfonate (Compound C58). TLC system: 15% Methanol in dichloromethane Rf: 0.2 LCMS (ESI): m/z 625.2 [M]⁻

### Example 51: Synthesis of Compounds C71 and C62

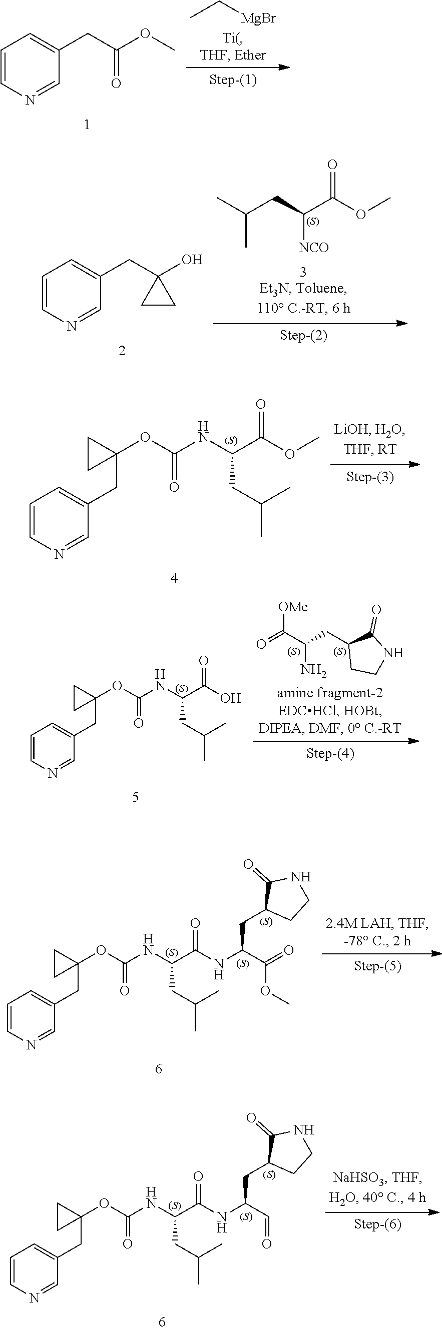

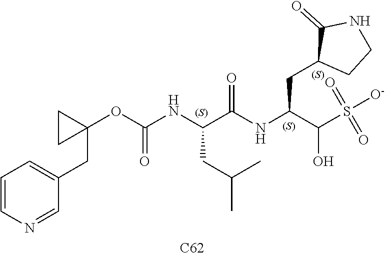

### 1-(Pyridin-3-ylmethyl)cyclopropan-1-ol (2)

To a stirred solution of methyl 2-(pyridin-3-yl)acetate (1) (10 g, 66.225 mmol) in THF (100 mL) were added Titanium isopropoxide (27.4 g, 96.549 mmol) and followed by added 2M Ethyl magnesium chloride in THF (115 mL, 231.78 mmol) slowly drop wise for 30 min at −78° C. Then the reaction mixture stirred at RT for 16 h. The progress of the reaction was monitored by TLC and LCMS. The reaction mixture was quenched with sat ammonium chloride solution (50 mL), extracted with ethyl acetate (3×40 mL), washed with brine solution (100 mL), dried over sodium sulfate, filtered and evaporated under reduced pressure. The crude residue was purified by combi-flash compound eluted at 94% Ethyl acetate in pet ether to afford 1-(pyridin-3-ylmethyl)cyclopropan-1-ol (2). TLC system: 80% Ethyl acetate in Hexane Rf: 0.2 LCMS (ESI): m/z=150.06 [M+H]⁺

### Methyl ((1-(pyridin-3-ylmethyl)cyclopropoxy)carbonyl)-L-leucinate (4)

To a stirred solution of 1-(pyridin-3-ylmethyl)cyclopropan-1-ol (2) (3.7 g, 24.832 mmol) in toluene (20 mL) was added Et₃N (10 mL, 74.496 mmol), followed by methyl (S)-2-isocyanato-4-methylpentanoate (3) (6.3 g, 37.248 mmol) and stirred at 110° C. for 16 h. The progress of the reaction was monitored by TLC. Reaction mixture was cooled to RT and evaporated under reduced pressure. The crude residue was purified by silica gel column by eluting with 40% ethyl acetate in hexane to afford methyl ((1-(pyridin-3-ylmethyl)cyclopropoxy)carbonyl)-L-leucinate (4). TLC system: 70% Ethyl acetate in Hexane Rf: 0.6 LCMS (ESI): m/z=321.34 [M+H]⁺

### ((1-(Pyridin-3-ylmethyl)cyclopropoxy)carbonyl)-L-leucine (5)

To a stirred solution of methyl ((1-(pyridin-3-ylmethyl)cyclopropoxy)carbonyl)-L-leucinate (4) (4.8 g, 15.00 mmol) in THF (40 mL), DM water (20 mL), LiOH.H₂O (1.8 g, 45.00 mmol) was added. Reaction mixture was stirred at RT for 2 h. The progress of the reaction was monitored by TLC. After consumption of starting material, the reaction mixture was concentrated and acidified with 1N HCL, extracted with ethyl acetate (2×50 mL), dried over sodium sulfate and evaporated under reduced pressure to afford ((1-(pyridin-3-ylmethyl)cyclopropoxy)carbonyl)-L-leucine (5) which was used directly in the next step. TLC system: 5% Methanol in dichloromethane Rf: 0.5 LCMS (ESI): m/z=307.22 [M+H]⁺

### Methyl (S)-2-((S)-4-methyl-2-(((1-(pyridin-3-ylmethyl) cyclopropoxy) carbonyl) amino) pentanamido)-3-((S)-2-oxopyrrolidin-3-yl) propanoate (6)

To a stirred solution of ((1-(pyridin-3-ylmethyl)cyclopropoxy)carbonyl)-L-leucine (5) (1.5 g, 4.901 mmol) in DMF (15 mL) was added EDC.HCl (1.4 g, 7.352 mmol), HOBT (0.99 g, 7.352 mmol), DIPEA (2.7 mL, 14.705 mmol) and methyl (2S)-2-amino-3-(2-oxopyrrolidin-3-yl)propanoate (amine-fragment-2) (1 g, 5.882 mmol) at 0° C. simultaneously and stirred at room temperature for 16 h. Reaction mixture was diluted with ice water (100 mL), extracted with ethyl acetate (2×100 mL), dried over sodium sulfate and evaporated under reduced pressure. The crude residue was purified by silica gel column by eluting with 5% methanol in dichloromethane to afford methyl (S)-2-((S)-4-methyl-2-(((1-(pyridin-3-ylmethyl)cyclo propoxy) carbonyl) amino) pentanamido)-3-((S)-2-oxopyrrolidin-3-yl)propanoate (6). TLC system: 10% Methanol in dichloromethane Rf: 0.4 LCMS (ESI): m/z=475.14 [M+H]⁺

### 1-(Pyridin-3-ylmethyl)cyclopropyl ((S)-4-methyl-1-oxo-1-(((S)-1-oxo-3-((S)-2-oxopyrrolidin-3-yl)propan-2-yl)amino)pentan-2-yl)carbamate (Compound C71)

To a stirred solution of methyl (S)-2-((S)-4-methyl-2-(((1-(pyridin-3-ylmethyl) cyclopropoxy) carbonyl) amino) pentanamido)-3-((S)-2-oxopyrrolidin-3-yl)propanoate (6) (300 mg, 0.632 mmol) in THF (10 mL), 2.4M LAH in THF (0.3 mL, 0.632 mmol) was added at −78° C. and stirred for 2 h The progress of the reaction was monitored by TLC and LCMS. the reaction mixture was quenched with saturated NH₄Cl solution and extracted with Dichloromethane (2×50 mL), filtrate was concentrated under reduced pressure to afford 1-(pyridin-3-ylmethyl)cyclopropyl ((S)-4-methyl-1-oxo-1-(((S)-1-oxo-3-((S)-2-oxopyrrolidin-3-yl)propan-2-yl)amino)pentan-2-yl)carbamate (Compound C71). TLC system: 10% Methanol in dichloromethane Rf: 0.5 LCMS (ESI): m/z=445.12 [M+H]⁺

### (2S)-1-Hydroxy-2-((S)-4-methyl-2-(((1-(pyridin-3-ylmethyl)cyclopropoxy)carbonyl)amino)pentanamido)-3-((S)-2-oxopyrrolidin-3-yl)propane-1-sulfonate (Compound C62)

To a stirred solution of crude 1-(pyridin-3-ylmethyl)cyclopropyl ((S)-4-methyl-1-oxo-1-(((S)-1-oxo-3-((S)-2-oxopyrrolidin-3-yl)propan-2-yl)amino)pentan-2-yl)carbamate (Compound C71) (200 mg, 0.450 mmol) in EtOAc (5 mL) was added EtOH/H₂O (2 mL, 1 mL) and NaHSO₃ (70 mg, 0.675 mmol) at RT and the RM was stirred at 40° C. for 16 h. The progress of the reaction was monitored by TLC and LCMS. Reaction mixture was filtered through celite pad and washed the celite pad with EtOH (20 mL) to afforded (2S)-1-hydroxy-2-((S)-4-methyl-2-(((1-(pyridin-3-ylmethyl)cyclopropoxy) carbonyl)amino)pentanamido)-3-((S)-2-oxopyrrolidin-3-yl)propane-1-sulfonate (Compound C62). TLC system: 10% Methanol in dichloromethane Rf: 0.4 LCMS (ESI): m/z=525.2 (M+H)⁺

### Example 52: Synthesis of Compound C64

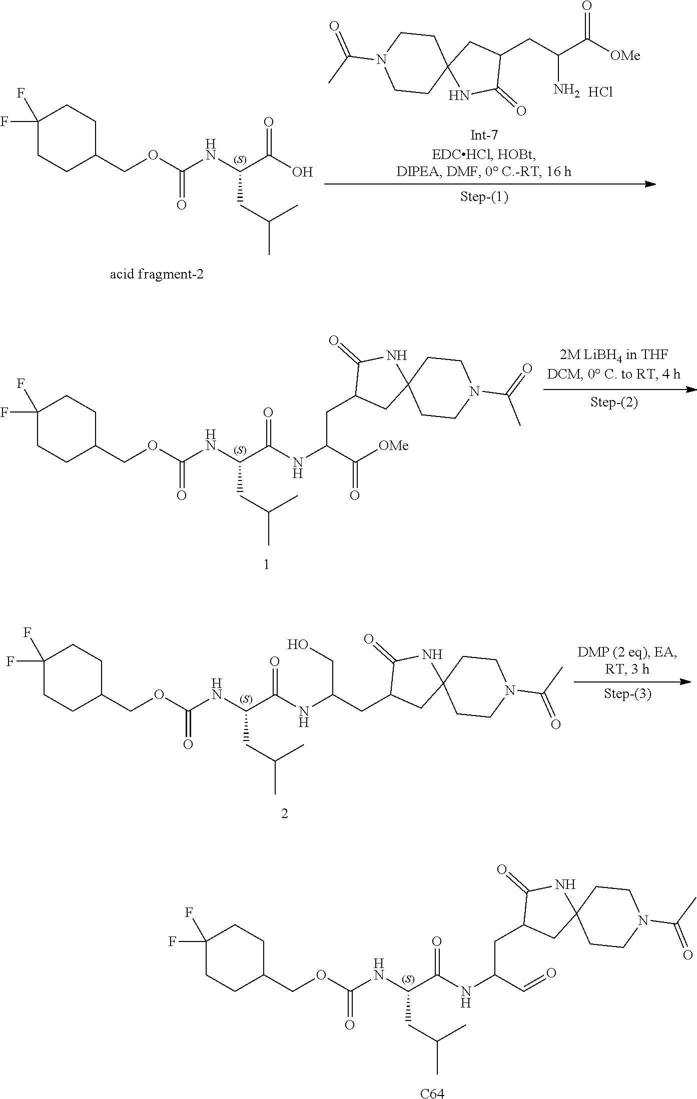

### Methyl 3-(8-acetyl-2-oxo-1,8-diazaspiro[4.5]decan-3-yl)-2-((S)-2-((((4,4-difluorocyclohexyl)methoxy)carbonyl)amino)-4-methylpentanamido)propanoate (1)

To a stirred solution of (((4,4-difluorocyclohexyl)methoxy)carbonyl)-L-leucine (Acid fragment-2) (1.2 g, 3.9 mmol) in DMF (20 mL) at 0° C. was added EDC.HCl (1.1 g, 5.86 mmol), HOBT (0.79 g, 5.86 mmol), DIPEA (2.1 mL, 11.72 mmol) and methyl 3-(8-acetyl-2-oxo-1,8-diazaspiro[4.5]decan-3-yl)-2-aminopropanoate hydrochloride (Int-7) 1.49 g, 4.29 mmol) simultaneously and stirred at room temperature for 16 h. The progress of the reaction was monitored by TLC and LCMS. After 16 h, reaction mixture was quenched with ice water (20 mL), extracted with ethyl acetate (2×100 mL), the combined organic layer was dried over sodium sulfate and evaporated under reduced pressure. The crude residue was purified by silica gel column by eluting with 50% ethyl acetate in pet ether to afford methyl 3-(8-acetyl-2-oxo-1,8-diazaspiro[4.5]decan-3-yl)-2-((S)-2-((((4,4difluorocyclohexyl)methoxy)carbonyl) amino)-4-methylpentanamido)propanoate (1). TLC system: 5% Methanol in DCM R_{f}: 0.65 LCMS (ESI): m/z 601.71 (M+H)⁺

### (4,4-Difluorocyclohexyl)methyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2)

To a stirred solution of methyl 3-(8-acetyl-2-oxo-1,8-diazaspiro[4.5]decan-3-yl)-2-((S)-2-((((4,4difluorocyclohexyl)methoxy)carbonyl) amino)-4-methylpentanamido)propanoate (1) (500 mg, 0.85 mmol) in DCM (3 mL) was added 2M LiBH₄ in THF (0.85 mL, 1.7 mmol) at 0° C. and the reaction mixture stirred for 2 h at RT. The progress of the reaction was monitored by TLC and LCMS. After 4 h, reaction mixture was quenched with water (20 mL) and extracted with DCM (2×30 mL). Organic layer was washed with brine solution (30 mL), and combined organic layer was dried over Na₂SO₄ and concentrated to afford (4,4-difluorocyclohexyl)methyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2). TLC system: 5% MeOH in DCM R_{f} 0.3 LCMS (ESI): m/z 559.68 (M+H)⁺

### (4,4-Difluorocyclohexyl)methyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C64)

To a stirred solution of (4,4-difluorocyclohexyl)methyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2) (400 mg, 0.71 mmol) in ethyl acetate (4 mL) was added Dess-Martin periodinane (1.2 g, 2.87 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford (4,4-difluorocyclohexyl)methyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C64). TLC system: 5% Methanol in DCM R_{f}: 0.3 LCMS (ESI): m/z 557.3 (M+H)⁺

### Example 53: Synthesis of Compounds C96 and C72

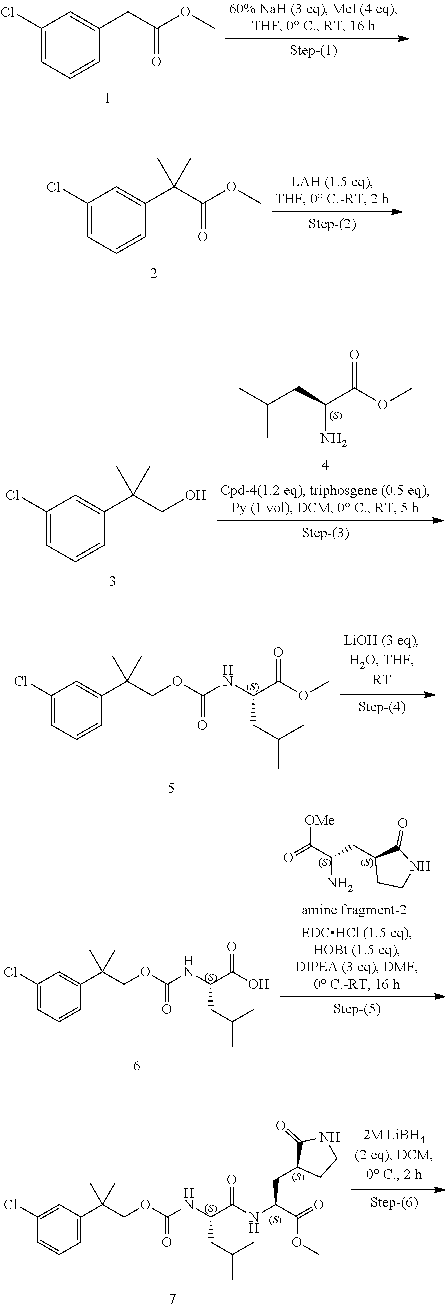

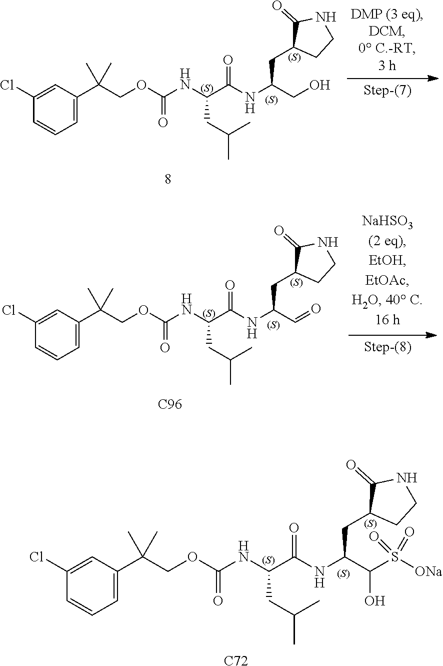

### Methyl 2-(3-chlorophenyl)-2-methylpropanoate (2)

To a stirred solution of methyl 2-(3-chlorophenyl)acetate (1) (10 g, 54.17 mmol) in THF (100 mL) was added 60% NaH (6.5 g, 162.51 mmol) at 0° C. and stirred for 15 min then added Mel (13.5 mL, 216.68 mmol) at same temperature and allowed to RT for 16 h. The progress of the reaction was monitored by TLC, Reaction mixture was quenched with sat. ammonium chloride and extracted with EtOAc (2×100 mL), combined the organic layer and washed with brine solution (100 mL), dried over sodium sulfate, filtered and evaporated under reduced pressure to afford crude, residue was purified by combi-flash, compound eluted at 5% ethyl acetate in pet ether to afford methyl 2-(3-chlorophenyl)-2-methylpropanoate (2). TLC system: 10% Ethyl acetate in Pet ether Rf: 0.3 LCMS (ESI): m/z 213.31 [M+H]⁺

### 2-(3-Chlorophenyl)-2-methylpropan-1-ol (3)

To a stirred solution of methyl 2-(3-chlorophenyl)-2-methylpropanoate (6 g, 28.30 mmol), in THF (80 mL) was added slowly 2.4 M LAH in THF (11.7 mL, 28.30 mmol) at −50° C. and stirred for 1 h. The progress of the reaction was monitored by TLC. Reaction mixture was quenched with sat. ammonium chloride and extracted with EtOAc (2×50 mL), combined the organic layer and washed with brine solution (60 mL), dried over sodium sulfate, filtered and evaporated under reduced pressure to afford crude, residue was purified by combi-flash, compound eluted at 10% ethyl acetate in pet ether to afford 2-(3-chlorophenyl)-2-methylpropan-1-ol (3). TLC system: 30% Ethyl acetate Rf: 0.3 LCMS (ESI): m/z=167.4 [M−OH]+

### Methyl ((2-(3-chlorophenyl)-2-methylpropoxy) carbonyl)-L-leucinate (5)

To a stirred solution of 2-(3-chlorophenyl)-2-methylpropan-1-ol (3) (2.8 g, 15.16 mmol), methyl L-leucinate (2.64 g, 18.19 mmol) in DCM (40 mL) was added pyridine (3 mL, 1 vol) followed by triphosgene (2.24 g, 7.58 mmol) at 0° C. and stirred at room temperature for 3 h. The progress of the reaction was monitored by TLC and LCMS. Reaction mixture was quenched with ice water (30 mL), extracted with DCM (2×30 mL). The combined organic layer was dried over sodium sulfate, filtered and evaporated under reduced pressure. The crude residue was purified by combi-flash, compound eluted at 8% ethyl acetate in pet ether to afford methyl ((2-(3-chlorophenyl)-2-methylpropoxy) carbonyl)-L-leucinate (5). TLC system: 30% Ethyl acetate in Pet ether R_{f}: 0.5 LCMS (ESI): m/z 356.31 [M+H]⁺

### ((2-(3-Chlorophenyl)-2-methylpropoxy) carbonyl)-L-leucine (6)

To a stirred solution of methyl ((2-(3-chlorophenyl)-2-methylpropoxy) carbonyl)-leucinate (5) (2.3 g, 6.47 mmol) in THF (10 mL) and water (5 mL), was added lithium hydroxide (800 mg, 19.43 mmol) at RT and stirred at room temperature for 3 h. The progress of the reaction was monitored by TLC and LCMS. Reaction mixture completely distilled under reduced pressure and diluted with and washed with diethyl ether (20 mL), aq layer was acidified with aq. 1N HCl solution up to pH˜4, and extracted with dichloromethane (2×30 mL), dried over sodium sulfate, concentrated under reduced pressure to afford ((2-(3-chlorophenyl)-2-methylpropoxy) carbonyl)-L-leucine (6). TLC system: 10% MeOH in DCM R_{f}: 0.1 LCMS (ESI): m/z 342.34 [M+H]⁺

### Methyl (S)-2-((S)-2-(((2-(3-chlorophenyl)-2-methylpropoxy) carbonyl) amino)-4-methylpentanamido)-3-((S)-2-oxopyrrolidin-3-yl) propanoate (7)

To a stirred solution of ((2-(3-chlorophenyl)-2-methylpropoxy) carbonyl)-L-leucine (6) (10 g, 29.32 mmol) DMF (100 mL) added EDC.HCl (8.401 g, 43.98 mmol), HOBt (5.76 g, 43.98 mmol), DIPEA (15.2 mL, 87.9 mmol) and methyl (S)-2-amino-3-((S)-2-oxopyrrolidin-3-yl)propanoate hydrochloride (amine fragment-2) (6.545 g, 35.19 mmol) at 0° C. simultaneously and stirred at room temperature for 16 h. Reaction mixture was diluted with ice water (100 mL), extracted with ethyl acetate (2×50 mL), dried over sodium sulfate and evaporated under reduced pressure. The crude residue was purified by combi-flash NP, compound eluted at 4% methanol in dichloromethane to afford methyl (S)-2-((S)-2-(((2-(3-chlorophenyl)-2-methylpropoxy)carbonyl)amino)-4-methylpentanamido)-3-((S)-2-oxopyrrolidin-3-yl)propanoate (7). TLC system: 100% Ethyl acetate Rf: 0.4 LCMS (ESI): m/z 510.55 [M+H]⁺

### 2-(3-Chlorophenyl)-2-methylpropyl ((S)-1-(((S)-1-hydroxy-3-((S)-2-oxopyrrolidin-3-yl)propan-2-yl)amino)-4-methyl-1-oxopentan-2-yl)carbamate (8)

To a stirred solution of methyl (S)-2-((S)-2-(((2-(3-chlorophenyl)-2-methylpropoxy)carbonyl) amino)-4-methylpentanamido)-3-((S)-2-oxopyrrolidin-3-yl)propanoate (7) (3 g, 5.893 mmol) in DCM (20 mL) was added 2M LiBH₄ in THF (8.8 mL, 17.681 mmol) at 0° C. and the reaction mixture stirred for 2 h at 0° C. The progress of the reaction was monitored by TLC and LCMS. Reaction mixture was quenched with sat. Ammonium chloride solution (40 mL) and extracted with DCM (2×30 mL). Organic layer was washed with brine solution (30 mL), dried over Na₂SO₄ and concentrated to afford mixture of cpd-8 (2.8 g, 5.81 mmol, 98% yield). The mixture was purified by SFC to afford 2-(3-Chlorophenyl)-2-methylpropyl ((S)-1-(((S)-1-hydroxy-3-((S)-2-oxopyrrolidin-3-yl)propan-2-yl)amino)-4-methyl-1-oxopentan-2-yl)carbamate (8). TLC system: 100% Ethyl acetate Rf: 0.2 LCMS (ESI): m/z 482.50 [M+H]⁺

### 2-(3-Chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C96)

To a stirred solution of 2-methyl-1-phenylpropan-2-yl ((S)-1-(((S)-1-hydroxy-3-((S)-2-oxopyrrolidin-3-yl)propan-2-yl)amino)-4-methyl-1-oxopentan-2-yl)carbamate (8) (1 g, 2.079 mmol) was dissolved in dichloromethane (10 mL) was added Dess-Martin periodinane (2.644 g, 6.237 mmol) at 0° C. and stirred at RT for 2 h. Reaction mixture was diluted with DCM (15 mL) followed by sat. Hypo solution (3×15 mL), followed by sat. NaHCO₃ solution (3×15 mL). Organic layer was dried over anhydrous Na₂SO₄, filtered and concentrated to get crude compound. The crude compound was purified by normal phase silica gel purification 10% MeOH/DCM product was eluted as a 2-(3-chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C96). TLC system: 100% Ethyl acetate Rf: 0.4 LCMS (ESI): m/z 480.2 (M+H)⁺

### Sodium (2S)-2-((S)-2-(((2-(3-chlorophenyl)-2-methylpropoxy)carbonyl)amino)-4-methylpentanamido)-1-hydroxy-3-((S)-2-oxopyrrolidin-3-yl)propane-1-sulfonate (Compound C72)

To a stirred solution of 2-(3-chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C96) (750 mg, 1.565 mmol) was dissolved in Ethyl acetate (2.0 mL), Ethanol (2.0 mL), Water (2.0 mL), and then NaHSO₃ (325 mg, 3.131 mmol) was added and stirred at 50-60° C. for 16 h. The progress of the reaction was monitored by TLC and LCMS. The reaction mixture was filtered through celite pad wash with methanol (10 mL) and filtrate was dried over sodium sulphate, concentrated and the crude compound was washed with combination of DCM/Diethyl ether/Pentane to afford sodium (2S)-2-((S)-2-(((2-(3-chlorophenyl)-2-methylpropoxy)carbonyl)amino)-4-methylpentanamido)-1-hydroxy-3-((S)-2-oxopyrrolidin-3-yl)propane-1-sulfonate (Compound C72). TLC system: 5% Methanol in dichloromethane next 100% Ethylacetate Rf: 0.2 LCMS (ESI): m/z 560.1 (M−Na)⁻

### Example 54: Synthesis of Compound C54 and C69

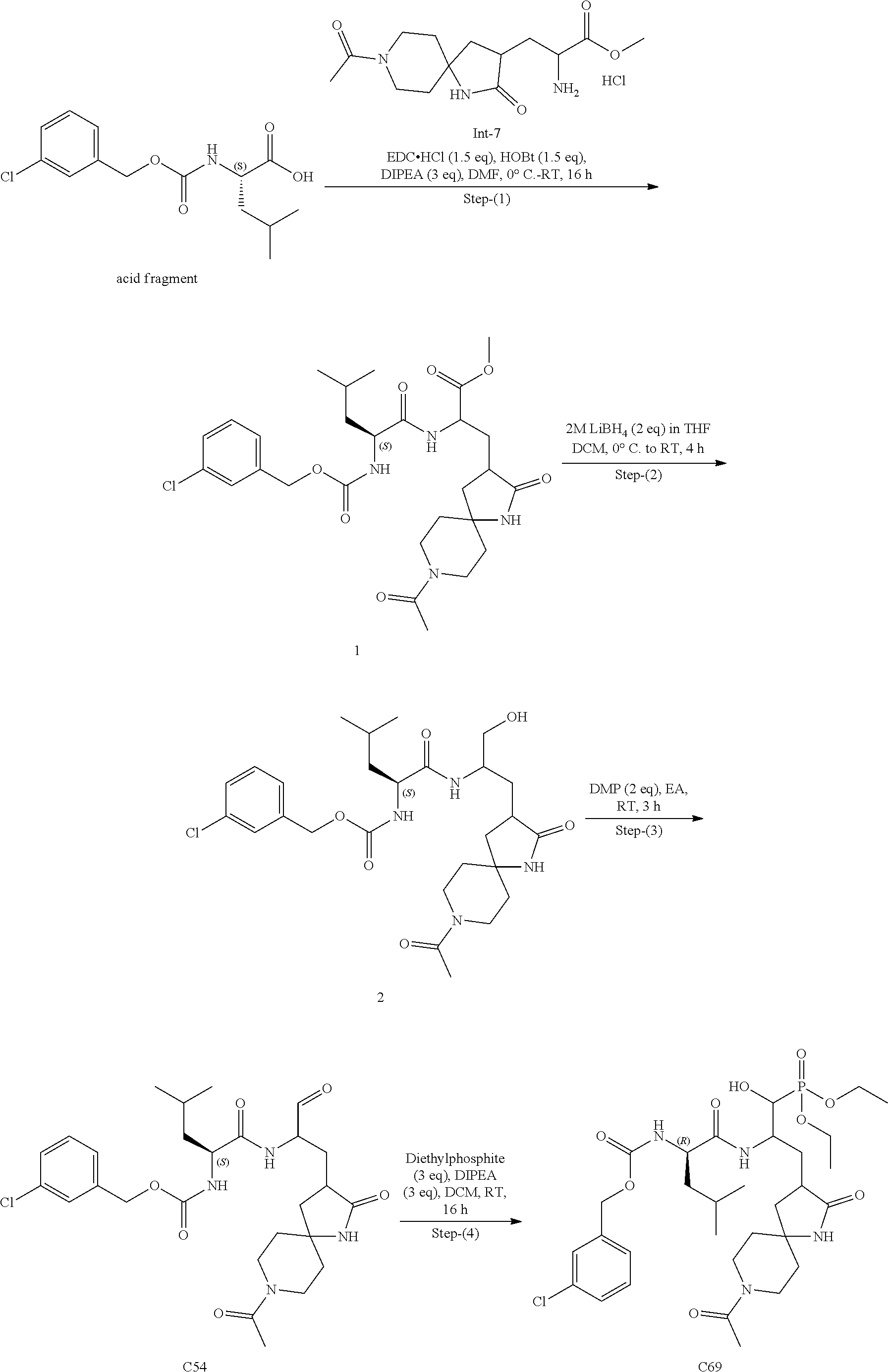

### Methyl 3-(8-acetyl-2-oxo-1,8-diazaspiro[4.5]decan-3-yl)-2-((S)-2-((((3-chlorobenzyl)oxy)carbonyl)amino)-4-methylpentanamido)propanoate (1)

To a stirred solution of (((3-chlorobenzyl)oxy)carbonyl)-L-leucine acid (Acid fragment) (270 mg, 0.903 mmol) in DMF (5 mL) at 0° C. was added EDC.HCl (259 mg, 1.35 mmol), HOBT (183 mg, 1.35 mmol), DIPEA (0.5 mL, 2.7 mmol) and ethyl 3-(8-acetyl-2-oxo-1,8-diazaspiro[4.5]decan-3-yl)-2-aminopropanoate hydrochloride (Int-7) (345 mg, 0.99 mmol) simultaneously and stirred at room temperature for 16 h. The progress of the reaction was monitored by TLC and LCMS. After 16 h, reaction mixture was quenched with ice water (20 mL), extracted with ethyl acetate (2×30 mL), the combined organic layer was dried over sodium sulfate and evaporated under reduced pressure. The crude residue was purified by silica gel column by eluting with 50% ethyl acetate in pet ether to afford methyl 3-(8-acetyl-2-oxo-1,8-diazaspiro[4.5]decan-3-yl)-2-((S)-2-((((3-chlorobenzyl)oxy)carbonyl)amino)-4-methylpentanamido)propanoate (1). TLC system: 5% Methanol in DCM R_{f}: 0.6 LCMS (ESI): m/z 593.59 (M+H)⁺

### 3-Chlorobenzyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2)

To a stirred solution of methyl 3-(8-acetyl-2-oxo-1,8-diazaspiro[4.5]decan-3-yl)-2-((S)-2-((((3-chlorobenzyl)oxy)carbonyl)amino)-4-methylpentanamido)propanoate (1) (280 mg, 0.47 mmol) in DCM (3 mL) was added 2M LiBH₄ in THF (0.4 mL, 0.94 mmol) at 0° C. and the reaction mixture stirred for 2 h at RT. The progress of the reaction was monitored by TLC and LCMS. After 4 h, reaction mixture was quenched with water (20 mL) and extracted with DCM (2×30 mL). Organic layer was washed with brine solution (30 mL), and combined organic layer was dried over Na₂SO₄ and concentrated to afford the 3-chlorobenzyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2). TLC system: 5% MeOH in DCM R_{f} 0.3 LCMS (ESI): m/z 551.38 (M+H)⁺

### 3-Chlorobenzyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C54)

To a stirred solution of 3-chlorobenzyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2) (120 mg, 0.26 mmol) in ethyl acetate (5 mL) was added Dess-Martin periodinane (479 mg, 1.13 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 3-chlorobenzyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C54). TLC system: 5% Methanol in DCM R_{f}: 0.35 LCMS (ESI): m/z 549.50 (M+H)⁺

### 3-Chlorobenzyl ((2R)-1-((3-(8-acetyl-2-oxo-1,8-diazaspiro[4.5]decan-3-yl)-1-(diethoxyphosphoryl)-1-hydroxypropan-2-yl)amino)-4-methyl-1-oxopentan-2-yl)carbamate (Compound C69)

To a stirred solution of 3-chlorobenzyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C54) (200 mg, 0.36 mmol) in DCM (2 mL) was added DIPEA (0.2 mL, 1.09 mmol), diethyl phosphite (0.1 mL, 0.72 mmol) at 0° C. and stirred at RT for 16 h. The progress of the reaction was monitored by TLC and LCMS. The reaction was quenched with water (20 mL) and extracted with DCM (2×30 mL). Organic layer was washed with brine solution (30 mL), and combined organic layer was dried over Na₂SO₄. The Crude was purified by prep HPLC to afford 3-chlorobenzyl ((2R)-1-((3-(8-acetyl-2-oxo-1,8-diazaspiro[4.5]decan-3-yl)-1-(diethoxyphosphoryl)-1-hydroxypropan-2-yl)amino)-4-methyl-1-oxopentan-2-yl)carbamate (Compound C69). TLC system: 5% Methanol in DCM R_{f}: 0.5 LCMS (ESI): m/z 587.2 (M+H)⁺

### Example 55: Synthesis of Compounds C60 and C76

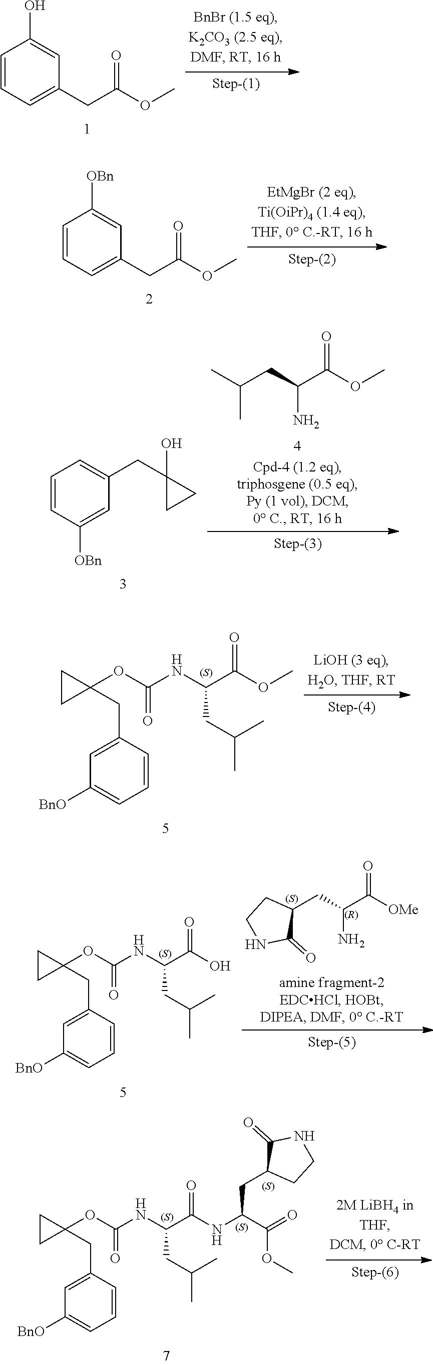

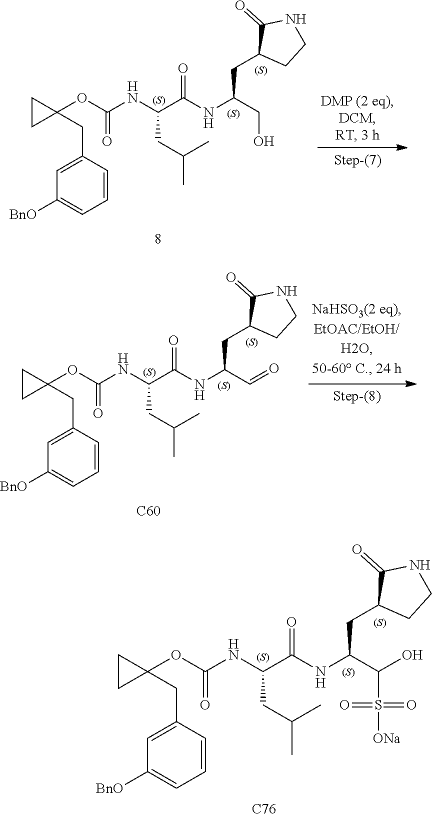

### methyl 2-(3-(benzyloxy)phenyl)acetate (2)

To a stirred solution of methyl 2-(3-hydroxyphenyl) acetate (1) (5 g, 3.012 mmol) in DMF (50 mL) was added K₂CO₃ (12.46 mL, 9.03 mmol) at RT slowly, followed by benzyl bromide (7.7 mL, 4.51 mmol) and the reaction mixture was stirred at RT for 16 h. The progress of the reaction was monitored by TLC. The reaction mixture was quenched with ice cooled water (150 mL) and stirred for 1 h, extracted with diethyl ether (3×100 mL), dried over sodium sulfate and evaporated under reduced pressure. The crude residue was purified by silica gel column by eluting with 7% ethyl acetate in hexane to afford methyl 2-(3-(benzyloxy)phenyl)acetate (2). TLC system: 20% Ethyl acetate in Hexane Rf: 0.5 LCMS (ESI): m/z=257.36 [M+H]⁺

### 1-(3-(benzyloxy)benzyl)cyclopropan-1-ol (3)

To a stirred solution of methyl 2-(3-(benzyloxy)phenyl)acetate (2) (6 g, 23.43 mmol) in THF (60 mL) was added titanium isoperoxide (9.3 mL, 46.61 mmol) at 0° C. slowly, followed by ethyl magnesium bromide (58.5 mL, 58.5 mmol) at 0° C. and the reaction mixture was stirred at RT for 16 h. The progress of the reaction was monitored by TLC. The reaction mixture was quenched with saturated NH₄Cl (50 mL) and filtered through celite pad and washed with ethyl acetate (100 mL), dried over sodium sulfate and evaporated under reduced pressure. The crude residue was purified by silica gel column by eluting with 10% ethyl acetate in hexane to afford 1-benzylcyclopropan-1-ol (3). TLC system: 10% Ethyl acetate in Hexane Rf: 0.3 LCMS (ESI): m/z=255.1 [M+H]⁺

### Methyl ((1-(3-(benzyloxy)benzyl)cyclopropoxy)carbonyl)-L-leucinate (4)

To a stirred solution of 1-benzylcyclopropan-1-ol (3) (2.5 g, 9.82 mmol) in DCM (30 mL) was added pyridine (3 mL) methyl L-leucinate hydrochloride (4) (1.7 g, 11.79 mmol) followed by triphosgene (1.45 g, 4.91 mmol) at 0° C. slowly and stirred at RT for 16 h. The progress of the reaction was monitored by TLC. Reaction mixture was quenched with 1N aq. HCl (50 mL) then extracted with DCM (2×50 mL), washed with 2×50 mL NaHCO₃ solution the organic layer was dried over sodium sulfate and evaporated under reduced pressure. The crude residue was purified by silica gel column by eluting with 15% ethyl acetate in hexane to afford methyl ((1-(3-(benzyloxy)benzyl)cyclopropoxy)carbonyl)-L-leucinate (5). TLC system: 20% Ethyl acetate in hexane Rf: 0.4 LCMS (ESI): m/z=426.47 [M+H]+

### ((1-(3-(Benzyloxy)benzyl)cyclopropoxy)carbonyl)-L-leucine (6)

To a stirred solution of methyl ((1-benzylcyclopropoxy) carbonyl)-leucinate (5) (1.2 g, 2.8 mmol) in THF (10 mL), water (2.5 mL) was added LiOH.H₂O (350 mg, 8.4 mmol) at 0° C. Reaction mixture was stirred at RT for 3 h. The progress of the reaction was monitored by TLC. After consumption of starting material, the reaction mixture was concentrated and acidified with 1N HCl, extracted with ethyl acetate (2×30 mL), dried over sodium sulfate and evaporated under reduced pressure to afford ((1-(3-(benzyloxy)benzyl)cyclopropoxy)carbonyl)-L-leucine (6) which was directly used in the next step. TLC system: 70% Ethyl acetate and pet-ether Rf: 0.2 LCMS (ESI): m/z=412.44 [M+H]⁺

### Methyl (S)-2-((S)-2-(((1-(3-(benzyloxy)benzyl)cyclopropoxy)carbonyl)amino)-4-methylpentanamido)-3-((S)-2-oxopyrrolidin-3-yl)propanoate (6)

To a stirred solution of ((1-(3-(benzyloxy)benzyl)cyclopropoxy)carbonyl)-L-leucine (6) (23.2 g, 7.78 mmol) in DMF (20 mL) was added EDC.HCl (2.2 g, 11.67 mmol), HOBT (1.576 g, 11.67 mmol), DIPEA (4.3 mL, 23.34 mmol) and methyl (R)-2-amino-3-((S)-2-oxopyrrolidin-3-yl)propanoate (amine-fragment-2) (1.73 g, 9.34 mmol) at 0° C. simultaneously and stirred at room temperature for 16 h. Reaction mixture was diluted with ice water (50 mL), extracted with ethyl acetate (2×40 mL), the organic layer was dried over sodium sulfate and evaporated under reduced pressure to afford crude compound. The crude residue was purified by silica gel column by eluting with 50% Ethyl acetate and pet-ether to afford methyl (S)-2-((S)-2-(((1-(3-(benzyloxy) benzyl)cyclopropoxy)carbonyl)amino)-4-methylpentanamido)-3-((S)-2-oxopyrrolidin-3-yl) propanoate (7). TLC system: 50% Ethyl acetate in pet ether Rf: 0.3 LCMS (ESI): m/z=580.78 [M+H]⁺

### 1-(3-(Benzyloxy)benzyl)cyclopropyl ((S)-1-(((S)-1-hydroxy-3-((S)-2-oxopyrrolidin-3-yl)propan-2-yl)amino)-4-methyl-1-oxopentan-2-yl)carbamate (6)

To a stirred solution of methyl (S)-2-((S)-2-(((1-(3-(benzyloxy) benzyl)cyclopropoxy) carbonyl)amino)-4-methylpentanamido)-3-((S)-2-oxopyrrolidin-3-yl) propanoate (7) (1.3 g, 2.24 mmol) in dichloromethane (13 mL) was added 2M LiBH₄ in THF (3.36 mL, 6.6 mmol) at 0° C. and stirred for 2 h. The progress of the reaction was monitored by TLC and LCMS. The reaction mixture was quenched with saturated NH₄Cl solution (50 mL) and extracted with DCM (2×50 mL), the organic layer was dried over sodium sulfate and evaporated under reduced pressure to afford 1-(3-(benzyloxy)benzyl)cyclopropyl ((S)-1-(((S)-1-hydroxy-3-((S)-2-oxopyrrolidin-3-yl) propan-2-yl)amino)-4-methyl-1-oxopentan-2-yl)carbamate (8). TLC system: 70% Ethyl acetate in pet ether Rf: 0.2 LCMS (ESI): m/z=552.46 [M+H]⁺

### 1-(3-(Benzyloxy)benzyl)cyclopropyl ((S)-4-methyl-1-oxo-1-(((S)-1-oxo-3-((S)-2-oxopyrrolidin-3-yl)propan-2-yl)amino)pentan-2-yl)carbamate (Compound C60)

To a stirred solution of 1-benzylcyclopropyl ((S)-1-(((S)-1-hydroxy-3-((S)-2-oxopyrrolidin-3-yl)propan-2-yl)amino)-4-methyl-1-oxopentan-2-yl)carbamate (7) (200 mg, 0.362 mmol) was dissolved in Ethyl acetate (4 mL) was added Dess-Martin periodinane (446 mg, 1.088 mmol) at 0° C. and stirred at RT for 3 h. Reaction mixture was diluted with Ethyl acetate (20 mL) filtered with celite pad filtrate was washed with sat. Hypo solution (3×20 mL), sat. NaHCO₃ solution (3×20 mL). Organic layer was dried over anhydrous Na₂SO₄, filtered and concentrated to afford crude product. The crude product was purified by Prep. HPLC to afford 1-(3-(benzyloxy) benzyl)cyclopropyl ((S)-4-methyl-1-oxo-1-(((S)-1-oxo-3-((S)-2-oxopyrrolidin-3-yl)propan-2-yl) amino)pentan-2-yl)carbamate (Compound C60). TLC system: 10% Methanol in dichloromethane Rf: 0.5 LCMS (ESI): m/z=444.42 (M+H)⁺

### Sodium (2S)-2-((S)-2-(((1-(3-(benzyloxy)benzyl)cyclopropoxy)carbonyl)amino)-4-methylpentanamido)-1-hydroxy-3-((S)-2-oxopyrrolidin-3-yl)propane-1-sulfonate (Compound C76)

To a stirred solution of 1-(3-(benzyloxy) benzyl)cyclopropyl ((S)-4-methyl-1-oxo-1-(((S)-1-oxo-3-((S)-2-oxopyrrolidin-3-yl)propan-2-yl) amino)pentan-2-yl)carbamate (Compound C60) (150 mg, 0.273 mmol) was dissolved in Ethyl acetate (1.0 mL), Ethanol (1.0 mL), Water (1.0 mL), and then NaHSO₃ (31 mg, 0.3 mmol) was added and stirred at 50-60° C. for 24 h. The progress of the reaction was monitored by TLC and LCMS. The reaction mixture was filtered through celite pad wash with methanol (10 mL) and filtrate was dried over sodium sulphate, concentrated and the crude compound was washed with combination of DCM/Diethyl ether/Pentane to afford sodium (2S)-2-((S)-2-(((1-(3-(benzyloxy) benzyl) cyclopropoxy)carbonyl)amino)-4-methylpentanamido)-1-hydroxy-3-((S)-2-oxopyrrolidin-3-yl)propane-1-sulfonate (Compound C76). TLC system: 5% Methanol in dichloromethane next 100% Ethylacetate Rf: 0.2 LCMS (ESI): m/z 630.2 (M−Na)

### Example 56: Synthesis of Compound C64 and C95

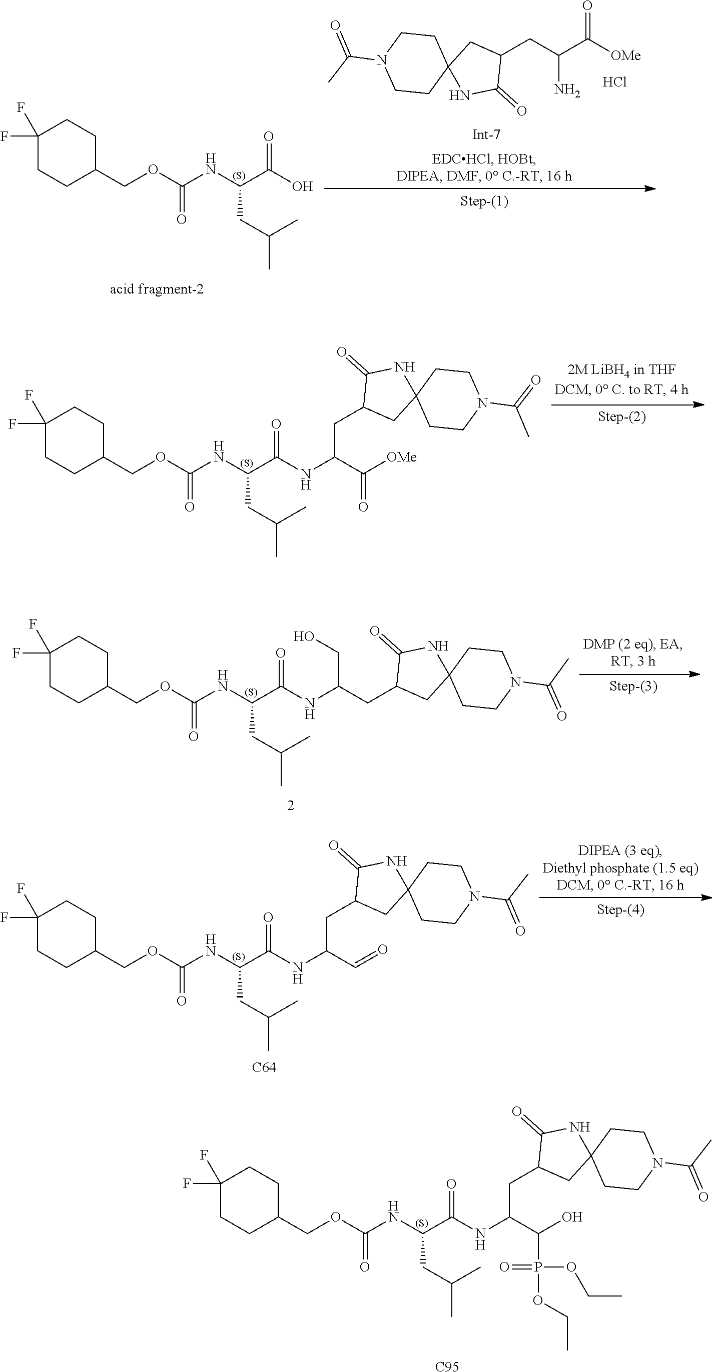

### Methyl 3-(8-acetyl-2-oxo-1,8-diazaspiro[4.5]decan-3-yl)-2-((S)-2-((((4,4-difluorocyclohexyl)methoxy)carbonyl)amino)-4-methylpentanamido)propanoate (1)

To a stirred solution of (((4,4-difluorocyclohexyl)methoxy)carbonyl)-L-leucine (Acid fragment-2) (1.2 g, 3.9 mmol) in DMF (20 mL) at 0° C. was added EDC.HCl (1.1 g, 5.86 mmol), HOBT (0.79 g, 5.86 mmol), DIPEA (2.1 mL, 11.72 mmol) and methyl 3-(8-acetyl-2-oxo-1,8-diazaspiro[4.5]decan-3-yl)-2-aminopropanoate hydrochloride (Int-7) (1.49 g, 4.29 mmol) simultaneously and stirred at room temperature for 16 h. The progress of the reaction was monitored by TLC and LCMS. After 16 h, reaction mixture was quenched with ice water (20 mL), extracted with ethyl acetate (2×100 mL), the combined organic layer was dried over sodium sulfate and evaporated under reduced pressure. The crude residue was purified by silica gel column by eluting with 50% ethyl acetate in pet ether to afford methyl 3-(8-acetyl-2-oxo-1,8-diazaspiro[4.5]decan-3-yl)-2-((S)-2-((((4,4difluorocyclohexyl)methoxy)carbonyl) amino)-4-methylpentanamido)propanoate (1). TLC system: 5% Methanol in DCM R_{f}: 0.65 LCMS (ESI): m/z 601.71 (M+H)⁺

### (4,4-Difluorocyclohexyl)methyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2)

To a stirred solution of methyl 3-(8-acetyl-2-oxo-1,8-diazaspiro[4.5]decan-3-yl)-2-((S)-2-((((4,4difluorocyclohexyl)methoxy)carbonyl) amino)-4-methylpentanamido)propanoate (1) (500 mg, 0.85 mmol) in DCM (3 mL) was added 2M LiBH₄ in THF (0.85 mL, 1.7 mmol) at 0° C. and the reaction mixture stirred for 2 h at RT. The progress of the reaction was monitored by TLC and LCMS. After 4 h, reaction mixture was quenched with water (20 mL) and extracted with DCM (2×30 mL). Organic layer was washed with brine solution (30 mL), and combined organic layer was dried over Na₂SO₄ and concentrated to afford (4,4-difluorocyclohexyl)methyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2). TLC system: 5% MeOH in DCM R_{f} 0.3 LCMS (ESI): m/z 559.68 (M+H)⁺

### (4,4-Difluorocyclohexyl)methyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C64)

To a stirred solution of (4,4-difluorocyclohexyl)methyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2) (400 mg, 0.71 mmol) in ethyl acetate (4 mL) was added Dess-Martin periodinane (1.2 g, 2.87 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford (4,4-difluorocyclohexyl)methyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C64). TLC system: 5% Methanol in DCM R_{f}: 0.3 LCMS (ESI): m/z 557.3 (M+H)⁺

### (4,4-Difluorocyclohexyl)methyl ((2S)-1-((3-(8-acetyl-2-oxo-1,8-diazaspiro[4.5]decan-3-yl)-1-(diethoxyphosphoryl)-1-hydroxypropan-2-yl)amino)-4-methyl-1-oxopentan-2-yl)carbamate (Compound C95)

To a stirred solution of (4,4-difluorocyclohexyl)methyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C64) (100 mg, 0.179 mmol) was dissolved in DCM (2 mL) was added DIPEA (0.1 mL, 0.53 mmol) and diethyl phosphite (0.08 mL, 0.53 mmol) at 0° C. and stirred at RT for 16 h. Reaction mixture was quenched with ice water (10 mL) extracted with DCM (3×20 mL). Organic layer was dried over anhydrous Na₂SO₄, filtered and purified by prep HPLC to afford (4,4-difluorocyclohexyl)methyl ((2S)-1-((3-(8-acetyl-2-oxo-1,8-diazaspiro[4.5]decan-3-yl)-1-(diethoxyphosphoryl)-1-hydroxypropan-2-yl)amino)-4-methyl-1-oxopentan-2-yl)carbamate (Compound C95). TLC system: 5% Methanol in DCM R_{f}: 0.3 LCMS (ESI): m/z 695.3.3 (M+H)⁺

### Example 57: Synthesis of Compounds C66 and C85

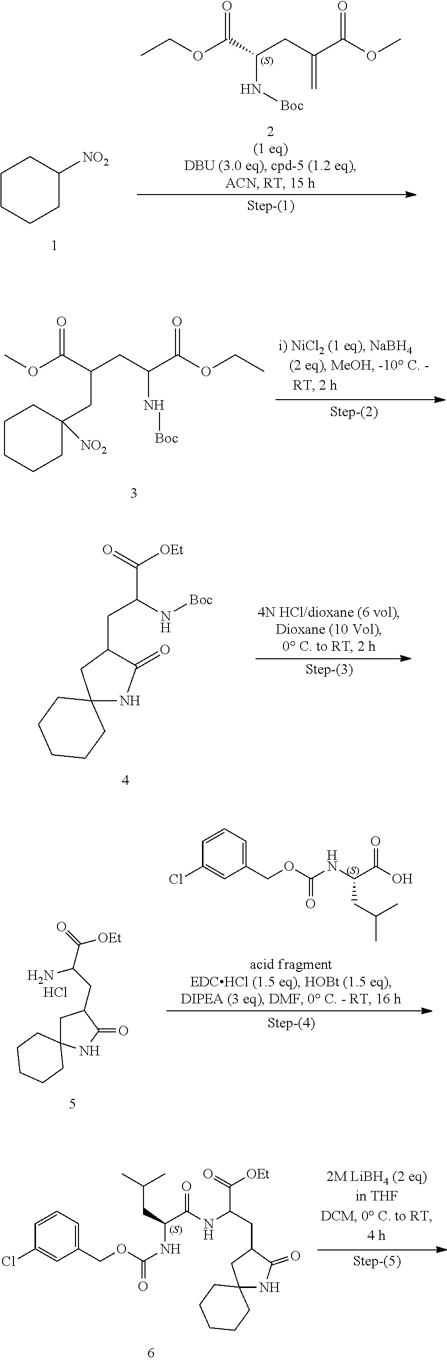

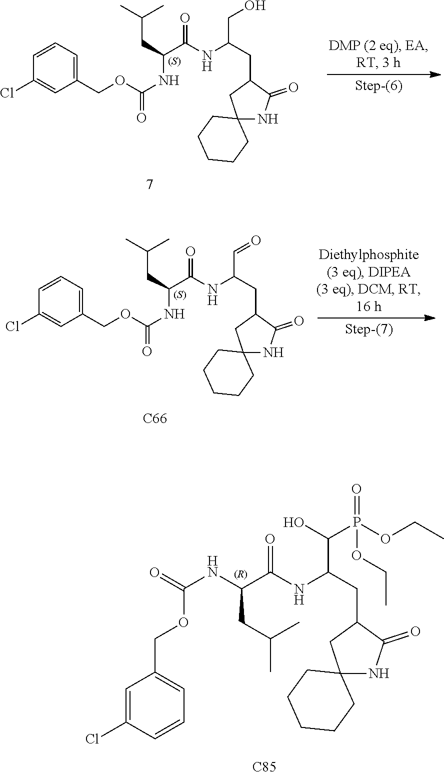

### 1-Ethyl 5-methyl 2-((tert-butoxycarbonyl)amino)-4-((1-nitrocyclohexyl)methyl)pentanedioate

To a stirred solution of 1-ethyl 5-methyl (S)-2-((tert-butoxycarbonyl)amino)-4-methylenepentanedioate (2) (1.86 g, 6.17 mmol) in ACN (8 mL) was added nitro cyclohexane (1) (800 mg, 6.17 mmol) and DBU (1.8 mL, 12.4 mmol) at 0° C. and stirred at RT for 16 h. The progress of the reaction was monitored by TLC and LCMS. Reaction mixture was evaporated under reduced pressure. The crude residue was purified by normal phase chromatography to afford 1-ethyl 5-methyl 2-((tert-butoxycarbonyl)amino)-4-((1-nitrocyclohexyl)methyl)pentanedioate (3). TLC system: 60% Ethyl acetate/Pete there R_{f}: 0.6 LCMS (ESI): m/z 453.48 (M+Na+H)⁺

### Ethyl 2-((tert-butoxycarbonyl)amino)-3-(2-oxo-1-azaspiro[4.5]decan-3-yl)propanoate (4)

To a stirred solution of 1-ethyl 5-methyl 2-((tert-butoxycarbonyl)amino)-4-((1-nitrocyclohexyl)methyl)pentanedioate (3) (500 mg, 1.16 mmol) in methanol (5 mL) was added nickel chloride (151 mg, 1.16 mmol), followed by sodium borohydride (221 mg, 5.81 mmol) at −10° C. and stirred at RT for 2 h. The progress of the reaction was monitored by TLC and LCMS. The reaction mixture was quenched with water (5 mL) and extracted with 10% MeOH/DCM (3×25 mL), combined organic layers were washed with brine solution (10 mL), dried over sodium sulfate and evaporated under reduced pressure. The crude residue was purified by normal phase chromatography to afford ethyl 2-((tert-butoxycarbonyl)amino)-3-(2-oxo-1-azaspiro[4.5]decan-3-yl)propanoate (4). TLC system: 10% MeOH/DCM R_{f}: 0.3 LCMS (ESI): m/z 369.55 (M+H)⁺

### Ethyl 2-amino-3-(2-oxo-1-azaspiro[4.5]decan-3-yl)propanoate hydrochloride (5)

To a stirred solution of ethyl 2-((tert-butoxycarbonyl)amino)-3-(2-oxo-1-azaspiro[4.5]decan-3-yl)propanoate (4) (2 g, 5.43 mmol) in DCM (10 mL) was added 1,4-dioxane.HCl (20 mL) at 0° C. and stirred at RT for 2 h. The progress of the reaction was monitored by TLC. Reaction mixture was evaporated under reduced pressure. The crude residue was trituration with n-pentane to afford ethyl 2-amino-3-(2-oxo-1-azaspiro[4.5]decan-3-yl)propanoate hydrochloride (5). TLC system: 15% MeOH/DCM R_{f}: 0.1 LCMS (ESI): m/z 269.03 (M+H)⁺

### Ethyl 2-((S)-2-((((3-chlorobenzyl)oxy)carbonyl)amino)-4-methylpentanamido)-3-(2-oxo-1-azaspiro[4.5]decan-3-yl)propanoate (6)

To a stirred solution of (S)-2-((((3-chlorobenzyl)oxy)carbonyl)amino)-3-cyclohexylpropanoic acid (Acid fragment) (1.2 g, 4.01 mmol) in DMF (15 mL) at 0° C. was added EDC.HCl (1.1 g, 6.01 mmol), HOBT (800 mg, 6.01 mmol), DIPEA (2 mL, 12 mmol) and ethyl 2-amino-3-(2-oxo-1-azaspiro[4.5]decan-3-yl)propanoate hydrochloride (1.4 g, 4.8 mmol) simultaneously and stirred at room temperature for 16 h. The progress of the reaction was monitored by TLC and LCMS. After 16 h, reaction mixture was quenched with ice water (50 mL), extracted with ethyl acetate (2×50 mL), the combined organic layer was dried over sodium sulfate and evaporated under reduced pressure to get ethyl 2-((S)-2-((((3-chlorobenzyl)oxy)carbonyl)amino)-4-methylpentanamido)-3-(2-oxo-1-azaspiro[4.5]decan-3-yl)propanoate (6). TLC system: 5% Methanol in DCM R_{f}: 0.6 LCMS (ESI): m/z 550.61 (M+H)⁺

### Ethyl 2-((S)-2-((((3-chlorobenzyl)oxy)carbonyl)amino)-4-methylpentanamido)-3-(2-oxo-1-azaspiro[4.5]decan-3-yl)propanoate (7)

To a stirred solution of ethyl 2-((S)-2-((((3-chlorobenzyl)oxy)carbonyl)amino)-4-methylpentanamido)-3-(2-oxo-1-azaspiro[4.5]decan-3-yl)propanoate (6) (800 mg, 1.45 mmol) in DCM (10 mL) was added 2M LiBH₄ in THF (1.45 mL, 2.91 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), the combined organic layer was dried over sodium sulfate and evaporated under reduced pressure to afford ethyl 2-((S)-2-((((3-chlorobenzyl)oxy)carbonyl)amino)-4-methylpentanamido)-3-(2-oxo-1-azaspiro[4.5]decan-3-yl)propanoate (7). TLC system: 5% MeOH in DCM R_{f} 0.2 LCMS (ESI): m/z 508.20 (M+H)⁺

### 3-Chlorobenzyl ((2S)-4-methyl-1-oxo-1-((1-oxo-3-(2-oxo-1-azaspiro[4.5]decan-3-yl)propan-2-yl)amino)pentan-2-yl)carbamate (Compound C66)

To a stirred solution of 3-chlorobenzyl ((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)carbamate (7) (100 mg, 0.19 mmol) in ethyl acetate (5 mL) was added Dess-Martin periodinane (334 mg, 0.78 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 3-chlorobenzyl ((2S)-4-methyl-1-oxo-1-((1-oxo-3-(2-oxo-1-azaspiro[4.5]decan-3-yl)propan-2-yl)amino)pentan-2-yl)carbamate (Compound C66). TLC system: 5% Methanol in DCM R_{f}: 0.4 LCMS (ESI): m/z 506.2 (M+H)⁺

### 3-Chlorobenzyl ((2R)-1-((1-(diethoxyphosphoryl)-1-hydroxy-3-(2-oxo-1-azaspiro[4.5]decan-3-yl)propan-2-yl)amino)-4-methyl-1-oxopentan-2-yl)carbamate (Compound C85)

To a stirred solution of 3-chlorobenzyl ((2S)-4-methyl-1-oxo-1-((1-oxo-3-(2-oxo-1-azaspiro[4.5]decan-3-yl)propan-2-yl)amino)pentan-2-yl)carbamate (Compound C66) (200 mg, 0.39 mmol) in DCM (3 mL) was added DIPEA (0.2 mL, 1.1 mmol), diethyl phosphite (0.14 mL, 1.1 mmol) at 0° C. and stirred at RT for 16 h. The progress of the reaction was monitored by TLC and LCMS. Quenched with water (20 mL) and extracted with DCM (2×30 mL). Organic layer was washed with brine solution (30 mL), and combined organic layer was dried over Na₂SO₄. Crude was purified by prep HPLC to afford 3-chlorobenzyl ((2R)-1-((1-(diethoxyphosphoryl)-1-hydroxy-3-(2-oxo-1-azaspiro[4.5]decan-3-yl)propan-2-yl)amino)-4-methyl-1-oxopentan-2-yl)carbamate (Compound C85). TLC system: 5% Methanol in DCM R_{f}: 0.5 LCMS (ESI): m/z 644.2 (M+H)⁺

### Example 58: Synthesis of Compounds C67 and C82

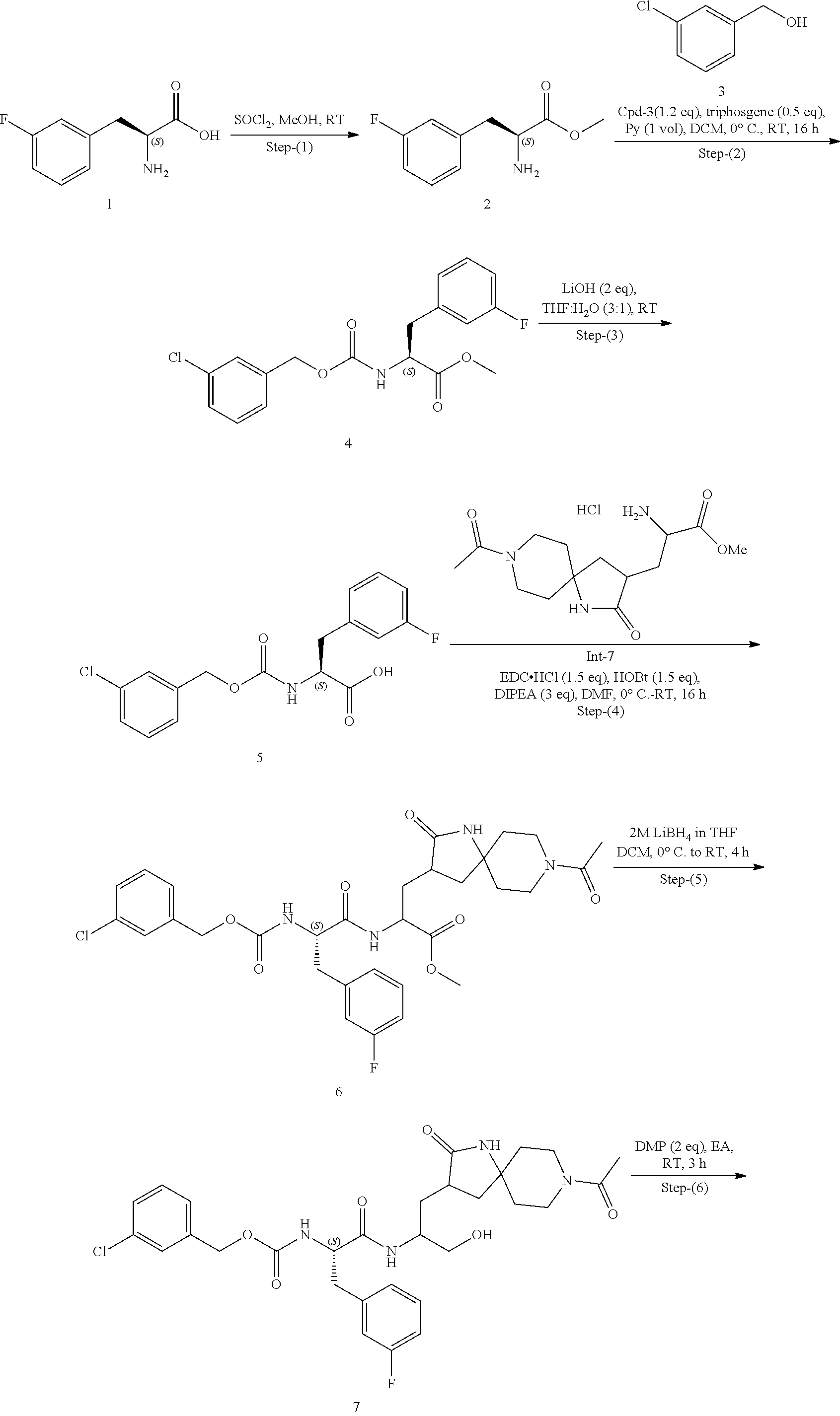

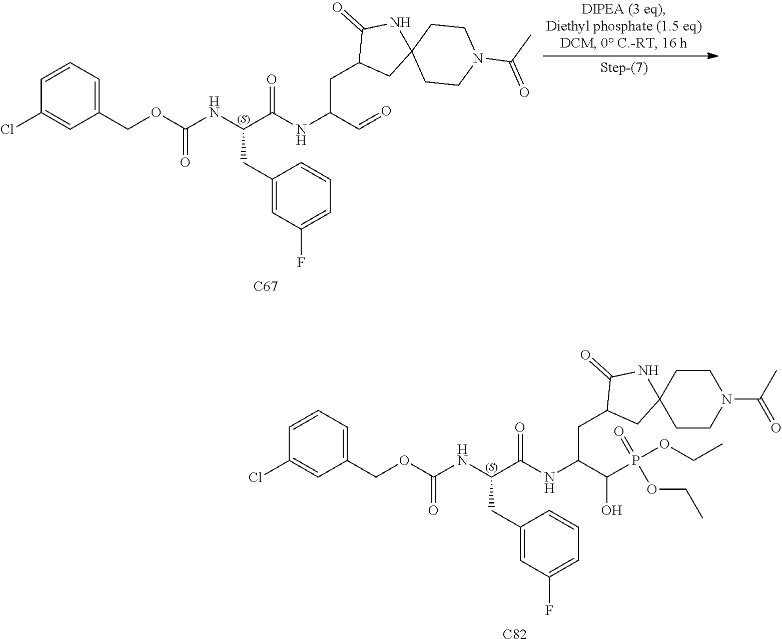

### Methyl (S)-2-amino-3-(3-fluorophenyl)propanoate (2)

To a stirred solution of (S)-2-amino-3-(3-fluorophenyl)propanoic acid (1) (500 mg, 2.73 mmol) in methanol (10 mL) was added acetyl chloride (1 mL, 10 vol) and cat. DMF simultaneously at 0° C. and the reaction mixture stirred for 16 h at 80° C. The progress of the reaction was monitored by TLC and LCMS. Reaction mass evaporated to dryness and triturated with n-pentane afford methyl (S)-2-amino-3-(3-fluorophenyl)propanoate (2). TLC system: 5% MeOH in DCM R_{f} 0.3 LCMS (ESI): m/z 198.24 (M+H)⁺

### Methyl (S)-2-((((3-chlorobenzyl)oxy)carbonyl)amino)-3-(3-fluorophenyl)propanoate (4)

To a stirred solution of methyl (S)-2-amino-3-(3-fluorophenyl)propanoate (3) (108 mg, 0.76 mmol), methyl (S)-2-amino-3-(3-fluorophenyl)propanoate (2) (100 mg, 0.507 mmol) in DCM (40 mL) was added pyridine (0.1 mL, 1 vol) followed by triphosgene (225 mg, 0.76 mmol) at 0° C. and stirred at room temperature for 16 h. The progress of the reaction was monitored by TLC and LCMS. Reaction mixture was quenched with ice water (30 mL), extracted with DCM (2×30 mL). The combined organic layer was dried over sodium sulfate, filtered and evaporated under reduced pressure. The crude residue was purified by combi-flash, compound eluted at 8% ethyl acetate in pet ether to afford methyl (S)-2-((((3-chlorobenzyl)oxy)carbonyl)amino)-3-(3-fluorophenyl)propanoate (4). TLC system: 40% Ethyl acetate in Pet ether R_{f}: 0.5 LCMS (ESI): m/z 366.30 [M+H]⁺

### (S)-2-((((3-chlorobenzyl)oxy)carbonyl)amino)-3-(3-fluorophenyl)propanoic acid (5)

To a stirred solution of methyl (S)-2-((((3-chlorobenzyl)oxy)carbonyl)amino)-3-(3-fluorophenyl)propanoate (4) methyl (1.2 g, 3.28 mmol) in THF (8 mL), water (2 mL) was added lithium hydroxide (404 mg, 9.86 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×40 mL), dried over sodium sulfate, concentrated under reduced pressure to afford (S)-2-((((3-chlorobenzyl)oxy)carbonyl)amino)-3-(3-fluorophenyl)propanoic acid (5). TLC system: 10% MeOH in DCM Rf: 0.2 LCMS (ESI): m/z 351.92 (M+H)⁺

### Methyl 3-(8-acetyl-2-oxo-1,8-diazaspiro[4.5]decan-3-yl)-2-((S)-2-((((3-chlorobenzyl)oxy)carbonyl)amino)-3-(3-fluorophenyl)propanamido)propanoate (6)

To a stirred solution of (R)-2-((((3-chlorobenzyl)oxy)carbonyl)amino)-3-(3-fluorophenyl)propanoic acid (5) (970 mg, 2.76 mmol) in DMF (15 mL) at 0° C. was added EDC.HCl (790 mg, 790 mmol), HOBT (595 mg, 4.41 mmol), DIPEA (1.4 mL, 8.28 mmol) and methyl 3-(8-acetyl-2-oxo-1,8-diazaspiro[4.5]decan-3-yl)-2-aminopropanoate hydrochloride (Int-7) (1.15 g, 3.31 mmol) simultaneously and stirred at room temperature for 16 h. The progress of the reaction was monitored by TLC and LCMS. After 16 h, reaction mixture was quenched with ice water (50 mL), extracted with ethyl acetate (2×100 mL), the combined organic layer was dried over sodium sulfate and evaporated under reduced pressure. The crude residue was purified by silica gel column by eluting with 60% ethyl acetate in pet ether to afford methyl 3-(8-acetyl-2-oxo-1,8-diazaspiro[4.5]decan-3-yl)-2-((S)-2-((((3-chlorobenzyl)oxy)carbonyl)amino)-3-(3-fluorophenyl) propanamido)propanoate (6). TLC system: 5% Methanol in DCM R_{f}: 0.65 LCMS (ESI): m/z 631.31 (M+H)⁺

### 3-Chlorobenzyl ((2S)-1-((1-(8-acetyl-2-oxo-1,8-diazaspiro[4.5]decan-3-yl)-3-hydroxypropan-2-yl)amino)-3-(3-fluorophenyl)-1-oxopropan-2-yl)carbamate (7)

To a stirred solution of methyl 3-(8-acetyl-2-oxo-1,8-diazaspiro[4.5]decan-3-yl)-2-((S)-2-((((3-chlorobenzyl)oxy)carbonyl)amino)-3-(3-fluorophenyl) propanamido)propanoate (6) (600 mg, 0.95 mmol) in DCM (3 mL) was added 2M LiBH₄ in THF (0.95 mL, 1.9 mmol) at 0° C. and the reaction mixture stirred for 2 h at RT. The progress of the reaction was monitored by TLC and LCMS. After 4 h, reaction mixture was quenched with water (20 mL) and extracted with DCM (2×30 mL). Organic layer was washed with brine solution (30 mL), and combined organic layer was dried over Na₂SO₄ and concentrated to afford 3-chlorobenzyl ((2S)-1-((1-(8-acetyl-2-oxo-1,8-diazaspiro[4.5]decan-3-yl)-3-hydroxypropan-2-yl)amino)-3-(3-fluorophenyl)-1-oxopropan-2-yl)carbamate (7). TLC system: 5% MeOH in DCM R_{f} 0.2 LCMS (ESI): m/z 603.7 (M+H)⁺

### 3-Chlorobenzyl ((2S)-1-((1-(8-acetyl-2-oxo-1,8-diazaspiro[4.5]decan-3-yl)-3-oxopropan-2-yl)amino)-3-(3-fluorophenyl)-1-oxopropan-2-yl)carbamate (Compound C67)

To a stirred solution of 3-chlorobenzyl ((2S)-1-((1-(8-acetyl-2-oxo-1,8-diazaspiro[4.5]decan-3-yl)-3-hydroxypropan-2-yl)amino)-3-(3-fluorophenyl)-1-oxopropan-2-yl)carbamate (7) (500 mg, 0.79 mmol) in ethyl acetate (4 mL) was added Dess-Martin periodinane (1 g, 2.38 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 3-chlorobenzyl ((2S)-1-((1-(8-acetyl-2-oxo-1,8-diazaspiro[4.5]decan-3-yl)-3-oxopropan-2-yl)amino)-3-(3-fluorophenyl)-1-oxopropan-2 yl)carbamate (Compound C67). TLC system: 5% Methanol in DCM R_{f}: 0.4 LCMS (ESI): m/z 601.2 (M+H)⁺

### 3-Chlorobenzyl ((2S)-1-((3-(8-acetyl-2-oxo-1,8-diazaspiro[4.5]decan-3-yl)-1-(diethoxyphosphoryl)-1-hydroxypropan-2-yl)amino)-3-(3-fluorophenyl)-1-oxopropan-2-yl)carbamate (Compound C67)

To a stirred solution of 3-chlorobenzyl ((2S)-1-((1-(8-acetyl-2-oxo-1,8-diazaspiro[4.5]decan-3-yl)-3-oxopropan-2-yl)amino)-3-(3-fluorophenyl)-1-oxopropan-2-yl)carbamate (Compound C82) (100 mg, 0.18 mmol) was dissolved in DCM (2 mL) was added DIPEA (0.1 mL, 0.54 mmol) and diethyl phosphite (0.07 mL, 0.54 mmol) at 0° C. and stirred at RT for 16 h. Reaction mixture was quenched with ice water (10 mL) extracted with DCM (3×20 mL). Organic layer was dried over anhydrous Na₂SO₄, filtered and purified by prep HPLC to afford pure 3-chlorobenzyl ((2S)-1-((3-(8-acetyl-2-oxo-1,8-diazaspiro[4.5]decan-3-yl)-1-(diethoxyphosphoryl)-1-hydroxypropan-2-yl)amino)-3-(3-fluorophenyl)-1-oxopropan-2-yl)carbamate (Compound C67). TLC system: 100% EtOAc in pet ether Rf: 0.25 LCMS (ESI): m/z 739.2 (M+H)⁺

### Example 59: Synthesis of Compound C68

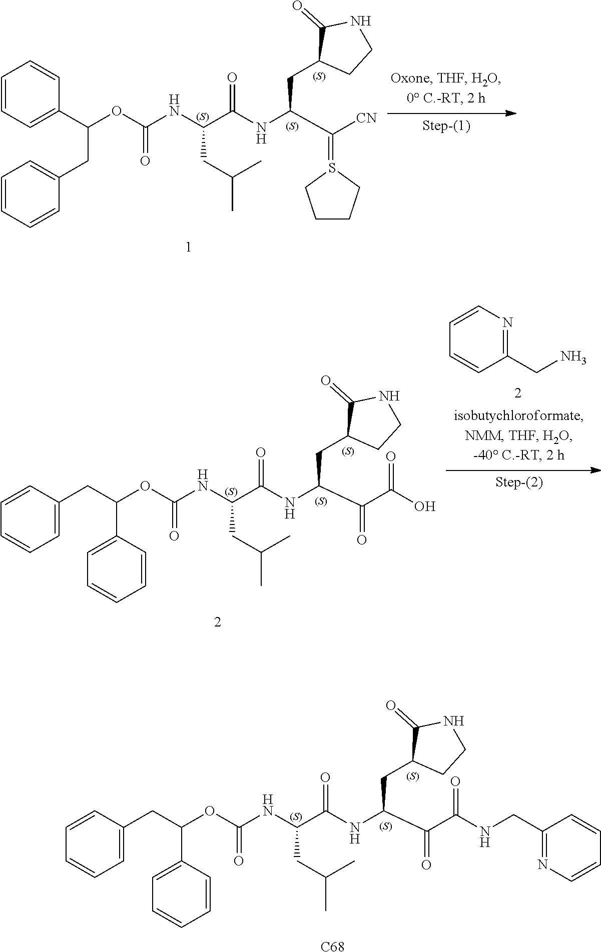

### (3S)-3-((2S)-2-(((1,2-Diphenylethoxy)carbonyl)amino)-4-methylpentanamido)-2-oxo-4-((S)-2-oxopyrrolidin-3-yl)butanoic acid (2)

To a stirred solution of 1,2-diphenylethyl ((S)-1-(((S)-1-cyano-3-((S)-2-oxopyrrolidin-3-yl)-1-(tetrahydro-1λ⁴-thiophen-1-ylidene)propan-2-yl)amino)-4-methyl-1-oxopentan-2-yl)carba-mate (1) (750 mg, 1.2 mmol) in THF/H₂O (5 mL/2.5 mL) at 0° C. was added oxone (745 mg, 2.4 mmol) and the mixture was stirred at RT for 2 h. Then a solution of 2-picolylamine (0.06 mL, 0.4189 mmol) in THF/DMF (0.5 mL/0.25 mL) was added. Then to the mixture was added 1N HCl and adjusted the PH to acidic and extracted with ethyl acetate. Organic layer was washed with brine solution (30 mL), dried over Na₂SO₄ and concentrated to afford (3S)-3-((2S)-2-(((1,2-diphenylethoxy)carbonyl)amino)-4-methylpentanamido)-2-oxo-4-((S)-2-oxopyrrolidin-3-yl)butanoic acid (2). TLC system: 15% MeOH in DCM Rf: 0.1 LCMS (ESI): m/z 538.44 [M+H]⁺

### 1,2-Diphenylethyl ((S)-1-(((S)-3,4-dioxo-1-((S)-2-oxopyrrolidin-3-yl)-4-((pyridin-2-ylmethyl)amino)butan-2-yl)amino)-4-methyl-1-oxopentan-2-yl)carbamate (Compound C68)

To a stirred solution of (3S)-3-((2S)-2-(((1,2-diphenylethoxy)carbonyl)amino)-4-methylpentanamido)-2-oxo-4-((S)-2-oxopyrrolidin-3-yl)butanoic acid (2) (150 mg, 0.2793 mmol) in THF (3 mL) at −40° C. was added isobutyl chloroformate (0.045 mL, 0.33516 mmol) and NMM (0.045 mL, 0.4189 mmol) drop-wise. The mixture was stirred at −40° C. for 30 min. Then a solution of 2-picolylamine (0.06 mL, 0.4189 mmol) in THF/DMF (0.5 mL/0.25 mL) was added. The mixture was stirred at −40° C. for 2 h. Reaction mixture was quenched with sat. NaHCO₃ solution (10 mL) and extracted with ethyl acetate (2×20 mL). Organic layer was washed with brine solution (30 mL), dried over Na₂SO₄ and concentrated to get crude compound. The crude compound was purified by prep HPLC to afford 1,2-Diphenylethyl ((S)-1-(((S)-3,4-dioxo-1-((S)-2-oxopyrrolidin-3-yl)-4-((pyridin-2-ylmethyl)amino)butan-2-yl)amino)-4-methyl-1-oxopentan-2-yl)carbamate (Compound C68). TLC system: 10% MeOH in DCM Rf: 0.45 LCMS (ESI): m/z 628.3 [M+H]⁺

### Example 60: Synthesis of Compounds C70 and C84

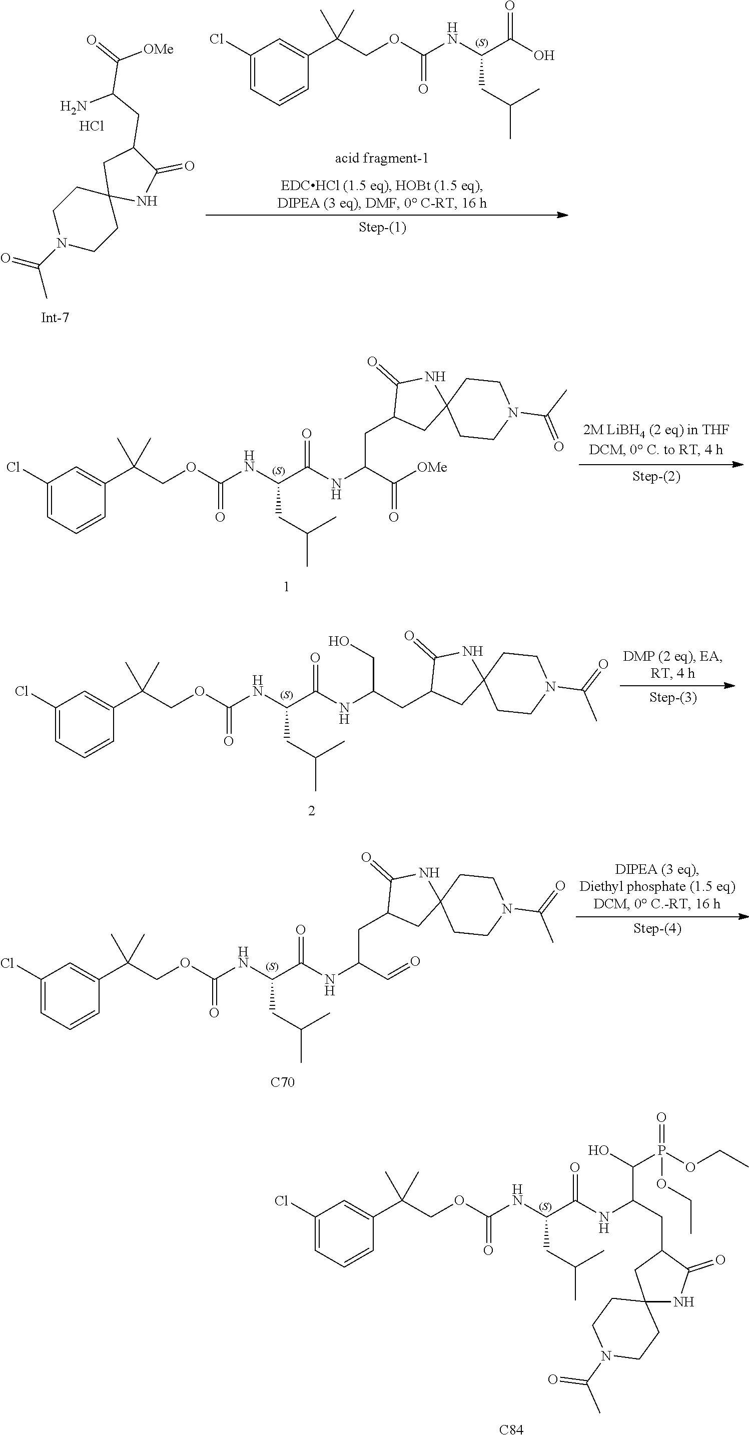

### Methyl 3-(8-acetyl-2-oxo-1,8-diazaspiro[4.5]decan-3-yl)-2-((S)-3-cyclohexyl-2-(1H-indole-2-carboxamido)propanamido)propanoate (1)

To a stirred solution of ((2-(3-chlorophenyl)-2-methylpropoxy)carbonyl)-L-leucine (Acid fragment-1) (1 g, 2.9 mmol) in DMF (20 mL) at 0° C. was added EDC.HCl (1 g, 5.2 mmol), HOBT (0.7 g, 5.2 mmol), DIPEA (1.5 mL, 8.7 mmol) and methyl 3-(8-acetyl-2-oxo-1,8-diazaspiro[4.5]decan-3-yl)-2-aminopropanoate hydrochloride (Int-7) (1.2 g, 3.5 mmol) simultaneously and stirred at room temperature for 16 h. The progress of the reaction was monitored by TLC and LCMS. After 16 h, reaction mixture was quenched with ice water (20 mL), extracted with ethyl acetate (2×100 mL), the combined organic layer was dried over sodium sulfate and evaporated under reduced pressure. The crude residue was purified by silica gel column by eluting with 50% ethyl acetate in pet ether to afford methyl 3-(8-acetyl-2-oxo-1,8-diazaspiro[4.5]decan-3-yl)-2-((S)-3-cyclohexyl-2-(1H-indole-2-carboxamido)propanamido)propanoate (1) TLC system: 5% Methanol in DCM R_{f}: 0.65 LCMS (ESI): m/z 521.27 (M+H)⁺

### 2-(3-Chlorophenyl)-2-methylpropyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2)

To a stirred solution of methyl 3-(8-acetyl-2-oxo-1,8-diazaspiro[4.5]decan-3-yl)-2-((S)-3-cyclohexyl-2-(1H-indole-2-carboxamido)propanamido)propanoate (1) (350 mg, 0.56 mmol) in DCM (3 mL) was added 2M LiBH₄ in THF (0.56 mL, 1.1 mmol) at 0° C. and the reaction mixture stirred for 2 h at RT. The progress of the reaction was monitored by TLC and LCMS. After 4 h, reaction mixture was quenched with water (20 mL) and extracted with DCM (2×30 mL). Organic layer was washed with brine solution (30 mL), and combined organic layer was dried over Na₂SO₄ and concentrated to afford 2-(3-chlorophenyl)-2-methylpropyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2). TLC system: 5% MeOH in DCM R_{f} 0.25 LCMS (ESI): m/z 593.26 (M+H)⁺

### 2-(3-Chlorophenyl)-2-methylpropyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C70)

To a stirred solution 2-(3-chlorophenyl)-2-methylpropyl ((2S)-1-((1-(8-acetyl-2-oxo-1,8-diazaspiro[4.5]decan-3-yl)-3-hydroxypropan-2-yl)amino)-4-methyl-1-oxopentan-2-yl)carbamate (2) (240 mg, 0.4 mmol) in ethyl acetate (4 mL) was added Dess-Martin periodinane (515 mg, 1.21 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 2-(3-chlorophenyl)-2-methylpropyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C70). TLC system: 5% Methanol in DCM R_{f}: 0.3 LCMS (ESI): m/z 591.3 (M+H)⁺

### 2-(3-Chlorophenyl)-2-methylpropyl ((2S)-1-((3-(8-acetyl-2-oxo-1,8-diazaspiro[4.5]decan-3-yl)-1-(diethoxyphosphoryl)-1-hydroxypropan-2-yl)amino)-4-methyl-1-oxopentan-2-yl)carbamate (Compound C84)

To a stirred solution of 2-(3-chlorophenyl)-2-methylpropyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C70) (270 mg, 0.28 mmol) was dissolved in DCM (2 mL) was added DIPEA (0.24 mL, 0.86 mmol) and diethyl phosphite (0.25 mL, 0.21 mmol) at 0° C. and stirred at RT for 16 h. Reaction mixture was quenched with ice water (10 mL) extracted with DCM (3×20 mL). Organic layer was dried over anhydrous Na₂SO₄, filtered and purified by prep HPLC to afford 2-(3-chlorophenyl)-2-methylpropyl ((2S)-1-((3-(8-acetyl-2-oxo-1,8-diazaspiro[4.5]decan-3-yl)-1-(diethoxyphosphoryl)-1-hydroxypropan-2-yl)amino)-4-methyl-1-oxopentan-2-yl)carbamate (Compound C84). TLC system: 80% EtOAc in pet ether Rf: 0.2 LCMS (ESI): m/z 729.2 (M+H)⁺

### Example 61: Synthesis of Compound C73

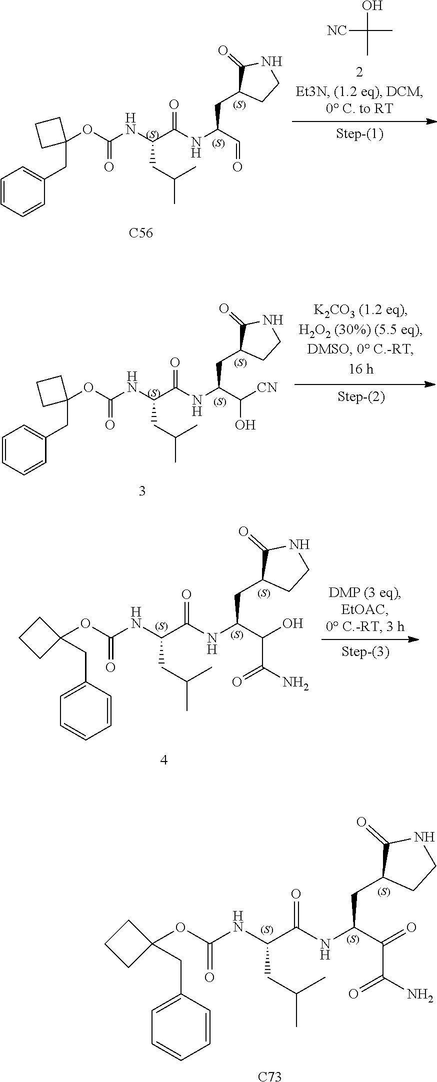

### 1-Benzylcyclobutyl ((2S)-1-(((2S)-1-cyano-1-hydroxy-3-((S)-2-oxopyrrolidin-3-yl)propan-2-yl)amino)-4-methyl-1-oxopentan-2-yl)carbamate (3)

To a stirred solution of 1-benzylcyclobutyl ((S)-4-methyl-1-oxo-1-(((S)-1-oxo-3-((S)-2-oxopyrrolidin-3-yl)propan-2-yl)amino)pentan-2-yl)carbamate (Compound C56) (700 mg, 1.552 mmolin DCM (10 mL), was added Et₃N (0.27 mL, 1.862 mmol) followed by addition of acetone cyanohydrin (2) (263 mg, 3.104 mmol) at 0° C. and reaction mixture allowed to stirred at room temperature for 16 h. The progress of the reaction was monitored by TLC and LCMS. Reaction mixture was diluted with water (50 mL), and extracted with dichloromethane (2×30 mL), dried over sodium sulfate, concentrated under reduced pressure to afford 1-benzylcyclobutyl ((2S)-1-(((2S)-1-cyano-1-hydroxy-3-((S)-2-oxopyrrolidin-3-yl)propan-2-yl)amino)-4-methyl-1-oxopentan-2-yl)carbamate (3). TLC system: 10% MeOH in DCM Rf: 0.3 LCMS (ESI): m/z 485.75 [M+H]⁺

### 1-Benzylcyclobutyl ((2S)-1-(((2S)-4-amino-3-hydroxy-4-oxo-1-((S)-2-oxopyrrolidin-3-yl)butan-2-yl)amino)-4-methyl-1-oxopentan-2-yl)carbamate (4)

To a stirred solution of 1-benzylcyclobutyl ((2S)-1-(((2S)-1-cyano-1-hydroxy-3-((S)-2-oxopyrrolidin-3-yl)propan-2-yl)amino)-4-methyl-1-oxopentan-2-yl)carbamate (3) (500 mg, 1.033 mmol) DMSO (10 mL) was added K₂CO₃ (213 mg, 1.549 mmol) and H₂O₂ (30%) (0.6 mL, 5.681 mmol) at 0° C. and stirred at room temperature for 3 h. Reaction mixture was diluted with water (50 mL), extracted with dichloromethane (2×20 mL), dried over sodium sulfate and evaporated under reduced pressure to afford 1-benzylcyclobutyl((2S)-1-(((2S)-4-amino-3-hydroxy-4-oxo-1-((S)-2-oxopyrrolidin-3-yl)butan-2-yl)amino)-4-methyl-1-oxopentan-2-yl)carbamate (4). TLC system: 10% Methanol in dichloromethane Rf: 0.4 LCMS (ESI): m/z 503.23 [M+H]⁺

### 1-Benzylcyclobutyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C73)

To a stirred solution of 1-benzylcyclobutyl ((2S)-1-(((2S)-4-amino-3-hydroxy-4-oxo-1-((S)-2-oxopyrrolidin-3-yl)butan-2-yl)amino)-4-methyl-1-oxopentan-2-yl)carbamate (4) (450 mg, 0.896 mmol) in EtOAc (10 mL) was added Dess-Martin periodinane (1.3 g, 3.286 mmol) at 0° C. and stirred at RT for 3 h. The progress of the reaction was monitored by TLC and LCMS. Reaction mixture was diluted with DCM (50 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 1-benzylcyclobutyl ((S)-1-(((S)-4-amino-3,4-dioxo-1-((S)-2-oxopyrrolidin-3-yl) butan-2-yl)amino)-4-methyl-1-oxopentan-2-yl)carbamate (Compound C73). TLC system: 5% Methanol in DCM R_{f}: 0.5 LCMS (ESI): m/z 501.2 [M+H]⁺

### Example 62: Synthesis of Compounds C74 and C94

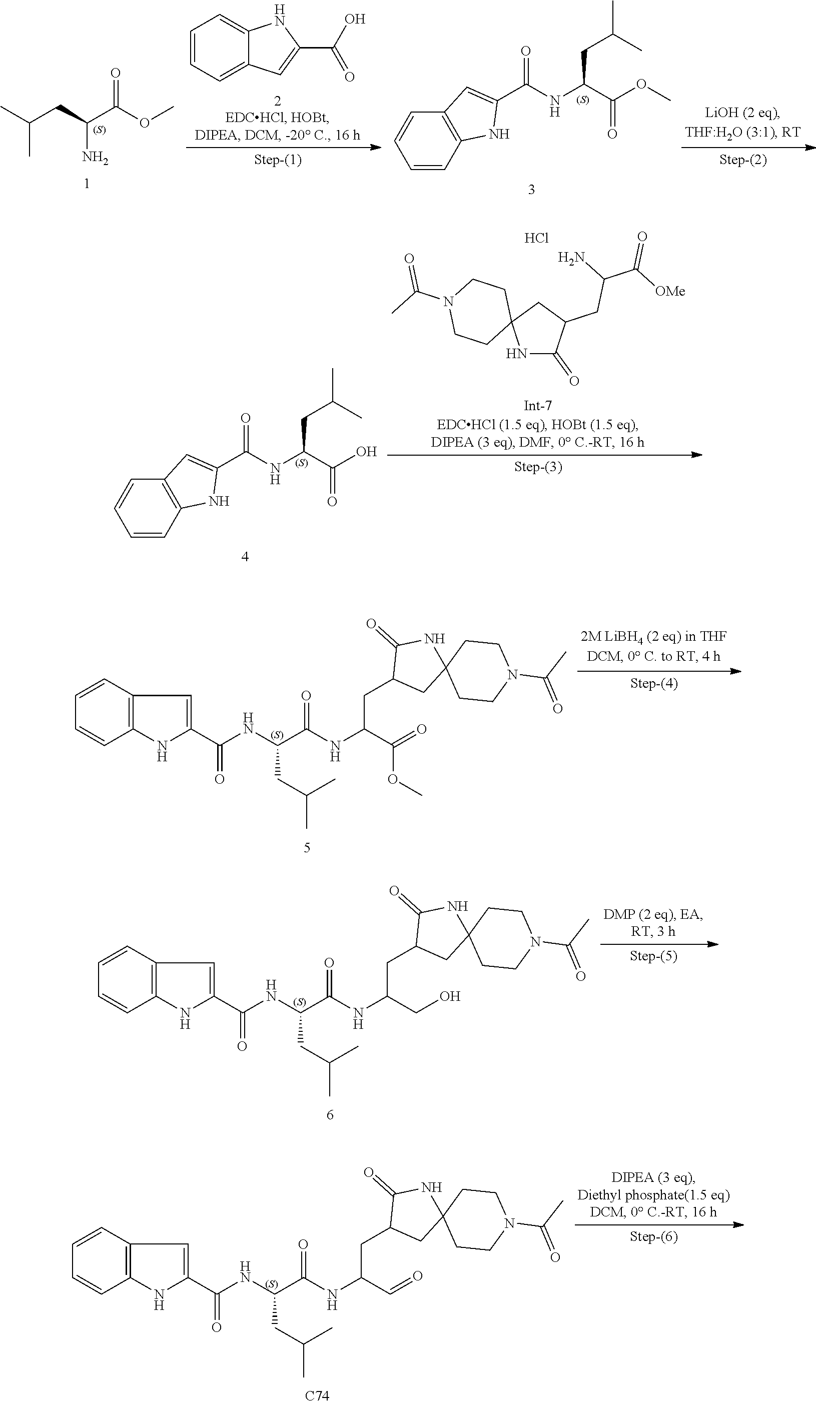

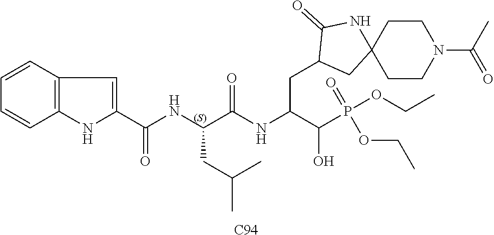

### Methyl (1H-indole-2-carbonyl)-L-leucinate (3)

To a stirred solution of 1H-indole-2-carboxylic acid (2) (1 g, 6.21 mmol) in DMF (20 mL) at 0° C. was added EDC.HCl (1.7 g, 9.31 mmol), HOBT (1.2 g, 5.47 mmol), triethylamine (2.6 mL, 4.5 mmol) and methyl L-leucinate (2) 1.2 g, 1.5 mmol) simultaneously and stirred at room temperature for 16 h. The progress of the reaction was monitored by TLC and LCMS. After 16 h, reaction mixture was quenched with ice water (50 mL), extracted with ethyl acetate (2×100 mL), the combined organic layer was dried over sodium sulfate and evaporated under reduced pressure. The crude residue was purified by silica gel column by eluting with 50% ethyl acetate in pet ether to afford methyl (1H-indole-2-carbonyl)-L-leucinate (3). TLC system: 5% Methanol in DCM R_{f}: 0.65 LCMS (ESI): m/z 289.28 (M+H)⁺

### (1H-indole-2-carbonyl)-L-leucine (4)

To a stirred solution of methyl (1H-indole-2-carbonyl)-L-leucinatemethyl (3) (0.8 g, 2.77 mmol) in THF (4 mL), water (1 mL) was added lithium hydroxide (349 mg, 8.33 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×40 mL), dried over sodium sulfate, concentrated under reduced pressure to afford (1H-indole-2-carbonyl)-L-leucine (4). TLC system: 10% MeOH in DCM Rf: 0.2 LCMS (ESI): m/z 275.1 (M+H)⁺

### Methyl 2-((S)-2-(1H-indole-2-carboxamido)-4-methylpentanamido)-3-(8-acetyl-2-oxo-1,8-diazaspiro[4.5]decan-3-yl)propanoate (5)

To a stirred solution of (1H-indole-2-carbonyl)-D-leucine (4) (1 g, 3.649 mmol) in DMF (15 mL) at 0° C. was added EDC.HCl (1.03 g, 5.47 mmol), HOBT (0.32 g, 5.47 mmol), DIPEA (1.9 mL, 10 mmol) and methyl 3-(8-acetyl-2-oxo-1,8-diazaspiro[4.5]decan-3-yl)-2-aminopropanoate hydrochloride (Int-7) (1.39 g, 4 mmol) simultaneously and stirred at room temperature for 16 h. The progress of the reaction was monitored by TLC and LCMS. After 16 h, reaction mixture was quenched with ice water (50 mL), extracted with ethyl acetate (2×100 mL), the combined organic layer was dried over sodium sulfate and evaporated under reduced pressure. The crude residue was purified by silica gel column by eluting with 50% ethyl acetate in pet ether to afford methyl 2-((S)-2-(1H-indole-2-carboxamido)-4-methylpentanamido)-3-(8-acetyl-2-oxo-1,8-diazaspiro[4.5]decan-3-yl)propanoate (5). TLC system: 5% Methanol in DCM R_{f}: 0.65 LCMS (ESI): m/z 554.76 (M+H)⁺

### N-((2S)-1-((1-(8-acetyl-2-oxo-1,8-diazaspiro[4.5]decan-3-yl)-3-hydroxypropan-2-yl)amino)-4-methyl-1-oxopentan-2-yl)-1H-indole-2-carboxamide (6)

To a stirred solution of methyl 2-((S)-2-(1H-indole-2-carboxamido)-4-methylpentanamido)-3-(8-acetyl-2-oxo-1,8-diazaspiro[4.5]decan-3-yl)propanoate (5) (900 mg, 1.62 mmol) in DCM (3 mL) was added 2M LiBH₄ in THF (1.6 mL, 3.25 mmol) at 0° C. and the reaction mixture stirred for 2 h at RT. The progress of the reaction was monitored by TLC and LCMS. After 4 h, reaction mixture was quenched with water (20 mL) and extracted with DCM (2×30 mL). Organic layer was washed with brine solution (30 mL), and combined organic layer was dried over Na₂SO₄ and concentrated to afford N-((2S)-1-((1-(8-acetyl-2-oxo-1,8-diazaspiro[4.5]decan-3-yl)-3-hydroxypropan-2-yl)amino)-4-methyl-1-oxopentan-2-yl)-1H-indole-2-carboxamide (6). TLC system: 5% MeOH in DCM R_{f} 0.2 LCMS (ESI): m/z 526.75 (M+H)⁺

### N-((2S)-1-((1-(8-acetyl-2-oxo-1,8-diazaspiro[4.5]decan-3-yl)-3-oxopropan-2-yl)amino)-4-methyl-1-oxopentan-2-yl)-1H-indole-2-carboxamide (Compound C74)

To a stirred solution of N-((2S)-1-((1-(8-acetyl-2-oxo-1,8-diazaspiro[4.5]decan-3-yl)-3-hydroxypropan-2-yl)amino)-4-methyl-1-oxopentan-2-yl)-1H-indole-2-carboxamide (6) (200 mg, 0.38 mmol) in ethyl acetate (4 mL) was added Dess-Martin periodinane (0.48 g, 0.38 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford N-((2S)-1-((1-(8-acetyl-2-oxo-1,8-diazaspiro[4.5]decan-3-yl)-3-oxopropan-2-yl)amino)-4-methyl-1-oxopentan-2-yl)-1H-indole-2-carboxamide (Compound C74). TLC system: 5% Methanol in DCM R_{f}: 0.3 LCMS (ESI): m/z 524.2 (M+H)⁺

### Diethyl (2-((S)-2-(1H-indole-2-carboxamido)-4-methylpentanamido)-3-(8-acetyl-2-oxo-1,8-diazaspiro[4.5]decan-3-yl)-1-hydroxypropyl)phosphonate (Compound C94)

To a stirred solution of N-((2S)-1-((1-(8-acetyl-2-oxo-1,8-diazaspiro[4.5]decan-3-yl)-3-oxopropan-2-yl)amino)-4-methyl-1-oxopentan-2-yl)-1H-indole-2-carboxamide (Compound C74) (30 mg, 0.05 mmol) was dissolved in DCM (2 mL) was added DIPEA (0.03 mL, 0.15 mmol) and diethyl phosphite (0.03 mL, 0.15 mmol) at 0° C. and stirred at RT for 16 h. Reaction mixture was quenched with ice water (10 mL) extracted with DCM (3×20 mL). Organic layer was dried over anhydrous Na₂SO₄, filtered and purified by prep HPLC to afford pure diethyl (3-(8-acetyl-2-oxo-1,8-diazaspiro[4.5]decan-3-yl)-2-((S)-3-cyclohexyl-2-(1H-indole-2-carboxamido)propanamido)-1-hydroxypropyl)phosphonate (Compound C94). TLC system: 100% EtOAc in pet ether Rf: 0.3 LCMS (ESI): m/z 662.2 (M+H)⁺

### Example 63: Synthesis of Compounds C75 and C81

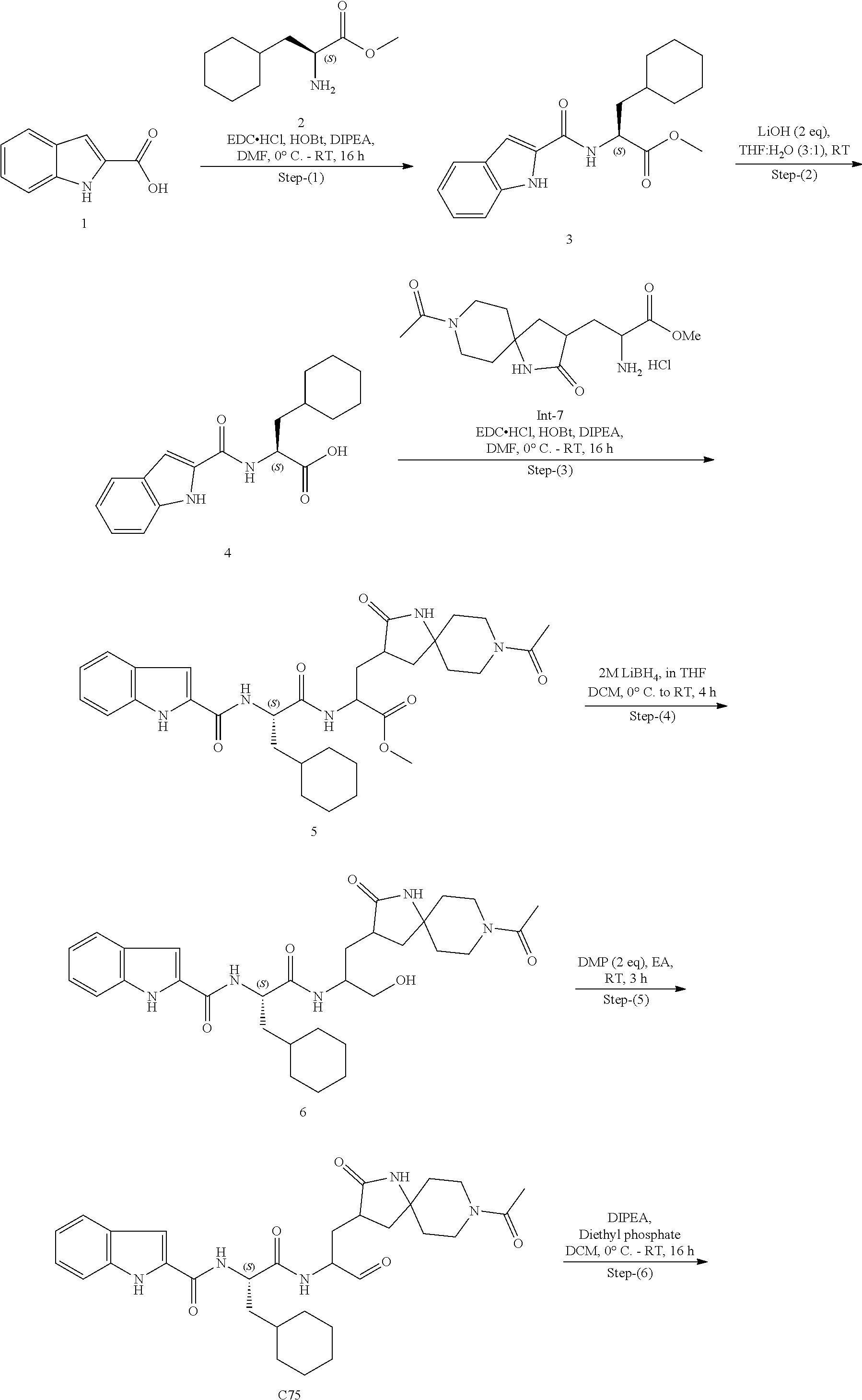

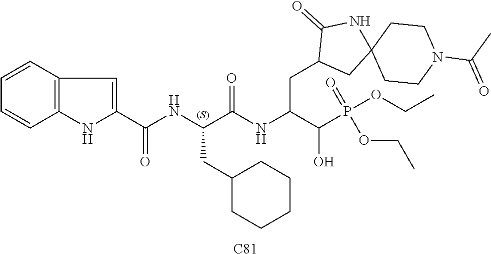

### Methyl (S)-3-cyclohexyl-2-(1H-indole-2-carboxamido)propanoate (3)

To a stirred solution of 1H-indole-2-carboxylic acid (1) (2 g, 12.42 mmol) in DMF (20 mL) at 0° C. was added EDC.HCl (3.5 g, 18.6 mmol), HOBT (1.5 g, 12.4 mmol), triethylamine (5.2 mL, 37.2 mmol) and methyl (S)-2-amino-3-cyclohexylpropanoate (2) (3.4 g, 18.6 mmol) simultaneously and stirred at room temperature for 16 h. The progress of the reaction was monitored by TLC and LCMS. After 16 h, reaction mixture was quenched with ice water (50 mL), extracted with ethyl acetate (2×100 mL), the combined organic layer was dried over sodium sulfate and evaporated under reduced pressure. The crude residue was purified by silica gel column by eluting with 50% ethyl acetate in pet ether to afford methyl (S)-3-cyclohexyl-2-(1H-indole-2-carboxamido)propanoate (3). TLC system: 5% Methanol in DCM R_{f}: 0.65 LCMS (ESI): m/z 329.33 (M+H)⁺

### (S)-3-cyclohexyl-2-(1H-indole-2-carboxamido)propanoic acid (4)

To a stirred solution of methyl (S)-3-cyclohexyl-2-(1H-indole-2-carboxamido)propanoate (3) (7 g, 24.3 mmol) in THF (12 mL), water (3 mL) was added lithium hydroxide (3 g, 72.9 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×100 mL), dried over sodium sulfate, concentrated under reduced pressure to afford (S)-3-cyclohexyl-2-(1H-indole-2-carboxamido)propanoic acid (4). TLC system: 10% MeOH in DCM Rf: 0.2 LCMS (ESI): m/z 315.1 (M+H)⁺

### Methyl 3-(8-acetyl-2-oxo-1,8-diazaspiro[4.5]decan-3-yl)-2-((S)-3-cyclohexyl-2-(1H-indole-2-carboxamido)propanamido)propanoate (5)

To a stirred solution of (R)-3-cyclohexyl-2-(1H-indole-2-carboxamido)propanoic acid (Acid fragment-5) (1 g, 3.18 mmol) in DMF (20 mL) at 0° C. was added EDC.HCl (0.88 g, 4.6 mmol), HOBT (0.62 g, 4.6 mmol), DIPEA (1.6 mL, 9.3 mmol) and methyl 3-(8-acetyl-2-oxo-1,8-diazaspiro[4.5]decan-3-yl)-2-aminopropanoate hydrochloride (Int-7) (1.3 g, 3.8 mmol) simultaneously and stirred at room temperature for 16 h. The progress of the reaction was monitored by TLC and LCMS. After 16 h, reaction mixture was quenched with ice water (20 mL), extracted with ethyl acetate (2×100 mL), the combined organic layer was dried over sodium sulfate and evaporated under reduced pressure. The crude residue was purified by silica gel column by eluting with 50% ethyl acetate in pet ether to afford methyl 3-(8-acetyl-2-oxo-1,8-diazaspiro[4.5]decan-3-yl)-2-((S)-3-cyclohexyl-2-(1H-indole-2-carboxamido)propanamido)propanoate (5). TLC system: 5% Methanol in DCM R_{f}: 0.65 LCMS (ESI): m/z 594.28 (M+H)⁺

### N-((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)-1H-indole-2-carboxamide (6)

To a stirred solution of methyl 3-(8-acetyl-2-oxo-1,8-diazaspiro[4.5]decan-3-yl)-2-((S)-3-cyclohexyl-2-(1H-indole-2-carboxamido)propanamido)propanoate (5) (600 mg, 1.08 mmol) in DCM (3 mL) was added 2M LiBH₄ in THF (1 mL, 2.1 mmol) at 0° C. and the reaction mixture stirred for 2 h at RT. The progress of the reaction was monitored by TLC and LCMS. After 4 h, reaction mixture was quenched with water (20 mL) and extracted with DCM (2×30 mL). Organic layer was washed with brine solution (30 mL), and combined organic layer was dried over Na₂SO₄ and concentrated to afford N-((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)-1H-indole-2-carboxamide (6). TLC system: 5% MeOH in DCM R_{f} 0.2 LCMS (ESI): m/z 566.3 (M+H)⁺

### N-((2S)-1-((1-(8-acetyl-2-oxo-1,8-diazaspiro[4.5]decan-3-yl)-3-oxopropan-2-yl)amino)-3-cyclohexyl-1-oxopropan-2-yl)-1H-indole-2-carboxamide (Compound C75)

To a stirred solution of N-((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)-1H-indole-2-carboxamide (6) (500 mg, 0.88 mmol) in ethyl acetate (4 mL) was added Dess-Martin periodinane (1.12 g, 2.87 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford N-((2S)-1-((1-(8-acetyl-2-oxo-1,8-diazaspiro[4.5]decan-3-yl)-3-oxopropan-2-yl)amino)-3-cyclohexyl-1-oxopropan-2-yl)-1H-indole-2-carboxamide (Compound C75). TLC system: 5% Methanol in DCM R_{f}: 0.3 LCMS (ESI): m/z 564.2 (M+H)⁺

### Diethyl (3-(8-acetyl-2-oxo-1,8-diazaspiro[4.5]decan-3-yl)-2-((S)-3-cyclohexyl-2-(1H-indole-2-carboxamido)propanamido)-1-hydroxypropyl)phosphonate (Compound C81)

To a stirred solution of N-((2S)-1-((1-(8-acetyl-2-oxo-1,8-diazaspiro[4.5]decan-3-yl)-3-oxopropan-2-yl)amino)-3-cyclohexyl-1-oxopropan-2-yl)-1H-indole-2-carboxamide (Compound C75) (30 mg, 0.05 mmol) was dissolved in DCM (2 mL) was added DIPEA (0.03 mL, 0.15 mmol) and diethyl phosphite (0.03 mL, 0.15 mmol) at 0° C. and stirred at RT for 16 h. Reaction mixture was quenched with ice water (10 mL) extracted with DCM (3×20 mL). Organic layer was dried over anhydrous Na₂SO₄, filtered and purified by prep HPLC to afford diethyl (3-(8-acetyl-2-oxo-1,8-diazaspiro[4.5]decan-3-yl)-2-((S)-3-cyclohexyl-2-(1H-indole-2-carboxamido)propanamido)-1-hydroxypropyl)phosphonate (Compound C81). TLC system: 80% EtOAc in pet ether Rf: 0.3 LCMS (ESI): m/z 702.3 (M+H)⁺

### Example 64: Synthesis of Compounds C78 and C99

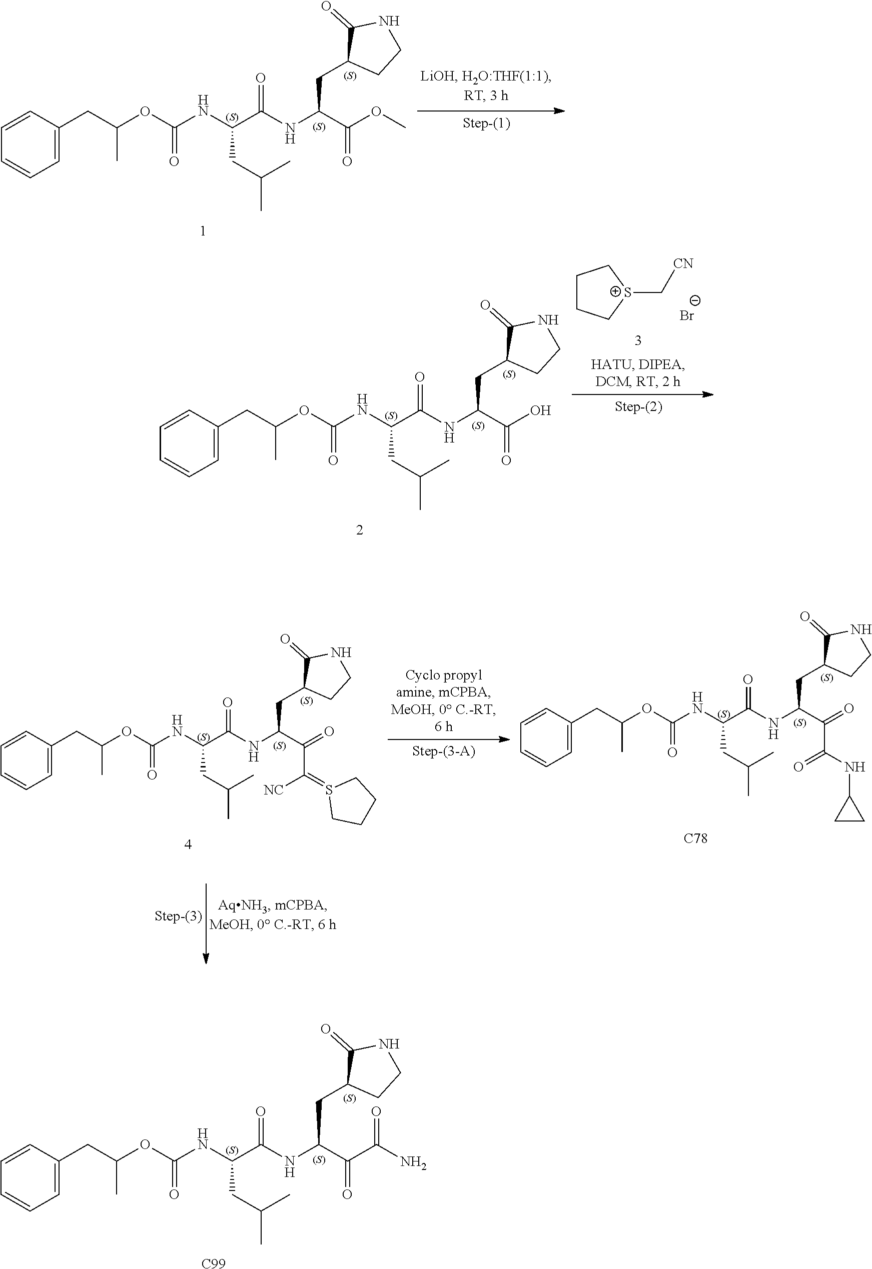

### (2S)-2-((2S)-4-methyl-2-((((1-phenylpropan-2-yl)oxy)carbonyl)amino)pentanamido)-3-((S)-2-oxopyrrolidin-3-yl)propanoic acid (2)

To a stirred solution of methyl (2S)-2-((2S)-4-methyl-2-((((1-phenylpropan-2-yl)oxy)carbonyl)amino)pentanamido)-3-((S)-2-oxopyrrolidin-3-yl)propanoate (1) (1.5 g, 3.253 mmol) in THF (10 mL), water (8 mL) was added lithium hydroxide (234 mg, 9.7613 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-((2S)-4-methyl-2-((((1-phenylpropan-2-yl)oxy)carbonyl)amino)pentanamido)-3-((S)-2-oxopyrrolidin-3-yl)propanoic acid (2). TLC system: 10% MeOH in DCM Rf: 0.2 LCMS (ESI): m/z 448.66 [M+H]⁺

### 1-phenylpropan-2-yl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4)

To a stirred solution of (2S)-2-((2S)-4-methyl-2-((((1-phenylpropan-2-yl)oxy)carbonyl)amino)pentanamido)-3-((S)-2-oxopyrrolidin-3-yl)propanoic acid (2) (1.1 g, 2.46 mmol) DCM (15 mL) added HATU (1.4 g, 3.691 mmol), DIPEA (1.36 mL, 7.382 mmol) and 1-(cyanomethyl)tetrahydro-1H-thiophen-1-iumbromide (3) (608 mg, 2.953 mmol) at 0° C. simultaneously and stirred at room temperature for 2 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 1-phenylpropan-2-yl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4). TLC system: 10% Methanol in dichloromethane Rf: 0.4 LCMS (ESI): m/z 557.89 [M+H]⁺

### 1-phenylpropan-2-yl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C99)

To a stirred solution of 1-phenylpropan-2-yl((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (150 mg, 0.269 mmol) in methanol (5 mL) was added mCPBA (31 mg, 0.179 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford 1-phenylpropan-2-yl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C99). TLC system: 10% Methanol in dichloromethane Rf: 0.2 LCMS (ESI): m/z 475.2 [M+H]⁺

### 1-Phenylpropan-2-yl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C78)

To a stirred solution of 1-phenylpropan-2-yl((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (200 mg, 0.359 mmol) in methanol (5 mL) was added mCPBA (154 mg, 0.899 mmol) at 0° C. and the reaction mixture stirred for 2 h at 0° C. and added cyclopropanamine (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep-HPLC to afford 1-phenylpropan-2-yl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C78). TLC system: 10% Methanol in dichloromethane Rf: 0.3 LCMS (ESI): m/z 515.2 [M+H]⁺

### Example 65: Synthesis of Compound C79

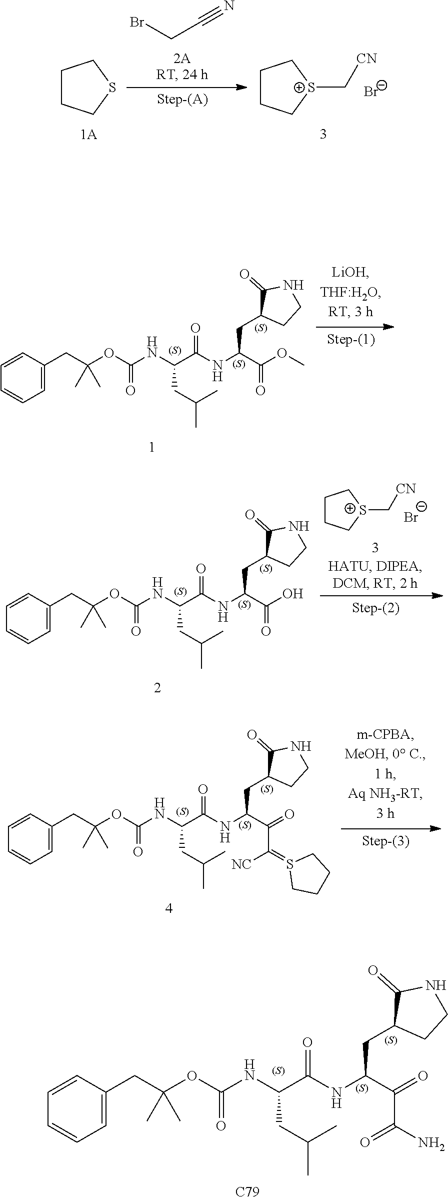

### 1-(Cyanomethyl) tetrahydro-1H-thiophen-1-iumbromide (3)

To a stirred solution of tetrahydrothiophene (1A) (10 g, 113.63 mmol) was added bromoacetonitrile (2A) (13.5 g, 113.63 mmol) and stirred at room temperature for 24 h. The resulting solids were washed with diethyl ether and dried to afford 1-(cyanomethyl) tetrahydro-1H-thiophen-1-iumbromide (3). LCMS (ESI): m/z 205.0 [M−H]⁺

### (S)-2-((S)-4-methyl-2-((((2-methyl-1-phenylpropan-2-yl) oxy) carbonyl) amino) pentanamido)-3-((S)-2-oxopyrrolidin-3-yl) propanoic acid (2)

To a stirred solution of methyl (S)-2-((S)-4-methyl-2-((((2-methyl-1-phenylpropan-2-yl)oxy)carbonyl)amino)pentanamido)-3-((S)-2-oxopyrrolidin-3-yl)propanoate (1) (800 mg, 1.68 mmol) in THF (6 mL), water (3 mL) was added lithium hydroxide (80 mg, 3.36 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (S)-2-((S)-4-methyl-2-((((2-methyl-1-phenylpropan-2-yl)oxy)carbonyl)amino)pentanamido)-3-((S)-2-oxopyrrolidin-3-yl)propanoic acid (2). TLC system: 10% MeOH in DCM Rf: 0.2 LCMS (ESI): m/z 484.15 [M+Na]⁺

### 2-Methyl-1-phenylpropan-2-yl((S)-1-(((S)-4-cyano-3-oxo-1-((S)-2-oxopyrrolidin-3-yl)-4-(tetrahydro-1l4-thiophen-1-ylidene) butan-2-yl) amino)-4-methyl-1-oxopentan-2-yl) carbamate (4)

To a stirred solution of (S)-2-((S)-4-methyl-2-((((2-methyl-1-phenylpropan-2-yl)oxy)carbonyl)amino)pentanamido)-3-((S)-2-oxopyrrolidin-3-yl)propanoic acid (3) (700 mg, 1.51 mmol) DCM (15 mL) added HATU (1.14 g, 3.02 mmol), DIPEA (0.8 mL, 4.53 mmol) and 1-(cyanomethyl)tetrahydro-1H-thiophen-1-iumbromide (3) (376 mg, 1.82 mmol) at 0° C. simultaneously and stirred at room temperature for 2 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 2-methyl-1-phenylpropan-2-yl((S)-1-(((S)-4-cyano-3-oxo-1-((S)-2-oxopyrrolidin-3-yl)-4-(tetrahydro-1l4-thiophen-1-ylidene) butan-2-yl) amino)-4-methyl-1-oxopentan-2-yl)carbamate (4). TLC system: 10% Methanol in dichloromethane Rf: 0.4 LCMS (ESI): m/z 571.78 [M+H]⁺

### 2-Methyl-1-phenylpropan-2-yl((S)-1-(((S)-4-amino-3,4-dioxo-1-((S)-2-oxopyrrolidin-3-yl) butan-2-yl) amino)-4-methyl-1-oxopentan-2-yl) carbamate (Compound C79)

To a stirred solution of 2-methyl-1-phenylpropan-2-yl((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (150 mg, 0.26 mmol) in methanol (3 mL) was added mCPBA (90 mg, 0.52 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford 2-methyl-1-phenylpropan-2-yl((S)-1-(((S)-4-amino-3,4-dioxo-1-((S)-2-oxopyrrolidin-3-yl) butan-2-yl)amino)-4-methyl-1-oxopentan-2-yl)carbamate (Compound C79). TLC system: 10% Methanol in dichloromethane Rf: 0.2 LCMS (ESI): m/z 448.49 [M+H]⁺

### Example 66: Synthesis of Compounds C80 and C98

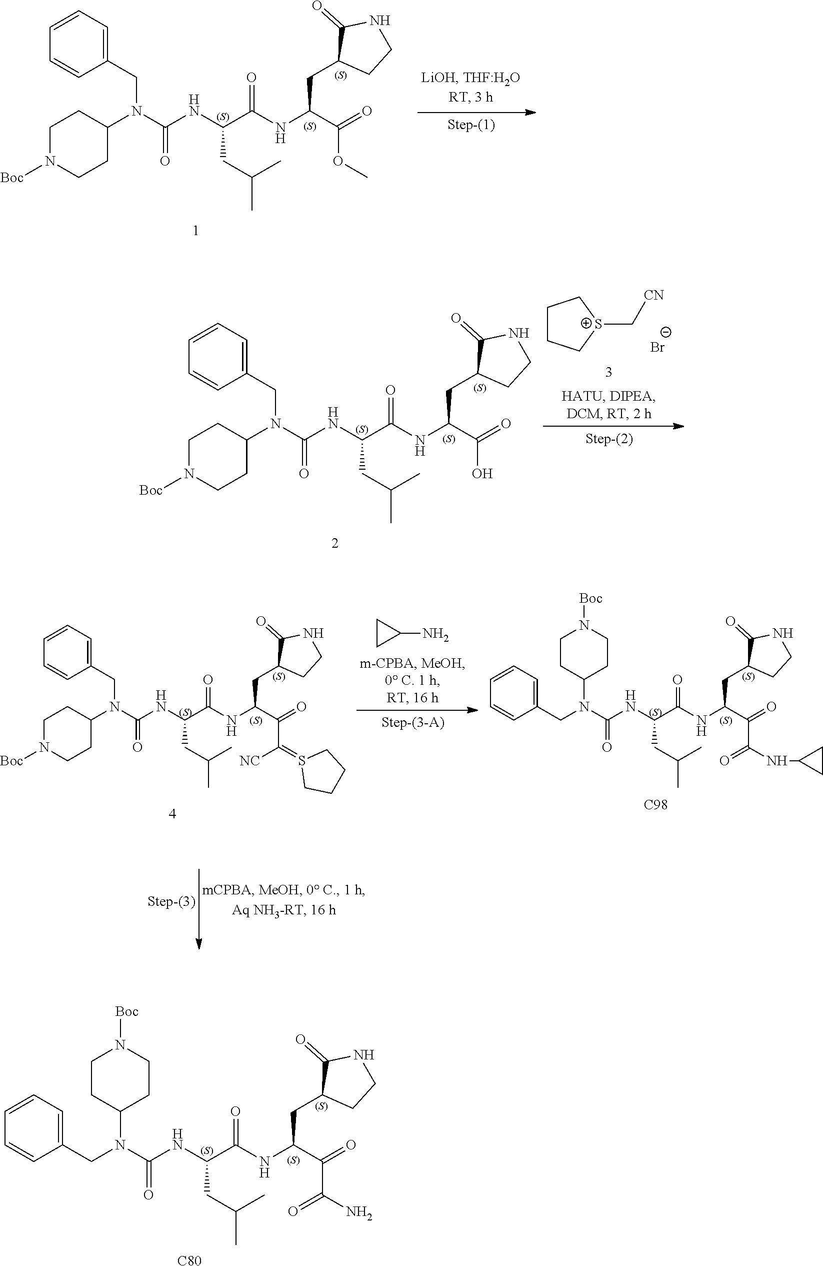

### (S)-2-((S)-2-(3-benzyl-3-(1-(tert-butoxycarbonyl)piperidin-4-yl)ureido)-4-methylpentanamido)-3-((S)-2-oxopyrrolidin-3-yl)propanoic acid (2)

To a stirred solution of tert-butyl 4-(1-benzyl-3-((S)-1-(((S)-1-methoxy-1-oxo-3-((S)-2-oxopyrrolidin-3-yl)propan-2-yl)amino)-4-methyl-1-oxopentan-2-yl)ureido)piperidine-1-carboxylate (1) (2 g, 3.252 mmol) in THF (15 mL), water (5 mL) was added lithium hydroxide (234 mg, 3.36 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×20 mL), dried over sodium sulfate, concentrated under reduced pressure to afford (S)-2-((S)-2-(3-benzyl-3-(1-(tert-butoxycarbonyl)piperidin-4-yl)ureido)-4-methylpentanamido)-3-((S)-2-oxopyrrolidin-3-yl)propanoic acid (2). TLC system: 10% MeOH in DCM Rf: 0.2 LCMS (ESI): m/z 601.4 [M+H]⁺

### Tert-butyl 4-(1-benzyl-3-((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)ureido)piperidine-1-carboxylate (4)

To a stirred solution of (S)-2-((S)-2-(3-benzyl-3-(1-(tert-butoxycarbonyl)piperidin-4-yl)ureido)-4-methylpentanamido)-3-((S)-2-oxopyrrolidin-3-yl)propanoic acid (2) (1.5 g, 2.495 mmol) in DCM (20 mL) added HATU (1.42 g, 3.743 mmol), DIPEA (1.37 mL, 7.487 mmol) and 1-(cyanomethyl)tetrahydro-1H-thiophen-1-iumbromide (3) (616 mg, 2.995 mmol) at 0° C. simultaneously and stirred at room temperature for 2 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford tert-butyl 4-(1-benzyl-3-((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)ureido)piperidine-1-carboxylate (4). TLC system: 10% Methanol in dichloromethane Rf: 0.4 LCMS (ESI): m/z 711.53 [M+H]⁺

### Tert-butyl 4-(3-((S)-1-(((S)-4-amino-3,4-dioxo-1-((S)-2-oxopyrrolidin-3-yl)butan-2-yl)amino)-4-methyl-1-oxopentan-2-yl)-1-benzoylureido)piperidine-1-carboxylate (Compound 080)

To a stirred solution of tert-butyl 4-(1-benzyl-3-((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)ureido)piperidine-1-carboxylate (4) (150 mg, 0.211 mmol) in methanol (3 mL) was added mCPBA (72 mg, 0.422 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep-HPLC to afford tert-butyl 4-(3-((S)-1-(((S)-4-amino-3,4-dioxo-1-((S)-2-oxopyrrolidin-3-yl)butan-2-yl)amino)-4-methyl-1-oxopentan-2-yl)-1-benzoylureido)piperidine-1-carboxylate (Compound C80). TLC system: 10% Methanol in dichloromethane Rf: 0.3 LCMS (ESI): m/z 629.3 [M+H]⁺

### Tert-butyl 4-(1-benzyl-3-((S)-1-(((S)-4-(cyclopropylamino)-3,4-dioxo-1-((S)-2-oxopyrrolidin-3-yl)butan-2-yl)amino)-4-methyl-1-oxopentan-2-yl)ureido)piperidine-1-carboxylate (Compound C98)

To a stirred solution of tert-butyl 4-(1-benzyl-3-((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)ureido)piperidine-1-carboxylate (4) (200 mg, 0.281 mmol) in methanol (5 mL) was added m-CPBA (121 mg, 0.704 mmol) at 0° C. and the reaction mixture stirred for 2 h at 0° C. and added cyclopropanamine (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep-HPLC to afford tert-butyl 4-(1-benzyl-3-((S)-1-(((S)-4-(cyclopropylamino)-3,4-dioxo-1-((S)-2-oxopyrrolidin-3-yl)butan-2-yl)amino)-4-methyl-1-oxopentan-2-yl)ureido)piperidine-1-carboxylate (Compound C98). TLC system: 10% Methanol in dichloromethane Rf: 0.3 LCMS (ESI): m/z 669.3 [M+H]⁺

### Example 67: Synthesis of Compound C86

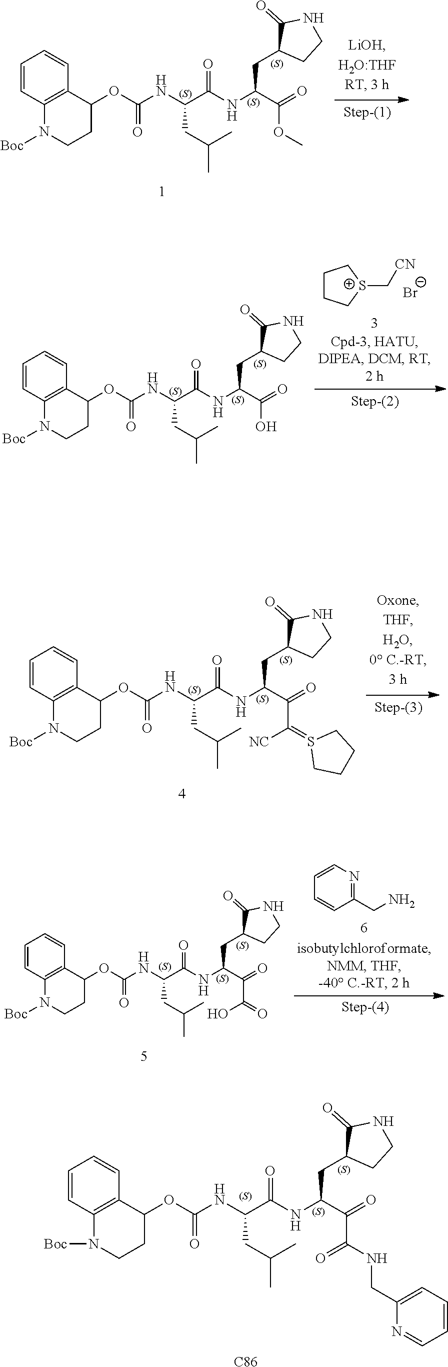

### (2S)-2-((2S)-2-((((1-(tert-butoxycarbonyl)-1, 2, 3, 4-tetrahydroquinolin-4-yl) oxy) carbonyl) amino)-4-methylpentanamido)-3-((S)-2-oxopyrrolidin-3-yl) propanoic acid (2)

To a stirred solution of tert-butyl 4-((((S)-1-(((S)-1-methoxy-1-oxo-3-((S)-2-oxopyrrolidin-3-yl)propan-2-yl)amino)-4-methyl-1-oxopentan-2-yl)carbamoyl)oxy)-3,4-dihydroquinoline-1(2H)-carboxylate (1) (400 mg, 0.69 mmol) in THF (4 mL), water (2 mL) was added lithium hydroxide (50 mg, 2.08 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×15 mL), dried over sodium sulfate, concentrated under reduced pressure to afford (2S)-2-((2S)-2-((((1-(tert-butoxycarbonyl)-1,2,3,4-tetrahydroquinolin-4-yl)oxy)carbonyl)amino)-4-methylpentanamido)-3-((S)-2-oxopyrrolidin-3-yl)propanoic acid (2). TLC system: 10% Methanol in dichloromethane Rf: 0.1 LCMS (ESI): m/z 559.65 [M−H]⁺

### Tert-butyl 4-((((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamoyl)oxy)-3,4-dihydroquinoline-1(2H)-carboxylate (4)

To a stirred solution of (2S)-2-((2S)-2-((((1-(tert-butoxycarbonyl)-1,2,3,4-tetrahydroquinolin-4-yl)oxy)carbonyl)amino)-4-methylpentanamido)-3-((S)-2-oxopyrrolidin-3-yl)propanoic acid (2) (350 mg, 0.62 mmol) in DCM (10 mL) added HATU (474 mg, 1.24 mmol), DIPEA (0.3 mL, 1.87 mmol) and methyl 1-(cyanomethyl)tetrahydro-1H-thiophen-1-ium bromide (3) (155 mg, 0.74 mmol) at 0° C. simultaneously and stirred at room temperature for 2 h. Reaction mixture was quenched with ice water (20 mL), extracted with dichloromethane (2×15 mL), dried over sodium sulfate and evaporated under reduced pressure. The crude residue was purified by combi-flash NP, compound eluted at 3% methanol in dichloromethane to afford tert-butyl 4-((((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamoyl)oxy)-3,4-dihydroquinoline-1(2H)-carboxylate (4). TLC system: 10% Methanol in dichloromethane Rf: 0.4 LCMS (ESI): m/z 670.48 [M+H]⁺

### (3S)-3-((2S)-2-((((1-(tert-butoxycarbonyl)-1, 2, 3, 4-tetrahydroquinolin-4-yl) oxy) carbonyl) amino)-4-methylpentanamido)-2-oxo-4-((S)-2-oxopyrrolidin-3-yl) butanoic acid (5)

To a stirred solution of tert-butyl 4-((((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamoyl)oxy)-3,4-dihydroquinoline-1(2H)-carboxylate (4) (300 mg, 0.44 mmol) in THF (5 mL), water (2 mL) was added oxone (826 mg, 1.34 mmol) at RT and stirred at room temperature for 3 h. The progress of the reaction was monitored by TLC and LCMS. Reaction mixture completely distilled under reduced pressure and extracted with ethyl acetate (2×20 mL), dried over sodium sulfate, concentrated under reduced pressure to afford (3S)-3-((2S)-2-((((1-(tert-butoxycarbonyl)-1,2,3,4-tetrahydroquinolin-4-yl)oxy)carbonyl)amino)-4-methylpentanamido)-2-oxo-4-((S)-2-oxopyrrolidin-3-yl)butanoic acid (5). TLC system: 10% Methanol in dichloromethane Rf: 0.1 LCMS (ESI): m/z 587.37 [M−H]⁺

### Tert-butyl 4-((((S)-1-(((S)-3,4-dioxo-1-((S)-2-oxopyrrolidin-3-yl)-4-((pyridin-2-ylmethyl)amino) butan-2-yl)amino)-4-methyl-1-oxopentan-2-yl)carbamoyl)oxy)-3,4-dihydroquinoline-1(2H)-carboxylate (Compound C86)

To a stirred solution of (3S)-3-((2S)-2-((((1-(tert-butoxycarbonyl)-1,2,3,4-tetrahydroquinolin-4-yl)oxy)carbonyl)amino)-4-methylpentanamido)-2-oxo-4-((S)-2-oxopyrrolidin-3-yl)butanoic acid (5) (130 mg, 0.22 mmol) was dissolved in THF (6 mL) was added NMM (29 mg, 0.28 mmol), isobutylchloroformate (36 mg, 0.26 mmol) and followed by pyridin-2-ylmethanamine (6) (30 mg, 0.26 mmol) at −40° C. and stirred at RT for 2 h. Reaction mixture was diluted with ethyl acetate (10 mL) and washed with brine solution (2×15 mL). Organic layer was dried over anhydrous Na₂SO₄, filtered and concentrated to get crude compound. The crude compound was purified by prep HPLC to afford tert-butyl 4-((((S)-1-(((S)-3,4-dioxo-1-((S)-2-oxopyrrolidin-3-yl)-4-((pyridin-2-ylmethyl)amino) butan-2-yl)amino)-4-methyl-1-oxopentan-2-yl) carbamoyl) oxy)-3,4-dihydroquinoline-1(2H)-carboxylate (Compound C86). TLC system: 10% Methanol in dichloromethane Rf: 0.3 LCMS (ESI): m/z 679.3 (M+H)⁺

### Example 68: Synthesis of Compounds C90 and C87

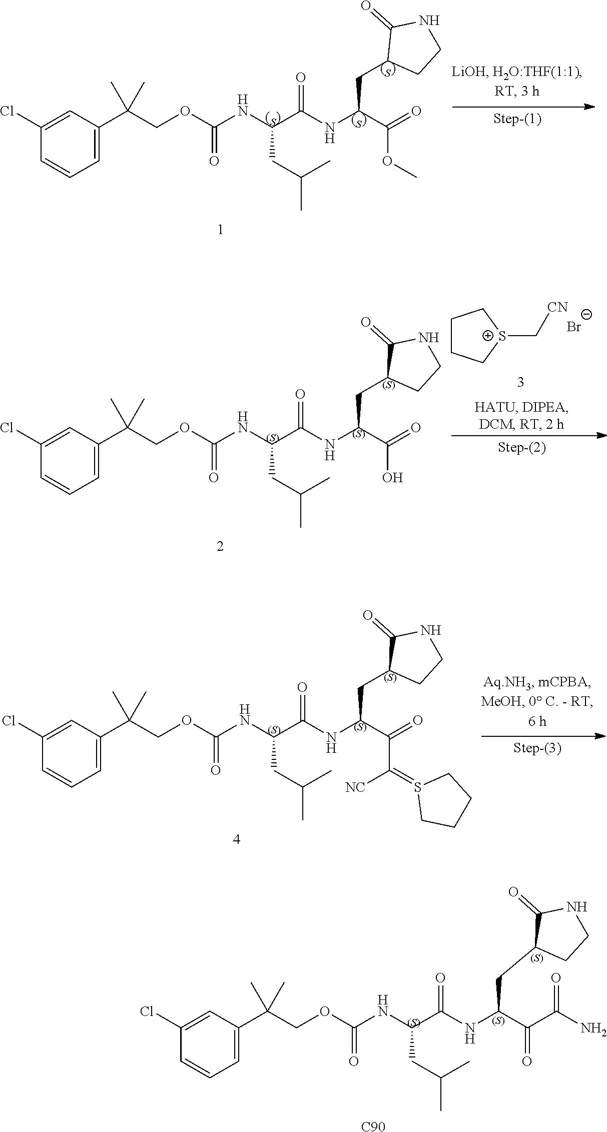

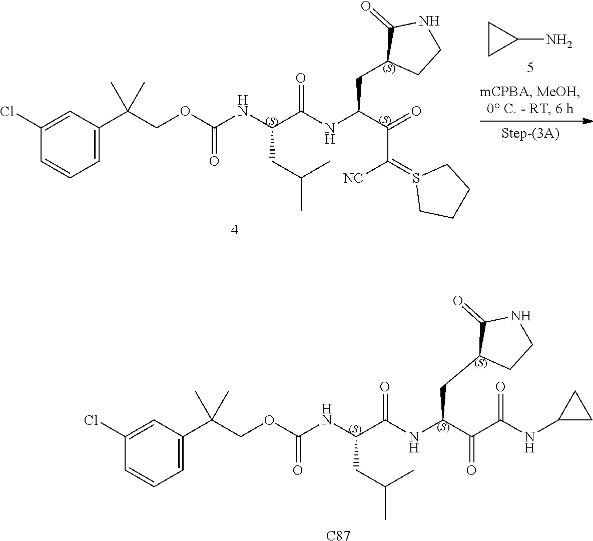

### (S)-2-((S)-2-(((2-(3-chlorophenyl)-2-methylpropoxy)carbonyl)amino)-4-methylpentanamido)-3-((S)-2-oxopyrrolidin-3-yl)propanoic acid (2)

To a stirred solution of methyl (S)-2-((S)-2-(((2-(3-chlorophenyl)-2-methylpropoxy)carbonyl)amino)-4-methylpentanamido)-3-((S)-2-oxopyrrolidin-3-yl)propanoate (1) (2 g, 3.9292 mmol) in THF (20 mL), water (10 mL) was added lithium hydroxide (282 mg, 11.7878 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (S)-2-((S)-2-(((2-(3-chlorophenyl)-2-methylpropoxy)carbonyl)amino)-4-methylpentanamido)-3-((S)-2-oxopyrrolidin-3-yl)propanoic acid (2). TLC system: 10% MeOH in DCM Rf: 0.2 LCMS (ESI): m/z 496.69 [M+H]⁺

### 2-(3-chlorophenyl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4)

To a stirred solution of (S)-2-((S)-2-(((2-(3-chlorophenyl)-2-methylpropoxy)carbonyl)amino)-4-methylpentanamido)-3-((S)-2-oxopyrrolidin-3-yl)propanoic acid (2) (1.1 g, 2.2222 mmol) DCM (15 mL) added HATU (380 mg, 3.3333 mmol), DIPEA (1.22 mL, 6.666 mmol) and 1-(cyanomethyl)tetrahydro-1H-thiophen-1-iumbromide (3) (915 mg, 4.444 mmol) at 0° C. simultaneously and stirred at room temperature for 2 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 2-(3-chlorophenyl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4). TLC system: 10% Methanol in dichloromethane Rf: 0.4 LCMS (ESI): m/z 605.71 [M+H]⁺

### 2-(3-chlorophenyl)-2-methylpropyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C90)

To a stirred solution of 2-(3-chlorophenyl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (350 mg, 0.579 mmol) in methanol (5 mL) was added mCPBA (200 mg, 1.158 mmol) at 0° C. and the reaction mixture stirred for 2 h at 0° C. and added aq ammonia (1.5 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford 2-(3-chlorophenyl)-2-methylpropyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C90). TLC system: 10% Methanol in dichloromethane Rf: 0.2 LCMS (ESI): m/z 523.2 [M+H]⁺

### 2-(3-Chlorophenyl)-2-methylpropyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C87)

To a stirred solution of 2-(3-chlorophenyl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (150 mg, 0.248 mmol) in methanol (5 mL) was added m-CPBA (85 mg, 0.496 mmol) at 0° C. and the reaction mixture stirred for 2 h at 0° C. and added cyclopropanamine (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep-HPLC to afford 2-(3-chlorophenyl)-2-methylpropyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C87). TLC system: 10% Methanol in dichloromethane Rf: 0.3 LCMS (ESI): m/z 563.2 [M+H]⁺

### Example 69: Synthesis of Compound C88

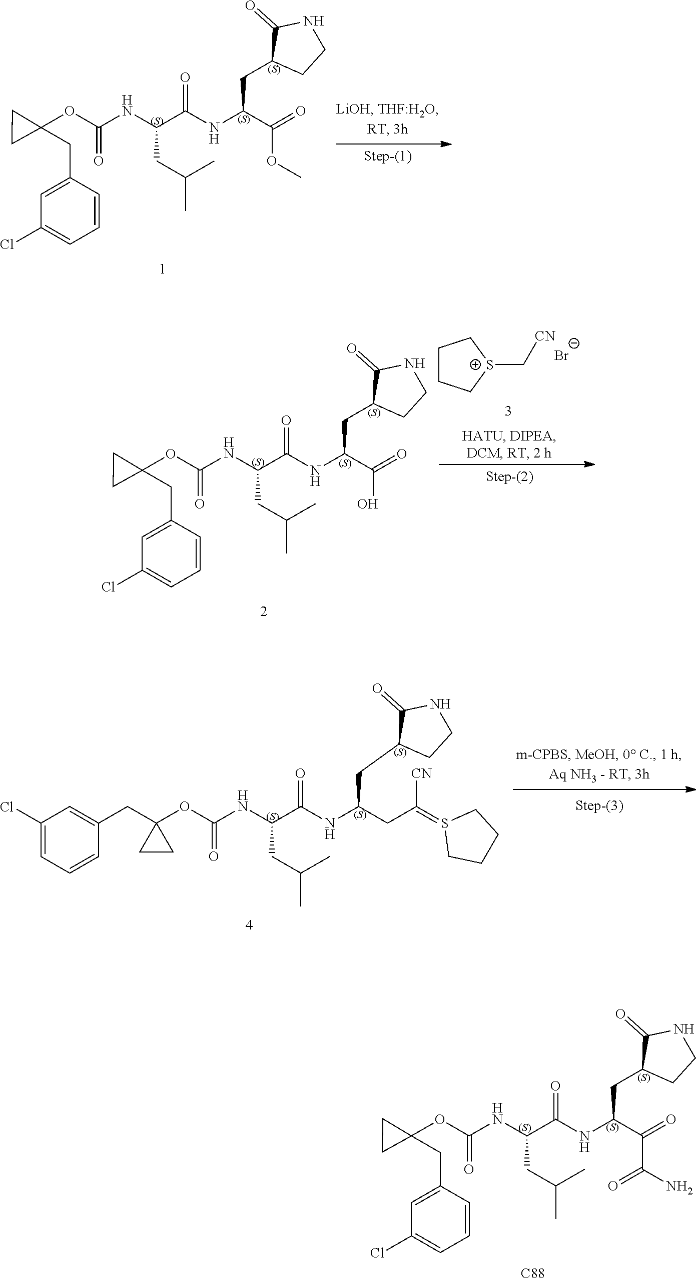

### (S)-2-((S)-2-(((1-(3-Chlorobenzyl) cyclopropoxy) carbonyl) amino)-4-methylpentanamido)-3-((S)-2-oxopyrrolidin-3-yl) propanoic acid (2)

To a stirred solution of methyl (S)-2-((S)-2-(((1-(3-chlorobenzyl)cyclopropoxy) carbonyl)amino)-4-methylpentanamido)-3-((S)-2-oxopyrrolidin-3-yl)propanoate (1) (2.6 g, 5.12 mmol) in THF (30 mL), water (15 mL) was added lithium hydroxide (629 mg, 15.36 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×60 mL), dried over sodium sulfate, concentrated under reduced pressure to afford (S)-2-((S)-2-(((1-(3-chlorobenzyl)cyclopropoxy)carbonyl)amino)-4-methylpentanamido)-3-((S)-2-oxopyrrolidin-3-yl)propanoic acid (2). TLC system: 10% MeOH in DCM Rf: 0.2 LCMS (ESI): m/z 494.68 [M+H]⁺

### 1-(3-Chlorobenzyl) cyclopropyl ((S)-1-(((S)-4-cyano-3-oxo-1-((S)-2-oxopyrrolidin-3-yl)-4-(tetrahydro-1l4-thiophen-1-ylidene) butan-2-yl) amino)-4-methyl-1-oxopentan-2-yl) carbamate

To a stirred solution of (S)-2-((S)-2-(((1-(3-chlorobenzyl)cyclopropoxy)carbonyl)amino)-4-methylpentanamido)-3-((S)-2-oxopyrrolidin-3-yl)propanoic acid (2) (1.5 g, 3.04 mmol) DCM (20 mL) added HATU (1.7 g, 4.56 mmol), DIPEA (1.3 mL, 9.12 mmol) and 1-(cyanomethyl)tetrahydro-1H-thiophen-1-iumbromide (3) (940 mg, 4.56 mmol) at 0° C. simultaneously and stirred at room temperature for 2 h. Reaction mixture was diluted with ice water (50 mL), extracted with dichloromethane (2×60 mL), dried over sodium sulfate and evaporated under reduced pressure. The crude residue was purified by combi-flash NP, compound eluted at 5% methanol in dichloromethane to afford 1-(3-chlorobenzyl)cyclopropyl((S)-1-(((S)-4-cyano-3-oxo-1-((S)-2-oxopyrrolidin-3-yl)-4-(tetrahydro-1l4-thiophen-1-ylidene) butan-2-yl)amino)-4-methyl-1-oxopentan-2-yl)carbamate (4). TLC system: 10% Methanol in dichloromethane Rf: 0.4 LCMS (ESI): m/z 603.74 [M+H]⁺

### 1-(3-Chlorobenzyl) cyclopropyl ((S)-1-(((S)-4-amino-3,4-dioxo-1-((S)-2-oxopyrrolidin-3-yl) butan-2-yl) amino)-4-methyl-1-oxopentan-2-yl)carbamate (Compound C88)

To a stirred solution of 1-(3-chlorobenzyl)cyclopropyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (200 mg, 0.33 mmol) in methanol (3 mL) was added mCPBA (114 mg, 0.66 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×20 mL). Organic layer was washed with brine solution (30 mL), dried over Na₂SO₄ and concentrated to get crude compound. The crude compound was purified by prep HPLC to afford 1-(3-chlorobenzyl)cyclopropyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C88). TLC system: 10% Methanol in dichloromethane Rf: 0.2 LCMS (ESI): m/z 521.2 [M+H]⁺

### Example 70: Synthesis of Compound C89

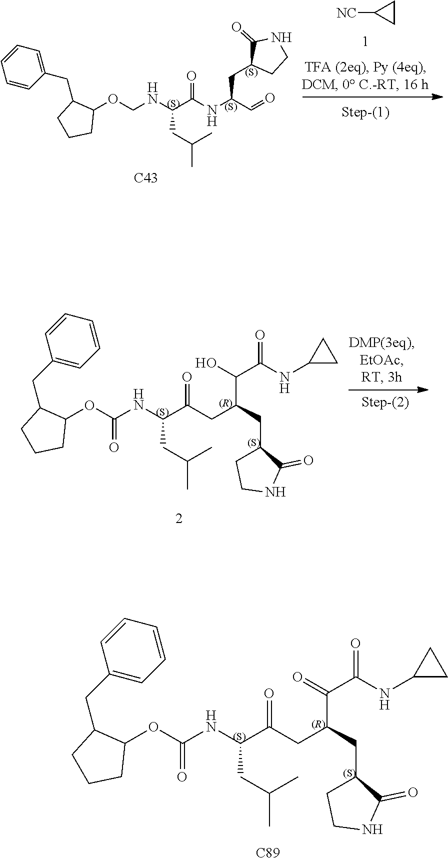

### 2-Benzylcyclopentyl ((2S)-1-(((2R)-4-(cyclopropylamino)-3-hydroxy-4-oxo-1-((S)-2-oxopyrrolidin-3-yl)butan-2-yl)amino)-4-methyl-1-oxopentan-2-yl)carbamate (2)

To a stirred solution of 2-benzylcyclopentyl ((S)-4-methyl-1-oxo-1-(((S)-1-oxo-3-((S)-2-oxopyrrolidin-3-yl)propan-2-yl)amino)pentan-2-yl)carbamate (Compound C43) (300 mg, 0.636 mmol) was dissolved in DCM (10 mL) added Pyridine (0.2 mL, 2.547 mmol), isocyanocyclopropane (1) (51 mg, 0.764 mmol) sequentially at 0° C. stirred for 10 min added TFA (0.09 mL, 1.273 mmol) at 0° C. and stirred at RT for 16 h. The progress of the reaction was monitored by TLC and LCMS. Reaction mixture was quenched with ice water (100 mL), extracted with EtOAc (2×55 mL), the combined organic layers was washed with 1N HCl (3×20 mL) brine solution (3×20 mL) organic layers was dried over anhydrous Na₂SO₄ and evaporated under reduced pressure. The crude residue 2-benzylcyclopentyl ((2S)-1-(((2R)-4-(cyclopropyl amino)-3-hydroxy-4-oxo-1-((S)-2-oxopyrrolidin-3-yl)butan-2-yl)amino)-4-methyl-1-oxopentan-2-yl)carbamate (2). TLC system: 10% Methanol/Dichloromethane Rf: 0.5 LCMS (ESI): m/z 557.3 [M+H]⁺

### 2-Benzylcyclopentyl ((S)-1-(((R)-4-(cyclopropylamino)-3,4-dioxo-1-((S)-2-oxopyrrolidin-3-yl)butan-2-yl)amino)-4-methyl-1-oxopentan-2-yl)carbamate (Compound C89)

To a stirred solution of 2-benzylcyclopentyl ((2S)-1-(((2R)-4-(cyclopropylamino)-3-hydroxy-4-oxo-1-((S)-2-oxopyrrolidin-3-yl)butan-2-yl)amino)-4-methyl-1-oxopentan-2-yl)carbamate (2) (250 mg, 0.449 mmol) in EtOAc (10 mL) at 0° C. was added Dess-Martin periodinane (571 mg, 1.34 mmol) and stirred at RT for 16 h. The progress of the reaction was monitored by TLC and LCMS. Reaction mixture was filter through celite pad and washed with Ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. It was purified by prep HPLC to afford 2-benzylcyclopentyl ((S)-1-(((R)-4-(cyclo propyl amino)-3,4-dioxo-1-((S)-2-oxopyrrolidin-3-yl) butan-2-yl)amino)-4-methyl-1-oxopentan-2-yl)carbamate (Compound C89). TLC system: 10% Methanol/Dichloromethane Rf: 0.4 LCMS (ESI): m/z 555.3 (M+H)⁺

### Example 71: Synthesis of Compound C93

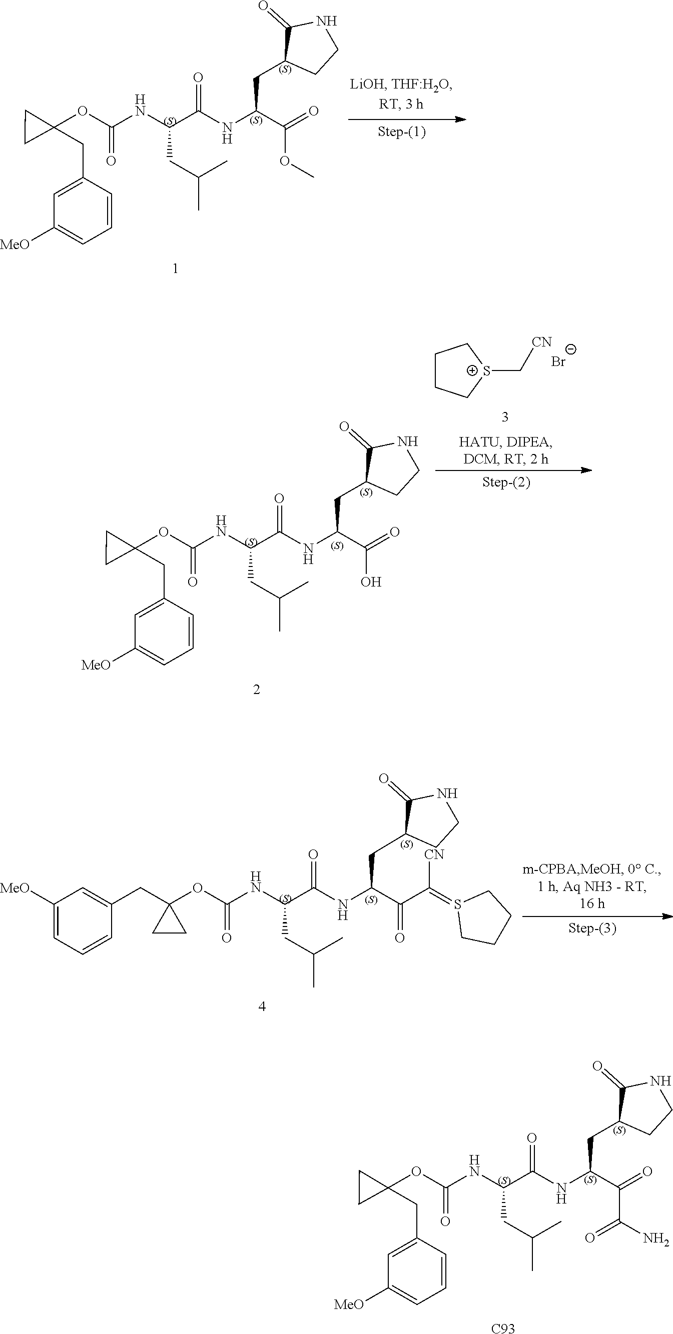

### (S)-2-((S)-2-(((1-(3-Methoxybenzyl) cyclopropoxy) carbonyl) amino)-4-methylpentanamido)-3-((S)-2-oxopyrrolidin-3-yl) propanoic acid (2)

To a stirred solution of methyl (S)-2-((S)-2-(((1-(3-methoxybenzyl) cyclopropoxy) carbonyl)amino)-4-methylpentanamido)-3-((S)-2-oxopyrrolidin-3-yl)propanoate (1) (1.8 g, 3.57 mmol) in THF (30 mL), water (15 mL) was added lithium hydroxide (440 mg, 10.73 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×40 mL), dried over sodium sulfate, concentrated under reduced pressure to afford (S)-2-((S)-2-(((1-(3-methoxybenzyl)cyclopropoxy)carbonyl)amino)-4-methylpentanamido)-3-((S)-2-oxopyrrolidin-3-yl)propanoic acid (2). TLC system: 10% MeOH in DCM Rf: 0.2 LCMS (ESI): m/z 490.69[M+H]⁺

### 1-(3-Methoxybenzyl)cyclopropyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4)

To a stirred solution of (S)-2-((S)-2-(((1-(3-methoxybenzyl) cyclopropoxy)carbonyl) amino)-4-methylpentanamido)-3-((S)-2-oxopyrrolidin-3-yl)propanoic acid (2) (1.5 g, 3.06 mmol) DCM (20 mL) added HATU (1.7 g, 4.60 mmol), DIPEA (1.3 mL, 9.20 mmol) and 1-(cyanomethyl)tetrahydro-1H-thiophen-1-iumbromide (3) (947 mg, 4.60 mmol) at 0° C. simultaneously and stirred at room temperature for 2 h. Reaction mixture was diluted with ice water (50 mL), extracted with dichloromethane (2×40 mL), dried over sodium sulfate and evaporated under reduced pressure. The crude residue was purified by combi-flash NP, compound eluted at 5% methanol in dichloromethane to afford 1-(3-methoxybenzyl)cyclopropyl ((S)-1-(((S)-4-cyano-3-oxo-1-((S)-2-oxopyrrolidin-3-yl)-4-(tetrahydro-1l4-thiophen-1-ylidene) butan-2-yl)amino)-4-methyl-1-oxopentan-2-yl) carbamate (4). TLC system: 10% Methanol in dichloromethane Rf: 0.4 LCMS (ESI): m/z 599.26 [M+H]⁺

### 1-(3-Methoxybenzyl) cyclopropyl ((S)-1-(((S)-4-amino-3,4-dioxo-1-((S)-2-oxopyrrolidin-3-yl) butan-2-yl) amino)-4-methyl-1-oxopentan-2-yl) carbamate (Compound C93)

To a stirred solution of 1-(3-methoxybenzyl)cyclopropyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (300 mg, 0.50 mmol) in methanol (3 mL) was added m-CPBA (172 mg, 1.00 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×20 mL). Organic layer was washed with brine solution (30 mL), dried over Na₂SO₄ and concentrated to get crude compound. The crude compound was purified by prep HPLC to afford 1-(3-methoxybenzyl)cyclopropyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C93). TLC system: 10% Methanol in dichloromethane Rf: 0.2 LCMS (ESI): m/z 517.2 [M+H]⁺

### Example 72: Synthesis of Compound C96

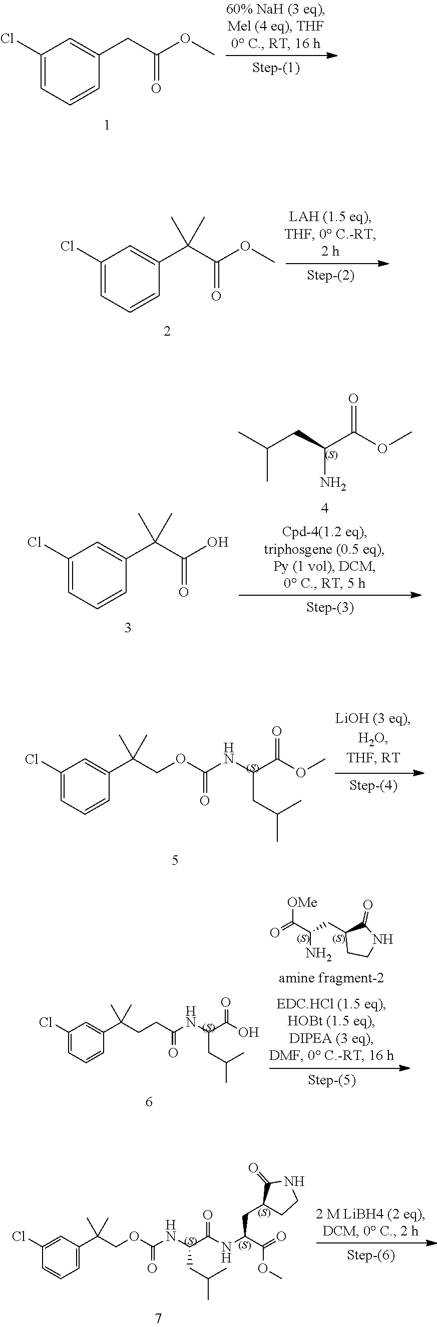

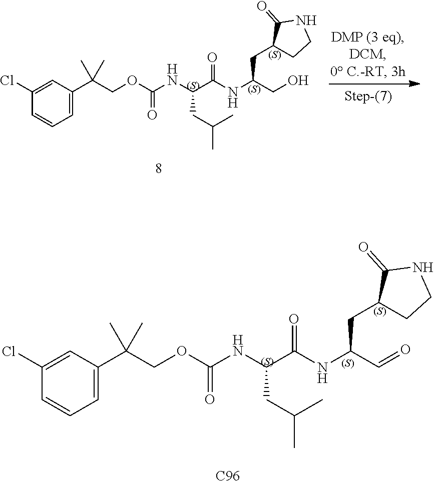

### Methyl 2-(3-chlorophenyl)-2-methylpropanoate (2)

To a stirred solution of methyl 2-(3-chlorophenyl)acetate (1) (10 g, 54.17 mmol) in THF (100 mL) was added 60% NaH (6.5 g, 162.51 mmol) at 0° C. and stirred for 15 min then added Mel (13.5 mL, 216.68 mmol) at same temperature and allowed to RT for 16 h. The progress of the reaction was monitored by TLC, Reaction mixture was quenched with sat. ammonium chloride and extracted with EtOAc (2×100 mL), combined the organic layer and washed with brine solution (100 mL), dried over sodium sulfate, filtered and evaporated under reduced pressure to afford crude, residue was purified by combi-flash, compound eluted at 5% ethyl acetate in pet ether to afford methyl 2-(3-chlorophenyl)-2-methylpropanoate (2). TLC system: 10% Ethyl acetate in Pet ether Rf: 0.3 LCMS (ESI): m/z 213.31 [M+H]⁺

### 2-(3-Chlorophenyl)-2-methylpropan-1-ol (3)

To a stirred solution of methyl 2-(3-chlorophenyl)-2-methylpropanoate (6 g, 28.30 mmol), in THF (80 mL) was added slowly 2.4 M LAH in THF (11.7 mL, 28.30 mmol) at −50° C. and stirred for 1 h. The progress of the reaction was monitored by TLC. Reaction mixture was quenched with sat. ammonium chloride and extracted with EtOAc (2×50 mL), combined the organic layer and washed with brine solution (60 mL), dried over sodium sulfate, filtered and evaporated under reduced pressure to afford crude, residue was purified by combi-flash, compound eluted at 10% ethyl acetate in pet ether to afford 2-(3-chlorophenyl)-2-methylpropan-1-ol (3). TLC system: 30% Ethyl acetate Rf: 0.3 LCMS (ESI): m/z=167.4 [M−OH]⁺

### Methyl ((2-(3-chlorophenyl)-2-methylpropoxy) carbonyl)-L-leucinate (5)

To a stirred solution of 2-(3-chlorophenyl)-2-methylpropan-1-ol (3) (2.8 g, 15.16 mmol), methyl L-leucinate (4) (2.64 g, 18.19 mmol) in DCM (40 mL) was added pyridine (3 mL, 1 vol) followed by triphosgene (2.24 g, 7.58 mmol) at 0° C. and stirred at room temperature for 3 h. The progress of the reaction was monitored by TLC and LCMS. Reaction mixture was quenched with ice water (30 mL), extracted with DCM (2×30 mL). The combined organic layer was dried over sodium sulfate, filtered and evaporated under reduced pressure. The crude residue was purified by combi-flash, compound eluted at 8% ethyl acetate in pet ether to afford methyl ((2-(3-chlorophenyl)-2-methylpropoxy) carbonyl)-L-leucinate (5). TLC system: 30% Ethyl acetate in Pet ether R_{f}: 0.5 LCMS (ESI): m/z 356.31 [M+H]⁺

### ((2-(3-Chlorophenyl)-2-methylpropoxy) carbonyl)-L-leucine (6)

To a stirred solution of methyl ((2-(3-chlorophenyl)-2-methylpropoxy) carbonyl)-leucinate (5) (2.3 g, 6.47 mmol) in THF (10 mL) and water (5 mL), was added lithium hydroxide (800 mg, 19.43 mmol) at RT and stirred at room temperature for 3 h. The progress of the reaction was monitored by TLC and LCMS. Reaction mixture completely distilled under reduced pressure and diluted with and washed with diethyl ether (20 mL), aq layer was acidified with aq. 1N HCl solution up to pH˜4, and extracted with dichloromethane (2×30 mL), dried over sodium sulfate, concentrated under reduced pressure to afford ((2-(3-chlorophenyl)-2-methylpropoxy) carbonyl)-L-leucine (6). TLC system: 10% MeOH in DCM R_{f}: 0.1 LCMS (ESI): m/z 342.34 [M+H]⁺

### Methyl (S)-2-((S)-2-(((2-(3-chlorophenyl)-2-methylpropoxy) carbonyl) amino)-4-methylpentanamido)-3-((S)-2-oxopyrrolidin-3-yl) propanoate (7)

To a stirred solution of ((2-(3-chlorophenyl)-2-methylpropoxy) carbonyl)-L-leucine (6) (10 g, 29.32 mmol) in DMF (100 mL) added EDC.HCl (8.401 g, 43.98 mmol), HOBt (5.76 g, 43.98 mmol), DIPEA (15.2 mL, 87.9 mmol) and methyl (S)-2-amino-3-((S)-2-oxopyrrolidin-3-yl)propanoate hydrochloride (amine fragment-2) (6.545 g, 35.19 mmol) at 0° C. simultaneously and stirred at room temperature for 16 h. Reaction mixture was diluted with ice water (100 mL), extracted with ethyl acetate (2×50 mL), dried over sodium sulfate and evaporated under reduced pressure. The crude residue was purified by combi-flash NP, compound eluted at 4% methanol in dichloromethane to afford methyl (S)-2-((S)-2-(((2-(3-chlorophenyl)-2-methylpropoxy)carbonyl)amino)-4-methylpentanamido)-3-((S)-2-oxopyrrolidin-3-yl)propanoate (7). TLC system: 100% Ethyl acetate Rf: 0.4 LCMS (ESI): m/z 510.55 [M+H]⁺

### 2-(3-Chlorophenyl)-2-methylpropyl ((S)-1-(((S)-1-hydroxy-3-((S)-2-oxopyrrolidin-3-yl)propan-2-yl)amino)-4-methyl-1-oxopentan-2-yl)carbamate (8)

To a stirred solution of methyl (S)-2-((S)-2-(((2-(3-chlorophenyl)-2-methylpropoxy)carbonyl) amino)-4-methylpentanamido)-3-((S)-2-oxopyrrolidin-3-yl)propanoate (7) (3 g, 5.893 mmol) in DCM (20 mL) was added 2M LiBH₄ in THF (8.8 mL, 17.681 mmol) at 0° C. and the reaction mixture stirred for 2 h at 0° C. The progress of the reaction was monitored by TLC and LCMS. Reaction mixture was quenched with sat. Ammonium chloride solution (40 mL) and extracted with DCM (2×30 mL). Organic layer was washed with brine solution (30 mL), dried over Na₂SO₄ and concentrated to afford mixture of cpd-8 (2.8 g, 5.81 mmol, 98% yield). The mixture was purified by SFC to afford 2-(3-Chlorophenyl)-2-methylpropyl ((S)-1-(((S)-1-hydroxy-3-((S)-2-oxopyrrolidin-3-yl)propan-2-yl)amino)-4-methyl-1-oxopentan-2-yl)carbamate (8). TLC system: 100% Ethyl acetate Rf: 0.2 LCMS (ESI): m/z 482.50 [M+H]⁺

### 2-(3-Chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C96)

To a stirred solution of 2-methyl-1-phenylpropan-2-yl ((S)-1-(((S)-1-hydroxy-3-((S)-2-oxopyrrolidin-3-yl)propan-2-yl)amino)-4-methyl-1-oxopentan-2-yl)carbamate (8) (1 g, 2.079 mmol) was dissolved in dichloromethane (10 mL) was added Dess-Martin periodinane (2.644 g, 6.237 mmol) at 0° C. and stirred at RT for 2 h. Reaction mixture was diluted with DCM (15 mL) followed by sat. Hypo solution (3×15 mL), followed by sat. NaHCO₃ solution (3×15 mL). Organic layer was dried over anhydrous Na₂SO₄, filtered and concentrated to get crude compound. The crude compound was purified by normal phase silica gel purification 10% MeOH/DCM product was eluted as a 2-(3-chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C96). TLC system: 10% MeOH in DCM Rf: 0.4 LCMS (ESI): m/z 480.2 (M+H)⁺

### Example 73: Synthesis of Compound C97

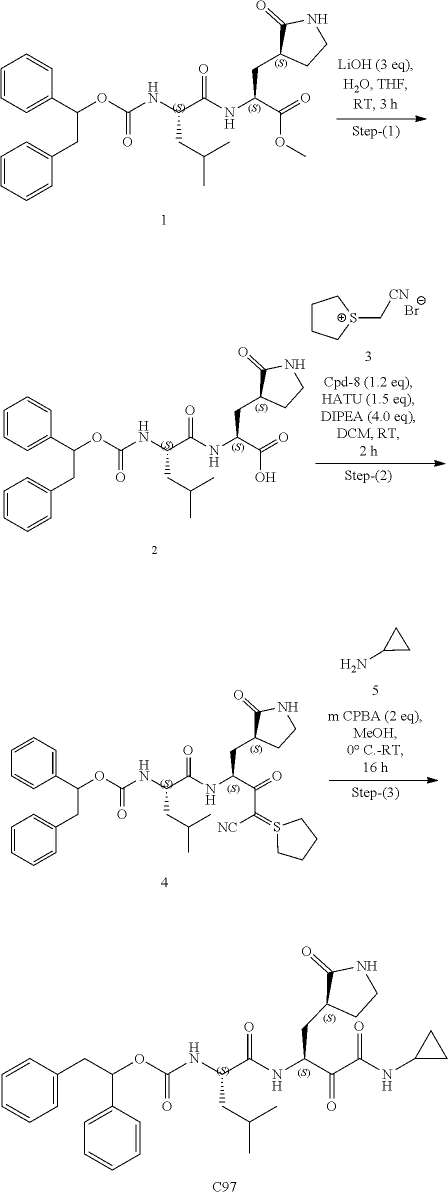

### (2S)-2-((2S)-2-(((1,2-Diphenylethoxy)carbonyl)amino)-4-methylpentanamido)-3-((S)-2-oxopyrrolidin-3-yl)propanoic acid (2)

To a stirred solution of methyl (2S)-2-((2S)-2-(((1,2-diphenylethoxy)carbonyl)amino)-4-methylpentanamido)-3-((S)-2-oxopyrrolidin-3-yl)propanoate (1) (2 g, 3.824 mmol) in THF (20 mL), water (10 mL) was added lithium hydroxide (275 mg, 11.472 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-((2S)-2-(((1,2-diphenylethoxy)carbonyl)amino)-4-methylpentanamido)-3-((S)-2-oxopyrrolidin-3-yl)propanoic acid (2). TLC system: 15% MeOH in DCM Rf: 0.2 LCMS (ESI): m/z 510.67 [M+H]⁺

### 1,2-Diphenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4)

To a stirred solution of (2S)-2-((2S)-2-(((1,2-diphenylethoxy)carbonyl)amino)-4-methyl pentanamido)-3-((S)-2-oxopyrrolidin-3-yl)propanoic acid (2) (1.5 g, 2.946 mmol) DCM (15 mL) added HATU (1.6 g, 4.420 mmol), DIPEA (1.6 mL, 8.840 mmol) and 1-(cyanomethyl)tetra hydro-1H-thiophen-1-iumbromide (3) (728 mg, 3.536 mmol) at 0° C. simultaneously and stirred at room temperature for 2 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 1,2-diphenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4). TLC system: 10% Methanol in dichloromethane Rf: 0.4 LCMS (ESI): m/z 619.51 [M+H]⁺

### 1,2-Diphenylethyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C97)

To a stirred solution of 1,2-diphenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxo pentan-2-yl)carbamate (4) (400 mg, 0.647 mmol) in methanol (5 mL) was added mCPBA (278 mg, 0.618 mmol) at 0° C. and the reaction mixture stirred for 2 h at 0° C. and added cyclopropanamine (2 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep-HPLC to afford 1,2-diphenylethyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C97). TLC system: 10% Methanol in dichloromethane Rf: 0.3 LCMS (ESI): m/z 577.2 [M+H]⁺

### Example 74: Synthesis of Compound C100

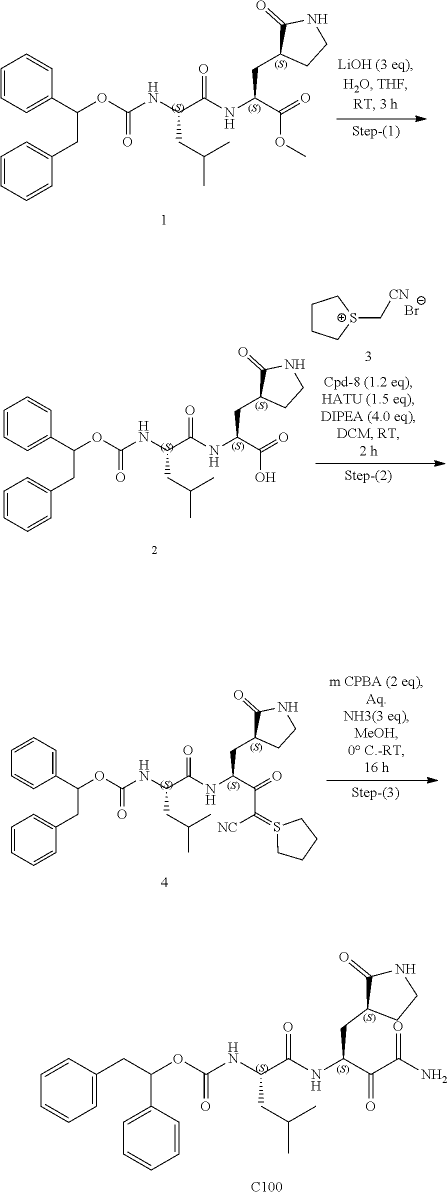

### (2S)-2-((2S)-2-(((1,2-Diphenylethoxy)carbonyl)amino)-4-methylpentanamido)-3-((S)-2-oxopyrrolidin-3-yl)propanoic acid (2)

To a stirred solution of methyl (2S)-2-((2S)-2-(((1,2-diphenylethoxy)carbonyl)amino)-4-methylpentanamido)-3-((S)-2-oxopyrrolidin-3-yl)propanoate (1) (2 g, 3.824 mmol) in THF (20 mL), water (10 mL) was added lithium hydroxide (275 mg, 11.472 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-((2S)-2-(((1,2-diphenylethoxy)carbonyl)amino)-4-methylpentanamido)-3-((S)-2-oxopyrrolidin-3-yl)propanoic acid (2). TLC system: 15% MeOH in DCM Rf: 0.2 LCMS (ESI): m/z 510.67 [M+H]⁺

### 1,2-Diphenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4)

To a stirred solution of (2S)-2-((2S)-2-(((1,2-diphenylethoxy)carbonyl)amino)-4-methyl pentanamido)-3-((S)-2-oxopyrrolidin-3-yl)propanoic acid (2) (1.5 g, 2.946 mmol) DCM (15 mL) added HATU (1.6 g, 4.420 mmol), DIPEA (1.6 mL, 8.840 mmol) and 1-(cyanomethyl)tetra hydro-1H-thiophen-1-iumbromide (3) (728 mg, 3.536 mmol) at 0° C. simultaneously and stirred at room temperature for 2 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 1,2-diphenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4). TLC system: 10% Methanol in dichloromethane Rf: 0.4 LCMS (ESI): m/z 619.51 [M+H]⁺

### 1,2-Diphenylethyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C100)

To a stirred solution of 1,2-diphenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (400 mg, 0.647 mmol) in methanol (5 mL) was added mCPBA (278 mg, 1.618 mmol) at 0° C. and the reaction mixture stirred for 2 h at 0° C. and added aq ammonia (1.5 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford 1,2-diphenylethyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C100). TLC system: 10% Methanol in dichloromethane Rf: 0.3 LCMS (ESI): m/z 537.2 [M+H]⁺

### Example 75: Synthesis of Compound C65

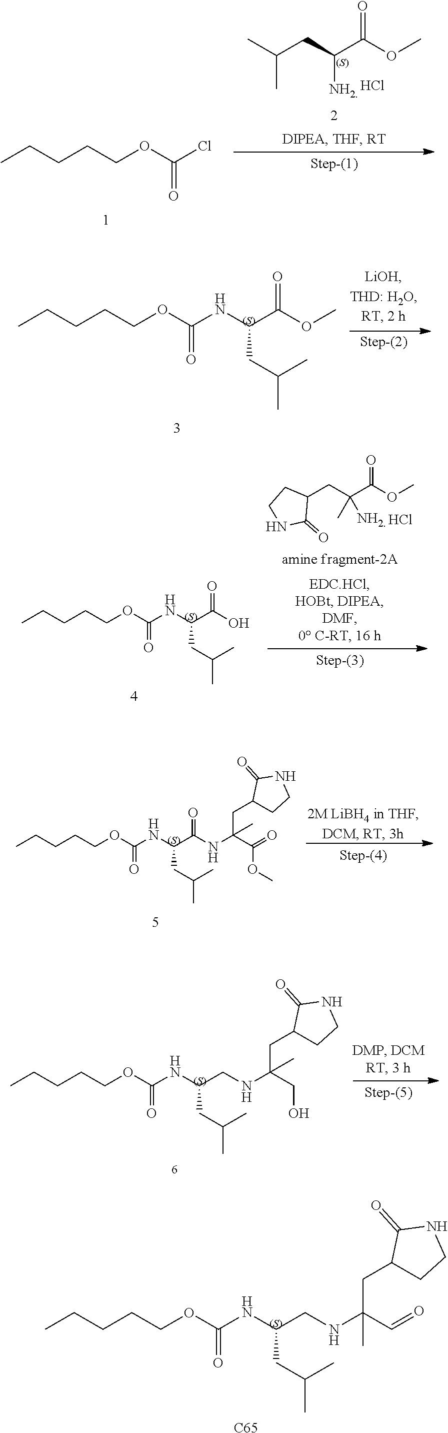

### Methyl ((pentyloxy) carbonyl)-L-leucinate (3)

To a stirred solution of Pentyl carbonochloridate (1) (3.7 g, 24.56 mmol), methyl L-leucinate hydrochloride (3 g, 20.68 mmol) in THF (30 mL), was added DIPEA (10.4 mL, 62.06 mmol) at 0° C. and stirred at room temperature for 3 h. The progress of the reaction was monitored by TLC and LCMS. Reaction mixture was diluted with ethyl acetate (30 mL) and washed with water (2×30 mL), dried over sodium sulfate, concentrated under reduced pressure to afford methyl ((pentyloxy)carbonyl)-L-leucinate (3). TLC system: 50% Ethyl acetate in Pet ether R_{f}: 0.6 LCMS (ESI): m/z 201.39 [M+H]⁺

### ((Pentyloxy) carbonyl)-L-leucine (4)

To a stirred solution of methyl ((pentyloxy)carbonyl)-L-leucinate (3) (2.5 g, mmol) in THF (30 mL), water (10 mL) was added lithium hydroxide (717 mg, 29.90 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford (((2-methyl-1-phenylpropan-2-yl)oxy)carbonyl)-L-leucine (4). TLC system: 100% EtOAc Rf: 0.1 LCMS (ESI): m/z 330.50 [M+Na]

### Methyl 2-methyl-2-((S)-4-methyl-2-(((pentyloxy) carbonyl) amino) pentanamido)-3-(2-oxopyrrolidin-3-yl) propanoate (5)

To a stirred solution of ((pentyloxy)carbonyl)-L-leucine (4) (400 mg, 1.632 mmol) DMF (5 mL) added EDC.HCl (467 mg, 2.44 mmol), HOBt (330 mg, 2.44 mmol), DIPEA (0.87 mL, 4.89 mmol) and methyl 2-amino-2-methyl-3-(2-oxopyrrolidin-3-yl)propanoate hydrochloride (amine fragment-2A) (391 mg, 1.95 mmol) at 0° C. simultaneously and stirred at room temperature for 16 h. Reaction mixture was diluted with ice water (20 mL), extracted with ethyl acetate (2×20 mL), dried over sodium sulfate and evaporated under reduced pressure. The crude residue was purified by combi-flash NP, compound eluted at 4% methanol in dichloromethane to afford methyl 2-methyl-2-((S)-4-methyl-2-(((pentyloxy) carbonyl) amino) pentanamido)-3-(2-oxopyrrolidin-3-yl) propanoate (5). TLC system: 5% MeOH in DCM Rf: 0.4 LCMS (ESI): m/z 524.7 [M+H]⁺

### Pentyl ((2S)-1-((1-hydroxy-2-methyl-3-(2-oxopyrrolidin-3-yl) propan-2-yl) amino)-4-methyl-1-oxopentan-2-yl) carbamate (6)

To a stirred solution of methyl 2-methyl-2-((S)-4-methyl-2-(((pentyloxy)carbonyl)amino) pentanamido)-3-(2-oxopyrrolidin-3-yl)propanoate (5) (400 mg, 0.93 mmol) in DCM (5 mL) was added 2M LiBH₄ in THF (0.9 mL, 1.86 mmol) at 0° C. and the reaction mixture stirred for 2 h at 0° C. The progress of the reaction was monitored by TLC and LCMS. Reaction mixture was quenched with sat. ammonium chloride solution (20 mL) and extracted with DCM (2×15 mL). Organic layer was washed with brine solution (20 mL), dried over Na₂SO₄ and concentrated to afford pentyl ((2S)-1-((1-hydroxy-2-methyl-3-(2-oxopyrrolidin-3-yl) propan-2-yl) amino)-4-methyl-1-oxopentan-2-yl) carbamate (6). TLC system: 10% MeOH in DCM Rf: 0.3 LCMS (ESI): m/z 400.20 [M+H]⁺

### Pentyl ((2S)-4-methyl-1-((2-methyl-1-oxo-3-(2-oxopyrrolidin-3-yl) propan-2-yl) amino)-1-oxopentan-2-yl) carbamate (Compound C65)

To a stirred solution of pentyl ((2S)-1-((1-hydroxy-2-methyl-3-(2-oxopyrrolidin-3-yl)propan-2-yl) amino)-4-methyl-1-oxopentan-2-yl)carbamate (6) (200 mg, 0.50 mmol) was dissolved in dichloromethane (5 mL) was added Dess-Martin periodinane (630 mg, 1.48 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford pentyl ((2S)-4-methyl-1-((2-methyl-1-oxo-3-(2-oxopyrrolidin-3-yl) propan-2-yl) amino)-1-oxopentan-2-yl) carbamate (Compound C65). TLC system: 10% MeOH in DCM Rf: 0.4 LCMS (ESI): m/z 398.2 (M+H)⁺

### Example 76: Synthesis of Compounds C77 and C91

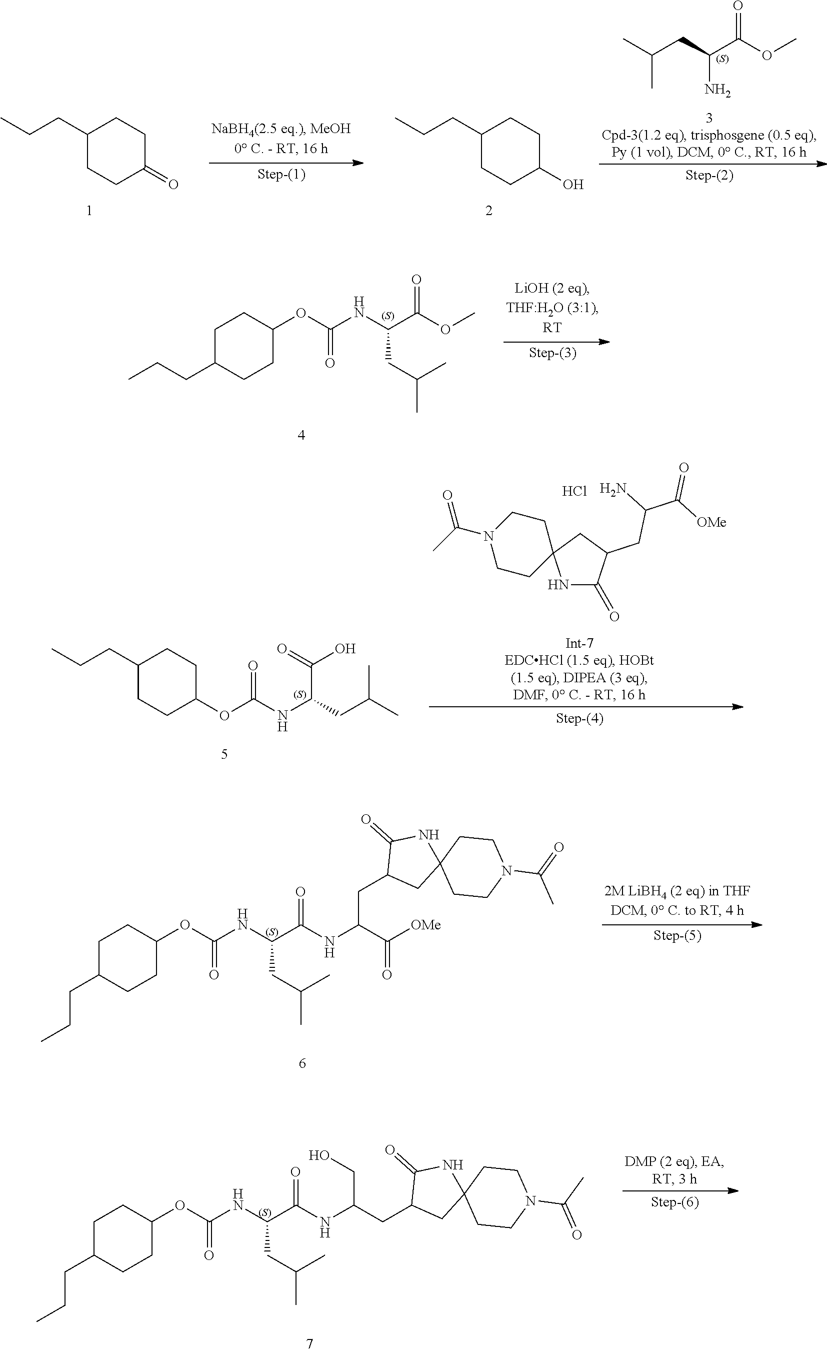

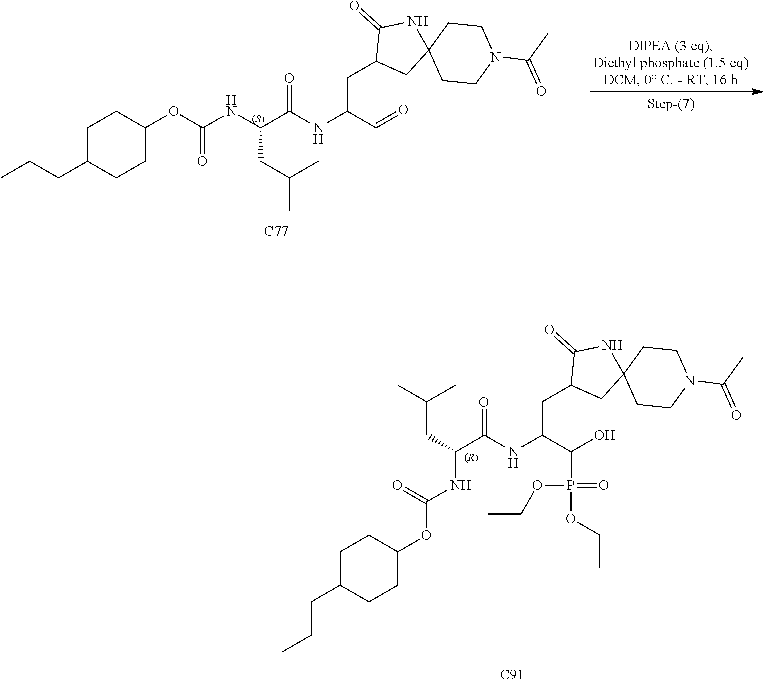

### 4-propylcyclohexan-1-ol (2)

To a stirred solution of 4-propylcyclohexan-1-one (1) (500 mg, 3.57 mmol) in methanol (10 mL) was added sodium borohydride (337 mg, 8.92 mmol) at 0° C. and the reaction mixture stirred for 16 h at RT. The progress of the reaction was monitored by TLC and LCMS. After 16 h, reaction mixture was quenched with water (20 mL) and extracted with ethyl acetate (2×30 mL). Organic layer was washed with brine solution (20 mL), and combined organic layer was dried over Na₂SO₄ and concentrated to afford 4-propylcyclohexan-1-ol (2). TLC system: 30% Ethyl acetate/pet-ether R_{f} 0.2 LCMS (ESI): m/z 124.87 [M−OH]⁺

### Methyl (((4-propylcyclohexyl)oxy)carbonyl)-L-leucinate (4)

To a stirred solution of 4-propylcyclohexan-1-ol (2) (200 mg, 1.418 mmol), methyl L-leucinate (3) (308 mg, 2.1 mmol) in DCM (10 mL) was added pyridine (0.2 mL, 1 vol) followed by triphosgene (209 mg, 0.7 mmol) at 0° C. and stirred at room temperature for 16 h. The progress of the reaction was monitored by TLC and LCMS. Reaction mixture was quenched with ice water (20 mL), extracted with DCM (2×20 mL). The combined organic layer was dried over sodium sulfate, filtered and evaporated under reduced pressure. The crude residue was purified by combi-flash, compound eluted at 8% ethyl acetate in pet ether to afford methyl (((4-propylcyclohexyl)oxy)carbonyl)-L-leucinate (4). TLC system: 20% Ethyl acetate in Pet ether R_{f}: 0.4 LCMS (ESI): m/z 314.51 [M+H]⁺

### (((4-propylcyclohexyl)oxy)carbonyl)-L-leucine (5)

To a stirred solution of methyl (((4-propylcyclohexyl)oxy)carbonyl)-L-leucinate (4) (500 mg, 1.597 mmol) in THF (3 mL), water (1 mL) was added lithium hydroxide (115 mg, 4.8 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×40 mL), dried over sodium sulfate, concentrated under reduced pressure to afford (((4-propylcyclohexyl)oxy)carbonyl)-L-leucine (5). TLC system: 10% MeOH in DCM Rf: 0.2 LCMS (ESI): m/z 300.4 (M+H)⁺

### Methyl 3-(8-acetyl-2-oxo-1,8-diazaspiro[4.5]decan-3-yl)-2-((S)-4-methyl-2-((((4-propylcyclohexyl)oxy)carbonyl)amino)pentanamido)propanoate (6)

To a stirred solution of (((4-propylcyclohexyl)oxy)carbonyl)-L-leucine (5) (700 mg, 2.34 mmol) in DMF (15 mL) at 0° C. was added EDC.HCl (670 mg, 3.51 mmol), HOBT (473 mg, 3.51 mmol), DIPEA (1.2 mL, 7.02 mmol) and methyl 3-(8-acetyl-2-oxo-1,8-diazaspiro[4.5]decan-3-yl)-2-aminopropanoate hydrochloride (Int-7) (974 mg, 1.2 mmol) simultaneously and stirred at room temperature for 16 h. The progress of the reaction was monitored by TLC and LCMS. After 16 h, reaction mixture was quenched with ice water (50 mL), extracted with ethyl acetate (2×100 mL), the combined organic layer was dried over sodium sulfate and evaporated under reduced pressure. The crude residue was purified by silica gel column by eluting with 60% ethyl acetate in pet ether to afford methyl 3-(8-acetyl-2-oxo-1,8-diazaspiro[4.5]decan-3-yl)-2-((S)-4-methyl-2-((((4-propylcyclohexyl)oxy)carbonyl)amino)pentanamido)propanoate (6). TLC system: 5% Methanol in DCM R_{f}: 0.65 LCMS (ESI): m/z 575.91 (M+H)⁺

### Methyl 3-(8-acetyl-2-oxo-1,8-diazaspiro[4.5]decan-3-yl)-2-((S)-4-methyl-2-((((4-propylcyclohexyl)oxy)carbonyl)amino)pentanamido)propanoate (7)

To a stirred solution of methyl 3-(8-acetyl-2-oxo-1,8-diazaspiro[4.5]decan-3-yl)-2-((S)-4-methyl-2-((((4-propylcyclohexyl)oxy)carbonyl)amino)pentanamido)propanoate (6) (350 mg, 0.63 mmol) in DCM (3 mL) was added 2M LiBH₄ in THF (0.63 mL, 1.27 mmol) at 0° C. and the reaction mixture stirred for 2 h at RT. The progress of the reaction was monitored by TLC and LCMS. After 4 h, reaction mixture was quenched with water (20 mL) and extracted with DCM (2×30 mL). Organic layer was washed with brine solution (30 mL), and combined organic layer was dried over Na₂SO₄ and concentrated to afford Methyl 3-(8-acetyl-2-oxo-1,8-diazaspiro[4.5]decan-3-yl)-2-((S)-4-methyl-2-((((4-propylcyclohexyl)oxy)carbonyl)amino) pentanamido)propanoate (7). TLC system: 5% MeOH in DCM R_{f} 0.2 LCMS (ESI): m/z 551.4 (M+H)⁺

### 4-Propylcyclohexyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C77)

To a stirred solution of methyl 3-(8-acetyl-2-oxo-1,8-diazaspiro[4.5]decan-3-yl)-2-((S)-4-methyl-2-((((4-propylcyclohexyl)oxy)carbonyl)amino)pentanamido)propanoate (7) (290 mg, 0.52 mmol) in ethyl acetate (4 mL) was added Dess-Martin periodinane (670 mg, 1.58 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 4-propylcyclohexyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C77). TLC system: 5% Methanol in DCM R_{f}: 0.4 LCMS (ESI): m/z 549.3 (M+H)⁺

### 4-Propylcyclohexyl ((2R)-1-((3-(8-acetyl-2-oxo-1,8-diazaspiro[4.5]decan-3-yl)-1-(diethoxyphosphoryl)-1-hydroxypropan-2-yl)amino)-4-methyl-1-oxopentan-2-yl)carbamate (Compound C91)

To a stirred solution of 4-propylcyclohexyl ((2S)-1-((1-(8-acetyl-2-oxo-1,8-diazaspiro[4.5]decan-3-yl)-3-oxopropan-2-yl)amino)-4-methyl-1-oxopentan-2-yl)carbamate (Compound C77) (450 mg, 0.821 mmol) was dissolved in DCM (2 mL) was added DIPEA (0.34 mL, 2.46 mmol) and diethyl phosphite (0.42 mL, 2.46 mmol) at 0° C. and stirred at RT for 16 h. Reaction mixture was quenched with ice water (10 mL) extracted with DCM (3×20 mL). Organic layer was dried over anhydrous Na₂SO₄, filtered and purified by prep HPLC to afford pure diethyl (3-(8-acetyl-2-oxo-1,8-diazaspiro[4.5]decan-3-yl)-2-((S)-3-cyclohexyl-2-(1H-indole-2-carboxamido)propanamido)-1-hydroxypropyl)phosphonate (Compound C91). TLC system: 100% EtOAc in pet ether Rf: 0.25 LCMS (ESI): m/z 687.3 (M+H)⁺

### Example 77: Synthesis of Compound C83

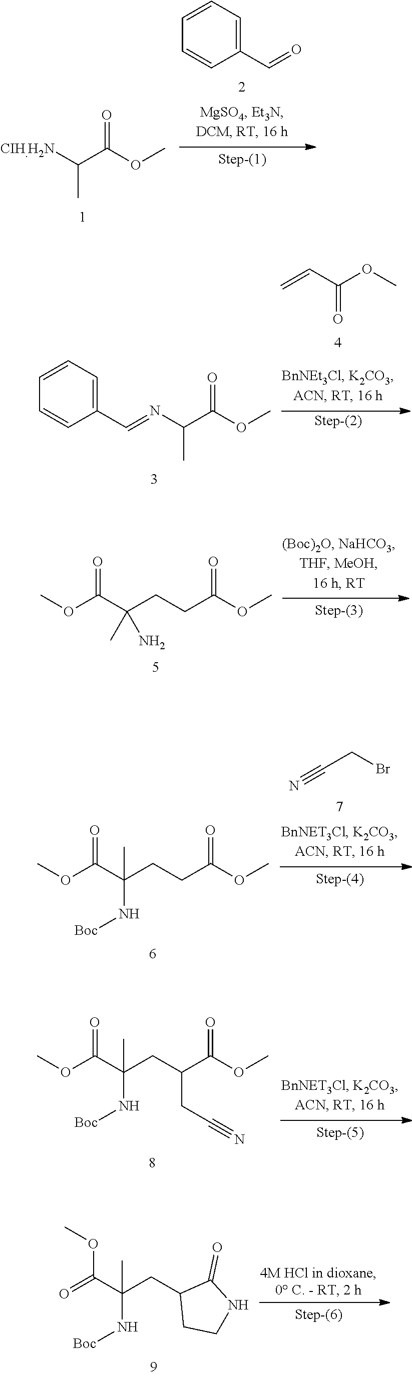

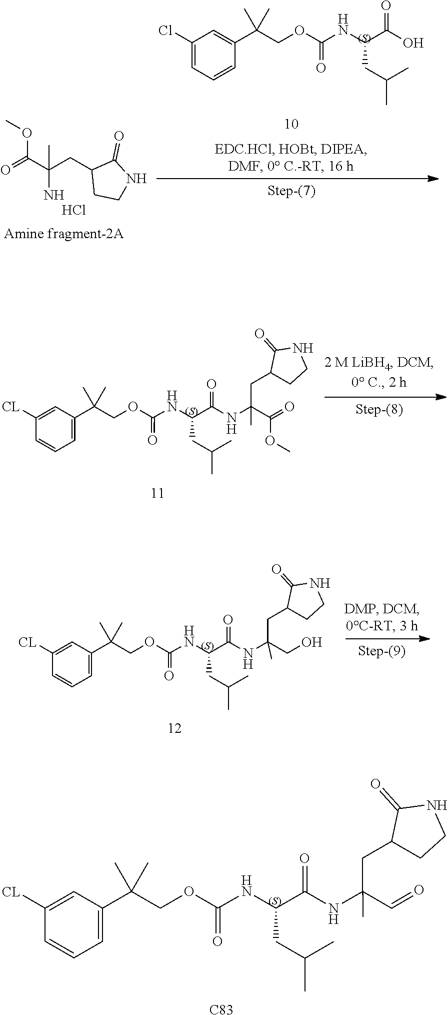

### Methyl (E)-2-(benzylideneamino) propanoate (3)

To a stirred solution of methyl alaninate hydrochloride (1) (15 g, 108.30 mmol), benzaldehyde (11.4 g, 108.30 mmol) in DCM (100 mL) was added triethylamine (18.2 mL, 129.96 mmol) followed by magnesium sulfate (9.1 g, 75.81 mmol) at 0° C. and stirred at RT for 16 h. The progress of the reaction was monitored by TLC, Reaction mixture was filtered through celite bed and washed with DCM (50 mL). Filtrate was washed with water (2×50 mL), dried over sodium sulfate, filtered and evaporated under reduced pressure to afford crude methyl (E)-2-(benzylideneamino) propanoate (3) which was used in the next step. TLC system: 10% Ethyl acetate in Pet ether Rf: 0.5 LCMS (ESI): m/z 192.38 [M+H]⁺

### Dimethyl 2-amino-2-methylpentanedioate (5)

To a stirred solution of crude methyl (E)-2-(benzylideneamino) propanoate (3) (15 g, 78.53 mmol) in acetonitrile (100 mL) was added potassium carbonate (32.5 g, 235.60 mmol), benzyl triethyl ammonium chloride (3.5 g, 15.70 mmol) at 0° C. followed by methyl acrylate (10 g, 117.80 mmol) simultaneously and stirred for 16 h. The progress of the reaction was monitored by TLC. Reaction mixture was quenched with sat. ammonium chloride and extracted with EtOAc (2×50 mL), combined the organic layer and washed with brine solution (2×60 mL), dried over sodium sulfate, filtered and evaporated under reduced pressure to afford crude, residue was purified by combi-flash, compound eluted at 80% ethyl acetate in pet ether to afford dimethyl 2-amino-2-methylpentanedioate (5). TLC system: 80% Ethyl acetate Rf: 0.2

### Dimethyl 2-((tert-butoxycarbonyl) amino)-2-methylpentanedioate (5)

To a stirred solution of dimethyl 2-amino-2-methylpentanedioate (5) (7.5 g, 39.68 mmol), in methanol (75 mL), THF (75 mL) was added NaHCO₃ (6.6 g, 78.57 mmol) followed by boc anhydride (12.9 mL, 59.52 mmol) at 0° C. and stirred at room temperature for 16 h. The progress of the reaction was monitored by TLC and LCMS. Reaction mixture was quenched with ice water (30 mL), extracted with ethyl acetate (2×50 mL). The combined organic layer was dried over sodium sulfate, filtered and evaporated under reduced pressure. The crude residue was purified by combi-flash, compound eluted at 5% ethyl acetate in pet ether to afford dimethyl 2-((tert-butoxycarbonyl) amino)-2-methylpentanedioate (6). TLC system: 30% Ethyl acetate in Pet ether R_{f}: 0.6 LCMS (ESI): m/z 312.27 [M+Na]⁺

### Dimethyl 2-((tert-butoxycarbonyl) amino)-4-(cyanomethyl)-2-methylpentanedioate (6)

To a stirred solution of dimethyl 2-((tert-butoxycarbonyl) amino)-2-methylpentanedioate (5) (7.5 g, 25.95 mmol) in THF (70 mL)), was added 1M LiHMDS in THF (57 mL, 29.95 mmol) at −78° C. and stirred for 1 h then bromoacetonitrile (2.16 g, 31.14 mmol) was added with dropwide and stirring was continued another 3 h at same temperature. The progress of the reaction was monitored by TLC and LCMS. Reaction mixture was quenched with methanol (10 mL) and acetic acid (5 mL) and stirred at RT for 1 h then extracted with ethyl acetate (2×50 mL), dried over sodium sulfate, concentrated under reduced pressure to afford crude dimethyl 2-((tert-butoxycarbonyl)amino)-4-(cyanomethyl)-2-methylpentanedioate (8) which was used directly in the next step. TLC system: 5% MeOH in DCM R_{f}: 0.5

### Methyl 2-((tert-butoxycarbonyl) amino)-2-methyl-3-(2-oxopyrrolidin-3-yl) propanoate (6)

A solution of COC1₂.6H₂O (10.3 g, 45.73 mmol) and dimethyl 2-((tert-butoxycarbonyl) amino)-4-(cyanomethyl)-2-methylpentanedioate (8) (7.3 g, 22.25 mmol) in MeOH (70 mL) was stirred vigorously and cooled to 0° C. the added NaBH₄ (3.3 g, 86.84 mmol) with portion wise over 30 min. The reaction was stirred at RT for 24 h. The progress of the reaction was monitored by TLC. Reaction mixture was concentrated to remove methanol and quenched with 1N HCl (50 mL) then formed solids were filtered through celite bed and washed with ethyl acetate (100 mL). Two layers were separated and dried over sodium sulfate, filtered, and concentrated to afford crude, this residue was purified by column chromatography to afford methyl 2-((tert-butoxycarbonyl) amino)-2-methyl-3-(2-oxopyrrolidin-3-yl) propanoate (9). TLC system: 10% MeOH in DCM R_{f}: 0.5 LCMS (ESI): m/z 301.10 [M+H]⁺

### Methyl 2-amino-2-methyl-3-(2-oxopyrrolidin-3-yl) propanoate (Amine fragment-2A)

A solution of 4M HCl in dioxane (5 mL) was added to a solution of methyl 2-((tert-butoxycarbonyl) amino)-2-methyl-3-(2-oxopyrrolidin-3-yl) propanoate (9) (0.5 g, 1.66 mmol) in 1,4 dioxane (5 mL) at 0° C. The mixture was stirred for 2 h and then concentrated to afforded crude methyl 2-amino-2-methyl-3-(2-oxopyrrolidin-3-yl) propanoate. HCl salt (Amine fragment-2A) which was used directly in the next step. TLC system: 10% MeOH in DCM R_{f}: 0.1 LCMS (ESI): m/z 201.39 [M+H]⁺

### Methyl 2-((S)-2-(((2-(3-chlorophenyl)-2-methylpropoxy) carbonyl) amino)-4-methylpentanamido)-2-methyl-3-(2-oxopyrrolidin-3-yl) propanoate (11)

To a stirred solution of ((2-(3-chlorophenyl)-2-methylpropoxy) carbonyl)-L-leucine (10) (600 mg, 1.910 mmol) DMF (10 mL) added EDC.HCl (547 mg, 2.82 mmol), HOBt (386 mg, 2.85 mmol), DIPEA (1 mL, 5.73 mmol) and methyl 2-amino-2-methyl-3-(2-oxopyrrolidin-3-yl)propanoate hydrochloride (amine fragment-2A) (458 mg, 2.29 mmol) at 0° C. simultaneously and stirred at room temperature for 16 h. Reaction mixture was diluted with ice water (30 mL), extracted with ethyl acetate (2×20 mL), dried over sodium sulfate and evaporated under reduced pressure. The crude residue was purified by combi-flash NP, compound eluted at 4% methanol in dichloromethane to afford methyl 2-((S)-2-(((2-(3-chlorophenyl)-2-methylpropoxy) carbonyl) amino)-4-methylpentanamido)-2-methyl-3-(2-oxopyrrolidin-3-yl) propanoate (11). TLC system: 5% MeOH in DCM Rf: 0.4 LCMS (ESI): m/z 524.7 [M+H]⁺

### 2-(3-Chlorophenyl)-2-methylpropyl ((2S)-1-((1-hydroxy-2-methyl-3-(2-oxopyrrolidin-3-yl) propan-2-yl) amino)-4-methyl-1-oxopentan-2-yl) carbamate (12)

To a stirred solution of methyl 2-((S)-2-(((2-(3-chlorophenyl)-2-methylpropoxy)carbonyl) amino)-4-methylpentanamido)-2-methyl-3-(2-oxopyrrolidin-3-yl)propanoate (11) (550 mg, 1.049 mmol) in DCM (5 mL) was added 2M LiBH₄ in THF (1 mL, 2.09 mmol) at 0° C. and the reaction mixture stirred for 2 h at 0° C. The progress of the reaction was monitored by TLC and LCMS. Reaction mixture was quenched with sat. Ammonium chloride solution (20 mL) and extracted with DCM (2×15 mL). Organic layer was washed with brine solution (20 mL), dried over Na₂SO₄ and concentrated to afford 2-(3-chlorophenyl)-2-methylpropyl ((2S)-1-((1-hydroxy-2-methyl-3-(2-oxopyrrolidin-3-yl)propan-2-yl)amino)-4-methyl-1-oxopentan-2-yl)carbamate (12). TLC system: 10% MeOH in DCM Rf: 0.3 LCMS (ESI): m/z 496.7 [M+H]⁺

### 2-(3-Chlorophenyl)-2-methylpropyl ((2S)-4-methyl-1-((2-methyl-1-oxo-3-(2-oxopyrrolidin-3-yl) propan-2-yl) amino)-1-oxopentan-2-yl) carbamate (Compound C83)

To a stirred solution of 2-(3-chlorophenyl)-2-methylpropyl ((2S)-1-((1-hydroxy-2-methyl-3-(2-oxopyrrolidin-3-yl)propan-2-yl)amino)-4-methyl-1-oxopentan-2-yl)carbamate (12) (160 mg, 0.32 mmol) was dissolved in dichloromethane (5 mL) was added Dess-Martin periodinane (411 mg, 0.96 mmol) at 0° C. and stirred at RT for 2 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford 2-(3-chlorophenyl)-2-methylpropyl ((2S)-4-methyl-1-((2-methyl-1-oxo-3-(2-oxopyrrolidin-3-yl) propan-2-yl) amino)-1-oxopentan-2-yl) carbamate (Compound C83). TLC system: 5% MeOH in DCM Rf: 0.4 LCMS (ESI): m/z 494.2 (M+H)⁺

### Example 78: Synthesis of Compounds C101 and C92

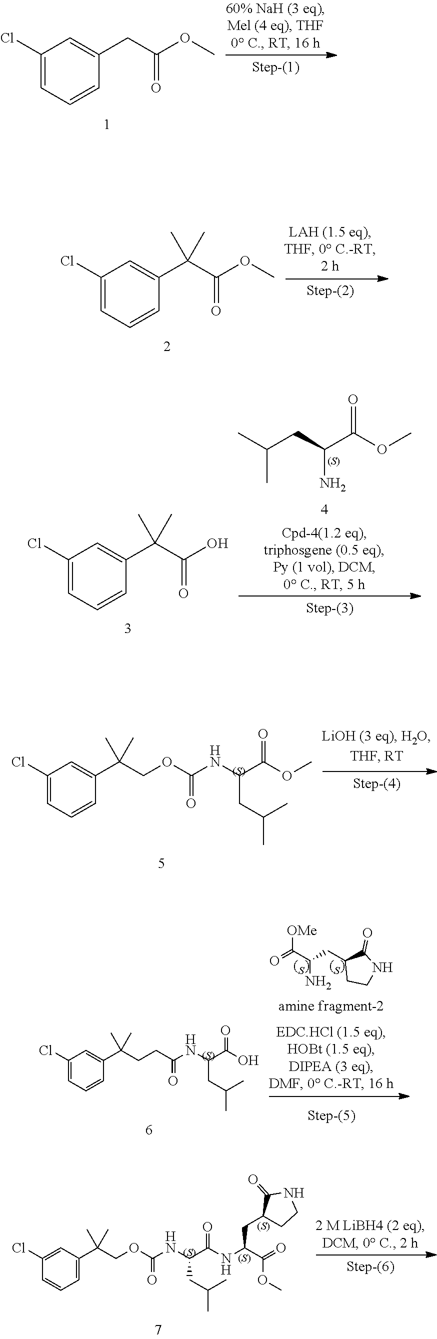

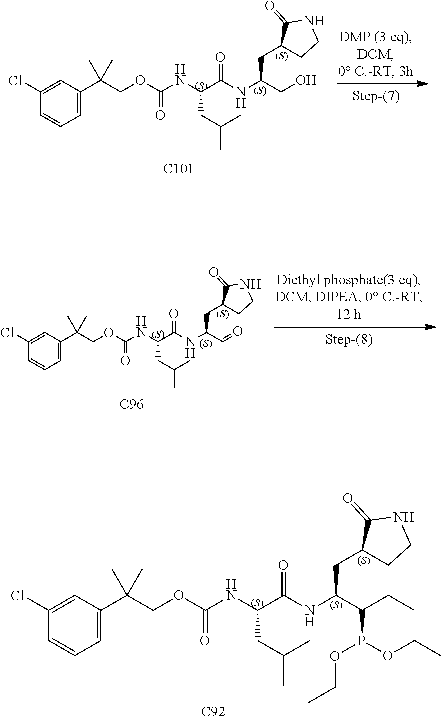

### Methyl 2-(3-chlorophenyl)-2-methylpropanoate (2)

To a stirred solution of methyl 2-(3-chlorophenyl)acetate (1) (10 g, 54.17 mmol) in THF (100 mL) was added 60% NaH (6.5 g, 162.51 mmol) at 0° C. and stirred for 15 min then added Mel (13.5 mL, 216.68 mmol) at same temperature and allowed to RT for 16 h. The progress of the reaction was monitored by TLC, Reaction mixture was quenched with sat. ammonium chloride and extracted with EtOAc (2×100 mL), combined the organic layer and washed with brine solution (100 mL), dried over sodium sulfate, filtered and evaporated under reduced pressure to afford crude, residue was purified by combi-flash, compound eluted at 5% ethyl acetate in pet ether to afford methyl 2-(3-chlorophenyl)-2-methylpropanoate (2). TLC system: 10% Ethyl acetate in Pet ether Rf: 0.3 LCMS (ESI): m/z 213.31 [M+H]⁺

### 2-(3-Chlorophenyl)-2-methylpropan-1-ol (3)

To a stirred solution of methyl 2-(3-chlorophenyl)-2-methylpropanoate (6 g, 28.30 mmol), in THF (80 mL) was added slowly 2.4 M LAH in THF (11.7 mL, 28.30 mmol) at −50° C. and stirred for 1 h. The progress of the reaction was monitored by TLC. Reaction mixture was quenched with sat. ammonium chloride and extracted with EtOAc (2×50 mL), combined the organic layer and washed with brine solution (60 mL), dried over sodium sulfate, filtered and evaporated under reduced pressure to afford crude, residue was purified by combi-flash, compound eluted at 10% ethyl acetate in pet ether to afford 2-(3-chlorophenyl)-2-methylpropan-1-ol (3). TLC system: 30% Ethyl acetate LCMS (ESI): m/z=167.4 [M−OH]⁺

### Methyl ((2-(3-chlorophenyl)-2-methylpropoxy) carbonyl)-L-leucinate (5)

To a stirred solution of 2-(3-chlorophenyl)-2-methylpropan-1-ol (3) (2.8 g, 15.16 mmol), methyl L-leucinate (4) (2.64 g, 18.19 mmol) in DCM (40 mL) was added pyridine (3 mL, 1 vol) followed by triphosgene (2.24 g, 7.58 mmol) at 0° C. and stirred at room temperature for 3 h. The progress of the reaction was monitored by TLC and LCMS. Reaction mixture was quenched with ice water (30 mL), extracted with DCM (2×30 mL). The combined organic layer was dried over sodium sulfate, filtered and evaporated under reduced pressure. The crude residue was purified by combi-flash, compound eluted at 8% ethyl acetate in pet ether to afford methyl ((2-(3-chlorophenyl)-2-methylpropoxy) carbonyl)-leucinate (5). TLC system: 30% Ethyl acetate in Pet ether R_{f}: 0.5 LCMS (ESI): m/z 356.31 [M+H]⁺

### ((2-(3-Chlorophenyl)-2-methylpropoxy) carbonyl)-L-leucine (6)

To a stirred solution of methyl ((2-(3-chlorophenyl)-2-methylpropoxy) carbonyl)-leucinate (5) (2.3 g, 6.47 mmol) in THF (10 mL) and water (5 mL), was added lithium hydroxide (800 mg, 19.43 mmol) at RT and stirred at room temperature for 3 h. The progress of the reaction was monitored by TLC and LCMS. Reaction mixture completely distilled under reduced pressure and diluted with and washed with diethyl ether (20 mL), aq layer was acidified with aq. 1N HCl solution up to pH˜4, and extracted with dichloromethane (2×30 mL), dried over sodium sulfate, concentrated under reduced pressure to afford ((2-(3-chlorophenyl)-2-methylpropoxy) carbonyl)-L-leucine (6). TLC system: 10% MeOH in DCM R_{f}: 0.1 LCMS (ESI): m/z 342.34 [M+H]⁺

### Methyl (S)-2-((S)-2-(((2-(3-chlorophenyl)-2-methylpropoxy) carbonyl) amino)-4-methylpentanamido)-3-((S)-2-oxopyrrolidin-3-yl) propanoate (7)

To a stirred solution of ((2-(3-chlorophenyl)-2-methylpropoxy) carbonyl)-L-leucine (6) (10 g, 29.32 mmol) in DMF (100 mL) added EDC.HCl (8.401 g, 43.98 mmol), HOBt (5.76 g, 43.98 mmol), DIPEA (15.2 mL, 87.9 mmol) and methyl (S)-2-amino-3-((S)-2-oxopyrrolidin-3-yl)propanoate hydrochloride (amine fragment-2) (6.545 g, 35.19 mmol) at 0° C. simultaneously and stirred at room temperature for 16 h. Reaction mixture was diluted with ice water (100 mL), extracted with ethyl acetate (2×50 mL), dried over sodium sulfate and evaporated under reduced pressure. The crude residue was purified by combi-flash NP, compound eluted at 4% methanol in dichloromethane to afford methyl (S)-2-((S)-2-(((2-(3-chlorophenyl)-2-methylpropoxy) carbonyl)amino)-4-methylpentanamido)-3-((S)-2-oxopyrrolidin-3-yl)propanoate (7). TLC system: 100% Ethyl acetate Rf: 0.4 LCMS (ESI): m/z 510.55 [M+H]⁺

### 2-(3-Chlorophenyl)-2-methylpropyl ((S)-1-(((S)-1-hydroxy-3-((S)-2-oxopyrrolidin-3-yl)propan-2-yl)amino)-4-methyl-1-oxopentan-2-yl)carbamate (Compound C101)

To a stirred solution of methyl (S)-2-((S)-2-(((2-(3-chlorophenyl)-2-methylpropoxy) carbonyl) amino)-4-methylpentanamido)-3-((S)-2-oxopyrrolidin-3-yl)propanoate (7) (2×7 g, 13.75 mmol) in DCM (70 mL) was added 2M LiBH₄ in THF (20 mL, 41.25 mmol) at 0° C. and the reaction mixture stirred for 2 h at 0° C. The progress of the reaction was monitored by TLC and LCMS. Reaction mixture was quenched with sat. Ammonium chloride solution (40 mL) and extracted with DCM (2×30 mL). Organic layer was washed with brine solution (30 mL), dried over Na₂SO₄ and concentrated to afford mixture of cpd-8 (10 g, 20.75 mmol, 75.58% yield). The mixture was purified by SFC to afford 2-(3-Chlorophenyl)-2-methylpropyl ((S)-1-(((S)-1-hydroxy-3-((S)-2-oxopyrrolidin-3-yl) propan-2-yl) amino)-4-methyl-1-oxopentan-2-yl)carbamate (Compound C101). TLC system: 100% Ethyl acetate Rf: 0.2

### 2-(3-Chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C96)

To a stirred solution of 2-methyl-1-phenylpropan-2-yl ((S)-1-(((S)-1-hydroxy-3-((S)-2-oxopyrrolidin-3-yl)propan-2-yl)amino)-4-methyl-1-oxopentan-2-yl)carbamate (8) (1 g, 2.079 mmol) was dissolved in dichloromethane (10 mL) was added Dess-Martin periodinane (2.644 g, 6.237 mmol) at 0° C. and stirred at RT for 2 h. Reaction mixture was diluted with DCM (15 mL) followed by sat. Hypo solution (3×15 mL), followed by sat. NaHCO₃ solution (3×15 mL). Organic layer was dried over anhydrous Na₂SO₄, filtered and concentrated to get crude compound. The crude compound was purified by normal phase silica gel purification 10% MeOH/DCM product was eluted as a 2-(3-chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C96). TLC system: 10% MeOH in DCM Rf: 0.4 LCMS (ESI): m/z 480.2 (M+H)⁺

### 2-(3-Chlorophenyl)-2-methylpropyl ((2S)-1-(((2S)-1-(diethoxyphosphaneyl)-1-hydroxy-3-((S)-2-oxopyrrolidin-3-yl)propan-2-yl)amino)-4-methyl-1-oxopentan-2-yl)carbamate (Compound C92)

To a stirred solution of 2-(3-chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C96) in DCM (5 mL) added Diethyl phosphate (138 mg, 1.002 mmol), DIPEA (0.18 mL, 1.002 mmol) and at 0° C. simultaneously and stirred at room temperature for 16 h. Reaction mixture was diluted with ice water (100 mL), extracted with 10% methanol/DCM (3×20 mL), dried over sodium sulfate and evaporated under reduced pressure. The crude residue was purified by Prep-HPLC to afford 2-(3-chlorophenyl)-2-methylpropyl ((2S)-1-(((2S)-1-(diethoxyphosphaneyl)-1-hydroxy-3-((S)-2-oxopyrrolidin-3-yl)propan-2-yl)amino)-4-methyl-1-oxopentan-2-yl)carbamate (Compound C92). TLC system: 100% Ethyl acetate Rf: 0.3 LCMS (ESI): m/z 618.2 [M+H]⁺

### Example 79: Synthesis of Compound C102

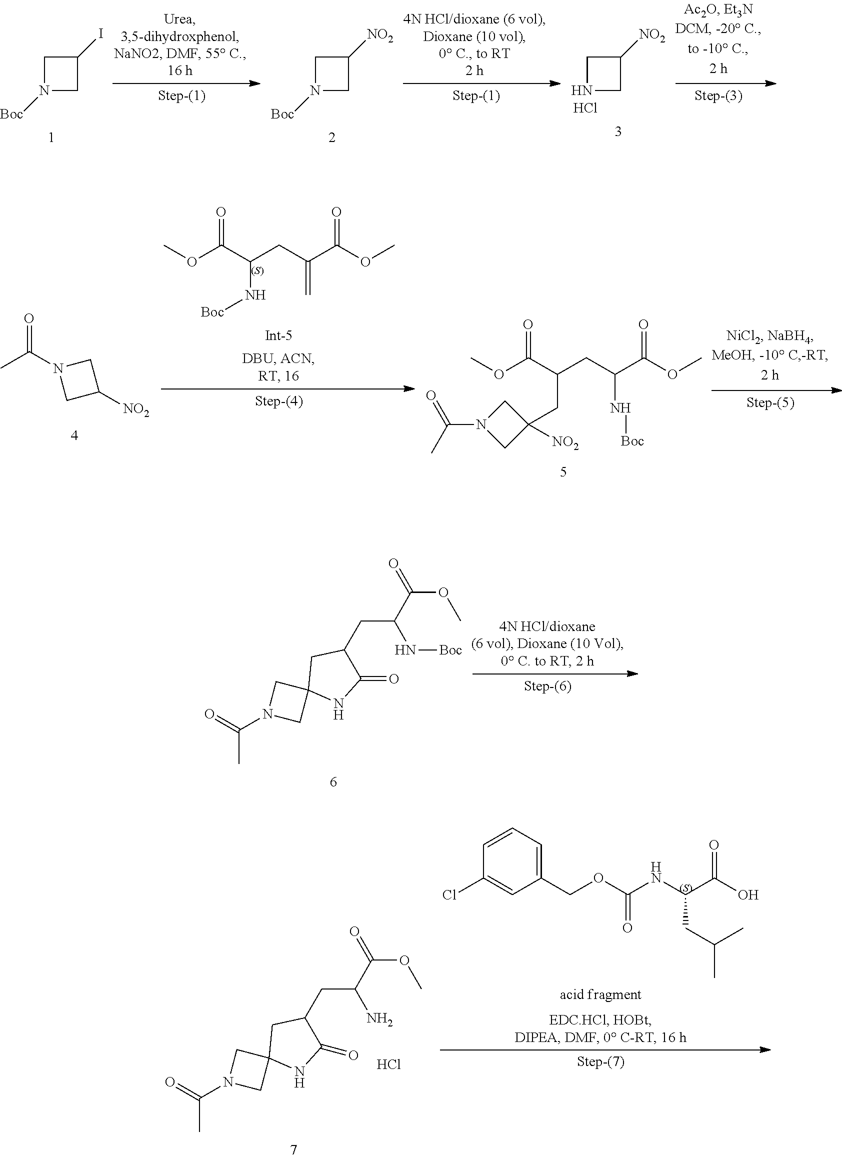

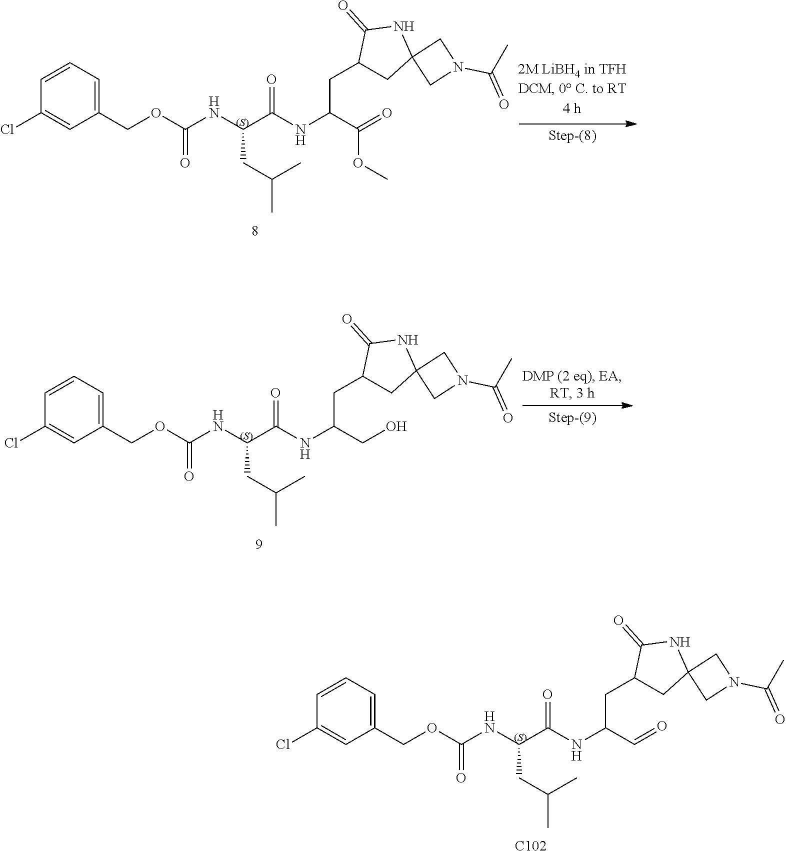

### Tert-butyl 3-nitroazetidine-1-carboxylate (2)

To a stirred solution of tert-butyl 3-iodoazetidine-1-carboxylate (1) (20 g, 70.671 mmol) in DMSO (100 mL) was added phloroglucinol (10.225 g, 148.409 mmol), followed by sodium nitrite (9.79 g, 77.738 mmol) at room temperature and stirred at 45° C. for 16 h. The progress of the reaction was monitored by TLC. The reaction mixture was quenched with water (250 mL) and extracted with diethyl ether (3×100 mL), combined organic layers were washed with water (2×50 ml), brine solution (50 mL), dried over sodium sulfate and evaporated under reduced pressure. The crude residue was purified by normal phase chromatography to afford tert-butyl 3-nitroazetidine-1-carboxylate (2). TLC system: 50% EtOAc/Pet ether R_{f}: 0.45

### 3-nitroazetidine hydrochloride (3)

To a stirred solution of tert-butyl 3-nitroazetidine-1-carboxylate (2) (3.5 g, 17.326 mmol) in 1,4-dioxane (5 mL) was added 4M HCl in 1,4-dioxane (5 mL) at 0° C. and stirred at room temperature for 4 h. The progress of the reaction was monitored by TLC and LCMS. After 4 h, the reaction mixture completely distilled under reduced pressure, crude compound was triturated with diethyl ether (2×10 mL) to afford 3-nitroazetidine hydrochloride (3). TLC system: 5% MeOH/DCM R_{f}: 0.1 LCMS (ESI): m/z 102.80 [M+H]⁺

### 1-(3-nitroazetidin-1-yl)ethan-1-one (4)

To a stirred solution of 3-nitroazetidine hydrochloride (3) (3.3 g, 23.913 mmol) was dissolved in DCM (20 mL) was added acetic anhydride (2.2 mL, 23.913 mmol) and triethyl amine (2.7 mL, 35.869 mmol) at 0° C. simultaneously and stirred at RT for 2 h. The progress of the reaction was monitored by TLC and LCMS. Reaction mixture was quenched with ice water (20 mL) extracted with DCM (2×20 mL), organic layers were washed with water (2×10 ml), brine solution (10 mL), dried over sodium sulfate and evaporated under reduced pressure. The crude residue was purified by normal phase chromatography to afford 1-(3-nitroazetidin-1-yl)ethan-1-one. TLC system: 30% EtOAc in pet ether Rf: 0.3 LCMS (ESI): m/z 144.78 [M+H]⁺

### Dimethyl 2-((1-acetyl-3-nitroazetidin-3-yl)methyl)-4-((tert-butoxycarbonyl)amino)pentanedioate (5)

To a stirred solution of 1-(3-nitroazetidin-1-yl)ethan-1-one (4) (1.5 g, 10.416 mmol) in ACN (15 mL) was added dimethyl (S)-2-((tert-butoxycarbonyl)amino)-4-methylenepentanedioate Int-5 dimethyl 2-((1-acetyl-3-nitroazetidin-3-yl)methyl)-4-((tert-butoxycarbonyl)amino)pentanedioate (5) (3.3 g, 11.458 mmol) and DBU (1.9 mL, 12.499 mmol) at 0° C. and stirred at RT for 16 h. The progress of the reaction was monitored by TLC and LCMS. Reaction mixture was evaporated under reduced pressure. The crude residue was purified by normal phase chromatography to afford dimethyl 2-((1-acetyl-4-nitropiperidin-4-yl)methyl)-4-((tert-butoxycarbonyl)amino)pentanedioate (5). TLC system: 5% MeOH/DCM R_{f}: 0.35 LCMS (ESI): m/z 454.36 (M+Na+H)⁺

### Methyl 3-(2-acetyl-6-oxo-2,5-diazaspiro[3.4]octan-7-yl)-2-((tert-butoxycarbonyl)amino)propanoate (6)

To a stirred solution of dimethyl 2-((1-acetyl-3-nitroazetidin-3-yl)methyl)-4-((tert-butoxycarbonyl)amino)pentanedioate (5) (5.2 g, 12.06 mmol) in methanol (100 mL) was added nickel chloride (1.7 g, 13.27 mmol), followed by sodium borohydride (2.29 g, 60.3 mmol) at −10° C. and stirred at RT for 2 h. The progress of the reaction was monitored by TLC and LCMS. The reaction mixture was quenched with water (50 mL) and extracted with DCM (3×150 mL), combined organic layers were washed with water (2×50 ml), brine solution (30 mL), dried over sodium sulfate and evaporated under reduced pressure. The crude residue was purified by normal phase chromatography to afford methyl 3-(2-acetyl-6-oxo-2,5-diazaspiro[3.4]octan-7-yl)-2-((tert-butoxycarbonyl)amino)propanoate (6). TLC system: 10% MeOH/DCM R_{f}: 0.2 LCMS (ESI): m/z 370.2 (M+H)⁺

### Methyl 3-(2-acetyl-6-oxo-2,5-diazaspiro[3.4]octan-7-yl)-2-aminopropanoate (7)

To a stirred solution of methyl 3-(2-acetyl-6-oxo-2,5-diazaspiro[3.4]octan-7-yl)-2-((tert-butoxycarbonyl)amino)propanoate (6) (2 g, 5.42 mmol) in DCM (4 mL) was added 1,4-dioxane.HCl (12 mL) at 0° C. and stirred at RT for 2 h. The progress of the reaction was monitored by TLC. Reaction mixture was evaporated under reduced pressure. The crude residue was trituration with n-pentane to afford methyl 3-(2-acetyl-6-oxo-2,5-diazaspiro[3.4]octan-7-yl)-2-aminopropanoate (7) TLC system: 15% MeOH/DCM R_{f}: 0.1 LCMS (ESI): m/z 270.21 (M+H)⁺

### Methyl 3-(2-acetyl-6-oxo-2,5-diazaspiro[3.4]octan-7-yl)-2-((S)-2-((((3-chlorobenzyl)oxy)carbonyl)amino)-4-methylpentanamido)propanoate (8)

To a stirred solution of (((3-chlorobenzyl)oxy)carbonyl)-L-leucine (1 g, 3.34 mmol) in DMF (12 mL) at 0° C. was added EDC.HCl (0.7 g, 3.67 mmol), HOBT (0.49 g, 3.67 mmol), DIPEA (1.8 mL, 10.02 mmol) and methyl 3-(8-acetyl-2-oxo-1,8-diazaspiro[4.5]decan-3-yl)-2-aminopropanoate hydrochloride (8) (1 g, 3.64 mmol) simultaneously and stirred at room temperature for 16 h. The progress of the reaction was monitored by TLC and LCMS. After 16 h, reaction mixture was quenched with ice water (60 mL), extracted with ethyl acetate (2×100 mL), the combined organic layer was dried over sodium sulfate and evaporated under reduced pressure. The crude residue was purified by silica gel column by eluting with 50% ethyl acetate in pet ether to afford methyl 3-(2-acetyl-6-oxo-2,5-diazaspiro[3.4]octan-7-yl)-2-((S)-2-((((3-chlorobenzyl)oxy)carbonyl)amino)-4-methylpentanamido) propanoate (8). TLC system: 5% Methanol in DCM R_{f}: 0.6 LCMS (ESI): m/z 551.41 (M+H)⁺

### 3-Chlorobenzyl ((2S)-1-((1-(2-acetyl-6-oxo-2,5-diazaspiro[3.4]octan-7-yl)-3-hydroxypropan-2-yl)amino)-4-methyl-1-oxopentan-2-yl)carbamate (9)

To a stirred solution of methyl 3-(2-acetyl-6-oxo-2,5-diazaspiro[3.4]octan-7-yl)-2-((S)-2-((((3-chlorobenzyl)oxy)carbonyl)amino)-4-methylpentanamido) propanoate (9) (600 mg, 0.94 mmol) in DCM (10 mL) was added 2M LiBH₄ in THF (0.9 mL, 1.89 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford 3-chlorobenzyl ((2S)-1-((1-(2-acetyl-6-oxo-2,5-diazaspiro[3.4]octan-7-yl)-3-hydroxypropan-2-yl)amino)-4-methyl-1-oxopentan-2-yl)carbamate (9). TLC system: 5% MeOH in DCM R_{f} 0.3 LCMS (ESI): m/z 523.57 (M+H)⁺

### 3-Chlorobenzyl ((2S)-1-((1-(2-acetyl-6-oxo-2,5-diazaspiro[3.4]octan-7-yl)-3-oxopropan-2-yl)amino)-4-methyl-1-oxopentan-2-yl)carbamate (Compound C102)

To a stirred solution of 3-chlorobenzyl ((2S)-1-((1-(2-acetyl-6-oxo-2,5-diazaspiro[3.4]octan-7-yl)-3-hydroxypropan-2-yl)amino)-4-methyl-1-oxopentan-2-yl)carbamate (9) (100 mg, 0.19 mmol) in ethyl acetate (5 mL) was added Dess-Martin periodinane (478 mg, 0.76 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 3-chlorobenzyl ((2S)-1-((1-(2-acetyl-6-oxo-2,5-diazaspiro[3.4]octan-7-yl)-3-oxopropan-2-yl)amino)-4-methyl-1-oxopentan-2-yl)carbamate (Compound C102). TLC system: 5% Methanol in DCM R_{f}: 0.4 LCMS (ESI): m/z 521.2 (M+H)⁺

### Example 80: Synthesis of Compounds C103 and C111

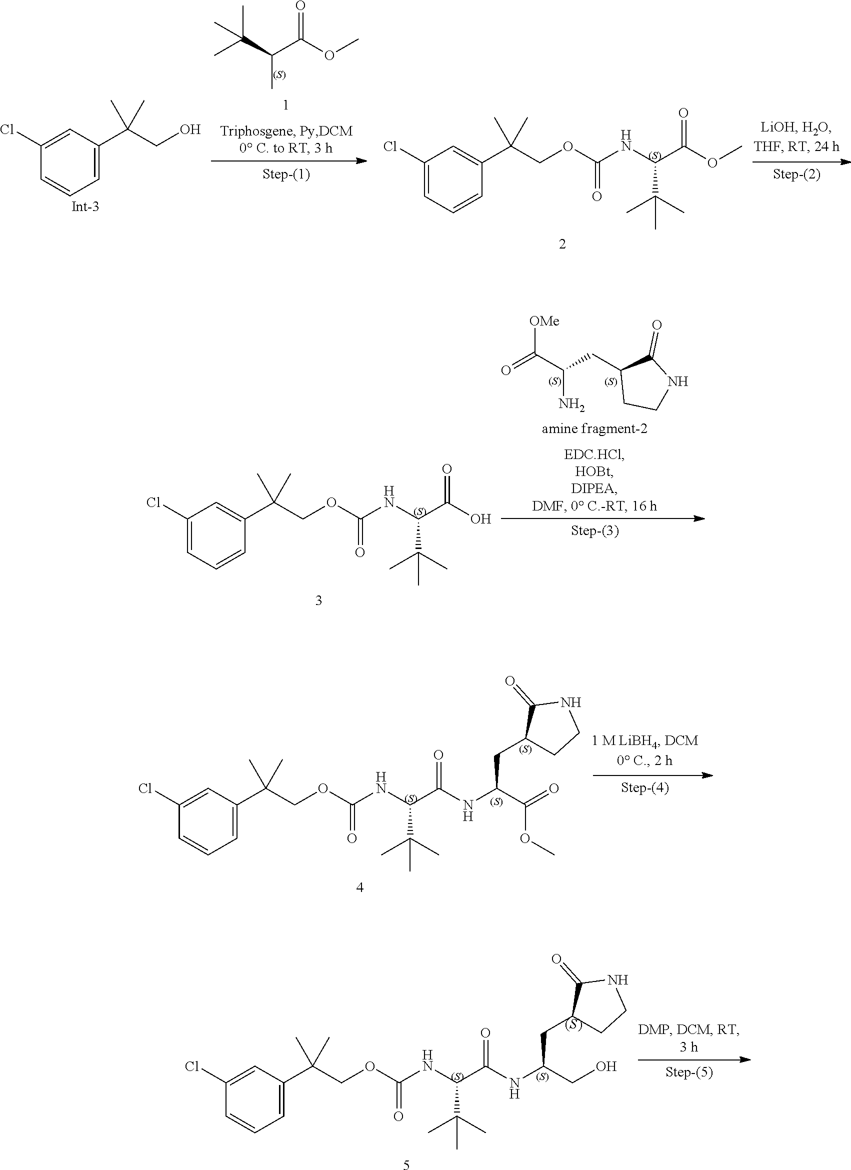

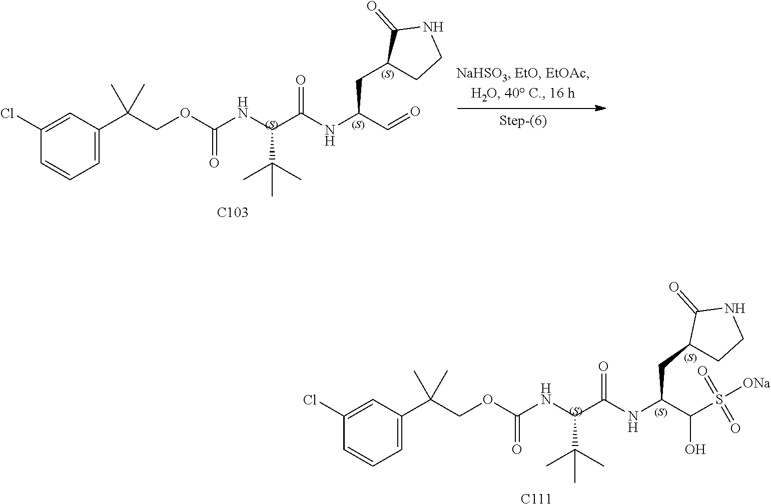

### Methyl (S)-2-(((2-(3-chlorophenyl)-2-methylpropoxy) carbonyl) amino)-3,3-dimethylbutanoate (2)

To a stirred solution of 2-(3-chlorophenyl)-2-methylpropan-1-ol (Int-3) (3.0 g, 16 mmol), methyl (S)-2-amino-3,3-dimethyl butanoate (2.8 g, 19.0 mmol) in DCM (40 mL) was added pyridine (9 mL, 3 vol) followed by triphosgene (2.36 g, 8.0 mmol) at 0° C. and stirred at room temperature for 3 h. The progress of the reaction was monitored by TLC and LCMS. Reaction mixture was quenched with 2N HCl (30 mL), extracted with DCM (2×30 mL). The combined organic layer was dried over sodium sulfate, filtered and evaporated under reduced pressure. The crude residue was purified by combi-flash, compound eluted at 10% ethyl acetate in pet ether to afford methyl (S)-2-(((2-(3-chlorophenyl)-2-methylpropoxy) carbonyl) amino)-3,3-dimethylbutanoatenate (2). TLC system: 20% Ethyl acetate in hexane Rf: 0.5 LCMS (ESI): m/z 356.43 [M+H]⁺

### (S)-2-(((2-(3-Chlorophenyl)-2-methylpropoxy) carbonyl) amino)-3,3-dimethyl butanoic acid (3)

To a stirred solution of methyl (S)-2-(((2-(3-chlorophenyl)-2-methylpropoxy) carbonyl) amino)-3,3-dimethylbutanoatenate (2) (4.1 g, 11.54 mmol) in THF (20 mL), water (20 mL) was added lithium hydroxide (2.7 g, 115.54 mmol) at RT and stirred at room temperature for 24 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford (S)-2-(((2-(3-chlorophenyl)-2-methylpropoxy) carbonyl) amino)-3,3-dimethylbutanoic acid (3). TLC system: 100% EtOAc Rf: 0.1 LCMS (ESI): m/z 342.41 [M+H]⁺

### Methyl (S)-2-((S)-2-(((2-(3-chlorophenyl)-2-methylpropoxy) carbonyl) amino)-3,3-dimethylbutanamido)-3-((S)-2-oxopyrrolidin-3-yl) propanoate (4)

To a stirred solution of (S)-2-(((2-(3-chlorophenyl)-2-methylpropoxy)carbonyl)amino)-3,3-dimethylbutanoic acid (3) (2 g, 5.86 mmol) DMF (15 mL) added EDC.HCl (1.6 g, 8.79 mmol), HOBt (1.1 g, 8.79 mmol), DIPEA (2.5 mL, 17.52 mmol) and methyl (S)-2-amino-3-((S)-2-oxopyrrolidin-3-yl)propanoate hydrochloride (amine fragment-2) (1.3 g, 7.03 mmol) at 0° C. simultaneously and stirred at room temperature for 16 h. Reaction mixture was diluted with ice water (50 mL), extracted with ethyl acetate (2×50 mL), dried over sodium sulfate and evaporated under reduced pressure. The crude residue was purified by combi-flash NP, compound eluted at 4% methanol in dichloromethane to afford methyl (S)-2-((S)-2-(((2-(3-chlorophenyl)-2-methylpropoxy) carbonyl) amino)-3,3-dimethylbutanamido)-3-((S)-2-oxopyrrolidin-3-yl) propanoate (4). TLC system: 10% Methanol in dichloromethane Rf: 0.3 LCMS (ESI): m/z 510.57 [M+H]⁺

### 2-(3-Chlorophenyl)-2-methylpropyl ((S)-1-(((S)-1-hydroxy-3-((S)-2-oxopyrrolidin-3-yl) propan-2-yl) amino)-3,3-dimethyl-1-oxobutan-2-yl) carbamate (5)

To a stirred solution of methyl (S)-2-((S)-2-(((2-(3-chlorophenyl)-2-methylpropoxy) carbonyl)amino)-3,3-dimethylbutanamido)-3-((S)-2-oxopyrrolidin-3-yl)propanoate (4) (500 mg, 0.98 mmol) in DCM (10 mL) was added 2M LiBH₄ in THF (1 mL, 1.96 mmol) at 0° C. and the reaction mixture stirred for 2 h at 0° C. The progress of the reaction was monitored by TLC and LCMS. Reaction mixture was quenched with sat. Ammonium chloride solution (20 mL) and extracted with DCM (2×15 mL). Organic layer was washed with brine solution (30 mL), dried over Na₂SO₄ and concentrated to afford 2-(3-chlorophenyl)-2-methylpropyl ((S)-1-(((S)-1-hydroxy-3-((S)-2-oxopyrrolidin-3-yl)propan-2-yl)amino)-3,3-dimethyl-1-oxobutan-2-yl) carbamate (5). TLC system: 10% Methanol in dichloromethane Rf: 0.2 LCMS (ESI): m/z 482.58 [M+H]⁺

### 2-(3-Chlorophenyl)-2-methylpropyl ((S)-3,3-dimethyl-1-oxo-1-(((S)-1-oxo-3-((S)-2-oxopyrrolidin-3-yl)propan-2-yl)amino)butan-2-yl)carbamate (Compound C103)

To a stirred solution of 2-(3-chlorophenyl)-2-methylpropyl ((S)-1-(((S)-1-hydroxy-3-((S)-2-oxopyrrolidin-3-yl)propan-2-yl)amino)-3,3-dimethyl-1-oxobutan-2-yl)carbamate (5) (200 mg, 0.41 mmol) was dissolved in dichloromethane (5 mL) was added Dess-Martin periodinane (528 mg, 1.24 mmol) at 0° C. and stirred at RT for 3 h. Reaction mixture was diluted with DCM (15 mL) followed by sat. Hypo solution (3×15 mL), followed by sat. NaHCO₃ solution (3×15 mL). Organic layer was dried over anhydrous Na₂SO₄, filtered and concentrated to get crude compound. The crude compound was purified by prep HPLC to afford 2-(3-chlorophenyl)-2-methylpropyl ((S)-3,3-dimethyl-1-oxo-1-(((S)-1-oxo-3-((S)-2-oxopyrrolidin-3-yl) propan-2-yl)amino) butan-2-yl) carbamate (Compound C103). TLC system: 10% Methanol in dichloromethane Rf: 0.45 LCMS (ESI): m/z 480.20 (M+H)⁺

### Sodium (2S)-2-((S)-2-(((2-(3-chlorophenyl)-2-methylpropoxy)carbonyl)amino)-3,3-dimethylbutanamido)-1-hydroxy-3-((S)-2-oxopyrrolidin-3-yl)propane-1-sulfonate (Compound C111)

To a stirred solution of 2-(3-chlorophenyl)-2-methylpropyl ((S)-3,3-dimethyl-1-oxo-1-(((S)-1-oxo-3-((S)-2-oxopyrrolidin-3-yl)propan-2-yl)amino)butan-2-yl)carbamate (Compound C103) (100 mg, 0.20 mmol) in ethanol (2 mL), EtOAc (1 mL), water (1 mL) was added NaHSO₃ (43 mg, 0.41 mmol) at RT and heated to 50° C. for 16 h. The progress of the reaction was monitored by TLC and LCMS. After 16 h, the reaction mixture was cooled to RT and filtered through celite pad then washed with ethanol (5 mL). Filtrate was evaporated under reduce pressure to afford crude residue. This residue was triturated with di ethyl ether, EtOAc for 3 times to afford sodium (2S)-2-((S)-2-(((2-(3-chlorophenyl)-2-methylpropoxy) carbonyl)amino)-3,3-dimethylbutanamido)-1-hydroxy-3-((S)-2-oxopyrrolidin-3-yl)propane-1-sulfonate (Compound C111). TLC system: 15% Methanol in dichloromethane Rf: 0.1 LCMS (ESI): m/z 560.1 [M−Na]⁻

### Example 81: Synthesis of Compounds C104 and C114

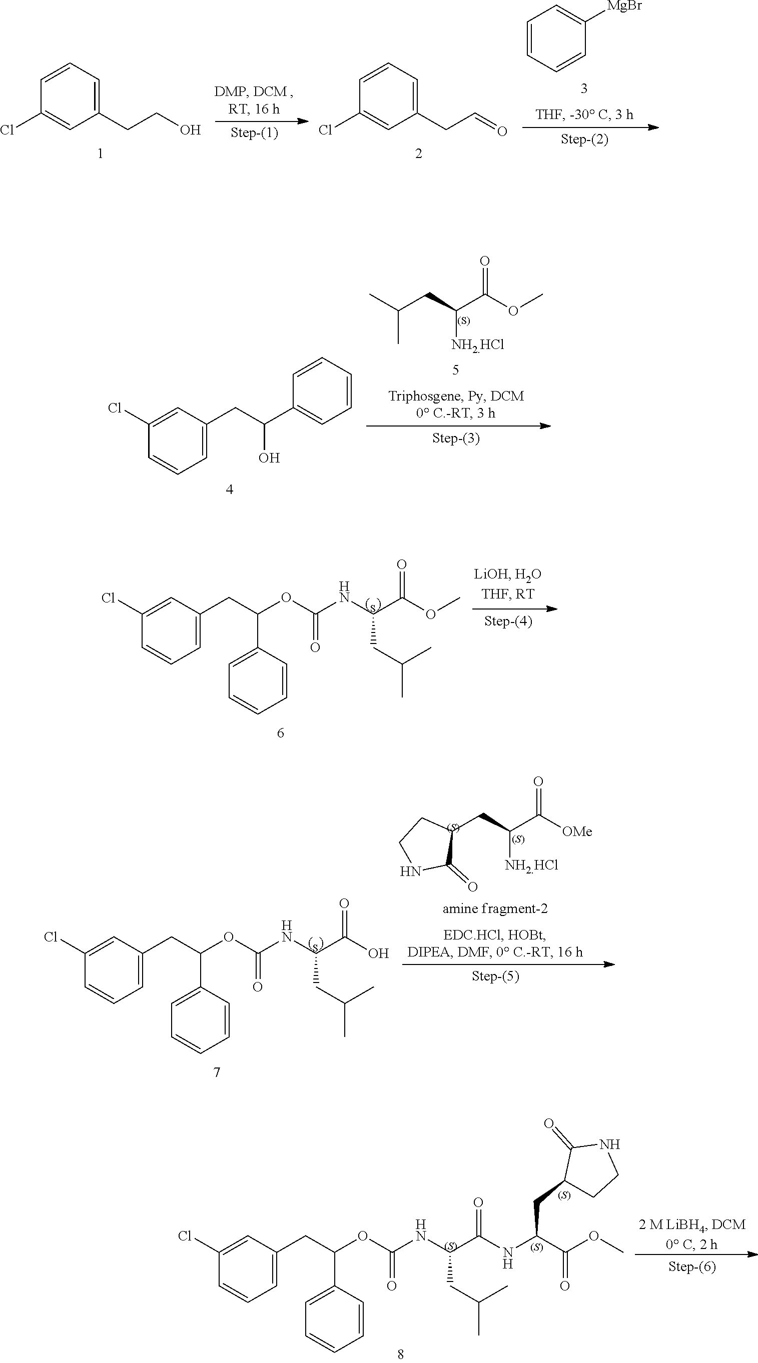

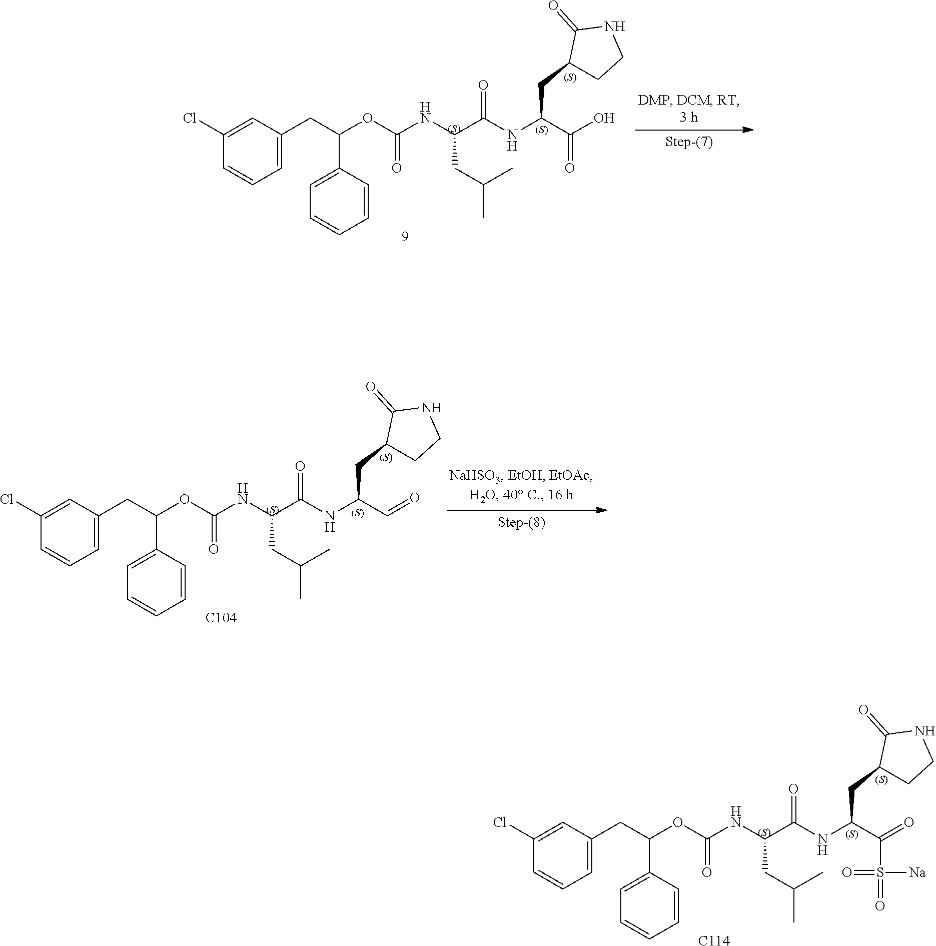

### 2-(3-Chlorophenyl) acetaldehyde (2)

To a stirred solution of 2-(3-chlorophenyl)ethan-1-ol (1) (20 g, 128.20 mmol) in DCM (200 mL) was added Dess-Martin periodinane (16.3 g, 384.61 mmol) at 0° C. and stirred at RT for 16 h. The progress of the reaction was monitored by TLC. Reaction mixture was filtered through the celite bed and wash with DCM (80 mL). Filtrate was washed with sat. Hypo solution (3×150 mL), sat. NaHCO₃ solution (3×150 mL) followed by brine (100 mL), dried over sodium sulfate and concentrated under reduced pressure to afford crude; The crude was purified by normal phase chromatography to afford 2-(3-chlorophenyl) acetaldehyde (2). TLC system: 10% Ethyl acetate in hexane Rf: 0.5 LCMS (ESI): m/z 156.18 [M+1]⁺

### 2-(3-chlorophenyl)-1-phenylethan-1-ol (4)

To a stirred solution of phenylmagnesium bromide (3) (155 mL, 155.84 mmol) in tetrahydrofuran (120 mL) was added 2-(3-chlorophenyl)acetaldehyde (12 g, 77.92 mmol) at −30° C. and stirred at RT for 3 h. The progress of the reaction was monitored by TLC. Reaction mixture was quenched with saturated ammonium chloride solution and extracted with ethyl acetate (2×100 mL). Combined organic layer and washed with water (2×50 mL), dried over sodium sulfate, concentrated under reduced pressure to afford crude residue, crude residue was purified by normal phase chromatography to afford 2-(3-chlorophenyl)-1-phenylethan-1-ol (4). TLC system: 10% Ethyl acetate in hexane Rf: 0.7 LCMS (ESI): m/z 215.18 [M−OH]⁺

### Methyl ((2-(3-chlorophenyl)-1-phenylethoxy) carbonyl)-L-leucinate (6)

To a stirred solution of 2-(3-chlorophenyl)-1-phenylethan-1-ol (4) (4 g, 17.23 mmol), methyl L-leucinate hydrochloride (5) (3.7 g, 25.85 mmol) in DCM (40 mL) was added pyridine (12 mL, 3 vol) followed by triphosgene (2.5 g, 8.61 mmol) at 0° C. and stirred at room temperature for 3 h. The progress of the reaction was monitored by TLC and LCMS. Reaction mixture was diluted with DCM and washed with 1N HCl (50 mL), organic layer was dried over sodium sulfate, filtered and evaporated under reduced pressure. The crude residue was purified by combi-flash, compound eluted at 10% ethyl acetate in pet ether to afford methyl ((2-(3-chlorophenyl)-1-phenylethoxy) carbonyl)-L-leucinate (6). TLC system: 10% Ethyl acetate in hexane Rf: 0.3 LCMS (ESI): m/z 426.43 [M+Na]⁺

### ((2-(3-Chlorophenyl)-1-phenylethoxy) carbonyl)-L-leucine (7)

To a stirred solution of methyl ((2-(3-chlorophenyl)-1-phenylethoxy)carbonyl)-L-leucinate (6) (2.2 g, 5.45 mmol) in THF (30 mL), water (15 mL) was added lithium hydroxide (671 mg, 16.37 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford ((2-(3-chlorophenyl)-1-phenylethoxy)carbonyl)-L-leucine (7). TLC system: 100% EtOAc Rf: 0.1 LCMS (ESI): m/z 412.41 [M+Na]⁺

### Methyl (2S)-2-((2S)-2-(((2-(3-chlorophenyl)-1-phenylethoxy) carbonyl) amino)-4-methylpentanamido)-3-((S)-2-oxopyrrolidin-3-yl) propanoate (8)

To a stirred solution of ((2-(3-chlorophenyl)-1-phenylethoxy)carbonyl)-L-leucine (7) (1.7 g, 4.36 mmol) DMF (20 mL) added EDC.HCl (1.2 g, 6.55 mmol), HOBt (0.88 g, 6.55 mmol), DIPEA (1.8 mL, 13.10 mmol) and methyl (S)-2-amino-3-((S)-2-oxopyrrolidin-3-yl)propanoate hydrochloride (amine fragment-2) (0.97 g, 5.24 mmol) at 0° C. simultaneously and stirred at room temperature for 16 h. Reaction mixture was quenched with ice water (80 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% methanol in dichloromethane to afford methyl (2S)-2-((2S)-2-(((2-(3-chlorophenyl)-1-phenylethoxy) carbonyl) amino)-4-methyl pentanamido)-3-((S)-2-oxopyrrolidin-3-yl) propanoate (8). TLC system: 10% Methanol in dichloromethane Rf: 0.4 LCMS (ESI): m/z 558.57 [M+H]⁺

### 2-(3-Chlorophenyl)-1-phenylethyl ((S)-1-(((S)-1-hydroxy-3-((S)-2-oxopyrrolidin-3-yl) propan-2-yl) amino)-4-methyl-1-oxopentan-2-yl) carbamate (9)

To a stirred solution of methyl (2S)-2-((2S)-2-(((2-(3-chlorophenyl)-1-phenylethoxy)carbonyl)amino)-4-methylpentanamido)-3-((S)-2-oxopyrrolidin-3-yl)propanoate (8) (800 mg, 1.43 mmol) in DCM (10 mL) was added 2M LiBH₄ in THF (1.4 mL, 2.87 mmol) at 0° C. and the reaction mixture stirred for 2 h at 0° C. The progress of the reaction was monitored by TLC and LCMS. Reaction mixture was quenched with sat. Ammonium chloride solution (20 mL) and extracted with DCM (2×20 mL). Organic layer was washed with brine solution (30 mL), dried over Na₂SO₄ and concentrated to afford 2-(3-chlorophenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (9). TLC system: 10% Methanol in dichloromethane Rf: 0.2 LCMS (ESI): m/z 530.60 [M+H]⁺

### 2-(3-Chlorophenyl)-1-phenylethyl ((S)-4-methyl-1-oxo-1-(((S)-1-oxo-3-((S)-2-oxopyrrolidin-3-yl) propan-2-yl) amino) pentan-2-yl) carbamate (Compound C104)

To a stirred solution of 2-(3-chlorophenyl)-1-phenylethyl ((S)-1-(((S)-1-hydroxy-3-((S)-2-oxopyrrolidin-3-yl)propan-2-yl)amino)-4-methyl-1-oxopentan-2-yl)carbamate (9) (250 mg, 0.47 mmol) was dissolved in dichloromethane (5 mL) was added Dess-Martin periodinane (600 mg, 1.41 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford 2-(3-chlorophenyl)-1-phenylethyl ((S)-4-methyl-1-oxo-1-(((S)-1-oxo-3-((S)-2-oxopyrrolidin-3-yl)propan-2-yl) amino) pentan-2-yl)carbamate (Compound C104). TLC system: 10% Methanol in dichloromethane Rf: 0.5 LCMS (ESI): m/z 528.2 (M+H)⁺

### Sodium (2S)-2-((2S)-2-(((2-(3-chlorophenyl)-1-phenylethoxy) carbonyl) amino)-4-methylpentanamido)-1-hydroxy-3-((S)-2-oxopyrrolidin-3-yl) propane-1-sulfonate (Compound C114)

To a stirred solution of 2-(3-chlorophenyl)-2-methylpropyl ((S)-3,3-dimethyl-1-oxo-1-(((S)-1-oxo-3-((S)-2-oxopyrrolidin-3-yl)propan-2-yl)amino)butan-2-yl)carbamate (Compound C104) (100 mg, 0.18 mmol) in ethanol (0.5 mL), EtOAc (1 mL), water (0.5 mL) was added NaHSO₃ (39 mg, 0.37 mmol) at RT and heated to 50° C. for 16 h. The progress of the reaction was monitored by TLC and LCMS. After 16 h, the reaction mixture was cooled to RT and filtered through celite pad then washed with ethanol (5 mL). Filtrate was evaporated under reduce pressure to afford crude residue. This residue was triturated with di ethyl ether, EtOAc for 3 times to afford sodium (2S)-2-((2S)-2-(((2-(3-chlorophenyl)-1-phenylethoxy)carbonyl) amino)-4-methylpentanamido)-1-hydroxy-3-((S)-2-oxopyrrolidin-3-yl)propane-1-sulfonate (Compound C114). TLC system: 15% Methanol in dichloromethane Rf: 0.1 LCMS (ESI): m/z 608.2 [M−Na]⁻

### Example 82: Synthesis of Compound C107

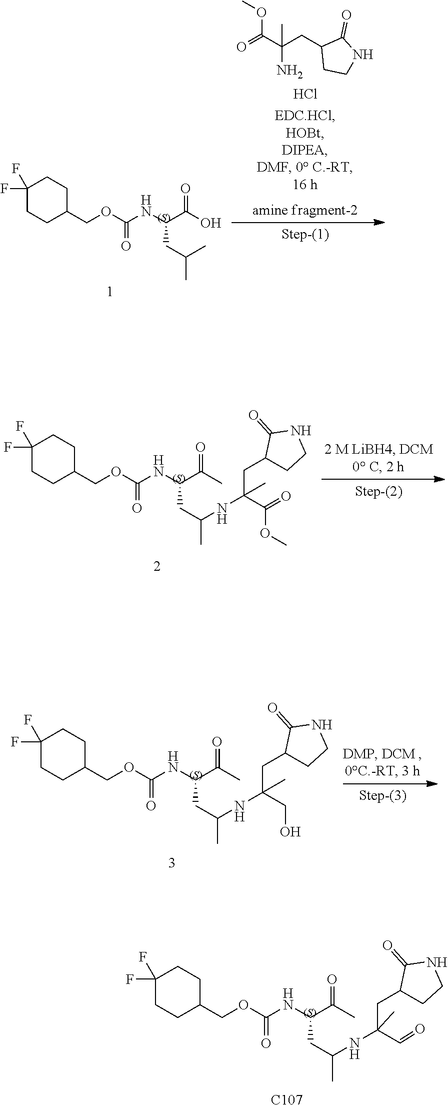

### Methyl 2-((S)-2-((((4,4-difluorocyclohexyl) methoxy) carbonyl) amino)-4-methylpentanamido)-2-methyl-3-(2-oxopyrrolidin-3-yl) propanoate (2)

To a stirred solution of (((4,4-difluorocyclohexyl)methoxy)carbonyl)-L-leucine (1) (600 mg, 1.95 mmol) DMF (10 mL) added EDC.HCl (559 mg, 2.92 mmol), HOBt (395 mg, 2.92 mmol), DIPEA (1 mL, 5.86 mmol) and methyl 2-amino-2-methyl-3-(2-oxopyrrolidin-3-yl)propanoate hydrochloride (amine fragment-2A) (469 mg, 2.29 mmol) at 0° C. simultaneously and stirred at room temperature for 16 h. Reaction mixture was diluted with ice water (30 mL), extracted with ethyl acetate (2×20 mL), dried over sodium sulfate and evaporated under reduced pressure. The crude residue was purified by combi-flash NP, compound eluted at 5% methanol in dichloromethane to afford methyl 2-((S)-2-((((4,4-difluorocyclohexyl) methoxy) carbonyl) amino)-4-methylpentanamido)-2-methyl-3-(2-oxopyrrolidin-3-yl) propanoate (2). TLC system: 5% MeOH in DCM Rf: 0.4 LCMS (ESI): m/z 490.40 [M+H]⁺

### (4,4-Difluorocyclohexyl)methyl ((2S)-1-((1-hydroxy-2-methyl-3-(2-oxopyrrolidin-3-yl)propan-2-yl)amino)-4-methyl-1-oxopentan-2-yl)carbamate (3)

To a stirred solution of methyl 2-((S)-2-((((4,4-difluorocyclohexyl) methoxy) carbonyl) amino)-4-methylpentanamido)-2-methyl-3-(2-oxopyrrolidin-3-yl) propanoate (2) (750 mg, 1.53 mmol) in DCM (10 mL) was added 2M LiBH₄ in THF (1.5 mL, 3.06 mmol) at 0° C. and the reaction mixture stirred for 2 h at 0° C. The progress of the reaction was monitored by TLC and LCMS. Reaction mixture was quenched with sat. ammonium chloride solution (30 mL) and extracted with DCM (2×15 mL). Organic layer was washed with brine solution (20 mL), dried over Na₂SO₄ and concentrated to afford (4,4-difluorocyclohexyl) methyl ((2S)-1-((1-hydroxy-2-methyl-3-(2-oxopyrrolidin-3-yl) propan-2-yl) amino)-4-methyl-1-oxopentan-2-yl) carbamate (3). TLC system: 10% MeOH in DCM Rf: 0.3 LCMS (ESI): m/z 462.49 [M+H]⁺

### (4,4-Difluorocyclohexyl) methyl ((2S)-4-methyl-1-((2-methyl-1-oxo-3-(2-oxopyrrolidin-3-yl) propan-2-yl) amino)-1-oxopentan-2-yl) carbamate (Compound C107)

To a stirred solution of (4,4-difluorocyclohexyl)methyl ((2S)-1-((1-hydroxy-2-methyl-3-(2-oxopyrrolidin-3-yl)propan-2-yl)amino)-4-methyl-1-oxopentan-2-yl)carbamate (3) (500 mg, 1.08 mmol) was dissolved in dichloromethane (15 mL) was added Dess-Martin periodinane (1.3 g, 3.25 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford (4,4-difluorocyclohexyl) methyl ((2S)-4-methyl-1-((2-methyl-1-oxo-3-(2-oxopyrrolidin-3-yl) propan-2-yl) amino)-1-oxopentan-2-yl) carbamate (Compound C107). TLC system: 10% MeOH in DCM Rf: 0.6 LCMS (ESI): m/z 460.2 (M+H)⁺

### Example 83: Synthesis of Compound C108

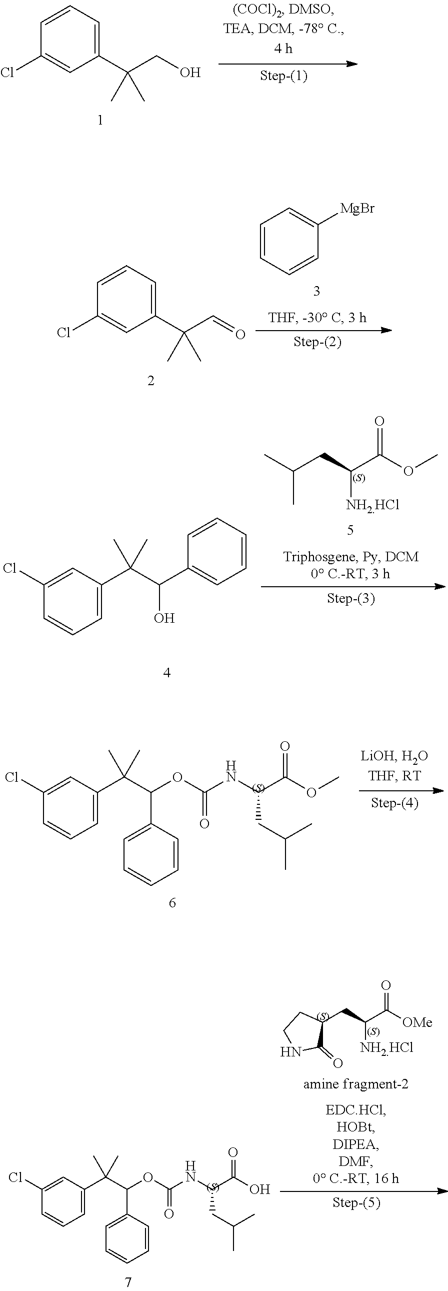

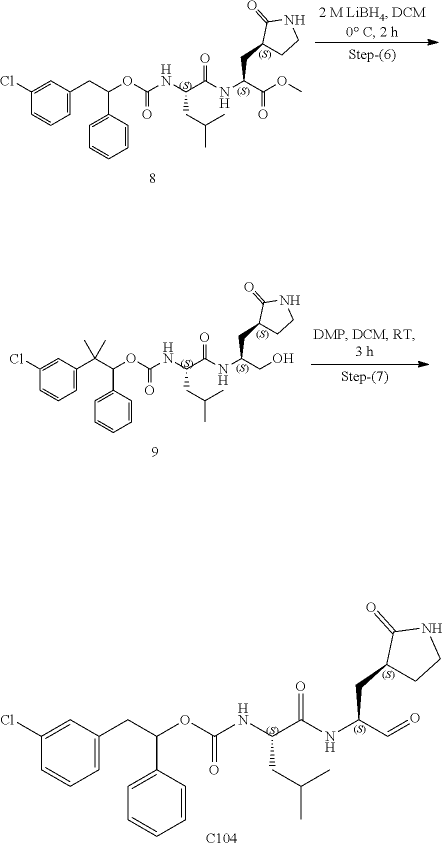

### 2-(3-Chlorophenyl)-2-methylpropanal (2)

To a stirred solution of DMSO (7.6 mL, 108.69 mmol) in DCM (100 mL) was added oxalyl chloride (7 mL, 81.52 mmol) at −78° C. and stirred for 30 min then added 2-(3-chlorophenyl)-2-methylpropan-1-ol (10 g, 54.34 mmol) in DCM at −78° C. and continued for 2 h after that TEA (45.6 mL, 326.08 mmol) was added at same temperature and stirred at RT for 3 h. The progress of the reaction was monitored by TLC and LCMS. Reaction mixture was diluted with DCM (100 mL) and washed with ice cold water (3×100 mL), dried over sodium sulfate, concentrated under reduced pressure to afford crude 2-(3-chlorophenyl)-2-methylpropanal (2) which was used directly in the next step. TLC system: 20% Ethyl acetate in hexane Rf: 0.6 LCMS (ESI): m/z 148.07 [M+H]⁺

### 2-(3-Chlorophenyl)-2-methyl-1-phenylpropan-1-ol (4)

To a stirred solution of phenyl magnesium bromide (3) (85 mL, 82.87 mmol) in tetrahydrofuran (100 mL) was added 2-(3-chlorophenyl)-2-methylpropanal (10 g, 55.24 mmol) at −30° C. and stirred at RT for 3 h. The progress of the reaction was monitored by TLC and LCMS. Reaction mixture was quenched with saturated ammonium chloride solution and extracted with ethyl acetate (2×50 mL). Combined organic layer and washed with water (2×50 mL), dried over sodium sulfate, concentrated under reduced pressure to afford crude residue, crude residue was purified by normal phase chromatography to afford 2-(3-chlorophenyl)-2-methyl-1-phenylpropan-1-ol (4). TLC system: 20% Ethyl acetate in hexane Rf: 0.3 LCMS (ESI): m/z 243.01 [M−OH]⁺

### Methyl ((2-(3-chlorophenyl)-2-methyl-1-phenylpropoxy) carbonyl)-leucinate (4)

To a stirred solution of 2-(3-chlorophenyl)-2-methyl-1-phenylpropan-1-ol (4) (3.6 g, 13.84 mmol), methyl (S)-2-amino-3,3-dimethylbutanoate HCl (2.4 g, 16.61 mmol) in DCM (40 mL) was added pyridine (10.8 mL, 3 vol) followed by triphosgene (2.0 g, 6.75 mmol) at 0° C. and stirred at room temperature for 3 h. The progress of the reaction was monitored by TLC and LCMS. Reaction mixture was quenched with 2N HCl (50 mL), extracted with DCM (2×40 mL). The combined organic layer was dried over sodium sulfate, filtered and evaporated under reduced pressure. The crude residue was purified by combi-flash, compound eluted at 10% ethyl acetate in pet ether to afford methyl ((2-(3-chlorophenyl)-2-methyl-1-phenylpropoxy) carbonyl)-leucinate (6). TLC system: 30% Ethyl acetate in hexane Rf: 0.3 LCMS (ESI): m/z 454.48 [M+Na]⁺

### ((2-(3-Chlorophenyl)-2-methyl-1-phenylpropoxy) carbonyl)-L-leucine (7)

To a stirred solution of methyl ((2-(3-chlorophenyl)-2-methyl-1-phenylpropoxy) carbonyl)-L-leucinate (6) (3.0 g, 6.60 mmol) in THF (20 mL), water (20 mL) was added lithium hydroxide (0.81 g, 19.82 mmol) at 0° C. and stirred at room temperature for 2 h. The progress of the reaction was monitored by TLC and LCMS. Reaction mixture completely distilled under reduced pressure, crude compound acidified with aq. 1N HCl solution up to pH˜3 and extracted with ethyl acetate (2×40 mL), dried over sodium sulfate, concentrated under reduced pressure to afford ((2-(3-chlorophenyl)-2-methyl-1-phenylpropoxy)carbonyl)-L-leucine (7). TLC system: 100% EtOAc Rf: 0.1 LCMS (ESI): m/z 440.47 [M+Na]⁺

### Methyl (2S)-2-((2S)-2-(((2-(3-chlorophenyl)-2-methyl-1-phenylpropoxy) carbonyl) amino)-4-methylpentanamido)-3-((S)-2-oxopyrrolidin-3-yl) propanoate (8)

To a stirred solution of ((2-(3-chlorophenyl)-2-methyl-1-phenylpropoxy)carbonyl)-L-leucine (7) (2.2 g, 5.27 mmol) DMF (15 mL) added EDC.HCl (1.5 g, 7.85 mmol), HOBt (1.0 g, 7.40 mmol), DIPEA (2.8 mL, 16.18 mmol) and methyl (S)-2-amino-3-((S)-2-oxopyrrolidin-3-yl)propanoate hydrochloride (amine fragment-2) (1.2 g, 6.33 mmol) at 0° C. simultaneously and stirred at room temperature for 16 h. Reaction mixture was diluted with ice water (80 mL), extracted with ethyl acetate (2×50 mL), dried over sodium sulfate and evaporated under reduced pressure. The crude residue was purified by combi-flash NP, compound eluted at 3% methanol in dichloromethane to afford methyl (2S)-2-((2S)-2-(((2-(3-chlorophenyl)-2-methyl-1-phenylpropoxy)carbonyl)amino)-4-methylpentanamido)-3-((S)-2-oxopyrrolidin-3-yl)propanoate (8). TLC system: 10% Methanol in dichloromethane Rf: 0.4 LCMS (ESI): m/z 586.62 [M+H]⁺

### 2-(3-Chlorophenyl)-2-methyl-1-phenylpropyl ((S)-1-(((S)-1-hydroxy-3-((S)-2-oxopyrrolidin-3-yl) propan-2-yl) amino)-4-methyl-1-oxopentan-2-yl) carbamate (9)

To a stirred solution of methyl (2S)-2-((2S)-2-(((2-(3-chlorophenyl)-2-methyl-1-phenylpropoxy)carbonyl)amino)-4-methylpentanamido)-3-((S)-2-oxopyrrolidin-3-yl)propanoate (8) (1.5 g, 2.56 mmol) in DCM (20 mL) was added 2M LiBH₄ in THF (2.5 mL, 5.12 mmol) at 0° C. and the reaction mixture stirred for 2 h at 0° C. The progress of the reaction was monitored by TLC and LCMS. Reaction mixture was quenched with sat. Ammonium chloride solution (30 mL) and extracted with DCM (2×30 mL). Organic layer was washed with brine solution (30 mL), dried over Na₂SO₄ and concentrated to afford 2-(3-chlorophenyl)-2-methyl-1-phenylpropyl ((S)-1-(((S)-1-hydroxy-3-((S)-2-oxopyrrolidin-3-yl)propan-2-yl)amino)-4-methyl-1-oxopentan-2-yl)carbamate (9). TLC system: 10% Methanol in dichloromethane Rf: 0.2 LCMS (ESI): m/z 580.96 [M+Na]⁺

### 2-(3-Chlorophenyl)-2-methyl-1-phenylpropyl ((S)-4-methyl-1-oxo-1-(((S)-1-oxo-3-((S)-2-oxopyrrolidin-3-yl) propan-2-yl) amino) pentan-2-yl) carbamate (Compound C108)

To a stirred solution of 2-(3-chlorophenyl)-2-methyl-1-phenylpropyl ((S)-1-(((S)-1-hydroxy-3-((S)-2-oxopyrrolidin-3-yl)propan-2-yl)amino)-4-methyl-1-oxopentan-2-yl)carbamate (9) (250 mg, 0.44 mmol) was dissolved in dichloromethane (10 mL) was added Dess-Martin periodinane (570 mg, 1.34 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford 2-(3-chlorophenyl)-2-methyl-1-phenylpropyl ((S)-4-methyl-1-oxo-1-(((S)-1-oxo-3-((S)-2-oxopyrrolidin-3-yl)propan-2-yl)amino)pentan-2-yl)carbamate (Compound C108). TLC system: 10% Methanol in dichloromethane Rf: 0.5 LCMS (ESI): m/z 545.45 (M+H)⁺

### Example 84: Synthesis of Compounds C109 and C115

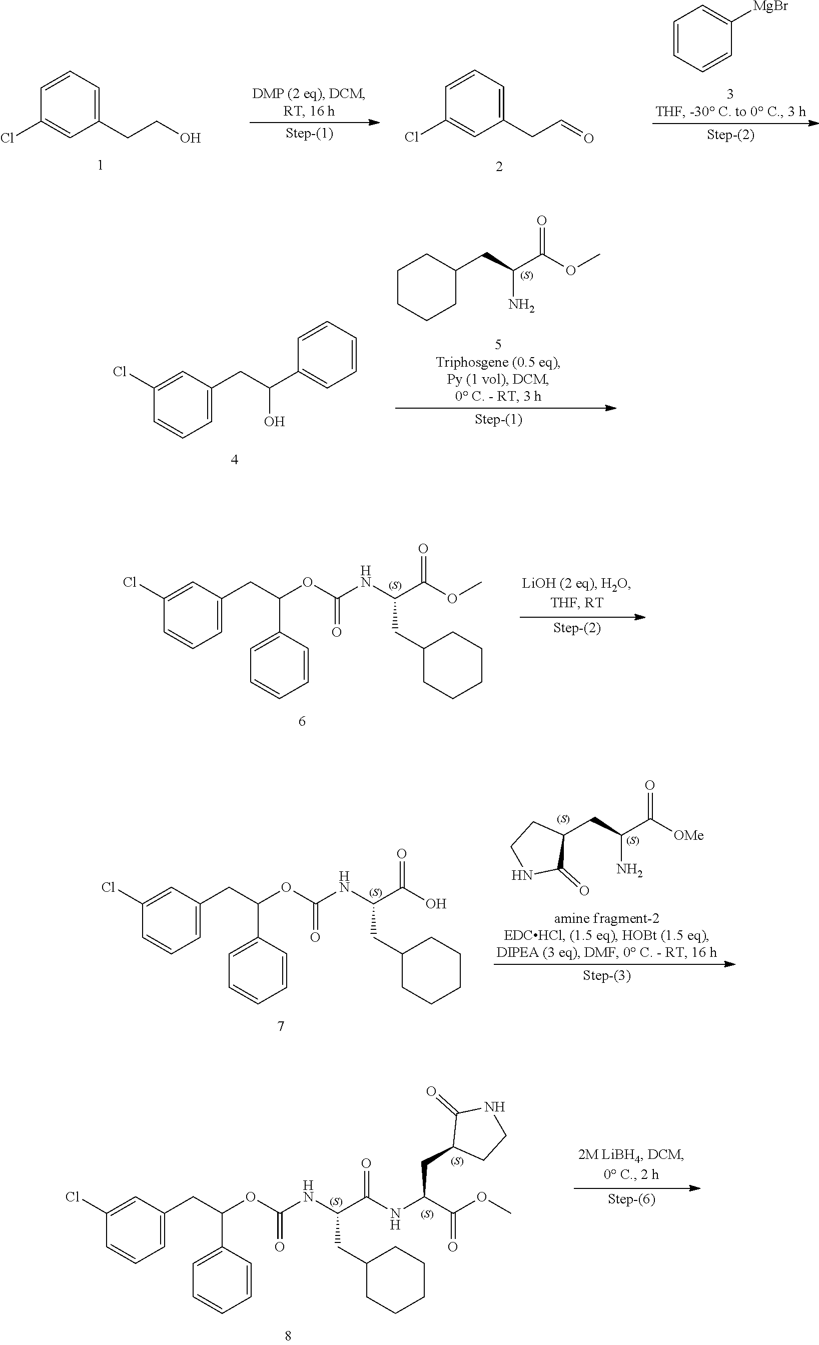

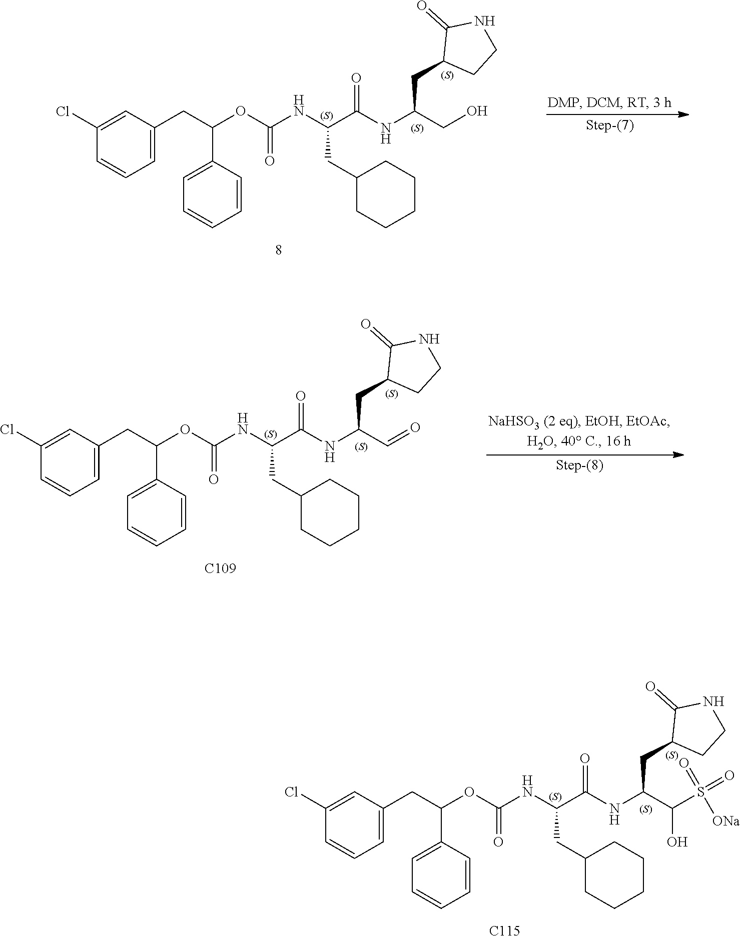

### 2-(3-Chlorophenyl)acetaldehyde (2)

To a stirred solution of 2-(3-chlorophenyl)ethan-1-ol (1) (20 g, 128.20 mmol) in DCM (200 mL) was added Dess-Martin periodinane (16.3 g, 38.46 mmol) at 0° C. and stirred at RT for 3 h. The progress of the reaction was monitored by TLC. Reaction mixture was filtered through the celite bed and wash with DCM (80 mL) followed by sat. Hypo solution (3×160 mL), followed by sat. NaHCO₃ solution (3×120 mL) the organic layer and washed with brine (100 mL), Organic layer was dried over sodium sulphate, concentrated to get crude compound. The crude compound was purified by normal phase chromatography to afford 2-(3-chlorophenyl)acetaldehyde (2). TLC system: 10% Ethyl acetate in hexane Rf: 0.5 LCMS (ESI): m/z 155.15 [M+H]⁺

### 2-(3-Chlorophenyl)-1-phenylethan-1-ol (4)

To a stirred solution of phenylmagnesium bromide (3) (155 mL, 155.84 mmol) in tetrahydrofuran (120 mL) was added 2-(3-chlorophenyl)acetaldehyde (12 g, 77.92 mmol) at −30° C. and stirred at RT for 3 h. The progress of the reaction was monitored by TLC. Reaction mixture was quenched with saturated ammonium chloride solution and extracted with ethyl acetate (2×100 mL). Combined organic layer and washed with water (2×50 mL), dried over sodium sulfate, concentrated under reduced pressure to afford crude residue, crude residue was purified by normal phase chromatography to afford 2-(3-chlorophenyl)-1-phenylethan-1-ol (4). TLC system: 10% Ethyl acetate in hexane Rf: 0.7 LCMS (ESI): m/z 215.18 [M−OH]⁻

### Methyl (2S)-2-(((2-(3-chlorophenyl)-1-phenylethoxy)carbonyl)amino)-3-cyclohexylpropanoate (6)

To a stirred solution of 2-(3-chlorophenyl)-1-phenylethan-1-ol (4) (3.5 g, 15.08 mmol), methyl (S)-2-amino-3-cyclohexylpropanoate (5) (3.6 g, 19.60 mmol) in DCM (35 mL) was added pyridine (10.5 mL, 3 vol) followed by triphosgene (2.2 g, 7.54 mmol) at 0° C. and stirred at room temperature for 3 h. The progress of the reaction was monitored by TLC and LCMS. Reaction mixture was quenched with 1N HCl (50 mL), extracted with DCM (2×60 mL). The combined organic layer was dried over sodium sulfate, filtered and evaporated under reduced pressure. The crude residue was purified by combi-flash, compound eluted at 10% ethyl acetate in pet ether methyl (2S)-2-(((2-(3-chlorophenyl)-1-phenylethoxy)carbonyl)amino)-3-cyclohexylpropanoate (6). TLC system: 10% Ethyl acetate in hexane Rf: 0.3 LCMS (ESI): m/z 466.49 [M+Na]⁺

### (2S)-2-(((2-(3-Chlorophenyl)-1-phenylethoxy)carbonyl)amino)-3-cyclohexylpropanoic acid (7)

To a stirred solution of methyl (2S)-2-(((2-(3-chlorophenyl)-1-phenylethoxy)carbonyl)amino)-3-cyclohexylpropanoate (6) (3.3 g, 7.44 mmol) in THF (40 mL), water (20 mL) was added lithium hydroxide (915 mg, 22.33 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford (2S)-2-(((2-(3-chlorophenyl)-1-phenylethoxy)carbonyl)amino)-3-cyclohexylpropanoic acid (7). TLC system: 100% EtOAc Rf: 0.1 LCMS (ESI): m/z 452.44 [M+Na]⁺

### Methyl (2S)-2-((2S)-2-(((2-(3-chlorophenyl)-1-phenylethoxy)carbonyl)amino)-3-cyclohexylpropanamido)-3-((S)-2-oxopyrrolidin-3-yl)propanoate (8)

To a stirred solution of (2S)-2-(((2-(3-chlorophenyl)-1-phenylethoxy)carbonyl)amino)-3-cyclohexylpropanoic acid (7) (3 g, 6.99 mmol) DMF (30 mL) added EDC.HCl (2 g, 10.48 mmol), HOBt (1.4 g, 10.48 mmol), DIPEA (3 mL, 20.97 mmol) and methyl (S)-2-amino-3-((S)-2-oxopyrrolidin-3-yl)propanoate hydrochloride (amine fragment-2) (1.5 g, 8.38 mmol) at 0° C. simultaneously and stirred at room temperature for 16 h. Reaction mixture was diluted with ice water (80 mL), extracted with ethyl acetate (2×60 mL), dried over sodium sulfate and evaporated under reduced pressure. The crude residue was purified by combi-flash NP, compound eluted at 3% methanol in dichloromethane to methyl (2S)-2-((2S)-2-(((2-(3-chlorophenyl)-1-phenylethoxy)carbonyl)amino)-3-cyclohexylpropanamido)-3-((S)-2-oxopyrrolidin-3-yl)propanoate (8). TLC system: 10% Methanol in dichloromethane Rf: 0.4 LCMS (ESI): m/z 598.30 [M+H]⁺

### 2-(3-Chlorophenyl)-1-phenylethyl ((S)-3-cyclohexyl-1-(((S)-1-hydroxy-3-((S)-2-oxopyrrolidin-3-yl)propan-2-yl)amino)-1-oxopropan-2-yl)carbamate (9)

To a stirred solution of methyl (2S)-2-((2S)-2-(((2-(3-chlorophenyl)-1-phenylethoxy)carbonyl)amino)-3-cyclohexylpropanamido)-3-((S)-2-oxopyrrolidin-3-yl)propanoate (8) (1 g, 1.67 mmol) in DCM (10 mL) was added 2M LiBH₄ in THF (1.6 mL, 3.34 mmol) at 0° C. and the reaction mixture stirred for 2 h at 0° C. The progress of the reaction was monitored by TLC and LCMS. Reaction mixture was quenched with sat. Ammonium chloride solution (20 mL) and extracted with DCM (2×20 mL). Organic layer was washed with brine solution (30 mL), dried over Na₂SO₄ and concentrated to afford 2-(3-chlorophenyl)-1-phenylethyl ((S)-3-cyclohexyl-1-(((S)-1-hydroxy-3-((S)-2-oxopyrrolidin-3-yl)propan-2-yl)amino)-1-oxopropan-2-yl)carbamate (9). TLC system: 10% Methanol in dichloromethane Rf: 0.2 LCMS (ESI): m/z 570.66 [M+H]⁺

### 2-(3-Chlorophenyl)-1-phenylethyl ((S)-3-cyclohexyl-1-oxo-1-(((S)-1-oxo-3-((S)-2-oxopyrrolidin-3-yl)propan-2-yl)amino)propan-2-yl)carbamate (Compound C109)

To a stirred solution of 2-(3-chlorophenyl)-1-phenylethyl ((S)-3-cyclohexyl-1-(((S)-1-hydroxy-3-((S)-2-oxopyrrolidin-3-yl)propan-2-yl)amino)-1-oxopropan-2-yl)carbamate (9) (700 mg, 1.22 mmol) was dissolved in dichloromethane (12 mL) was added Dess-Martin periodinane (1.5 mg, 3.68 mmol) at 0° C. and stirred at RT for 3 h. Reaction mixture was diluted with DCM (600 mL) followed by sat. Hypo solution (3×30 mL), followed by sat. NaHCO₃ solution (3×30 mL). Organic layer was dried over anhydrous Na₂SO₄, filtered and concentrated to get crude compound. The crude compound was purified by prep HPLC to afford 2-(3-chlorophenyl)-1-phenylethyl ((S)-3-cyclohexyl-1-oxo-1-(((S)-1-oxo-3-((S)-2-oxopyrrolidin-3-yl)propan-2-yl)amino)propan-2-yl)carbamate (Compound C109). TLC system: 10% Methanol in dichloromethane Rf: 0.5 LCMS (ESI): m/z 568.2 (M+H)⁺

### Sodium (2S)-2-((2S)-2-(((2-(3-chlorophenyl)-1-phenylethoxy)carbonyl)amino)-3-cyclohexylpropanamido)-1-hydroxy-3-((S)-2-oxopyrrolidin-3-yl)propane-1-sulfonate (Compound C115)

To a stirred solution of 2-(3-chlorophenyl)-1-phenylethyl ((S)-3-cyclohexyl-1-oxo-1-(((S)-1-oxo-3-((S)-2-oxopyrrolidin-3-yl)propan-2-yl)amino)propan-2-yl)carbamate (Compound C109) (100 mg, 0.17 mmol) in ethanol (0.5 mL), EtOAc (1 mL), water (0.5 mL) was added NaHSO₃ (36 mg, 0.35 mmol) at RT and heated to 50° C. for 16 h. The progress of the reaction was monitored by TLC and LCMS. After 16 h, the reaction mixture was cooled to RT and filtered through celite pad then washed with ethanol (5 mL). Filtrate was evaporated under reduce pressure to afford crude residue. This residue was triturated with di ethyl ether, EtOAc for 3 times to afford sodium (2S)-2-((2S)-2-(((2-(3-chlorophenyl)-1-phenylethoxy)carbonyl)amino)-3-cyclohexylpropanamido)-1-hydroxy-3-((S)-2-oxopyrrolidin-3-yl)propane-1-sulfonate (Compound C115). TLC system: 15% Methanol in dichloromethane Rf: 0.1 LCMS (ESI): m/z 648.2 [M−Na]⁻

### Example 85: Synthesis of Compounds C110 and C113

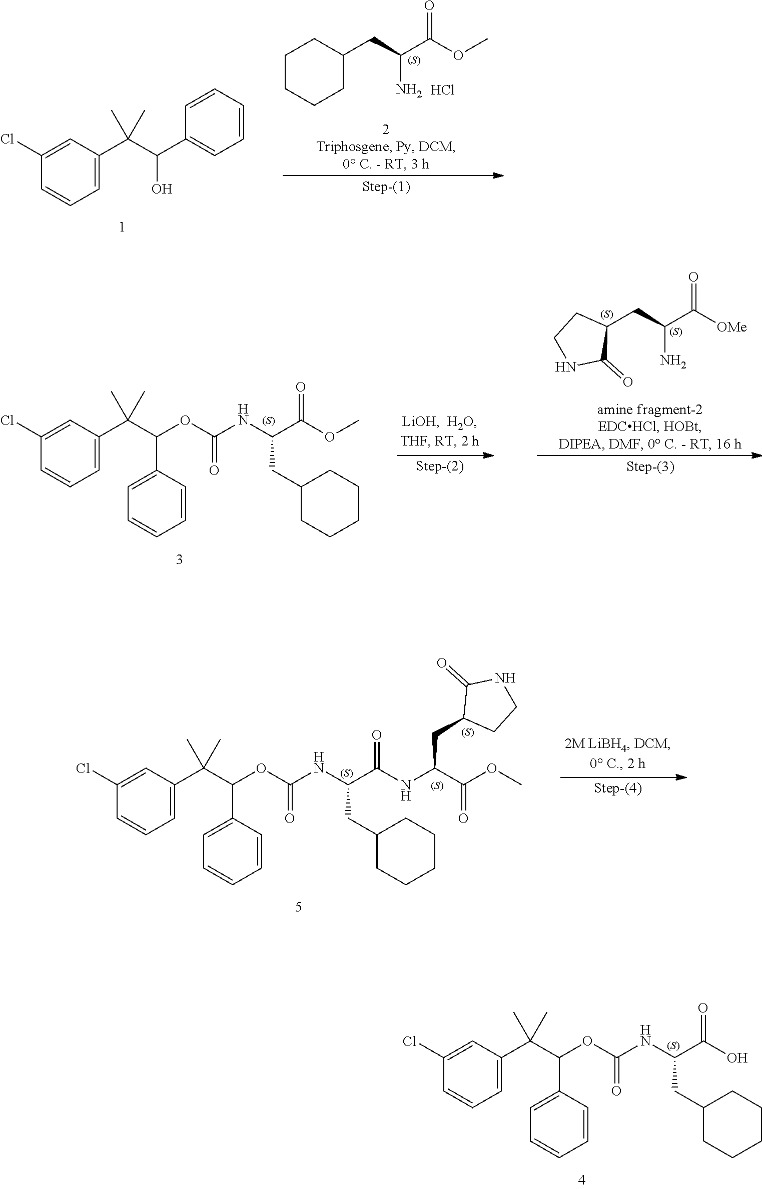

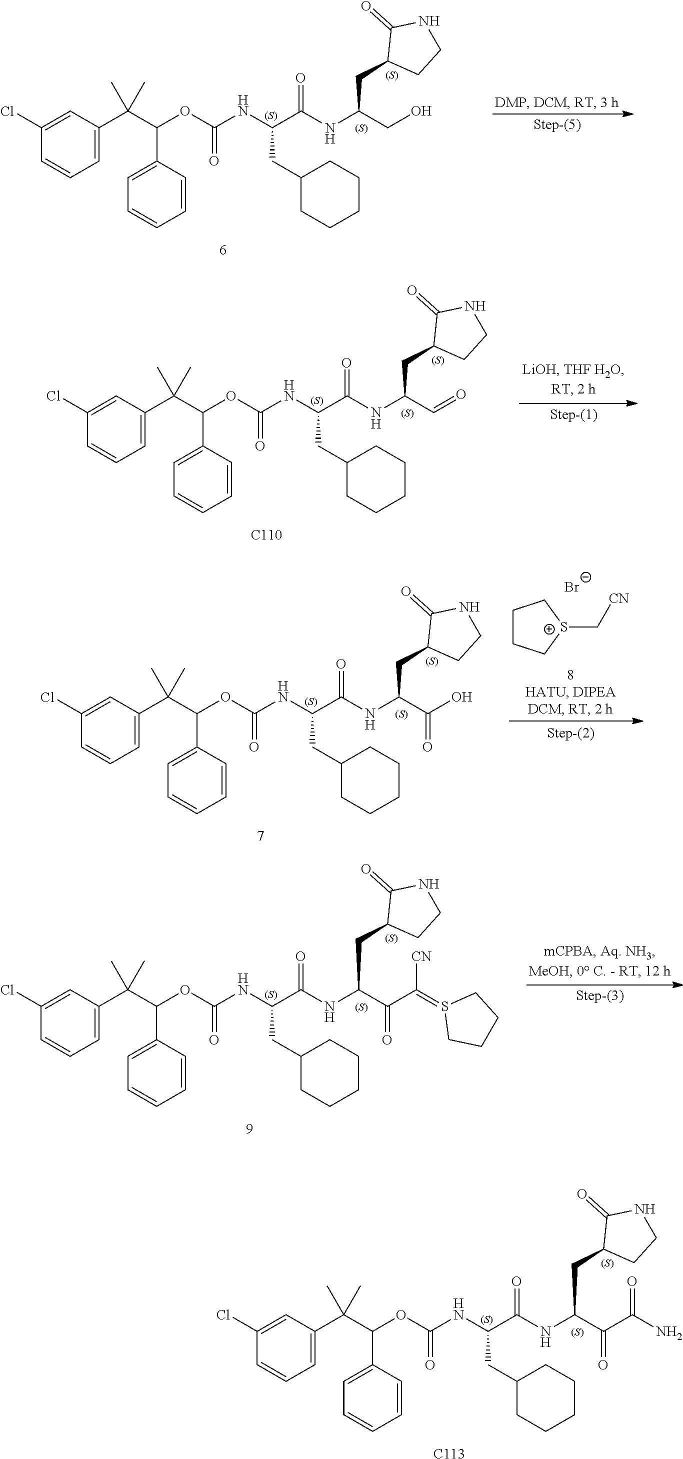

### Methyl (2S)-2-(((2-(3-chlorophenyl)-2-methyl-1-phenylpropoxy) carbonyl) amino)-3-cyclohexylpropanoate (3)

To a stirred solution of 2-(3-chlorophenyl)-2-methyl-1-phenylpropan-1-ol (1) (3.0 g, 11.53 mmol), methyl (S)-2-amino-3-cyclohexylpropanoate hydrochloride (2.5 g, 13.84 mmol) in DCM (40 mL) was added pyridine (9 mL, 3 vol) followed by triphosgene (1.7 g, 5.76 mmol) at 0° C. and stirred at room temperature for 3 h. The progress of the reaction was monitored by TLC and LCMS. Reaction mixture was quenched with 2N HCl (50 mL), extracted with DCM (2×40 mL). The combined organic layer was dried over sodium sulfate, filtered and evaporated under reduced pressure. The crude residue was purified by combi-flash, compound eluted at 8% ethyl acetate in pet ether to afford methyl (2S)-2-(((2-(3-chlorophenyl)-2-methyl-1-phenylpropoxy) carbonyl) amino)-3-cyclohexyl propanoate (3). TLC system: 10% Ethyl acetate in hexane Rf: 0.2 LCMS (ESI): m/z 494.15 [M+Na]

### (2S)-2-(((2-(3-chlorophenyl)-2-methyl-1-phenylpropoxy) carbonyl) amino)-3-cyclohexylpropanoic acid (4)

To a stirred solution of methyl (2S)-2-(((2-(3-chlorophenyl)-2-methyl-1-phenylpropoxy) carbonyl) amino)-3-cyclohexylpropanoate (3) (3.0 g, 6.36 mmol) in THF (20 mL), water (20 mL) was added lithium hydroxide (0.78 g, 19.10 mmol) at 0° C. and stirred at room temperature for 2 h. The progress of the reaction was monitored by TLC and LCMS. Reaction mixture completely distilled under reduced pressure, crude compound acidified with aq. 1N HCl solution up to pH˜3 and extracted with ethyl acetate (2×40 mL), dried over sodium sulfate, concentrated under reduced pressure to afford (2S)-2-(((2-(3-chlorophenyl)-2-methyl-1-phenylpropoxy) carbonyl) amino)-3-cyclohexylpropanoic acid (4). TLC system: 100% EtOAc Rf: 0.1 LCMS (ESI): m/z 456.17 [M−H]⁺

### Methyl (2S)-2-((2S)-2-(((2-(3-chlorophenyl)-2-methyl-1-phenylpropoxy) carbonyl) amino)-3-cyclohexylpropanamido)-3-((S)-2-oxopyrrolidin-3-yl) propanoate (5)

To a stirred solution of (2S)-2-(((2-(3-chlorophenyl)-2-methyl-1-phenyl propoxy)carbonyl)amino)-3-cyclohexylpropanoic acid (4) (2.8 g, 6.12 mmol) DMF (20 mL) added EDC.HCl (1.75 g, 9.19 mmol), HOBt (1.24 g, 9.19 mmol), DIPEA (3.2 mL, 18.38 mmol) and methyl (S)-2-amino-3-((S)-2-oxopyrrolidin-3-yl) propanoate hydrochloride (amine fragment-2) (1.36 g, 7.35 mmol) at 0° C. simultaneously and stirred at room temperature for 16 h. Reaction mixture was quenched with ice water (100 mL), extracted with ethyl acetate (2×50 mL), dried over sodium sulfate and evaporated under reduced pressure. The crude residue was purified by combi-flash NP, compound eluted at 4% methanol in dichloromethane to afford methyl(2S)-2-((2S)-2-(((2-(3-chlorophenyl)-2-methyl-1-phenylpropoxy)carbonyl)amino)-3-cyclohexylpropanamido)-3-((S)-2-oxopyrrolidin-3-yl) propanoate (5). TLC system: 10% Methanol in dichloromethane Rf: 0.4 LCMS (ESI): m/z 626.64 [M+H]⁺

### 2-(3-Chlorophenyl)-2-methyl-1-phenylpropyl ((S)-3-cyclohexyl-1-(((S)-1-hydroxy-3-((S)-2-oxopyrrolidin-3-yl) propan-2-yl) amino)-1-oxopropan-2-yl) carbamate (6)

To a stirred solution of methyl (2S)-2-((2S)-2-(((2-(3-chlorophenyl)-2-methyl-1-phenylpropoxy)carbonyl)amino)-3-cyclohexylpropanamido)-3-((S)-2-oxopyrrolidin-3-yl) propanoate (5) (2.0 g, 3.20 mmol) in DCM (20 mL) was added 2M LiBH₄ in THF (6.4 mL, 6.4 mmol) at 0° C. and the reaction mixture stirred for 2 h at 0° C. The progress of the reaction was monitored by TLC and LCMS. Reaction mixture was quenched with sat. Ammonium chloride solution (30 mL) and extracted with DCM (2×30 mL). Organic layer was washed with brine solution (30 mL), dried over Na₂SO₄ and concentrated to afford 2-(3-chlorophenyl)-2-methyl-1-phenylpropyl((S)-3-cyclohexyl-1-(((S)-1-hydroxy-3-((S)-2-oxopyrrolidin-3-yl)propan-2-yl) amino)-1-oxopropan-2-yl) carbamate (6). TLC system: 10% Methanol in dichloromethane Rf: 0.2 LCMS (ESI): m/z 598.67 [M+H]⁺

### 2-(3-Chlorophenyl)-2-methyl-1-phenylpropyl ((S)-3-cyclohexyl-1-oxo-1-(((S)-1-oxo-3-((S)-2-oxopyrrolidin-3-yl) propan-2-yl) amino) propan-2-yl) carbamate (Compound C110)

To a stirred solution of 2-(3-chlorophenyl)-2-methyl-1-phenylpropyl((S)-3-cyclohexyl-1-(((S)-1-hydroxy-3-((S)-2-oxopyrrolidin-3-yl)propan-2-yl) amino)-1-oxopropan-2-yl) carbamate (6) (1 g, 1.67 mmol) was dissolved in dichloromethane (20 mL) was added Dess-Martin periodinane (2.12 g, 5.01 mmol) at 0° C. and stirred at RT for 3 h. Reaction mixture was diluted with DCM (30 mL) and washed with sat. Hypo solution (3×30 mL) followed by sat. NaHCO₃ solution (3×30 mL). Organic layer was dried over anhydrous Na₂SO₄, filtered and concentrated to get crude compound. The crude compound was purified by prep HPLC to afford 2-(3-chlorophenyl)-2-methyl-1-phenylpropyl((S)-3-cyclohexyl-1-oxo-1-(((S)-1-oxo-3-((S)-2-oxopyrrolidin-3-yl) propan-2-yl) amino)propan-2-yl) carbamate (Compound C110). TLC system: 10% Methanol in dichloromethane Rf: 0.5 LCMS (ESI): m/z 596.3 (M+H)⁺

### (2S)-2-((2S)-2-(((2-(3-Chlorophenyl)-2-methyl-1-phenylpropoxy) carbonyl) amino)-3-cyclohexylpropanamido)-3-((S)-2-oxopyrrolidin-3-yl) propanoic acid (7)

To a stirred solution of methyl (2S)-2-((2S)-2-(((2-(3-chlorophenyl)-2-methyl-1-phenylpropoxy)carbonyl)amino)-3-cyclohexylpropanamido)-3-((S)-2-oxopyrrolidin-3-yl)propanoate (5) (500 mg, 0.80 mmol) in THF (6 mL), water (3 mL) was added lithium hydroxide (100 mg, 2.4 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-((2S)-2-(((2-(3-chlorophenyl)-2-methyl-1-phenylpropoxy)carbonyl)amino)-3-cyclohexylpropanamido)-3-((S)-2-oxopyrrolidin-3-yl)propanoic acid (7). TLC system: 10% MeOH in DCM Rf: 0.2 LCMS (ESI): m/z 610.37 [M−H]⁺

### 2-(3-Chlorophenyl)-2-methyl-1-phenylpropyl ((S)-1-(((S)-4-cyano-3-oxo-1-((S)-2-oxopyrrolidin-3-yl)-4-(tetrahydro-1l4-thiophen-1-ylidene) butan-2-yl) amino)-3-cyclohexyl-1-oxopropan-2-yl) carbamate (9)

To a stirred solution of (2S)-2-((2S)-2-(((2-(3-chlorophenyl)-2-methyl-1-phenylpropoxy)carbonyl)amino)-3-cyclohexylpropanamido)-3-((S)-2-oxopyrrolidin-3-yl)propanoic acid (7) (600 mg, 0.98 mmol) DCM (15 mL) added HATU (746 mg, 1.96 mmol), DIPEA (5.4 mL, 2.94 mmol) and 1-(cyanomethyl)tetrahydro-1H-thiophen-1-iumbromide (8) (242 mg, 1.17 mmol) at 0° C. simultaneously and stirred at room temperature for 2 h. Reaction mixture was quenched with ice water (20 mL), extracted with dichloromethane (2×15 mL), dried over sodium sulfate and evaporated under reduced pressure. The crude residue was purified by combi-flash NP, compound eluted at 5% methanol in dichloromethane to afford 2-(3-chlorophenyl)-2-methyl-1-phenylpropyl ((S)-1-(((S)-4-cyano-3-oxo-1-((S)-2-oxopyrrolidin-3-yl)-4-(tetrahydro-1l4-thiophen-1-ylidene) butan-2-yl) amino)-3-cyclohexyl-1-oxopropan-2-yl)carbamate (9). TLC system: 10% Methanol in dichloromethane Rf: 0.4 LCMS (ESI): m/z 721.69 [M+H]⁺

### 2-(3-Chlorophenyl)-2-methyl-1-phenylpropyl ((S)-1-(((S)-4-amino-3,4-dioxo-1-((S)-2-oxopyrrolidin-3-yl) butan-2-yl) amino)-3-cyclohexyl-1-oxopropan-2-yl) carbamate (Compound C113)

To a stirred solution of 2-(3-chlorophenyl)-2-methyl-1-phenylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl) carbamate (9) (350 mg, 0.47 mmol) in methanol (5 mL) was added m-CPBA (162 mg, 0.94 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×20 mL). Organic layer was washed with brine solution (30 mL), dried over Na₂SO₄ and concentrated to get crude compound. The crude compound was purified by prep HPLC to afford 2-(3-chlorophenyl)-2-methyl-1-phenylpropyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C113). TLC system: 10% Methanol in dichloromethane Rf: 0.2 LCMS (ESI): m/z 521.2 [M+H]⁺

### Example 86: Synthesis of Compound C105

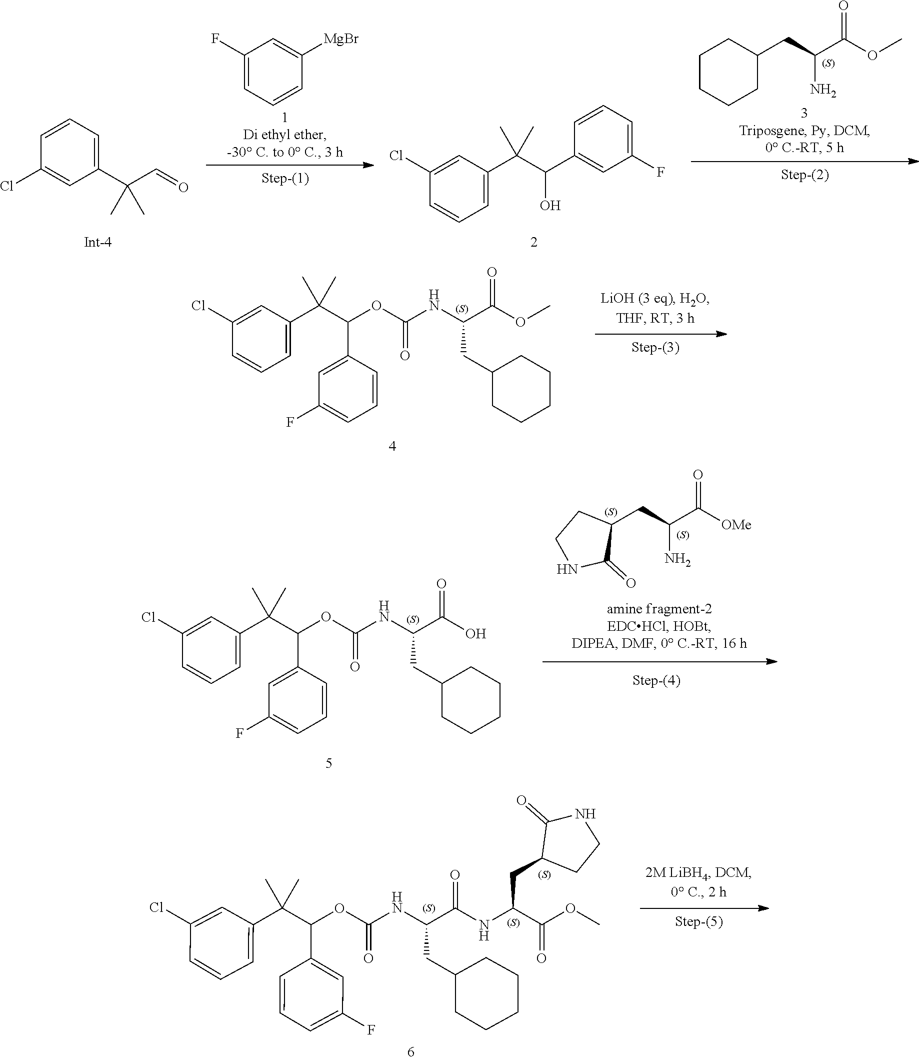

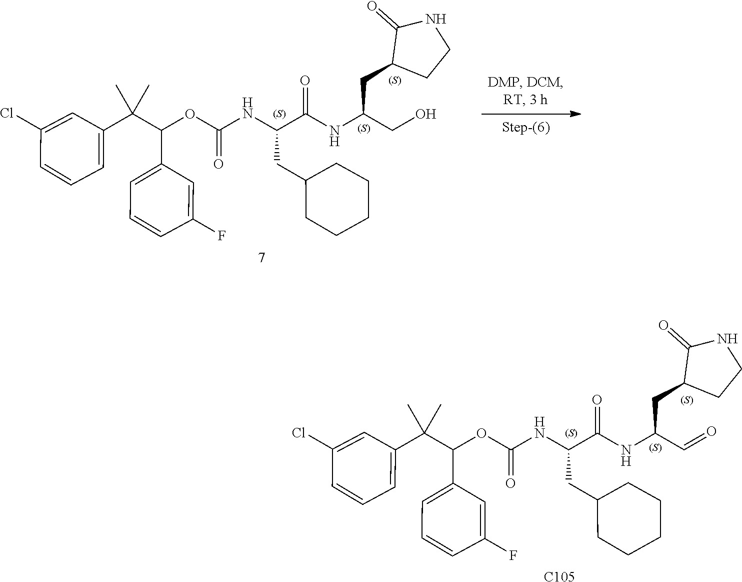

### 2-(3-chlorophenyl)-1-(3-fluorophenyl)-2-methylpropan-1-ol (2)

To a stirred solution of (3-fluorophenyl) magnesium bromide (1) (32 mL, 65.934 mmol) was added 2-(3-chlorophenyl)-2-methylpropanal (Int-4) (4 g, 21.978 mmol) in diethyl ether (50 mL) at −30° C. and stirred at RT for 3 h. The progress of the reaction was monitored by TLC. Reaction mixture was quenched with saturated ammonium chloride solution and extracted with ethyl acetate (2×100 mL). Combined organic layer and washed with water (2×50 mL), dried over sodium sulfate, concentrated under reduced pressure to afford crude residue, crude residue was purified by normal phase chromatography to afford 2-(3-chlorophenyl)-1-(3-fluorophenyl)-2-methylpropan-1-ol (2). TLC system: 5% Ethyl acetate in hexane Rf: 0.7 LCMS (ESI): m/z 261.27 [M−OH]⁻

### Methyl (2S)-2-(((2-(3-chlorophenyl)-1-(3-fluorophenyl)-2-methylpropoxy)carbonyl)amino)-3-cyclohexylpropanoate (4)

To a stirred solution of 2-(3-chlorophenyl)-1-(3-fluorophenyl)-2-methylpropan-1-ol (2) (4.0 g, 14.388 mmol) in DCM (40 mL) ware added pyridine (4.0 mL, 1 vol) and methyl (S)-2-amino-3-cyclohexylpropanoate (3) (3.2 g, 17.266) then added triphosgene (2.12 g, 7.194 mmol) portion wise for 15 min at 0° C. and stirred at room temperature for 5 h. The progress of the reaction was monitored by TLC and LCMS. Reaction mixture was diluted with DCM and washed with 1N HCl (50 mL), organic layer was dried over sodium sulfate, filtered and evaporated under reduced pressure. The crude residue was purified by combi-flash, compound eluted at 10% ethyl acetate in pet ether to afford methyl (2S)-2-(((2-(3-chlorophenyl)-1-(3-fluorophenyl)-2-methylpropoxy)carbonyl)amino)-3-cyclohexylpropanoate (4). TLC system: 5% Ethyl acetate in hexane Rf: 0.3 LCMS (ESI): m/z 512.19 [M+Na]⁺

### (2S)-2-(((2-(3-chlorophenyl)-1-(3-fluorophenyl)-2-methylpropoxy)carbonyl)amino)-3-cyclohexylpropanoic acid (5)

To a stirred solution of methyl (2S)-2-(((2-(3-chlorophenyl)-1-(3-fluorophenyl)-2-methylpropoxy)carbonyl)amino)-3-cyclohexylpropanoate (4) (3.5 g, 7.157 mmol) in THF (25 mL), water (15 mL) was added lithium hydroxide (515 mg, 21.472 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-(((2-(3-chlorophenyl)-1-(3-fluorophenyl)-2-methylpropoxy)carbonyl)amino)-3-cyclohexylpropanoic acid (5). TLC system: 30% Ethyl acetate in hexane Rf: 0.2 LCMS (ESI): m/z 498.3 [M+Na]⁺

### Methyl (2S)-2-((2S)-2-(((2-(3-chlorophenyl)-1-(3-fluorophenyl)-2-methylpropoxy)carbonyl)amino)-3-cyclohexylpropanamido)-3-((S)-2-oxopyrrolidin-3-yl)propanoate (6)

To a stirred solution of (2S)-2-(((2-(3-chlorophenyl)-1-(3-fluorophenyl)-2-methylpropoxy)carbonyl)amino)-3-cyclohexylpropanoic acid (5) (2 g, 4.2105 mmol) DMF (20 mL) added EDC.HCl (1.2 g, 6.315 mmol), HOBt (852 mg, 6.315 mmol), DIPEA (2.3 mL, 12.631 mmol) and methyl (S)-2-amino-3-((S)-2-oxopyrrolidin-3-yl)propanoate hydrochloride (amine fragment-2) (940 mg, 5.052 mmol) at 0° C. simultaneously and stirred at room temperature for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 30% Ethyl acetate in pet ether to afford methyl (2S)-2-((2S)-2-(((2-(3-chlorophenyl)-1-(3-fluorophenyl)-2-methylpropoxy)carbonyl)amino)-3-cyclohexylpropanamido)-3-((S)-2-oxopyrrolidin-3-yl)propanoate (6). TLC system: 80% Ethyl acetate in Pet ether Rf: 0.5 LCMS (ESI): m/z 645.11 [M+H]⁺

### 2-(3-Chlorophenyl)-1-(3-fluorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (7)

To a stirred solution of methyl (2S)-2-((2S)-2-(((2-(3-chlorophenyl)-1-(3-fluorophenyl)-2-methylpropoxy)carbonyl)amino)-3-cyclohexylpropanamido)-3-((S)-2-oxopyrrolidin-3-yl)propanoate (6) (1.8 g, 2.7993 mmol) in DCM (20 mL) was added 2M LiBH₄ in THF (3.5 mL, 6.998 mmol) at 0° C. and the reaction mixture stirred for 2 h at 0° C. The progress of the reaction was monitored by TLC and LCMS. Reaction mixture was quenched with saturated ammonium chloride solution (20 mL) and extracted with DCM (2×20 mL). Organic layer was washed with brine solution (30 mL), dried over Na₂SO₄ and concentrated to afford 2-(3-chlorophenyl)-1-(3-fluorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (7). TLC system: 10% Methanol in dichloromethane Rf: 0.2 LCMS (ESI): m/z 616, 73 [M+H]⁺

### 2-(3-Chlorophenyl)-1-(3-fluorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (Compound C105)

To a stirred solution of 2-(3-chlorophenyl)-1-(3-fluorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (7) (200 mg, 0.324 mmol) was dissolved in dichloromethane (5 mL) was added Dess-Martin periodinane (275 mg, 0.649 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford 2-(3-chlorophenyl)-1-(3-fluorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (Compound C105) TLC system: 10% Methanol in dichloromethane Rf: 0.5 LCMS (ESI): m/z 614.3 (M+H)⁺

### Example 87: Synthesis of Compound C106

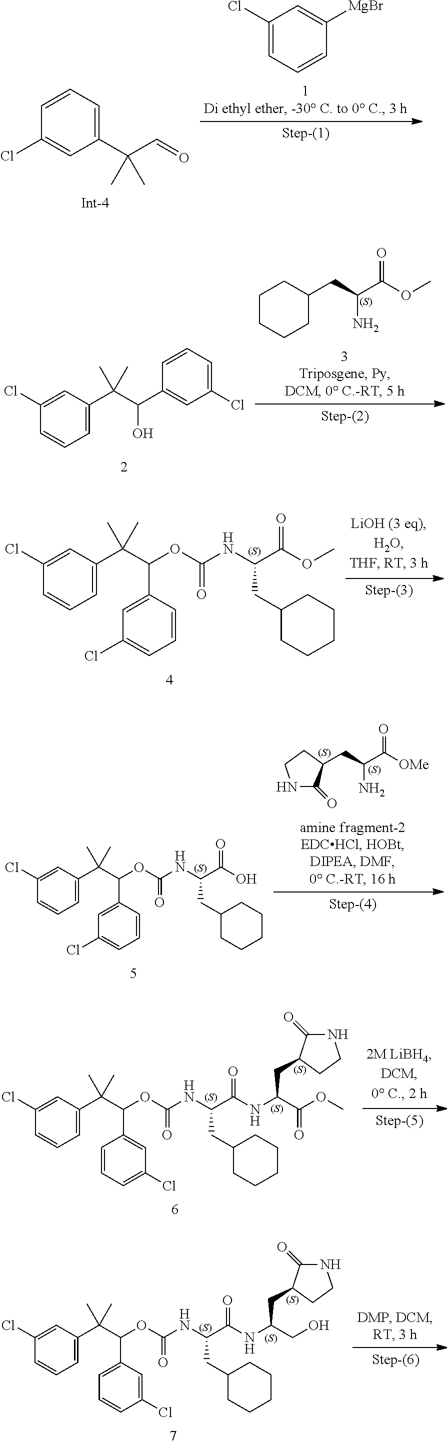

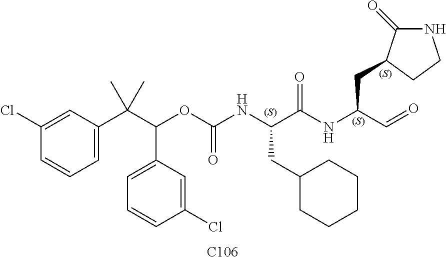

### 1,2-Bis(3-chlorophenyl)-2-methylpropan-1-ol (2)

To a stirred solution of (3-chlorophenyl) magnesium bromide in tetrahydrofuran (1) (41 mL, 27.472 mmol) was added 2-(3-chlorophenyl)-2-methylpropanal (5 g, 27.472 mmol) in di ethyl ether (50 mL) at −30° C. and stirred at RT for 3 h. The progress of the reaction was monitored by TLC. Reaction mixture was quenched with saturated ammonium chloride solution and extracted with ethyl acetate (2×100 mL). Combined organic layer and washed with water (2×50 mL), dried over sodium sulfate, concentrated under reduced pressure to afford crude residue, crude residue was purified by normal phase chromatography to afford 1,2-bis(3-chlorophenyl)-2-methylpropan-1-ol (2). TLC system: 5% Ethyl acetate in hexane Rf: 0.7 LCMS (ESI): m/z 277.16 [M−OH]⁻

### Methyl (2S)-2-(((1,2-bis(3-chlorophenyl)-2-methylpropoxy)carbonyl)amino)-3-cyclohexylpropanoate (4)

To a stirred solution of 1,2-bis(3-chlorophenyl)-2-methylpropan-1-ol (2) (4.2 g, 14.285 mmol) in DCM (40 mL) ware added pyridine (4.2 mL, 1 vol) and methyl (S)-2-amino-3-cyclohexylpropanoate (3) (3.17 g, 17.1428) then added triphosgene (2.11 g, 7.1428 mmol) portion wise for 15 min at 0° C. and stirred at room temperature for 5 h. The progress of the reaction was monitored by TLC and LCMS. Reaction mixture was diluted with DCM and washed with 1N HCl (50 mL), organic layer was dried over sodium sulfate, filtered and evaporated under reduced pressure. The crude residue was purified by combi-flash, compound eluted at 10% ethyl acetate in pet ether to afford methyl (2S)-2-(((1,2-bis(3-chlorophenyl)-2-methylpropoxy)carbonyl)amino)-3-cyclohexylpropanoate (4). TLC system: 5% Ethyl acetate in hexane Rf: 0.3 LCMS (ESI): m/z 528.24 [M+Na]⁺

### (2S)-2-(((1,2-bis(3-chlorophenyl)-2-methylpropoxy)carbonyl)amino)-3-cyclohexylpropanoic acid (5)

To a stirred solution of methyl (2S)-2-(((1,2-bis(3-chlorophenyl)-2-methylpropoxy)carbonyl)amino)-3-cyclohexylpropanoate (4) (3.2 g, 6.336 mmol) in THF (20 mL), water (15 mL) was added lithium hydroxide (456 mg, 19.009 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-(((1,2-bis(3-chlorophenyl)-2-methylpropoxy)carbonyl)amino)-3-cyclohexylpropanoic acid (5). TLC system: 30% Ethyl acetate in hexane Rf: 0.2 LCMS (ESI): m/z 514.22 [M+Na]⁺

### Methyl (2S)-2-((2S)-2-(((1,2-bis(3-chlorophenyl)-2-methylpropoxy)carbonyl)amino)-3-cyclohexylpropanamido)-3-((S)-2-oxopyrrolidin-3-yl)propanoate (6)

To a stirred solution of (2S)-2-(((1,2-bis(3-chlorophenyl)-2-methylpropoxy)carbonyl)amino)-3-cyclohexylpropanoic acid (5) (1.5 g, 3.054 mmol) DMF (15 mL) added EDC.HCl (875 mg, 4.582 mmol), HOBt (618 mg, 4.582 mmol), DIPEA (1.7 mL, 9.164 mmol) and methyl (S)-2-amino-3-((S)-2-oxopyrrolidin-3-yl)propanoate hydrochloride (amine fragment-2) (681 mg, 3.665 mmol) at 0° C. simultaneously and stirred at room temperature for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 30% Ethyl acetate in pet ether to afford methyl (2S)-2-((2S)-2-(((1,2-bis(3-chlorophenyl)-2-methylpropoxy)carbonyl)amino)-3-cyclohexylpropanamido)-3-((S)-2-oxopyrrolidin-3-yl)propanoate (6). TLC system: 80% Ethyl acetate in Pet ether Rf: 0.5 LCMS (ESI): m/z 683.78 [M+Na]⁺

### 1,2-Bis(3-chlorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (7)

To a stirred solution of methyl (2S)-2-((2S)-2-(((1,2-bis(3-chlorophenyl)-2-methylpropoxy)carbonyl)amino)-3-cyclohexylpropanamido)-3-((S)-2-oxopyrrolidin-3-yl)propanoate (6) (1 g, 1.517 mmol) in DCM (10 mL) was added 2M LiBH₄ in THF (1.5 mL, 3.034 mmol) at 0° C. and the reaction mixture stirred for 2 h at 0° C. The progress of the reaction was monitored by TLC and LCMS. Reaction mixture was quenched with saturated ammonium chloride solution (20 mL) and extracted with DCM (2×20 mL). Organic layer was washed with brine solution (30 mL), dried over Na₂SO₄ and concentrated to afford 1,2-bis(3-chlorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (7). TLC system: 10% Methanol in dichloromethane Rf: 0.2 LCMS (ESI): m/z 632.64 [M+H]⁺

### 1,2-Bis(3-chlorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (Compound C106)

To a stirred solution of 1,2-bis(3-chlorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (7) (200 mg, 0.316 mmol) was dissolved in dichloromethane (10 mL) was added Dess-Martin periodinane (268 mg, 0.633 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford 1,2-bis(3-chlorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (Compound C106). TLC system: 10% Methanol in dichloromethane Rf: 0.5 LCMS (ESI): m/z 630.2 (M+H)⁺

### Example 88: Synthesis of Compound C112

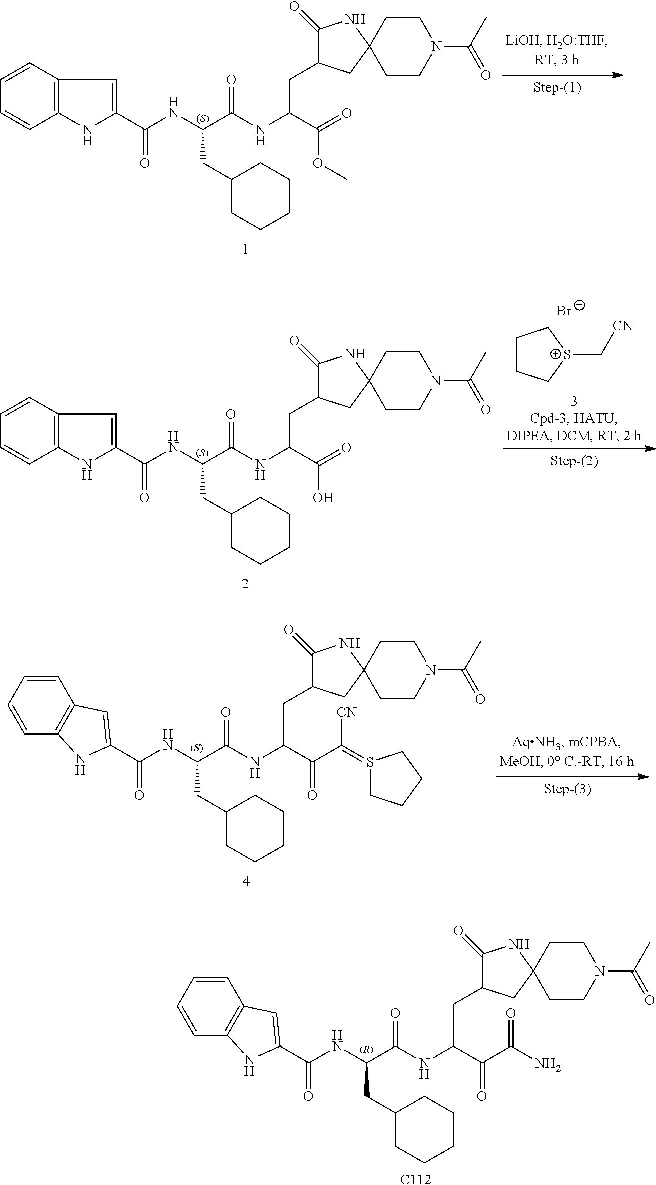

### 3-(8-Acetyl-2-oxo-1,8-diazaspiro[4.5]decan-3-yl)-2-((S)-3-cyclohexyl-2-(1H-indole-2-carboxamido)propanamido)propanoic acid (2)

To a stirred solution of methyl 3-(8-acetyl-2-oxo-1,8-diazaspiro[4.5]decan-3-yl)-2-((S)-3-cyclohexyl-2-(1H-indole-2-carboxamido)propanamido)propanoate (1) (900 mg, 1.51 mmol) in THF (7 mL), water (3 mL) was added lithium hydroxide (109 mg, 4.55 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×20 mL), dried over sodium sulfate, concentrated under reduced pressure to afford 3-(8-acetyl-2-oxo-1,8-diazaspiro[4.5]decan-3-yl)-2-((S)-3-cyclohexyl-2-(1H-indole-2-carboxamido)propanamido)propanoic acid (2) TLC system: 10% MeOH in DCM Rf: 0.2 LCMS (ESI): m/z 580.19 [M+H]⁺

### N-((2S)-1-((1-(8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-1H-indole-2-carboxamide (5)

To a stirred solution of 3-(8-acetyl-2-oxo-1,8-diazaspiro[4.5]decan-3-yl)-2-((S)-3-cyclohexyl-2-(1H-indole-2-carboxamido)propanamido)propanoic acid (2) (570 mg, 0.984 mmol) in THF (10 mL) added HATU (514 mg, 1.476 mmol), DIPEA (0.51 mL, 2.215 mmol) and 1-(cyanomethyl)tetrahydro-1H-thiophen-1-ium (3) (304 mg, 1.476 mmol) at 0° C. simultaneously and stirred at room temperature for 2 h. Reaction mixture was diluted with ice water (50 mL), extracted with EtOAc (2×20 mL), dried over sodium sulfate and evaporated under reduced pressure. The crude residue was purified by combi-flash NP, compound eluted at 5% methanol in dichloromethane to afford N-((2S)-1-((1-(8-acetyl-2-oxo-1,8-di azaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-1H-indole-2-carboxamide (4). TLC system: 10% Methanol in dichloromethane Rf: 0.4 LCMS (ESI): m/z 689.61 [M+H]⁺

### N-((2R)-1-((1-(8-acetyl-2-oxo-1,8-diazaspiro[4.5]decan-3-yl)-4-amino-3,4-dioxobutan-2-yl)amino)-3-cyclohexyl-1-oxopropan-2-yl)-1H-indole-2-carboxamide (Compound C112)

To a stirred solution of N-((2S)-1-((1-(8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-1H-indole-2-carboxamide (4) (200 mg, 0.290 mmol) in methanol (3 mL) was added m-CPBA (124 mg, 0.726 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep-HPLC to afford N-((2R)-1-((1-(8-acetyl-2-oxo-1,8-diazaspiro[4.5]decan-3-yl)-4-amino-3,4-dioxobutan-2-yl)amino)-3-cyclohexyl-1-oxopropan-2-yl)-1H-indole-2-carboxamide (Compound C112). TLC system: 10% Methanol in dichloromethane Rf: 0.3 LCMS (ESI): m/z 607.3 [M+H]⁺

### Example 89: Synthesis of Compound C116

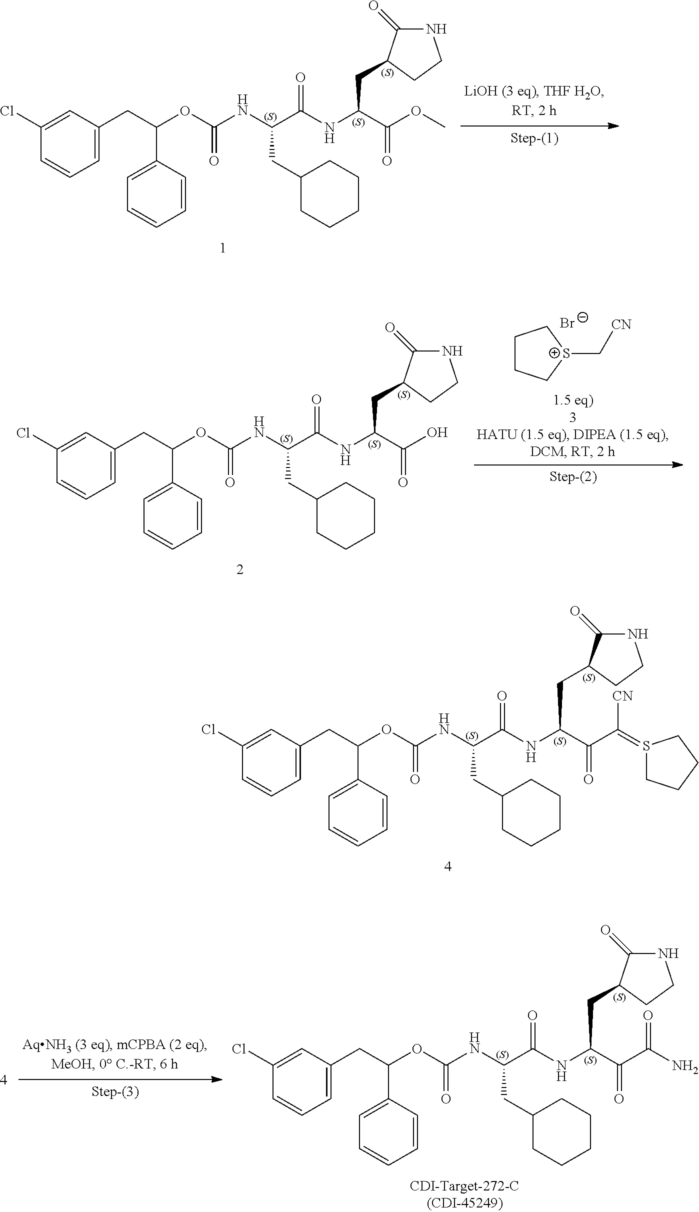

### (2S)-2-((2S)-2-(((2-(3-chlorophenyl)-1-phenylethoxy)carbonyl)amino)-3-cyclohexylpropanamido)-3-((S)-2-oxopyrrolidin-3-yl)propanoic acid (2)

To a stirred solution of methyl (2S)-2-((2S)-2-(((2-(3-chlorophenyl)-1-phenylethoxy)carbonyl)amino)-3-cyclohexylpropanamido)-3-((S)-2-oxopyrrolidin-3-yl)propanoate (1) (1 g, 1.67 mmol) in THF (15 mL), water (5 mL) was added lithium hydroxide (206 mg, 5.02 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford (2S)-2-(((2-(3-chlorophenyl)-1-phenylethoxy)carbonyl)amino)-3-cyclohexylpropanoic acid (2). TLC system: 100% EtOAc Rf: 0.1 LCMS (ESI): m/z 584.37 [M+H]⁺

### 2-(3-Chlorophenyl)-1-phenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (4)

To a stirred solution of (2S)-2-((2S)-2-(((2-(3-chlorophenyl)-1-phenylethoxy)carbonyl)amino)-3-cyclohexylpropanamido)-3-((S)-2-oxopyrrolidin-3-yl)propanoic acid (2) (500 mg, 0.85 mmol) DCM (5 mL) added HATU (488 mg, 1.28 mmol), DIPEA (0.4 mL, 9.12 mmol) and 1-(cyanomethyl)tetrahydro-1H-thiophen-1-iumbromide (3) (265 mg, 1.28 mmol) at 0° C. simultaneously and stirred at room temperature for 2 h. Reaction mixture was diluted with ice water (50 mL), extracted with dichloromethane (2×60 mL), dried over sodium sulfate and evaporated under reduced pressure. The crude residue was purified by combi-flash NP, compound eluted at 5% methanol in dichloromethane to afford 1-(3-chlorobenzyl)cyclopropyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4). TLC system: 10% Methanol in dichloromethane Rf: 0.5 LCMS (ESI): m/z 693.3 (M+H)⁺

### 2-(3-Chlorophenyl)-1-phenylethyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C116)

To a stirred solution of 2-(3-chlorophenyl)-1-phenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (4) (180 mg, 0.26 mmol) in methanol (2 mL) was added mCPBA (89 mg, 0.52 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford 2-(3-chlorophenyl)-1-phenylethyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C116). TLC system: 15% Methanol in dichloromethane Rf: 0.1 LCMS (ESI): m/z 611.3 [M+H]⁺

### Example 90: Synthesis of Compounds C118

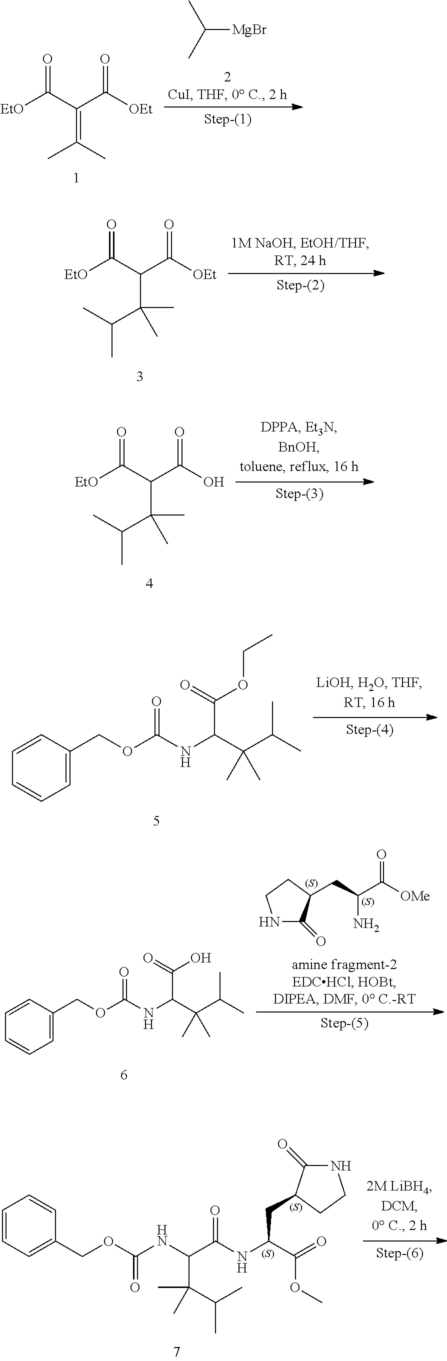

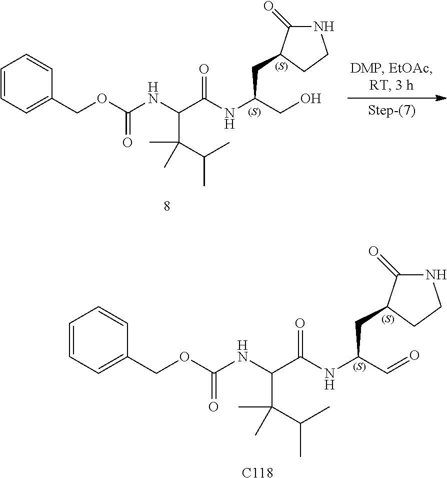

### Diethyl 2-(2,3-dimethylbutan-2-yl)malonate (3)

A solution of diethyl 2-(propan-2-ylidene)malonate (1) (5 g, 25.00 mmol), in THF (100 mL) was cooled to 0° C., followed by copper (I) iodide (7.1 g, 37.00 mmol). The mixture was stirred at 0° C. for 0.5 h. Then isopropyl magnesium bromide 1.5M in THF (50 mL, 75.00 mmol) was added drop-wise into the above mixture at 0° C. The mixture was stirred at 0° C. for 2 h. Progress of the reaction was monitored by TLC and LCMS. The mixture was quenched with 1N HCl and extracted with ethyl acetate (2×100 mL) and washed with water (2×100 mL), dried over sodium sulfate, concentrated under reduced pressure to afford diethyl 2-(2,3-dimethylbutan-2-yl)malonate (3). TLC system: 5% Ethyl acetate in Pet ether R_{f}: 0.6 LCMS (ESI): m/z 245.39 [M+H]⁺

### 2-(Ethoxycarbonyl)-3,3,4-trimethylpentanoic acid (4)

A stirred solution of (3) (6 g, 24.59 mmol) in mixture of ethanol (150 mL) and THF (75 mL), was treated with 1N solution of NaOH (25 mL, 24.59 mmol) and the reaction mixture was stirred at room temperature for 24 h, After that the mixture was evaporated to syrup and dissolved in water (200 mL) and extracted with ethyl ether (2×100 mL). The aqueous phase was acidified with 1N HCl to pH 2.0 and extracted with EtOAc (2×100 mL). The combined organic layers were washed with brine solution (200 mL), dried over Na₂SO₄ and concentrated to afford 2-(ethoxycarbonyl)-3,3,4-trimethylpentanoic acid (4). TLC system: 50% EtOAc in Pet ether Rf: 0.1 LCMS (ESI): m/z 217.08 [M+H]⁺

### Ethyl 2-(((benzyloxy)carbonyl)amino)-3,3,4-trimethylpentanoate (5)

To a stirred solution of (4) (2 g, 9.259 mmol) in dry benzene, triethylamine (2.62 mL, 18.518 mmol) and diphenylphosphoryl azide (2.98 mL, 13.888 mmol) were added. The reaction mixture was heated at reflux for 2 h. After cooling to room temperature, benzyl alcohol (1.5 mL, 13.88 mmol) was added and the reaction was heated again at reflux for 16 h. After evaporation of the solvent, the crude material was quenched with 5% citric acid solution and extracted with ethyl acetate (2×50 mL). The combined organic layers were washed with brine solution (50 mL), dried over Na₂SO₄ and concentrated and the crude was purified by flash chromatography to afford ethyl 2-(((benzyloxy)carbonyl)amino)-3,3,4-trimethylpentanoate (5). TLC system: 50% EtOAc in Pet ether Rf: 0.5 LCMS (ESI): m/z 322.44 [M+H]⁺

### 2-(((benzyloxy)carbonyl)amino)-3,3,4-trimethylpentanoic acid (6)

To a stirred solution of methyl ethyl 2-(((benzyloxy)carbonyl)amino)-3,3,4-trimethylpentanoate (5) (2 g, 6.230 mmol) in MeOH/THF (15 mL), water (5 mL) was added lithium hydroxide (785 mg, 18.691 mmol) at RT and stirred at room temperature for 16 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), The combined organic layers were washed with brine solution (20 mL), dried over Na₂SO₄ and concentrated to afford 2-(((benzyloxy)carbonyl)amino)-3,3,4-trimethylpentanoic acid (6). TLC system: 10% MeOH in DCM Rf: 0.1 LCMS (ESI): m/z 294.37 [M+H]⁺

### Methyl (2S)-2-(2-(((benzyloxy)carbonyl)amino)-3,3,4-trimethylpentanamido)-3-((S)-2-oxopyrrolidin-3-yl)propanoate (7)

To a stirred solution of 2-(((benzyloxy)carbonyl)amino)-3,3,4-trimethylpentanoic acid (6) (1.5 g, 5.119 mmol) in DMF (15 mL) was added EDC.HCl (1.46 g, 7.679 mmol), HOBt (1.03 g, 7.67 mmol), and DIPEA (2.67 mL, 15.358 mmol) at 0° C., then methyl 2-amino-2-methyl-3-(2-oxopyrrolidin-3-yl)propanoate hydrochloride (amine fragment-2A) (1.25 g, 5.6313 mmol) at 0° C. and stirred at room temperature for 16 h. Reaction mixture was diluted with ice water (50 mL), extracted with ethyl acetate (2×50 mL), dried over sodium sulfate and evaporated under reduced pressure. The crude was purified by flash chromatography to afford methyl (2S)-2-(2-(((benzyloxy)carbonyl)amino)-3,3,4-trimethylpentanamido)-3-((S)-2-oxopyrrolidin-3-yl)propanoate (7). TLC system: 5% MeOH in DCM Rf: 0.3 LCMS (ESI): m/z 462.53 [M+H]⁺

### Benzyl (1-(((S)-1-hydroxy-3-((S)-2-oxopyrrolidin-3-yl)propan-2-yl)amino)-3,3,4-trimethyl-1-oxopentan-2-yl)carbamate (8)

To a stirred solution of methyl (2S)-2-(2-(((benzyloxy)carbonyl)amino)-3,3,4-trimethylpentanamido)-3-((S)-2-oxopyrrolidin-3-yl)propanoate (7) (500 mg, 1.084 mmol) in DCM (5 mL) was added 2M LiBH₄ in THF (1.08 mL, 2.16 mmol) at 0° C. and the reaction mixture stirred for 2 h at 0° C. The progress of the reaction was monitored by TLC and LCMS. Reaction mixture was quenched with sat. ammonium chloride solution (30 mL) and extracted with DCM (2×30 mL). Organic layer was washed with brine solution (20 mL), dried over Na₂SO₄ and concentrated to afford benzyl (1-(((S)-1-hydroxy-3-((S)-2-oxopyrrolidin-3-yl)propan-2-yl)amino)-3,3,4-trimethyl-1-oxopentan-2-yl)carbamate (8). TLC system: 10% MeOH in DCM Rf: 0.4 LCMS (ESI): m/z 434.20 [M+H]⁺

### Benzyl (3,3,4-trimethyl-1-oxo-1-(((S)-1-oxo-3-((S)-2-oxopyrrolidin-3-yl)propan-2-yl)amino)pentan-2-yl)carbamate (Compound C118)

To a stirred solution of (1-(((S)-1-hydroxy-3-((S)-2-oxopyrrolidin-3-yl)propan-2-yl)amino)-3,3,4-trimethyl-1-oxopentan-2-yl)carbamate (8) (200 mg, 0.461 mmol) in ethyl acetate (5 mL) was added Dess-Martin periodinane (293 mg, 0.692 mmol) at 0° C. and stirred at RT for 16 h. The progress of the reaction was monitored by TLC and LCMS. After complete consumption of the starting material by TLC and LCMS, the reaction mass was filtered through celite pad and the celite pad thoroughly was with ethyl acetate (30 mL). Then the organic layer was washed with 10% sodium thiosulfate solution (2×50 mL) followed by saturated sodium bicarbonate solution (2×50 mL), water (1×50 mL), brine 1×50 mL). Then the organic layer was dried over sodium sulfate and evaporated under vacuum. Then the crude compound was purified by Prep-HPLC purification to get benzyl (3,3,4-trimethyl-1-oxo-1-(((S)-1-oxo-3-((S)-2-oxopyrrolidin-3-yl)propan-2-yl)amino)pentan-2-yl)carbamate (Compound C118). TLC system: 10% MeOH in DCM Rf: 0.45 LCMS (ESI): m/z 432.2 (M+H)⁺

### Example 91: Synthesis of Compound C119

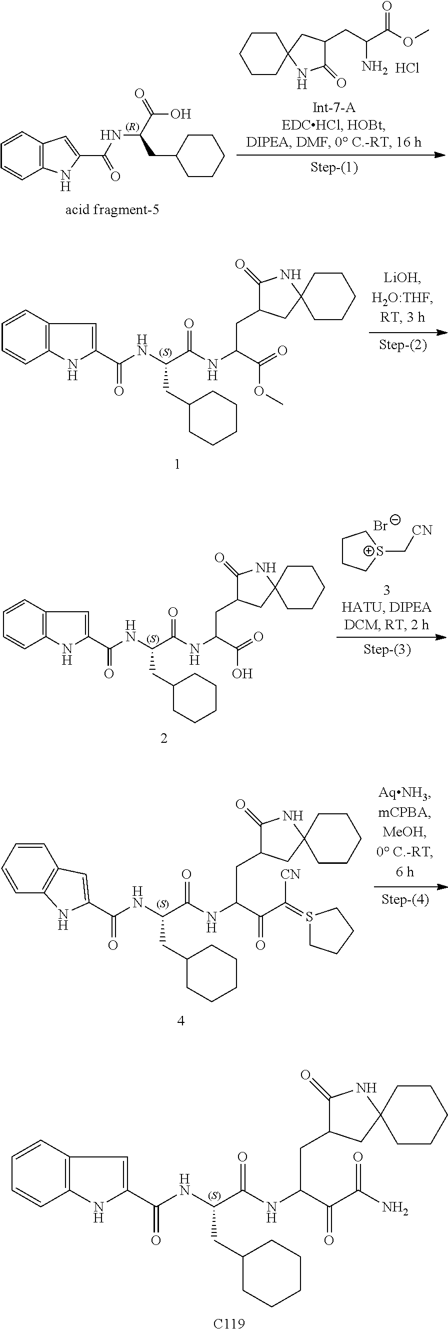

### Methyl 2-((S)-3-cyclohexyl-2-(1H-indole-2-carboxamido)propanamido)-3-(2-oxo-1-azaspiro[4.5]decan-3-yl)propanoate (1)

To a stirred solution (S)-3-cyclohexyl-2-(1H-indole-2-carboxamido)propanoic acid (Acid fragment-5) (1 g, 2.564 mmol) in DMF (15 mL) at 0° C. was added EDC.HCl (842 mg, 4.424 mmol), HOBT (590 g, 4.424 mmol), DIPEA (1.9 mL, 10 mmol) and methyl 2-amino-3-(2-oxo-1-azaspiro[4.5]decan-3-yl)propanoate hydrochloride (Int-7A) (940 mg, 3.244 mmol) simultaneously and stirred at room temperature for 16 h. The progress of the reaction was monitored by TLC and LCMS. After 16 h, reaction mixture was quenched with ice water (50 mL), extracted with ethyl acetate (2×100 mL), the combined organic layer was dried over sodium sulfate and evaporated under reduced pressure. The crude residue was purified by silica gel column by eluting with 5% MeOH in DCM to afford methyl 2-((S)-3-cyclohexyl-2-(1H-indole-2-carboxamido)propanamido)-3-(2-oxo-1-azaspiro[4.5]decan-3-yl)propanoate (1). TLC system: 5% Methanol in DCM R_{f}: 0.65 LCMS (ESI): m/z 551.61 (M+H)⁺

### 2-((S)-3-cyclohexyl-2-(1H-indole-2-carboxamido)propanamido)-3-(2-oxo-1-azaspiro[4.5]decan-3-yl)propanoic acid (2)

To a stirred solution of methyl 2-((S)-3-cyclohexyl-2-(1H-indole-2-carboxamido)propanamido)-3-(2-oxo-1-azaspiro[4.5]decan-3-yl)propanoate (1) (1.3 g, 2.36 mmol) in THF (7 mL), water (3 mL) was added lithium hydroxide (170 mg, 7.09 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×20 mL), dried over sodium sulfate, concentrated under reduced pressure to afford 2-((S)-3-cyclohexyl-2-(1H-indole-2-carboxamido)propanamido)-3-(2-oxo-1-azaspiro[4.5]decan-3-yl)propanoic acid (2). TLC system: 10% MeOH in DCM Rf: 0.2 LCMS (ESI): m/z 537.64 [M+H]⁺

### 3-Chlorobenzyl ((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (4)

To a stirred solution of 2-((S)-2-((((3-chlorobenzyl)oxy)carbonyl)amino)-3-cyclohexylpropanamido)-3-(2-oxo-1-azaspiro[4.5]decan-3-yl)propanoic acid (2) (870 mg, 1.623 mmol) in THF (10 mL) added HATU (923 mg, 2.434 mmol), DIPEA (0.84 mL, 4.869 mmol) and 1-(cyanomethyl)tetrahydro-1H-thiophen-1-ium (3) (525 mg, 2.434 mmol) at 0° C. simultaneously and stirred at room temperature for 2 h. Reaction mixture was diluted with ice water (50 mL), extracted with EtOAc (2×20 mL), dried over sodium sulfate and evaporated under reduced pressure. The crude residue was purified by combi-flash NP, compound eluted at 5% methanol in dichloromethane to afford 3-chlorobenzyl ((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (4). TLC system: 10% Methanol in dichloromethane Rf: 0.4 LCMS (ESI): m/z 646.35 [M+H]⁺

### N-((2S)-1-((4-amino-3,4-dioxo-1-(2-oxo-1-azaspiro[4.5]decan-3-yl)butan-2-yl)amino)-3-cyclohexyl-1-oxopropan-2-yl)-1H-indole-2-carboxamide (Compound C119)

To a stirred solution N-((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)-1H-indole-2-carboxamide (4) (150 mg, 0.232 mmol) in methanol (3 mL) was added m-CPBA (100 mg, 0.580 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with MeOH in DCM (2×15 mL). Organic layer was washed with brine solution (30 mL), dried over Na₂SO₄ and concentrated to get crude compound. The crude compound was purified by prep-HPLC to afford N-((2S)-1-((4-amino-3,4-dioxo-1-(2-oxo-1-azaspiro[4.5]decan-3-yl)butan-2-yl)amino)-3-cyclohexyl-1-oxopropan-2-yl)-1H-indole-2-carboxamide (Compound C119). TLC system: 10% Methanol in dichloromethane Rf: 0.3 LCMS (ESI): m/z 564.2 [M+H]⁺

### Example 92: Synthesis of Compound C120

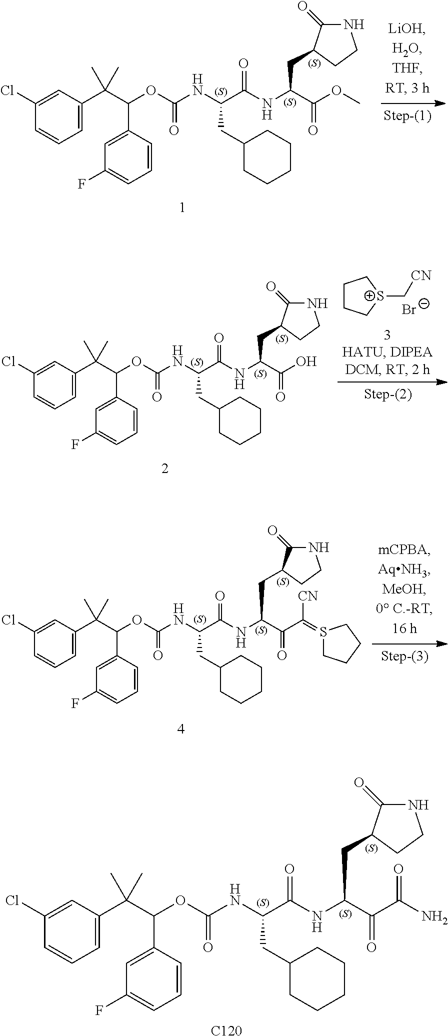

### (2S)-2-((2S)-2-(((2-(3-Chlorophenyl)-1-(3-fluorophenyl)-2-methylpropoxy) carbonyl) amino)-3-cyclohexylpropanamido)-3-((S)-2-oxopyrrolidin-3-yl) propanoic acid (2)

To a stirred solution of methyl (2S)-2-((2S)-2-(((2-(3-chlorophenyl)-1-(3-fluorophenyl)-2-methylpropoxy)carbonyl)amino)-3-cyclohexylpropanamido)-3-((S)-2-oxopyrrolidin-3-yl) propanoate (1) (1.1 g, 1.71 mmol) in THF (10 mL), water (4 mL) was added lithium hydroxide (214 mg, 5.13 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-((2S)-2-(((2-(3-chlorophenyl)-1-(3-fluorophenyl)-2-methylpropoxy)carbonyl)amino)-3-cyclohexyl propanamido)-3-((S)-2-oxopyrrolidin-3-yl) propanoic acid (2). TLC system: 10% MeOH in DCM Rf: 0.1 LCMS (ESI): m/z 630.59 [M+H]⁺

### 2-(3-Chlorophenyl)-1-(3-fluorophenyl)-2-methylpropyl ((S)-1-(((S)-4-cyano-3-oxo-1-((S)-2-oxopyrrolidin-3-yl)-4-(tetrahydro-1l4-thiophen-1-ylidene) butan-2-yl) amino)-3-cyclohexyl-1-oxopropan-2-yl) carbamate (4)

To a stirred solution of (2S)-2-((2S)-2-(((2-(3-chlorophenyl)-1-(3-fluorophenyl)-2-methyl propoxy)carbonyl)amino)-3-cyclohexylpropanamido)-3-((S)-2-oxopyrrolidin-3-yl)propanoic acid (2) (900 mg, 1.43 mmol) DCM (10 mL) added HATU (1 g, 2.86 mmol), DIPEA (8 mL, 4.29 mmol) and 1-(cyanomethyl)tetra hydro-1H-thiophen-1-iumbromide (3) (219 mg, 1.71 mmol) at 0° C. simultaneously and stirred at room temperature for 2 h. Reaction mixture was diluted with ice water (20 mL), extracted with dichloromethane (2×20 mL), dried over sodium sulfate and evaporated under reduced pressure. The crude residue was purified by combi-flash NP, compound eluted at 5% methanol in dichloromethane to afford 2-(3-chlorophenyl)-1-(3-fluorophenyl)-2-methylpropyl((S)-1-(((S)-4-cyano-3-oxo-1-((S)-2-oxopyrrolidin-3-yl)-4-(tetrahydro-1l4-thiophen-1-ylidene)butan-2-yl) amino)-3-cyclohexyl-1-oxopropan-2-yl) carbamate (4). TLC system: 10% Methanol in dichloromethane Rf: 0.4 LCMS (ESI): m/z 739.67 [M+H]⁺

### 2-(3-Chlorophenyl)-1-(3-fluorophenyl)-2-methylpropyl ((S)-1-(((S)-4-amino-3,4-dioxo-1-((S)-2-oxopyrrolidin-3-yl) butan-2-yl) amino)-3-cyclohexyl-1-oxopropan-2-yl) carbamate (Compound C120)

To a stirred solution of 2-(3-chlorophenyl)-1-(3-fluorophenyl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl) carbamate (4) (180 mg, 0.243 mmol) in methanol (2 mL) was added mCPBA (83 mg, 0.487 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (20 mL) and extracted with DCM (2×15 mL). Organic layer was washed with brine solution (20 mL), dried over Na₂SO₄ and concentrated to get crude compound. The crude compound was purified by prep HPLC to afford 2-(3-chlorophenyl)-1-(3-fluorophenyl)-2-methylpropyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C120). TLC system: 10% Methanol in dichloromethane Rf: 0.4 LCMS (ESI): m/z 657.1 [M+H]⁺

### Example 93: Synthesis of Compounds C128 and C122

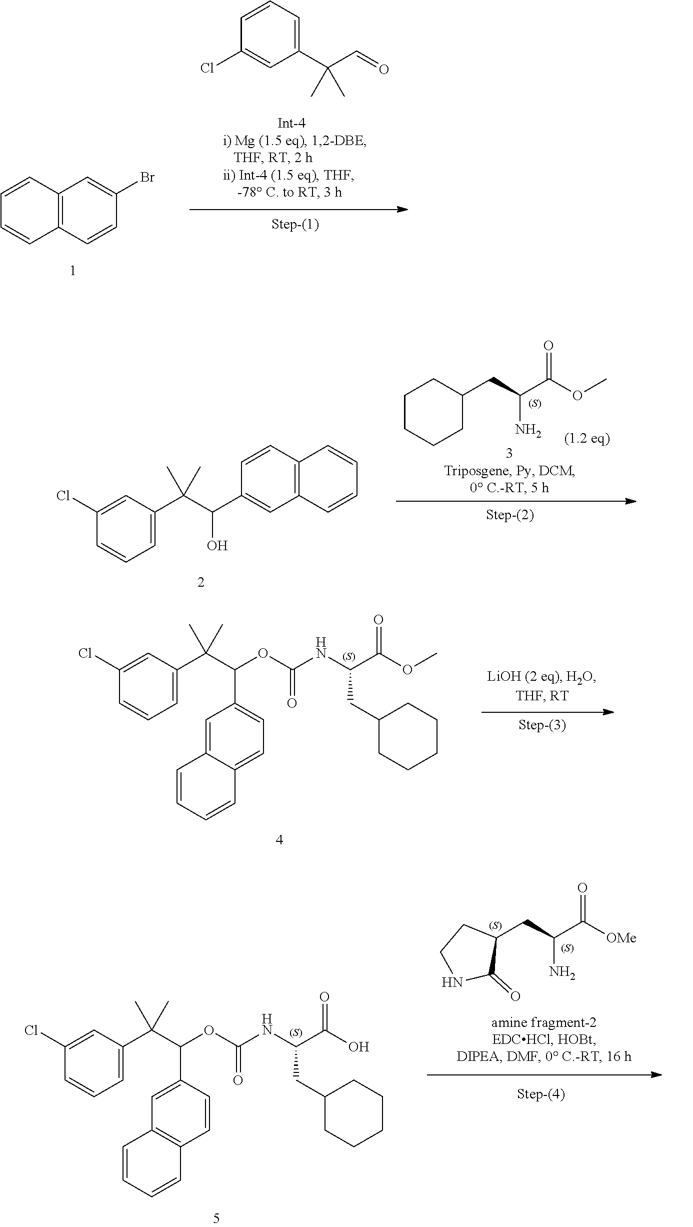

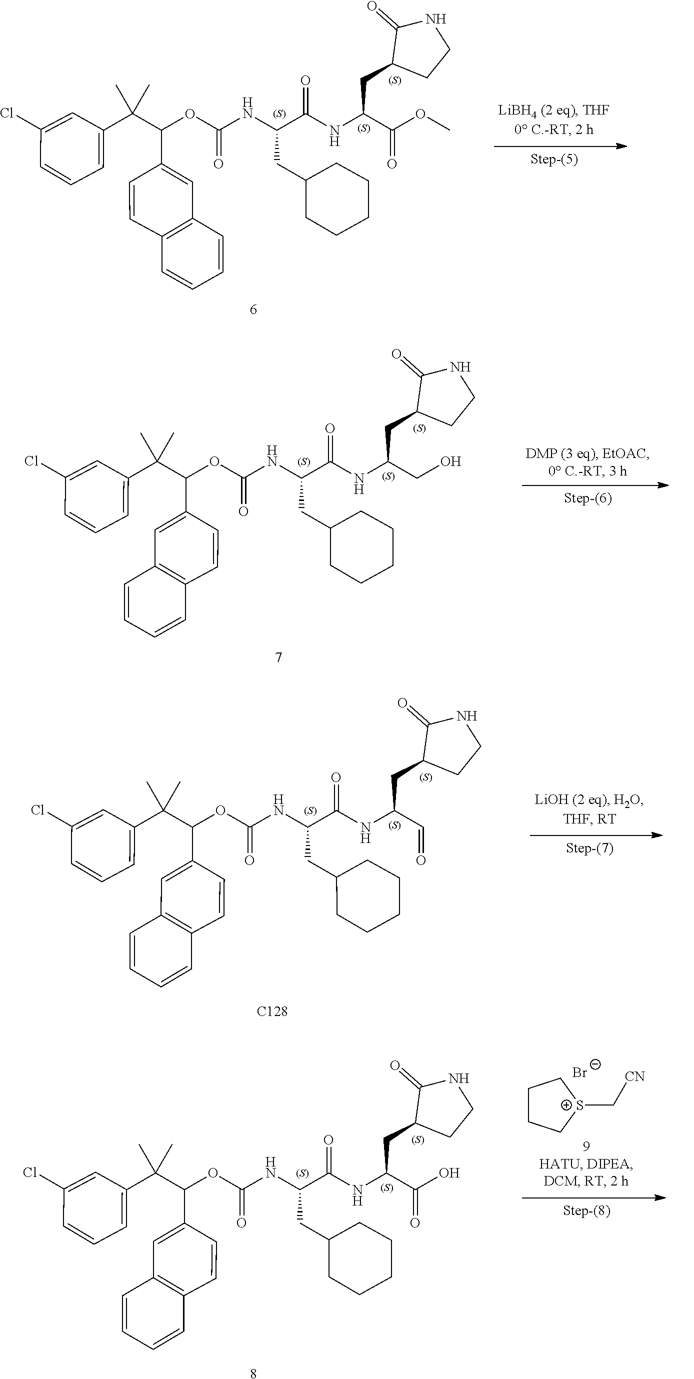

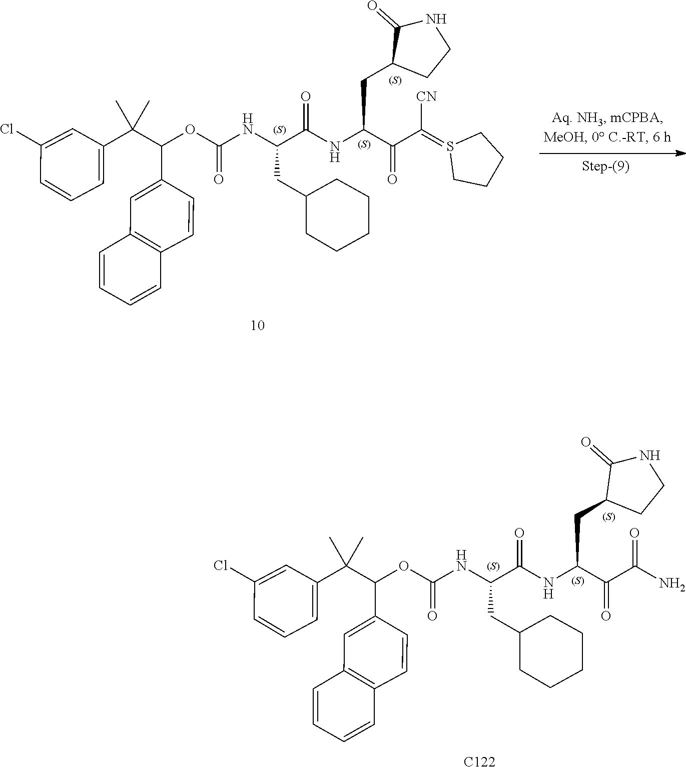

### 2-(3-Chlorophenyl)-2-methyl-1-(naphthalen-2-yl) propan-1-ol (2)

To a stirred solution of mg turnings (2.3 g, 96.61 mmol) and iodine (2 balls) in tetrahydrofuran (40 mL) was slowly added 2-bromonaphthalene (3) (8 g, 38.647 mmol) and 1,2 Dibromoethane at RT and stirred at 40° C. for 5 min reaction color was turned to color less and reaction mass stirred for 3 h. Another RBF (2-(3-chlorophenyl)-2-methylpropanal) (Int-4) (4 g, 21.97 mmol) in THF (40 mL) was cooled to −78° C. above prepared Grignard solution was slowly added at −78° C. stirred for 1 h. The progress of the reaction was monitored by TLC. Reaction mixture was quenched with saturated ammonium chloride solution and extracted with ethyl acetate (2×100 mL). Combined organic layer and washed with water (2×50 mL), dried over sodium sulfate, concentrated under reduced pressure to afford crude residue, crude residue was purified by normal phase chromatography 2-(3-chlorophenyl)-2-methyl-1-(naphthalen-2-yl)propan-1-ol (2). TLC system: 15% Ethyl acetate in hexane Rf: 0.4

### Methyl (2S)-2-(((2-(3-chlorophenyl)-2-methyl-1-(naphthalen-2-yl)propoxy)carbonyl)amino)-3-cyclohexylpropanoate (4)

To a stirred solution of chromatography 2-(3-chlorophenyl)-2-methyl-1-(naphthalen-2-yl) propan-1-ol (2) (3.5 g, 11.290 mmol), methyl (S)-2-amino-3-cyclohexylpropanoate hydrochloride (3) (2.5 g, 13.548 mmol) in DCM (40 mL) was added pyridine (3.5 mL, 1 vol) followed by triphosgene (1.67 g, 5.64 mmol) at 0° C. and stirred at room temperature for 3 h. The progress of the reaction was monitored by TLC and LCMS. Reaction mixture was diluted with DCM and washed with 1N HCl (50 mL) and washed with (2×50 mL) sat. NaHCO₃ solution, organic layer was dried over sodium sulfate, filtered and evaporated under reduced pressure. The crude residue was purified by combi-flash, compound eluted at 15% ethyl acetate in pet ether to afford methyl (2S)-2-(((2-(3-chlorophenyl)-2-methyl-1-(naphthalen-2-yl)propoxy)carbonyl)amino)-3-cyclohexyl propanoate (4). TLC system: 10% Ethyl acetate in hexane Rf: 0.3 LCMS (ESI): m/z 544.52 [M+Na]⁺

### (2S)-2-(((2-(3-chlorophenyl)-2-methyl-1-(naphthalen-2-yl)propoxy)carbonyl)amino)-3-cyclohexylpropanoic acid (5)

To a stirred solution of methyl (2S)-2-(((2-(3-chlorophenyl)-2-methyl-1-(naphthalen-2-yl)propoxy)carbonyl)amino)-3-cyclohexyl propanoate (4) (2.8 g, 5.374 mmol) in THF (38 mL), water (10 mL) was added lithium hydroxide (661 mg, 16.37 mmol) at 0° C. and stirred at room temperature for 5 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford (2S)-2-(((2-(3-chlorophenyl)-2-methyl-1-(naphthalen-2-yl)propoxy)carbonyl)amino)-3-cyclohexylpropanoic acid (5). TLC system: 50% Ethyl acetate in hexane Rf: 0.2 LCMS (ESI): m/z 530.33 [M+Na]⁺

### Methyl (2S)-2-((2S)-2-(((2-(3-chlorophenyl)-2-methyl-1-(naphthalen-2-yl)propoxy)carbonyl)amino)-3-cyclohexylpropanamido)-3-((S)-2-oxopyrrolidin-3-yl)propanoate (6)

To a stirred solution of (2S)-2-(((2-(3-chlorophenyl)-2-methyl-1-(naphthalen-2-yl) propoxy)carbonyl)amino)-3-cyclohexylpropanoic acid (5) (1.9 g, 3.74 mmol) DMF (20 mL) added EDC.HCl (1.07 g, 5.621 mmol), HOBt (758 mg, 5.621 mmol), DIPEA (2 mL, 11.242 mmol) and methyl (S)-2-amino-3-((S)-2-oxopyrrolidin-3-yl)propanoate hydrochloride (amine fragment-2) (0.836 g, 4.497 mmol) at 0° C. simultaneously and stirred at room temperature for 16 h. Reaction mixture was quenched with ice water (80 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% methanol in dichloromethane to afford Methyl (2S)-2-((2S)-2-(((2-(3-chlorophenyl)-2-methyl-1-(naphthalen-2-yl)propoxy)carbonyl)amino)-3-cyclohexyl propanamido)-3-((S)-2-oxopyrrolidin-3-yl)propanoate (6). TLC system: 80% Ethyl acetate in hexane Rf: 0.1 LCMS (ESI): m/z 676.33 [M+H]⁺

### 2-(3-Chlorophenyl)-2-methyl-1-(naphthalen-2-yl)propyl ((S)-3-cyclohexyl-1-(((S)-1-hydroxy-3-((S)-2-oxopyrrolidin-3-yl)propan-2-yl)amino)-1-oxopropan-2-yl)carbamate (7)

To a stirred solution of methyl (2S)-2-((2S)-2-(((2-(3-chlorophenyl)-2-methyl-1-(naphthalen-2-yl)propoxy)carbonyl)amino)-3-cyclohexyl propanamido)-3-((S)-2-oxopyrrolidin-3-yl)propanoate (6) (400 mg, 0.592 mmol) in THF (10 mL) was added 2M LiBH₄ in THF (0.6 mL, 1.18 mmol) at 0° C. and the reaction mixture stirred for 2 h at 0° C. The progress of the reaction was monitored by TLC and LCMS. Reaction mixture was quenched with sat. Ammonium chloride solution (20 mL) and extracted with DCM (2×20 mL). Organic layer was washed with brine solution (30 mL), dried over Na₂SO₄ and concentrated to afford 2-(3-chlorophenyl)-2-methyl-1-(naphthalen-2-yl)propyl((S)-3-cyclohexyl-1-(((S)-1-hydroxy-3-((S)-2-oxopyrrolidin-3-yl)propan-2-yl)amino)-1-oxopropan-2-yl)carbamate (7). TLC system: 100% Ethyl acetate Rf: 0.2 LCMS (ESI): m/z 648.73 [M+H]⁺

### 2-(3-Chlorophenyl)-2-methyl-1-(naphthalen-2-yl)propyl ((S)-3-cyclohexyl-1-oxo-1-(((S)-1-oxo-3-((S)-2-oxopyrrolidin-3-yl)propan-2-yl)amino)propan-2-yl)carbamate (Compound C128)

To a stirred solution of 2-(3-chlorophenyl)-2-methyl-1-(naphthalen-2-yl)propyl((S)-3-cyclohexyl-1-(((S)-1-hydroxy-3-((S)-2-oxopyrrolidin-3-yl)propan-2-yl)amino)-1-oxopropan-2-yl) carbamate (7) (300 mg, 0.46 mmol) was dissolved in Ethyl acetate (5 mL) was added Dess-Martin periodinane (589 mg, 1.39 mmol) at 0° C. and stirred at RT for 3 h. Reaction mixture was Filter through celite pad washed with ethyl acetate (20 mL) filtrate was washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford 2-(3-chlorophenyl)-2-methyl-1-(naphthalen-2-yl)propyl ((S)-3-cyclohexyl-1-oxo-1-(((S)-1-oxo-3-((S)-2-oxopyrrolidin-3-yl)propan-2-yl)amino)propan-2-yl)carbamate (Compound C128). TLC system: 10% Methanol in dichloromethane Rf: 0.4 LCMS (ESI): m/z 646.2 (M+H)⁺

### (2S)-2-((2S)-2-(((2-(3-Chlorophenyl)-2-methyl-1-(naphthalen-2-yl)propoxy)carbonyl)amino)-3-cyclohexylpropanamido)-3-((S)-2-oxopyrrolidin-3-yl)propanoic acid (8)

To a stirred solution of methyl (2S)-2-((2S)-2-(((2-(3-chlorophenyl)-2-methyl-1-(naphthalen-2-yl) propoxy)carbonyl)amino)-3-cyclohexylpropanamido)-3-((S)-2-oxopyrrolidin-3-yl)propanoate (6) (2 g, 2.962 mmol) in THF (15 mL), water (10 mL) was added lithium hydroxide (0.364 g, 8.88 mmol) at 0° C. and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×10 mL), dried over sodium sulfate, concentrated under reduced pressure to afford (2S)-2-((2S)-2-(((2-(3-chlorophenyl)-2-methyl-1-(naphthalen-2-yl)propoxy)carbonyl)amino)-3-cyclohexylpropanamido)-3-((S)-2-oxopyrrolidin-3-yl)propanoic acid (8) TLC system: 100% Ethyl acetate Rf: 0.2 LCMS (ESI): m/z 684.49 [M+Na]⁺

### 2-(3-Chlorophenyl)-2-methyl-1-(naphthalen-2-yl)propyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (10)

To a stirred solution of (2S)-2-((2S)-2-(((2-(3-chlorophenyl)-2-methyl-1-(naphthalen-2-yl)propoxy)carbonyl)amino)-3-cyclohexylpropanamido)-3-((S)-2-oxopyrrolidin-3-yl)propanoic acid (8) (2×300 mg, 0.453 mmol) DCM (10 mL) added HATU (258 mg, 0.680 mmol), DIPEA (0.4 mL, 2.178 mmol) and 1-(cyanomethyl)tetrahydro-1H-thiophen-1-iumbromide (9) (140 mg, 0.680 mmol) at 0° C. simultaneously and stirred at room temperature for 2 h. Reaction mixture was diluted with ice water (25 mL), extracted with 5% methanol in dichloromethane (2×15 mL), dried over sodium sulfate and evaporated under reduced pressure. The crude residue was purified by combi-flash NP, compound eluted at 5% methanol in dichloromethane to afford 2-(3-chlorophenyl)-2-methyl-1-(naphthalen-2-yl)propyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (10). TLC system: 10% Methanol in dichloromethane Rf: 0.3 LCMS (ESI): m/z 771.71 [M+H]⁺

### 2-(3-Chlorophenyl)-2-methyl-1-(naphthalen-2-yl)propyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C122)

To a stirred solution of 2-(3-chlorophenyl)-2-methyl-1-(naphthalen-2-yl)propyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl) carbamate (10) (500 mg, 0.649 mmol) in methanol (5 mL) was added m-CPBA (279 mg, 1.623 mmol) at 0° C. and the reaction mixture stirred for 2 h at 0° C. and added aq ammonia (2.5 mL) and stirred at RT for 6 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford 2-(3-chlorophenyl)-2-methyl-1-(naphthalen-2-yl)propyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C122). TLC system: 10% Methanol in DCM and 100% ethyl acetate Rf: 0.3 LCMS (ESI): m/z 689.2 [M+H]⁺

### Example 94: Synthesis of Compound C123

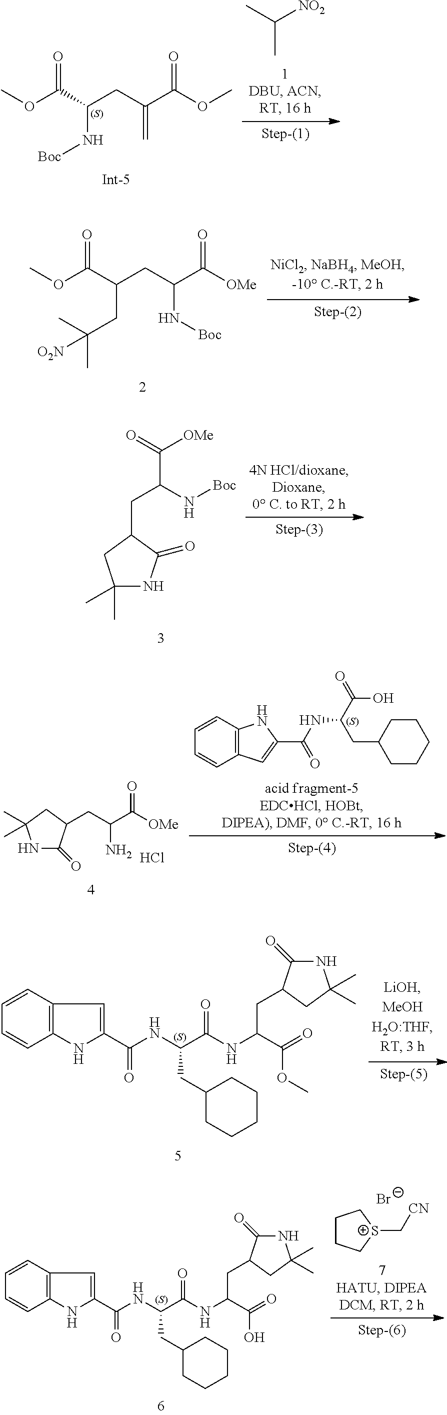

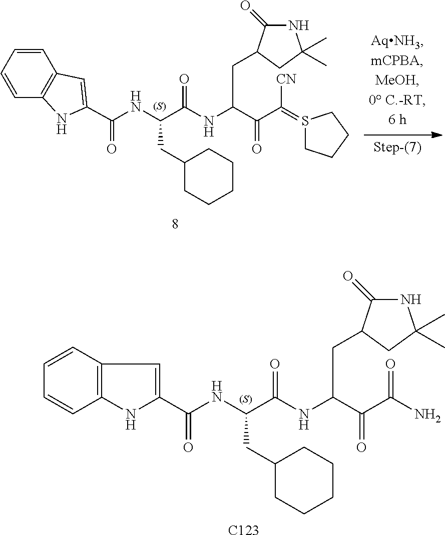

### Dimethyl 2-((tert-butoxycarbonyl) amino)-4-(2-methyl-2-nitropropyl) pentanedioate (2)

To a stirred solution of 2-nitropropane (1) (2 g, 22.47 mmol) in ACN (60 mL), dimethyl (S)-2-((tert-butoxy carbonyl) amino)-4-methylenepentanedioate (Int-5) (7.4 g, 24.71 mmol) was and DBU (6 mL, 44.94 mmol) added and stirred at room temperature for 16 h. The progress of the reaction was monitored by TLC and LCMS. Reaction mixture completely distilled under reduced pressure afford crude product: this crude was diluted with water and extracted with ethyl acetate (2×50 mL), dried over sodium sulfate and concentrated under reduced pressure. The crude residue was purified by combi-flash NP, compound eluted at 30% ethyl acetate in hexane to dimethyl 2-((tert-butoxycarbonyl) amino)-4-(2-methyl-2-nitropropyl) pentanedioate (2). TLC system: 30% Ethyl acetate in hexane Rf: 0.2 LCMS (ESI): m/z 377.14 [M+H]⁺

### Methyl 2-((tert-butoxycarbonyl) amino)-3-(5,5-dimethyl-2-oxopyrrolidin-3-yl) propanoate (3)

To a stirred solution of dimethyl 2-((tert-butoxy carbonyl) amino)-4-(2-methyl-2-nitropropyl) pentanedioate (2) (3 g, 7.97 mmol) in MeOH (30 mL) added NiCl₂ (1 g, 7.97 mmol), and stirred at −10° C. for 10 minutes then added NaBH₄ (1.5 g, 39.89 mmol) at same temperature for 2 h. The progress of the reaction was monitored by TLC and LCMS. Reaction mixture was quenched with sat. NH₄Cl (100 mL), filtered through celite pad and the filtrate was extracted with DCM (2×40 mL), dried over sodium sulfate and evaporated under reduced pressure afford to methyl 2-((tert-butoxycarbonyl) amino)-3-(5,5-dimethyl-2-oxopyrrolidin-3-yl) propanoate (3). TLC system: 100% Ethyl acetate Rf: 0.4 LCMS (ESI): m/z 337.12 [M+Na]⁺

### Methyl 2-amino-3-(5,5-dimethyl-2-oxopyrrolidin-3-yl) propanoate hydrochloride (4)

To a stirred solution methyl 2-((tert-butoxycarbonyl) amino)-3-(5,5-dimethyl-2-oxopyrrolidin-3-yl) propanoate (3) (2.3 g, 7.32 mmol) in 1,4 dioxane (20 mL) at 0° C. was added drop wise 4N HCl in dioxane (10 ml) and the reaction mixture was stirred at RT for 2 h. The progress of the reaction was monitored by TLC. After consumption of starting material, the reaction mixture was evaporated under reduced pressure to obtained crude compound, the resulting crude triturated with diethyl ether to afford methyl 2-amino-3-(5,5-dimethyl-2-oxopyrrolidin-3-yl)propanoate hydrochloride (4). TLC system: 20% Methanol in DCM Rf: 0.1 LCMS (ESI): m/z 228.18 [M+H]⁺

### Methyl 2-((S)-3-cyclohexyl-2-(1H-indole-2-carboxamido) propanamido)-3-(5,5-dimethyl-2-oxopyrrolidin-3-yl) propanoate (5)

To a stirred solution of (S)-3-cyclohexyl-2-(1H-indole-2-carboxamido)propanoic acid (acid fragment-5) (1.5 g, 4.77 mmol) DMF (15 mL) added EDC.HCl (1.3 g, 7.150 mmol), HOBt (965 mg, 7.150 mmol), DIPEA (2 mL, 14.31 mmol) and methyl 2-amino-3-(5,5-dimethyl-2-oxopyrrolidin-3-yl)propanoate hydrochloride (4) (1.4 g, 5.721 mmol) at 0° C. simultaneously and stirred at room temperature for 16 h. Reaction mixture was diluted with ice water (80 mL), extracted with ethyl acetate (2×60 mL), dried over sodium sulfate and evaporated under reduced pressure. The crude residue was purified by combi-flash NP, compound eluted at 3% methanol in dichloromethane to methyl 2-((S)-3-cyclohexyl-2-(1H-indole-2-carboxamido) propanamido)-3-(5,5-dimethyl-2-oxopyrrolidin-3-yl) propanoate (5). TLC system: 10% Methanol in dichloromethane Rf: 0.4 LCMS (ESI): m/z 511.64 [M+H]⁺

### 2-((S)-3-Cyclohexyl-2-(1H-indole-2-carboxamido) propanamido)-3-(5,5-dimethyl-2-oxopyrrolidin-3-yl) propanoic acid (6)

To a stirred solution of methyl 2-((S)-3-cyclohexyl-2-(1H-indole-2-carboxamido)propanamido)-3-(5,5-dimethyl-2-oxopyrrolidin-3-yl)propanoate (5) (1 g, 1.96 mmol) in THF (15 mL), water (5 mL) was added lithium hydroxide (241 mg, 5.88 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford 2-((S)-3-cyclohexyl-2-(1H-indole-2-carboxamido)propanamido)-3-(5,5-dimethyl-2-oxopyrrolidin-3-yl)propanoic acid (6). TLC system: 100% EtOAc Rf: 0.1 LCMS (ESI): m/z 497.75 [M+H]⁺

### N-((2S)-1-((4-Cyano-1-(5,5-dimethyl-2-oxopyrrolidin-3-yl)-3-oxo-4-(tetrahydro-1l4-thiophen-1-ylidene)butan-2-yl)amino)-3-cyclohexyl-1-oxopropan-2-yl)-1H-indole-2-carboxamide (8)

To a stirred solution of 2-((S)-3-cyclohexyl-2-(1H-indole-2-carboxamido)propanamido)-3-(5,5-dimethyl-2-oxopyrrolidin-3-yl)propanoic acid (6) (500 mg, 1.00 mmol) DCM (5 mL) added HATU (574 mg, 1.511 mmol), DIPEA (0.6 mL, 4.53 mmol) and 1-(cyanomethyl) tetrahydro-1H-thiophen-1-iumbromide (7) (311 mg, 1.511 mmol) at 0° C. simultaneously and stirred at room temperature for 2 h. Reaction mixture was diluted with ice water (30 mL), extracted with dichloromethane (2×30 mL), dried over sodium sulfate and evaporated under reduced pressure. The crude residue was purified by combi-flash NP, compound eluted at 5% methanol in dichloromethane to afford N-((2S)-1-((4-cyano-1-(5,5-dimethyl-2-oxopyrrolidin-3-yl)-3-oxo-4-(tetrahydro-1l4-thiophen-1-ylidene)butan-2-yl)amino)-3-cyclohexyl-1-oxopropan-2-yl)-1H-indole-2-carboxamide (8). TLC system: 10% Methanol in dichloromethane Rf: 0.5 LCMS (ESI): m/z 606.69 [M+H]⁺

### N-((2S)-1-((4-amino-1-(5,5-dimethyl-2-oxopyrrolidin-3-yl)-3,4-dioxobutan-2-yl) amino)-3-cyclohexyl-1-oxopropan-2-yl)-1H-indole-2-carboxamide (Compound C123)

To a stirred solution of N-((2S)-1-((4-cyano-1-(5,5-dimethyl-2-oxopyrrolidin-3-yl)-3-oxo-4-(tetrahydro-1l4-thiophen-1-ylidene)butan-2-yl)amino)-3-cyclohexyl-1-oxopropan-2-yl)-1H-indole-2-carboxamide (8) (200 mg, 0.33 mmol) in methanol (3 mL) was added mCPBA (113 mg, 0.66 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×20 mL). Organic layer was washed with brine solution (30 mL), dried over Na₂SO₄ and concentrated to get crude compound. The crude compound was purified by prep HPLC to afford N4(2S)-1-((4-amino-1-(5,5-dimethyl-2-oxopyrrolidin-3-yl)-3,4-dioxobutan-2-yl)amino)-3-cyclohexyl-1-oxopropan-2-yl)-1H-indole-2-carboxamide (Compound C123). TLC system: 10% Methanol in dichloromethane Rf: 0.3 LCMS (ESI): m/z 524.2 (M+H)⁺

### Example 95: Synthesis of Compounds C124 and C136

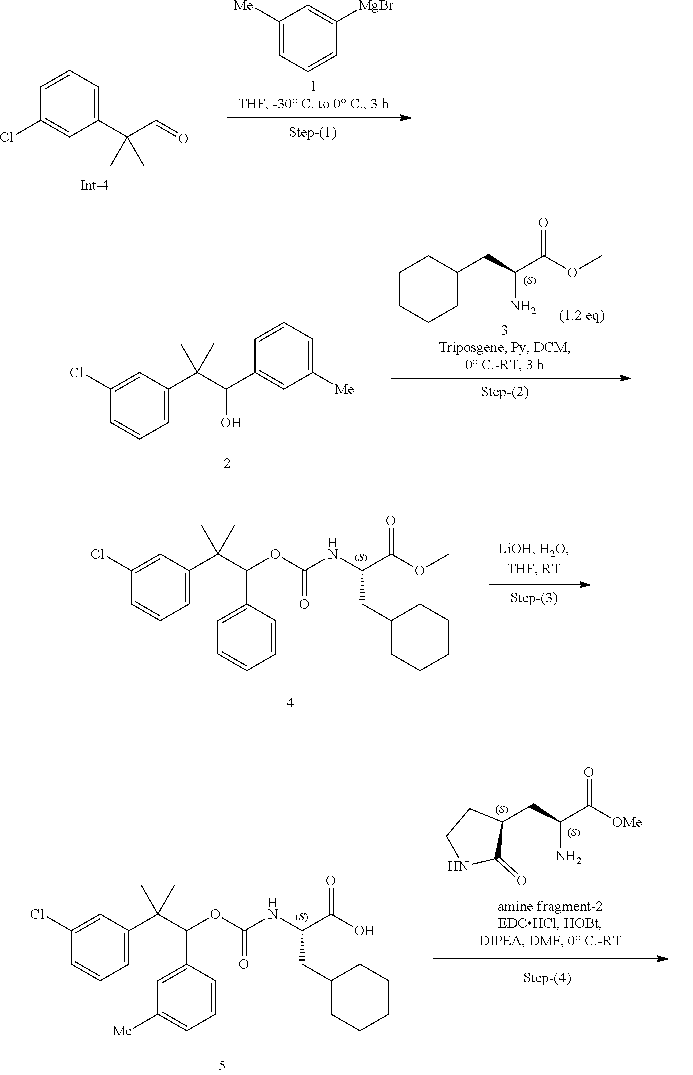

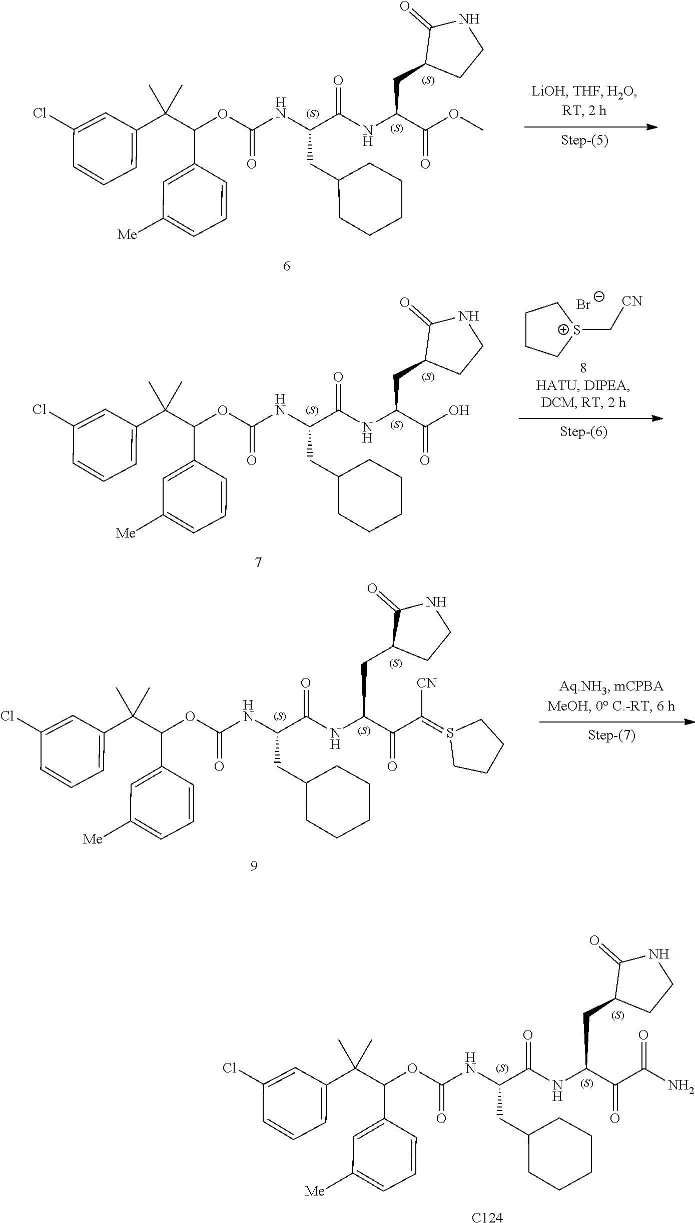

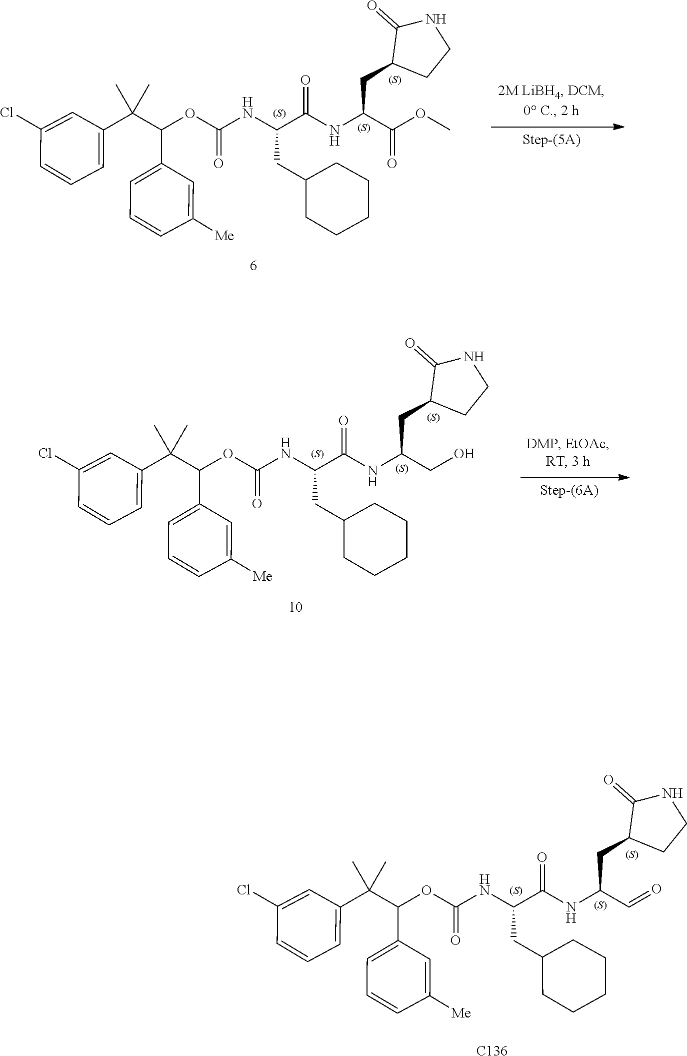

### 2-(3-chlorophenyl)-2-methyl-1-(m-tolyl) propan-1-ol (2)

To a stirred solution of 2-(3-chlorophenyl)-2-methylpropanal (Int-4) (7 g, 38.461 mmol) in THF (60 mL) was added 3-methyl magnesium bromide (115 mL, 115.384 mmol) at −30° C. and the reaction mixture was stirred at 0° C. for 3 h. The progress of the reaction was monitored by TLC. The reaction mixture was quenched with saturated NH₄Cl (50 mL) and filtered through celite pad and washed with ethyl acetate (100 mL), dried over sodium sulfate and evaporated under reduced pressure. The crude residue was purified by silica gel column by eluting with 10% ethyl acetate in hexane to afford 2-(3-chlorophenyl)-2-methyl-1-(m-tolyl)propan-1-ol (2). TLC system: 10% Ethyl acetate in Hexane Rf: 0.3 LCMS (ESI): m/z=258.81 [M−OH]

### Methyl (2S)-2-(((2-(3-chlorophenyl)-2-methyl-1-(m-tolyl) propoxy) carbonyl) amino)-3-cyclohexylpropanoate (4)

To a stirred solution of 2-(3-chlorophenyl)-2-methyl-1-(m-tolyl)propan-1-ol (2) (6 g, mmol) in DCM (60 mL) was added pyridine (6 mL) methyl L-leucinate hydrochloride (3) (3.8 g, 26.277 mmol) followed by triphosgene (3.24 g, 10.948 mmol) at 0° C. slowly and stirred at RT for 16 h. The progress of the reaction was monitored by TLC. Reaction mixture was quenched with 1N aq. HCl (100 mL) then extracted with DCM (2×50 mL), washed with (2×50 mL) NaHCO₃ solution the organic layer was dried over sodium sulfate and evaporated under reduced pressure. The crude residue was purified by silica gel column by eluting with 15% ethyl acetate in hexane to afford methyl (2S)-2-(((2-(3-chlorophenyl)-2-methyl-1-(m-tolyl)propoxy)carbonyl)amino)-3-cyclohexylpropanoate (4). TLC system: 10% Ethyl acetate in hexane Rf: 0.3 LCMS (ESI): m/z=508.53 [M+Na]+

### ((1-(3-(Benzyloxy)benzyl)cyclopropoxy)carbonyl)-L-leucine (5)

To a stirred solution of methyl (2S)-2-(((2-(3-chlorophenyl)-2-methyl-1-(m-tolyl) propoxy) carbonyl) amino)-3-cyclohexylpropanoate (4) (4.8 g, 9.896 mmol) in THF (20 mL), water (5 mL) was added LiOH.H₂O (712 mg, 29.690 mmol) at 0° C. Reaction mixture was stirred at RT for 3 h. The progress of the reaction was monitored by TLC. After consumption of starting material, the reaction mixture was concentrated and acidified with 1N HCl, extracted with ethyl acetate (2×50 mL), dried over sodium sulfate and evaporated under reduced pressure to afford ((1-(3-(benzyloxy)benzyl)cyclopropoxy)carbonyl)-L-leucine (5). TLC system: 10% Methanol in dichloromethane Rf: 0.2 LCMS (ESI): m/z=470.18 [M−H]

### Methyl (2S)-2-((2S)-2-(((2-(3-chlorophenyl)-2-methyl-1-(m-tolyl) propoxy) carbonyl) amino)-3-cyclohexylpropanamido)-3-((S)-2-oxopyrrolidin-3-yl) propanoate (6)

To a stirred solution of ((1-(3-(benzyloxy)benzyl)cyclopropoxy)carbonyl)-L-leucine (5) (2 g, 4.246 mmol) in DMF (20 mL) was added EDC.HCl (1.2 g, 6.309 mmol), HOBt (0.859 g, 6.369 mmol), DIPEA (2.3 mL, 12.738 mmol) and methyl (S)-2-amino-3-((S)-2-oxopyrrolidin-3-yl)propanoate (amine fragment-2) (0.947 g, 5.095 mmol) at 0° C. simultaneously and stirred at room temperature for 16 h. Reaction mixture was diluted with ice water (100 mL), extracted with ethyl acetate (2×50 mL), the organic layer was dried over sodium sulfate and evaporated under reduced pressure to afford crude compound. The crude residue was purified by silica gel column by eluting with 50% ethyl acetate and pet-ether to afford methyl (2S)-2-((2S)-2-(((2-(3-chlorophenyl)-2-methyl-1-(m-tolyl)propoxy)carbonyl)amino)-3-cyclohexylpropanamido)-3-((S)-2-oxopyrrolidin-3-yl)propanoate (6). TLC system: 10% Methanol in dichloromethane Rf: 0.3 LCMS (ESI): m/z=640.35 [M+H]⁺

### (2S)-2-((2S)-2-(((2-(3-chlorophenyl)-2-methyl-1-(m-tolyl) propoxy) carbonyl) amino)-3-cyclohexylpropanamido)-3-((S)-2-oxopyrrolidin-3-yl) propanoic acid (7)

To a stirred solution of methyl (2S)-2-((2S)-2-(((2-(3-chlorophenyl)-2-methyl-1-(m-tolyl)propoxy)carbonyl)amino)-3-cyclohexylpropanamido)-3-((S)-2-oxopyrrolidin-3-yl) propanoate (6) (550 mg, 0.86 mmol) in THF (10 mL), water (5 mL) was added lithium hydroxide (61 mg, 2.582 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-((2S)-2-(((2-(3-chlorophenyl)-2-methyl-1-(m-tolyl)propoxy)carbonyl)amino)-3-cyclohexyl propan amido)-3-((S)-2-oxopyrrolidin-3-yl) propanoic acid (7). TLC system: 15% Methanol in dichloromethane Rf: 0.2 LCMS (ESI): m/z 626.31 [M+H]⁺

### 2-(3-Chlorophenyl)-2-methyl-1-(m-tolyl) propyl ((S)-1-(((S)-4-cyano-3-oxo-1-((S)-2-oxopyrrolidin-3-yl)-4-(tetrahydro-1l4-thiophen-1-ylidene) butan-2-yl) amino)-3-cyclohexyl-1-oxopropan-2-yl) carbamate (9)

To a stirred solution of (2S)-2-((2S)-2-(((2-(3-chlorophenyl)-2-methyl-1-(m-tolyl) propoxy) carbonyl) amino)-3-cyclo hexylpropanamido)-3-((S)-2-oxopyrrolidin-3-yl) propanoic acid (7) (300 mg, 0.480 mmol) DCM (10 mL) added HATU (273 mg, 0.720 mmol), DIPEA (0.26 mL, 1.44 mmol) and 1-(cyan methyl)tetra hydro-1H-thiophen-1-iumbromide (8) (92 mg, 0.720 mmol) at 0° C. simultaneously and stirred at room temperature for 2 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 2-(3-chlorophenyl)-2-methyl-1-(m-tolyl)propyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (9). TLC system: 10% Methanol in dichloromethane Rf: 0.4 LCMS (ESI): m/z 735.41 [M+H]⁺

### 2-(3-Chlorophenyl)-2-methyl-1-(m-tolyl) propyl ((S)-1-(((S)-4-amino-dioxo-1-((S)-2-oxopyrrolidin-3-yl) butan-2-yl) amino)-3-cyclohexyl-1-oxopropan-2-yl) carbamate (Compound C124)

To a stirred solution of 2-(3-chlorophenyl)-2-methyl-1-(m-tolyl)propyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (9) (200 mg, 0.272 mmol) in methanol (3 mL) was added mCPBA (93 mg, 0.544 mmol) at 0° C. and the reaction mixture stirred for 2 h at 0° C. and added aq ammonia (2 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep-HPLC to afford 2-(3-chlorophenyl)-2-methyl-1-(m-tolyl)propyl ((S)-1-(((S)-4-amino-3,4-dioxo-1-((S)-2-oxopyrrolidin-3-yl)butan-2-yl) amino)-3-cyclohexyl-1-oxopropan-2-yl) carbamate (Compound C124). TLC system: 10% Methanol in dichloromethane Rf: 0.3 LCMS (ESI): m/z 653.2 [M+H]⁺

### 2-(3-Chlorophenyl)-2-methyl-1-(m-tolyl) propyl ((S)-3-cyclohexyl-1-(((S)-1-hydroxy-3-((S)-2-oxopyrrolidin-3-yl) propan-2-yl) amino)-1-oxopropan-2-yl)carbamate (10)

To a stirred solution of methyl (2S)-2-((2S)-2-(((2-(3-chlorophenyl)-2-methyl-1-(m-tolyl)propoxy)carbonyl)amino)-3-cyclohexylpropanamido)-3-((S)-2-oxopyrrolidin-3-yl) propanoate (6) (1 g, 1.564 mmol) in DCM (10 mL) was added 2M LiBH₄ in THF (1.9 mL, 3.912 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to get compound to afford 2-(3-chlorophenyl)-2-methyl-1-(m-tolyl)propyl((S)-3-cyclohexyl-1-(((S)-1-hydroxy-3-((S)-2-oxopyrrolidin-3-yl) propan-2-yl)amino)-1-oxopropan-2-yl) carbamate (10). TLC system: 10% Methanol in DCM Rf: 0.4 LCMS (ESI): m/z 612.7 (M+H)⁺

### 2-(3-Chlorophenyl)-2-methyl-1-(m-tolyl) propyl ((S)-3-cyclohexyl-1-oxo-1-(((S)-1-oxo-3-((S)-2-oxopyrrolidin-3-yl) propan-2-yl) amino) propan-2-yl) carbamate (Compound C136)

To a stirred solution of 2-(3-chlorophenyl)-2-methyl-1-(m-tolyl)propyl ((S)-3-cyclohexyl-1-(((S)-1-hydroxy-3-((S)-2-oxopyrrolidin-3-yl) propan-2-yl)amino)-1-oxopropan-2-yl)carbamate (5A) (300 mg, 0.490 mmol) was dissolved in ethyl acetate (10 mL) was added Dess-Martin periodinane (416 mg, 0.981 mmol) at 0° C. and stirred at RT for 3 h. Reaction mixture was diluted with ethyl acetate (20 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by combi-flash chromatography by eluting 3% methanol in dichloromethane to afford 2-(3-chlorophenyl)-2-methyl-1-(m-tolyl)propyl ((S)-3-cyclohexyl-1-oxo-1-(((S)-1-oxo-3-((S)-2-oxopyrrolidin-3-yl)propan-2-yl)amino)propan-2-yl)carbamate (Compound C136). TLC system: 10% Methanol in DCM Rf: 0.4 LCMS (ESI): m/z 610.2 (M+H)⁺

### Example 96: Synthesis of Compounds C141 and C126

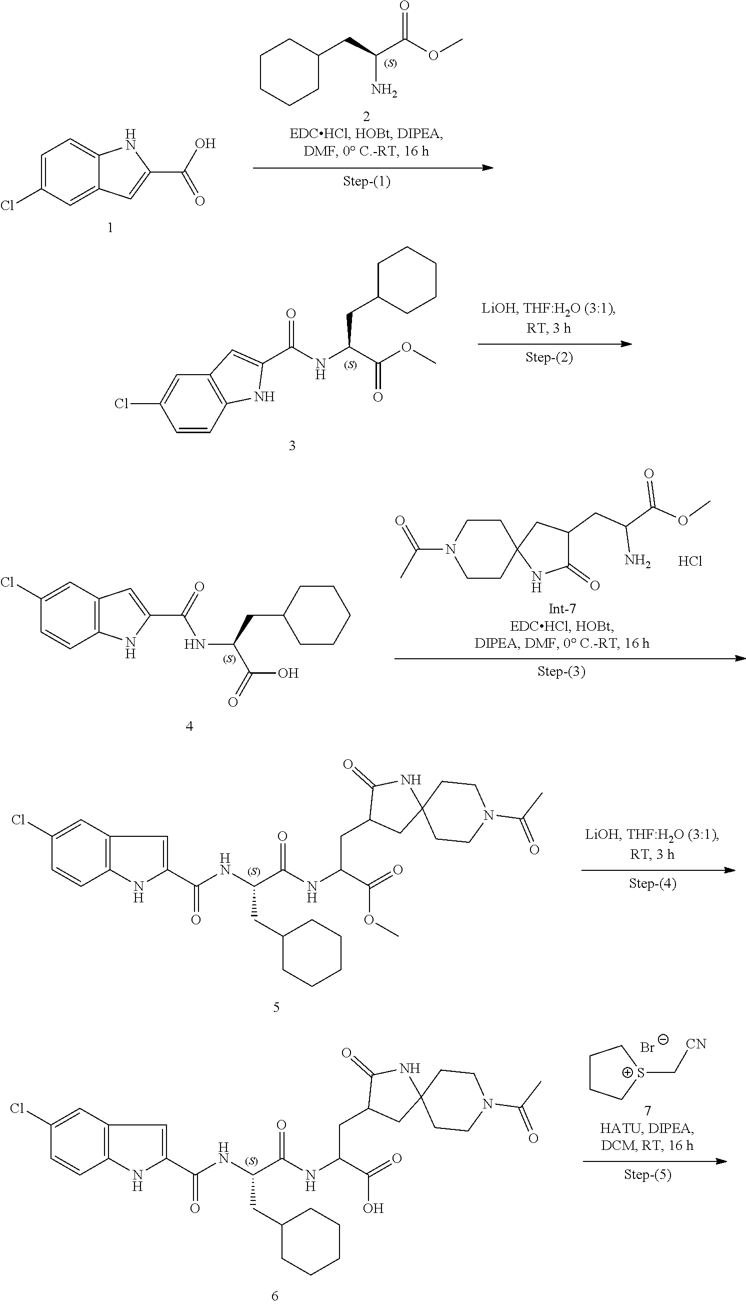

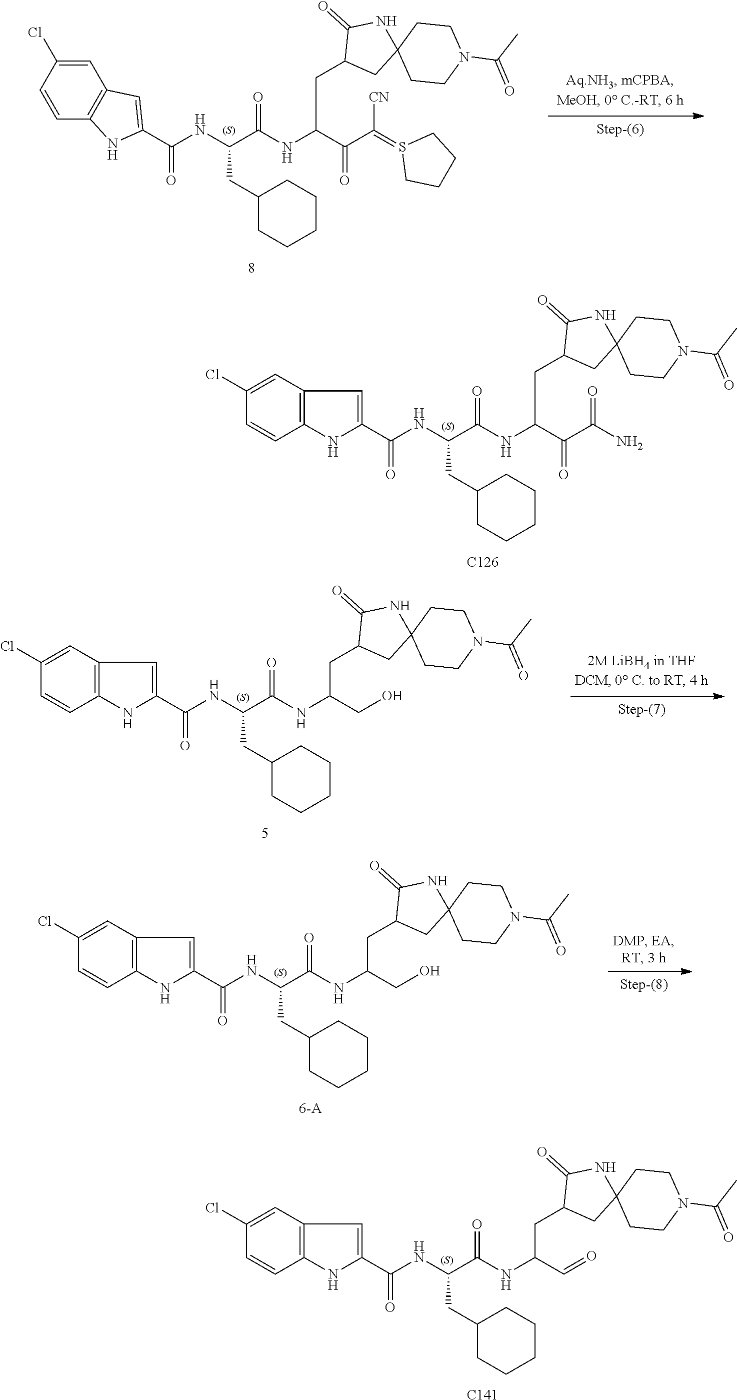

### Methyl (S)-2-(5-chloro-1H-indole-2-carboxamido)-3-cyclohexylpropanoate (3)

To a stirred solution of 5-chloro-1H-indole-2-carboxylic acid (1) (3 g, 15.3 mmol) DMF (30 mL) added EDC.HCl (4.3 g, 22.95 mmol), HOBt (3.0 g, 22.95 mmol), DIPEA (8.0 mL, 45.9 mmol) and methyl (S)-2-amino-3-cyclohexylpropanoate (2) (2.8 g, 15.3 mmol) at 0° C. simultaneously and stirred at room temperature for 16 h. Reaction mixture was diluted with ice water (80 mL), extracted with ethyl acetate (2×60 mL), dried over sodium sulfate and evaporated under reduced pressure. The crude residue was purified by combi-flash NP, compound eluted at 20% Ethyl acetate in hexane to methyl (S)-2-(5-chloro-1H-indole-2-carboxamido)-3-cyclohexylpropanoate (3). TLC system: 20% Ethyl acetate in hexane Rf: 0.2 LCMS (ESI): m/z 363.08 [M+H]⁺

### (S)-2-(5-chloro-1H-indole-2-carboxamido)-3-cyclohexylpropanoic acid (4)

To a stirred solution of methyl (S)-2-(5-chloro-1H-indole-2-carboxamido)-3-cyclohexylpropanoate (3) (3.5 g, 9.6 mmol) in THF (40 mL), water (20 mL) was added lithium hydroxide (1.2 g, 28.9 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford (S)-2-(5-chloro-1H-indole-2-carboxamido)-3-cyclohexylpropanoic acid (4). TLC system: 50% Ethyl acetate in hexane Rf: 0.2 LCMS (ESI): m/z 349.22 [M+H]⁺

### Methyl 3-(8-acetyl-2-oxo-1,8-diazaspiro[4.5]decan-3-yl)-2-((S)-2-(5-chloro-1H-indole-2-carboxamido)-3-cyclohexylpropanamido)propanoate (5)

To a stirred solution of (S)-2-(5-chloro-1H-indole-2-carboxamido)-3-cyclohexylpropanoic acid (4) (1.2 g, 3.4 mmol) DMF (120 mL) added EDC.HCl (0.98 g, 5.16 mmol), HOBt (0.69 g, 5.16 mmol), DIPEA (1.9 mL, 10.32 mmol) and methyl 3-(8-acetyl-2-oxo-1,8-diazaspiro[4.5]decan-3-yl)-2-aminopropanoate hydrochloride (Int-7) (1.02 g, 3.4 mmol) at 0° C. simultaneously and stirred at room temperature for 16 h. Reaction mixture was diluted with ice water (20 mL), extracted with ethyl acetate (2×60 mL), dried over sodium sulfate and evaporated under reduced pressure. The crude residue was purified by combi-flash NP, compound eluted at 5% methanol in dichloromethane to methyl 3-(8-acetyl-2-oxo-1,8-diazaspiro[4.5]decan-3-yl)-2-((S)-2-(5-chloro-1H-indole-2-carboxamido)-3-cyclohexylpropanamido)propanoate (5). TLC system: 5% Methanol in DCM Rf: 0.2 LCMS (ESI): m/z 628.6 [M+H]⁺

### 3-(8-Acetyl-2-oxo-1,8-diazaspiro[4.5]decan-3-yl)-2-((S)-2-(5-chloro-1H-indole-2-carboxamido)-3-cyclohexylpropanamido)propanoic acid (6)

To a stirred solution of methyl 3-(8-acetyl-2-oxo-1,8-diazaspiro[4.5]decan-3-yl)-2-((S)-2-(5-chloro-1H-indole-2-carboxamido)-3-cyclohexylpropanamido)propanoate (5) (1.2 g, 1.9 mmol) in THF (40 mL), water (20 mL) was added lithium hydroxide (0.24 g, 5.7 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford 3-(8-acetyl-2-oxo-1,8-diazaspiro[4.5]decan-3-yl)-2-((S)-2-(5-chloro-1H-indole-2-carboxamido)-3-cyclohexylpropanamido)propanoic acid (6) TLC system: 10% Methanol in DCM Rf: 0.3 LCMS (ESI): m/z 614.34 [M+H]⁺

### N-((2S)-1-((1-(8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-5-chloro-1H-indole-2-carboxamide (8)

To a stirred solution of 3-(8-acetyl-2-oxo-1,8-diazaspiro[4.5]decan-3-yl)-2-((S)-2-(5-chloro-1H-indole-2-carboxamido)-3-cyclohexylpropanamido)propanoic acid (6) (0.5 g, 0.81 mmol) DCM (20 mL) added HATU (0.464 g, 1.22 mmol), DIPEA (0.45 mL, 2.44 mmol) and 1-(cyanomethyl)tetrahydro-1H-thiophen-1-iumbromide (7) (0.26 g, 1.22 mmol) at 0° C. simultaneously and stirred at room temperature for 2 h. Reaction mixture was diluted with ice water (50 mL), extracted with dichloromethane (2×60 mL), dried over sodium sulfate and evaporated under reduced pressure. The crude residue was purified by combi-flash NP, compound eluted at 5% methanol in dichloromethane to afford N-((2S)-1-((1-(8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-5-chloro-1H-indole-2-carboxamide (8). TLC system: 10% Methanol in dichloromethane Rf: 0.5 LCMS (ESI): m/z 723.5 [M+H]⁺

### N-((2S)-1-((1-(8-acetyl-2-oxo-1,8-diazaspiro[4.5]decan-3-yl)-4-amino-3,4-dioxobutan-2-yl)amino)-3-cyclohexyl-1-oxopropan-2-yl)-5-chloro-1H-indole-2-carboxamide (Compound C126)

To a stirred solution of N-((2S)-1-((1-(8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-5-chloro-1H-indole-2-carboxamide (8) (200 mg, 0.27 mmol) in methanol (3 mL) was added m-CPBA (71.3 mg, 0.41 mmol) at 0° C. and the reaction mixture stirred for 2 h at 0° C. and added aq ammonia (2.0 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford N-((2S)-1-((1-(8-acetyl-2-oxo-1,8-diazaspiro[4.5]decan-3-yl)-4-amino-3,4-dioxobutan-2-yl)amino)-3-cyclohexyl-1-oxopropan-2-yl)-5-chloro-1H-indole-2-carboxamide (Compound C126). TLC system: 10% Methanol in dichloromethane Rf: 0.2 LCMS (ESI): m/z 641.1 [M+H]⁺

### N-((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)-5-chloro-1H-indole-2-carboxamide (6-A)

To a stirred solution of methyl 3-(8-acetyl-2-oxo-1,8-diazaspiro[4.5]decan-3-yl)-2-((S)-2-(5-chloro-1H-indole-2-carboxamido)-3-cyclohexylpropanamido)propanoate (5) (0.500 g, 0.79 mmol) in DCM (10 mL) was added 2M LiBH₄ in THF (0.15 mL, 1.59 mmol) at 0° C. and the reaction mixture stirred for 2 h at 0° C. The progress of the reaction was monitored by TLC and LCMS. Reaction mixture was quenched with saturated ammonium chloride solution (20 mL) and extracted with DCM (2×20 mL). Organic layer was washed with brine solution (30 mL), dried over Na₂SO₄ and concentrated to afford N-((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)-5-chloro-1H-indole-2-carboxamide (6-A). TLC system: 10% Methanol in dichloromethane Rf: 0.4 LCMS (ESI): m/z 600.51 [M+H]⁺

### N-((2S)-1-((1-(8-acetyl-2-oxo-1,8-diazaspiro[4.5]decan-3-yl)-3-oxopropan-2-yl)amino)-3-cyclohexyl-1-oxopropan-2-yl)-5-chloro-1H-indole-2-carboxamide (Compound C141)

To a stirred solution of N-((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)-5-chloro-1H-indole-2-carboxamide (6-A) (170 mg, 0.28 mmol) was dissolved in dichloromethane (10 mL) was added Dess-Martin periodinane (240 mg, 0.566 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford N-((2S)-1-((1-(8-acetyl-2-oxo-1,8-diazaspiro[4.5]decan-3-yl)-3-oxopropan-2-yl)amino)-3-cyclohexyl-1-oxopropan-2-yl)-5-chloro-1H-indole-2-carboxamide (Compound C141). TLC system: 10% Methanol in dichloromethane Rf: 0.5 LCMS (ESI): m/z 598.1 (M+H)⁺

### Example 97: Synthesis of Compound C130

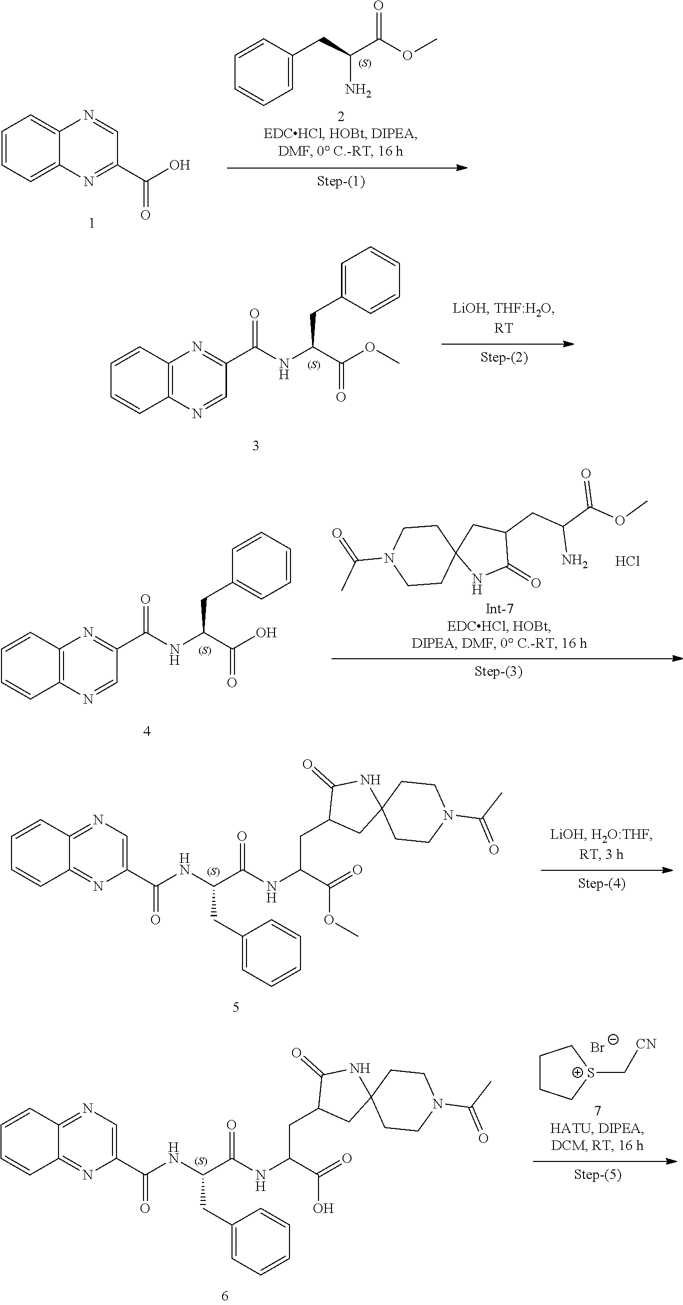

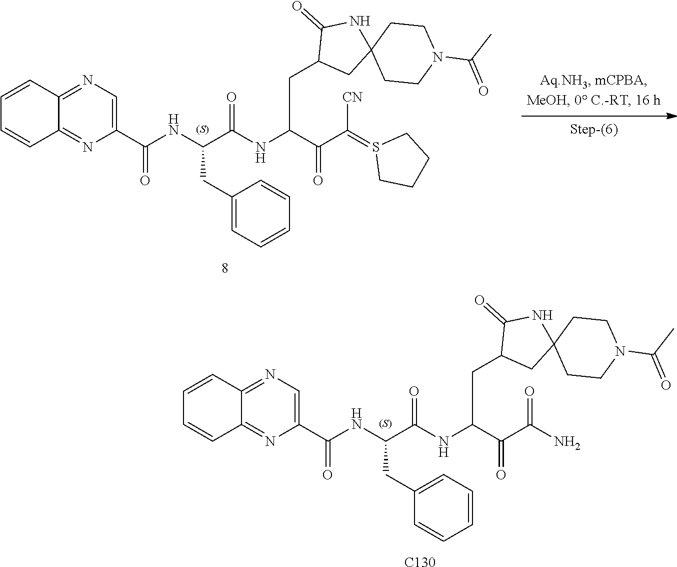

### Methyl (quinoxaline-2-carbonyl)-L-phenylalaninate (3)

To a stirred solution of quinoxaline-2-carboxylic acid (1) (5.0 g, 28.72 mmol) DMF (30 mL) added EDC.HCl (8.22 g, 43.0 mmol), HOBt (5.81 g, 43.00 mmol), DIPEA (15.8 mL, 86.16 mmol) and methyl L-phenylalaninate (2) (5.1 g, 28.72 mmol) at 0° C. simultaneously and stirred at room temperature for 16 h. Reaction mixture was diluted with ice water (80 mL), extracted with ethyl acetate (2×60 mL), dried over sodium sulfate and evaporated under reduced pressure. The crude residue was purified by combi-flash NP, compound eluted at 3% methanol in dichloromethane to methyl (quinoxaline-2-carbonyl)-L-phenylalaninate (3). TLC system: 20% Ethyl acetate in hexane Rf: 0.4 LCMS (ESI): m/z 336.58 [M+H]⁺

### (Quinoxaline-2-carbonyl)-L-phenylalanine (4)

To a stirred solution of methyl (quinoxaline-2-carbonyl)-L-phenylalaninate (3) (5.0 g, 14.9 mmol) in THF (40 mL), water (20 mL) was added lithium hydroxide (1.8 g, 44.7 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford ((quinoxaline-2-carbonyl)-L-phenylalanine (4). TLC system: 50% Ethyl acetate in hexane Rf: 0.2 LCMS (ESI): m/z 322.2 [M+H]⁺

### Methyl 3-(8-acetyl-2-oxo-1,8-diazaspiro[4.5]decan-3-yl)-2-((S)-3-phenyl-2-(quinoxaline-2-carboxamido)propanamido)propanoate (5)

To a stirred solution of ((quinoxaline-2-carbonyl)-L-phenylalanine (4) (1.0 g, 3.1 mmol) DMF (100 mL) added EDC.HCl (0.92 g, 4.66 mmol), HOBt (0.63 g, 4.66 mmol), DIPEA (1.7 mL, 9.33 mmol) and methyl 3-(8-acetyl-2-oxo-1,8-diazaspiro[4.5]decan-3-yl)-2-aminopropanoate hydrochloride (int-7) (0.92 g, 3.1 mmol) at 0° C. simultaneously and stirred at room temperature for 16 h. Reaction mixture was diluted with ice water (20 mL), extracted with ethyl acetate (2×60 mL), dried over sodium sulfate and evaporated under reduced pressure. The crude residue was purified by combi-flash NP, compound eluted at 5% methanol in dichloromethane to methyl 3-(8-acetyl-2-oxo-1,8-diazaspiro[4.5]decan-3-yl)-2-((S)-3-phenyl-2-(quinoxaline-2-carboxamido)propanamido)propanoate (5). TLC system: 5% Methanol in DCM Rf: 0.4 LCMS (ESI): m/z 601.39 [M+H]⁺

### 3-(8-Acetyl-2-oxo-1,8-diazaspiro[4.5]decan-3-yl)-2-((S)-3-phenyl-2-(quinoxaline-2-carboxamido)propanamido)propanoic acid (6)

To a stirred solution of methyl 3-(8-acetyl-2-oxo-1,8-diazaspiro[4.5]decan-3-yl)-2-((S)-3-phenyl-2-(quinoxaline-2-carboxamido)propanamido)propanoate (5) (0.350 g, 0.58 mmol) in THF (20 mL), water (10 mL) was added lithium hydroxide (0.073 g, 1.74 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×10 mL), dried over sodium sulfate, concentrated under reduced pressure to afford 3-(8-acetyl-2-oxo-1,8-diazaspiro[4.5]decan-3-yl)-2-((S)-3-phenyl-2-(quinoxaline-2-carboxamido)propanamido)propanoic acid (6) TLC system: 10% Methanol in DCM Rf: 0.3 LCMS (ESI): m/z 587.62[M+H]⁺

### N-((2S)-1-((1-(8-acetyl-2-oxo-1,8-diazaspiro[4.5]decan-3-yl)-4-cyano-3-oxo-4-(tetrahydro-1λ⁴-thiophen-1-ylidene)butan-2-yl)amino)-1-oxo-3-phenylpropan-2-yl)quinoxaline-2-carboxamide (8)

To a stirred solution of 3-(8-acetyl-2-oxo-1,8-diazaspiro[4.5]decan-3-yl)-2-((S)-3-phenyl-2-(quinoxaline-2-carboxamido)propanamido)propanoic acid (6) (0.2 g, 0.34 mmol) DCM (20 mL) added HATU (0.194 g, 0.51 mmol), DIPEA (0.2 mL, 1.02 mmol) and 1-(cyanomethyl)tetrahydro-1H-thiophen-1-iumbromide (7) (0.110 g, 1.22 mmol) at 0° C. simultaneously and stirred at room temperature for 2 h. Reaction mixture was diluted with ice water (50 mL), extracted with dichloromethane (2×10 mL), dried over sodium sulfate and evaporated under reduced pressure. The crude residue was purified by combi-flash NP, compound eluted at 5% methanol in dichloromethane to afford N-((2S)-1-((1-(8-acetyl-2-oxo-1,8-diazaspiro[4.5]decan-3-yl)-4-cyano-3-oxo-4-(tetrahydro-1λ⁴-thiophen-1-ylidene)butan-2-yl)amino)-1-oxo-3-phenylpropan-2-yl)quinoxaline-2-carboxamide (8). TLC system: 10% Methanol in dichloromethane Rf: 0.5 LCMS (ESI): m/z 696.6 [M+H]⁺

### N-((2S)-1-((1-(8-acetyl-2-oxo-1,8-diazaspiro[4.5]decan-3-yl)-4-amino-3,4-dioxobutan-2-yl)amino)-1-oxo-3-phenylpropan-2-yl)quinoxaline-2-carboxamide (Compound C130)

To a stirred solution of N-((2S)-1-((1-(8-acetyl-2-oxo-1,8-diazaspiro[4.5]decan-3-yl)-4-cyano-3-oxo-4-(tetrahydro-1λ⁴-thiophen-1-ylidene)butan-2-yl)amino)-1-oxo-3-phenylpropan-2-yl)quinoxaline-2-carboxamide (8) (0.170 g, 0.24 mmol) in methanol (3 mL) was added m-CPBA (0.084 mg, 0.48 mmol) at 0° C. and the reaction mixture stirred for 2 h at 0° C. and added aq ammonia (2.0 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford N-((2S)-1-((1-(8-acetyl-2-oxo-1,8-diazaspiro[4.5]decan-3-yl)-4-amino-3,4-dioxobutan-2-yl)amino)-1-oxo-3-phenylpropan-2-yl)quinoxaline-2-carboxamide (Compound C130). TLC system: 10% Methanol in dichloromethane Rf: 0.2 LCMS (ESI): m/z 641.1 [M+H]⁺

### Example 98: Synthesis of Compounds C135 and C137

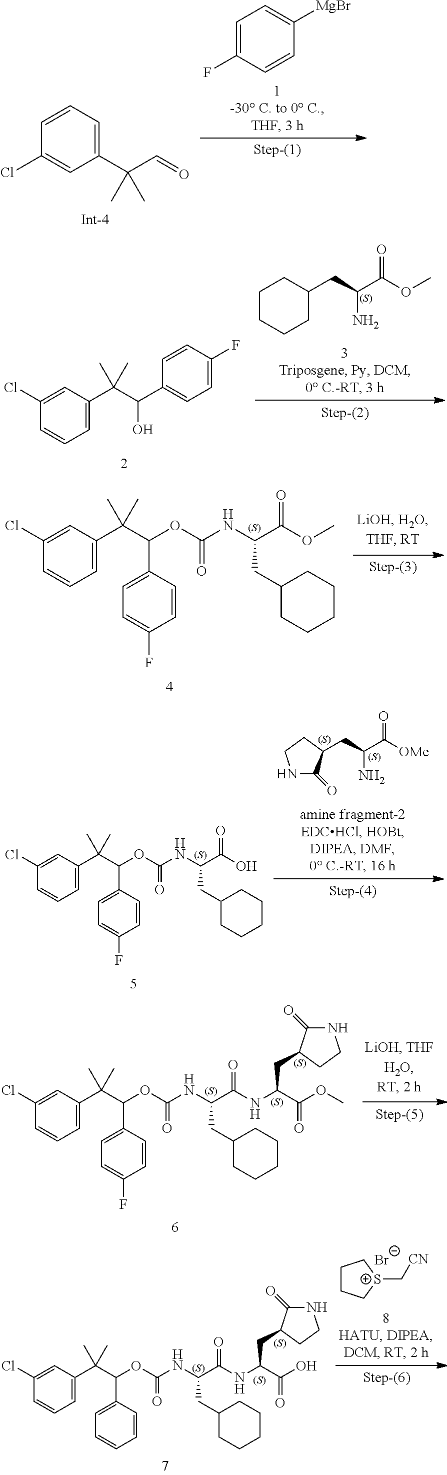

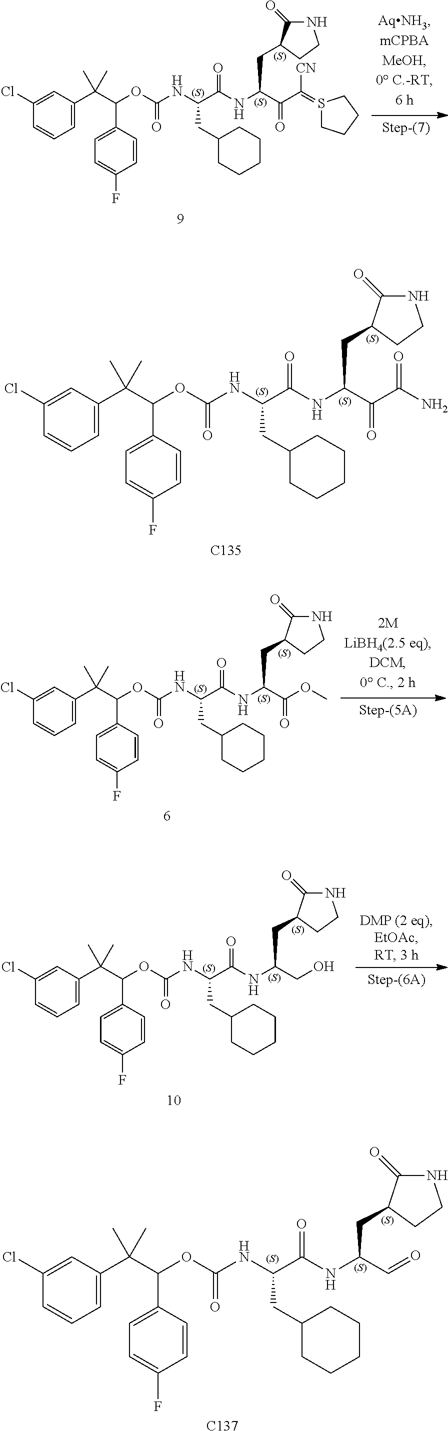

### 2-(3-Chlorophenyl)-1-(4-fluorophenyl)-2-methylpropan-1-ol (2)

To a stirred solution of 2-(3-chlorophenyl)-2-methylpropanal (Int-4) (5 g, 27.472 mmol) in THF (50 mL) was added 3-methyl magnesium bromide (82 mL, 82.417 mmol) at −30° C. and the reaction mixture was stirred at 0° C. for 3 h. The progress of the reaction was monitored by TLC. The reaction mixture was quenched with saturated NH₄Cl (50 mL) and filtered through celite pad and washed with ethyl acetate (100 mL), dried over sodium sulfate and evaporated under reduced pressure. The crude residue was purified by silica gel column by eluting with 10% ethyl acetate in hexane to afford 2-(3-chlorophenyl)-1-(4-fluorophenyl)-2-methylpropan-1-ol (2). TLC system: 10% Ethyl acetate in hexane Rf: 0.3 LCMS (ESI): m/z=261.21 [M−OH]

### Methyl (2S)-2-(((2-(3-chlorophenyl)-1-(4-fluorophenyl)-2-methylpropoxy) carbonyl) amino)-3-cyclohexylpropanoate (4)

To a stirred solution of 2-(3-chlorophenyl)-1-(4-fluorophenyl)-2-methylpropan-1-ol (2) (6.5 g, 23.381 mmol) in DCM (65 mL) was added pyridine (6.5 mL) methyl L-leucinate hydrochloride (3) (5.1 g, 28.057 mmol) followed by triphosgene (3.46 g, 11.690 mmol) at 0° C. slowly and stirred at RT for 16 h. The progress of the reaction was monitored by TLC. Reaction mixture was quenched with 1N aq. HCl (100 mL) then extracted with DCM (2×50 mL), washed with 2×50 mL NaHCO₃ solution the organic layer was dried over sodium sulfate and evaporated under reduced pressure. The crude residue was purified by silica gel column by eluting with 15% ethyl acetate in hexane to afford methyl (2S)-2-(((2-(3-chlorophenyl)-1-(4-fluorophenyl)-2-methylpropoxy) carbonyl) amino)-3-cyclohexylpropanoate (4). TLC system: 10% Ethyl acetate in hexane Rf: 0.3 LCMS (ESI): m/z=512.18 [M+Na]⁺

### (2S)-2-(((2-(3-chlorophenyl)-1-(4-fluorophenyl)-2-methylpropoxy) carbonyl) amino)-3-cyclohexylpropanoic acid (5)

To a stirred solution of methyl (2S)-2-(((2-(3-chlorophenyl)-1-(4-fluorophenyl)-2-methylpropoxy)carbonyl) amino)-3-cyclohexylpropanoate (4) (5.5 g, 11.247 mmol) in THF (40 mL), water (10 mL) was added LiOH.H₂O (809 mg, 33.742 mmol) at 0° C. Reaction mixture was stirred at RT for 3 h. The progress of the reaction was monitored by TLC. After consumption of starting material, the reaction mixture was concentrated and acidified with 1N HCl, extracted with ethyl acetate (2×50 mL), dried over sodium sulfate and evaporated under reduced pressure to afford (2S)-2-(((2-(3-chlorophenyl)-1-(4-fluorophenyl)-2-methylpropoxy)carbonyl)amino)-3-cyclohexylpropanoic acid (5) which was used directly in the next step. TLC system: 10% Methanol in dichloromethane Rf: 0.2 LCMS (ESI): m/z=498.45 [M+Na]⁺

### Methyl (2S)-2-((2S)-2-(((2-(3-chlorophenyl)-1-(4-fluorophenyl)-2-methylpropoxy) carbonyl) amino)-3-cyclohexylpropanamido)-3-((S)-2-oxopyrrolidin-3-yl) propanoate (6)

To a stirred solution of ((1-(3-(benzyloxy)benzyl)cyclopropoxy)carbonyl)-L-leucine (5) (1 g, 2.105 mmol) in DMF (20 mL) was added EDC.HCl (426 mg, 3.157 mmol), HOBT (603 mg, 3.157 mmol), DIPEA (1.16 mL, 6.315 mmol) and methyl (S)-2-amino-3-((S)-2-oxopyrrolidin-3-yl)propanoate (amine-fragment-2) (0.587 g, 3.157 mmol) at 0° C. simultaneously and stirred at room temperature for 16 h. Reaction mixture was diluted with ice water (50 mL), extracted with ethyl acetate (2×50 mL), the organic layer was dried over sodium sulfate and evaporated under reduced pressure to afford crude compound. The crude residue was purified by silica gel column by eluting with 80% Ethyl acetate and pet-ether to afford methyl (2S)-2-((2S)-2-(((2-(3-chlorophenyl)-1-(4-fluorophenyl)-2-methylpropoxy)carbonyl)amino)-3-cyclohexyl propanamido)-3-((S)-2-oxopyrrolidin-3-yl) propanoate (6). TLC system: 10% Methanol in dichloromethane Rf: 0.3 LCMS (ESI): m/z=644.51 [M+H]⁺

### (2S)-2-((2S)-2-(((2-(3-chlorophenyl)-1-(4-fluorophenyl)-2-methylpropoxy) carbonyl) amino)-3-cyclohexylpropanamido)-3-((S)-2-oxopyrrolidin-3-yl) propanoic acid (7)

To a stirred solution of methyl (2S)-2-((2S)-2-(((2-(3-chlorophenyl)-1-(4-fluorophenyl)-2-methylpropoxy)carbonyl)amino)-3-cyclohexylpropanamido)-3-((S)-2-oxopyrrolidin-3-yl) propanoate (6) (800 mg, 1.244 mmol) in THF (10 mL), water (5 mL) was added lithium hydroxide (89 mg, 3.732 mmol) at RT and stirred at room temperature for 2 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-((2S)-2-(((2-(3-chlorophenyl)-1-(4-fluorophenyl)-2-methylpropoxy)carbonyl)amino)-3-cyclohexyl propanamido)-3-((S)-2-oxopyrrolidin-3-yl) propanoic acid (7). TLC system: 15% Methanol in dichloromethane Rf: 0.2 LCMS (ESI): m/z 630.28 [M+H]⁺

### 2-(3-Chlorophenyl)-1-(4-fluorophenyl)-2-methylpropyl ((S)-1-(((S)-4-cyano-3-oxo-1-((S)-2-oxopyrrolidin-3-yl)-4-(tetrahydro-1l4-thiophen-1-ylidene) butan-2-yl) amino)-3-cyclohexyl-1-oxopropan-2-yl)carbamate (9)

To a stirred solution of (2S)-2-((2S)-2-(((2-(3-chlorophenyl)-1-(4-fluorophenyl)-2-methylpropoxy)carbonyl)amino)-3-cyclohexylpropanamido)-3-((S)-2-oxopyrrolidin-3-yl) propanoic acid (7) (750 mg, 1.192 mmol) DCM (10 mL) added HATU (679 mg, 1.78 mmol), DIPEA (0.6 mL, 3.577 mmol) and 1-(cyanomethyl)tetra hydro-1H-thiophen-1-iumbromide (8) (228 mg, 1.788 mmol) at 0° C. simultaneously at 0° C. and stirred at room temperature at RT for 2 h. Reaction mixture was quenched with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 2-(3-chlorophenyl)-1-(4-fluoro phenyl)-2-methylpropyl((S)-1-(((S)-4-cyano-3-oxo-1-((S)-2-oxopyrrolidin-3-yl)-4-(tetrahydro-1l4-thiophen-1-ylidene) butan-2-yl)amino)-3-cyclohexyl-1-oxopropan-2-yl) carbamate (9). TLC system: 10% Methanol in dichloromethane Rf: 0.4 LCMS (ESI): m/z 739.31 [M+Na]⁺

### 2-(3-Chlorophenyl)-1-(4-fluorophenyl)-2-methylpropyl ((S)-1-(((S)-4-amino-3,4-dioxo-1-((S)-2-oxopyrrolidin-3-yl) butan-2-yl) amino)-3-cyclohexyl-1-oxopropan-2-yl) carbamate (Compound C135)

To a stirred solution of 2-(3-chlorophenyl)-1-(4-fluorophenyl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (9) (200 mg, 0.271 mmol) in methanol (3 mL) was added mCPBA (93 mg, 0.542 mmol) at 0° C. and the reaction mixture stirred for 2 h at 0° C. and added aq ammonia (2 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over sodium sulfate and concentrated to get crude compound. The crude compound was purified by prep-HPLC to afford 2-(3-chlorophenyl)-1-(4-fluorophenyl)-2-methylpropyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C135). TLC system: 10% Methanol in dichloromethane Rf: 0.3 LCMS (ESI): m/z 657.2 [M+H]+

### Methyl (2S)-2-((2S)-2-(((2-(3-chlorophenyl)-1-(4-fluorophenyl)-2-methylpropoxy) carbonyl) amino)-3-cyclohexylpropanamido)-3-((S)-2-oxopyrrolidin-3-yl) propanoate (10)

To a stirred solution of methyl (2S)-2-((2S)-2-(((2-(3-chlorophenyl)-1-(4-fluorophenyl)-2-methylpropoxy)carbonyl)amino)-3-cyclohexylpropanamido)-3-((S)-2-oxopyrrolidin-3-yl)propanoate (6) (1.5 g, 2.439 mmol) in DCM (10 mL) was added 2M LiBH₄ in THF (2.4 mL, 4.878 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to get compound to afford 2-(3-chlorophenyl)-2-methyl-1-(m-tolyl)propyl ((S)-3-cyclohexyl-1-(((S)-1-hydroxy-3-((S)-2-oxopyrrolidin-3-yl) propan-2-yl)amino)-1-oxopropan-2-yl) carbamate (10). TLC system: 10% Methanol in DCM Rf: 0.4 LCMS (ESI): m/z 616.69 (M+H)⁺

### 2-(3-Chlorophenyl)-1-(4-fluorophenyl)-2-methylpropyl ((S)-3-cyclohexyl-1-oxo-1-(((S)-1-oxo-3-((S)-2-oxopyrrolidin-3-yl) propan-2-yl) amino) propan-2-yl) carbamate (Compound C137)

To a stirred solution of 2-(3-chlorophenyl)-2-methyl-1-(m-tolyl)propyl ((S)-3-cyclohexyl-1-(((S)-1-hydroxy-3-((S)-2-oxopyrrolidin-3-yl) propan-2-yl)amino)-1-oxopropan-2-yl)carbamate (10) (300 mg, 0.487 mmol) was dissolved in ethyl acetate (10 mL) was added Dess-Martin periodinane (620 mg, 1.463 mmol) at 0° C. and stirred at RT for 3 h. Reaction mixture was diluted with ethyl acetate (20 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by combi-flash chromatography by eluting 3% methanol in dichloromethane to afford 2-(3-chlorophenyl)-1-(4-fluorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (Compound C137). TLC system: 10% Methanol in DCM Rf: 0.5 LCMS (ESI): m/z 614.2 (M+H)⁺

### Example 99: Synthesis of Compounds C117 and C133

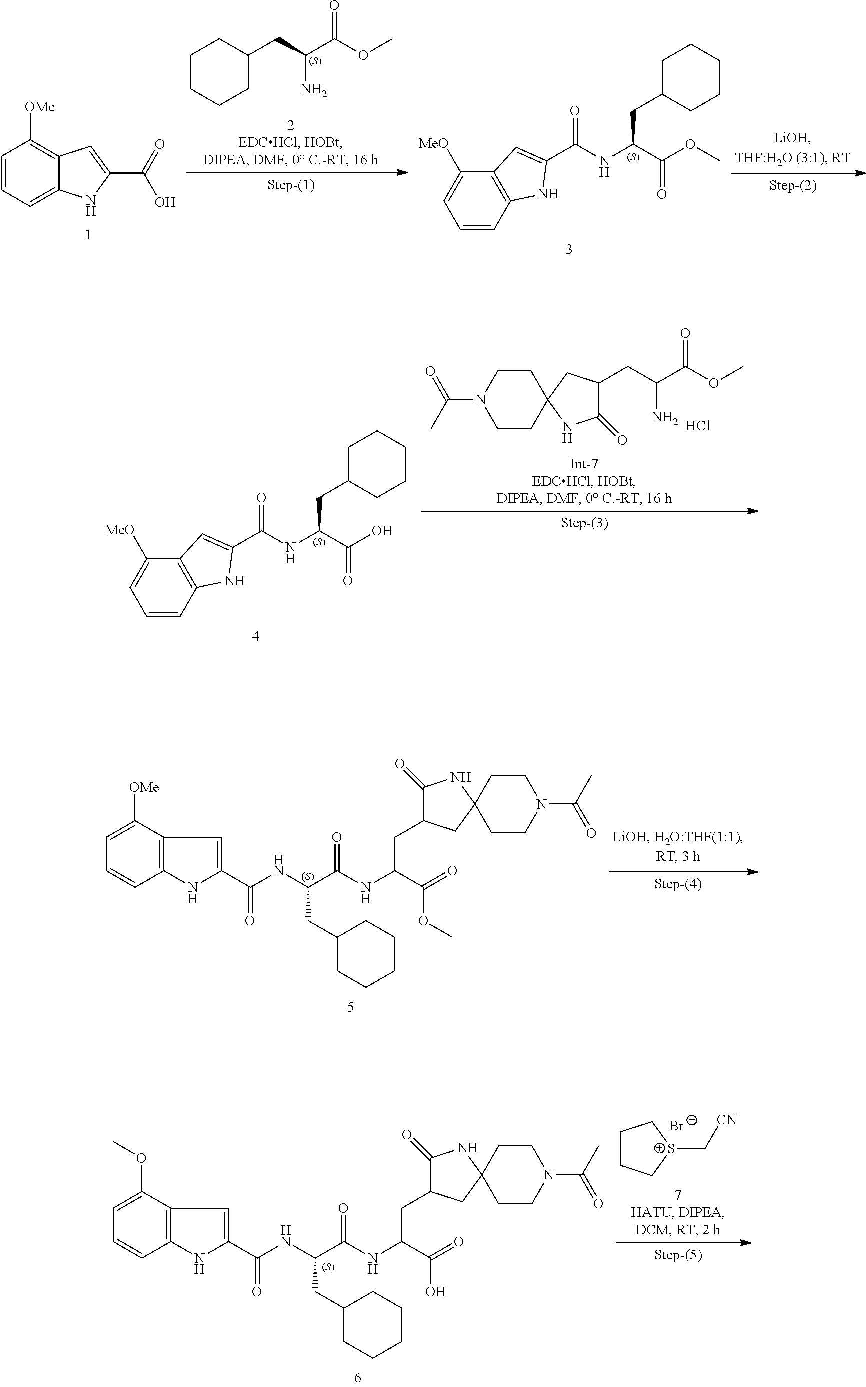

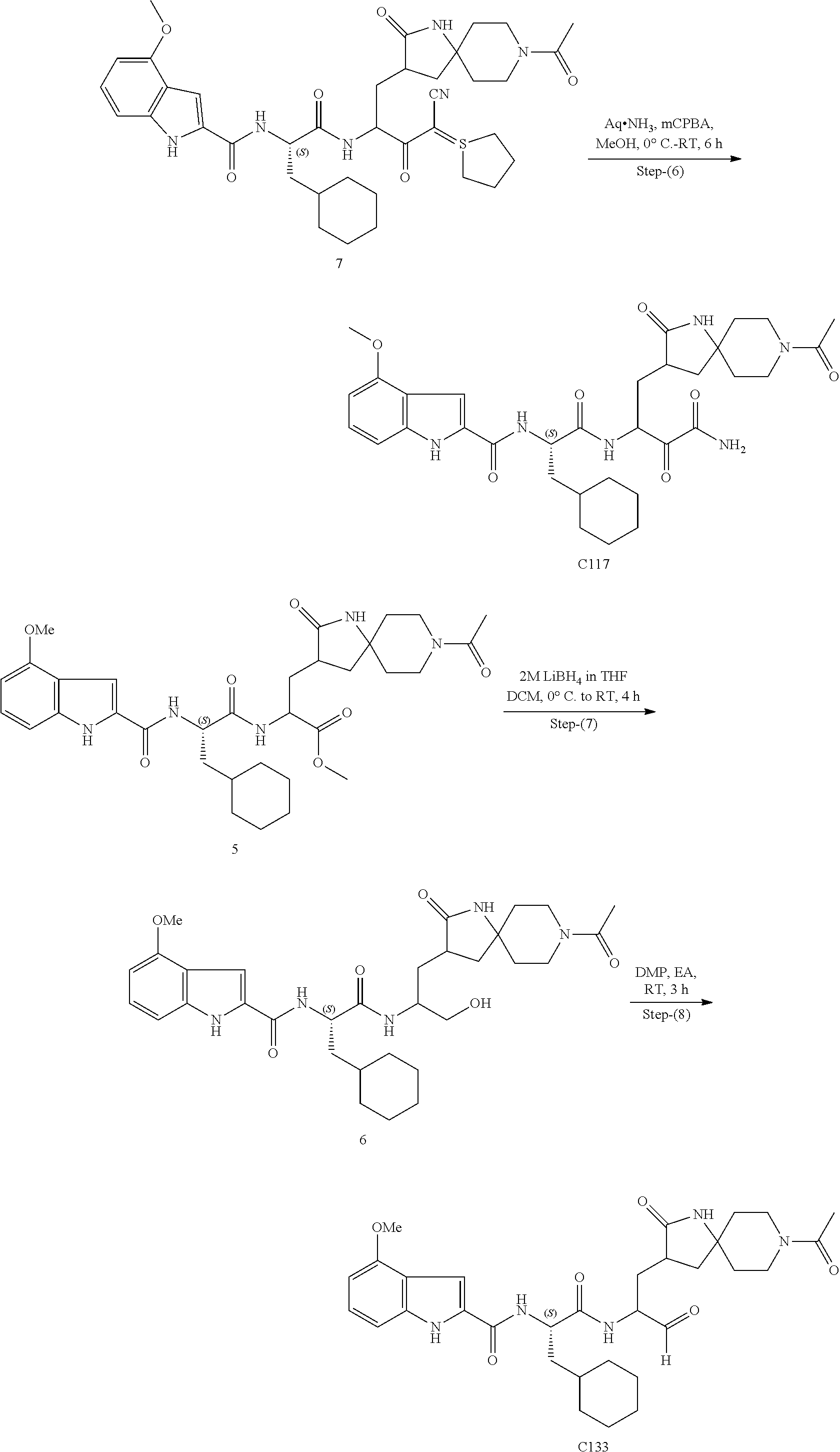

### Methyl (S)-3-cyclohexyl-2-(4-methoxy-1H-indole-2-carboxamido)propanoate (3)

To a stirred solution of 4-Methoxy-1H-Indole-2-Carboxylic acid (1) (3 g 157 mmol) in DMF (30 mL) at 0° C. was added EDC.HCl (4.4 g, 23 mmol), HOBT (3.10 g, 23 mmol), DIPEA (8.3 g, 47 mmol) and Methyl (S)-2-amino-3-cyclohexylpropanoate hydrochloride (2) (4.16 g, 18 mmol) simultaneously and stirred at room temperature for 16 h. The progress of the reaction was monitored by TLC and LCMS. After 16 h, reaction mixture was quenched with ice water (50 mL), extracted with ethyl acetate (2×40 mL), the combined organic layer was dried over sodium sulfate and evaporated under reduced pressure. The crude residue was purified by silica gel column by eluting with 50% ethyl acetate in pet ether to afford methyl (S)-3-cyclohexyl-2-(4-methoxy-1H-indole-2-carboxamido)propanoate (3). TLC system: 40% Ethyl acetate in Pet ether Rf: 0.5 LCMS (ESI): m/z 359.5 [M+H]⁺

### (S)-3-cyclohexyl-2-(4-methoxy-1H-indole-2-carboxamido) propanoic acid (4)

To a stirred solution of methyl (S)-3-cyclohexyl-2-(4-methoxy-1H-indole-2-carboxamido) propanoate (3) (5 g, 13.9 mmol) in THF (30 mL), water (20 mL) was added lithium hydroxide (1.75 g, 41.8 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×30 mL), dried over sodium sulfate, concentrated under reduced pressure to afford (S)-3-cyclohexyl-2-(4-methoxy-1H-indole-2-carboxamido) propanoic acid (4). TLC system: 80% Ethyl acetate in Pet ether Rf: 0.2 LCMS (ESI): m/z 345.3 [M+H]⁺

### Methyl 3-(8-acetyl-2-oxo-1,8-diazaspiro[4.5]decan-3-yl)-2-((S)-3-cyclohexyl-2-(4-methoxy-1H-indole-2-carboxamido)propanamido)propanoate (5)

To a stirred solution of (S)-3-cyclohexyl-2-(4-methoxy-1H-indole-2-carboxamido) propanoic acid (4) (1 g, 2.9 mmol) in DMF (10 mL) at 0° C. was added EDC.HCl (832 mg, 4.3 mmol), HOBT (588 mg, 4.3 mmol), DIPEA (1.54 mL, 8.7 mmol) and methyl 3-(8-acetyl-2-oxo-1,8-diazaspiro[4.5]decan-3-yl)-2-aminopropanoate hydrochloride (Int-7) (1.06 g, 3.1 mmol) simultaneously and stirred at room temperature for 16 h. The progress of the reaction was monitored by TLC and LCMS. After 16 h, reaction mixture was quenched with ice water (30 mL), extracted with ethyl acetate (2×50 mL), the combined organic layer was dried over sodium sulfate and evaporated under reduced pressure. The crude residue was purified by silica gel column by eluting with 5% Methanol in Dichloromethane to afford Methyl 3-(8-acetyl-2-oxo-1,8-diazaspiro[4.5]decan-3-yl)-2-((S)-3-cyclohexyl-2-(4-methoxy-1H-indole-2-carboxamido)propanamido)propanoate (5). TLC system: 10% Methanol in DCM R_{f}: 0.65 LCMS (ESI): m/z 624.71 (M+H)⁺

### 3-(8-Acetyl-2-oxo-1,8-diazaspiro[4.5]decan-3-yl)-2-((S)-3-cyclohexyl-2-(4-methoxy-1H-indole-2-carboxamido)propanamido)propanoic acid (6)

To a stirred solution of methyl 3-(8-acetyl-2-oxo-1,8-diazaspiro[4.5]decan-3-yl)-2-((S)-3-cyclohexyl-2-(4-methoxy-1H-indole-2-carboxamido)propanamido)propanoate (5) (600 mg, 0.93 mmol) in THF (4 mL), water (2 mL) was added lithium hydroxide (121 mg, 0.96 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×30 mL), dried over sodium sulfate, concentrated under reduced pressure to afford 3-(8-acetyl-2-oxo-1,8-diazaspiro[4.5]decan-3-yl)-2-((S)-3-cyclohexyl-2-(4-methoxy-1H-indole-2-carboxamido)propanamido)propanoic acid (6). TLC system: 10% Methanol in Dichloromethane Rf: 0.2 LCMS (ESI): m/z 610.6 [M+H]⁺

### N-((2S)-1-((1-(8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-4-methoxy-1H-indole-2-carboxamide (8)

To a stirred solution of 3-(8-acetyl-2-oxo-1,8-diazaspiro[4.5]decan-3-yl)-2-((S)-3-cyclohexyl-2-(4-methoxy-1H-indole-2-carboxamido)propanamido)propanoic acid (6) (400 mg, 6.5 mmol) in DCM (4 mL) added HATU (374 mg, 9.8 mmol), DIPEA (0.35 mL, 1.97 mmol) and 1-(cyanomethyl)tetra hydro-1H-thiophen-1-iumbromide (7) (202 mg, 9.8 mmol) at 0° C. simultaneously and stirred at room temperature for 2 h. Reaction mixture was diluted with ice water (15 mL), extracted with dichloromethane (2×20 mL), dried over sodium sulfate and evaporated under reduced pressure. The crude residue was purified by combi-flash NP, compound eluted at 5% methanol in dichloromethane to N-((2S)-1-((1-(8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-4-methoxy-1H-indole-2-carboxamide (8). TLC system: 10% Methanol in dichloromethane Rf: 0.4 LCMS (ESI): m/z 719.7 [M+H]⁺

### N-((2S)-1-((1-(8-Acetyl-2-oxo-1,8-diazaspiro[4.5]decan-3-yl)-4-amino-3,4-dioxobutan-2-yl)amino)-3-cyclohexyl-1-oxopropan-2-yl)-4-methoxy-1H-indole-2-carboxamide (Compound C117)

To a stirred solution of N-((2S)-1-((1-(8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-4-methoxy-1H-indole-2-carboxamide (8) (190 mg, 0.26 mmol) in methanol (3 mL) was added m-CPBA (113 mg, 0.66 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (20 mL) and extracted with DCM (2×15 mL). Organic layer was washed with brine solution (20 mL), dried over Na₂SO₄ and concentrated to get crude compound. The crude compound was purified by prep HPLC to afford N-((2S)-1-((1-(8-acetyl-2-oxo-1,8-diazaspiro[4.5]decan-3-yl)-4-amino-3,4-dioxobutan-2-yl)amino)-3-cyclohexyl-1-oxopropan-2-yl)-4-methoxy-1H-indole-2-carboxamide (Compound C117). TLC system: 10% Methanol in dichloromethane Rf: 0.3 LCMS (ESI): m/z 637.3 [M+H]⁺

### N-((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)-4-methoxy-1H-indole-2-carboxamide (6)

To a stirred solution of methyl 3-(8-acetyl-2-oxo-1,8-diazaspiro[4.5]decan-3-yl)-2-((S)-3-cyclohexyl-2-(4-methoxy-1H-indole-2-carboxamido)propanamido)propanoate (5) (250 mg, 0.4 mmol) in DCM (10 mL) was added 2M LiBH₄ in THF (0.6 mL, 1.2 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford the N-((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)-4-methoxy-1H-indole-2-carboxamide (6). TLC system: 5% MeOH in DCM R_{f} 0.3 LCMS (ESI): m/z 596.73 (M+H)⁺

### N-((2S)-1-((1-(8-acetyl-2-oxo-1,8-diazaspiro[4.5]decan-3-yl)-3-oxopropan-2-yl)amino)-3-cyclohexyl-1-oxopropan-2-yl)-4-methoxy-1H-indole-2-carboxamide (Compound C133)

To a stirred solution of N-((2S)-1-((1-(8-acetyl-2-oxo-1,8-diazaspiro[4.5]decan-3-yl)-3-hydroxypropan-2-yl)amino)-3-cyclohexyl-1-oxopropan-2-yl)-4-methoxy-1H-indole-2-carboxamide (6) (6) (200 mg, 0.35 mmol) in ethyl acetate (5 mL) was added Dess-Martin periodinane (445 mg, 1.05 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford N-((2S)-1-((1-(8-acetyl-2-oxo-1,8-diazaspiro[4.5]decan-3-yl)-3-oxopropan-2-yl)amino)-3-cyclohexyl-1-oxopropan-2-yl)-4-methoxy-1H-indole-2-carboxamide (Compound C133). TLC system: 5% Methanol in DCM R_{f}: 0.4 LCMS (ESI): m/z 594.2 (M+H)⁺

### Example 100: Synthesis of Compound C121

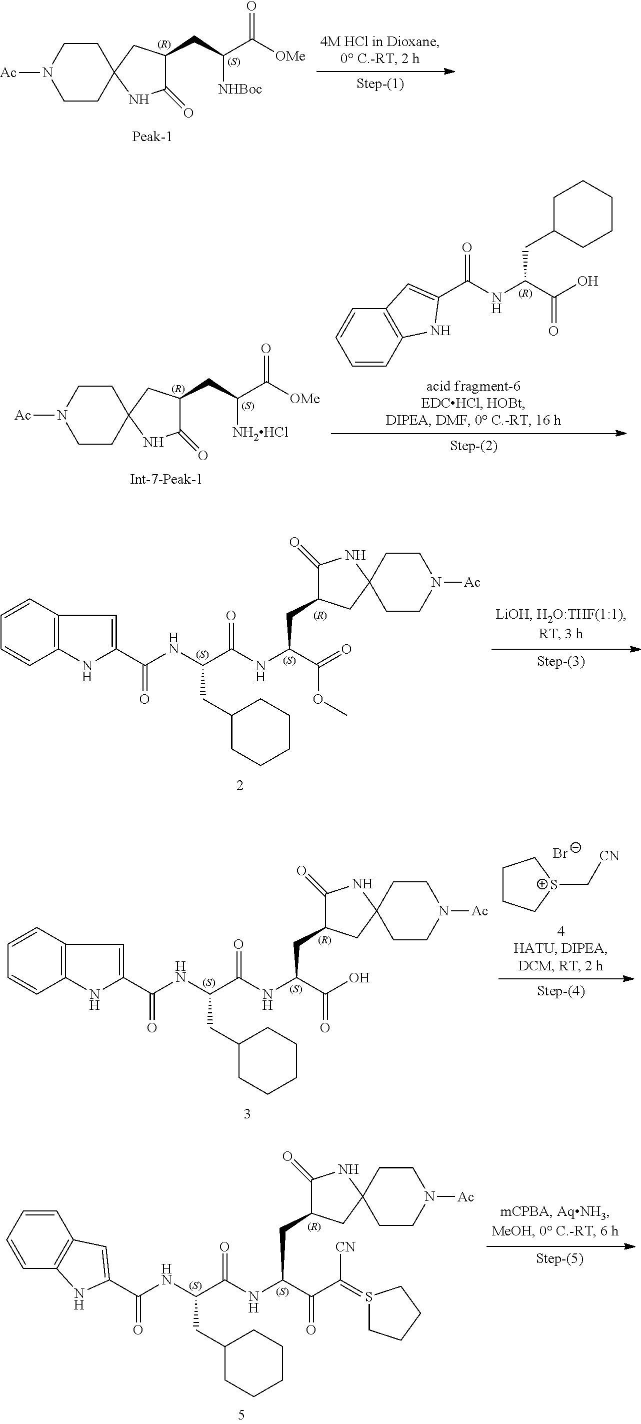

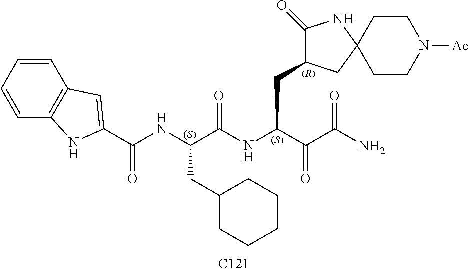

### Methyl (S)-3-((R)-8-acetyl-2-oxo-1,8-diazaspiro[4.5]decan-3-yl)-2-aminopropanoate hydrochloride (Int-7-Peak-1)

To the stirred solution of methyl 3-(8-acetyl-2-oxo-1,8-diazaspiro[4.5]decan-3-yl)-2-((tert-butoxycarbonyl)amino)propanoate (Peak-1) (1.1 g, 2.77 mmol) in DCM (4 mL), added dioxane HCl (4 mL) at 0° C. and stirred at room temperature for 2 h. The progress of the reaction was monitored by TLC and LCMS after 2 h, reaction mixture was evaporated under reduced pressure. The crude residue was purified by trituration with n-pentane afforded methyl (S)-3-((S)-8-acetyl-2-oxo-1,8-diazaspiro[4.5]decan-3-yl)-2-aminopropanoate hydrochloride (Int-7 Peak-1). TLC system: 10% methanol in dichloromethane Rf: 0.1 LCMS (ESI): m/z 298.08 [M+H]⁺

### Methyl (S)-3-((R)-8-acetyl-2-oxo-1,8-diazaspiro[4.5]decan-3-yl)-2-((S)-3-cyclohexyl-2-(1H-indole-2-carboxamido)propanamido)propanoate (2)

To a stirred solution of (R)-3-cyclohexyl-2-(1H-indole-2-carboxamido) propanoic acid (Amine fragment-6) (700 mg, 3.92 mmol) in DMF (5 mL) at 0° C. was added EDC.HCl (630 mg, 3.3 mmol), HOBT (440 mg, 3.3 mmol), DIPEA (1.1 ml, 4.5 mmol) and methyl (S)-3-((R)-8-acetyl-2-oxo-1,8-diazaspiro[4.5]decan-3-yl)-2-aminopropanoate hydrochloride (Int-7 Peak-1) (840 mg, 1.2 mmol) simultaneously and stirred at room temperature for 16 h. The progress of the reaction was monitored by TLC and LCMS. After 16 h, reaction mixture was quenched with ice water (50 mL), extracted with ethyl acetate (2×15 mL), the combined organic layer was dried over sodium sulfate and evaporated under reduced pressure. The crude residue was purified by silica gel column by eluting with 10% methanol in dichloromethane to afford methyl (S)-3-((R)-8-acetyl-2-oxo-1,8-diazaspiro[4.5]decan-3-yl)-2-((S)-3-cyclohexyl-2-(1H-indole-2-carboxamido)propanamido)propanoate (2). TLC system: 10% methanol in dichloromethane Rf: 0.5 LCMS (ESI): m/z 594.6 [M+H]⁺

### (S)-3-((R)-8-acetyl-2-oxo-1,8-diazaspiro[4.5]decan-3-yl)-2-((S)-3-cyclohexyl-2-(1H-indole-2-carboxamido)propanamido)propanoic acid (3)

To a stirred solution of methyl (S)-3-((R)-8-acetyl-2-oxo-1,8-diazaspiro[4.5]decan-3-yl)-2-((S)-3-cyclohexyl-2-(1H-indole-2-carboxamido)propanamido)propanoate (2) (550 mg, 0.927 mmol) in THF (5 mL), water (3 mL) was added lithium hydroxide (66 mg, 2.782 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜2 and extracted with 10% MeOH in DCM (2×20 mL), dried over sodium sulfate, concentrated under reduced pressure to afford (S)-3-((R)-8-acetyl-2-oxo-1,8-diazaspiro[4.5]decan-3-yl)-2-((S)-3-cyclohexyl-2-(1H-indole-2-carboxamido)propanamido)propanoic acid (2). TLC system: 10% MeOH in DCM Rf: 0.2 LCMS (ESI): m/z 580.45 [M+H]⁺

### N—((S)-1-(((S)-1-((R)-8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-1H-indole-2-carboxamide (5)

To a stirred solution of (S)-3-((R)-8-acetyl-2-oxo-1,8-diazaspiro[4.5]decan-3-yl)-2-((S)-3-cyclohexyl-2-(1H-indole-2-carboxamido)propanamido)propanoic acid (3) (300 mg, 0.518 mmol) in THF (5 mL) added HATU (269 mg, 0.777 mmol), DIPEA (0.26 mL, 1.165 mmol) and 1-(cyanomethyl)tetrahydro-1H-thiophen-1-ium (4) (160 mg, 0.777 mmol) at 0° C. simultaneously and stirred at room temperature for 2 h. Reaction mixture was diluted with ice water (25 mL), extracted with EtoAc (2×15 mL), dried over sodium sulfate and evaporated under reduced pressure. The crude residue was purified by combi-flash NP, compound eluted at 5% methanol in dichloromethane to afford N—((S)-1-(((S)-1-((R)-8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-1H-indole-2-carboxamide (5). TLC system: 10% Methanol in dichloromethane Rf: 0.4 LCMS (ESI): m/z 689.61 [M+H]⁺

### N—((S)-1-(((S)-1-((R)-8-acetyl-2-oxo-1,8-diazaspiro[4.5]decan-3-yl)-4-amino-3,4-dioxobutan-2-yl)amino)-3-cyclohexyl-1-oxopropan-2-yl)-1H-indole-2-carboxamide (Compound C121)

To a stirred solution of N—((S)-1-(((S)-1-((R)-8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-1H-indole-2-carboxamide (5) (200 mg, 0.290 mmol) in methanol (3 mL) was added mCPBA (124 mg, 0.726 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep-HPLC to afford N—((S)-1-(((S)-1-((R)-8-acetyl-2-oxo-1,8-diazaspiro[4.5]decan-3-yl)-4-amino-3,4-dioxobutan-2-yl)amino)-3-cyclohexyl-1-oxopropan-2-yl)-1H-indole-2-carboxamide (Compound C121). TLC system: 10% Methanol in dichloromethane Rf: 0.3 LCMS (ESI): m/z 607.2 [M+H]⁺

### Example 101: Synthesis of Compound C125

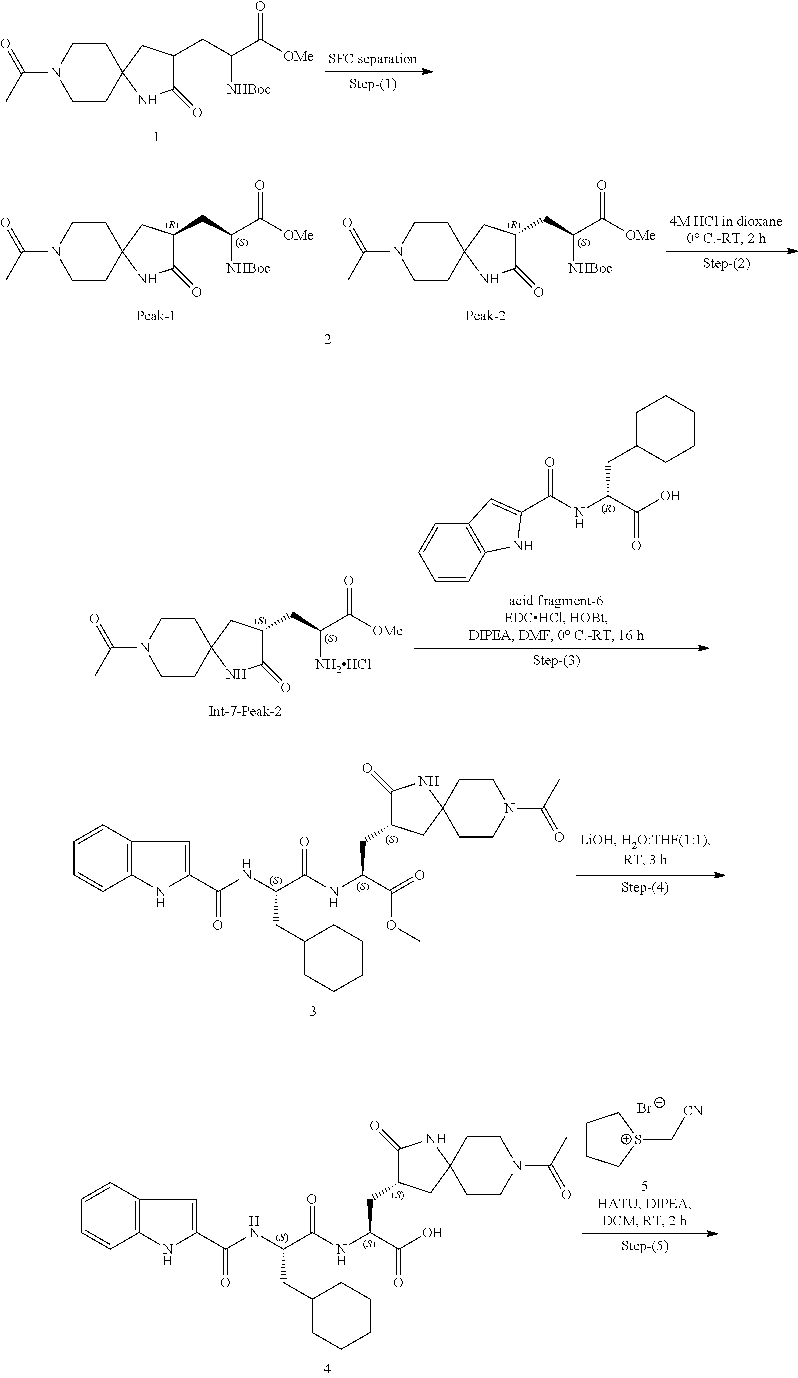

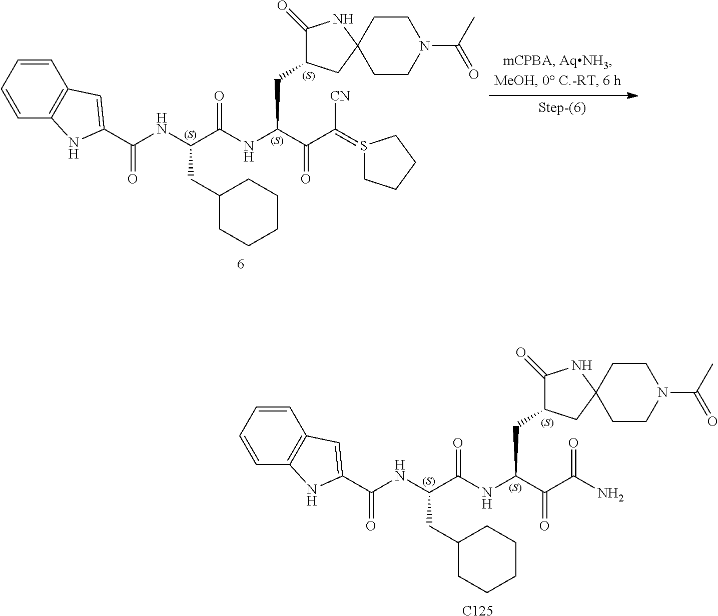

### Methyl (R)-3-((R)-8-acetyl-2-oxo-1,8-diazaspiro[4.5]decan-3-yl)-2-((tert-butoxycarbonyl)amino)propanoate (2) (Peak-1) & Methyl (S)-3-((S)-8-acetyl-2-oxo-1,8-diazaspiro[4.5]decan-3-yl)-2-((tert-butoxycarbonyl)amino)propanoate (2) (Peak-2)

Methyl 3-(8-acetyl-2-oxo-1,8-diazaspiro[4.5]decan-3-yl)-2-((tert butoxycarbonyl) amino) propanoate (1) (4 g, 10.55 mmol) was separated by SFC separation to afford methyl (R)-3-((R)-8-acetyl-2-oxo-1,8-diazaspiro[4.5]decan-3-yl)-2-((tert-butoxycarbonyl)amino)propanoate (2) (Peak-1) and methyl (S)-3-((S)-8-acetyl-2-oxo-1,8-diazaspiro[4.5]decan-3-yl)-2-((tert-butoxycarbonyl)amino)propanoate (2) (Peak-2)

### Methyl (S)-3-((S)-8-acetyl-2-oxo-1,8-diazaspiro[4.5]decan-3-yl)-2-aminopropanoate hydrochloride (Int-7 Peak-2)

To the stirred solution of methyl 3-(8-acetyl-2-oxo-1,8-diazaspiro[4.5]decan-3-yl)-2-((tert-butoxycarbonyl)amino)propanoate (2 Peak-2) (2 g, 5.037 mmol) in DCM (5 mL), added dioxane HCl (5 mL). at 0° C. and stirred at room temperature for 2 h. The progress of the reaction was monitored by TLC and LCMS After 2 h, reaction mixture was evaporated under reduced pressure. The crude residue was purified by trituration with n-pentane afforded methyl (S)-3-((S)-8-acetyl-2-oxo-1,8-diazaspiro[4.5]decan-3-yl)-2-aminopropanoate hydrochloride (Int-7 Peak-2). TLC system: 10% methanol in dichloromethane Rf: 0.1 LCMS (ESI): m/z 298.4 [M+H]⁺

### Methyl (S)-3-((S)-8-acetyl-2-oxo-1,8-diazaspiro[4.5]decan-3-yl)-2-((S)-3-cyclohexyl-2-(1H-indole-2-carboxamido)propanamido)propanoate (3)

To a stirred solution of (R)-3-cyclohexyl-2-(1H-indole-2-carboxamido) propanoic acid (Amine fragment-6) (480 mg, 1.52 mmol) in DMF (5 mL) at 0° C. was added EDC.HCl (430 mg, 2.2 mmol), HOBT (309 mg, 2.2 mmol), DIPEA (0.814 ml, 4.5 mmol) and methyl (S)-3-((R)-8-acetyl-2-oxo-1,8-diazaspiro[4.5]decan-3-yl)-2-aminopropanoate hydrochloride (Int-7 Peak-2) (509 mg, 1.52 mmol) simultaneously and stirred at room temperature for 16 h. The progress of the reaction was monitored by TLC and LCMS. After 16 h, reaction mixture was quenched with ice water (50 mL), extracted with ethyl acetate (2×15 mL), the combined organic layer was dried over sodium sulfate and evaporated under reduced pressure. The crude residue was purified by silica gel column by eluting with 10% methanol in dichloromethane to afford methyl (S)-3-((S)-8-acetyl-2-oxo-1,8-diazaspiro[4.5]decan-3-yl)-2-((S)-3-cyclohexyl-2-(1H-indole-2-carboxamido)propanamido) propanoate (3). TLC system: 10% methanol in dichloromethane Rf: 0.5 LCMS (ESI): m/z 594.3 [M+H]⁺

### (S)-3-((S)-8-acetyl-2-oxo-1,8-diazaspiro[4.5]decan-3-yl)-2-((S)-3-cyclohexyl-2-(1H-indole-2-carboxamido)propanamido)propanoic acid (4)

To a stirred solution of methyl (S)-3-((S)-8-acetyl-2-oxo-1,8-diazaspiro[4.5]decan-3-yl)-2-((S)-3-cyclohexyl-2-(1H-indole-2-carboxamido)propanamido)propanoate (3) (700 mg, 1.1 mmol) in THF (4.5 mL), water (2.5 mL) was added lithium hydroxide (148 mg, 3.5 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×15 mL), dried over sodium sulfate, concentrated under reduced pressure to afford (S)-3-((S)-8-acetyl-2-oxo-1,8-diazaspiro[4.5]decan-3-yl)-2-((S)-3-cyclohexyl-2-(1H-indole-2-carboxamido)propanamido) propanoic acid (4). TLC system: 10% methanol in dichloromethane Rf: 0.2 LCMS (ESI): m/z 580.3 [M+H]⁺

### N—((S)-1-(((S)-1-((S)-8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-1H-indole-2-carboxamide (6)

To a stirred solution of (S)-3-((S)-8-acetyl-2-oxo-1,8-diazaspiro[4.5]decan-3-yl)-2-((S)-3-cyclohexyl-2-(1H-indole-2-carboxamido)propanamido)propanoic acid (4) (560 mg, 0.96 mmol) THF (5 mL) added HATU (550 mg, 1.44 mmol), DIPEA (0.54 mL, 2.8 mmol) and 1-(cyanomethyl)tetra hydro-1H-thiophen-1-iumbromide (5) (298 mg, 1.44 mmol) at 0° C. simultaneously and stirred at room temperature for 2 h. Reaction mixture was diluted with ice water (15 mL), extracted with dichloromethane (2×20 mL), dried over sodium sulfate and evaporated under reduced pressure. The crude residue was purified by combi-flash NP, compound eluted at 5% methanol in dichloromethane to N—((S)-1-(((S)-1-((S)-8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-1H-indole-2-carboxamide (6). TLC system: 10% Methanol in dichloromethane Rf: 0.5 LCMS (ESI): m/z 689.6 [M+H]⁺

### N—((S)-1-(((S)-1-((S)-8-acetyl-2-oxo-1,8-diazaspiro[4.5]decan-3-yl)-4-amino-3,4-dioxobutan-2-yl)amino)-3-cyclohexyl-1-oxopropan-2-yl)-1H-indole-2-carboxamide (Compound C125)

To a stirred solution of N—((S)-1-(((S)-1-((S)-8-acetyl-2-oxo-1,8-diazaspiro[4.5]decan-3-yl)-4-cyano-3-oxo-4-(tetrahydro-1l4-thiophen-1-ylidene)butan-2-yl)amino)-3-cyclohexyl-1-oxopropan-2-yl)-1H-indole-2-carboxamide (6) (200 mg, 0.29 mmol) in methanol (2 mL) was added m-CPBA (124 mg, 0.72 mmol) at 0° C. and the reaction mixture stirred for 2 h at 0° C. and added aq ammonia (1 mL) and stirred at RT for 2 h. The progress of the reaction was monitored by TLC and LCMS. Reaction mixture was quenched with sat. NaHCO₃ solution (15 mL) and extracted with DCM (2×15 mL). Organic layer was washed with brine solution (15 mL), dried over Na₂SO₄ and concentrated to get crude compound. The crude compound was purified by prep HPLC to afford N—((S)-1-(((S)-1-((S)-8-acetyl-2-oxo-1,8-diazaspiro[4.5]decan-3-yl)-4-amino-3,4-dioxobutan-2-yl)amino)-3-cyclohexyl-1-oxopropan-2-yl)-1H-indole-2-carboxamide (9) (Compound C125). TLC system: 10% Methanol in dichloromethane Rf: 0.3 LCMS (ESI): m/z 607.3 [M+H]⁺

### Example 102: Synthesis of Compound C142

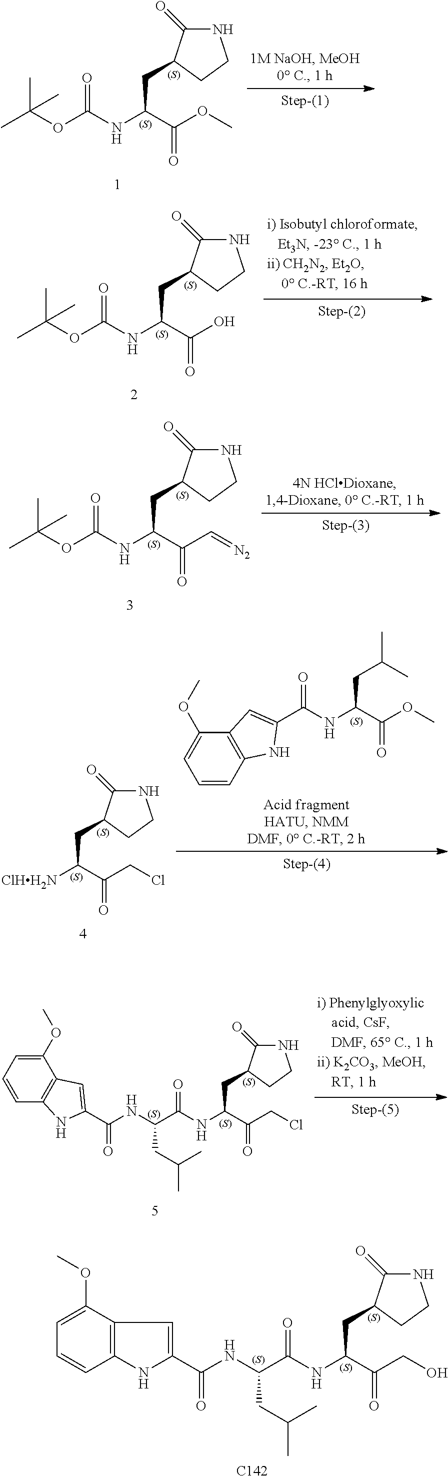

### (S)-2-((tert-Butoxycarbonyl)amino)-3-((S)-2-oxopyrrolidin-3-yl)propanoic acid (2)

A solution of methyl (S)-2-((tert-butoxycarbonyl)amino)-3-((S)-2-oxopyrrolidin-3-yl)propanoate (1) (1 g, 3.496 mmol) in methanol (10 mL) cooled to 0° C. then NaOH (500 mg in 5 mL water) solution was added. The resulting solution was stirred at 0° C. for 1 hour then removed the excess of methanol in vacuo. The residue was acidified to pH 3 and extracted with dichloromethane (2×50 ml), and the combined organics washed with brine, dried over MgSO4, concentrated under reduced pressure to afford (S)-2-((tert-Butoxycarbonyl)amino)-3-((S)-2-oxopyrrolidin-3-yl)propanoic acid (2). TLC system: 50% Ethyl acetate in Pet ether R_{f}: 0.2

### tert-Butyl ((S)-4-diazo-3-oxo-1-((S)-2-oxopyrrolidin-3-yl)butan-2-yl)carbamate (3)

A solution of (S)-2-((tert-Butoxycarbonyl)amino)-3-((S)-2-oxopyrrolidin-3-yl)propanoic acid (2) (3 g, 11.0 29 mmol) in THF (30 mL) was placed under an atmosphere of N2 and cooled to −23° C. The resulting clear colorless solution was successively treated with triethylamine (3 mL, 22.059 mmol) followed by isobutylchloroformate (1.8 mL, 13.235 mmol). The reaction mixture was slowly treated with a solution of diazomethane (˜50 mL, −25 mmol) in diethyl ether. The resulting yellow clear solution was gradually warmed to RT and stirred for 1 h. Progress of the reaction was monitored by TLC and LCMS. After that the reaction mixture was quenched with sat. NaHCO₃ solution (50 mL), and extracted with ethyl acetate (100 mL), washed once with water (50 mL), once with brine (50 mL), dried over MgSO4, filtered, and concentrated to give a. This material was purified by normal phase column chromatography to afford tert-Butyl ((S)-4-diazo-3-oxo-1-((S)-2-oxopyrrolidin-3-yl)butan-2-yl)carbamate (3). TLC system: 10% MeOH in DCM R_{f}: 0.5 LCMS (ESI): m/z 319.19 [M+Na]⁺

### (S)-3-((S)-2-amino-4-chloro-3-oxobutyl)pyrrolidin-2-one hydrochloride (4)

A solution of tert-butyl ((1S)-3-diazo-2-oxo-1-{[(3S)-2-oxopyrrolidin-3-yl]methyl}propyl) carbamate (3) (1.5 g, 5.06 mmol) in 1,4-dioxane (15 mL) was placed under an atmosphere of N2 and cooled to 0° C. This clear pale-yellow solution was drop-wise treated with a solution of 4M hydrochloric acid in 1,4-dioxane (15 mL). Upon complete addition, the reaction was warmed to RT over 1 h with the formation of white precipitate. Excess of 1,4-dioxane was evaporated under vacuum and the solid was triturated with diethyl ether to afford (S)-3-((S)-2-amino-4-chloro-3-oxobutyl)pyrrolidin-2-one hydrochloride (4). TLC system: 10% MeOH in DCM R_{f}: 0.1 LCMS (ESI): m/z 205.13 [M+H]⁺

### N—((S)-1-(((S)-4-chloro-3-oxo-1-((S)-2-oxopyrrolidin-3-yl)butan-2-yl)amino)-4-methyl-1-oxopentan-2-yl)-4-methoxy-1H-indole-2-carboxamide (5)

A solution of (S)-3-((S)-2-amino-4-chloro-3-oxobutyl)pyrrolidin-2-one hydrochloride (4) (500 mg, 2.083 mmol) and 4-methoxy-1H-indole-2-carboxylic acid (660 mg, 2.083 mmol) and in DMF (10 mL) was placed under an atmosphere of N2 and cooled to 0° C. This pale-yellow solution was successively treated with HATU (1.18 g, 3.124 mmol) and N-methylmorpholine (0.45 mL, 4.166 mmol). After 1 h, the reaction was quenched with 1:1 ice/sat NaHCO³ (50 mL) and extracted three times with ethyl acetate (50 mL). The combined organics were washed once with brine (100 mL), dried over MgSO4, filtered, and concentrated to give a yellow syrup. This material was purified by normal phase chromatography to afford N—((S)-1-(((S)-4-chloro-3-oxo-1-((S)-2-oxopyrrolidin-3-yl)butan-2-yl)amino)-4-methyl-1-oxopentan-2-yl)-4-methoxy-1H-indole-2-carboxamide (5). TLC system: 10% MeOH in DCM Rf: 0.6 LCMS (ESI): m/z 491.52 [M+H]⁺

### N—((S)-1-(((S)-4-Hydroxy-3-oxo-1-((S)-2-oxopyrrolidin-3-yl)butan-2-yl)amino)-4-methyl-1-oxopentan-2-yl)-4-methoxy-1H-indole-2-carboxamide (Compound C142)

A solution of N—((S)-1-(((S)-4-chloro-3-oxo-1-((S)-2-oxopyrrolidin-3-yl)butan-2-yl)amino)-4-methyl-1-oxopentan-2-yl)-4-methoxy-1H-indole-2-carboxamide (5) (200 mg, 0.428 mmol) and benzoylformic acid (80 mg, 0.53 mmol) in DMF (4 mL) was placed under an atmosphere of N₂. This clear pale-yellow solution was treated with cesium fluoride (155 mg, 1.02 mmol) followed by heating to 65° C. After 4 hr, the now yellow suspension was cooled to RT, diluted with ethyl acetate (60 mL), washed three times water (30 mL), once with brine (30 mL), dried over MgSO4, filtered, and concentrated to give crude (3S)-3-({N-[(4-methoxy-1H-indol-2-yl)carbonyl]-L-leucyl}amino)-2-oxo-4-[(3S)-2-oxopyrrolidin-3-yl]butyl oxo(phenyl)acetate as a crude yellow foam. MS (ESI+) for C₃₂H₃₆N₄O₈ m/z 605.2 (M+H)⁺. This crude was taken in methanol (20 mL) was placed under an atmosphere of N2 and treated with potassium carbonate (7 mg, 0.04 mmol) with vigorous stirring. After 1 hr the volatiles were removed in vacuo (bath <30° C.) to give a crude. This material was purified by normal phase chromatography to afford N—((S)-1-(((S)-4-Hydroxy-3-oxo-1-((S)-2-oxopyrrolidin-3-yl)butan-2-yl)amino)-4-methyl-1-oxopentan-2-yl)-4-methoxy-1H-indole-2-carboxamide (Compound C142) TLC system: 10% MeOH in DCM R_{f}: 0.55 LCMS (ESI): m/z 473.2 [M+H]⁺

### Example 103: Synthesis of Compound C128

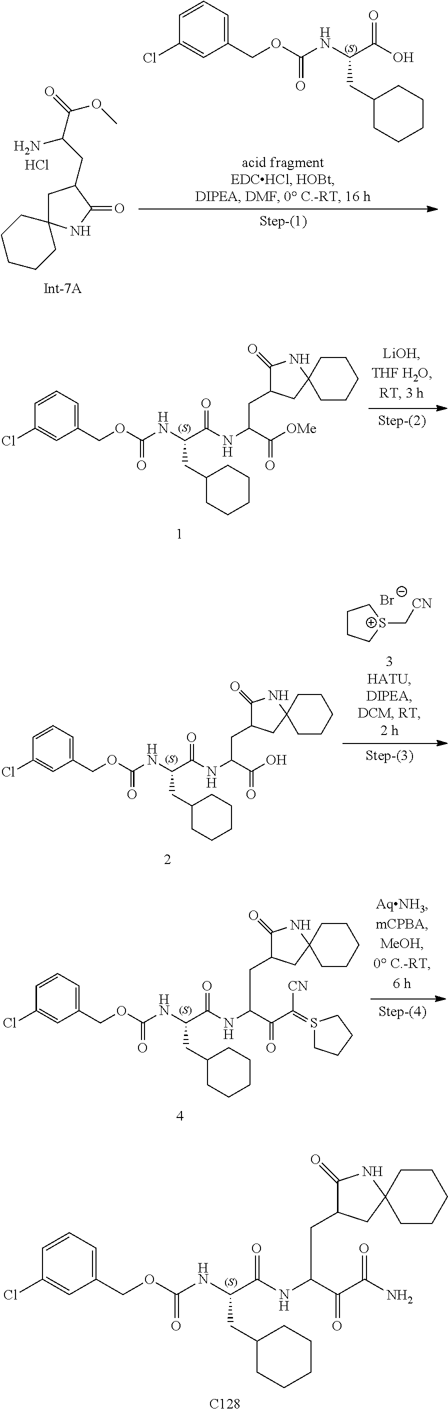

### Methyl 2-((S)-2-((((3-chlorobenzyl)oxy)carbonyl)amino)-3-cyclohexylpropanamido)-3-(2-oxo-1-azaspiro[4.5]decan-3-yl)propanoate (1)

To a stirred solution (S)-2-((((3-chlorobenzyl)oxy)carbonyl)amino)-3-cyclohexylpropanoic acid (Acid fragment) (1 g, 2.949 mmol) in DMF (20 mL) at 0° C. was added EDC.HCl (842 mg, 4.41 mmol), HOBT (595 mg, 4.41 mmol), DIPEA (1.52 mL, 8.823 mmol) and methyl 2-amino-3-(2-oxo-1-azaspiro[4.5]decan-3-yl)propanoate hydrochloride (Int-7A) (890 mg, 3.529 mmol) simultaneously and stirred at room temperature for 16 h. The progress of the reaction was monitored by TLC and LCMS. After 16 h, reaction mixture was quenched with ice water (50 mL), extracted with ethyl acetate (2×100 mL), the combined organic layer was dried over sodium sulfate and evaporated under reduced pressure. The crude residue was purified by silica gel column by eluting with 5% ethyl Methanol in DCM to afford Methyl2-((S)-2-((((3-chlorobenzyl)oxy)carbonyl)amino)-3-cyclohexylpropanamido)-3-(2-oxo-1-azaspiro[4.5]decan-3-yl)propanoate (1). TLC system: 5% Methanol in DCM R_{f}: 0.65 LCMS (ESI): m/z 577.63 (M+H)⁺

### 2-((S)-2-((((3-Chlorobenzyl)oxy)carbonyl)amino)-3-cyclohexylpropanamido)-3-(2-oxo-1-azaspiro[4.5]decan-3-yl)propanoic acid (2)

To a stirred solution of methyl methyl 2-((S)-2-((((3-chlorobenzyl)oxy)carbonyl)amino)-3-cyclohexylpropanamido)-3-(2-oxo-1-azaspiro[4.5]decan-3-yl)propanoate (1) (600 mg, 1.043 mmol) in THF (7 mL), water (3 mL) was added lithium hydroxide (100 mg, 4.173 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×20 mL), dried over sodium sulfate, concentrated under reduced pressure to afford 2-((S)-2-((((3-chlorobenzyl)oxy)carbonyl)amino)-3-cyclohexylpropanamido)-3-(2-oxo-1-azaspiro[4.5]decan-3-yl)propanoic acid (2). TLC system: 10% MeOH in DCM Rf: 0.2 LCMS (ESI): m/z 562.63 [M+H]⁺

### 3-Chlorobenzyl ((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (4)

To a stirred solution of 2-((S)-2-((((3-chlorobenzyl)oxy)carbonyl)amino)-3-cyclohexylpropanamido)-3-(2-oxo-1-azaspiro[4.5]decan-3-yl)propanoic acid (2) (450 mg, 0.802 mmol) in DCM (8 mL) added HATU (259 mg, 1.203 mmol), DIPEA (0.41 mL, 2.406 mmol) and 1-(cyanomethyl)tetrahydro-1H-thiophen-1-ium (3) (259 mg, 1.203 mmol) at 0° C. simultaneously and stirred at room temperature for 2 h. Reaction mixture was diluted with ice water (50 mL), extracted with EtoAc (2×20 mL), dried over sodium sulfate and evaporated under reduced pressure. The crude residue was purified by combi-flash NP, compound eluted at 5% methanol in dichloromethane to afford 3-chlorobenzyl ((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (4). TLC system: 10% Methanol in dichloromethane Rf: 0.4 LCMS (ESI): m/z 671.32 [M+H]⁺

### 3-Chlorobenzyl ((2S)-1-((4-amino-3,4-dioxo-1-(2-oxo-1-azaspiro[4.5]decan-3-yl)butan-2-yl)amino)-3-cyclohexyl-1-oxopropan-2-yl)carbamate (Compound C128)

To a stirred solution 3-chlorobenzyl ((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (4) (200 mg, 0.298 mmol) in methanol (3 mL) was added mCPBA (128 mg, 0.745 mmol) at 0° C. and the reaction mixture stirred for 2 h at 0° C. and added aq ammonia (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep-HPLC to afford 3-chlorobenzyl ((2S)-1-((4-amino-3,4-dioxo-1-(2-oxo-1-azaspiro[4.5]decan-3-yl)butan-2-yl)amino)-3-cyclohexyl-1-oxopropan-2-yl)carbamate (Compound C128). TLC system: 10% Methanol in dichloromethane Rf: 0.3 LCMS (ESI): m/z 589.2 [M+H]⁺

### Example 104: Synthesis of Compound C129

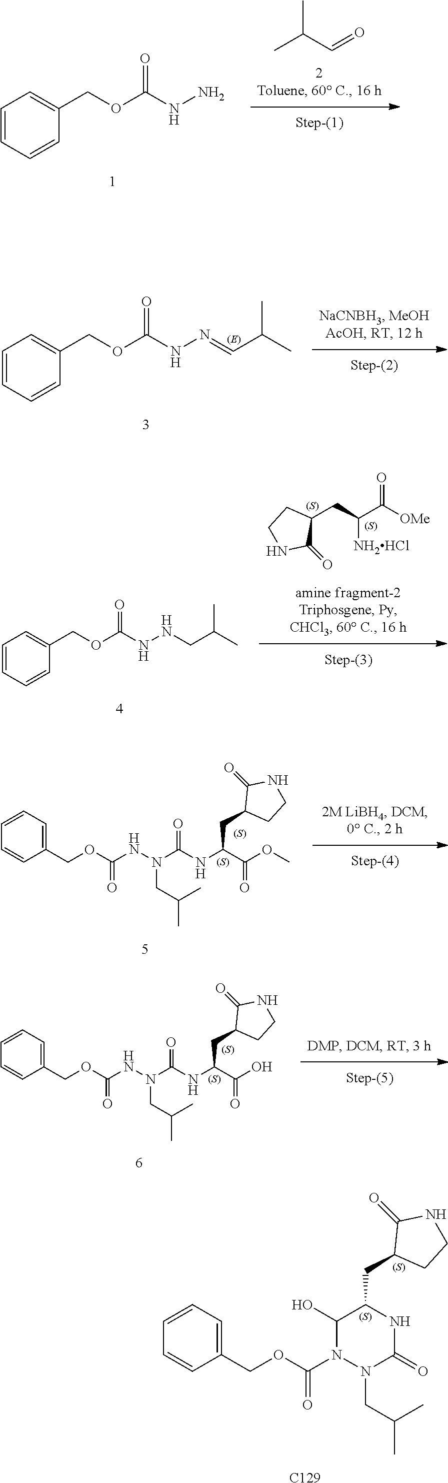

### Benzyl (E)-2-(2-methylpropylidene) hydrazine-1-carboxylate (3)

To a stirred solution of benzyl hydrazine carboxylate (1) (5 g, 30.12 mmol) in toluene (50 mL) was added isobutyraldehyde (2.7 mL, 30.12 mmol) stirred at 60° C. for 16 h. The progress of the reaction was monitored by TLC. Reaction mixture was directly concentrated under reduced pressure to obtained crude compound, the resulting crude was triturated with diethyl ether to afford benzyl (E)-2-(2-methylpropylidene) hydrazine-1-carboxylate (3). TLC system: 20% Ethyl acetate in hexane Rf: 0.7 LCMS (ESI): m/z 221.29 [M+H]⁺

### Benzyl 2-isobutylhydrazine-1-carboxylate (4)

To a stirred solution of benzyl (E)-2-(2-methylpropylidene)hydrazine-1-carboxylate (3) (3 g, 13.574 mmol) in methanol (20 mL) was added acetic acid (0.5 mL) followed by sodium cyano borohydride (1.7 g, 27.149 mmol) at 0° C. and stirred at room temperature for 16 h. The progress of the reaction was monitored by TLC and LCMS. Reaction mixture was quenched with ice cold water and extracted with ethyl acetate (2×50 mL), organic layer was dried over sodium sulfate, filtered and evaporated under reduced pressure. The crude residue was purified by combi-flash chromatography and compound eluted at 10% ethyl acetate in pet ether to afford benzyl 2-iso butylhydrazine-1-carboxylate (4). TLC system: 20% Ethyl acetate in hexane Rf: 0.3 LCMS (ESI): m/z 223.26 [M+H]⁺

### Benzyl 2-isobutyl-2-(((S)-1-methoxy-1-oxo-3-((S)-2-oxopyrrolidin-3-yl) propan-2-yl)carbamoyl)hydrazine-1-carboxylate (5)

To a stirred solution of benzyl 2-isobutylhydrazine-1-carboxylate (4) (1 g, 4.504 mmol) chloroform (15 mL) added pyridine (1 mL, 1 vol), and methyl (S)-2-amino-3-((S)-2-oxo pyrrolidin-3-yl) propanoate (amine fragment-2) (1 g, 5.405 mmol) added triphosgene (666 mg, 2.252 mmol,) simultaneously at 0° C. and stirred at 60° C. for 16 h. The progress of the reaction was monitored by TLC and LCMS. The reaction mixture was diluted with ice water (50 mL), extracted with dichloromethane (2×50 mL). The combined organic layer was dried over sodium sulfate, filtered and evaporated under reduced pressure. The crude residue was purified by combi-flash chromatography and compound eluted at 8% methanol in dichloromethane to afford benzyl2-isobutyl-2-(((S)-1-methoxy-1-oxo-3-((S)-2-oxopyrrolidin-3-yl) propan-2-yl) carbamoyl) hydrazine-1-carboxylate (5). TLC system: 10% Methanol in dichloromethane Rf: 0.5 LCMS (ESI): m/z 435.39 [M+H]⁺

### Benzyl 2-(((S)-1-hydroxy-3-((S)-2-oxopyrrolidin-3-yl) propan-2-yl) carbamoyl)-2-isobutylhydrazine-1-carboxylate (6)

To a stirred solution of benzyl 2-isobutyl-2-(((S)-1-methoxy-1-oxo-3-((S)-2-oxo pyrrolidin-3-yl)propan-2-yl)carbamoyl)hydrazine-1-carboxylate (5) (1 g, 2.304 mmol) in DCM (10 mL) was added 2M LiBH₄ in THF (2.3 mL, 4.608 mmol) at 0° C. and the reaction mixture stirred for 2 h at 0° C. The progress of the reaction was monitored by TLC and LCMS. Reaction mixture was quenched with saturated ammonium chloride solution (20 mL) and extracted with DCM (2×30 mL). Organic layer was washed with brine solution (30 mL), dried over Na₂SO₄ and concentrated to afford benzyl 2-(((S)-1-hydroxy-3-((S)-2-oxopyrrolidin-3-yl) propan-2-yl) carbamoyl)-2-isobutylhydrazine-1-carboxylate (6). TLC system: 10% Methanol in dichloromethane Rf: 0.2 LCMS (ESI): m/z 407.69 [M+H]⁺

### Benzyl (5S)-6-hydroxy-2-isobutyl-3-oxo-5-(((S)-2-oxopyrrolidin-3-yl) methyl)-1,2,4-triazinane-1-carboxylate (Compound C129)

To a stirred solution of benzyl 2-(((S)-1-hydroxy-3-((S)-2-oxopyrrolidin-3-yl)propan-2-yl)carbamoyl)-2-isobutylhydrazine-1-carboxylate (6) (150 mg, 0.369 mmol) was dissolved in dichloromethane (10 mL) was added Dess-Martin periodinane (469 mg, 1.107 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford benzyl (5S)-6-hydroxy-2-isobutyl-3-oxo-5-(((S)-2-oxopyrrolidin-3-yl) methyl)-1,2,4-triazinane-1-carboxylate (Compound C129). TLC system: 10% Methanol in dichloromethane Rf: 0.5 LCMS (ESI): m/z 405.1 (M+H)⁺

### Example 105: Synthesis of Compounds C132 and C131

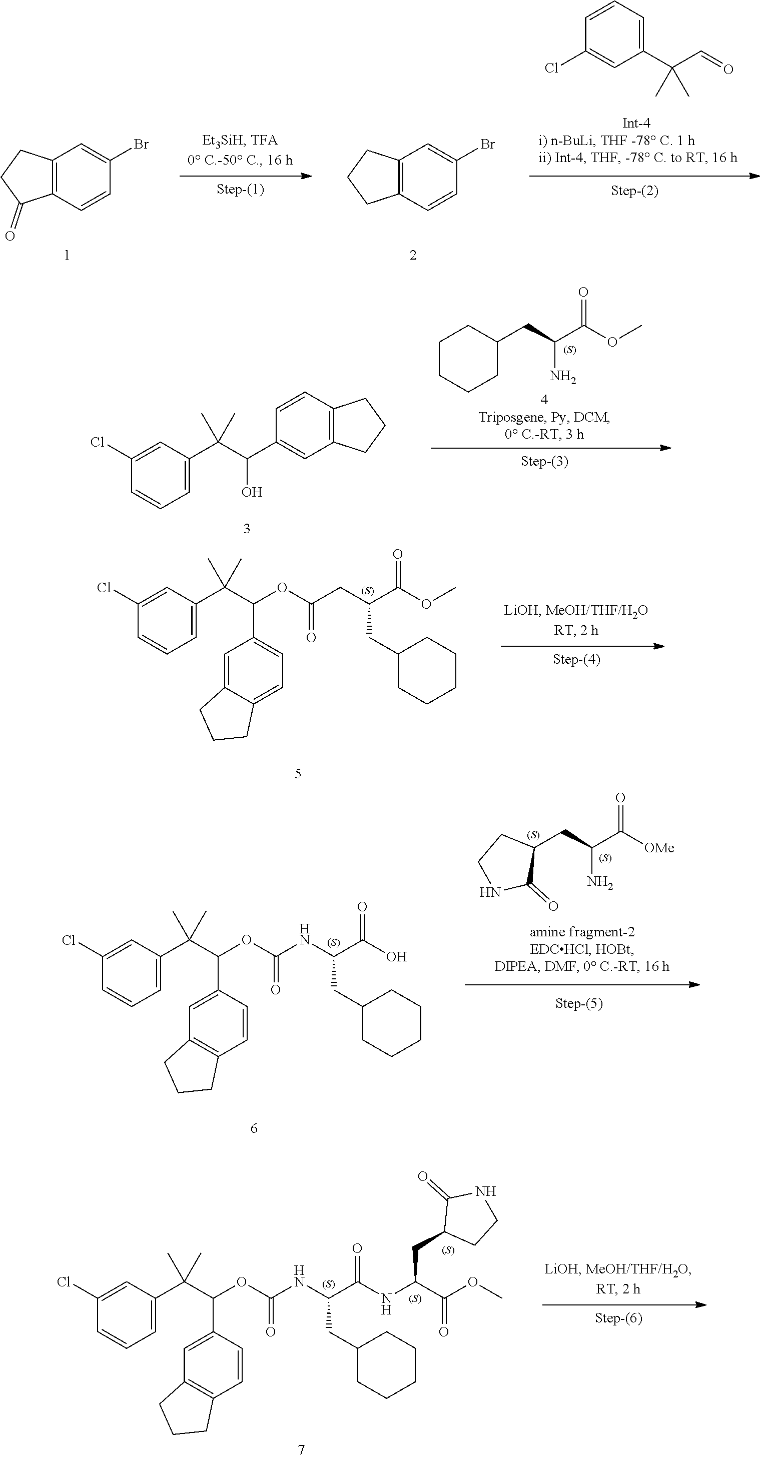

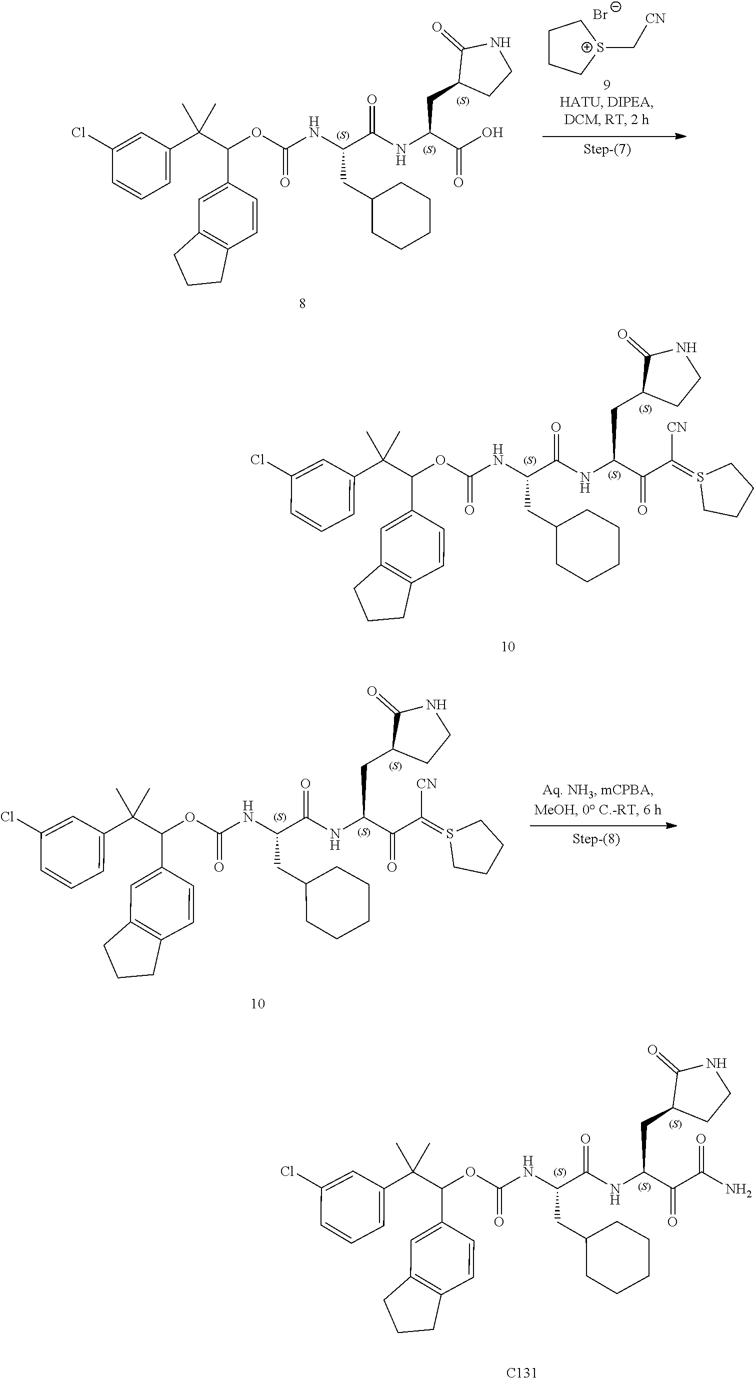

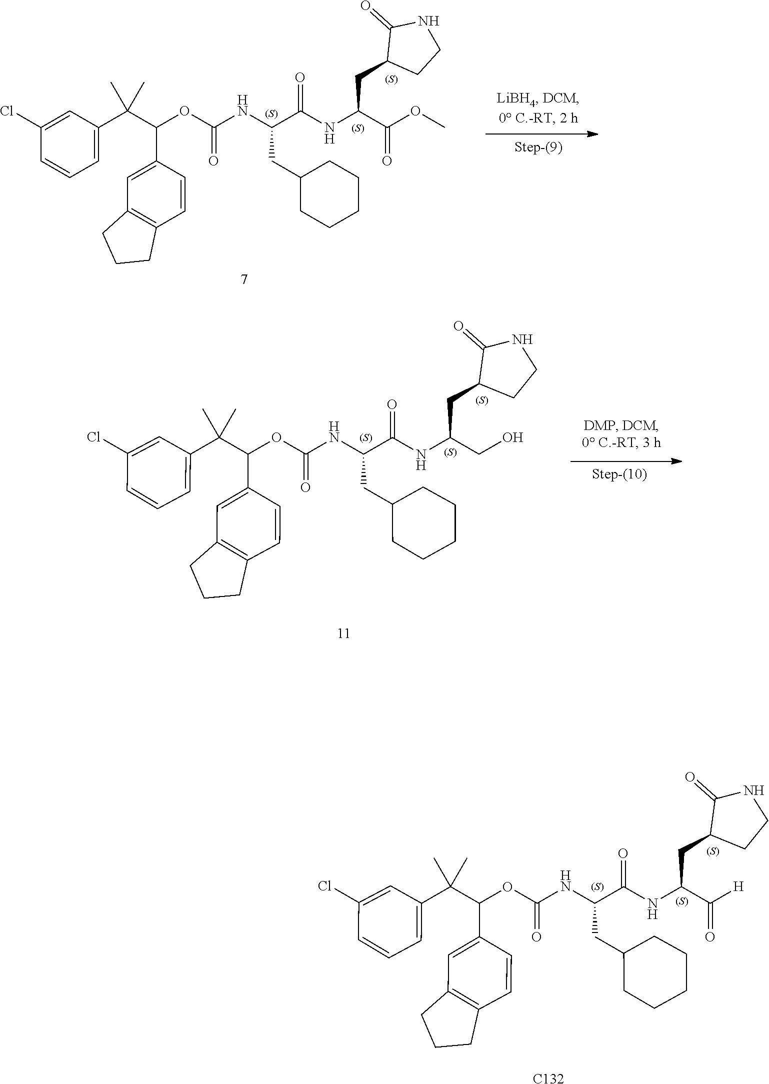

### 2-(3-Chlorophenyl)-2-methyl-1-(naphthalen-2-yl) propan-1-ol (2)

To a stirred solution of 5-bromo-2,3-dihydro-1H-inden-1-one (10 g, 47.846 mmol) in trifluoro acetic acid (50 mL) was slowly added triethyl silane (76 mL, 478.6 mmol) at 0° C. then the resulting reaction mixture was stirred at 50° C. for 16 h. The progress of the reaction was monitored by TLC. Reaction mixture was poured into ice water and extracted with ethyl acetate (2×100 mL). Combined organic layers were neutralized with aq. sodium bicarbonate solution (2×200 mL) and washed with water (200 mL), dried over sodium sulfate, concentrated under reduced pressure to afford crude residue, crude residue was purified by normal phase chromatography to afford 5-bromo-2,3-dihydro-1H-indene (2). TLC system: Pet. ether R_{f}: 0.8 GCMS (ESI): m/z 196.7 [M+H]⁺

### 2-(3-Chlorophenyl)-1-(2,3-dihydro-1H-inden-5-yl)-2-methylpropan-1-ol (3)

A solution of 5-bromo-2,3-dihydro-1H-indene (2) (2.5 g, 12.820 mmol), in THF (25 mL) was cooled to −78° C., then n-butyl lithium 2.0 M in THF (7.69 mL, 15.384 mmol) was added drop-wise into the above mixture. The mixture was stirred at −78° C. for 1 h. After that a solution of 2-(3-chlorophenyl)-2-methylpropanal (Int-4) (3.5 g, 19.23 mmol) in THF (5 mL) was added, and stirred at room temperature for 16 h. Progress of the reaction was monitored by TLC and LCMS. The mixture was quenched with aq. NH₄Cl solution and extracted with ethyl acetate (2×100 mL) and washed with water (100 mL), dried over sodium sulfate, concentrated under reduced pressure and purified by normal phase chromatography to afford 2-(3-chlorophenyl)-1-(2,3-dihydro-1H-inden-5-yl)-2-methylpropan-1-ol (3). TLC system: 5% Ethyl acetate in pet. ether R_{f}: 0.4 LCMS (ESI): m/z 284.76 [M−OH]⁺

### Methyl (2S)-2-(((2-(3-chlorophenyl)-1-(2,3-dihydro-1H-inden-5-yl)-2-methylpropoxy)carbonyl)amino)-3-cyclohexylpropanoate (5)

To a stirred solution of 2-(3-chlorophenyl)-1-(2,3-dihydro-1H-inden-5-yl)-2-methylpropan-1-ol (3) (4 g, 13.33 mmol), methyl (S)-2-amino-3-cyclohexylpropanoate hydrochloride (4) (3.53 g, 15.99 mmol) in DCM (40 mL) was added pyridine (4 mL, 1 vol) followed by triphosgene (1.97 g, 6.66 mmol) at 0° C. and stirred at room temperature for 3 h. The progress of the reaction was monitored by TLC and LCMS. Reaction mixture was quenched with aq. NaHCO₃ solution (100 mL), extracted with DCM (2×50 mL). The combined organic layer was dried over sodium sulfate, filtered and evaporated under reduced pressure. The crude residue was purified by normal phase chromatography to afford methyl (2S)-2-(((2-(3-chlorophenyl)-1-(2,3-dihydro-1H-inden-5-yl)-2-methylpropoxy)carbonyl)amino)-3-cyclohexylpropanoate (5). TLC system: 10% Ethyl acetate in Pet ether R_{f}: 0.4 LCMS (ESI): m/z 534.37 [M+Na]⁺

### (2S)-2-(((2-(3-Chlorophenyl)-1-(2,3-dihydro-1H-inden-5-yl)-2-methylpropoxy)carbonyl)amino)-3-cyclohexylpropanoic acid (6)

To a stirred solution of methyl (2S)-2-(((2-(3-chlorophenyl)-1-(2,3-dihydro-1H-inden-5-yl)-2-methylpropoxy)carbonyl)amino)-3-cyclohexylpropanoate (4) (3.5 g, 6.849 mmol) in MeOH/THF (20 mL), water (10 mL) was added lithium hydroxide (0.86 g, 20.549 mmol) at RT and stirred at room temperature for 16 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), The combined organic layers were washed with brine solution (20 mL), dried over Na₂SO₄ and concentrated to afford (2S)-2-(((2-(3-chlorophenyl)-1-(2,3-dihydro-1H-inden-5-yl)-2-methylpropoxy)carbonyl) amino)-3-cyclohexylpropanoic acid (5). TLC system: 10% MeOH in DCM R_{f}: 0.1 LCMS (ESI): m/z 520.29 [M+Na]⁺

### Methyl (2S)-2-((2S)-2-(((2-(3-chlorophenyl)-1-(2,3-dihydro-1H-inden-5-yl)-2-methylpropoxy)carbonyl)amino)-3-cyclohexylpropanamido)-3-((S)-2-oxopyrrolidin-3-yl)propanoate (6)

To a stirred solution of (2S)-2-(((2-(3-chlorophenyl)-1-(2,3-dihydro-1H-inden-5-yl)-2-methylpropoxy)carbonyl) amino)-3-cyclohexylpropanoic acid (6) (2.5 g, 5.03 mmol) in DMF (50 mL) was added EDC.HCl (1.44 g, 7.545 mmol), HOBt (1.01 g, 7.545 mmol), and DIPEA (2.72 mL, 15.09 mmol) at 0° C., then methyl 2-amino-2-methyl-3-(2-oxopyrrolidin-3-yl)propanoate hydrochloride (amine fragment-2) (1.34 g, 6.036 mmol) at 0° C. and stirred at room temperature for 16 h. Reaction mixture was diluted with ice water (50 mL), extracted with ethyl acetate (2×50 mL), dried over sodium sulfate and evaporated under reduced pressure. The crude was purified by flash chromatography to afford methyl(2S)-2-((2S)-2-(((2-(3-chlorophenyl)-1-(2,3-dihydro-1H-inden-5-yl)-2-methylpropoxy) carbonyl)amino)-3-cyclohexylpropanamido)-3-((S)-2-oxopyrrolidin-3-yl)propanoate (7). TLC system: 5% MeOH in DCM R_{f}: 0.4 LCMS (ESI): m/z 666.47 [M+H]⁺

### (2S)-2-((2S)-2-(((2-(3-chlorophenyl)-1-(2,3-dihydro-1H-inden-5-yl)-2-methylpropoxy)carbonyl)amino)-3-cyclohexylpropanamido)-3-((S)-2-oxopyrrolidin-3-yl)propanoic acid (8)

To a stirred solution of methyl(2S)-2-((2S)-2-(((2-(3-chlorophenyl)-1-(2,3-dihydro-1H-inden-5-yl)-2-methylpropoxy)carbonyl)amino)-3-cyclohexylpropanamido)-3-((S)-2-oxopyrrolidin-3-yl)propanoate (7) (850 mg, 1.278 mmol) in THF (4 mL), water (2 mL) was added lithium hydroxide (165 mg, 3.83 mmol) at 0° C. and stirred at room temperature for 2 h. The progress of the reaction was monitored by TLC and LCMS. Reaction mixture completely distilled under reduced pressure, crude compound acidified with aq. 1N HCl solution up to pH˜3 and extracted with dichloromethane (2×100 mL), dried over sodium sulfate, concentrated under reduced pressure to afford (2S)-2-((2S)-2-(((2-(3-chlorophenyl)-1-(2,3-dihydro-1H-inden-5-yl)-2-methylpropoxy)carbonyl)amino)-3-cyclohexylpropanamido)-3-((S)-2-oxopyrrolidin-3-yl)propanoic acid (8). 10% Methanol in dichloromethane R_{f}: 0.1 LCMS (ESI): m/z 652.53 [M+H]⁺

### 2-(3-Chlorophenyl)-1-(2,3-dihydro-1H-inden-5-yl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (10)

To a stirred solution of (2S)-2-((2S)-2-(((2-(3-chlorophenyl)-1-(2,3-dihydro-1H-inden-5-yl)-2-methylpropoxy)carbonyl)amino)-3-cyclohexylpropanamido)-3-((S)-2-oxopyrrolidin-3-yl)propanoic acid (8) (350 mg, 0.453 mmol) DMF (10 mL) added HATU (306 mg, 0.806 mmol), DIPEA (0.29 mL, 1.6128 mmol) and 1-(cyanomethyl)tetrahydro-1H-thiophen-1-iumbromide (9) (167 mg, 0.806 mmol) at 0° C. simultaneously and stirred at room temperature for 2 h. Reaction mixture was diluted with ice water (25 mL), extracted with 5% methanol in dichloromethane (2×30 mL), dried over sodium sulfate and evaporated under reduced pressure. The crude residue was purified by normal phase chromatography to afford 2-(3-chlorophenyl)-1-(2,3-dihydro-1H-inden-5-yl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (10). TLC system: 10% Methanol in dichloromethane R_{f}: 0.3 LCMS (ESI): m/z 761.63 [M+H]⁺

### 2-(3-Chlorophenyl)-1-(2,3-dihydro-1H-inden-5-yl)-2-methylpropyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C131)

To a stirred solution of 2-(3-chlorophenyl)-1-(2,3-dihydro-1H-inden-5-yl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (10) (150 mg, 0.197 mmol) in methanol (5 mL) was added m-CPBA (65%) (105 mg, 0.3947 mmol) at 0° C. and the reaction mixture stirred for 2 h at 0° C. and then added aq. ammonia (1.5 mL) and stirred at RT for 6 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford 2-(3-chlorophenyl)-1-(2,3-dihydro-1H-inden-5-yl)-2-methylpropyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C131). TLC system: 10% Methanol in DCM R_{f}: 0.3 LCMS (ESI): m/z 679.1 [M+H]⁺

### 2-(3-Chlorophenyl)-1-(2,3-dihydro-1H-inden-5-yl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (11)

To a stirred solution of methyl (2S)-2-((2S)-2-(((2-(3-chlorophenyl)-1-(2,3-dihydro-1H-inden-5-yl)-2-methylpropoxy)carbonyl)amino)-3-cyclohexylpropanamido)-3-((S)-2-oxo pyrrolidin-3-yl)propanoate (7) (500 mg, 0.7518 mmol) in DCM (6 mL) was added 2M LiBH₄ in THF (0.75 mL, 1.503 mmol) at 0° C. and the reaction mixture stirred for 2 h at 0° C. The progress of the reaction was monitored by TLC and LCMS. Reaction mixture was quenched with sat. ammonium chloride solution (30 mL) and extracted with DCM (2×30 mL). Organic layer was washed with brine solution (20 mL), dried over Na₂SO₄ and concentrated to afford 2-(3-chlorophenyl)-1-(2,3-dihydro-1H-inden-5-yl)-2-methylpropyl((S)-3-cyclohexyl-1-(((S)-1-hydroxy-3-((S)-2-oxopyrrolidin-3-yl)propan-2-yl)amino)-1-oxopropan-2-yl)carbamate (11). TLC system: 10% MeOH in DCM R_{f}: 0.4 LCMS (ESI): m/z 638.74 [M+H]⁺

### 2-(3-Chlorophenyl)-1-(2,3-dihydro-1H-inden-5-yl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (Compound C132)

To a stirred solution of 2-(3-chlorophenyl)-1-(2,3-dihydro-1H-inden-5-yl)-2-methylpropyl((S)-3-cyclohexyl-1-(((S)-1-hydroxy-3-((S)-2-oxopyrrolidin-3-yl)propan-2-yl)amino)-1-oxopropan-2-yl)carbamate (11) (200 mg, 0.3139 mmol) in ethyl acetate (5 mL) was added Dess-Martin periodinane (399 mg, 0.9419 mmol) at 0° C. and stirred at RT for 16 h. The progress of the reaction was monitored by TLC and LCMS. After complete consumption of the starting material by TLC and LCMS, the reaction mass was filtered through celite pad and the celite pad thoroughly was with ethyl acetate (30 mL). Then the organic layer was washed with 10% sodium thiosulfate solution (2×50 mL) followed by saturated sodium bicarbonate solution (2×50 mL), water (1×50 mL), brine (1×50 mL). Then the organic layer was dried over sodium sulfate and evaporated under vacuum. Then the crude compound was purified by Prep-HPLC purification to get 2-(3-chlorophenyl)-1-(2,3-dihydro-1H-inden-5-yl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (Compound C132). TLC system: 10% MeOH in DCM R_{f}: 0.45 LCMS (ESI): m/z 636.00 (M+H)⁺

### Example 106: Synthesis of Compounds C145 and C134

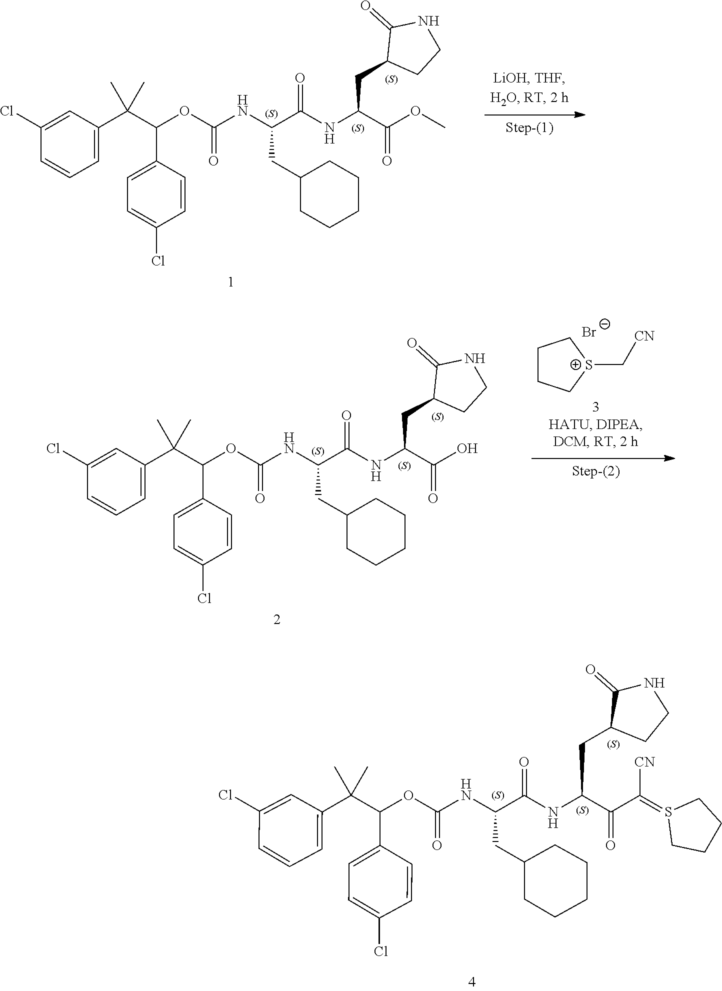

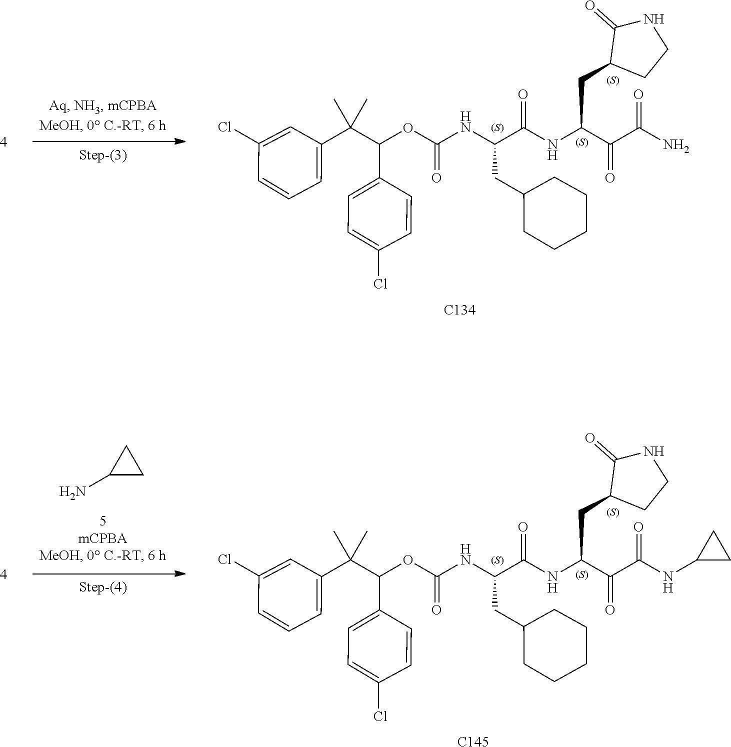

### (2S)-2-((2S)-2-(((2-(3-chlorophenyl)-1-(4-chlorophenyl)-2-methylpropoxy) carbonyl) amino)-3-cyclohexylpropanamido)-3-((S)-2-oxopyrrolidin-3-yl) propanoic acid (2)

To a stirred solution of methyl (2S)-2-((2S)-2-(((2-(3-chlorophenyl)-1-(4-chlorophenyl)-2-methylpropoxy)carbonyl)amino)-3-cyclohexylpropanamido)-3-((S)-2-oxopyrrolidin-3-yl) propanoate (1) (2.5 g, 3.793 mmol) in THF (10 mL), water (5 mL) was added lithium hydroxide (466 mg, 11.380 mmol) at RT and stirred at room temperature for 2 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate and concentrated under reduced pressure to afford (2S)-2-((2S)-2-(((2-(3-chlorophenyl)-1-(4-chlorophenyl)-2-methylpropoxy)carbonyl)amino)-3-cyclohexylpropanamido)-3-((S)-2-oxo pyrrolidin-3-yl)propanoic acid (2). TLC system: 15% Methanol in dichloromethane Rf: 0.2 LCMS (ESI): m/z 646.49 [M+H]⁺

### 2-(3-Chlorophenyl)-1-(4-chlorophenyl)-2-methylpropyl ((S)-1-(((S)-4-cyano-3-oxo-1-((S)-2-oxopyrrolidin-3-yl)-4-(tetrahydro-1l4-thiophen-1-ylidene) butan-2-yl) amino)-3-cyclohexyl-1-oxopropan-2-yl) carbamate (4)

To a stirred solution of (2S)-2-((2S)-2-(((2-(3-chlorophenyl)-1-(4-chlorophenyl)-2-methylpropoxy)carbonyl)amino)-3-cyclohexylpropanamido)-3-((S)-2-oxopyrrolidin-3-yl) propanoic acid (2) (500 mg, 0.775 mmol) DCM (10 mL) added HATU (441 mg, 1.16 mmol), DIPEA (0.4 mL, 2.32 mmol) and 1-(cyanomethyl)tetra hydro-1H-thiophen-1-iumbromide (3) (239 mg, 1.162 mmol) at 0° C. simultaneously at 0° C. and stirred at room temperature at RT for 2 h. Reaction mixture was quenched with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 2-(3-chlorophenyl)-1-(4-chloro phenyl)-2-methylpropyl((S)-1-(((S)-4-cyano-3-oxo-1-((S)-2-oxopyrrolidin-3-yl)-4-(tetrahydro-1l4-thiophen-1-ylidene) butan-2-yl)amino)-3-cyclohexyl-1-oxopropan-2-yl) carbamate (4). TLC system: 10% Methanol in dichloromethane Rf: 0.4 LCMS (ESI): m/z 755.47 [M+H]⁺

### 2-(3-Chlorophenyl)-1-(4-chlorophenyl)-2-methylpropyl ((S)-1-(((S)-4-amino-3,4-dioxo-1-((S)-2-oxopyrrolidin-3-yl) butan-2-yl) amino)-3-cyclohexyl-1-oxopropan-2-yl)carbamate (Compound C134)

To a stirred solution of 2-(3-chlorophenyl)-1-(4-chlorophenyl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (4) (200 mg, 0.265 mmol) in methanol (3 mL) was added mCPBA (91 mg, 0.530 mmol) at 0° C. and the reaction mixture stirred for 2 h at 0° C. and added aq ammonia (2 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over sodium sulfate and concentrated to get crude compound. The crude compound was purified by prep-HPLC to afford 2-(3-chlorophenyl)-1-(4-chlorophenyl)-2-methylpropyl ((S)-1-(((S)-4-amino-3,4-dioxo-1-((S)-2-oxopyrrolidin-3-yl)butan-2-yl)amino)-3-cyclohexyl-1-oxopropan-2-yl) carbamate (Compound C134). TLC system: 10% Methanol in dichloromethane Rf: 0.3 LCMS (ESI): m/z 673.1 [M+H]+

### 2-(3-Chlorophenyl)-1-(4-chlorophenyl)-2-methylpropyl ((S)-3-cyclohexyl-1-(((S)-4-(cyclopropylamino)-3,4-dioxo-1-((S)-2-oxopyrrolidin-3-yl) butan-2-yl) amino)-1-oxopropan-2-yl) carbamate (Compound C145)

To a stirred solution of 2-(3-chlorophenyl)-1-(4-chlorophenyl)-2-methylpropyl 2-(3-chloro phenyl)-1-(4-chlorophenyl)-2-methylpropyl((S)-1-(((S)-4-cyano-3-oxo-1-((S)-2-oxo pyrrolidin-3-yl)-4-(tetrahydro-1l4-thiophen-1-ylidene) butan-2-yl)amino)-3-cyclohexyl-1-oxo propan-2-yl) carbamate (4) (0.2 g, 0.265 mmol) in methanol (4 mL) was added m-CPBA (91 mg, 0.530 mmol) at 0° C. and the reaction mixture stirred for 2 h at 0° C. and added cyclopropyl amine (2.0 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford 2-(3-chlorophenyl)-1-(4-chlorophenyl)-2-methylpropyl((S)-3-cyclohexyl-1-(((S)-4-(cyclopropyl amino)-3,4-dioxo-1-((S)-2-oxopyrrolidin-3-yl) butan-2-yl) amino)-1-oxopropan-2-yl) carbamate (Compound C145). TLC system: 10% Methanol in dichloromethane Rf: 0.2 LCMS (ESI): m/z 713.2 [M+H]⁺

### Example 107: Synthesis of Compound C138

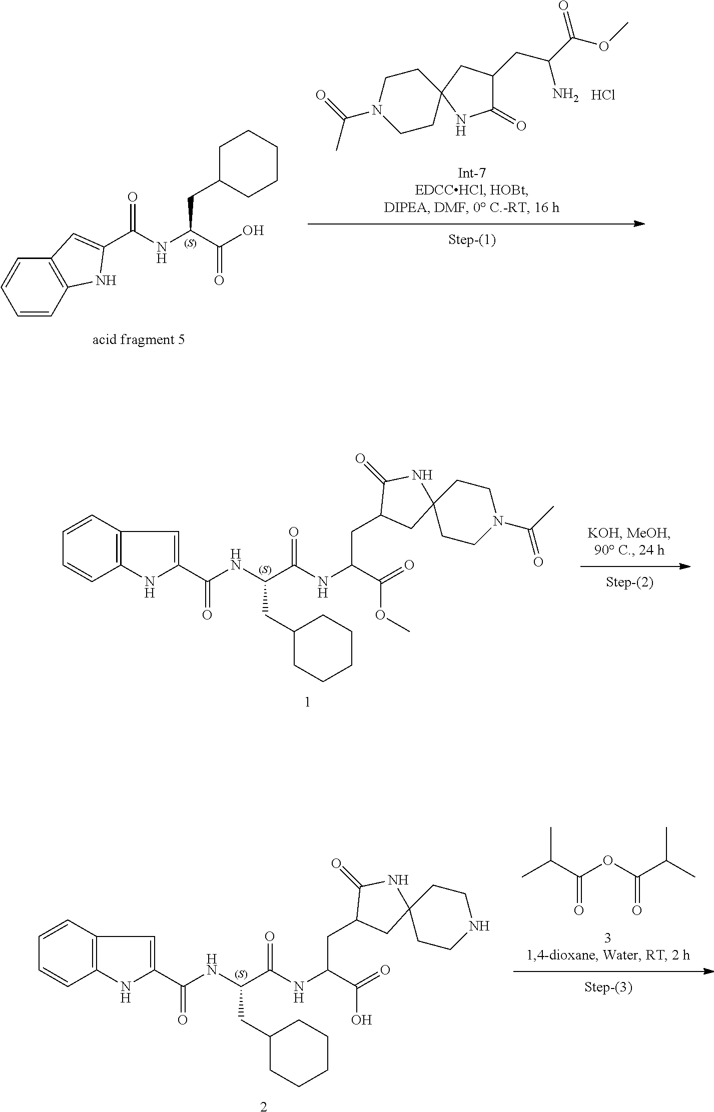

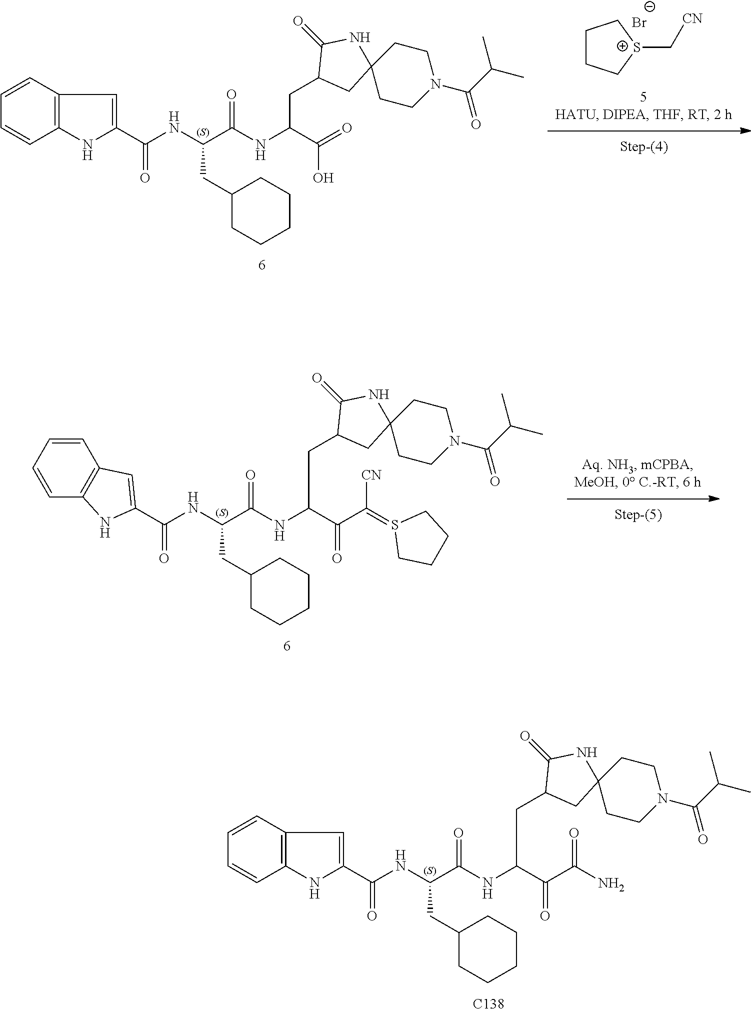

### Methyl (S)-3-((S)-8-acetyl-2-oxo-1,8-diazaspiro[4.5]decan-3-yl)-2-((S)-3-cyclohexyl-2-(1H-indole-2-carboxamido)propanamido)propanoate (1)

To a stirred solution of (R)-3-cyclohexyl-2-(1H-indole-2-carboxamido) propanoic acid (Acid fragment-5) (1 g, 3.1 mmol) in DMF (10 mL) at 0° C. was added EDC.HCl (912 mg, 4.7 mmol), HOBT (644 mg, 4.7 mmol), DIPEA (1.69 mL, 9.5 mmol) and methyl (S)-3-((R)-8-acetyl-2-oxo-1,8-diazaspiro[4.5]decan-3-yl)-2-aminopropanoate hydrochloride (Int-7) (1.16 g, 3.5 mmol) simultaneously and stirred at room temperature for 16 h. The progress of the reaction was monitored by TLC and LCMS. After 16 h, reaction mixture was quenched with ice water (50 mL), extracted with ethyl acetate (2×25 mL), the combined organic layer was dried over sodium sulfate and evaporated under reduced pressure. The crude residue was purified by silica gel column by eluting with 10% methanol in dichloromethane to afford methyl (S)-3-((S)-8-acetyl-2-oxo-1,8-diazaspiro[4.5]decan-3-yl)-2-((S)-3-cyclohexyl-2-(1H-indole-2-carboxamido)propanamido)propanoate (1) (1.6 g, 2.6 mmol, 89% yield) as pale green color solid. TLC system: 10% methanol in dichloromethane Rf: 0.5 LCMS (ESI): m/z 594.3 [M+H]⁺

### 2-((S)-3-Cyclohexyl-2-(1H-indole-2-carboxamido)propanamido)-3-(2-oxo-1,8-diazaspiro[4.5]decan-3-yl)propanoic acid (2)

To a stirred solution of methyl (S)-3-((S)-8-acetyl-2-oxo-1,8-diazaspiro[4.5]decan-3-yl)-2-((S)-3-cyclohexyl-2-(1H-indole-2-carboxamido)propanamido)propanoate (1) (500 mg, 0.84 mmol) in Methanol (10 mL) was added potassium hydroxide (280 mg, 5.05 mmol) at RT and stirred at room temperature for 24 h. The progress of the reaction was monitored by TLC and LCMS. Reaction mixture completely distilled under reduced pressure to afford 2-((S)-3-cyclohexyl-2-(1H-indole-2-carboxamido)propanamido)-3-(2-oxo-1,8-diazaspiro[4.5]decan-3-yl)propanoic acid (2) (450 mg, 0.83 mmol, 99.7% yield) as a yellow solid. TLC system: 15% methanol in dichloromethane Rf: 0.2 LCMS (ESI): m/z 538.6 [M+H]⁺

### 2-((S)-3-Cyclohexyl-2-(1H-indole-2-carboxamido)propanamido)-3-(8-isobutyryl-2-oxo-1,8-diazaspiro[4.5]decan-3-yl)propanoic acid (4)

To a stirred solution of 2-((S)-3-cyclohexyl-2-(1H-indole-2-carboxamido)propanamido)-3-(2-oxo-1,8-diazaspiro[4.5]decan-3-yl)propanoic acid (2) (450 mg, 0.83 cmmol) in Dioxane (4.5 mL) and water (2.2 mL) at 0° C. was added isobutyric anhydride (3) (0.65 mL, 4.18 mmol) at RT and stirred at room temperature for 2 h. The progress of the reaction was monitored by TLC and LCMS. Reaction mixture completely distilled under reduced pressure and acidify by 1N HCl extracted with dichloromethane (2×20 mL), dried over sodium sulfate and evaporated under reduced pressure. The crude residue was purified by combi-flash RP, compound eluted at 20% Acetonitrile in Water to afford 2-((S)-3-cyclohexyl-2-(1H-indole-2-carboxamido)propanamido)-3-(8-isobutyryl-2-oxo-1,8-diazaspiro[4.5]decan-3-yl)propanoic acid (4) (150 mg, 0.24 mmol, 29.4% yield) as a white solid. TLC system: 15% methanol in dichloromethane Rf: 0.2 LCMS (ESI): m/z 608.7 [M+H]⁺

### N-((2S)-1-((4-cyano-1-(8-isobutyryl-2-oxo-1,8-diazaspiro[4.5]decan-3-yl)-3-oxo-4-(tetrahydro-1l4-thiophen-1-ylidene)butan-2-yl)amino)-3-cyclohexyl-1-oxopropan-2-yl)-1H-indole-2-carboxamide (6)

To a stirred solution of 2-((S)-3-cyclohexyl-2-(1H-indole-2-carboxamido)propanamido)-3-(8-isobutyryl-2-oxo-1,8-diazaspiro[4.5]decan-3-yl)propanoic acid (4) (150 mg, 0.26 mmol) THF (3 mL) added HATU (140 mg, 0.37 mmol), DIPEA (0.13 mL, 0.74 mmol) and 1-(cyanomethyl)tetra hydro-1H-thiophen-1-iumbromide (5) (101 mg, 0.49 mmol) at 0° C. simultaneously and stirred at room temperature for 2 h. Reaction mixture was diluted with ice water (15 mL), extracted with dichloromethane (2×20 mL), dried over sodium sulfate and evaporated under reduced pressure. The crude residue was purified by combi-flash NP, compound eluted at 5% methanol in dichloromethane to afford N-((2S)-1-((4-cyano-1-(8-isobutyryl-2-oxo-1,8-diazaspiro[4.5]decan-3-yl)-3-oxo-4-(tetrahydro-1l4-thiophen-1-ylidene)butan-2-yl)amino)-3-cyclohexyl-1-oxopropan-2-yl)-1H-indole-2-carboxamide (6) (150 mg, 0.21 mmol, 88% yield) as yellow color solid. TLC system: 10% Methanol in dichloromethane Rf: 0.5 LCMS (ESI): m/z 717.8 [M+H]⁺

### N-((2S)-1-((4-amino-1-(8-isobutyryl-2-oxo-1,8-diazaspiro[4.5]decan-3-yl)-3,4-dioxobutan-2-yl)amino)-3-cyclohexyl-1-oxopropan-2-yl)-1H-indole-2-carboxamide (Compound C138)

To a stirred solution of N-((2S)-1-((4-cyano-1-(8-isobutyryl-2-oxo-1,8-diazaspiro[4.5]decan-3-yl)-3-oxo-4-(tetrahydro-1l4-thiophen-1-ylidene)butan-2-yl)amino)-3-cyclohexyl-1-oxopropan-2-yl)-1H-indole-2-carboxamide (6) (150 mg, 0.32 mmol) in methanol (2 mL) was added m-CPBA (90 mg, 0.52 mmol) at 0° C. and the reaction mixture stirred for 2 h at 0° C. and added aq ammonia (1.1 mL) and stirred at RT for 2 h. The progress of the reaction was monitored by TLC and LCMS. Reaction mixture was quenched with sat. NaHCO₃ solution (15 mL) and extracted with DCM (2×15 mL). Organic layer was washed with brine solution (15 mL), dried over Na₂SO₄ and concentrated to get crude compound. The crude compound was purified by prep HPLC to afford N-((2S)-1-((4-amino-1-(8-isobutyryl-2-oxo-1,8-diazaspiro[4.5]decan-3-yl)-3,4-dioxobutan-2-yl)amino)-3-cyclohexyl-1-oxopropan-2-yl)-1H-indole-2-carboxamide (Compound C138) (7 mg, 0019 mmol, 5% yield) as an off white solid. TLC system: 10% Methanol in dichloromethane Rf: 0.3 LCMS (ESI): m/z 635.2 [M+H]⁺

### Example 108: Synthesis of Compound C139

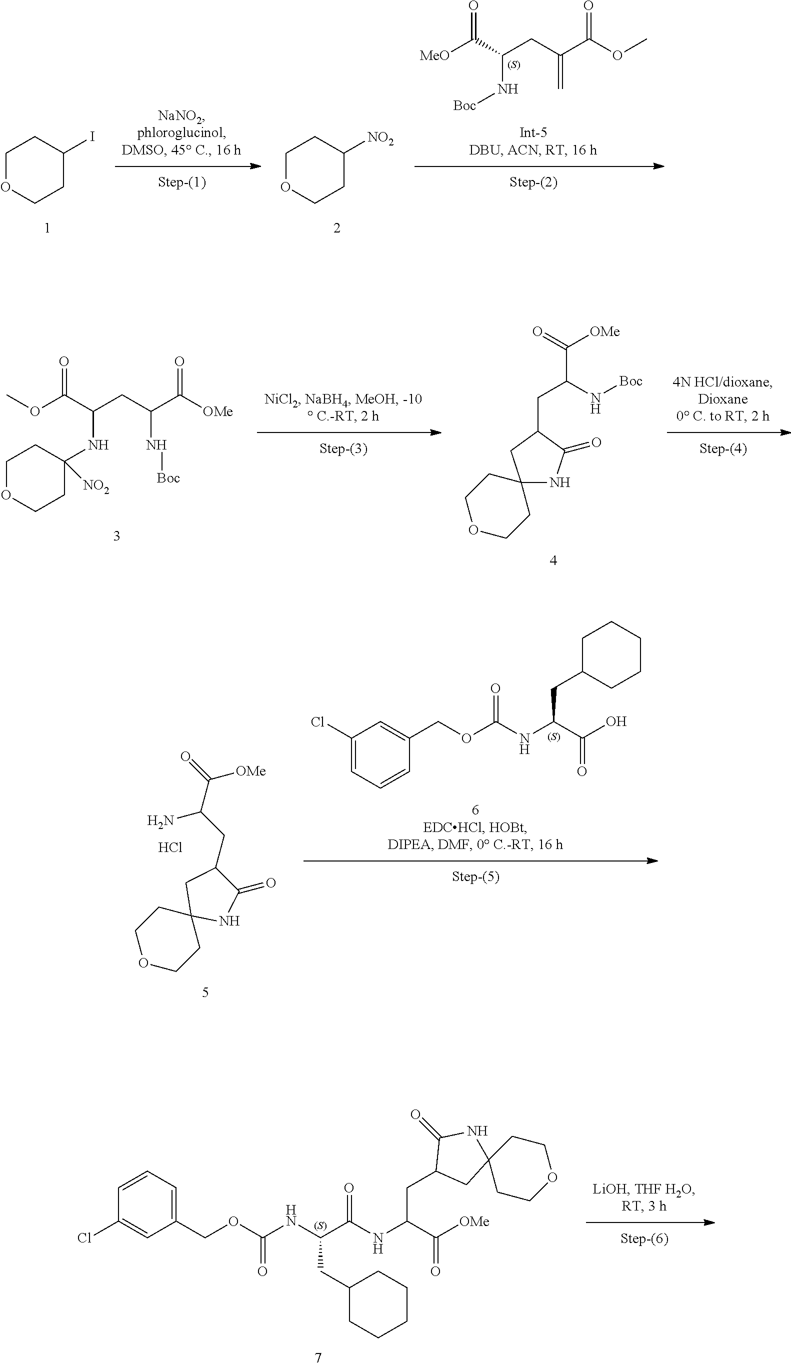

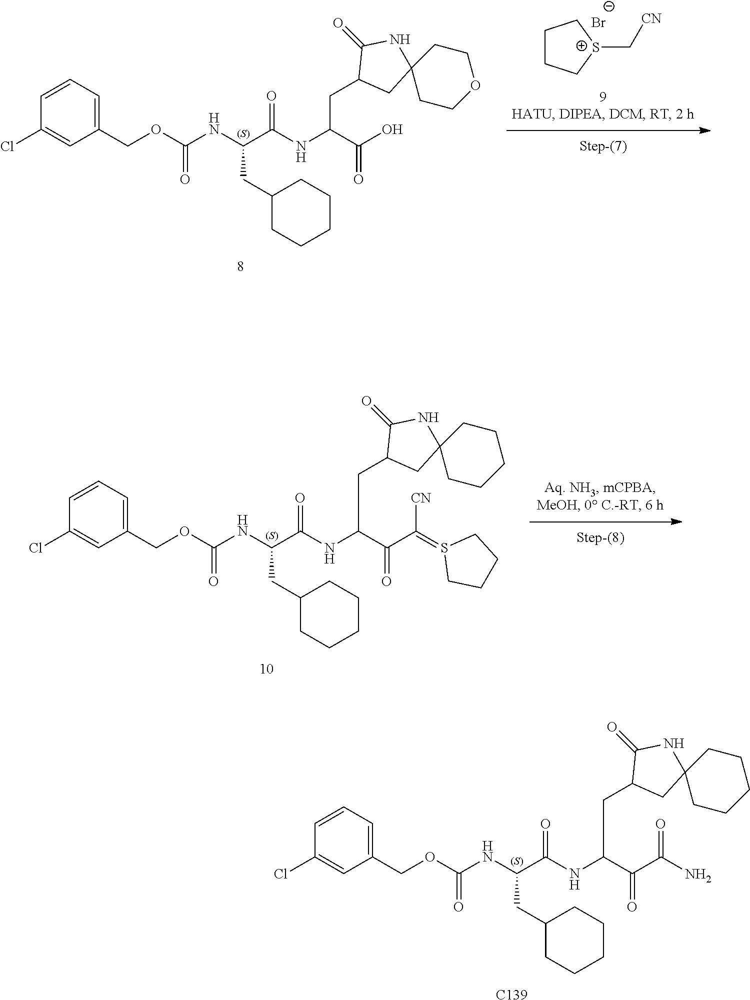

### 4-Nitrotetrahydro-2H-pyran (2)

To a stirred solution of 4-iodotetrahydro-2H-pyran (1) (10.0 g, 47.1 mmol) DMF (250 mL) added NaNO₂ (6.4 g, 94.3 mmol), phloroglucinol (9.5 g, 75.4 mmol), DMSO (100 mL) was added and heated at 45° C. for 16 h. Reaction mixture was diluted with ice water (80 mL), extracted with diethyl ether (2×60 mL), dried over sodium sulfate and evaporated under reduced pressure. The crude residue was purified by combi-flash NP by eluting at 20% in ethyl acetate and hexane afforded 4-nitrotetrahydro-2H-pyran (2). TLC system: 20% Ethyl acetate in hexane Rf: 0.4 LCMS (ESI): m/z 131.92 [M+H]⁺

### Dimethyl 2-((tert-butoxycarbonyl)amino)-4-((4-nitrotetrahydro-2H-pyran-4-yl)methyl)pentanedioate (3)

To a stirred solution of 4-nitrotetrahydro-2H-pyran (2) (2.1 g, 16.0 mmol) in ACN (20 mL), water (20 mL), dimethyl (S)-2-((tert-butoxycarbonyl)amino)-4-methylenepentanedioate (5.06 g, 17.6 mmol) and DBU (7.3 mL, 48.0 mmol) was added and stirred at room temperature for 16 h. The progress of the reaction was monitored by TLC and LCMS. Reaction mixture completely distilled under reduced pressure, crude compound extracted with ethyl acetate (2×50 mL), dried over sodium sulfate and concentrated under reduced pressure. The crude residue was purified by combi-flash NP by eluting at 30% ethyl acetate and hexane afforded dimethyl 2-((tert-butoxycarbonyl)amino)-4-((4-nitrotetrahydro-2H-pyran-4-yl)methyl)pentanedioate (3). TLC system: 30% Ethyl acetate in hexane Rf: 0.2 LCMS (ESI): m/z 441.25 [M+Na]⁺

### Methyl 2-((tert-butoxycarbonyl)amino)-3-(2-oxo-8-oxa-1-azaspiro[4.5]decan-3-yl)propanoate (4)

To a stirred solution of 2-((tert-butoxycarbonyl)amino)-4-((4-nitrotetrahydro-2H-pyran-4-yl)methyl)pentanedioate (3) (5.5 g, 13.15 mmol) in MeOH (250 mL) was added NiCl₂ (1.69 g, 13.15 mmol) and stirred at −10° C. for 10 min. To this was added NaBH₄ (2.5 g, 65.7 mmol) and stirred for 2 h. After completion of reaction by TLC, concentrated the reaction mixture, quenched with sat. NH₄Cl, filtered through calcite pad and the filtrate was extracted with 10% methanol in DCM (2×60 mL), dried over sodium sulfate and evaporated under reduced to afford methyl 2-((tert-butoxycarbonyl)amino)-3-(2-oxo-8-oxa-1-azaspiro[4.5]decan-3-yl)propanoate (4). TLC system: 100% Ethyl acetate Rf: 0.4 LCMS (ESI): m/z 379.1[M+Na]⁺

### Methyl 2-amino-3-(2-oxo-8-oxa-1-azaspiro[4.5]decan-3-yl)propanoate hydrochloride (5)

To a stirred solution of methyl 2-((tert-butoxycarbonyl)amino)-3-(2-oxo-8-oxa-1-azaspiro[4.5]decan-3-yl)propanoate (4) (3.8 g, 10.67 mmol) in 1,4 dioxane (30 mL), was added 4N HCl/dioxane (30 mL) at 0° C. and stirred at room temperature for 3 h. The progress of the reaction was monitored by TLC. Reaction mixture completely distilled under reduced pressure, crude compound was washed with diethyl ether to afford methyl 2-amino-3-(2-oxo-8-oxa-1-azaspiro[4.5]decan-3-yl)propanoate hydrochloride (5). TLC system: 100% Ethyl acetate Rf: 0.2 LCMS (ESI): m/z 257.24 [M+H]⁺

### Methyl 2-((S)-2-((((3-chlorobenzyl)oxy)carbonyl)amino)-3-cyclohexylpropanamido)-3-(2-oxo-8-oxa-1-azaspiro[4.5]decan-3-yl)propanoate (7)

To a stirred solution of (S)-2-((((3-chlorobenzyl)oxy)carbonyl)amino)-3-cyclohexylpropanoic acid (6) (1.5 g, 4.7 mmol) in DMF (15 mL), was added EDC.HCl (1.35 g, 7.1 mmol), HOBt (0.95 g, 7.1 mmol), DIPEA (2.6 mL, 14.1 mmol) and methyl 2-amino-3-(2-oxo-8-oxa-1-azaspiro[4.5]decan-3-yl)propanoate hydrochloride (5) (1.44 g, 4.7 mmol) at 0° C. subsequently and stirred at room temperature for 16 h. Reaction mixture was diluted with ice water (20 mL), extracted with ethyl acetate (2×60 mL), dried over sodium sulfate and evaporated under reduced pressure. The crude residue was purified by combi-flash NP by eluting at 5% methanol in dichloromethane to methyl 2-((S)-2-((((3-chlorobenzyl)oxy)carbonyl)amino)-3-cyclohexylpropanamido)-3-(2-oxo-8-oxa-1-azaspiro[4.5]decan-3-yl)propanoate (7). TLC system: 5% Methanol in DCM Rf: 0.5 LCMS (ESI): m/z 578.59 [M+H]⁺

### 2-((S)-2-((((3-Chlorobenzyl)oxy)carbonyl)amino)-3-cyclohexylpropanamido)-3-(2-oxo-8-oxa-1-azaspiro[4.5]decan-3-yl)propanoic acid (8)

To a stirred solution of methyl 2-((S)-2-((((3-chlorobenzyl)oxy)carbonyl)amino)-3-cyclohexylpropanamido)-3-(2-oxo-8-oxa-1-azaspiro[4.5]decan-3-yl)propanoate (7) (0.700 g, 1.2 mmol) in THF (10 mL), water (5 mL) was added lithium hydroxide (0.152 g, 3.6 mmol) at RT and continued stirring for 3 h. The progress of the reaction was monitored by TLC and LCMS. Reaction mixture was completely distilled under reduced pressure, crude compound acidified with aq. 1N HCl solution up to pH˜3 and extracted with ethyl acetate (2×10 mL), dried over sodium sulfate, concentrated under reduced pressure to afford 2-((S)-2-((((3-chlorobenzyl)oxy)carbonyl)amino)-3-cyclohexylpropanamido)-3-(2-oxo-8-oxa-1-azaspiro[4.5]decan-3-yl)propanoic acid (8). TLC system: 5% Methanol in DCM Rf: 0.2 LCMS (ESI): m/z 564.6[M+H]⁺

### 3-Chlorobenzyl ((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (10)

To a stirred solution of 2-((S)-2-((((3-chlorobenzyl)oxy)carbonyl)amino)-3-cyclohexylpropanamido)-3-(2-oxo-8-oxa-1-azaspiro[4.5]decan-3-yl)propanoic acid (8) (0.550 g, 0.97 mmol) in DCM (20 mL) was added HATU (0.555 g, 1.4 mmol), DIPEA (0.5 mL, 2.92 mmol) and 1-(cyanomethyl)tetrahydro-1H-thiophen-1-iumbromide (9) (0.315 g, 1.46 mmol) at 0° C. subsequently and continued stirring at RT for 2 h. Reaction mixture was diluted with ice water (30 mL), extracted with dichloromethane (2×10 mL), dried over sodium sulfate and evaporated under reduced pressure. The crude residue was purified by combi-flash NP, compound eluted at 5% methanol in dichloromethane to afford 3-chlorobenzyl ((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (10). TLC system: 10% Methanol in dichloromethane Rf: 0.6 LCMS (ESI): m/z 673.4 [M+H]⁺

### 3-Chlorobenzyl ((2S)-1-((4-amino-3,4-dioxo-1-(2-oxo-1-azaspiro[4.5]decan-3-yl)butan-2-yl)amino)-3-cyclohexyl-1-oxopropan-2-yl)carbamate (Compound C139)

To a stirred solution of 3-chlorobenzyl ((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (10) (0.270 g, 0.40 mmol) in methanol (10 mL) was added mCPBA (206.93 mg, 1.2 mmol) at 0° C. and the reaction mixture was stirred for 2 h at 0° C. and added aq ammonia (2.0 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep HPLC to afford 3-chlorobenzyl ((2S)-1-((4-amino-3,4-dioxo-1-(2-oxo-1-azaspiro[4.5]decan-3-yl)butan-2-yl)amino)-3-cyclohexyl-1-oxopropan-2-yl)carbamate (Compound C139). TLC system: 10% Methanol in dichloromethane Rf: 0.2 LCMS (ESI): m/z 591.1 [M+H]⁺

### Example 109: Synthesis of Compound C140

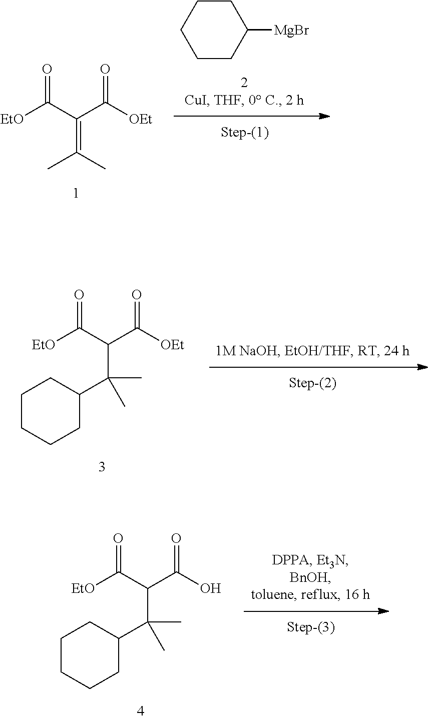

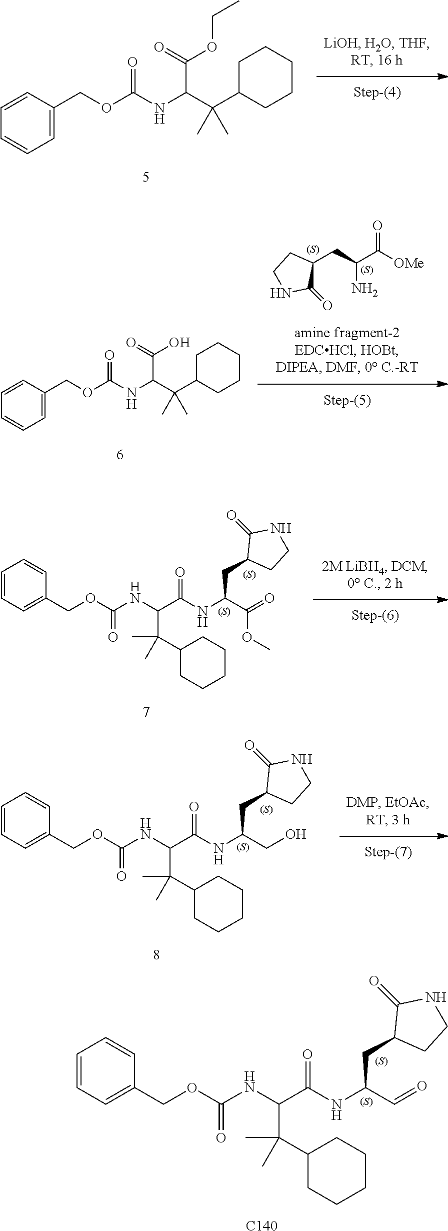

### Diethyl 2-(2-cyclohexylpropan-2-yl)malonate (3)

A solution of diethyl 2-(propan-2-ylidene)malonate (1) (5 g, 25.00 mmol), in THF (100 mL) was cooled to 0° C., followed by copper (I) iodide (7.1 g, 37.00 mmol). The mixture was stirred at 0° C. for 0.5 h. Then cyclohexyl magnesium bromide 1.0 M in THF (75 mL, 75.00 mmol) was added drop-wise into the above mixture at 0° C. The mixture was stirred at 0° C. for 2 h. Progress of the reaction was monitored by TLC and LCMS. The mixture was quenched with 1N HCl and extracted with ethyl acetate (2×100 mL) and washed with water (2×100 mL), dried over sodium sulfate, concentrated under reduced pressure to afford diethyl 2-(2-cyclohexylpropan-2-yl)malonate (3). TLC system: 5% Ethyl acetate in Pet ether R_{f}: 0.6 LCMS (ESI): m/z 471.27 [M+Na]⁺

### 3-Cyclohexyl-2-(ethoxycarbonyl)-3-methylbutanoic acid (4)

A stirred solution of diethyl 2-(2-cyclohexylpropan-2-yl)malonate (3) (4.5 g, 15.84 mmol) in mixture of ethanol (100 mL) and THF (50 mL), was treated with 1N solution of NaOH (25 mL, 15.84 mmol) and the reaction mixture was stirred at room temperature for 24 h. Progress of the reaction was monitored by TLC and LCMS. After 24 h, the mixture was evaporated to syrup and dissolved in water (200 mL) and extracted with ethyl ether (2×100 mL). The aqueous phase was acidified with 1N HCl to pH 2.0 and extracted with EtOAc (2×100 mL). The combined organic layers were washed with brine solution (200 mL), dried over Na₂SO₄ and concentrated to afford 3-cyclohexyl-2-(ethoxycarbonyl)-3-methylbutanoic acid (4). TLC system: 50% EtOAc in Pet ether Rf: 0.1 LCMS (ESI): m/z 279.29 [M+Na]⁺

### Ethyl 2-(((benzyloxy)carbonyl)amino)-3-cyclohexyl-3-methylbutanoate (5)

To a stirred solution of 3-cyclohexyl-2-(ethoxycarbonyl)-3-methylbutanoic acid (4) (3 g, 11.718 mmol) in dry benzene (30 mL), triethylamine (3.31 mL, 23.436 mmol) and diphenylphosphoryl azide (3.77 mL, 17.578 mmol) were added. The reaction mixture was heated at reflux for 2 h. Progress of the reaction was monitored by TLC and LCMS. After 2 h, the reaction mixture was cooling to room temperature, benzyl alcohol (1.8 mL, 17.57 mmol) was added and the reaction was heated again at reflux for 16 h. After evaporation of the solvent, the crude material was quenched with 5% citric acid solution and extracted with ethyl acetate (2×50 mL). The combined organic layers were washed with brine solution (50 mL), dried over Na₂SO₄ and concentrated and the crude was purified by flash chromatography to afford ethyl 2-(((benzyloxy)carbonyl)amino)-3-cyclohexyl-3-methylbutanoate (5). TLC system: 5% EtOAc in Pet ether Rf: 0.5 LCMS (ESI): m/z 362.36 [M+H]⁺

### 2-(((Benzyloxy)carbonyl)amino)-3-cyclohexyl-3-methylbutanoic acid (6)

To a stirred solution of ethyl 2-(((benzyloxy)carbonyl)amino)-3-cyclohexyl-3-methylbutanoate (5) (3.2 g, 8.31 mmol) in MeOH/THF (15 mL), water (5 mL) was added lithium hydroxide (1.1 g, 26.59 mmol) at RT and stirred at room temperature for 16 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), The combined organic layers were washed with brine solution (20 mL), dried over Na₂SO₄ and concentrated to afford 2-(((benzyloxy)carbonyl)amino)-3-cyclohexyl-3-methylbutanoic acid (6). TLC system: 10% MeOH in DCM Rf: 0.1 LCMS (ESI): m/z 334.37 [M+H]⁺

### Methyl (2S)-2-(2-(((benzyloxy)carbonyl)amino)-3-cyclohexyl-3-methylbutanamido)-3-((S)-2-oxopyrrolidin-3-yl)propanoate (7)

To a stirred solution of 2-(((benzyloxy)carbonyl)amino)-3-cyclohexyl-3-methylbutanoic acid (6) (2.4 g, 7.207 mmol) in DMF (25 mL) was added EDC.HCl (2.06 g, 10.81 mmol), HOBt (1.46 g, 10.81 mmol), and DIPEA (3.87 mL, 21.6216 mmol) at 0° C., then methyl 2-amino-2-methyl-3-(2-oxopyrrolidin-3-yl)propanoate hydrochloride (amine fragment-2A) (1.9 g, 8.64 mmol) at 0° C. and stirred at room temperature for 16 h. Progress of the reaction was monitored by TLC and LCMS. Reaction mixture was diluted with ice water (50 mL), extracted with ethyl acetate (2×50 mL), dried over sodium sulfate and evaporated under reduced pressure. The crude was purified by flash chromatography to afford methyl (2S)-2-(2-(((benzyloxy)carbonyl)amino)-3-cyclohexyl-3-methylbutanamido)-3-((S)-2-oxopyrrolidin-3-yl)propanoate (7). TLC system: 5% MeOH in DCM Rf: 0.3 LCMS (ESI): m/z 502.60 [M+H]⁺

### Benzyl (3-cyclohexyl-1-(((S)-1-hydroxy-3-((S)-2-oxopyrrolidin-3-yl)propan-2-yl)amino)-3-methyl-1-oxobutan-2-yl)carbamate (8)

To a stirred solution of methyl (2S)-2-(2-(((benzyloxy)carbonyl)amino)-3-cyclohexyl-3-methylbutanamido)-3-((S)-2-oxopyrrolidin-3-yl)propanoate (7) (500 mg, 0.998 mmol) in DCM (5 mL) was added 2M LiBH₄ in THF (0.99 mL, 1.996 mmol) at 0° C. and the reaction mixture stirred for 2 h at 0° C. The progress of the reaction was monitored by TLC and LCMS. Reaction mixture was quenched with sat. ammonium chloride solution (30 mL) and extracted with DCM (2×30 mL). Organic layer was washed with brine solution (20 mL), dried over Na₂SO₄ and concentrated to afford benzyl (3-cyclohexyl-1-(((S)-1-hydroxy-3-((S)-2-oxopyrrolidin-3-yl)propan-2-yl)amino)-3-methyl-1-oxobutan-2-yl)carbamate (8). TLC system: 10% MeOH in DCM Rf: 0.4 LCMS (ESI): m/z 474.46 [M+H]⁺

### Benzyl (3-cyclohexyl-3-methyl-1-oxo-1-(((S)-1-oxo-3-((S)-2-oxopyrrolidin-3-yl)propan-2-yl)amino)butan-2-yl)carbamate (Compound 0140)

To a stirred solution of benzyl (3-cyclohexyl-1-(((S)-1-hydroxy-3-((S)-2-oxopyrrolidin-3-yl)propan-2-yl)amino)-3-methyl-1-oxobutan-2-yl)carbamate (8) (200 mg, 0.422 mmol) in ethyl acetate (5 mL) was added Dess-Martin periodinane (268 mg, 0.634 mmol) at 0° C. and stirred at RT for 16 h. The progress of the reaction was monitored by TLC and LCMS. After complete consumption of the starting material by TLC and LCMS, the reaction mass was filtered through celite pad and the celite pad thoroughly was with ethyl acetate (30 mL). Then the organic layer was washed with 10% sodium thiosulfate solution (2×50 mL) followed by saturated sodium bicarbonate solution (2×50 mL), water (1×50 mL), brine (1×50 mL). Then the organic layer was dried over sodium sulfate and evaporated under vacuum. Then the crude compound was purified by Prep-HPLC purification to get benzyl (3-cyclohexyl-3-methyl-1-oxo-1-(((S)-1-oxo-3-((S)-2-oxopyrrolidin-3-yl)propan-2-yl)amino)butan-2-yl)carbamate (Compound C140). TLC system: 10% MeOH in DCM Rf: 0.45 LCMS (ESI): m/z 472.2 (M+H)⁺

### Example 110: Synthesis of Compound C143

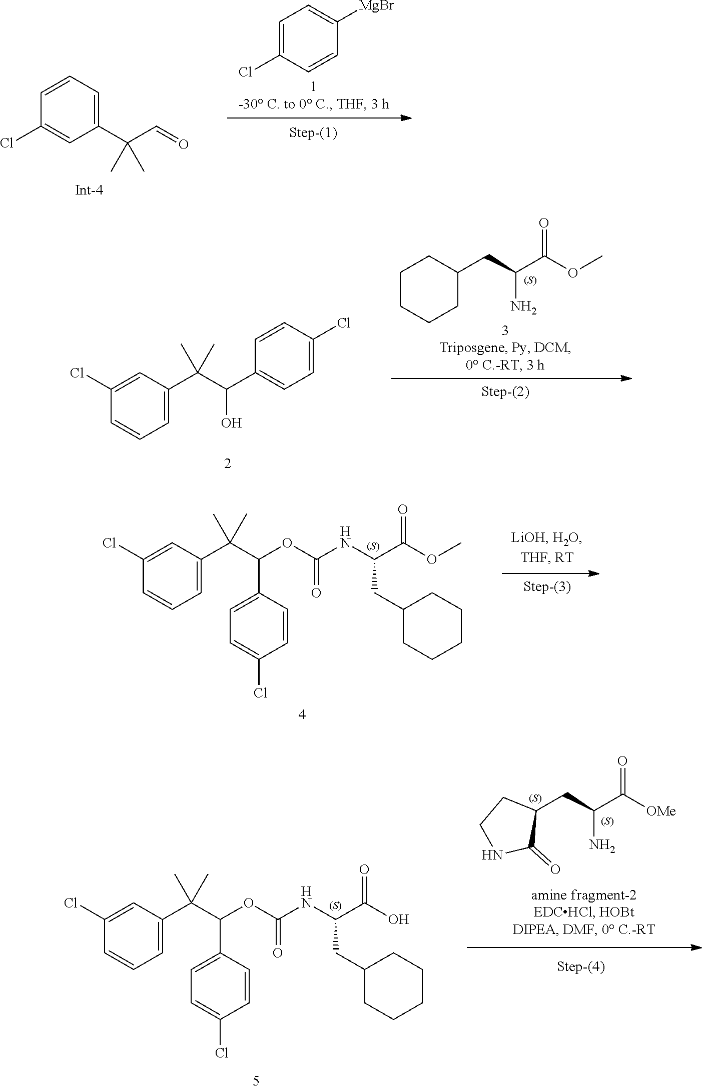

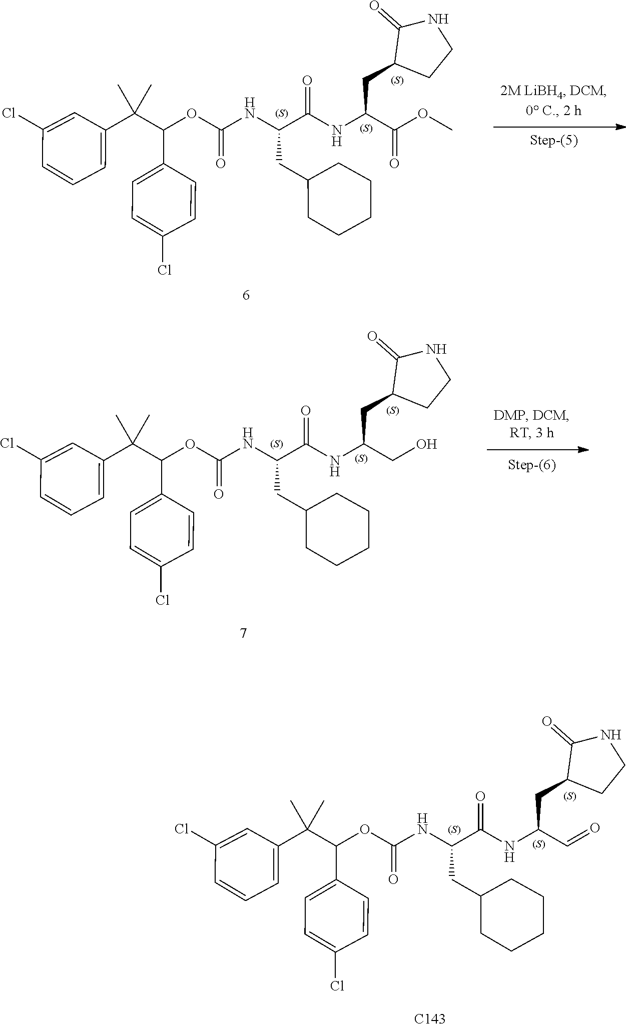

### 2-(3-Chlorophenyl)-1-(4-chlorophenyl)-2-methylpropan-1-ol (2)

To a stirred solution of 2-(3-chlorophenyl)-2-methylpropanal (Int-4) (5 g, 27.472 mmol) in THF (50 mL) was added (4-chlorophenyl)magnesium bromide (54 mL, 54.945 mmol) at −30° C. and the reaction mixture was stirred at 0° C. for 3 h. The progress of the reaction was monitored by TLC. The reaction mixture was quenched with saturated NH₄Cl (50 mL) and extracted with ethyl acetate (2×50 mL), dried over sodium sulfate and evaporated under reduced pressure. The crude residue was purified by silica gel column by eluting with 10% ethyl acetate in hexane to afford 2-(3-chlorophenyl)-1-(4-chlorophenyl)-2-methylpropan-1-ol (2). TLC system: 10% Ethyl acetate in hexane Rf: 0.3 LCMS (ESI): m/z=277.19 [M−OH]

### Methyl (2S)-2-(((2-(3-chlorophenyl)-1-(4-chlorophenyl)-2-methylpropoxy) carbonyl) amino)-3-cyclohexylpropanoate (4)

To a stirred solution of 2-(3-chlorophenyl)-1-(4-chlorophenyl)-2-methylpropan-1-ol (2) (3 g, 10.20 mmol) in DCM (30 mL) was added pyridine (3 mL) methyl (S)-2-amino-3-cyclohexylpropanoate hydrochloride (3) (2.4 g, 13.265 mmol) followed by triphosgene (1.5 g, 5.102 mmol) at 0° C. with portion wise and stirred at RT for 16 h. The progress of the reaction was monitored by TLC. Reaction mixture was quenched with 1N aq. HCl (50 mL) then extracted with DCM (2×50 mL), washed with sat. NaHCO₃ solution (2×50 mL), dried over sodium sulfate and evaporated under reduced pressure. The crude residue was purified by silica gel column by eluting with 15% ethyl acetate in hexane to afford methyl (2S)-2-(((2-(3-chlorophenyl)-1-(4-chlorophenyl)-2-methylpropoxy) carbonyl) amino)-3-cyclohexylpropanoate (4). TLC system: 10% Ethyl acetate in hexane Rf: 0.3 LCMS (ESI): m/z=528.52 [M+Na]⁺

### (2S)-2-(((2-(3-Chlorophenyl)-1-(4-chlorophenyl)-2-methylpropoxy) carbonyl) amino)-3-cyclohexylpropanoic acid (5)

To a stirred solution of methyl (2S)-2-(((2-(3-chlorophenyl)-1-(4-chlorophenyl)-2-methylpropoxy)carbonyl) amino)-3-cyclohexylpropanoate (4) (4.2 g, 8.313 mmol) in THF (15 mL), water (10 mL) was added LiOH.H₂O (1 g, 24.940 mmol) at 0° C. Reaction mixture was stirred at RT for 3 h. The progress of the reaction was monitored by TLC. After consumption of starting material, the reaction mixture was concentrated and acidified with 1N HCl, extracted with ethyl acetate (2×50 mL), dried over sodium sulfate and evaporated under reduced pressure to afford (2S)-2-(((2-(3-chlorophenyl)-1-(4-chlorophenyl)-2-methylpropoxy) carbonyl)amino)-3-cyclohexylpropanoic acid (5) which was used directly in the next step. TLC system: 10% Methanol in dichloromethane Rf: 0.2 LCMS (ESI): m/z=514.45 [M+Na]⁺

### Methyl (2S)-2-((2S)-2-(((2-(3-chlorophenyl)-1-(4-chlorophenyl)-2-methylpropoxy) carbonyl) amino)-3-cyclohexylpropanamido)-3-((S)-2-oxopyrrolidin-3-yl) propanoate (6)

To a stirred solution of (2S)-2-(((2-(3-chlorophenyl)-1-(4-chlorophenyl)-2-methylpropoxy)carbonyl)amino)-3-cyclohexylpropanoic acid (5) (4 g, 8.143 mmol) in DMF (35 mL) was added EDC.HCl (2.3 g, 12.219 mmol), HOBt (1.6 g, 12.219 mmol), DIPEA (3.5 mL, 24.429 mmol) and methyl (S)-2-amino-3-((S)-2-oxopyrrolidin-3-yl)propanoate hydrochloride (amine-fragment-2) (1.8 g, 9.775 mmol) at 0° C. simultaneously and stirred at room temperature for 16 h. Reaction mixture was diluted with ice water (50 mL), extracted with ethyl acetate (2×50 mL), the organic layer was dried over sodium sulfate and evaporated under reduced pressure to afford crude compound. The crude residue was purified by silica gel column by eluting with 80% ethyl acetate and pet-ether to afford methyl (2S)-2-((2S)-2-(((2-(3-chlorophenyl)-1-(4-chlorophenyl)-2-methylpropoxy)carbonyl)amino)-3-cyclohexylpropanamido)-3-((S)-2-oxo pyrrolidin-3-yl) propanoate (6). TLC system: 10% Methanol in dichloromethane Rf: 0.3 LCMS (ESI): m/z=660.47 [M+H]⁺

### Methyl (2S)-2-((2S)-2-(((2-(3-chlorophenyl)-1-(4-chlorophenyl)-2-methylpropoxy) carbonyl) amino)-3-cyclohexylpropanamido)-3-((S)-2-oxopyrrolidin-3-yl) propanoate (7)

To a stirred solution of methyl (2S)-2-((2S)-2-(((2-(3-chlorophenyl)-1-(4-chlorophenyl)-2-methylpropoxy)carbonyl)amino)-3-cyclohexylpropanamido)-3-((S)-2-oxopyrrolidin-3-yl) propanoate (6) (700 mg, 1.062 mmol) in DCM (7 mL) was added 2M LiBH₄ in THF (1 mL, 4.878 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to get compound to afford 2-(3-chlorophenyl)-1-(4-chlorophenyl)-2-methylpropyl((S)-3-cyclohexyl-1-(((S)-1-hydroxy-3-((S)-2-oxopyrrolidin-3-yl) propan-2-yl)amino)-1-oxopropan-2-yl) carbamate (7). TLC system: 10% Methanol in DCM Rf: 0.4 LCMS (ESI): m/z 632.65 (M+H)⁺

### 2-(3-Chlorophenyl)-1-(4-chlorophenyl)-2-methylpropyl ((S)-3-cyclohexyl-1-oxo-1-(((S)-1-oxo-3-((S)-2-oxopyrrolidin-3-yl) propan-2-yl) amino) propan-2-yl) carbamate (Compound C143)

To a stirred solution of 2-(3-chlorophenyl)-1-(4-chlorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl) carbamate (7) (200 mg, 0.316 mmol) was dissolved in DCM (6 mL) was added Dess-Martin periodinane (403 mg, 0.95 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by prep HPLC to afforded 2-(3-chlorophenyl)-1-(4-chlorophenyl)-2-methylpropyl((S)-3-cyclohexyl-1-oxo-1-(((S)-1-oxo-3-((S)-2-oxopyrrolidin-3-yl) propan-2-yl)amino)propan-2-yl)carbamate (Compound C143). TLC system: 10% Methanol in DCM Rf: 0.5 LCMS (ESI): m/z 630.1 (M+H)⁺

### Example 111: Synthesis of Compound C144

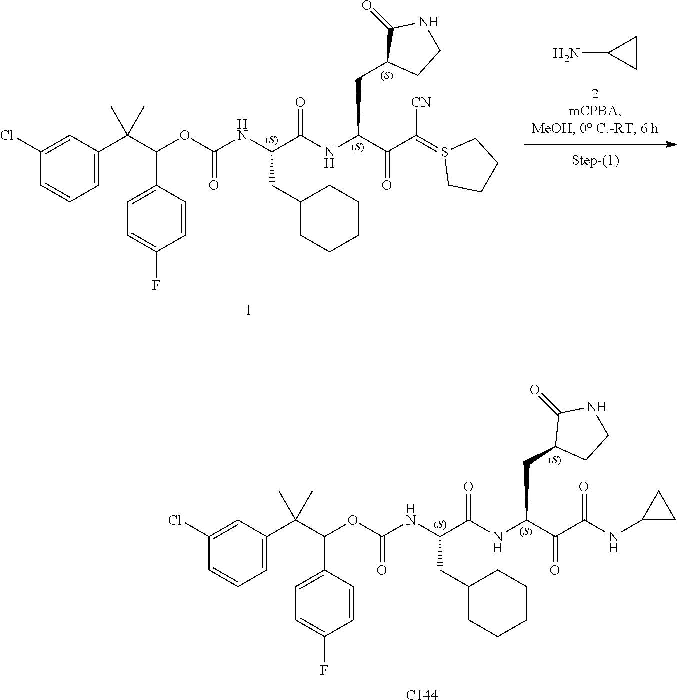

### 2-(3-Chlorophenyl)-1-(4-fluorophenyl)-2-methylpropyl ((S)-3-cyclohexyl-1-(((S)-4-(cyclopropylamino)-3,4-dioxo-1-((S)-2-oxopyrrolidin-3-yl) butan-2-yl) amino)-1-oxopropan-2-yl) carbamate (Compound C144)

To a stirred solution of 2-(3-chlorophenyl)-1-(4-fluorophenyl)-2-methylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (1) (150 mg, 0.203 mmol) in methanol (2 mL) was added m-CPBA (69 mg, 0.406 mmol) at 0° C. and the reaction mixture stirred for 2 h at 0° C. and added cyclopropanamine (1 mL) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over sodium sulfate and concentrated to get crude compound. The crude compound was purified by prep-HPLC to afford 2-(3-chlorophenyl)-1-(4-fluorophenyl)-2-methylpropyl ((S)-3-cyclohexyl-1-(((S)-4-(cyclopropyl amino)-3,4-dioxo-1-((S)-2-oxopyrrolidin-3-yl) butan-2-yl)amino)-1-oxopropan-2-yl) carbamate (Compound C144). TLC system: 10% Methanol in dichloromethane Rf: 0.3 LCMS (ESI): m/z 697.2 [M+H]+

### Example 112: Synthesis of Compound C146

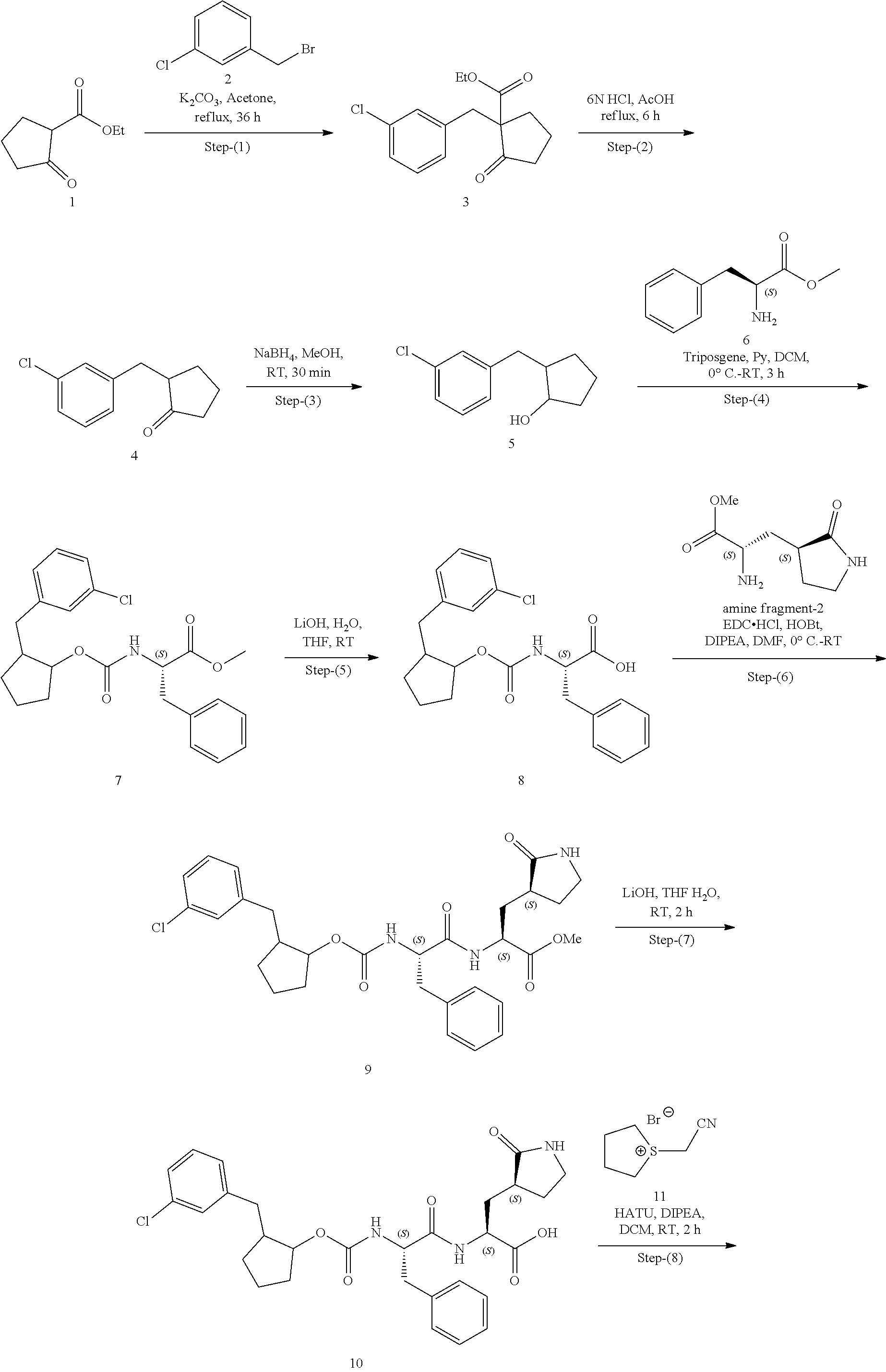

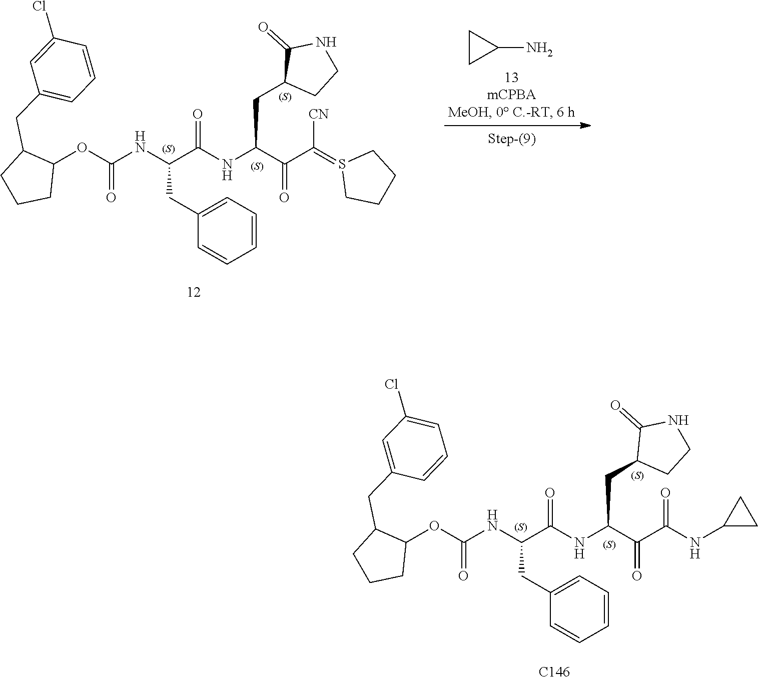

### Ethyl 1-(3-chlorobenzyl)-2-oxocyclopentane-1-carboxylate (3)

To a stirred solution of K₂CO₃ (17.6 g, 128.205 mmol) in acetone (100 mL) was added ethyl 2-oxocyclopentane-1-carboxylate (1) (10 g, 64.102 mmol) followed by 1-(bromo methyl)-3-chlorobenzene (9.6 mL, 76.923 mmol) at RT and the reaction mixture was refluxed at 70° C. for 36 h. The progress of the reaction was monitored by TLC. Reaction mixture was cooled to RT and quenched with saturated NaHCO₃ solution (150 mL) then extracted with ethyl acetate (2×100 mL). Organic layer was washed with saturated NaHCO₃ (3×100 mL), dried over sodium sulfate and evaporated under reduced pressure. The crude residue was purified by silica gel column by eluting with 3% ethyl acetate in hexane to afford ethyl 1-(3-chlorobenzyl)-2-oxocyclopentane-1-carboxylate (3) TLC system: 5% Ethyl acetate in hexane Rf: 0.5 LCMS (ESI): m/z=281.23 [M+H]⁺

### 2-(3-chlorobenzyl) cyclopentan-1-one (4)

To a stirred solution of ethyl 1-(3-chlorobenzyl)-2-oxocyclopentane-1-carboxylate (3) (15 g, 35.714 mmol) in glacial acetic acid (150 mL) was added 6N aq.HCl (75 mL) at RT and the reaction mixture was refluxed at 120° C. for 6 h. The progress of the reaction was monitored by TLC. Reaction mixture was cooled to RT and poured into ice-cold water (100 mL) and extracted with ethyl acetate (3×50 mL), dried over sodium sulfate and evaporated under reduced pressure. The crude residue was purified by silica gel column by eluting with 2% ethyl acetate in hexane to afford 2-(3-chlorobenzyl)cyclopentan-1-one (4). TLC system: 5% Ethyl acetate in hexane Rf: 0.5 LCMS (ESI): m/z=209.09 [M+H]⁺

### 2-(3-Chlorobenzyl) cyclopentan-1-ol (5)

To a stirred solution of 2-(3-chlorobenzyl) cyclopentan-1-one (4) (5 g, 24.038 mmol) in MeOH (50 mL) was added NaBH₄ (1.82 g, 48.076 mmol) at 0° C. Reaction mixture was allowed to RT and stirred for 30 min. The progress of the reaction was monitored by TLC. Reaction mixture was quenched with 1N HCl and evaporated to remove solvent then extracted with ethyl acetate (2×50 mL), dried over sodium sulfate, concentrated under reduced pressure to afford the crude compound 2-(3-chlorobenzyl)cyclopentan-1-ol (5). TLC system: 5% Ethyl acetate in hexane Rf: 0.2 LCMS (ESI): m/z=193.14 [M−OH]

### Methyl (((2-(3-chlorobenzyl)cyclopentyl)oxy)carbonyl)-L-phenylalaninate (7)

To a stirred solution of 2-(3-chlorobenzyl)cyclopentan-1-ol (5) (3 g, 14.28 mmol), methyl L-phenylalaninate HCl (6) (3 g, 16.75 mmol) in DCM (40 mL) was added pyridine (9 mL, 3 vol) followed by triphosgene (2.1 g, 7.09 mmol) at 0° C. and stirred at room temperature for 3 h. The progress of the reaction was monitored by TLC and LCMS. Reaction mixture was quenched with 2N HCl (30 mL), extracted with dichloromethane (2×50 mL). The combined organic layer was dried over sodium sulfate, filtered and evaporated under reduced pressure. The crude residue was purified by combi-flash, compound eluted at 10% ethyl acetate in pet ether to afford methyl (((2-(3-chlorobenzyl)cyclopentyl)oxy)carbonyl)-L-phenylalaninate (7). TLC system: 10% Ethyl acetate in Hexane Rf: 0.3 LCMS (ESI): m/z=416.4 [M+H]⁺

### (((2-(3-Chlorobenzyl)cyclopentyl)oxy)carbonyl)-L-phenylalanine (3)

To a stirred solution of methyl (((2-(3-chlorobenzyl)cyclopentyl)oxy)carbonyl)-L-phenylalaninate (7) (2.1 g, 5.06 mmol) in THF (20 mL), DM water (10 mL), LiOH.H₂O (622 mg, 15.12 mmol) was added. Reaction mixture was stirred at RT for 2 h. The progress of the reaction was monitored by TLC. After consumption of starting material, the reaction mixture was concentrated and acidified with 1N aq. HCl, extracted with ethyl acetate (2×50 mL), dried over sodium sulfate and evaporated under reduced pressure to afford (((2-(3-chlorobenzyl)cyclopentyl)oxy)carbonyl)-L-phenylalanine (8) which was used directly in the next step. TLC system: 5% Methanol in dichloromethane Rf: 0.2 LCMS (ESI): m/z=424.3 [M+Na]⁺

### Methyl (2S)-2-((2S)-2-((((2-(3-chlorobenzyl)cyclopentyl)oxy)carbonyl)amino)-4-methylpentanamido)-3-((S)-2-oxopyrrolidin-3-yl)propanoate (9)

To a stirred solution (((2-(3-chlorobenzyl)cyclopentyl)oxy)carbonyl)-L-phenylalanine (8) (1.8 g, 4.48 mmol), in DMF (20 mL) was added EDC.HCl (1.2 g, 6.73 mmol), HOBT (0.9 g, 6.73 mmol), DIPEA (2.4 mL, 13.46 mmol) and methyl (2S)-2-amino-3-(2-oxopyrrolidin-3-yl)propanoate (amine-fragment-2) (1.34 g, 5.422 mmol) at 0° C. simultaneously and stirred at room temperature for 16 h. Reaction mixture was diluted with ice water (50 mL), extracted with ethyl acetate (2×100 mL), dried over sodium sulfate and evaporated under reduced pressure. The crude residue was purified by silica gel column by eluting with 5% methanol in dichloromethane to afford methyl (2S)-2-((2S)-2-((((2-(3-chlorobenzyl)cyclopentyl)oxy)carbonyl)amino)-4-methylpentanamido)-3-((S)-2-oxopyrrolidin-3-yl)propanoate (9). TLC system: 10% Methanol in dichloromethane Rf: 0.4 LCMS (ESI): m/z=570.4 [M+H]⁺

### (2S)-2-((2S)-2-((((2-(3-Chlorobenzyl)cyclopentyl)oxy)carbonyl)amino)-3-phenylpropanamido)-3-((S)-2-oxopyrrolidin-3-yl)propanoic acid (10)

To a stirred solution of methyl methyl (2S)-2-((2S)-2-((((2-(3-chlorobenzyl)cyclopentyl)oxy)carbonyl)amino)-3-phenylpropanamido)-3-((S)-2-oxopyrrolidin-3-yl)propanoate (9) (1.1 g, 1.495 mmol) in THF (20 mL), DM water (10 mL), LiOH.H₂O (622 mg, 15.12 mmol) was added. Reaction mixture was stirred at RT for 2 h. The progress of the reaction was monitored by TLC. After consumption of starting material, the reaction mixture was concentrated and acidified with 1N aq. HCl, extracted with ethyl acetate (2×50 mL), dried over sodium sulfate and evaporated under reduced pressure to afford (2S)-2-((2S)-2-((((2-(3-chlorobenzyl)cyclopentyl)oxy)carbonyl)amino)-3-phenylpropanamido)-3-((S)-2-oxopyrrolidin-3-yl)propanoic acid (5). TLC system: 10% Methanol in dichloromethane Rf: 0.2 LCMS (ESI): m/z=556.4 [M+H]⁺

### 2-(3-Chlorobenzyl)cyclopentyl ((S)-1-(((S)-4-cyano-3-oxo-1-((S)-2-oxopyrrolidin-3-yl)-4-(tetrahydro-1l4-thiophen-1-ylidene)butan-2-yl)amino)-1-oxo-3-phenylpropan-2-yl)carbamate (12)

To a stirred solution of 2S)-2-((2S)-2-((((2-(3-chlorobenzyl)cyclopentyl)oxy)carbonyl)amino)-3-phenylpropanamido)-3-((S)-2-oxopyrrolidin-3-yl)propanoic acid (10) (500 mg, 8.99 mmol) DCM (10 mL) added HATU (680 mg, 1.33 mmol), DIPEA (0.4 mL, 4.41 mmol) and 1-(cyanomethyl) tetrahydro-1H-thiophen-1-iumbromide (11) (455 mg, 2.20 mmol) at 0° C. simultaneously and stirred at room temperature for 2 h. Reaction mixture was diluted with ice water (20 mL), extracted with dichloromethane (2×20 mL), dried over sodium sulfate and evaporated under reduced pressure. The crude residue was purified by combi-flash NP, compound eluted at 5% methanol in dichloromethane to afford ((S)-1-(((S)-4-cyano-3-oxo-1-((S)-2-oxopyrrolidin-3-yl)-4-(tetrahydro-1l4-thiophen-1-ylidene)butan-2-yl)amino)-1-oxo-3-phenylpropan-2-yl)carbamate (12). TLC system: 10% Methanol in dichloromethane Rf: 0.5 LCMS (ESI): m/z=665.4 [M+H]⁺

### 2-(3-Chlorobenzyl)cyclopentyl((S)-1-(((S)-4-(cyclopropylamino)-3,4-dioxo-1-((S)-2-oxopyrrolidin-3-yl)butan-2-yl)amino)-1-oxo-3-phenylpropan-2-yl)carbamate (Compound C146)

To a stirred solution of ((S)-1-(((S)-4-cyano-3-oxo-1-((S)-2-oxopyrrolidin-3-yl)-4-(tetrahydro-1l4-thiophen-1-ylidene)butan-2-yl)amino)-1-oxo-3-phenylpropan-2-yl)carbamate (12) (120 mg, 0.18 mmol) in methanol (3 mL) was added m-CPBA (62 mg, 0.36 mmol) at 0° C. and the reaction mixture stirred for 2 h at 0° C. and added cyclopropylamine (0.01 mL, 0.21 mmol) and stirred at RT for 4 h. The progress of the reaction was monitored by TLC and LCMS. Reaction mixture was quenched with sat. NaHCO₃ solution (20 mL) and extracted with DCM (2×20 mL). Organic layer was washed with brine solution (20 mL), dried over Na₂SO₄ and concentrated to get crude compound. The crude compound was purified by prep HPLC to afford 2-(3-chlorobenzyl)cyclopentyl((S)-1-(((S)-4-(cyclopropylamino)-3,4-dioxo-1-((S)-2-oxopyrrolidin-3-yl)butan-2-yl)amino)-1-oxo-3-phenylpropan-2-yl)carbamate (Compound C146). TLC system: 10% Methanol in dichloromethane Rf: 0.3 LCMS (ESI): m/z 623.1 (M+H)⁺

### Example 113: Synthesis of Compound C147

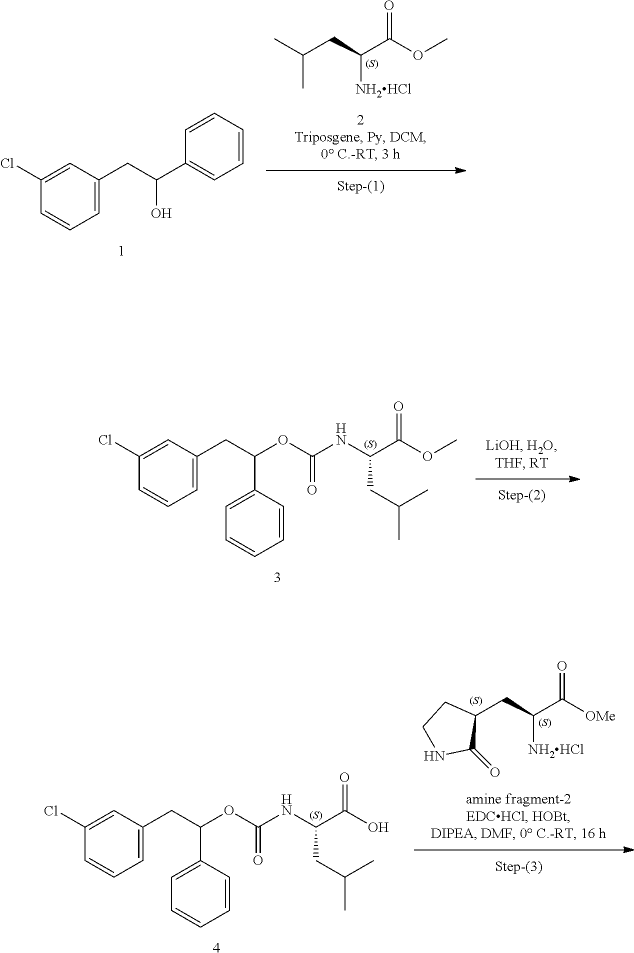

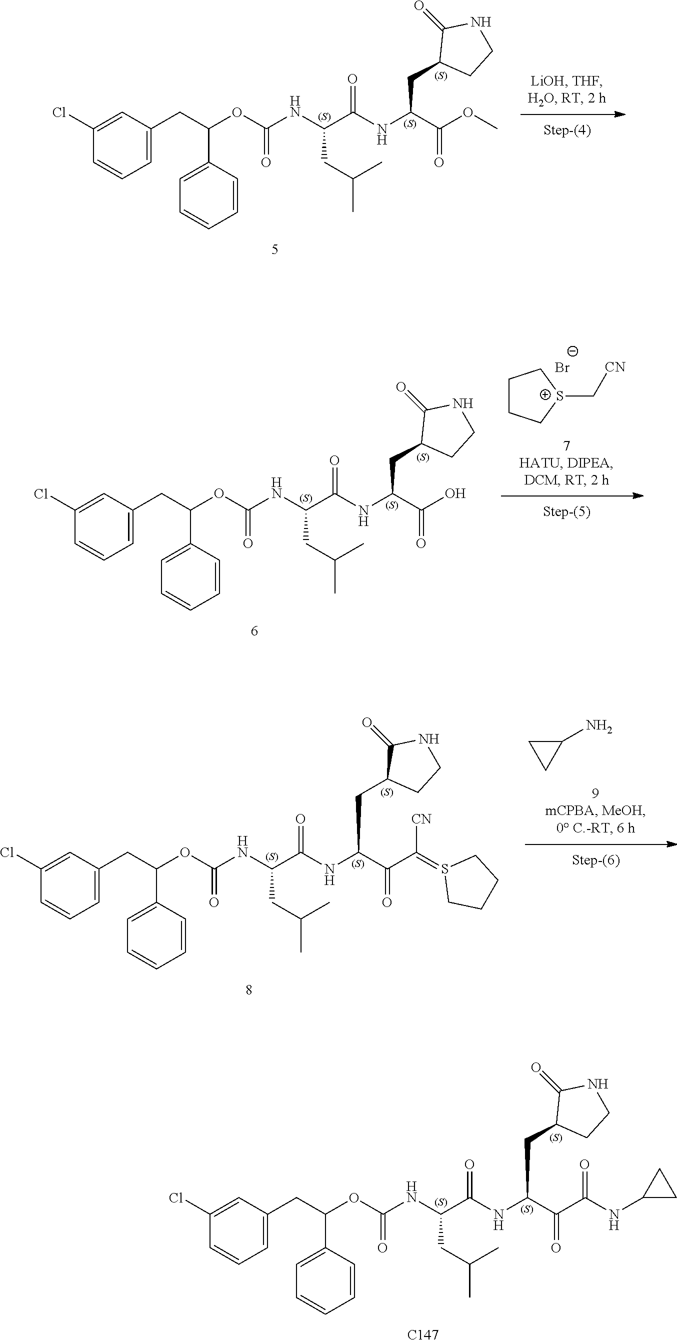

### Methyl ((2-(3-chlorophenyl)-1-phenylethoxy) carbonyl)-L-leucinate (3)

To a stirred solution of 2-(3-chlorophenyl)-1-phenylethan-1-ol (1) (4 g, 17.23 mmol) and methyl L-leucinate hydrochloride (2) (3.7 g, 25.85 mmol) in DCM (40 mL) was added pyridine (12 mL, 3 vol) followed by triphosgene (2.5 g, 8.61 mmol) at 0° C. and stirred at room temperature for 3 h. The progress of the reaction was monitored by TLC and LCMS. Reaction mixture was diluted with DCM and washed with 1N HCl (50 mL), organic layer was dried over sodium sulfate, filtered and evaporated under reduced pressure. The crude residue was purified by combi-flash, compound eluted at 10% ethyl acetate in pet ether to afford methyl ((2-(3-chlorophenyl)-1-phenylethoxy) carbonyl)-L-leucinate (3). TLC system: 10% Ethyl acetate in hexane Rf: 0.3 LCMS (ESI): m/z 426.43 [M+Na]⁺

### ((2-(3-Chlorophenyl)-1-phenylethoxy) carbonyl)-L-leucine (4)

To a stirred solution of methyl ((2-(3-chlorophenyl)-1-phenylethoxy)carbonyl)-L-leucinate (4) (2.2 g, 5.45 mmol) in THF (30 mL), water (15 mL) was added lithium hydroxide (671 mg, 16.37 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×50 mL), dried over sodium sulfate, concentrated under reduced pressure to afford ((2-(3-chlorophenyl)-1-phenylethoxy)carbonyl)-L-leucine (4). TLC system: 100% EtOAc Rf: 0.1 LCMS (ESI): m/z 412.41 [M+Na]⁺

### Methyl (2S)-2-((2S)-2-(((2-(3-chlorophenyl)-1-phenylethoxy) carbonyl) amino)-4-methylpentanamido)-3-((S)-2-oxopyrrolidin-3-yl) propanoate (5)

To a stirred solution of ((2-(3-chlorophenyl)-1-phenylethoxy)carbonyl)-L-leucine (4) (1.7 g, 4.36 mmol) DMF (20 mL) added EDC.HCl (1.2 g, 6.55 mmol), HOBt (0.88 g, 6.55 mmol), DIPEA (1.8 mL, 13.10 mmol) and methyl (S)-2-amino-3-((S)-2-oxopyrrolidin-3-yl)propanoate hydrochloride (amine fragment-2) (0.97 g, 5.24 mmol) at 0° C. simultaneously and stirred at room temperature for 16 h. Reaction mixture was quenched with ice water (80 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% methanol in dichloromethane to afford methyl (2S)-2-((2S)-2-(((2-(3-chlorophenyl)-1-phenylethoxy) carbonyl) amino)-4-methyl pentanamido)-3-((S)-2-oxopyrrolidin-3-yl) propanoate (5). TLC system: 10% Methanol in dichloromethane Rf: 0.4 LCMS (ESI): m/z 558.57 [M+H]⁺

### (2S)-2-((2S)-2-(((2-(3-chlorophenyl)-1-phenylethoxy) carbonyl) amino)-4-methylpentanamido)-3-((S)-2-oxopyrrolidin-3-yl) propanoic acid (6)

To a stirred solution of methyl (2S)-2-((2S)-2-(((2-(3-chlorophenyl)-1-phenylethoxy) carbonyl) amino)-4-methylpentanamido)-3-((S)-2-oxopyrrolidin-3-yl) propanoate (5) (1 g, 1.79 mmol) in THF (15 mL), water (5 mL) was added lithium hydroxide (220 mg, 5.37 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×50 mL), dried over sodium sulfate, concentrated under reduced pressure to afford (2S)-2-((2S)-2-(((2-(3-chlorophenyl)-1-phenylethoxy)carbonyl)amino)-4-methylpentanamido)-3-((S)-2-oxopyrrolidin-3-yl) propanoic acid (9). TLC system: 100% EtOAc Rf: 0.1 LCMS (ESI): m/z 544.52 [M+H]⁺

### 2-(3-Chlorophenyl)-1-phenylethyl ((S)-1-(((S)-4-cyano-3-oxo-1-((S)-2-oxopyrrolidin-3-yl)-4-(tetrahydro-1l4-thiophen-1-ylidene) butan-2-yl) amino)-4-methyl-1-oxopentan-2-yl)carbamate (8)

To a stirred solution of (2S)-2-((2S)-2-(((2-(3-chlorophenyl)-1-phenylethoxy) carbonyl) amino)-4-methylpentanamido)-3-((S)-2-oxopyrrolidin-3-yl)propanoic acid (6) (800 mg, 1.473 mmol) DCM (10 mL) added HATU (839 mg, 2.209 mmol), DIPEA (0.6 mL, 4.41 mmol) and 1-(cyanomethyl) tetrahydro-1H-thiophen-1-iumbromide (7) (455 mg, 2.209 mmol) at 0° C. simultaneously and stirred at room temperature for 2 h. Reaction mixture was diluted with ice water (30 mL), extracted with dichloromethane (2×30 mL), dried over sodium sulfate and evaporated under reduced pressure. The crude residue was purified by combi-flash NP, compound eluted at 5% methanol in dichloromethane to afford 2-(3-chlorophenyl)-1-phenylethyl ((S)-1-(((S)-4-cyano-3-oxo-1-((S)-2-oxopyrrolidin-3-yl)-4-(tetrahydro-1l4-thiophen-1-ylidene) butan-2-yl) amino)-4-methyl-1-oxopentan-2-yl) carbamate (8). TLC system: 10% Methanol in dichloromethane Rf: 0.5 LCMS (ESI): m/z 653.52 [M+H]⁺

### 2-(3-Chlorophenyl)-1-phenylethyl ((S)-1-(((S)-4-(cyclopropylamino)-3,4-dioxo-1-((S)-2-oxopyrrolidin-3-yl) butan-2-yl) amino)-4-methyl-1-oxopentan-2-yl) carbamate (Compound C147)

To a stirred solution of 2-(3-chlorophenyl)-1-phenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl) carbamate (8) (200 mg, 0.30 mmol) in methanol (3 mL) was added m-CPBA (105 mg, 0.61 mmol) at 0° C. and the reaction mixture stirred for 2 h at 0° C. and added cyclo propylamine (1 mL) and stirred at RT for 16 h. The progress of the reaction was monitored by TLC and LCMS. Reaction mixture was quenched with sat. NaHCO₃ solution (40 mL) and extracted with DCM (2×20 mL). Organic layer was washed with brine solution (30 mL), dried over Na₂SO₄ and concentrated to get crude compound. The crude compound was purified by prep HPLC to afford 2-(3-chlorophenyl)-1-phenylethyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C147). TLC system: 10% Methanol in dichloromethane Rf: 0.3 LCMS (ESI): m/z 611.2 (M+H)⁺

### Example 114: Synthesis of Compound C148

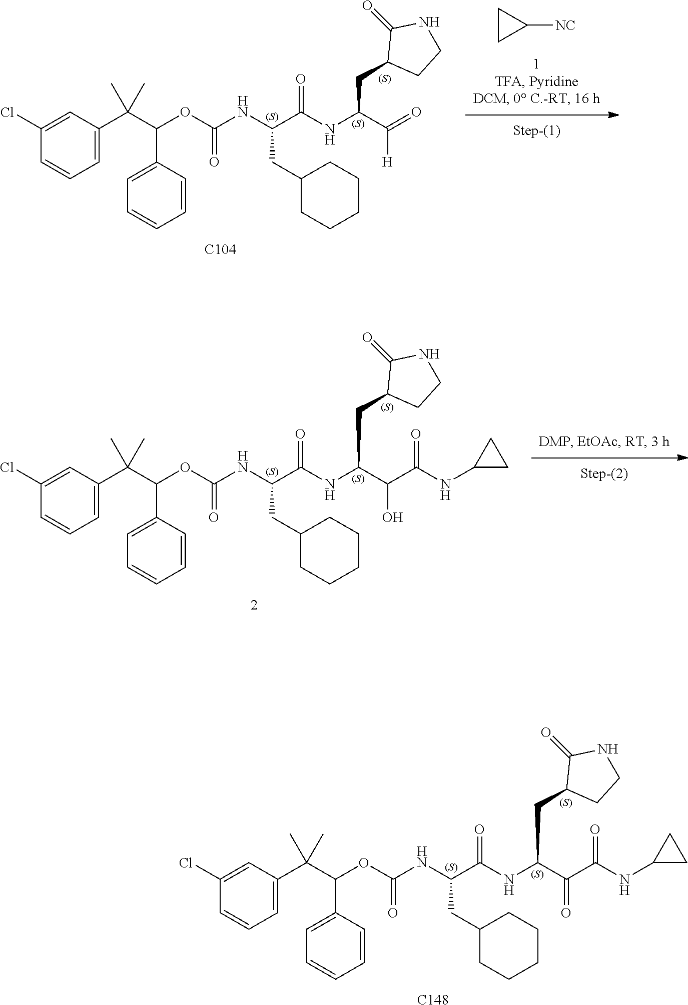

### 2-(3-Chlorophenyl)-2-methyl-1-phenylpropyl((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (2)

To a stirred solution of 2-(3-chlorophenyl)-2-methyl-1-phenylpropyl ((S)-3-cyclohexyl-1-oxo-1-(((S)-1-oxo-3-((S)-2-oxopyrrolidin-3-yl)propan-2-yl)amino)propan-2-yl)carbamate (Compound C104) (250 mg, 0.409 mmol) was dissolved in DCM (10 mL) added Pyridine (0.75 mL, 3 vol), isocyanocyclopropane (1) (41 mg, 0.614 mmol) sequentially at 0° C. and stirred for 10 min. To this was added TFA (0.06 mL, 819 mmol) at 0° C. and stirred at RT for 16 h. The progress of the reaction was monitored by TLC and LCMS. Reaction mixture was quenched with ice water (20 mL) and extracted with dichloromethane (2×15 mL). The organic layer was washed with 1N HCl (3×15 mL), brine solution (3×10 mL). The organic layer was dried over anhydrous Na₂SO₄ and evaporated under reduced pressure to afford crude 2-(3-Chlorophenyl)-2-methyl-1-phenyl propyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (2). TLC system: 5% Methanol/Dichloromethane Rf: 0.5 LCMS (ESI): m/z 681.6 [M+H]⁺

### 2-(3-Chlorophenyl)-2-methyl-1-phenylpropyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C148)

To a stirred solution of 2-(3-chlorophenyl)-2-methyl-1-phenylpropyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (2) (200 mg, 0.293 mmol) in EtOAc (10 mL) was added Dess-Martin periodinane (373 mg, 0.881 mmol) at 0° C. and stirred at RT for 3 h. The progress of the reaction was monitored by TLC and LCMS. Reaction mixture was filter through celite pad and washed with Ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford 2-(3-Chlorophenyl)-2-methyl-1-phenylpropyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C148). TLC system: 10% Methanol/Dichloromethane Rf: 0.4 LCMS (ESI): m/z 679.2 (M+H)⁺

### Example 115: Synthesis of Compound C149

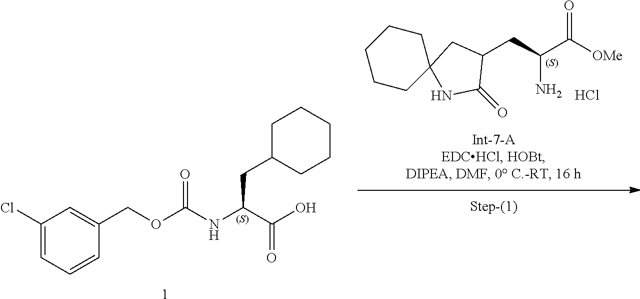

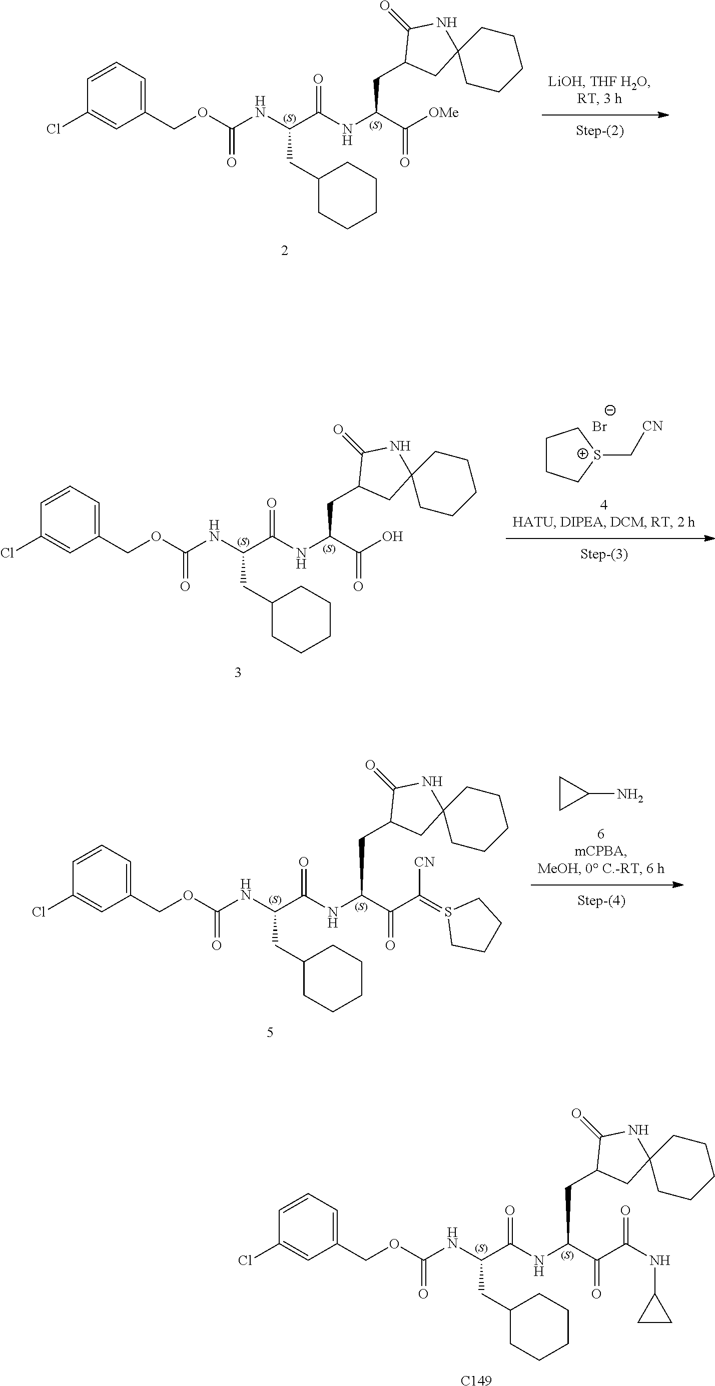

### Methyl (S)-2-((S)-2-((((3-chlorobenzyl)oxy)carbonyl)amino)-3-cyclohexylpropanamido)-3-((S)-2-oxo-1-azaspiro[4.5]decan-3-yl)propanoate (2)

To a stirred solution (S)-2-((((3-chlorobenzyl)oxy)carbonyl)amino)-3-cyclohexylpropanoic acid (Acid fragment) (1 g, 2.57 mmol) in DMF (20 mL) at 0° C. was added EDC.HCl (736 mg, 3.85 mmol), HOBT (519 mg, 3.85 mmol), DIPEA (1.34 mL, 7.71 mmol) and methyl 2-amino-3-(2-oxo-1-azaspiro[4.5]decan-3-yl)propanoate hydrochloride (Int-7A) (820 mg, 2.82 mmol) simultaneously and stirred at room temperature for 16 h. The progress of the reaction was monitored by TLC and LCMS. After 16 h, reaction mixture was quenched with ice water (50 mL), extracted with ethyl acetate (2×100 mL), the combined organic layer was dried over sodium sulfate and evaporated under reduced pressure. The crude residue was purified by silica gel column by eluting with 5% Methanol in DCM to afford methyl (S)-2-((S)-2-((((3-chlorobenzyl)oxy)carbonyl)amino)-3-cyclohexylpropanamido)-3-((S)-2-oxo-1-azaspiro[4.5]decan-3-yl)propanoate (2). TLC system: 5% Methanol/DCM R_{f}: 0.65 LCMS (ESI): m/z 576.66 (M+H)⁺

### (S)-2-((S)-2-((((3-chlorobenzyl)oxy)carbonyl)amino)-3-cyclohexylpropanamido)-3-((S)-2-oxo-1-azaspiro[4.5]decan-3-yl)propanoic acid (3)

To a stirred solution of methyl (S)-2-((S)-2-((((3-chlorobenzyl)oxy)carbonyl)amino)-3-cyclohexylpropanamido)-3-((S)-2-oxo-1-azaspiro[4.5]decan-3-yl)propanoate (2) (1 g, 1.73 mmol) in THF (7 mL), water (3 mL) was added lithium hydroxide (249 mg, 5.20 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×20 mL), dried over sodium sulfate, concentrated under reduced pressure to afford (S)-2-((S)-2-((((3-chlorobenzyl)oxy)carbonyl)amino)-3-cyclohexylpropanamido)-3-((S)-2-oxo-1-azaspiro[4.5]decan-3-yl)propanoic acid (3). TLC system: 10% MeOH in DCM Rf: 0.2 LCMS (ESI): m/z 562.47 [M+H]⁺

### 3-Chlorobenzyl ((S)-1-(((S)-4-cyano-3-oxo-1-((S)-2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (5)

To a stirred solution of (S)-2-((S)-2-((((3-chlorobenzyl)oxy)carbonyl)amino)-3-cyclohexylpropanamido)-3-((S)-2-oxo-1-azaspiro[4.5]decan-3-yl)propanoic acid (3) (800 mg, 1.42 mmol) in THF (8 mL) added HATU (798 mg, 2.135 mmol), DIPEA (0.74 mL, 4.270 mmol) and 1-(cyanomethyl)tetrahydro-1H-thiophen-1-ium (4) (461 mg, 2.135 mmol) at 0° C. simultaneously and stirred at room temperature for 2 h. Reaction mixture was diluted with ice water (50 mL), extracted with EtoAc (2×20 mL), dried over sodium sulfate and evaporated under reduced pressure. The crude residue was purified by combi-flash NP, compound eluted at 5% methanol in dichloromethane to afford 3-chlorobenzyl ((S)-1-(((S)-4-cyano-3-oxo-1-((S)-2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (5). TLC system: 10% Methanol in dichloromethane Rf: 0.4 LCMS (ESI): m/z 671.53 [M+H]⁺

### 3-Chlorobenzyl ((S)-3-cyclohexyl-1-(((S)-4-(cyclopropylamino)-3,4-dioxo-1-((S)-2-oxo-1-azaspiro[4.5]decan-3-yl)butan-2-yl)amino)-1-oxopropan-2-yl)carbamate (Compound C149)

To a stirred solution 3-chlorobenzyl ((S)-1-(((S)-4-cyano-3-oxo-1-((S)-2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (5) (300 mg, 0.447 mmol) in methanol (3 mL) was added m-CPBA (192 mg, 1.117 mmol) at 0° C. and the reaction mixture stirred for 2 h at 0° C. and added cyclopropanamine (6) (1.5 mL, 5 vol)) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with brine solution (30 mL), dried over Na₂SO₄ and concentrated to get crude compound. The crude compound was purified by prep-HPLC to afford 3-chlorobenzyl ((S)-3-cyclohexyl-1-(((S)-4-(cyclopropylamino)-3,4-dioxo-1-((S)-2-oxo-1-azaspiro[4.5]decan-3-yl)butan-2-yl)amino)-1-oxopropan-2-yl)carbamate (Compound C149). TLC system: 10% Methanol in dichloromethane Rf: 0.3 LCMS (ESI): m/z 629.2 [M+H]⁺

### Example 116: Synthesis of Compound C150

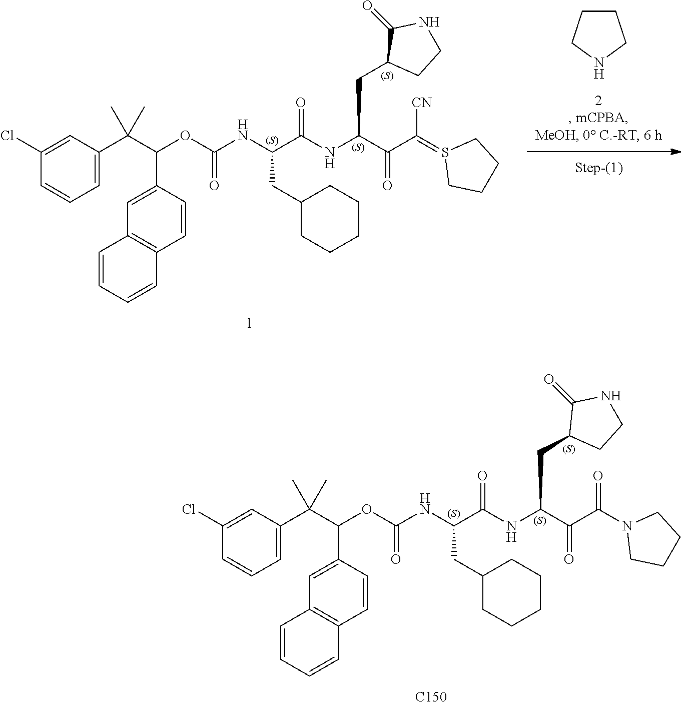

### 2-(3-Chlorophenyl)-2-methyl-1-(naphthalen-2-yl)propyl ((S)-3-cyclohexyl-1-(((S)-3,4-dioxo-1-((S)-2-oxopyrrolidin-3-yl)-4-(pyrrolidin-1-yl)butan-2-yl)amino)-1-oxopropan-2-yl)carbamate (Compound C150)

To a stirred solution of 2-(3-chlorophenyl)-2-methyl-1-(naphthalen-2-yl)propyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (1) (400 mg, 0.519 mmol) in methanol (2.5 mL) was added m-CPBA (223 mg, 1.29 mmol) at 0° C. and the reaction mixture stirred for 2 h at 0° C. and added pyrrolidine (0.738 mL, 5.1 mmol) and stirred at RT for 16 h. The progress of the reaction was monitored by TLC and LCMS. Reaction mixture was quenched with sat. NaHCO₃ solution (40 mL) and extracted with 10% methanol in DCM (2×15 mL). Organic layer was washed with brine solution (30 mL), dried over sodium sulfate and concentrated to get crude compound. The crude compound was purified by prep-HPLC to afford 2-(3-chlorophenyl)-2-methyl-1-(naphthalen-2-yl)propyl ((S)-3-cyclohexyl-1-(((S)-3,4-dioxo-1-((S)-2-oxopyrrolidin-3-yl)-4-(pyrrolidin-1-yl)butan-2-yl)amino)-1-oxopropan-2-yl)carbamate (Compound C150). TLC system: 10% Methanol in dichloromethane Rf: 0.3 LCMS (ESI): m/z 743.2 [M+H]+

### Example 117: Synthesis of Compound C151

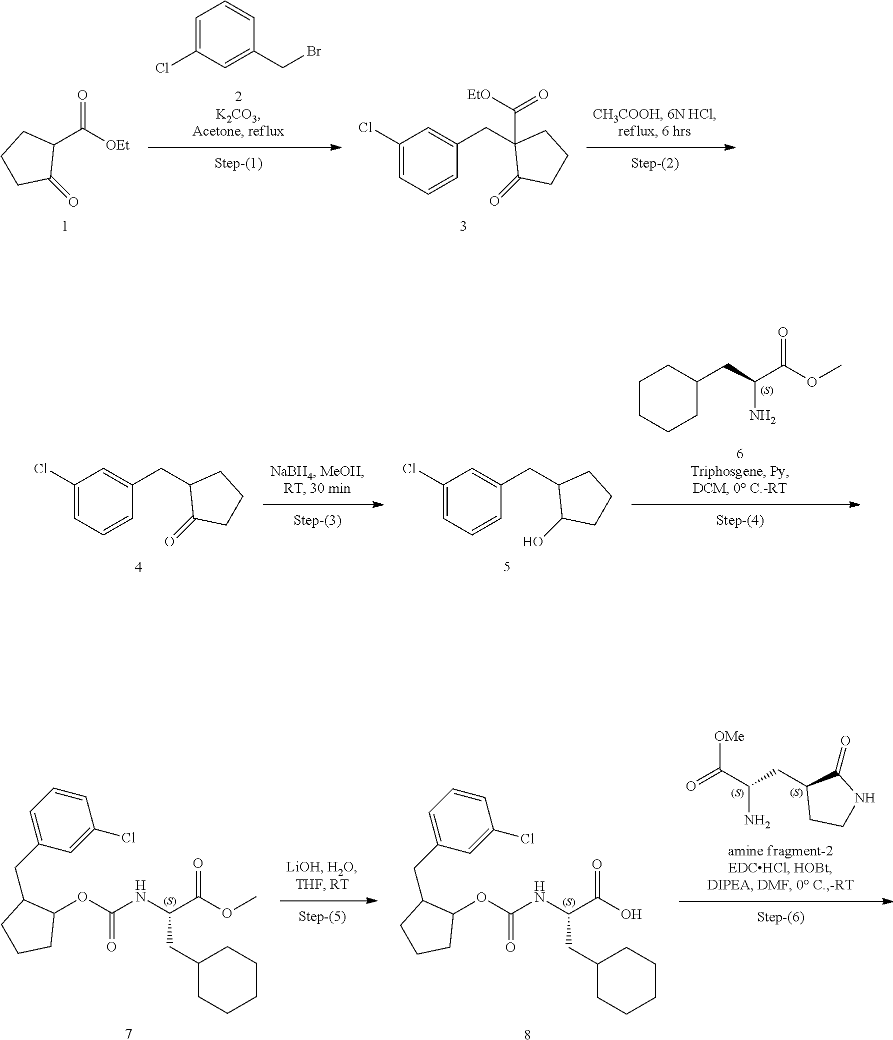

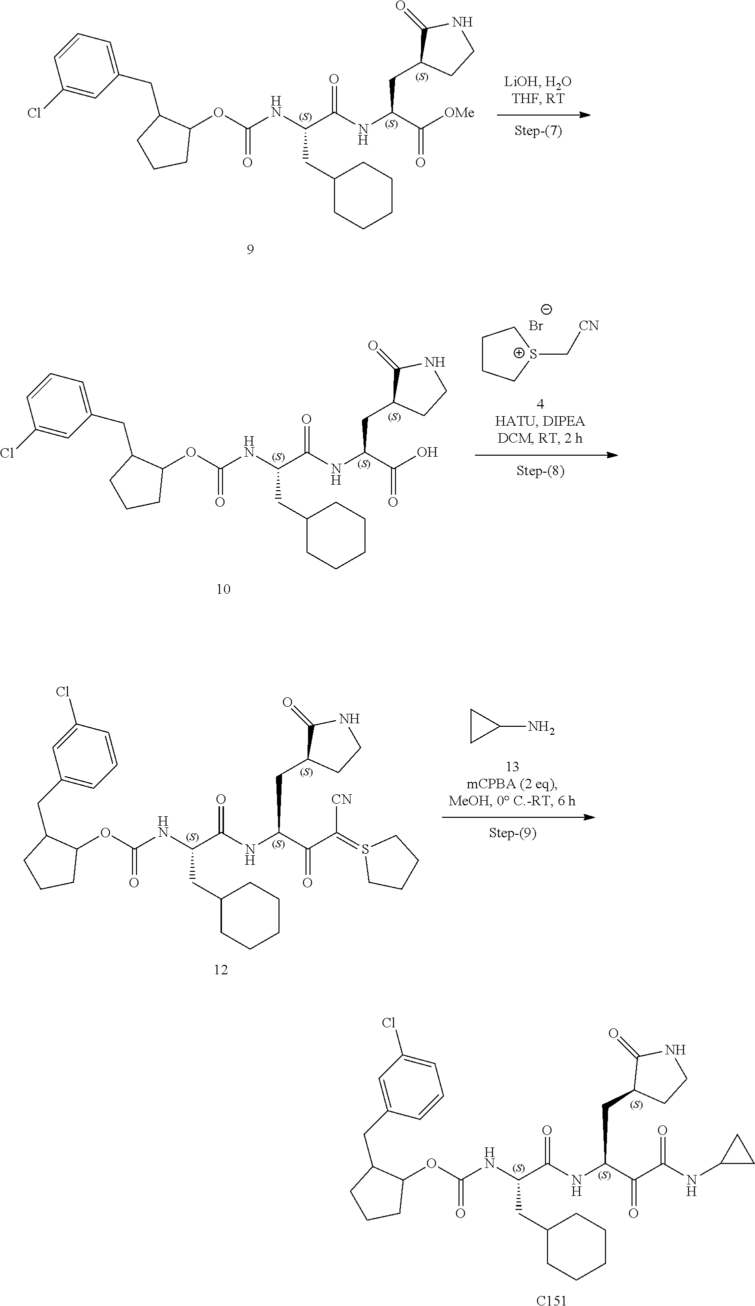

### Ethyl 1-(3-chlorobenzyl)-2-oxocyclopentane-1-carboxylate (3)

To a stirred solution of K₂CO₃ (17.6 g, 128.205 mmol) in Acetone (100 mL) was added ethyl 2-oxocyclopentane-1-carboxylate (1) (10 g, 64.102 mmol) at RT, followed by 1-(bromomethyl)-3-chlorobenzene (9.6 mL, 76.923 mmol) at RT and the reaction mixture was refluxed at 70° C. for 16 h. The progress of the reaction was monitored by TLC. Reaction mixture was cooled to RT and quenched with saturated NaHCO₃ (150 mL) then extracted with ethyl acetate (2×100 mL) then washed with the ethyl acetate layer with saturated NaHCO₃ (3×100 mL), dried over sodium sulfate and evaporated under reduced pressure. The crude residue was purified by silica gel column by eluting with 3% ethyl acetate in hexane to afford ethyl 1-(3-chlorobenzyl)-2-oxocyclopentane-1-carboxylate (3). TLC system: 5% Ethyl acetate in Hexane Rf: 0.5 LCMS (ESI): m/z=281.23 [M+H]⁺

### 2-(3-chlorobenzyl)cyclopentan-1-one (4)

To a stirred solution of ethyl 1-(3-chlorobenzyl)-2-oxocyclopentane-1-carboxylate (3) (15 g, 35.714 mmol) in glacial acetic acid (150 mL) was added 6N aq.HCl (75 mL) at RT and the reaction mixture was refluxed at 120° C. for 6 h. The progress of the reaction was monitored by TLC. Reaction mixture was cooled to RT and poured into ice-cold water (100 mL) and extracted with ethyl acetate (3×50 mL), dried over sodium sulfate and evaporated under reduced pressure. The crude residue was purified by silica gel column by eluting with 2% ethyl acetate in hexane to afford 2-(3-chlorobenzyl)cyclopentan-1-one (4). TLC system: 5% Ethyl acetate in Hexane Rf: 0.5 LCMS (ESI): m/z=209.09 [M+H]⁺

### 2-(3-chlorobenzyl)cyclopentan-1-ol (5)

To a stirred solution of 2-(3-chlorobenzyl)cyclopentan-1-one (4) (5 g, 24.038 mmol) in MeOH (50 mL) was added NaBH₄ (1.82 g, 48.076 mmol) at 0° C. Reaction mixture was allowed to stir at RT for 30 min. The progress of the reaction was monitored by TLC. Reaction mixture was distilled and quenched with 1N HCl extracted with ethyl acetate (2×50 mL), dried over sodium sulfate and evaporated under reduced pressure to afford the crude compound 2-(3-chlorobenzyl)cyclopentan-1-ol (5). TLC system: 5% Ethyl acetate in Hexane Rf: 0.2 LCMS (ESI): m/z=193.14 [M−OH]

### Methyl (2S)-2-((((2-(3-chlorobenzyl)cyclopentyl)oxy)carbonyl)amino)-3-cyclohexylpropanoate (7)

To a stirred solution of 2-(3-chlorobenzyl)cyclopentan-1-ol (5) (5 g, 23.809 mmol) in DCM (50 mL) was added pyridine (15 mL), Triphosgene (3.5 g, 11.904 mmol) at 0° C. slowly, followed by methyl (S)-2-amino-3-cyclohexylpropanoate (6) (5.3 g, 28.571 mmol) and stirred at RT for 16 h. The progress of the reaction was monitored by TLC. Reaction mixture was quenched with ice water (50 mL), concentrated the organic layer, again washed with 1N aq HCl solution, then extracted with DCM (2×50 mL), dried over sodium sulfate and evaporated under reduced pressure. The crude residue was purified by silica gel column by eluting with 4% ethyl acetate in hexane to afford methyl (2S)-2-((((2-(3-chlorobenzyl)cyclopentyl)oxy) carbonyl)amino)-3-cyclohexylpropanoate (7). TLC system: 20% Ethyl acetate in Hexane Rf: 0.5 LCMS (ESI): m/z=422.26 [M+H]+

### (2S)-2-((((2-(3-chlorobenzyl)cyclopentyl)oxy)carbonyl)amino)-3-cyclohexylpropanoic acid (8)

To a stirred solution of methyl (2S)-2-((((2-(3-chlorobenzyl)cyclopentyl)oxy) carbonyl)amino)-3-cyclohexylpropanoate (7) (3 g, 7.125 mmol) in THF (30 mL), DM water (10 mL), LiOH.H₂O (513 mg, 21.377 mmol) was added. Reaction mixture was stirred at RT for 2 h. The progress of the reaction was monitored by TLC. After consumption of starting material, the reaction mixture was concentrated and acidified with 1N aq. HCl, extracted with ethyl acetate (2×25 mL), dried over sodium sulfate and evaporated under reduced pressure to afford (2S)-2-((((2-(3-chlorobenzyl)cyclopentyl)oxy)carbonyl) amino)-3-cyclohexylpropanoic acid (8) which was used directly in the next step. TLC system: 5% Methanol in dichloromethane Rf: 0.2 LCMS (ESI): m/z=430.38 [M+Na]⁺

### Methyl (2S)-2-((2S)-2-((((2-(3-chlorobenzyl)cyclopentyl)oxy)carbonyl)amino)-3-cyclohexylpropanamido)-3-((S)-2-oxopyrrolidin-3-yl)propanoate (9)

To a stirred solution of (2S)-2-((((2-(3-chlorobenzyl)cyclopentyl)oxy)carbonyl) amino)-3-cyclohexylpropanoic acid (8) (2.2 g, 5.405 mmol), in DMF (20 mL) was added EDC.HCl (1.5 g, 8.108 mmol), HOBT (1.09 g, 8.108 mmol), DIPEA (2.9 mL, 16.216 mmol) and methyl (2S)-2-amino-3-(2-oxopyrrolidin-3-yl)propanoate (amine fragment-2) (1.2 g, 6.486 mmol) at 0° C. simultaneously and stirred at room temperature for 16 h. Reaction mixture was diluted with ice water (100 mL), extracted with ethyl acetate (2×50 mL), dried over sodium sulfate and evaporated under reduced pressure. The crude residue was purified by silica gel column by eluting with 5% methanol in dichloromethane to afford methyl (2S)-2-((2S)-2-((((2-(3-chlorobenzyl)cyclopentyl)oxy)carbonyl)amino)-3-cyclohexylpropanamido)-3-((S)-2-oxopyrrolidin-3-yl)propanoate (9). TLC system: 10% Methanol in dichloromethane Rf: 0.3 LCMS (ESI): m/z=576.61 [M+H]⁺

### (2S)-2-((2S)-2-((((2-(3-chlorobenzyl)cyclopentyl)oxy)carbonyl)amino)-3-cyclohexylpropanamido)-3-((S)-2-oxopyrrolidin-3-yl)propanoic acid (10)

To a stirred solution of methyl (2S)-2-((2S)-2-((((2-(3-chlorobenzyl)cyclopentyl)oxy)carbonyl) amino)-3-cyclohexylpropanamido)-3-((S)-2-oxopyrrolidin-3-yl)propanoate (9) (1 g, 1.739 mmol) in THF (10 mL), water (5 mL) was added lithium hydroxide (125 mg, 5.217 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-((2S)-2-((((2-(3-chlorobenzyl) cyclopentyl)oxy)carbonyl)amino)-3-cyclohexylpropanamido)-3-((S)-2-oxopyrrolidin-3-yl) propanoic acid (10). TLC system: 10% Methanol in dichloromethane Rf: 0.1 LCMS (ESI): m/z 562.47 [M+H]⁺

### 2-(3-Chlorobenzyl)cyclopentyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (12)

To a stirred solution of (2S)-2-((2S)-2-((((2-(3-chlorobenzyl) cyclopentyl)oxy)carbonyl) amino)-3-cyclohexylpropanamido)-3-((S)-2-oxopyrrolidin-3-yl) propanoic acid (10) (0.7 g, 1.247 mmol) DCM (10 mL) added HATU (0.71 g, 1.871 mmol), DIPEA (0.66 mL, 3.743 mmol) and 1-(cyanomethyl)tetrahydro-1H-thiophen-1-iumbromide (11) (239 mg, 1.871 mmol) at 0° C. simultaneously and stirred at room temperature for 2 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 2-(3-chlorobenzyl)cyclopentyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (12). TLC system: 10% Methanol in dichloromethane Rf: 0.4 LCMS (ESI): m/z 671.31 [M+H]⁺

### 2-(3-Chlorobenzyl)cyclopentyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C151)

To a stirred solution of 2-(3-chlorobenzyl)cyclopentyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (12) (250 mg, 0.373 mmol) in methanol (5 mL) was added mCPBA (160 mg, 0.932 mmol) at 0° C. and the reaction mixture stirred for 2 h at 0° C. and added cyclopropanamine (13) (25 mg, 0.447 mmol) and stirred at RT for 16 h. The progress of the reaction was monitored by TLC and LCMS. Reaction mixture was quenched with sat. NaHCO₃ solution (20 mL) and extracted with DCM (2×10 mL). Organic layer was washed with brine solution (20 mL), dried over Na₂SO₄ and concentrated to get crude compound. The crude compound was purified by prep HPLC to afford 2-(3-chlorobenzyl)cyclopentyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C151). TLC system: 10% Methanol in dichloromethane Rf: 0.3 LCMS (ESI): m/z 629.2 [M+H]⁺

### Example 118: Synthesis of Compound C152

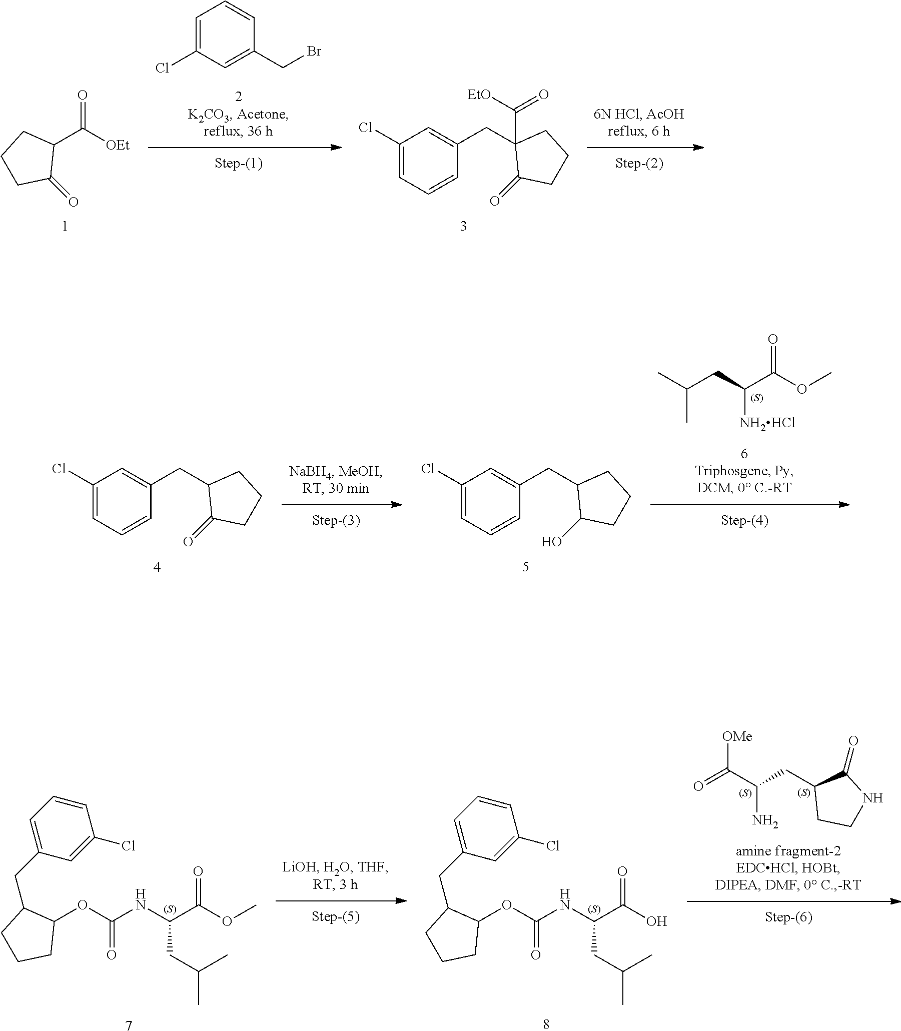

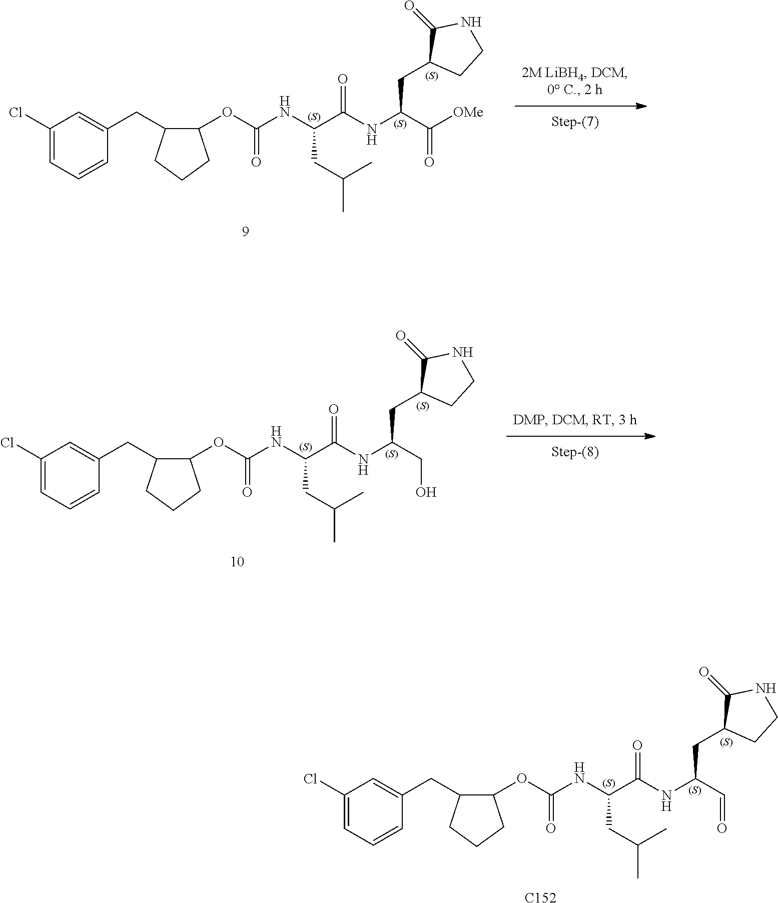

### Ethyl 1-(3-chlorobenzyl)-2-oxocyclopentane-1-carboxylate (3)

To a stirred solution of K₂CO₃ (17.6 g, 128.205 mmol) in acetone (100 mL) was added ethyl 2-oxocyclopentane-1-carboxylate (1) (10 g, 64.102 mmol) followed by 1-(bromo methyl)-3-chlorobenzene (9.6 mL, 76.923 mmol) at RT and the reaction mixture was refluxed at 70° C. for 36 h. The progress of the reaction was monitored by TLC. Reaction mixture was cooled to RT and quenched with saturated NaHCO₃ solution (150 mL) then extracted with ethyl acetate (2×100 mL). Organic layer was washed with saturated NaHCO₃ (3×100 mL), dried over sodium sulfate and evaporated under reduced pressure. The crude residue was purified by silica gel column by eluting with 3% ethyl acetate in hexane to afford ethyl 1-(3-chlorobenzyl)-2-oxocyclopentane-1-carboxylate (3). TLC system: 5% Ethyl acetate in hexane Rf: 0.5 LCMS (ESI): m/z=281.23 [M+H]⁺

### 2-(3-chlorobenzyl) cyclopentan-1-one (4)

To a stirred solution of ethyl 1-(3-chlorobenzyl)-2-oxocyclopentane-1-carboxylate (3) (15 g, 35.714 mmol) in glacial acetic acid (150 mL) was added 6N aq.HCl (75 mL) at RT and the reaction mixture was refluxed at 120° C. for 6 h. The progress of the reaction was monitored by TLC. Reaction mixture was cooled to RT and poured into ice-cold water (100 mL) and extracted with ethyl acetate (3×50 mL), dried over sodium sulfate and evaporated under reduced pressure. The crude residue was purified by silica gel column by eluting with 2% ethyl acetate in hexane to afford 2-(3-chlorobenzyl)cyclopentan-1-one (4). TLC system: 5% Ethyl acetate in hexane Rf: 0.5 LCMS (ESI): m/z=209.09 [M+H]⁺

### 2-(3-Chlorobenzyl) cyclopentan-1-ol (5)

To a stirred solution of 2-(3-chlorobenzyl) cyclopentan-1-one (4) (5 g, 24.038 mmol) in MeOH (50 mL) was added NaBH₄ (1.82 g, 48.076 mmol) at 0° C. Reaction mixture was allowed to RT and stirred for 30 min. The progress of the reaction was monitored by TLC. Reaction mixture was quenched with 1N HCl and evaporated to remove solvent then extracted with ethyl acetate (2×50 mL), dried over sodium sulfate, concentrated under reduced pressure to afford the crude compound 2-(3-chlorobenzyl)cyclopentan-1-ol (5). TLC system: 5% Ethyl acetate in hexane Rf: 0.2 LCMS (ESI): m/z=193.14 [M−OH]

### Methyl (((2-benzylcyclopentyl) oxy) carbonyl)-L-leucinate (6)

To a stirred solution of 2-(3-chlorobenzyl) cyclopentan-1-ol (5) (1.7 g, 8.095 mmol), methyl L-leucinate hydrochloride (5) (1.4 g, 9.714 mmol) in DCM (15 mL) was added pyridine (5.1 mL, 24.285 mmol) followed by triphosgene (1.19 g, 4.047 mmol) at 0° C. with portion wise and stirred at RT for 16 h. The progress of the reaction was monitored by TLC. Reaction mixture was quenched with 1N aq HCl (50 mL) and extracted with DCM (2×50 mL), dried over sodium sulfate and evaporated under reduced pressure. The crude residue was purified by silica gel column by eluting with 4% ethyl acetate in hexane to afford methyl (((2-(3-chlorobenzyl)cyclopentyl)oxy) carbonyl)-L-leucinate (7). TLC system: 20% Ethyl acetate in hexane Rf: 0.5 LCMS (ESI): m/z=382.38 [M+H]+

### (((2-(3-Chlorobenzyl)cyclopentyl)oxy)carbonyl)-L-leucine (8)

To a stirred solution of methyl (((2-(3-chlorobenzyl)cyclopentyl)oxy) carbonyl)-L-leucinate (7) (1.6 g, 4.199 mmol) in THF (20 mL), DM water (10 mL), LiOH.H₂O (302 mg, 12.598 mmol) was added. Reaction mixture was stirred at RT for 2 h. The progress of the reaction was monitored by TLC. After consumption of starting material, the reaction mixture was concentrated and acidified with 1N aq. HCl, extracted with ethyl acetate (2×50 mL), dried over sodium sulfate and evaporated under reduced pressure to afford (((2-(3-chlorobenzyl)cyclopentyl)oxy)carbonyl)-L-leucine (8) which was used directly in the next step. TLC system: 5% Methanol in dichloromethane Rf: 0.2 LCMS (ESI): m/z=368.22 [M+H]⁺

### Methyl (2S)-2-((2S)-2-((((2-(3-chlorobenzyl)cyclopentyl)oxy)carbonyl)amino)-4-methylpentanamido)-3-((S)-2-oxopyrrolidin-3-yl)propanoate (9)

To a stirred solution of (((2-(3-chlorobenzyl)cyclopentyl)oxy)carbonyl)-L-leucine (8) (1.4 g, 3.814 mmol), in DMF (10 mL) was added EDC.HCl (1.09 g, 5.722 mmol), HOBT (0.772 g, 5.722 mmol), DIPEA (2.1 mL, 18.018 mmol) and methyl (2S)-2-amino-3-(2-oxopyrrolidin-3-yl)propanoate (amine-fragment-2) (1.34 g, 15.422 mmol) at 0° C. simultaneously and stirred at room temperature for 16 h. Reaction mixture was diluted with ice water (100 mL), extracted with ethyl acetate (2×100 mL), dried over sodium sulfate and evaporated under reduced pressure. The crude residue was purified by silica gel column by eluting with 5% methanol in dichloromethane to afford methyl (2S)-2-((2S)-2-((((2-(3-chlorobenzyl)cyclopentyl)oxy) carbonyl)amino)-4-methylpentanamido)-3-((S)-2-oxopyrrolidin-3-yl)propanoate (9). TLC system: 10% Methanol in dichloromethane Rf: 0.3 LCMS (ESI): m/z=536.59 [M+H]⁺

### 2-(3-chlorobenzyl)cyclopentyl ((S)-1-(((S)-1-hydroxy-3-((S)-2-oxopyrrolidin-3-yl)propan-2-yl)amino)-4-methyl-1-oxopentan-2-yl)carbamate (10)

To a stirred solution of methyl (2S)-2-((2S)-2-((((2-(3-chlorobenzyl)cyclopentyl)oxy)carbonyl) amino)-4-methylpentanamido)-3-((S)-2-oxopyrrolidin-3-yl)propanoate (9) (0.8 g, 1.495 mmol) in Dichloromethane (10 mL), 2M LiBH₄ in THF (1.4 mL, 2.990 mmol) was added at 0° C. and stirred for 2 h The progress of the reaction was monitored by TLC and LCMS. the reaction mixture was quenched with saturated NH₄Cl solution and extracted with Dichloromethane (2×50 mL), filtrate was concentrated under reduced 2-(3-chlorobenzyl)cyclopentyl((S)-1-(((S)-1-hydroxy-3-((S)-2-oxopyrrolidin-3-yl)propan-2-yl) amino)-4-methyl-1-oxopentan-2-yl)carbamate (10). TLC system: 10% Methanol in dichloromethane Rf: 0.4 LCMS (ESI): m/z=508.38 [M+H]⁺

### 2-(3-chlorobenzyl)cyclopentyl ((S)-4-methyl-1-oxo-1-(((S)-1-oxo-3-((S)-2-oxopyrrolidin-3-yl)propan-2-yl)amino)pentan-2-yl)carbamate (Compound C152)

To a stirred solution of 2-(3-chlorobenzyl)cyclopentyl((S)-1-(((S)-1-hydroxy-3-((S)-2-oxopyrrolidin-3-yl)propan-2-yl) amino)-4-methyl-1-oxopentan-2-yl)carbamate (10) (0.1 g, 0.197 mmol) was dissolved in Ethyl acetate (10 mL) was added Dess-Martin periodinane (0.167 g, 0.394 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude compound. The crude compound was purified by Prep. HPLC to afford 2-(3-chlorobenzyl)cyclopentyl ((S)-4-methyl-1-oxo-1-(((S)-1-oxo-3-((S)-2-oxopyrrolidin-3-yl)propan-2-yl)amino)pentan-2-yl)carbamate (Compound C152). TLC system: 10% Methanol in dichloromethane Rf: 0.5 LCMS (ESI): m/z=506.1 [M+H]⁺

### Example 119: Synthesis of Compound C153

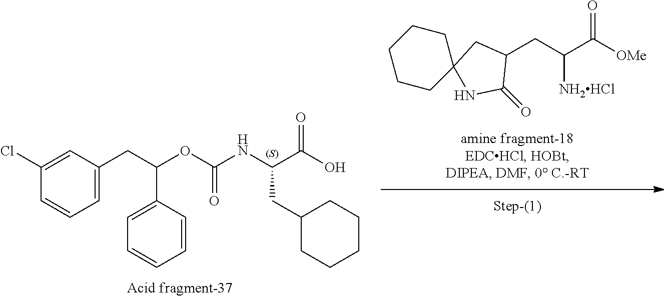

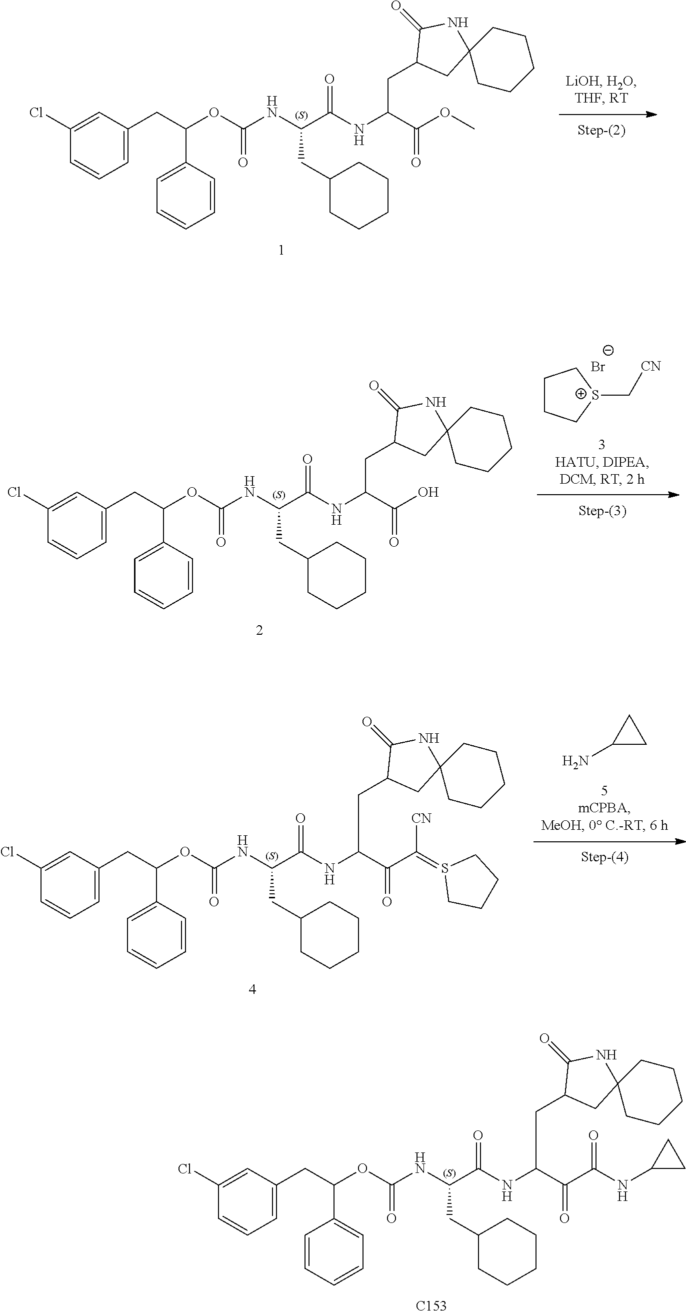

### Methyl 2-((2S)-2-(((2-(3-chlorophenyl)-1-phenylethoxy)carbonyl)amino)-3-cyclohexylpropanamido)-3-(2-oxo-1-azaspiro[4.5]decan-3-yl)propanoate (1)

To a stirred solution of (2S)-2-(((2-(3-chlorophenyl)-1-phenylethoxy)carbonyl)amino)-3-cyclohexylpropanoic acid (1) (Acid fragment-37) (1.0 g, 2.33 mmol) in DMF (15 mL) was added EDC.HCl (0.667 g, 3.49 mmol), HOBt (0.47 g, 3.49 mmol), DIPEA (1.3 mL, 6.99 mmol) and methyl-2-amino-3-(2-oxo-1-azaspiro[4.5]decan-3-yl)propanoate hydrochloride (Amine fragment-18) (0.68 g, 2.33 mmol) at 0° C., and stirred at room temperature for 16 h. Reaction mixture was quenched with ice water (250 mL) and extracted with ethyl acetate (2×100 mL). Combined the organic layer and washed with brine solution (2×200 mL), dried over sodium sulfate and evaporated under reduced pressure. The crude residue was purified by combi-flash NP, compound eluted at 10% methanol in dichloromethane to afford methyl 2-((2S)-2-(((2-(3-chlorophenyl)-1-phenylethoxy)carbonyl)amino)-3-cyclohexylpropanamido)-3-(2-oxo-1-azaspiro[4.5]decan-3-yl)propanoate (1). TLC system: 10% Methanol in dichloromethane Rf: 0.4 LCMS (ESI): m/z 666.50 [M+H]⁺

### 2-((2S)-2-(((2-(3-Chlorophenyl)-1-phenylethoxy)carbonyl)amino)-3-cyclohexylpropanamido)-3-(2-oxo-1-azaspiro[4.5]decan-3-yl)propanoic acid (2)

To a stirred solution of methyl 2-((2S)-2-(((2-(3-chlorophenyl)-1-phenylethoxy)carbonyl)amino)-3-cyclohexylpropanamido)-3-(2-oxo-1-azaspiro[4.5]decan-3-yl)propanoate (1) (0.9 g, 1.35 mmol) in THF (6 mL), water (6 mL) was added lithium hydroxide (170 mg, 4.06 mmol) at RT and stirred at room temperature for 2 h. The progress of the reaction was monitored by TLC and LCMS. Excess of THF was distilled under reduced pressure, crude compound acidified with aq. 1N HCl solution up to pH˜2 and extracted with DCM (2×50 mL), dried over sodium sulfate, concentrated under reduced pressure to afford 24(2S)-2-(((2-(3-chlorophenyl)-1-phenylethoxy)carbonyl)amino)-3-cyclohexylpropanamido)-3-(2-oxo-1-azaspiro[4.5]decan-3-yl)propanoic acid (2). TLC system: 10% Methanol in DCM Rf: 0.4 LCMS (ESI): m/z 652.49 [M+H]⁺

### 2-(3-Chlorophenyl)-1-phenylethyl ((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)carbamate (4)

To a stirred solution of 2-((2S)-2-(((2-(3-chlorophenyl)-1-phenylethoxy)carbonyl)amino)-3-cyclohexylpropanamido)-3-(2-oxo-1-azaspiro[4.5]decan-3-yl)propanoic acid (2) (800 mg, 1.27 mmol, in DCM (12 mL) was added HATU (700 mg, 1.83 mmol), DIPEA (0.7 mL, 3.66 mmol) and 1-(cyanomethyl)tetrahydro-1H-thiophen-1-ium (3) (305 mg, 2147 mmol) at 0° C., and stirred at room temperature for 2 h. Reaction mixture was diluted with ice water (50 mL), extracted with DCM (2×50 mL). The combined organic layers were washed with water (1×50 mL), brine solution (1×50 mL), dried over sodium sulfate and evaporated under vacuum. The crude residue was purified by combi-flash NP, compound eluted at 5% methanol in dichloromethane to afford 2-(3-chlorophenyl)-1-phenylethyl ((2S)-1-((4-cyano-3-oxo-1-(2-oxo-1-azaspiro[4.5]decan-3-yl)-4-(tetrahydro-1λ⁴-thiophen-1-ylidene)butan-2-yl)amino)-3-cyclohexyl-1-oxopropan-2-yl)carbamate (4). TLC system: 10% Methanol in dichloromethane Rf: 0.5 LCMS (ESI): m/z 761.43 [M+H]⁺

### 2-(3-Chlorophenyl)-1-phenylethyl ((2S)-3-cyclohexyl-1-((4-(cyclopropylamino)-3,4-dioxo-1-(2-oxo-1-azaspiro[4.5]decan-3-yl)butan-2-yl)amino)-1-oxopropan-2-yl)carbamate (C153)

To a stirred solution 2-(3-chlorophenyl)-1-phenylethyl ((2S)-1-((4-cyano-3-oxo-1-(2-oxo-1-azaspiro[4.5]decan-3-yl)-4-(tetrahydro-1λ⁴-thiophen-1-ylidene)butan-2-yl)amino)-3-cyclohexyl-1-oxopropan-2-yl)carbamate (4) (200 mg, 0.263 mmol) in methanol (3 mL) was added m-CPBA (68 mg, 0.394 mmol) at 0° C. and the reaction mixture was stirred for 2 h at 0° C. and then added cyclopropylamine (0.03 mL, 0.526 mmol) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with water (1×20 mL) and brine solution (1×30 mL), dried over Na₂SO₄ and concentrated to get crude compound. The crude compound was purified by prep-HPLC to afford 2-(3-chlorophenyl)-1-phenylethyl((2S)-3-cyclohexyl-1-((4-(cyclopropylamino)-3,4-dioxo-1-(2-oxo-1-azaspiro[4.5]decan-3-yl)butan-2-yl)amino)-1-oxopropan-2-yl)carbamate (C153). TLC system: 10% Methanol in dichloromethane Rf: 0.5 LCMS (ESI): m/z 719.2 [M+H]⁺

### Example 120: Synthesis of Compound C154

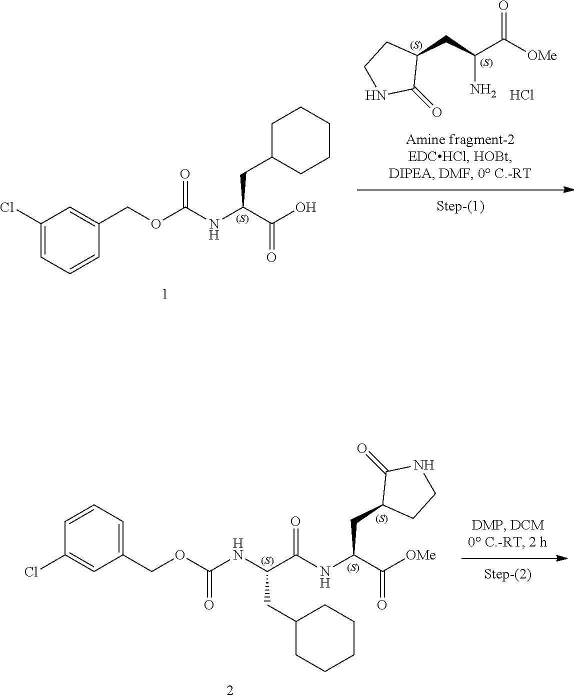

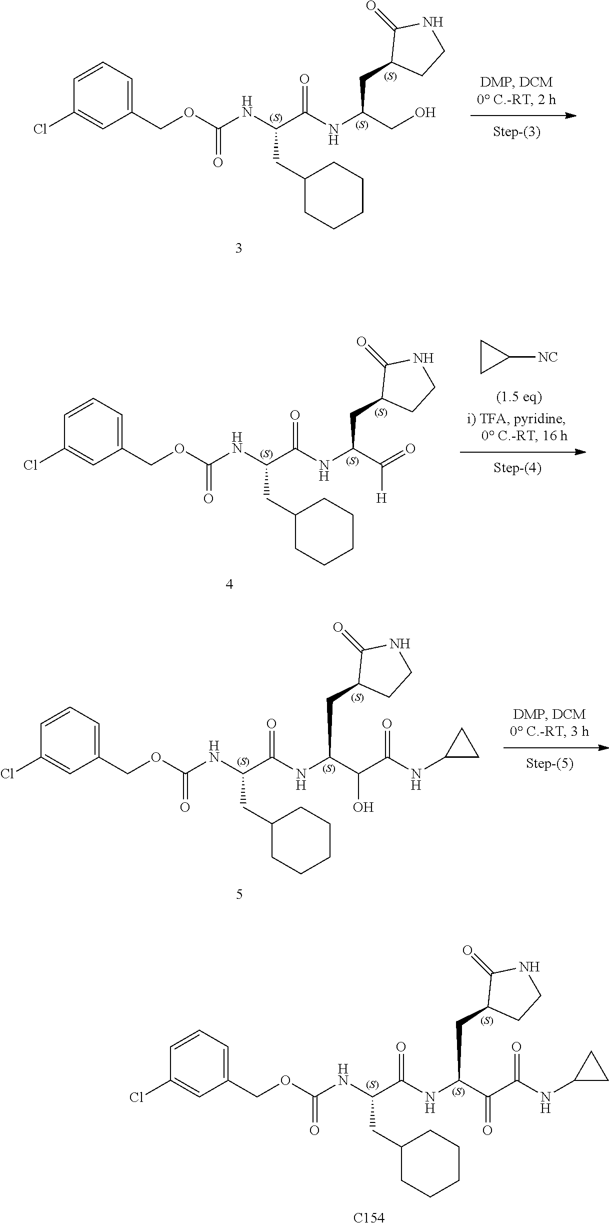

### Methyl (S)-2-((S)-2-((((3-chlorobenzyl)oxy)carbonyl)amino)-3-cyclohexylpropanamido)-3-((S)-2-oxopyrrolidin-3-yl)propanoate (2)

To a stirred solution of (S)-2-((((3-chlorobenzyl)oxy)carbonyl)amino)-3-cyclohexylpropanoic acid (1) (5.0 g, 14.7 mmol) in DMF (50 mL) was added EDC.HCl (4.2 g, 22.11 mmol), HOBt (2.98 g, 22.11 mmol), DIPEA (8.15 mL, 44.22 mmol) and methyl (S)-2-amino-3-((S)-2-oxopyrrolidin-3-yl)propanoate hydrochloride (amine fragment-2) (3.92 g, 17.69 mmol) at 0° C., and stirred at room temperature for 16 h. Reaction mixture was quenched with ice water (250 mL) and extracted with ethyl acetate (2×100 mL). Combined the organic layer and washed with brine solution (2×200 mL), dried over sodium sulfate and evaporated under reduced pressure. The crude residue was purified by combi-flash NP, compound eluted at 10% methanol in dichloromethane to afford methyl (S)-2-((S)-2-((((3-chlorobenzyl)oxy)carbonyl)amino)-3-cyclohexylpropanamido)-3-((S)-2-oxopyrrolidin-3-yl)propanoate (2). TLC system: 10% Methanol in dichloromethane Rf: 0.6 LCMS (ESI): m/z 508.47 [M+H]⁺

### 3-Chlorobenzyl ((S)-3-cyclohexyl-1-(((S)-1-hydroxy-3-((S)-2-oxopyrrolidin-3-yl)propan-2-yl)amino)-1-oxopropan-2-yl)carbamate (3)

To a stirred solution of methyl (S)-2-((S)-2-((((3-chlorobenzyl)oxy)carbonyl)amino)-3-cyclohexylpropanamido)-3-((S)-2-oxopyrrolidin-3-yl)propanoate (2) (4.0 g, 7.88 mmol) in DCM (200 mL) at 0° C. was added 2M LiBH₄ in THF (7.8 mL, 1.57 mmol) then reaction mixture was stirred for 1 h at room temperature. Reaction mixture was quenched with sat. ammonium chloride solution (200 mL) and extracted with DCM (2×200 mL). Organic layer was washed with brine solution, dried over Na₂SO₄ and concentrated to get crude to afford pure 3-chlorobenzyl ((S)-3-cyclohexyl-1-(((S)-1-hydroxy-3-((S)-2-oxopyrrolidin-3-yl)propan-2-yl)amino)-1-oxopropan-2-yl)carbamate (3). TLC system: 5% Methanol in DCM Rf: 0.4 LCMS (ESI): m/z 480.37[M+H]⁺

### 3-Chlorobenzyl ((S)-3-cyclohexyl-1-oxo-1-(((S)-1-oxo-3-((S)-2-oxopyrrolidin-3-yl)propan-2-yl)amino)propan-2-yl)carbamate (4)

To a stirred solution of 3-chlorobenzyl ((S)-3-cyclohexyl-1-(((S)-1-hydroxy-3-((S)-2-oxopyrrolidin-3-yl)propan-2-yl)amino)-1-oxopropan-2-yl)carbamate (3) (2.0 g, 4.16 mmol) in DCM (50 mL) was added Dess-Martin periodinane (5.29 g, 12.49 mmol) at 0° C. and stirred at RT for 3 h. The progress of the reaction was monitored by TLC and LCMS. Reaction mixture was filter through celite pad and washed with DCM (50 mL) and filtrate was washed with hypo solution (3×20 mL) followed by saturated NaHCO₃ solution (3×20 mL). Organic layer was dried over anhydrous Na₂SO₄, filtered and concentrated to get crude, residue was purified by combi-flash NP, compound eluted at 10% methanol in dichloromethane afford to 3-chlorobenzyl ((S)-3-cyclohexyl-1-oxo-1-(((S)-1-oxo-3-((S)-2-oxopyrrolidin-3-yl)propan-2-yl)amino)propan-2-yl)carbamate (4). TLC system: 5% Methanol in dichloromethane Rf: 0.3 LCMS (ESI): m/z 478.1 [M+H]⁺

### 3-Chlorobenzyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (5)

To a stirred solution of 3-chlorobenzyl ((S)-3-cyclohexyl-1-oxo-1-(((S)-1-oxo-3-((S)-2-oxopyrrolidin-3-yl)propan-2-yl)amino)propan-2-yl)carbamate (4) (600 mg, 1.25 mmol) and isocyanocyclopropane (126.41 mg, 1.88 mmol) was dissolved in DCM (60 mL), then added pyridine (3 mL), at 0° C. and stirred for 10 min. To this solution was added TFA (0.3 mL, 2.51 mmol) at 0° C. and stirred at RT for 16 h. The progress of the reaction was monitored by TLC and LCMS. Reaction mixture was quenched with ice water (20 mL) and extracted with dichloromethane (2×15 mL). The organic layer was washed with 1N HCl (3×15 mL), brine solution (3×10 mL). The organic layer was dried over anhydrous Na₂SO₄ and evaporated under reduced pressure to afford crude 3-chlorobenzyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (5). TLC system: 10% Methanol in dichloromethane Rf: 0.5 LCMS (ESI): m/z 563.2 [M+H]⁺

### 3-Chlorobenzyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (0154)

To a stirred solution of 3-chlorobenzyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (5) (450 mg, 0.799 mmol) was added Dess-Martin periodinane (677.79 mg, 1.59 mmol) at 0° C. and stirred at RT for 3 h. The progress of the reaction was monitored by TLC and LCMS. Reaction mixture was filter through celite pad and washed with DCM (50 mL) and filtrate was washed with hypo solution (3×20 mL) followed by saturated NaHCO₃ solution (3×20 mL). Organic layer was dried over anhydrous Na₂SO₄, filtered and concentrated to get crude, residue was purified by combi-flash NP, compound eluted at 10% methanol in dichloromethane afford to 3-chlorobenzyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (C154). TLC system: 10% Methanol in dichloromethane Rf: 0.4 LCMS (ESI): m/z 561.1 [M+H]⁺

### Example 121: Synthesis of Compound C155 and Compound C180

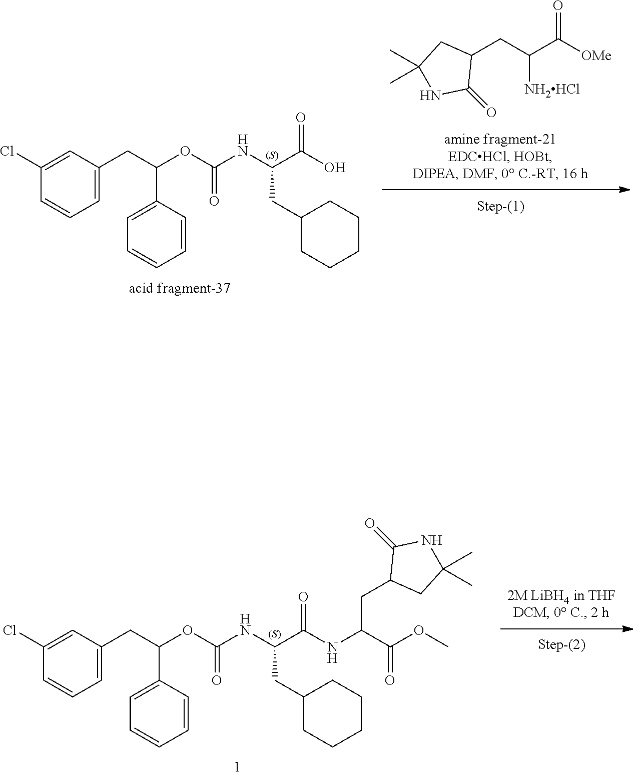

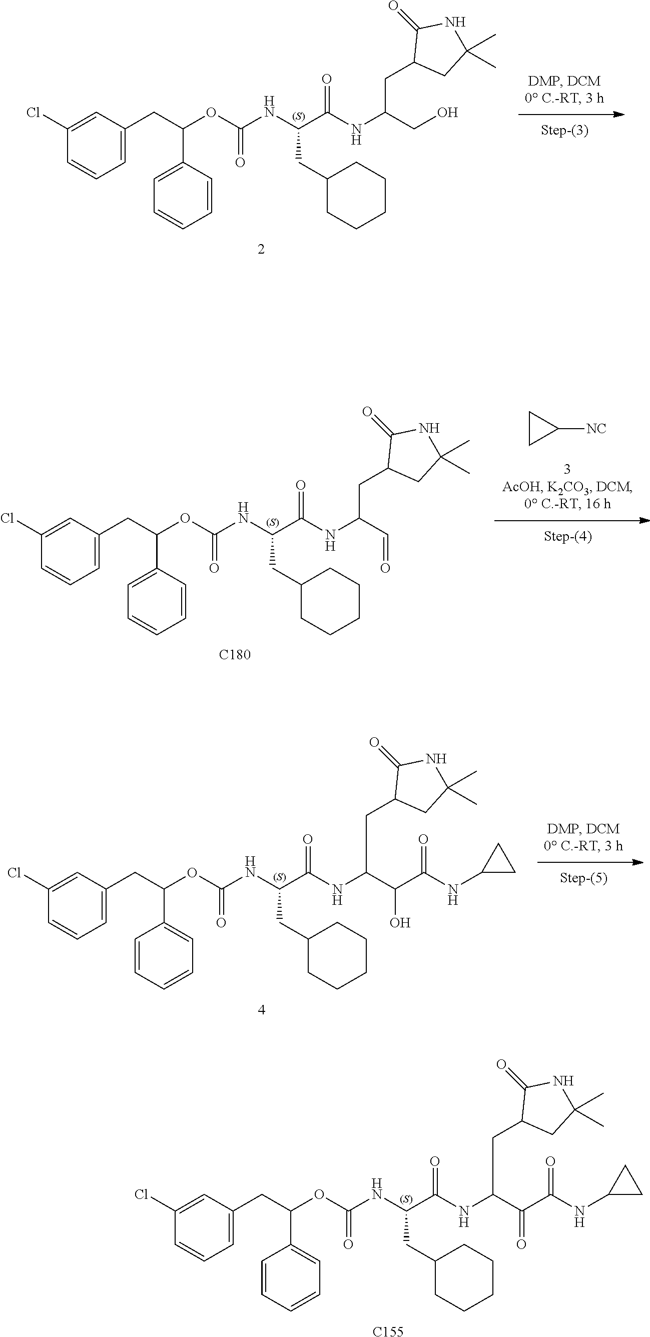

### Methyl 2-((2S)-2-(((2-(3-chlorophenyl)-1-phenylethoxy) carbonyl) amino)-3-cyclohexylpropanamido)-3-(5,5-dimethyl-2-oxopyrrolidin-3-yl) propanoate (1)

To a stirred solution of (2S)-2-(((2-(3-chlorophenyl)-1-phenylethoxy)carbonyl)amino)-3-cyclohexylpropanoic acid (acid fragment-37) (1 g, 2.33 mmol) DMF (10 mL) added EDC.HCl (667 mg, 3.49 mmol), HOBt (471 mg, 3.49 mmol), DIPEA (1 mL, 6.99 mmol) and methyl 2-amino-3-(5,5-dimethyl-2-oxopyrrolidin-3-yl)propanoate hydrochloride (amine fragment-21) (699 mg, 2.79 mmol) at 0° C. simultaneously and stirred at room temperature for 16 h. Reaction mixture was quenched with ice water (80 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% methanol in dichloromethane to afford methyl 2-((2S)-2-(((2-(3-chlorophenyl)-1-phenylethoxy) carbonyl) amino)-3-cyclohexyl propanamido)-3-(5,5-dimethyl-2-oxopyrrolidin-3-yl) propanoate (1). TLC system: 10% Methanol in dichloromethane Rf: 0.4 LCMS (ESI): m/z 626.48 [M+H]⁺

### 2-(3-Chlorophenyl)-1-phenylethyl ((2S)-3-cyclohexyl-1-((1-(5,5-dimethyl-2-oxopyrrolidin-3-yl)-3-hydroxypropan-2-yl) amino)-1-oxopropan-2-yl) carbamate (2)

To a stirred solution of methyl 2-((2S)-2-(((2-(3-chlorophenyl)-1-phenylethoxy) carbonyl)amino)-3-cyclohexylpropanamido)-3-(5,5-dimethyl-2-oxopyrrolidin-3-yl)propanoate (1) (920 mg, 1.47 mmol) in DCM (10 mL) was added 2M LiBH₄ in THF (1.4 mL, 2.94 mmol) at 0° C. and the reaction mixture stirred for 2 h at 0° C. The progress of the reaction was monitored by TLC and LCMS. Reaction mixture was quenched with sat. ammonium chloride solution (20 mL) and extracted with DCM (2×20 mL). Organic layer was washed with brine solution (30 mL), dried over Na₂SO₄ and concentrated to afford 2-(3-chlorophenyl)-1-phenylethyl ((2S)-3-cyclohexyl-1-((1-(5,5-dimethyl-2-oxopyrrolidin-3-yl)-3-hydroxypropan-2-yl)amino)-1-oxo propan-2-yl) carbamate (2). TLC system: 10% Methanol in dichloromethane Rf: 0.2 LCMS (ESI): m/z 598.40 [M+H]⁺

### 2-(3-Chlorophenyl)-1-phenylethyl ((2S)-3-cyclohexyl-1-((1-(5,5-dimethyl-2-oxopyrrolidin-3-yl)-3-oxopropan-2-yl) amino)-1-oxopropan-2-yl) carbamate (C180)

To a stirred solution of 2-(3-chlorophenyl)-1-phenylethyl ((2S)-3-cyclohexyl-1-((1-(5,5-dimethyl-2-oxopyrrolidin-3-yl)-3-hydroxypropan-2-yl)amino)-1-oxopropan-2-yl)carbamate (2) (150 mg, 0.25 mmol) was dissolved in dichloromethane (5 mL) was added Dess-Martin periodinane (213 mg, 0.50 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford 2-(3-chlorophenyl)-1-phenylethyl ((2S)-3-cyclohexyl-1-((1-(5,5-dimethyl-2-oxopyrrolidin-3-yl)-3-oxopropan-2-yl) amino)-1-oxopropan-2-yl) carbamate (C180). TLC system: 10% Methanol in dichloromethane Rf: 0.5 LCMS (ESI): m/z 596.52 (M+H)⁺

### 2-(3-Chlorophenyl)-1-phenylethyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (4)

To a stirred solution of 2-(3-chlorophenyl)-1-phenylethyl ((2S)-3-cyclohexyl-1-((1-(5,5-dimethyl-2-oxopyrrolidin-3-yl)-3-oxopropan-2-yl)amino)-1-oxopropan-2-yl)carbamate (C180) (300 mg, 0.50 mmol) in ethyl acetate (10 mL) was added acetic acid (1 mL) was added isocyanocyclopropane (3) (50 mg, 0.75 mmol) at 0° C. and stirred at RT for 16 h. The progress of the reaction was monitored by TLC and LCMS. After 16 h, the reaction mixture was directly concentrated to get the crude: this residue was dissolved in methanol (5 mL), water (2 mL) and added potassium carbonate (104 mg, 0.75 mmol) at RT and stirred at room temperature for 16 h. The progress of the reaction was monitored by TLC and LCMS. Reaction mixture completely distilled under reduced pressure, crude compound acidified with aq. 1N HCl solution up to pH˜3 and extracted with ethyl acetate (2×30 mL), dried over sodium sulfate, concentrated under reduce pressure to afford crude residue. This residue was triturated with di ethyl ether, (30 mL) to afford 2-(3-chlorophenyl)-1-phenylethyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (4). TLC system: 10% Methanol in dichloromethane Rf: 0.3 LCMS (ESI): m/z 681.56 [M+H]⁺

### 2-(3-Chlorophenyl)-1-phenylethyl ((2S)-3-cyclohexyl-1-((4-(cyclopropylamino)-1-(5,5-dimethyl-2-oxopyrrolidin-3-yl)-3,4-dioxobutan-2-yl) amino)-1-oxopropan-2-yl) carbamate (C155)

To a stirred solution of 2-(3-chlorophenyl)-1-phenylethyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (4) (280 mg, 0.41 mmol) was dissolved in dichloromethane (10 mL) was added Dess-Martin periodinane (349 mg, 0.82 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by combi-flash NP, compound eluted at 6% methanol in dichloromethane to afford 2-(3-chlorophenyl)-1-phenylethyl ((2S)-3-cyclohexyl-1-((4-(cyclopropylamino)-1-(5,5-dimethyl-2-oxo pyrrolidin-3-yl)-3,4-dioxobutan-2-yl) amino)-1-oxopropan-2-yl) carbamate (C155). TLC system: 10% Methanol in dichloromethane Rf: 0.5 LCMS (ESI): m/z 679.2 (M+H)⁺

### Example 122: Synthesis of Compound C156 and Compound C167

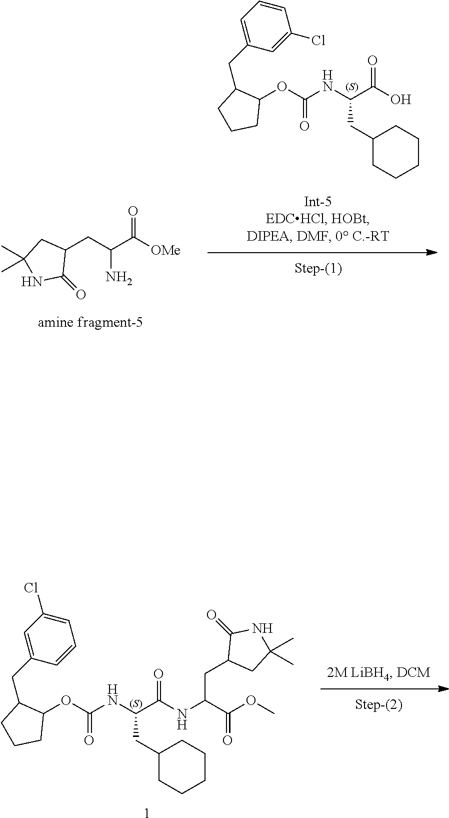

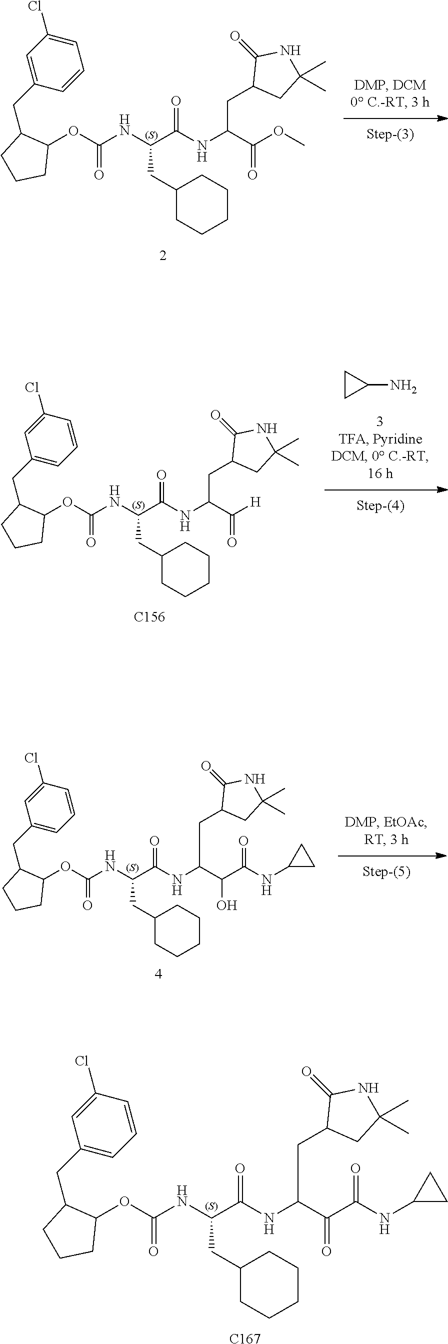

### Methyl 2-((2S)-2-((((2-(3-chlorobenzyl)cyclopentyl)oxy)carbonyl)amino)-3-cyclohexylpropanamido)-3-(5,5-dimethyl-2-oxopyrrolidin-3-yl)propanoate (1)

To a stirred solution of (2S)-2-((((2-(3-chlorobenzyl)cyclopentyl)oxy)carbonyl)amino)-3-cyclohexylpropanoic acid (Int-5) (1.5 g, 3.68 mmol) in DMF at 0° C. was added EDC.HCl (718 mg, 3.68 mmol), HOBT (490 mg, 3.62 mmol), DIPEA (4.5 mL, 3 Vol.) and methyl 2-amino-3-(5,5-dimethyl-2-oxopyrrolidin-3-yl)propanoate hydrochloride (amine fragment 5) (737 mg, 11.05 mmol) simultaneously and stirred at room temperature for 16 h. The progress of the reaction was monitored by TLC and LCMS. After 16 h, reaction mixture was quenched with ice water (20 mL), extracted with ethyl acetate (2×30 mL), the combined organic layer was dried over sodium sulfate and evaporated under reduced pressure. The crude residue was purified by silica gel column by eluting with 50% ethyl acetate in pet ether to afford methyl 24(2S)-2-((((2-(3-chlorobenzyl)cyclopentyl)oxy)carbonyl)amino)-3-cyclohexylpropanamido)-3-(5,5-dimethyl-2-oxopyrrolidin-3-yl)propanoate. TLC system: 5% Methanol in DCM R_{f}: 0.5 LCMS (ESI): m/z 604.5 (M+H)⁺

### 2-((2S)-2-((((2-(3-Chlorobenzyl)cyclopentyl)oxy)carbonyl)amino)-3-cyclohexylpropanamido)-3-(5,5-dimethyl-2-oxopyrrolidin-3-yl)propanoic acid (2)

To a stirred solution of methyl 2-((2S)-2-((((2-(3-chlorobenzyl)cyclopentyl)oxy)carbonyl)amino)-3-cyclohexylpropanamido)-3-(5,5-dimethyl-2-oxopyrrolidin-3-yl)propanoate (1) (1 g, 1.65 mmol) in DCM (20 mL) was added 2M LiBH₄ in THF (2.48 mL, 4.96 mmol) at 0° C. and the reaction mixture was stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford 2-((2S)-2-((((2-(3-chlorobenzyl)cyclopentyl)oxy)carbonyl)amino)-3-cyclohexylpropanamido)-3-(5,5-dimethyl-2-oxopyrrolidin-3-yl)propanoic acid (2). TLC system: 5% MeOH in DCM R_{f} 0.3 LCMS (ESI): m/z 576.5 (M+H)⁺

### 2-(3-Chlorobenzyl)cyclopentyl ((2S)-3-cyclohexyl-1-((1-(5,5-dimethyl-2-oxopyrrolidin-3-yl)-3-oxopropan-2-yl)amino)-1-oxopropan-2-yl)carbamate (C156)

To a stirred solution of 2-((2S)-2-((((2-(3-chlorobenzyl)cyclopentyl)oxy)carbonyl)amino)-3-cyclohexylpropanamido)-3-(5,5-dimethyl-2-oxopyrrolidin-3-yl)propanoic acid (2) (200 mg, 0.33 mmol) in ethyl acetate (10 mL) was added Dess-Martin periodinane (422 mg, 0.99 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 2-(3-chlorobenzyl)cyclopentyl ((2S)-3-cyclohexyl-1-((1-(5,5-dimethyl-2-oxopyrrolidin-3-yl)-3-oxopropan-2-yl)amino)-1-oxopropan-2-yl)carbamate (C156). TLC system: 5% Methanol in DCM R_{f}: 0.4 LCMS (ESI): m/z 574.2 (M+H)⁺

### 2-(3-Chlorobenzyl)cyclopentyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (4)

To a stirred solution of 2-(3-chlorobenzyl)cyclopentyl ((2S)-3-cyclohexyl-1-((1-(5,5-dimethyl-2-oxopyrrolidin-3-yl)-3-oxopropan-2-yl)amino)-1-oxopropan-2-yl)carbamate (C156) (350 mg, 0.60 mmol) was dissolved in DCM (20 mL), added Pyridine (1 mL, 3 vol), isocyanocyclopropane (3) (61 mg, 0.91 mmol) sequentially at 0° C. and stirred for 10 min. To this was added TFA (0.13 mL, 1.21 mmol) at 0° C. and stirred at RT for 16 h. The progress of the reaction was monitored by TLC and LCMS. Reaction mixture was quenched with ice water (20 mL) and extracted with dichloromethane (2×15 mL). The organic layer was washed with 1N HCl (3×15 mL), brine solution (3×10 mL). The organic layer was dried over anhydrous Na₂SO₄ and evaporated under reduced pressure to afford crude 2-(3-chlorobenzyl)cyclopentyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (4). TLC system: 5% Methanol/Dichloromethane Rf: 0.5 LCMS (ESI): m/z 659.5 [M+H]⁺

### 2-(3-Chlorobenzyl)cyclopentyl ((2S)-3-cyclohexyl-1-((4-(cyclopropylamino)-1-(5,5-dimethyl-2-oxopyrrolidin-3-yl)-3,4-dioxobutan-2-yl)amino)-1-oxopropan-2-yl)carbamate (C167)

To a stirred solution of 2-(3-chlorobenzyl)cyclopentyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (4) (200 mg, 0.30 mmol) in EtOAc (10 mL) was added Dess-Martin periodinane (386 mg, 0.91 mmol) at 0° C. and stirred at RT for 3 h. The progress of the reaction was monitored by TLC and LCMS. Reaction mixture was filter through celite pad and washed with Ethyl acetate (25 mL) and filtrate was washed with hypo solution (3×10 mL) followed by saturated NaHCO₃ solution (3×10 mL). Organic layer was dried over anhydrous Na₂SO₄, filtered and concentrated to get crude residue. The crude compound was purified by prep HPLC to afford 2-(3-chlorobenzyl)cyclopentyl ((2S)-3-cyclohexyl-1-((4-(cyclopropylamino)-1-(5,5-dimethyl-2-oxopyrrolidin-3-yl)-3,4-dioxobutan-2-yl)amino)-1-oxopropan-2-yl)carbamate (C167). TLC system: 10% Methanol/Dichloromethane Rf: 0.4 LCMS (ESI): m/z 657.2 (M+H)⁺

### Example 123: Synthesis of Compounds C174 and C157

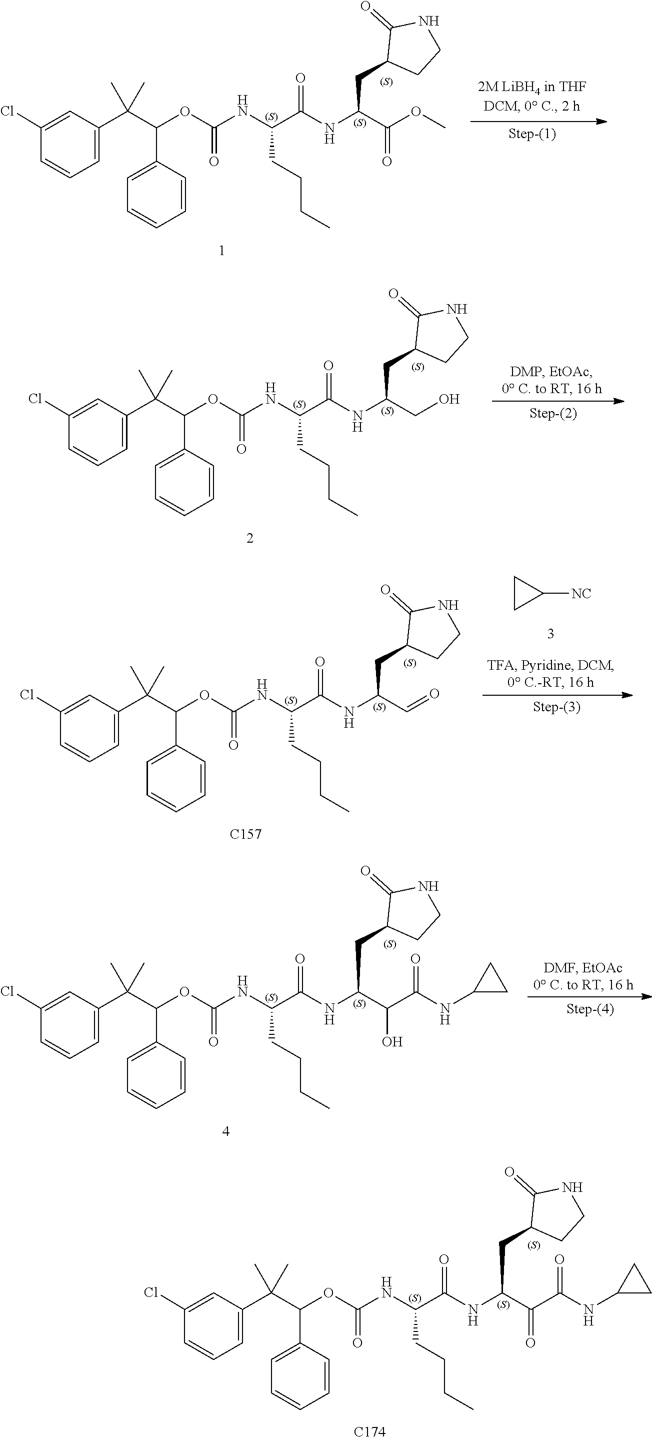

### 2-(3-Chlorophenyl)-2-methyl-1-phenylpropyl ((S)-1-(((S)-1-hydroxy-3-((S)-2-oxopyrrolidin-3-yl) propan-2-yl) amino)-1-oxohexan-2-yl) carbamate (2)

To a stirred solution of methyl (2S)-2-((2S)-2-(((2-(3-chlorophenyl)-2-methyl-1-phenylpropoxy)carbonyl)amino)hexanamido)-3-((S)-2-oxopyrrolidin-3-yl)propanoate (1) (600 mg, 1.02 mmol) in DCM (10 mL) was added 2M LiBH₄ in THF (1 mL, 1.53 mmol) at 0° C. and the reaction mixture stirred for 2 h at 0° C. The progress of the reaction was monitored by TLC and LCMS. Reaction mixture was quenched with sat. ammonium chloride solution (20 mL) and extracted with DCM (2×20 mL). Organic layer was washed with brine solution (30 mL), dried over Na₂SO₄ and concentrated to afford 2-(3-chlorophenyl)-2-methyl-1-phenylpropyl ((S)-1-(((S)-1-hydroxy-3-((S)-2-oxo pyrrolidin-3-yl) propan-2-yl)amino)-1-oxohexan-2-yl) carbamate (2). TLC system: 10% Ethyl acetate in hexane Rf: 0.3 LCMS (ESI): m/z 558.52 [M+H]⁺

### 2-(3-Chlorophenyl)-2-methyl-1-phenylpropyl ((S)-1-oxo-1-(((S)-1-oxo-3-((S)-2-oxopyrrolidin-3-yl) propan-2-yl) amino) hexan-2-yl) carbamate (C157)

To a stirred solution of 2-(3-chlorophenyl)-2-methyl-1-phenylpropyl ((S)-1-(((S)-1-hydroxy-3-((S)-2-oxopyrrolidin-3-yl)propan-2-yl)amino)-1-oxohexan-2-yl)carbamate (2) (100 mg, 0.15 mmol) was dissolved in ethyl acetate (5 mL) was added Dess-Martin periodinane (519 mg, 1.02 mmol) at 0° C. and stirred at RT for 16 h. Reaction mixture was filtered through celite bed and washed with ethyl acetate (30 mL). Filtrate was washed with sat. Hypo solution (3×20 mL) followed by sat. NaHCO₃ solution (3×20 mL). Organic layer was dried over anhydrous Na₂SO₄, filtered and concentrated to get crude compound. The crude residue was purified by combi-flash NP, compound eluted at 5% methanol in dichloromethane to afford 2-(3-chlorophenyl)-2-methyl-1-phenylpropyl ((S)-1-oxo-1-(((S)-1-oxo-3-((S)-2-oxopyrrolidin-3-yl) propan-2-yl) amino) hexan-2-yl) carbamate (C157). TLC system: 10% Methanol in dichloromethane Rf: 0.4 LCMS (ESI): m/z 558.52 [M+H]⁺

### 2-(3-Chlorophenyl)-2-methyl-1-phenylpropyl ((2S)-1-(((2S)-4-(cyclopropylamino)-3-hydroxy-4-oxo-1-((S)-2-oxopyrrolidin-3-yl) butan-2-yl) amino)-1-oxohexan-2-yl) carbamate (4)

To a stirred solution of 2-(3-chlorophenyl)-2-methyl-1-phenylpropyl ((S)-1-oxo-1-(((S)-1-oxo-3-((S)-2-oxopyrrolidin-3-yl)propan-2-yl)amino)hexan-2-yl)carbamate (C157) (250 mg, 0.45 mmol) in DCM (10 mL) was added isocyanocyclopropane (3) (45 mg, 0.67 mmol), pyridine (71 mg, 0.90 mmol) followed by TFA (102 mg, 0.90 mmol) at 0° C. and stirred at RT for 16 h. The progress of the reaction was monitored by TLC and LCMS. After 16 h, the reaction mixture was diluted with dichloromethane and washed with 1N HCl solution (15 mL) followed by brine (15 mL), dried over sodium sulfate, concentrated under reduce pressure to afford crude residue. This residue was triturated with di ethyl ether (30 mL) to afford 2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (4). TLC system: 10% Methanol in dichloromethane Rf: 0.4 LCMS (ESI): m/z 641.55 [M+H]⁺

### 2-(3-Chlorophenyl)-2-methyl-1-phenylpropyl ((S)-1-(((S)-4-(cyclopropylamino)-3,4-dioxo-1-((S)-2-oxopyrrolidin-3-yl) butan-2-yl) amino)-1-oxohexan-2-yl) carbamate (C174)

To a stirred solution of 2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-1-oxohexan-2-yl)carbamate (4) (210 mg, 0.32 mmol) was dissolved in ethyl acetate (10 mL) was added Dess-Martin periodinane (417 mg, 0.98 mmol) at 0° C. and stirred at RT for 16 h. Reaction mixture was filtered through celite bed and washed with ethyl acetate (20 mL). Filtrate was washed with sat. Hypo solution (3×20 mL) followed by sat. NaHCO₃ solution (3×20 mL). Organic layer was dried over anhydrous Na₂SO₄, filtered and concentrated to get crude compound. The crude residue was purified by combi-flash NP, compound eluted at 8% methanol in dichloromethane to afford 2-(3-chlorophenyl)-2-methyl-1-phenylpropyl ((S)-1-(((S)-4-(cyclopropylamino)-3,4-dioxo-1-((S)-2-oxopyrrolidin-3-yl)butan-2-yl)amino)-1-oxohexan-2-yl)carbamate (C174). TLC system: 10% Methanol in dichloromethane Rf: 0.5 LCMS (ESI): m/z 639.2 [M+H]⁺

### Example 124: Synthesis of Compound C158

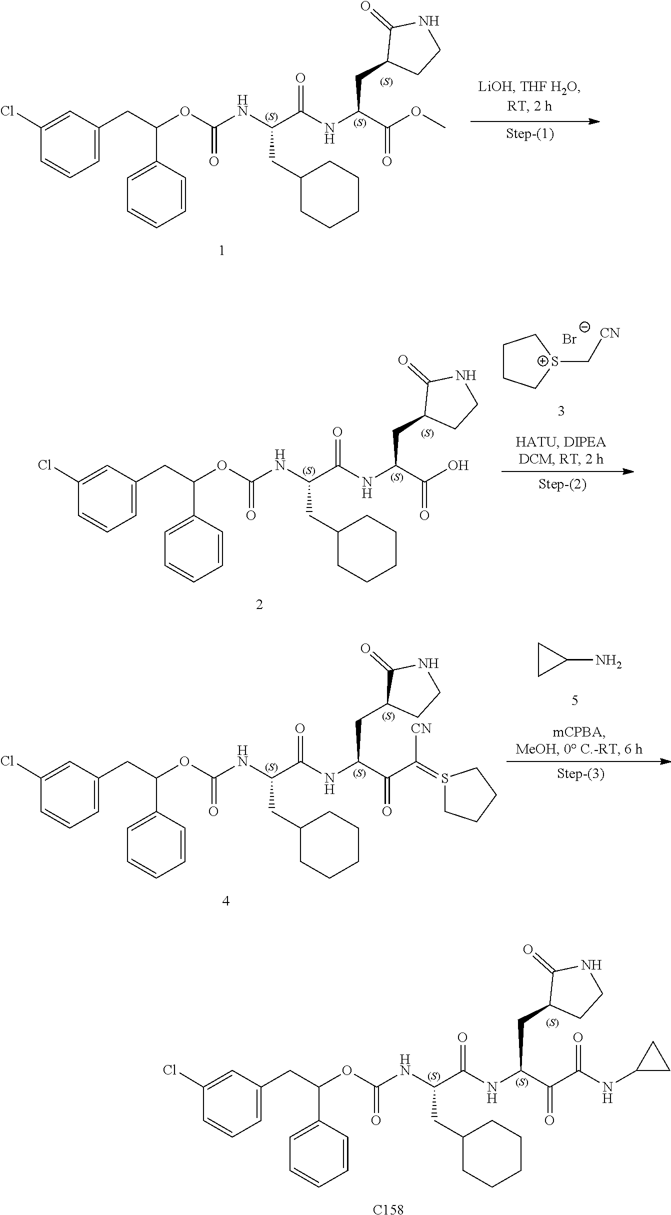

### (2S)-2-((2S)-2-(((2-(3-chlorophenyl)-1-phenylethoxy) carbonyl) amino)-3-cyclohexylpropanamido)-3-((S)-2-oxopyrrolidin-3-yl) propanoic acid (2)

To a stirred solution of methyl (2S)-2-((2S)-2-(((2-(3-chlorophenyl)-1-phenylethoxy)carbonyl)amino)-3-cyclohexylpropanamido)-3-((S)-2-oxopyrrolidin-3-yl) propanoate (1) (0.7 g, 1.17 mmol) in THF (5 mL), methanol (5 mL) and water (3 mL) was added lithium hydroxide (144 mg, 3.51 mmol) at RT and stirred at room temperature for 2 h. The progress of the reaction was monitored by TLC and LCMS. Reaction mixture completely distilled under reduced pressure, crude compound acidified with aq. 1N HCl solution up to pH˜3 and extracted with ethyl acetate (2×30 mL), dried over sodium sulfate, concentrated under reduced pressure to afford (2S)-2-(((2-(3-chlorophenyl)-1-phenylethoxy) carbonyl) amino)-3-cyclohexylpropanoic acid (2). TLC system: 100% EtOAc Rf: 0.1 LCMS (ESI): m/z 584.60 [M+H]⁺

### 2-(3-Chlorophenyl)-1-phenylethyl ((S)-1-(((S)-4-cyano-3-oxo-1-((S)-2-oxopyrrolidin-3-yl)-4-(tetrahydro-1l4-thiophen-1-ylidene) butan-2-yl) amino)-3-cyclohexyl-1-oxopropan-2-yl) carbamate (4)

To a stirred solution of (2S)-2-((2S)-2-(((2-(3-chlorophenyl)-1-phenylethoxy) carbonyl) amino)-3-cyclohexylpropanamido)-3-((S)-2-oxopyrrolidin-3-yl) propanoic acid (2) (420 mg, 0.72 mmol) DCM (10 mL) added HATU (410 mg, 1.08 mmol), DIPEA (0.3 mL, 2.16 mmol) and 1-(cyanomethyl)tetrahydro-1H-thiophen-1-iumbromide (3) (222 mg, 1.08 mmol) at 0° C. simultaneously and stirred at room temperature for 2 h. Reaction mixture was diluted with ice water (50 mL), extracted with dichloromethane (2×30 mL), dried over sodium sulfate and evaporated under reduced pressure. The crude residue was purified by combi-flash NP, compound eluted at 5% methanol in dichloromethane to afford 1-(3-chlorobenzyl) cyclopropyl ((S)-1-(((S)-4-cyano-3-oxo-1-((S)-2-oxopyrrolidin-3-yl)-4-(tetrahydro-1l4-thiophen-1-ylidene) butan-2-yl) amino)-4-methyl-1-oxopentan-2-yl) carbamate (4). TLC system: 10% Methanol in dichloromethane Rf: 0.5 LCMS (ESI): m/z 693.53 (M+H)⁺

### 2-(3-Chlorophenyl)-1-phenylethyl ((S)-3-cyclohexyl-1-(((S)-4-(cyclopropylamino)-3,4-dioxo-1-((S)-2-oxopyrrolidin-3-yl) butan-2-yl) amino)-1-oxopropan-2-yl) carbonate (C158)

To a stirred solution of 2-(3-chlorophenyl)-1-phenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl) carbamate (4) (200 mg, 0.28 mmol) in methanol (4 mL) was added m-CPBA (99 mg, 0.57 mmol) at 0° C. and the reaction mixture stirred for 2 h at 0° C. and added cyclopropanamine (1 mL) and stirred at RT for 16 h. The progress of the reaction was monitored by TLC and LCMS. Reaction mixture was diluted with dichloromethane and washed with sat. NaHCO₃ solution (2×40 mL) and extracted with DCM (2×15 mL). Organic layer was washed with brine solution (30 mL), dried over Na₂SO₄ and concentrated to get crude compound. The crude compound was purified by prep HPLC to afford 2-(3-chlorophenyl)-1-phenylethyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (C158). TLC system: 15% Methanol in dichloromethane Rf: 0.1 LCMS (ESI): m/z 651.2 [M+H]⁺

### Example 125: Synthesis of Compounds C165 and C159

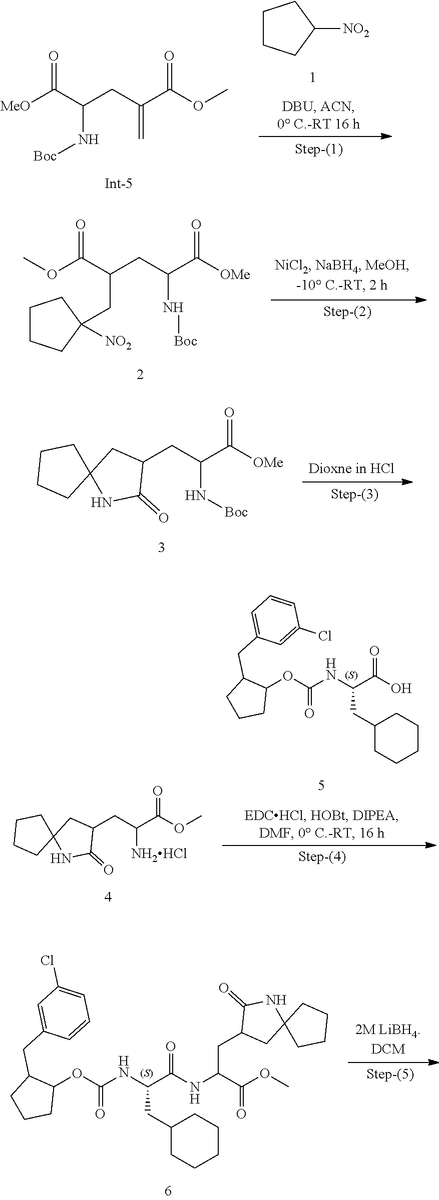

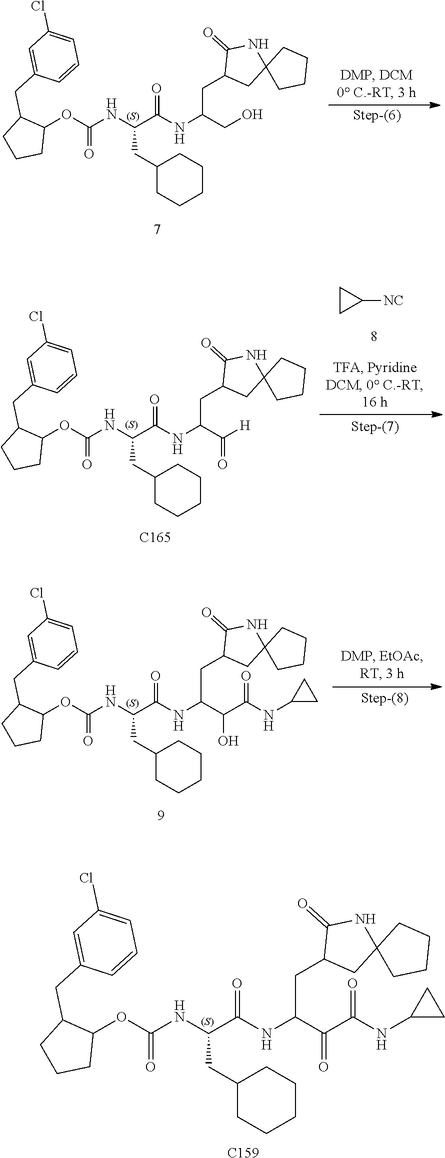

### Dimethyl 2-((tert-butoxycarbonyl)amino)-4-((1-nitrocyclopentyl)methyl)pentanedioate (2)

To a stirred solution of dimethyl 2-((tert-butoxycarbonyl)amino)-4-methylenepentanedioate (6.8 g, 14.34 mmol) in ACN, was added nitrocyclopentane (1) (1.5 g, 13.04 mmol) and DBU (3.6 mL, 13.44 mmol) at 0° C. and stirred at RT for 16 h. The progress of the reaction was monitored by TLC and LCMS. Reaction mixture was evaporated under reduced pressure. The crude residue was purified by normal phase chromatography to afford dimethyl 2-((tert-butoxycarbonyl)amino)-4-((1-nitrocyclopentyl)methyl)pentanedioate (2). TLC system: 10% Pet-ether/EtOAc R_{f}: 0.25 LCMS (ESI): m/z 403.2 (M+H)⁺

### Methyl 2-((tert-butoxycarbonyl)amino)-3-(2-oxo-1-azaspiro[4.4]nonan-3-yl)propanoate (3)

To a stirred solution of dimethyl dimethyl 2-((tert-butoxycarbonyl)amino)-4-((1-nitrocyclopentyl)methyl)pentanedioate (2) (4.3 g, 10.64 mmol) in methanol (50 mL) was added nickel chloride (1.3 g, 10.64 mmol), followed by sodiumborohydride (2 g, 53.21 mmol) at −10° C. and stirred at RT for 2 h. The progress of the reaction was monitored by TLC and LCMS. The reaction mixture was quenched with water (50 mL) and extracted with ethyl acetate (3×50 mL), combined organic layers were washed with water (2×50 ml), brine solution (50 mL), dried over sodium sulfate and evaporated under reduced pressure. The crude residue was purified by normal phase chromatography to afford methyl 2-((tert-butoxycarbonyl)amino)-3-(2-oxo-1-azaspiro[4.4]nonan-3-yl)propanoate (3). TLC system: 5% MeOH/DCM R_{f}: 0.2 LCMS (ESI): m/z 341.3 (M+H)⁺

### Methyl 2-amino-3-(2-oxo-1-azaspiro[4.4]nonan-3-yl)propanoate hydrochloride (4)

To a stirred solution of methyl 2-((tert-butoxycarbonyl)amino)-3-(2-oxo-1-azaspiro[4.4]nonan-3-yl)propanoate (3) (2.5 g, 3.75 mmol) in dioxane (25 mL) was added 4M HCl in dioxane (25 mL) at 0° C. and stirred at RT for 2 h. The progress of the reaction was monitored by TLC. Reaction mixture was evaporated under reduced pressure. The crude residue was trituration with n-pentane to afford methyl 2-amino-3-(2-oxo-1-azaspiro[4.4]nonan-3-yl)propanoate hydrochloride (4). TLC system: 10% MeOH/DCM R_{f}: 0.2

### Methyl 2-((2S)-2-((((2-(3-chlorobenzyl)cyclopentyl)oxy)carbonyl)amino)-3-cyclohexylpropanamido)-3-(2-oxo-1-azaspiro[4.4]nonan-3-yl)propanoate (6)

To a stirred solution of (2S)-2-((((2-(3-chlorobenzyl)cyclopentyl)oxy)carbonyl)amino)-3-cyclohexylpropanoic acid (5) (2 g, 4.91 mmol in DMF) at 0° C. was added EDC.HCl (1.4 g, 7.17 mmol), HOBT (900 mg, 6.66 mmol), DIPEA (2.7 mL, 3 Vol.) and methyl 2-amino-3-(2-oxo-1-azaspiro[4.4]nonan-3-yl)propanoate hydrochloride (4) (1.6 g, 5.89 mmol) simultaneously and stirred at room temperature for 16 h. The progress of the reaction was monitored by TLC and LCMS. After 16 h, reaction mixture was quenched with ice water (20 mL), extracted with ethyl acetate (2×30 mL), the combined organic layer was dried over sodium sulfate and evaporated under reduced pressure. The crude residue was purified by silica gel column by eluting with 50% ethyl acetate in pet ether to afford methyl 2-((2S)-2-((((2-(3-chlorobenzyl)cyclopentyl)oxy)carbonyl)amino)-3-cyclohexylpropanamido)-3-(2-oxo-1-azaspiro[4.4]nonan-3-yl)propanoate (6). TLC system: 5% Methanol in DCM R_{f}: 0.5 LCMS (ESI): m/z 630.8 (M+H)⁺

### 2-(3-Chlorobenzyl)cyclopentyl ((2S)-3-cyclohexyl-1-((1-hydroxy-3-(2-oxo-1-azaspiro[4.4]nonan-3-yl)propan-2-yl)amino)-1-oxopropan-2-yl)carbamate (7)

To a stirred solution of methyl 2-((2S)-2-((((2-(3-chlorobenzyl)cyclopentyl)oxy)carbonyl)amino)-3-cyclohexylpropanamido)-3-(2-oxo-1-azaspiro[4.4]nonan-3-yl)propanoate (6) (700 mg, 1.11 mmol) in DCM (10 mL) was added 2M LiBH₄ in THF (2.2 mL, 0.24 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford 2-(3-chlorobenzyl)cyclopentyl ((2S)-3-cyclohexyl-1-((1-hydroxy-3-(2-oxo-1-azaspiro[4.4]nonan-3-yl)propan-2-yl)amino)-1-oxopropan-2-yl)carbamate (7). TLC system: 5% MeOH in DCM R_{f} 0.3 LCMS (ESI): m/z 602.4 (M+H)⁺

### 2-(3-Chlorobenzyl)cyclopentyl ((2S)-3-cyclohexyl-1-oxo-1-((1-oxo-3-(2-oxo-1-azaspiro[4.4]nonan-3-yl)propan-2-yl)amino)propan-2-yl)carbamate (C165)

To a stirred solution of 3-chlorobenzyl 2-(3-chlorobenzyl)cyclopentyl ((2S)-3-cyclohexyl-1-((1-hydroxy-3-(2-oxo-1-azaspiro[4.4]nonan-3-yl)propan-2-yl)amino)-1-oxopropan-2-yl)carbamate (7) (200 mg, 0.33 mmol) in ethyl acetate (10 mL) was added Dess-Martin periodinane (422 mg, 0.99 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 2-(3-chlorobenzyl)cyclopentyl ((2S)-3-cyclohexyl-1-oxo-1-((1-oxo-3-(2-oxo-1-azaspiro[4.4]nonan-3-yl)propan-2-yl)amino)propan-2-yl)carbamate (C165). TLC system: 5% Methanol in DCM R_{f}: 0.4 LCMS (ESI): m/z 600.2 (M+H)⁺

### 2-(3-Chlorobenzyl)cyclopentyl ((2S)-3-cyclohexyl-1-((4-(cyclopropylamino)-3-hydroxy-4-oxo-1-(2-oxo-1-azaspiro[4.4]nonan-3-yl)butan-2-yl)amino)-1-oxopropan-2-yl)carbamate (9)

To a stirred solution 2-(3-chlorobenzyl)cyclopentyl ((2S)-3-cyclohexyl-1-oxo-1-((1-oxo-3-(2-oxo-1-azaspiro[4.4]nonan-3-yl)propan-2-yl)amino)propan-2-yl)carbamate (C165) (400 mg, 0.06 mmol) was dissolved in DCM (20 mL) added Pyridine (1.2 mL, 3 vol), isocyanocyclopropane (8) (66 mg, 1.29 mmol) sequentially at 0° C. and stirred for 10 min. To this was added TFA (0.05 mL, 0.66 mmol) at 0° C. and stirred at RT for 16 h. The progress of the reaction was monitored by TLC and LCMS. Reaction mixture was quenched with ice water (20 mL) and extracted with dichloromethane (2×15 mL). The organic layer was washed with 1N HCl (3×15 mL), brine solution (3×10 mL). The organic layer was dried over anhydrous Na₂SO₄ and evaporated under reduced pressure to afford 2-(3-chlorobenzyl)cyclopentyl ((2S)-3-cyclohexyl-1-((4-(cyclopropylamino)-3-hydroxy-4-oxo-1-(2-oxo-1-azaspiro[4.4]nonan-3-yl)butan-2-yl)amino)-1-oxopropan-2-yl)carbamate (9). TLC system: 5% Methanol/Dichloromethane Rf: 0.5 LCMS (ESI): m/z 685.2 [M+H]⁺

### 2-(3-Chlorobenzyl)cyclopentyl ((2S)-3-cyclohexyl-1-((4-(cyclopropylamino)-3,4-dioxo-1-(2-oxo-1-azaspiro[4.4]nonan-3-yl)butan-2-yl)amino)-1-oxopropan-2-yl)carbamate (C159)

To a stirred solution of 2-(3-chlorobenzyl)cyclopentyl ((2S)-3-cyclohexyl-1-((4-(cyclopropylamino)-3-hydroxy-4-oxo-1-(2-oxo-1-azaspiro[4.4]nonan-3-yl)butan-2-yl)amino)-1-oxopropan-2-yl)carbamate (9) (200 mg, 0.29 mmol) in EtOAc (10 mL) was added Dess-Martin periodinane (371 mg, 0.87 mmol) at 0° C. and stirred at RT for 3 h. The progress of the reaction was monitored by TLC and LCMS. Reaction mixture was filter through celite pad and washed with Ethyl acetate (25 mL) and filtrate was washed with hypo solution (3×10 mL) followed by saturated NaHCO₃ solution (3×10 mL). Organic layer was dried over anhydrous Na₂SO₄, filtered and concentrated to get crude residue. The crude compound was purified by prep HPLC to afford 2-(3-chlorobenzyl)cyclopentyl ((2S)-3-cyclohexyl-1-((4-(cyclopropylamino)-3,4-dioxo-1-(2-oxo-1-azaspiro[4.4]nonan-3-yl)butan-2-yl)amino)-1-oxopropan-2-yl)carbamate (C159). TLC system: 10% Methanol/Dichloromethane Rf: 0.4 LCMS (ESI): m/z 683.2 (M+H)⁺

### Example 126: Synthesis of Compounds C186 and C160

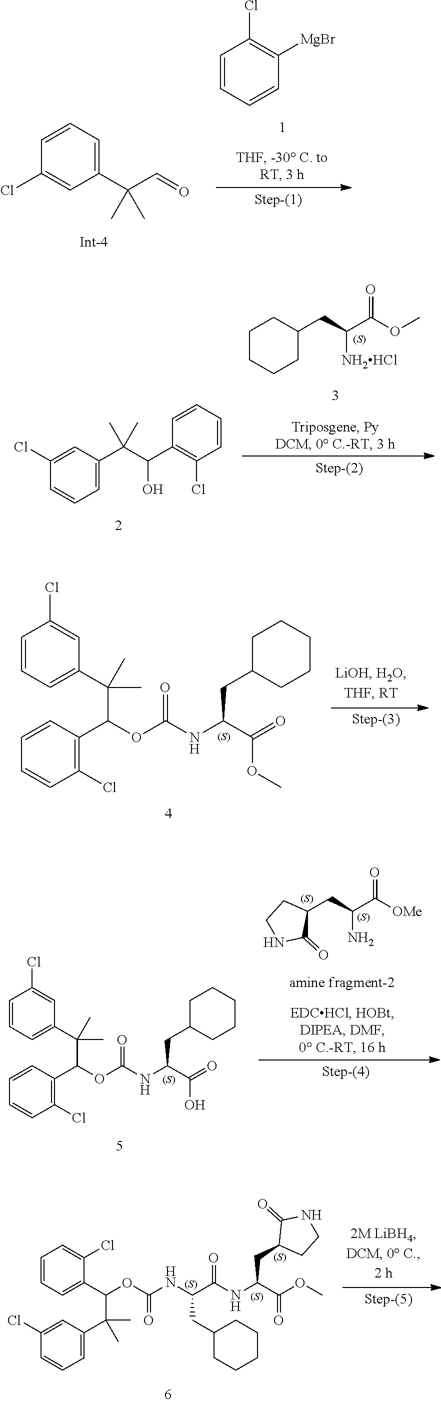

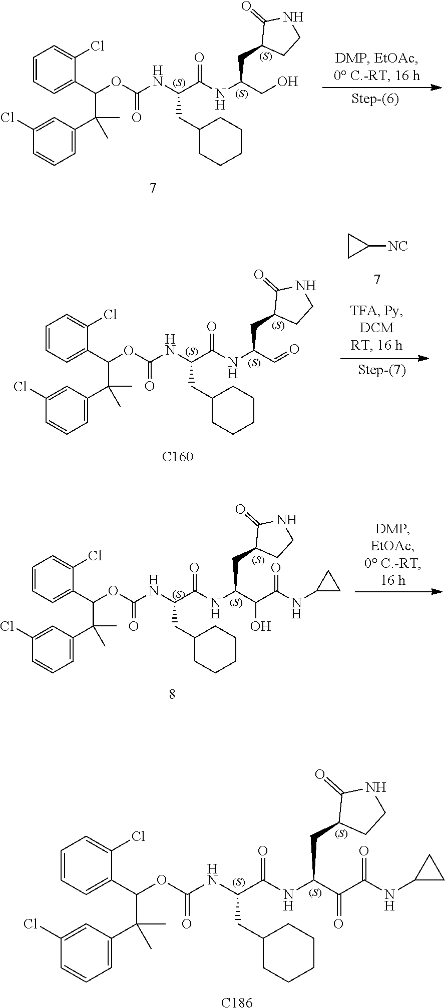

### 1-(2-Chlorophenyl)-2-(3-chlorophenyl)-2-methylpropan-1-ol (2)

To a stirred solution of (2-chlorophenyl) magnesium bromide in THF (1) (65 mL, 65.934 mmol) was added 2-(3-chlorophenyl)-2-methylpropanal (4 g, 21.978 mmol) in THF (50 mL) at −30° C. and stirred at RT for 3 h. The progress of the reaction was monitored by TLC. Reaction mixture was quenched with saturated ammonium chloride solution and extracted with ethyl acetate (2×50 mL). Combined organic layer and washed with water (2×50 mL), dried over sodium sulfate, concentrated under reduced pressure to afford crude residue, crude residue was purified by normal phase chromatography to afford 1-(2-chlorophenyl)-2-(3-chlorophenyl)-2-methylpropan-1-ol (2). TLC system: 5% Ethyl acetate in hexane Rf: 0.7

### Methyl (2S)-2-(((1-(2-chlorophenyl)-2-(3-chlorophenyl)-2-methylpropoxy) carbonyl) amino)-3-cyclohexylpropanoate (4)

To a stirred solution of 1-(2-chlorophenyl)-2-(3-chlorophenyl)-2-methylpropan-1-ol (2) (3.5 g, 11.904 mmol) and methyl (S)-2-amino-3-cyclohexylpropanoate (3) (2.64 g, 14.285 mmol) in DCM (40 mL) was added pyridine (3.8 mL, 1 vol) followed by triphosgene (1.76 g, 5.9523 mmol) with portion wise for 15 min at 0° C. and stirred at room temperature for 3 h. The progress of the reaction was monitored by TLC and LCMS. Reaction mixture was diluted with DCM and washed with 1N HCl (50 mL), brine (50 mL), organic layer was dried over sodium sulfate, filtered and evaporated under reduced pressure to afford crude; The crude residue was purified by combi-flash NP, compound eluted at 10% ethyl acetate in pet ether to afford methyl (2S)-2-(((1-(2-chlorophenyl)-2-(3-chlorophenyl)-2-methylpropoxy)carbonyl)amino)-3-cyclohexyl propanoate (4). TLC system: 5% Ethyl acetate in hexane Rf: 0.3 LCMS (ESI): m/z 528.36 [M+Na]⁺

### (2S)-2-(((1-(2-Chlorophenyl)-2-(3-chlorophenyl)-2-methylpropoxy) carbonyl) amino)-3-cyclohexylpropanoic acid (5)

To a stirred solution of methyl (2S)-2-(((1-(2-chlorophenyl)-2-(3-chlorophenyl)-2-methylpropoxy)carbonyl)amino)-3-cyclohexylpropanoate (4) (3.8 g, 7.524 mmol) in THF (30 mL), water (10 mL) was added lithium hydroxide (541 mg, 22.57 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-(((1-(2-chlorophenyl)-2-(3-chlorophenyl)-2-methylpropoxy)carbonyl)amino)-3-cyclohexylpropanoic acid (5). TLC system: 80% Ethyl acetate in hexane Rf: 0.2 LCMS (ESI): m/z 514.36 [M+Na]⁺

### Methyl (2S)-2-((2S)-2-(((1-(2-chlorophenyl)-2-(3-chlorophenyl)-2-methylpropoxy) carbonyl) amino)-3-cyclohexylpropanamido)-3-((S)-2-oxopyrrolidin-3-yl) propanoate (6)

To a stirred solution of (2S)-2-(((1-(2-chlorophenyl)-2-(3-chlorophenyl)-2-methylpropoxy)carbonyl)amino)-3-cyclohexylpropanoic acid (5) (2 g, 4.073 mmol) DMF (15 mL) added EDC.HCl (1.16 g, 6.10 mmol), HOBt (824 mg, 6.10 mmol), DIPEA (2.2 mL, 12.21 mmol) and methyl (S)-2-amino-3-((S)-2-oxopyrrolidin-3-yl)propanoate hydrochloride (amine fragment-2) (910 mg, 4.88 mmol) at 0° C. simultaneously and stirred at room temperature for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash NP, compound eluted at 60% ethyl acetate in pet ether to afford methyl (2S)-2-((2S)-2-(((1-(2-chlorophenyl)-2-(3-chloro phenyl)-2-methylpropoxy)carbonyl)amino)-3-cyclohexylpropanamido)-3-((S)-2-oxopyrrolidin-3-yl)propanoate (6). TLC system: 80% Ethyl acetate in Pet ether Rf: 0.5 LCMS (ESI): m/z 660.43 [M+H]⁺

### 1-(2-Chlorophenyl)-2-(3-chlorophenyl)-2-methylpropyl ((S)-3-cyclohexyl-1-(((S)-1-hydroxy-3-((S)-2-oxopyrrolidin-3-yl) propan-2-yl) amino)-1-oxopropan-2-yl) carbamate (7)

To a stirred solution of methyl (2S)-2-((2S)-2-(((1-(2-chlorophenyl)-2-(3-chlorophenyl)-2-methylpropoxy)carbonyl)amino)-3-cyclohexylpropanamido)-3-((S)-2-oxopyrrolidin-3-yl)propanoate (6) (1.5 g, 2.276 mmol) in DCM (20 mL) was added 2M LiBH₄ in THF (2.2 mL, 4.552 mmol) at 0° C. and the reaction mixture stirred for 2 h at 0° C. The progress of the reaction was monitored by TLC and LCMS. Reaction mixture was quenched with saturated ammonium chloride solution (40 mL) and extracted with DCM (2×20 mL). Organic layer was washed with brine solution (30 mL), dried over Na₂SO₄ and concentrated to afford 1-(2-chlorophenyl)-2-(3-chlorophenyl)-2-methylpropyl ((S)-3-cyclohexyl-1-(((S)-1-hydroxy-3-((S)-2-oxopyrrolidin-3-yl) propan-2-yl) amino)-1-oxopropan-2-yl)carbamate (7). TLC system: 10% Methanol in dichloromethane Rf: 0.3 LCMS (ESI): m/z 632.38 [M+H]⁺

### 11-(2-Chlorophenyl)-2-(3-chlorophenyl)-2-methylpropyl ((S)-3-cyclohexyl-1-oxo-1-(((S)-1-oxo-3-((S)-2-oxopyrrolidin-3-yl) propan-2-yl) amino) propan-2-yl) carbamate (C160)

To a stirred solution of 1-(2-chlorophenyl)-2-(3-chlorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (7) (100 mg, 0.15 mmol) was dissolved in ethyl acetate (10 mL) was added Dess-Martin periodinane (201 mg, 0.47 mmol) at 0° C. and stirred at RT for 16 h. Reaction mixture was filtered through celite bed and washed with ethyl acetate (20 mL). Filtrate was washed with sat. Hypo solution (3×30 mL) followed by sat. NaHCO₃ solution (3×30 mL). Organic layer was dried over anhydrous Na₂SO₄, filtered and concentrated to get crude compound. The crude compound was purified by combi flash NP; compound eluted at 5% methanol dichloromethane to afford 1-(2-chlorophenyl)-2-(3-chlorophenyl)-2-methylpropyl ((S)-3-cyclohexyl-1-oxo-1-(((S)-1-oxo-3-((S)-2-oxo pyrrolidin-3-yl)propan-2-yl) amino) propan-2-yl) carbamate (C160). TLC system: 10% Methanol in dichloromethane Rf: 0.5 LCMS (ESI): m/z 630.2 (M+H)⁺

### 1-(2-Chlorophenyl)-2-(3-chlorophenyl)-2-methylpropyl ((2S)-3-cyclohexyl-1-(((2S)-4-(cyclopropylamino)-3-hydroxy-4-oxo-1-((S)-2-oxopyrrolidin-3-yl) butan-2-yl) amino)-1-oxopropan-2-yl) carbamate (9)

To a stirred solution of 1-(2-chlorophenyl)-2-(3-chlorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl) carbamate (C160) (250 mg, 0.39 mmol) in DCM (10 mL) was added isocyanocyclopropane (8) (39 mg, 0.59 mmol), pyridine (0.5 mL, 2 vol) followed by TFA (0.1 mL, 0.79 mmol) at 0° C. and stirred at RT for 16 h. The progress of the reaction was monitored by TLC and LCMS. After 16 h, the reaction mixture was diluted with dichloromethane and washed with 1N HCl solution (30 mL) followed by brine (20 mL), dried over sodium sulfate, concentrated under reduce pressure to afford crude residue. This residue was triturated with di ethyl ether (30 mL) to afford 1-(2-chlorophenyl)-2-(3-chlorophenyl)-2-methylpropyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (9). TLC system: 10% Methanol in dichloromethane Rf: 0.4 LCMS (ESI): m/z 715.57 [M+H]⁺

### 1-(2-Chlorophenyl)-2-(3-chlorophenyl)-2-methylpropyl ((S)-3-cyclohexyl-1-(((S)-4-(cyclopropylamino)-3,4-dioxo-1-((S)-2-oxopyrrolidin-3-yl) butan-2-yl) amino)-1-oxopropan-2-yl) carbamate (C186)

To a stirred solution of 1-(2-chlorophenyl)-2-(3-chlorophenyl)-2-methylpropyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (9) (170 mg, 0.23 mmol) was dissolved in ethyl acetate (10 mL) was added Dess-Martin periodinane (201 mg, 0.47 mmol) at 0° C. and stirred at RT for 16 h. Reaction mixture was filtered through celite bed and washed with ethyl acetate (20 mL). Filtrate was washed with sat. Hypo solution (3×20 mL) followed by sat. NaHCO₃ solution (3×20 mL). Organic layer was dried over anhydrous Na₂SO₄, filtered and concentrated to get crude compound. The crude residue was purified by combi-flash NP, compound eluted at 6% methanol in dichloromethane to afford 1-(2-chlorophenyl)-2-(3-chlorophenyl)-2-methylpropyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (C186). TLC system: 10% Methanol in dichloromethane Rf: 0.5 LCMS (ESI): m/z 713.2 [M+H]⁺

### Example 127: Synthesis of Compound C161

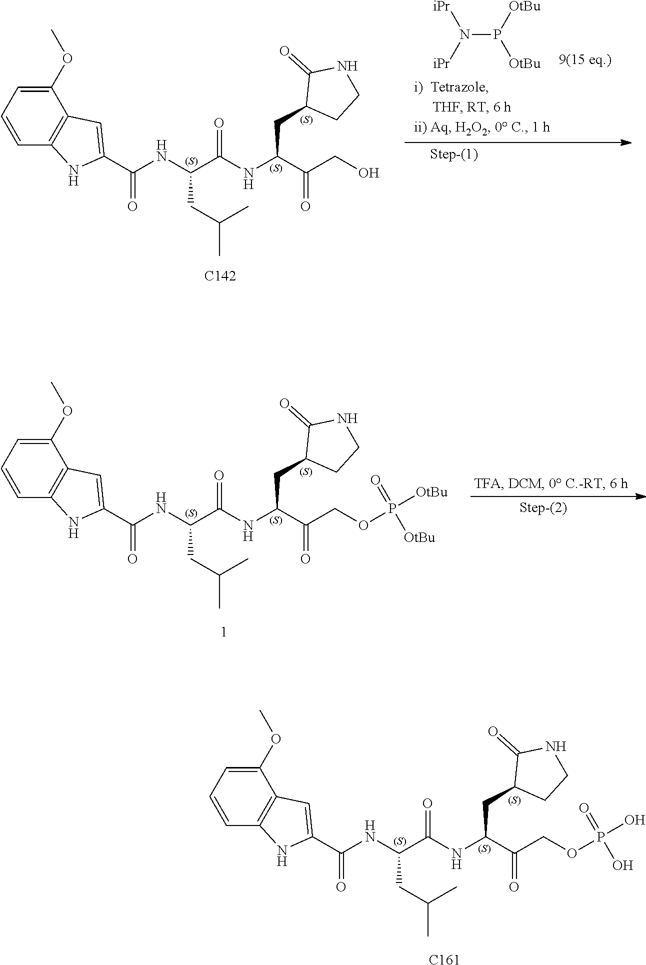

### Di-tert-butyl ((S)-3-((S)-2-(4-methoxy-1H-indole-2-carboxamido)-4-methylpentanamido)-2-oxo-4-((S)-2-oxopyrrolidin-3-yl)butyl) phosphate (1)

To a solution of N—((S)-1-(((S)-4-hydroxy-3-oxo-1-((S)-2-oxopyrrolidin-3-yl)butan-2-yl)amino)-4-methyl-1-oxopentan-2-yl)-4-methoxy-1H-indole-2-carboxamide (C142) (200 mg, 0.423 mmol) in THF (4 mL) was added 1H-tetrazole (300 mg, 4.23 mmol) and di-tert-butyl diethylphosphoramidite (2 mL, 6.35 mmol) at room temperature. The reaction was stirred at RT for 4 hours then 30% Aq. hydrogen peroxide (0.5 mL) was added at 0° C. The mixture was stirred at 25° C. for 1 hour. Progression of the reaction was monitored by TLC and LCMS. After that, the reaction mixture was quenched with 10% sodium meta bisulphite solution (Na₂S₂O₅) (10 mL). Then the layers were separated and washed with sat. NaHCO₃ solution (2×20 mL). The organic layer was dried over Na₂SO₄ and evaporated under vacuum. This crude material was purified by normal phase column chromatography to afford di-tert-butyl ((S)-3-((S)-2-(4-methoxy-1H-indole-2-carboxamido)-4-methylpentanamido)-2-oxo-4-((S)-2-oxopyrrolidin-3-yl)butyl) phosphate (1). TLC system: 10% MeOH in DCM R_{f}: 0.55 LCMS (ESI): m/z 665.87 [M+H]⁺

### (S)-3-((S)-2-(4-methoxy-1H-indole-2-carboxamido)-4-methylpentanamido)-2-oxo-4-((S)-2-oxopyrrolidin-3-yl)butyl dihydrogen phosphate (C161)

A solution of di-tert-butyl ((S)-3-((S)-2-(4-methoxy-1H-indole-2-carboxamido)-4-methylpentanamido)-2-oxo-4-((S)-2-oxopyrrolidin-3-yl)butyl) phosphate (1) (100 mg, 0.15 mmol) in DCM (2 mL) was placed under an atmosphere of N2 and cooled to 0° C. Then trifluoroacetic acid (0.2 ml) was added and the reaction stirred at room temperature under nitrogen for 6 hours. Progression of the reaction was monitored by TLC and LCMS. The mixture was concentrated in vacuo and then concentrated from dichloromethane (2×5 mL) and diethyl ether (2×5 mL) to remove residual acid. The crude compound was purified by reverse phase prep-HPLC by using 0.1% TFA in water and acetonitrile as gradients to afford (S)-3-((S)-2-(4-methoxy-1H-indole-2-carboxamido)-4-methylpentanamido)-2-oxo-4-((S)-2-oxopyrrolidin-3-yl)butyl dihydrogen phosphate (C161). TLC system: 10% MeOH in DCM R_{f}: 0.4 LCMS (ESI): m/z 553.1 [M+H]⁺

### Example 128: Synthesis of Compounds C162 and C172

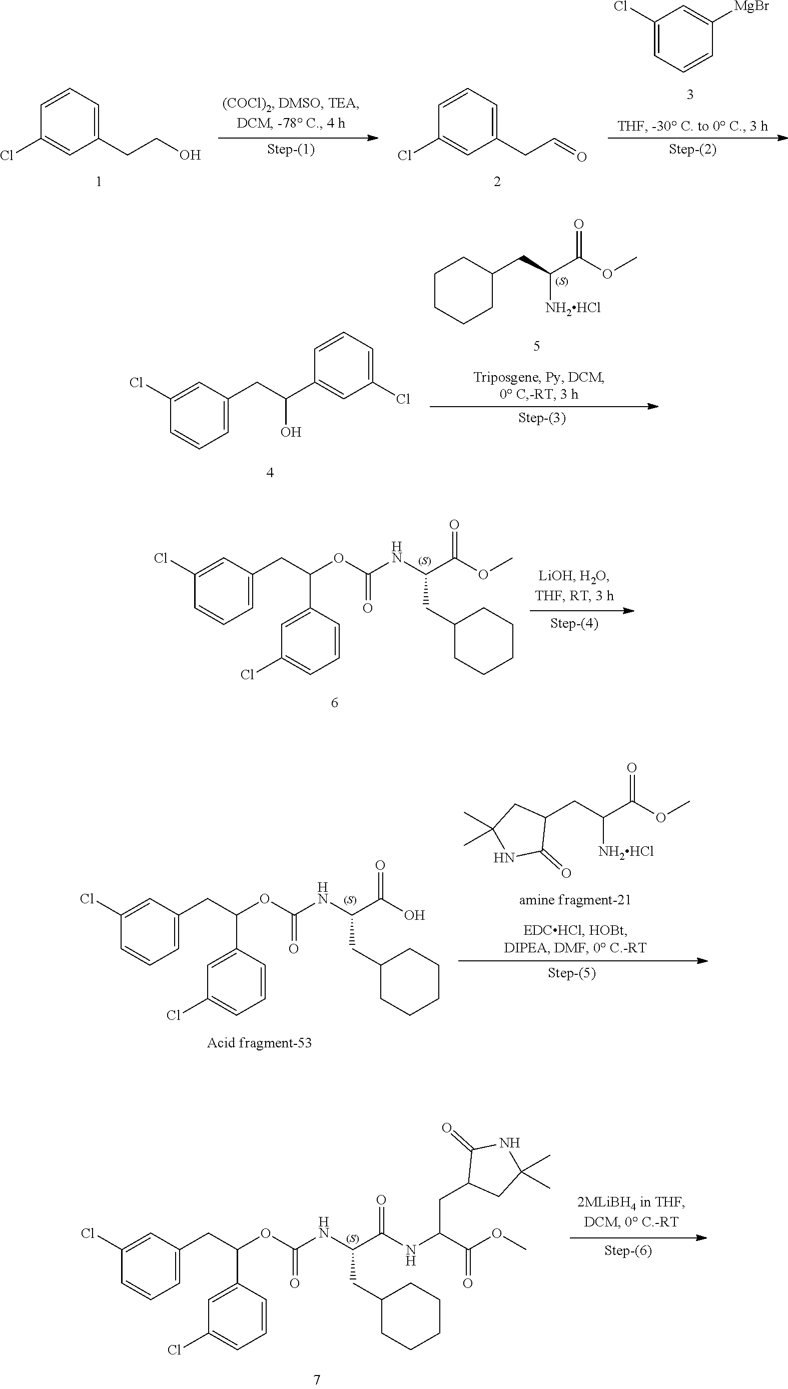

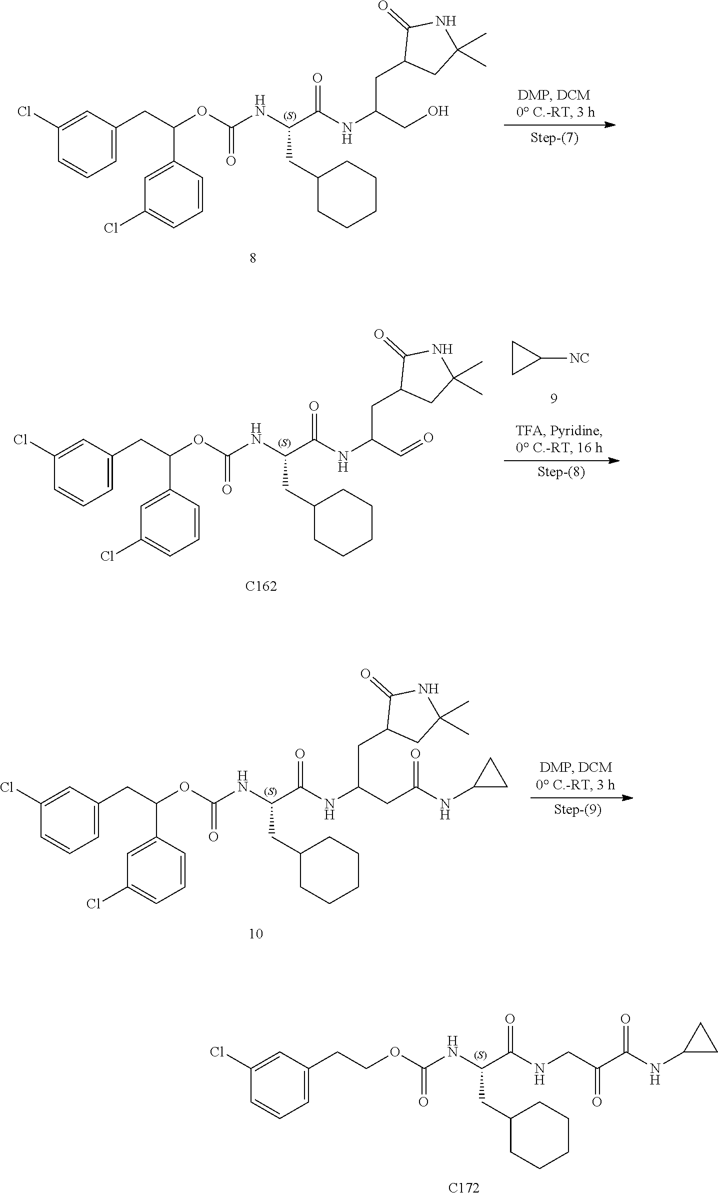

### 2-(3-chlorophenyl) acetaldehyde (2)

To a stirred solution of DMSO (2.7 mL, 38.46 mmol) in DCM (30 mL) was added oxalyl chloride (2.5 mL, 28.84 mmol) at −78° C. and stirred for 30 min then added 2-(3-chlorophenyl) ethan-1-ol (1) (3 g, 19.23 mmol) in DCM at −78° C. and continued for 2 h after that TEA (8.3 mL, 57.69 mmol) was added at same temperature and stirred at RT for 3 h. The progress of the reaction was monitored by TLC. Reaction mixture was diluted with DCM (100 mL) and washed with ice cold water (3×60 mL), dried over sodium sulfate, concentrated under reduced pressure to afford crude 2-(3-chlorophenyl)acetaldehyde (2). This crude was used for next step. TLC system: 10% Ethyl acetate in hexane Rf: 0.4

### 1,2-Bis(3-Chlorophenyl)ethan-1-ol

To a stirred solution of (3-chlorophenyl)magnesium bromide (3) (58 mL, 58.44 mmol) THF (50 mL) wad added 2-(3-chlorophenyl)acetaldehyde (2) (3 g, 19.48 mmol) at −30° C. and stirred at RT for 3 h. Reaction progress was monitored by TLC and LCMS. Reaction mixture was quenched with sat. ammonium chloride and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% ethyl acetate in hexane to afford 1,2-bis(3-chlorophenyl)ethan-1-ol (4). TLC system: 10% Ethyl acetate in hexane Rf: 0.3 LCMS (ESI): m/z 249.11 [M−OH]⁺

### Methyl (2S)-2-(((1,2-bis (3-chlorophenyl) ethoxy) carbonyl) amino)-3-cyclohexylpropanoate (6)

To a stirred solution of 1,2-bis(3-chlorophenyl)ethan-1-ol (4) (2.3 g, 8.64 mmol) and methyl (S)-2-amino-3-cyclohexylpropanoate hydrochloride (5) (1.9 g, 10.37 mmol) in DCM (40 mL) was added pyridine (2.3 mL, 1 vol) followed by triphosgene (1.27 g, 4.32 mmol) at 0° C. and stirred at room temperature for 16 h. The progress of the reaction was monitored by TLC and LCMS. Reaction mixture was diluted with DCM and washed with 1N HCl (50 mL), organic layer was dried over sodium sulfate, filtered and evaporated under reduced pressure. The crude residue was purified by combi-flash, compound eluted at 7% ethyl acetate in pet ether to afford methyl (2S)-2-(((1,2-bis (3-chlorophenyl) ethoxy) carbonyl) amino)-3-cyclohexylpropanoate (6). TLC system: 10% Ethyl acetate in hexane Rf: 0.4 LCMS (ESI): m/z 500.28 [M+Na]⁺

### (2S)-2-(((1,2-bis (3-chlorophenyl) ethoxy) carbonyl) amino)-3-cyclohexylpropanoic acid (acid-fragment-53)

To a stirred solution of methyl (2S)-2-(((1,2-bis (3-chlorophenyl) ethoxy) carbonyl) amino)-3-cyclohexylpropanoate (6) (2.1 g, 4.40 mmol) in THF (10 mL), water (5 mL) was added lithium hydroxide (316 mg, 13.20 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×30 mL), dried over sodium sulfate, concentrated under reduced pressure to afford (2S)-2-(((1,2-bis (3-chlorophenyl) ethoxy) carbonyl) amino)-3-cyclohexylpropanoic acid (acid fragment-53). TLC system: 100% EtOAc Rf: 0.1 LCMS (ESI): m/z 486.25 [M+Na]⁺

### Methyl 2-((2S)-2-(((1,2-bis (3-chlorophenyl) ethoxy) carbonyl) amino)-3-cyclohexylpropanamido)-3-(5,5-dimethyl-2-oxopyrrolidin-3-yl) propanoate (7)

To a stirred solution of (2S)-2-(((1,2-bis(3-chlorophenyl)ethoxy)carbonyl)amino)-3-cyclohexylpropanoic acid (acid fragment-53) (1 g, 2.07 mmol) DMF (10 mL) added EDC.HCl (595 mg, 3.11 mmol), HOBt (420 mg, 3.11 mmol), DIPEA (0.8 mL, 6.23 mmol) and methyl 2-amino-3-(5,5-dimethyl-2-oxopyrrolidin-3-yl)propanoate hydrochloride (amine fragment-21) (623 mg, 2.49 mmol) at 0° C. simultaneously and stirred at room temperature for 16 h. Reaction mixture was quenched with ice water (80 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% methanol in dichloromethane to afford methyl 2-((2S)-2-(((1,2-bis(3-chlorophenyl)ethoxy)carbonyl)amino)-3-cyclohexylpropanamido)-3-(5,5-dimethyl-2-oxopyrrolidin-3-yl)propanoate (7). TLC system: 10% Methanol in dichloromethane Rf: 0.4 LCMS (ESI): m/z 660.2 [M+H]⁺

### 1,2-Bis(3-Chlorophenyl)ethyl ((2S)-3-cyclohexyl-1-((1-(5,5-dimethyl-2-oxopyrrolidin-3-yl)-3-hydroxypropan-2-yl)amino)-1-oxopropan-2-yl)carbamate (8)

To a stirred solution of methyl 2-((2S)-2-(((1,2-bis(3-chlorophenyl)ethoxy) carbonyl) amino)-3-cyclohexylpropanamido)-3-(5,5-dimethyl-2-oxopyrrolidin-3-yl)propanoate (7) (950 mg, 1.44 mmol) in DCM (10 mL) was added 2M LiBH₄ in THF (1.4 mL, 2.88 mmol) at 0° C. and the reaction mixture stirred for 2 h at 0° C. The progress of the reaction was monitored by TLC and LCMS. Reaction mixture was quenched with sat. ammonium chloride solution (30 mL) and extracted with DCM (2×20 mL). Organic layer was washed with brine solution (30 mL), dried over Na₂SO₄ and concentrated to afford 1,2-bis(3-chlorophenyl) ethyl ((2S)-3-cyclohexyl-1-((1-(5,5-dimethyl-2-oxopyrrolidin-3-yl)-3-hydroxypropan-2-yl)amino)-1-oxopropan-2-yl) carbamate (8). TLC system: 10% Methanol in dichloromethane Rf: 0.2 LCMS (ESI): m/z 632.50 [M+H]⁺

### 1,2-Bis(3-Chlorophenyl)ethyl ((2S)-3-cyclohexyl-1-((1-(5,5-dimethyl-2-oxopyrrolidin-3-yl)-3-oxopropan-2-yl)amino)-1-oxopropan-2-yl)carbamate (C162)

To a stirred solution of 1,2-bis(3-chlorophenyl)ethyl ((2S)-3-cyclohexyl-1-((1-(5,5-dimethyl-2-oxo pyrrolidin-3-yl)-3-hydroxypropan-2-yl)amino)-1-oxo propan-2-yl)carbamate (8) (400 mg, 0.63 mmol) was dissolved in dichloromethane (10 mL) was added Dess-Martin periodinane (537 mg, 1.26 mmol) at 0° C. and stirred at RT for 3 h. Reaction mixture was diluted with DCM (20 mL) and washed with sat. Hypo solution (3×30 mL) followed by sat. NaHCO₃ solution (3×30 mL). Organic layer was dried over anhydrous Na₂SO₄, filtered and concentrated to get crude compound. The crude compound was purified by combi-flash NP, compound eluted at 5% methanol in dichloromethane to afford 1,2-bis(3-chlorophenyl)ethyl ((2S)-3-cyclohexyl-1-((1-(5,5-dimethyl-2-oxopyrrolidin-3-yl)-3-oxopropan-2-yl)amino)-1-oxopropan-2-yl)carbamate (C162). TLC system: 10% Methanol in dichloromethane Rf: 0.5 LCMS (ESI): m/z 630.1 (M+H)⁺

### 1,2-Bis(3-chlorophenyl)ethyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (10)

To a stirred solution of 1,2-bis(3-chlorophenyl)ethyl ((2S)-3-cyclohexyl-1-((1-(5,5-dimethyl-2-oxopyrrolidin-3-yl)-3-oxopropan-2-yl)amino)-1-oxopropan-2-yl)carbamate (C162) (350 mg, 0.55 mmol) in DCM (5 mL), was added Pyridine (1 mL) was added isocyanocyclopropane (9) (55 mg, 0.83 mmol), was added TFA (0.3 mL) at 0° C. and stirred at RT for 16 h. The progress of the reaction was monitored by TLC and LCMS. Reaction mixture was quenched with ice water (50 mL) and extracted with ethyl acetate (2×30 mL). Combined the organic layer, dried over sodium sulfate, concentrated under reduce pressure to afford crude residue. This residue was triturated with di ethyl ether (30 mL) for 3 times to afford 1,2-bis(3-chlorophenyl)ethyl ((2S)-3-cyclohexyl-1-((4-(cyclopropylamino)-1-(5,5-dimethyl-2-oxo pyrolidin-3-yl)-3-hydroxy-4-oxobutan-2-yl) amino)-1-oxopropan-2-yl) carbamate (10). TLC system: 15% Methanol in dichloromethane Rf: 0.1 LCMS (ESI): m/z 669.59 [M+H]⁺

### 1,2-Bis (3-Chlorophenyl) ethyl ((2S)-3-cyclohexyl-1-((4-(cyclopropylamino)-1-(5,5-dimethyl-2-oxopyrrolidin-3-yl)-3,4-dioxobutan-2-yl) amino)-1-oxopropan-2-yl)carbamate (C172)

To a stirred solution of 1,2-bis(3-chlorophenyl)ethyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (10) (280 mg, 0.39 mmol) was dissolved in dichloromethane (10 mL) was added Dess-Martin periodinane (332 mg, 0.78 mmol) at 0° C. and stirred at RT for 3 h. Reaction mixture was diluted with DCM (30 mL) and washed with sat. Hypo solution (3×30 mL) followed by sat. NaHCO₃ solution (3×30 mL). Organic layer was dried over anhydrous Na₂SO₄, filtered and concentrated to get crude compound. The crude compound was purified by prep HPLC to afford 1,2-bis(3-chlorophenyl)ethyl ((2S)-3-cyclohexyl-1-((4-(cyclopropylamino)-1-(5,5-dimethyl-2-oxopyrrolidin-3-yl)-3,4-dioxobutan-2-yl)amino)-1-oxopropan-2-yl)carbamate (C172). TLC system: 10% Methanol in dichloromethane Rf: 0.5 LCMS (ESI): m/z 713.2 (M+H)⁺

### Example 129: Synthesis of Compound C163

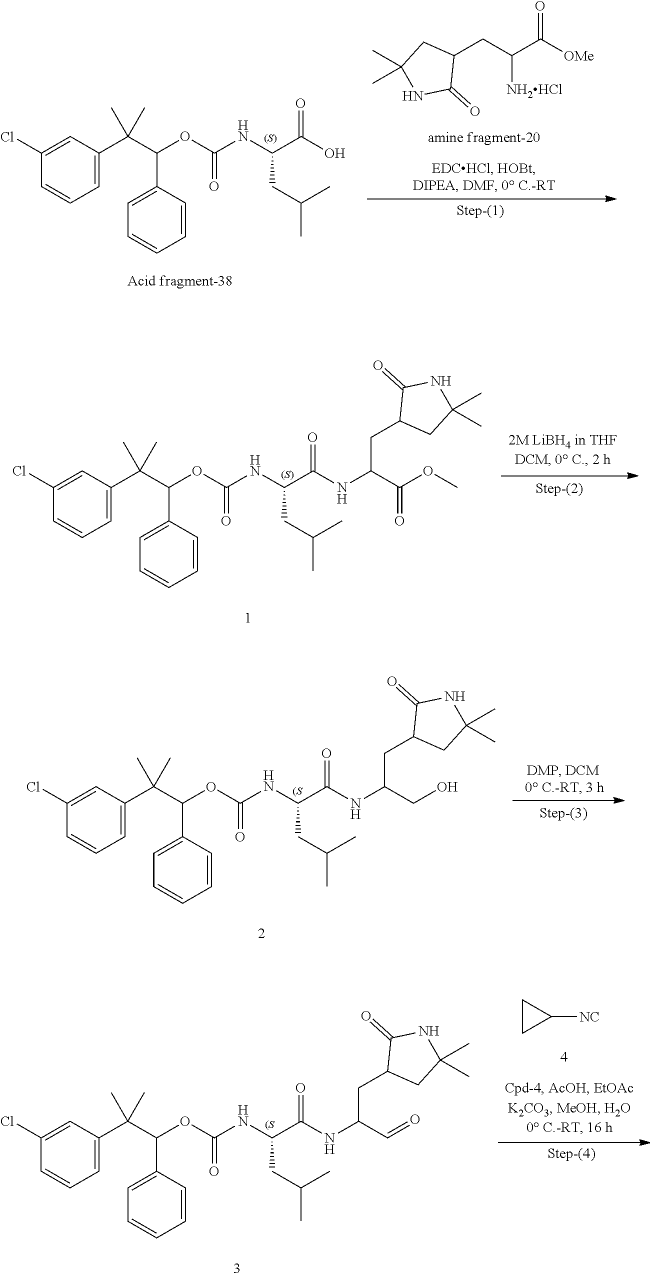

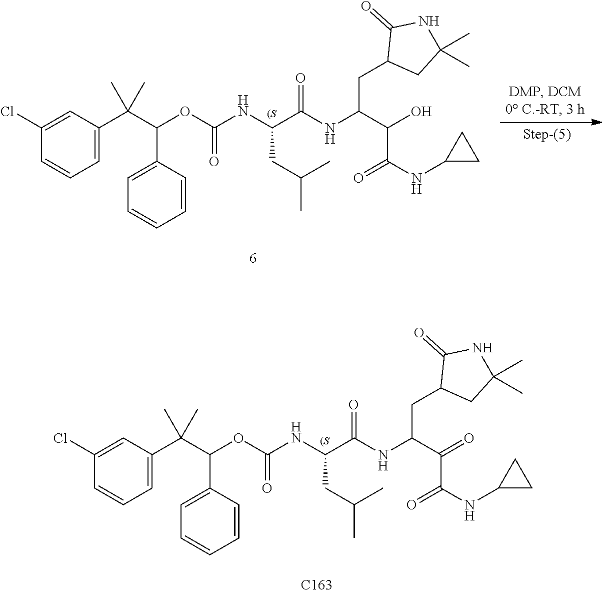 [SMILES: CC(C)C[C@H](NC(=O)OC(c1ccccc1)C(C)(C)c1cccc(Cl)c1)C(=O)NC(CC1CC(C)(C)NC1=O)C(O)C(=O)NC1CC1 ; "6" ; CC(C)C[C@H](NC(=O)OC(c1ccccc1)C(C)(C)c1cccc(Cl)c1)C(=O)NC(CC1CC(C)(C)NC1=O)C(=O)C(=O)NC1CC1 ; "C163"]

### Methyl 2-((2S)-2-(((2-(3-chlorophenyl)-2-methyl-1-phenylpropoxy) carbonyl) amino)-4-methylpentanamido)-3-(5,5-dimethyl-2-oxopyrrolidin-3-yl) propanoate (1)

To a stirred solution of ((2-(3-chlorophenyl)-2-methyl-1-phenylpropoxy)carbonyl)-L-leucine (Acid fragment-38) (1.6 g, 3.83 mmol) DMF (16 mL) added EDC.HCl (1 g, 5.75 mmol), HOBt (0.77 g, 5.75 mmol), DIPEA (1.6 mL, 11.50 mmol) and methyl 2-amino-3-(5,5-dimethyl-2-oxopyrrolidin-3-yl)propanoate hydrochloride (amine fragment-20) (1.15 g, 4.60 mmol) at 0° C. simultaneously and stirred at room temperature for 16 h. Reaction mixture was quenched with ice water (80 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% methanol in dichloromethane to afford methyl 24(2S)-2-(((2-(3-chlorophenyl)-2-methyl-1-phenylpropoxy) carbonyl) amino)-4-methylpentanamido)-3-(5,5-dimethyl-2-oxopyrrolidin-3-yl) propanoate (1). TLC system: 10% Methanol in dichloromethane Rf: 0.4 LCMS (ESI): m/z 614.48 [M+H]⁺

### 2-(3-Chlorophenyl)-2-methyl-1-phenylpropyl ((2S)-1-((1-(5,5-dimethyl-2-oxopyrrolidin-3-yl)-3-hydroxypropan-2-yl) amino)-4-methyl-1-oxopentan-2-yl) carbamate (2)

To a stirred solution of methyl 2-((2S)-2-(((2-(3-chlorophenyl)-2-methyl-1-phenyl propoxy)carbonyl)amino)-4-methylpentanamido)-3-(5,5-dimethyl-2-oxopyrrolidin-3-yl) propanoate (1) (350 mg, 0.57 mmol) in DCM (5 mL) was added 2M LiBH₄ in THF (0.6 mL, 1.14 mmol) at 0° C. and the reaction mixture stirred for 2 h at 0° C. The progress of the reaction was monitored by TLC and LCMS. Reaction mixture was quenched with sat. ammonium chloride solution (20 mL) and extracted with DCM (2×20 mL). Organic layer was washed with brine solution (30 mL), dried over Na₂SO₄ and concentrated to afford 2-(3-chlorophenyl)-2-methyl-1-phenylpropyl ((2S)-1-((1-(5,5-dimethyl-2-oxopyrrolidin-3-yl)-3-hydroxypropan-2-yl) amino)-4-methyl-1-oxopentan-2-yl)carbamate (2). TLC system: 10% Methanol in dichloromethane Rf: 0.2 LCMS (ESI): m/z 586.56 [M+H]⁺

### 2-(3-Chlorophenyl)-2-methyl-1-phenylpropyl ((2S)-1-((1-(5,5-dimethyl-2-oxopyrrolidin-3-yl)-3-oxopropan-2-yl) amino)-4-methyl-1-oxopentan-2-yl) carbamate (3)

To a stirred solution of 2-(3-chlorophenyl)-2-methyl-1-phenylpropyl ((2S)-1-((1-(5,5-dimethyl-2-oxopyrrolidin-3-yl)-3-hydroxypropan-2-yl)amino)-4-methyl-1-oxopentan-2-yl) carbamate (2) (300 mg, 0.51 mmol) was dissolved in dichloromethane (5 mL) was added Dess-Martin periodinane (519 mg, 1.02 mmol) at 0° C. and stirred at RT for 3 h. Reaction mixture was diluted with DCM (20 mL) and washed with sat. Hypo solution (3×20 mL) followed by sat. NaHCO₃ solution (3×20 mL). Organic layer was dried over anhydrous Na₂SO₄, filtered and concentrated to get crude compound. The crude compound was purified by prep HPLC to afford 2-(3-chlorophenyl)-2-methyl-1-phenylpropyl ((2S)-1-((1-(5,5-dimethyl-2-oxopyrrolidin-3-yl)-3-hydroxypropan-2-yl) amino)-4-methyl-1-oxopentan-2-yl) carbamate (3). TLC system: 10% Methanol in dichloromethane Rf: 0.5 LCMS (ESI): m/z 584.55 (M+H)⁺

### 2-(3-Chlorophenyl)-2-methyl-1-phenylpropyl ((2S)-1-((4-(cyclopropylamino)-1-(5,5-dimethyl-2-oxopyrrolidin-3-yl)-3-hydroxy-4-oxobutan-2-yl) amino)-4-methyl-1-oxopentan-2-yl) carbamate (5)

To a stirred solution of 2-(3-chlorophenyl)-2-methyl-1-phenylpropyl ((2S)-1-((1-(5,5-dimethyl-2-oxopyrrolidin-3-yl)-3-oxopropan-2-yl)amino)-4-methyl-1-oxopentan-2-yl)carbamate (3) (250 mg, 0.42 mmol) in ethyl acetate (10 mL) was added acetic acid (0.5 mL) was added isocyanocyclopropane (4) (56 mg, 0.83 mmol) at 0° C. and stirred at RT for 16 h. The progress of the reaction was monitored by TLC and LCMS. After 16 h, the reaction mixture was concentrated to get the crude; crude residue was dissolved in methanol (10 ml), water (5 mL) was added potassium carbonate (80 mg, 0.58 mmol) at RT and stirred at room temperature for 16 h. The progress of the reaction was monitored by TLC and LCMS. Reaction mixture completely distilled under reduced pressure, crude compound washed with aq. 1N HCl solution and extracted with ethyl acetate (2×30 mL), dried over sodium sulfate, concentrated under reduce pressure to afford crude residue. This residue was triturated with di ethyl ether (30 mL) for 3 times to afford 2-(3-chlorophenyl)-2-methyl-1-phenylpropyl ((2S)-1-((4-(cyclopropylamino)-1-(5,5-dimethyl-2-oxopyrrolidin-3-yl)-3-hydroxy-4-oxobutan-2-yl)amino)-4-methyl-1-oxopentan-2-yl)carbamate (5). TLC system: 10% Methanol in dichloromethane Rf: 0.1 LCMS (ESI): m/z 669.59 [M+H]⁺

### 2-(3-Chlorophenyl)-2-methyl-1-phenylpropyl ((2S)-1-((4-(cyclopropylamino)-1-(5,5-dimethyl-2-oxopyrrolidin-3-yl)-3,4-dioxobutan-2-yl) amino)-4-methyl-1-oxopentan-2-yl) carbamate (C163)

To a stirred solution of 2-(3-chlorophenyl)-2-methyl-1-phenylpropyl ((2S)-1-((4-(cyclopropylamino)-1-(5,5-dimethyl-2-oxopyrrolidin-3-yl)-3-hydroxy-4-oxobutan-2-yl)amino)-4-methyl-1-oxopentan-2-yl)carbamate (5) (200 mg, 0.29 mmol) was dissolved in dichloromethane (5 mL) was added Dess-Martin periodinane (310 mg, 0.59 mmol) at 0° C. and stirred at RT for 3 h. Reaction mixture was diluted with DCM (20 mL) and washed with sat. Hypo solution (3×30 mL) followed by sat. NaHCO₃ solution (3×30 mL). Organic layer was dried over anhydrous Na₂SO₄, filtered and concentrated to get crude compound. The crude compound was purified by prep HPLC to afford 2-(3-chlorophenyl)-2-methyl-1-phenylpropyl ((2S)-1-((4-(cyclopropylamino)-1-(5,5-dimethyl-2-oxopyrrolidin-3-yl)-3,4-dioxobutan-2-yl)amino)-4-methyl-1-oxopentan-2-yl)carbamate (C163). TLC system: 10% Methanol in dichloromethane Rf: 0.5 LCMS (ESI): m/z 667.2 (M+H)⁺

### Example 130: Synthesis of Compound C164

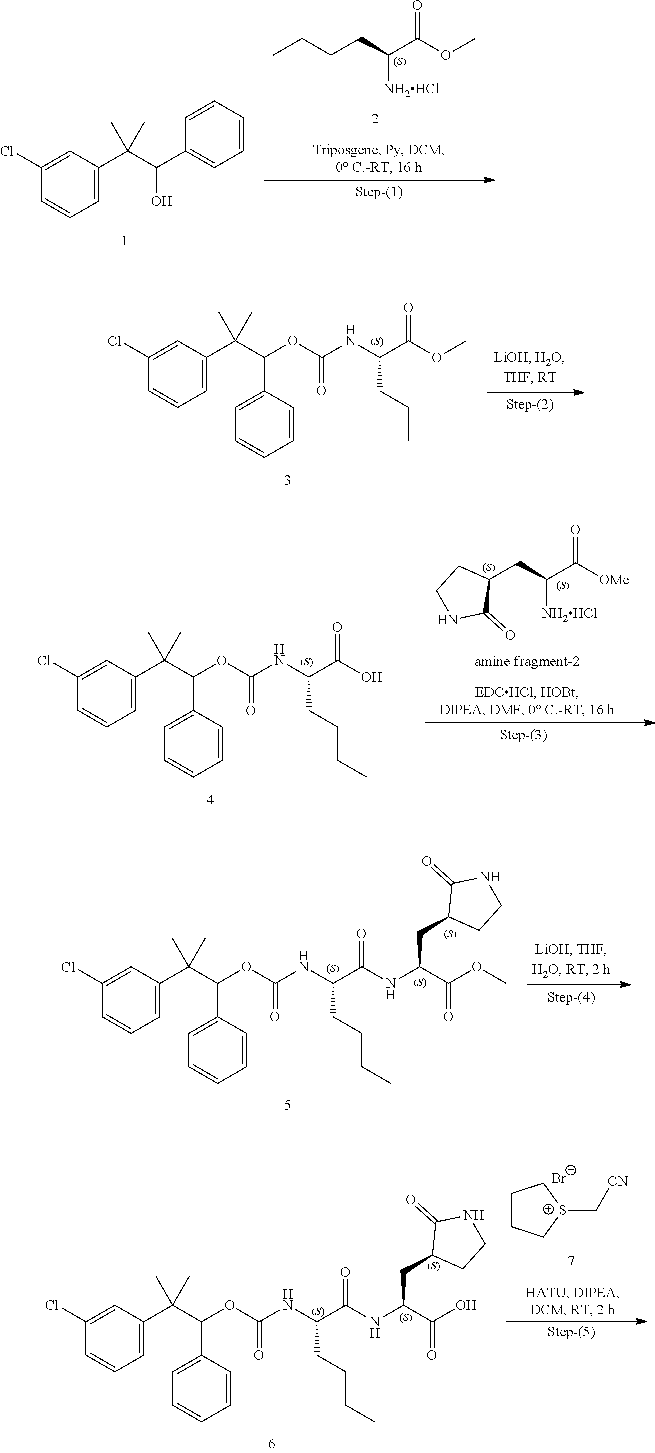

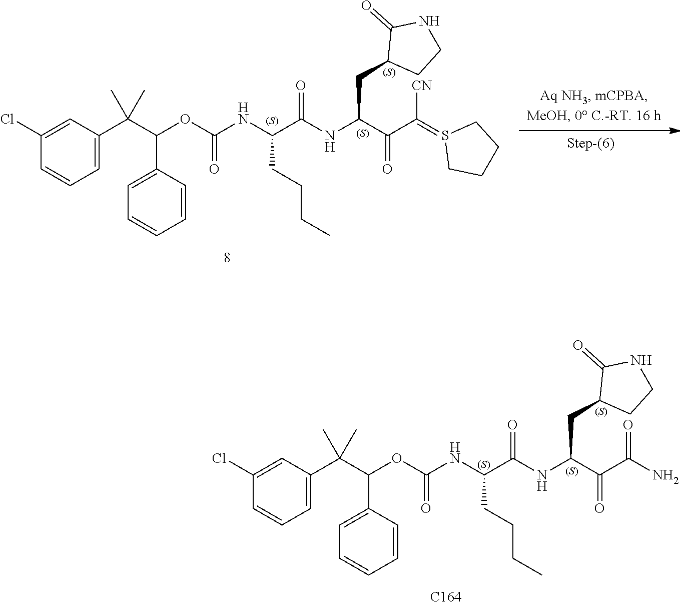

### Methyl (2S)-2-(((2-(3-chlorophenyl)-2-methyl-1-phenylpropoxy) carbonyl) amino) hexanoate (3)

To a stirred solution of 2-(3-chlorophenyl)-2-methyl-1-phenylpropan-1-ol (1) (2.5 g, 9.61 mmol) and methyl (S)-2-aminohexanoate hydrochloride (2) (2.0 g, 11.53 mmol) in DCM (40 mL) was added pyridine (7.5 mL, 3 vol) followed by triphosgene (1.4 g, 4.82 mmol) at 0° C. and stirred at room temperature for 16 h. The progress of the reaction was monitored by TLC and LCMS. Reaction mixture was diluted with DCM and washed with 1N HCl (50 mL), organic layer was dried over sodium sulfate, filtered and evaporated under reduced pressure. The crude residue was purified by combi-flash, compound eluted at 8% ethyl acetate in pet ether to afford methyl (2S)-2-(((2-(3-chlorophenyl)-2-methyl-1-phenylpropoxy) carbonyl) amino) hexanoate (3). TLC system: 10% Ethyl acetate in hexane Rf: 0.3 LCMS (ESI): m/z 432.32 [M+H]⁺

### (2S)-2-(((2-(3-Chlorophenyl)-2-methyl-1-phenylpropoxy) carbonyl) amino) hexanoic acid (4)

To a stirred solution of methyl (2S)-2-(((2-(3-chlorophenyl)-2-methyl-1-phenylpropoxy) carbonyl)amino)hexanoate (3) (3.0 g, 6.96 mmol) in THF (30 mL), water (10 mL) was added lithium hydroxide (500 mg, 20.88 mmol) at RT and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×50 mL), dried over sodium sulfate, concentrated under reduced pressure to afford (2S)-2-(((2-(3-chlorophenyl)-2-methyl-1-phenylpropoxy) carbonyl) amino) hexanoic acid (4). TLC system: 100% EtOAc Rf: 0.1 LCMS (ESI): m/z 416.31 [M−H]⁻

### Methyl (2S)-2-((2S)-2-(((2-(3-chlorophenyl)-2-methyl-1-phenylpropoxy) carbonyl) amino) hexanamido)-3-((S)-2-oxopyrrolidin-3-yl) propanoate (5)

To a stirred solution of (2S)-2-(((2-(3-chlorophenyl)-2-methyl-1-phenyl propoxy) carbonyl)amino)hexanoic acid (4) (2.5 g, 5.99 mmol) DMF (20 mL) added EDC.HCl (1.7 g, 8.98 mmol), HOBt (1.2 g, 8.98 mmol), DIPEA (3.0 mL, 17.97 mmol) and methyl (S)-2-amino-3-((S)-2-oxopyrrolidin-3-yl)propanoate hydrochloride (amine fragment-2) (1.3 g, 7.19 mmol) at 0° C. simultaneously and stirred at room temperature for 16 h. Reaction mixture was quenched with ice water (100 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% methanol in dichloromethane to afford methyl (2S)-2-((2S)-2-(((2-(3-chlorophenyl)-2-methyl-1-phenylpropoxy) carbonyl) amino) hexanamido)-3-((S)-2-oxopyrrolidin-3-yl) propanoate (5). TLC system: 10% Methanol in dichloromethane Rf: 0.4 LCMS (ESI): m/z 586.52 [M+H]⁺

### (2S)-2-((2S)-2-(((2-(3-Chlorophenyl)-2-methyl-1-phenylpropoxy) carbonyl) amino) hexanamido)-3-((S)-2-oxopyrrolidin-3-yl) propanoic acid (6)

To a stirred solution of methyl (2S)-2-((2S)-2-(((2-(3-chlorophenyl)-2-methyl-1-phenylpropoxy)carbonyl)amino)hexanamido)-3-((S)-2-oxopyrrolidin-3-yl)propanoate (5) (500 mg, 0.85 mmol) in THF (10 mL), water (3 mL) was added lithium hydroxide (61 mg, 2.56 mmol) at RT and stirred at room temperature for 2 h. The progress of the reaction was monitored by TLC and LCMS. Reaction mixture completely distilled under reduced pressure, crude compound acidified with aq. 1N HCl solution up to pH˜2 and extracted with ethyl acetate (2×30 mL), dried over sodium sulfate, concentrated under reduced pressure to afford (2S)-2-((2S)-2-(((2-(3-chlorophenyl)-2-methyl-1-phenylpropoxy)carbonyl)amino)hexanamido)-3-((S)-2-oxopyrrolidin-3-yl) propanoic acid (6). TLC system: 100% EtOAc Rf: 0.1 LCMS (ESI): m/z 572.50 [M+H]⁺

### 2-(3-Chlorophenyl)-2-methyl-1-phenylpropyl ((S)-1-(((S)-4-cyano-3-oxo-1-((S)-2-oxopyrrolidin-3-yl)-4-(tetrahydro-1l4-thiophen-1-ylidene) butan-2-yl) amino)-1-oxohexan-2-yl) carbamate (8)

To a stirred solution of (2S)-2-((2S)-2-(((2-(3-chlorophenyl)-2-methyl-1-phenylpropoxy) carbonyl)amino)hexanamido)-3-((S)-2-oxopyrrolidin-3-yl) propanoic acid (6) (350 mg, 0.612 mmol) DCM (10 mL) added HATU (465 mg, 1.22 mmol), DIPEA (0.3 mL, 1.91 mmol) and 1-(cyanomethyl) tetrahydro-1H-thiophen-1-iumbromide (7) (350 mg, 1.68 mmol) at 0° C. simultaneously and stirred at room temperature for 2 h. Reaction mixture was diluted with ice water (20 mL), extracted with dichloromethane (2×20 mL), dried over sodium sulfate and evaporated under reduced pressure. The crude residue was purified by combi-flash NP, compound eluted at 4% methanol in dichloromethane to afford 2-(3-chlorophenyl)-2-methyl-1-phenylpropyl ((S)-1-(((S)-4-cyano-3-oxo-1-((S)-2-oxopyrrolidin-3-yl)-4-(tetrahydro-1l4-thiophen-1-ylidene) butan-2-yl)amino)-1-oxohexan-2-yl) carbamate (8). TLC system: 10% Methanol in dichloromethane Rf: 0.3 LCMS (ESI): m/z 681.56 [M+H]⁺

### 2-(3-Chlorophenyl)-2-methyl-1-phenylpropyl ((S)-1-(((S)-4-amino-3,4-dioxo-1-((S)-2-oxopyrrolidin-3-yl) butan-2-yl) amino)-1-oxohexan-2-yl) carbamate (C164)

To a stirred solution of 2-(3-chlorophenyl)-2-methyl-1-phenylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-1-oxohexan-2-yl)carbamate (8) (200 mg, 0.29 mmol) in methanol (5 mL) was added m-CPBA (151 mg, 0.88 mmol) at 0° C. and the reaction mixture stirred for 2 h at 0° C. and added aq ammonia (2 mL) and stirred at RT for 16 h. The progress of the reaction was monitored by TLC and LCMS. Reaction mixture was quenched with sat. NaHCO₃ solution (40 mL) and extracted with DCM (2×20 mL). Organic layer was washed with brine solution (30 mL), dried over Na₂SO₄ and concentrated to get crude compound. The crude compound was purified by prep HPLC to afford 2-(3-chlorophenyl)-2-methyl-1-phenylpropyl ((S)-1-(((S)-4-amino-3,4-dioxo-1-((S)-2-oxopyrrolidin-3-yl) butan-2-yl)amino)-1-oxohexan-2-yl)carbamate (C164). TLC system: 10% Methanol in dichloromethane Rf: 0.4 LCMS (ESI): m/z 599.2 (M+H)⁺

### Example 131: Synthesis of Compound C166

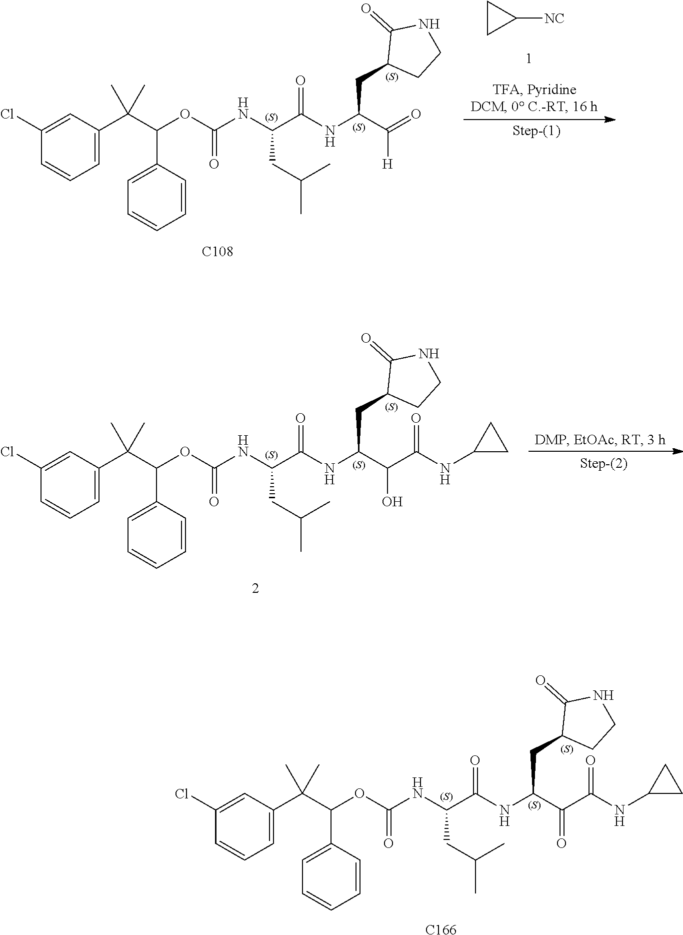

### 2-(3-Chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (2)

To a stirred solution of 2-(3-chlorophenyl)-2-methyl-1-phenylpropyl ((S)-4-methyl-1-oxo-1-(((S)-1-oxo-3-((S)-2-oxopyrrolidin-3-yl)propan-2-yl)amino)pentan-2-yl)carbamate (C108) (250 mg, 0.449 mmol) was dissolved in DCM (10 mL) added Pyridine (0.75 mL, 3 vol), isocyanocyclopropane (1) (45 mg, 0.671 mmol) sequentially at 0° C. and stirred for 10 min. To this was added TFA (0.06 mL, 899 mmol) at 0° C. and stirred at RT for 16 h. The progress of the reaction was monitored by TLC and LCMS. Reaction mixture was quenched with ice water (20 mL) and extracted with dichloromethane (2×15 mL). The organic layer was washed with 1N HCl (3×15 mL), brine solution (3×10 mL). The organic layer was dried over anhydrous Na₂SO₄ and evaporated under reduced pressure to afford crude 2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (2). TLC system: 5% Methanol/Dichloromethane Rf: 0.5 LCMS (ESI): m/z 641.5 [M+H]⁺

### 2-(3-Chlorophenyl)-2-methyl-1-phenylpropyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (C166)

To a stirred solution of 2-(3-chlorophenyl)-2-methyl-1-phenylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (2) (200 mg, 0.312 mmol) in EtOAc (10 mL) was added Dess-Martin periodinane (396 mg, 0.936 mmol) at 0° C. and stirred at RT for 3 h. The progress of the reaction was monitored by TLC and LCMS. Reaction mixture was filter through celite pad and washed with Ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford 2-(3-chlorophenyl)-2-methyl-1-phenylpropyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C166). TLC system: 10% Methanol/Dichloromethane Rf: 0.4 LCMS (ESI): m/z 639.2 (M+H)⁺

### Example 132: Synthesis of Compound C168

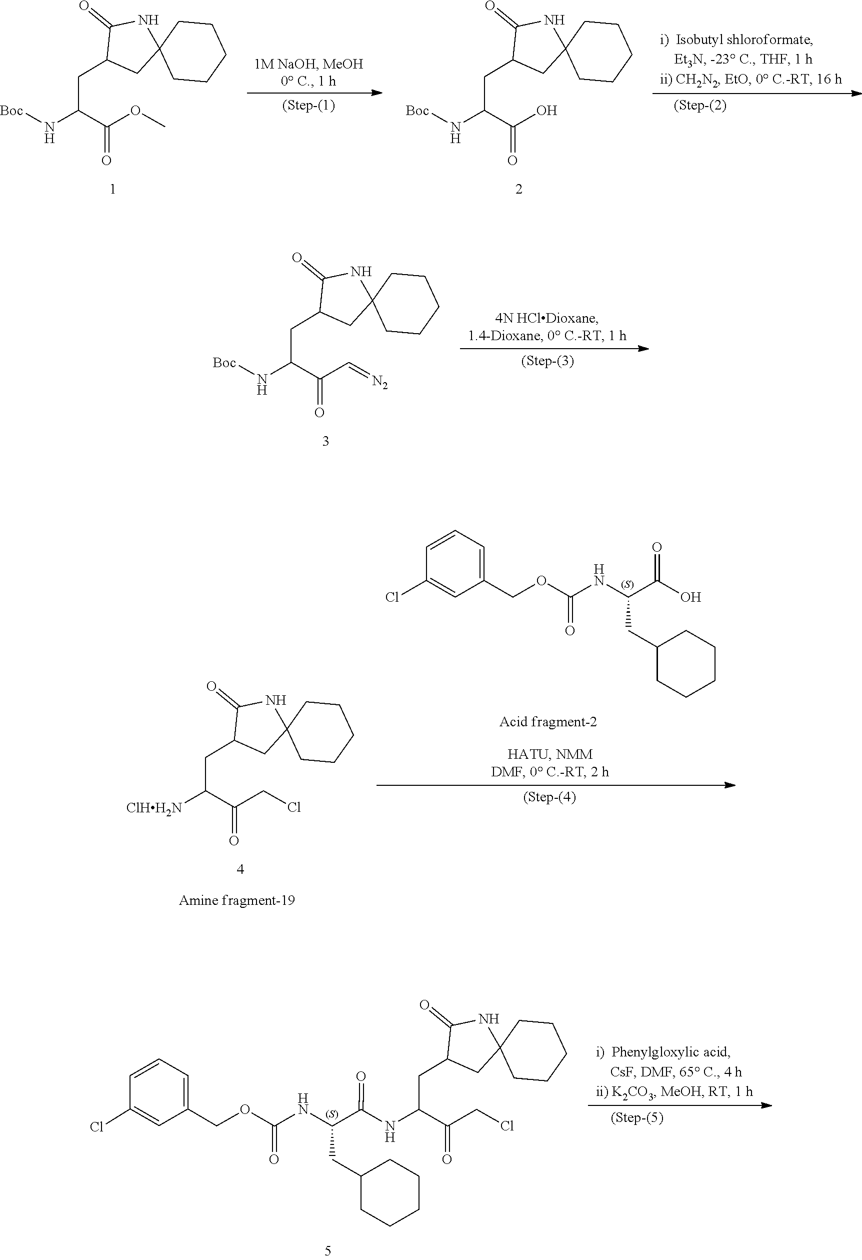

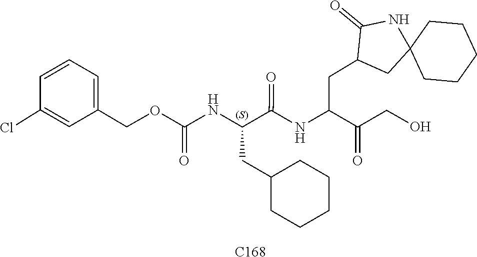

### (S)-2-((tert-Butoxycarbonyl)amino)-3-((S)-2-oxopyrrolidin-3-yl)propanoic acid (2)

A solution of methyl N-(tert-butoxycarbonyl)-3-[(3S)-2-oxopyrrolidin-3-yl]-L-alaninate (1) (1 g, 3.496 mmol) in methanol (10 mL) cooled to 0° C. then NaOH (500 mg in 5 mL water) solution was added. The resulting solution was stirred at 0° C. for 1 hour then removed the excess of methanol in vacuo. The residue was acidified to pH 3 and extracted with dichloromethane (2×50 ml), and the combined organics washed with brine, dried over MgSO4, concentrated under reduced pressure to afford (S)-2-((tert-butoxycarbonyl)amino)-3-((S)-2-oxopyrrolidin-3-yl)propanoic acid (2). TLC system: 50% Ethyl acetate in Pet ether R_{f}: 0.2

### tert-Butyl ((S)-4-diazo-3-oxo-1-((S)-2-oxopyrrolidin-3-yl)butan-2-yl)carbamate (3)

A solution of N-(tert-butoxycarbonyl)-3-[(3S)-2-oxopyrrolidin-3-yl]-L-alanine (3) (3 g, 11.0 29 mmol) in THF (30 mL) was placed under an atmosphere of N2 and cooled to −23° C. The resulting clear colorless solution was successively treated with triethylamine (3 mL, 22.059 mmol) followed by isobutylchloroformate (1.8 mL, 13.235 mmol). The reaction mixture was slowly treated with a solution of diazomethane (˜50 mL, −25 mmol) in diethyl ether. The resulting yellow clear solution was gradually warmed to RT and stirred for 1 h. Progress of the reaction was monitored by TLC and LCMS. After that the reaction mixture was quenched with sat. NaHCO₃ solution (50 mL), and extracted with ethyl acetate (100 mL), washed once with water (50 mL), once with brine (50 mL), dried over MgSO₄, filtered, and concentrated to give a. This material was purified by normal phase column chromatography to afford tert-Butyl ((S)-4-diazo-3-oxo-1-((S)-2-oxopyrrolidin-3-yl)butan-2-yl)carbamate (3). TLC system: 10% MeOH in DCM R_{f}: 0.5 LCMS (ESI): m/z 319.19 [M+Na]⁺

### (S)-3-((S)-2-Amino-4-chloro-3-oxobutyl)pyrrolidin-2-one hydrochloride (4)

A solution of tert-butyl ((1S)-3-diazo-2-oxo-1-{[(3S)-2-oxopyrrolidin-3-yl]methyl}propyl) carbamate (1.5 g, 5.06 mmol) in 1,4-dioxane (15 mL) was placed under an atmosphere of N2 and cooled to 0° C. This clear pale-yellow solution was drop-wise treated with a solution of 4M hydrochloric acid in 1,4-dioxane (15 mL). Upon complete addition, the reaction was warmed to RT over 1 h with the formation of white precipitate. Excess of 1,4-dioxane was evaporated under vacuum and the solid was triturated with diethyl ether to afford (S)-3-((S)-2-amino-4-chloro-3-oxobutyl)pyrrolidin-2-one hydrochloride (4) (1 g, 4.16 mmol, 83% yield) as a pale yellow solid. TLC system: 10% MeOH in DCM R_{f}: 0.1 LCMS (ESI): m/z 205.13 [M+H]⁺

### 3-Chlorobenzyl ((2S)-1-(((2S)-4-chloro-3-oxo-1-(2-oxo-1-azaspiro[4.5]decan-3-yl)butan-2-yl)amino)-3-cyclohexyl-1-oxopropan-2-yl)carbamate (5)

A solution of (S)-3-((S)-2-amino-4-chloro-3-oxobutyl)pyrrolidin-2-one hydrochloride (4) Amine fragment-19 (547 mg, 1.769 mmol) and (S)-2-((((3-chlorobenzyl)oxy)carbonyl)amino)-3-cyclohexylpropanoic acid Acid fragment-2 (500 mg, 1.474 mmol) and in DMF (50 mL) was placed under an atmosphere of N2 and cooled to 0° C. This pale-yellow solution was successively treated with HATU (841 mg, 2.212 mmol) and N-methylmorpholine (0.32 mL, 2.949 mmol). After 1 h, the reaction was quenched with 1:1 ice/sat NaHCO³ (50 mL) and extracted three times with ethyl acetate (50 mL). The combined organics were washed once with brine (100 mL), dried over MgSO4, filtered, and concentrated to give a yellow syrup. This material was purified by normal phase chromatography to afford 3-chlorobenzyl ((2S)-1-(((2S)-4-chloro-3-oxo-1-(2-oxo-1-azaspiro[4.5]decan-3-yl)butan-2-yl)amino)-3-cyclohexyl-1-oxopropan-2-yl)carbamate (5). TLC system: 10% MeOH in DCM R_{f}: 0.6 LCMS (ESI): m/z 594.47 [M+H]⁺

### 3-Chlorobenzyl ((2S)-3-cyclohexyl-1-(((2S)-4-hydroxy-3-oxo-1-(2-oxo-1-azaspiro[4.5]decan-3-yl)butan-2-yl)amino)-1-oxopropan-2-yl)carbamate (C168)

A solution of 3-chlorobenzyl ((2S)-1-(((2S)-4-chloro-3-oxo-1-(2-oxo-1-azaspiro[4.5]decan-3-yl)butan-2-yl)amino)-3-cyclohexyl-1-oxopropan-2-yl)carbamate (5) (300 mg, 0.42 mmol) and benzoylformic acid (95 mg, 0.63 mmol) in DMF (20 mL) was placed under an atmosphere of N2. This clear pale-yellow solution was treated with cesium fluoride (151 mg, 0.84 mmol) followed by heating to 65° C. After 4 hr, the now yellow suspension was cooled to RT, diluted with ethyl acetate (60 mL), washed three times water (30 mL), once with brine (30 mL), dried over MgSO4, filtered, and concentrated to give crude ((3S)-3-((S)-2-((((3-chlorobenzyl)oxy)carbonyl)amino)-3-cyclohexylpropanamido)-2-oxo-4-(2-oxo-1-azaspiro[4.5]decan-3-yl)butyl 2-oxo-2-phenylacetate as a crude yellow foam. MS (ESI+) for C38H4ClN3O8 m/z 707.3 (M+H)⁺. This crude was taken in methanol (40 mL) was placed under an atmosphere of N2 and treated with potassium carbonate (30 mg, 0.04 mmol) with vigorous stirring. After 1 hr the volatiles were removed in vacuo (bath <30° C.) to give a crude. This material was purified by normal phase chromatography to afford 3-chlorobenzyl ((2S)-3-cyclohexyl-1-(((2S)-4-hydroxy-3-oxo-1-(2-oxo-1-azaspiro[4.5]decan-3-yl)butan-2-yl)amino)-1-oxopropan-2-yl)carbamate (C168). TLC system: 10% MeOH in DCM R_{f}: 0.55 LCMS (ESI): m/z 576.2 [M+H]⁺

### Example 133: Synthesis of Compound C169

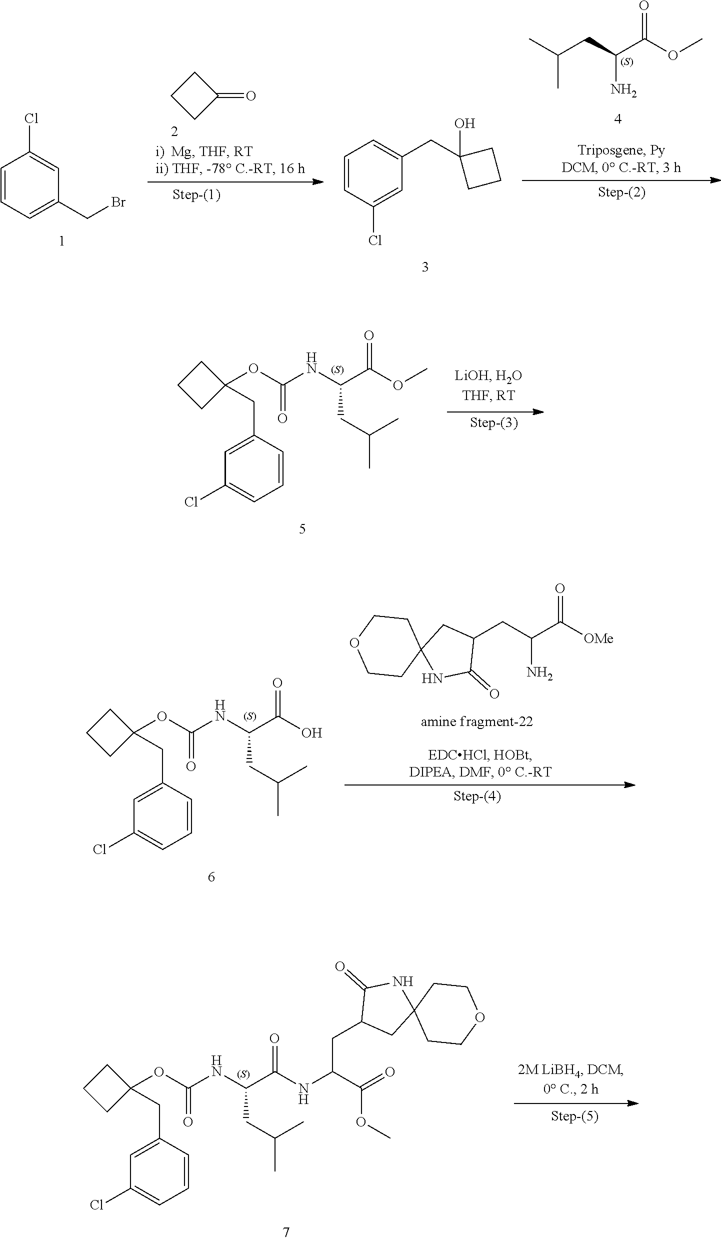

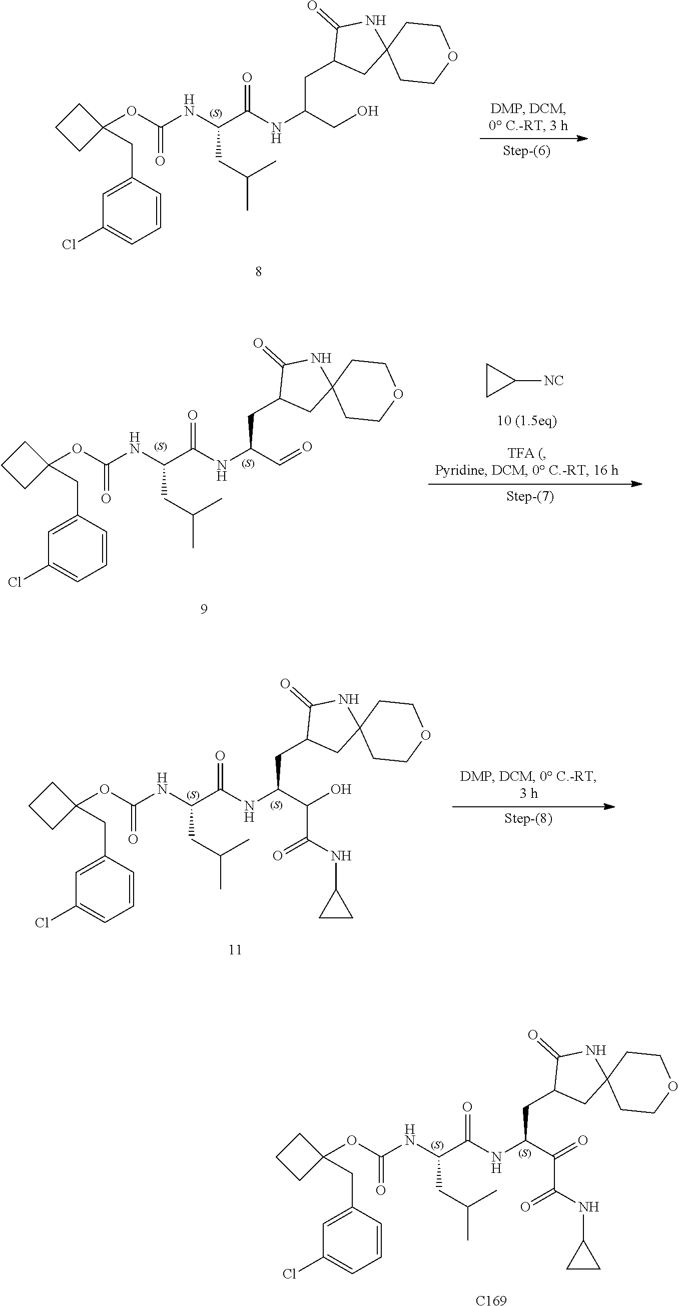

### 1-(3-chlorobenzyl)cyclobutan-1-ol (3)

To a stirred solution of 1-(bromomethyl)-3-chlorobenzene (1) (3 g, 14.705 mmol) in diethyl ether (60 mL) was added cat. 1,2-dibromoethane (0.2 mL, 1.47 mmol), cat. Iodine (94 mg, 0.73 mmol) and magnesium turnings (1 g, 44.11 mmol) at RT. Observed self-vigorous reflux. Maintained at RT for 1 h. Then the generated Grignard reagent was added to another RBF contained cyclobutanone (2) (1 g, 14.28 mmol) in diethyl ether (20 mL) slowly drop wise at −78° C. Maintained up to RT for 16 h. The progress of the reaction was monitored by TLC. The reaction mixture was quenched with saturated NH₄Cl (50 mL) and extracted with ethyl acetate (2×60 mL), dried over sodium sulfate and evaporated under reduced pressure. The crude residue was purified by silica gel column by eluting with 10% ethyl acetate in hexane to afford 1-(3-chlorobenzyl)cyclobutan-1-ol (3). TLC system: 10% Ethyl acetate in hexane Rf: 0.3 LCMS (ESI): m/z=197.28 [M+H]

### Methyl ((1-(3-chlorobenzyl)cyclobutoxy)carbonyl)-L-leucinate (5)

To a stirred solution of 1-(3-chlorobenzyl)cyclobutan-1-ol (3) (1.1 g, 5.61 mmol) in DCM (20 mL) was added pyridine (1.3 mL, 16.83 mmol) and methyl L-leucinate (4) (1.2 g, 8.41 mmol) followed by triphosgene (0.83 g, 2.806 mmol) at 0° C. with portion wise and stirred at RT for 16 h. The progress of the reaction was monitored by TLC. Reaction mixture was quenched with 1N aq. HCl (20 mL) then extracted with DCM (2×50 mL), washed with sat. NaHCO₃ solution (2×50 mL), dried over sodium sulfate and evaporated under reduced pressure. The crude residue was purified by silica gel column by eluting with 15% ethyl acetate in hexane to afford methyl ((1-(3-chlorobenzyl)cyclobutoxy)carbonyl)-L-leucinate (5). TLC system: 10% Ethyl acetate in hexane Rf: 0.4 LCMS (ESI): m/z=368.35 [M+H]⁺

### ((1-(3-Chlorobenzyl)cyclobutoxy)carbonyl)-L-leucine (6)

To a stirred solution of methyl ((1-(3-chlorobenzyl)cyclobutoxy)carbonyl)-L-leucinate (5) (1.6 g, 4.35 mmol) in THF (6.5 mL), water (1.6 mL) was added LiOH.H₂O (0.42 g, 17.43 mmol) at 0° C. Reaction mixture was stirred at RT for 3 h. The progress of the reaction was monitored by TLC. After consumption of starting material, the reaction mixture was concentrated and acidified with 1N HCl, extracted with ethyl acetate (2×50 mL), dried over sodium sulfate and evaporated under reduced pressure to afford ((1-(3-chlorobenzyl)cyclobutoxy)carbonyl)-L-leucine (6). TLC system: 10% Methanol in dichloromethane Rf: 0.2 LCMS (ESI): m/z=354.34 [M+H]⁺

### Methyl 2-((S)-2-(((1-(3-chlorobenzyl)cyclobutoxy)carbonyl)amino)-4-methylpentanamido)-3-(2-oxo-8-oxa-1-azaspiro[4.5]decan-3-yl)propanoate (7)

To a stirred solution of ((1-(3-chlorobenzyl)cyclobutoxy)carbonyl)-L-leucine (6) (1 g, 2.83 mmol) in DMF (20 mL) was added EDC.HCl (0.65 g, 4.24 mmol), HOBt (0.57 g, 4.22 mmol), DIPEA (1.2 mL, 8.49 mmol) and methyl 2-amino-3-(2-oxo-8-oxa-1-azaspiro[4.5]decan-3-yl)propanoate (amine-fragment-22) (1 g, 3.39 mmol) at 0° C. simultaneously and stirred at room temperature for 16 h. Reaction mixture was diluted with ice water (50 mL), extracted with ethyl acetate (2×50 mL), the organic layer was dried over sodium sulfate and evaporated under reduced pressure to afford crude compound. The crude residue was purified by silica gel column by eluting with 80% ethyl acetate and pet-ether to afford methyl 2-((S)-2-(((1-(3-chlorobenzyl)cyclobutoxy)carbonyl)amino)-4-methylpentanamido)-3-(2-oxo-8-oxa-1-azaspiro[4.5]decan-3-yl)propanoate (7). TLC system: 10% Methanol in dichloromethane Rf: 0.3 LCMS (ESI): m/z=592.53 [M+H]⁺

### 1-(3-Chlorobenzyl)cyclobutyl ((2S)-1-((1-hydroxy-3-(2-oxo-8-oxa-1-azaspiro[4.5]decan-3-yl)propan-2-yl)amino)-4-methyl-1-oxopentan-2-yl)carbamate (8)

To a stirred solution methyl 2-((S)-2-(((1-(3-chlorobenzyl)cyclobutoxy)carbonyl)amino)-4-methylpentanamido)-3-(2-oxo-8-oxa-1-azaspiro[4.5]decan-3-yl)propanoate (7) (800 mg, 1.35 mmol) in DCM (15 mL) was added 2M LiBH₄ in THF (1.3 mL, 2.7 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to get compound to afford 1-(3-chlorobenzyl)cyclobutyl ((2S)-1-((1-hydroxy-3-(2-oxo-8-oxa-1-azaspiro[4.5]decan-3-yl)propan-2-yl)amino)-4-methyl-1-oxopentan-2-yl)carbamate (8). TLC system: 10% Methanol in DCM Rf: 0.2 LCMS (ESI): m/z 564.52 (M+H)⁺

### 1-(3-chlorobenzyl)cyclobutyl ((2S)-4-methyl-1-oxo-1-((1-oxo-3-(2-oxo-8-oxa-1-azaspiro[4.5]decan-3-yl)propan-2-yl)amino)pentan-2-yl)carbamate (9)

To a stirred solution of 1-(3-chlorobenzyl)cyclobutyl ((2S)-1-((1-hydroxy-3-(2-oxo-8-oxa-1-azaspiro[4.5]decan-3-yl)propan-2-yl)amino)-4-methyl-1-oxopentan-2-yl)carbamate (8) (700 mg, 1.24 mmol) was dissolved in DCM (6 mL) was added Dess-Martin periodinane (1.05 mg, 2.48 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by normal phase purification afforded 1-(3-chlorobenzyl)cyclobutyl ((2S)-4-methyl-1-oxo-1-((1-oxo-3-(2-oxo-8-oxa-1-azaspiro[4.5]decan-3-yl)propan-2-yl)amino)pentan-2-yl)carbamate (9). TLC system: 10% Methanol in DCM Rf: 0.3 LCMS (ESI): m/z 562.32 (M+H)⁺

### 1-(3-Chlorobenzyl)cyclobutyl ((2S)-1-(((2S)-4-(cyclopropylamino)-3-hydroxy-4-oxo-1-(2-oxo-8-oxa-1-azaspiro[4.5]decan-3-yl)butan-2-yl)amino)-4-methyl-1-oxopentan-2-yl)carbamate (11)

To a stirred solution of afforded 1-(3-chlorobenzyl)cyclobutyl ((2S)-4-methyl-1-oxo-1-((1-oxo-3-(2-oxo-8-oxa-1-azaspiro[4.5]decan-3-yl)propan-2-yl)amino)pentan-2-yl)carbamate (9) (110 mg, 0.19 mmol) in DCM (5 mL) was added isocyanocyclopropane (10) (25 mg, 0.29 mmol), pyridine (0.7 mL, 0.78 mmol) at 0° C. and the reaction mixture was stirred for 30 minutes at 0° C. and added TFA (45 mg, 0.39 mmol) and stirred at RT for 16 h. The progress of the reaction was monitored by TLC and LCMS. Reaction mixture was acidified with 1N HCl solution (5 mL) and extracted with DCM (2×10 mL). Organic layer was washed with brine solution (5 mL), dried over Na₂SO₄ and concentrated to get crude compound. The crude compound to afford 1-(3-chlorobenzyl)cyclobutyl ((2S)-1-((4-(cyclopropylamino)-3-hydroxy-4-oxo-1-(2-oxo-8-oxa-1-azaspiro[4.5]decan-3-yl)butan-2-yl)amino)-4-methyl-1-oxopentan-2-yl)carbamate (11). TLC system: 10% Methanol in dichloromethane Rf: 0.2 LCMS (ESI): m/z 647.36 [M+H]⁺

### 1-(3-chlorobenzyl)cyclobutyl ((2S)-1-((4-(cyclopropylamino)-3,4-dioxo-1-(2-oxo-8-oxa-1-azaspiro[4.5]decan-3-yl)butan-2-yl)amino)-4-methyl-1-oxopentan-2-yl)carbamate (C169)

To a stirred solution of 1-(3-chlorobenzyl)cyclobutyl ((2S)-1-((4-(cyclopropylamino)-3-hydroxy-4-oxo-1-(2-oxo-8-oxa-1-azaspiro[4.5]decan-3-yl)butan-2-yl)amino)-4-methyl-1-oxopentan-2-yl)carbamate (11) (100 mg, 0.155 mmol) was dissolved in DCM (6 mL) was added Dess-Martin periodinane (262 mg, 0.62 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by normal phase purification afforded 1-(3-chlorobenzyl)cyclobutyl ((2S)-1-(((2S)-4-(cyclopropylamino)-3,4-dioxo-1-(2-oxo-8-oxa-1-azaspiro[4.5]decan-3-yl)butan-2-yl)amino)-4-methyl-1-oxopentan-2-yl)carbamate (C169). TLC system: 10% Methanol in DCM Rf: 0.3 LCMS (ESI): m/z 645.2 (M+H)⁺

### Example 134: Synthesis of Compounds C170 and C178

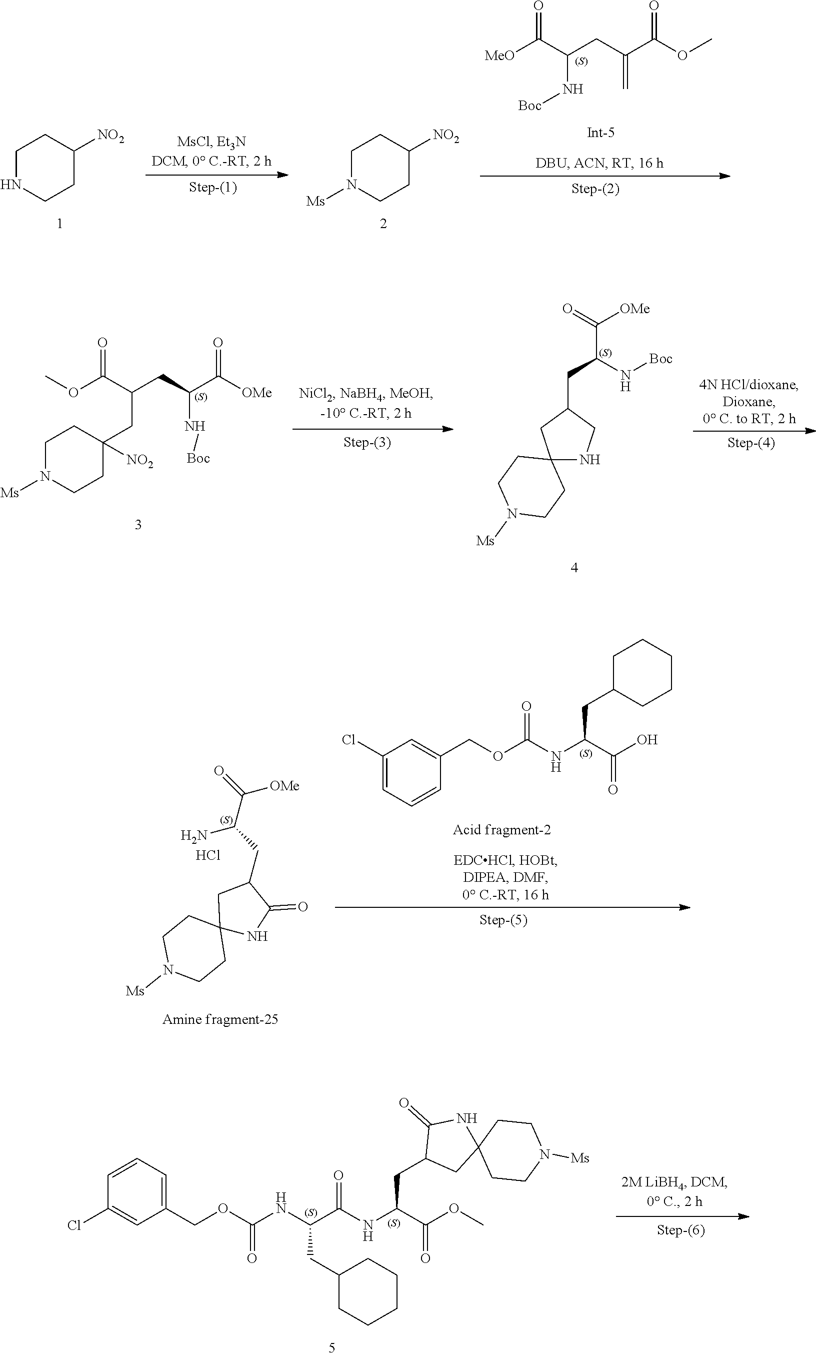

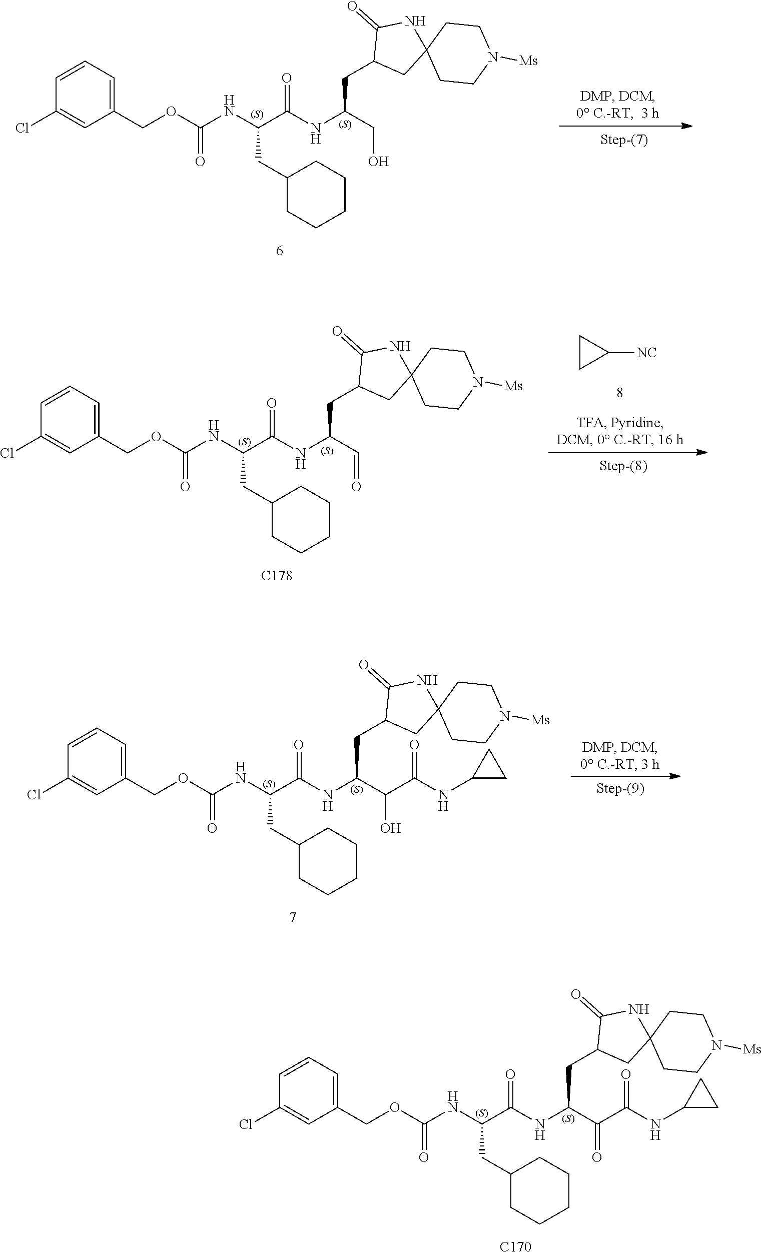

### 1-(Methylsulfonyl)-4-nitropiperidine (2)

To a stirred solution of 4-nitropiperidine hydrochloride (1) (3 g, 18 mmol) was dissolved in DCM (30 mL) was added mesyl chloride (158 mL, 19.87 mmol) and triethyl amine (3.79 mL, 27 mmol) at 0° C. simultaneously and stirred at RT for 2 h. The progress of the reaction was monitored by TLC and LCMS. Reaction mixture was quenched with ice water (40 mL) extracted with DCM (2×30 mL), organic layers were washed with water (2×20 ml), brine solution (20 mL), dried over sodium sulfate and evaporated under reduced pressure. The crude residue was purified by normal phase chromatography to afford 1-(methylsulfonyl)-4-nitropiperidine (2). TLC system: 30% EtOAc in pet ether Rf: 0.3 LCMS (ESI): m/z 209.14 [M+H]⁺

### Dimethyl 2-((1-acetyl-4-nitropiperidin-4-yl)methyl)-4-((tert-butoxycarbonyl)amino)pentanedioate (3)

To a stirred solution of dimethyl (S)-2-((tert-butoxycarbonyl)amino)-4-methylenepentanedioate (Int-5) (E0574-200) (3.03 g, 10.09 mmol) in ACN (20 mL) was added 1-(methylsulfonyl)-4-nitropiperidine (2) (1.5 g, 7.211 mmol) and DBU (2.19 mL, 14.42 mmol) at 0° C. and stirred at RT for 16 h. The progress of the reaction was monitored by TLC and LCMS. Reaction mixture was evaporated under reduced pressure. The crude residue was purified by normal phase chromatography to afford dimethyl 2-((1-acetyl-4-nitropiperidin-4-yl)methyl)-4-((tert-butoxycarbonyl)amino)pentanedioate (3). TLC system: 70% EtOAc in Pet ether R_{f}: 0.4 LCMS (ESI): m/z 496.8 (M+H)⁺

### Methyl (2S)-2-((tert-butoxycarbonyl)amino)-3-(8-(methylsulfonyl)-2-oxo-1,8-diazaspiro[4.5]decan-3-yl)propanoate (4)

To a stirred solution of dimethyl dimethyl (2S)-2-((tert-butoxycarbonyl)amino)-4-((1-(methylsulfonyl)-4-nitropiperidin-4-yl)methyl)pentanedioate (3) (2.8 g, 5.65 mmol) in methanol (30 mL) was added nickel chloride (801 mg, 6.21 mmol), followed by sodium borohydride (1.07 g, 28.28 mmol) at −10° C. and stirred at RT for 2 h. The progress of the reaction was monitored by TLC and LCMS. The reaction mixture was quenched with sat. ammonium chloride (50 mL) and extracted with 10% MeOH IN DCM (3×25 mL), combined organic layers were washed with water (2×20 ml), brine solution (25 mL), dried over sodium sulfate and evaporated under reduced pressure. The crude residue was purified by normal phase chromatography to afford methyl (2S)-2-((tert-butoxycarbonyl)amino)-3-(8-(methylsulfonyl)-2-oxo-1,8-diazaspiro[4.5]decan-3-yl)propanoate (4). TLC system: 10% MeOH/DCM R_{f}: 0.2 LCMS (ESI): m/z 378.36 (M+Na)⁺

### Methyl (2S)-2-amino-3-(8-(methylsulfonyl)-2-oxo-1,8-diazaspiro[4.5]decan-3-yl)propanoate hydrochloride (Amine fragment-25)

To a stirred solution of methyl (2S)-2-((tert-butoxycarbonyl)amino)-3-(8-(methylsulfonyl)-2-oxo-1,8-diazaspiro[4.5]decan-3-yl)propanoate (4) (2 g, 4.61 mmol) in 1,4-dioxane (20 mL) was added 4M HCl in 1,4-dioxane (10 mL) at 0° C. and stirred at RT for 2 h. The progress of the reaction was monitored by TLC. Reaction mixture was evaporated under reduced pressure. The crude residue was trituration with n-pentane to afford methyl (2S)-2-amino-3-(8-(methylsulfonyl)-2-oxo-1,8-diazaspiro[4.5]decan-3-yl)propanoate hydrochloride (Amine fragment-25). TLC system: 15% MeOH/DCM R_{f}: 0.1 LCMS (ESI): m/z 334.29 (M+H)⁺

### Methyl 2-((S)-2-((((3-chlorobenzyl)oxy)carbonyl)amino)-3-cyclohexylpropanamido)-3-(8-(methylsulfonyl)-2-oxo-1,8-diazaspiro[4.5]decan-3-yl)propanoate (5)

To a stirred solution of (S)-2-((((3-chlorobenzyl)oxy)carbonyl)amino)-3-cyclohexylpropanoic acid (Acid fragment-2) (1.3 g, 3.83 mmol) in DMF (20 mL), was added EDC.HCl (1.09 g, 5.75 mmol), HOBt (0.776 g, 5.751 mmol), DIPEA (2.0 mL, 11.502 mmol) and methyl (2S)-2-amino-3-(8-(methylsulfonyl)-2-oxo-1,8-diazaspiro[4.5]decan-3-yl)propanoate hydrochloride (Amine fragment-25) (1.55 g, 4.218 mmol) at 0° C. subsequently and stirred at room temperature for 16 h. Reaction mixture was diluted with ice water (20 mL), extracted with ethyl acetate (2×60 mL), dried over sodium sulfate and evaporated under reduced pressure. The crude residue was purified by combi-flash NP by eluting at 5% methanol in dichloromethane to methyl 2-((S)-2-((((3-chlorobenzyl)oxy)carbonyl)amino)-3-cyclohexylpropanamido)-3-(8-(methylsulfonyl)-2-oxo-1,8-diazaspiro[4.5]decan-3-yl)propanoate (5). TLC system: 5% Methanol in DCM Rf: 0.5 LCMS (ESI): m/z 655.41 [M+H]⁺

### 3-Chlorobenzyl ((2S)-3-cyclohexyl-1-(((2S)-1-hydroxy-3-(8-(methylsulfonyl)-2-oxo-1,8-diazaspiro[4.5]decan-3-yl)propan-2-yl)amino)-1-oxopropan-2-yl)carbamate (6)

To a stirred solution of methyl 2-((S)-2-((((3-chlorobenzyl)oxy)carbonyl)amino)-3-cyclohexylpropanamido)-3-(8-(methylsulfonyl)-2-oxo-1,8-diazaspiro[4.5]decan-3-yl)propanoate (5) (1.6 g, 2.4 mmol) in THF (20 mL), 2M LiBH₄ (2.4 mL, 4.8 mmol) at 0° C. and continued stirring for 3 h. The progress of the reaction was monitored by TLC and LCMS. Reaction mixture was quenched with sat. Ammonium chloride solution (10 mL) and extracted with ethyl acetate (2×10 mL), dried over sodium sulfate, concentrated under reduced pressure to afford 3-chlorobenzyl ((2S)-3-cyclohexyl-1-(((2S)-1-hydroxy-3-(8-(methylsulfonyl)-2-oxo-1,8-diazaspiro[4.5]decan-3-yl)propan-2-yl)amino)-1-oxopropan-2-yl)carbamate (6). TLC system: 5% Methanol in DCM Rf: 0.2 LCMS (ESI): m/z 627.40[M+H]⁺

### 3-Chlorobenzyl ((2S)-3-cyclohexyl-1-(((2S)-1-(8-(methylsulfonyl)-2-oxo-1,8-diazaspiro[4.5]decan-3-yl)-3-oxopropan-2-yl)amino)-1-oxopropan-2-yl)carbamate (C178)

At 0° C., to a stirred solution of 3-chlorobenzyl ((2S)-3-cyclohexyl-1-(((2S)-1-hydroxy-3-(8-(methylsulfonyl)-2-oxo-1,8-diazaspiro[4.5]decan-3-yl)propan-2-yl)amino)-1-oxopropan-2-yl)carbamate (6) (270 mg, 0.431 mmol) in ethyl acetate (4 mL) was added Dess-Martin periodinane (457 g, 1.07 mmol) and stirred at RT for 3 h. The progress of the reaction was monitored by TLC and LCMS. After 3 h, the reaction mixture was diluted with ethyl acetate (15 mL) and washed with sat. NaHCO₃ solution (3×20 mL). The organic layer was washed with brine solution (20 mL) and dried over Na₂SO₄ and concentrated to get crude compound. Crude was purified by prep HPLC to afford 3-chlorobenzyl ((2S)-3-cyclohexyl-1-(((2S)-1-(8-(methylsulfonyl)-2-oxo-1,8-diazaspiro[4.5]decan-3-yl)-3-oxopropan-2-yl)amino)-1-oxopropan-2-yl)carbamate (C178). TLC system: 10% Methanol in dichloromethane Rf: 0.3 LCMS (ESI): m/z 625.1[M+H]⁺

### 3-chlorobenzyl ((2S)-3-cyclohexyl-1-(((2S)-4-(cyclopropylamino)-3-hydroxy-1-(8-(methylsulfonyl)-2-oxo-1,8-diazaspiro[4.5]decan-3-yl)-4-oxobutan-2-yl)amino)-1-oxopropan-2-yl)carbamate (8)

To a stirred solution of 3-chlorobenzyl ((2S)-3-cyclohexyl-1-(((2S)-1-(8-(methylsulfonyl)-2-oxo-1,8-diazaspiro[4.5]decan-3-yl)-3-oxopropan-2-yl)amino)-1-oxopropan-2-yl)carbamate (C178) (300 mg, 0.48 mmol) in DCM (5 mL) was added isocyanocyclopropane (6) (64.32 mg, 0.96 mmol), pyridine (0.15 ml 1.92 mmol) at 0° C. and the reaction mixture was stirred for 30 minutes at 0° C. and added TFA (109.4 mg) and stirred at RT for 16 h. The progress of the reaction was monitored by TLC and LCMS. Reaction mixture was acidified with 1N HCl solution (5 mL) and extracted with DCM (2×10 mL). Organic layer was washed with brine solution (5 mL), dried over Na₂SO₄ and concentrated to get crude compound. The crude compound to afford 3-chlorobenzyl ((2S)-3-cyclohexyl-1-(((2S)-4-(cyclopropylamino)-3-hydroxy-1-(8-(methylsulfonyl)-2-oxo-1,8-diazaspiro[4.5]decan-3-yl)-4-oxobutan-2-yl)amino)-1-oxopropan-2-yl)carbamate (8). TLC system: 10% Methanol in dichloromethane Rf: 0.3 LCMS (ESI): m/z 710.33 [M+H]⁺

### 3-Chlorobenzyl ((2S)-3-cyclohexyl-1-(((2S)-4-(cyclopropylamino)-1-(8-(methylsulfonyl)-2-oxo-1,8-diazaspiro[4.5]decan-3-yl)-3,4-dioxobutan-2-yl)amino)-1-oxopropan-2-yl)carbamate (C170)

At 0° C., to a stirred solution of 33-chlorobenzyl ((2S)-3-cyclohexyl-1-(((2S)-4-(cyclopropylamino)-3-hydroxy-1-(8-(methylsulfonyl)-2-oxo-1,8-diazaspiro[4.5]decan-3-yl)-4-oxobutan-2-yl)amino)-1-oxopropan-2-yl)carbamate (8) (330 mg, 0.466 mmol) in ethyl acetate (5 mL) was added Dess-Martin periodinane (494 mg, 1.16 mmol) and stirred at RT for 3 h. The progress of the reaction was monitored by TLC and LCMS. After 3 h, the reaction mixture was diluted with ethyl acetate (15 mL) and washed with sat. NaHCO₃ solution (3×20 mL). The organic layer was washed with brine solution (20 mL) and dried over Na₂SO₄ and concentrated to get crude compound. Crude was purified by prep HPLC to afford 3-chlorobenzyl ((2S)-3-cyclohexyl-1-(((2S)-4-(cyclopropylamino)-1-(8-(methylsulfonyl)-2-oxo-1,8-diazaspiro[4.5]decan-3-yl)-3,4-dioxobutan-2-yl)amino)-1-oxopropan-2-yl)carbamate (C170). TLC system: 10% Methanol in dichloromethane Rf: 0.5 LCMS (ESI): m/z 708.2 [M+H]⁺

### Example 135: Synthesis of Compound C171

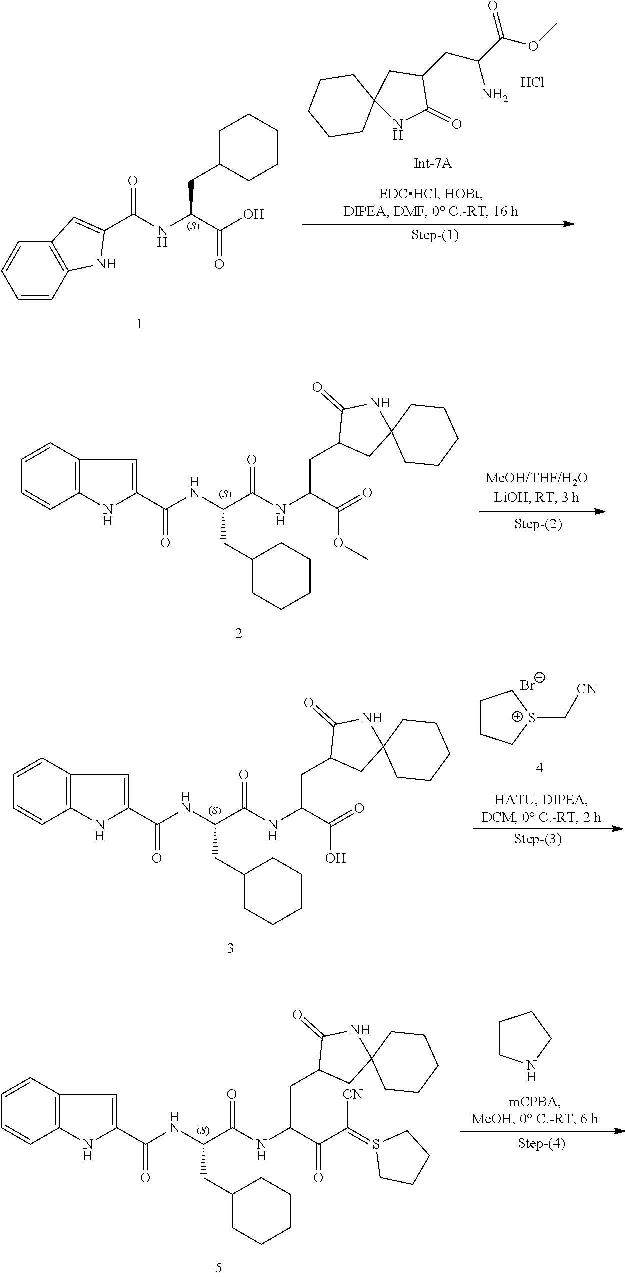

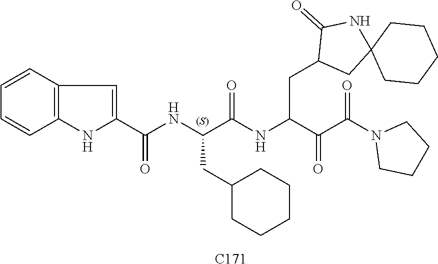

### Methyl 2-((S)-3-cyclohexyl-2-(1H-indole-2-carboxamido)propanamido)-3-(2-oxo-1-azaspiro[4.5]decan-3-yl)propanoate (2)

To a stirred solution of (S)-3-cyclohexyl-2-(1H-indole-2-carboxamido)propanoic acid (1) (Acid fragment-34) (1.5 g, 4.77 mmol) in DMF (30 mL) was added EDC.HCl (1.36 g, 7.165 mmol), HOBt (0.96 g, 7.165 mmol), DIPEA (2.54 mL, 14.31 mmol) and methyl-2-amino-3-(2-oxo-1-azaspiro[4.5]decan-3-yl)propanoate hydrochloride (Amine fragment-19) (1.68 g, 5.724 mmol) at 0° C., and stirred at room temperature for 16 h. Reaction mixture was quenched with ice water (250 mL) and extracted with ethyl acetate (2×100 mL). Combined the organic layer and washed with brine solution (2×200 mL), dried over sodium sulfate and evaporated under reduced pressure. The crude residue was purified by combi-flash NP, compound eluted at 10% methanol in dichloromethane to afford methyl 2-((S)-3-cyclohexyl-2-(1H-indole-2-carboxamido)propanamido)-3-(2-oxo-1-azaspiro[4.5]decan-3-yl)propanoate (2). TLC system: 10% Methanol in dichloromethane Rf: 0.45 LCMS (ESI): m/z 551.50 [M+H]⁺

### 2-((S)-3-cyclohexyl-2-(1H-indole-2-carboxamido)propanamido)-3-(2-oxo-1-azaspiro[4.5]decan-3-yl)propanoic acid (3)

To a stirred solution of methyl 2-((S)-3-cyclohexyl-2-(1H-indole-2-carboxamido)propanamido)-3-(2-oxo-1-azaspiro[4.5]decan-3-yl)propanoate (2) (1.5 g, 2.722 mmol) in THF (15 mL), water (15 mL) was added lithium hydroxide (343 mg, 8.166 mmol) at RT and stirred at room temperature for 2 h. The progress of the reaction was monitored by TLC and LCMS. Excess of THF was distilled under reduced pressure, crude compound acidified with aq. 1N HCl solution up to pH˜2 and extracted with DCM (2×50 mL), dried over sodium sulfate, concentrated under reduced pressure to afford 2-((S)-3-cyclohexyl-2-(1H-indole-2-carboxamido)propanamido)-3-(2-oxo-1-azaspiro[4.5]decan-3-yl)propanoic acid (3). TLC system: 10% Methanol in dichloromethane Rf: 0.1 LCMS (ESI): m/z 537.44 [M+H]⁺

### N-((2S)-1-((4-cyano-3-oxo-1-(2-oxo-1-azaspiro[4.5]decan-3-yl)-4-(tetrahydro-1λ⁴-thiophen-1-ylidene)butan-2-yl)amino)-3-cyclohexyl-1-oxopropan-2-yl)-1H-indole-2-carboxamide (5)

To a stirred solution of 2-((S)-3-cyclohexyl-2-(1H-indole-2-carboxamido)propanamido)-3-(2-oxo-1-azaspiro[4.5]decan-3-yl)propanoic acid (3) (1.3 g, 2.42 mmol), in DCM (25 mL) was added HATU (1.3 g, 3.358 mmol), DIPEA (1.2 mL, 6.716 mmol) and 1-(cyanomethyl)tetrahydro-1H-thiophen-1-ium (4) (556 mg, 2.686 mmol) at 0° C., and stirred at room temperature for 2 h. Reaction mixture was diluted with ice water (50 mL), extracted with DCM (2×50 mL). The combined organic layers were washed with water (1×50 mL), brine solution (1×50 mL), dried over sodium sulfate and evaporated under vacuum. The crude residue was purified by combi-flash NP, compound eluted at 5% methanol in dichloromethane to afford N-((2S)-1-((4-cyano-3-oxo-1-(2-oxo-1-azaspiro[4.5]decan-3-yl)-4-(tetrahydro-1λ⁴-thiophen-1-ylidene)butan-2-yl)amino)-3-cyclohexyl-1-oxopropan-2-yl)-1H-indole-2-carboxamide (5). TLC system: 10% Methanol in dichloromethane Rf: 0.45 LCMS (ESI): m/z 646.51 [M+H]⁺

### N-((2S)-3-cyclohexyl-1-((3,4-dioxo-1-(2-oxo-1-azaspiro[4.5]decan-3-yl)-4-(pyrrolidin-1-yl)butan-2-yl)amino)-1-oxopropan-2-yl)-1H-indole-2-carboxamide (C171)

To a stirred solution of N-((2S)-1-((4-cyano-3-oxo-1-(2-oxo-1-azaspiro[4.5]decan-3-yl)-4-(tetrahydro-1λ⁴-thiophen-1-ylidene)butan-2-yl)amino)-3-cyclohexyl-1-oxopropan-2-yl)-1H-indole-2-carboxamide (5) (200 mg, 0.310 mmol) in methanol (3 mL) was added m-CPBA (159 mg, 0.930 mmol) at 0° C. and the reaction mixture was stirred for 2 h at 0° C. and then added pyrrolidine (0.06 mL, 0.93 mmol) and stirred at RT for 16 h. The progress of the reaction was monitored by TLC and LCMS. Reaction mixture was quenched with sat. NaHCO₃ solution (40 mL) and extracted with DCM (2×15 mL). Organic layer was washed with water (1×20 mL) and brine solution (1×30 mL), dried over Na₂SO₄ and concentrated to get crude compound. The crude compound was purified by prep-HPLC to afford N-((2S)-3-cyclohexyl-1-((3,4-dioxo-1-(2-oxo-1-azaspiro[4.5]decan-3-yl)-4-(pyrrolidin-1-yl)butan-2-yl)amino)-1-oxopropan-2-yl)-1H-indole-2-carboxamide (C171). TLC system: 10% Methanol in dichloromethane Rf: 0.4 LCMS (ESI): m/z 616.4 [M+H]⁺

### Example 136: Synthesis of Compound C175

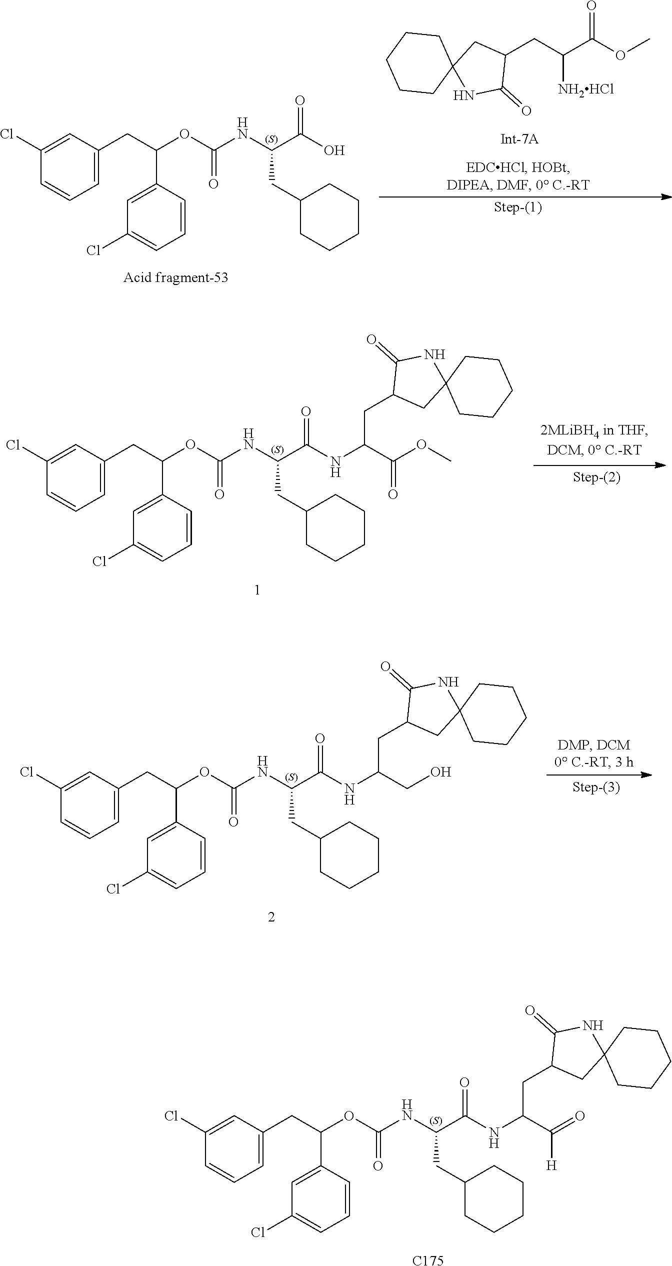

### Methyl 2-((2S)-2-(((1,2-bis(3-chlorophenyl)ethoxy)carbonyl)amino)-3-cyclohexylpropanamido)-3-(2-oxo-1-azaspiro[4.5]decan-3-yl)propanoate

To a stirred solution of (2S)-2-(((1,2-bis(3-chlorophenyl)ethoxy)carbonyl)amino)-3-cyclohexylpropanoic acid (acid fragment-53) (1 g, 2.07 mmol) in DMF (10 mL) was added EDC.HCl (596 mg, 3.11 mmol), HOBt (421 mg, 3.11 mmol), DIPEA (0.8 mL, 6.23 mmol) and methyl 2-amino-3-(2-oxo-1-azaspiro[4.5]decan-3-yl)propanoate hydrochloride (Int-7A) (603 mg, 2.07 mmol) at 0° C. simultaneously and stirred at room temperature for 16 h. Reaction mixture was quenched with ice water (80 mL) and extracted with ethyl acetate (2×50 mL). The organic layer was washed with brine solution (2×50 mL), dried over sodium sulfate and evaporated under reduced pressure. The crude residue was purified by combi-flash NP, compound eluted at 5% methanol in dichloromethane to afford methyl 2-((2S)-2-(((1,2-bis(3-chlorophenyl)ethoxy)carbonyl)amino)-3-cyclohexylpropanamido)-3-(2-oxo-1-azaspiro[4.5]decan-3-yl)propanoate (1). TLC system: 10% Methanol in dichloromethane Rf: 0.4 LCMS (ESI): m/z 700.31 [M+H]

### 1,2-Bis(3-chlorophenyl)ethyl ((2S)-3-cyclohexyl-1-((1-hydroxy-3-(2-oxo-1-azaspiro[4.5]decan-3-yl)propan-2-yl)amino)-1-oxopropan-2-yl)carbamate

To a stirred solution of methyl 2-((2S)-2-(((1,2-bis(3-chlorophenyl)ethoxy)carbonyl)amino)-3-cyclohexylpropanamido)-3-(2-oxo-1-azaspiro[4.5]decan-3-yl)propanoate (1) (900 mg, 1.28 mmol) in DCM (9 mL) was added 2M LiBH₄ in THF (1.28 mL, 2.57 mmol) at 0° C. and the reaction mixture stirred for 2 h at 0° C. The progress of the reaction was monitored by TLC and LCMS. Reaction mixture was quenched with sat. ammonium chloride solution (30 mL) and extracted with DCM (2×20 mL). Organic layer was washed with brine solution (30 mL), dried over Na₂SO₄ and concentrated to afford 1,2-bis(3-chlorophenyl)ethyl ((2S)-3-cyclohexyl-1-((1-hydroxy-3-(2-oxo-1-azaspiro[4.5]decan-3-yl)propan-2-yl)amino)-1-oxopropan-2-yl)carbamate (2). TLC system: 10% Methanol in dichloromethane Rf: 0.2 LCMS (ESI): m/z 672.50 [M+H]⁺

### 1,2-Bis(3-chlorophenyl)ethyl ((2S)-3-cyclohexyl-1-oxo-1-((1-oxo-3-(2-oxo-1-azaspiro[4.5]decan-3-yl)propan-2-yl)amino)propan-2-yl)carbamate (C175)

To a stirred solution of 1,2-bis(3-chlorophenyl)ethyl ((2S)-3-cyclohexyl-1-((1-hydroxy-3-(2-oxo-1-azaspiro[4.5]decan-3-yl)propan-2-yl)amino)-1-oxopropan-2-yl)carbamate (2) (800 mg, 1.190 mmol) was dissolved in DCM (10 mL) was added Dess-Martin periodinane (1 g, 2.38 mmol) at 0° C. and stirred at RT for 3 h. Reaction mixture was diluted with DCM (20 mL) and washed with sat. Hypo solution (3×30 mL) followed by sat. NaHCO₃ solution (3×30 mL). Organic layer was dried over anhydrous Na₂SO₄, filtered and concentrated to get crude compound. The crude compound was purified by combi-flash NP, compound eluted at 5% methanol in dichloromethane to afford 1,2-bis(3-chlorophenyl)ethyl ((2S)-3-cyclohexyl-1-oxo-1-((1-oxo-3-(2-oxo-1-azaspiro[4.5]decan-3-yl)propan-2-yl)amino)propan-2-yl)carbamate (C175). TLC system: 10% Methanol in dichloromethane Rf: 0.5 LCMS (ESI): m/z 670.2 (M+H)⁺

### Example 137: Synthesis of Compound C176 and C200

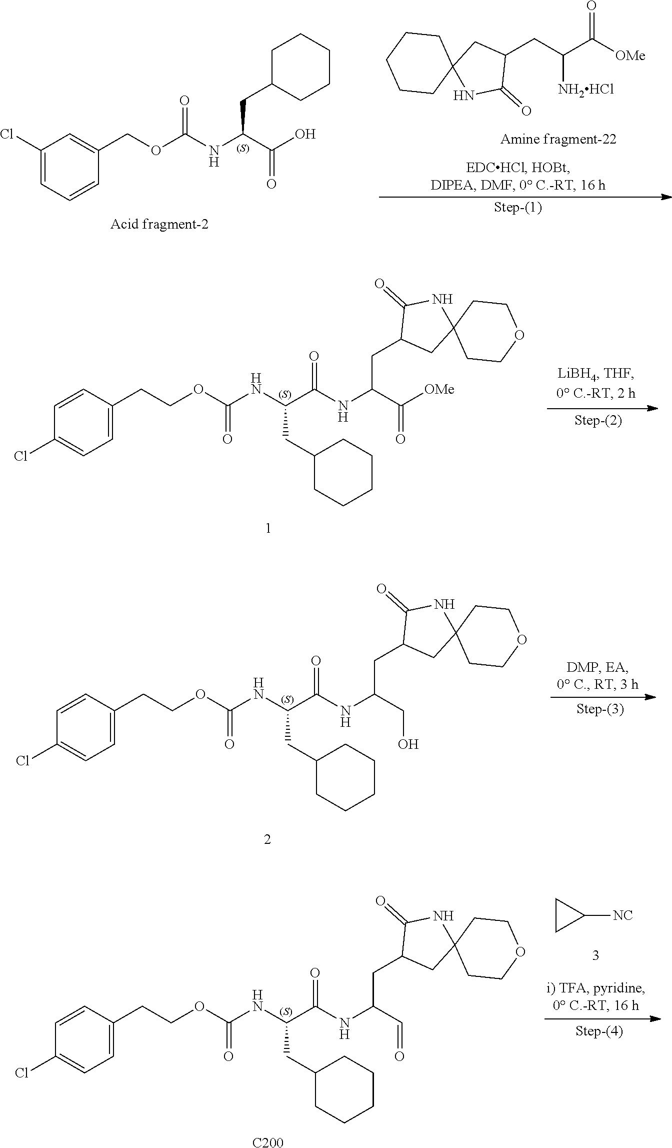

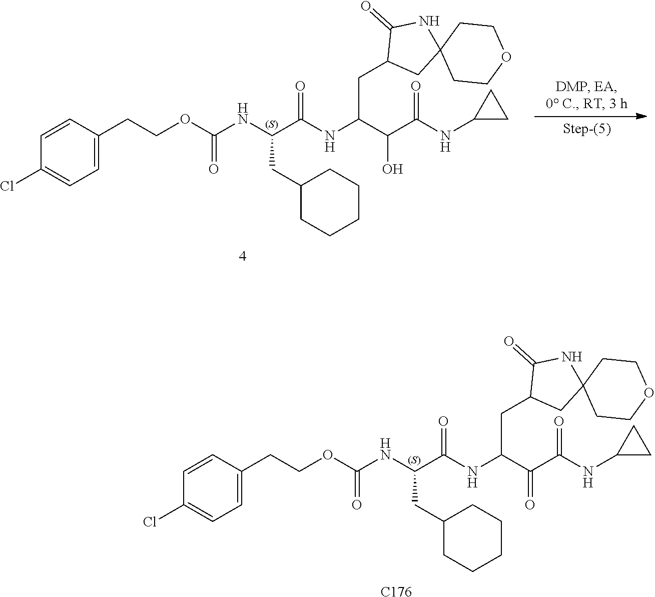

### Methyl 2-((S)-2-((((3-chlorobenzyl)oxy)carbonyl)amino)-3-cyclohexylpropanamido)-3-(2-oxo-8-oxa-1-azaspiro[4.5]decan-3-yl)propanoate (1)

To a stirred solution of (S)-2-((((3-chlorobenzyl)oxy)carbonyl)amino)-3-cyclohexylpropanoic acid (Acid fragment-2) (1.0 g, 2.94 mmol) in DMF (200 mL) was added EDC.HCl (842 mg, 4.41 mmol), HOBt (595 mg, 4.41 mmol), DIPEA (1.5 mL, 8.82 mmol) and methyl (2S)-2-amino-3-(2-oxo-8-oxa-1-azaspiro[4.5]decan-3-yl)propanoate hydrochloride (Amine fragment-22) (992 mg, 3.24 mmol) at 0° C., and stirred at room temperature for 16 h. Reaction mixture was quenched with ice water (50 mL) and extracted with ethyl acetate (2×25 mL). Combined the organic layer and washed with brine solution (2×20 mL), dried over sodium sulfate and evaporated under reduced pressure. The crude residue was purified by combi-flash NP, compound eluted at 5% methanol in dichloromethane to afford methyl 2-((S)-2-((((3-chlorobenzyl)oxy)carbonyl)amino)-3-cyclohexylpropanamido)-3-(2-oxo-8-oxa-1-azaspiro[4.5]decan-3-yl)propanoate (1). TLC system: 5% Methanol in dichloromethane Rf: 0.6 LCMS (ESI): m/z 578.28 [M+H]⁺

### 3-Chlorobenzyl ((2S)-3-cyclohexyl-1-((1-hydroxy-3-(2-oxo-8-oxa-1-azaspiro[4.5]decan-3-yl)propan-2-yl)amino)-1-oxopropan-2-yl)carbamate (2)

To a stirred solution of methyl (2S)-2-((S)-2-((((3-chlorobenzyl)oxy)carbonyl)amino)-3-cyclohexylpropanamido)-3-(2-oxo-8-oxa-1-azaspiro[4.5]decan-3-yl)propanoate (2) (1.6 g, 2.76 mmol) in THF (10 mL) at 0° C. was added 2M LiBH₄ in THF (2.7 mL, 5.53 mmol) then reaction mixture was stirred for 2 h at room temperature. Reaction mixture was quenched with sat. Ammonium chloride solution (20 mL) and extracted with EtoAc (2×10 mL). Organic layer was washed with brine solution, dried over Na₂SO₄ and concentrated to get crude to afford pure 3-chlorobenzyl ((2S)-3-cyclohexyl-1-((1-hydroxy-3-(2-oxo-8-oxa-1-azaspiro[4.5]decan-3-yl)propan-2-yl)amino)-1-oxopropan-2-yl)carbamate (2). TLC system: 5% Methanol in DCM Rf: 0.4 LCMS (ESI): m/z 550.31[M+H]⁺

### 3-Chlorobenzyl ((2S)-3-cyclohexyl-1-oxo-1-(((2S)-1-oxo-3-(2-oxo-8-oxa-1-azaspiro[4.5]decan-3-yl)propan-2-yl)amino)propan-2-yl)carbamate (C200)

To a stirred solution of 3-chlorobenzyl 3-chlorobenzyl ((2S)-3-cyclohexyl-1-(((2S)-1-hydroxy-3-(2-oxo-8-oxa-1-azaspiro[4.5]decan-3-yl)propan-2-yl)amino)-1-oxopropan-2-yl)carbamate (2) (300 mg, 0.545 mmol) in EtOAc (6 mL) was added Dess-Martin periodinane (578 mg, 1.36 mmol) at 0° C. and stirred at RT for 3 h. The progress of the reaction was monitored by TLC and LCMS. Reaction mixture was filter through celite pad and washed with EtoAc (20 mL) and filtrate was washed with hypo solution (3×10 mL) followed by saturated NaHCO₃ solution (3×10 mL). Organic layer was dried over anhydrous Na₂SO₄, filtered and concentrated to get crude, residue was purified by combi-flash NP, compound eluted at 10% methanol in dichloromethane afford to 3-chlorobenzyl ((2S)-3-cyclohexyl-1-oxo-1-(((2S)-1-oxo-3-(2-oxo-8-oxa-1-azaspiro[4.5]decan-3-yl)propan-2-yl)amino)propan-2-yl)carbamate (C200). TLC system: 5% Methanol in dichloromethane Rf: 0.3 LCMS (ESI): m/z 548.1 [M+H]⁺

### 3-Chlorobenzyl ((2S)-3-cyclohexyl-1-((4-(cyclopropylamino)-3-hydroxy-4-oxo-1-(2-oxo-8-oxa-1-azaspiro[4.5]decan-3-yl)butan-2-yl)amino)-1-oxopropan-2-yl)carbamate (4)

To a stirred solution of 3-chlorobenzyl ((2S)-3-cyclohexyl-1-oxo-1-(((2S)-1-oxo-3-(2-oxo-8-oxa-1-azaspiro[4.5]decan-3-yl)propan-2-yl)amino)propan-2-yl)carbamate (C200) (300 mg, 0.547 mmol) and isocyanocyclopropane (3) (73.3 mg, 1.09 mmol) was dissolved in DCM (10 mL), then added pyridine (0.17 mL), at 0° C. and stirred for 10 min. To this solution was added TFA (124 mg, 1.09 mmol) at 0° C. and stirred at RT for 16 h. The progress of the reaction was monitored by TLC and LCMS. Reaction mixture was quenched with ice water (10 mL) and extracted with dichloromethane (2×15 mL). The organic layer was washed with 1N HCl (3×5 mL), brine solution (3×5 mL). The organic layer was dried over anhydrous Na₂SO₄ and evaporated under reduced pressure to afford crude 3-chlorobenzyl ((2S)-3-cyclohexyl-1-(((2S)-4-(cyclopropylamino)-3-hydroxy-4-oxo-1-(2-oxo-8-oxa-1-azaspiro[4.5]decan-3-yl)butan-2-yl)amino)-1-oxopropan-2-yl)carbamate (4). TLC system: 10% Methanol in dichloromethane Rf: 0.5 LCMS (ESI): m/z 633.34 [M+H]⁺

### 3-Chlorobenzyl ((2S)-3-cyclohexyl-1-((4-(cyclopropylamino)-3,4-dioxo-1-(2-oxo-8-oxa-1-azaspiro[4.5]decan-3-yl)butan-2-yl)amino)-1-oxopropan-2-yl)carbamate (C176)

To a stirred solution of 3-chlorobenzyl ((2S)-3-cyclohexyl-1-(((2S)-4-(cyclopropylamino)-3-hydroxy-4-oxo-1-(2-oxo-8-oxa-1-azaspiro[4.5]decan-3-yl)butan-2-yl)amino)-1-oxopropan-2-yl)carbamate (4) (340 mg, 0.537 mmol) was added Dess-Martin periodinane (569 mg, 1.34 mmol) at 0° C. and stirred at RT for 3 h. The progress of the reaction was monitored by TLC and LCMS. Reaction mixture was filter through celite pad and washed with EtoAc (10 mL) and filtrate was washed with hypo solution (3×20 mL) followed by saturated NaHCO₃ solution (3×20 mL). Organic layer was dried over anhydrous Na₂SO₄, filtered and concentrated to get crude, residue was purified by combi-flash NP, compound eluted at 10% methanol in dichloromethane afford to 3-chlorobenzyl ((2S)-3-cyclohexyl-1-((4-(cyclopropylamino)-3,4-dioxo-1-(2-oxo-8-oxa-1-azaspiro[4.5]decan-3-yl)butan-2-yl)amino)-1-oxopropan-2-yl)carbamate (C176). TLC system: 10% Methanol in dichloromethane Rf: 0.4 LCMS (ESI): m/z 631.2 [M+H]⁺

### Example 138: Synthesis of Compounds C177 and C189

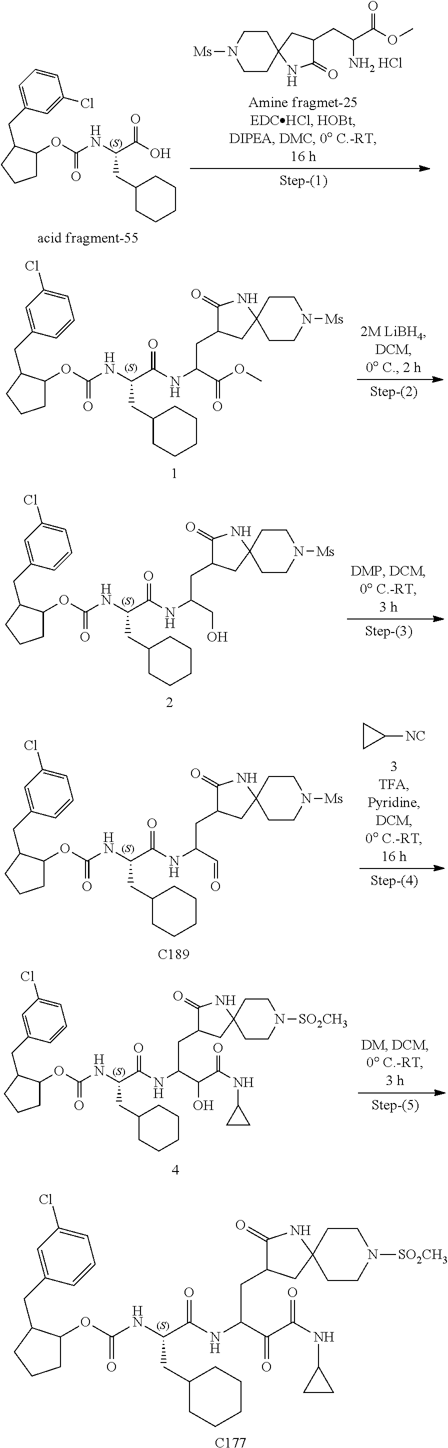

### Methyl 2-((2S)-2-((((2-(3-chlorobenzyl)cyclopentyl)oxy)carbonyl)amino)-3-cyclohexylpropanamido)-3-(8-(methylsulfonyl)-2-oxo-1,8-diazaspiro[4.5]decan-3-yl)propanoate (1)

To a stirred solution of (2S)-2-((((2-(3-chlorobenzyl)cyclopentyl)oxy)carbonyl)amino)-3-cyclohexylpropanoic acid (Acid fragment-55) (1 g, 2.44 mmol) in DMF (20 mL) was added EDC.HCl (0.69 g, 3.66 mmol), HOBt (0.49 g, 3.66 mmol), DIPEA (1.2 mL, 7.32 mmol) and methyl 2-amino-3-(8-(methylsulfonyl)-2-oxo-1,8-diazaspiro[4.5]decan-3-yl)propanoate hydrochloride (amine-fragment-25) (1 g, 3.39 mmol) at 0° C. simultaneously and stirred at room temperature for 16 h. Reaction mixture was diluted with ice water (50 mL), extracted with ethyl acetate (2×50 mL), the organic layer was dried over sodium sulfate and evaporated under reduced pressure to afford crude compound. The crude residue was purified by silica gel column by eluting with 80% ethyl acetate and pet-ether to afford methyl 2-((2S)-2-((((2-(3-chlorobenzyl)cyclopentyl)oxy)carbonyl)amino)-3-cyclohexylpropanamido)-3-(8-(methylsulfonyl)-2-oxo-1,8-diazaspiro[4.5]decan-3-yl)propanoate (1). TLC system: 10% Methanol in dichloromethane Rf: 0.3 LCMS (ESI): m/z=723.69 [M+H]⁺

### 2-(3-Chlorobenzyl)cyclopentyl ((2S)-3-cyclohexyl-1-((1-hydroxy-3-(8-(methylsulfonyl)-2-oxo-1,8-diazaspiro[4.5]decan-3-yl)propan-2-yl)amino)-1-oxopropan-2-yl)carbamate (2)

To a stirred solution methyl 2-((2S)-2-((((2-(3-chlorobenzyl)cyclopentyl)oxy)carbonyl)amino)-3-cyclohexylpropanamido)-3-(8-(methylsulfonyl)-2-oxo-1,8-diazaspiro[4.5]decan-3-yl)propanoate (1) (1.6 g, 2.21 mmol) in DCM (15 mL) was added 2M LiBH₄ in THF (2.2 mL, 4.43 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to get compound to 2-(3-chlorobenzyl)cyclopentyl ((2S)-3-cyclohexyl-1-((1-hydroxy-3-(8-(methylsulfonyl)-2-oxo-1,8-diazaspiro[4.5]decan-3-yl)propan-2-yl)amino)-1-oxopropan-2-yl)carbamate (2). TLC system: 10% Methanol in DCM Rf: 0.2 LCMS (ESI): m/z 695.40 (M+H)⁺

### 2-(3-Chlorobenzyl)cyclopentyl ((2S)-3-cyclohexyl-1-((1-(8-(methylsulfonyl)-2-oxo-1,8-diazaspiro[4.5]decan-3-yl)-3-oxopropan-2-yl)amino)-1-oxopropan-2-yl)carbamate (C189)

To a stirred solution of 2-(3-chlorobenzyl)cyclopentyl ((2S)-3-cyclohexyl-1-((1-hydroxy-3-(8-(methylsulfonyl)-2-oxo-1,8-diazaspiro[4.5]decan-3-yl)propan-2-yl)amino)-1-oxopropan-2-yl)carbamate (2) (150 mg, 0.22 mmol) was dissolved in DCM (4 mL) was added Dess-Martin periodinane (235 mg, 0.55 mmol) at 0° C. and stirred at RT for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by normal phase purification afforded 2-(3-chlorobenzyl)cyclopentyl ((2S)-3-cyclohexyl-1-((1-(8-(methylsulfonyl)-2-oxo-1,8-diazaspiro[4.5]decan-3-yl)-3-oxopropan-2-yl)amino)-1-oxopropan-2-yl)carbamate (C189). TLC system: 5% Methanol in DCM Rf: 0.3 LCMS (ESI): m/z 693.2 (M+H)⁺

### 2-(3-chlorobenzyl)cyclopentyl ((2S)-3-cyclohexyl-1-((4-(cyclopropylamino)-3-hydroxy-1-(8-(methylsulfonyl)-2-oxo-1,8-diazaspiro[4.5]decan-3-yl)-4-oxobutan-2-yl)amino)-1-oxopropan-2-yl)carbamate (4)

To a stirred solution of afforded 2-(3-chlorobenzyl)cyclopentyl ((2S)-3-cyclohexyl-1-((1-(8-(methylsulfonyl)-2-oxo-1,8-diazaspiro[4.5]decan-3-yl)-3-oxopropan-2-yl)amino)-1-oxopropan-2-yl)carbamate (C189) (380 mg, 0.54 mmol) in DCM (5 mL) was added isocyanocyclopropane (3) (74 mg, 1.09 mmol), pyridine (0.7 mL, 2.1 mmol) at 0° C. and the reaction mixture was stirred for 30 minutes at 0° C. and added TFA (0.12 ml, 1.09 mmol) and stirred at RT for 16 h. The progress of the reaction was monitored by TLC and LCMS. Reaction mixture was acidified with 1N HCl solution (5 mL) and extracted with DCM (2×10 mL). Organic layer was washed with brine solution (5 mL), dried over Na₂SO₄ and concentrated to get crude compound. The crude compound to afford 2-(3-chlorobenzyl)cyclopentyl ((2S)-3-cyclohexyl-1-((4-(cyclopropylamino)-3-hydroxy-1-(8-(methylsulfonyl)-2-oxo-1,8-diazaspiro[4.5]decan-3-yl)-4-oxobutan-2-yl)amino)-1-oxopropan-2-yl)carbamate (4). TLC system: 5% Methanol in dichloromethane Rf: 0.2 LCMS (ESI): m/z 778.81 [M+H]⁺

### 2-(3-chlorobenzyl)cyclopentyl ((2S)-3-cyclohexyl-1-((4-(cyclopropylamino)-1-(8-(methylsulfonyl)-2-oxo-1,8-diazaspiro[4.5]decan-3-yl)-3,4-dioxobutan-2-yl)amino)-1-oxopropan-2-yl)carbamate (C177)

To a stirred solution of 2-(3-chlorobenzyl)cyclopentyl ((2S)-3-cyclohexyl-1-((4-(cyclopropylamino)-3-hydroxy-1-(8-(methylsulfonyl)-2-oxo-1,8-diazaspiro[4.5]decan-3-yl)-4-oxobutan-2-yl)amino)-1-oxopropan-2-yl)carbamate (4) (330 mg, 1.06 mmol) was dissolved in DCM (6 mL) was added Dess-Martin periodinane (449 mg, 0.62 mmol) at 0° C. and stirred at RT for 3 h. Reaction mixture was diluted with dichloromethane (10 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by normal phase purification afforded 2-(3-chlorobenzyl)cyclopentyl ((2S)-3-cyclohexyl-1-((4-(cyclopropylamino)-1-(8-(methylsulfonyl)-2-oxo-1,8-diazaspiro[4.5]decan-3-yl)-3,4-dioxobutan-2-yl)amino)-1-oxopropan-2-yl)carbamate (C177). TLC system: 5% Methanol in DCM Rf: 0.3 LCMS (ESI): m/z 776.2 (M+H)⁺

### Example 139: Synthesis of Compound C179

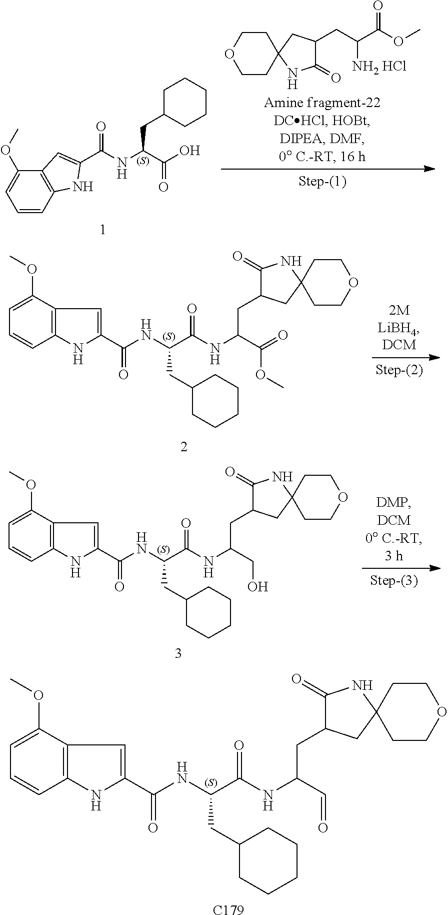

### Methyl 2-((S)-3-cyclohexyl-2-(4-methoxy-1H-indole-2-carboxamido)propanamido)-3-(2-oxo-8-oxa-1-azaspiro[4.5]decan-3-yl)propanoate (2)

To a stirred solution of (S)-3-cyclohexyl-2-(4-methoxy-1H-indole-2-carboxamido)propanoic acid (1) (1.0 g, 2.9 mmol) in DMF at 0° C. was added EDC.HCl (0.83 g, 4.35 mmol), HOBT (0.587 g, 4.35 mmol), DIPEA (1.6 mL, 3 Vol.) and methyl 2-amino-3-(2-oxo-8-oxa-1-azaspiro[4.5]decan-3-yl)propanoate hydrochloride (Amine fragment-22) (1.0 g, 3.4 mmol) simultaneously and stirred at room temperature for 16 h. The progress of the reaction was monitored by TLC and LCMS. After 16 h, reaction mixture was quenched with ice water (20 mL), extracted with ethyl acetate (2×30 mL), the combined organic layer was dried over sodium sulfate and evaporated under reduced pressure. The crude residue was purified by silica gel column by eluting with 50% ethyl acetate in pet ether to afford methyl 2-((S)-3-cyclohexyl-2-(4-methoxy-1H-indole-2-carboxamido)propanamido)-3-(2-oxo-8-oxa-1-azaspiro[4.5]decan-3-yl)propanoate (2). TLC system: 10% Methanol in DCM R_{f}: 0.7 LCMS (ESI): m/z 583.64 (M+H)⁺

### N-((2S)-3-Cyclohexyl-1-((1-hydroxy-3-(2-oxo-8-oxa-1-azaspiro[4.5]decan-3-yl)propan-2-yl)amino)-1-oxopropan-2-yl)-4-methoxy-1H-indole-2-carboxamide (3)

To a stirred solution of Methyl 2-((S)-3-cyclohexyl-2-(4-methoxy-1H-indole-2-carboxamido)propanamido)-3-(2-oxo-8-oxa-1-azaspiro[4.5]decan-3-yl)propanoate (2) (1.5 g, 2.57 mmol) in DCM (50 mL) was added 2M LiBH₄ in THF (2.5 mL, 5.14 mmol) at 0° C. and the reaction mixture stirred for 2 h at RT. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford N-((2S)-3-cyclohexyl-1-((1-hydroxy-3-(2-oxo-8-oxa-1-azaspiro[4.5]decan-3-yl)propan-2-yl)amino)-1-oxopropan-2-yl)-4-methoxy-1H-indole-2-carboxamide (3). TLC system: 10% MeOH in DCM R_{f} 0.5 LCMS (ESI): m/z 555.46 (M+H)⁺

### N-((2S)-3-cyclohexyl-1-oxo-1-((1-oxo-3-(2-oxo-8-oxa-1-azaspiro[4.5]decan-3-yl)propan-2-yl)amino)propan-2-yl)-4-methoxy-1H-indole-2-carboxamide (C179)

To a stirred solution of N-((2S)-3-cyclohexyl-1-((1-hydroxy-3-(2-oxo-8-oxa-1-azaspiro[4.5]decan-3-yl)propan-2-yl)amino)-1-oxopropan-2-yl)-4-methoxy-1H-indole-2-carboxamide (3) (150 mg, 0.27 mmol) in ethyl acetate (10 mL) was added Dess-Martin periodinane (229.60 mg, 0.54 mmol) at 0° C. and stirred at RT for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford N-((2S)-3-cyclohexyl-1-oxo-1-((1-oxo-3-(2-oxo-8-oxa-1-azaspiro[4.5]decan-3-yl)propan-2-yl)amino)propan-2-yl)-4-methoxy-1H-indole-2-carboxamide (C179). TLC system: 10% Methanol in DCM R_{f}: 0.4 LCMS (ESI): m/z 553.2 (M+H)⁺

### Example 140: Synthesis of Compounds C201 and C181

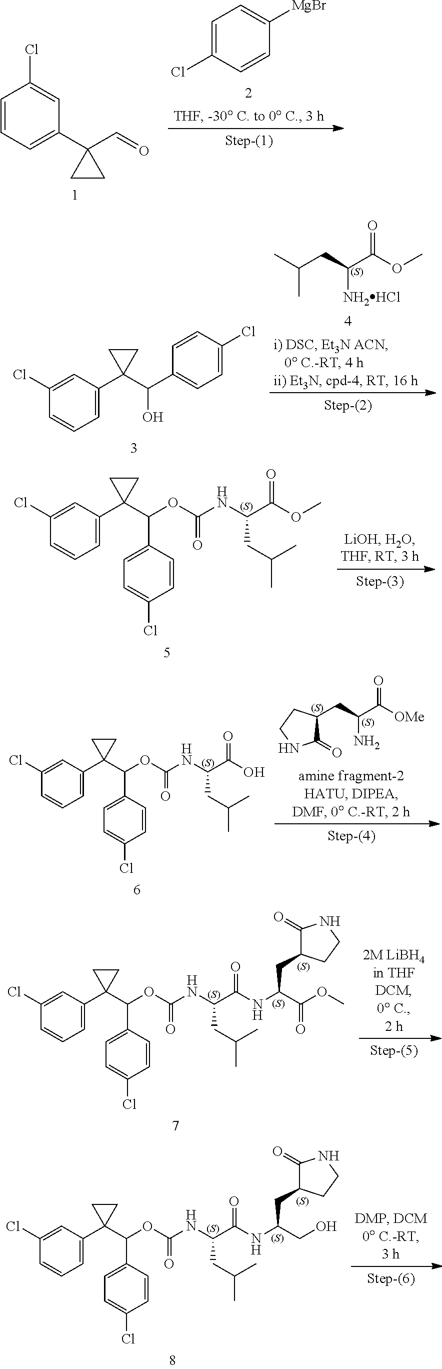

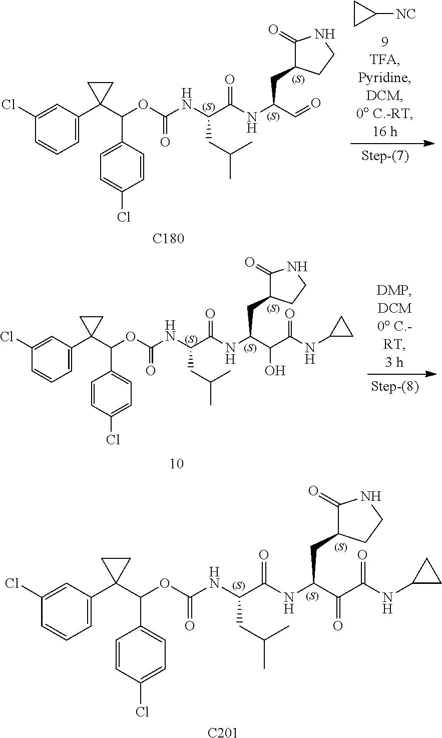

### (4-Chlorophenyl)(1-(3-chlorophenyl) cyclopropyl) methanol (3)

To a stirred solution of (4-chlorophenyl) magnesium bromide (2) (35 mL, 35.55 mmol) THF (70 mL) wad added 1-(3-chlorophenyl) cyclopropane-1-carbaldehyde (1) (3.2 g, 17.77 mmol) at −30° C. and stirred at room temperature for 3 h. Reaction progress was monitored by TLC and LCMS. Reaction mixture was quenched with sat. Ammonium chloride and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 7% ethyl acetate in hexane to afford (4-chlorophenyl) (1-(3-chlorophenyl) cyclopropyl) methanol (3). TLC system: 20% Ethyl acetate in hexane Rf: 0.4 LCMS (ESI): m/z 275.18 [M−OH]⁺

### Methyl (((4-chlorophenyl) (1-(3-chlorophenyl) cyclopropyl) methyl) carbonyl)-leucinate (5)

To a stirred solution of (4-chlorophenyl)(1-(3-chlorophenyl)cyclopropyl)methanol (3) (4 g, 13.69 mmol) in ACN (40 mL) was added N,N′ disuccinimidyl carbonate (8.7 g, 33.98 mmol), followed by triethylamine (6.6 mL, 47.94 mmol) at 0° C. and stirred room temperature for 4 h. The progress of the reaction was monitored by TLC. The reaction mass was used directly in the subsequent reaction.

In another flask, methyl L-leucinate hydrochloride (2) (6.1 g, 34.24 mmol) was taken in ACN (50 mL), and treated with triethylamine (6.6 mL, 47.94 mmol). The resulting reaction mixture was stirred for 5 min, then added above prepared reaction mass drop-wise and the reaction mixture stirred at room temperature for 16 h. Reaction mixture was quenched with ice water (150 mL) and extracted with ethyl acetate (2×50 mL), combined organic layers were washed with brine solution (100 mL), dried over sodium sulfate and evaporated under reduced pressure to afford crude methyl (((4-chlorophenyl) (1-(3-chlorophenyl) cyclopropyl) methoxy) carbonyl)-L-leucinate (5) which was used directly in the next step. TLC system: 20% Ethyl acetate in hexane Rf: 0.3 LCMS (ESI): m/z 486.13 [M+Na]⁺

### (((4-Chlorophenyl) (1-(3-chlorophenyl) cyclopropyl) methoxy) carbonyl)-L-leucine (6)

To a stirred solution of methyl (((4-chlorophenyl)(1-(3-chlorophenyl) cyclopropyl) methoxy) carbonyl)-L-leucinate (5) (5.6 g, 12.09 mmol) in THF (40 mL), water (10 mL) was added lithium hydroxide (870 mg, 36.28 mmol) at 0° C. and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×40 mL), dried over sodium sulfate, concentrated under reduced pressure to afford (((4-chlorophenyl)(1-(3-chlorophenyl)cyclopropyl) methoxy) carbonyl)-L-leucine (6). TLC system: 100% EtOAc Rf: 0.1 LCMS (ESI): m/z 472.19 [M+H]⁺

### Methyl (2S)-2-((2S)-2-((((4-chlorophenyl) (1-(3-chlorophenyl) cyclopropyl) methoxy) carbonyl) amino)-4-methylpentanamido)-3-((S)-2-oxopyrrolidin-3-yl) propanoate (7)

To a stirred solution of (((4-chlorophenyl)(1-(3-chlorophenyl) cyclopropyl) methoxy)carbonyl)-L-leucine (6) (4.5 g, 10.02 mmol) DMF (30 mL) added HATU (9.5 g, 25.05 mmol), DIPEA (4.7 mL, 30.05 mmol) and methyl (S)-2-amino-3-((S)-2-oxopyrrolidin-3-yl)propanoate hydrochloride (amine fragment-2) (2.23 g, 12.02 mmol) at 0° C. simultaneously and stirred at room temperature for 2 h. Reaction mixture was quenched with ice water (100 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 7% methanol in dichloromethane to afford methyl (2S)-2-((2S)-2-((((4-chlorophenyl)(1-(3-chlorophenyl) cyclopropyl)methoxy)carbonyl)amino)-4-methylpentanamido)-3-((S)-2-oxopyrrolidin-3-yl) propanoate (7). TLC system: 10% Methanol in dichloromethane Rf: 0.4 LCMS (ESI): m/z 618.25 [M+H]⁺

### (4-Chlorophenyl)(1-(3-chlorophenyl) cyclopropyl) methyl ((S)-1-(((S)-1-hydroxy-3-((S)-2-oxopyrrolidin-3-yl) propan-2-yl) amino)-4-methyl-1-oxopentan-2-yl) carbamate (8)

To a stirred solution of methyl (2S)-2-((2S)-2-((((4-chlorophenyl)(1-(3-chlorophenyl) cyclopropyl)methoxy)carbonyl)amino)-4-methylpentanamido)-3-((S)-2-oxopyrrolidin-3-yl) propanoate (7) (1.0 g, 1.62 mmol) in dichloromethane (10 mL) was added 2M LiBH₄ in THF (1.2 mL, 2.43 mmol) was added at 0° C. and stirred for 2 h. The progress of the reaction was monitored by TLC and LCMS. Reaction mixture was quenched with saturated NH₄Cl solution and extracted with dichloromethane (2×20 mL), dried over sodium sulfate, concentrated under reduced pressure to afford (4-chlorophenyl)(1-(3-chlorophenyl) cyclopropyl)methyl ((S)-1-(((S)-1-hydroxy-3-((S)-2-oxopyrrolidin-3-yl)propan-2-yl)amino)-4-methyl-1-oxopentan-2-yl) carbamate (8). TLC system: 10% Methanol in dichloromethane Rf: 0.2 LCMS (ESI): m/z 590.34 [M+H]⁺

### (4-Chlorophenyl)(1-(3-chlorophenyl) cyclopropyl) methyl ((S)-4-methyl-1-oxo-1-(((S)-1-oxo-3-((S)-2-oxopyrrolidin-3-yl) propan-2-yl) amino) pentan-2-yl) carbamate (Compound C181)

To a stirred solution of 2-(3-chlorobenzyl)cyclopentyl((S)-1-(((S)-1-hydroxy-3-((S)-2-oxopyrrolidin-3-yl)propan-2-yl) amino)-4-methyl-1-oxopentan-2-yl)carbamate (8) (100 mg, 0.197 mmol) was dissolved in ethyl acetate (10 mL) was added Dess-Martin periodinane (167 mg, 0.394 mmol) at 0° C. and stirred at room temperature for 3 h. Reaction mixture was diluted with dichloromethane (20 mL) and washed with sat. Hypo solution (3×20 mL), sat. NaHCO₃ solution (3×20 mL) and brine (1×20 mL). Organic layer was dried over anhydrous Na₂SO₄, filtered and concentrated to afford crude compound. The crude compound was purified by combi flash NP, eluted 3% methanol in dichloromethane to afford 2-(3-chlorobenzyl)cyclopentyl ((S)-4-methyl-1-oxo-1-(((S)-1-oxo-3-((S)-2-oxopyrrolidin-3-yl)propan-2-yl)amino)pentan-2-yl)carbamate (Compound C181). TLC system: 10% Methanol in dichloromethane Rf: 0.5 LCMS (ESI): m/z 588.1 [M+H]⁺

### (4-Chlorophenyl)(1-(3-chlorophenyl) cyclopropyl) methyl ((2S)-1-(((2S)-4-(cyclopropylamino)-3-hydroxy-4-oxo-1-((S)-2-oxopyrrolidin-3-yl) butan-2-yl) amino)-4-methyl-1-oxopentan-2-yl)carbamate (10)

To a stirred solution of (4-chlorophenyl)(1-(3-chlorophenyl)cyclopropyl)methyl ((S)-4-methyl-1-oxo-1-(((S)-1-oxo-3-((S)-2-oxopyrrolidin-3-yl)propan-2-yl)amino)pentan-2-yl) carbamate (Compound C181) (450 mg, 0.731 mmol) was dissolved in DCM (10 mL) added Pyridine (0.25 mL, 3 vol), isocyanocyclopropane (9) (97 mg, 1.462 mmol) followed by TFA (0.11 mL, 1.462 mmol) at 0° C. and stirred at room temperature for 16 h. The progress of the reaction was monitored by TLC and LCMS. Reaction mixture was diluted with dichloromethane and washed with 1N HCl (2×15 mL) followed by brine (20 mL). Organic layer was dried over anhydrous Na₂SO₄ and evaporated under reduced pressure to afford crude (4-chlorophenyl)(1-(3-chlorophenyl)cyclopropyl)methyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (10). TLC system: 10% Methanol in dichloromethane Rf: 0.4 LCMS (ESI): m/z 673.58 (M+H)⁺

### (4-Chlorophenyl)(1-(3-chlorophenyl) cyclopropyl) methyl ((S)-1-(((S)-4-(cyclopropylamino)-3,4-dioxo-1-((S)-2-oxopyrrolidin-3-yl) butan-2-yl) amino)-4-methyl-1-oxopentan-2-yl) carbamate (Compound C201)

To a stirred solution of (4-chlorophenyl)(1-(3-chlorophenyl)cyclopropyl)methyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (10) (200 mg, 0.297 mmol) in ethyl acetate (10 mL) was added Dess-Martin periodinane (378 mg, 0.892 mmol) at 0° C. and stirred at room temperature for 3 h. The progress of the reaction was monitored by TLC and LCMS. Reaction mixture was filter through celite pad and washed with ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford (4-chlorophenyl)(1-(3-chlorophenyl)cyclopropyl)methyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C201). TLC system: 10% Methanol in dichloromethane Rf: 0.5 LCMS (ESI): m/z 671.1 [M+H]⁺

### Example 141: Synthesis of Compound C195

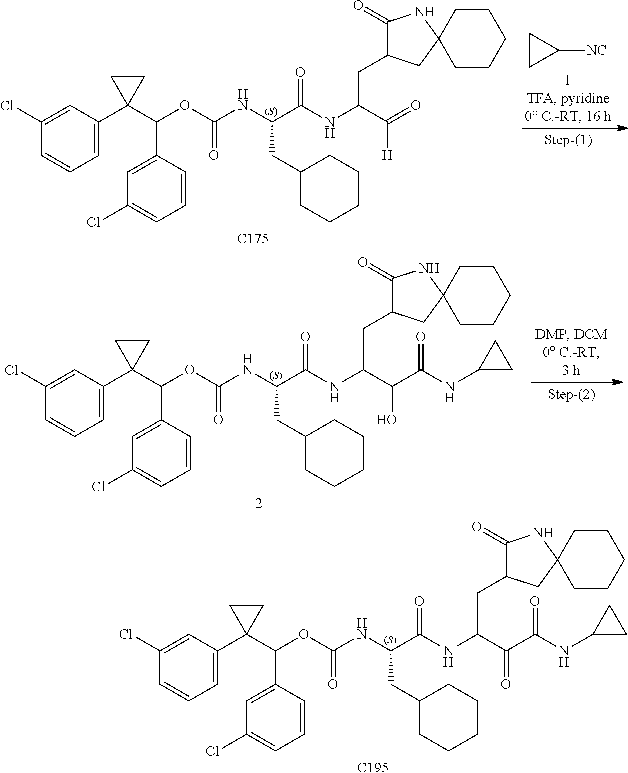

### 2-(3-Chlorobenzyl)cyclo pentyl ((2S)-1-(((2S)-4-(cyclopropylamino)-3-hydroxy-4-oxo-1-((S)-2-oxopyrrolidin-3-yl) butan-2-yl)amino)-4-methyl-1-oxo pentan-2-yl) carbamate (2)

To a stirred solution of 1,2-bis(3-chlorophenyl)ethyl ((2S)-3-cyclohexyl-1-oxo-1-((1-oxo-3-(2-oxo-1-azaspiro[4.5]decan-3-yl)propan-2-yl)amino)propan-2-yl)carbamate (Compound C175) (190 mg, 0.283 mmol) was dissolved in DCM (10 mL) was added Pyridine (0.1 mL, 0.851 mmol), isocyanocyclopropane (1) (0.03 mL, 0.425 mmol) sequentially at 0° C. followed by TFA (0.06 mL, 0.867 mmol) at 0° C. and stirred at room temperature for 16 h. The progress of the reaction was monitored by TLC and LCMS. Reaction mixture was diluted with dichloromethane and washed with 1N HCl (2×15 mL), brine solution (20 mL). The organic layer was dried over anhydrous Na₂SO₄ and evaporated under reduced pressure to afford crude 2-(3-chlorobenzyl)cyclo pentyl ((2S)-1-(((2S)-4-(cyclopropylamino)-3-hydroxy-4-oxo-1-((S)-2-oxopyrrolidin-3-yl) butan-2-yl)amino)-4-methyl-1-oxo pentan-2-yl) carbamate (2). TLC system: 10% Methanol in dichloromethane Rf: 0.5 LCMS (ESI): m/z 754.33 [M+H]⁺

### 1,2-Bis(3-chlorophenyl)ethyl ((2S)-3-cyclohexyl-1-((4-(cyclopropylamino)-3,4-dioxo-1-(2-oxo-1-azaspiro[4.5]decan-3-yl)butan-2-yl)amino)-1-oxopropan-2-yl)carbamate (Compound C195)

To a stirred solution of 1,2-bis(3-chlorophenyl)ethyl ((2S)-3-cyclohexyl-1-((4-(cyclopropylamino)-3-hydroxy-4-oxo-1-(2-oxo-1-azaspiro[4.5]decan-3-yl)butan-2-yl)amino)-1-oxopropan-2-yl)carbamate (2) (175 mg, 0.231 mmol) in DCM (10 mL) was added Dess-Martin periodinane (196 mg, 0.4635 mmol) at 0° C. and stirred at room temperature for 3 h. The progress of the reaction was monitored by TLC and LCMS. Reaction mixture was filter through celite pad and washed with ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford 1,2-bis(3-chlorophenyl)ethyl ((2S)-3-cyclohexyl-1-((4-(cyclopropylamino)-3,4-dioxo-1-(2-oxo-1-azaspiro[4.5]decan-3-yl)butan-2-yl)amino)-1-oxopropan-2-yl)carbamate (Compound C195). TLC system: 10% Methanol in dichloromethane Rf: 0.6 LCMS (ESI): m/z 752.3 (M+H)⁺

### Example 142: Synthesis of Compounds C185 and C184

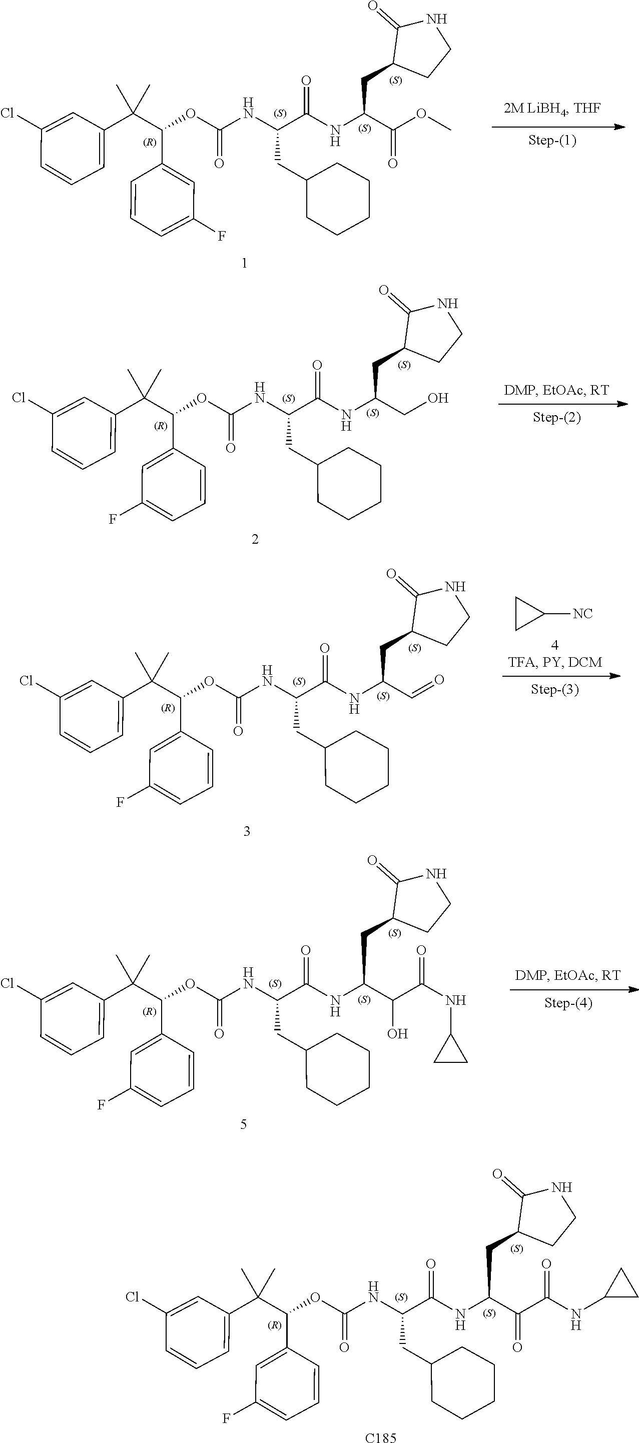

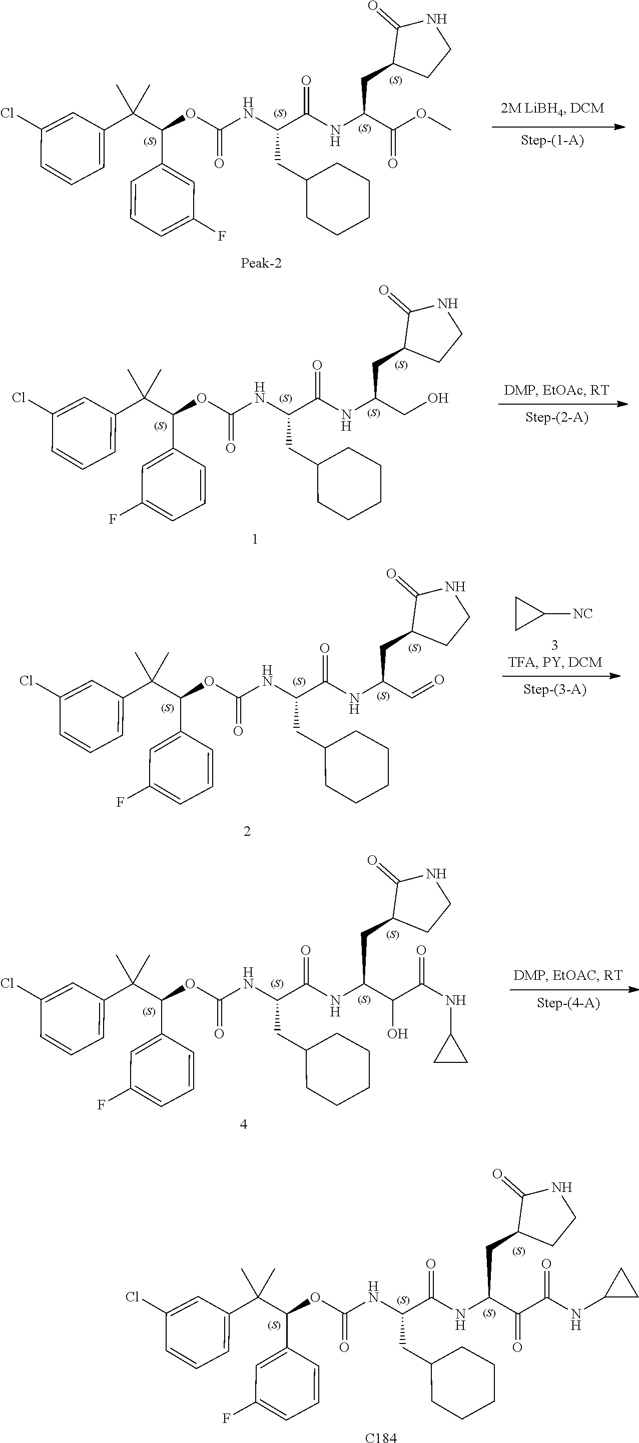

### (R)-2-(3-Chlorophenyl)-1-(3-fluorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (7)

To a stirred solution of methyl (S)-2-((S)-2-((((R)-2-(3-chlorophenyl)-1-(3-fluorophenyl)-2-methylpropoxy)carbonyl)amino)-3-cyclohexylpropanamido)-3-((S)-2-oxopyrrolidin-3-yl) propanoate (1) (0.77 g, 1.19 mmol) in THF (7 mL) was added 2M LiBH₄ in THF (1.19 mL, 2.30 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to afford (R)-2-(3-chlorophenyl)-1-(3-fluoro phenyl)-2-methylpropyl((S)-3-cyclohexyl-1-(((S)-1-hydroxy-3-((S)-2-oxopyrrolidin-3-yl) propan-2-yl)amino)-1-oxopropan-2-yl)carbamate (2). TLC system: Ethyl acetate Rf: 0.2 LCMS (ESI): m/z 616 [M+H]+

### (R)-2-(3-Chlorophenyl)-1-(3-fluorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (3)

To a stirred solution of 2-(3-Chlorobenzyl)cyclopentyl (R)-2-(3-chlorophenyl)-1-(3-fluorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (2) (630 mg, 1.01 mmol) was dissolved in Ethyl acetate (6.5 mL) was added Dess-Martin periodinane (1.31 g, 3.0 mmol) at 0° C. and stirred at room temperature for 3 h. Reaction mixture was diluted with Ethyl acetate (20 mL) and washed with sat. Hypo solution (3×20 mL), sat. NaHCO₃ solution (3×20 mL). Organic layer was dried over anhydrous Na₂SO₄, filtered and concentrated to afford (R)-2-(3-chlorophenyl)-1-(3-fluorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (3). TLC system: 5% Methanol in DCM Rf: 0.3 LCMS (ESI): m/z 614.30 [M+H]+

### (R)-2-(3-chlorophenyl)-1-(3-fluorophenyl)-2-methylpropyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (5)

To a stirred solution of (R)-2-(3-chlorophenyl)-1-(3-fluorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (3) (510 mg, 0.831 mmol) in DCM (10 mL) was added isocyanocyclopropane (4) (111 mg, 1.66 mmol), pyridine (0.262 mL, 3.3 mmol) at 0° C. and the reaction mixture was stirred for 30 minutes at 0° C. and added TFA (0.12 mL, 1.66 mmol) and stirred at room temperature for 16 h. The progress of the reaction was monitored by TLC and LCMS. Reaction mixture was acidified with 1N HCl solution (5 mL) and extracted with DCM (2×10 mL). Organic layer was washed with brine solution (5 mL), dried over Na₂SO₄ and concentrated to get crude compound. The crude compound to afford (R)-2-(3-chlorophenyl)-1-(3-fluorophenyl)-2-methylpropyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (5). TLC system: 5% Methanol in dichloromethane Rf: 0.2 LCMS (ESI): m/z 699.3 [M+H]+

### (R)-2-(3-chlorophenyl)-1-(3-fluorophenyl)-2-methylpropyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C185)

To a stirred solution of (R)-2-(3-chlorophenyl)-1-(3-fluorophenyl)-2-methylpropyl ((2S)-3-cyclohexyl-1-(((2S)-4-(cyclopropylamino)-3-hydroxy-4-oxo-1-((S)-2-oxopyrrolidin-3-yl)butan 2-yl)amino)-1-oxopropan-2-yl)carbamate (5) (500 mg, 0.71 mmol) was dissolved in Ethyl acetate (10 mL) was added Dess-Martin periodinane (750 mg, 1.70 mmol) at 0° C. and stirred at room temperature for 3 h. Reaction mixture was diluted with Ethyl acetate (15 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by normal phase purification afforded (R)-2-(3-chlorophenyl)-1-(3-fluorophenyl)-2-methylpropyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C185). TLC system: 5% Methanol in DCM Rf: 0.3 LCMS (ESI): m/z 697.2 [M+H]+

### (S)-2-(3-Chlorophenyl)-1-(3-fluorophenyl)-2-methylpropyl ((S)-3-cyclohexyl-1-(((S)-1-hydroxy-3-((S)-2-oxopyrrolidin-3-yl)propan-2-yl)amino)-1-oxopropan-2-yl)carbamate (2)

To a stirred solution of methyl (S)-2-((S)-2-((((S)-2-(3-chlorophenyl)-1-(3-fluorophenyl)-2-methylpropoxy)carbonyl)amino)-3-cyclohexylpropanamido)-3-((S)-2-oxopyrrolidin-3-yl) propanoate (1) (0.700 g, 1.08 mmol) in DCM (7 mL) was added 2M LiBH₄ in THF (1 mL, 2.177 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to afford (S)-2-(3-chlorophenyl)-1-(3-fluoro phenyl)-2-methylpropyl((S)-3-cyclohexyl-1-(((S)-1-hydroxy-3-((S)-2-oxopyrrolidin-3-yl) propan-2-yl)amino)-1-oxopropan-2-yl)carbamate (2). TLC system: Ethyl acetate Rf: 0.2 LCMS (ESI): m/z 616.61 [M+H]+

### (S)-2-(3-Chlorophenyl)-1-(3-fluorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (3)

To a stirred solution of 2-(3-Chlorobenzyl)cyclopentyl (S)-2-(3-chlorophenyl)-1-(3-fluorophenyl)-2-methylpropyl ((S)-3-cyclohexyl-1-(((S)-1-hydroxy-3-((S)-2-oxopyrrolidin-3-yl) propan-2-yl)amino)-1-oxopropan-2-yl)carbamate (2) (620 mg, 1.01 mmol) was dissolved in Ethyl acetate (6.5 mL) was added Dess-Martin periodinane (1.282 g, 3.0 mmol) at 0° C. and stirred at room temperature for 3 h. Reaction mixture was diluted with Ethyl acetate (20 mL) and washed with sat. Hypo solution (3×20 mL), sat. NaHCO₃ solution (3×20 mL). Organic layer was dried over anhydrous Na₂SO₄, filtered and concentrated to afford (S)-2-(3-chlorophenyl)-1-(3-fluorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl)carbamate (3). TLC system: 10% Methanol in DCM Rf: 0.4 LCMS (ESI): m/z 614.30 [M+H]+

### (S)-2-(3-chlorophenyl)-1-(3-fluorophenyl)-2-methylpropyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (10)

To a stirred solution of (S)-2-(3-chlorophenyl)-1-(3-fluorophenyl)-2-methylpropyl ((S)-3-cyclohexyl-1-oxo-1-(((S)-1-oxo-3-((S)-2-oxopyrrolidin-3-yl)propan-2-yl)amino)propan-2-yl) carbamate (3) (600 mg, 0.978 mmol) in DCM (10 mL) was added isocyanocyclopropane (4) (131.1 mg, 1.957 mmol), pyridine (0.3 mL, 3.915 mmol) at 0° C. and the reaction mixture was stirred for 30 minutes at 0° C. and added TFA (0.15 mL, 1.95 mmol) and stirred at room temperature for 16 h. The progress of the reaction was monitored by TLC and LCMS. Reaction mixture was acidified with 1N HCl solution (5 mL) and extracted with DCM (2×10 mL). Organic layer was washed with brine solution (5 mL), dried over Na₂SO₄ and concentrated to get crude compound. The crude compound to afford (S)-2-(3-chlorophenyl)-1-(3-fluorophenyl)-2-methylpropyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (5). TLC system: 10% Methanol in dichloromethane Rf: 0.2 LCMS (ESI): m/z 699.2 [M+H]+

### (S)-2-(3-chlorophenyl)-1-(3-fluorophenyl)-2-methylpropyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C184)

To a stirred solution of (S)-2-(3-chlorophenyl)-1-(3-fluorophenyl)-2-methylpropyl ((2S)-3-cyclohexyl-1-(((2S)-4-(cyclopropylamino)-3-hydroxy-4-oxo-1-((S)-2-oxopyrrolidin-3-yl)butan 2-yl)amino)-1-oxopropan-2-yl)carbamate (5) (400 mg, 0.71 mmol) was dissolved in Ethyl acetate (10 mL) was added Dess-Martin periodinane (727 mg, 1.716 mmol) at 0° C. and stirred at room temperature for 3 h. Reaction mixture was diluted with Ethyl acetate (15 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by normal phase purification afforded (S)-2-(3-chlorophenyl)-1-(3-fluorophenyl)-2-methylpropyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C184). TLC system: 10% Methanol in DCM Rf: 0.3 LCMS (ESI): m/z 697.2 [M+H]+

### Example 143: Synthesis of Compounds C187 and C203

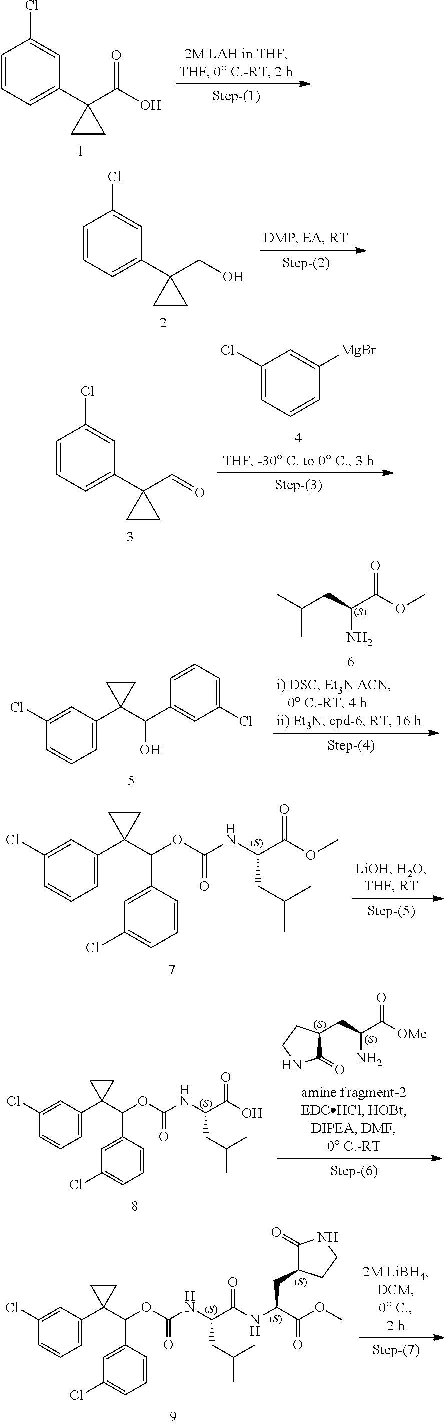

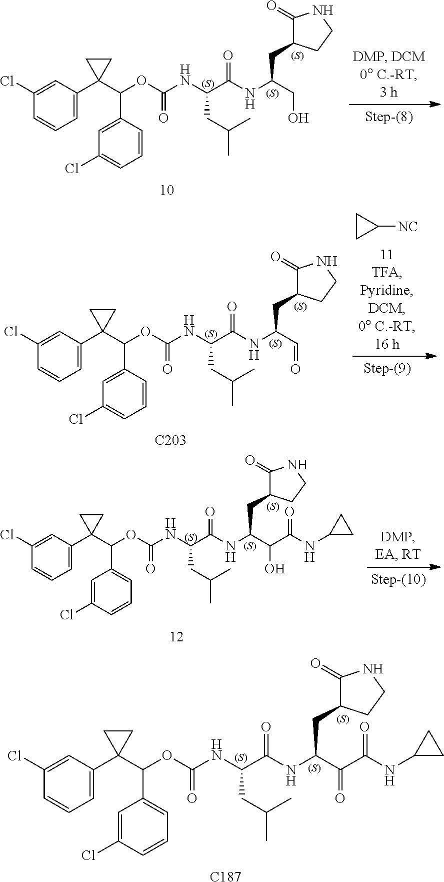

### (1-(3-chlorophenyl)cyclopropyl)methanol (2)

To a stirred solution of 1-(3-chlorophenyl)cyclopropane-1-carboxylic acid (1) (10 g, 51.02 mmol) in THF (200 mL) ware added LiAlH₄ drop wise at 0° C. and stirred at room temperature for 2 h. The progress of the reaction was monitored by TLC and LCMS. Reaction mixture quenched with saturated NH₄Cl solution and extracted with ethyl acetate (2×100 mL). Combined organic layer and washed with water (2×50 mL), dried over sodium sulfate, concentrated under reduced pressure. The crude residue was purified by combi-flash compound eluted at 15% Ethyl acetate in pet ether to afford compound (1-(3-chlorophenyl)cyclopropyl)methanol (2). TLC system: 30% Ethyl acetate in hexane Rf: 0.6

### 1-(3-chlorophenyl)cyclopropane-1-carbaldehyde (3)

To a stirred solution of (1-(3-chlorophenyl)cyclopropyl)methanol (2) (8 g, 4.39 mmol) was dissolved in ethyl acetate (80 mL) was added Dess-Martin periodinane (2.79 g, 6.59 mmol) at 0° C. and stirred at room temperature for 3 h. Reaction mixture was diluted with Ethyl acetate (100 mL) and washed with sat. Hypo solution (3×50 mL), sat. NaHCO₃ solution (3×50 mL). Organic layer was dried over anhydrous Na₂SO₄, filtered and concentrated to afford 1-(3-chlorophenyl)cyclopropane-1-carbaldehyde (3) vTLC system: 10% Ethyl acetate in hexane Rf: 0.7 LCMS (ESI): m/z 181.1 [M+H]⁻

### (3-chlorophenyl)(1-(3-chlorophenyl)cyclopropyl)methanol (5)

To a stirred solution of 1-(3-chlorophenyl)cyclopropane-1-carbaldehyde (3) (6.5 g, 36.11 mmol) in THF (200 mL) ware added (3-chlorophenyl)magnesium bromide (4) drop wise at −30° C. and stirred at room temperature for 3 h. The progress of the reaction was monitored by TLC and LCMS. Reaction mixture was quenched with saturated ammonium chloride solution and extracted with ethyl acetate (2×50 mL). Combined organic layer and washed with water (2×50 mL), dried over sodium sulfate, concentrated under reduced pressure. The crude residue was purified by combi-flash compound eluted at 08% Ethyl acetate in pet ether to afford (3-chlorophenyl)(1-(3-chlorophenyl)cyclopropyl)methanol (5) TLC system: 10% Ethyl acetate in hexane Rf: 0.7 LCMS (ESI): m/z 315.13 [M+Na+H]⁺

### Methyl (((3-chlorophenyl)(1-(3-chlorophenyl)cyclopropyl)methoxy)carbonyl)-L-leucinate (7)

To a stirred solution of (3-chlorophenyl)(1-(3-chlorophenyl)cyclopropyl)methanol (5) (5.0 g, 17.06 mmol) in Acetonitrile (50 mL) ware added TEA (8.30 mL, 59.7 mmol), DSC (10.9 g, 42.01 mmol) at 0° C. and stirred at room temperature for 3 h The progress of the reaction was monitored by TLC Then added methyl (S)-2-amino-3-cyclohexylpropanoate (6) and stirred at room temperature for 16 h. The progress of the reaction was monitored by TLC and LCMS. Reaction mixture was diluted with ethyl acetate (50 mL) and extracted with water (50 mL), organic layer was dried over sodium sulfate, filtered and evaporated under reduced pressure. The crude residue was purified by combi-flash, compound eluted at 10% ethyl acetate in pet ether to afford methyl (((3-chlorophenyl)(1-(3-chlorophenyl)cyclopropyl)methoxy)carbonyl)-L-leucinate (7). TLC system: 10% Ethyl acetate in hexane Rf: 0.4 LCMS (ESI): m/z 464.16 [M+H]⁺

### (((3-chlorophenyl)(1-(3-chlorophenyl)cyclopropyl)methoxy)carbonyl)-L-leucine (8)

To a stirred solution of methyl methyl (((3-chlorophenyl)(1-(3-chlorophenyl)cyclopropyl)methoxy)carbonyl)-L-leucinate (7) (4.9 g, 10.50 mmol) in THF (30 mL), water (20 mL) was added lithium hydroxide (1.33 g, 31.66 mmol) at 0° C. and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (((3-chlorophenyl)(1-(3-chlorophenyl)cyclopropyl)methoxy)carbonyl)-L-leucine (8). TLC system: 30% Ethyl acetate in hexane Rf: 0.2 LCMS (ESI): m/z 450.1 [M+H]⁺

### Methyl (2S)-2-((2S)-2-((((3-chlorophenyl)(1-(3-chlorophenyl)cyclopropyl)methoxy)carbonyl)amino)-4-methylpentanamido)-3-((S)-2-oxopyrrolidin-3-yl)propanoate (9)

To a stirred solution of (((3-chlorophenyl)(1-(3-chlorophenyl)cyclopropyl)methoxy)carbonyl)-L-leucine (8) (1.2 g, 2.60 mmol) DMF (12 mL) added EDC.HCl (765 mg, 4.01 mmol), HOBt (541 mg, 4.01 mmol), DIPEA (1.42 mL, 8.01 mmol) and methyl (S)-2-amino-3-((S)-2-oxopyrrolidin-3-yl)propanoate (amine fragment-2) (1.08 g, 5.847 mmol) at 0° C. simultaneously and stirred at room temperature for 16 h. The progress of the reaction was monitored by TLC and LCMS. After 16 h, the reaction mixture was diluted with ice water (20 mL), extracted with ethyl acetate (2×30 mL). The combined organic layer was dried over sodium sulfate, filtered and evaporated under reduced pressure. The crude residue was purified by combi-flash compound eluted at 50% Ethyl acetate in pet ether to afford methyl(2S)-2-((2S)-2-((((3-chlorophenyl)(1-(3-chlorophenyl)cyclopropyl)methoxy)carbonyl)-amino)-4-methylpentanamido)-3-((S)-2-oxopyrrolidin-3-yl)propanoate (9) (1.2 g, 1.96 mmol, 75% yield). TLC system: Ethyl acetate Rf: 0.5 LCMS (ESI): m/z 618.5 [M+H]⁺

### (3-Chlorophenyl)(1-(3-chlorophenyl)cyclopropyl)methyl ((S)-1-(((S)-1-hydroxy-3-((S)-2-oxopyrrolidin-3-yl)propan-2-yl)amino)-4-methyl-1-oxopentan-2-yl)carbamate (10)

To a stirred solution of methyl(2S)-2-((2S)-2-((((3-chlorophenyl)(1-(3-chlorophenyl)cyclopropyl)methoxy)carbonyl)amino)-4-methylpentanamido)-3-((S)-2-oxopyrrolidin-3-yl)propanoate (9) (0.750 g, 1.20 mmol) in THF (10 mL) was added 2M LiBH4 in THF (1.55 mL, 2.45 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to (3-chlorophenyl)(1-(3-chlorophenyl)cyclopropyl)methyl ((S)-1-(((S)-1-hydroxy-3-((S)-2-oxopyrrolidin-3-yl)propan-2-yl)amino)-4-methyl-1-oxopentan-2-yl)carbamate (10). TLC system: Ethyl acetate Rf: 0.2 LCMS (ESI): m/z 590.5 (M+H)⁺

### (3-chlorophenyl)(1-(3-chlorophenyl)cyclopropyl)methyl ((S)-4-methyl-1-oxo-1-(((S)-1-oxo-3-((S)-2-oxopyrrolidin-3-yl)propan-2-yl)amino)pentan-2-yl)carbamate (Compound C203)

To a stirred solution of ((S)-1-(((S)-1-hydroxy-3-((S)-2-oxopyrrolidin-3-yl)propan-2-yl)amino)-4-methyl-1-oxopentan-2-yl)carbamate (10) (320 mg, 0.54 mmol) was dissolved in Ethyl acetate (5 mL) was added Dess-Martin periodinane (689 mg, 1.62 mmol) at 0° C. and stirred at room temperature for 3 h. Reaction mixture was diluted with Ethyl acetate (20 mL) and washed with sat. Hypo solution (3×20 mL), sat. NaHCO₃ solution (3×20 mL). Organic layer was dried over anhydrous Na₂SO₄, filtered and evaporated under reduced pressure. The crude residue was purified by combi-flash compound eluted at 5% Dichloromethane in Methanol to afford (3-chlorophenyl)(1-(3-chlorophenyl)cyclopropyl)methyl ((S)-4-methyl-1-oxo-1-(((S)-1-oxo-3-((S)-2-oxopyrrolidin-3-yl)propan-2-yl)amino)pentan-2-yl)carbamate (Compound C203). TLC system: Ethyl acetate Rf: 0.3 LCMS (ESI): m/z 588.0 (M+H)⁺

### (3-Chlorophenyl)(1-(3-chlorophenyl)cyclopropyl)methyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (12)

To a stirred solution of ((S)-4-methyl-1-oxo-1-(((S)-1-oxo-3-((S)-2-oxopyrrolidin-3-yl)propan-2-yl)amino)pentan-2-yl)carbamate (Compound C203) (290 mg, 0.49 mmol) in DCM (10 mL) was added isocyanocyclopropane (11) (66 mg, 0.98 mmol), pyridine (0.15 mL, 1.97 mmol) at 0° C. and the reaction mixture was stirred for 30 minutes at 0° C. and added TFA (0.075 mL, 0.91 mmol) and stirred at room temperature for 16 h. The progress of the reaction was monitored by TLC and LCMS. Reaction mixture was acidified with 1N HCl solution (5 mL) and extracted with DCM (2×10 mL). Organic layer was washed with brine solution (5 mL), dried over Na₂SO₄ and concentrated to get crude compound. The crude compound to afford (3-chlorophenyl)(1-(3-chlorophenyl)cyclopropyl)methyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (12). TLC system: 5% Methanol in dichloromethane Rf: 0.2 LCMS (ESI): m/z 673.2 [M+H]⁺

### (3-chlorophenyl)(1-(3-chlorophenyl)cyclopropyl)methyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C187)

To a stirred solution of ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (12) (290 mg, 0.43 mmol) was dissolved in Ethyl acetate (10 mL) was added Dess-Martin periodinane (457 mg, 1.02 mmol) at 0° C. and stirred at room temperature for 3 h. Reaction mixture was diluted with Ethyl acetate (15 mL) and washed with sat. Hypo solution (3×20 mL), sat. NaHCO₃ solution (3×20 mL). Organic layer was dried over anhydrous Na2SO4, filtered and concentrated to afford crude, this crude was purified by normal phase purification afforded (3-chlorophenyl)(1-(3-chlorophenyl)cyclopropyl)methyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C187). TLC system: 5% Methanol in DCM Rf: 0.3 LCMS (ESI): m/z 671.1 (M+H)⁺

### Example 144: Synthesis of Compounds C188 and C212

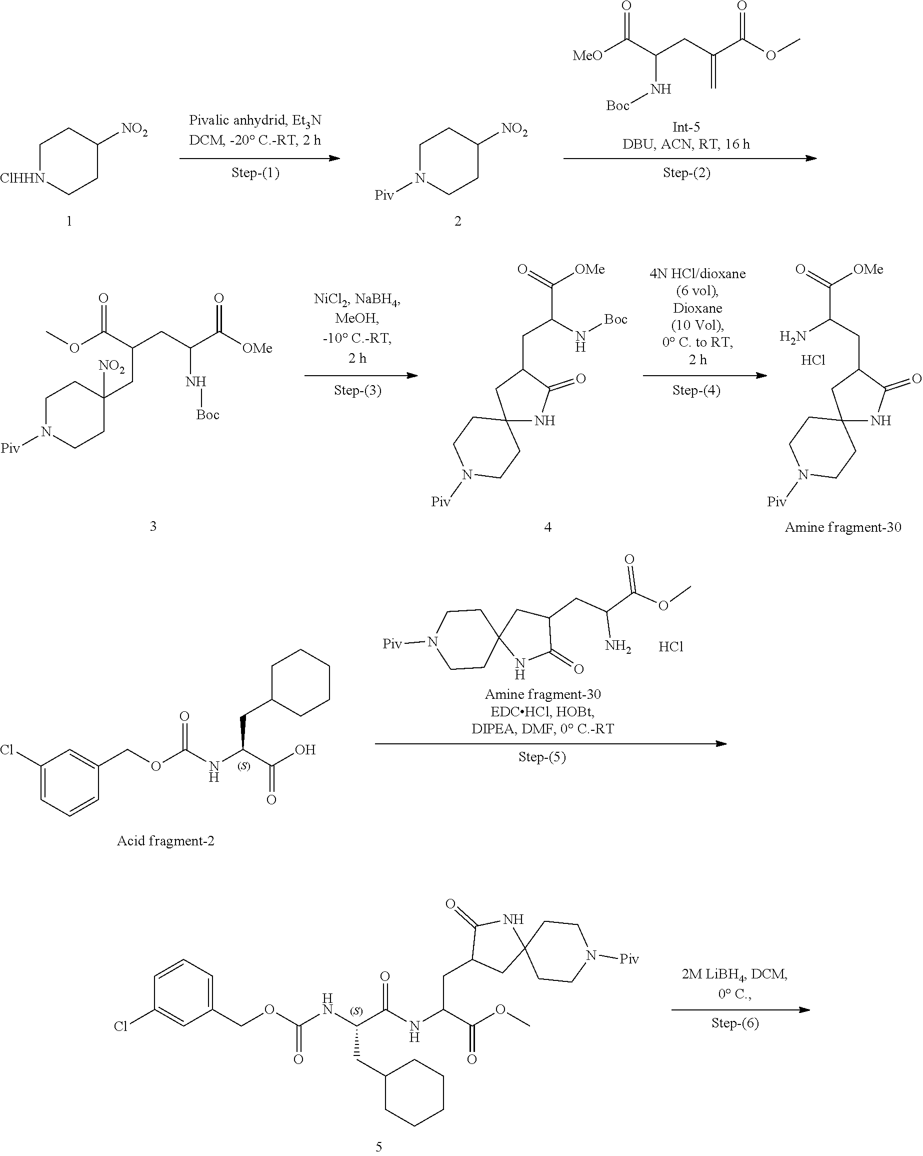

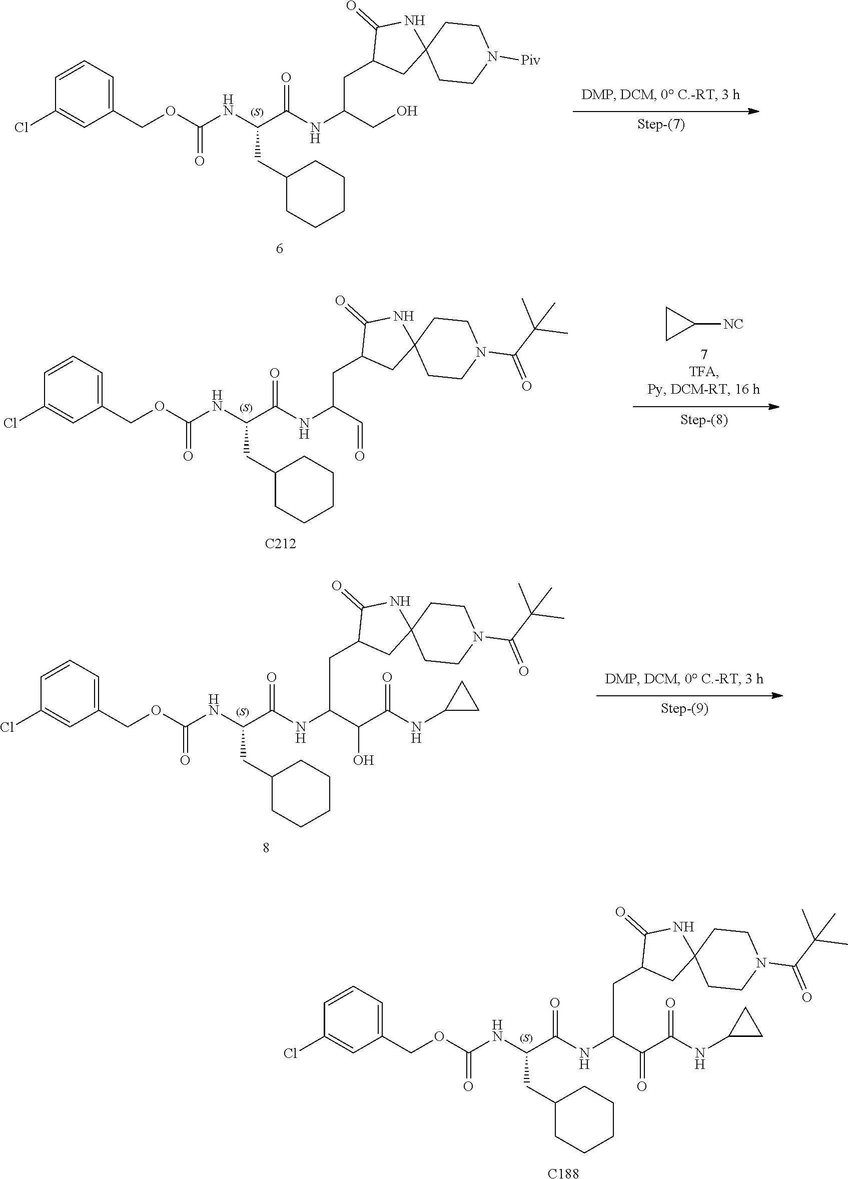

### 2,2-Dimethyl-1-(4-nitropiperidin-1-yl)propan-1-one (2)

To a stirred solution 4-nitropiperidine hydrochloride (1) (2.9 g, 22.307 mmol) was dissolved in DCM (30 mL) was added Pivalic anhydride (6.5 mL, 33.461 mmol) and Et₃N (9.4 mL, 66.921 mmol) at −20° C. simultaneously and stirred for 2 h. The progress of the reaction was monitored by TLC and LCMS. Reaction mixture was quenched with ice water (40 mL) extracted with DCM (2×30 mL), organic layers were washed with water (2×20 ml), brine solution (20 mL), dried over sodium sulfate and evaporated under reduced pressure. The crude residue was purified by normal phase chromatography to afford 2,2-dimethyl-1-(4-nitropiperidin-1-yl)propan-1-one (2) (2.5 g, 11.68 mmol, 52% yield) as an colorless liquid. TLC system: 30% EtOAc in pet ether R_{f}: 0.3 LCMS (ESI): m/z 215.18 [M+H]⁺

### Dimethyl (2S)-2-((tert-butoxycarbonyl)amino)-4-((4-nitro-1-pivaloylpiperidin-4-yl)methyl)pentanedioate (3)

To a stirred solution of dimethyl (S)-2-((tert-butoxycarbonyl)amino)-4-methylenepentanedioate (Intermediate-5) (3.84 g, 13.395 mmol) in ACN (30 mL) was added 2,2-dimethyl-1-(4-nitropiperidin-1-yl)propan-1-one (2) (2.4 g, 11.162 mmol) and DBU (2.19 mL, 14.42 mmol) at 0° C. and stirred at room temperature for 16 h. The progress of the reaction was monitored by TLC and LCMS. Reaction mixture was evaporated under reduced pressure. The crude residue was purified by normal phase chromatography to afford dimethyl (2S)-2-((tert-butoxycarbonyl)amino)-4-((4-nitro-1-pivaloylpiperidin-4-yl)methyl)pentanedioate (3). TLC system: 30% EtOAc in Pet ether R_{f}: 0.4 LCMS (ESI): m/z 502.47 (M+H)⁺

### Methyl (2S)-2-((tert-butoxycarbonyl)amino)-3-(2-oxo-8-pivaloyl-1,8-diazaspiro[4.5]decan-3-yl)propanoate (4)

To a stirred solution of dimethyl (2S)-2-((tert-butoxycarbonyl)amino)-4-((4-nitro-1-pivaloylpiperidin-4-yl)methyl)pentanedioate (3) (4.9 g, 9.760 mmol) in methanol (10 mL) was added nickel chloride (1.38 g, 10.737 mmol), followed by sodium borohydride (1.85 g, 48.80 mmol) at −10° C. and stirred at room temperature for 2 h. The progress of the reaction was monitored by TLC and LCMS. The reaction mixture was quenched with sat. ammonium chloride (50 mL) and extracted with 10% MeOH IN DCM (3×25 mL), combined organic layers were washed with water (2×20 ml), brine solution (25 mL), dried over sodium sulfate and evaporated under reduced pressure. The crude residue was purified by normal phase chromatography to afford methyl (2S)-2-((tert-butoxycarbonyl)amino)-3-(2-oxo-8-pivaloyl-1,8-diazaspiro[4.5]decan-3-yl)propanoate (4). TLC system: 10% MeOH/DCM R_{f}: 0.2 LCMS (ESI): m/z 440.45 (M+H)⁺

### Methyl (2S)-2-amino-3-(2-oxo-8-pivaloyl-1,8-diazaspiro[4.5]decan-3-yl)propanoate hydrochloride (Amine fragment-30)

To a stirred solution of methyl (2S)-2-((tert-butoxycarbonyl)amino)-3-(2-oxo-8-pivaloyl-1,8-diazaspiro[4.5]decan-3-yl)propanoate (4) (3.2 g, 7.954 mmol) in DCM (10 mL) was added 1,4-dioxane. HCl (20 mL) at 0° C. and stirred at room temperature for 2 h. The progress of the reaction was monitored by TLC. Reaction mixture was evaporated under reduced pressure. The crude residue was trituration with n-pentane to afford methyl (2S)-2-amino-3-(2-oxo-8-pivaloyl-1,8-diazaspiro[4.5]decan-3-yl)propanoate hydrochloride (Amine fragment 30). TLC system: 10% MeOH/DCM R_{f}: 0.1 LCMS (ESI): m/z 340.35 (M+H)⁺

### Methyl 2-((S)-2-((((3-chlorobenzyl)oxy)carbonyl)amino)-3-cyclohexylpropanamido)-3-(2-oxo-8-pivaloyl-1,8-diazaspiro[4.5]decan-3-yl)propanoate (5)

To a stirred solution of (S)-2-((((3-chlorobenzyl)oxy)carbonyl)amino)-3-cyclohexylpropanoic acid (Acid fragment-2) (1.0 g, 2.941 mmol) in DMF (10 mL), was added EDC.HCl (1.0 g, 4.411 mmol), HOBt (0.842 g, 4.411 mmol), DIPEA (1.52 mL, 8.823 mmol) and methyl (2S)-2-amino-3-(2-oxo-8-pivaloyl-1,8-diazaspiro[4.5]decan-3-yl)propanoate hydrochloride (Amine fragment-30) (1.55 g, 4.218 mmol) at 0° C. subsequently and stirred at room temperature for 16 h. Reaction mixture was diluted with ice water (20 mL), extracted with ethyl acetate (2×60 mL), dried over sodium sulfate and evaporated under reduced pressure. The crude residue was purified by combi-flash NP by eluting at 5% methanol in dichloromethane to afforded methyl (2S)-2-((S)-2-((((3-chlorobenzyl)oxy)carbonyl)amino)-3-cyclohexylpropanamido)-3-(2-oxo-8-pivaloyl-1,8-diazaspiro[4.5]decan-3-yl)propanoate (5). TLC system: 5% Methanol in DCM Rf: 0.5 LCMS (ESI): m/z 661.61 [M+H]⁺

### 3-Chlorobenzyl ((2S)-3-cyclohexyl-1-((1-hydroxy-3-(2-oxo-8-pivaloyl-1,8-diazaspiro[4.5]decan-3-yl)propan-2-yl)amino)-1-oxopropan-2-yl)carbamate (6)

To a stirred solution methyl (2S)-2-((S)-2-((((3-chlorobenzyl)oxy)carbonyl)amino)-3-cyclohexylpropanamido)-3-(2-oxo-8-pivaloyl-1,8-diazaspiro[4.5]decan-3-yl)propanoate (5) (1.0 g, 1.371 mmol) in THF (10 mL) was added 2M LiBH₄ in THF (1.3 mL, 2.743 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to afforded 3-chlorobenzyl ((2S)-3-cyclohexyl-1-((1-hydroxy-3-(2-oxo-8-pivaloyl-1,8-diazaspiro[4.5]decan-3-yl)propan-2-yl)amino)-1-oxopropan-2-yl)carbamate (6). TLC system: 10% Methanol in DCM Rf: 0.2 LCMS (ESI): m/z 633.4 (M+H)⁺

### 3-Chlorobenzyl ((2S)-3-cyclohexyl-1-oxo-1-((1-oxo-3-(2-oxo-8-pivaloyl-1,8-diazaspiro[4.5]decan-3-yl)propan-2-yl)amino)propan-2-yl)carbamate (Compound C212)

To a stirred solution of 3-chlorobenzyl ((2S)-3-cyclohexyl-1-((1-hydroxy-3-(2-oxo-8-pivaloyl-1,8-diazaspiro[4.5]decan-3-yl)propan-2-yl)amino)-1-oxopropan-2-yl)carbamate (6) (200 mg, 0.31 mmol) was dissolved in EtOAc (10 mL) was added Dess-Martin periodinane (534 mg, 1.26 mmol) at 0° C. and stirred at room temperature for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by normal phase purification afforded 3-chlorobenzyl ((2S)-3-cyclohexyl-1-oxo-1-((1-oxo-3-(2-oxo-8-pivaloyl-1,8-diazaspiro[4.5]decan-3-yl)propan-2-yl)amino)propan-2-yl)carbamate (Compound C212). TLC system: 5% Methanol in DCM Rf: 0.3 LCMS (ESI): m/z 631.3 (M+H)⁺

### 3-Chlorobenzyl ((2S)-3-cyclohexyl-1-((4-(cyclopropylamino)-3-hydroxy-4-oxo-1-(2-oxo-8-pivaloyl-1,8-diazaspiro[4.5]decan-3-yl)butan-2-yl)amino)-1-oxopropan-2-yl)carbamate (8)

To a stirred solution of 3-chlorobenzyl ((2S)-3-cyclohexyl-1-oxo-1-((1-oxo-3-(2-oxo-8-pivaloyl-1,8-diazaspiro[4.5]decan-3-yl)propan-2-yl)amino)propan-2-yl)carbamate (Compound C212) (300 mg, 0.47 mmol) in DCM (10 mL) was added isocyanocyclopropane (7) (48 mg, 0.71 mol), pyridine (0.15 mL, 1.716 mmol) at 0° C. and the reaction mixture was stirred for 30 minutes at 0° C. and added TFA (0.1 mL, 0.858 mmol) and stirred at room temperature for 16 h. The progress of the reaction was monitored by TLC and LCMS. Reaction mixture was acidified with 1N HCl solution (5 mL) and extracted with DCM (2×10 mL). Organic layer was washed with brine solution (5 mL), dried over Na₂SO₄ and concentrated to get crude compound. The crude compound to afford 3-chlorobenzyl ((2S)-3-cyclohexyl-1-((4-(cyclopropylamino)-3-hydroxy-4-oxo-1-(2-oxo-8-pivaloyl-1,8-diazaspiro[4.5]decan-3-yl)butan-2-yl)amino)-1-oxopropan-2-yl)carbamate (8). TLC system: 5% Methanol in dichloromethane Rf: 0.2 LCMS (ESI): m/z 716.6 [M+H]⁺

### 3-Chlorobenzyl ((2S)-3-cyclohexyl-1-((4-(cyclopropylamino)-3,4-dioxo-1-(2-oxo-8-pivaloyl-1,8-diazaspiro[4.5]decan-3-yl)butan-2-yl)amino)-1-oxopropan-2-yl)carbamate (Compound C188)

To a stirred solution of 3-chlorobenzyl ((2S)-3-cyclohexyl-1-((4-(cyclopropylamino)-3-hydroxy-4-oxo-1-(2-oxo-8-pivaloyl-1,8-diazaspiro[4.5]decan-3-yl)butan-2-yl)amino)-1-oxopropan-2-yl)carbamate (8) (260 mg, 0.36 mmol) was dissolved in EtoAc (10 mL) was added Dess-Martin periodinane (462 mg, 1.019 mmol) at 0° C. and stirred at room temperature for 3 h. Reaction mixture was diluted with dichloromethane (10 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by normal phase purification 3-chlorobenzyl ((2S)-3-cyclohexyl-1-((4-(cyclopropylamino)-3,4-dioxo-1-(2-oxo-8-pivaloyl-1,8-diazaspiro[4.5]decan-3-yl)butan-2-yl)amino)-1-oxopropan-2-yl)carbamate (Compound C188). TLC system: 5% Methanol in DCM Rf: 0.3 LCMS (ESI): m/z 714.2 (M+H)⁺

### Example 145: Synthesis of Compounds C193 and C190

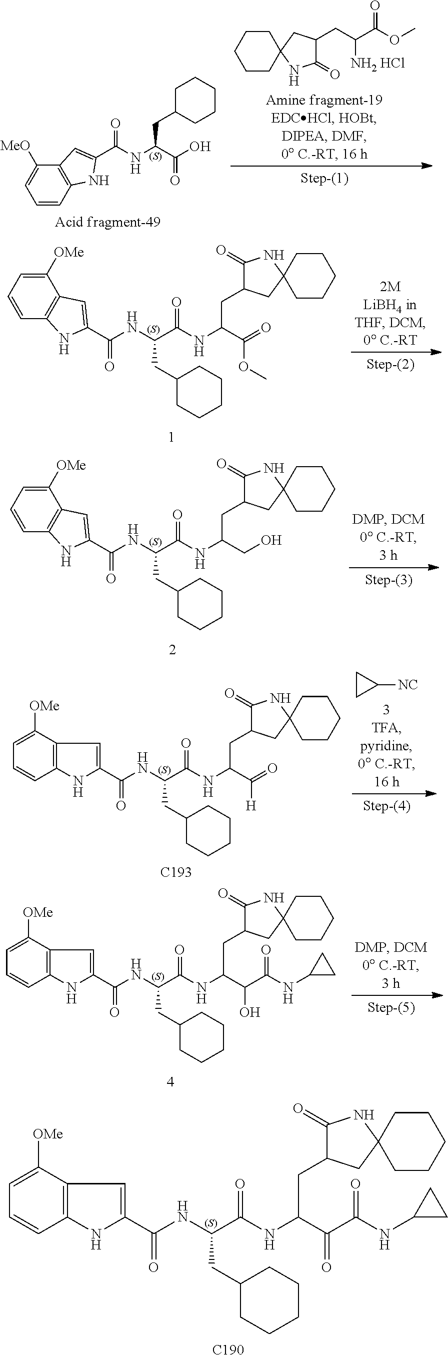

### Methyl 2-((S)-3-cyclohexyl-2-(4-methoxy-1H-indole-2-carboxamido)propanamido)-3-(2-oxo-1-azaspiro[4.5]decan-3-yl)propanoate (1)

To a stirred solution of (S)-3-cyclohexyl-2-(4-methoxy-1H-indole-2-carboxamido)propanoic acid (acid fragment-49) (1 g, 2.90 mmol) in DMF (10 mL) was added EDC.HCl (833 mg, 4.36 mmol), HOBt (590 mg, 4.36 mmol), DIPEA (1.6 mL, 8.7 mmol) and methyl 2-amino-3-(2-oxo-1-azaspiro[4.5]decan-3-yl)propanoate hydrochloride (amine fragment-19) (843 mg, 2.90 mmol) at 0° C. simultaneously and stirred at room temperature for 16 h. Reaction mixture was quenched with ice water (80 mL) and extracted with ethyl acetate (2×50 mL). The organic layer was washed with brine solution (2×50 mL), dried over sodium sulfate and evaporated under reduced pressure. The crude residue was purified by combi-flash NP, compound eluted at 5% methanol in dichloromethane to afford methyl 2-((S)-3-cyclohexyl-2-(4-methoxy-1H-indole-2-carboxamido)propanamido)-3-(2-oxo-1-azaspiro[4.5]decan-3-yl)propanoate (1). TLC system: 10% Methanol in dichloromethane Rf: 0.4 LCMS (ESI): m/z 580.3 [M+H]⁺

### N-((2S)-3-cyclohexyl-1-((1-hydroxy-3-(2-oxo-1-azaspiro[4.5]decan-3-yl)propan-2-yl)amino)-1-oxopropan-2-yl)-4-methoxy-1H-indole-2-carboxamide (2)

To a stirred solution of methyl 2-((S)-3-cyclohexyl-2-(4-methoxy-1H-indole-2-carboxamido)propanamido)-3-(2-oxo-1-azaspiro[4.5]decan-3-yl)propanoate (1) (900 mg, 1.55 mmol) in DCM (9 mL) was added 2M LiBH₄ in THF (1.5 mL, 3.10 mmol) at 0° C. and the reaction mixture stirred for 2 h at 0° C. The progress of the reaction was monitored by TLC and LCMS. Reaction mixture was quenched with sat. ammonium chloride solution (30 mL) and extracted with DCM (2×20 mL). Organic layer was washed with brine solution (30 mL), dried over Na₂SO₄ and concentrated to afford N-((2S)-3-cyclohexyl-1-((1-hydroxy-3-(2-oxo-1-azaspiro[4.5]decan-3-yl)propan-2-yl)amino)-1-oxopropan-2-yl)-4-methoxy-1H-indole-2-carboxamide (2). TLC system: 10% Methanol in dichloromethane Rf: 0.2 LCMS (ESI): m/z 553.58 [M+H]⁺

### N-((2S)-3-Cyclohexyl-1-oxo-1-((1-oxo-3-(2-oxo-1-azaspiro[4.5]decan-3-yl)propan-2-yl)amino)propan-2-yl)-4-methoxy-1H-indole-2-carboxamide (Compound C193)

To a stirred solution of N-((2S)-3-cyclohexyl-1-((1-hydroxy-3-(2-oxo-1-azaspiro[4.5]decan-3-yl)propan-2-yl)amino)-1-oxopropan-2-yl)-4-methoxy-1H-indole-2-carboxamide (2) (150 mg, 2.715 mmol) was dissolved in DCM (10 mL) was added Dess-Martin periodinane (230 mg, 5.43 mmol) at 0° C. and stirred at room temperature for 3 h. Reaction mixture was diluted with DCM (20 mL) and washed with sat. Hypo solution (3×30 mL) followed by sat. NaHCO₃ solution (3×30 mL). Organic layer was dried over anhydrous Na₂SO₄, filtered and concentrated to get crude compound. The crude compound was purified by combi-flash NP, compound eluted at 5% methanol in dichloromethane to afford N-((2S)-3-cyclohexyl-1-oxo-1-((1-oxo-3-(2-oxo-1-azaspiro[4.5]decan-3-yl)propan-2-yl)amino)propan-2-yl)-4-methoxy-1H-indole-2-carboxamide (Compound C193). TLC system: 10% Methanol in dichloromethane Rf: 0.5 LCMS (ESI): m/z 551.2 (M+H)⁺

### N-((2S)-3-cyclohexyl-1-((4-(cyclopropylamino)-3-hydroxy-4-oxo-1-(2-oxo-1-azaspiro[4.5]decan-3-yl)butan-2-yl)amino)-1-oxopropan-2-yl)-4-methoxy-1H-indole-2-carboxamide (4)

To a stirred solution N-((2S)-3-cyclohexyl-1-oxo-1-((1-oxo-3-(2-oxo-1-azaspiro[4.5]decan-3-yl)propan-2-yl)amino)propan-2-yl)-4-methoxy-1H-indole-2-carboxamide (Compound C193) (370 mg, 0.672 mmol) was dissolved in DCM (10 mL) was added Pyridine (0.1 mL, 2.01 mmol), isocyanocyclopropane (3) (0.07 mL, 1.008 mmol) sequentially at 0° C. followed by TFA (0.15 mL, 1.34 mmol) at 0° C. and stirred at room temperature for 16 h. The progress of the reaction was monitored by TLC and LCMS. Reaction mixture was diluted with dichloromethane and washed with 1N HCl (2×15 mL), brine solution (20 mL). The organic layer was dried over anhydrous Na₂SO₄ and evaporated under reduced pressure to afford crude N-((2S)-3-cyclohexyl-1-((4-(cyclopropylamino)-3-hydroxy-4-oxo-1-(2-oxo-1-azaspiro[4.5]decan-3-yl)butan-2-yl)amino)-1-oxopropan-2-yl)-4-methoxy-1H-indole-2-carboxamide (4). TLC system:

10% Methanol in dichloromethane Rf: 0.5 LCMS (ESI): m/z 635.3[M+H]⁺

### N-((2S)-3-cyclohexyl-1-((4-(cyclopropylamino)-3,4-dioxo-1-(2-oxo-1-azaspiro[4.5]decan-3-yl)butan-2-yl)amino)-1-oxopropan-2-yl)-4-methoxy-1H-indole-2-carboxamide (Compound C190)

To a stirred solution of N-((2S)-3-cyclohexyl-1-((4-(cyclopropylamino)-3-hydroxy-4-oxo-1-(2-oxo-1-azaspiro[4.5]decan-3-yl)butan-2-yl)amino)-1-oxopropan-2-yl)-4-methoxy-1H-indole-2-carboxamide (4) (200 mg, 0.314 mmol) in DCM (10 mL) was added Dess-Martin periodinane (267 mg, 0.629 mmol) at 0° C. and stirred at room temperature for 3 h. The progress of the reaction was monitored by TLC and LCMS. Reaction mixture was filter through celite pad and washed with ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford N-((2S)-3-cyclohexyl-1-((4-(cyclopropylamino)-3,4-dioxo-1-(2-oxo-1-azaspiro[4.5]decan-3-yl)butan-2-yl)amino)-1-oxopropan-2-yl)-4-methoxy-1H-indole-2-carboxamide (Compound C190). TLC system: 10% Methanol in dichloromethane Rf: 0.6 LCMS (ESI): m/z 634.2 (M+H)⁺

### Example 146: Synthesis of Compound C191

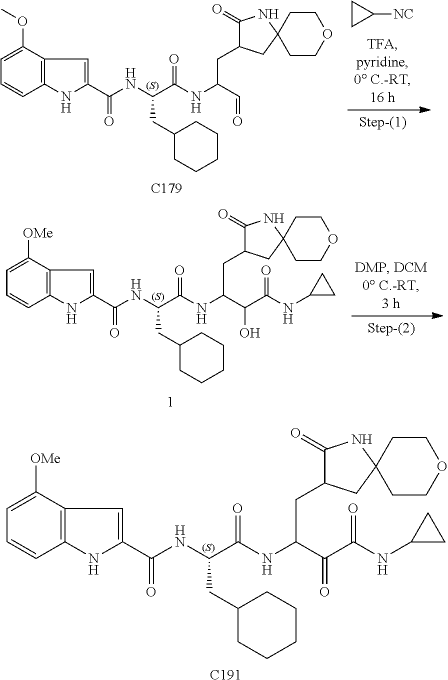

### N-((2S)-3-Cyclohexyl-1-((4-(cyclopropylamino)-3-hydroxy-4-oxo-1-(2-oxo-8-oxa-1-azaspiro[4.5]decan-3-yl)butan-2-yl)amino)-1-oxopropan-2-yl)-4-methoxy-1H-indole-2-carboxamide (2)

To a stirred solution of N-((2S)-3-cyclohexyl-1-oxo-1-((1-oxo-3-(2-oxo-8-oxa-1-azaspiro[4.5]decan-3-yl)propan-2-yl)amino)propan-2-yl)-4-methoxy-1H-indole-2-carboxamide (500 mg, 0.904 mmol) was dissolved in DCM (60 mL), added Pyridine (1.5 mL, 3 vol), isocyanocyclopropane (1) (121 mg, 1.80 mmol) sequentially at 0° C. and stirred for 10 min. To this was added TFA (0.2 mL, 2.5 mmol) at 0° C. and stirred at room temperature for 16 h. The progress of the reaction was monitored by TLC and LCMS. Reaction mixture was quenched with ice water (30 mL) and extracted with dichloromethane (2×30 mL). The organic layer was washed with 1N HCl (3×30 mL), brine solution (3×20 mL). The organic layer was dried over anhydrous Na₂SO₄ and evaporated under reduced pressure to afford crude N-((2S)-3-cyclohexyl-1-((4-(cyclopropylamino)-3-hydroxy-4-oxo-1-(2-oxo-8-oxa-1-azaspiro[4.5]decan-3-yl)butan-2-yl)amino)-1-oxopropan-2-yl)-4-methoxy-1H-indole-2-carboxamide (2). TLC system: 10% Methanol/Dichloromethane Rf: 0.7 LCMS

(ESI): m/z 638.36 [M+H]⁺

### N-((2S)-3-Cyclohexyl-1-((4-(cyclopropylamino)-3,4-dioxo-1-(2-oxo-8-oxa-1-azaspiro[4.5]decan-3-yl)butan-2-yl)amino)-1-oxopropan-2-yl)-4-methoxy-1H-indole-2-carboxamide (Compound C191)

To a stirred solution of N-((2S)-3-cyclohexyl-1-((4-(cyclopropylamino)-3-hydroxy-4-oxo-1-(2-oxo-8-oxa-1-azaspiro[4.5]decan-3-yl)butan-2-yl)amino)-1-oxopropan-2-yl)-4-methoxy-1H-indole-2-carboxamide (2) (400 mg, 0.62 mmol) in DCM (50 mL) was added Dess-Martin periodinane (798 mg, 1.59 mmol) at 0° C. and stirred at room temperature for 3 h. The progress of the reaction was monitored by TLC and LCMS. Reaction mixture was filter through celite pad and washed with Ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford N-((2S)-3-Cyclohexyl-1-((4-(cyclopropylamino)-3,4-dioxo-1-(2-oxo-8-oxa-1-azaspiro[4.5]decan-3-yl)butan-2-yl)amino)-1-oxopropan-2-yl)-4-methoxy-1H-indole-2-carboxamide (Compound C191). TLC system: 10% Methanol/Dichloromethane Rf: 0.8 LCMS (ESI): m/z 636.3 (M+H)⁺

### Example 147: Synthesis of Compounds C192 and C223

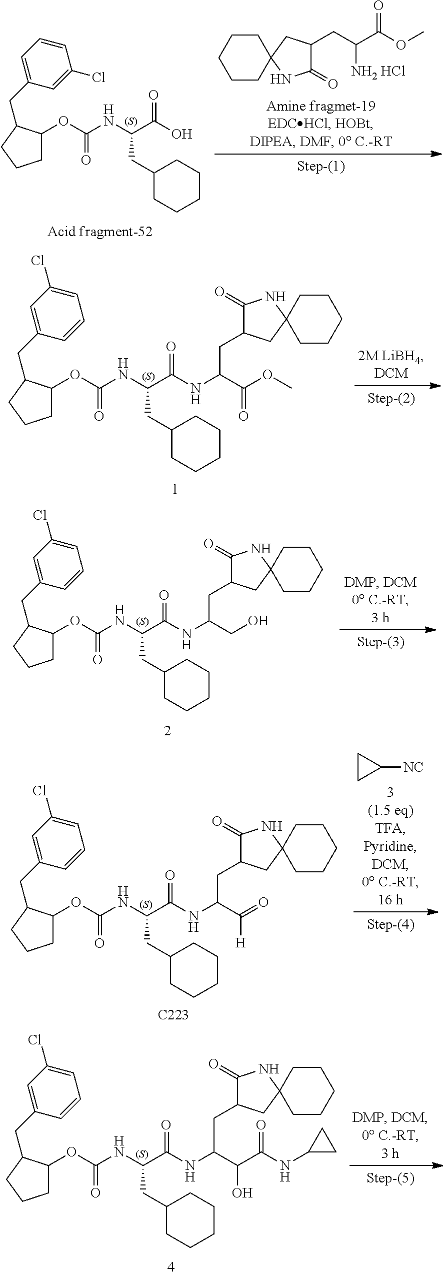

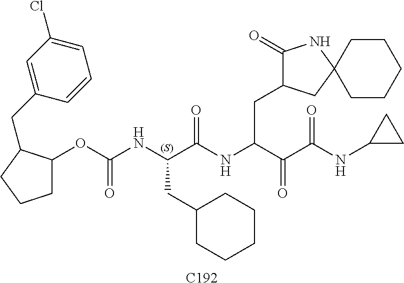

### Methyl 2-((2S)-2-((((2-(3-chlorobenzyl)cyclopentyl)oxy)carbonyl)amino)-3-cyclohexylpropanamido)-3-(2-oxo-1-azaspiro[4.5]decan-3-yl)propanoate (1)

To a stirred solution of (2S)-2-((((2-(3-chlorobenzyl)cyclopentyl)oxy)carbonyl)amino)-3-cyclohexylpropanoic acid (Acid fragment-52) (1.3 g, 3.2 mmol) in DMF (5 mL) at 0° C. was added EDC.HCl (915 mg, 4.7 mmol), HOBT (646 mg, 4.7 mmol), DIPEA (1.7 mL, 3 Vol.) and methyl 2-amino-3-(2-oxo-1-azaspiro[4.5]decan-3-yl)propanoate hydrochloride (Amine fragment-19) (926 mg, 3.2 mmol) simultaneously and stirred at room temperature for 16 h. The progress of the reaction was monitored by TLC and LCMS. After 16 h, reaction mixture was quenched with ice water (20 mL), extracted with ethyl acetate (2×30 mL), the combined organic layer was dried over sodium sulfate and evaporated under reduced pressure. The crude residue was purified by silica gel column by eluting with 5% Methanol in DCM to afford Methyl 2-((2S)-2-((((2-(3-chlorobenzyl)cyclopentyl)oxy)carbonyl)amino)-3-cyclohexylpropanamido)-3-(2-oxo-1-azaspiro[4.5]decan-3-yl)propanoate (1). TLC system: 5% Methanol in DCM R_{f}: 0.5 LCMS (ESI): m/z 644.8 (M+H)⁺

### 2-(3-Chlorobenzyl)cyclopentyl ((2S)-3-cyclohexyl-1-((1-hydroxy-3-(2-oxo-1-azaspiro[4.5]decan-3-yl)propan-2-yl)amino)-1-oxopropan-2-yl)carbamate (2)

To a stirred solution of Methyl 2-((2S)-2-((((2-(3-chlorobenzyl)cyclopentyl)oxy)carbonyl)amino)-3-cyclohexylpropanamido)-3-(2-oxo-1-azaspiro[4.5]decan-3-yl)propanoate (1) (1 g, 1.55 mmol) in DCM (20 mL) was added 2M LiBH₄ in THF (1.55 mL, 3.10 mmol) at 0° C. and the reaction mixture was stirred for 2 h at room temperature. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford 2-(3-chlorobenzyl)cyclopentyl ((2S)-3-cyclohexyl-1-((1-hydroxy-3-(2-oxo-1-azaspiro[4.5]decan-3-yl)propan-2-yl)amino)-1-oxopropan-2-yl)carbamate (2). TLC system: 5% MeOH in DCM R_{f} 0.3 LCMS (ESI): m/z 616.5 (M+H)⁺

### 2-(3-Chlorobenzyl)cyclopentyl ((2S)-3-cyclohexyl-1-oxo-1-((1-oxo-3-(2-oxo-1-azaspiro[4.5]decan-3-yl)propan-2-yl)amino)propan-2-yl)carbamate (Compound C223)

To a stirred solution of 2-(3-chlorobenzyl)cyclopentyl ((2S)-3-cyclohexyl-1-((1-hydroxy-3-(2-oxo-1-azaspiro[4.5]decan-3-yl)propan-2-yl)amino)-1-oxopropan-2-yl)carbamate (2) (850 mg, 1.37 mmol) in ethyl acetate (10 mL) was added Dess-Martin periodinane (1.17 g, 4.13 mmol) at 0° C. and stirred at room temperature for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated afford to 2-(3-Chlorobenzyl)cyclopentyl ((2S)-3-cyclohexyl-1-oxo-1-((1-oxo-3-(2-oxo-1-azaspiro[4.5]decan-3-yl)propan-2-yl)amino)propan-2-yl)carbamate (Compound C223). TLC system: 5% Methanol in DCM R_{f}: 0.4 LCMS (ESI): m/z 614.2 (M+H)⁺

### 2-(3-Chlorobenzyl)cyclopentyl ((2S)-3-cyclohexyl-1-((4-(cyclopropylamino)-3-hydroxy-4-oxo-1-(2-oxo-1-azaspiro[4.5]decan-3-yl)butan-2-yl)amino)-1-oxopropan-2-yl)carbamate (4)

To a stirred solution of 2-(3-Chlorobenzyl)cyclopentyl ((2S)-3-cyclohexyl-1-oxo-1-((1-oxo-3-(2-oxo-1-azaspiro[4.5]decan-3-yl)propan-2-yl)amino)propan-2-yl)carbamate (Compound C223) (600 mg, 1.25 mmol) was dissolved in DCM (60 mL), added Pyridine (1.8 mL, 3 vol), isocyanocyclopropane (3) (126.41 mg, 1.88 mmol) sequentially at 0° C. and stirred for 10 min. To this was added TFA (0.2 mL, 2.5 mmol) at 0° C. and stirred at room temperature for 16 h. The progress of the reaction was monitored by TLC and LCMS. Reaction mixture was quenched with ice water (30 mL) and extracted with dichloromethane (2×30 mL). The organic layer was washed with 1N HCl (3×30 mL), brine solution (3×20 mL). The organic layer was dried over anhydrous Na₂SO₄ and evaporated under reduced pressure to afford crude 2-(3-Chlorobenzyl)cyclopentyl ((2S)-3-cyclohexyl-1-((4-(cyclopropylamino)-3-hydroxy-4-oxo-1-(2-oxo-1-azaspiro[4.5]decan-3-yl)butan-2-yl)amino)-1-oxopropan-2-yl)carbamate (4). TLC system: 5% Methanol/Dichloromethane Rf: 0.5 LCMS (ESI): m/z 700.50 [M+H]⁺

### 2-(3-Chlorobenzyl)cyclopentyl ((2S)-3-cyclohexyl-1-((4-(cyclopropylamino)-3,4-dioxo-1-(2-oxo-1-azaspiro[4.5]decan-3-yl)butan-2-yl)amino)-1-oxopropan-2-yl)carbamate (Compound C192)

To a stirred solution of 2-(3-Chlorobenzyl)cyclopentyl ((2S)-3-cyclohexyl-1-((4-(cyclopropylamino)-3-hydroxy-4-oxo-1-(2-oxo-1-azaspiro[4.5]decan-3-yl)butan-2-yl)amino)-1-oxopropan-2-yl)carbamate (4) (450 mg, 0.79 mmol) in EtOAc (50 mL) was added Dess-Martin periodinane (677 mg, 1.59 mmol) at 0° C. and stirred at room temperature for 3 h. The progress of the reaction was monitored by TLC and LCMS. Reaction mixture was filter through celite pad and washed with Ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford 2-(3-Chlorobenzyl)cyclopentyl ((2S)-3-cyclohexyl-1-((4-(cyclopropylamino)-3,4-dioxo-1-(2-oxo-1-azaspiro[4.5]decan-3-yl)butan-2-yl)amino)-1-oxopropan-2-yl)carbamate (Compound C192). TLC system: 10% Methanol/Dichloromethane Rf: 0.4 LCMS (ESI): m/z 697.2 (M+H)⁺

### Example 148: Synthesis of Compounds C194 and C215

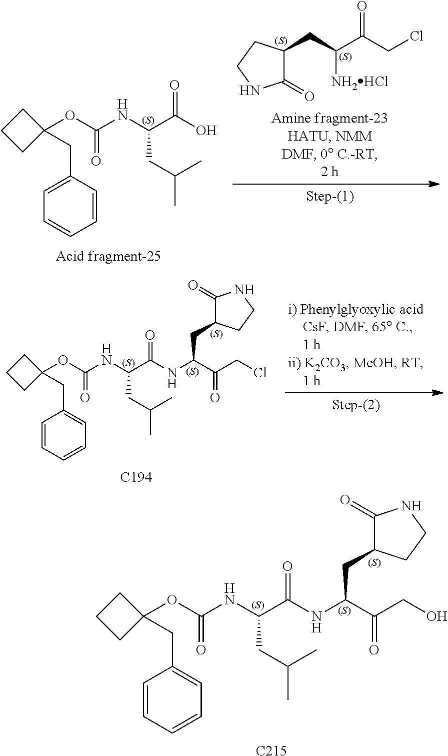

### 1-Benzylcyclobutyl ((S)-1-(((S)-4-chloro-3-oxo-1-((S)-2-oxopyrrolidin-3-yl) butan-2-yl) amino)-4-methyl-1-oxopentan-2-yl) carbamate (Compound C194)

To a stirred solution of ((1-benzylcyclobutoxy)carbonyl)-L-leucine (Acid-fragment-25) (300 mg, 0.940 mmol) in DMF (4 mL) was added HATU (394 mg, 1.410 mmol), NMM (0.2 mL, 1.880 mmol) and (S)-3-((S)-2-amino-4-chloro-3-oxobutyl)pyrrolidin-2-one hydrochloride (Amine-fragment-23) (270 mg, 128 mmol) at 0° C. simultaneously and stirred at room temperature for 16 h. Reaction mixture was diluted with ice water (50 mL), extracted with ethyl acetate (2×30 mL), the organic layer was dried over sodium sulfate and evaporated under reduced pressure to afford crude compound. The crude residue was purified by silica gel column by eluting with 80% ethyl acetate and pet-ether followed by prep HPLC to afford 1-benzylcyclobutyl ((S)-1-(((S)-4-chloro-3-oxo-1-((S)-2-oxopyrrolidin-3-yl) butan-2-yl) amino)-4-methyl-1-oxopentan-2-yl) carbamate (Compound C194). TLC system: 10% Methanol in dichloromethane Rf: 0.3 LCMS (ESI): m/z=506.1 [M+H]⁺

### 1-Benzylcyclobutyl ((S)-1-(((S)-4-hydroxy-3-oxo-1-((S)-2-oxopyrrolidin-3-yl) butan-2-yl) amino)-4-methyl-1-oxopentan-2-yl) carbamate (Compound C215)

To a stirred solution of 1-benzylcyclobutyl ((S)-1-(((S)-4-chloro-3-oxo-1-((S)-2-oxopyrrolidin-3-yl)butan-2-yl)amino)-4-methyl-1-oxopentan-2-yl)carbamate (Compound C194) (280 mg, 0.554 mmol) in DMF (4 mL) was added phenylglyoxylic acid (124 mg, 0.831 mmol) and CsF (167 mg, 1.108 mmol) and heated to 65° C. for 2 h. The progress of the reaction was monitored by TLC and LCMS. Reaction mixture was diluted with ice water (40 mL) and extracted with ethyl acetate (2×30 mL), combined organic layer and washed with cold water (2×30 mL), dried over Na₂SO₄ and concentrated to get crude product, this crude was dissolved in methanol (5 ml) was added K₂CO₃ (7 mg) at 0° C. and stirred for 1 h. The progress of the reaction was monitored by TLC and LCMS. Reaction mixture was concentrated to afford crude. It was purified by prep HPLC to afford 1-benzylcyclobutyl ((S)-1-(((S)-4-hydroxy-3-oxo-1-((S)-2-oxopyrrolidin-3-yl)butan-2-yl)amino)-4-methyl-1-oxopentan-2-yl)carbamate (Compound C215). TLC system: 10% Methanol in DCM Rf: 0.4 LCMS (ESI): m/z 488.3 (M+H)⁺

### Example 149: Synthesis of Compound C195

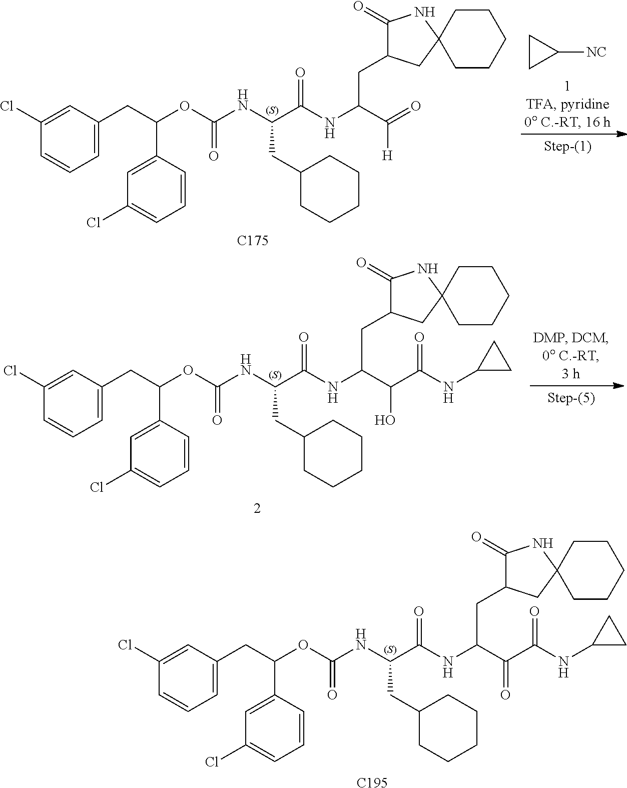

### 2-(3-Chlorobenzyl)cyclo pentyl ((2S)-1-(((2S)-4-(cyclopropylamino)-3-hydroxy-4-oxo-1-((S)-2-oxopyrrolidin-3-yl) butan-2-yl)amino)-4-methyl-1-oxo pentan-2-yl) carbamate (2)

To a stirred solution of 1,2-bis(3-chlorophenyl)ethyl ((2S)-3-cyclohexyl-1-oxo-1-((1-oxo-3-(2-oxo-1-azaspiro[4.5]decan-3-yl)propan-2-yl)amino)propan-2-yl)carbamate (Compound C175) (190 mg, 0.283 mmol) was dissolved in DCM (10 mL) was added Pyridine (0.1 mL, 0.851 mmol), isocyanocyclopropane (1) (0.03 mL, 0.425 mmol) sequentially at 0° C. followed by TFA (0.06 mL, 0.867 mmol) at 0° C. and stirred at room temperature for 16 h. The progress of the reaction was monitored by TLC and LCMS. Reaction mixture was diluted with dichloromethane and washed with 1N HCl (2×15 mL), brine solution (20 mL). The organic layer was dried over anhydrous Na₂SO₄ and evaporated under reduced pressure to afford crude 2-(3-chlorobenzyl)cyclo pentyl ((2S)-1-(((2S)-4-(cyclopropylamino)-3-hydroxy-4-oxo-1-((S)-2-oxopyrrolidin-3-yl) butan-2-yl)amino)-4-methyl-1-oxo pentan-2-yl) carbamate (2). TLC system: 10% Methanol in dichloromethane Rf: 0.5 LCMS (ESI): m/z 754.33[M+H]⁺

### 1,2-Bis(3-chlorophenyl)ethyl ((2S)-3-cyclohexyl-1-((4-(cyclopropylamino)-3,4-dioxo-1-(2-oxo-1-azaspiro[4.5]decan-3-yl)butan-2-yl)amino)-1-oxopropan-2-yl)carbamate (Compound C195)

To a stirred solution of 1,2-bis(3-chlorophenyl)ethyl ((2S)-3-cyclohexyl-1-((4-(cyclopropylamino)-3-hydroxy-4-oxo-1-(2-oxo-1-azaspiro[4.5]decan-3-yl)butan-2-yl)amino)-1-oxopropan-2-yl)carbamate (2) (175 mg, 0.231 mmol) in DCM (10 mL) was added Dess-Martin periodinane (196 mg, 0.4635 mmol) at 0° C. and stirred at room temperature for 3 h. The progress of the reaction was monitored by TLC and LCMS. Reaction mixture was filter through celite pad and washed with ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford 1,2-bis(3-chlorophenyl)ethyl ((2S)-3-cyclohexyl-1-((4-(cyclopropylamino)-3,4-dioxo-1-(2-oxo-1-azaspiro[4.5]decan-3-yl)butan-2-yl)amino)-1-oxopropan-2-yl)carbamate (Compound C195). TLC system: 10% Methanol in dichloromethane Rf: 0.6 LCMS (ESI): m/z 752.3 (M+H)⁺

### Example 150: Synthesis of Compound C196

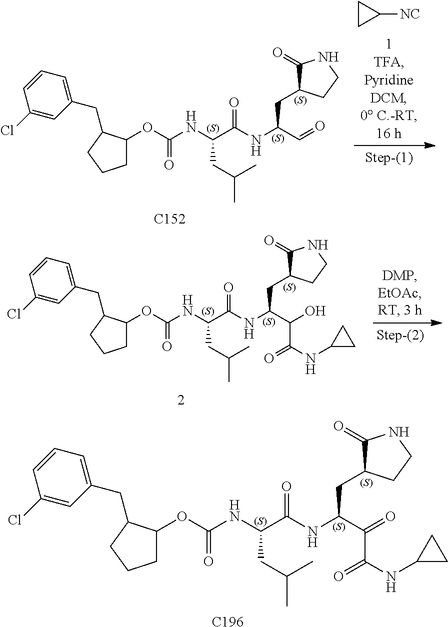

### 2-(3-Chlorobenzyl)cyclopentyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (2)

To a stirred solution of 2-(3-chlorobenzyl) cyclopentyl ((S)-4-methyl-1-oxo-1-(((S)-1-oxo-3-((S)-2-oxopyrrolidin-3-yl)propan-2-yl)amino)pentan-2-yl)carbamate (Compound C152) (350 mg, 0.693 mmol) was dissolved in DCM (10 mL) was added Pyridine (0.75 mL, 3 vol), isocyanocyclopropane (1) (55 mg, 0.831 mmol) sequentially at 0° C. followed by TFA (0.07 mL, 1.386 mmol) at 0° C. and stirred at room temperature for 16 h. The progress of the reaction was monitored by TLC and LCMS. Reaction mixture was diluted with dichloromethane and washed with 1N HCl (2×15 mL), brine solution (20 mL). The organic layer was dried over anhydrous Na₂SO₄ and evaporated under reduced pressure to afford crude 2-(3-chlorobenzyl)cyclo pentyl ((2S)-1-(((2S)-4-(cyclopropylamino)-3-hydroxy-4-oxo-1-((S)-2-oxopyrrolidin-3-yl) butan-2-yl)amino)-4-methyl-1-oxo pentan-2-yl) carbamate (2). TLC system: 10% Methanol in dichloromethane Rf: 0.5 LCMS (ESI): m/z 591.2 [M+H]⁺

### 2-(3-Chlorobenzyl)cyclopentyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C196)

To a stirred solution of 2-(3-chlorobenzyl)cyclopentyl ((2S)-1-(((2S)-4-(cyclo propylamino)-3-hydroxy-4-oxo-1-((S)-2-oxopyrrolidin-3-yl)butan-2-yl)amino)-4-methyl-1-oxopentan-2-yl) carbamate (2) (200 mg, 0.338 mmol) in EtOAc (10 mL) was added Dess-Martin periodinane (431 mg, 1.016 mmol) at 0° C. and stirred at room temperature for 3 h. The progress of the reaction was monitored by TLC and LCMS. Reaction mixture was filter through celite pad and washed with ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford 2-(3-chlorobenzyl)cyclopentyl ((S)-1-(((S)-4-(cyclopropylamino)-3,4-dioxo-1-((S)-2-oxopyrrolidin-3-yl) butan-2-yl)amino)-4-methyl-1-oxopentan-2-yl)carbamate (Compound C196). TLC system: 10% Methanol in dichloromethane Rf: 0.6 LCMS (ESI): m/z 589.1 (M+H)⁺

### Example 151: Synthesis of Compound C197

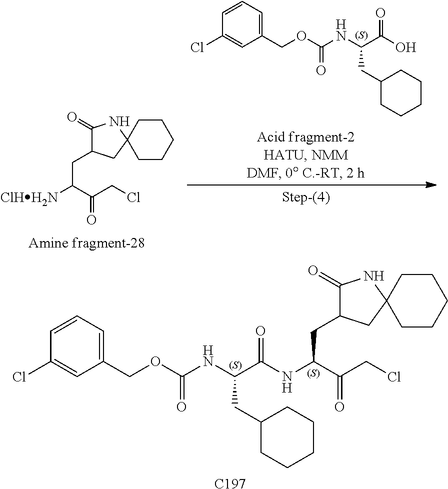

### 3-Chlorobenzyl ((2S)-1-(((2S)-4-chloro-3-oxo-1-(2-oxo-1-azaspiro[4.5]decan-3-yl)butan-2-yl)amino)-3-cyclohexyl-1-oxopropan-2-yl)carbamate (Compound C197)

A solution of (S)-3-((S)-2-amino-4-chloro-3-oxobutyl)pyrrolidin-2-one hydrochloride (Amine fragment-28) (150 mg, 0.442 mmol) and (S)-2-((((3-chlorobenzyl)oxy)carbonyl)amino)-3-cyclohexylpropanoic acid (Acid fragment-2) (164 mg, 0.53 mmol) and in DMF (20 mL) was placed under an atmosphere of N2 and cooled to 0° C. This pale-yellow solution was successively treated with HATU (252 mg, 0.66 mmol) and N-methylmorpholine (0.12 mL, 0.88 mmol). After 1 h, the reaction was quenched with 1:1 ice/sat NaHCO3 (20 mL) and extracted three times with ethyl acetate (30 mL). The combined organics were washed once with brine (100 mL), dried over MgSO4, filtered, and concentrated to get crude. It was purified by prep HPLC to afford to afford 3-chlorobenzyl ((2S)-1-(((2S)-4-chloro-3-oxo-1-(2-oxo-1-azaspiro[4.5]decan-3-yl)butan-2-yl)amino)-3-cyclohexyl-1-oxopropan-2-yl)carbamate (Compound C197). TLC system: 10% MeOH in DCM Rf: 0.6 LCMS (ESI): m/z 594.47 [M+H]⁺

### Example 152: Synthesis of Compound C198

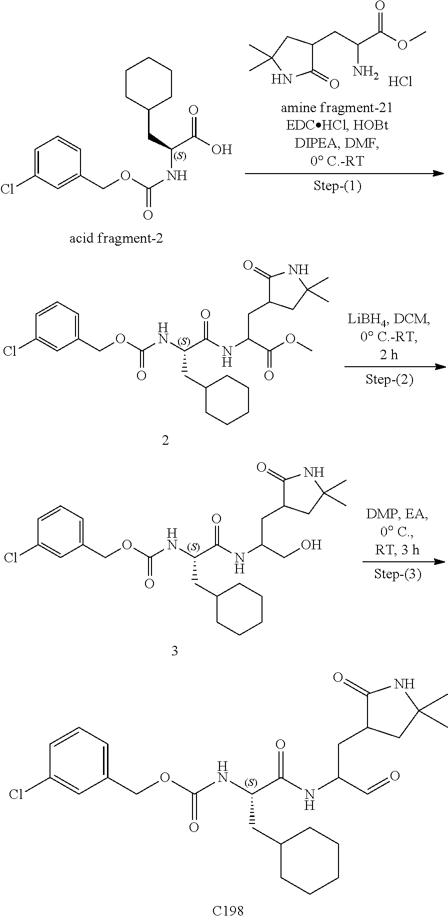

### Methyl 2-((S)-2-((((3-chlorobenzyl)oxy)carbonyl)amino)-3-cyclohexylpropanamido)-3-(5,5-dimethyl-2-oxopyrrolidin-3-yl)propanoate (2)

To a stirred solution of (S)-2-((((3-chlorobenzyl)oxy)carbonyl)amino)-3-cyclohexylpropanoic acid (acid fragment-2) (1 g, 2.94 mmol) in DMF (10 mL) was added EDC.HCl (844 mg, 4.42 mmol), HOBt (596 mg, 4.42 mmol), DIPEA (1.2 mL, 8.84 mmol) followed by methyl 2-amino-3-(5,5-dimethyl-2-oxopyrrolidin-3-yl)propanoate hydrochloride (885 mg, 3.53 mmol) at 0° C. and stirred at room temperature for 16 h. Reaction mixture was quenched with ice water (50 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% methanol in dichloromethane to afford ethyl 2-((S)-2-((((3-chlorobenzyl) oxy) carbonyl) amino)-3-cyclohexylpropanamido)-3-(5,5-dimethyl-2-oxopyrrolidin-3-yl) propanoate (2). TLC system: 5% Methanol in dichloromethane Rf: 0.6 LCMS (ESI): m/z 536.51 [M+H]⁺

### 3-Chlorobenzyl ((2S)-3-cyclohexyl-1-((1-(5,5-dimethyl-2-oxopyrrolidin-3-yl)-3-hydroxypropan-2-yl)amino)-1-oxopropan-2-yl)carbamate (3)

To a stirred solution of methyl 2-((S)-2-((((3-chlorobenzyl)oxy)carbonyl)amino)-3-cyclohexylpropanamido)-3-(5,5-dimethyl-2-oxopyrrolidin-3-yl)propanoate (2) (900 mg, 1.63 mmol) in DCM (10 mL) at 0° C. was added 2M LiBH₄ in THF (1.6 mL, 3.27 mmol) then reaction mixture was stirred for 2 h at room temperature. Reaction mixture was quenched with sat. ammonium chloride solution and extracted with ethyl acetate (2×30 mL). Organic layer was washed with brine solution (30 mL), dried over Na₂SO₄ and concentrated to afford 3-chlorobenzyl ((2S)-3-cyclohexyl-1-((1-(5,5-dimethyl-2-oxopyrrolidin-3-yl)-3-hydroxypropan-2-yl) amino)-1-oxopropan-2-yl) carbamate (3). TLC system: 5% Methanol in DCM Rf: 0.4 LCMS (ESI): m/z 508.55[M+H]⁺

### 3-Chlorobenzyl ((2S)-3-cyclohexyl-1-((1-(5,5-dimethyl-2-oxopyrrolidin-3-yl)-3-oxopropan-2-yl) amino)-1-oxopropan-2-yl) carbamate (Compound C198)

To a stirred solution of 3-chlorobenzyl ((2S)-3-cyclohexyl-1-((1-(5,5-dimethyl-2-oxopyrrolidin-3-yl)-3-hydroxypropan-2-yl)amino)-1-oxopropan-2-yl) carbamate (3) (200 mg, 0.39 mmol) in ethyl acetate (6 mL) was added Dess-Martin periodinane (334 mg, 0.78 mmol) at 0° C. and stirred at room temperature for 3 h. The progress of the reaction was monitored by TLC and LCMS. Reaction mixture was filter through celite pad and washed with ethyl acetate (20 mL) and filtrate was washed with hypo solution (3×15 mL) followed by saturated NaHCO₃ solution (3×15 mL). Organic layer was dried over anhydrous Na₂SO₄, filtered and concentrated to get crude, residue was purified by combi-flash NP, compound eluted at 4% methanol in dichloromethane to afford 3-chlorobenzyl ((2S)-3-cyclohexyl-1-((1-(5,5-dimethyl-2-oxopyrrolidin-3-yl)-3-oxopropan-2-yl) amino)-1-oxopropan-2-yl) carbamate (Compound C198). TLC system: 5% Methanol in dichloromethane Rf: 0.3 LCMS (ESI): m/z 506.1 [M+H]⁺

### Example 153: Synthesis of Compounds C199 and C208

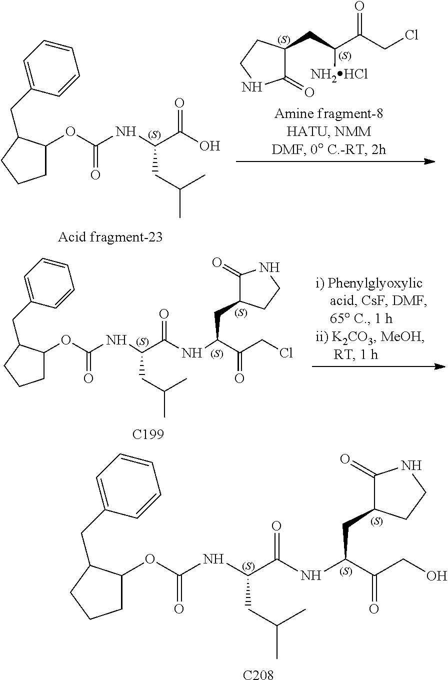

### 2-Benzylcyclopentyl ((S)-1-(((S)-4-chloro-3-oxo-1-((S)-2-oxopyrrolidin-3-yl)butan-2-yl)amino)-4-methyl-1-oxopentan-2-yl)carbamate (Compound C199)

A solution of (S)-3-((S)-2-amino-4-chloro-3-oxobutyl)pyrrolidin-2-one hydrochloride (Amine fragment-23) (540 mg, 2.252 mmol) and (((2-benzylcyclopentyl)oxy)carbonyl)-L-leucine (Acid fragment-23) (500 mg, 1.501 mmol) and in DMF (10 mL) was placed under an atmosphere of N2 and cooled to 0° C. This pale-yellow solution was successively treated with HATU (856 mg, 2.252 mmol) and N-methylmorpholine (0.3 mL, 3.003 mmol). After 1 h, the reaction was quenched with 1:1 ice/sat NaHCO₃ (50 mL) and extracted three times with ethyl acetate (50 mL). The combined organics were washed once with brine (100 mL), dried over Na2SO4, filtered, and concentrated to give a yellow syrup. This material was purified by normal phase chromatography to afford 2-benzylcyclopentyl ((S)-1-(((S)-4-chloro-3-oxo-1-((S)-2-oxopyrrolidin-3-yl)butan-2-yl)amino)-4-methyl-1-oxopentan-2-yl)carbamate (Compound C199). TLC system: 10% MeOH in DCM Rf: 0.6 LCMS (ESI): m/z 520.1 [M+H]⁺

### 2-benzylcyclopentyl ((S)-1-(((S)-4-hydroxy-3-oxo-1-((S)-2-oxopyrrolidin-3-yl)butan-2-yl)amino)-4-methyl-1-oxopentan-2-yl)carbamate (Compound C208)

A solution of 2-benzylcyclopentyl ((S)-1-(((S)-4-chloro-3-oxo-1-((S)-2-oxopyrrolidin-3-yl)butan-2-yl)amino)-4-methyl-1-oxopentan-2-yl)carbamate (200 mg, 0.385 mmol) and benzoylformic acid (87 mg, 0.577 mmol) in DMF (4 mL) was placed under an atmosphere of N2. This clear pale-yellow solution was treated with CsF (116 mg, 0.770 mmol) followed by heating to 65° C. After 4 h, the yellow suspension was cooled to room temperature, diluted with ethyl acetate (60 mL), washed three times water (30 mL), once with brine (30 mL), dried over Na2SO4, filtered, and concentrated to give crude (3S)-3-((2S)-2-((((2-benzylcyclopentyl)oxy)carbonyl)amino)-4-methylpentanamido)-2-oxo-4-((S)-2-oxopyrrolidin-3-yl)butyl 2-oxo-2-phenylacetate as a crude yellow foam. MS (ESI+) for C₃₅H₄₃N₃O₈ m/z 633.3 (M+H)⁺. This crude was taken in methanol (20 mL) was placed under an atmosphere of N₂ and treated with potassium carbonate (10 mg, 0.04 mmol) with vigorous stirring. After 1 h, the volatiles were removed in vacuo (bath <30° C.) to give a crude. This material was purified by normal phase chromatography to afford 2-benzylcyclopentyl ((S)-1-(((S)-4-hydroxy-3-oxo-1-((S)-2-oxopyrrolidin-3-yl)butan-2-yl)amino)-4-methyl-1-oxopentan-2-yl)carbamate (Compound C208) TLC system: 10% MeOH in DCM R_{f}: 0.55 LCMS (ESI): m/z 501.28 [M+H]⁺

### Example 154: Synthesis of Compounds C202 and C210

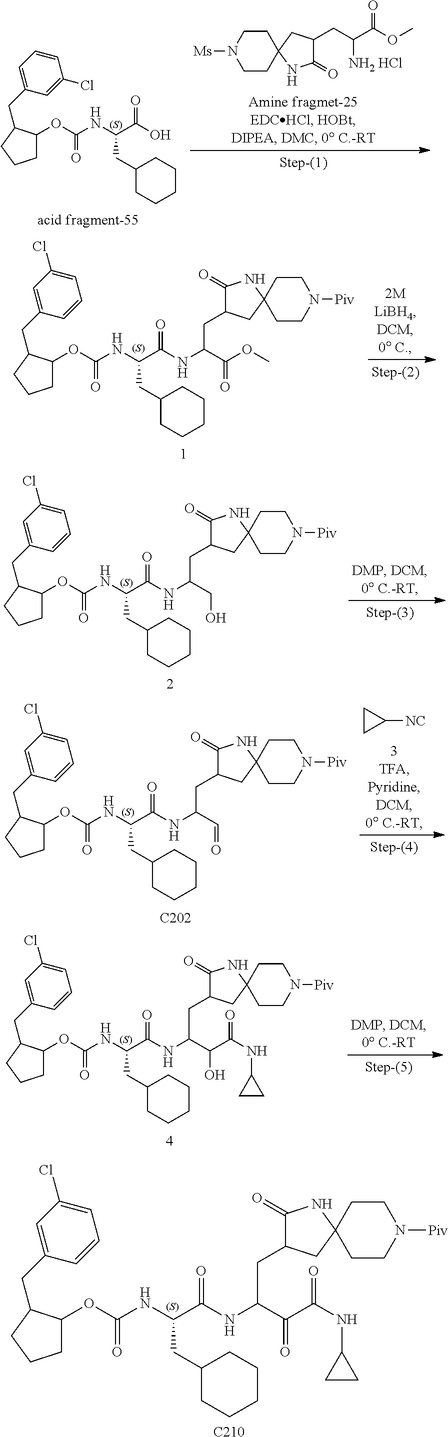

### Methyl 2-((2S)-2-((((2-(3-chlorobenzyl)cyclopentyl)oxy)carbonyl)amino)-3-cyclohexylpropanamido)-3-(2-oxo-8-pivaloyl-1,8-diazaspiro[4.5]decan-3-yl)propanoate (1)

To a stirred solution of (2S)-2-((((2-(3-chlorobenzyl)cyclopentyl)oxy)carbonyl)amino)-3-cyclohexylpropanoic acid (Acid fragment-55) (1 g, 2.45 mmol) in DMF (10 mL) was added EDC.HCl (0.701 g, 3.671 mmol), HOBt (0.49 g, 3.671 mmol), DIPEA (1.2 mL, 7.35 mmol) and methyl 2-amino-3-(2-oxo-8-pivaloyl-1,8-diazaspiro[4.5]decan-3-yl)propanoate hydrochloride (amine-fragment-25) (1 g, 2.94 mmol) at 0° C. simultaneously and stirred at room temperature for 16 h. Reaction mixture was diluted with ice water (50 mL), extracted with ethyl acetate (2×50 mL), the organic layer was dried over sodium sulfate and evaporated under reduced pressure to afford crude compound. The crude residue was purified by silica gel column by eluting with 80% ethyl acetate and pet-ether to afford methyl 2-((2S)-2-((((2-(3-chlorobenzyl)cyclopentyl)oxy)carbonyl)amino)-3-cyclohexylpropanamido)-3-(2-oxo-8-pivaloyl-1,8-diazaspiro[4.5]decan-3-yl)propanoate (1). TLC system: 10% Methanol in dichloromethane Rf: 0.3 LCMS (ESI): m/z=729.71 [M+H]⁺

### 2-(3-Chlorobenzyl) cyclopentyl ((2S)-3-cyclohexyl-1-((1-hydroxy-3-(2-oxo-8-pivaloyl-1,8-diazaspiro[4.5]decan-3-yl)propan-2-yl)amino)-1-oxopropan-2-yl)carbamate (2)

To a stirred solution methyl 2-((2S)-2-((((2-(3-chlorobenzyl)cyclopentyl)oxy)carbonyl) amino)-3-cyclohexylpropanamido)-3-(2-oxo-8-pivaloyl-1,8-diazaspiro[4.5]decan-3-yl) propanoate (1) (1.0 g, 1.371 mmol) in THF (10 mL) was added 2M LiBH₄ in THF (1.3 mL, 2.743 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to afforded 2-(3-chlorobenzyl)cyclopentyl ((2S)-3-cyclohexyl-1-((1-hydroxy-3-(2-oxo-8-pivaloyl-1,8-diazaspiro[4.5]decan-3-yl)propan-2-yl)amino)-1-oxopropan-2-yl)carbamate (2). TLC system: 10% Methanol in DCM Rf: 0.2 LCMS (ESI): m/z 701.24 (M+H)⁺

### 2-(3-Chlorobenzyl) cyclopentyl ((2S)-3-cyclohexyl-1-oxo-1-((1-oxo-3-(2-oxo-8-pivaloyl-1,8-diazaspiro[4.5]decan-3-yl)propan-2-yl)amino)propan-2-yl)carbamate (Compound C202)

To a stirred solution of 2-(3-chlorobenzyl)cyclopentyl ((2S)-3-cyclohexyl-1-((1-hydroxy-3-(2-oxo-8-pivaloyl-1,8-diazaspiro[4.5]decan-3-yl)propan-2-yl)amino)-1-oxopropan-2-yl)carbamate (2) (700 mg, 1.0 mmol) was dissolved in EtOAc (20 mL) was added Dess-Martin periodinane (1.27 g, 3.0 mmol) at 0° C. and stirred at room temperature for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by normal phase purification afforded 2-(3-chlorobenzyl)cyclopentyl ((2S)-3-cyclohexyl-1-oxo-1-((1-oxo-3-(2-oxo-8-pivaloyl-1,8-diazaspiro[4.5]decan-3-yl)propan-2-yl)amino)propan-2-yl)carbamate (Compound C202). TLC system: 5% Methanol in DCM Rf: 0.3 LCMS (ESI): m/z 699.3 (M+H)⁺

### 2-(3-Chlorobenzyl) cyclopentyl ((2S)-3-cyclohexyl-1-((4-(cyclopropylamino)-3-hydroxy-4-oxo-1-(2-oxo-8-pivaloyl-1,8-diazaspiro[4.5]decan-3-yl)butan-2-yl)amino)-1-oxopropan-2-yl)carbamate (4)

To a stirred solution of afforded 2-(3-chlorobenzyl)cyclopentyl ((2S)-3-cyclohexyl-1-oxo-1-((1-oxo-3-(2-oxo-8-pivaloyl-1,8-diazaspiro[4.5]decan-3-yl)propan-2-yl)amino)propan-2-yl)carbamate (Compound C202) (300 mg, 0.42 mmol) in DCM (10 mL) was added isocyanocyclopropane (3) (57 mg, 0.858 mmol), pyridine (0.13 mL, 1.716 mmol) at 0° C. and the reaction mixture was stirred for 30 minutes at 0° C. and added TFA (0.1 mL, 0.858 mmol) and stirred at room temperature for 16 h. The progress of the reaction was monitored by TLC and LCMS. Reaction mixture was acidified with 1N HCl solution (5 mL) and extracted with DCM (2×10 mL). Organic layer was washed with brine solution (5 mL), dried over Na₂SO₄ and concentrated to get crude compound. The crude compound to afford 2-(3-chlorobenzyl)cyclopentyl ((2S)-3-cyclohexyl-1-((4-(cyclopropylamino)-3-hydroxy-4-oxo-1-(2-oxo-8-pivaloyl-1,8-diazaspiro[4.5]decan-3-yl)butan-2-yl)amino)-1-oxopropan-2-yl)carbamate (4). TLC system: 5% Methanol in dichloromethane Rf: 0.2 LCMS (ESI): m/z 784.3 [M+H]⁺

### 2-(3-Chlorobenzyl) cyclopentyl ((2S)-3-cyclohexyl-1-((4-(cyclopropylamino)-3,4-dioxo-1-(2-oxo-8-pivaloyl-1,8-diazaspiro[4.5]decan-3-yl) butan-2-yl) amino)-1-oxopropan-2-yl) carbamate (Compound C210)

To a stirred solution of 2-(3-chlorobenzyl)cyclopentyl ((2S)-3-cyclohexyl-1-((4-(cyclopropylamino)-3-hydroxy-4-oxo-1-(2-oxo-8-pivaloyl-1,8-diazaspiro[4.5]decan-3-yl)butan-2-yl)amino)-1-oxopropan-2-yl)carbamate (4) (200 mg, 0.255 mmol) was dissolved in EtoAc (10 mL) was added Dess-Martin periodinane (324 mg, 0.765 mmol) at 0° C. and stirred at room temperature for 3 h. Reaction mixture was diluted with dichloromethane (10 mL) and washed with sat. Hypo solution (3×20 mL), sat. NaHCO₃ solution (3×20 mL). Organic layer was dried over anhydrous Na₂SO₄, filtered and concentrated to afford crude, this crude was purified by normal phase purification afforded 2-(3-chlorobenzyl)cyclopentyl ((2S)-3-cyclohexyl-1-((4-(cyclopropylamino)-3,4-dioxo-1-(2-oxo-8-pivaloyl-1,8-diazaspiro[4.5]decan-3-yl)butan-2-yl)amino)-1-oxopropan-2-yl)carbamate (Compound C210). TLC system: 5% Methanol in DCM Rf: 0.3 LCMS (ESI): m/z 782.4 (M+H)⁺

### Example 155: Synthesis of Compounds C222 and C205

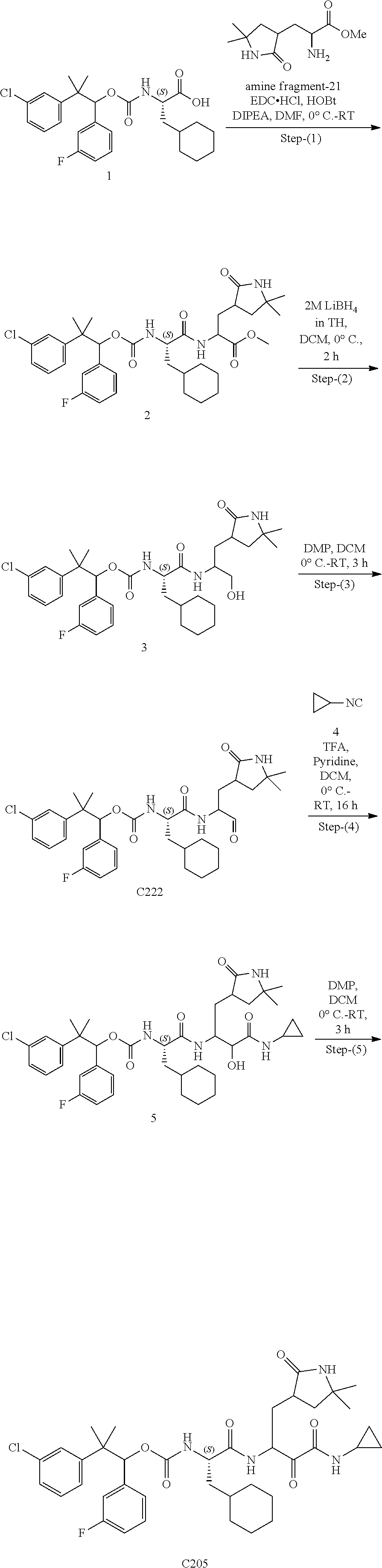

### Methyl 2-((2S)-2-(((2-(3-chlorophenyl)-1-(3-fluorophenyl)-2-methylpropoxy)carbonyl)amino)-3-cyclohexylpropanamido)-3-(5,5-dimethyl-2-oxopyrrolidin-3-yl)propanoate (2)

To a stirred solution of (2S)-2-(((2-(3-chlorophenyl)-1-(3-fluorophenyl)-2-methylpropoxy)carbonyl)amino)-3-cyclohexylpropanoic acid (1) (1.7 g, 3.57 mmol) DMF (10 mL) added EDC.HCl (1 g, 5.36 mmol), HOBt (0.7 g, 5.36 mmol), DIPEA (1.8 mL, 10.73 mmol) and methyl (S)-2-amino-3-((S)-2-oxopyrrolidin-3-yl)propanoate (amine fragment-21) (0.98 g, 3.93 mmol) at 0° C. simultaneously and stirred at room temperature for 16 h. The progress of the reaction was monitored by TLC and LCMS. After 16 h, the reaction mixture was diluted with ice water (50 mL), extracted with ethyl acetate (2×50 mL). The combined organic layer was dried over sodium sulfate, filtered and evaporated under reduced pressure. The crude residue was purified by combi-flash compound eluted at 30% Ethyl acetate in pet ether and after SFC purification to afford Methyl 2-((2S)-2-(((2-(3-chlorophenyl)-1-(3-fluorophenyl)-2-methylpropoxy)carbonyl)amino)-3-cyclohexylpropanamido)-3-(5,5-dimethyl-2-oxopyrrolidin-3-yl)propanoate (2). TLC system: 70% Ethyl acetate in Pet ether Rf: 0.5 LCMS (ESI): m/z 672.60 [M+H]⁺

### 2-(3-chlorophenyl)-1-(3-fluorophenyl)-2-methylpropyl ((2S)-3-cyclohexyl-1-((1-(5,5-dimethyl-2-oxopyrrolidin-3-yl)-3-hydroxypropan-2-yl)amino)-1-oxopropan-2-yl)carbamate (3)

To a stirred solution of methyl 2-((2S)-2-(((2-(3-chlorophenyl)-1-(3-fluorophenyl)-2-methylpropoxy)carbonyl)amino)-3-cyclohexylpropanamido)-3-(5,5-dimethyl-2-oxopyrrolidin-3-yl)propanoate (2) (1.1 g, 1.63 mmol) in THF (10 mL), LiBH₂ (1.6 ml, 3.27 mmol) at 0° C. and continued stirring for 2 h. The progress of the reaction was monitored by TLC and LCMS. Reaction mixture was quenched with sat. Ammonium chloride solution (10 ml) and extracted with ethyl acetate (2×10 mL), dried over sodium sulfate, concentrated under reduced pressure to afford 2-(3-chlorophenyl)-1-(3-fluorophenyl)-2-methylpropyl ((2S)-3-cyclohexyl-1-((1-(5,5-dimethyl-2-oxopyrrolidin-3-yl)-3-hydroxypropan-2-yl)amino)-1-oxopropan-2-yl)carbamate (3). TLC system: 70% Ethyl acetate in Pet ether Rf: 0.2 LCMS (ESI): m/z 644.49 [M+H]⁺

### 2-(3-Chlorophenyl)-1-(3-fluorophenyl)-2-methylpropyl ((2S)-3-cyclohexyl-1-((1-(5,5-dimethyl-2-oxopyrrolidin-3-yl)-3-oxopropan-2-yl)amino)-1-oxopropan-2-yl)carbamate (Compound C222)

To a stirred solution of 3-chlorobenzyl ((2S)-3-cyclohexyl-1-(((2S)-1-hydroxy-3-(8-(methylsulfonyl)-2-oxo-1,8-diazaspiro[4.5]decan-3-yl)propan-2-yl)amino)-1-oxopropan-2-yl)carbamate (3) (230 mg, 0.357 mmol) in ethyl acetate (5 mL) was added Dess-Martin periodinane (379 mg, 0.89 mmol) at 0° C. and stirred at room temperature for 3 h. The progress of the reaction was monitored by TLC and LCMS. After 3 h, the reaction mixture was diluted with ethyl acetate (15 mL) and washed with sat. NaHCO₃ solution (3×20 mL). The organic layer was washed with brine solution (20 mL) and dried over Na₂SO₄ and concentrated to get crude compound. Crude was purified by prep HPLC to afford 3-chlorobenzyl ((2S)-3-cyclohexyl-1-(((2S)-1-(8-(methylsulfonyl)-2-oxo-1,8-diazaspiro[4.5]decan-3-yl)-3-oxopropan-2-yl)amino)-1-oxopropan-2-yl)carbamate (Compound C222). TLC system: 05% Methanol in dichloromethane Rf: 0.2 LCMS (ESI): m/z 642.35 (M+H)⁺

### 2-(3-Chlorophenyl)-1-(3-fluorophenyl)-2-methylpropyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (6)

To a stirred solution of 2-(3-chlorophenyl)-1-(3-fluorophenyl)-2-methylpropyl ((2S)-3-cyclohexyl-1-((1-(5,5-dimethyl-2-oxopyrrolidin-3-yl)-3-oxopropan-2-yl)amino)-1-oxopropan-2-yl)carbamate (Compound C222) (250 mg, 0.436 mmol) and isocyanocyclopropane (4) (58 mg, 0.87 mmol) was dissolved in DCM (6 mL), then added pyridine (0.2 mL), at 0° C. and stirred for 10 min. To this solution was added TFA (99 mg, 0.87 mmol) at 0° C. and stirred at room temperature for 16 h. The progress of the reaction was monitored by TLC and LCMS. Reaction mixture was quenched with ice water (10 mL) and extracted with dichloromethane (2×15 mL). The organic layer was washed with 1N HCl (3×5 mL), brine solution (3×5 mL). The organic layer was dried over anhydrous Na₂SO₄ and evaporated under reduced pressure to afford crude 2-(3-chlorophenyl)-1-(3-fluorophenyl)-2-methylpropyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (5). TLC system: 10% Methanol in dichloromethane Rf: 0.4 LCMS (ESI): m/z 727.47 [M+H]⁺

### 2-(3-Chlorophenyl)-1-(3-fluorophenyl)-2-methylpropyl ((2S)-3-cyclohexyl-1-((4-(cyclopropylamino)-1-(5,5-dimethyl-2-oxopyrrolidin-3-yl)-3,4-dioxobutan-2-yl)amino)-1-oxopropan-2-yl)carbamate (Compound C205)

To a stirred solution of 2-(3-chlorophenyl)-1-(3-fluorophenyl)-2-methylpropyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (5) (250 mg, 0.343 mmol) in ethyl acetate (5 mL) was added Dess-Martin periodinane (364 mg, 0.858 mmol) at 0° C. and stirred at room temperature for 3 h. The progress of the reaction was monitored by TLC and LCMS. After 3 h, the reaction mixture was diluted with ethyl acetate (15 mL) and washed with sat. NaHCO₃ solution (3×20 mL). The organic layer was washed with brine solution (20 mL) and dried over Na₂SO₄ and concentrated to get crude compound. Crude was purified by prep HPLC to afford 3-chlorobenzyl ((2S)-3-cyclohexyl-1-(((2S)-4-(cyclopropylamino)-1-(8-(methylsulfonyl)-2-oxo-1,8-diazaspiro[4.5]decan-3-yl)-3,4-dioxobutan-2-yl)amino)-1-oxopropan-2-yl)carbamate (Compound C205). TLC system: 10% Methanol in dichloromethane Rf: 0.5 LCMS (ESI): m/z 725.01 [M+H]⁺

### Example 156: Synthesis of Compounds C225 and C206

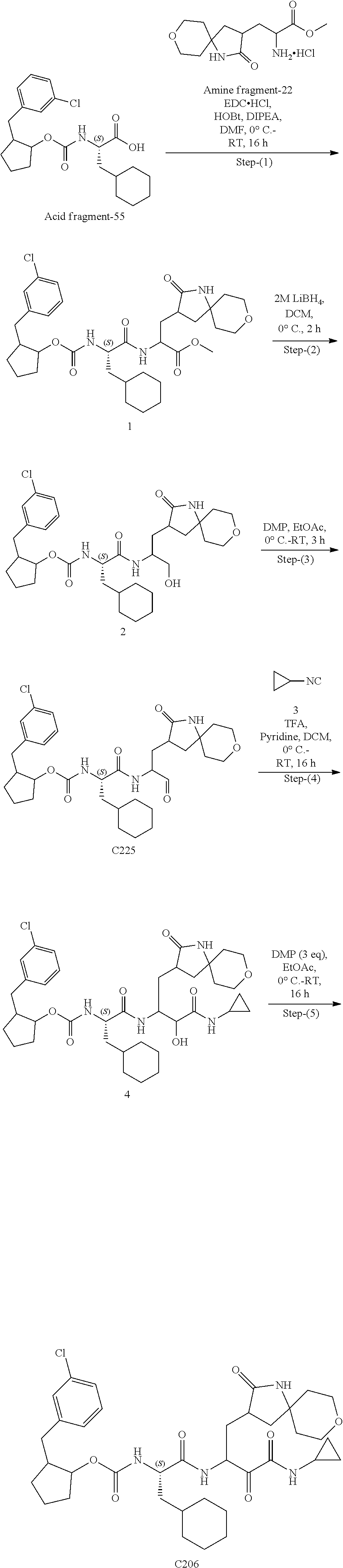

### Methyl 2-((2S)-2-((((2-(3-chlorobenzyl)cyclopentyl)oxy)carbonyl)amino)-3-cyclohexylpropanamido)-3-(2-oxo-8-oxa-1-azaspiro[4.5]decan-3-yl)propanoate (1)

To a stirred solution of (2S)-2-((((2-(3-chlorobenzyl)cyclopentyl)oxy)carbonyl)amino)-3-cyclohexylpropanoic acid (Acid fragment-55) (1.2 g, 2.91 mmol) in DMF (10 mL) at 0° C. was added EDC.HCl (844 mg, 4.4 mmol), HOBT (597 mg, 4.4 mmol), DIPEA (1.56 mL, 8.8 mmol) and methyl (2S)-2-amino-3-(2-oxo-8-oxa-1-azaspiro[4.5]decan-3-yl)propanoate hydrochloride (Amine fragment-22) (1.03 g, 3.5 mmol) simultaneously and stirred at room temperature for 16 h. The progress of the reaction was monitored by TLC and LCMS. After 16 h, reaction mixture was quenched with ice water (30 mL), extracted with ethyl acetate (2×30 mL), the combined organic layer was dried over sodium sulfate and evaporated under reduced pressure. The crude residue was purified by silica gel column by eluting with 70% ethyl acetate in pet ether to methyl 2-((2S)-2-((((2-(3-chlorobenzyl)cyclopentyl)oxy)carbonyl)amino)-3-cyclohexylpropanamido)-3-(2-oxo-8-oxa-1-azaspiro[4.5]decan-3-yl)propanoate (1). TLC system: 5% Methanol in DCM R_{f}: 0.5 LCMS (ESI): m/z 646.4 (M+H)⁺

### 2-(3-chlorobenzyl)cyclopentyl ((2S)-3-cyclohexyl-1-((1-hydroxy-3-(2-oxo-8-oxa-1-azaspiro[4.5]decan-3-yl)propan-2-yl)amino)-1-oxopropan-2-yl)carbamate (2)

To a stirred solution of Methyl 2-((2S)-2-((((2-(3-chlorobenzyl)cyclopentyl)oxy)carbonyl)amino)-3-cyclohexylpropanamido)-3-(2-oxo-8-oxa-1-azaspiro[4.5]decan-3-yl)propanoate (1) (800 mg, 1.20 mmol) in THF (8 mL) was added 2M LiBH₄ in THF (1.23 mL, 2.40 mmol) at 0° C. and the reaction mixture stirred for 2 h at room temperature. The progress of the reaction was monitored by TLC and LCMS. After 2 h, reaction mixture was quenched with water (20 mL) and extracted with ethyl acetate (2×30 mL). Organic layer was washed with brine solution (30 mL), and combined organic layer was dried over Na₂SO₄ and concentrated to afford 2-(3-chlorobenzyl)cyclopentyl ((2S)-3-cyclohexyl-1-((1-hydroxy-3-(2-oxo-8-oxa-1-azaspiro[4.5]decan-3-yl)propan-2-yl)amino)-1-oxopropan-2-yl)carbamate (2). TLC system: 5% MeOH in DCM R_{f} 0.3 LCMS (ESI): m/z 618.6 (M+H)⁺

### 2-(3-chlorobenzyl)cyclopentyl ((2S)-3-cyclohexyl-1-oxo-1-((1-oxo-3-(2-oxo-8-oxa-1-azaspiro[4.5]decan-3-yl)propan-2-yl)amino)propan-2-yl)carbamate (Compound C225)

To a stirred solution of 3-chlorobenzyl 2-(3-chlorobenzyl)cyclopentyl ((2S)-3-cyclohexyl-1-((1-hydroxy-3-(2-oxo-8-oxa-1-azaspiro[4.5]decan-3-yl)propan-2-yl)amino)-1-oxopropan-2-yl)carbamate (2) (400 mg, 0.64 mmol) in ethyl acetate (10 mL) was added Dess-Martin periodinane (823 mg, 1.90 mmol) at 0° C. and stirred at room temperature for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 2-(3-chlorobenzyl)cyclopentyl ((2S)-3-cyclohexyl-1-oxo-1-((1-oxo-3-(2-oxo-8-oxa-1-azaspiro[4.5]decan-3-yl)propan-2-yl)amino)propan-2-yl)carbamate (Compound C225). TLC system: 5% Methanol in DCM R_{f}: 0.4 LCMS (ESI): m/z 616.3 (M+H)⁺

### 2-(3-chlorobenzyl)cyclopentyl ((2S)-3-cyclohexyl-1-((4-(cyclopropylamino)-3-hydroxy-4-oxo-1-(2-oxo-8-oxa-1-azaspiro[4.5]decan-3-yl)butan-2-yl)amino)-1-oxopropan-2-yl)carbamate (4)

To a stirred solution of 2-(3-chlorobenzyl)cyclopentyl ((2S)-3-cyclohexyl-1-oxo-1-((1-oxo-3-(2-oxo-8-oxa-1-azaspiro[4.5]decan-3-yl)propan-2-yl)amino)propan-2-yl)carbamate (Compound C225) (380 mg, 0.64 mmol) was dissolved in DCM (20 mL) added Pyridine (0.24 mL, 2.5 mmol), isocyanocyclopropane (3) (86 mg, 1.29 mmol) sequentially at 0° C. and stirred for 10 min. To this was added TFA (0.10 mL, 1.29 mmol) at 0° C. and stirred at room temperature for 16 h. The progress of the reaction was monitored by TLC and LCMS. Reaction mixture was quenched with ice water (20 mL) and extracted with dichloromethane (2×15 mL). The organic layer was washed with 1N HCl (3×15 mL), brine solution (3×10 mL). The organic layer was dried over anhydrous Na₂SO₄ and evaporated under reduced pressure to afford crude 2-(3-chlorobenzyl)cyclopentyl ((2S)-3-cyclohexyl-1-((4-(cyclopropylamino)-3-hydroxy-4-oxo-1-(2-oxo-8-oxa-1-azaspiro[4.5]decan-3-yl)butan-2-yl)amino)-1-oxopropan-2-yl)carbamate (4). TLC system: 5% Methanol/Dichloromethane Rf: 0.5 LCMS (ESI): m/z 701.3 [M+H]⁺

### 2-(3-Chlorobenzyl)cyclopentyl ((2S)-3-cyclohexyl-1-((4-(cyclopropylamino)-3,4-dioxo-1-(2-oxo-8-oxa-1-azaspiro[4.5]decan-3-yl)butan-2-yl)amino)-1-oxopropan-2-yl)carbamate (Compound C206)

To a stirred solution of 2-(3-chlorobenzyl)cyclopentyl ((2S)-3-cyclohexyl-1-((4-(cyclopropylamino)-3-hydroxy-4-oxo-1-(2-oxo-8-oxa-1-azaspiro[4.5]decan-3-yl)butan-2-yl)amino)-1-oxopropan-2-yl)carbamate (4) (420 mg, 0.61 mmol) in EtOAc (15 mL) was added Dess-Martin periodinane (763 mg, 1.81 mmol) at 0° C. and stirred at room temperature for 16 h. The progress of the reaction was monitored by TLC and LCMS. Reaction mixture was filter through celite pad and washed with Ethyl acetate (35 mL) and filtrate was washed with hypo solution (3×15 mL) followed by saturated NaHCO₃ solution (3×15 mL). Organic layer was dried over anhydrous Na₂SO₄, filtered and concentrated to get crude residue. The crude compound was purified by prep HPLC to afford 2-(3-chlorobenzyl)cyclopentyl ((2S)-3-cyclohexyl-1-((4-(cyclopropylamino)-3,4-dioxo-1-(2-oxo-8-oxa-1-azaspiro[4.5]decan-3-yl)butan-2-yl)amino)-1-oxopropan-2-yl)carbamate (Compound C206). TLC system: 10% Methanol/Dichloromethane Rf: 0.4 LCMS (ESI): m/z 699.3 (M+H)⁺

### Example 157: Synthesis of Compound C207

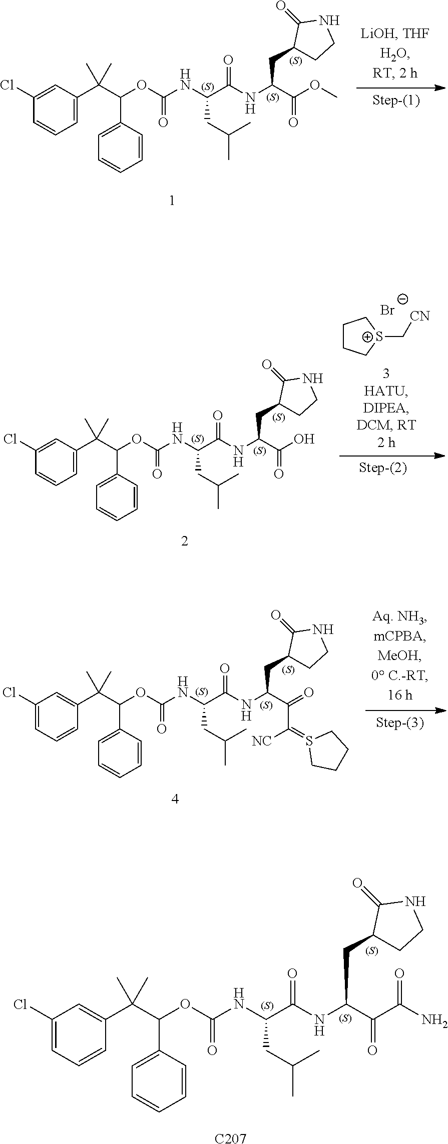

### (2S)-2-((2S)-2-(((2-(3-Chlorophenyl)-2-methyl-1-phenylpropoxy)carbonyl)amino)-4-methylpentanamido)-3-((S)-2-oxopyrrolidin-3-yl)propanoic acid (2)

To a stirred solution of methyl (2S)-2-((2S)-2-(((2-(3-chlorophenyl)-2-methyl-1-phenylpropoxy)carbonyl)amino)-4-methylpentanamido)-3-((S)-2-oxopyrrolidin-3-yl)propanoate (1) (1.3 g, 2.22 mmol) in THF (10 mL), water (3 mL) was added lithium hydroxide (273 mg, 6.65 mmol) at room temperature and stirred at room temperature for 2 h. The progress of the reaction was monitored by TLC and LCMS. The reaction mixture was completely distilled under reduced pressure, crude compound acidified with aq. 1N HCl solution up to pH˜2 and extracted with ethyl acetate (2×30 mL), dried over sodium sulfate, concentrated under reduced pressure to afford (2S)-2-((2S)-2-(((2-(3-chlorophenyl)-2-methyl-1-phenylpropoxy)carbonyl)amino)-4-methylpentanamido)-3-((S)-2-oxopyrrolidin-3-yl)propanoic acid (2). TLC system: 100% EtOAc Rf: 0.1 LCMS (ESI): m/z 572.5 [M+H]⁺

### 2-(3-Chlorophenyl)-2-methyl-1-phenylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4)

To a stirred solution of (2S)-2-((2S)-2-(((2-(3-chlorophenyl)-2-methyl-1-phenylpropoxy)carbonyl)amino)-4-methylpentanamido)-3-((S)-2-oxopyrrolidin-3-yl)propanoic acid (2) (900 mg, 1.57 mmol) DCM (10 mL) was added HATU (1.19 g, 2.89 mmol), DIPEA (0.3 mL, 3 Vol) and 1-(cyanomethyl) tetrahydro-1H-thiophen-1-iumbromide (3) (486 mg, 2.36 mmol) at 0° C. simultaneously and stirred at room temperature for 2 h. Reaction mixture was diluted with ice water (20 mL), extracted with dichloromethane (2×20 mL), dried over sodium sulfate and evaporated under reduced pressure. The crude residue was purified by combi-flash NP, compound eluted at 4% methanol in dichloromethane to afford 2-(3-chlorophenyl)-2-methyl-1-phenylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4). TLC system: 10% Methanol in dichloromethane Rf: 0.3 LCMS (ESI): m/z 681.7 [M+H]⁺

### 2-(3-Chlorophenyl)-2-methyl-1-phenylpropyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C207)

To a stirred solution of ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (200 mg, 0.44 mmol) in methanol (5 mL) was added m-CPBA (189 mg, 1.09 mmol) at 0° C. and the reaction mixture stirred for 2 h at 0° C. To this was added aq ammonia (2 mL) and stirred at room temperature for 16 h. The progress of the reaction was monitored by TLC and LCMS. Reaction mixture was quenched with sat. NaHCO₃ solution (20 mL) and extracted with DCM (2×20 mL). Organic layer was washed with brine solution (30 mL), dried over Na₂SO₄ and concentrated to get crude compound. The crude compound was purified by prep HPLC to afford 2-(3-chlorophenyl)-2-methyl-1-phenylpropyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C207). TLC system: 10% Methanol in dichloromethane Rf: 0.4 LCMS (ESI): m/z 599.1 (M+H)⁺

### Example 158: Synthesis of Compounds C250 and C208

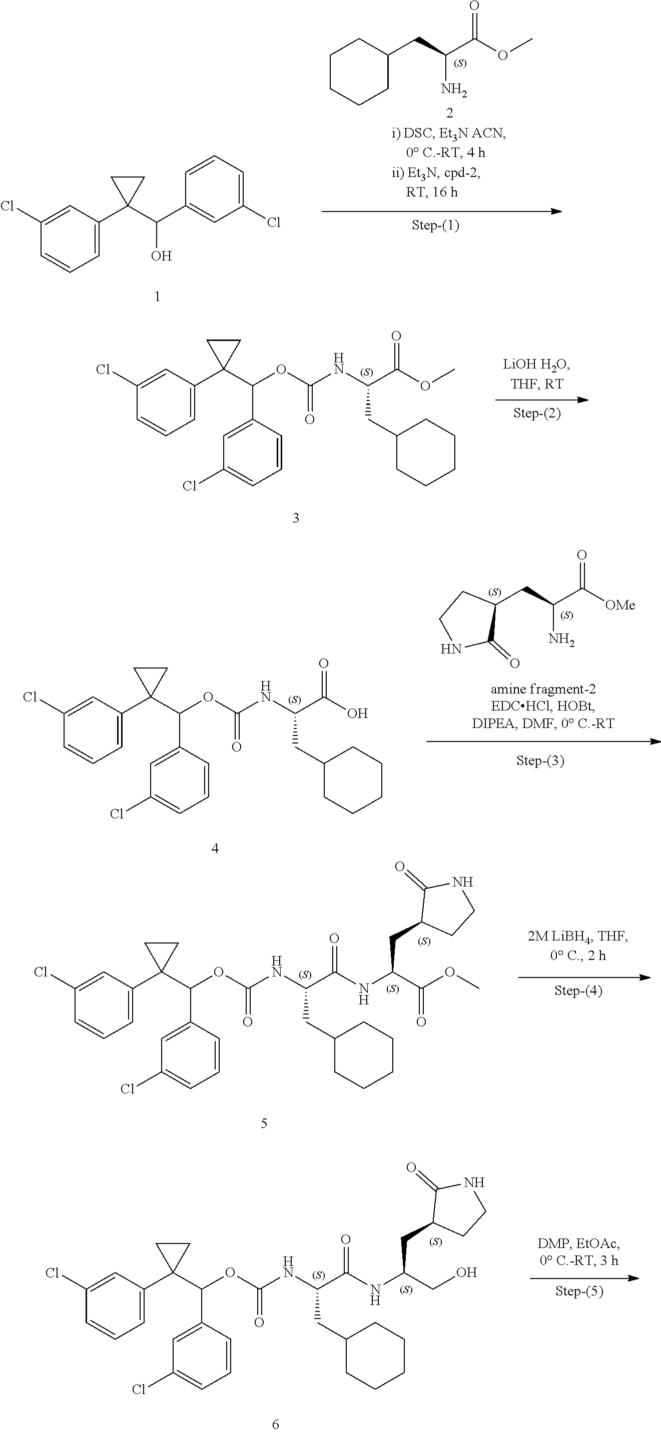

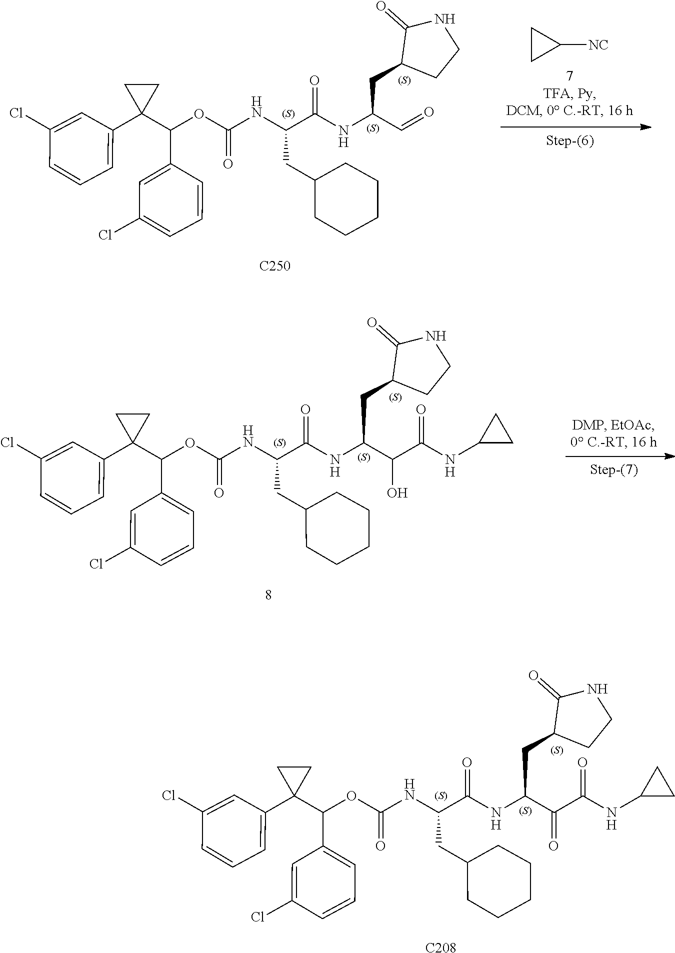

### Methyl (2S)-2-((((3-chlorophenyl)(1-(3-chlorophenyl)cyclopropyl)methoxy)carbonyl)amino)-3-cyclohexylpropanoate (3)

To a stirred solution of (3-chlorophenyl)(1-(3-chlorophenyl)cyclopropyl)methano (1) (2.5 g, 8.51 mmol) in acetonitrile (25 mL) ware added Et₃N (4.16 mL, 29.10 mmol), DSC (5.47 g, 21.01 mmol) at 0° C. and stirred at room temperature for 4 h The progress of the reaction was monitored by TLC. Then added methyl (S)-2-amino-3-cyclohexylpropanoate (2) and stirred at room temperature for 16 h. The progress of the reaction was monitored by TLC and LCMS. Reaction mixture was evaporated under reduced pressure and diluted with water (25 mL) and extracted with ethyl acetate (25 mL), organic layer was dried over sodium sulfate, filtered and evaporated under reduced pressure. The crude residue was purified by combi-flash, compound eluted at 10% ethyl acetate in pet ether to afford methyl (2S)-2-((((3-chlorophenyl)(1-(3-chlorophenyl)cyclopropyl)methoxy)carbonyl)amino)-3-cyclohexylpropanoate (2). TLC system: 10% Ethyl acetate in hexane Rf: 0.4 LCMS (ESI): m/z=0.504.5 [M+H]⁺

### (2S)-2-((((3-chlorophenyl)(1-(3-chlorophenyl)cyclopropyl)methoxy)carbonyl)amino)-3-cyclohexylpropanoic acid (4)

To a stirred solution of methyl (2S)-2-((((3-chlorophenyl)(1-(3-chlorophenyl)cyclopropyl)methoxy)carbonyl)amino)-3-cyclohexylpropanoate (3) (2.5 g, 4.90 mmol) in THF (15 mL), water (10 mL) was added lithium hydroxide (416 mg, 9.92 mmol) at 0° C. and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×30 mL), dried over sodium sulfate, concentrated under reduced pressure to (2S)-2-((((3-chlorophenyl)(1-(3-chlorophenyl)cyclopropyl)methoxy)carbonyl)amino)-3-cyclohexylpropanoic acid (4). TLC system: 50% Ethyl acetate in hexane Rf: 0.2 LCMS (ESI): m/z 490.2 [M+H]⁺

### Methyl (2S)-2-((2S)-2-((((3-chlorophenyl)(1-(3-chlorophenyl)cyclopropyl)methoxy)carbonyl)amino)-3-cyclohexylpropanamido)-3-((S)-2-oxopyrrolidin-3-yl)propanoate (5)

To a stirred solution of (2S)-2-((((3-chlorophenyl)(1-(3-chlorophenyl)cyclopropyl)methoxy)carbonyl)amino)-3-cyclohexylpropanoic acid (4) (1 g, 2.0 mmol) DMF (10 mL) added EDC.HCl (585 mg, 3.01 mmol), HOBt (414 mg, 3.01 mmol), DIPEA (1.09 mL, 6.1 mmol) and methyl (S)-2-amino-3-((S)-2-oxopyrrolidin-3-yl)propanoate (Amine fragment-2) (542 mg, 2.10 mmol) at 0° C. simultaneously and stirred at room temperature for 16 h. The progress of the reaction was monitored by TLC and LCMS. After 16 h, the reaction mixture was diluted with ice water (20 mL), extracted with ethyl acetate (2×30 mL). The combined organic layer was dried over sodium sulfate, filtered and evaporated under reduced pressure. The crude residue was purified by combi-flash compound eluted at 60% Ethyl acetate in pet ether to afford Methyl (2S)-2-((2S)-2-((((3-chlorophenyl)(1-(3-chlorophenyl)cyclopropyl)methoxy)carbonyl)amino)-3-cyclohexylpropanamido)-3-((S)-2-oxopyrrolidin-3-yl)propanoate (5). TLC system: Ethyl acetate Rf: 0.5 LCMS (ESI): m/z 618.5 [M+H]⁺

### (3-Chlorophenyl)(1-(3-chlorophenyl)cyclopropyl)methyl ((S)-3-cyclohexyl-1-(((S)-1-hydroxy-3-((S)-2-oxopyrrolidin-3-yl)propan-2-yl)amino)-1-oxopropan-2-yl)carbamate (6)

To a stirred solution of methyl (2S)-2-((2S)-2-((((3-chlorophenyl)(1-(3-chlorophenyl)cyclopropyl)methoxy)carbonyl)amino)-3-cyclohexylpropanamido)-3-((S)-2-oxopyrrolidin-3-yl)propanoate (5) (900 mg, 1.30 mmol) in THF (9 mL) was added 2M LiBH₄ in THF (1.3 mL, 2.70 mmol) at 0° C. and the reaction mixture stirred for 2 h at 0° C. The progress of the reaction was monitored by TLC and LCMS. Then reaction mixture was quenched with aq. NH4Cl (50 mL) and extracted with ethyl acetate (2×20 mL). Organic layer was washed with brine solution (2×20 mL), dried over Na2SO4 and concentrated to afford (3-chlorophenyl)(1-(3-chlorophenyl)cyclopropyl)methyl ((S)-3-cyclohexyl-1-(((S)-1-hydroxy-3-((S)-2-oxopyrrolidin-3-yl)propan-2-yl)amino)-1-oxopropan-2-yl)carbamate (6). TLC system: Ethyl acetate Rf: 0.2 LCMS (ESI): m/z 630.3 (M+H)⁺

### (3-Chlorophenyl)(1-(3-chlorophenyl)cyclopropyl)methyl ((S)-3-cyclohexyl-1-oxo-1-(((S)-1-oxo-3-((S)-2-oxopyrrolidin-3-yl)propan-2-yl)amino)propan-2-yl)carbamate) (Compound C250)

To a stirred solution of (3-chlorophenyl)(1-(3-chlorophenyl)cyclopropyl)methyl ((S)-3-cyclohexyl-1-(((S)-1-hydroxy-3-((S)-2-oxopyrrolidin-3-yl)propan-2-yl)amino)-1-oxopropan-2-yl)carbamate (6) (200 mg, 0.31 mmol) was dissolved in ethyl acetate (5 mL) was added Dess-Martin periodinane (403 mg, 0.95 mmol) at 0° C. and stirred at room temperature for 3 h. The progress of the reaction was monitored by TLC and LCMS. Reaction mixture was diluted with ethyl acetate (20 mL) and washed with sat. Hypo solution (3×20 mL), sat. NaHCO₃ solution (3×20 mL). Organic layer was dried over anhydrous Na2SO4, filtered and concentrated to afford (3-chlorophenyl)(1-(3-chlorophenyl)cyclopropyl)methyl ((S)-3-cyclohexyl-1-oxo-1-(((S)-1-oxo-3-((S)-2-oxopyrrolidin-3-yl)propan-2-yl)amino)propan-2-yl)carbamate) (Compound C250). TLC system: Ethyl acetate Rf: 0.3 LCMS (ESI): m/z 628.2 (M+H)⁺

### (3-Chlorophenyl)(1-(3-chlorophenyl)cyclopropyl)methyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (8)

To a stirred solution of (3-chlorophenyl)(1-(3-chlorophenyl)cyclopropyl)methyl ((S)-3-cyclohexyl-1-oxo-1-(((S)-1-oxo-3-((S)-2-oxopyrrolidin-3-yl)propan-2-yl)amino)propan-2-yl)carbamate) (Compound C250) (180 mg, 0.28 mmol) in DCM (10 mL) was added isocyanocyclopropane (7) (38 mg, 0.57 mmol), pyridine (0.09. mL, 1.14 mmol) at 0° C. and the reaction mixture was stirred for 30 minutes at 0° C. and added TFA (0.046 mL, 0.57 mmol) and stirred at room temperature for 16 h. The progress of the reaction was monitored by TLC and LCMS. Reaction mixture was acidified with 1N HCl solution (5 mL) and extracted with DCM (2×10 mL). Organic layer was washed with brine solution (5 mL), dried over Na2SO4 and concentrated to get crude compound. The crude compound to afford (3-chlorophenyl)(1-(3-chlorophenyl)cyclopropyl)methyl ((2S)-3-cyclohexyl-1-(((2S)-4-(cyclopropyl amino)-3-hydroxy-4-oxo-1-((S)-2-oxopyrrolidin-3-yl)butan-2-yl)amino)-1-oxopropan-2-yl)carbamate (8). TLC system: 5% Methanol in dichloromethane Rf: 0.2 LCMS (ESI): m/z 713.6 [M+H]⁺

### (3-Chlorophenyl)(1-(3-chlorophenyl)cyclopropyl)methyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C208)

To a stirred solution of (3-chlorophenyl)(1-(3-chlorophenyl)cyclopropyl)methyl ((2S)-3-cyclohexyl-1-(((2S)-4-(cyclopropyl amino)-3-hydroxy-4-oxo-1-((S)-2-oxopyrrolidin-3-yl)butan-2-yl)amino)-1-oxopropan-2-yl)carbamate (8) (200 mg, 0.28 mmol) was dissolved in Ethyl acetate (5 mL) was added Dess-Martin periodinane (297 mg, 0.70 mmol) at 0° C. and stirred at room temperature for 3 h. The progress of the reaction was monitored by TLC and LCMS. Reaction mixture was diluted with Ethyl acetate (15 mL) and washed with sat. Hypo solution (3×20 mL), sat. NaHCO₃ solution (3×20 mL). Organic layer was dried over anhydrous Na2SO4, filtered and concentrated to afford crude, this crude was purified by normal phase purification afforded (3-chlorophenyl)(1-(3-chlorophenyl)cyclopropyl)methyl (3-chlorophenyl)(1-(3-chlorophenyl)cyclopropyl)methyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C208). TLC system: 5% Methanol in DCM Rf: 0.3 LCMS (ESI): m/z 711.3 (M+H)⁺

### Example 159: Synthesis of Compound C209

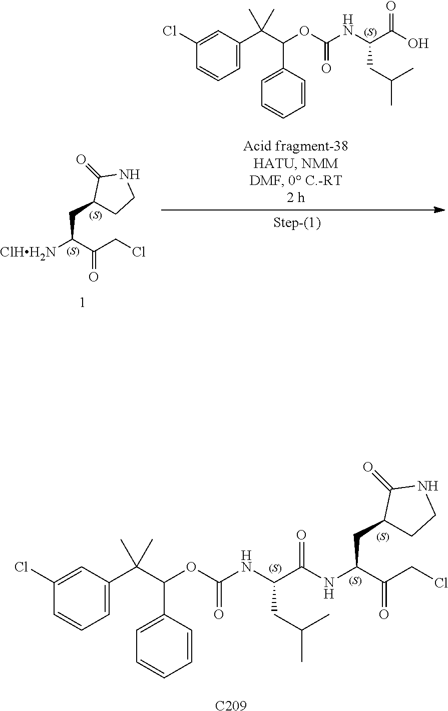

### 2-(3-Chlorophenyl)-2-methyl-1-phenylpropyl ((S)-1-(((S)-4-chloro-3-oxo-1-((S)-2-oxopyrrolidin-3-yl)butan-2-yl)amino)-4-methyl-1-oxopentan-2-yl)carbamate (Compound C209)

A solution of 3-((S)-2-amino-4-chloro-3-oxobutyl)pyrrolidin-2-one hydrochloride (Amine fragment-23) (69.3 mg, 0.28 mmol) and ((2-(3-chlorophenyl)-2-methyl-1-phenylpropoxy)carbonyl)-L-leucine (Acid fragment-38) (100 mg, 0.23 mmol and in DMF (20 mL) was placed under an atmosphere of N2 and cooled to 0° C. This pale-yellow solution was successively treated with HATU (136.62 mg, 0.35 mmol) and N-methylmorpholine (0.1 mL, 0.47 mmol). After 1 h, the reaction was quenched with 1:1 ice/sat NaHCO₃ (20 mL) and extracted three times with ethyl acetate (30 mL). The combined organics were washed once with brine (100 mL), dried over MgSO4, filtered, and concentrated to get crude. It was purified by prep HPLC to afford to afford 2-(3-Chlorophenyl)-2-methyl-1-phenylpropyl ((S)-1-(((S)-4-chloro-3-oxo-1-((S)-2-oxopyrrolidin-3-yl)butan-2-yl)amino)-4-methyl-1-oxopentan-2-yl)carbamate (Compound C209). TLC system: 10% MeOH in DCM Rf: 0.3 LCMS (ESI): m/z 604.2 [M+H]⁺

### Example 160: Synthesis of Compound C214

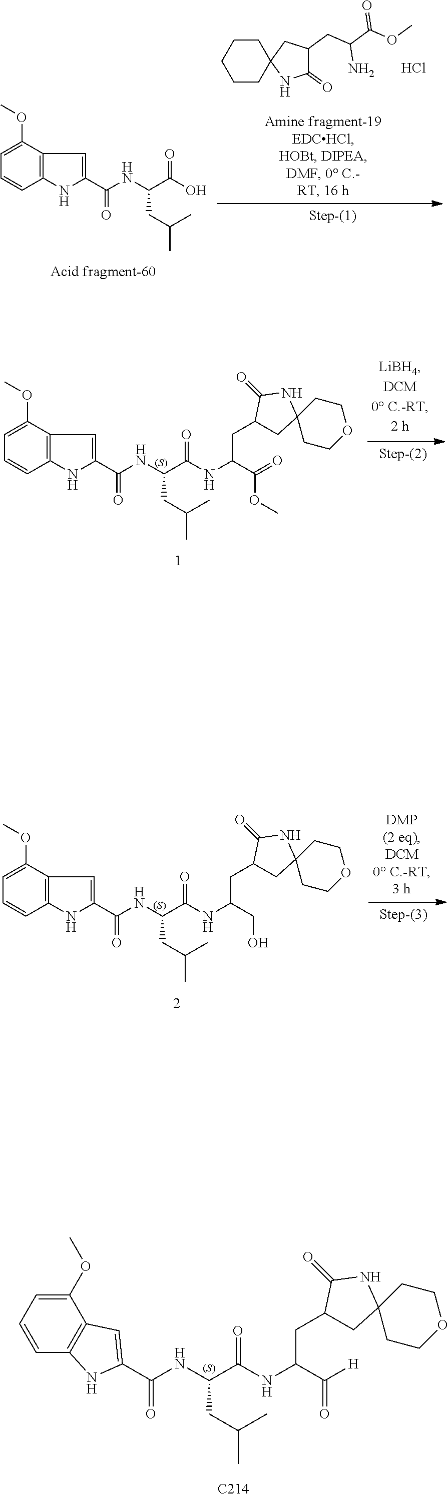

### Methyl 2-((S)-2-(4-methoxy-1H-indole-2-carboxamido)-4-methylpentanamido)-3-(2-oxo-1-azaspiro[4.5]decan-3-yl)propanoate (1)

To a stirred solution of (4-methoxy-1H-indole-2-carbonyl)-D-leucine (Acid fragment-60) (1.5 g, 5 mmol) in DMF (15 mL) at 0° C. was added EDC.HCl (1.42 g, 7.4 mmol), HOBT (1 g, 7.4 mmol) and, DIPEA (2.1 mL, 12.3 mmol) then methyl 2-amino-3-(2-oxo-1-azaspiro[4.5]decan-3-yl)propanoate hydrochloride (Amine fragment-19) (1.7 g, 5.9 mmol) and the resulting reaction mixture was stirred at room temperature for 16 h. The progress of the reaction was monitored by TLC and LCMS. After 16 h, reaction mixture was quenched with ice water (50 mL), extracted with ethyl acetate (2×50 mL), the combined organic layer was dried over sodium sulfate and evaporated under reduced pressure. The crude residue was purified by silica gel column by eluting with 5% methanol in dichloromethane to afford methyl 2-((S)-2-(4-methoxy-1H-indole-2-carboxamido)-4-methylpentanamido)-3-(2-oxo-1-azaspiro[4.5]decan-3-yl)propanoate (1). TLC system: 5% Methanol in DCM R_{f}: 0.5 LCMS (ESI): m/z 541.47 [M+H]⁺

### N-((2S)-1-((1-Hydroxy-3-(2-oxo-1-azaspiro[4.5]decan-3-yl)propan-2-yl)amino)-4-methyl-1-oxopentan-2-yl)-4-methoxy-1H-indole-2-carboxamide (2)

To a stirred solution of methyl 2-((S)-2-(4-methoxy-1H-indole-2-carboxamido)-4-methylpentanamido)-3-(2-oxo-1-azaspiro[4.5]decan-3-yl)propanoate (2) (0.8 g, 1.48 mmol) in DCM (20 mL) was added 2M LiBH₄ in THF (1.5 mL, 2.96 mmol) at 0° C. and the reaction mixture was stirred for 2 h at room temperature. The progress of the reaction was monitored by TLC and LCMS. After 2 h, reaction mixture was quenched with saturated ammonium chloride solution (50 mL) and extracted with DCM (2×50 mL). Combined organic layers were washed with water (50 mL) brine solution (50 mL), and dried over Na₂SO₄ and concentrated to afford N-((2S)-1-((1-hydroxy-3-(2-oxo-1-azaspiro[4.5]decan-3-yl)propan-2-yl)amino)-4-methyl-1-oxopentan-2-yl)-4-methoxy-1H-indole-2-carboxamide (2). TLC system: 10% MeOH in DCM R_{f} 0.3 LCMS (ESI): m/z 513.30 [M+H]⁺

### 4-Methoxy-N-((2S)-4-methyl-1-oxo-1-((1-oxo-3-(2-oxo-1-azaspiro[4.5]decan-3-yl)propan-2-yl)amino)pentan-2-yl)-1H-indole-2-carboxamide (Compound C214)

To a stirred solution of N-((2S)-1-((1-hydroxy-3-(2-oxo-1-azaspiro[4.5]decan-3-yl)propan-2-yl)amino)-4-methyl-1-oxopentan-2-yl)-4-methoxy-1H-indole-2-carboxamide (2) (100 mg, 0.195 mmol) in ethyl acetate (5 mL) was added Dess-Martin periodinane (124 mg, 0.292 mmol) at 0° C. and stirred at room temperature for 16 h. The progress of the reaction was monitored by TLC and LCMS. The reaction mass was filtered through celite pad and the celite pad thoroughly was with ethyl acetate (30 mL). Then the organic layer was washed with 10% sodium thiosulfate solution (2×20 mL) followed by saturated sodium bicarbonate solution (2×20 mL), water (1×20 mL), brine (1×20 mL). Then the organic layer was dried over sodium sulfate and evaporated under vacuum to get 4-methoxy-N-((2S)-4-methyl-1-oxo-1-((1-oxo-3-(2-oxo-1-azaspiro[4.5]decan-3-yl)propan-2-yl)amino)pentan-2-yl)-1H-indole-2-carboxamide (Compound C214). TLC system: 10% Methanol in DCM R_{f}: 0.4 LCMS (ESI): m/z 511.3 [M+H]⁺

### Example 161: Synthesis of Compound C209 and C236

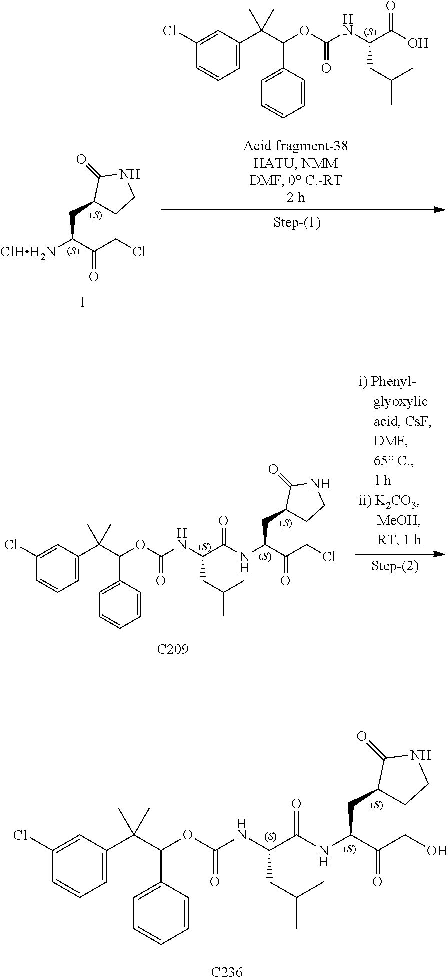

### 2-(3-Chlorophenyl)-2-methyl-1-phenylpropyl ((S)-1-(((S)-4-chloro-3-oxo-1-((S)-2-oxopyrrolidin-3-yl)butan-2-yl)amino)-4-methyl-1-oxopentan-2-yl)carbamate (Compound C209)

A solution of 3-((S)-2-amino-4-chloro-3-oxobutyl)pyrrolidin-2-one hydrochloride (Amine fragment-23) (69.3 mg, 0.28 mmol) and ((2-(3-chlorophenyl)-2-methyl-1-phenylpropoxy)carbonyl)-L-leucine (Acid fragment-38) (100 mg, 0.23 mmol and in DMF (20 mL) was placed under an atmosphere of N2 and cooled to 0° C. This pale-yellow solution was successively treated with HATU (136.62 mg, 0.35 mmol) and N-methylmorpholine (0.1 mL, 0.47 mmol). After 1 h, the reaction was quenched with 1:1 ice/sat NaHCO₃ (20 mL) and extracted three times with ethyl acetate (30 mL). The combined organics were washed once with brine (100 mL), dried over MgSO4, filtered, and concentrated to get crude. It was purified by prep HPLC to afford to afford 2-(3-Chlorophenyl)-2-methyl-1-phenylpropyl ((S)-1-(((S)-4-chloro-3-oxo-1-((S)-2-oxopyrrolidin-3-yl)butan-2-yl)amino)-4-methyl-1-oxopentan-2-yl)carbamate (Compound C209). TLC system: 10% MeOH in DCM Rf: 0.3 LCMS (ESI): m/z 604.2 [M+H]⁺

### 2-(3-Chlorophenyl)-2-methyl-1-phenylpropyl ((S)-1-(((S)-4-hydroxy-3-oxo-1-((S)-2-oxopyrrolidin-3-yl)butan-2-yl)amino)-4-methyl-1-oxopentan-2-yl)carbamate (Compound C236)

To a stirred solution of 2-(3-Chlorophenyl)-2-methyl-1-phenylpropyl ((S)-1-(((S)-4-chloro-3-oxo-1-((S)-2-oxopyrrolidin-3-yl)butan-2-yl)amino)-4-methyl-1-oxopentan-2-yl)carbamate (Compound C209) (550 mg, 0.9097 mmol) Phenylglyoxylicacid (204.86 mg, 1.364 mmol) in DMF (20 mL) was placed under an atmosphere of N2. This clear pale-yellow solution was treated with cesium fluoride (343.41 mg, 2.27 mmol) followed by heating to 65° C. After 4 hr, the now yellow suspension was cooled to room temperature, diluted with ethyl acetate (60 mL), washed three times water (30 mL), once with brine (30 mL), dried over MgSO4, filtered, and concentrated to give crude (3S)-3-((2S)-2-(((2-(3-chlorophenyl)-2-methyl-1-phenylpropoxy)carbonyl)amino)-4-methylpentanamido)-2-oxo-4-((S)-2-oxopyrrolidin-3-yl)butyl 2-oxo-2-phenylacetate as a crude yellow foam. MS (ESI+) for C39H44ClN3O8 m/z 718.75 (M+H)⁺. This crude was taken in methanol (40 mL) was placed under an atmosphere of N2 and treated with cesium carbonate (144.64 mg, 0.446 mmol) with vigorous stirring. After 1 hr the volatiles were removed in vacuo (bath <30° C.) to give a crude. The crude compound was submitted to Prep HPLC afford 2-(3-chlorophenyl)-2-methyl-1-phenylpropyl ((S)-1-(((S)-4-hydroxy-3-oxo-1-((S)-2-oxopyrrolidin-3-yl)butan-2-yl)amino)-4-methyl-1-oxopentan-2-yl)carbamate (Compound C236) TLC system: 10% MeOH in DCM R_{f}: 0.5 LCMS (ESI): m/z 586.2 [M+H]⁺

### Example 162: Synthesis of Compound C211

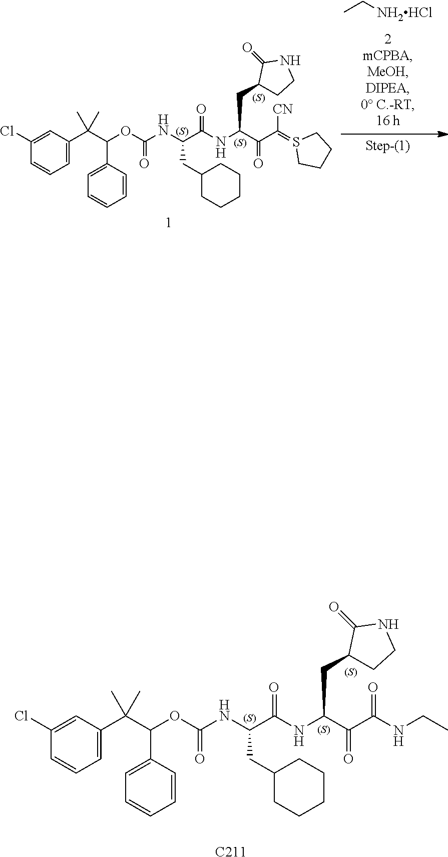

### 2-(3-Chlorophenyl)-2-methyl-1-phenylpropyl ((S)-3-cyclohexyl-1-(((S)-4-(ethylamino)-3,4-dioxo-1-((S)-2-oxopyrrolidin-3-yl) butan-2-yl) amino)-1-oxopropan-2-yl) carbamate (Compound C211)

To a stirred solution of 2-(3-chlorophenyl)-2-methyl-1-phenylpropyl ((S)-1-(((S)-4-cyano-3-oxo-1-((S)-2-oxopyrrolidin-3-yl)-4-(tetrahydro-1l4-thiophen-1-ylidene)butan-2-yl) amino)-3-cyclohexyl-1-oxopropan-2-yl)carbamate (1) (200 mg, 0.27 mmol) in methanol (5 mL) was added m-CPBA (142 mg, 0.831 mmol) at 0° C. and the reaction mixture stirred for 2 h at 0° C. To this was added ethylamine.HCl (600 mg, 7.358 mmol), DIPEA (0.8 mL, 4.49 mmol) and stirred at room temperature for 16 h. The progress of the reaction was monitored by TLC and LCMS. Reaction mixture was quenched with sat. NaHCO₃ solution (20 mL) and extracted with DCM (2×20 mL). Organic layer was washed with brine solution (30 mL), dried over Na₂SO₄ and concentrated to get crude compound. The crude compound was purified by prep HPLC to afford 2-(3-chlorophenyl)-2-methyl-1-phenylpropyl ((S)-3-cyclohexyl-1-(((S)-4-(ethyl amino)-3,4-di oxo-1-((S)-2-oxo pyrrolidin-3-yl)butan-2-yl) amino)-1-oxopropan-2-yl) carbamate (Compound C211). TLC system: 10% Methanol in dichloromethane Rf: 0.2 LCMS (ESI): m/z 667.3 [M+H]⁺

### Example 163: Synthesis of Compound C216

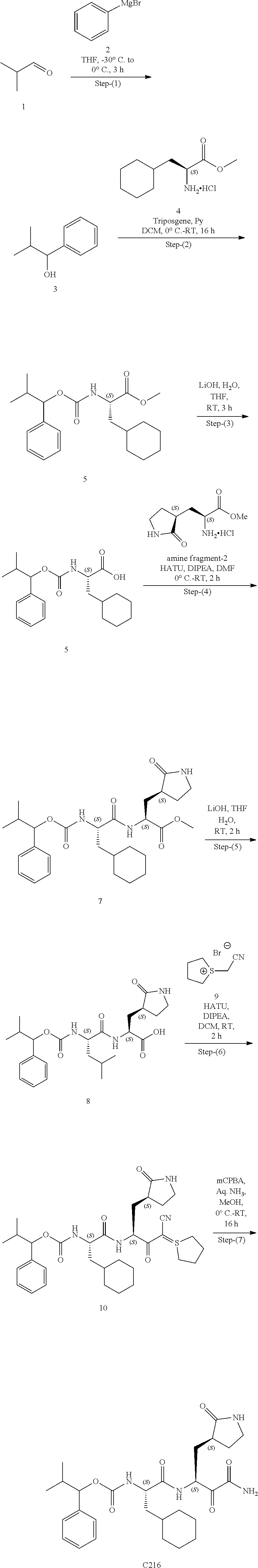

### 2-Methyl-1-phenylpropan-1-ol (3)

To a stirred solution of Phenyl magnesium bromide (2) (139 mL, 138.8 mmol) THF (100 mL) wad added 1-(3-chlorophenyl) isobutyraldehyde (1) (5 g, 69.44 mmol) at −30° C. and stirred at room temperature for 3 h. Reaction progress was monitored by TLC and LCMS. Reaction mixture was quenched with sat. ammonium chloride and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 7% ethyl acetate in hexane to afford 2-methyl-1-phenylpropan-1-ol (3) TLC system: 10% Ethyl acetate in hexane Rf: 0.3 LCMS (ESI): m/z 133.26 [M−OH]⁺

### Methyl (2S)-3-cyclohexyl-2-(((2-methyl-1-phenylpropoxy) carbonyl) amino) propanoate (5)

To a stirred solution of 2-methyl-1-phenylpropan-1-ol (3) (2.0 g, 13.3 mmol) and methyl (S)-2-amino-3-cyclohexylpropanoate hydrochloride (4) (2.95 g, 15.96 mmol) in DCM (30 mL) was added pyridine (6 mL, 3 vol) followed by triphosgene (1.97 g, 6.65 mmol) at 0° C. and stirred at room temperature for 16 h. The progress of the reaction was monitored by TLC and LCMS. Reaction mixture was diluted with DCM and washed with 1N HCl (50 mL) followed by brine (50 mL). Organic layer was dried over sodium sulfate, filtered and evaporated under reduced pressure. The crude residue was purified by combi-flash, compound eluted at 10% ethyl acetate in pet ether to afford methyl (2S)-3-cyclohexyl-2-(((2-methyl-1-phenylpropoxy) carbonyl) amino) propanoate (5). TLC system: 10% Ethyl acetate in hexane Rf: 0.3 LCMS (ESI): m/z 384.21 [M+Na]⁺

### (2S)-3-Cyclohexyl-2-(((2-methyl-1-phenylpropoxy) carbonyl) amino) propanoic acid (6)

To a stirred solution of methyl (2S)-3-cyclohexyl-2-(((2-methyl-1-phenylpropoxy) carbonyl) amino) propanoate (5) (3.0 g, 8.31 mmol) in THF (20 mL), water (10 mL) was added lithium hydroxide (598 mg, 24.93 mmol) at room temperature and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×50 mL), dried over sodium sulfate, concentrated under reduced pressure to afford (2S)-3-cyclohexyl-2-(((2-methyl-1-phenylpropoxy)carbonyl) amino) propanoic acid (6). TLC system: 100% EtOAc Rf: 0.1 LCMS (ESI): m/z 370.19 [M+H]⁺

### Methyl (2S)-2-((2S)-3-cyclohexyl-2-(((2-methyl-1-phenylpropoxy) carbonyl) amino) propanamido)-3-((S)-2-oxopyrrolidin-3-yl) propanoate (7)

To a stirred solution of (2S)-3-cyclohexyl-2-(((2-methyl-1-phenyl propoxy) carbonyl) amino) propanoic acid (6) (2.5 g, 7.21 mmol) DMF (20 mL) added HATU (6.84 g, 18.01 mmol), DIPEA (3.4 mL, 21.6 mmol) and methyl (S)-2-amino-3-((S)-2-oxopyrrolidin-3-yl)propanoate hydrochloride (amine fragment-2) (1.6 g, 8.64 mmol) at 0° C. simultaneously and stirred at room temperature for 16 h. Reaction mixture was quenched with ice water (100 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% methanol in dichloromethane to afford methyl (2S)-2-((2S)-3-cyclohexyl-2-(((2-methyl-1-phenyl propoxy) carbonyl) amino) propan amido)-3-((S)-2-oxopyrrolidin-3-yl) propanoate (7). TLC system: 10% Methanol in dichloromethane Rf: 0.4 LCMS (ESI): m/z 516.47 [M+H]⁺

### (2S)-2-((2S)-3-Cyclohexyl-2-(((2-methyl-1-phenylpropoxy) carbonyl) amino)propanamido)-3-((S)-2-oxopyrrolidin-3-yl)propanoic acid (8)

To a stirred solution of methyl (2S)-2-((2S)-3-cyclohexyl-2-(((2-methyl-1-phenylpropoxy) carbonyl) amino) propanamido)-3-((S)-2-oxo pyrrolidin-3-yl) propanoate (7) (500 mg, 0.97 mmol) in THF (4 mL) and water (2 mL) was added lithium hydroxide (70 mg, 2.91 mmol) at room temperature and stirred at room temperature for 2 h. The progress of the reaction was monitored by TLC and LCMS. Reaction mixture completely distilled under reduced pressure, crude compound acidified with aq. 1N HCl solution up to pH˜2 and extracted with ethyl acetate (2×30 mL), dried over sodium sulfate, concentrated under reduced pressure to afford (2S)-2-((2S)-3-cyclohexyl-2-(((2-methyl-1-phenylpropoxy)carbonyl)amino)propanamido)-3-((S)-2-oxo pyrrolidin-3-yl)propanoic acid (8). TLC system: 100% EtOAc Rf: 0.1 LCMS (ESI): m/z 502.31 [M+H]⁺

### 2-Methyl-1-phenylpropyl ((S)-1-(((S)-4-cyano-3-oxo-1-((S)-2-oxopyrrolidin-3-yl)-4-(tetrahydro-1l4-thiophen-1-ylidene) butan-2-yl) amino)-3-cyclohexyl-1-oxopropan-2-yl) carbamate (10)

To a stirred solution of (2S)-2-((2S)-3-cyclohexyl-2-(((2-methyl-1-phenylpropoxy) carbonyl) amino)propanamido)-3-((S)-2-oxopyrrolidin-3-yl)propanoic acid (8) (400 mg, 0.798 mmol) DCM (10 mL) was added HATU (606 mg, 1.59 mmol), DIPEA (0.37 mL, 2.39 mmol) and 1-(cyanomethyl) tetrahydro-1H-thiophen-1-iumbromide (9) (400 mg, 1.99 mmol) at 0° C. simultaneously and stirred at room temperature for 2 h. Reaction mixture was diluted with ice water (20 mL), extracted with dichloromethane (2×20 mL), dried over sodium sulfate and evaporated under reduced pressure. The crude residue was purified by combi-flash NP, compound eluted at 4% methanol in dichloromethane to afford 2-methyl-1-phenylpropyl ((S)-1-(((S)-4-cyano-3-oxo-1-((S)-2-oxopyrrolidin-3-yl)-4-(tetrahydro-1l4-thiophen-1-ylidene) butan-2-yl) amino)-3-cyclohexyl-1-oxopropan-2-yl) carbamate (10). TLC system: 10% Methanol in dichloromethane Rf: 0.3 LCMS (ESI): m/z 611.41 [M+H]⁺

### 2-Methyl-1-phenylpropyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C216)

To a stirred solution of 2-methyl-1-phenylpropyl ((S)-1-(((S)-4-cyano-3-oxo-1-((S)-2-oxopyrrolidin-3-yl)-4-(tetrahydro-1l4-thiophen-1-ylidene) butan-2-yl) amino)-3-cyclohexyl-1-oxopropan-2-yl) carbamate (10) (350 mg, 0.57 mmol) in methanol (5 mL) was added m-CPBA (296 mg, 1.72 mmol) at 0° C. and the reaction mixture stirred for 2 h at 0° C. and added aq ammonia (2 mL) and stirred at room temperature for 16 h. The progress of the reaction was monitored by TLC and LCMS. Reaction mixture was quenched with sat. NaHCO₃ solution (40 mL) and extracted with DCM (2×20 mL). Organic layer was washed with brine solution (30 mL), dried over Na₂SO₄ and concentrated to get crude compound. The crude compound was purified by prep HPLC to afford 2-methyl-1-phenylpropyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C216). TLC system: 10% Methanol in dichloromethane Rf: 0.4 LCMS (ESI): m/z 529.3 (M+H)⁺

### Example 164: Synthesis of Compound C217

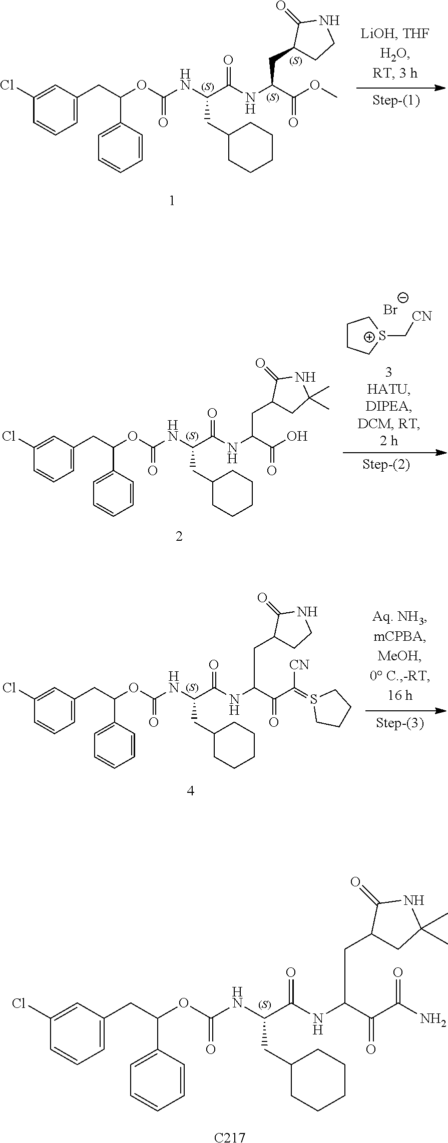

### 2-((2S)-2-(((2-(3-Chlorophenyl)-1-phenylethoxy) carbonyl) amino)-3-cyclohexylpropanamido)-3-(5,5-dimethyl-2-oxopyrrolidin-3-yl) propanoic acid (2)

To a stirred solution of methyl (2S)-2-((2S)-2-(((2-(3-chlorophenyl)-1-phenylethoxy)carbonyl)amino)-3-cyclohexylpropanamido)-3-((S)-2-oxopyrrolidin-3-yl)propanoate (1) (910 mg, 1.45 mmol) in THF (10 mL), water (5 mL) was added lithium hydroxide (178 mg, 4.35 mmol) at room temperature and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×20 mL), dried over sodium sulfate, concentrated under reduced pressure to afford 24(2S)-2-(((2-(3-chlorophenyl)-1-phenylethoxy)carbonyl)amino)-3-cyclohexylpropanamido)-3-(5,5-dimethyl-2-oxopyrrolidin-3-yl)propanoic acid (2). TLC system: 100% Ethyl acetate Rf: 0.1 LCMS (ESI): m/z 612.67 [M+H]⁺

### 2-(3-Chlorophenyl)-1-phenylethyl ((2S)-1-((4-cyano-1-(5,5-dimethyl-2-oxopyrrolidin-3-yl)-3-oxo-4-(tetrahydro-1l4-thiophen-1-ylidene) butan-2-yl) amino)-3-cyclohexyl-1-oxopropan-2-yl) carbamate (4)

To a stirred solution of 2-((2S)-2-(((2-(3-chlorophenyl)-1-phenylethoxy)carbonyl)amino)-3-cyclohexylpropanamido)-3-(5,5-dimethyl-2-oxopyrrolidin-3-yl)propanoic acid (2) (820 mg, 1.339 mmol) in DCM (10 mL) was added HATU (562 mg, 2.00 mmol), DIPEA (0.74 mL, 4.017 mmol) and 1-(cyan methyl)tetra hydro-1H-thiophen-1-iumbromide (3) (414 mg, 2.009 mmol) at 0° C. simultaneously and stirred at room temperature for 2 h. Reaction mixture was diluted with ice water (40 mL), extracted with dichloromethane (2×30 mL), dried over sodium sulfate and evaporated under reduced pressure. The crude residue was purified by combi-flash NP, compound eluted at 5% methanol in dichloromethane to afford 2-(3-chlorophenyl)-1-phenylethyl ((2S)-1-((4-cyano-1-(5,5-dimethyl-2-oxopyrrolidin-3-yl)-3-oxo-4-(tetrahydro-1l4-thiophen-1-ylidene)butan-2-yl)amino)-3-cyclohexyl-1-oxopropan-2-yl)carbamate (4). TLC system: 10% Methanol in dichloromethane Rf: 0.5 LCMS (ESI): m/z 721.49 [M+H]⁺

### 2-(3-Chlorophenyl)-1-phenylethyl ((2S)-1-((4-amino-1-(5,5-dimethyl-2-oxopyrrolidin-3-yl)-3,4-dioxobutan-2-yl) amino)-3-cyclohexyl-1-oxopropan-2-yl) carbamate (Compound C217)

To a stirred solution of 2-(3-chlorophenyl)-1-phenylethyl ((2S)-1-((4-cyano-1-(5,5-dimethyl-2-oxopyrrolidin-3-yl)-3-oxo-4-(tetrahydro-1l4-thiophen-1-ylidene)butan-2-yl)amino)-3-cyclohexyl-1-oxopropan-2-yl)carbamate (4) (300 mg, 0.446 mmol) in methanol (5 mL) was added m-CPBA (215 mg, 1.249 mmol) at 0° C. and the reaction mixture stirred for 2 h at 0° C. To this was added aq ammonia (2 mL) and stirred at room temperature for 16 h. The progress of the reaction was monitored by TLC and LCMS. Reaction mixture was diluted with dichloromethane and washed with sat. NaHCO₃ solution (3×20 mL). Organic layer was washed with brine solution (30 mL), dried over Na₂SO₄ and concentrated to get crude compound. The crude compound was purified by prep HPLC to afford 2-(3-chlorophenyl)-1-phenylethyl ((2S)-1-((4-amino-1-(5,5-dimethyl-2-oxopyrrolidin-3-yl)-3,4-dioxobutan-2-yl)amino)-3-cyclohexyl-1-oxopropan-2-yl)carbamate (Compound C217). TLC system: 10% Methanol in dichloromethane Rf: 0.3 LCMS (ESI): m/z 639.3 [M+H]⁺

### Example 165: Synthesis of Compound C218

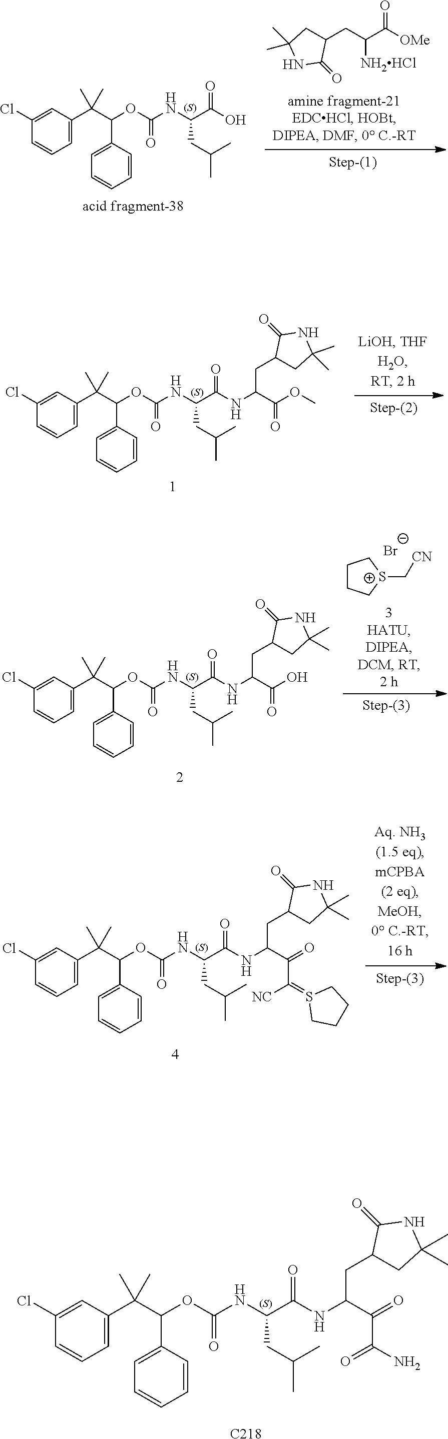

### Methyl 2-((2S)-2-(((2-(3-chlorophenyl)-2-methyl-1-phenylpropoxy) carbonyl) amino)-4-methylpentanamido)-3-(5,5-dimethyl-2-oxopyrrolidin-3-yl) propanoate (1)

To a stirred solution of ((2-(3-chlorophenyl)-2-methyl-1-phenylpropoxy)carbonyl)-L-leucine (acid fragment-38) (1.6 g, 3.83 mmol) DMF (16 mL) added EDC.HCl (1 g, 5.75 mmol), HOBt (0.77 g, 5.75 mmol), DIPEA (1.6 mL, 11.50 mmol) and methyl 2-amino-3-(5,5-dimethyl-2-oxopyrrolidin-3-yl)propanoate hydrochloride (amine fragment-21) (1.15 g, 4.60 mmol) at 0° C. simultaneously and stirred at room temperature for 16 h. Reaction mixture was quenched with ice water (80 mL) and extracted with ethyl acetate (2×50 mL). Combined the organic layer and washed with brine solution (2×50 mL), dried over sodium sulfate and evaporated under reduced pressure. The crude residue was purified by combi-flash NP, compound eluted at 5% methanol in dichloromethane to afford methyl 24(2S)-2-(((2-(3-chlorophenyl)-2-methyl-1-phenylpropoxy) carbonyl) amino)-4-methylpentanamido)-3-(5,5-dimethyl-2-oxopyrrolidin-3-yl) propanoate (1). TLC system: 10% Methanol in dichloromethane Rf: 0.4 LCMS (ESI): m/z 614.48 [M+H]⁺

### 2-((2S)-2-(((2-(3-Chlorophenyl)-2-methyl-1-phenylpropoxy) carbonyl) amino)-4-methylpentanamido)-3-(5,5-dimethyl-2-oxopyrrolidin-3-yl) propanoic acid (2)

To a stirred solution of methyl 2-((2S)-2-(((2-(3-chlorophenyl)-2-methyl-1-phenyl propoxy)carbonyl)amino)-4-methylpentanamido)-3-(5,5-dimethyl-2-oxopyrrolidin-3-yl)propanoate (1) (490 mg, 0.797 mmol) in THF (5 mL), water (3 mL) was added lithium hydroxide (98 mg, 2.393 mmol) at room temperature and stirred at room temperature for 2 h. The progress of the reaction was monitored by TLC and LCMS. Reaction mixture completely distilled under reduced pressure, crude compound acidified with aq. 1N HCl solution up to pH˜2 and extracted with ethyl acetate (2×20 mL), dried over sodium sulfate, concentrated under reduced pressure to afford 2-((2S)-2-(((2-(3-chlorophenyl)-2-methyl-1-phenylpropoxy) carbonyl) amino)-4-methyl pentanamido)-3-(5,5-dimethyl-2-oxopyrrolidin-3-yl)propanoic acid (2). TLC system: 10% Methanol in dichloromethane Rf: 0.2 LCMS (ESI): m/z 586.56 [M+H]⁺

### 2-(3-Chlorophenyl)-2-methyl-1-phenylpropyl ((2S)-1-((4-cyano-1-(5,5-dimethyl-2-oxopyrrolidin-3-yl)-3-oxo-4-(tetrahydro-1l4-thiophen-1-ylidene) butan-2-yl) amino)-4-methyl-1-oxopentan-2-yl) carbamate (3)

To a stirred solution of 2-((2S)-2-(((2-(3-chlorophenyl)-2-methyl-1-phenylpropoxy) carbonyl)amino)-4-methylpentanamido)-3-(5,5-dimethyl-2-oxopyrrolidin-3-yl) propanoic acid (2) (310 mg, 0.516 mmol) in DCM (10 mL) was added HATU (217 mg, 0.774 mmol), DIPEA (0.28 mL, 1.54 mmol) and 1-(cyan methyl)tetra hydro-1H-thiophen-1-iumbromide (3) (159 mg, 0.774 mmol) at 0° C. simultaneously and stirred at room temperature for 2 h. Reaction mixture was diluted with ice water (20 mL), extracted with dichloromethane (2×20 mL), dried over sodium sulfate and evaporated under reduced pressure. The crude residue was purified by combi-flash NP, compound eluted at 5% methanol in dichloromethane to afford 2-(3-chlorophenyl)-2-methyl-1-phenylpropyl ((2S)-1-((4-cyano-1-(5,5-dimethyl-2-oxopyrrolidin-3-yl)-3-oxo-4-(tetrahydro-1l4-thiophen-1-ylidene)butan-2-yl)amino)-4-methyl-1-oxopentan-2-yl)carbamate (4). TLC system: 10% Methanol in dichloromethane Rf: 0.4 LCMS (ESI): m/z 709.52 (M+H)⁺

### 2-(3-Chlorophenyl)-2-methyl-1-phenylpropyl ((2S)-1-((4-amino-1-(5,5-dimethyl-2-oxopyrrolidin-3-yl)-3,4-dioxobutan-2-yl) amino)-4-methyl-1-oxopentan-2-yl)carbamate (Compound C218)

To a stirred solution of 2-(3-chlorophenyl)-2-methyl-1-phenylpropyl ((2S)-1-((4-cyano-1-(5,5-dimethyl-2-oxopyrrolidin-3-yl)-3-oxo-4-(tetrahydro-1l4-thiophen-1-ylidene)butan-2-yl) amino)-4-methyl-1-oxopentan-2-yl)carbamate (3) (240 mg, 0.33 mmol) in methanol (4 mL) was added m-CPBA (116 mg, 0.676 mmol) at 0° C. and the reaction mixture stirred for 2 h at 0° C. To this was added aq ammonia (2 mL) and stirred at room temperature for 16 h. The progress of the reaction was monitored by TLC and LCMS. Reaction mixture was diluted with dichloromethane and washed with sat. NaHCO₃ solution (2×20 mL). Organic layer was washed with brine solution (30 mL), dried over Na₂SO₄ and concentrated to get crude compound. The crude compound was purified by prep HPLC to afford 2-(3-chlorophenyl)-2-methyl-1-phenylpropyl ((2S)-1-((4-amino-1-(5,5-dimethyl-2-oxopyrrolidin-3-yl)-3,4-dioxobutan-2-yl)amino)-4-methyl-1-oxopentan-2-yl) carbamate (Compound C218). TLC system: 10% Methanol in dichloromethane Rf: 0.2 LCMS (ESI): m/z 627.3 [M+H]⁺

### Example 166: Synthesis of Compounds C235 and C219

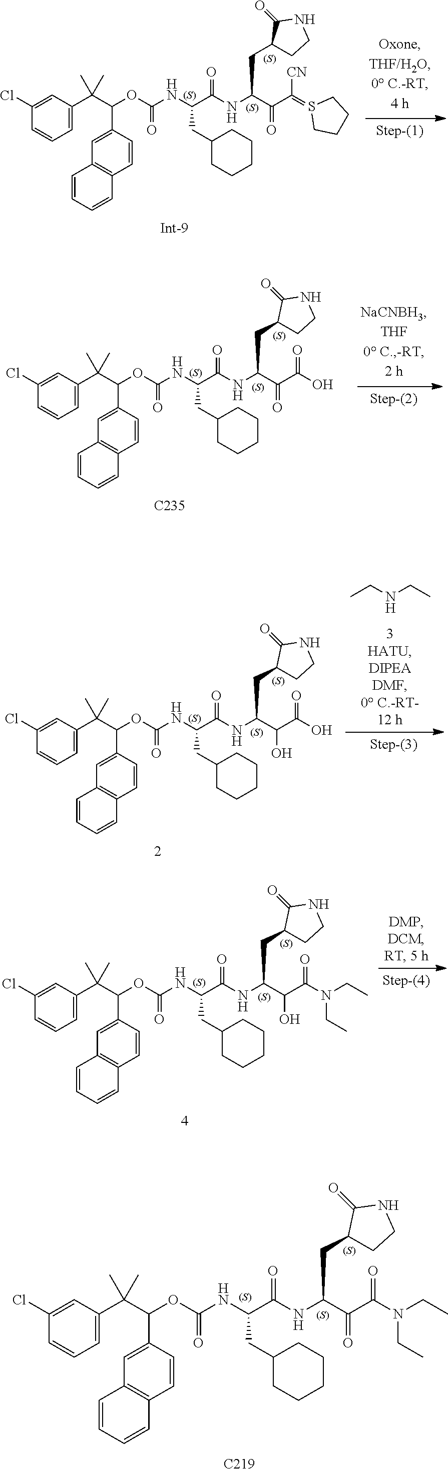

### (3S)-3-((2S)-2-(((2-(3-Chlorophenyl)-2-methyl-1-(naphthalen-2-yl) propoxy) carbonyl)amino)-3-cyclohexylpropanamido)-2-oxo-4-((S)-2-oxopyrrolidin-3-yl)butanoic acid (Compound C235)

To a stirred solution of 2-(3-chlorophenyl)-2-methyl-1-(naphthalen-2-yl)propyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (Int-9) (600 mg, 0.779 mmol) in THF (2.5 mL), water (2.5 mL) was added oxone (717 mg, 2.33 mmol) at room temperature and stirred at room temperature for 2 h. The progress of the reaction was monitored by TLC and LCMS. The reaction mixture was completely distilled under reduced pressure, crude compound acidified with aq. 1N HCl solution up to pH˜2 and extracted with 10% Methanol in DCM (3×15 mL), dried over sodium sulfate, concentrated under reduced pressure to get crude compound was purification by Prep-HPLC to afford (3S)-3-((2S)-2-(((2-(3-chlorophenyl)-2-methyl-1-(naphthalen-2-yl)propoxy)carbonyl)amino)-3-cyclohexyl propanamido)-2-oxo-4-((S)-2-oxopyrrolidin-3-yl)butanoic acid (Compound C235). TLC system: 10% Methanol in DCM Rf: 0.1 LCMS (ESI): m/z 688.59 [M+H]⁺

### (3S)-3-((2S)-2-(((2-(3-Chlorophenyl)-2-methyl-1-(naphthalen-2-yl) propoxy) carbonyl)amino)-3-cyclohexylpropanamido)-2-hydroxy-4-((S)-2-oxopyrrolidin-3-yl)butanoic acid (2)

To a stirred solution of (3S)-3-((2S)-2-(((2-(3-chlorophenyl)-2-methyl-1-(naphthalene-2-yl)propoxy)carbonyl)amino)-3-cyclohexylpropanamido)-2-oxo-4-((S)-2-oxopyrrolidin-3-yl) butanoic acid (Compound C235) (1.0 g, 1.45 mmol) in THF (10 mL) was added NaCNBH₃ (89 mg, 1.45 mmol) was added at 0° C. and stirred at room temperature for 2 h. The progress of the reaction was monitored by TLC and LCMS. Reaction mixture was quenched with ice cold water (10 mL) and extracted with 10% methanol in dichloromethane (3×20 mL) and washed with 1N HCl solution (2×15 mL), followed by brine (20 mL). Organic layer was dried over anhydrous Na₂SO₄ and evaporated under reduced pressure to afford crude was purification by normal phase combi-flash eluted 10% methanol in DCM to afforded (3S)-3-((2S)-2-(((2-(3-chlorophenyl)-2-methyl-1-(naphthalen-2-yl)propoxy)carbonyl)amino)-3-cyclohexylpropanamido)-2-hydroxy-4-((S)-2-oxopyrrolidin-3-yl)butanoic acid (2). TLC system: 15% Methanol in dichloromethane Rf: 0.1 (same RF) LCMS (ESI): m/z 692.36 [M+H]⁺

### 2-(3-Chlorophenyl)-2-methyl-1-(naphthalen-2-yl)propyl((2S)-3-cyclohexyl-1-(((2S)-4-(diethylamino)-3-hydroxy-4-oxo-1-((S)-2-oxopyrrolidin-3-yl)butan-2-yl)amino)-1-oxopropan-2-yl)carbamate (4)

To a stirred solution of (3S)-3-((2S)-2-(((2-(3-chlorophenyl)-2-methyl-1-(naphthalen-2-yl)propoxy)carbonyl)amino)-3-cyclohexylpropanamido)-2-hydroxy-4-((S)-2-oxopyrrolidin-3-yl) butanoic acid (2) (600 mg, 0.868 mmol) in DMF (5 mL) was added HATU (494 mg, 1.302 mmol), DIPEA (0.5 mL, 2.604 mmol) at 0° C. and the reaction mixture stirred for 10 min at 0° C. To this was added di ethyl amine (3) (6 mL) and stirred at room temperature for 16 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 2-(3-chlorophenyl)-2-methyl-1-(naphthalen-2-yl)propyl((2S)-3-cyclohexyl-1-(((2S)-4-(diethylamino)-3-hydroxy-4-oxo-1-((S)-2-oxopyrrolidin-3-yl)butan-2-yl)amino)-1-oxopropan-2-yl)carbamate (4). TLC system: 10% Methanol in dichloromethane Rf: 0.4 LCMS (ESI): m/z 747.5 (M+H)⁺

### 2-(3-Chlorophenyl)-2-methyl-1-(naphthalen-2-yl) propyl ((S)-3-cyclohexyl-1-(((S)-4-(diethylamino)-3,4-dioxo-1-((S)-2-oxopyrrolidin-3-yl)butan-2-yl)amino)-1-oxopropan-2-yl)carbamate (Compound C219)

To a stirred solution of 2-(3-chlorophenyl)-2-methyl-1-(naphthalen-2-yl)propyl((2S)-3-cyclohexyl-1-(((2S)-4-(diethylamino)-3-hydroxy-4-oxo-1-((S)-2-oxopyrrolidin-3-yl)butan-2-yl)amino)-1-oxopropan-2-yl)carbamate (4) (350 mg, 0.469 mmol) was dissolved in Dichloro methane (10 mL) was added Dess-Martin periodinane (995 mg, 2.348 mmol) at 0° C. and stirred at room temperature for 5 h. Reaction mixture was diluted with dichloromethane (20 mL) and washed with sat. Hypo solution (3×20 mL), sat. NaHCO₃ solution (3×20 mL) and brine (1×20 mL). Organic layer was dried over anhydrous Na₂SO₄, filtered and concentrated to afford crude compound. The crude compound was purified by Prep-HPLC purification to afford 2-(3-chlorophenyl)-2-methyl-1-(naphthalen-2-yl)propyl ((S)-3-cyclohexyl-1-(((S)-4-(diethylamino)-3,4-dioxo-1-((S)-2-oxopyrrolidin-3-yl)butan-2-yl)amino)-1-oxopropan-2-yl)carbamate (Compound C219). TLC system: 5% Methanol in dichloromethane Rf: 0.3 LCMS (ESI): m/z 745.3 (M+H)⁺

### Example 167: Synthesis of Compound C220

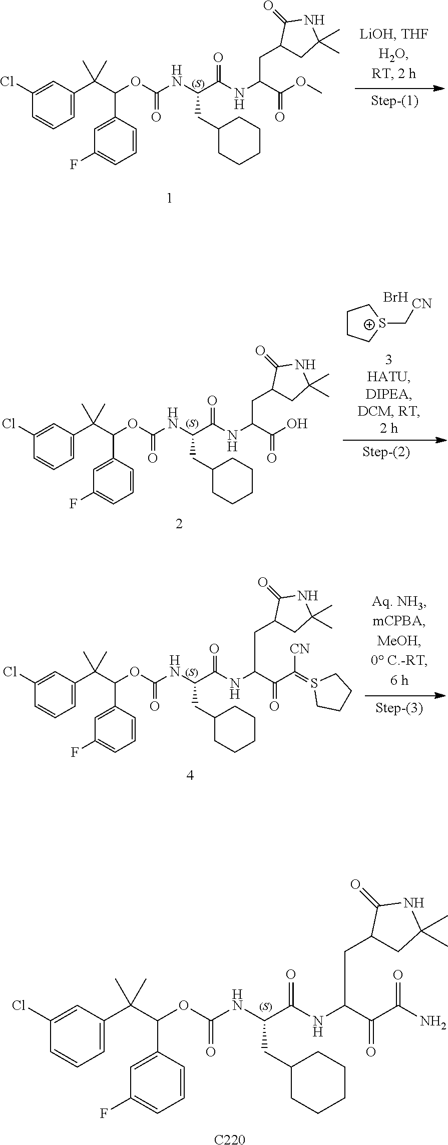

### 2-((2S)-2-(((2-(3-chlorophenyl)-1-(3-fluorophenyl)-2-methylpropoxy)carbonyl)amino)-3-cyclohexylpropanamido)-3-(5,5-dimethyl-2-oxopyrrolidin-3-yl)propanoic acid (2)

To a stirred solution of methyl 2-((2S)-2-(((2-(3-chlorophenyl)-1-(3-fluorophenyl)-2-methylpropoxy)carbonyl)amino)-3-cyclohexylpropanamido)-3-(5,5-dimethyl-2-oxopyrrolidin-3-yl)propanoate (1) (500 mg, 0.74 mmol) in THF (5 mL), water (3 mL) was added lithium hydroxide (60.9 mg, 1.48 mmol) at room temperature and stirred at room temperature for 2 h. The progress of the reaction was monitored by TLC and LCMS. Reaction mixture completely distilled under reduced pressure, crude compound acidified with aq. 1N HCl solution up to pH˜2 and extracted with ethyl acetate (2×30 mL), dried over sodium sulfate, concentrated under reduced pressure to afford (2S)-2-((2S)-2-(((2-(3-chlorophenyl)-2-methyl-1-phenylpropoxy)carbonyl)amino)hexanamido)-3-((S)-2-oxopyrrolidin-3-yl) propanoic acid (2). TLC system: 100% EtOAc Rf: 0.1 LCMS (ESI): m/z 658.45 [M+H]⁺

### 2-(3-Chlorophenyl)-1-(3-fluorophenyl)-2-methylpropyl ((2S)-1-((4-cyano-1-(5,5-dimethyl-2-oxopyrrolidin-3-yl)-3-oxo-4-(tetrahydro-1l4-thiophen-1-ylidene)butan-2-yl)amino)-3-cyclohexyl-1-oxopropan-2-yl)carbamate (4)

To a stirred solution of (2S)-2-((2S)-2-(((2-(3-chlorophenyl)-2-methyl-1-phenylpropoxy)carbonyl)amino)hexanamido)-3-((S)-2-oxopyrrolidin-3-yl) propanoic acid (2) (350 mg, 0.531 mmol) DCM (10 mL) added HATU (302 mg, 0.796 mmol), DIPEA (0.27 mL, 1.59 mmol) and 1-(cyanomethyl) tetrahydro-1H-thiophen-1-iumbromide (3) (172 mg, 0.796 mmol) at 0° C. simultaneously and stirred at room temperature for 2 h. Reaction mixture was diluted with ice water (20 mL), extracted with dichloromethane (2×20 mL), dried over sodium sulfate and evaporated under reduced pressure. The crude residue was purified by combi-flash NP, compound eluted at 4% methanol in dichloromethane to afford 2-(3-chlorophenyl)-1-(3-fluorophenyl)-2-methylpropyl ((2S)-1-((4-cyano-1-(5,5-dimethyl-2-oxopyrrolidin-3-yl)-3-oxo-4-(tetrahydro-1l4-thiophen-1-ylidene)butan-2-yl)amino)-3-cyclohexyl-1-oxopropan-2-yl)carbamate (4). TLC system: 10% Methanol in dichloromethane Rf: 0.3 LCMS (ESI): m/z 767.55 [M+H]⁺

### 2-(3-Chlorophenyl)-1-(3-fluorophenyl)-2-methylpropyl ((2S)-1-((4-amino-1-(5,5-dimethyl-2-oxopyrrolidin-3-yl)-3,4-dioxobutan-2-yl)amino)-3-cyclohexyl-1-oxopropan-2-yl)carbamate (Compound C220)

To a stirred solution of 2-(3-chlorophenyl)-2-methyl-1-phenylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-1-oxohexan-2-yl)carbamate (4) (200 mg, 0.26 mmol) in methanol (5 mL) was added m-CPBA (112 mg, 0.65 mmol) at 0° C. and the reaction mixture stirred for 2 h at 0° C. and added aq ammonia (1 mL) and stirred at room temperature for 16 h. The progress of the reaction was monitored by TLC and LCMS. Reaction mixture was quenched with sat. NaHCO₃ solution (40 mL) and extracted with DCM (2×20 mL). Organic layer was washed with brine solution (30 mL), dried over Na₂SO₄ and concentrated to get crude compound. The crude compound was purified by prep HPLC to afford 2-(3-chlorophenyl)-1-(3-fluorophenyl)-2-methylpropyl ((2S)-1-((4-amino-1-(5,5-dimethyl-2-oxopyrrolidin-3-yl)-3,4-dioxobutan-2-yl)amino)-3-cyclohexyl-1-oxopropan-2-yl)carbamate (Compound C220). TLC system: 10% Methanol in dichloromethane Rf: 0.4 LCMS (ESI): m/z 685.2 (M+H)⁺

### Example 168: Synthesis of Compound C221

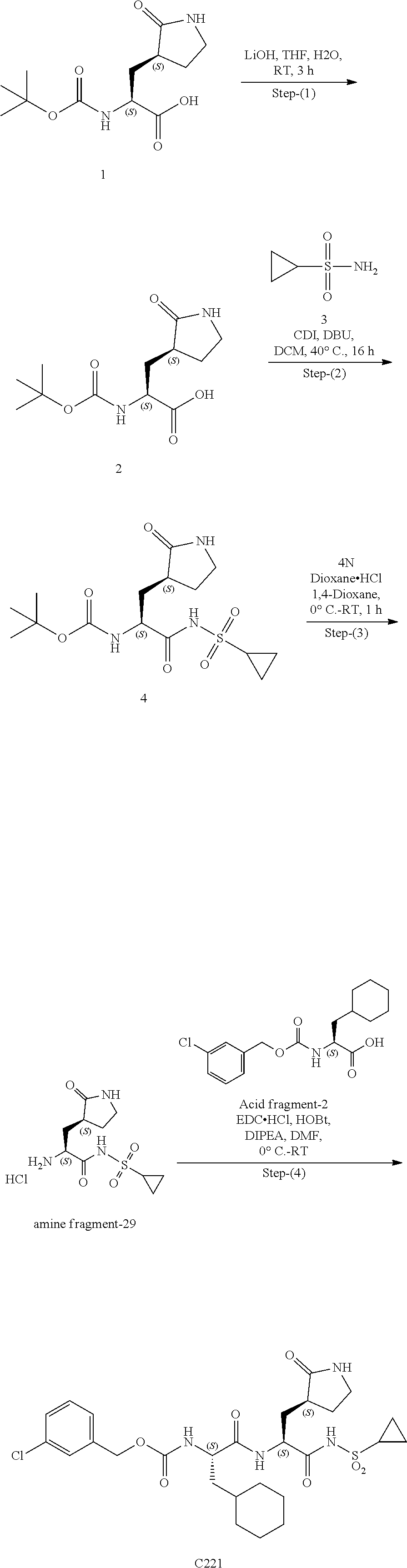

### (S)-2-((tert-butoxycarbonyl) amino)-3-((S)-2-oxopyrrolidin-3-yl) propanoic acid (2)

To a stirred solution of methyl (S)-2-((tert-butoxycarbonyl)amino)-3-((S)-2-oxopyrrolidin-3-yl)propanoate (1) (3 g, 10.48 mmol) in THF (15 mL), water (5 mL) was added lithium hydroxide (800 mg, 20.97 mmol) at room temperature and stirred at room temperature for 2 h. The progress of the reaction was monitored by TLC and LCMS. The reaction mixture was completely distilled under reduced pressure, crude compound acidified with aq. 1N HCl solution up to pH˜2 and extracted with ethyl acetate (2×30 mL), dried over sodium sulfate, concentrated under reduced pressure to afford (S)-2-((tert-butoxycarbonyl)amino)-3-((S)-2-oxopyrrolidin-3-yl)propanoic acid (2). TLC system: 100% EtOAc Rf: 0.1 LCMS (ESI): m/z 273.20 [M+H]⁺

### Tert-butyl ((S)-1-(cyclopropanesulfonamido)-1-oxo-3-((S)-2-oxopyrrolidin-3-yl) propan-2-yl) carbamate (3)

To a stirred solution of (S)-2-((tert-butoxycarbonyl) amino)-3-((S)-2-oxopyrrolidin-3-yl) propanoic acid (2) (1 g, 3.67 mmol) in DCM was added CDI (1.1 g, 7.35 mmol) at room temperature and stirred at 40° C. for 2 h. After that added cyclopropanesulfonamide (889 mg, 7.35 mmol), DBU (1.1 mL, 7.35 mmol) and stirred for 16 h. The progress of the reaction was monitored by TLC and LCMS. Reaction mixture was directly evaporated under reduced pressure. The crude residue was purified by combi-flash NP, compound eluted at 15% methanol in dichloromethane to afford Tert-butyl ((S)-1-(cyclopropanesulfonamido)-1-oxo-3-((S)-2-oxopyrrolidin-3-yl) propan-2-yl) carbamate (4). TLC system: 10% Methanol in DCM R_{f}: 0.1 LCMS (ESI): m/z 376.46 (M+H)⁺

### (S)-2-Amino-N-(cyclopropylsulfonyl)-3-((S)-2-oxopyrrolidin-3-yl) propanamide hydrochloride (amine fragment-29)

To a stirred solution of tert-butyl ((S)-1-(cyclopropanesulfonamido)-1-oxo-3-((S)-2-oxopyrrolidin-3-yl)propan-2-yl)carbamate (4) (1.2 g, 3.2 mmol) in 1,4 dioxane (10 mL) at 0° C. was added drop wise 4N HCl in dioxane (15 mL) and the reaction mixture was stirred at room temperature for 2 h. The progress of the reaction was monitored by TLC. The reaction mixture was evaporated under reduced pressure to obtained crude compound, the resulting crude triturated with diethyl ether to afford (S)-2-amino-N-(cyclopropylsulfonyl)-3-((S)-2-oxopyrrolidin-3-yl) propanamide hydrochloride (amine fragment-29). TLC system: 10% Methanol in DCM R_{f}: 0.1 LCMS (ESI): m/z 276.21 (M+H)⁺

### 3-Chlorobenzyl ((S)-3-cyclohexyl-1-(((S)-1-(cyclopropanesulfonamido)-1-oxo-3-((S)-2-oxopyrrolidin-3-yl) propan-2-yl) amino)-1-oxopropan-2-yl) carbamate (Compound C221)

To a stirred solution of (S)-2-((((3-chlorobenzyl)oxy)carbonyl)amino)-3-cyclohexyl propanoic acid (acid fragment-2) (150 mg, 0.442 mmol) DMF (6 mL) added EDC.HCl (126 mg, 0.663 mmol), HOBt (89 mg, 0.663 mmol), DIPEA (0.2 mL, 1.327 mmol) and (S)-2-amino-N-(cyclopropyl sulfonyl)-3-((S)-2-oxo pyrrolidin-3-yl) propanamide hydrochloride (amine fragment-29) (165 mg, 0.530 mmol) at 0° C. simultaneously and stirred at room temperature for 16 h. Reaction mixture was diluted with ice water (20 mL) and extracted with ethyl acetate (2×30 mL), dried over sodium sulfate and evaporated under reduced pressure to get crude compound. The crude compound was purified by prep HPLC to afford 3-chlorobenzyl ((S)-3-cyclohexyl-1-(((S)-1-(cyclopropanesulfonamido)-1-oxo-3-((S)-2-oxopyrrolidin-3-yl) propan-2-yl) amino)-1-oxopropan-2-yl) carbamate (Compound C221). TLC system: 15% Methanol in dichloromethane R_{f}: 0.3 LCMS (ESI): m/z 597.2 [M+H]⁺

### Example 169: Synthesis of Compounds C290 and C229

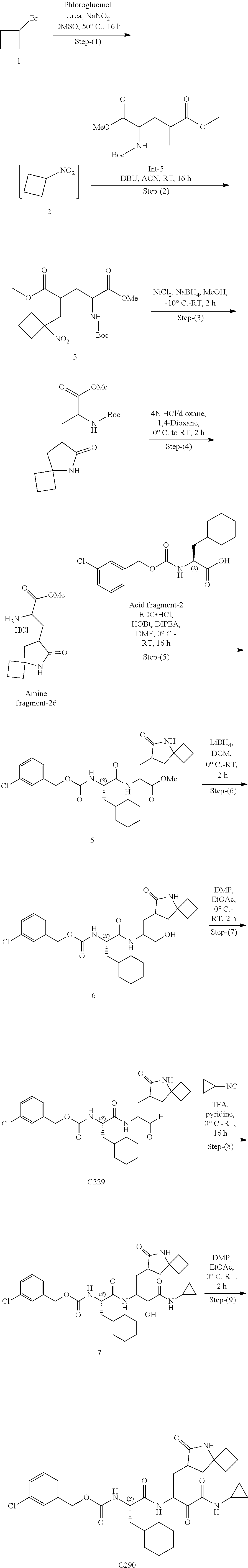

### Nitrocyclobutane (2)

To a stirred solution of bromocyclobutane (1) (10 g, 74.07 mmol) in DMSO (100 mL) was added phloroglucinol (9.3 g, 74.07 mmol), followed by urea (8.88 g, 148.14 mmol) sodium nitrite (25.5 g, 370.37 mmol) at room temperature and the resulting reaction mixture was stirred at 50° C. for 16 h. The progress of the reaction was monitored by TLC. The reaction mixture was quenched with ice cold water (150 mL) and extracted with diethyl ether (3×100 mL), combined organic layers were washed with water (150 ml), brine solution (150 mL), dried over sodium sulfate and evaporated under minimum reduced pressure to afford 2 which was used directly in the next step.

### Dimethyl 2-((tert-butoxycarbonyl)amino)-4-((1-nitrocyclobutyl)methyl)pentanedioate (3)

To a stirred solution of above crude liquid in ACN (60 mL) was added dimethyl 2-((tert-butoxycarbonyl)amino)-4-methylenepentanedioate (Int-5) (5.5 g, 19.8 mmol) and DBU (9 mL, 59.4 mmol) at room temperature and stirring was continued for 2 h. The progress of the reaction was monitored by TLC and LCMS. Excess of ACN was evaporated under reduced pressure. The crude residue was taken in ethyl acetate (200 mL) and washed with water (150 mL) and brine solution (150 mL), dried over sodium sulfate, evaporated and the crude residue was purified by normal phase chromatography to afford dimethyl 2-((tert-butoxycarbonyl)amino)-4-((1-nitrocyclobutyl)methyl)pentanedioate (3). TLC system: 20% Ethyl acetate/Pet ether R_{f}: 0.2 LCMS (ESI): m/z 411.31 [M+Na]⁺

### Methyl 2-((tert-butoxycarbonyl)amino)-3-(6-oxo-5-azaspiro[3.4]octan-7-yl)propanoate (4)

To a stirred solution of dimethyl 2-((tert-butoxycarbonyl)amino)-4-((1-nitrocyclobutyl)methyl)pentanedioate (3) (5 g, 12.8 mmol) in methanol (50 mL) was added nickel chloride (1.67 g, 12.8 mmol), followed by sodium borohydride (2.4 g, 64.4 mmol) at −10° C. and slowly allowed to room temperature and stirred for 2 h. The progress of the reaction was monitored by TLC and LCMS. The reaction mixture was quenched with Sat. ammonium chloride solution (100 mL) and extracted with DCM (3×100 mL), combined organic layers were washed with water (250 mL), followed by brine solution (200 mL), dried over sodium sulfate and evaporated under reduced pressure to get the crude compound and the crude residue was purified by normal phase chromatography to afford methyl 2-((tert-butoxycarbonyl)amino)-3-(6-oxo-5-azaspiro[3.4]octan-7-yl)propanoate (4). TLC system: 10% MeOH/DCM R_{f}: 0.6 LCMS (ESI): m/z 349.25 [M+Na]⁺

### Methyl 2-amino-3-(6-oxo-5-azaspiro[3.4]octan-7-yl)propanoate hydrochloride (Amine fragment-26)

To a stirred solution of methyl 2-((tert-butoxycarbonyl)amino)-3-(6-oxo-5-azaspiro[3.4]octan-7-yl)propanoate (4) (3 g, 9.2 mmol) in 1,4-dioxane (30 mL) was added 4N dioxane.HCl (30 mL) at 0° C. and the resulting reaction mixture was stirred at room temperature for 2 h. The progress of the reaction was monitored by TLC. Reaction mixture was evaporated under reduced pressure. The crude residue was trituration with n-pentane to afford methyl 2-amino-3-(6-oxo-5-azaspiro[3.4]octan-7-yl)propanoate hydrochloride (Amine fragment-26). TLC system: 10% MeOH/DCM R_{f}: 0.1 LCMS (ESI): m/z 227.26 [M+H]⁺

### Methyl 2-((S)-2-((((3-chlorobenzyl)oxy)carbonyl)amino)-3-cyclohexylpropanamido)-3-(6-oxo-5-azaspiro[3.4]octan-7-yl)propanoate (5)

To a stirred solution of (S)-2-((((3-chlorobenzyl)oxy)carbonyl)amino)-3-cyclohexylpropanoic acid (Acid fragment-2) (1.2 g, 3.53 mmol) in DMF (12 mL) was added EDC.HCl (1 g, 5.309 mmol), HOBt (0.716 g, 5.309 mmol), DIPEA (1.88 mL, 10.617 mmol) and methyl 2-amino-3-(6-oxo-5-azaspiro[3.4]octan-7-yl)propanoate hydrochloride (amine fragment-26) (0.927 g, 3.539 mmol) at 0° C. simultaneously and stirred at room temperature for 16 h. Reaction mixture was diluted with ice water (100 mL), extracted with ethyl acetate (2×100 mL), the organic layer was dried over sodium sulphate and evaporated under reduced pressure to afford crude compound. The crude residue was purified by normal phase chromatography to afford methyl 2-((S)-2-((((3-chlorobenzyl)oxy)carbonyl)amino)-3-cyclohexylpropanamido)-3-(6-oxo-5-azaspiro[3.4]octan-7-yl)propanoate (5). TLC system: 10% MeOH/DCM Rf: 0.3 LCMS (ESI): m/z=548.40 [M+H]⁺

### 3-Chlorobenzyl ((2S)-3-cyclohexyl-1-((1-hydroxy-3-(6-oxo-5-azaspiro[3.4]octan-7-yl)propan-2-yl)amino)-1-oxopropan-2-yl)carbamate (6)

To a stirred solution of methyl 2-((S)-2-((((3-chlorobenzyl)oxy)carbonyl)amino)-3-cyclohexylpropanamido)-3-(6-oxo-5-azaspiro[3.4]octan-7-yl)propanoate (5) (1.2 g, 2.193 mmol,) in dichloromethane (25 mL) was added 2M LiBH₄ in THF (2.2 mL, 4.387 mmol) was added at 0° C. and stirred for 2 h. The progress of the reaction was monitored by TLC and LCMS. Reaction mixture was quenched with saturated NH₄Cl solution and extracted with dichloromethane (2×50 mL), dried over sodium sulfate, concentrated under reduced pressure to afford 3-chlorobenzyl ((2S)-3-cyclohexyl-1-((1-hydroxy-3-(6-oxo-5-azaspiro[3.4]octan-7-yl)propan-2-yl)amino)-1-oxopropan-2-yl)carbamate (6). TLC system: 10% Methanol in dichloromethane Rf: 0.4 LCMS (ESI): m/z 520.63 [M+H]⁺

### 3-Chlorobenzyl ((2S)-3-cyclohexyl-1-oxo-1-((1-oxo-3-(6-oxo-5-azaspiro[3.4]octan-7-yl)propan-2-yl)amino)propan-2-yl)carbamate (Compound C229)

To a stirred solution of 3-chlorobenzyl ((2S)-3-cyclohexyl-1-((1-hydroxy-3-(6-oxo-5-azaspiro[3.4]octan-7-yl)propan-2-yl)amino)-1-oxopropan-2-yl)carbamate (6) (150 mg, 0.289 mmol) in ethyl acetate (3 mL) was added Dess-Martin periodinane (183 mg, 0.433 mmol) at 0° C. and stirred at room temperature for 16 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by purified by normal phase chromatography to afford 3-chlorobenzyl ((2S)-3-cyclohexyl-1-oxo-1-((1-oxo-3-(6-oxo-5-azaspiro[3.4]octan-7-yl)propan-2-yl)amino)propan-2-yl)carbamate (Compound C229). TLC system: 10% Methanol in DCM R_{f}: 0.6 LCMS (ESI): m/z 518.2 [M+H]⁺

### 3-Chlorobenzyl ((2S)-3-cyclohexyl-1-((4-(cyclopropylamino)-3-hydroxy-4-oxo-1-(6-oxo-5-azaspiro[3.4]octan-7-yl)butan-2-yl)amino)-1-oxopropan-2-yl)carbamate (7)

To a stirred solution of 3-chlorobenzyl ((2S)-3-cyclohexyl-1-oxo-1-((1-oxo-3-(6-oxo-5-azaspiro[3.4]octan-7-yl)propan-2-yl)amino)propan-2-yl)carbamate (Compound C229) (200 mg, 0.386 mmol) was dissolved in DCM (5 mL), then added pyridine (0.124 mL, 1.512 mmol), isocyanocyclopropane (51 mg, 0.7736 mmol) followed by TFA (0.06 mL, 0.7736 mmol) at 0° C. and stirred at room temperature for 16 h. The progress of the reaction was monitored by TLC and LCMS. Reaction mixture was diluted with dichloromethane and washed with 1N HCl (2×30 mL) followed by water (20 mL) and brine (20 mL). Organic layer was dried over anhydrous Na₂SO₄ and evaporated under reduced pressure to afford crude 3-chlorobenzyl ((2S)-3-cyclohexyl-1-((4-(cyclopropylamino)-3-hydroxy-4-oxo-1-(6-oxo-5-azaspiro[3.4]octan-7-yl)butan-2-yl)amino)-1-oxopropan-2-yl)carbamate (7). TLC system: 10% MeOH in DCM Rf: 0.4 LCMS (ESI): m/z 603.39 [M+H]⁺

### 3-Chlorobenzyl ((2S)-3-cyclohexyl-1-((4-(cyclopropylamino)-3,4-dioxo-1-(6-oxo-5-azaspiro[3.4]octan-7-yl)butan-2-yl)amino)-1-oxopropan-2-yl)carbamate (Compound C290)

To a stirred solution of 3-chlorobenzyl ((2S)-3-cyclohexyl-1-((4-(cyclopropylamino)-3-hydroxy-4-oxo-1-(6-oxo-5-azaspiro[3.4]octan-7-yl)butan-2-yl)amino)-1-oxopropan-2-yl)carbamate (7) (170 mg, 0.282 mmol) in ethyl acetate (5 mL) was added Dess-Martin periodinane (239 mg, 0.564 mmol) at 0° C. and stirred at room temperature for 16 h. The progress of the reaction was monitored by TLC and LCMS. Reaction mixture was filter through celite pad and washed with ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford 3-chlorobenzyl((2S)-3-cyclohexyl-1-((4-(cyclopropylamino)-3,4-dioxo-1-(6-oxo-5-azaspiro[3.4] octan-7-yl)butan-2-yl)amino)-1-oxopropan-2-yl)carbamate (Compound C290). TLC system: 10% MeOH in DCM Rf: 0.45 LCMS (ESI): m/z 601.53 [M+H]⁺

### Example 170: Synthesis of Compound C224

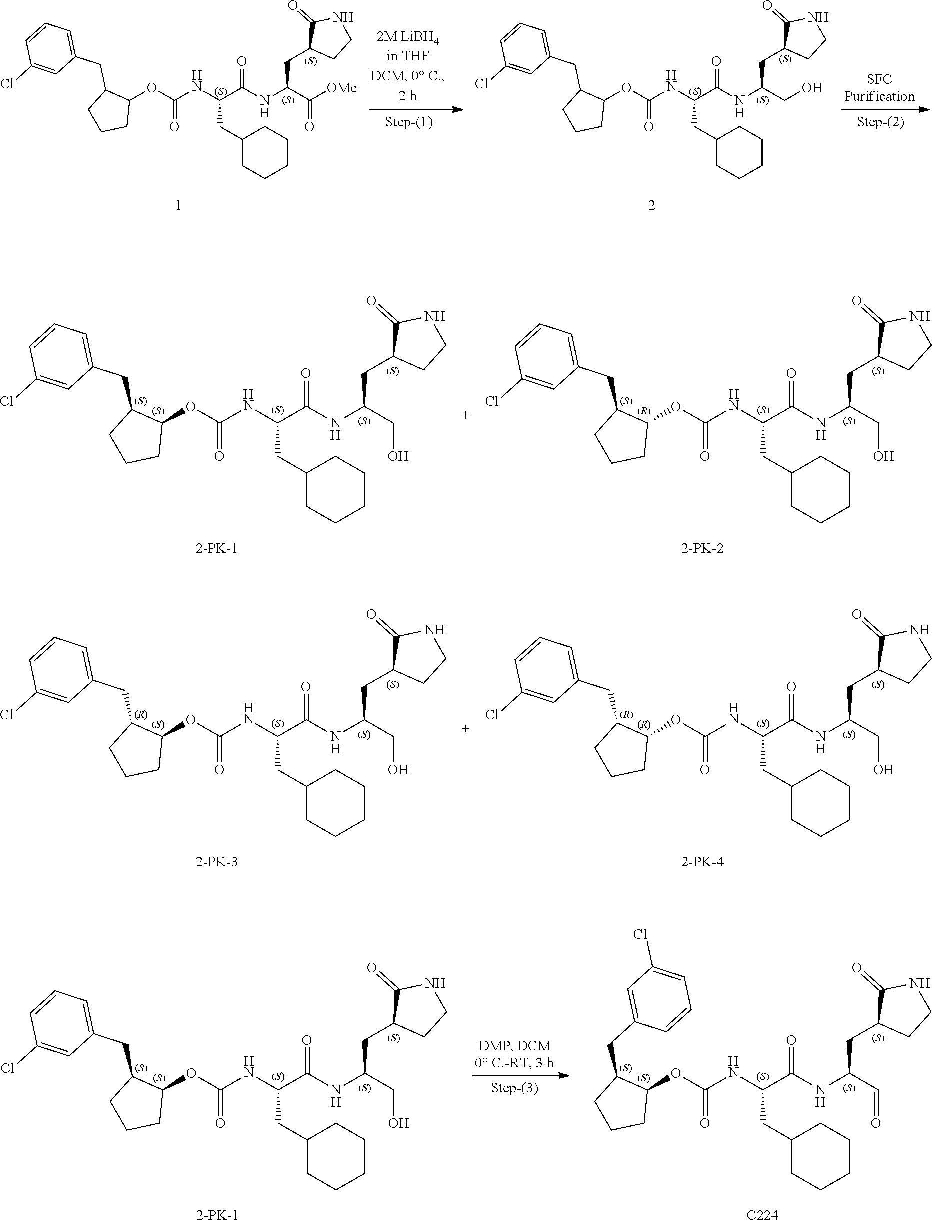

### 2-(3-Chlorobenzyl) cyclopentyl ((S)-3-cyclohexyl-1-(((S)-1-hydroxy-3-((S)-2-oxopyrrolidin-3-yl) propan-2-yl) amino)-1-oxopropan-2-yl) carbamate (2)

To a stirred solution of methyl (2S)-2-((2S)-2-((((2-(3-chlorobenzyl)cyclopentyl)oxy) carbonyl)amino)-3-cyclohexylpropanamido)-3-((S)-2-oxopyrrolidin-3-yl)propanoate (1) (12 g, 20.869 mmol) in dichloromethane (120 mL), was added 2M LiBH₄ in THF (20.8 mL, 41.739 mmol) at 0° C. and stirred for 2 h The progress of the reaction was monitored by TLC and LCMS. Reaction mixture was quenched with saturated NH₄Cl solution and extracted with dichloromethane (2×100 mL), dried over anhydrous Na₂SO₄ and evaporated under reduced pressure to afford 2-(3-chlorobenzyl) cyclopentyl ((S)-3-cyclohexyl-1-(((S)-1-hydroxy-3-((S)-2-oxopyrrolidin-3-yl)propan-2-yl) amino)-1-oxopropan-2-yl) carbamate (2). TLC system: 10% Methanol in dichloromethane Rf: 0.4 LCMS (ESI): m/z=548.57 [M+H]⁺

### Chiral HPLC Purification of 2-(3-chlorobenzyl) cyclopentyl ((S)-3-cyclohexyl-1-(((S)-1-hydroxy-3-((S)-2-oxopyrrolidin-3-yl) propan-2-yl) amino)-1-oxopropan-2-yl) carbamate (2-PK-1, 2-PK-2, 2-PK-3, 2-PK-4)

SFC purification of 2-(3-chlorobenzyl)cyclopentyl ((S)-3-cyclohexyl-1-(((S)-1-hydroxy-3-((S)-2-oxopyrrolidin-3-yl)propan-2-yl)amino)-1-oxopropan-2-yl)carbamate (10 g) by Preparative SFC Conditions Column/dimensions: CHIRALPAK-IA-3 (4.6×250 mm), 3p % CO₂: 60% % Co solvent: 40% (acetonitrile: IPA 1:1), Total Flow: 3.0 g/min Back Pressure: 1500 Psi, Temperature: 30° C. UV: MAX PLOT Solubility: Methanol 2-PK-1, 2-PK-2 2-PK-3 2-PK-4. TLC system: 10% Methanol in dichloromethane Rf: 0.4 LCMS (ESI): m/z=548.57 [M+H]⁺

### (1S,2S)-2-(3-Chlorobenzyl) cyclopentyl ((S)-3-cyclohexyl-1-oxo-1-(((S)-1-oxo-3-((S)-2-oxopyrrolidin-3-yl) propan-2-yl) amino) propan-2-yl) carbamate (Compound C224)

To a stirred solution of (1S,2S)-2-(3-chlorobenzyl)cyclopentyl ((S)-3-cyclohexyl-1-(((S)-1-hydroxy-3-((S)-2-oxopyrrolidin-3-yl)propan-2-yl)amino)-1-oxopropan-2-yl)carbamate (2-PK-1) (0.1 g, 0.182 mmol) was dissolved in dichloromethane (5 mL) was added Dess-Martin periodinane (0.155 g, 0.365 mol) at 0° C. and stirred at room temperature for 3 h. Reaction mixture was diluted with dichloromethane (20 mL) and washed with sat. Hypo solution (3×20 mL), sat. NaHCO₃ solution (3×20 mL). Organic layer was dried over anhydrous Na₂SO₄, filtered and concentrated to afford crude compound. The crude compound was purified by Prep. HPLC to afford (1S,2S)-2-(3-chlorobenzyl)cyclopentyl ((S)-3-cyclohexyl-1-oxo-1-(((S)-1-oxo-3-((S)-2-oxopyrrolidin-3-yl) propan-2-yl)amino)propan-2-yl)carbamate (Compound C224). TLC system: 10% Methanol in dichloromethane Rf: 0.5 LCMS (ESI): m/z=546.2 [M+H]⁺

### Example 171: Synthesis of Compound C225

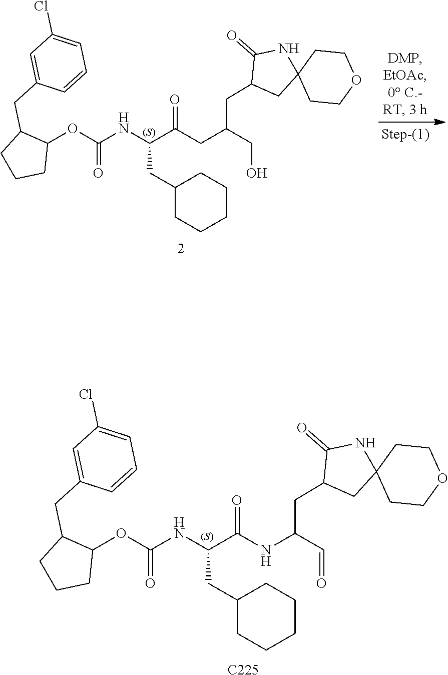

### 2-(3-Chlorobenzyl)cyclopentyl ((2S)-3-cyclohexyl-1-oxo-1-((1-oxo-3-(2-oxo-8-oxa-1-azaspiro[4.5]decan-3-yl)propan-2-yl)amino)propan-2-yl)carbamate (Compound C225)

To a stirred solution of 3-chlorobenzyl 2-(3-chlorobenzyl)cyclopentyl ((2S)-3-cyclohexyl-1-((1-hydroxy-3-(2-oxo-8-oxa-1-azaspiro[4.5]decan-3-yl)propan-2-yl)amino)-1-oxopropan-2-yl)carbamate (1) (400 mg, 0.64 mmol) in ethyl acetate (10 mL) was added Dess-Martin periodinane (823 mg, 1.90 mmol) at 0° C. and stirred at room temperature for 3 h. The progress of the reaction was monitored by TLC and LCMS. Reaction mixture was diluted with ethyl acetate (10 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 2-(3-chlorobenzyl)cyclopentyl ((2S)-3-cyclohexyl-1-oxo-1-((1-oxo-3-(2-oxo-8-oxa-1-azaspiro[4.5]decan-3-yl)propan-2-yl)amino)propan-2-yl)carbamate (Compound C225). TLC system: 5% Methanol in DCM Rt: 0.4 LCMS (ESI): m/z 616.3 (M+H)⁺

### Example 172: Synthesis of Compounds C226 and C246

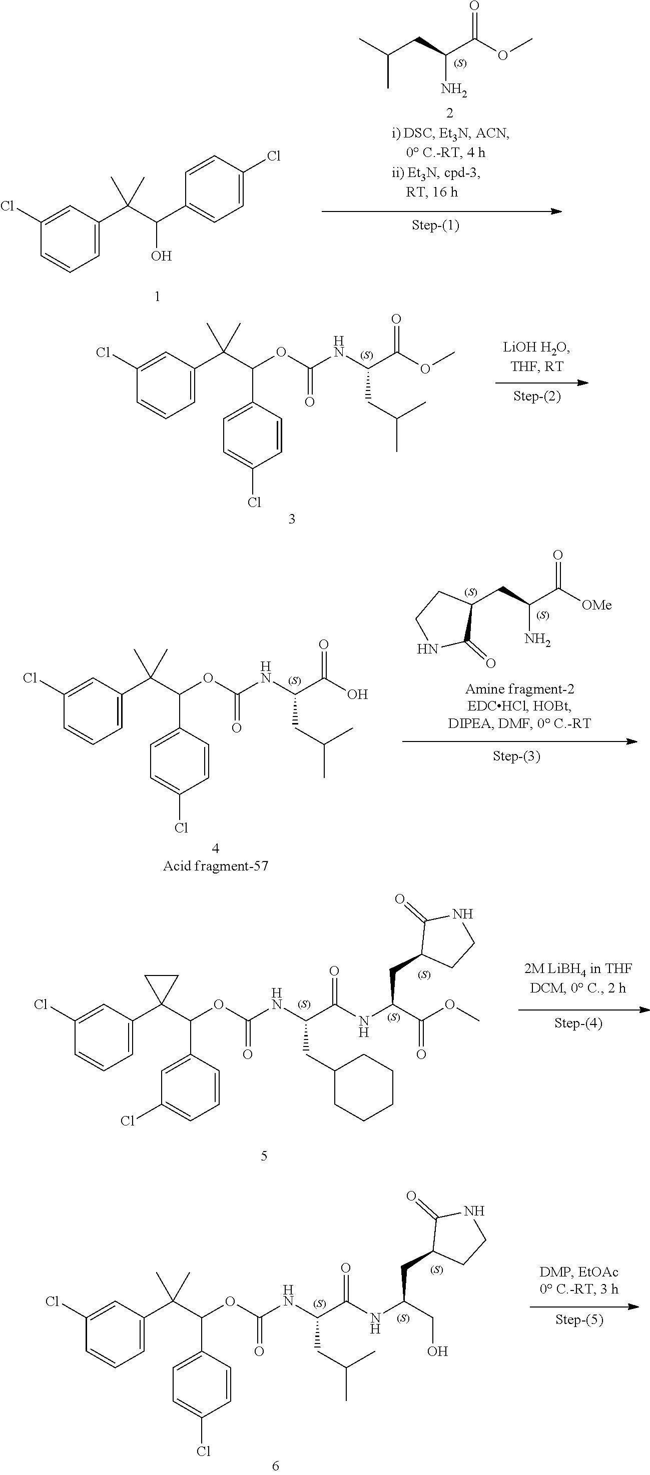

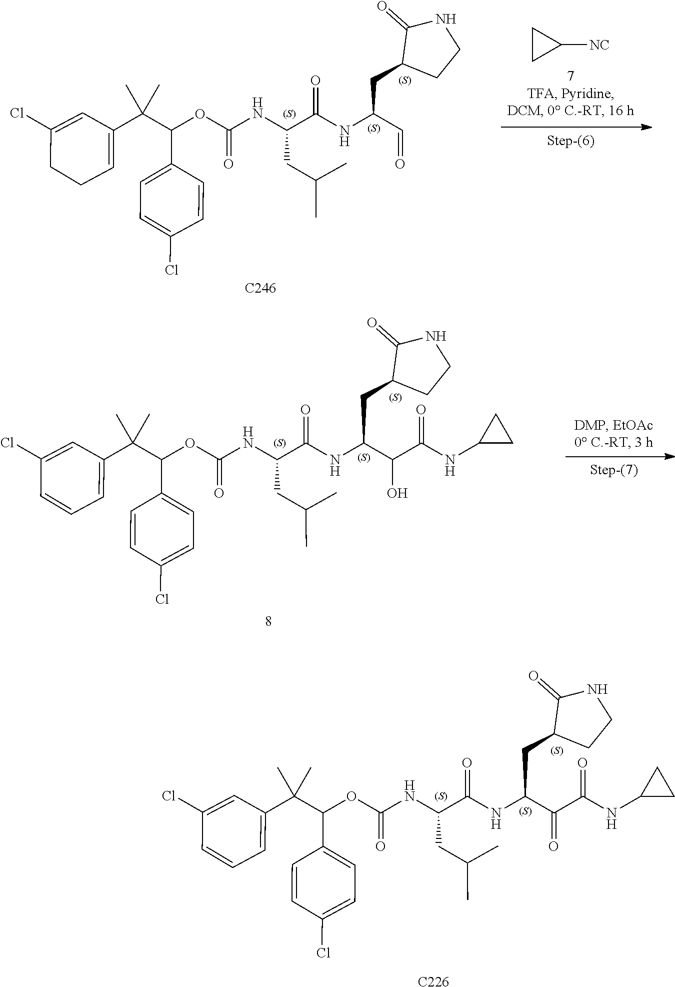

### Methyl ((2-(3-chlorophenyl)-1-(4-chlorophenyl)-2-methylpropoxy)carbonyl)-L-leucinate (1)

To a stirred solution of 2-(3-chlorophenyl)-1-(4-chlorophenyl)-2-methylpropan-1-ol (1) (2.5 g, 8.51 mmol) in acetonitrile (25 mL) ware added Et₃N (4.16 mL, 29.10 mmol), DSC (5.47 g, 21.01 mmol) at 0° C. and stirred at room temperature for 4 h The progress of the reaction was monitored by TLC. Then added methyl L-leucinate (2) (3.08 g, 21.01 mmol) and stirred at room temperature for 16 h. The progress of the reaction was monitored by TLC and LCMS. Reaction mixture was evaporated under reduced pressure and diluted with water (25 mL) and extracted with ethyl acetate (25 mL), organic layer was dried over sodium sulfate, filtered and evaporated under reduced pressure. The crude residue was purified by combi-flash, compound eluted at 10% ethyl acetate in pet ether to afford methyl ((2-(3-chlorophenyl)-1-(4-chlorophenyl)-2-methylpropoxy)carbonyl)-L-leucinate (1). TLC system: 10% Ethyl acetate in hexane Rf: 0.4 LCMS (ESI): m/z=488.23 [M+Na+H]⁺

### ((2-(3-chlorophenyl)-1-(4-chlorophenyl)-2-methylpropoxy)carbonyl)-L-leucine (Acid-fragment-57)

To a stirred solution of methyl ((2-(3-chlorophenyl)-1-(4-chlorophenyl)-2-methylpropoxy)carbonyl)-L-leucinate (1) (1.8 g, 3.80 mmol) in THF (11 mL), water (7 mL) was added lithium hydroxide (325 mg, 7.74 mmol) at 0° C. and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×30 mL), dried over sodium sulfate, concentrated under reduced pressure to ((2-(3-chlorophenyl)-1-(4-chlorophenyl)-2-methylpropoxy)carbonyl)-L-leucine (4). TLC system: 50% Ethyl acetate in hexane Rf: 0.2 LCMS (ESI): m/z 474.2 [M+Na+H]⁺

### Methyl (2S)-2-((2S)-2-((((3-chlorophenyl)(1-(3-chlorophenyl)cyclopropyl) methoxy)carbonyl)amino)-3-cyclohexylpropanamido)-3-((S)-2-oxopyrrolidin-3-yl)propanoate (5)

To a stirred solution of ((2-(3-chlorophenyl)-1-(4-chlorophenyl)-2-methylpropoxy)carbonyl)-L-leucine (4) (1 g, 2.0 mmol) DMF (10 mL) added EDC.HCl (633 mg, 3.31 mmol), HOBt (448 mg, 3.31 mmol), DIPEA (1.1 mL, 6.63 mmol) and methyl (S)-2-amino-3-((S)-2-oxopyrrolidin-3-yl)propanoate (Amine fragment-2) (586 mg, 2.6 mmol) at 0° C. simultaneously and stirred at room temperature for 16 h. The progress of the reaction was monitored by TLC and LCMS. After 16 h, the reaction mixture was diluted with ice water (20 mL), extracted with ethyl acetate (2×30 mL). The combined organic layer was dried over sodium sulfate, filtered and evaporated under reduced pressure. The crude residue was purified by combi-flash compound eluted at 60% Ethyl acetate in pet ether to afford methyl(2S)-2-((2S)-2-((((3-chlorophenyl)(1-(3-chlorophenyl)cyclopropyl) methoxy) carbonyl) amino)-3-cyclohexylpropanamido)-3-((S)-2-oxopyrrolidin-3-yl)propanoate (5). TLC system: Ethyl acetate Rf: 0.5 LCMS (ESI): m/z 620.6 [M+H]⁺

### (2-(3-chlorophenyl)-1-(4-chlorophenyl)-2-methylpropyl ((S)-1-(((S)-1-hydroxy-3-((S)-2-oxopyrrolidin-3-yl)propan-2-yl)amino)-4-methyl-1-oxopentan-2-yl)carbamate (6)

To a stirred solution of methyl (2S)-2-((2S)-2-((((3-chlorophenyl)(1-(3-chlorophenyl)cyclopropyl)methoxy)carbonyl)amino)-3-cyclohexylpropanamido)-3-((S)-2-oxopyrrolidin-3-yl)propanoate (5) (300 mg, 4.8 mmol) in THF (5 mL) was added 2M LiBH4 in THF (0.48 mL, 0.96 mmol) at 0° C. and the reaction mixture stirred for 2 h at 0° C. The progress of the reaction was monitored by TLC and LCMS. Then reaction mixture was quenched with aq. NH4Cl (50 mL) and extracted with ethyl acetate (2×15 mL). Organic layer was washed with brine solution (2×15 mL), dried over Na2SO4 and concentrated to afford 2-(3-chlorophenyl)-1-(4-chlorophenyl)-2-methylpropyl ((S)-1-(((S)-1-hydroxy-3-((S)-2-oxopyrrolidin-3-yl)propan-2-yl)amino)-4-methyl-1-oxopentan-2-yl)carbamate (6). TLC system: Ethyl acetate Rf: 0.2 LCMS (ESI): m/z 592.5 (M+H)⁺

### 2-(5-chlorocyclohexa-1,5-dien-1-yl)-1-(4-chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C246)

To a stirred solution of 2-(3-chlorophenyl)-1-(4-chlorophenyl)-2-methylpropyl ((S)-1-(((S)-1-hydroxy-3-((S)-2-oxopyrrolidin-3-yl)propan-2-yl)amino)-4-methyl-1-oxopentan-2-yl)carbamate (6) (100 mg, 0.130 mmol) was dissolved in ethyl acetate (5 mL) was added Dess-Martin periodinane (172 mg, 0.401 mmol) at 0° C. and stirred at room temperature for 3 h. The progress of the reaction was monitored by TLC and LCMS. Reaction mixture was diluted with ethyl acetate (15 mL) and washed with sat. Hypo solution (3×15 mL), sat. NaHCO₃ solution (3×15 mL). Organic layer was dried over anhydrous Na2SO4, filtered and concentrated to afford 2-(5-chlorocyclohexa-1,5-dien-1-yl)-1-(4-chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C246). TLC system: Ethyl acetate Rf: 0.3 LCMS (ESI): m/z 590.2 (M+H)⁺

### 2-(3-chlorophenyl)-1-(4-chlorophenyl)-2-methylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (8)

To a stirred solution of 2-(5-chlorocyclohexa-1,5-dien-1-yl)-1-(4-chlorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C246) (200 mg, 0.33 mmol) in DCM (10 mL) was added isocyanocyclopropane (7) (45 mg, 0.67 mmol), pyridine (0.10 mL, 1.35 mmol) at 0° C. and the reaction mixture was stirred for 30 minutes at 0° C. and added TFA (0.055 mL, 0.67 mmol) and stirred at room temperature for 16 h. The progress of the reaction was monitored by TLC and LCMS. Reaction mixture was acidified with 1N HCl solution (5 mL) and extracted with DCM (2×10 mL). Organic layer was washed with brine solution (5 mL), dried over Na2SO4 and concentrated to get crude compound. The crude compound to afford 2-(3-chlorophenyl)-1-(4-chlorophenyl)-2-methylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (8). TLC system: 5% Methanol in dichloromethane Rf: 0.2 LCMS (ESI): m/z 675.2 [M+H]⁺

### 2-(3-chlorophenyl)-1-(4-chlorophenyl)-2-methylpropyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C226)

To a stirred solution of 2-(3-chlorophenyl)-1-(4-chlorophenyl)-2-methylpropyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (8) (200 mg, 0.29 mmol) was dissolved in Ethyl acetate (5 mL) was added Dess-Martin periodinane (314 mg, 0.74 mmol) at 0° C. and stirred at room temperature for 3 h. The progress of the reaction was monitored by TLC and LCMS. Reaction mixture was diluted with Ethyl acetate (15 mL) and washed with sat. Hypo solution (3×20 mL), sat. NaHCO₃ solution (3×20 mL). Organic layer was dried over anhydrous Na2SO4, filtered and concentrated to afford crude, this crude was purified by normal phase purification afforded 2-(3-chlorophenyl)-1-(4-chlorophenyl)-2-methylpropyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C226). TLC system: 5% Methanol in DCM Rf: 0.3 LCMS (ESI): m/z 673.3 (M+H)⁺

### Example 173: Synthesis of Compounds C227 and C233

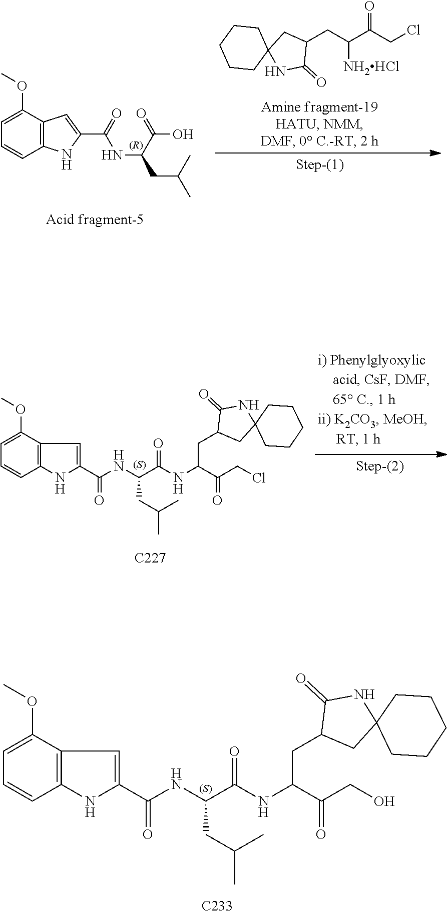

### N-((2S)-1-((4-chloro-3-oxo-1-(2-oxo-1-azaspiro[4.5]decan-3-yl)butan-2-yl)amino)-4-methyl-1-oxopentan-2-yl)-4-methoxy-1H-indole-2-carboxamide (Compound C227)

To a stirred solution of (4-methoxy-1H-indole-2-carbonyl)-D-leucine (Acid fragment-5) (600 mg, 1.973 mmol) and 3-(2-amino-4-chloro-3-oxobutyl)-1-azaspiro[4.5]decan-2-one hydrochloride (Amine fragment-19) (731.8 mg, 2.368 mmol) and in DMF (20 mL) was added HATU (1.12 g, 2.959 mmol) and DIPEA (1.1 mL, 5.921 mmol) at 0° C. simultaneously and stirred at room temperature for 2 h. Reaction mixture was diluted with ice water (50 mL), extracted with ethyl acetate (2×20 mL), the organic layer was dried over sodium sulfate and evaporated under reduced pressure to afford crude compound. The crude residue was purified by silica gel column by eluting with 10% methanol and dichloromethane to afford N-((2S)-1-((4-chloro-3-oxo-1-(2-oxo-1-azaspiro[4.5]decan-3-yl)butan-2-yl)amino)-4-methyl-1-oxopentan-2-yl)-4-methoxy-1H-indole-2-carboxamide (Compound C227). TLC system: 10% MeOH in DCM Rf: 0.6 LCMS (ESI): m/z 559.2 [M+H]⁺

### N-((2S)-1-((4-hydroxy-3-oxo-1-(2-oxo-1-azaspiro[4.5]decan-3-yl)butan-2-yl)amino)-4-methyl-1-oxopentan-2-yl)-4-methoxy-1H-indole-2-carboxamide (Compound C233)

To a stirred solution of N-((2S)-1-((4-chloro-3-oxo-1-(2-oxo-1-azaspiro[4.5]decan-3-yl)butan-2-yl)amino)-4-methyl-1-oxopentan-2-yl)-4-methoxy-1H-indole-2-carboxamide (Compound C227) (540 mg, 0.9667 mmol) Phenyl glyoxylic acid (217.93 mg, 1.45 mmol) in DMF (20 mL) was placed under an atmosphere of N2. This clear pale-yellow solution was treated with cesium fluoride (292.24 mg, 1.93 mmol) followed by heating to 65° C. After 4 hr, the now yellow suspension was cooled to room temperature, diluted with ethyl acetate (60 mL), washed three times water (30 mL), once with brine (30 mL), dried over MgSO4, filtered, and concentrated to give crude 3-((S)-2-(4-methoxy-1H-indole-2-carboxamido)-4-methylpentanamido)-2-oxo-4-(2-oxo-1-azaspiro[4.5]decan-3-yl)butyl 2-oxo-2-phenylacetate as a crude yellow foam. MS (ESI+) for C37H44N4O8 m/z 673.7 (M+H)⁺. This crude was taken in methanol (40 mL) was placed under an atmosphere of N2 and treated with cesium carbonate (144.64 mg, 0.446 mmol) with vigorous stirring. After 1 hr the volatiles were removed in vacuo (bath <30° C.) to give a crude. The crude compound was submitted to Prep HPLC afford N-((2S)-1-((4-hydroxy-3-oxo-1-(2-oxo-1-azaspiro[4.5]decan-3-yl)butan-2-yl)amino)-4-methyl-1-oxopentan-2-yl)-4-methoxy-1H-indole-2-carboxamide (Compound C233) TLC system: 10% MeOH in DCM R_{f}: 0.5 LCMS (ESI): m/z 541.3 [M+H]⁺

### Example 174: Synthesis of Compound C228

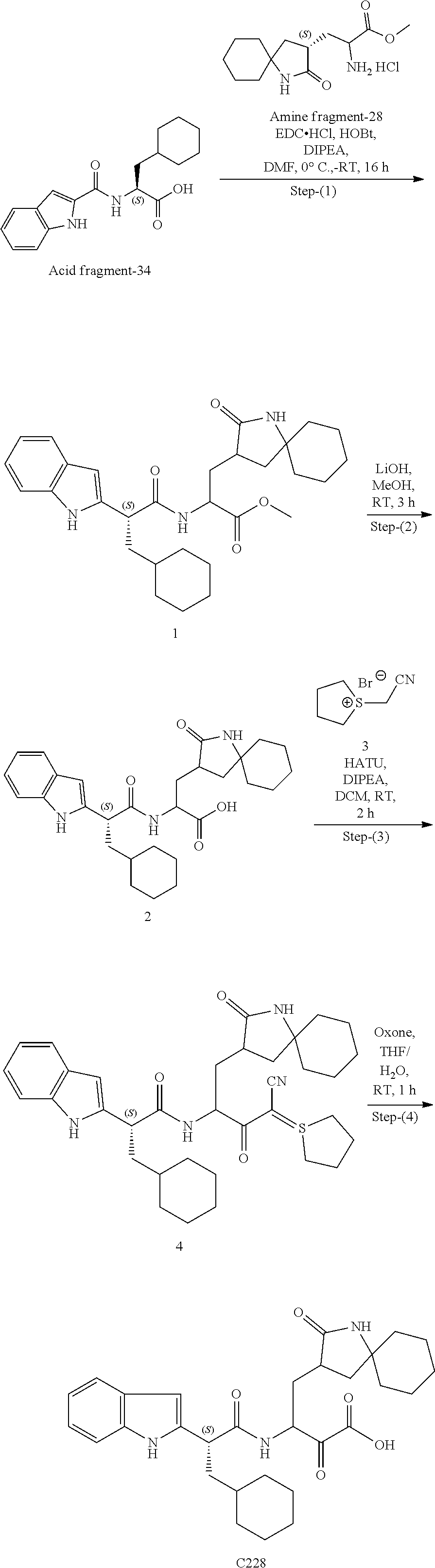

### 2-((S)-3-Cyclohexyl-2-(1H-indole-2-carboxamido)propanamido)-3-(2-oxo-1-azaspiro[4.5]decan-3-yl)propanoate (1)

To a stirred solution of (S)-3-cyclohexyl-2-(1H-indole-2-carboxamido)propanoic acid (Acid fragment-34) (1 g, 3.183 mmol) in DMF (20 mL) was added EDC.HCl (0.91 g, 4.774 mmol), HOBt (0.645 g, 4.774 mmol), DIPEA (2 mL, 9.549 mmol) and methyl (S)-2-amino-3-((S)-2-oxopyrrolidin-3-yl)propanoate (amine fragment-19) (1.2 g, 3.819 mmol) at 0° C. simultaneously and stirred at room temperature for 16 h. Reaction mixture was diluted with ice water (100 mL), extracted with ethyl acetate (2×50 mL), the organic layer was dried over sodium sulfate and evaporated under reduced pressure to afford crude compound. The crude residue was purified by silica gel column by eluting with 50% ethyl acetate and pet-ether to afford methyl 2-((S)-3-cyclohexyl-2-(1H-indole-2-carboxamido)propanamido)-3-(2-oxo-1-azaspiro[4.5]decan-3-yl)propanoate (1). TLC system: 10% Methanol in dichloromethane Rf: 0.3 LCMS (ESI): m/z=537.48 [M+H]⁺

### 2-((S)-3-cyclohexyl-2-(1H-indole-2-carboxamido)propanamido)-3-(2-oxo-1-azaspiro[4.5]decan-3-yl)propanoic acid

To a stirred solution of methyl 2-((S)-3-cyclohexyl-2-(1H-indole-2-carboxamido)propanamido)-3-(2-oxo-1-azaspiro[4.5]decan-3-yl)propanoate (1) (1.5 mg, 2.723 mmol) in THF (10 mL), water (5 mL) was added lithium hydroxide (350 mg, 8.171 mmol) at room temperature and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford 2-((S)-3-cyclohexyl-2-(1H-indole-2-carboxamido)propanamido)-3-(2-oxo-1-azaspiro[4.5]decan-3-yl)propanoic acid (2). TLC system: 15% Methanol in dichloromethane Rf: 0.2 LCMS (ESI): m/z 315.23 [M+H]⁺

### N-((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)-1H-indole-2-carboxamide (4)

To a stirred solution of 2-((S)-3-cyclohexyl-2-(1H-indole-2-carboxamido)propanamido)-3-(2-oxo-1-azaspiro[4.5]decan-3-yl)propanoic acid (2) (1.3 g, 2.425 mmol) DCM (10 mL) added HATU (1.4 g, 3.638 mmol), DIPEA (1.3 mL, 7.276 mmol) and 1-(cyan methyl)tetra hydro-1H-thiophen-1-iumbromide (3) (602 mg, 2.910 mmol) at 0° C. simultaneously and stirred at room temperature for 2 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford N-((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)-1H-indole-2-carboxamide (4). TLC system: 10% Methanol in dichloromethane Rf: 0.4 LCMS (ESI): m/z 646.47 (M+H)⁺

### 3-((S)-3-Cyclohexyl-2-(1H-indole-2-carboxamido)propanamido)-2-oxo-4-(2-oxo-1-azaspiro[4.5]decan-3-yl)butanoic acid (Compound C228)

To a stirred solution of N-((2S)-1-((4-cyano-3-oxo-1-(2-oxo-1-azaspiro[4.5]decan-3-yl)-4-(tetrahydro-1l4-thiophen-1-ylidene)butan-2-yl)amino)-3-cyclohexyl-1-oxopropan-2-yl)-1H-indole-2-carboxamide (4) (500 mg, 0.77 mmol) in THF (10 mL), water (5 mL) was added Oxone (476 mg, 1.54 mmol) at 0° C. and stirred at room temperature for 6 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure. The crude compound was purified by prep HPLC to afford 3-((S)-3-cyclohexyl-2-(1H-indole-2-carboxamido)propanamido)-2-oxo-4-(2-oxo-1-azaspiro[4.5]decan-3-yl)butanoic acid (Compound C228). TLC system: 15% Methanol in dichloromethane Rf: 0.2 LCMS (ESI): m/z 565.2 [M+H]⁺

### Example 175: Synthesis of Compound C230

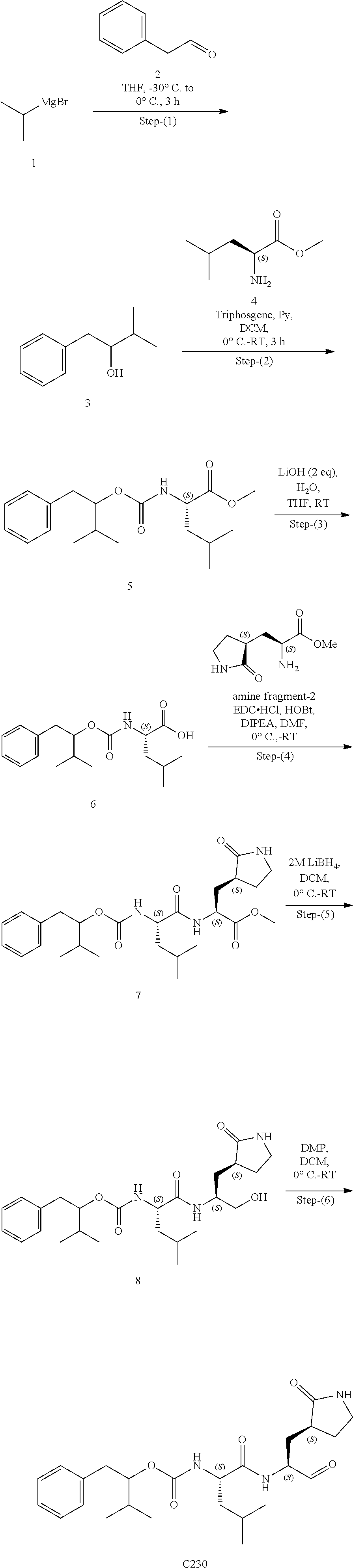

### 3-Methyl-1-phenylbutan-2-ol (3)

To a stirred solution of 2-phenylacetaldehyde (166.6 mL, 333.3 mmol) in THF (400 mL) was added isopropylmagnesium bromide (1) (8 g, 111.1 mmol) at −30° C. and the reaction mixture was stirred at 0° C. for 3 h. The progress of the reaction was monitored by TLC. The reaction mixture was quenched with saturated NH₄Cl (500 mL) and filtered through celite pad and washed with ethyl acetate (2×200 mL), dried over sodium sulfate and evaporated under reduced pressure. The crude residue was purified by silica gel column by eluting with 20% ethyl acetate in hexane to afford 3-Methyl-1-phenylbutan-2-ol (3). TLC system: 20% Ethyl acetate in Hexane Rf: 0.2 LCMS (ESI): m/z=147.1 [M−OH]

### Methyl (((3-methyl-1-phenylbutan-2-yl)oxy)carbonyl)-L-leucinate (5)

To a stirred solution of 3-Methyl-1-phenylbutan-2-ol (3) (6.5 g, 39.5 mmol) in DCM (300 mL) was added pyridine (19.5 mL) methyl L-leucinate hydrochloride (4) (6.8 g, 47.4 mmol) followed by triphosgene (5.84 g, 19.75 mmol) at 0° C. slowly and stirred at room temperature for 16 h. The progress of the reaction was monitored by TLC. Reaction mixture was quenched with 1N aq. HCl (100 mL) then extracted with DCM (2×50 mL), washed with (2×50 mL) NaHCO₃ solution the organic layer was dried over sodium sulfate and evaporated under reduced pressure. The crude residue was purified by silica gel column by eluting with 10% ethyl acetate in hexane to afford Methyl (((3-methyl-1-phenylbutan-2-yl)oxy)carbonyl)-L-leucinate (5). TLC system: 10% Ethyl acetate in hexane Rf: 0.4 LCMS (ESI): m/z=358.29 [M+Na]+

### (((3-Methyl-1-phenylbutan-2-yl)oxy)carbonyl)-L-leucine (6)

To a stirred solution of Methyl (((3-methyl-1-phenylbutan-2-yl)oxy)carbonyl)-L-leucinate (5) (5.0 g, 14.9 mmol) in THF (10 mL), water (10 mL) was added lithium hydroxide (1.83, 44.7 mmol) at room temperature and stirred at room temperature for 2 h. The progress of the reaction was monitored by TLC and LCMS. The reaction mixture was completely distilled under reduced pressure, crude compound acidified with aq. 1N HCl solution up to pH˜2 and extracted with ethyl acetate (2×30 mL), dried over sodium sulfate, concentrated under reduced pressure to afford (((3-Methyl-1-phenylbutan-2-yl)oxy)carbonyl)-L-leucine (6). TLC system: 100% EtOAc Rf: 0.1 LCMS (ESI): m/z 344.39 [M+Na]⁺

### Methyl (2S)-2-((2S)-4-methyl-2-((((3-methyl-1-phenylbutan-2-yl)oxy)carbonyl)amino)pentanamido)-3-((S)-2-oxopyrrolidin-3-yl)propanoate (7)

To a stirred solution of (((3-Methyl-1-phenylbutan-2-yl)oxy)carbonyl)-L-leucine (6) (1 g, 3.11 mmol) in DMF (20 mL) was added EDC.HCl (0.89 g, 4.66 mmol), HOBt (0.629 g, 4.66 mmol), DIPEA (1.71 mL, 9.33 mmol) and methyl (S)-2-amino-3-((S)-2-oxopyrrolidin-3-yl)propanoate (amine fragment-2) (0.901 g, 3.7 mmol) at 0° C. simultaneously and stirred at room temperature for 16 h. Reaction mixture was diluted with ice water (100 mL), extracted with ethyl acetate (2×50 mL), the organic layer was dried over sodium sulfate and evaporated under reduced pressure to afford crude compound. The crude residue was purified by silica gel column by eluting with 10% methanol and dichloromethane to afford methyl (2S)-2-((2S)-4-methyl-2-((((3-methyl-1-phenylbutan-2-yl)oxy)carbonyl)amino)pentanamido)-3-((S)-2-oxopyrrolidin-3-yl)propanoate (7). TLC system: 10% Methanol in dichloromethane Rf: 0.6 LCMS (ESI): m/z=490.85 [M+H]⁺

### 3-Methyl-1-phenylbutan-2-yl ((S)-1-(((S)-1-hydroxy-3-((S)-2-oxopyrrolidin-3-yl)propan-2-yl)amino)-4-methyl-1-oxopentan-2-yl)carbamate (8)

To a stirred solution of methyl (2S)-2-((2S)-4-methyl-2-((((3-methyl-1-phenylbutan-2-yl)oxy)carbonyl)amino)pentanamido)-3-((S)-2-oxopyrrolidin-3-yl)propanoate (7) (0.8 g, 1.63 mmol) in dichloromethane (30 mL) was added 2M LiBH₄ in THF (3.2 mL, 3.27 mmol) was added at 0° C. and stirred for 2 h. The progress of the reaction was monitored by TLC and LCMS. Reaction mixture was quenched with saturated NH₄Cl solution and extracted with dichloromethane (2×20 mL), dried over sodium sulfate, concentrated under reduced pressure to afford 3-methyl-1-phenylbutan-2-yl ((S)-1-(((S)-1-hydroxy-3-((S)-2-oxopyrrolidin-3-yl)propan-2-yl)amino)-4-methyl-1-oxopentan-2-yl)carbamate (8). TLC system: 10% Methanol in dichloromethane Rf: 0.2 LCMS (ESI): m/z 462.46 [M+H]⁺

### 3-Methyl-1-phenylbutan-2-yl ((S)-4-methyl-1-oxo-1-(((S)-1-oxo-3-((S)-2-oxopyrrolidin-3-yl)propan-2-yl)amino)pentan-2-yl)carbamate (Compound C230)

To a stirred solution of 3-methyl-1-phenylbutan-2-yl ((S)-1-(((S)-1-hydroxy-3-((S)-2-oxopyrrolidin-3-yl)propan-2-yl)amino)-4-methyl-1-oxopentan-2-yl)carbamate (8) (300 mg, 0.644 mmol) was dissolved in ethyl acetate (10 mL) was added Dess-Martin periodinane (551.1 mg, 1.229 mmol) at 0° C. and stirred at room temperature for 3 h. Reaction mixture was diluted with dichloromethane (20 mL) and washed with sat. Hypo solution (3×20 mL), sat. NaHCO₃ solution (3×20 mL) and brine (1×20 mL). Organic layer was dried over anhydrous Na₂SO₄, filtered and concentrated to afford crude compound. The crude compound was submitted to Prep HPLC afford 3-methyl-1-phenylbutan-2-yl ((S)-4-methyl-1-oxo-1-(((S)-1-oxo-3-((S)-2-oxopyrrolidin-3-yl)propan-2-yl)amino)pentan-2-yl)carbamate (Compound C230). Rf: 0.2 LCMS (ESI): m/z 460.2 [M+H]⁺

### Example 176: Synthesis of Compound C231

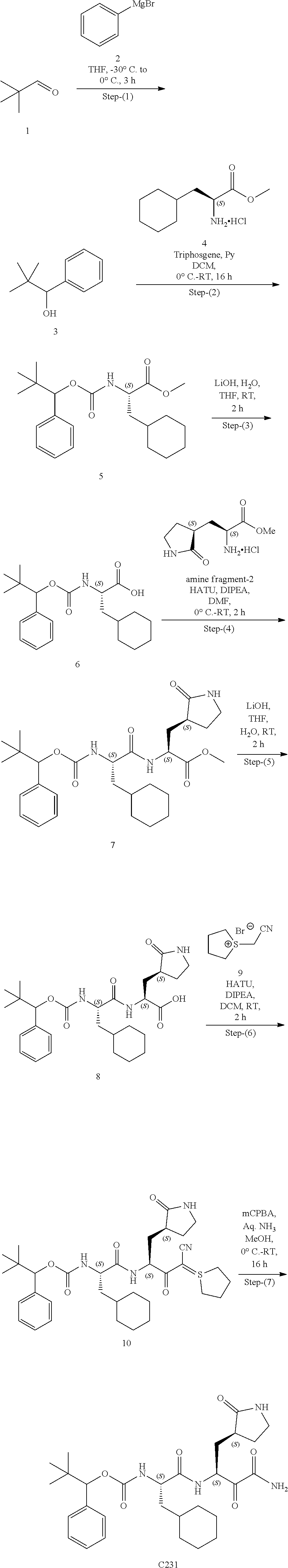

### 2,2-Dimethyl-1-phenylpropan-1-ol (3)

To a stirred solution of phenyl magnesium bromide (2) (116 mL, 116.27 mmol) in THF (100 mL) wad added pivalaldehyde (1) (5 g, 58.13 mmol) at −30° C. and stirred at room temperature for 3 h. Reaction progress was monitored by TLC and LCMS. Reaction mixture was quenched with sat. ammonium chloride and extracted with ethyl acetate (2×50 mL). Combined the organic layer and washed with brine solution (2×50 mL), dried over sodium sulfate and evaporated under reduced pressure. The crude residue was purified by combiflash NP, compound eluted at 8% ethyl acetate in hexane to afford 2,2-dimethyl-1-phenylpropan-1-ol (3). TLC system: 10% Ethyl acetate in hexane Rf: 0.3 LCMS (ESI): m/z 147.06 [M−OH]⁺

### Methyl (2S)-3-cyclohexyl-2-(((2,2-dimethyl-1-phenylpropoxy) carbonyl) amino)propanoate (5)

To a stirred solution of 2,2-dimethyl-1-phenylpropan-1-ol (3) (3 g, 18.29 mmol), methyl (S)-2-amino-3-cyclohexylpropanoate hydrochloride (4) (4.0 g, 21.94 mmol) in dichloromethane (40 mL) was added pyridine (9 mL, 3 vol) followed by triphosgene (2.7 g, 9.14 mmol) at 0° C. and stirred at room temperature for 16 h. The progress of the reaction was monitored by TLC and LCMS. Reaction mixture was diluted with DCM and washed with 1N HCl (50 mL), organic layer was washed with brine solution (50 mL) and dried over sodium sulfate, filtered and evaporated under reduced pressure. The crude residue was purified by combi-flash, compound eluted at 10% ethyl acetate in pet ether to afford methyl (2S)-3-cyclohexyl-2-(((2,2-dimethyl-1-phenylpropoxy) carbonyl) amino) propanoate (5). TLC system: 10% Ethyl acetate in hexane Rf: 0.3 LCMS (ESI): m/z 432.32 [M+H]⁺

### (2S)-3-Cyclohexyl-2-(((2,2-dimethyl-1-phenylpropoxy)carbonyl)amino)propanoic acid (6)

To a stirred solution of methyl (2S)-3-cyclohexyl-2-(((2,2-dimethyl-1-phenylpropoxy) carbonyl)amino)propanoate (5) (6.0 g, 16.00 mmol) in THF (15 mL), water (15 mL) was added lithium hydroxide (1.15 g, 48.00 mmol) at room temperature and stirred at room temperature for 2 h. The progress of the reaction was monitored by TLC and LCMS. The reaction mixture was completely distilled under reduced pressure, crude compound acidified with aq. 1N HCl solution up to pH˜2 and extracted with ethyl acetate (2×50 mL), dried over sodium sulfate, concentrated under reduced pressure to afford (2S)-3-cyclohexyl-2-(((2,2-dimethyl-1-phenylpropoxy)carbonyl)amino)propanoic acid (6). TLC system: 100% EtOAc Rf: 0.1 LCMS (ESI): m/z 362.40 [M+H]⁺

### Methyl (2S)-2-((2S)-3-cyclohexyl-2-(((2,2-dimethyl-1-phenylpropoxy) carbonyl) amino) propanamido)-3-((S)-2-oxopyrrolidin-3-yl) propanoate (7)

To a stirred solution of (2S)-3-cyclohexyl-2-(((2,2-dimethyl-1-phenylpropoxy) carbonyl)amino)propanoic acid (6) (2 g, 5.54 mmol) DMF (20 mL) added HATU (5.2 g, 13.85 mmol), DIPEA (2.6 mL, 16.62 mmol) and methyl (S)-2-amino-3-((S)-2-oxopyrrolidin-3-yl) propanoate hydrochloride (amine fragment-2) (1.23 g, 6.6 mmol) at 0° C. simultaneously and stirred at room temperature for 16 h. Reaction mixture was quenched with ice water (100 mL) and extracted with ethyl acetate (2×50 mL). Combined the organic layer and washed with brine solution (50 mL), dried over sodium sulfate and evaporated under reduced pressure. The crude residue was purified by combi-flash NP, compound eluted at 5% methanol in dichloromethane to afford methyl (methyl (2S)-2-((2S)-3-cyclohexyl-2-(((2,2-dimethyl-1-phenylpropoxy) carbonyl) amino) propanamido)-3-((S)-2-oxopyrrolidin-3-yl) propanoate (7). TLC system: 10% Methanol in dichloromethane Rf: 0.4 LCMS (ESI): m/z 530.70 [M+H]⁺

### ((2S)-2-((2S)-3-cyclohexyl-2-(((2,2-dimethyl-1-phenyl propoxy) carbonyl) amino) propanamido)-3-((S)-2-oxopyrrolidin-3-yl) propanoic acid (8)

To a stirred solution of (methyl (2S)-2-((2S)-3-cyclohexyl-2-(((2,2-dimethyl-1-phenyl propoxy)carbonyl) amino) propanamido)-3-((S)-2-oxopyrrolidin-3-yl) propanoate (7) (700 mg, 1.32 mmol) in THF (10 mL), water (3 mL) was added lithium hydroxide (95 mg, 3.96 mmol) at room temperature and stirred at room temperature for 2 h. The progress of the reaction was monitored by TLC and LCMS. Reaction mixture completely distilled under reduced pressure, crude compound acidified with aq. 1N HCl solution up to pH˜2 and extracted with ethyl acetate (2×30 mL), dried over sodium sulfate, concentrated under reduced pressure to afford ((2S)-2-((2S)-3-cyclohexyl-2-(((2,2-dimethyl-1-phenylpropoxy)carbonyl)amino)propanamido)-3-((S)-2-oxo pyrrolidin-3-yl)propanoic acid (8). TLC system: 100% EtOAc Rf: 0.1 LCMS (ESI): m/z 516.60 [M+H]⁺

### 2,2-Dimethyl-1-phenylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (10)

To a stirred solution of (2S)-2-((2S)-3-cyclohexyl-2-(((2,2-dimethyl-1-phenylpropoxy) carbonyl)amino)propanamido)-3-((S)-2-oxopyrrolidin-3-yl)propanoic acid (8) (500 mg, 0.97 mmol) DCM (10 mL) added HATU (737 mg, 1.94 mmol), DIPEA (0.45 mL, 2.91 mmol) and 1-(cyan methyl)tetra hydro-1H-thiophen-1-iumbromide (9) (500 mg, 2.42 mmol) at 0° C. simultaneously and stirred at room temperature for 2 h. Reaction mixture was diluted with ice water (20 mL), extracted with dichloromethane (2×20 mL), dried over sodium sulfate and evaporated under reduced pressure. The crude residue was purified by combi-flash NP, compound eluted at 3% methanol in dichloromethane to afford 2,2-dimethyl-1-phenylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (10). TLC system: 10% Methanol in dichloromethane Rf: 0.3 LCMS (ESI): m/z 625.91 [M+H]⁺

### 2,2-Dimethyl-1-phenylpropyl ((S)-1-(((S)-4-amino-3,4-dioxo-1-((S)-2-oxopyrrolidin-3-yl) butan-2-yl) amino)-3-cyclohexyl-1-oxopropan-2-yl) carbamate (Compound C231)

To a stirred solution of 2,2-dimethyl-1-phenylpropyl ((S)-1-(((S)-4-cyano-3-oxo-1-((S)-2-oxopyrrolidin-3-yl)-4-(tetrahydro-1l4-thiophen-1-ylidene)butan-2-yl)amino)-3-cyclohexyl-1-oxopropan-2-yl)carbamate (10) (200 mg, 0.32 mmol) in methanol (5 mL) was added m-CPBA (165 mg, 0.96 mmol) at 0° C. and the reaction mixture stirred for 2 h at 0° C. To this was added aq ammonia (2 mL) and stirred at room temperature for 16 h. The progress of the reaction was monitored by TLC and LCMS. Reaction mixture was quenched with sat. NaHCO₃ solution (20 mL) and extracted with DCM (2×20 mL). Organic layer was washed with brine solution (30 mL), dried over Na₂SO₄ and concentrated to get crude compound. The crude compound was purified by prep HPLC to afford 2,2-dimethyl-1-phenylpropyl ((S)-1-(((S)-4-amino-3,4-dioxo-1-((S)-2-oxopyrrolidin-3-yl)butan-2-yl)amino)-3-cyclohexyl-1-oxopropan-2-yl)carbamate (Compound C231). TLC system: 10% Methanol in dichloromethane Rf: 0.4

### Example 177: Synthesis of Compound C232

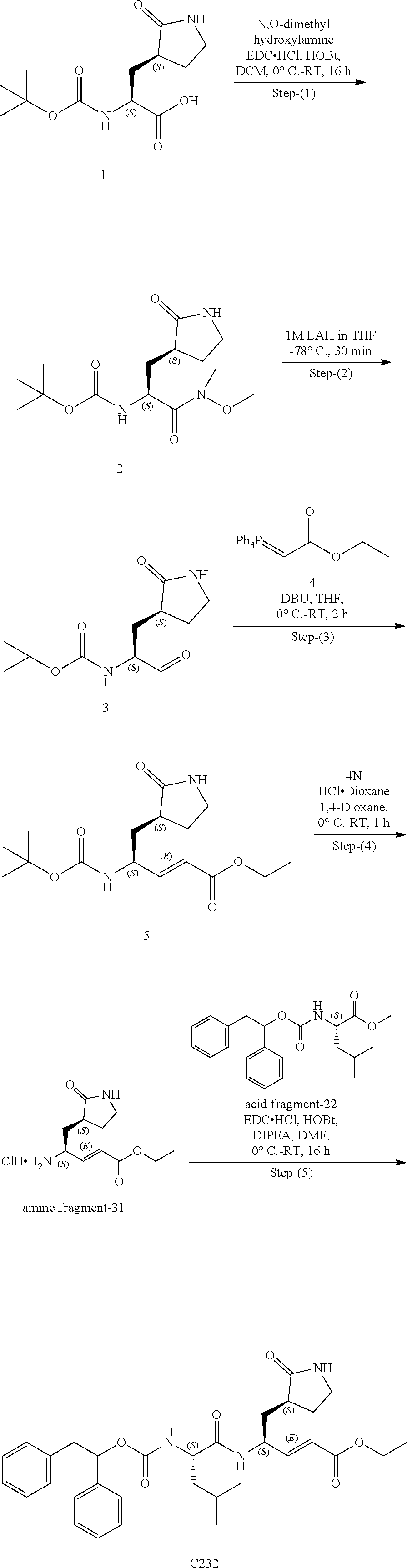

### Tert-butyl ((S)-1-(methoxy (methyl) amino)-1-oxo-3-((S)-2-oxopyrrolidin-3-yl) propan-2-yl) carbamate (2)

To a stirred solution of (S)-2-((tert-butoxycarbonyl)amino)-3-((S)-2-oxopyrrolidin-3-yl)propanoic acid (1) (5 g, 18.38 mmol) in DMF (40 mL) was added EDC.HCl (5.2 g, 27.53 mmol), HOBt (3.72 g, 27.53 mmol), NMM (5.56 mL, 55.14 mmol) and N,O-dimethyl hydroxylamine (2.13 g, 22.058 mmol) at 0° C. simultaneously and stirred at room temperature for 16 h. Reaction mixture was diluted with ice water (100 mL), extracted with ethyl acetate (2×50 mL), the organic layer was dried over sodium sulfate and evaporated under reduced pressure to afford tert-butyl ((S)-1-(methoxy(methyl)amino)-1-oxo-3-((S)-2-oxopyrrolidin-3-yl)propan-2-yl)carbamate (2). TLC system: 10% Methanol in DCM Rf: 0.4 LCMS (ESI): m/z 316.30 [M+H]⁺

### Tert-butyl ((S)-1-oxo-3-((S)-2-oxopyrrolidin-3-yl) propan-2-yl) carbamate (3)

To a stirred solution of tert-butyl ((S)-1-(methoxy (methyl) amino)-1-oxo-3-((S)-2-oxopyrrolidin-3-yl) propan-2-yl) carbamate (2) (1 g, 3.17 mmol) in THF was added 1M LAH (1.32 mL, 3.17 mmol) at −78° C. and stirred for 30 min. The progress of the reaction was monitored by TLC and LCMS. Reaction mixture was quenched with sat ammonium chloride and extracted with ethyl acetate (2×30 mL), the organic layer was dried over sodium sulfate and evaporated under reduced pressure to afford tert-butyl ((S)-1-oxo-3-((S)-2-oxopyrrolidin-3-yl)propan-2-yl)carbamate (3). TLC system: 10% Methanol in DCM R_{f}: 0.3

### Ethyl (S,E)-4-((tert-butoxycarbonyl) amino)-5-((S)-2-oxopyrrolidin-3-yl) pent-2-enoate (5)

To a stirred solution of Tert-butyl ((S)-1-oxo-3-((S)-2-oxopyrrolidin-3-yl)propan-2-yl)carbamate (3) (250 mg, 0.97 mmol), ethyl 2-(triphenyl-l5-phosphaneylidene)acetate (348 mg, 0.976 mmol) in THF (10 mL) as added DBU (148 mg, 0.976 mmol) at room temperature and stirred for 2 h. The progress of the reaction was monitored by TLC. Reaction mixture was quenched with sat ammonium chloride and extracted with ethyl acetate (2×20 mL), the organic layer was dried over sodium sulfate and evaporated under reduced pressure to afford crude compound. The crude residue was purified by silica gel column by eluting with 2% methanol in dichloromethane to afford ethyl (S,E)-4-((tert-butoxycarbonyl)amino)-5-((S)-2-oxopyrrolidin-3-yl)pent-2-enoate (5). TLC system: 10% Methanol in DCM R_{f}: 0.4 LCMS (ESI): m/z 327.21 (M+H)⁺

### Ethyl (S,E)-4-amino-5-((S)-2-oxopyrrolidin-3-yl) pent-2-enoate hydrochloride (amine fragment-31)

To a stirred solution of ethyl (S,E)-4-((tert-butoxycarbonyl)amino)-5-((S)-2-oxopyrrolidin-3-yl)pent-2-enoate (5) (350 mg, 1.073 mmol) in 1,4 dioxane (5 mL) at 0° C. was added drop wise 4N HCl in dioxane (5 mL) and the reaction mixture was stirred at room temperature for 2 h. The progress of the reaction was monitored by TLC. The reaction mixture was evaporated under reduced pressure to obtained crude compound, the resulting crude triturated with diethyl ether to afford ethyl (S,E)-4-amino-5-((S)-2-oxopyrrolidin-3-yl)pent-2-enoate hydrochloride (amine fragment-31). TLC system: 10% Methanol in DCM R_{f}: 0.1 LCMS (ESI): m/z 227.30 (M+H)⁺

### Ethyl (4S, E)-4-((2S)-2-(((1,2-diphenylethoxy) carbonyl) amino)-4-methylpentanamido)-5-((S)-2-oxopyrrolidin-3-yl) pent-2-enoate (Compound C232)

To a stirred solution of ((1,2-diphenylethoxy)carbonyl)-L-leucine (acid fragment-22) (200 mg, 0.563 mmol) in DMF (6 mL) was added EDC.HCl (161 mg, 0.84 mmol), HOBt (114 mg, 0.84 mmol), DIPEA (0.2 mL, 1.68 mmol) and ethyl (S,E)-4-amino-5-((S)-2-oxopyrrolidin-3-yl)pent-2-enoate hydrochloride (amine fragment-31)) (152 mg, 0.675 mmol) at 0° C. simultaneously and stirred at room temperature for 16 h. Reaction mixture was diluted with ice water (20 mL) and extracted with ethyl acetate (2×20 mL), dried over sodium sulfate and evaporated under reduced pressure to get crude compound. The crude compound was purified by prep HPLC to afford ethyl (4S, E)-4-((2S)-2-(((1,2-diphenylethoxy) carbonyl) amino)-4-methylpentanamido)-5-((S)-2-oxopyrrolidin-3-yl) pent-2-enoate (Compound C232). TLC system: 10% Methanol in dichloromethane R_{f}: 0.4 LCMS (ESI): m/z 564.3 [M+H]⁺

### Example 178: Synthesis of Compound C234

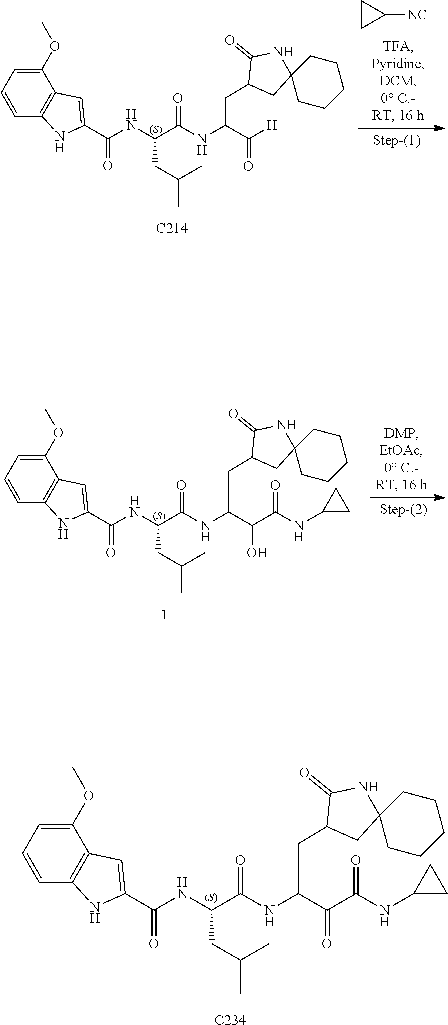

### N-((2S)-1-((4-(cyclopropylamino)-3-hydroxy-4-oxo-1-(2-oxo-1-azaspiro[4.5]decan-3-yl)butan-2-yl)amino)-4-methyl-1-oxopentan-2-yl)-4-methoxy-1H-indole-2-carboxamide (1)

To a stirred solution of methyl 4-methoxy-N-((2S)-4-methyl-1-oxo-1-((1-oxo-3-(2-oxo-1-azaspiro[4.5]decan-3-yl)propan-2-yl)amino)pentan-2-yl)-1H-indole-2-carboxamide (Compound C214) (350 mg, 0.686 mmol) in DCM 5 mL) was added isocyanocyclopropane (69 mg, 1.02 mmol), pyridine (216 mg, 2.74 mmol) then stirred for 15 min, followed by TFA (80 mg, 0.75 mmol) was added at 0° C. and stirred at room temperature for 16 h. The progress of the reaction was monitored by TLC and LCMS. After 16 h, the reaction mixture was diluted with dichloromethane and washed with 1N HCl solution (15 mL) followed by brine (15 mL), dried over sodium sulfate, concentrated under reduce pressure to afford crude residue. This residue was triturated with di ethyl ether (30 mL) to afford N-((2S)-1-((4-(cyclopropylamino)-3-hydroxy-4-oxo-1-(2-oxo-1-azaspiro[4.5]decan-3-yl)butan-2-yl)amino)-4-methyl-1-oxopentan-2-yl)-4-methoxy-1H-indole-2-carboxamide (1). TLC system: 10% Methanol in dichloromethane Rf: 0.4 LCMS (ESI): m/z 596.63 [M+H]⁺

### N-((2S)-1-((4-(cyclopropylamino)-3,4-dioxo-1-(2-oxo-1-azaspiro[4.5]decan-3-yl)butan-2-yl)amino)-4-methyl-1-oxopentan-2-yl)-4-methoxy-1H-indole-2-carboxamide (Compound C234)

To a stirred solution of N-((2S)-1-((4-(cyclopropylamino)-3-hydroxy-4-oxo-1-(2-oxo-1-azaspiro[4.5]decan-3-yl)butan-2-yl)amino)-4-methyl-1-oxopentan-2-yl)-4-methoxy-1H-indole-2-carboxamide (1) (140 mg, 0.23 mmol) was dissolved in ethyl acetate (5 mL) was added Dess-Martin periodinane (149 mg, 0.35 mmol) at 0° C. and stirred at room temperature for 16 h. Reaction mixture was filtered through celite bed and washed with ethyl acetate (20 mL). Filtrate was washed with sat. Hypo solution (3×20 mL) followed by sat. NaHCO₃ solution (3×20 mL). Organic layer was dried over anhydrous Na₂SO₄, filtered and concentrated to get crude compound. The crude residue was purified by Prep-HPLC, to afford N-((2S)-1-((4-(cyclopropylamino)-3,4-dioxo-1-(2-oxo-1-azaspiro[4.5]decan-3-yl)butan-2-yl)amino)-4-methyl-1-oxopentan-2-yl)-4-methoxy-1H-indole-2-carboxamide (Compound C234). TLC system: 10% Methanol in dichloromethane Rf: 0.5 LCMS (ESI): m/z 594.3 [M+H]⁺

### Example 179: Synthesis of Compound C237

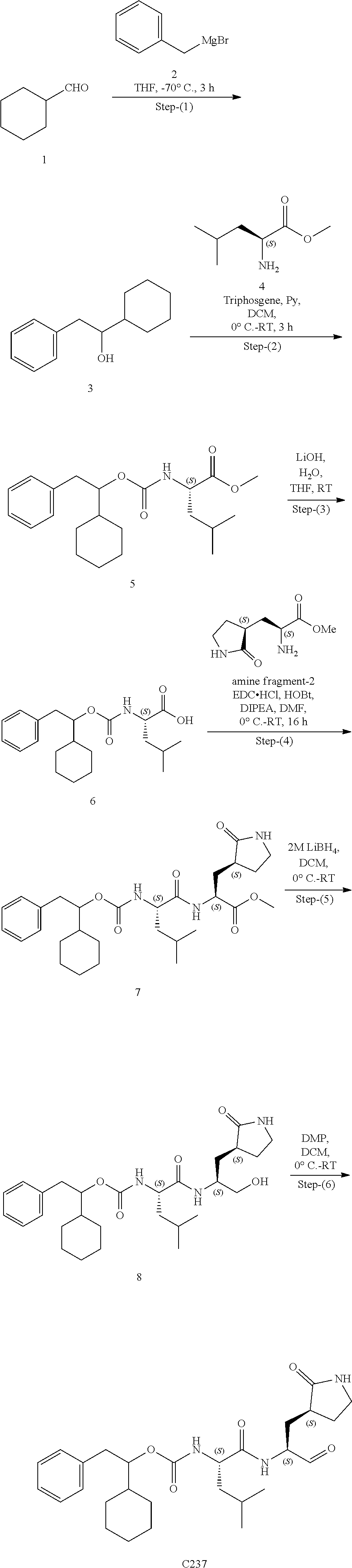

To a stirred solution of cyclohexanecarbaldehyde (1) (10 g, 0.0998 mmol) in THF (100 mL) was added Benzyl magnesium bromide (2) (250 mL, 0.2496 mmol) at −70° C. and the reaction mixture was stirred at 0° C. for 3 h. The progress of the reaction was monitored by TLC. The reaction mixture was quenched with saturated NH₄Cl (500 mL) and filtered through celite pad and washed with ethyl acetate (2×200 mL), dried over sodium sulfate and evaporated under reduced pressure. The crude residue was purified by silica gel column by eluting with 20% ethyl acetate in hexane to afford 1-cyclohexyl-2-phenylethan-1-ol (3). TLC system: 20% Ethyl acetate in Hexane Rf: 0.2 LCMS (ESI): m/z=187.2 [M−OH]

### Methyl ((1-cyclohexyl-2-phenylethoxy)carbonyl)-L-leucinate (5)

To a stirred solution of 1-cyclohexyl-2-phenylethan-1-ol (3) (6 g, 29.3 mmol) in DCM (100 mL) was added pyridine (18 mL) methyl L-leucinate hydrochloride (4) (5.1 g, 35.1 mmol) followed by triphosgene (4.4 g, 14.6 mmol) at 0° C. slowly and stirred at room temperature for 16 h. The progress of the reaction was monitored by TLC. Reaction mixture was quenched with 1N aq. HCl (100 mL) then extracted with DCM (2×50 mL), washed with (2×50 mL) NaHCO₃ solution the organic layer was dried over sodium sulfate and evaporated under reduced pressure. The crude residue was purified by silica gel column by eluting with 10% ethyl acetate in hexane to afford Methyl ((1-cyclohexyl-2-phenylethoxy)carbonyl)-L-leucinate (5). TLC system: 10% Ethyl acetate in hexane Rf: 0.4 LCMS (ESI): m/z=398.35 [M+Na]+

### ((1-cyclohexyl-2-phenylethoxy)carbonyl)-L-leucine (6)

To a stirred solution of methyl ((1-cyclohexyl-2-phenylethoxy)carbonyl)-L-leucinate (5) (2.5 g, 6.663 mmol) in THF (10 mL), water (10 mL) was added lithium hydroxide (600 mg, 13.32 mmol) at room temperature and stirred at room temperature for 2 h. The progress of the reaction was monitored by TLC and LCMS. The reaction mixture was completely distilled under reduced pressure, crude compound acidified with aq. 1N HCl solution up to pH˜2 and extracted with ethyl acetate (2×30 mL), dried over sodium sulfate, concentrated under reduced pressure to afford (((3-Methyl-1-phenylbutan-2-yl)oxy)carbonyl)-L-leucine (6). TLC system: 100% EtOAc Rf: 0.1 LCMS (ESI): m/z 384.47 [M+Na]⁺

### Methyl (2S)-2-((2S)-2-(((1-cyclohexyl-2-phenylethoxy)carbonyl)amino)-4-methylpentanamido)-3-((S)-2-oxopyrrolidin-3-yl)propanoate (7)

To a stirred solution of ((1-cyclohexyl-2-phenylethoxy)carbonyl)-L-leucine (6) (1 g, 2.7683 mmol) in DMF (20 mL) was added EDC.HCl (0.79 g, 4.1524 mmol), HOBt (0.561 g, 4.1524 mmol), DIPEA (1.6 mL, 8.3049 mmol) and methyl (S)-2-amino-3-((S)-2-oxopyrrolidin-3-yl)propanoate (amine fragment-2) (0.620 g, 3.3219 mmol) at 0° C. simultaneously and stirred at room temperature for 16 h. Reaction mixture was diluted with ice water (100 mL), extracted with ethyl acetate (2×50 mL), the organic layer was dried over sodium sulfate and evaporated under reduced pressure to afford crude compound. The crude residue was purified by silica gel column by eluting with 10% methanol and dichloromethane to afford Methyl (2S)-2-((2S)-2-(((1-cyclohexyl-2-phenylethoxy)carbonyl)amino)-4-methylpentanamido)-3-((S)-2-oxopyrrolidin-3-yl)propanoate (7). TLC system: 10% Methanol in dichloromethane Rf: 0.6 LCMS (ESI): m/z=530.7 [M+H]⁺

### 1-Cyclohexyl-2-phenylethyl ((S)-1-(((S)-1-hydroxy-3-((S)-2-oxopyrrolidin-3-yl)propan-2-yl)amino)-4-methyl-1-oxopentan-2-yl)carbamate (8)

To a stirred solution of methyl methyl (2S)-2-((2S)-2-(((1-cyclohexyl-2-phenylethoxy)carbonyl)amino)-4-methylpentanamido)-3-((S)-2-oxopyrrolidin-3-yl)propanoate (7) (0.5 g, 0.944 mmol) in dichloromethane (30 mL) was added 2M LiBH₄ in THF (2 mL, 1.889 mmol) was added at 0° C. and stirred for 2 h. The progress of the reaction was monitored by TLC and LCMS. Reaction mixture was quenched with saturated NH₄Cl solution and extracted with dichloromethane (2×20 mL), dried over sodium sulfate, concentrated under reduced pressure to afford 1-cyclohexyl-2-phenylethyl ((S)-1-(((S)-1-hydroxy-3-((S)-2-oxopyrrolidin-3-yl)propan-2-yl)amino)-4-methyl-1-oxopentan-2-yl)carbamate (8). TLC system: 10% Methanol in dichloromethane Rf: 0.2 LCMS (ESI): m/z 502.48 [M+H]⁺

### 1-Cyclohexyl-2-phenylethyl ((S)-4-methyl-1-oxo-1-(((S)-1-oxo-3-((S)-2-oxopyrrolidin-3-yl)propan-2-yl)amino)pentan-2-yl)carbamate (Compound C237)

To a stirred solution of 1-cyclohexyl-2-phenylethyl ((S)-1-(((S)-1-hydroxy-3-((S)-2-oxopyrrolidin-3-yl)propan-2-yl)amino)-4-methyl-1-oxopentan-2-yl)carbamate (8) (270 mg, 0.5385 mmol) was dissolved in ethyl acetate (10 mL) was added Dess-Martin periodinane (685.1 mg, 1.6157 mmol) at 0° C. and stirred at room temperature for 3 h. Reaction mixture was diluted with dichloromethane (20 mL) and washed with sat. Hypo solution (3×20 mL), sat. NaHCO₃ solution (3×20 mL) and brine (1×20 mL). Organic layer was dried over anhydrous Na₂SO₄, filtered and concentrated to afford crude compound. The crude compound was submitted to Prep HPLC afford 1-cyclohexyl-2-phenylethyl ((S)-4-methyl-1-oxo-1-(((S)-1-oxo-3-((S)-2-oxopyrrolidin-3-yl)propan-2-yl)amino)pentan-2-yl)carbamate (Compound C237). LCMS (ESI): m/z 500.3 [M+H]⁺

### Example 180: Synthesis of Compound C238

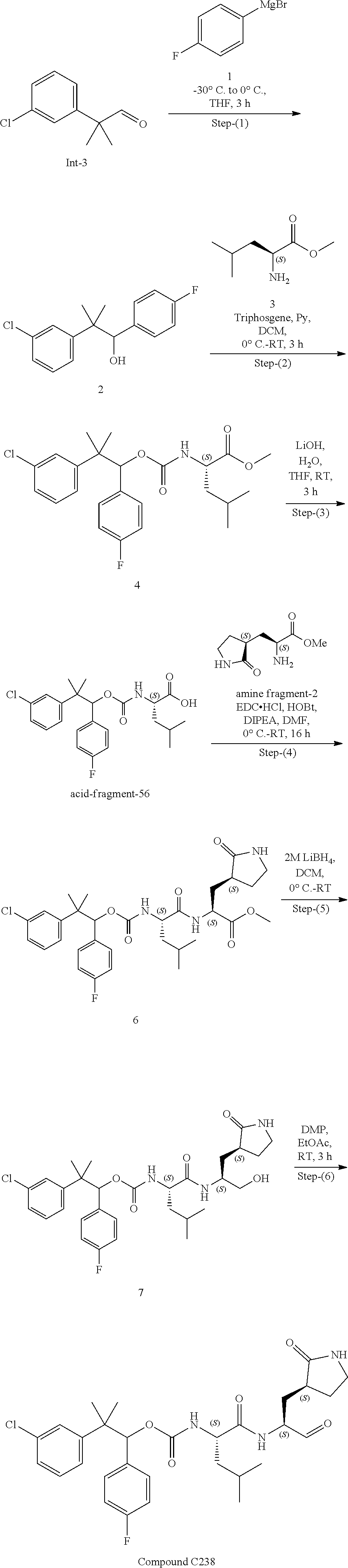

### 2-(3-Chlorophenyl)-1-(4-fluorophenyl)-2-methylpropan-1-ol (2)

To a stirred solution of 2-(3-chlorophenyl)-2-methylpropanal (Int-3) (10 g, 54.945 mmol) in THF (100 mL) was added (4-fluorophenyl)magnesium bromide (54 mL, 109.89 mmol) at −30° C. and the reaction mixture was stirred at 0° C. for 3 h. The progress of the reaction was monitored by TLC. The reaction mixture was quenched with saturated NH₄Cl (100 mL) and filtered through celite pad and washed with ethyl acetate (100 mL). The layers were separated and dried over sodium sulfate then evaporated under reduced pressure. The crude residue was purified by silica gel column by eluting with 10% ethyl acetate in hexane to afford 2-(3-chlorophenyl)-1-(4-fluorophenyl)-2-methylpropan-1-ol (2). TLC system: 10% Ethyl acetate in hexane Rf: 0.3 LCMS (ESI): m/z=261.28 [M−OH]

### Methyl ((2-(3-chlorophenyl)-1-(4-fluorophenyl)-2-methylpropoxy) carbonyl)-L-leucinate (4)

To a stirred solution of 2-(3-chlorophenyl)-1-(4-fluorophenyl)-2-methylpropan-1-ol (2) (5 g, 17.985 mmol) in DCM (30 mL) was added pyridine (6 mL) methyl L-leucinate (3) (4.3 g, 23.381 mmol) followed by triphosgene (2.6 g, 8.992 mmol) at 0° C. slowly and stirred at room temperature for 16 h. The progress of the reaction was monitored by TLC. Reaction mixture was quenched with 1N aq. HCl (100 mL) then extracted with DCM (2×50 mL), washed with sat. NaHCO₃ solution (2×50 mL), organic layer was dried over sodium sulfate and evaporated under reduced pressure. The crude residue was purified by silica gel column by eluting with 15% ethyl acetate in hexane to afford methyl methyl ((2-(3-chlorophenyl)-1-(4-fluorophenyl)-2-methylpropoxy)carbonyl)-L-leucinate (4). TLC system: 10% Ethyl acetate in hexane Rf: 0.3 LCMS (ESI): m/z=472.27 [M+Na]⁺

### ((2-(3-Chlorophenyl)-1-(4-fluorophenyl)-2-methylpropoxy) carbonyl)-L-leucine (5)

To a stirred solution of methyl ((2-(3-chlorophenyl)-1-(4-fluorophenyl)-2-methylpropoxy) carbonyl)-L-leucinate (4) (3.6 g, 8.017 mmol) in THF (20 mL), water (5 mL) was added LiOH.H₂O (1 g, 24.051 mmol) at 0° C. Reaction mixture was stirred at room temperature for 3 h. The progress of the reaction was monitored by TLC and LCMS. The reaction mixture was completely distilled under reduced pressure, crude compound acidified with aq. 1N HCl solution up to pH˜2 and extracted with ethyl acetate (2×40 mL), dried over sodium sulfate, concentrated under reduced pressure to afford ((2-(3-chlorophenyl)-1-(4-fluorophenyl)-2-methylpropoxy)carbonyl)-L-leucine (5). TLC system: 10% Methanol in dichloromethane Rf: 0.2 LCMS (ESI): m/z=458.23 [M+Na]⁺

### Methyl (2S)-2-((2S)-2-(((2-(3-chlorophenyl)-1-(4-fluorophenyl)-2-methylpropoxy) carbonyl) amino)-4-methylpentanamido)-3-((S)-2-oxopyrrolidin-3-yl) propanoate (6)

To a stirred solution of ((2-(3-chlorophenyl)-1-(4-fluorophenyl)-2-methyl propoxy) carbonyl)-L-leucine (5) (1.4 g, 3.21 mmol) in DMF (14 mL) was added EDC.HCl (922 mg, 4.827 mmol), HOBt (651 mg, 4.827 mmol), DIPEA (1.4 mL, 9.654 mmol) and methyl (S)-2-amino-3-((S)-2-oxopyrrolidin-3-yl)propanoate (amine-fragment-2) (718 mg, 3.862 mmol) at 0° C. simultaneously and stirred at room temperature for 16 h. Reaction mixture was diluted with ice water (50 mL), extracted with ethyl acetate (2×50 mL), the organic layer was dried over sodium sulfate and evaporated under reduced pressure to afford crude compound. The crude residue was purified by silica gel column by eluting with 80% Ethyl acetate and pet-ether to afford methyl (2S)-2-((2S)-2-(((2-(3-chlorophenyl)-1-(4-fluorophenyl)-2-methylpropoxy)carbonyl)amino)-4-methylpentanamido)-3-((S)-2-oxopyrrolidin-3-yl)propanoate (6). TLC system: 10% Methanol in dichloromethane Rf: 0.3 LCMS (ESI): m/z=604.65 [M+H]⁺

### 2-(3-Chlorophenyl)-1-(4-fluorophenyl)-2-methylpropyl ((S)-1-(((S)-1-hydroxy-3-((S)-2-oxopyrrolidin-3-yl)propan-2-yl)amino)-4-methyl-1-oxopentan-2-yl)carbamate (7)

To a stirred solution of methyl (2S)-2-((2S)-2-(((2-(3-chlorophenyl)-1-(4-fluorophenyl)-2-methylpropoxy)carbonyl)amino)-4-methylpentanamido)-3-((S)-2-oxopyrrolidin-3-yl)propanoate (6) (300 mg, 0.497 mmol) in DCM (5 mL) was added 2M LiBH₄ in THF (0.5 mL, 0.995 mmol) at 0° C. and the reaction mixture stirred for 2 h at 0° C. The progress of the reaction was monitored by TLC and LCMS. Then reaction mixture was quenched with aq. NH₄Cl (50 mL) and extracted with ethyl acetate (2×20 mL). Organic layer was washed with brine solution (2×20 mL), dried over Na₂SO₄ and concentrated to get compound to afford 2-(3-chlorophenyl)-1-(4-fluorophenyl)-2-methylpropyl((S)-1-(((S)-1-hydroxy-3-((S)-2-oxopyrrolidin-3-yl)propan-2-yl)amino)-4-methyl-1-oxopentan-2-yl)carbamate (7). TLC system: 10% Methanol in DCM Rf: 0.2 LCMS (ESI): m/z 576.64 (M+H)⁺

### 2-(3-Chlorophenyl)-1-(4-fluorophenyl)-2-methylpropyl ((S)-4-methyl-1-oxo-1-(((S)-1-oxo-3-((S)-2-oxopyrrolidin-3-yl) propan-2-yl) amino) pentan-2-yl) carbamate (Compound C238)

To a stirred solution of 2-(3-chlorophenyl)-1-(4-fluorophenyl)-2-methylpropyl ((S)-1-(((S)-1-hydroxy-3-((S)-2-oxopyrrolidin-3-yl)propan-2-yl)amino)-4-methyl-1-oxopentan-2-yl)carbamate (7) (270 mg, 0.469 mmol) was dissolved in ethyl acetate (5 mL) was added Dess-Martin periodinane (398 mg, 0.939 mmol) at 0° C. and stirred at room temperature for 3 h. Reaction mixture was filtered through celite pad and washed with ethyl acetate (20 mL). Filtrate was washed with sat. Hypo solution (3×20 mL), sat. NaHCO₃ solution (3×20 mL). Organic layer was dried over anhydrous Na₂SO₄, filtered and concentrated to afford crude, this crude compound was purified by prep HPLC to afford 2-(3-chlorophenyl)-1-(4-fluorophenyl)-2-methylpropyl ((S)-4-methyl-1-oxo-1-(((S)-1-oxo-3-((S)-2-oxopyrrolidin-3-yl)propan-2-yl)amino)pentan-2-yl)carbamate (Compound C238). TLC system: 10% Methanol in DCM Rf: 0.5 LCMS (ESI): m/z 574.2 (M+H)⁺

### Example 181: Synthesis of Compound C239

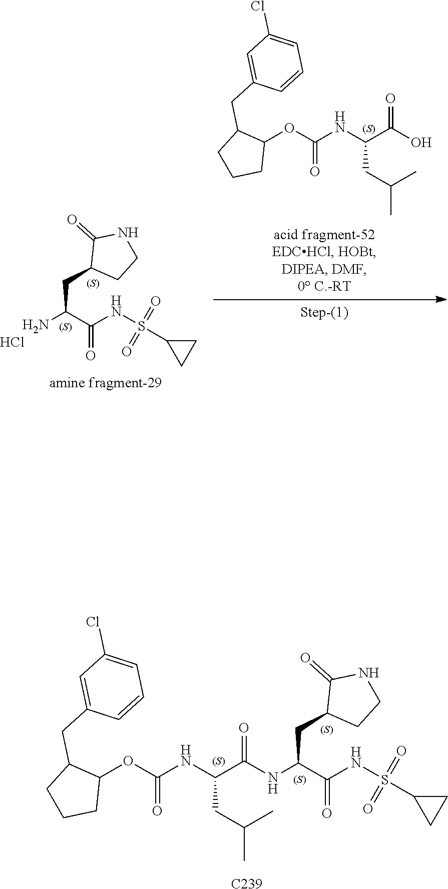

### 2-(3-Chlorobenzyl) cyclopentyl ((S)-1-(((S)-1-(cyclopropanesulfonamido)-1-oxo-3-((S)-2-oxopyrrolidin-3-yl) propan-2-yl) amino)-4-methyl-1-oxopentan-2-yl) carbamate (Compound C239)

To a stirred solution of (((2-(3-chlorobenzyl) cyclopentyl)oxy)carbonyl)-L-leucine (acid fragment-52) (250 mg, 0.681 mmol) DMF (6 mL) was added EDC.HCl (195 mg, 1.021 mmol), HOBt (137 mg, 1.021 mmol), DIPEA (0.3 mL, 2.043 mmol) and (S)-2-amino-N-(cyclopropylsulfonyl)-3-((S)-2-oxopyrrolidin-3-yl) propanamide hydrochloride (amine fragment-29) (254 mg, 0.817 mmol) at 0° C. simultaneously and stirred at room temperature for 16 h. Reaction mixture was diluted with ice water (30 mL) and extracted with ethyl acetate (2×20 mL), dried over sodium sulfate and evaporated under reduced pressure to get crude compound. The crude compound was purified by prep HPLC to afford 2-(3-chlorobenzyl)cyclopentyl((S)-1-(((S)-1-(cyclopropanesulfonamido)-1-oxo-3-((S)-2-oxo pyrrolidin-3-yl) propan-2-yl)amino)-4-methyl-1-oxopentan-2-yl)carbamate (Compound C239). TLC system: 15% Methanol in dichloromethane Rf: 0.1 LCMS (ESI): m/z 625.3 [M+H]⁺

### Example 182: Synthesis of Compound C240

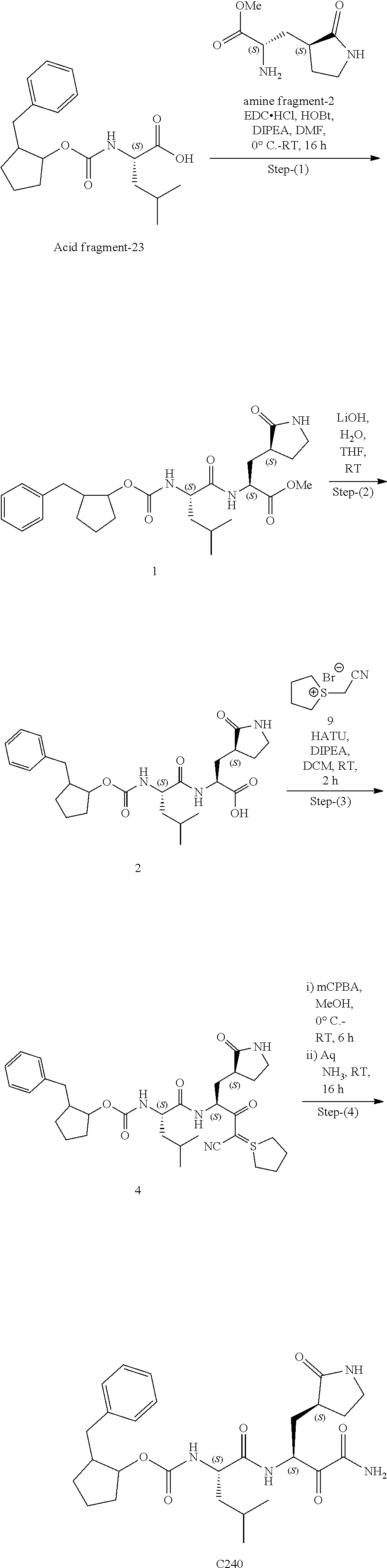

### Methyl (2S)-2-((2S)-2-((((2-benzylcyclopentyl)oxy)carbonyl)amino)-4-methylpentanamido)-3-((S)-2-oxopyrrolidin-3-yl)propanoate (1)

To a stirred solution of (((2-benzylcyclopentyl)oxy)carbonyl)-L-leucine (acid fragment-23) (1 g, 3.003 mmol) in DMF (10 mL) was added HATU (1.7 g, 4.504 mmol), DIPEA (2 mL, 9.009 mmol) and methyl (S)-2-amino-3-((S)-2-oxopyrrolidin-3-yl)propanoate (amine fragment-2) (0.656 g, 3.603 mmol) at 0° C. simultaneously and stirred at room temperature for 16 h. Reaction mixture was diluted with ice water (100 mL), extracted with ethyl acetate (2×50 mL), the organic layer was dried over sodium sulfate and evaporated under reduced pressure to afford crude compound. The crude residue was purified by silica gel column by eluting with 50% ethyl acetate and pet-ether to afford methyl (2S)-2-((2S)-2-((((2-benzylcyclopentyl)oxy)carbonyl)amino)-4-methylpentanamido)-3-((S)-2-oxopyrrolidin-3-yl)propanoate (1). TLC system: 10% Methanol in dichloromethane Rf: 0.3 LCMS (ESI): m/z=502.94 [M+H]⁺

### (2S)-2-((2S)-2-((((2-Benzylcyclopentyl)oxy)carbonyl)amino)-4-methylpentanamido)-3-((S)-2-oxopyrrolidin-3-yl)propanoic acid (2)

To a stirred solution of methyl (2S)-2-((2S)-2-((((2-benzylcyclopentyl)oxy)carbonyl)amino)-4-methylpentanamido)-3-((S)-2-oxopyrrolidin-3-yl)propanoate (1) (700 mg, 1.39 mmol) in THF (10 mL), water (5 mL) was added lithium hydroxide (190 mg, 4.1866 mmol) at room temperature and stirred at room temperature for 3 h. The progress of the reaction was monitored by TLC and LCMS. Reaction mixture completely distilled under reduced pressure, crude compound acidified with aq. 1N HCl solution up to pH˜3 and extracted with ethyl acetate (2×20 mL), dried over sodium sulfate, concentrated under reduced pressure to afford (2S)-2-((2S)-2-((((2-benzylcyclopentyl)oxy)carbonyl)amino)-4-methylpentanamido)-3-((S)-2-oxopyrrolidin-3-yl)propanoic acid (2). TLC system: 15% Methanol in dichloromethane Rf: 0.2 LCMS (ESI): m/z 488.38.31 [M+H]⁺

### 2-Benzylcyclopentyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4)

To a stirred solution of (2S)-2-((2S)-2-((((2-benzylcyclopentyl)oxy)carbonyl)amino)-4-methylpentanamido)-3-((S)-2-oxopyrrolidin-3-yl)propanoic acid (2) (500 mg, 1.025 mmol) DCM (10 mL) added HATU (584 mg, 1.538 mmol), DIPEA (1 mL, 3.076 mmol) and 1-(cyan methyl)tetra hydro-1H-thiophen-1-iumbromide (3) (255 mg, 1.23 mmol) at 0° C. simultaneously and stirred at room temperature for 2 h. Reaction mixture was diluted with ice water (50 mL), extracted with dichloromethane (2×20 mL), dried over sodium sulfate and evaporated under reduced pressure. The crude residue was purified by combi-flash NP, compound eluted at 5% methanol in dichloromethane to afford 2-benzylcyclopentyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4). TLC system: 10% Methanol in dichloromethane Rf: 0.4 LCMS (ESI): m/z 597.73 (M+H)⁺

### 2-Benzylcyclopentyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C240)

To a stirred solution of 2-benzylcyclopentyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (430 mg, 0.72 mmol) in methanol (5 mL) was added m-CPBA (190 mg, 1.08 mmol) at 0° C. and the reaction mixture stirred for 2 h at 0° C. To this was added aq ammonia (5 mL) and stirred at room temperature for 16 h. The progress of the reaction was monitored by TLC and LCMS. Reaction mixture was quenched with sat. NaHCO₃ solution (20 mL) and extracted with DCM (2×20 mL). Organic layer was washed with brine solution (30 mL), dried over Na₂SO₄ and concentrated to get crude compound. The crude compound was purified by prep HPLC to afford 2-benzylcyclopentyl ((S)-1-(((S)-4-amino-3,4-dioxo-1-((S)-2-oxopyrrolidin-3-yl)butan-2-yl)amino)-4-methyl-1-oxopentan-2-yl)carbamate (Compound C240). TLC system: 10% Methanol in dichloromethane Rf: 0.4 LCMS (ESI): m/z 515.2 (M+H)⁺

### Example 183: Synthesis of Compounds C272 and C241

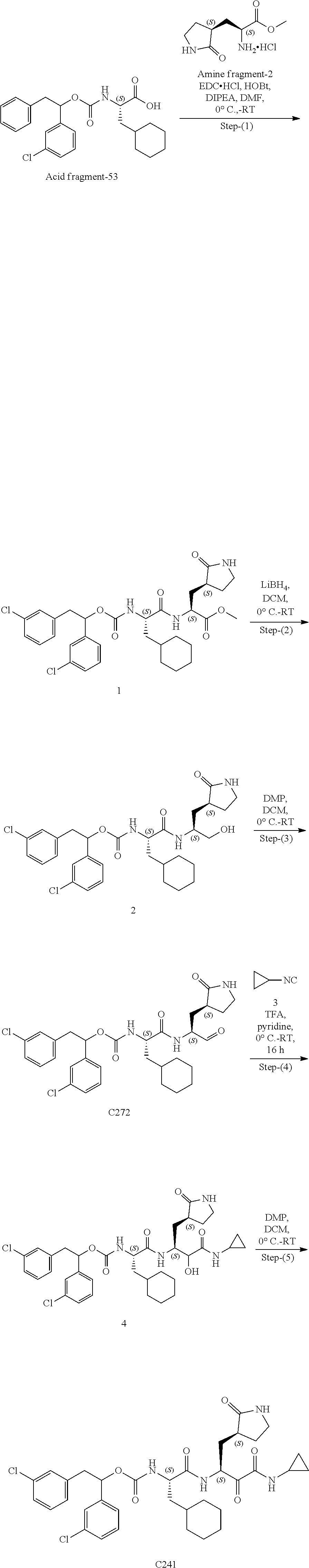

### (2S)-2-(((1,2-Bis(3-chlorophenyl)ethoxy)carbonyl)amino)-3-cyclohexylpropanoic acid (Acid fragment-53)

To a stirred solution of (2S)-2-(((1,2-bis(3-chlorophenyl)ethoxy)carbonyl)amino)-3-cyclohexylpropanoic acid (Acid fragment-53) (2 g, 4.31 mmol) in DMF (10 mL) at 0° C. was added EDC.HCl (1.2 g, 6.28 mmol), HOBT (872 mg, 6.45 mmol), DIPEA (6 mL, 3 Vol.) and methyl (S)-2-amino-3-((S)-2-oxopyrrolidin-3-yl)propanoate hydrochloride (amine fragment 2) (962 mg, 5.17 mmol) simultaneously and stirred at room temperature for 16 h. The progress of the reaction was monitored by TLC and LCMS. After 16 h, reaction mixture was quenched with ice water (20 mL), extracted with ethyl acetate (2×30 mL), the combined organic layer was dried over sodium sulfate and evaporated under reduced pressure. The crude residue was purified by silica gel column by eluting with 50% ethyl acetate in pet ether to afford methyl (2S)-2-((2S)-2-(((1,2-bis(3-chlorophenyl)ethoxy)carbonyl)amino)-3-cyclohexylpropanamido)-3-((S)-2-oxopyrrolidin-3-yl)propanoate. TLC system: 5% Methanol in DCM R_{f}: 0.4 LCMS (ESI): m/z 631.2 (M+H)⁺

### 1,2-Bis(3-chlorophenyl)ethyl ((S)-3-cyclohexyl-1-(((S)-1-hydroxy-3-((S)-2-oxopyrrolidin-3-yl)propan-2-yl)amino)-1-oxopropan-2-yl)carbamate (2)

To a stirred solution of methyl (2S)-2-((2S)-2-(((1,2-bis(3-chlorophenyl)ethoxy)carbonyl)amino)-3-cyclohexylpropanamido)-3-((S)-2-oxopyrrolidin-3-yl)propanoate (1) (1.5 g, 2.37 mmol) in DCM (20 mL) was added 2M LiBH₄ in THF (2.37 mL, 4.75 mmol) at 0° C. and the reaction mixture was stirred for 2 h at room temperature. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated to afford 1,2-bis(3-chlorophenyl)ethyl ((S)-3-cyclohexyl-1-(((S)-1-hydroxy-3-((S)-2-oxopyrrolidin-3-yl)propan-2-yl)amino)-1-oxopropan-2-yl)carbamate (2). TLC system: 5% MeOH in DCM R_{f} 0.2 LCMS (ESI): m/z 604.2 (M+H)⁺

### 1,2-Bis(3-chlorophenyl)ethyl ((S)-3-cyclohexyl-1-oxo-1-(((S)-1-oxo-3-((S)-2-oxopyrrolidin-3-yl)propan-2-yl)amino)propan-2-yl)carbamate (Compound C272)

To a stirred solution of ((S)-3-cyclohexyl-1-(((S)-1-hydroxy-3-((S)-2-oxopyrrolidin-3-yl)propan-2-yl)amino)-1-oxopropan-2-yl)carbamate (2) (500 mg, 0.82 mmol) in ethyl acetate (20 mL) was added Dess-Martin periodinane (1 g, 2.48 mmol) at 0° C. and stirred at room temperature for 3 h. The progress of the reaction was monitored by TLC and LCMS. Reaction mixture was diluted with ethyl acetate (20 mL) and washed with sat. NaHCO₃ solution (3×20 mL) followed by sat. Hypo solution (3×20 mL). Organic layer was dried over anhydrous Na₂SO₄, filtered and concentrated to get crude. It was purified by prep HPLC to afford 1,2-bis(3-chlorophenyl)ethyl ((S)-3-cyclohexyl-1-oxo-1-(((S)-1-oxo-3-((S)-2-oxopyrrolidin-3-yl)propan-2-yl)amino)propan-2-yl)carbamate (Compound C272). TLC system: 5% Methanol in DCM R_{f}: 0.4 LCMS (ESI): m/z 602.2 (M+H)⁺

### 1,2-Bis(3-chlorophenyl)ethyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (4)

To a stirred solution of 1,2-bis(3-chlorophenyl)ethyl ((S)-3-cyclohexyl-1-oxo-1-(((S)-1-oxo-3-((S)-2-oxopyrrolidin-3-yl)propan-2-yl)amino)propan-2-yl)carbamate (Compound C272) (350 mg, 0.69 mmol) was dissolved in DCM (20 mL), added Pyridine (1 mL, 3 vol), isocyanocyclopropane (3) (46 mg, 0.91 mmol) sequentially at 0° C. and stirred for 10 min. To this was added TFA (0.15 mL, 1.31 mmol) at 0° C. and stirred at room temperature for 16 h. The progress of the reaction was monitored by TLC and LCMS. Reaction mixture was quenched with ice water (20 mL) and extracted with dichloromethane (2×15 mL). The organic layer was washed with 1N HCl (3×15 mL), brine solution (3×10 mL). The organic layer was dried over anhydrous Na₂SO₄ and evaporated under reduced pressure to afford crude 1,2-bis(3-chlorophenyl)ethyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (4). TLC system: 5% Methanol/Dichloromethane Rf: 0.5 LCMS (ESI): m/z 687.4 [M+H]⁺

### 1,2-Bis(3-chlorophenyl)ethyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C241)

To a stirred solution of 1,2-bis(3-chlorophenyl)ethyl ((2S)-3-cyclohexyl-1-(((2S)-4-(cyclopropylamino)-3-hydroxy-4-oxo-1-((S)-2-oxopyrrolidin-3-yl)butan-2-yl)amino)-1-oxopropan-2-yl)carbamate (4) (250 mg, 0.36 mmol) in EtOAc (10 mL) was added Dess-Martin periodinane (308 mg, 0.72 mmol) at 0° C. and stirred at room temperature for 3 h. The progress of the reaction was monitored by TLC and LCMS. Reaction mixture was filter through celite pad and washed with Ethyl acetate (25 mL) and filtrate was washed with hypo solution (3×10 mL) followed by saturated NaHCO₃ solution (3×10 mL). Organic layer was dried over anhydrous Na₂SO₄, filtered and concentrated to get crude residue. The crude compound was purified by prep HPLC to afford 1,2-bis(3-chlorophenyl)ethyl ((S)-3-cyclohexyl-1-(((S)-4-(cyclopropylamino)-3,4-dioxo-1-((S)-2-oxopyrrolidin-3-yl)butan-2-yl)amino)-1-oxopropan-2-yl)carbamate (Compound C241). TLC system: 10% Methanol/Dichloromethane Rf: 0.4 LCMS (ESI): m/z 685.2 (M+H)⁺

### Example 184: Synthesis of Compounds C282 and C242

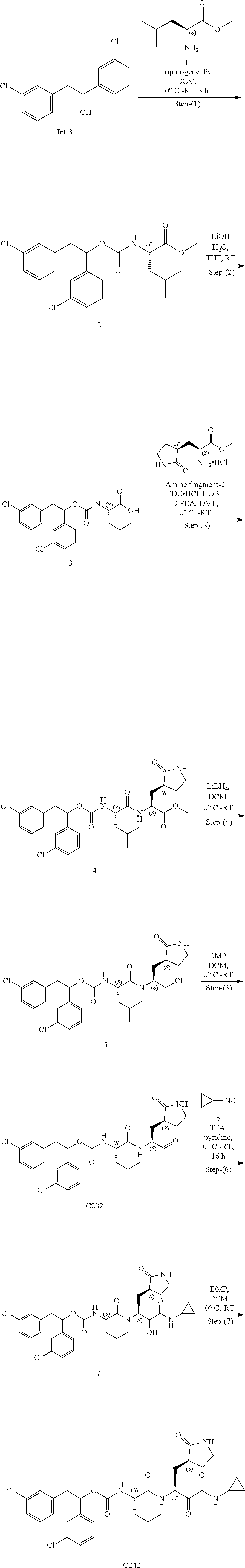

### Methyl ((1,2-bis(3-chlorophenyl)ethoxy)carbonyl)-L-leucinate (2)

To a stirred solution of 1,2-bis(3-chlorophenyl)ethan-1-ol (Int-3) (2.7 g, 10.11 mmol), methyl (S)-methyl L-leucinate HCl (2.2 g, 12.13 mmol) in DCM (40 mL) was added pyridine (8.5 mL, 3 vol) followed by triphosgene (1.4 g, 4.72 mmol) at 0° C. and stirred at room temperature for 3 h. The progress of the reaction was monitored by TLC and LCMS. Reaction mixture was quenched with 2N HCl (50 mL), extracted with DCM (2×40 mL). The combined organic layer was dried over sodium sulfate, filtered and evaporated under reduced pressure. The crude residue was purified by combi-flash, compound eluted at 10% ethyl acetate in pet ether to afford methyl ((1,2-bis(3-chlorophenyl)ethoxy)carbonyl)-L-leucinate (2). TLC system: 30% Ethyl acetate in hexane Rf: 0.3 LCMS (ESI): m/z 477.1 [M+Na]⁺

### ((1,2-Bis(3-chlorophenyl)ethoxy)carbonyl)-L-leucine (3)

To a stirred solution of methyl ((1,2-bis(3-chlorophenyl)ethoxy)carbonyl)-L-leucinate (2) (2.5 g, 5.24 mmol) in THF (20 mL), water (20 mL) was added lithium hydroxide (0.64 g, 15.72 mmol) at 0° C. and stirred at room temperature for 2 h. The progress of the reaction was monitored by TLC and LCMS. Reaction mixture completely distilled under reduced pressure, crude compound acidified with aq. 1N HCl solution up to pH˜3 and extracted with ethyl acetate (2×40 mL), dried over sodium sulfate, concentrated under reduced pressure to afford ((1,2-bis(3-chlorophenyl)ethoxy)carbonyl)-L-leucine (3). TLC system: 100% EtOAc Rf: 0.1 LCMS (ESI): m/z 423.1 [M+Na]⁺

### Methyl (2S)-2-((2S)-2-(((1,2-bis (3-chlorophenyl) ethoxy) carbonyl) amino)-4-methylpentanamido)-3-((S)-2-oxopyrrolidin-3-yl) propanoate (4)

To a stirred solution of ((1,2-bis(3-chlorophenyl)ethoxy)carbonyl)-L-leucine (3) (2 g, 4.728 mmol) in DMF at 0° C. was added EDC.HCl (1.35 g, 7.092 mmol), HOBT (957 mg, 7.092 mmol), DIPEA (4 mL, 3 Vol.) and methyl (S)-2-amino-3-((S)-2-oxopyrrolidin-3-yl)propanoate hydrochloride (amine fragment 2) (1.055 g, 5.673 mmol) simultaneously and stirred at room temperature for 16 h. The progress of the reaction was monitored by TLC and LCMS. After 16 h, reaction mixture was quenched with ice water (20 mL), extracted with ethyl acetate (2×30 mL), the combined organic layer was dried over sodium sulfate and evaporated under reduced pressure. The crude residue was purified by silica gel column by eluted with 50% ethyl acetate in pet ether to afford Methyl (2S)-2-((2S)-2-(((1,2-bis (3-chlorophenyl) ethoxy) carbonyl) amino)-4-methylpentanamido)-3-((S)-2-oxopyrrolidin-3-yl) propanoate (4). TLC system: 5% Methanol in DCM R_{f}: 0.4 LCMS (ESI): m/z 591.9 (M+Na)⁺

### 1,2-Bis(3-chlorophenyl)ethyl ((S)-1-(((S)-1-hydroxy-3-((S)-2-oxopyrrolidin-3-yl)propan-2-yl)amino)-4-methyl-1-oxopentan-2-yl)carbamate (5)

To a stirred solution of methyl (2S)-2-((2S)-2-(((1,2-bis (3-chlorophenyl) ethoxy) carbonyl) amino)-4-methylpentanamido)-3-((S)-2-oxopyrrolidin-3-yl) propanoate (4) (1.5 g, 2.538 mmol) in DCM (20 mL) was added 2M LiBH₄ in THF (2.53 mL, 5.076 mmol) at 0° C. and the reaction mixture was stirred for 2 h at room temperature. The progress of the reaction was monitored by TLC and LCMS. After 2 h, reaction mixture was quenched with water (20 mL) and extracted with DCM (2×30 mL). Organic layer was washed with brine solution (30 mL), and combined organic layer was dried over Na₂SO₄ and concentrated The crude residue was purified by silica gel column by eluted with 90% ethyl acetate in pet ether to afford 1,2-bis(3-chlorophenyl)ethyl 1,2-bis(3-chlorophenyl)ethyl ((S)-1-(((S)-1-hydroxy-3-((S)-2-oxopyrrolidin-3-yl)propan-2-yl)amino)-4-methyl-1-oxopentan-2-yl)carbamate (5). TLC system: 5% Methanol in DCM R_{f} 0.2 LCMS (ESI): m/z 564.2 (M+H)⁺

### 1,2-Bis(3-chlorophenyl)ethyl ((S)-4-methyl-1-oxo-1-(((S)-1-oxo-3-((S)-2-oxopyrrolidin-3-yl)propan-2-yl)amino)pentan-2-yl)carbamate (Compound C282)

To a stirred solution of 1,2-bis(3-chlorophenyl)ethyl 1,2-bis(3-chlorophenyl)ethyl ((S)-1-(((S)-1-hydroxy-3-((S)-2-oxopyrrolidin-3-yl)propan-2-yl)amino)-4-methyl-1-oxopentan-2-yl) carbamate (5) (500 mg, 0.888 mmol) in ethyl acetate (20 mL) was added Dess-Martin periodinane (1.1 g, 2.66 mmol) at 0° C. and stirred at room temperature for 3 h. The progress of the reaction was monitored by TLC and LCMS. Reaction mixture was diluted with ethyl acetate (20 mL) and washed with sat. NaHCO₃ solution (3×20 mL) followed by sat. Hypo solution (3×20 mL). Organic layer was dried over anhydrous Na₂SO₄, filtered and concentrated crude purified by prep-HPLC to afford 1,2-bis(3-chlorophenyl)ethyl((S)-4-methyl-1-oxo-1-(((S)-1-oxo-3-((S)-2-oxopyrrolidin-3-yl) propan-2-yl)amino)pentan-2-yl)carbamate (Compound C282). TLC system: 5% Methanol in DCM R_{f}: 0.4 LCMS (ESI): m/z 562.1 (M+H)⁺

### 1,2-Bis(3-chlorophenyl)ethyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (7)

To a stirred solution of 1,2-bis(3-chlorophenyl)ethyl ((S)-4-methyl-1-oxo-1-(((S)-1-oxo-3-((S)-2-oxopyrrolidin-3-yl)propan-2-yl)amino)pentan-2-yl)carbamate (Compound C282) (350 mg, 0.623 mmol) was dissolved in DCM (10 mL), added Pyridine (1 mL, 3 vol), isocyanocyclopropane (6) (61 mg, 0.91 mmol) sequentially at 0° C. and stirred for 10 min. To this was added TFA (0.14 mL, 2.49 mmol) at 0° C. and stirred at room temperature for 16 h. The progress of the reaction was monitored by TLC and LCMS. Reaction mixture was quenched with ice water (20 mL) and extracted with dichloromethane (2×15 mL). The organic layer was washed with 1N HCl (3×15 mL), brine solution (3×10 mL). The organic layer was dried over anhydrous Na₂SO₄ and evaporated under reduced pressure to afford crude 1,2-bis(3-chlorophenyl)ethyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl)carbamate (7). TLC system: 5% Methanol/Dichloromethane Rf: 0.5 LCMS (ESI): m/z 647.5 [M+H]⁺

### 1,2-Bis(3-chlorophenyl)ethyl ((S)-1-(((S)-4-(cyclopropylamino)-3,4-dioxo-1-((S)-2-oxopyrrolidin-3-yl)butan-2-yl)amino)-4-methyl-1-oxopentan-2-yl)carbamate (Compound C242)

To a stirred solution of 1,2-bis(3-chlorophenyl)ethyl ((2S)-1-(((2S)-4-(cyclopropylamino)-3-hydroxy-4-oxo-1-((S)-2-oxopyrrolidin-3-yl)butan-2-yl)amino)-4-methyl-1-oxopentan-2-yl) carbamate (7) (200 mg, 0.309 mmol) in EtOAc (10 mL) was added Dess-Martin periodinane (393 mg, 0.928 mmol) at 0° C. and stirred at room temperature for 3 h. The progress of the reaction was monitored by TLC and LCMS. Reaction mixture was filter through celite pad and washed with Ethyl acetate (25 mL) and filtrate was washed with hypo solution (3×10 mL) followed by saturated NaHCO₃ solution (3×10 mL). Organic layer was dried over anhydrous Na₂SO₄, filtered and concentrated to get crude residue. The crude compound was purified by prep HPLC to afford 1,2-bis(3-chlorophenyl)ethyl((S)-1-(((S)-4-(cyclopropylamino)-3,4-dioxo-1-((S)-2-oxopyrrolidin-3yl)butan-2-yl)amino)-4-methyl-1-oxopentan-2-yl)carbamate (Compound C242). TLC system: 10% Methanol/Dichloromethane Rf: 0.4 LCMS (ESI): m/z 645.2 (M+H)⁺

### Example 185: Synthesis of Compound C243

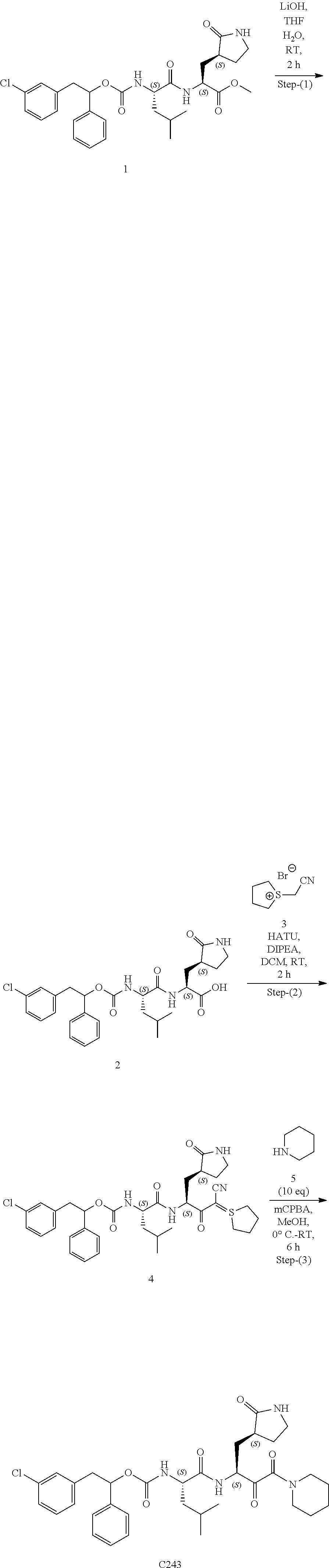

### (2S)-2-((2S)-2-(((2-(3-chlorophenyl)-1-phenylethoxy) carbonyl) amino)-4-methylpentanamido)-3-((S)-2-oxopyrrolidin-3-yl) propanoic acid (2)

To a stirred solution of methyl (2S)-2-((2S)-2-(((2-(3-chlorophenyl)-1-phenylethoxy)carbonyl)amino)-4-methylpentanamido)-3-((S)-2-oxopyrrolidin-3-yl)propanoate (1) (1.2 g, 2.154 mmol) in THF (10 mL), water (5 mL) was added lithium hydroxide (271 mg, 6.460 mmol) at room temperature and stirred at room temperature for 3 h. The progress of the reaction was monitored by TLC and LCMS. Reaction mixture completely distilled under reduced pressure, crude compound acidified with aq. 1N HCl solution up to pH˜2 and extracted with ethyl acetate (2×30 mL), dried over sodium sulfate, concentrated under reduced pressure to afford (2S)-2-((2S)-2-(((2-(3-chlorophenyl)-1-phenyl ethoxy)carbonyl)amino)-4-methylpentanamido)-3-((S)-2-oxopyrrolidin-3-yl)propanoic acid (2). TLC system: 100% Ethyl acetate Rf: 0.1 LCMS (ESI): m/z 544.46 [M+H]⁺

### 2-(3-Chlorophenyl)-1-phenylethyl ((S)-1-(((S)-4-cyano-3-oxo-1-((S)-2-oxopyrrolidin-3-yl)-4-(tetrahydro-1l4-thiophen-1-ylidene) butan-2-yl) amino)-4-methyl-1-oxopentan-2-yl) carbamate (4)

To a stirred solution of (2S)-2-((2S)-2-(((2-(3-chlorophenyl)-1-phenyl ethoxy)carbonyl)amino)-4-methylpentanamido)-3-((S)-2-oxopyrrolidin-3-yl)propanoic acid (2) (800 mg, 1.472 mmol) in DCM (10 mL) was added HATU (618 mg, 2.209 mmol), DIPEA (0.81 mL, 4.418 mmol) and 1-(cyan methyl)tetra hydro-1H-thiophen-1-iumbromide (3) (455 mg, 2.209 mmol) at 0° C. simultaneously and stirred at room temperature for 2 h. Reaction mixture was diluted with ice water (30 mL), extracted with dichloromethane (2×25 mL), dried over sodium sulfate and evaporated under reduced pressure. The crude residue was purified by combi-flash NP, compound eluted at 5% methanol in dichloromethane to afford 2-(3-chlorophenyl)-1-phenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4). TLC system: 10% Methanol in dichloromethane Rf: 0.5 LCMS (ESI): m/z 653.67 [M+H]⁺

### 2-(3-Chlorophenyl)-1-phenylethyl ((S)-1-(((S)-3,4-dioxo-1-((S)-2-oxopyrrolidin-3-yl)-4-(piperidin-1-yl) butan-2-yl) amino)-4-methyl-1-oxopentan-2-yl) carbamate (Compound C243)

To a stirred solution of 2-(3-chlorophenyl)-1-phenylethyl ((S)-1-(((S)-4-cyano-3-oxo-1-((S)-2-oxopyrrolidin-3-yl)-4-(tetrahydro-1l4-thiophen-1-ylidene)butan-2-yl)amino)-4-methyl-1-oxopentan-2-yl)carbamate (4) (350 mg, 0.535 mmol) in methanol (5 mL) was added m-CPBA (185 mg, 1.071 mmol) at 0° C. and the reaction mixture stirred for 2 h at 0° C. To this was added piperidine (4 mL) and stirred at room temperature for 16 h. The progress of the reaction was monitored by TLC and LCMS. Reaction mixture was diluted with dichloromethane and washed with sat. NaHCO₃ solution (3×20 mL). Organic layer was washed with brine solution (30 mL), dried over Na₂SO₄ and concentrated to get crude compound. The crude compound was purified by prep HPLC to afford 2-(3-chlorophenyl)-1-phenylethyl ((S)-1-(((S)-3,4-dioxo-1-((S)-2-oxopyrrolidin-3-yl)-4-(piperidin-1-yl)butan-2-yl)amino)-4-methyl-1-oxopentan-2-yl)carbamate (Compound C243). TLC system: 10% Methanol in dichloromethane Rf: 0.3 LCMS (ESI): m/z 639.2 [M+H]⁺

### Example 186: Synthesis of Compound C244

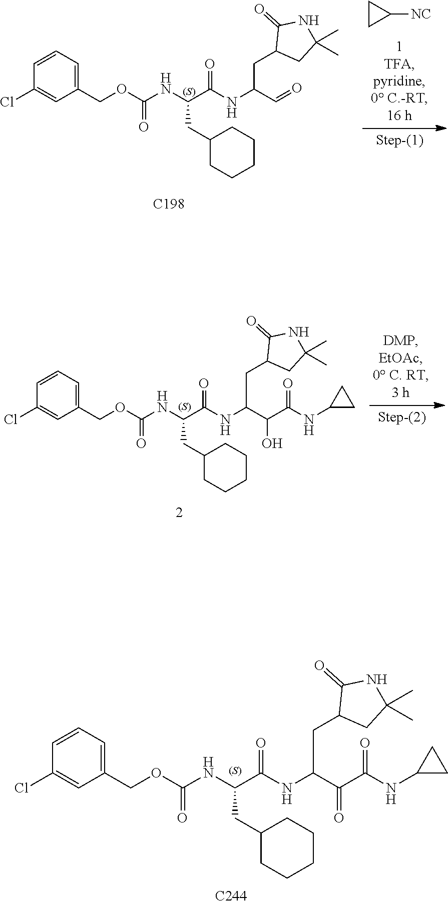

### 3-Chlorobenzyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl)carbamate (2)

To a stirred solution of 3-chlorobenzyl ((2S)-3-cyclohexyl-1-((1-(5,5-dimethyl-2-oxo pyrrolidin-3-yl)-3-oxopropan-2-yl)amino)-1-oxopropan-2-yl)carbamate (Compound C198) (230 mg, 0.455 mmol) was dissolved in DCM (10 mL) added Pyridine (1 mL, 4 vol), isocyanocyclopropane (2) (45 mg, 0.682 mmol) followed by TFA (0.13 mL, 1.09 mmol) at 0° C. and stirred at room temperature for 16 h. The progress of the reaction was monitored by TLC and LCMS. Reaction mixture was diluted with dichloromethane and washed with 1N HCl (2×15 mL) followed by brine (20 mL). Organic layer was dried over anhydrous Na₂SO₄ and evaporated under reduced pressure to afford crude 3-chlorobenzyl ((2S)-3-cyclohexyl-1-((4-(cyclopropylamino)-1-(5,5-dimethyl-2-oxopyrrolidin-3-yl)-3-hydroxy-4-oxo butan-2-yl) amino)-1-oxopropan-2-yl) carbamate (2). TLC system: 10% Methanol in dichloromethane Rf: 0.5 LCMS (ESI): m/z 591.35 [M+H]⁺

### 3-Chlorobenzyl ((2S)-3-cyclohexyl-1-((4-(cyclopropylamino)-1-(5,5-dimethyl-2-oxopyrrolidin-3-yl)-3,4-dioxobutan-2-yl) amino)-1-oxopropan-2-yl) carbamate (Compound C244)

To a stirred solution of 3-chlorobenzyl ((2S)-3-cyclohexyl-1-((4-(cyclopropylamino)-1-(5,5-dimethyl-2-oxopyrrolidin-3-yl)-3-hydroxy-4-oxobutan-2-yl)amino)-1-oxopropan-2-yl) carbamate (2) (100 mg, 0.169 mmol) in ethyl acetate (10 mL) was added Dess-Martin periodinane (143 mg, 0.338 mmol) at 0° C. and stirred at room temperature for 3 h. The progress of the reaction was monitored by TLC and LCMS. Reaction mixture was filter through celite pad and washed with ethyl acetate (25 mL) and filtrate was washed with hypo solution (3×20 mL) followed by saturated NaHCO₃ solution (3×20 mL). Organic layer was dried over anhydrous Na₂SO₄, filtered and concentrated to get crude residue. The crude compound was purified by prep HPLC to afford 3-chlorobenzyl ((2S)-3-cyclohexyl-1-((4-(cyclopropylamino)-1-(5,5-dimethyl-2-oxopyrrolidin-3-yl)-3,4-dioxo butan-2-yl) amino)-1-oxopropan-2-yl) carbamate (Compound C244). TLC system: 10% Methanol in dichloromethane Rf: 0.4 LCMS (ESI): m/z 589.3 [M+H]⁺

### Testing of Activity of Compounds

### In Vitro Antiviral Assays

Norovirus Antiviral Assays:

Cell-based antiviral assays: The antiviral effects of inhibitors are examined in the Norwalk virus replicon harboring cells (HG23 cells). Briefly, confluent and semi-confluent cells are incubated with medium containing DMSO (<0.1%) or each compound (up to 100 μM) for 48 h. After the incubation, total RNA was extracted and viral genome is quantitated with real-time quantitative RT-PCR (qRT-PCR). The EC₅₀ values are determined by GraphPadPrism software. In addition to Norwalk virus replicon, the CPE (cytopathic effect) antiviral activities of the inhibitors are determined using FCoV (feline coronavirus), MERS-CoV (Middle East respiratory syndrome-related coronavirus), SARS-CoV (severe acute respiratory syndrome-related coronavirus), human coronavirus 229E, murine norovirus, and human rhinovirus.

Viral protease assays: The antiviral activities of inhibitors were determined by FRET (Fluorescence Resonance Energy Transfer) assay. Purified viral protease was incubated with the protease substrate peptide (Edans-DFHLQ/GP-Dabcyl) and inhibitor, and IC₅₀ values were subsequently determined by the fluorescence signals.

The Noro-Norwalk FRET protease assay was performed in 50 mM HEPES-Na pH 8, 50 mM NaCl, 0.4 mM EDTA, 4% glycerol, and 6 mM DTT. A self-quenching peptide substrate [5-FAM]-EPDFHLQGPEDLAKK-[TAMRA] was custom synthesized by Anaspec. Compounds were diluted in 3-fold serial dilutions to final concentrations of 200 μM to 10.2 nM. Noro protease was added to a final concentration of 1-7 μM, depending on the enzyme activity level, and the peptide substrate was added to a final concentration of 10 μM. The assay was incubated 90 minutes at 37° C. and read in a Perkin Elmer Envision with excitation at 473 nm and emission measurement at 519 nm.

The SARS2 FRET protease assay was performed in 20 mM HEPES-Na pH 7, 120 mM NaCl, 0.4 mM EDTA, 0.01% Triton, 5% glycerol, and 4 mM DTT. A self-quenching peptide substrate 5-FAM-TSA VLQ SGF RKK (5TAMRA)-NH2 was custom synthesized by Anaspec. Compounds were diluted in 3-fold serial dilutions to final concentrations of 20 μM to 1.0 nM. SARS2 protease was added to a final concentration of 25 nM, depending on the enzyme activity level, and the peptide substrate was added to a final concentration of 1.3 μM. The assay was incubated 30 minutes at 30° C. and read in a Perkin Elmer Envision with excitation at 473 nm and emission measurement at 519 nm.

Results of a Norovirus protease inhibition assay are presented in Table D below.

TABLE D

| Compound | | |
|---|---|---|
| no. | IC₅₀ (μM) | |
| C32 | 4.2 | μM |
| C37 | 26.0 | μM |
| C38 | 3.7 | μM |
| C43 | 5.9 | μM |
| C52 | 9.2 | μM |
| C54 | 7.0 | μM |
| C66 | 10.9 | μM |
| C67 | 9.1 | μM |
| C68 | >200 | |
| C69 | >200 | |
| C70 | 5.2 | μM |
| C71 | 87.0 | μM |
| C72 | 2.3 | μM |
| C73 | 39.1 | μM |
| C74 | 16.4 | μM |
| C75 | 8.1 | μM |
| C76 | 10.5 | μM |
| C77 | 32.6 | μM |
| C78 | >200 | |
| C79 | >200 | |
| C80 | >200 | |
| C81 | 23.9 | μM |
| C82 | 126.6 | μM |
| C83 | >200 | |
| C84 | 190.5 | μM |
| C85 | 143.4 | μM |
| C86 | >200 | |
| C87 | 208.7 | μM |
| C88 | >200 | |
| C89 | 99.2 | μM |
| C90 | 136.2 | μM |
| C91 | >200 | |
| C92 | 36.3 | μM |
| C93 | 209.3 | μM |
| C94 | 156.3 | μM |
| C95 | 115.9 | μM |
| C96 | 4.1 | μM |
| C97 | 66.9 | μM |
| C98 | >200 | |
| C99 | >200 | |
| C100 | >200 | |
| C101 | >200 | |
| C102 | 6.9 | μM |
| C103 | 21.3 | μM |
| C104 | 2.8 | μM |
| C105 | 2.7 | μM |
| C106 | 1.8 | μM |
| C107 | 85.5 | μM |
| C108 | 0.979 | μM |
| C109 | 1.4 | μM |
| C110 | 1.3 | μM |
| C111 | 40.7 | μM |
| C112 | 109.2 | μM |
| C113 | 104.5 | μM |
| C114 | 1.7 | μM |
| C115 | 2.2 | μM |
| C116 | 98.1 | μM |
| C117 | 77.7 | μM |
| C118 | 118.8 | μM |
| C119 | 67.6 | μM |
| C120 | 62.6 | μM |
| C121 | 106.8 | μM |
| C122 | 35.2 | μM |
| C123 | 121.5 | μM |
| C124 | 36.5 | μM |
| C125 | 47.2 | μM |
| C126 | >200 | |
| C127 | 5.7 | μM |
| C128 | 155.6 | μM |
| C129 | >200 | |
| C130 | >200 | |
| C131 | 193.1 | μM |
| C132 | 6.0 | μM |
| C133 | 0.743 | μM |
| C134 | 171.3 | μM |
| C135 | 191.5 | μM |
| C136 | 3.2 | μM |
| C137 | 9.9 | μM |
| C138 | 92.9 | μM |
| C139 | 162.8 | μM |
| C140 | 76.0 | μM |
| C141 | 9.6 | μM |
| C142 | >200 | |
| C143 | 3.7 | μM |
| C144 | 140.2 | μM |
| C145 | >200 | |
| C146 | >200 | |
| C147 | >200 | |
| C148 | 58.7 | μM |
| C149 | >200 | |
| C150 | >200 | |
| C151 | 130.7 | μM |
| C152 | 2.9 | μM |
| C153 | >200 | |
| C154 | 76.1 | μM |
| C155 | 86.4 | μM |
| C156 | 6.1 | μM |
| C157 | 3.1 | μM |
| C158 | 100.4 | μM |
| C159 | 75.3 | μM |
| C160 | 4.3 | μM |
| C161 | >200 | |
| C162 | 8.3 | μM |
| C163 | 62.8 | μM |
| C164 | 142.6 | μM |
| C165 | 5.5 | μM |
| C166 | 13.9 | μM |
| C167 | 38.6 | μM |
| C168 | >200 | |
| C169 | >200 | |
| C170 | 52.5 | μM |
| C171 | >200 | |
| C172 | >200 | |
| C173 | 44.0 | μM |
| C174 | 57.9 | μM |
| C175 | 38.3 | μM |
| C176 | 69.7 | μM |
| C177 | 100.0 | μM |
| C178 | 3.5 | μM |
| C179 | 3.1 | μM |
| C180 | 6.1 | μM |
| C181 | 3.3 | μM |
| C182 | 106.1 | μM |
| C183 | 16.2 | μM |
| C184 | 81.7 | μM |
| C185 | 28.2 | μM |
| C186 | 30.8 | μM |
| C187 | 81.9 | μM |
| C188 | 46.6 | μM |
| C189 | 3.3 | μM |
| C190 | 37.1 | μM |
| C191 | 91.9 | μM |
| C192 | 27.7 | μM |
| C193 | 3.8 | μM |
| C194 | >200 | |
| C195 | >200 | |
| C196 | 26.3 | μM |
| C197 | 155.0 | μM |
| C198 | 3.3 | μM |
| C199 | 92.8 | μM |
| C200 | 1.2 | μM |
| C201 | 2.7 | μM |
| C202 | 7.3 | μM |
| C203 | 1.5 | μM |
| C204 | 1.8 | μM |
| C205 | 183.8 | μM |
| C206 | 76.3 | μM |
| C207 | 106.0 | μM |
| C208 | >200 | |
| C209 | 88.0 | μM |
| C210 | >200 | |
| C211 | 57.8 | μM |
| C212 | 5.3 | μM |
| C213 | 18.9 | μM |
| C214 | 4.6 | μM |
| C215 | >200 | |
| C216 | >200 | |
| C217 | >200 | |
| C218 | >200 | |
| C219 | >200 | |
| C220 | >200 | |
| C221 | >200 | |
| C222 | 13.8 | μM |
| C223 | 22.6 | μM |
| C224 | 7.8 | μM |
| C225 | 7.0 | μM |
| C226 | 169.2 | μM |
| C227 | >200 | |
| C228 | 25.8 | μM |
| C229 | 4.6 | μM |
| C230 | 13.4 | μM |
| C231 | 204.1 | μM |
| C232 | >200 | |
| C233 | >200 | |
| C234 | 124.4 | μM |
| C235 | 8.9 | μM |
| C236 | 185.1 | μM |
| C237 | 8.6 | μM |
| C238 | 1.6 | μM |
| C239 | Act | |
| C240 | >200 | |
| C241 | 85.7 | μM |
| C242 | 33.9 | μM |
| C243 | >200 | |
| C244 | 97.6 | μM |
| C245 | >200 | |
| C246 | 52.1 | μM |
| C247 | 182.7 | μM |
| C248 | >200 | |
| C249 | >200 | |
| C250 | 11.7 | μM |
| C251 | 58.2 | μM |
| C252 | 0.973 | μM |
| C253 | 1.8 | μM |
| C254 | >200 | |
| C255 | >200 | |
| C256 | >200 | |
| C257 | >200 | |
| C258 | 42.9 | μM |
| C259 | 4.4 | μM |
| C260 | >200 | |
| C261 | 192.6 | μM |
| C262 | 3.2 | μM |
| C263 | 1.0 | μM |
| C264 | 12.7 | μM |
| C265 | 10.4 | μM |
| C266 | 0.796 | μM |
| C267 | 7.5 | μM |
| C268 | 0.549 | μM |
| C269 | 2.2 | μM |
| C270 | 105.8 | μM |
| C271 | >200 | |
| C272 | 16.4 | μM |
| C273 | >200 | |
| C274 | >200 | |
| C275 | 1.5 | μM |
| C276 | 3.5 | μM |
| C277 | 33.4 | μM |
| C278 | 3.5 | μM |
| C279 | >200 | |
| C280 | >200 | |
| C281 | >200 | |
| C282 | 1.0 | μM |
| C283 | >200 | |
| C284 | 201.6 | μM |
| C285 | 27.1 | μM |
| C286 | 15.2 | μM |
| C287 | >200 | |
| C288 | 119.1 | μM |
| C289 | 86.0 | μM |
| C290 | 93.2 | μM |
| C291 | 2.5 | μM |
| C292 | 90.5 | μM |
| C293 | 8.7 | μM |
| C294 | 177.3 | μM |
| C295 | 5.1 | μM |
| C296 | 115.5 | μM |
| C297 | 217.1 | μM |
| C298 | 217.9 | μM |

Results of a SARS-CoV-2 protease inhibition assay are presented in Table E below.

TABLE E

| Compound | | |
|---|---|---|
| no. | IC₅₀ (μM) | |
| C1 | >200 | |
| C2 | 71.0 | μM |
| C3 | >200 | |
| C4 | >200 | |
| C5 | >200 | |
| C6 | >200 | |
| C7 | >200 | |
| C8 | >200 | |
| C9 | >200 | |
| C10 | >200 | |
| C11 | >200 | |
| C12 | >200 | |
| C13 | >200 | |
| C14 | >200 | |
| C15 | >200 | |
| C16 | 37.8 | μM |
| C17 | 2.7 | μM |
| C18 | 49.25 | μM |
| C19 | 94.3 | μM |
| C20 | >200 | |
| C21 | >200 | |
| C22 | 3.8 | μM |
| C23 | >200 | |
| C24 | >200 | |
| C25 | 11.1 | μM |
| C26 | >200 | |
| C27 | >200 | |
| C28 | >200 | |
| C29 | >200 | |
| C30 | 42.3 | μM |
| C31 | >200 | |
| C32 | 0.023 | μM |
| C33 | >200 | |
| C34 | 0.1 | μM |
| C35 | 18.9 | μM |
| C36 | 2.5 | μM |
| C37 | 0.1373 | μM |
| | 0.2621 | μM |
| C38 | 0.016 | μM |
| C39 | 0.153 | μM |
| C40 | 0.1735 | μM |
| | 0.2214 | μM |
| C41 | 0.308 | μM |
| C42 | 0.356 | μM |
| C43 | 0.056 | μM |
| C44 | 0.331 | μM |
| C45 | 0.304 | μM |
| C46 | 1.382 | μM |
| C47 | 0.073 | μM |
| C48 | 0.241 | μM |
| C49 | 0.266 | μM |
| C50 | 0.119 | μM |
| C51 | 0.115 | μM |
| C52 | 0.5 | μM |
| C53 | 0.04 | μM |
| C54 | 0.1 | μM |
| C55 | 0.3 | μM |
| C56 | 0.6 | μM |
| C57 | 0.8 | μM |
| C58 | 0.1 | μM |
| C59 | 0.1 | μM |
| C60 | 0.6 | μM |
| C61 | 0.0 | μM |
| C62 | 4.6 | μM |
| C63 | 1.6 | μM |
| C64 | 0.1 | μM |
| C65 | 26.9 | μM |
| C66 | 0.1 | μM |
| C67 | 0.3 | μM |
| C68 | 2.1 | μM |
| C69 | 26.8 | μM |
| C70 | 0.1 | μM |
| C71 | 1.6 | μM |
| C72 | 0.01 | μM |
| C73 | 0.60 | μM |
| C74 | 0.14 | μM |
| C75 | 0.0012 | μM |
| C76 | 0.38 | μM |
| C77 | 0.6 | μM |
| C78 | 0.5 | μM |
| C79 | 3.8 | μM |
| C80 | 0.9 | μM |
| C81 | 0.2 | μM |
| C82 | 4.4 | μM |
| C83 | 1.3 | μM |
| C84 | 9.2 | μM |
| C85 | 17.8 | μM |
| C86 | 0.9 | μM |
| C87 | 0.7 | μM |
| C88 | 1.3 | μM |
| C89 | 0.3 | μM |
| C90 | 0.1 | μM |
| C91 | >20 | |
| C92 | 0.2 | μM |
| C93 | >20 | |
| C94 | 2.6 | μM |
| C95 | 7.0 | μM |
| C96 | 0.0 | μM |
| C97 | 0.1 | μM |
| C98 | 2.2 | μM |
| C99 | 2.8 | μM |
| C100 | 1.0 | μM |
| C101 | >20 | |
| C102 | 0.118 | μM |
| C103 | 0.736 | μM |
| C104 | 0.006 | μM |
| C105 | 0.0006 | μM |
| C106 | 0.0001 | μM |
| C107 | 6.792 | μM |
| C108 | 0.0012 | μM |
| C109 | 0.002 | μM |
| C110 | 0.0014 | μM |
| C111 | 0.4911 | μM |
| C112 | 0.1850 | μM |
| C113 | 0.1333 | μM |
| C114 | 0.0111 | μM |
| C115 | 0.0157 | μM |
| C116 | 4.8437 | μM |
| C117 | 0.0553 | μM |
| C118 | 0.8685 | μM |
| C119 | 0.7962 | μM |
| C120 | 1.0555 | μM |
| C121 | 0.1775 | μM |
| C122 | 0.4559 | μM |
| C123 | 0.2945 | μM |
| C124 | 0.3245 | μM |
| C125 | 0.0201 | μM |
| C126 | 1.1927 | μM |
| C127 | 0.0036 | μM |
| C128 | 0.8984 | μM |
| C129 | >20 | |
| C130 | >20 | |
| C131 | 15.81 | μM |
| C132 | 0.060 | μM |
| C133 | 0.0000855 | μM |
| C134 | 0.767 | μM |
| C135 | 1.8901 | μM |
| C136 | 0.00045 | μM |
| C137 | 0.0436 | μM |
| C138 | 0.2454 | μM |
| C139 | 3.1425 | μM |
| C140 | 0.4049 | μM |
| C141 | 0.0308 | μM |
| C142 | 0.0351 | μM |
| C143 | 0.0036 | μM |
| C144 | 2.0192 | μM |
| C145 | 4.3001 | μM |
| C146 | >20 | |
| C147 | 2.998 | μM |
| C148 | 0.9733 | μM |
| C149 | 3.965 | μM |
| C150 | >20 | |
| C151 | 3.669 | μM |
| C152 | 0.0118 | μM |
| C153 | >20 | |
| C154 | 1.480 | μM |
| C155 | 1.5618 | μM |
| C156 | 0.0528 | μM |
| C157 | 0.0001 | μM |
| C158 | 2.6117 | μM |
| C159 | 3.4899 | μM |
| C160 | 0.0247 | μM |
| C161 | 2.9937 | μM |
| C162 | 0.0922 | μM |
| C163 | 1.8127 | μM |
| C164 | 0.1440 | μM |
| C165 | 0.1057 | μM |
| C166 | 0.014 | μM |
| C167 | 2.0544 | μM |
| C168 | 5.441 | μM |
| C169 | 5.9886 | μM |
| C170 | 1.8397 | μM |
| C171 | 10.9692 | μM |
| C172 | 1.6704 | μM |
| C173 | 1.4331 | μM |
| C174 | 0.0082 | μM |
| C175 | 0.3862 | μM |
| C176 | 1.5674 | μM |
| C177 | 4.3893 | μM |
| C178 | 0.0710 | μM |
| C179 | 0.000015 | μM |
| C180 | 0.0566 | μM |
| C181 | 0.000076 | μM |
| C182 | 2.4676 | μM |
| C183 | 0.2490 | μM |
| C184 | 2.3188 | μM |
| C185 | 0.1426 | μM |
| C186 | 1.2417 | μM |
| C187 | 0.1086 | μM |
| C188 | 1.8928 | μM |
| C189 | 0.3398 | μM |
| C190 | 0.0445 | μM |
| C191 | 0.3052 | μM |
| C192 | 0.6169 | μM |
| C193 | 0.0007 | μM |
| C194 | 0.4850 | μM |
| C195 | 2.0137 | μM |
| C196 | 0.1082 | μM |
| C197 | 0.6200 | μM |
| C198 | 0.0047 | μM |
| C199 | 0.1961 | μM |
| C200 | 0.00065 | μM |
| C201 | 0.0044 | μM |
| C202 | 0.6444 | μM |
| C203 | 0.0105 | μM |
| C204 | 0.224 | μM |
| C205 | 2.559 | μM |
| C206 | 5.109 | μM |
| C207 | 0.315 | μM |
| C208 | 1.046 | μM |
| C209 | 1.875 | μM |
| C210 | 2.647 | μM |
| C211 | 0.339 | μM |
| C212 | 0.194 | μM |
| C213 | 0.257 | μM |
| C214 | 0.011 | μM |
| C215 | 20.000 | μM |
| C216 | 3.102 | μM |
| C217 | 3.991 | μM |
| C218 | 1.847 | μM |
| C219 | 20.000 | μM |
| C220 | 1.832 | μM |
| C221 | 20.000 | μM |
| C222 | 0.235 | μM |
| C223 | 0.781 | μM |
| C224 | 0.183 | μM |
| C225 | 1.01 | μM |
| C226 | 0.057 | μM |
| C227 | 0.059 | μM |
| C228 | 0.12 | μM |
| C229 | 0.0008 | μM |
| C230 | 0.07 | μM |
| C231 | 1.8 | μM |
| C232 | 15.1 | μM |
| C233 | 0.020 | μM |
| C234 | 0.0008 | μM |
| C235 | 0.071 | μM |
| C236 | 0.271 | μM |
| C237 | 0.003 | μM |
| C238 | 0.0001 | μM |
| C239 | >20 | |
| C240 | 0.23 | μM |
| C241 | 2.35 | μM |
| C242 | 0.074 | μM |
| C243 | >20 | |
| C244 | 1.73 | μM |
| C245 | 1.98 | μM |
| C246 | 0.52 | μM |
| C247 | 0.08 | μM |
| C248 | 6.93 | μM |
| C249 | >20 | |
| C250 | 0.01 | μM |
| C251 | 0.000008 | μM |
| C252 | 0.00010 | μM |
| C253 | 0.000030 | μM |
| C254 | 4.27 | μM |
| C255 | 0.07 | μM |
| C256 | 0.32 | μM |
| C257 | >20 | |
| C258 | 0.09 | μM |
| C259 | 0.05 | μM |
| C260 | 1.14 | μM |
| C261 | 16.97 | μM |
| C262 | 0.02 | μM |
| C263 | 0.02 | μM |
| C264 | 0.68 | μM |
| C265 | 0.84 | μM |
| C266 | 0.02 | μM |
| C267 | 0.10 | μM |
| C268 | 0.02 | μM |
| C269 | 0.07 | μM |
| C270 | 4.41 | μM |
| C271 | 2.00 | μM |
| C272 | 0.32 | μM |
| C273 | 5.35 | μM |
| C274 | 12.02 | μM |
| C275 | 0.02 | μM |
| C276 | 0.06 | μM |
| C277 | 0.71 | μM |
| C278 | 0.02 | μM |
| C279 | 6.59 | μM |
| C280 | >20 | |
| C281 | 2.48 | μM |
| C282 | 0.04 | μM |
| C283 | >20 | |
| C284 | 1.57 | μM |
| C285 | 0.08 | μM |
| C286 | 0.07 | μM |
| C287 | >20 | |
| C288 | 2.23 | μM |
| C289 | 0.37 | μM |
| C290 | 0.508 | μM |

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A compound having a structure of Formula (I), or a pharmaceutically acceptable salt thereof: 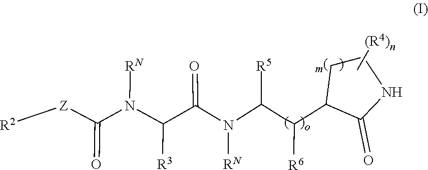
wherein
Z is O, NR¹, or a bond;
each R^{N} is independently H or C₁₋₆alkyl;
R¹ is C₅₋₈carbocyclyl optionally substituted with C₁₋₆alkylene-C₆₋₁₀aryl, or 5- to 8-membered N-heterocycle, wherein the ring nitrogen is optionally substituted with COO—C₁₋₆alkyl;
R² is C₁₋₆alkyl, C₁₋₆alkylene-C₅₋₈carbocyclyl, 4-10 membered heterocyclyl having 1-3 ring heteroatoms selected from N, O, and S, C₁₋₆alkylene-C₆₋₁₀aryl, or C₀₋₆alkylene-5-10 membered heteroaryl having 1-3 ring heteroatoms selected from N, O, and S, wherein C₁₋₆alkylene is optionally substituted with 1-3 R⁷, and the carbocyclyl, heterocyclyl, aryl, and heteroaryl is optionally substituted with 1-2 substituents independently selected from halo, C₁₋₆alkoxy, C₁₋₆alkyl, C₁₋₆haloalkyl, C₁₋₆alkylene-C₆₋₁₀aryl, O—C₁₋₆alkylene-C₆₋₁₀aryl, and CO₂C₁₋₆alkyl;
R³ is C₁₋₆alkyl, C₂₋₆alkenyl, C₂₋₆alkynyl, C₁₋₆alkylene-C₅₋₈carbocyclyl, C₀₋₆alkylene-C₆₋₁₀aryl optionally substituted with 1-2 halo, or an amino acid side chain;
each R⁴ is independently halo, OH, CN, C₁₋₆alkyl, C₁₋₆haloalkyl, C₁₋₆alkyl-OH, C₂₋₆alkenyl, C₂₋₆alkynyl, C₁₋₆alkoxy, C₃₋₆alkyloxyalkyl, oxo (═O), NR^{A}SO₂R^{B}, SO₂NR^{A}R^{B}, COOR^{A}, C₀₋₄alkylene-C₆₋₁₀aryl, C₀₋₄alkylene-(5-12 membered heteroaryl having 1-3 ring heteroatoms selected from N, O, and S), or C₀₋₄alkylene-(4-12 membered heterocycle having 1-3 ring heteroatoms selected from N, O, and S); and the aryl, heteroaryl, and heterocycle is optionally substituted with 1-2 substituents independently selected from halo, C₁₋₆alkyl, and COO—C₁₋₆alkyl, or
two R⁴ with the carbon or carbons to which they are attached combine to form a spiro or fused 3-12 membered carbocyclic or heterocylic ring having 1-3 ring heteroatoms selected from N, O, and S, which is optionally substituted with 1-2 substituents independently selected from halo, C₁₋₆alkyl, C₁₋₆alkylene-O—C₁₋₆alkyl, C(O)—C₁₋₆alkyl, SO₂—C₁₋₆alkyl, C(O)—C₁₋₆alkyl, and COO—C₁₋₆alkyl;
R⁵ is C₁₋₆alkylene-OH, C₁₋₆alkylene-OH substituted with PO(OCH₂CH₂)₂, C₁₋₆alkylene-OH substituted with SO₃H, —[C(O)]₁₋₂-(4-8 membered heterocycle having 1-3 ring heteroatoms selected from N, O, and S), —[C(O)]₁₋₂—NR^{N}R^{N}, C(O)—Y—H, or —[C(O)]₁₋₂—NR^{N}—Y—X-A, wherein A is H, C₃₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl;
Y is a bond, C₁₋₆alkylene, C₁₋₆alkylene-O—C₁₋₆alkylene, or C₁₋₆alkenylene, wherein C₁₋₆alkylene and C₁₋₆alkenylene are optionally substituted with 1-3 substituents independently selected from halo, OH, NR^{N}R^{N}, and C₁₋₆alkoxy;
X is a bond, NR^{N}R^{N}, C(O), SO₂, or OC(O);
each R⁶ is independently H, C₁₋₆alkylene-OH, C₁₋₆alkylene-OH substituted with PO(OCH₂CH₂)₂, C₁₋₆alkylene-OH substituted with SO₃H, CHO, or C(O)-(4-8 membered heterocycle having 1-3 ring heteroatoms selected from N, O, and S);
each R⁷ is independently halo, C₁₋₆haloalkyl, C₂₋₆alkenyl, C₃₋₅carbocyclyl, or C₀₋₆alkylene-C₆₋₁₀aryl, and C₆₋₁₀aryl is optionally substituted with 1-2 halo, or
two R⁷ with the carbon or carbons to which they are attached combine to form a spiro or fused C₃₋₆carbocyclyl ring;
R^{A} and R^{B} are each independently H, C₁₋₆alkyl, C₁₋₆haloalkyl, C₁₋₆alkoxy, C₂₋₆alkenyl, C₂₋₆alkynyl, C₃₋₆cycloalkyl, C₀₋₆alkylene-C₆₋₁₀ aryl, C₀₋₆ alkylene-5-8 membered heteroaryl having 1-3 ring heteroatoms selected from N, O, and S;
n is 0-3;
m is 0-5; and
o is 0-5.

2. The compound or salt according to claim 1, wherein each R^{N} is H.

3. The compound or salt according to claim 1 or 2, wherein Z is NR¹.

4. The compound or salt according to claim 3, wherein R¹ is C₅₋₈carbocyclyl optionally substituted with C₁₋₆alkylene-C₆₋₁₀aryl.

5. The compound or salt according to claim 4, wherein R¹ is unsubstituted C₅₋₈carbocyclyl.

6. The compound or salt according to claim 5, wherein R¹ is unsubstituted C₅₋₆carbocyclyl.

7. The compound or salt according to claim 3, wherein R¹ is C₅₋₈carbocyclyl substituted with C₁₋₆alkylene-C₆₋₁₀aryl.

8. The compound or salt according to claim 7, wherein R¹ is C₅₋₆carbocyclyl substituted with C₁₋₆alkylene-C₆₋₁₀aryl.

9. The compound or salt according to claim 7 or 8, wherein R¹ is C₅₋₆carbocyclyl substituted with benzyl.

10. The compound or salt according to claim 3, wherein R¹ is 5- to 8-membered N-heterocycle, wherein the ring nitrogen is substituted with COO—C₁₋₆alkyl.

11. The compound or salt according to claim 10, wherein R¹ is 6-membered N-heterocycle, wherein the ring nitrogen is substituted with COO—C₁₋₆alkyl.

12. The compound or salt according to claim 10 or 11, wherein the heterocycle ring nitrogen is substituted with COO-t-butyl.

13. The compound or salt according to claim 1 or 2, wherein Z is O.

14. The compound or salt according to any one of claims 1 to 13, wherein R² is C₁₋₆alkyl.

15. The compound or salt according to claim 12, wherein R² is methyl.

16. The compound or salt according to any one of claims 1 to 13, wherein R² is C₁₋₆alkylene-C₆₋₁₀aryl.

17. The compound or salt according to claim 16, wherein R² is benzyl.

18. The compound or salt according to any one of claims 1 to 13, wherein R² is C₁₋₆alkylene-C₅₋₈carbocyclyl.

19. The compound or salt according to any one of claims 1 to 13, wherein R² is C₁₋₆alkylene-5-10 membered heteroaryl having 1-3 ring heteroatoms selected from N, O, and S.

20. The compound or salt according to any one of claims 16 to 19, wherein R² is substituted with 1-2 substituents independently selected from halo, C₁₋₆alkoxy, C₁₋₆alkyl, C₁₋₆haloalkyl, and CO₂C₁₋₆alkyl.

21. The compound or salt according to any one of claims 1 to 20, wherein R³ is C₁₋₆alkyl.

22. The compound or salt according to claim 21, wherein R³ is

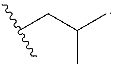 [SMILES: *CC(C)C]

23. The compound or salt according to any one of claims 1 to 20, wherein R³ is C₁₋₆alkylene-C₅₋₈carbocyclyl.

24. The compound or salt according to claim 23, wherein R³ is

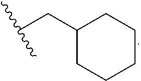 [SMILES: *CC1CCCCC1]

25. The compound or salt according to any one of claims 1 to 20, wherein R³ is C₂₋₆ alkenyl or C₂₋₆ alkynyl.

26. The compound or salt according to any one of claims 1 to 20, wherein R³ is C₀₋₆alkylene-C₆₋₁₀aryl.

27. The compound or salt according to any one of claims 1 to 20, wherein R³ is an amino acid side chain.

28. The compound or salt according to claim 27, wherein R³ is methyl, isopropyl, isobutyl, sec-butyl, CH₂CH₂SCH₃, CH₂-indolyl, benzyl, CH₂OH, CH(OH)CH₃, CH₂SH, CH₂-(4-OH-phenyl), CH₂C(O)NH₂, CH₂CH₂C(O)NH₂, CH₂COOH, CH₂CH₂COOH, CH₂CH₂CH₂CH₂NH₂, CH₂CH₂CH₂NHC(NH)NH₂, or imidazolyl.

29. The compound or salt according to any one of claims 1 to 28, wherein n is 0.

30. The compound or salt according to any one of claims 1 to 28, wherein n is 1, 2, or 3.

31. The compound or salt according to claim 30, wherein n is 1 or 2.

32. The compound or salt according to claim 30 or 31, wherein each R⁴ is independently C₁₋₆alkyl, oxo (═O), C₀₋₄alkylene-C₆₋₁₀aryl, C₀₋₄alkylene-(5-12 membered heteroaryl having 1-3 ring heteroatoms selected from N, O, and S), or C₀₋₄alkylene-(4-12 membered heterocycle having 1-3 ring heteroatoms selected from N, O, and S); and the aryl, heteroaryl, and heterocycle is optionally substituted with 1-2 substituents independently selected from halo, C₁₋₆alkyl, and COO—C₁₋₆alkyl.

33. The compound or salt according to claim 30 or 31, wherein at least one R⁴ is halo, OH, CN, C₁₋₆haloalkyl, C₁₋₆alkyl-OH, C₂₋₆alkenyl, C₂₋₆alkynyl, C₁₋₆alkoxy, C₃₋₆alkyloxyalkyl, NR^{A}SO₂R^{B}, SO₂NR^{A}R^{B}, or COOR^{A}.

34. The compound or salt according to claim 31, wherein n is 2 and two R⁴ with the carbon or carbons to which they are attached combine to form a spiro or fused 5-12 membered carbocycle or heterocylic ring having 1-3 ring heteroatoms selected from N, O, and S, which is optionally substituted with 1-2 substituents independently selected from halo, C₁₋₆alkyl, C(O)—C₁₋₆alkyl, and COO—C₁₋₆alkyl.

35. The compound or salt according to claim 34, wherein the two R⁴ with the carbon to which they are attached combine to form a spiro 5-12 membered carbocycle or heterocylic ring having 1-3 ring heteroatoms selected from N, O, and S, which is optionally substituted with 1-2 substituents independently selected from halo, C₁₋₆alkyl, C(O)—C₁₋₆alkyl, and COO—C₁₋₆alkyl.

36. The compound or salt according to claim 34, wherein the two R⁴ with the carbons to which they are attached combine to form a fused 5-12 membered carbocycle or heterocylic ring having 1-3 ring heteroatoms selected from N, O, and S, which is optionally substituted with 1-2 substituents independently selected from halo, C₁₋₆alkyl, C(O)—C₁₋₆alkyl, and COO—C₁₋₆alkyl.

37. The compound or salt according to any one of claims 1 to 36, wherein R⁵ is C₁₋₆alkylene-OH, C₁₋₆alkylene-OH substituted with PO(OCH₂CH₂)₂, C₁₋₆alkylene-OH substituted with SO₃H, C(O)-(4-8 membered heterocycle having 1-3 ring heteroatoms selected from N, O, and S), CONR^{N}R^{N}, C(O)—C(O)N(R^{N})₂, CHO, or C(O)—C(O)NR^{N}—Y—X-A, wherein A is C₃₋₈carbocyclyl, 4-12-membered heterocycle having 1-3 ring heteroatoms selected from N, O, and S, C₆₋₁₀aryl, or 5-8-membered heteroaryl having 1-3 ring heteroatoms selected from N, O, and S, and the carbocyclyl, heterocyclyl, aryl, or heteroaryl is optionally substituted with 1-2 substituents independently selected from halo, C₁₋₆alkyl, and COO—C₁₋₆alkyl.

38. The compound or salt according to any one of claims 1 to 37, wherein Y is C₁₋₆alkylene optionally substituted with 1-3 substituents independently selected from halo, OH, NR^{N}R^{N}, and C₁₋₆alkoxy.

39. The compound or salt according to any one of claims 1 to 38, wherein R² is C₁₋₆alkyl, C₀₋₄alkylene-C₅₋₈carbocyclyl, 4-10 membered heterocyclyl having 1-3 ring heteroatoms selected from N, O, and S, C₁₋₆alkylene-C₆₋₁₀aryl, 5-10 membered heteroaryl having 1-3 ring heteroatoms selected from N, O, and S, or C₁₋₆alkylene-5-10 membered heteroaryl having 1-3 ring heteroatoms selected from N, O, and S, wherein C₁₋₆alkylene is substituted with 1-3 R⁷.

40. The compound or salt according to claim 39, wherein C₁₋₆alkylene is substituted with 1 R⁷.

41. The compound or salt according to claim 39, wherein C₁₋₆alkylene is substituted with 2 R⁷.

42. The compound or salt according to claim 39, wherein C₁₋₆alkylene is substituted with 3 R⁷.

43. The compound or salt according to any one of claims 39 to 42, wherein at least one R⁷ is C₀₋₆alkylene-C₆₋₁₀aryl optionally substituted with 1-2 halo.

44. The compound or salt according to claim 43, wherein at least one R⁷ is phenyl optionally substituted with 1-2 halo.

45. The compound or salt according to claim 43 or 44, wherein at least one R⁷ is phenyl optionally substituted with 1 halo.

46. The compound or salt according to any one of claims 1 to 39, 41, and 42, wherein two R⁷ with the carbon or carbons to which they are attached combine to form a spiro or fused C₃₋₆carbocyclyl ring.

47. The compound or salt according to claim 46, wherein two R⁷ with the carbon to which they are attached combine to form a spiro C₃₋₆carbocyclyl ring.

48. The compound or salt according to claim 46, wherein two R⁷ with the carbons to which they are attached combine to form a fused C₃₋₆carbocyclyl ring.

49. The compound or salt according to any one of claims 1 to 48, wherein o is 0.

50. The compound or salt according to any one of claims 1 to 48, wherein o is 1-5.

51. The compound or salt according to claim 50, wherein o is 1 or 2.

52. The compound or salt according to claim 50 or 51, wherein each R⁶ is H.

53. The compound or salt according to claim 50 or 51, wherein at least one R⁶ is C₁₋₆alkylene-OH, C₁₋₆alkylene-OH substituted with PO(OCH₂CH₂)₂, C₁₋₆alkylene-OH substituted with SO₃H, CHO, or C(O)-(4-8 membered heterocycle having 1-3 ring heteroatoms selected from N, O, and S).

54. The compound or salt according to any one of claims 1 to 53, wherein m is 0.

55. The compound or salt according to any one of claims 1 to 53, wherein m is 1.

56. The compound or salt according to any one of claims 1 to 53, wherein m is 2.

57. The compound or salt according to any one of claims 1 to 56, wherein R⁵ is C₁₋₆alkylene-OH, C₁₋₆alkylene-OH substituted with PO(OCH₂CH₂)₂, C₁₋₆alkylene-OH substituted with SO₃H, CHO, C(O)-(4-8 membered heterocycle having 1-3 ring heteroatoms selected from N, O, and S), or CONR^{N}R^{N}.

58. The compound or salt according to any one of claims 1 to 56, wherein R⁵ is C(O)—C(O)NR^{N}—Y—X-A.

59. The compound or salt according to claim 58, wherein Y is C₁₋₆alkylene.

60. The compound or salt according to claim 58 or 59, wherein X is a bond.

61. The compound or salt according to claim 58 or 59, wherein X is NR^{N}R^{N}, C(O), SO₂, or OC(O).

62. The compound or salt according to any one of claims 58 to 61, wherein A is C₅₋₈carbocyclyl or C₆₋₁₀aryl, and optionally substituted with 1-2 substituents independently selected from halo, C₁₋₆alkyl, and COO—C₁₋₆alkyl.

63. The compound or salt according to any one of claims 58 to 61, wherein A is 4-12-membered heterocycle having 1-3 ring heteroatoms selected from N, O, and S, or 5-8-membered heteroaryl having 1-3 ring heteroatoms selected from N, O, and S, optionally substituted with 1-2 substituents independently selected from halo, C₁₋₆alkyl, and COO—C₁₋₆alkyl.

64. The compound or salt according to claim 63, wherein A comprises pyridyl.

65. A compound as recited in Table A, or a pharmaceutically acceptable salt thereof.

66. A compound as recited in Table B, or a pharmaceutically acceptable salt thereof.

67. A pharmaceutical formulation comprising the compound or salt according to any one of claims 1 to 66 and a pharmaceutically acceptable excipient.

68. A method for treating or preventing a viral infection in a host, comprising administering to the host a therapeutic amount of the compound or salt according to any one of claims 1 to 66.

69. The method according to claim 68, wherein the viral infection is coronavirus infection, calicivirus infection, or picornavirus infection.

70. The method according to claim 69, wherein the viral infection is a calicivirus infection.

71. The method according to claim 70, wherein the calicivirus infection is a norovirus infection.

72. The method according to claim 69, wherein the viral infection is a coronavirus infection.

73. The method according to claim 72, wherein the coronavirus infection is severe acute respiratory syndrome (SARS), Middle East respiratory syndrome (MERS), or Coronavirus disease 2019 (COVID-19).

74. The method according to claim 69, wherein the viral infection is a picornavirus infection.

75. The method according to claim 74, wherein the picornavirus infection is rhinovirus infection.

76. The method according to claim 75, wherein the rhinovirus infection is a rhinovirus A, rhinovirus B, or rhinovirus C infection.

77. The method according to claim 72, wherein the coronavirus is an alpha coronavirus.

78. The method according to claim 72, wherein the coronavirus is a beta coronavirus.
